(19) United States
(12) Patent Application Publication
(10) Pub. No.: US 2025/0223339 A1
(43) Pub. Date: Jul. 10, 2025

(54) SARS-COV2 ANTIBODIES AND USES THEREOF

(71) Applicant: Invivyd, Inc., Waltham, MA (US)

(72) Inventors: Laura Walker, Waltham, MA (US); Chengzi I. Kaku, Lebanon, NH (US)

(21) Appl. No.: 18/852,870

(22) PCT Filed: Apr. 1, 2023
(86) PCT No.: PCT/US2023/065250

§ 371 Date: Sep. 30, 2024

Related U.S. Application Data
(60) Provisional application No. 63/326,333, filed on Apr. 1, 2022.
(60) Provisional application No. 63/396,003, filed on Aug. 8, 2022.
(60) Provisional application No. 63/408,980, filed on Sep. 22, 2022.
(60) Provisional application No. 63/452,253, filed on Mar. 15, 2023.

(51) Int. Cl. C07K 16/10

(52) U.S. Cl. CPC C07K 16/1003; C07K 2317/33; C07K 2317/76; C07K 2317/92

(57) ABSTRACT

The present disclosure is directed to antibodies and antigen binding fragments thereof having binding specificity for the S protein of coronaviruses (CoV-S), such as the S protein of the SARS coronavirus 2 (SARS-COV-2-S), including neutralizing antibodies. The antibodies and antigen binding fragments thereof comprise the sequences of the VH, VL, and CDR polypeptides described herein, and the polynucleotides encoding them. The disclosure contemplates conjugates of anti-CoV-S antibodies and binding fragments thereof conjugated to one or more functional or detectable moieties. Methods of making said anti-CoV-S antibodies and antigen binding fragments thereof are also contemplated. Other embodiments of the disclosure contemplate using anti-CoV-S antibodies, and binding fragments thereof, for the diagnosis, assessment, and treatment of diseases and disorders associated with coronaviruses or the S protein thereof and conditions where neutralization or inhibition of coronaviruses or the S protein thereof would be therapeutically beneficial.

### RELATED APPLICATIONS

This application claims the benefit of priority to U.S. Provisional Application 63/326,333, filed Apr. 1, 2022; U.S. Provisional Application No. 63/396,003, filed Aug. 8, 2022; U.S. Provisional Application No. 63/408,980, filed Sep. 22, 2022; and U.S. Provisional Application No. 63/452,253, filed Mar. 15, 2023. The entire contents of each of these applications are incorporated herein by reference.

### SEQUENCE LISTING

The instant application contains a Sequence Listing which has been submitted electronically in XML file format and is hereby incorporated by reference in its entirety. Said XML copy, created on Mar. 27, 2023, is named 132280-00720_SL.xml and is 7,171,527 bytes in size.

### FIELD

This disclosure generally pertains to antibodies and antigen-binding fragments thereof, preferably human antibodies and antigen-binding fragments and/or affinity-matured variants thereof, recombinant cells engineered to express such antibodies, and compositions containing such antibodies and antigen-binding fragments thereof, wherein such antibodies and antigen-binding fragments thereof bind to the S protein of coronaviruses (“CoV-S”) and therapeutic and diagnostic uses for the antibodies, antigen-binding fragments, and compositions thereof.

### BACKGROUND

Coronaviruses (“CoV”) are genetically classified into four major genera: the Alphacoronavirus genus (ACoV genus); the Betacoronavirus genus (BCoV genus); the Gammacoronavirus genus (CCoV genus); and Deltacoronavirus genus (DCoV genus), and while ACoV and BCoV primarily infect mammals CCOV and DCoV predominantly infect birds (Wu A. et al., Cell Host Microbe. 2020 Mar. 11; 27 (3): 325-328). Coronaviruses that infect humans were first identified in the mid-1960s, and currently, seven confirmed CoV species are known as human pathogens. Four CoV species, the HCoV-HKU1 and HCoV-OC43 from the BCoV genus and the HCoV-229E and HCoV-NL63 from the ACoV genus, are endemic species in humans and cause mild respiratory symptoms, mostly in pediatric patients (Brielle E. S., et al., BioRxiv reprint, 2020.03.10). The other three human CoV species, the SARS-COV, the MERS-COV, and the SARS-COV-2 (also known as “2019-nCOV”), all of which are from the BCoV genus, have caused severe outbreaks, including the Severe Acute Respiratory Syndrome (SARS) outbreak in 2002-2003, the Middle East Respiratory Syndrome (MERS) outbreak in 2012-2013, and the current (2019-) pandemic of the coronavirus disease of 2019 (“COVID-19”).

The genome of coronaviruses, whose size ranges between approximately 26,000 and 32,000 bases, includes a variable number (from 6 to 11) of open reading frames (“ORFs”) (Wu A. et al., Cell Host Microbe. 2020; 27 (3): 325-328). The first ORF encodes 16 non-structural proteins (“nsps”), and the remaining ORFs encode accessory proteins and structural proteins. The four major structural proteins are the spike surface glycoprotein (“'S protein” or “S” or “spike protein”), small envelope protein (“E protein” or “E”), matrix protein (“M protein” or “M”), and nucleocapsid protein (“N protein”, or “N”).

The S protein, which plays an essential role in binding to receptors on the host cell and determines host tropism (Zhu Z. et al., Infect Genet Evol. 2018; 61:183-184), forms homotrimers protruding from the viral surface (Li F. Annu Rev Virol. 2016 Sep. 29; 3 (1): 237-261. Epub 2016 Aug. 25). The S protein is processed into two non-covalently associated subunits, S1 and S2, and each monomer in the trimeric S assembly is a heterodimer of S1 and S2 subunits. Cryo-EM studies have revealed that the S1 subunit is comprised of four domains: an N-terminal domain (NTD), a C-terminal domain (CTD), and two subdomains (Walls A. C. et al., Nature 531, 114-117 (2016).; Tortorici M. A. and Veesler D., Adv Virus Res. 2019; 105:93-116. doi: 10.1016/bs.aivir.2019.08.002. Epub 2019 Aug. 22.; Wrapp D. et al., Science 367, 1260-1263 (2020)). The CTD functions as the receptor-binding domain (RBD) for both SARS-COV and SARS-COV-2 (Li F. J Virol. 2015 February; 89 (4): 1954-64. doi: 10.1128/JVI.02615-14. Epub 2014 Nov. 26). The S2 subunit contains the fusion peptide, heptad repeat 1 and 2, and a transmembrane domain, all of which are required to mediate fusion of the viral and host cell membranes.

SARS-COV and SARS-COV-2 bind to and use angiotensin-converting enzyme 2 (ACE2) of a host cell as a receptor to enter the host cells (Ge X. Y. et al., Nature. 2013 Nov. 28; 503 (7477): 535-8; Hoffmann M. et al., Cell. 2020 Mar. 4). The motif within the RBD that particularly binds to RCE2 is often referred to as the “ACE2-binding motif”. SARS-COV can also use CD209L (also known as L-SIGN) as an alternative receptor (Jeffers S. A. et al., Proc Natl Acad Sci USA. 2004 Nov. 2; 101 (44): 15748-53. Epub 2004 Oct. 20). In contrast, MERS-COV binds dipeptidyl peptidase 4 (“DPP4”, also known as CD26) of the host cell via a different RBD of the S protein.

Cell entry of coronaviruses often depends also on priming of the S protein by host cell proteases. Recently, SARS-COV-2 was found to use the serine protease TMPRSS2 for S protein priming and ACE2 for entry (Wu A. et al., Cell Host Microbe. 2020; 27 (3): 325-328; Hoffmann M. et al., Cell. 2020 Mar. 4).

The genome of SARS-COV-2 is about 29.8 kb nucleotides and encodes 15 non-structural proteins, four structural proteins (S, E, M, and N) and eight accessory proteins (3a, 3b, p6, 7a, 7b, 8b, 9b, and orf14) (Wu A. et al., Cell Host Microbe. 2020 Mar. 11; 27 (3): 325-328). While SARS-COV-2 is genetically close to a SARS-like bat CoV and also to SARS-COV, a number of sequence differences have been identified. When SARS-COV-2 is compared to SARS-COV or SARS-like bat CoV, 380 amino acid differences or substitutions were found, 27 of which are in the S protein, including 6 substitutions in the RBD at amino acid region 357-528 (but not in the receptor-binding motifs that directly interact with ACE2) and 6 substitutions in the underpinning subdomain (SD) at amino acid region 569-655.

Multiple variants of SARS-COV-2 have developed since the onset of COVID. Among those, the Omicron variant is a highly divergent variant with a high number of mutations, including 26-32 mutations in the spike protein, some of which are associated with humoral immune escape potential and higher transmissibility. Omicron is a much more rapidly spreading variant that has grown from first report on Nov. 25, 2021 to a record global spike of cases that is already several times the level of Delta. Moreover, Omicron is causing an increase in breakthrough infections among fully vaccinated individuals. As a result, the protection provided by both vaccination and prior infection is much reduced.

Accordingly, there is an unmed need for development of new treatment, e.g., antibodies, for treating SAR-COV-2, in particular, the Omicron variant.

### SUMMARY

The present invention is based on the discovery of antibodies with broad activity against all SARS-COV-2 variants of concern (VOCs) described to date. In particular, the disclosed antibodies were isolated from Omicron/BA.1 breakthrough infection donors and were shown to display broad activity against all SARS-COV-2 VOCs, including the Omicron variant BA.1 and its sublineages, e.g., BF.7, BQ.1.1, BA.2.75, and/or XBB.1. These antibodies represent promising candidates for therapeutic development and provide a framework for the development of vaccines that induce broadly neutralizing antibody responses.

Accordingly, in one aspect, the present disclosure provides an isolated antibody, or antigen-binding fragment thereof, that binds to the spike protein of a corona virus (CoV-S), wherein said antibody, or antigen-binding fragment thereof, comprises a heavy chain variable region (VH) and a light chain variable region (VL), wherein the VH comprises an amino acid sequence having at least 90%, 91%, 92%, 93%, 94%, 95%, 96%, 97%, 98%, or 99% sequence identity to, comprises, or consists of an amino acid sequence selected from the group consisting of any one of the VH sequences in Tables 3 and 5, and wherein the VL comprises an amino acid sequence having at least 90%, 91%, 92%, 93%, 94%, 95%, 96%, 97%, 98%, or 99% sequence identity to, comprises, or consists of an amino acid sequence selected from the group consisting of any one of the VL sequences in Tables 4 and 6. In one embodiment, the VH and VL are from the same antibody in Tables 3-6. In another embodiment, the VH and VL are from a different antibody in Tables 3-6.

In some embodiments, the VH comprises a VH CDR1 amino acid sequence selected from the group consisting of any VH CDR1 sequence in Tables 3 and 5, a VH CDR2 amino acid sequence selected from the group consisting of any VH CDR2 sequence in Tables 3 and 5, and a VH CDR3 amino acid sequence selected from the group consisting of any VH CDR3 sequence in Tables 3 and 5, and the VL comprises a VL CDR1 amino acid sequence selected from the group consisting of any VL CDR1 sequence in Tables 4 and 6, a VL CDR2 amino acid sequence selected from the group consisting of any VL CDR2 sequence in Tables 4 and 6, and a VL CDR3 amino acid sequence selected from the group consisting of any VL CDR3 sequence in Tables 4 and 6. In one embodiment, the CDRs are from the same antibody in Tables 3-6. In another embodiment, the CDRs are from a different antibody in Tables 3-6.

In another aspect, the present disclosure provides an isolated antibody, or antigen-binding fragment thereof, that binds to the spike protein of a corona virus (CoV-S), wherein said antibody, or antigen-binding fragment thereof, comprises a heavy chain variable region (VH) and a light chain variable region (VL), wherein the VH comprises a VH CDR1 amino acid sequence selected from the group consisting of any VH CDR1 sequence in Tables 3 and 5, a VH CDR2 amino acid sequence selected from the group consisting of any VH CDR2 sequence in Tables 3 and 5, and a VH CDR3 amino acid sequence selected from the group consisting of any VH CDR3 sequence in Tables 3 and 5, and wherein the VL comprises a VL CDR1 amino acid sequence selected from the group consisting of any VL CDR1 sequence in Tables 4 and 6, a VL CDR2 amino acid sequence selected from the group consisting of any VL CDR2 sequence in Tables 4 and 6, and a VL CDR3 amino acid sequence selected from the group consisting of any VL CDR3 sequence in Tables 4 and 6.

In some embodiments, the VH comprises an amino acid sequence having at least 90%, 91%, 92%, 93%, 94%, 95%, 96%, 97%, 98%, or 99% sequence identity to, comprises, or consists of an amino acid sequence selected from the group consisting of any one of the VH sequences in Tables 3 and 5, and the VL comprises an amino acid sequence having at least 90%, 91%, 92%, 93%, 94%, 95%, 96%, 97%, 98%, or 99% sequence identity to, comprises, or consists of an amino acid sequence selected from the group consisting of any one of the VL sequences in Tables 4 and 6.

In some embodiments, the VH comprises the VH amino acid sequence of ADI-75738, and the VL comprises the VL amino acid sequence of ADI-75738. In some embodiments, the VH comprises the VH amino acid sequence of ADI-75700, and the VL comprises the VL amino acid sequence of ADI-75700. In some embodiments, the VH comprises the VH amino acid sequence of ADI-75859, and the VL comprises the VL amino acid sequence of ADI-75859. In some embodiments, the VH comprises the VH amino acid sequence of ADI-75684, and the VL comprises the VL amino acid sequence of ADI-75684. In some embodiments, the VH comprises the VH amino acid sequence of ADI-75754, and the VL comprises the VL amino acid sequence of ADI-75754. In some embodiments, the VH comprises the VH amino acid sequence of ADI-75648, and the VL comprises the VL amino acid sequence of ADI-75648. In some embodiments, the VH comprises the VH amino acid sequence of ADI-75632, and the VL comprises the VL amino acid sequence of ADI-75632. In some embodiments, the VH comprises the VH amino acid sequence of ADI-75741, and the VL comprises the VL amino acid sequence of ADI-75741. In some embodiments, the VH comprises the VH amino acid sequence of ADI-75725, and the VL comprises the VL amino acid sequence of ADI-75725. In some embodiments, the VH comprises the VH amino acid sequence of ADI-75717, and the VL comprises the VL amino acid sequence of ADI-75717. In some embodiments, the VH comprises the VH amino acid sequence of ADI-75706, and the VL comprises the VL amino acid sequence of ADI-75706. In some embodiments, the VH comprises the VH amino acid sequence of ADI-75699, and the VL comprises the VL amino acid sequence of ADI-75699. In some embodiments, the VH comprises the VH amino acid sequence of ADI-75747, and the VL comprises the VL amino acid sequence of ADI-75747. In some embodiments, the VH comprises the VH amino acid sequence of ADI-75773, and the VL comprises the VL amino acid sequence of ADI-75773. In some embodiments, the VH comprises the VH amino acid sequence of ADI-75696, and the VL comprises the VL amino acid sequence of ADI-75696. In some embodiments, the VH comprises the VH amino acid sequence of VYD223 (also known as ADI-75865), and the VL comprises the VL amino acid sequence of VYD223. In some embodiments, the VH comprises the VH amino acid sequence of VYD224 (also known as ADI-80707), and the VL comprises the VL amino acid sequence of VYD224. In some embodiments, the VH comprises the VH amino acid sequence of ADI-75864, and the VL comprises the VL amino acid sequence of ADI-75864. In some embodiments, the VH comprises the VH amino acid sequence of ADI-75620, and the VL comprises the VL amino acid sequence of ADI-75620. In some embodiments, the VH comprises the VH amino acid sequence of VYD225, and the VL comprises the VL amino acid sequence of VYD225.

In some embodiments, the antibody or antibody-binding fragment thereof comprises the VH CDR1, VH CDR2, VH CDR3, VL CDR1, VL CDR2, and VL CDR3 amino acid sequences of ADI-75738. In some embodiments, the antibody or antibody-binding fragment thereof comprises the VH CDR1, VH CDR2, VH CDR3, VL CDR1, VL CDR2, and VL CDR3 amino acid sequences of ADI-75700. In some embodiments, the antibody or antibody-binding fragment thereof comprises the VH CDR1, VH CDR2, VH CDR3, VL CDR1, VL CDR2, and VL CDR3 amino acid sequences of ADI-75859. In some embodiments, the antibody or antibody-binding fragment thereof comprises the VH CDR1, VH CDR2, VH CDR3, VL CDR1, VL CDR2, and VL CDR3 amino acid sequences of ADI-75684. In some embodiments, the antibody or antibody-binding fragment thereof comprises the VH CDR1, VH CDR2, VH CDR3, VL CDR1, VL CDR2, and VL CDR3 amino acid sequences of ADI-75754. In some embodiments, the antibody or antibody-binding fragment thereof comprises the VH CDR1, VH CDR2, VH CDR3, VL CDR1, VL CDR2, and VL CDR3 amino acid sequences of ADI-75648. In some embodiments, the antibody or antibody-binding fragment thereof comprises the VH CDR1, VH CDR2, VH CDR3, VL CDR1, VL CDR2, and VL CDR3 amino acid sequences of ADI-75632. In some embodiments, the antibody or antibody-binding fragment thereof comprises the VH CDR1, VH CDR2, VH CDR3, VL CDR1, VL CDR2, and VL CDR3 amino acid sequences of ADI-75741. In some embodiments, the antibody or antibody-binding fragment thereof comprises the VH CDR1, VH CDR2, VH CDR3, VL CDR1, VL CDR2, and VL CDR3 amino acid sequences of ADI-75725. In some embodiments, the antibody or antibody-binding fragment thereof comprises the VH CDR1, VH CDR2, VH CDR3, VL CDR1, VL CDR2, and VL CDR3 amino acid sequences of ADI-75717. In some embodiments, the antibody or antibody-binding fragment thereof comprises the VH CDR1, VH CDR2, VH CDR3, VL CDR1, VL CDR2, and VL CDR3 amino acid sequences of ADI-75706. In some embodiments, the antibody or antibody-binding fragment thereof comprises the VH CDR1, VH CDR2, VH CDR3, VL CDR1, VL CDR2, and VL CDR3 amino acid sequences of ADI-75699. In some embodiments, the antibody or antibody-binding fragment thereof comprises the VH CDR1, VH CDR2, VH CDR3, VL CDR1, VL CDR2, and VL CDR3 amino acid sequences of ADI-75747. In some embodiments, the antibody or antibody-binding fragment thereof comprises the VH CDR1, VH CDR2, VH CDR3, VL CDR1, VL CDR2, and VL CDR3 amino acid sequences of ADI-75773. In some embodiments, the antibody or antibody-binding fragment thereof comprises the VH CDR1, VH CDR2, VH CDR3, VL CDR1, VL CDR2, and VL CDR3 amino acid sequences of ADI-75696. In some embodiments, the antibody or antibody-binding fragment thereof comprises the VH CDR1, VH CDR2, VH CDR3, VL CDR1, VL CDR2, and VL CDR3 amino acid sequences of ADI-75864. In some embodiments, the antibody or antibody-binding fragment thereof comprises the VH CDR1, VH CDR2, VH CDR3, VL CDR1, VL CDR2, and VL CDR3 amino acid sequences of ADI-75620. In some embodiments, the antibody or antibody-binding fragment thereof comprises the VH CDR1, VH CDR2, VH CDR3, VL CDR1, VL CDR2, and VL CDR3 amino acid sequences of VYD223. In some embodiments, the antibody or antibody-binding fragment thereof comprises the VH CDR1, VH CDR2, VH CDR3, VL CDR1, VL CDR2, and VL CDR3 amino acid sequences of VYD224. In some embodiments, the antibody or antibody-binding fragment thereof comprises the VH CDR1, VH CDR2, VH CDR3, VL CDR1, VL CDR2, and VL CDR3 amino acid sequences of VYD225.

In some embodiments, SARS-COV-S comprises a sequence having at least 95% identity to the amino acid sequence of SEQ ID NO:1, and wherein SARS-COV-2-S comprises a sequence having at least 95% identity to the amino acid sequence of SEQ ID NO:5.

In some embodiments, the isolated antibody, or antigen-binding fragment thereof, cross-reacts with SARS-COV-S and SARS-COV-2-S.

In certain embodiments, an antibody or antigen-binding fragment thereof is capable of binding to a SARS-COV-2 variant. In some embodiments, the SARS-COV-2-S is a B.1.1.7 variant (alpha), a B. 1.351 variant (beta), a B.1.1.28 variant, a B. 1.429 variant, a P.1 variant, a B.1.617 variant (e.g., B.1.617.1 and B.1.617.2 (delta)), a C.37 variant, a 1.621 variant, a AY.1 variant, a 1.623 variant, a C.36 variant, a A.27 variant, a AV.1 variant, a B.1.1.482 variant, a B.1.1.523 variant, a B.1.427 variant, a AY.4 variant, a AY.11 variant, a D614G variant, or a B.1.1.529/BA.1 variant (also known as the Omicron variant) or its sub-lineages (e.g., BA1.1, BA.2, BA.2.75, BA.4, BA.5, BA.4.6, BQ.1, BQ.1.1, XBB, XBB.1, XBB.1.5, BJ.1, BM.1.1.1, BA.2.3.20, BF.7, XBC, BN.1, or CH.1.1).

In some embodiments, the antibody, or antigen-binding fragment thereof, cross-reacts with SARS-COV-S and SARS-COV-2-S.

In some embodiments, the antibody, or antigen-binding fragment thereof, binds to the receptor binding domain (RBD) or the N-terminal domain (NTD) of SARS-COV-S and/or of SARS-CoV-2-S.

In some embodiments, the antibody, or antigen-binding fragment thereof, binds to the receptor binding domain (RBD) of CoV-S from the B.1.1.529/BA.1 variant, the BA.1.1 variant, the BA.2.75 variant, the BQ.1.1 variant, and/or the XBB variant.

In some embodiments, the antibody, or antigen-binding fragment thereof, binds to the S1 subunit and/or the S2 subunit of SARS-COV-S and/or of SARS-COV-2-S.

In some embodiments, the antibody, or antigen-binding fragment thereof, binds to the ACE2-binding motif of SARS-COV-S and/or of SARS-COV-2-S.

In one embodiment, the antibody, or antigen-binding fragment thereof, competes with ACE2.

In some embodiments, the antibody, or antigen-binding fragment thereof, (a) binds to the S protein of SARS-COV and/or of SARS-COV-2; and (b) does not bind to any of the S proteins of HCoV-229E, HCoV-HKU1, HCoV-NL63, and HCoV-OC43.

In some embodiments, the antibody, or antigen-binding fragment thereof, (a) binds to the S protein of SARS-COV and/or of SARS-COV-2; and (b) binds to the S protein of at least one of HCoV-229E, HCoV-HKU1, HCoV-NL63, and HCoV-OC43.

In one embodiment, the antibody, or antigen-binding fragment thereof, binds to CoV-S with a KD value of: (i) about 100 nM or lower; (ii) about 10 nM or lower; (iii) about 1 nM or lower; (iv) about 100 pM or lower; (v) about 10 pM or lower; (vi) about 1 pM or lower; or (vii) about 0.1 pM or lower.

In some embodiments, the antibody, or antigen-binding fragment thereof, binds to the receptor binding domain (RBD) of CoV-S from the B.1.1.529/BA.1 variant, the BA.1.1 variant, the BA.2.75 variant, the BQ.1.1 variant, the XBB variant(s), the B.1351 variant, or the B.1.617.2 variant with a KD value of about 100 nM or lower, or about 10 nM or lower, or about 1 nM or lower.

In some embodiments, the dissociation constant (KD) is measured using an assay selected from the group consisting of surface plasmon resonance, ELISAs, radioimmunoassays, Western blotting, and bio-layer interferometry (BLI).

In some embodiments, the antibody, or antigen-binding fragment thereof, neutralizes SARS-CoV and/or SARS-COV-2. In some embodiments, the neutralization activity is measured using a VSV-based pseudovirus system. In some embodiments, the neutralization activity is measured using a murine leukemia virus (MLV)-based pseudovirus system. In some embodiments, the neutralization activity is measured using a PhenoSense SARS-COV-2 pseudovirus neutralizing assay.

In some embodiments, the antibody, or antigen-binding fragment thereof, neutralizes SARS-CoV and/or SARS-COV-2 at: (i) an IC50 of about 100 nM or lower, of about 50 nM or lower, of about 20 nM or lower, of about 10 nM or lower, of about 5 nM or lower, of about 2 nM or lower, of about 1 nM or lower, of about 500 pM or lower, of about 200 pM or lower, of about 100 pM or lower, of about 50 pM or lower, of about 20 pM or lower, of about 10 pM or lower, of about 5 pM or lower, of about 2 pM or lower, or of about 1 pM or lower; and/or (ii) an IC50 of about 1 μg/mL or lower, of about 500 ng/ml or lower, of about 200 ng/ml or lower, of about 100 ng/ml or lower, of about 50 ng/ml or lower, of about 20 ng/mL or lower, of about 10 ng/ml or lower, of about 20 ng/ml or lower, of about 10 mg/mL or lower, of about 5 ng/ml or lower, of about 2 ng/ml or lower, or of about 1 ng/ml or lower, in vitro. In some embodiments, the antibody, or antigen-binding fragment thereof, cross-neutralize VSV-SARS-COV-1 and VSV-SARS-COV-2 with a neutralization IC50 less than about 1 μg/mL. In some embodiments, the neutralization activity is measured using a VSV-based pseudovirus system. In some embodiments, the neutralization activity is measured using a murine leukemia virus (MLV)-based pseudovirus system. In some embodiments, the neutralization activity is measured using a PhenoSense SARS-COV-2 pseudovirus neutralizing assay.

In some embodiments, the antibody, or antigen-binding fragment thereof, neutralizes the B.1.1.529/BA.1 variant of SARS-COV-2 with an IC50 of about 100 ng/mL or lower, of about 50 ng/ml or lower, of about 40 ng/ml or lower, of about 30 ng/ml or lower, of about 20 ng/mL or lower, of about 10 mg/mL or lower, of about 5 ng/ml or lower, of about 2 ng/ml or lower, or of about 1 ng/ml or lower, in vitro. In some embodiments, the antibody, or antigen-binding fragment thereof, neutralizes the B.1.1.529/BA.1 variant of SARS-COV-2 with an IC50 of about 60 ng/ml or lower.

In some embodiments, the antibody, or antigen-binding fragment thereof, neutralizes the BA.2.75 variant, the BF.7 variant, the BQ.1.1 variant, the XBB.1 variant and/or the XBB.1.5 variant of SARS-COV-2 with an IC50 of about 200 ng/ml or lower, of about 100 ng/mL or lower, of about 50 ng/ml or lower, of about 40 ng/ml or lower, of about 30 ng/mL or lower, of about 20 ng/ml or lower, of about 10 mg/mL or lower, of about 5 ng/ml or lower, of about 2 ng/ml or lower, or of about 1 ng/ml or lower, in vitro.

In some embodiments, the antibody, or antigen-binding fragment thereof, neutralizes the BA.2.75 variant of SARS-COV-2 with an IC50 of about 40 ng/ml or lower.

In some embodiments, the antibody, or antigen-binding fragment thereof, neutralizes the BF.7 variant of SARS-COV-2 with an IC50 of about 30 ng/ml or lower.

In some embodiments, the antibody, or antigen-binding fragment thereof, neutralizes the BQ.1.1 variant of SARS-COV-2 with an IC50 of about 50 ng/mL or lower.

In some embodiments, the antibody, or antigen-binding fragment thereof, neutralizes the XBB.1 variant of SARS-COV-2 with an IC50 of about 200 ng/ml or lower.

In some embodiments, the antibody, or antigen-binding fragment thereof, neutralizes the D614G variant of SARS-COV-2 with an IC50 of about 100 ng/ml or lower, of about 50 ng/ml or lower, of about 40 ng/mL or lower, of about 30 ng/mL or lower, of about 20 ng/ml or lower, of about 10 mg/mL or lower, of about 5 ng/mL or lower, of about 2 ng/ml or lower, or of about 1 ng/ml or lower, in vitro. In some embodiments, the antibody, or antigen-binding fragment thereof, neutralizes the D614G variant of SARS-COV-2 with an IC50 of about 20 ng/ml or lower,

In some embodiments, the antibody, or antigen-binding fragment thereof, is a human, humanized, primatized or chimeric antibody, or antigen-binding fragment thereof.

In some embodiments, the antibody, or antigen-binding fragment thereof, is bispecific or multispecific. In some embodiments, the bispecific or multispecific antibody, or antigen-binding fragment thereof, comprises at least one first antigen-binding domain (“ABD”) and at least one second ABD, wherein: (i) said first ABD comprises the VH CDR1, the VH CDR2, the VH CDR3, the VL CDR1, the VL CDR2, and the VL CDR3 of a first antibody selected from Tables 3-6; and (ii) said second ABD comprises the VH CDR1, the VH CDR2, the VH CDR3, the VL CDR1, the VL CDR2, and the VL CDR3 of a second antibody selected from Tables 3-6, wherein the first anti-CoV-S antibody is same as the second anti-CoV-S antibody, or wherein the first anti-CoV-S antibody is different from the second anti-CoV-S antibody.

In some embodiments, the first anti-CoV-S antibody binds to a first CoV-S, and the second anti-CoV-S antibody binds to a second CoV-S.

In some embodiments, the first anti-CoV-S antibody and the second anti-CoV-S antibody bind to: (i) the same coronavirus species, optionally wherein the first CoV-S and the second CoV-S are (a) both of SARS-COV or (b) both of SARS-COV-2, and optionally wherein the first anti-CoV-S antibody and the second anti-CoV-S antibody bind to the same or different epitopes on a CoV-S expressed by said SARS-COV or SARS-COV-2; or (ii) different coronavirus species, optionally wherein the first CoV-S and the second CoV-S are of (a) of SARS-COV and of SARS-COV-2, respectively, or (b) of SARS-COV-2 and of SARS-COV, respectively.

In some embodiments, the bispecific or multispecific antibody, or antigen-binding fragment thereof, comprises at least one first antigen-binding domain (“ABD”) and at least one second ABD, wherein: wherein: (a) said first ABD comprises the VH CDR1, the VH CDR2, the VH CDR3, the VL CDR1, the VL CDR2, and the VL CDR3 of a first anti-CoV-S antibody selected from Tables 3-6; and (b) said second ABD binds to an antigen which is not a CoV-S, optionally wherein said antigen is a cytokine, a cytokine receptor, or an immunomodulatory polypeptide.

In some embodiments, the antibody, or antigen-binding fragment thereof, comprises a Fab, Fab2, or scFv. In some embodiments, the antibody, or antigen-binding fragment thereof, comprises a constant region, an Fc region, or at least one domain thereof.

In some embodiments, the constant region or Fc region comprises a mutation which impairs at least one effector function, optionally FcR binding, complement binding, glycosylation, complement-dependent cytotoxicity (“CDC”), or antibody-dependent cellular cytotoxicity (“ADCC”).

In some embodiments, the constant or Fc region is human. In some embodiments, the human constant or Fc region is selected from a human IgG1, IgG2, IgG3 or IgG4 constant or Fc region.

In one embodiment, the antibody, or antigen-binding fragment thereof, is a neutralizing antibody. In one embodiment, the antibody, or antigen-binding fragment thereof, is an affinity optimized antibody, or antigen-binding fragment thereof.

In one aspect, the present disclosure provides an isolated antibody, or antigen-binding fragment thereof, which competes for binding with the isolated antibody, or antigen-binding fragment thereof, as described herein.

In some embodiments, binding competition is measured using an assay selected from the group consisting of surface plasmon resonance, ELISAs, radioimmunoassays, Western blotting, and bio-layer interferometry (BLI).

In one aspect, the present disclosure provides an isolated antibody, or antigen-binding fragment thereof, which binds the same epitope as the isolated antibody, or antigen-binding fragment thereof, as described herein.

In some embodiments, epitope mapping is determined using an assay selected from the group consisting of surface plasmon resonance, ELISAs, radioimmunoassays, Western blotting, and bio-layer interferometry (BLI).

In another aspect, the present disclosure provides an affinity matured variant of any one of the isolated antibodies, or antigen-binding fragments thereof, as described herein.

In one aspect, the present disclosure provides a nucleic acid molecule, e.g., a DNA or an mRNA molecule, encoding an antibody or antigen binding fragment thereof, as disclosed herein.

In one aspect, the present disclosure provides a chimeric antigen receptor (“CAR”) comprising at least one antibody, or antigen-binding fragment thereof, as described herein.

In one aspect, the present disclosure provides an antibody drug conjugate (“ADC”) comprising: (a) at least one antibody, or antigen-binding fragment thereof, as described herein; and (b) a drug.

In some embodiments, the drug is: (i) an antiviral drug, optionally, remdesivir, favipiravir, darunavir, nelfinavir, saquinavir, lopinavir or ritonavir; (ii) an antihelminth drug, optionally ivermectin; (iii) an antiparasite drug, optionally hydroxychloroquine, chloroquine, or atovaquone; (iv) antibacterial vaccine, optionally the tuberculosis vaccine BCG; or (v) an anti-inflammatory drug, optionally a steroid such as ciclesonide, a TNF inhibitor (e.g., adalimumab), a TNF receptor inhibitor (e.g., etanercept), an IL-6 inhibitor (e.g., clazakizumab), an IL-6 receptor inhibitor (e.g., toclizumab), or metamizole; (vi) an antihistamine drug, optionally bepotastine; (vii) an ACE inhibitor, optionally moexipril; (viii) a drug that inhibits priming of CoV-S, optionally a serine protease inhibitor, further optionally nafamostat; or (ix) a cytotoxic drug, optionally daunorubicin, mitoxantrone, doxorubicin, cucurbitacin, chaetocin, chaetoglobosin, chlamydocin, calicheamicin, nemorubicin, cryptophyscin, mensacarcin, ansamitocin, mitomycin C, geldanamycin, mechercharmycin, rebeccamycin, safracin, okilactomycin, oligomycin, actinomycin, sandramycin, hypothemycin, polyketomycin, hydroxyellipticine, thiocolchicine, methotrexate, triptolide, taltobulin, lactacystin, dolastatin, auristatin, monomethyl auristatin E (MMAE), monomethyl auristatin F (MMAF), telomestatin, tubastatin A, combretastatin, maytansinoid, MMAD, MMAF, DM1, DM4, DTT, 16-GMB-APA-GA, 17-DMAP-GA, JW 55, pyrrolobenzodiazepine, SN-38, Ro 5-3335, puwainaphycin, duocarmycin, bafilomycin, taxoid, tubulysin, ferulenol, lusiol A, fumagillin, hygrolidin, glucopiericidin, amanitin, ansatrienin, cinerubin, phallacidin, phalloidin, phytosphongosine, piericidin, poronetin, phodophyllotoxin, gramicidin A, sanguinarine, sinefungin, herboxidiene, microcolin B, microcystin, muscotoxin A, tolytoxin, tripolin A, myoseverin, mytoxin B, nocuolin A, psuedolaric acid B, pseurotin A, cyclopamine, curvulin, colchicine, aphidicolin, englerin, cordycepin, apoptolidin, epothilone A, limaquinone, isatropolone, isofistularin, quinaldopeptin, ixabepilone, aeroplysinin, arruginosin, agrochelin, or epothilone.

In one aspect, the present disclosure provides a composition comprising at least one antibody, or antigen-binding fragment thereof, a CAR or an ADC as described herein. In one embodiment, the composition further comprises an additional anti-SARS-COV-S antibody, or antigen-binding fragment thereof.

In one aspect, the present disclosure provides a pharmaceutical composition comprising at least one antibody, or antigen-binding fragment thereof, a CAR or an ADC as described herein; and a pharmaceutically acceptable carrier or excipient. In one embodiment, the pharmaceutical composition further comprises an additional anti-SARS-COV-S antibody, or antigen-binding fragment thereof.

In one aspect, the present disclosure provides a method of treating infection by SARS-COV, SARS-COV-2, and/or another coronavirus optionally selected from the group consisting of MERS-CoV, HCoV-HKU1, HCoV-OC43, HCoV-229E, and HCoV-NL63, or treating a condition, symptom, disease, or disorder associated with said infection in a subject in need thereof, comprising administering to the subject a therapeutically effective amount of an antibody, or antigen-binding fragment thereof, a CAR or an ADC as described herein.

In some embodiments, the condition, symptom, disease, or disorder comprises at least one of bronchitis, pneumonia, respiratory failure, acute respiratory failure, organ failure, multi-organ system failure, pediatric inflammatory multisystem syndrome, acute respiratory distress syndrome, blood clot, a cardiac condition, myocardial injury, myocarditis, heart failure, cardiac arrest, acute myocardial infarction, dysrhythmia, venous thromboembolism, post-intensive care syndrome, shock, anaphylactic shock, cytokine release syndrome, septic shock, disseminated intravascular coagulation, ischemic stroke, intracerebral hemorrhage, microangiopathic thrombosis, psychosis, seizure, nonconvulsive status epilepticus, traumatic brain injury, stroke, anoxic brain injury, encephalitis, posterior reversible leukoencephalopathy, necrotizing encephalopathy, post-infectious encephalitis, autoimmune mediated encephalitis, acute disseminated encephalomyelitis, acute kidney injury, acute liver injury, pancreatic injury, immune thrombocytopenia, subacute thyroiditis, a gastrointestinal complication, aspergillosis, increased susceptibility to infection with another virus or bacteria, and/or a pregnancy-related complication.

In one aspect, the present disclosure provides a method of preventing infection by SARS-CoV, SARS-COV-2, and/or another coronavirus optionally selected from the group consisting of MERS-COV, HCoV-HKU1, HCoV-OC43, HCoV-229E, and HCoV-NL63 in a subject in need thereof, comprising administering to the subject a prophylactically effective amount of an antibody, or antigen-binding fragment thereof, a CAR or an ADC as described herein.

In one aspect, the present disclosure provides a method of inducing an immune response against SARS-COV, SARS-COV-2, and/or another coronavirus optionally selected from the group consisting of MERS-COV, HCoV-HKU1, HCoV-OC43, HCoV-229E, and HCoV-NL63 in a subject in need thereof, comprising administering at least one antibody, or antigen-binding fragment thereof, a CAR or an ADC as described herein.

In some embodiments, the immune response elicits immunoprotection against SARS-COV, SARS-COV-2 and/or another coronavirus.

In one aspect, the present disclosure provides a method of inhibiting or blocking infection of susceptible cells by SARS-COV, SARS-COV-2, and/or another coronavirus optionally selected from the group consisting of MERS-COV, HCoV-HKU1, HCoV-OC43, HCoV-229E, and HCoV-NL63 in a subject in need thereof, comprising administering at least one antibody, or antigen-binding fragment thereof, a CAR or an ADC as described herein.

In one aspect, provided herein is a method of preventing the need for a subject infected with SARS-COV, SARS-COV-2, and/or another coronavirus optionally selected from the group consisting of MERS-COV, HCoV-HKU1, HCoV-OC43, HCoV-229E, and HCoV-NL63 to be placed on a ventilator, or reducing the time that a subject infected with SARS-COV or SARS-COV-2 or another coronavirus optionally selected from the group consisting of MERS-COV, HCoV-HKU1, HCoV-OC43, HCoV-229E, and HCoV-NL63 is on a ventilator, comprising administering to the subject a prophylactically or therapeutically effective amount of an antibody, or antigen-binding fragment thereof, a CAR or an ADC as described herein.

In one aspect, provided herein is a method of preventing the onset of pneumonia in a subject infected SARS-COV, SARS-COV-2, and/or another coronavirus optionally selected from the group consisting of MERS-COV, HCoV-HKU1, HCoV-OC43, HCoV-229E, and HCoV-NL63, or treating pneumonia and/or the symptoms of pneumonia in a subject for a subject infected SARS-COV or SARS-COV-2 or another coronavirus optionally selected from the group consisting of MERS-COV, HCoV-HKU1, HCoV-OC43, HCoV-229E, and HCoV-NL63, comprising administering to the subject a prophylactically or therapeutically effective amount of an antibody, or antigen-binding fragment thereof, a CAR or an ADC as described herein.

In some embodiments, the subject is a human subject.

In some embodiments, the subject is immunocompromised. In some embodiments, the subject is at risk of exposure to SARS-COV, SARS-COV-2, and/or another coronavirus. In some embodiments, the subject has at least one risk factor which renders them more prone to a poor clinical outcome. In some embodiments, the at least one risk factor is one or more of (i) an old age such as over 55, 60 or 65 years old, (ii) diabetes, (iii) a chronic respiratory condition such as asthma, cystic fibrosis, another fibrotic condition, and COPD, (iv) obesity, (iv) hypertension, (v) a cardiac or cardiovascular condition, such as heart defects or abnormalities, (vi) a chronic inflammatory or autoimmune condition such as lupus and multiple sclerosis, and (vii) an immunocompromised status which may be caused by cancer, undergoing chemotherapy, smoking, bone marrow or organ transplantation, immune deficiencies, poorly controlled HIV infection or AIDS, or prolonged use of corticosteroids or other immunosuppressive medications.

In some embodiments, the antibody, or antigen-binding fragment thereof, is administered in combination with an additional at least one anti-CoV-S antibody or antigen-binding fragment. In one embodiment, the administration is at the same time. In one embodiment, the administration is sequential. In some embodiments, the additional anti-CoV-S antibody or antigen-binding fragment is adintrevimab.

In some embodiments, the antibody, or antigen-binding fragment thereof, is administered as a single dose.

In some embodiments, the antibody, or antigen-binding fragment thereof, is administered intravenously. In one embodiment, the antibody, or antigen-binding fragment thereof, is administered via an intravenous (IV) push. In another embodiment, the antibody, or antigen-binding fragment thereof, is administered via an IV bolus. In another embodiment, the antibody, or antigen-binding fragment thereof, is administered via IV infusion. In other embodiments, the antibody, or antigen-binding fragment thereof, is administered intramuscularly.

In some embodiments, the antibody, or antigen-binding fragment thereof, is administered at a dose of about 100 mg to 5000 mg, about 100 mg to 4500 mg, about 100 mg to 4000 mg, about 100 mg to about 3500 mg, about 100 mg to about 3000 mg, about 100 mg to about 2500 mg, about 100 mg to about 2000 mg, about 200 mg to about 1500 mg, about 300 mg to about 600 mg, about 500 mg to about 1200 mg, about 300 mg to about 1200 mg, about 500 to about 1000 mg, about 1000 mg to about 1500 mg, about 1500 mg to about 2000 mg, about 2000 mg to about 2500 mg, about 2500 mg to about 3000 mg, about 3000 mg to about 3500 mg, about 3500 mg to about 4000 mg, about 4000 to about 4500 mg, or about 4500 mg to about 5000 mg. In some embodiments, the antibody, or antigen-binding fragment thereof, is administered at a dose of about 200 mg, about 300 mg, about 400 mg, about 500 mg, about 600 mg, about 700 mg, about 800 mg, about 900 mg, about 1000 mg about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, about 3600 mg, about 3700 mg, about 3800 mg, about 3900 mg, about 4000 mg, about 4100 mg, about 4200 mg, about 4300 mg, about 4400 mg, about 4500 mg, about 4600 mg, about 4700 mg, about 4800 mg, about 4900 mg, or about 5000 mg.

In one embodiment, the antibody, or antigen-binding fragment thereof, is administered once. In one embodiment, the antibody, or antigen-binding fragment thereof, is administered twice. In one embodiment, the antibody, or antigen-binding fragment thereof, is administered weekly. In another embodiment, the antibody, or antigen-binding fragment thereof, is administered daily, weekly, every two weeks, monthly, or every two months. In one embodiment, the antibody, or antigen-binding fragment thereof, is administered weekly for about four weeks, once weekly for about a month, weekly for about 5 weeks, weekly for about 6 weeks, weekly for about 7 weeks, or weekly for about two months, or weekly for about three months.

In one embodiment, the antibody, or antigen-binding fragment thereof, is administered monthly for about one month, monthly for about two months, monthly for about three months, or monthly for about four months. In one embodiment, the antibody, or antigen-binding fragment thereof, is administered once, or multiple times, e.g., twice, three times, four times, or five times, for the first one month, the first two months, the first three months, the first four months, or the first five months.

In one embodiment, the antibody, or antigen-binding fragment thereof, is administered to a recipient subject with a frequency of once every twenty-six weeks or less, such as once every sixteen weeks or less, once every eight weeks or less, once every four weeks or less, once every two weeks or less, once every week or less, or once daily or less.

The antibody, or antigen-binding fragment thereof, may be administered every year or less, every 6 moths or less, every three months or less, every one month or less, every two weeks or less, every week or less, once daily or less, multiple times per day, and/or every few hours. In one embodiment, the administration is given every month, every 2 months, every 3 months, every 4 months, every 5 months, every 6 months, every 7 months, every eight months, every 9 months, every 10 months, every 11 months, or once a year.

In some embodiments, the antibody, or antigen-binding fragment thereof, is administered in combination with one or more additional anti-CoV-S-antibodies.

In one aspect, the present disclosure provides a method of producing the antibody, or antigen-binding portion thereof, as described herein, the method comprising expressing the antibody, or antigen-binding portion thereof, in a recombinant cell, and isolating the antibody, or antigen-binding portion thereof, from the cell. In some embodiments, the method further comprises formulating the antibody, or antigen-binding portion thereof, isolated from the cell into a pharmaceutical composition.

In another aspect, the present disclosure provides a library of antibodies comprising at least 2, 3, 4, 5, 6, 7, 8, 9, 10 or more isolated antibodies, or antigen-binding fragments thereof, that bind to the spike protein of a corona virus (CoV-S), wherein said antibodies, or antigen-binding fragments thereof, each comprise a heavy chain variable region (VH) and a light chain variable region (VL), wherein each VH consists of an amino acid sequence selected from the group consisting of any one of the VH sequences in Tables 3 and 5, and wherein each VL consists of an amino acid sequence selected from the group consisting of any one of the VL sequences in Tables 4 and 6.

In one aspect, the present disclosure provides a composition comprising two or more isolated antibodies, or antigen-binding fragment thereof, wherein said antibodies, or antigen-binding fragments thereof, each comprise a heavy chain variable region (VH) and a light chain variable region (VL), wherein each VH consists of an amino acid sequence selected from the group consisting of any one of the VH sequences in Tables 3 and 5, and wherein each VL consists of an amino acid sequence selected from the group consisting of any one of the VL sequences in Tables 4 and 6.

In another aspect, the present disclosure provides a composition comprising two or more isolated antibodies, or antigen-binding fragments thereof, selected from the group consisting of VYD223, VYD224, and VYD225.

In some embodiments, the composition comprises VYD224 and VYD225.

In one aspect, the present disclosure provides a pharmaceutical composition comprising a composition described herein, and a pharmaceutically acceptable carrier or excipient.

In another aspect, the present disclosure provides a method of treating infection by SARS-CoV, SARS-COV-2, and/or another coronavirus optionally selected from the group consisting of MERS-COV, HCoV-HKU1, HCoV-OC43, HCoV-229E, and HCoV-NL63, or treating a condition, symptom, disease, or disorder associated with said infection in a subject in need thereof, comprising administering to the subject a therapeutically effective amount of the composition or the pharmaceutical composition described herein.

In some embodiments, the condition, symptom, disease, or disorder comprises at least one of bronchitis, pneumonia, respiratory failure, acute respiratory failure, organ failure, multi-organ system failure, pediatric inflammatory multisystem syndrome, acute respiratory distress syndrome, blood clot, a cardiac condition, myocardial injury, myocarditis, heart failure, cardiac arrest, acute myocardial infarction, dysrhythmia, venous thromboembolism, post-intensive care syndrome, shock, anaphylactic shock, cytokine release syndrome, septic shock, disseminated intravascular coagulation, ischemic stroke, intracerebral hemorrhage, microangiopathic thrombosis, psychosis, seizure, nonconvulsive status epilepticus, traumatic brain injury, stroke, anoxic brain injury, encephalitis, posterior reversible leukoencephalopathy, necrotizing encephalopathy, post-infectious encephalitis, autoimmune mediated encephalitis, acute disseminated encephalomyelitis, acute kidney injury, acute liver injury, pancreatic injury, immune thrombocytopenia, subacute thyroiditis, a gastrointestinal complication, aspergillosis, increased susceptibility to infection with another virus or bacteria, and/or a pregnancy-related complication.

In one aspect, the present disclosure provides a method of preventing infection by SARS-CoV, SARS-COV-2, and/or another coronavirus optionally selected from the group consisting of MERS-COV, HCoV-HKU1, HCoV-OC43, HCoV-229E, and HCoV-NL63 in a subject in need thereof, comprising administering to the subject a prophylactically effective amount of the composition or the pharmaceutical composition as described herein.

In one aspect, the present disclosure provides a method of inducing an immune response against SARS-COV, SARS-COV-2, and/or another coronavirus optionally selected from the group consisting of MERS-COV, HCoV-HKU1, HCoV-OC43, HCoV-229E, and HCoV-NL63 in a subject in need thereof, comprising administering the composition or the pharmaceutical composition of the present disclosure as described herein.

In some embodiments, the immune response elicits immunoprotection against SARS-COV, SARS-COV-2 and/or another coronavirus.

In another aspect, the present disclosure provides a method of inhibiting or blocking infection of susceptible cells by SARS-COV, SARS-COV-2, and/or another coronavirus optionally selected from the group consisting of MERS-COV, HCoV-HKU1, HCoV-OC43, HCoV-229E, and HCoV-NL63 in a subject in need thereof, comprising administering the composition or the pharmaceutical composition of the present disclosure as described herein.

In one aspect, the present disclosure provides a method of preventing the need for a subject infected with SARS-COV, SARS-COV-2, and/or another coronavirus optionally selected from the group consisting of MERS-COV, HCoV-HKU1, HCoV-OC43, HCoV-229E, and HCoV-NL63 to be placed on a ventilator, or reducing the time that a subject infected with SARS-COV or SARS-COV-2 or another coronavirus optionally selected from the group consisting of MERS-COV, HCoV-HKU1, HCoV-OC43, HCoV-229E, and HCoV-NL63 is on a ventilator, comprising administering to the subject a prophylactically or therapeutically effective amount of the composition or the pharmaceutical composition of the present disclosure as described herein.

In another aspect, the present disclosure provides a method of preventing the onset of pneumonia in a subject infected SARS-COV, SARS-COV-2, and/or another coronavirus optionally selected from the group consisting of MERS-COV, HCoV-HKU1, HCoV-OC43, HCoV-229E, and HCoV-NL63, or treating pneumonia and/or the symptoms of pneumonia in a subject for a subject infected SARS-COV or SARS-COV-2 or another coronavirus optionally selected from the group consisting of MERS-COV, HCoV-HKU1, HCoV-OC43, HCoV-229E, and HCoV-NL63, comprising administering to the subject a prophylactically or therapeutically effective amount of the composition or the pharmaceutical composition of the present disclosure as described herein.

In some embodiments, the subject is a human subject.

In some embodiments, the subject is immunocompromised.

In some embodiments, the subject is at risk of exposure to SARS-COV, SARS-COV-2, and/or another coronavirus.

In some embodiments, the subject has at least one risk factor which renders them more prone to a poor clinical outcome.

In some embodiments, the at least one risk factor is one or more of (i) an old age such as over 55, 60 or 65 years old, (ii) diabetes, (iii) a chronic respiratory condition such as asthma, cystic fibrosis, another fibrotic condition, and COPD, (iv) obesity, (iv) hypertension, (v) a cardiac or cardiovascular condition, such as heart defects or abnormalities, (vi) a chronic inflammatory or autoimmune condition such as lupus and multiple sclerosis, and (vii) an immunocompromised status which may be caused by cancer, undergoing chemotherapy, smoking, bone marrow or organ transplantation, immune deficiencies, poorly controlled HIV infection or AIDS, or prolonged use of corticosteroids or other immunosuppressive medications.

In some embodiments, the composition is administered intravenously or intramuscularly. In one embodiment, the composition is administered via an IV push. In another embodiment, the composition, is administered via an IV bolus.

In some embodiments, the composition is administered at a dose of about 100 mg to about 5000 mg, about 100 mg to 4500 mg, about 100 mg to 4000 mg, about 100 mg to about 3500 mg, about 100 mg to about 3000 mg, about 100 mg to about 2500 mg, about 100 mg to about 2000 mg, about 200 mg to about 1500 mg, about 300 mg to about 600 mg, about 500 mg to about 1200 mg, about 300 mg to about 1200 mg, about 500 to about 1000 mg, about 1000 mg to about 1500 mg, about 1500 mg to about 2000 mg, about 2000 mg to about 2500 mg, about 2500 mg to about 3000 mg, about 3000 mg to about 3500 mg, about 3500 mg to about 4000 mg, about 4000 to about 4500 mg, or about 4500 mg to about 5000 mg.

In some embodiments, the composition is administered at a dose of about 300 mg, about 500 mg, about 600 mg, about 1000 mg, about 1200 mg, about 1500 mg, about 2000 mg, about 2500 mg, about 300 mg, about 3500 mg, about 4000 mg, about 4500 mg, or about 5000 mg.

In some embodiments, the composition is administered once, or is administered weekly, monthly, every two months, every three months, or every six months.

In some embodiments, administering the composition results in about 30%, about 40%, about 50%, about 60%, or about 70% relative risk reduction, e.g., for at least 2 months, at least 4 months, at least 6 months, at least 8 months, at least 10 months, or at least 12 months.

In another aspect, the present invention provides an isolated nucleic acid molecule, e.g., a DNA or an mRNA molecule, encoding the antibody, or antigen-binding fragment thereof, of the present invention, as described herein.

In yet another aspect, the present invention provides an isolated mRNA molecule encoding the antibody, or antigen-binding fragment thereof, of the invention, as described herein.

In one aspect, the present invention provides a composition comprising the isolated nucleic acid molecule, or the isolated mRNA molecule of the invention, as described herein.

In another aspect, the present invention provides a kit comprising the antibody, or antigen-binding fragment thereof, the isolated nucleic acid molecule, or the isolated mRNA molecule, of the invention, as described herein, and instructions for use.

In yet another aspect, the present invention provides a vial comprising the antibody, or antigen-binding fragment thereof, the isolated nucleic acid molecule, or the isolated mRNA molecule, of the invention, as described herein.

In some embodiments, the vial is about 1 mL, about 2 mL, about 4 ml, about 8 mL, about 12 mL, about 16 mL, about 20 ml, or about 24 mL in volume.

In some embodiments, each vial comprises about 100 mg, about 200 mg, about 300 mg, about 500 mg, about 600 mg, about 700 mg, about 800 mg, about 900 mg, about 1000 mg, about 1500 mg, about 2000 mg, or about 2500 mg of the antibody or antigen-binding fragment thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1G depict the serum binding and neutralizing activity following BA.1 breakthrough infection. FIG. 1A: Vaccination, infection, and blood draw timelines. FIGS. 1B-1C: Serum IgG (FIG. 1B) and (FIG. 1C) IgA reactivity with recombinant WT and BA.1 Hexapro-stabilized S proteins (left) and RBDs (right) following BA.1 breakthrough infection. Serum samples from uninfected/vaccinated donors at one month (1M) or six months (6M) following primary vaccination (2× mRNA) or one month following booster mRNA vaccination (3× mRNA) are shown for comparison. The fold change in median EC₅₀ against BA.1 relative to D614G is shown above each paired set of measurements. Black bars represent median binding EC₅₀ titers. Dotted lines represent the lower limit of detection. FIGS. 1D-G: Serum neutralizing activity against SARS-COV-2 D614G, Beta, Delta, and BA.1 and SARS-COV (FIG. 1D) one month after primary mRNA vaccination (n=12), (FIG. 1E) six months after primary mRNA vaccination (n=10), (FIG. 1F) one month after booster mRNA vaccination (n=11), and (FIG. 1G) 14 to 27 days after BA.1 breakthrough infection (n=7), as measured using a murine leukemia virus (MLV)-based pseudovirus neutralization assay. Plotted values represent serum neutralizing IC₅₀ titers and values shown above the data points indicate the median IC₅₀ titer. The fold change in IC₅₀ titer for each virus relative to D614G is shown in parentheses. Donors infected after primary mRNA vaccination are shown as circles and those infected after booster mRNA vaccination are shown as triangles. Statistical comparisons were determined by (FIGS. 1B-C) two-sided Kruskal-Wallis test with Dunn's multiple comparisons or (FIG. 1D) Friedman's test with multiple comparisons. 1M, one month; 6M, six months; EC₅₀, 50% effective concentration; IC₅₀, 50% inhibitory concentration; WT, wild type. *P<0.05, **P<0.01, ***P<0.001, ****P<0.0001.

FIGS. 2A-2H depict SARS-COV-2 S-specific B cell responses induced by BA.1 breakthrough infection. FIGS. 2A-2B. Frequency of circulating B cells that recognize recombinant WT and BA.1 RBDs among (FIG. 2A) IgG⁺ and (FIG. 2B) IgA⁺ B cells in BA.1 breakthrough infection donors and uninfected/vaccinated donors at one- or six-months following primary vaccination (2× mRNA) or one month following booster mRNA vaccination (3× mRNA), as measured by flow cytometry. Bars indicate median frequencies. Donors with breakthrough infections occurring after primary mRNA vaccination are shown as circles and those infected after booster mRNA vaccination are shown as triangles. FIG. 2C. Representative fluorescence-activated cell sorting (FACS) gating strategy used to identify RBD-directed B cells that are WT-specific or WT/BA.1 cross-reactive, shown for a pre-pandemic donor and a breakthrough infection donor. Percentages of WT-specific (tan) and WT/BA.1 cross-reactive (teal) B cells out of all RBD-reactive cells are shown in parentheses. FIGS. 2D-E. Mean proportions of RBD-reactive B cells that bind WT and/or BA.1 RBD among (FIG. 2D) total S⁺swIg⁺ B cells or (FIG. 2E) S⁺ swIg⁺ CD71+ B cells. Error bars represent standard errors of the mean. Samples collected six months following mRNA vaccination were excluded from this analysis due to low numbers of RBD-specific CD71+ cells at this time point. FIGS. 2F-H. Percentage of S-reactive swIg⁺ B cells that target the (FIG. 2F) NTD, (FIG. 2G) RBD, and (FIG. 2H) Hexapro-stabilized S2 subunits. Black bars represent median percentages. For breakthrough infection donors, this analysis was restricted to S⁺swIg⁺CD71⁺ B cells to capture the activated response. Statistical comparisons were determined by (FIGS. 2A-B) two-way ANOVA with subsequent Dunnett's multiple comparisons test or (FIGS. 2D-H) two-sided Kruskal-Wallis test with Dunn's multiple comparisons. 1M, one month; 6M, six months; swIg, class-switched immunoglobulin; WT, wild type. *P<0.05, **P<0.01, ***P<0.001, ****P<0.0001.

FIGS. 3A-3E depict sequence features of RBD-directed monoclonal antibodies isolated from BA.1 breakthrough infection donors. FIG. 3A. Proportion of antibodies that bind recombinant WT and/or BA.1 RBD antigens from each donor, as determined by biolayer interferometry (BLI). The number of antibodies isolated from each donor is indicated at the top of each bar. FIG. 3B. Clonal lineage analysis. Clonally expanded lineages are represented as colored slices proportional to the lineage size, and unique clones are combined and shown as a single grey segment. The total number of antibodies is shown in the center of each pie. FIG. 3C. Germline IGHV gene usage frequencies among anti-RBD antibodies derived from breakthrough infection donors. Germline gene frequency distributions among anti-RBD antibodies isolated from mRNA vaccinated donors and unselected (baseline) memory B cell repertoires are included for reference (17). FIG. 3D Distribution of HCDR3 amino acid lengths in BA.1-reactive antibodies. The dotted black line represents the median, and the lower and upper lines represent the first and third quartile, respectively. FIG. 3E. Number of VH nucleotide substitutions in antibodies isolated from each breakthrough infection donor, with medians shown by black bars. Statistical significance was determined by Fisher's exact test compared to the baseline repertoire. 1M, one month; CDR, complementarity determining region; IGHV, immunoglobulin heavy variable domain; VH, variable heavy chain; WT, wild type. ***P<0.001, ****P<0.0001.

FIGS. 4A-4G depict binding and neutralization properties of anti-RBD antibodies isolated following BA.1 breakthrough infection. FIG. 4A. Fab binding affinities for recombinant WT and BA.1 RBD antigens, as measured by BLI. Fabs with no detectable binding activity or with binding kinetics that could not be fit to a 1:1 binding model were excluded. FIG. 4B. Proportion of BA.1-reactive antibodies with the indicated binding affinities for SARS-COV-2 VOC RBDs and SARS-COV RBD, as measured by BLI. Antibodies with weak binding affinities that could not be fit to a 1:1 binding model are shown as >100 nM. FIG. 4C. Proportion of antibodies from each donor with the indicated levels of neutralizing activity against MLV-SARS-COV-2 D614G and BA.1 at a concentration of 5 g/ml. FIG. 4D. MLV-SARS-COV-2 D614G and BA.1 neutralization IC₅₀s for antibodies that displayed >90% neutralization against D614G and/or BA.1 at a concentration of 5 μg/ml. FIG. 4E. Heatmap showing neutralization potency and binding breadth of BA.1 neutralizing antibodies. The bottom bar shows convergent IGHV germline gene families. FIG. 4F. Pie charts showing convergent germline gene usage among BA.1-neutralizing antibodies (right) compared to the baseline human antibody repertoires (left) (17). FIG. 4G. Competitive binding profiles of BA.1 neutralizing antibodies utilizing convergent IGHV germline genes, as determined by BLI sandwich competition assay using ACE2 and the indicated comparator antibodies. Fab, antigen binding fragment; IC₅₀, 50% inhibitory concentration; IGHV, immunoglobulin gene heavy variable; K_{D}, equilibrium dissociation constant; N.B., non-binding; WT, wild type.

FIG. 5 depicts the prevalence of circulating SARS-COV-2 variants. Genomic sequencing analyses of SARS-COV-2 infections occurring in the United States CDC Region 1 (Connecticut, Maine, Massachusetts, New Hampshire, Rhode Island, and Vermont) are colored by the indicated Pango lineage.

FIGS. 6A-6C depict SARS-COV-2 RBD-specific B cell staining. FIG. 6A. Representative FACS gating strategy to determine frequencies of WT- and BA.1-RBD-reactive B cells among IgG⁺ and IgA⁺ B cells. FIG. 6B. Representative FACS gating strategy to determine the proportion of CD71⁺S⁺RBD⁺ cells that are WT-specific or BA.1/WT cross-reactive. FIG. 6C. Representative FACS gating strategy used to calculate the proportion S-specific B cells directed to the NTD, RBD, and S2 subdomains. FSC-A, forward scatter area; FSC-H, forward scatter height; SSC-A, side scatter area.

FIG. 7 depicts distribution of subdomain specificities within the S-specific B cell response following BA.1 breakthrough infection. Proportions of S-reactive CD71⁺ swIg⁺ B cells that recognize the NTD (left), RBD (middle), or prefusion-stabilized S2 (right) subdomains in donors who experienced breakthrough infection after either two-dose or three-dose mRNA vaccination, as determined by flow cytometry. Black bars indicate medians.

FIG. 8 depicts representative FACS gating strategy for single-cell sorting RBD-specific swIg⁺ B cells. FACS plots shown are gated on swIg⁺ CD19+ B cells. A pre-pandemic donor was included as a negative control.

FIG. 9 depicts binding reactivities of breakthrough infection-derived antibodies isolated from CD71+ B cells. The number at the top of each bar represents the total number of antibodies.

FIGS. 10A-10C depict antibody breadth of activity against SARS-COV-2 variants and SARS-CoV. FIG. 10A. Fab binding affinities of BA.1 neutralizing antibodies to SARS-COV RBD, as determined by BLI. FIG. 10B. Neutralization IC50s against SARS-COV and BA.1 among antibodies displaying Fab binding to SARS-COV. FIG. 10C. Neutralization potency (left) and Fab binding affinity (right) for the indicated SARS-COV-2 variants of concern and SARS-COV. IC50, 50% inhibitory concentration; KD, equilibrium dissociation constant; N.B., non-binding; n.d., not determined; N.N., non-neutralizing.

FIG. 11 depicts IGHV germline gene frequency distributions among BA.1 neutralizing antibodies. Frequency distributions of all RBD-binding antibodies isolated from breakthrough infection donors and the human baseline (unselected) repertoire are included for reference (17). Statistical comparisons were made by Fisher's exact test compared to the baseline repertoire. IGHV, immunoglobulin heavy variable domain. *P<0.05, **P<0.01, ***P<0.001, ****P<0.0001.

FIG. 12 depicts HCDR3 amino acid lengths of BA.1 neutralizing antibodies utilizing IGH3-53/3-66 germline genes, with baseline antibody repertoire lengths included for comparison (17). Statistical comparisons were determined by Kruskal-Wallis test with subsequent Dunn's multiple comparisons. HCDR3, heavy complementarity determining region 3. ****P<0.0001

FIG. 13 depicts neutralizing activity of antibodies utilizing convergent IGHV germline genes against D614G and BA.1, as determined using an MLV-based pseudovirus assay. Black bars represent median IC50s. Statistical significance was determined by two-way ANOVA with subsequent Dunnett's multiple comparisons test IC50, 50% inhibitory concentration. IGHV, immunoglobulin heavy variable domain. **P<0.01.

FIGS. 14A-14B depict sequence features of breakthrough infection-derived IGHV1-69 neutralizing antibodies. FIG. 14A. Light chain IGLV germline gene usage among non-ACE2-competitive IGHV1-19 antibodies. The total number of antibodies is shown in the center of the pie. FIG. 14B. Pairwise comparisons of LCDR3 amino acid identity. The percentage identity of each pairwise comparison is shown in the middle of each square. LCDR3, light chain complementarity determining region 3; IGKV, immunoglobulin kappa variable domain. IGLV, immunoglobulin lambda variable domain.

FIGS. 15A-15D depict the serum neutralizing antibody responses induced following BA.1 breakthrough infection. FIG. 15A. Timeline of vaccination, BA.1 breakthrough infection, and sample collections. FIG. 15B. Paired analysis of serum neutralizing activity against SARS-COV-2 D614G and BA.1, BA.2, BA.2.75, BA.4/5, Beta, and Delta variants, and SARS-COV (SARS1) at 1-month (T1) and 5-6-month (T2) time points, as determined via a MLV-based pseudovirus neutralization assay. Connected data points represent paired samples for each donor, and the median fold change in serum titer between the two time points is shown in parentheses. Dotted lines represent the lower limit of detection of the assay. FIG. 15C. Serum neutralizing titers against SARS-COV-2 variants and SARS-CoV in samples collected at (left) 1-month and (right) 5-6-month post-breakthrough infection for each donor. Median titers are shown above the data points. Dotted lines represent the lower limit of detection of the assay. FIG. 15D. Fold change in serum neutralizing titers for the indicated SARS-COV-2 variants and SARS-COV relative to SARS-COV-2 D614G at early (T1) and late (T2) time points. Black bars represent median fold changes. Dotted line indicates no change in IC₅₀. Breakthrough infection donors infected after two-dose mRNA vaccination (n=4) are shown as circles and those infected after a third mRNA dose (n=3) are shown as triangles. One two-dose vaccinated breakthrough donor was lost to follow-up at the second time point. Statistical comparisons were determined by (FIG. 15B) Wilcoxon matched-pairs signed rank test, (FIG. 15C) Friedman's one-way ANOVA with Dunn's multiple comparisons, or (FIG. 15D) mixed model ANOVA. *P<0.05; **P<0.01; ****P<0.0001; ns, not significant.

FIGS. 16A-16F depict the SARS-COV-2 RBD-specific memory B cell responses following BA.1 breakthrough infection. FIG. 16A. Representative fluorescence-activated cell sorting gating strategy used to enumerate frequencies of (top) total (WT+BA.1) RBD-reactive B cells among class-switched (IgG⁺ or IgA⁺) CD19+ B cells and (bottom) WT-specific, BA.1-specific, and WT/BA.1 cross-reactive B cells among total RBD-reactive, class-switched (IgG⁺ or IgA⁺) CD19⁺ B cells. FIGS. 16B-C. Frequencies of (B) total RBD-reactive or (C) WT/BA.1 RBD cross-reactive B cells among class-switched CD19⁺ B cells at 1-month (T1) and 5-6-month (T2) time points. Connected data points represent paired samples for each donor. Donors infected after two-dose mRNA vaccination (n=4) are shown as circles and those infected after a third mRNA dose (n=3) are shown as triangles. One two-dose vaccinated breakthrough donor was censored at the second time point. FIG. 16D. Mean proportions of RBD-reactive, class-switched B cells that display WT-specific, BA.1-specific or WT/BA.1-cross-reactive binding at each time point. Error bars indicate standard error of mean. FIG. 16E. Clonal lineage analysis of RBD-directed antibodies isolated from four donors at the early (T1) and late (T2) time points. Clonally expanded lineages (defined as antibodies with the same heavy and light chain germlines, same CDR3 lengths, and ≥80% CDRH3 sequence identity) are represented as colored slices. Each colored slice represents a clonal lineage with the size of the slice proportional to the lineage size. Unique clones are combined into a single gray segment. The number of antibodies is shown in the center of each pie. Three of the donors (IML4042, IML4043, and IML4044) experienced BA.1 breakthrough infection following two-dose mRNA vaccination and the remaining donor (IML4045) was infected after a booster immunization. FIG. 16F. Levels of somatic hypermutation, as determined by the number of nucleotide substitutions in the variable heavy (VH) region, at the early and late time points among WT-specific, WT/BA.1 cross-reactive, and BA.1-specific antibodies. Medians are shown by black bars. Statistical significance was determined by (FIGS. 16B and C) Wilcoxon matched-pairs signed rank test or (FIGS. 16D and F) Mann-Whitney U test. swIg⁺, class-switched immunoglobulin. PE, phycoerythrin; *P<0.05; **P<0.01.

FIGS. 17A-17F depicts the binding and neutralizing properties of RBD-directed antibodies induced by BA.1 breakthrough infection. FIGS. 17A-B. Fab binding affinities of WT/BA.1 cross-reactive antibodies for recombinant WT and BA.1 RBD antigens, as measured by BLI, are plotted as bivariates for antibodies derived from (left) 1-month and (right) 5-6-month time points in (A) and summarized as a column dot plot in (B). Median affinities are indicated by black bars and shown below data points. FIG. 17C. Proportions of WT/BA.1 cross-reactive antibodies at each time point that show increased affinity for the BA.1 RBD relative to WT (red shades) or increased affinity for WT RBD (blue shades). Values represent the percentage of antibodies belonging to each of the indicated categories. FIGS. 17D-E. Neutralizing activities of cross-binding antibodies against SARS-COV-2 D614G and BA.1, as determined by an MLV-based pseudovirus neutralization assay. IC₅₀ values are plotted in (D) as bivariates for antibodies isolated from (left) 1-month and (right) 5-6-month tie points and summarized as column dot plots in (E). Median IC₅₀ values are indicated by black bars and shown below data points. FIG. 17F. Proportions of WT/BA.1 cross-neutralizing antibodies at each time point that show increased neutralizing potency against BA.1 (red shades) or D614G (blue shades). Values represent the percentage of antibodies belonging to each of the indicated categories. Statistical comparisons were determined by (B and E) Kruskal-Wallis test with subsequent Dunn's multiple comparisons or (C and F) Mann-Whitney U test. IC₅₀, 50% inhibitory concentration; K_{D}, equilibrium dissociation constant; *P<0.05; **P<0.01; ****P<0.0001.

FIGS. 18A-18G depict the breadth of D614G/BA.1 cross-neutralizing antibodies at early and late time points following BA.1 breakthrough infection. FIG. 18A. Fab binding affinities of D614G/BA.1 cross-neutralizing antibodies isolated at 1-month (T1) and 5-6-month (T2) time points for recombinant SARS-COV-2 variant RBDs and the SARS-COV RBD, as determined by BLI. Black bars represent medians. FIG. 18B. Pie charts showing the proportions of antibodies derived from (left) early and (right) late time points that bound the indicated number of SARS-COV-2 variant RBDs with Fab K_{D}s≤10 nM. The total number of antibodies is shown in the center of each pie. FIG. 18C. Proportions of D614G/BA.1 cross-neutralizing antibodies with the indicated fold changes in Fab binding affinities for recombinant SARS-COV-2 variant RBDs relative to the WT RBD. FIG. 18D. Pie charts showing frequencies of the indicated convergent germline genes among D614G/BA.1 cross-neutralizing antibodies isolated at early (T1) and late (T2) timelines. Germline gene frequencies observed in baseline human antibody repertoires (upper right) are shown for comparison. FIG. 18E. Structural projections of binding escape mutations determined for the indicated convergent antibodies using deep mutational scanning analysis of yeast-displayed SARS-COV-2 BA.1 RBD mutant libraries. The RBD surface is colored by a gradient ranging from no escape (white) to strong escape (red) at each site. FIG. 18F. Heatmap summarizing convergent antibody escape mutations present in the indicated SARS-COV-2 Omicron sub-lineages. FIG. 18G. Fab binding affinities of convergent antibodies utilizing the indicated germline genes for SARS-COV-2 WT and Omicron sub-variant RBD antigens, as measured by BLI. Black bars indicate median affinities. Statistical comparisons were determined by (A and C) Kruskal-Wallis test with Holms corrected multiple pairwise comparisons, (B and D) Fisher's exact test, or (G) Kruskal-Wallis test with subsequent Dunn's multiple comparisons with WT. K_{D}, equilibrium dissociation constant; *P<0.05; **P<0.01; ***P<0.001; ****P<0.0001.

FIGS. 19A-19B depict SARS-COV-2 antigen-specific B cell staining and sorting. FIG. 19A. Representative FACS gating strategy to determine frequencies of WT and/or BA.1 RBD-reactive B cells among class-switched (IgG⁺ or IgA⁺) B cells. The frequency of events in each gate relative to the parent gate are shown as percentages in each plot. FIG. 19B. FACS gates used for single-cell sorting of WT and/or Omicron BA.1 RBD-specific memory B cells in 4 individuals 5-6 months following BA.1 breakthrough infection, Donors IML4042, IML4043, and IML4044 experienced breakthrough infection following two-dose mRNA vaccination, and IML4045 was infected after a third mRNA dose. A healthy pre-pandemic donor sample is shown as a control. FSC-A, forward scatter area; FSC-H, forward scatter height; swIg⁺, class-switched immunoglobulin; SSC-A, side scatter area.

FIG. 20 depicts the cross-reactivity of RBD-directed B cells at early and late time points following BA.1 breakthrough infection. Proportion of RBD-directed class-switched B cells that are (left) WT-specific, (middle) WT/BA.1 cross-reactive, and (right) BA.1-specific at 1-month (T1) and 5-6-month (T2) time points, as determined by flow cytometry. Donors infected after two-dose mRNA vaccination (n=4) are shown as circles and those infected after a third mRNA booster dose (n=3) are shown as triangles. One two-dose vaccinated breakthrough donor was censored at the second time point. Statistical comparisons were determined by Mann-Whitney U tests. **P<0.01.

FIGS. 21A-21B depict the cross-reactivity of RBD-directed monoclonal antibodies isolated at 1- and 5-6 months following BA.1 breakthrough infection. FIG. 21A. Proportion of BA.1-specific, WT-specific, and WT/BA.1 cross-reactive antibodies isolated from breakthrough infection donors at 1-month (T1) and 5-6-month (T2) time points, as determined by BLI. FIG. 21B. Summary of antibody cross-reactivity at both time points. Connected data points represent paired samples for each donor. Donors infected after two-dose mRNA vaccination (n=4) are shown as circles and those infected after a third mRNA booster dose (n=3) are shown as triangles. One two-dose vaccinated breakthrough donor was censored at the second time point.

FIG. 22 depicts the IGHV germline usage among cross-reactive antibodies. Human IGHV germline gene usage frequencies among WT/BA.1 cross-reactive antibodies at 1 month (T1) and 5-6 month (T2) time points. Germline gene distribution of RBD-directed antibodies derived from two-dose mRNA-vaccinated/uninfected donors were obtained from the CoV-AbDab database (29). Human baseline (unselected) repertoire frequencies were included for reference. Statistical comparisons were made by Fisher's exact test compared to the baseline repertoire. IGHV, immunoglobulin heavy variable domain. *P<0.05, **P<0.01, ***P<0.001, ****P<0.0001.

FIG. 23 depicts the binding and neutralization properties of BA. 1-specific antibodies. Heatmap showing neutralization IC₅₀s and SARS-COV-2 variant RBD binding affinities of BA.1-specific antibodies.

FIG. 24 depicts the binding breadth of D614G/BA.1 cross-neutralizing antibodies. Heatmap showing neutralization IC₅₀s and SARS-COV-2 variant RBD binding affinities of D614G/BA.1 cross-neutralizing antibodies isolated 5-6 months following BA.1 breakthrough infection. Antibodies utilizing convergent germline are indicated in the right-most column.

FIGS. 25A-25B depict the germline gene usage of D614G/BA.1 cross-neutralizing antibodies isolated 5-6 months following BA.1 breakthrough infection. FIG. 25A. Human IGHV germline distribution frequencies among D614G/BA.1 cross-neutralizing antibodies isolated 1-month (T1) and 5-6-months (T2) following breakthrough infection, with human baseline repertoire frequencies shown for comparison. FIG. 25B. Pie charts showing the proportion of cross-neutralizing antibodies isolated from each donor that utilize convergent germline genes. The total number of antibodies isolated from each donor is indicated above each pie chart. IGHV, immunoglobulin human variable domain; *P<0.05, **P<0.01, ***P<0.001, ****P<0.0001.

FIGS. 26A-26C depict the sequence and binding features of antibodies utilizing convergent germline genes. FIG. 26A. Pie charts showing light chain germline usage among IGHV1-69 antibodies isolated at 1-month (T1) and 5-6 month (T2) time points. The number of antibodies analyzed from each time point is indicated in the center of each pie. FIG. 26B. HCDR3 amino acid length distribution of IGHV3-53 and IGHV3-66 cross-neutralizing antibodies isolated 1-month (T1) and 5-6 months (T2) following BA.1 breakthrough infection. HCDR3 lengths of IGHV3-53/3-66-utilizing antibodies isolated following primary D614G infection and the baseline human antibody repertoire were included for comparison. FIG. 26C. Proportion of cross-neutralizing antibodies utilizing the indicated germline genes that compete with the ACE2 receptor for binding, as determined by a BLI competition assay. The number of antibodies analyzed is shown in the center of each pie. Statistical comparisons were determined by Kruskal-Wallis test with subsequent Dunn's multiple comparisons. A.A., amino acids; *P<0.05; ****P<0.0001.

FIGS. 27A-27C depict the deep mutational scanning analysis. FIG. 27A. Representative FACS gates used to select antibody-escape mutations in yeast-displayed Omicron BA.1 mutant libraries. Gates were drawn to capture ˜50% of wildtype Omicron BA.1-expressing yeast labeled at an antibody concentration 0.1× the selection concentration. From duplicate mutant libraries, yeast cells in the antibody-escape bin were sorted and sequenced. Post-sort mutant frequencies were compared to the pre-sort population to calculate per-mutant “escape fractions”, the fraction of cells expressing a mutation that were found in the antibody-escape sort gate. FIG. 27B. Correlation in per-mutation (left) and per-site (right) escape fractions in replica the library selections for each antibody. FIG. 27C. Lineplots at left show the total site-wise escape at each RBD site. This metric is mapped to structure in FIG. 17E. Sites of strong escape indicated by pink bars are shown at the mutation level in logoplots at center. Mutations are colored by their effects on ACE2 binding (scale bar at right). Note that prominent escape mutations such as K356T and I468N introduce N-linked glycosylation motifs.

FIG. 28 depicts the charge variant profile of VYD223 (also known as ADI-75865), the intermediate antibody (VYD223+K74T), and VYD225 (VYD223+K74T+N31Q) as determined by capillary isoelectric focusing (cIEF).

FIG. 29 depicts the intact masses observed for VYD223, the intermediate antibody (VYD223+K74T), and VYD225 (VYD223+K74T+N31Q) as determined by reduced deglycosylated liquid chromatography-mass spectrometry (LC-MS) of the heavy chain.

FIG. 30A depicts the level of monomer species by size exclusion chromatography (SEC) following protein A purification, the hydrophobic interaction (HIC) retention time, and the polyspecificity reagent (PSR) score of VYD223, and the intermediate antibody (VYD223+K74T). FIG. 30B depicts the binding affinity of VYD223 and the intermediate antibody (VYD223+K74T) against the RBD of the wild type SARS-COV-2 and the BA.2.75 variant.

FIG. 31 depicts the binding affinity of VYD225 and VYD223 against the RBD of SARS-COV-2 variants (the BA.1.1 variant, the BA.2.75 variant, the BQ.1.1 variant and the XBB variant).

FIG. 32 is a heatmap depicting the neutralization IC₅₀ of VYD225, VYD223, VYD224 (also known as ADI-80707), and adintrevimab against the wild type SARS-COV-2 strain, and the Omicron variants (i.e., the BF.7 variant, the BQ.1.1 variant, the BA.2.75 variant, the XBB.1 variant, and the XBB.1.5 variant) in a pseduovirus neutralization assay. VYD223-A has a K74T substitution in the VH chain of VYD223.

FIG. 33 depicts the biophysical analysis of VYD223 and VYD225.

FIG. 34 depicts the dose-response curve for VYD223 neutralization against SARS-COV-2 pseudoviruses.

### DETAILED DESCRIPTION

### A. Definitions

It is to be understood that this invention is not limited to the particular methodology, protocols, cell lines, animal species or genera, and reagents described, as such may vary. It is also to be understood that the terminology used herein is for the purpose of describing particular embodiments only and is not intended to limit the scope of the present invention, which will be limited only by the appended claims. As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

Spike protein (S protein): As used herein, unless stated otherwise S protein includes any coronavirus form of S protein. The term coronavirus S protein (“CoV-S”) is used to describe the S protein of any coronaviruses. In particular, the “SARS-COV-S” and “SARS-COV-2-S” encompass the following S protein of SARS-COV and of SARS-COV-2 amino acid sequences:

SARS-COV-S:
(SEQ ID NO: 1)
MFVFLVLLPLVSSQCVNLTTRTQLPPAYTNSFTRGVYYPDKVFRSSVLHSTQDLFLPFFSNVT
WFHAIHVSGTNGTKRFDNPVLPFNDGVYFASTEKSNIIRGWIFGTTLDSKTQSLLIVNNATNV
VIKVCEFQFCNDPFLGVYYHKNNKSWMESEFRVYSSANNCTFEYVSQPFLMDLEGKQGNFK
NLREFVFKNIDGYFKIYSKHTPINLVRDLPQGFSALEPLVDLPIGINITRFQTLLALHRSYLTPG
DSSSGWTAGAAAYYVGYLQPRTFLLKYNENGTITDAVDCALDPLSETKCTLKSFTVEKGIYQ
TSNFRVQPTESIVRFPNITNLCPFGEVENATRFASVYAWNRKRISNCVADYSVLYNSASFSTFK
CYGVSPTKLNDLCFTNVYADSFVIRGDEVRQIAPGQTGKIADYNYKLPDDFTGCVIAWNSNN
LDSKVGGNYNYLYRLFRKSNLKPFERDISTEIYQAGSTPCNGVEGENCYFPLQSYGFQPTNGV
GYQPYRVVVLSFELLHAPATVCGPKKSTNLVKNKCVNFNFNGLTGTGVLTESNKKFLPFQQF
GRDIADTTDAVRDPQTLEILDITPCSFGGVSVITPGTNTSNQVAVLYQDVNCTEVPVAIHADQ
LTPTWRVYSTGSNVFQTRAGCLIGAEHVNNSYECDIPIGAGICASYQTQTNSPGSASSVASQSI
IAYTMSLGAENSVAYSNNSIAIPTNFTISVTTEILPVSMTKTSVDCTMYICGDSTECSNLLLQY
GSFCTQLNRALTGIAVEQDKNTQEVFAQVKQIYKTPPIKDFGGFNFSQILPDPSKPSKRSFIEDL
LFNKVTLADAGFIKQYGDCLGDIAARDLICAQKFNGLTVLPPLLTDEMIAQYTSALLAGTITS
GWTFGAGAALQIPFAMQMAYRFNGIGVTQNVLYENQKLIANQFNSAIGKIQDSLSSTASALG
KLQDVVNQNAQALNTLVKQLSSNFGAISSVLNDILSRLDPPEAEVQIDRLITGRLQSLQTYVT
QQLIRAAEIRASANLAATKMSECVLGQSKRVDFCGKGYHLMSFPQSAPHGVVFLHVTYVPA
QEKNFTTAPAICHDGKAHFPREGVFVSNGTHWFVTQRNFYEPQIITTDNTFVSGNCDVVIGIV
NNTVYDPLQPELDSFKEELDKYFKNHTSPDVDLGDISGINASVVNIQKEIDRLNEVAKNLNES
LIDLQELGKYEQGSGYIPEAPRDGQAYVRKDGEWVLLSTFLGRSLEVLFQGPGHHHHHHHHS
AWSHPQFEKGGGSGGGGSGGSAWSHPQFEK (1288 amino acids, encoded by
SEQ ID NO: 2),

but also any mutants, splice variants, isoforms, orthologs, homologs, and variants of this sequence.

SARS-COV-2-S:
(SEQ ID NO: 5)
MFVFLVLLPLVSSQCVNLTTRTQLPPAYTNSFTRGVYYPDKVFRSSVLHSTQDLFLPFFSNVT
WFHAIHVSGTNGTKRFDNPVLPFNDGVYFASTEKSNIIRGWIFGTTLDSKTQSLLIVNNATNV
VIKVCEFQFCNDPFLGVYYHKNNKSWMESEFRVYSSANNCTFEYVSQPFLMDLEGKQGNFK
NLREFVFKNIDGYFKIYSKHTPINLVRDLPQGFSALEPLVDLPIGINITRFQTLLALHRSYLTPG
DSSSGWTAGAAAYYVGYLQPRTFLLKYNENGTITDAVDCALDPLSETKCTLKSFTVEKGIYQ
TSNFRVQPTESIVRFPNITNLCPFGEVENATRFASVYAWNRKRISNCVADYSVLYNSASFSTFK
CYGVSPTKLNDLCFTNVYADSFVIRGDEVRQIAPGQTGKIADYNYKLPDDFTGCVIAWNSNN
LDSKVGGNYNYLYRLFRKSNLKPFERDISTEIYQAGSTPCNGVEGENCYFPLQSYGFQPTNGV
GYQPYRVVVLSFELLHAPATVCGPKKSTNLVKNKCVNFNFNGLTGTGVLTESNKKFLPFQQF
GRDIADTTDAVRDPQTLEILDITPCSFGGVSVITPGTNTSNQVAVLYQDVNCTEVPVAIHADQ
LTPTWRVYSTGSNVFQTRAGCLIGAEHVNNSYECDIPIGAGICASYQTQTNSPRRARSVASQSI
IAYTMSLGAENSVAYSNNSIAIPTNFTISVTTEILPVSMTKTSVDCTMYICGDSTECSNLLLQY
GSFCTQLNRALTGIAVEQDKNTQEVFAQVKQIYKTPPIKDFGGFNFSQILPDPSKPSKRSFIEDL
LFNKVTLADAGFIKQYGDCLGDIAARDLICAQKFNGLTVLPPLLTDEMIAQYTSALLAGTITS
GWTFGAGAALQIPFAMQMAYRFNGIGVTQNVLYENQKLIANQFNSAIGKIQDSLSSTASALG
KLQDVVNQNAQALNTLVKQLSSNFGAISSVLNDILSRLDKVEAEVQIDRLITGRLQSLQTYVT
QQLIRAAEIRASANLAATKMSECVLGQSKRVDFCGKGYHLMSFPQSAPHGVVFLHVTYVPA
QEKNFTTAPAICHDGKAHFPREGVFVSNGTHWFVTQRNFYEPQIITTDNTFVSGNCDVVIGIV
NNTVYDPLQPELDSFKEELDKYFKNHTSPDVDLGDISGINASVVNIQKEIDRLNEVAKNLNES
LIDLQELGKYEQYIKWPWYIWLGFIAGLIAIVMVTIMLCCMTSCCSCLKGCCSCGSCCKFDED
DSEPVLKGVKLHYT, (1273 amino acids, encoded by SEQ ID NO: 6),

but also any mutants, splice variants, isoforms, orthologs, homologs, and variants of this sequence. In some embodiments, the CoV-S comprises a polypeptide sequence having at least 85%, 90%, 95%, 96%, 97%, 98%, or 99% identity to either SEQ ID NO:1 or SEQ ID NO:5.

“Effective treatment or prevention of CoV infection” herein refers to eliminating CoV from the subject or preventing the expansion of CoV in the subject or eliminating or reducing the symptoms such as fever, cough, shortness of breath, runny nose, congestion, conjunctivitis, and/or gastrointestinal symptoms after administration of an effective amount of an anti-CoV-S antibody or antigen-binding fragment thereof according to the disclosure. In some instances, effective treatment may eliminate the need for the subject to be placed on a ventilator or reduce the time the subject needs to be on a ventilator. The treatment may be effected as a monotherapy or in association with another active agent such as an antiviral agent or anti-inflammatory agent by way of example.

As used herein, “treatment” is an approach for obtaining beneficial or desired clinical results. For purposes of this disclosure, beneficial or desired clinical results include, but are not limited to, one or more of the following: improvement in any aspect of COV-S-related conditions such as fever or cough. For example, in the context of CoV infection treatment this includes lessening severity, alleviation of fever, cough, shortness of breath, and other associated symptoms, reducing frequency of recurrence, increasing the quality of life of those suffering from the CoV-related symptoms, and decreasing dose of other medications required to treat the CoV-related symptoms. Other associated symptoms include, but are not limited to, diarrhea, conjunctivitis, loss of smell, and loss of taste. Still other symptoms which may be alleviated or prevented include inflammation, cytokine storm and/or sepsis.

“Reducing incidence” or “prophylaxis” or “prevention” means any of reducing severity for a particular disease, condition, symptom, or disorder (the terms disease, condition, and disorder are used interchangeably throughout the application). Reduction in severity includes reducing drugs and/or therapies generally used for the condition by, for example, reducing the need for, amount of, and/or exposure to drugs or therapies. Reduction in severity also includes reducing the duration, and/or frequency of the particular condition, symptom, or disorder (including, for example, delaying or increasing time to next episodic attack in an individual). This further includes eliminating the need for the subject to be placed on a ventilator or reducing the time the subject needs to be on a ventilator.

“Ameliorating” one or more symptoms of CoV infection-related conditions means a lessening or improvement of one or more symptoms of the condition, e.g., fever or cough or shortness of breath as compared to not administering an anti-CoV-S antagonist antibody. “Ameliorating” also includes shortening or reduction in duration of a symptom. Again, this may include eliminating the need for the subject to be placed on a ventilator or reducing the time the subject needs to be on a ventilator.

As used herein, “controlling CoV-related symptom” or “controlling” another CoV-S-related condition refers to maintaining or reducing severity or duration of one or more symptoms of the condition (as compared to the level before treatment). For example, the duration or severity or frequency of symptoms is reduced by at least about any of 10%, 20%, 30%, 40%, 50%, 60%, 70%, 80%, 90%, or 100% in the individual as compared to the level before treatment. The reduction in the duration or severity, or frequency of symptoms can last for any length of time, e.g., 2 weeks, 4 weeks (1 month), 8 weeks (2 months), 16 weeks (3 months), 4 months, 5 months, 6 months, 9 months, 12 months, etc.

As used therein, “delaying” the development of a CoV-S-related condition such as shortness of breath, bronchitis, or pneumonia e.g., interstitial), means to defer, hinder, slow, retard, stabilize, and/or postpone progression of the condition or disease. This delay can be of varying lengths of time, depending on the history of the condition or disease and/or individuals being treated. As is evident to one skilled in the art, a sufficient or significant delay can, in effect, encompass prevention, in that the individual does not develop symptoms. A method that “delays” development of the symptom is a method that reduces probability of developing the symptom in a given time frame and/or reduces extent of the symptoms in a given time frame, when compared to not using the method. Such comparisons are typically based on clinical studies, using a statistically significant number of subjects.

“Development” or “progression” of a CoV-related condition such as cough or fever means initial manifestations and/or ensuing progression of the disorder. Development of cough or fever can be detectable and assessed using standard clinical techniques as well known in the art. However, development also refers to progression that may be undetectable. For purpose of this disclosure, development, or progression refers to the biological course of the symptoms. “Development” includes occurrence, recurrence, and onset. As used herein “onset” or “occurrence” of a condition includes initial onset and/or recurrence.

As used herein, an “effective dosage” or “effective amount” of drug, compound, or pharmaceutical composition is an amount sufficient to effect beneficial or desired results. For prophylactic use, beneficial or desired results include results such as eliminating or reducing the risk, lessening the severity, or delaying the outset of the disease, including biochemical, histological, and/or behavioral symptoms of the disease, its complications and intermediate pathological phenotypes presenting during development of the disease. For therapeutic use, beneficial or desired results include clinical results such as reducing symptom intensity, duration, or frequency, and decreasing one or more symptoms resulting from CoV infection, including its complications and intermediate pathological phenotypes presenting during development of the disease, increasing the quality of life of those suffering from the disease, decreasing the dose of other medications required to treat the disease, enhancing effect of another medication, and/or delaying the progression of the disease of patients, eliminating the need for the subject to be placed on a ventilator or reducing the time the subject needs to be on a ventilator.

An effective dosage can be administered in one or more administrations. For purposes of this disclosure, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

A “suitable host cell” or “host cell” generally includes any cell wherein the subject anti-CoV-S antibodies and antigen-binding fragments thereof can be produced recombinantly using techniques and materials readily available. For example, the anti-CoV-S antibodies and antigen-binding fragments thereof of the present disclosure can be produced in genetically engineered host cells according to conventional techniques. Suitable host cells are those cell types that can be transformed or transfected with exogenous DNA and grown in culture, and include bacteria, fungal cells (e.g., yeast), and cultured higher eukaryotic cells (including cultured cells of multicellular organisms), particularly cultured mammalian cells, e.g., human or non-human mammalian cells. In an exemplary embodiment these antibodies may be expressed in CHO cells. Techniques for manipulating cloned DNA molecules and introducing exogenous DNA into a variety of host cells are disclosed by Sambrook et al., Molecular Cloning: A Laboratory Manual, 2nd ed., Cold Spring Harbor, N.Y.: Cold Spring Harbor Laboratory Press (1989), and Current Protocols in Molecular Biology, Ausubel et al., editors, New York, NY: Green and Wiley and Sons (1993).

In some exemplary embodiments the antibodies may be expressed in mating competent yeast, e.g., any haploid, diploid or tetraploid yeast that can be grown in culture. Yeast useful in fermentation expression methods may exist in a haploid, diploid, or other polyploid form.

A “selectable marker” herein refers to a gene or gene fragment that confers a growth phenotype (physical growth characteristic) on a cell receiving that gene as, for example through a transformation event. The selectable marker allows that cell to survive and grow in a selective growth medium under conditions in which cells that do not receive that selectable marker gene cannot grow. Selectable marker genes generally fall into several types, including positive selectable marker genes such as a gene that confers on a cell resistance to an antibiotic or other drug, temperature when two temperature sensitive (“ts”) mutants are crossed or a ts mutant is transformed; negative selectable marker genes such as a biosynthetic gene that confers on a cell the ability to grow in a medium without a specific nutrient needed by all cells that do not have that biosynthetic gene, or a mutagenized biosynthetic gene that confers on a cell inability to grow by cells that do not have the wild type gene; and the like.

An “expression vector” herein refers to DNA vectors containing elements that facilitate manipulation for the expression of a foreign protein within the target host cell, e.g., a bacterial, insect, yeast, plant, amphibian, reptile, avian, or mammalian cell, e.g., a CHO or HEK cell. Conveniently, manipulation of sequences and production of DNA for transformation may first performed in a bacterial host, e.g. E. coli, and usually vectors will include sequences to facilitate such manipulations, including a bacterial origin of replication and appropriate bacterial selection marker. Selection markers encode proteins necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, (b) complement auxotrophic deficiencies, or (c) supply critical nutrients not available from complex media. Exemplary vectors and methods for transformation of yeast are described, for example, in Burke, D., Dawson, D., & Stearns, T., Methods in yeast genetics: a Cold Spring Harbor Laboratory course manual, Plainview, NY: Cold Spring Harbor Laboratory Press (2000). Expression vectors for use in the methods of the disclosure may include yeast or mammalian specific sequences, including a selectable auxotrophic or drug marker for identifying transformed host strains. A drug marker may further be used to amplify copy number of the vector in a yeast host cell.

The polypeptide coding sequence of interest is operably linked to transcriptional and translational regulatory sequences that provide for expression of the polypeptide in the desired host cells, e.g., yeast or mammalian cells. These vector components may include, but are not limited to, one or more of the following: an enhancer element, a promoter, and a transcription termination sequence. Sequences for the secretion of the polypeptide may also be included, e.g. a signal sequence, and the like. An origin of replication, e.g., a yeast or mammalian origin of replication, is optional, as expression vectors may be integrated into the host cell genome.

Nucleic acids are “operably linked” when placed into a functional relationship with another nucleic acid sequence. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites or alternatively via a PCR/recombination method familiar to those skilled in the art (GATEWAY® Technology (universal method for cloning DNA); Invitrogen, Carlsbad California). If such sites do not exist, the synthetic oligonucleotide adapters or linkers are used in accordance with conventional practice.

Promoters are untranslated sequences located upstream (5′) to the start codon of a structural gene (generally within about 100 to 1000 bp) that control the transcription and translation of particular nucleic acid sequences to which they are operably linked. Such promoters fall into several classes: inducible, constitutive, and repressible promoters (that increase levels of transcription in response to absence of a repressor). Inducible promoters may initiate increased levels of transcription from DNA under their control in response to some change in culture conditions, e.g., the presence or absence of a nutrient or a change in temperature.

The promoter fragment may also serve as the site for homologous recombination and integration of the expression vector into the same site in the host cell, e.g., yeast or mammalian cell, genome; alternatively, a selectable marker may be used as the site for homologous recombination. Suitable promoters for use in different eukaryotic and prokaryotic cells are well known and commercially available.

The polypeptides of interest may be produced recombinantly not only directly, but also as a fusion polypeptide with a heterologous polypeptide, e.g. a signal sequence or other polypeptide having a specific cleavage site at the N-terminus of the mature protein or polypeptide. In general, the signal sequence may be a component of the vector, or it may be a part of the polypeptide coding sequence that is inserted into the vector. The heterologous signal sequence selected preferably is one that is recognized and processed through one of the standard pathways available within the host cell, e.g., a mammalian cell, an insect cell, or a yeast cell. Additionally, these signal peptide sequences may be engineered to provide for enhanced secretion in expression systems. Secretion signals of interest also include mammalian and yeast signal sequences, which may be heterologous to the protein being secreted, or may be a native sequence for the protein being secreted. Signal sequences include pre-peptide sequences, and in some instances may include propeptide sequences. Many such signal sequences are known in the art, including the signal sequences found on immunoglobulin chains, e.g., K28 preprotoxin sequence, PHA-E, FACE, human MCP-1, human serum albumin signal sequences, human Ig heavy chain, human Ig light chain, and the like. For example, see Hashimoto et. al., Protein Eng., 11 (2): 75 (1998); and Kobayashi et. al., Therapeutic Apheresis, 2 (4): 257 (1998)).

Transcription may be increased by inserting a transcriptional activator sequence into the vector. These activators are cis-acting elements of DNA, usually about from 10 to 300 bp, which act on a promoter to increase its transcription. Transcriptional enhancers are relatively orientation and position independent, having been found 5′ and 3′ to the transcription unit, within an intron, as well as within the coding sequence itself. The enhancer may be spliced into the expression vector at a position 5′ or 3′ to the coding sequence, but is preferably located at a site 5′ from the promoter.

Expression vectors used in eukaryotic host cells may also contain sequences necessary for the termination of transcription and for stabilizing the mRNA. Such sequences are commonly available from 3′ to the translation termination codon, in untranslated regions of eukaryotic or viral DNAs or cDNAs. These regions contain nucleotide segments transcribed as polyadenylated fragments in the untranslated portion of the mRNA.

Construction of suitable vectors containing one or more of the above-listed components employs standard ligation techniques or PCR/recombination methods. Isolated plasmids or DNA fragments are cleaved, tailored, and re-ligated in the form desired to generate the plasmids required or via recombination methods. For analysis to confirm correct sequences in plasmids constructed, the ligation mixtures are used to transform host cells, and successful transformants selected by antibiotic resistance (e.g. ampicillin or Zeocin) where appropriate. Plasmids from the transformants are prepared, analyzed by restriction endonuclease digestion, and/or sequenced.

As an alternative to restriction and ligation of fragments, recombination methods based on specific attachment (“att”) sites and recombination enzymes may be used to insert DNA sequences into a vector. Such methods are described, for example, by Landy, Ann. Rev. Biochem., 58:913-949 (1989); and are known to those of skill in the art. Such methods utilize intermolecular DNA recombination that is mediated by a mixture of lambda and E. coli-encoded recombination proteins. Recombination occurs between att sites on the interacting DNA molecules. For a description of att sites see Weisberg and Landy, Site-Specific Recombination in Phage Lambda, in Lambda II, p. 211-250, Cold Spring Harbor, NY: Cold Spring Harbor Press (1983). The DNA segments flanking the recombination sites are switched, such that after recombination, the att sites are hybrid sequences comprised of sequences donated by each parental vector. The recombination can occur between DNAs of any topology.

Att sites may be introduced into a sequence of interest by ligating the sequence of interest into an appropriate vector; generating a PCR product containing att B sites through the use of specific primers; generating a cDNA library cloned into an appropriate vector containing att sites; and the like.

Folding, as used herein, refers to the three-dimensional structure of polypeptides and proteins, where interactions between amino acid residues act to stabilize the structure. While non-covalent interactions are important in determining structure, usually the proteins of interest will have intra- and/or intermolecular covalent disulfide bonds formed by two cysteine residues. For naturally occurring proteins and polypeptides or derivatives and variants thereof, the proper folding is typically the arrangement that results in optimal biological activity, and can conveniently be monitored by assays for activity, e.g. ligand binding, enzymatic activity, etc.

In some instances, for example where the desired product is of synthetic origin, assays based on biological activity will be less meaningful. The proper folding of such molecules may be determined on the basis of physical properties, energetic considerations, modeling studies, and the like.

The expression host may be further modified by the introduction of sequences encoding one or more enzymes that enhance folding and disulfide bond formation, i.e. foldases, chaperonins, etc. Such sequences may be constitutively or inducibly expressed in the host cell, using vectors, markers, etc. as known in the art. Preferably the sequences, including transcriptional regulatory elements sufficient for the desired pattern of expression, are stably integrated in the yeast genome through a targeted methodology.

For example, the eukaryotic protein disulfide isomerase (“PDI”) is not only an efficient catalyst of protein cysteine oxidation and disulfide bond isomerization, but also exhibits chaperone activity. Co-expression of PDI can facilitate the production of active proteins having multiple disulfide bonds. Also of interest is the expression of immunoglobulin heavy chain binding protein (“BIP”); cyclophilin; and the like.

Cultured mammalian cells are exemplary hosts for production of the disclosed anti-CoV-S antibodies and antigen-binding fragments thereof. As mentioned CHO cells are particularly suitable for expression of antibodies. Many procedures are known in the art for manufacturing monoclonal antibodies in mammalian cells. (See, Galfre, G. and Milstein, C., Methods Enzym., 73:3-46, 1981; Basalp et al., Turk. J. Biol., 24:189-196, 2000; Wurm, F. M., Nat. Biotechnol., 22:1393-1398, 2004; and Li et al., mAbs, 2 (5): 466-477, 2010). As mentioned in further detail infra, common host cell lines employed in mammalian monoclonal antibody manufacturing schemes include, but are not limited to, human embryonic retinoblast cell line PER.C6® (Crucell N.V., Leiden, The Netherlands), NS0 murine myeloma cells (Medical Research Council, London, UK), CV1 monkey kidney cell line, 293 human embryonic kidney cell line, BHK baby hamster kidney cell line, VERO African green monkey kidney cell line, human cervical carcinoma cell line HELA, MDCK canine kidney cells, BRL buffalo rat liver cells, W138 human lung cells, HepG2 human liver cells, MMT mouse mammary tumor cells, TRI cells, MRC5 cells, Fs4 cells, myeloma or lymphoma cells, or Chinese Hamster (Cricetulus griseus) Ovary (CHO) cells, and the like. Many different subclones or sub-cell lines of CHO cells known in the art that are useful and optimized for production of recombinant monoclonal antibodies, such as the DP12 (CHO K1 dhfr-) cell line, NS0 cells are a non-Ig secreting, non-light chain-synthesizing subclone of NS-1 cells that are resistant to azaguanine. Other Chinese Hamster and CHO cells are commercially available (from ATCC, etc.), including CHO-DXB11 (CHO-DUKX), CHO-pro3, CHO-DG44, CHO 1-15, CHO DP-12, Lec2, M1WT3, Lec8, pgsA-745, and the like, all of which are genetically altered to optimize the cell line for various parameters. Monoclonal antibodies are commonly manufactured using a batch fed method whereby the monoclonal antibody chains are expressed in a mammalian cell line and secreted into the tissue culture medium in a bioreactor. Medium (or feed) is continuously supplied to the bioreactor to maximize recombinant protein expression. Recombinant monoclonal antibody is then purified from the collected media. In some circumstances, additional steps are needed to reassemble the antibodies through reduction of disulfide bonds, etc. Such production methods can be scaled to be as large as 10,000 L in a single batch or more. It is now routine to obtain as much as 20 pg/cell/day through the use of such cell lines and methodologies, providing titers as high as 10 g/L or more, amounting to 15 to 100 kg from bioreactors of 10 kL to 25 kL. (Li et al., 2010). Various details of this production methodology, including cloning of the polynucleotides encoding the antibodies into expression vectors, transfecting cells with these expression vectors, selecting for transfected cells, and expressing and purifying the recombinant monoclonal antibodies from these cells are provided below.

For recombinant production of an anti-CoV-S antibody or antigen-binding fragment in mammalian cells, nucleic acids encoding the antibody or fragment thereof are generally inserted into a replicable vector for further cloning (amplification of the DNA) or for expression. DNA encoding the antibody is readily isolated or synthesized using conventional procedures (e.g., by using oligonucleotide probes that are capable of binding specifically to DNAs encoding the heavy and light chains of the antibody). The vector components generally include, but are not limited to, one or more of the following: a signal sequence, an origin of replication, one or more marker genes, an enhancer element, a promoter, and a transcription termination sequence. Selection of promoters, terminators, selectable markers, vectors, and other elements is a matter of routine design within the level of ordinary skill in the art. Many such elements are known in the art and are available through commercial suppliers.

The antibodies of this disclosure may be produced recombinantly not only directly, but also as a fusion polypeptide with a heterologous polypeptide, which is preferably a signal sequence or other polypeptide having a specific cleavage site at the N-terminus of the mature protein or polypeptide. The homologous or heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available.

Such expression vectors and cloning vectors will generally contain a nucleic acid sequence that enables the vector to replicate in one or more selected host cells. Typically, in cloning vectors this sequence is one that enables the vector to replicate independently of the host chromosomal DNA, and includes origins of replication or autonomously replicating sequences. Such sequences are well known for a variety of bacteria, yeast, and viruses, e.g., the origin of replication from the plasmid pBR322 is suitable for most Gram-negative bacteria, the 2mu plasmid origin is suitable for yeast, and various viral origins (Simian Virus 40 (“SV40”), polyoma, adenovirus, vesicular stomatitis virus (“VSV”), or bovine papillomavirus (“BPV”) are useful for cloning vectors in mammalian cells. Generally, the origin of replication component is not needed for mammalian expression vectors (the SV40 origin may typically be used only because it contains the early promoter).

These vectors will also typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” Examples of such dominant selection use the drugs neomycin, mycophenolic acid, and hygromycin. An exemplary selectable marker is a gene encoding resistance to the antibiotic neomycin. Selection is carried out in the presence of a neomycin-type drug, such as G-418 or the like. Those cells that are successfully transformed with a heterologous gene produce a protein conferring drug resistance and thus survive the selection regimen.

Selection systems can also be used to increase the expression level of the gene of interest, a process referred to as “amplification.” Amplification of transfectants typically occurs by culturing the cells in the presence of a low level of the selective agent and then increasing the amount of selective agent to select for cells that produce high levels of the products of the introduced genes. Exemplary suitable selectable markers for mammalian cells are those that enable the identification of cells competent to take up the antibody nucleic acid, such as dihydrofolate reductase (“DHFR”), thymidine kinase, metallothionein-I and -II, preferably primate metallothionein genes, adenosine deaminase, ornithine decarboxylase, etc.

For example, an amplifiable selectable marker for mammalian cells is dihydrofolate reductase, which confers resistance to methotrexate. Other drug resistance genes (e.g. hygromycin resistance, multi-drug resistance, puromycin acetyltransferase) can also be used. Cells transformed with the DHFR selection gene are first identified by culturing all of the transformants in a culture medium that contains methotrexate (“MTX”), a competitive antagonist of DHFR. An appropriate host cell when wild-type DHFR is employed is the Chinese hamster ovary (“CHO”) cell line deficient in DHFR activity.

Alternatively, host cells (particularly wild-type hosts that contain endogenous DHFR) transformed or co-transformed with DNA sequences encoding antibody, wild-type DHFR protein, and another selectable marker such as aminoglycoside 3′-phosphotransferase (“APH”) can be selected by cell growth in medium containing a selection agent for the selectable marker such as an aminoglycosidic antibiotic, e.g., kanamycin, neomycin, or G-418. See U.S. Pat. No. 4,965,199.

These vectors may comprise an enhancer sequence that facilitates transcription of a DNA encoding the antibody. Many enhancer sequences are known from mammalian genes (for example, globin, elastase, albumin, alpha-fetoprotein, and insulin). A frequently used enhancer is one derived from a eukaryotic cell virus. Examples thereof include the SV40 enhancer on the late side of the replication origin (bp 100-270), the cytomegalovirus early promoter enhancer, the polyoma enhancer on the late side of the replication origin, and adenovirus enhancers (See also Yaniv, Nature, 297:17-18 (1982) on enhancing elements for activation of eukaryotic promoters). The enhancer may be spliced into the vector at a position 5′ or 3′ to the antibody-encoding sequence, but is preferably located at a site 5′ from the promoter.

Expression and cloning vectors will also generally comprise a promoter that is recognized by the host organism and is operably linked to the antibody nucleic acid. Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Antibody transcription from vectors in mammalian host cells may be controlled, for example, by promoters obtained from the genomes of viruses such as polyoma virus, fowlpox virus, adenovirus (such as Adenovirus 2), BPV, avian sarcoma virus, cytomegalovirus, a retrovirus, hepatitis-B virus, and most preferably SV40, from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the BPV as a vector is disclosed in U.S. Pat. No. 4,419,446. A modification of this system is described in U.S. Pat. No. 4,601,978. See also Reyes et al., Nature, 297:598-601 (1982) on expression of human beta-interferon cDNA in mouse cells under the control of a thymidine kinase promoter from herpes simplex virus. Alternatively, the Rous sarcoma virus long terminal repeat can be used as the promoter.

Strong transcription promoters can be used, such as promoters from SV40, cytomegalovirus, or myeloproliferative sarcoma virus. See, e.g., U.S. Pat. No. 4,956,288 and U.S. Patent Publication No. 20030103986. Other suitable promoters include those from metallothionein genes (U.S. Pat. Nos. 4,579,821 and 4,601,978) and the adenovirus major late promoter. Expression vectors for use in mammalian cells include pZP-1, pZP-9, and pZMP21, which have been deposited with the American Type Culture Collection, 10801 University Blvd., Manassas, VA. USA under accession numbers 98669, 98668, and PTA-5266, respectively, and derivatives of these vectors.

Expression vectors used in eukaryotic host cells (yeast, fungus, insect, plant, animal, human, or a nucleated cell from other multicellular organism) will also generally contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the antibody. One useful transcription termination component is the bovine growth hormone polyadenylation region. See WO 94/11026 and the expression vector disclosed therein.

Suitable host cells for cloning or expressing the subject antibodies include prokaryote, yeast, or higher eukaryote cells described above. However, interest has been greatest in vertebrate cells, and propagation of vertebrate cells in culture has become a routine procedure. Examples of useful mammalian host cell lines are monkey kidney CV1 line transformed by SV40 (COS-1 (ATCC No. CRL 1650); and COS-7, ATCC CRL 1651); human embryonic kidney line (293 or 293 cells subcloned for growth in suspension culture, (ATCC No. CRL 1573; Graham et al., J. Gen. Virol., 36:59-72 (1977)); baby hamster kidney cells (BHK, ATCC CCL 10, ATCC No. CRL 1632; BHK 570, ATCC No. CRL 10314); CHO cells (CHO-K1, ATCC No. CCL 61; CHO-DG44, Urlaub et al., Proc. Natl. Acad. Sci. USA, 77:4216-4220 (1980)); mouse sertoli cells (TM4, Mather, Biol. Reprod., 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 (1982)); MRC 5 cells; FS4 cells; and a human hepatoma line (Hep G2). Additional suitable cell lines are known in the art and available from public depositories such as the American Type Culture Collection, Manassas, VA.

Host cells are transformed with the above-described expression or cloning vectors for antibody production and cultured in conventional nutrient media modified as appropriate for inducing promoters, selecting transformants, or amplifying the genes encoding the desired sequences as discussed supra.

The mammalian host cells used to produce the antibody of this disclosure may be cultured in a variety of media. Commercially available media such as Ham's F10 (Sigma-Aldrich Corporation, St. Louis, MO), Minimal Essential Medium ((“MEM” (Sigma-Aldrich Corporation, St. Louis, MO), Roswell Park Memorial Institute-1640 medium (“RPMI-1640”, Sigma-Aldrich Corporation, St. Louis, MO), and Dulbecco's Modified Eagle's Medium ((“DMEM” Sigma-Aldrich Corporation, St. Louis, MO) are suitable for culturing the host cells. In addition, any of the media described in Ham et al., Meth. Enz., 58:44 (1979), Barnes et al., Anal. Biochem., 102:255 (1980), U.S. Pat. Nos. 4,767,704; 4,657,866; 4,927,762; 4,560,655; or 5,122,469; WO 90/03430; WO 87/00195; or Reexam U.S. Pat. No. 30,985 can be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan. Methods of development and optimization of media and culture conditions are known in the art (See, Gronemeyer et al., Bioengineering, 1 (4): 188-212, 2014).

After culture conditions are optimized and a preferred cell line clone is selected, these cells are cultured (either adherent cells or suspension cultures) most typically in a batch-fed process in a bioreactor (many models are commercially available) that involves continuously feeding the cell culture with medium and feed, optimized for the particular cell line chosen and selected for this purpose. (See, Butler, M., Appl. Microbiol. Biotechnol., 68:283-291, 2005; and Kelley, B., mAb, 1 (5): 443-452, 2009). Perfusion systems are also available in which media and feed are continuously supplied to the culture while the same volume of media is being withdrawn from the bioreactor. (Wurm, 2004). Synthetic media, also commercially available, are available for growing cells in a batch-fed culture, avoiding the possibility of contamination from outside sources, such as with the use of animal components, such as bovine serum albumin, etc. However, animal-component-free hydrolysates are commercially available to help boost cell density, culture viability and productivity. (Li et al., 2010). Many studies have been performed in an effort to optimize cell culture media, including careful attention to head space available in roller bottles, redox potentials during growth and expression phases, presence of reducing agents to maintain disulfide bonds during production, etc. (See, for instance, Hutterer et al., mAbs, 5 (4): 608-613, 2013; and Mullan et al., BMC Proceed., 5 (Suppl 8): P110, 2011). Various methodologies have been developed to address the possibility of harmful oxidation during recombinant monoclonal antibody production. (See, for example, U.S. Pat. No. 8,574,869). Cultured cells may be grown by feeding nutrients continuously or as separately administered amounts. Often various process parameters such as cell concentration, pH, temperature, CO₂, dO₂, osmolality, amount of metabolites such as glucose, lactate, glutamine and glutamate, and the like, are monitored by the use of probes during the cell growth either on-line by direct connection to calibrated analyzers or off-line by intervention of operators. The culturing step also typically involves ensuring that the cells growing in culture maintain the transfected recombinant genes by any means known in the art for cell selection.

Following fermentation, i.e., upon reaching maximum cell growth and recombinant protein expression, the culturing step is typically followed by a harvesting step, whereby the cells are separated from the medium and a harvested cell culture media is thereby obtained. (See, Liu et al., mAbs, 2 (5): 480-499, 2010). Typically, various purification steps, involving column chromatography and the like, follow culturing to separate the recombinant monoclonal antibody from cell components and cell culture media components. The exact purification steps needed for this phase of the production of recombinant monoclonal antibodies depends on the site of expression of the proteins, i.e., in the cytosol of the cells themselves, or the more commonly preferred route of protein excreted into the cell culture medium. Various cell components may be separated using techniques known in the art such as differential centrifugation techniques, gravity-based cell settling, and/or size exclusion chromatograph/filtration techniques that can include tangential flow micro-filtration or depth filtration. (See, Pollock et al., Biotechnol. Bioeng., 110:206-219, 2013, and Liu et al., 2010). Centrifugation of cell components may be achieved on a large scale by use of continuous disk stack centrifuges followed by clarification using depth and membrane filters. (See, Kelley, 2009). Most often, after clarification, the recombinant protein is further purified by Protein A chromatography due to the high affinity of Protein A for the Fc domain of antibodies, and typically occurs using a low pH/acidification elution step (typically the acidification step is combined with a precautionary virus inactivation step). Flocculation and/or precipitation steps using acidic or cationic polyelectrolytes may also be employed to separate animal cells in suspension cultures from soluble proteins. (Liu et al., 2010). Lastly, anion- and cation-exchange chromatography, hydrophobic interaction chromatograph (“HIC”), hydrophobic charge induction chromatograph (HCIC), hydroxyapatite chromatography using ceramic hydroxyapatite (Ca₅(PO₄)₃OH)₂, and combinations of these techniques are typically used to polish the solution of recombinant monoclonal antibody. Final formulation and concentration of the desired monoclonal antibody may be achieved by use of ultracentrifugation techniques. Purification yields are typically 70 to 80%. (Kelley, 2009).

The terms “desired protein” or “desired antibody” herein are used interchangeably and refer generally to a parent antibody specific to a target, i.e., CoV-S or a chimeric or humanized antibody or a binding portion thereof derived therefrom as described herein. The term “antibody” is intended to include any polypeptide chain-containing molecular structure with a specific shape that fits to and recognizes an epitope, where one or more non-covalent binding interactions stabilize the complex between the molecular structure and the epitope. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammals, chicken, other avians, etc., are considered to be “antibodies.” Examples thereof include chimeric antibodies, human antibodies and other non-human mammalian antibodies, humanized antibodies, single chain antibodies (such as scFvs), camelbodies, nanobodies, IgNAR (single-chain antibodies which may be derived from sharks, for example), small-modular immunopharmaceuticals (“SMIPs”), and antibody fragments such as Fabs, Fab′, F(ab′)₂, and the like (See Streltsov et al., Protein Sci., 14 (11): 2901-9 (2005); Greenberg et al., Nature, 374 (6518): 168-73 (1995); Nuttall et al., Mol. Immunol., 38 (4): 313-26 (2001); Hamers-Casterman et al., Nature, 363 (6428): 446-8 (1993); Gill et al., Curr. Opin. Biotechnol., (6): 653-8 (2006)).

For example, antibodies or antigen-binding fragments thereof may be produced by genetic engineering. In this technique, as with other methods, antibody-producing cells are sensitized to the desired antigen or immunogen. The messenger RNA isolated from antibody producing cells is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library is constructed by combining the heavy chain gene library with the light chain gene library. This results in a library of clones that co-express a heavy and light chain (resembling the Fab fragment or antigen-binding fragment of an antibody molecule). The vectors that carry these genes are co-transfected into a host cell. When antibody gene synthesis is induced in the transfected host, the heavy and light chain proteins self-assemble to produce active antibodies that can be detected by screening with the antigen or immunogen.

Antibody coding sequences of interest include those encoded by native sequences, as well as nucleic acids that, by virtue of the degeneracy of the genetic code, are not identical in sequence to the disclosed nucleic acids, and variants thereof. Variant polypeptides can include amino acid (“aa”) substitutions, additions, or deletions. The amino acid substitutions can be conservative amino acid substitutions or substitutions to eliminate non-essential amino acids, such as to alter a glycosylation site, or to minimize misfolding by substitution or deletion of one or more cysteine residues that are not necessary for function. Variants can be designed so as to retain or have enhanced biological activity of a particular region of the protein (e.g., a functional domain, catalytic amino acid residues, etc). Variants also include fragments of the polypeptides disclosed herein, particularly biologically active fragments and/or fragments corresponding to functional domains. Techniques for in vitro mutagenesis of cloned genes are known. Also included in the subject disclosure are polypeptides that have been modified using ordinary molecular biological techniques so as to improve their resistance to proteolytic degradation or to optimize solubility properties or to render them more suitable as a therapeutic agent.

Chimeric antibodies may be made by recombinant means by combining the VL and VH regions, obtained from antibody producing cells of one species with the constant light and heavy chain regions from another. Typically, chimeric antibodies utilize rodent or rabbit variable regions and human constant regions, in order to produce an antibody with predominantly human domains. The production of such chimeric antibodies is well known in the art, and may be achieved by standard means (as described, e.g., in U.S. Pat. No. 5,624,659, incorporated herein by reference in its entirety). It is further contemplated that the human constant regions of chimeric antibodies of the disclosure may be selected from IgG1, IgG2, IgG3, and IgG4 constant regions.

Humanized antibodies are engineered to contain even more human-like immunoglobulin domains, and incorporate only the complementarity determining regions of the animal-derived antibody. This is accomplished by carefully examining the sequence of the hyper-variable loops of the variable regions of the monoclonal antibody and fitting them to the structure of the human antibody chains. Although facially complex, the process is straightforward in practice. See, e.g., U.S. Pat. No. 6,187,287, incorporated fully herein by reference.

In addition to entire immunoglobulins (or their recombinant counterparts), immunoglobulin fragments comprising the epitope binding site (e.g., Fab′, F(ab′)₂, or other fragments) may be synthesized. “Fragment” or minimal immunoglobulins may be designed utilizing recombinant immunoglobulin techniques. For instance, “Fv” immunoglobulins for use in the present disclosure may be produced by synthesizing a fused variable light chain region and a variable heavy chain region. Combinations of antibodies are also of interest, e.g. diabodies, which comprise two distinct Fv specificities. In another embodiment, small molecule immunopharmaceuticals (“SMIPs”), camelbodies, nanobodies, and IgNAR are encompassed by immunoglobulin fragments.

Immunoglobulins and fragments thereof may be modified post-translationally, e.g. to add effector moieties such as chemical linkers, detectable moieties, such as fluorescent dyes, enzymes, toxins, substrates, bioluminescent materials, radioactive materials, chemiluminescent moieties, and the like, or specific binding moieties, such as streptavidin, avidin, or biotin, and the like may be utilized in the methods and compositions of the present disclosure. Examples of additional effector molecules are provided infra.

A polynucleotide sequence “corresponds” to a polypeptide sequence if translation of the polynucleotide sequence in accordance with the genetic code yields the polypeptide sequence (i.e., the polynucleotide sequence “encodes” the polypeptide sequence), one polynucleotide sequence “corresponds” to another polynucleotide sequence if the two sequences encode the same polypeptide sequence.

A “heterologous” region or domain of a DNA construct is an identifiable segment of DNA within a larger DNA molecule that is not found in association with the larger molecule in nature. Thus, when the heterologous region encodes a mammalian gene, the DNA flanking the gene usually does not flank the mammalian genomic DNA in the genome of the source organism. Another example of a heterologous region is a construct where the coding sequence itself is not found in nature (e.g., a cDNA where the genomic coding sequence contains introns or synthetic sequences having codons different than the native gene). Allelic variations or naturally-occurring mutational events do not give rise to a heterologous region of DNA as defined herein.

A “coding sequence” is an in-frame sequence of codons that correspond to or encode a protein or peptide sequence. Two coding sequences correspond to each other if the sequences or their complementary sequences encode the same amino acid sequences. A coding sequence in association with appropriate regulatory sequences may be transcribed and translated into a polypeptide. A polyadenylation signal and transcription termination sequence will usually be located 3′ to the coding sequence. A “promoter sequence” is a DNA regulatory region capable of initiating transcription of a downstream (3′ direction) coding sequence, and typically contain additional sites for binding of regulatory molecules, e.g., transcription factors, that affect the transcription of the coding sequence. A coding sequence is “under the control” of the promoter sequence or “operatively linked” to the promoter when RNA polymerase binds the promoter sequence in a cell and transcribes the coding sequence into mRNA, which is then in turn translated into the protein encoded by the coding sequence.

The general structure of antibodies in vertebrates now is well understood. See Edelman, G. M., Ann. N.Y. Acad. Sci., 190:5 (1971). Antibodies consist of two identical light polypeptide chains of molecular weight approximately 23,000 daltons (the “light chain”), and two identical heavy chains of molecular weight 53,000-70,000 (the “heavy chain”). The four chains are joined by disulfide bonds in a “Y” configuration wherein the light chains bracket the heavy chains starting at the mouth of the “Y” configuration. The “branch” portion of the “Y” configuration is designated the Fab region; the stem portion of the “Y” configuration is designated the Fc region. The amino acid sequence orientation runs from the N-terminal end at the top of the “Y” configuration to the C-terminal end at the bottom of each chain. The N-terminal end possesses the variable region having specificity for the antigen that elicited it, and is approximately 100 amino acids in length, there being slight variations between light and heavy chain and from antibody to antibody.

The variable region is linked in each chain to a constant region that extends the remaining length of the chain and that within a particular class of antibody does not vary with the specificity of the antibody (i.e., the antigen eliciting it). There are five known major classes of constant regions that determine the class of the immunoglobulin molecule (IgG, IgM, IgA, IgD, and IgE corresponding to γ, μ, α, δ, and ε (gamma, mu, alpha, delta, or epsilon) heavy chain constant regions). The constant region or class determines subsequent effector function of the antibody, including activation of complement (see Kabat, E. A., Structural Concepts in Immunology and Immunochemistry, 2nd Ed., p. 413-436, New York, NY: Holt, Rinehart, Winston (1976)), and other cellular responses (see Andrews et al., Clinical Immunology, pp. 1-18, W. B. Sanders, Philadelphia, PA (1980); Kohl et al., Immunology, 48:187 (1983)); while the variable region determines the antigen with which it will react. Light chains are classified as either κ (kappa) or λ (lambda). Each heavy chain class can be prepared with either kappa or lambda light chain. The light and heavy chains are covalently bonded to each other, and the “tail” portions of the two heavy chains are bonded to each other by covalent disulfide linkages when the immunoglobulins are generated either by hybridomas or by B-cells.

The expression “variable region” or “VR” refers to the domains within each pair of light and heavy chains in an antibody that are involved directly in binding the antibody to the antigen. Each heavy chain has at one end a variable region (VH) followed by a number of constant domains. Each light chain has a variable region (VL) at one end and a constant domain at its other end; the constant domain of the light chain is aligned with the first constant domain of the heavy chain, and the light chain variable domain is aligned with the variable domain of the heavy chain.

The expressions “complementarity-determining region,” “hypervariable region,” or “CDR” refer to one or more of the hyper-variable or complementarity-determining regions (“CDRs”) found in the variable regions of light or heavy chains of an antibody (See Kabat et al., Sequences of Proteins of Immunological Interest, 4^{th} ed., Bethesda, MD: U.S. Dept. of Health and Human Services, Public Health Service, National Institutes of Health (1987)). These expressions include the hypervariable regions as defined by Kabat et al., (Sequences of Proteins of Immunological Interest, NIH Publication No. 91-3242, Bethesda, MD: U.S. Dept. of Health and Human Services, National Institutes of Health (1983)) or the hypervariable loops in 3-dimensional structures of antibodies (Chothia and Lesk, J. Mol. Biol., 196:901-917 (1987)). The CDRs in each chain are held in close proximity by framework regions (“FRs”) and, with the CDRs from the other chain, contribute to the formation of the antigen binding site. Within the CDRs there are select amino acids that have been described as the selectivity determining regions (“SDRs”) that represent the critical contact residues used by the CDR in the antibody-antigen interaction (see Kashmiri et al., Methods, 36 (1): 25-34 (2005)).

An “isolated antibody”, as used herein, is intended to refer to an antibody that is substantially free of other antibodies having different antigenic specificities (e.g., an isolated antibody that specifically binds CoV-S is substantially free of antibodies that specifically bind antigens other than CoV-S). An isolated antibody that specifically binds CoV-S may, however, have cross-reactivity to other antigens, such as CoV-S molecules from other species. Moreover, an isolated antibody may be substantially free of other cellular material and/or chemicals.

The phrase “specifically binds to CoV-S” as used herein, refers to the ability of an anti-CoV-S antibody or antigen-binding fragment thereof to interact with CoV-S with a dissociated constant (KD) of, for example, about 1,000 nM or less, about 500 nM or less, about 200 nM or less, about 100 nM or less, about 75 nM or less, about 25 nM or less, about 10 nM or less, about 1 nM or less, about 100 pM nM or less, about 10 pM nM or less, about 1 pM or less, or about 0.1 pM or less. In another embodiment, the phrase “specifically binds to CoV-S”, as used herein, refers to the ability of an anti-CoV-S antibody or antigen-binding fragment thereof to interact with CoV-S with a dissociation constant (KD) of between about 0.1 pM to 1,000 nM, between about 1 pM to 500 nM, between about 10 pM to 100 nM, between about 0.1 nM to 50 nM, or between about 1 nM to 50 nM. In one embodiment, KD is determined by surface plasmon resonance, ELISAs, radioimmunoassays, bio-layer interferometry (BLI), or by any other methods known in the art.

An “epitope” or “binding site” is an area or region on an antigen to which an antigen-binding peptide (such as an antibody) specifically binds. A protein epitope may comprise amino acid residues directly involved in the binding (also called immunodominant component of the epitope) and other amino acid residues, which are not directly involved in the binding, such as amino acid residues that are effectively blocked by the specifically antigen binding peptide (in other words, the amino acid residue is within the “footprint” of the specifically antigen binding peptide). The term epitope herein includes both types of amino acid binding sites in any particular region of CoV-S, e.g., SARS-COV-S or SARS-COV-2-S, that specifically binds to an anti-CoV-S antibody. In some embodiments, the epitope is a conserved site within the spike protein, e.g., SARS-COV-S or SARS-COV-2-S, e.g., the CR3022 site and the N343 proteoglycan site in the receptor binding domain (RBD), or the S2 domain. CoV-S may comprise a number of different epitopes, which may include, without limitation, (1) linear peptide antigenic determinants, (2) conformational antigenic determinants that consist of one or more non-contiguous amino acids located near each other in a mature CoV-S conformation; and (3) post-translational antigenic determinants that consist, either in whole or part, of molecular structures covalently attached to a CoV-S protein such as carbohydrate groups. In particular, the term “epitope” includes the specific residues in a protein or peptide, e.g., CoV-S, which are involved in the binding of an antibody to such protein or peptide as determined by known and accepted methods such as alanine scanning techniques or the use of various S protein portions with varying lengths.

The phrase that an antibody (e.g., first antibody) binds “substantially” or “at least partially” the same epitope as another antibody (e.g., second antibody) means that the epitope binding site for the first antibody comprises at least 10%, 20%, 30%, 40%, 50%, 60%, 70%, 80%, 90%, or more of the amino acid residues on the antigen that constitutes the epitope binding site of the second antibody. Also, that a first antibody binds substantially or partially the same or overlapping epitope as a second antibody means that the first and second antibodies compete in binding to the antigen, as described above. Thus, the term “binds to substantially the same epitope or determinant as” a monoclonal antibody means that an antibody “competes” with the antibody.

The phrase “binds to the same or overlapping epitope or determinant as” an antibody of interest means that an antibody “competes” with said antibody of interest for at least one, (e.g., at least 2, at least 3, at least 4, at least 5) or all residues on CoV-S to which said antibody of interest specifically binds. The identification of one or more antibodies that bind(s) to substantially or essentially the same epitope as the monoclonal antibodies described herein can be readily determined using alanine scanning. Additionally, any one of variety of immunological screening assays in which antibody competition can be assessed. A number of such assays are routinely practiced and well known in the art (see, e.g., U.S. Pat. No. 5,660,827, issued Aug. 26, 1997, which is specifically incorporated herein by reference). It will be understood that actually determining the epitope to which an antibody described herein binds is not in any way required to identify an antibody that binds to the same or substantially the same or overlapping epitope as the monoclonal antibody described herein.

For example, where the test antibodies to be examined are obtained from different source animals, or are even of a different Ig isotype, a simple competition assay may be employed in which the control antibody is mixed with the test antibody and then applied to a sample containing CoV-S. Protocols based upon ELISAs, radioimmunoassays, Western blotting, and the use of BIACORE® (GE Healthcare Life Sciences, Marlborough, MA) analysis are suitable for use in such simple competition studies.

In certain embodiments, the control anti-CoV-S antibody is pre-mixed with varying amounts of the test antibody (e.g., in ratios of about 1:1, 1:2, 1:10, or about 1:100) for a period of time prior to applying to the CoV-S (e.g., SARS-COV-S or SARS-COV-2-S) antigen sample. In other embodiments, the control and varying amounts of test antibody can simply be added separately and admixed during exposure to the SARS-COV-S or SARS-COV-2-S antigen sample. As long as bound antibodies can be distinguished from free antibodies (e.g., by using separation or washing techniques to eliminate unbound antibodies) and control antibody from the test antibody (e.g., by using species specific or isotype specific secondary antibodies or by specifically labeling the control antibody with a detectable label) it can be determined if the test antibody reduces the binding of the control antibody to the SARS-CoV-S or SARS-COV-2-S antigens, indicating that the test antibody recognizes substantially the same epitope as the control anti-CoV-S antibody. The binding of the (labeled) control antibody in the presence of a completely irrelevant antibody (that does not bind CoV-S) can serve as the control high value. The control low value can be obtained by incubating the labeled control antibody with the same but unlabeled control antibody, where competition would occur and reduce binding of the labeled antibody. In a test assay, a significant reduction in labeled antibody reactivity in the presence of a test antibody is indicative of a test antibody that recognizes substantially the same epitope, i.e., one that competes with the labeled control antibody. For example, any test antibody that reduces the binding of the control antibody to SARS-COV-S or SARS-COV-2-S by at least about 50%, such as at least about 60%, or more preferably at least about 70% (e.g., about 65-100%), at any ratio of test antibody between about 1:1 or 1:10 and about 1:100 is considered to be an antibody that binds to substantially the same or overlapping epitope or determinant as the control antibody.

Preferably, such test antibody will reduce the binding of the control antibody to SARS-COV-S or SARS-COV-2-S (or another CoV-S) antigen preferably at least about 50%, at least about 60%, at least about 80%, or at least about 90% (e.g., about 95%) of the binding of the control antibody observed in the absence of the test antibody.

A simple competition assay in which a test antibody is applied at saturating concentration to a surface onto which SARS-COV-S or SARS-COV-2-S (or another CoV-S) is immobilized also may be advantageously employed. The surface in the simple competition assay is preferably a BIACORE® (GE Healthcare Life Sciences, Marlborough, MA) chip (or other media suitable for surface plasmon resonance (“SPR”) analysis). The binding of a control antibody that binds SARS-COV-S or SARS-CoV-2-S to the COV-S-coated surface is measured. This binding to the SARS-COV-S- or SARS-COV-2-S-containing surface of the control antibody alone is compared with the binding of the control antibody in the presence of a test antibody. A significant reduction in binding to the SARS-COV-S- or SARS-COV-2-S-containing surface by the control antibody in the presence of a test antibody indicates that the test antibody recognizes substantially the same epitope as the control antibody such that the test antibody “competes” with the control antibody. Any test antibody that reduces the binding of control antibody by at least about 20% or more, at least about 40%, at least about 50%, at least about 70%, or more, can be considered to be an antibody that binds to substantially the same epitope or determinant as the control antibody. Preferably, such test antibody will reduce the binding of the control antibody to SARS-COV-S or SARS-COV-2-S by at least about 50% (e.g., at least about 60%, at least about 70%, or more). It will be appreciated that the order of control and test antibodies can be reversed; i.e. the control antibody can be first bound to the surface and then the test antibody is brought into contact with the surface thereafter in a competition assay. Preferably, the “sandwich-style” binding assay infra is used. Alternatively, the antibody having greater affinity for SARS-COV-S or SARS-COV-2-S antigen is bound to the SARS-COV-S- or SARS-COV-2-S-containing surface first, as it will be expected that the decrease in binding seen for the second antibody (assuming the antibodies are competing) will be of greater magnitude. Further examples of such assays are provided in e.g., Saunal and Regenmortel, J. Immunol. Methods, 183:33-41 (1995), the disclosure of which is incorporated herein by reference.

In addition, whether an antibody binds the same or overlapping epitope(s) on COV-S as another antibody or the epitope bound by a test antibody may in particular be determined using a Western-blot based assay. In this assay a library of peptides corresponding to the antigen bound by the antibody, the CoV-S protein, is made, that comprise overlapping portions of the protein, typically 10-25, 10-20, or 10-15 amino acids long. These different overlapping amino acid peptides encompassing the CoV-S sequence are synthesized and covalently bound to a PEPSPOTS™ nitrocellulose membrane (JPT Peptide Technologies, Berlin, Germany). Blots are then prepared and probed according to the manufacturer's recommendations.

Essentially, the immunoblot assay then detects by fluorometric means what peptides in the library bind to the test antibody and thereby can identify what residues on the antigen, i.e., COV-S, interact with the test antibody. (See U.S. Pat. No. 7,935,340, incorporated by reference herein).

Various epitope mapping techniques are known in the art. By way of example, X-ray co-crystallography of the antigen and antibody; NMR; SPR (e.g., at 25° or 37° C.); array-based oligo-peptide scanning (or “pepscan analysis”); site-directed mutagenesis (e.g., alanine scanning); mutagenesis mapping; hydrogen-deuterium exchange; phage display; and limited proteolysis are all epitope mapping techniques that are well known in the art (See, e.g., Epitope Mapping Protocols: Second Edition, Methods in Molecular Biology., editors Mike Schutkowski and Ulrich Reineke, 2^{nd} Ed., New York, NY: Humana Press (2009), and Epitope Mapping Protocols, Methods in Molecular Biology, editor Glenn Morris, 1^{st} Ed., New York, NY: Humana Press (1996), both of which are herein incorporated by referenced in their entirety).

The identification of one or more antibodies that bind(s) to substantially or essentially the same epitope as the monoclonal antibodies described herein, e.g., antibodies described in Tables 3-6, and e.g., VYD225, VYD224 (also known as ADI-80707), ADI-75696, VYD223 (also known as ADI-75865), ADI-75864, ADI-75620, ADI-75738, ADI-75700, ADI-75859, ADI-75684, ADI-75754, ADI-75648, ADI-75632, ADI-75741, ADI-75725, ADI-75717, ADI-75706, ADI-75699, ADI-75747, or ADI-75773, can be readily determined using any one of variety of immunological screening assays in which antibody competition can be assessed. A number of such assays are routinely practiced and well known in the art (see, e.g., U.S. Pat. No. 5,660,827, issued Aug. 26, 1997, which is incorporated herein by reference). It will be understood that determining the epitope to which an antibody described herein binds is not in any way required to identify an antibody that binds to the same or substantially the same epitope as the monoclonal antibody described herein.

For example, where the test antibodies to be examined are obtained from different source animals, or are even of a different Ig isotype, a simple competition assay may be employed in which the control antibody (for example, one of the antibodies disclosed in Tables 3-6) is mixed with the test antibody and then applied to a sample containing either or both SARS-COV-S or SARS-COV-2-S, each of which is known to be bound by the antibodies. Protocols based upon ELISAs, radioimmunoassays, Western blotting, and BIACORE® (GE Healthcare Life Sciences, Marlborough, MA) analysis (as described in the Examples section herein) are suitable for use in such simple competition studies.

In certain embodiments, the method comprises pre-mixing the control antibody with varying amounts of the test antibody (e.g., in ratios of about 1:1, 1:2, 1:10, or about 1:100) for a period of time prior to applying to the CoV-S antigen sample. In other embodiments, the control and varying amounts of test antibody can be added separately and admixed during exposure to the CoV-S antigen sample. As long as bound antibodies can be distinguished from free antibodies (e.g., by using separation or washing techniques to eliminate unbound antibodies) and control antibody from the test antibody (e.g., by using species specific or isotype specific secondary antibodies or by specifically labelling the control antibody with a detectable label), the method can be used to determine that the test antibody reduces the binding of the control antibody to the COV-S antigen, indicating that the test antibody recognizes substantially the same epitope as the control antibody (e.g., one of the antibodies disclosed in Tables 3-6). The binding of the (labeled) control antibody in the presence of a completely irrelevant antibody (that does not bind CoV-S) can serve as the control high value. The control low value can be obtained by incubating the labeled control antibody with the same but unlabeled control antibody, where competition would occur and reduce binding of the labeled antibody. In a test assay, a significant reduction in labeled antibody reactivity in the presence of a test antibody is indicative of a test antibody that recognizes substantially the same epitope, i.e., one that competes with the labeled control antibody. For example, any test antibody that reduces the binding of one or more antibodies disclosed in Tables 3-6, to both of SARS-COV-S or SARS-COV-2-S antigens by at least about 50%, such as at least about 60%, or more preferably at least about 70% (e.g., about 65-100%), at any ratio of control antibody:test antibody between about 1:1 or 1:10 and about 1:100 is considered to be an antibody that binds to substantially the same epitope or determinant as the antibody. Preferably, such test antibody will reduce the binding of one or more of the antibodies disclosed in Tables 3-6, to at least one, preferably each, of the SARS-COV-S or SARS-COV-2-S antigens preferably at least about 50%, at least about 60%, at least about 80% or at least about 90% (e.g., about 95%) of the binding of one or more of the antibodies disclosed in Tables 3-6, observed in the absence of the test antibody. These methods can be adapted to identify and/or evaluate antibodies that compete with other control antibodies.

A simple competition assay in which a test antibody is applied at saturating concentration to a surface onto which either SARS-COV-S or SARS-COV-2-S, or both, are immobilized also may be advantageously employed. The surface in the simple competition assay is preferably of a media suitable for OCTET® and/or PROTEON®. The binding of a control antibody (e.g., one or more antibodies disclosed in Tables 3-6) to the CoV-S-coated surface is measured. This binding to the CoV-S-containing surface of the control antibody alone is compared with the binding of the control antibody in the presence of a test antibody. A significant reduction in binding to the CoV-S-containing surface by the control antibody in the presence of a test antibody indicates that the test antibody recognizes substantially the same epitope as the control antibody such that the test antibody “competes” with the control antibody. Any test antibody that reduces the binding of control antibody (e.g., one or more antibodies disclosed in Tables 3-6) to both of SARS-COV-S and SARS-COV-2-S antigens by at least about 20% or more, at least about 40%, at least about 50%, at least about 70%, or more, can be considered to be an antibody that binds to substantially the same epitope or determinant as the control antibody (e.g., one or more antibodies disclosed in Tables 3-6). Preferably, such test antibody will reduce the binding of the control antibody (e.g., one or more antibodies disclosed in Tables 3-6) to the CoV-S antigen by at least about 50% (e.g., at least about 60%, at least about 70%, or more). It will be appreciated that the order of control and test antibodies can be reversed; i.e. the control antibody can be first bound to the surface and then the test antibody is brought into contact with the surface thereafter in a competition assay. Preferably, the antibody having higher affinity for SARS-COV-S and SARS-CoV-2-S is bound to the CoV-S-containing surface first, as it will be expected that the decrease in binding seen for the second antibody (assuming the antibodies are competing) will be of greater magnitude. Further examples of such assays are provided in, e.g., Saunal and Regenmortel, J. Immunol. Methods, 183:33-41 (1989), the disclosure of which is incorporated herein by reference.

Determination of whether an antibody, antigen-binding fragment thereof, or antibody derivative, e.g., an affinity-matured antibody or antigen binding fragment of any of the anti-CoV-S antibodies exemplified herein, binds within one of the epitope regions defined above can be carried out in ways known to the person skilled in the art. In another example of such mapping/characterization methods, an epitope region for an anti-CoV-S antibody may be determined by epitope “footprinting” using chemical modification of the exposed amines/carboxyls in the SARS-COV-S and SARS-COV-2-S protein. One specific example of such a foot-printing technique is the use of hydrogen-deuterium exchange detected by mass spectrometry (“HXMS”), wherein a hydrogen/deuterium exchange of receptor and ligand protein amide protons, binding, and back exchange occurs, wherein the backbone amide groups participating in protein binding are protected from back exchange and therefore will remain deuterated. Relevant regions can be identified at this point by peptic proteolysis, fast microbore high-performance liquid chromatography separation, and/or electrospray ionization mass spectrometry (See, e.g., Ehring H., Analytical Biochemistry, 267 (2): 252-259 (1999) and Engen, J. R. & Smith, D. L., Anal. Chem., 73: 256A-265A (2001)). Another example of a suitable epitope identification technique is nuclear magnetic resonance epitope mapping (“NMR”), where typically the position of the signals in two-dimensional NMR spectras of the free antigen and the antigen complexed with the antigen binding peptide, such as an antibody, are compared. The antigen typically is selectively isotopically labeled with ¹⁵N so that only signals corresponding to the antigen and no signals from the antigen binding peptide are seen in the NMR-spectrum. Antigen signals originating from amino acids involved in the interaction with the antigen binding peptide typically will shift position in the spectras of the complex compared to the spectras of the free antigen, and the amino acids involved in the binding can be identified that way. See, e.g., Ernst Schering Res. Found. Workshop, (44): 149-67 (2004); Huang et al., J. Mol. Biol., 281 (1): 61-67 (1998); and Saito and Patterson, Methods, 9 (3): 516-24 (1996). Epitope mapping/characterization also can be performed using mass spectrometry (“MS”) methods (See, e.g., Downard, J. Mass Spectrom., 35 (4): 493-503 (2000) and Kiselar and Downard, Anal. Chem., 71 (9): 1792-801 (1999)).

Protease digestion techniques also can be useful in the context of epitope mapping and identification. Antigenic determinant-relevant regions/sequences can be determined by protease digestion, e.g. by using trypsin in a ratio of about 1:50 to SARS-COV-S or SARS-COV-2-S overnight (“o/n”) digestion at 37° C. and pH 7-8, followed by mass spectrometry (“MS”) analysis for peptide identification. The peptides protected from trypsin cleavage by the anti-CoV-S antibody can subsequently be identified by comparison of samples subjected to trypsin digestion and samples incubated with antibody and then subjected to digestion by e.g. trypsin (thereby revealing a footprint for the antibody). Other enzymes like chymotrypsin or pepsin can be used in similar epitope characterization methods. Moreover, enzymatic digestion can provide a quick method for analyzing whether a potential antigenic determinant sequence is within a region of CoV-S in the context of a CoV-S-binding polypeptide. If the polypeptide is not surface exposed, it is most likely not relevant in terms of immunogenicity/antigenicity (See, e.g., Manca, Ann. 1st. Super. Sanità., 27 (1): 15-9 (1991) for a discussion of similar techniques).

Site-directed mutagenesis is another technique useful for characterization of a binding epitope. For example, in “alanine-scanning” site-directed mutagenesis (also known as alanine scanning, alanine scanning mutagenesis, alanine scanning mutations, combinatorial alanine scanning, or creation of alanine point mutations, for example), each residue within a protein segment is replaced with an alanine residue (or another residue such as valine where alanine is present in the wild-type sequence) through such methodologies as direct peptide or protein synthesis, site-directed mutagenesis, the GENEART™ Mutagenesis Service (Thermo Fisher Scientific, Waltham, MA U.S.A.) or shotgun mutagenesis, for example. A series of single point mutants of the molecule is thereby generated using this technique; the number of mutants generated is equivalent to the number of residues in the molecule, each residue being replaced, one at a time, by a single alanine residue. Alanine is generally used to replace native (wild-type) residues because of its non-bulky, chemically inert, methyl functional group that can mimic the secondary structure preferences that many other amino acids may possess. Subsequently, the effects replacing a native residue with an alanine has on binding affinity of an alanine scanning mutant and its binding partner can be measured using such methods as, but not limited to, SPR binding experiments. If a mutation leads to a significant reduction in binding affinity, it is most likely that the mutated residue is involved in binding. Monoclonal antibodies specific for structural epitopes (i.e., antibodies that do not bind the unfolded protein) can be used as a positive control for binding affinity experiments to verify that the alanine-replacement does not influence the overall tertiary structure of the protein (as changes to the overall fold of the protein may indirectly affect binding and thereby produce a false positive result). See, e.g., Clackson and Wells, Science, 267:383-386 (1995); Weiss et al., Proc. Natl. Acad. Sci. USA, 97 (16): 8950-8954 (2000); and Wells, Proc. Natl. Acad. Sci. USA, 93:1-6 (1996). Example 5 identifies the specific epitope or residues of CoV-S which specifically interact with the anti-CoV-S antibodies disclosed herein.

Electron microscopy can also be used for epitope “footprinting”. For example, Wang et al., Nature, 355:275-278 (1992) used coordinated application of cryoelectron microscopy, three-dimensional image reconstruction, and X-ray crystallography to determine the physical footprint of a Fab-fragment on the capsid surface of native cowpea mosaic virus.

Other forms of “label-free” assay for epitope evaluation include SPR (sold commercially as the BIACORE® system, GE Healthcare Life Sciences, Marlborough, MA) and reflectometric interference spectroscopy (“RifS”) (See, e.g., Fagerstam et al., Journal of Molecular Recognition, 3:208-14 (1990); Nice et al., J. Chromatogr., 646:159-168 (1993); Leipert et al., Angew. Chem. Int. Ed., 37:3308-3311 (1998); Kroger et al., Biosensors and Bioelectronics, 17:937-944 (2002)).

The expressions “framework region” or “FR” refer to one or more of the framework regions within the variable regions of the light and heavy chains of an antibody (See Kabat et al., Sequences of Proteins of Immunological Interest, 4^{th} edition, Bethesda, MD: U.S. Dept. of Health and Human Services, Public Health Service, National Institutes of Health (1987)). These expressions include those amino acid sequence regions interposed between the CDRs within the variable regions of the light and heavy chains of an antibody.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th edition, Bethesda, MD: U.S. Dept. of Health and Human Services, Public Health Service, National Institutes of Health (1991). The Fc region of an immunoglobulin generally comprises two constant domains, CH2 and CH3.

The terms “Fc receptor” and “FcR” describe a receptor that binds to the Fc region of an antibody. The preferred FcR is a native sequence human FcR. Moreover, a preferred FcR is one that binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. FcRs are reviewed in Ravetch and Kinet, Ann. Rev. Immunol., 9:457-92 (1991); Capel et al., Immunomethods, 4:25-34 (1994); and de Haas et al., J. Lab. Clin. Med., 126:330-41 (1995). “FcR” also includes the neonatal receptor, FcRn, which is responsible for the transfer of maternal IgGs to the fetus (Guyer et al., J. Immunol., 117:587 (1976); and Kim et al., J. Immunol., 24:249 (1994)), and which primarily functions to modulate and/or extend the half-life of antibodies in circulation. To the extent that the disclosed anti-CoV-S antibodies are aglycosylated, as a result of the expression system and/or sequence, the subject antibodies are expected to bind FcRn receptors, but not to bind (or to minimally bind) Fcγ receptors.

A “functional Fc region” possesses at least one effector function of a native sequence Fc region. Exemplary “effector functions” include C1q binding; complement dependent cytotoxicity (“CDC”); Fc receptor binding; antibody-dependent cell-mediated cytotoxicity (“ADCC”); phagocytosis; down-regulation of cell surface receptors (e.g. B cell receptor (“BCR”)), etc. Such effector functions generally require the Fc region to be combined with a binding domain (e.g. an antibody variable domain) and can be assessed using various assays known in the art for evaluating such antibody effector functions.

A “native sequence Fc region” comprises an amino acid sequence identical to the amino acid sequence of an Fc region found in nature. A “variant Fc region” comprises an amino acid sequence that differs from that of a native sequence Fc region by virtue of at least one amino acid modification, yet retains at least one effector function of the native sequence Fc region. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, more preferably at least about 95%, at least about 96%, at least about 97%, at least about 98%, or at least about 99% sequence identity therewith.

In some embodiments, the Fc region of an antibody or antigen-binding antibody fragment of the present disclosure may bind to an Fc receptor (FcR). The FcR may be, but is not limited to, Fc gamma receptor (FcgR), FcgRI, FcgRIIA, FcgRIIB1, FcgRIIB2, FcgRIIIA, FcgRIIIB, Fc epsilon receptor (FceR), FceRI, FceRII, Fc alpha receptor (FcaR), FcaRI, Fc alpha/mu receptor (Fca/mR), or neonatal Fc receptor (FcRn). The Fc may be an IgM, IgD, IgG, IgE, or IgA isotype. An IgG isotype may be an IgG1, IgG2, IgG3, or IgG4.

Certain amino acid modifications in the Fc region are known to modulate Ab effector functions and properties, such as, but not limited to, antibody-dependent cellular cytotoxicity (ADCC), antibody-dependent cellular phagocytosis (ADCP), complement dependent cytotoxicity (CDC), and half-life (Wang X. et al., Protein Cell. 2018 January; 9 (1): 63-73; Dall'Acqua W. F. et al., J Biol Chem. 2006 Aug. 18; 281 (33): 23514-24. Epub 2006 Jun. 21; Monnet C. et al, Front Immunol. 2015 Feb. 4; 6:39. doi: 10.3389/fimmu.2015.00039. eCollection 2015). The mutation may be symmetrical or asymmetrical. In certain cases, antibodies with Fc regions that have asymmetrical mutation(s) (i.e., two Fc regions are not identical) may provide better functions such as ADCC (Liu Z. et al. J Biol Chem. 2014 Feb. 7; 289 (6): 3571-3590).

Any of the antibody variable region sequences disclosed herein may be used in combination with a wild-type (WT) Fc or a variant Fc.

An IgG1-type Fc optionally may comprise one or more amino acid substitutions. Such substitutions may include, for example, N297A, N297Q, D265A, L234A, L235A, C226S, C229S, P238S, E233P, L234V, G236-deleted, P238A, A327Q, A327G, P329A, K322A, L234F, L235E, P331S, T394D, A330L, P331S, F243L, R292P, Y300L, V305I, P396L, S239D, 1332E, S298A, E333A, K334A, L234Y, L235Q, G236W, S239M, H268D, D270E, K326D, A330M, K334E, G236A, K326W, S239D, E333S, S267E, H268F, S324T, E345R, E430G, S440Y, M428L, N434S, L328F, M252Y, S254T, T256E, and/or any combination thereof (the residue numbering is according to the EU index as in Kabat) (Dall'Acqua W. F. et al., J Biol Chem. 2006 Aug. 18; 281 (33): 23514-24. Epub 2006 Jun. 21; Wang X. et al., Protein Cell. 2018 January; 9 (1): 63-73), or for example, N434A, Q438R, S440E, L432D, N434L, and/or any combination thereof (the residue numbering according to EU numbering). The Fc region may further comprise one or more additional amino acid substitutions. Such substitutions may include but are not limited to A330L, L234F, L235E, P3318, and/or any combination thereof (the residue numbering is according to the EU index as in Kabat). Specific exemplary substitution combinations for an IgG1-type Fc include, but not limited to: M252Y, S254T, and T256E (“YTE” variant); M428L and N434A (“LA” variant), M428L and N434S (“LS” variant); M428L, N434A, Q438R, and S440E (“LA-RE” variant); L432D and N434L (“DEL” variant); and L234A, L235A, L432D, and N434L (“LALA-DEL” variant) (the residue numbering is according to the EU index as in Kabat).

When the Ab is an IgG2, the Fc region optionally may comprise one or more amino acid substitutions. Such substitutions may include but are not limited to P238S, V234A, G237A, H268A, H268Q, H268E, V309L, N297A, N297Q, A330S, P331S, C232S, C233S, M252Y, S254T, T256E, and/or any combination thereof (the residue numbering is according to the EU index as in Kabat). The Fc region optionally may further comprise one or more additional amino acid substitutions. Such substitutions may include but are not limited to M252Y, S254T, T256E, and/or any combination thereof (the residue numbering is according to the EU index as in Kabat).

An IgG3-type Fc region optionally may comprise one or more amino acid substitutions. Such substitutions may include but are not limited to E235Y (the residue numbering is according to the EU index as in Kabat).

An IgG4-type Fc region optionally may comprise one or more amino acid substitutions. Such substitutions may include but are not limited to, E233P, F234V, L235A, G237A, E318A, S228P, L236E, S241P, L248E, T394D, M252Y, S254T, T256E, N297A, N297Q, and/or any combination thereof (the residue numbering is according to the EU index as in Kabat). The substitution may be, for example, S228P (the residue numbering is according to the EU index as in Kabat).

In some cases, the glycan of the human-like Fc region may be engineered to modify the effector function (for example, see Li T. et al., Proc Natl Acad Sci USA. 2017 Mar. 28; 114 (13):3485-3490. doi: 10.1073/pnas. 1702173114. Epub 2017 Mar. 13).

### B. Anti-CoV-S Antibodies and Binding Fragments Thereof Having Binding Activity for CoV-S

The present invention provides antibodies, or antigen-binding fragments thereof, that display broad activity against all SARS-COV-2 variants of concern (VOCs) described to date (including the BA.1/Omicron variant).

In some embodiments, an antibody or antigen-binding fragment thereof is capable of binding to the spike protein of a coronavirus (CoV-S). In some embodiments, the CoV-S is the spike protein of SARS-COV (“SARS-COV-S”) and/or the spike protein of SARS-COV-2 (“SARS-COV-2-S”).

In certain embodiments, an antibody or antigen-binding fragment thereof is capable of binding to a SARS-COV-2 variant. In some embodiments, the SARS-COV-2-S is a B.1.1.7 variant, a B. 1.351 variant, a B.1.1.28 variant, a B. 1.429 variant, a P.1 variant, a B.1.617 variant (e.g., B.1.617.1 and B.1.617.2), a C.37 variant, a 1.621 variant, a AY.1 variant, a 1.623 variant, a C.36 variant, a A.27 variant, a AV.1 variant, a B.1.1.482 variant, a B.1.1.523 variant, a B.1.427 variant, a AY.4 variant, a AY.11 variant, variant, a D614G variant, or a B.1.1.529/BA.1 variant (also known as the Omicron variant) and its sublineages (e.g., BA1.1, BA.2, BA.2.75, BA.4, BA.5, BA.4.6, BQ.1, BQ.1.1, XBB, XBB.1, XBB.1.5, BJ.1, BM.1.1.1, BA.2.3.20, BF.7, XBC, BN.1, or CH.1.1).

In some embodiments, the antibody or antigen-binding fragment thereof binds to the receptor binding domain (RBD) of CoV-S, e.g., the RBD from the B.1.1.529/BA.1 variant, the BF.7 variant, the BQ.1.1 variant, the BA.2.75 variant, the XBB.1 variant, the BA.2 variant, the B. 1.351 variant, the B.1.617 variant, or the D614G variant.

In some embodiments, the antibodies can be isolated from subjects experiencing SARS-COV-2, e.g., Omicron/BA.1, breakthrough infection. As used herein, the term “breakthrough infection” is defined as detection of SARS-COV-2 RNA or antigen in respiratory specimen collected from a person 14 days after receipt of all recommended doses of vaccines. As demonstrated in the Examples below, subjects experiencing BA.1 breakthrough infection were shown to have similar serum IgG binding titers to BA.1 and wild type CoV-S and RBD, whereas uninfected/vaccinated subjects displayed reduced serum IgG binding to BA.1 relative to WT CoV-S and RBD. In addition, subjects experiencing BA.1 breakthrough infection generally had higher serum IgA binding titers to BA.1 and WT RBD. Also, BA.1 breakthrough infection was shown to induce similar IgG+ B cell responses and higher magnitude IgA+ B cell responses to BA.1 RBD antigens relative to mRNA vaccination. BA.1 breakthrough infection also preferentially activates B cells that display cross-reactivity with both BA.1 and the original Wuhan-1 vaccine strain, and appears to re-direct B cell immunodominance hierarchy from the S2 subunit to the RBD. As shown in the Example, BA.1 breakthrough infection elicits RBD-directed antibodies with broad activity against SARS-COV-2 VOCs. These antibodies represent promising candidates for therapeutic development and provide a framework for the development of vaccines that induce broadly neutralizing antibody responses.

The antibodies are capable of binding to the spike protein of the coronavirus. CoV-S refers to the S protein of a coronavirus which is expressed on the surface of virions as a structural protein. As mentioned previously, the S protein plays an essential role for coronaviruses in binding to receptors on the host cell and determines host tropism (Zhu Z. et al., Infect Genet Evol. 2018 July; 61:183-184. doi: 10.1016/j.meegid.2018.03.028. Epub 2018 Apr. 4). SARS-COV and SARS-COV-2 bind to angiotensin-converting enzyme 2 (ACE2) of the host cell via the S protein's receptor-binding domains (RBDs) and uses ACE2 as a receptor to enter the host cells (Ge X. Y. et al., Nature. 2013 Nov. 28; 503 (7477): 535-8. doi: 10.1038/nature12711. Epub 2013 Oct. 30.; Hoffmann M. et al., Cell. 2020 Mar. 4. pii: S0092-8674 (20) 30229-4. doi: 10.1016/j.cell.2020.02.052). SARS-COV can also use CD209L (also known as L-SIGN) as an alternative receptor (Jeffers S. A. et al., Proc Natl Acad Sci USA. 2004 Nov. 2; 101 (44): 15748-53. Epub 2004 Oct. 20). MERS-COV binds dipeptidyl peptidase 4 (“DPP4”, also known as CD26) of the host cells via a different RBD of the S protein. Cell entry of coronaviruses depends on not only binding of the S protein to a host cell receptor but often also priming of the S protein by host cell proteases, and recently SARS-COV-2 was found to use the serine protease TMPRSS2 for S protein priming and then ACE2 for entry (Wu A. et al., Cell Host Microbe. 2020 Mar. 11; 27 (3): 325-328. doi: 10.1016/j.chom.2020.02.001. Epub 2020 Feb. 7; Hoffmann M. et al., Cell. 2020 Mar. 4. pii: S0092-8674 (20) 30229-4. doi: 10.1016/j.cell.2020.02.052).

The S protein of SARS-COV is referred to as SARS-COV-S and may for example comprise the amino acid sequence of SEQ ID NO: 1 (1288 amino acids). The S protein of SARS-COV-2 is referred to as SARS-COV-2-S and may for example comprise the amino acid sequence of SEQ ID NO: 5 (1273 amino acids).

The present disclosure provides exemplary antibodies and antigen-binding antibody fragments that bind, e.g., specifically bind, to CoV, wherein at least some of these antibodies and antigen-binding antibody fragments bind to SARS-COV-2-S and/or SARS-COV-2-S. Due to the sequence similarity among different CoV species, such antibodies or antigen-binding antibody fragments of the present disclosure may also cross react with the S protein of other CoV species.

The exemplary S proteins of CoV that the antibodies or antigen-binding antibody fragments of the present disclosure may bind, e.g., specifically bind, include by way of example, Bat SARS COV (GenBank Accession No. FJ211859), SARS COV (GenBank Accession No. FJ211860), BtSARS.HKU3.1 (GenBank Accession No. DQ022305), BtSARS.HKU3.2 (GenBank Accession No. DQ084199), BtSARS.HKU3.3 (GenBank Accession No. DQ084200), BtSARS.Rml (GenBank Accession No. DQ412043), BtCoV.279.2005 (GenBank Accession No. DQ648857), BtSARS.Rf1 (GenBank Accession No. DQ412042), BtCoV.273.2005 (GenBank Accession No. DQ648856), BtSARS.Rp3 (GenBank Accession No. DQ071615), SARS COV.A022 (GenBank Accession No. AY686863), SARSCOV.CUHK-W1 (GenBank Accession No. AY278554), SARSCOV.GDO1 (GenBank Accession No. AY278489), SARSCOV.HC.SZ.61.03 (GenBank Accession No. AY515512), SARSCOV.SZ16 (GenBank Accession No. AY304488), SARSCOV.Urbani (GenBank Accession No. AY278741), SARSCOV.civet010 (GenBank Accession No. AY572035), or SARSCOV.MA.15 (GenBank Accession No. DQ497008), Rs SHC014 (GenBank® Accession No. KC881005), Rs3367 (GenBank® Accession No. KC881006), WiV1 S (GenBank® Accession No. KC881007).

In some embodiments, the antibodies and antigen-binding antibody fragments provided herein may also bind to and neutralize existing bat CoV or pre-emergent bat CoVs. Antibodies and antigen-binding antibody fragments with such binding and/or neutralization abilities would be particularly useful in a future pandemic that may be caused by a spillover from an animal reservoir, like a bat.

Alternatively, the S proteins of CoV to which the antibodies or antigen-binding antibody fragments of the present disclosure may bind, e.g., specifically bind, to and neutralize pre-emergent coronaviruses from other species, e.g., bats.

Still alternatively, the S proteins of CoV to which the antibodies or antigen-binding antibody fragments of the present disclosure may bind, e.g., specifically bind, to may include, for example, Middle East respiratory syndrome coronavirus isolate Riyadh_2_2012 (GenBank Accession No. KF600652.1), Middle East respiratory syndrome coronavirus isolate Al-Hasa_18_2013 (GenBank Accession No. KF600651.1), Middle East respiratory syndrome coronavirus isolate Al-Hasa_17_2013 (GenBank Accession No. KF600647.1), Middle East respiratory syndrome coronavirus isolate Al-Hasa_15_2013 (GenBank Accession No. KF600645.1), Middle East respiratory syndrome coronavirus isolate Al-Hasa_16_2013 (GenBank Accession No. KF600644.1), Middle East respiratory syndrome coronavirus isolate Al-Hasa_21_2013 (GenBank Accession No. KF600634), Middle East respiratory syndrome coronavirus isolate Al-Hasa_19_2013 (GenBank Accession No. KF600632), Middle East respiratory syndrome coronavirus isolate Buraidah_1_2013 (GenBank Accession No. KF600630.1), Middle East respiratory syndrome coronavirus isolate Hafr-Al-Batin_1_2013 (GenBank Accession No. KF600628.1), Middle East respiratory syndrome coronavirus isolate Al-Hasa_12_2013 (GenBank Accession No. KF600627.1), Middle East respiratory syndrome coronavirus isolate Bisha_1_2012 (GenBank Accession No. KF600620.1), Middle East respiratory syndrome coronavirus isolate Riyadh_3_2013 (GenBank Accession No. KF600613.1), Middle East respiratory syndrome coronavirus isolate Riyadh_1_2012 (GenBank Accession No. KF600612.1), Middle East respiratory syndrome coronavirus isolate Al-Hasa_3_2013 (GenBank Accession No. KF186565.1), Middle East respiratory syndrome coronavirus isolate Al-Hasa_1_2013 (GenBank Accession No. KF186567.1), Middle East respiratory syndrome coronavirus isolate Al-Hasa_2_2013 (GenBank Accession No. KF186566.1), Middle East respiratory syndrome coronavirus isolate Al-Hasa_4_2013 (GenBank Accession No. KF186564.1), Middle East respiratory syndrome coronavirus (GenBank Accession No. KF192507.1), Betacoronavirus England 1-N1 (GenBank Accession No. NC_019843), MERS-COV_SA-N1 (GenBank Accession No. KC667074), following isolates of Middle East Respiratory Syndrome Coronavirus (GenBank Accession No: KF600656.1, GenBank Accession No: KF600655.1, GenBank Accession No: KF600654.1, GenBank Accession No: KF600649.1, GenBank Accession No: KF600648.1, GenBank Accession No: KF600646.1, GenBank Accession No: KF600643.1, GenBank Accession No: KF600642.1, GenBank Accession No: KF600640.1, GenBank Accession No: KF600639.1, GenBank Accession No: KF600638.1, GenBank Accession No: KF600637.1, GenBank Accession No: KF600636.1, GenBank Accession No: KF600635.1, GenBank Accession No: KF600631.1, GenBank Accession No: KF600626.1, GenBank Accession No: KF600625.1, GenBank Accession No: KF600624.1, GenBank Accession No: KF600623.1, GenBank Accession No: KF600622.1, GenBank Accession No: KF600621.1, GenBank Accession No: KF600619.1, GenBank Accession No: KF600618.1, GenBank Accession No: KF600616.1, GenBank Accession No: KF600615.1, GenBank Accession No: KF600614.1, GenBank Accession No: KF600641.1, GenBank Accession No: KF600633.1, GenBank Accession No: KF600629.1, GenBank Accession No: KF600617.1), Coronavirus Neoromicia/PML-PHE1/RSA/2011 GenBank Accession: KC869678.2, Bat Coronavirus Taper/CII_KSA_287/Bisha/Saudi Arabia/GenBank Accession No: KF493885.1, Bat coronavirus Rhhar/CII_KSA_003/Bisha/Saudi Arabia/2013 GenBank Accession No: KF493888.1, Bat coronavirus Pikuh/CII_KSA_001/Riyadh/Saudi Arabia/2013 GenBank Accession No: KF493887.1, Bat coronavirus Rhhar/CII_KSA_002/Bisha/Saudi Arabia/2013 GenBank Accession No: KF493886.1, Bat Coronavirus Rhhar/CII_KSA_004/Bisha/Saudi Arabia/2013 GenBank Accession No: KF493884.1, BtCoV.HKU4.2 (GenBank Accession No. EF065506), BtCoV.HKU4.1 (GenBank Accession No. NC_009019), BtCoV.HKU4.3 (GenBank Accession No. EF065507), BtCoV.HKU4.4 (GenBank Accession No. EF065508), BtCoV 133.2005 (GenBank Accession No. NC 008315), BtCoV.HKU5.5 (GenBank Accession No. EF065512); BtCoV.HKU5.1 (GenBank Accession No. NC_009020), BtCoV.HKU5.2 (GenBank Accession No. EF065510), BtCoV.HKU5.3 (GenBank Accession No. EF065511), human betacoronavirus 2c Jordan-N3/2012 (GenBank Accession No. KC776174.1; human betacoronavirus 2c EMC/2012 (GenBank Accession No. JX869059.2), Pipistrellus bat coronavirus HKU5 isolates (GenBank Accession No: KC522089.1, GenBank Accession No: KC522088.1, GenBank Accession No: KC522087.1, GenBank Accession No: KC522086.1, GenBank Accession No: KC522085.1, GenBank Accession No: KC522084.1, GenBank Accession No: KC522083.1, GenBank Accession No: KC522082.1, GenBank Accession No: KC522081.1, GenBank Accession No: KC522080.1, GenBank Accession No: KC522079.1, GenBank Accession No: KC522078.1, GenBank Accession No: KC522077.1, GenBank Accession No: KC522076.1, GenBank Accession No: KC522075.1, GenBank Accession No: KC522104.1, GenBank Accession No: KC522104.1, GenBank Accession No: KC522103.1, GenBank Accession No: KC522102.1, GenBank Accession No: KC522101.1, GenBank Accession No: KC522100.1, GenBank Accession No: KC522099.1, GenBank Accession No: KC522098.1, GenBank Accession No: KC522097.1, GenBank Accession No: KC522096.1, GenBank Accession No: KC522095.1, GenBank Accession No: KC522094.1, GenBank Accession No: KC522093.1, GenBank Accession No: KC522092.1, GenBank Accession No: KC522091.1, GenBank Accession No: KC522090.1, GenBank Accession No: KC522119.1 GenBank Accession No: KC522118.1 GenBank Accession No: KC522117.1 GenBank Accession No: KC522116.1 GenBank Accession No: KC522115.1 GenBank Accession No: KC522114.1 GenBank Accession No: KC522113.1 GenBank Accession No: KC522112.1 GenBank Accession No: KC522111.1 GenBank Accession No: KC522110.1 GenBank Accession No: KC522109.1 GenBank Accession No: KC522108.1, GenBank Accession No: KC522107.1, GenBank Accession No: KC522106.1, GenBank Accession No: KC522105.1) Pipistrellus bat coronavirus HKU4 isolates (GenBank Accession No: KC522048.1, GenBank Accession No: KC522047.1, GenBank Accession No: KC522046.1, GenBank Accession No: KC522045.1, GenBank Accession No: KC522044.1, GenBank Accession No: KC522043.1, GenBank Accession No: KC522042.1, GenBank Accession No: KC522041.1, GenBank Accession No: KC522040.1 GenBank Accession No: KC522039.1, GenBank Accession No: KC522038.1, GenBank Accession No: KC522037.1, GenBank Accession No: KC522036.1, GenBank Accession No: KC522048.1 GenBank Accession No: KC522047.1 GenBank Accession No: KC522046.1 GenBank Accession No: KC522045.1 GenBank Accession No: KC522044.1 GenBank Accession No: KC522043.1 GenBank Accession No: KC522042.1 GenBank Accession No: KC522041.1 GenBank Accession No: KC522040.1, GenBank Accession No: KC522039.1 GenBank Accession No: KC522038.1 GenBank Accession No: KC522037.1 GenBank Accession No: KC522036.1, GenBank Accession No: KC522061.1 GenBank Accession No: KC522060.1 GenBank Accession No: KC522059.1 GenBank Accession No: KC522058.1 GenBank Accession No: KC522057.1 GenBank Accession No: KC522056.1 GenBank Accession No: KC522055.1 GenBank Accession No: KC522054.1 GenBank Accession No: KC522053.1 GenBank Accession No: KC522052.1 GenBank Accession No: KC522051.1 GenBank Accession No: KC522050.1 GenBank Accession No: KC522049.1 GenBank Accession No: KC522074.1, GenBank Accession No: KC522073.1 GenBank Accession No: KC522072.1 GenBank Accession No: KC522071.1 GenBank Accession No: KC522070.1 GenBank Accession No: KC522069.1 GenBank Accession No: KC522068.1 GenBank Accession No: KC522067.1, GenBank Accession No: KC522066.1 GenBank Accession No: KC522065.1 GenBank Accession No: KC522064.1, GenBank Accession No: KC522063.1, or GenBank Accession No: KC522062.1.

Alternatively, the S proteins of CoV to which the antibodies or antigen-binding antibody fragments of the present disclosure may bind, e.g., specifically bind, may include for example, FCov.FIPV.79.1146.VR.2202 (GenBank Accession No. NV_007025), transmissible gastroenteritis virus (TGEV) (GenBank Accession No. NC_002306; GenBank Accession No. Q811789.2; GenBank Accession No. DQ811786.2; GenBank Accession No. DQ811788.1; GenBank Accession No. DQ811785.1; GenBank Accession No. X52157.1; GenBank Accession No. AJ011482.1; GenBank Accession No. KC962433.1; GenBank Accession No. AJ271965.2; GenBank Accession No. JQ693060.1; GenBank Accession No. KC609371.1; GenBank Accession No. JQ693060.1; GenBank Accession No. JQ693059.1; GenBank Accession No. JQ693058.1; GenBank Accession No. JQ693057.1; GenBank Accession No. JQ693052.1; GenBank Accession No. JQ693051.1; GenBank Accession No. JQ693050.1), or porcine reproductive and respiratory syndrome virus (PRRSV) (GenBank Accession No. NC_001961.1; GenBank Accession No. DQ811787).

Alternatively, the S proteins of CoV to which the antibodies or antigen-binding antibody fragments of the present disclosure may bind, e.g., specifically bind, may include, for example, BtCoV.1A.AFCD62 (GenBank Accession No. NC_010437), BtCoV.1B.AFCD307 (GenBank Accession No. NC_010436), BtCov.HKU8.AFCD77 (GenBank Accession No. NC_010438), BtCoV.512.2005 (GenBank Accession No. DQ648858), porcine epidemic diarrhea virus PEDV.CV777 (GenBank Accession No. NC_003436, GenBank Accession No. DQ355224.1, GenBank Accession No. DQ355223.1, GenBank Accession No. DQ355221.1, GenBank Accession No. JN601062.1, GenBank Accession No. N601061.1, GenBank Accession No. JN601060.1, GenBank Accession No. JN601059.1, GenBank Accession No. JN601058.1, GenBank Accession No. JN601057.1, GenBank Accession No. JN601056.1, GenBank Accession No. JN601055.1, GenBank Accession No. JN601054.1, GenBank Accession No. JN601053.1, GenBank Accession No. JN601052.1, GenBank Accession No. JN400902.1, GenBank Accession No. JN547395.1, GenBank Accession No. FJ687473.1, GenBank Accession No. FJ687472.1, GenBank Accession No. FJ687471.1, GenBank Accession No. FJ687470.1, GenBank Accession No. FJ687469.1, GenBank Accession No. FJ687468.1, GenBank Accession No. FJ687467.1, GenBank Accession No. FJ687466.1, GenBank Accession No. FJ687465.1, GenBank Accession No. FJ687464.1, GenBank Accession No. FJ687463.1, GenBank Accession No. FJ687462.1, GenBank Accession No. FJ687461.1, GenBank Accession No. FJ687460.1, GenBank Accession No. FJ687459.1, GenBank Accession No. FJ687458.1, GenBank Accession No. FJ687457.1, GenBank Accession No. FJ687456.1, GenBank Accession No. FJ687455.1, GenBank Accession No. FJ687454.1, GenBank Accession No. FJ687453 GenBank Accession No. FJ687452.1, GenBank Accession No. FJ687451.1, GenBank Accession No. FJ687450.1, GenBank Accession No. FJ687449.1, GenBank Accession No. AF500215.1, GenBank Accession No. KF476061.1, GenBank Accession No. KF476060.1, GenBank Accession No. KF476059.1, GenBank Accession No. KF476058.1, GenBank Accession No. KF476057.1, GenBank Accession No. KF476056.1, GenBank Accession No. KF476055.1, GenBank Accession No. KF476054.1, GenBank Accession No. KF476053.1, GenBank Accession No. KF476052.1, GenBank Accession No. KF476051.1, GenBank Accession No. KF476050.1, GenBank Accession No. KF476049.1, GenBank Accession No. KF476048.1, GenBank Accession No. KF177258.1, GenBank Accession No. KF177257.1, GenBank Accession No. KF177256.1, GenBank Accession No. KF177255.1), HCoV.229E (GenBank Accession No. NC_002645), HCoV.NL63.Amsterdam.I (GenBank Accession No. NC_005831), BtCoV.HKU2.HK.298.2006 (GenBank Accession No. EF203066), BtCoV.HKU2.HK.33.2006 (GenBank Accession No. EF203067), BtCoV.HKU2.HK.46.2006 (GenBank Accession No. EF203065), or BtCoV.HKU2.GD.430.2006 (GenBank Accession No. EF203064).

Alternatively, the S proteins of CoV to which the antibodies or antigen-binding antibody fragments of the present disclosure may bind, e.g., specifically bind, may include, for example, HCoV.HKU1.C.N5 (GenBank Accession No. DQ339101), MHV.A59 (GenBank Accession No. NC 001846), PHEV.VW572 (GenBank Accession No. NC 007732), HCoV.OC43.ATCC.VR.759 (GenBank Accession No. NC_005147), or bovine enteric coronavirus (BCoV.ENT) (GenBank Accession No. NC_003045).

Alternatively, the S proteins of CoV to which the antibodies or antigen-binding antibody fragments of the present disclosure may bind, e.g., specifically bind, may include, for example, BtCoV.HKU9.2 (GenBank Accession No. EF065514), BtCoV.HKU9.1 (GenBank Accession No. NC_009021), BtCoV.HkU9.3 (GenBank Accession No. EF065515), or BtCoV.HKU9.4 (GenBank Accession No. EF065516).sarbecovirus

In some instances, an anti-CoV-S antibody or antigen-binding fragment thereof according to the disclosure binds to CoV-S (e.g., SARS-COV-S and/or SARS-COV-2-S, and/or any of the CoV S proteins listed above) with a dissociation constant (KD) of (i) 100 nM or lower; (ii) about 10 nM or lower; (iii) about 1 nM or lower; (iv) about 100 pM or lower; (v) about 10 pM or lower; (vi) about 1 pM or lower; or (vii) about 0.1 pM or lower.

In some instances, an anti-CoV-S antibody or antigen-binding fragment thereof according to the disclosure binds to the RBD of CoV-S (e.g., SARS-COV-S and/or SARS-COV-2-S, and/or any of the CoV S proteins listed above) with a dissociation constant (KD) of (i) 100 nM or lower; (ii) about 10 nM or lower; (iii) about 1 nM or lower; (iv) about 100 pM or lower; (v) about 10 pM or lower; (vi) about 1 pM or lower; or (vii) about 0.1 pM or lower.

In some instances, an anti-CoV-S antibody or antigen-binding fragment thereof according to the disclosure binds to the RBD of CoV-S of the B.1.1.529/BA.1 variant, the BF.7 variant, the BQ. 1.1 variant, the BA.2.75 variant, the XBB.1 variant, the B.1351 variant, or the B.1.617.2 variant, with a dissociation constant (KD) of (i) 100 nM or lower; (ii) about 10 nM or lower; (iii) about 1 nM or lower; (iv) about 100 pM or lower; (v) about 10 pM or lower; (vi) about 1 pM or lower; or (vii) about 0.1 pM or lower.

The present disclosure provides exemplary antibodies or antigen-binding fragments thereof that bind CoV-S, including human CoV-S, which optionally may be affinity-matured. Other antibodies or antigen-binding fragments thereof that bind CoV-S, including those having different CDRs, and epitopic specificity may be obtained using the disclosure of the present specification, and using methods that are generally known in the art. Such antibodies and antigen-binding fragments thereof antagonize the biological effects of CoV-S in vivo and therefore are useful in treating or preventing COV-S-related conditions including, particularly coronavirus infection. In preferred embodiments, the antibody or antigen-binding fragment thereof according to the disclosure comprises one or more CDRs, a V_{L} chain and/or V_{H} chain of the anti-CoV-S antibodies and antigen-binding fragments thereof described herein.

In some embodiments, an anti-CoV-S antibody or antigen-binding fragment thereof according to the disclosure will interfere with, block, reduce, or modulate the interaction between COV-S and its receptor(s) (e.g., ACE2, CD209L, L-SIGN, DPP4, or CD26) on host cells or a S protein-priming protein on host cells (e.g., TMPRSS2). If binding of the S protein to its receptor is blocked or reduced, CoV virions may be prohibited from entering the cells, i.e., infection to further cells is prevented. Also, if the S protein is prevented from binding to a S protein-priming protein, the S protein would not be activated and therefore the host cell entry via the receptor may be reduced, i.e., infection to further cells is prevented.

In some instance, an anti-CoV-S antibody or antigen-binding fragment thereof according to the disclosure is “neutralizing”, e.g., it substantially or totally prevents the specific interaction of CoV-S with the host receptors or priming protein. As a result, CoV virions may be substantially or totally cleared by immune cells of the host, such as phagocytes via, for example, Fc receptor mediated phagocytosis or mere phagocytosis due to increased time of virions outside the cells. In some embodiments, the antibody or antigen-binding fragment thereof neutralizes CoV-S, e.g., by remaining bound to CoV-S in a location and/or manner that prevents CoV-S from binding to its receptor or priming protein on host cells. As a result, CoV virions may be substantially or totally prevented from entering the cells, i.e. infection to further cells is prevented.

In certain embodiments, an anti-CoV-S antibody or antigen-binding fragment thereof according to the disclosure neutralizes CoV (e.g., SARS-COV and/or SARS-COV-2) at an IC50 of about 100 nM or lower, of about 50 nM or lower, of about 20 nM or lower, of about 10 nM or lower, of about 5 nM or lower, of about 2 nM or lower, of about 1 nM or lower, of about 500 pM or lower, of about 200 pM or lower, of about 100 pM or lower, of about 50 pM or lower, of about 20 pM or lower, of about 10 pM or lower, of about 5 pM or lower, of about 2 pM or lower, or of about 1 pM or lower, or at an IC50 of about 500 ng/mL or lower, of about 200 ng/ml or lower, of about 100 ng/ml or lower, of about 50 ng/ml or lower, at about 20 ng/mL or lower, at about 10 ng/ml or lower, at about 20 ng/ml or lower, at about 10 mg/mL or lower, at about 5 ng/ml or lower, at about 2 ng/ml or lower, or at about 1 ng/ml or lower, in vitro, as measured by any of the neutralization assays described in Examples herein.

In some embodiments, the antibody, or antigen-binding fragment thereof, neutralizes the B.1.1.529/BA.1 variant of SARS-COV-2 with an IC50 of about 100 ng/ml or lower, of about 50 ng/ml or lower, of about 40 ng/ml or lower, of about 30 ng/mL or lower, of about 20 ng/ml or lower, of about 10 mg/mL or lower, of about 5 ng/ml or lower, of about 2 ng/mL or lower, or of about 1 ng/ml or lower, in vitro. In some embodiments, the antibody, or antigen-binding fragment thereof, neutralizes the B.1.1.529/BA.1 variant of SARS-COV-2 with an IC50 of about 60 ng/mL or lower.

In some embodiments, the antibody, or antigen-binding fragment thereof, neutralizes the BA2.75 variant, the BF.7 variant, the BQ.1.1 variant, the XBB.1 variant, and/or the XBB.1.5 of SARS-CoV-2 with an IC50 of about 200 ng/mL or lower, of about 100 ng/ml or lower, of about 50 ng/ml or lower, of about 40 ng/mL or lower, of about 30 ng/ml or lower, of about 20 ng/ml or lower, of about 10 mg/mL or lower, of about 5 ng/ml or lower, of about 2 ng/ml or lower, or of about 1 ng/ml or lower, in vitro.

In some embodiments, the antibody, or antigen-binding fragment thereof, neutralizes the BA.2.75 variant of SARS-COV-2 with an IC50 of about 40 ng/ml or lower.

In some embodiments, the antibody, or antigen-binding fragment thereof, neutralizes the BF.7 variant of SARS-COV-2 with an IC50 of about 30 ng/mL or lower.

In some embodiments, the antibody, or antigen-binding fragment thereof, neutralizes the BQ.1.1 variant of SARS-COV-2 with an IC50 of about 50 ng/mL or lower.

In some embodiments, the antibody, or antigen-binding fragment thereof, neutralizes the XBB.1 variant of SARS-COV-2 with an IC50 of about 200 ng/ml or lower.

In some embodiments, the antibody, or antigen-binding fragment thereof, neutralizes the D614G variant of SARS-COV-2 with an IC50 of about 100 ng/ml or lower, of about 50 ng/ml or lower, of about 40 ng/ml or lower, of about 30 ng/ml or lower, of about 20 ng/ml or lower, of about 10 mg/mL or lower, of about 5 ng/ml or lower, of about 2 ng/ml or lower, or of about 1 ng/mL or lower, in vitro. In some embodiments, the antibody, or antigen-binding fragment thereof, neutralizes the D614G variant of SARS-COV-2 with an IC50 of about 20 ng/ml or lower,

In some instances, an anti-CoV-S antibody or antigen-binding fragment thereof according to the disclosure or cocktail thereof, when administered to a coronavirus infected host or one susceptible to coronavirus infection such as a health care worker may promote a neutralization response in the host against the coronavirus which is sufficient to permit the host to be able to mount an effective cell-mediated immune response against the virus, e.g., T cell-mediated or cytokine-mediated immune response against the coronavirus and/or to be more responsive to other treatment methods such as drugs, antivirals or other biologics.

As mentioned, the anti-CoV-S antibodies or antigen-binding fragments thereof according to the disclosure have a variety of uses. For example, the subject antibodies and fragments can be useful in prophylactic or therapeutic applications, as well as diagnostically in binding assays. The subject anti-CoV-S antibodies or antigen-binding fragments thereof are useful for affinity purification of CoV-S, in particular human CoV-S or its ligands and in screening assays to identify other antagonists of CoV-S activity. Some of the antibodies or antigen-binding fragments thereof are useful for inhibiting binding of CoV-S to its receptor(s) (e.g., ACE2, CD209L, L-SIGN, DPP4, or CD26) on host cells or a S protein-priming protein on host cells (e.g., TMPRSS2) or inhibiting COV-S-mediated activities and/or biological effects.

As used herein, the term “one or more biological effects associated with COV-S refers to any biological effect mediated, induced, or otherwise attributable to COV-S, e.g., binding properties, functional properties, and other properties of biological significance. Non-limiting exemplary biological effects of COV-S include COV-S binding to its receptor(s) (e.g., ACE2, CD209L, L-SIGN, DPP4, or CD26) on host cells or a S protein-priming protein on host cells (e.g., TMPRSS2), activation of host cells for allowing virus entry, activation of immune cells as a result of the entry of CoV into the cell, e.g., via presentation of CoV antigen(s) on the host cells' MHC molecule, and resulting inflammation. The subject anti-CoV-S antibodies are capable of inhibiting one, a combination of, or all of these exemplary CoV-S biological activities. For example, the anti-CoV-S antibodies and antigen-binding fragments thereof provided herein may neutralize CoV virions or reduce the infectivity of CoV virions.

The antibody or antigen-binding fragment thereof according to the disclosure can be used in a variety of therapeutic applications. For example, in some embodiments the anti-CoV-S antibody or antigen-binding fragment thereof are useful for treating conditions associated with CoV-S, such as, but not limited to, symptoms associated with CoV infection. The CoV may be any CoV, including SARS-CoV, SARS-COV-2, MERS-COV, HCoV-HKU1, HCoV-OC43, HCoV-229E, and HCoV-NL63, and also may be any of the CoV species listed above herein.

Specific examples of CoV infection-associated symptoms are fever, cough, dry cough, shortness of breath or difficulty of breath, fatigue, aches, runny nose, congestion, sore throat, conjunctivitis, chest pain, headache, muscle ache, chills, loss of smell, and loss of taste, and gastrointestinal symptoms including diarrhea. Complications and/or diseases/disorders associated with coronavirus infection may include, for example, bronchitis, pneumonia, respiratory failure, acute respiratory failure, organ failure, multi-organ system failure, pediatric inflammatory multisystem syndrome, acute respiratory distress syndrome (a severe lung condition that causes low oxygen in the blood and organs), blood clots, cardiac conditions, myocardial injury, myocarditis, heart failure, cardiac arrest, acute myocardial infarction, dysrhythmias, venous thromboembolism, post-intensive care syndrome, shock, anaphylactic shock, cytokine release syndrome, septic shock, disseminated intravascular coagulation, ischemic stroke, intracerebral hemorrhage, microangiopathic thrombosis, psychosis, seizure, nonconvulsive status epilepticus, traumatic brain injury, stroke, anoxic brain injury, encephalitis, posterior reversible leukoencephalopathy, necrotizing encephalopathy, post-infectious encephalitis, autoimmune mediated encephalitis, acute disseminated encephalomyelitis, acute kidney injury, acute liver injury, pancreatic injury, immune thrombocytopenia, subacute thyroiditis, gastrointestinal complications, aspergillosis, increased susceptibility to infection with another virus or bacteria, and/or pregnancy-related complications. Certain diseases and conditions, such as high blood pressure, type 1 diabetes, liver disease, overweight, chronic lung diseases including cystic fibrosis, pulmonary fibrosis, and asthma, compromised immune system due to transplant, use of an immunosuppressant, or HIV infection, and brain and nervous system condition, may increase the risk of CoV infection-associated complications and diseases.

The subject anti-CoV-S antibodies and antigen-binding fragments thereof may be used alone or in association with other active agents or drugs, including other biologics, to treat any subject in which blocking, inhibiting, or neutralizing the in vivo effect of CoV-S or blocking or inhibiting the interaction of CoV-S and its receptor(s) (e.g., ACE2, CD209L, L-SIGN, DPP4, or CD26) on host cells or a S protein-priming protein on host cells (e.g., TMPRSS2), is therapeutically desirable.

The anti-CoV-S antibodies and antigen-binding fragments thereof comprising the disclosure have binding affinity for CoV-S, such as SARS-COV-S or SARS-COV-S2. Some antibodies of the present disclosure binds to SARS-COV-S or SARS-COV-S2 with a similar K_{D} (M), while some antibodies of the present disclosure bind to SARS-COV-S with a lower K_{D} (M) (i.e., higher affinity) than to SARS-COV-S2, and some antibodies of the present disclosure bind to SARS-COV-S-2 with a lower K_{D} (M) (i.e., higher affinity) than to SARS-COV-S.

Exemplary anti-CoV antibodies and antigen-binding fragments thereof according to the disclosure, and the specific CDRs thereof are identified in this section. For convenience, each exemplified antibody or antigen-binding fragment thereof, and corresponding sequences are separately identified by a specific nomenclature as shown in Tables 3-6.

The anti-CoV-S antibodies and antigen-binding fragments thereof comprising the disclosure have binding affinity for CoV-S, such as SARS-COV-S or SARS-COV-S2. Some antibodies of the present disclosure binds to SARS-COV-S or SARS-COV-S2 with a similar K_{D} (M), while some antibodies of the present disclosure bind to SARS-COV-S with a lower K_{D} (M) (i.e., higher affinity) than to SARS-COV-S2, and some antibodies of the present disclosure bind to SARS-COV-S-2 with a lower K_{D} (M) (i.e., higher affinity) than to SARS-COV-S. Affinities to different CoV-S proteins for antibodies of the present disclosure are provided in Tables 3-6.

### C. Anti-CoV-S Antibody Polypeptide Sequences and Nucleic Acid Sequences Encoding Thereof

### Antibodies Disclosed Herein

Anti-CoV-S antibodies, and antigen-binding fragments thereof, specifically provided by the present disclosure include any one of the antibodies as shown Tables 3-6, and antigen-binding fragments thereof. Any Fc variant may be used in combination with any of the variable sequences disclosed herein.

Tables 3 and 5 (Appendixes A and C) and Tables 4 and 6 (Appendixes B and D) show (i) the amino acid sequences of the VH, VH FR1, VH CDR1, VH FR2, VH CDR2, VH FR3, VH CDR3, VH FR4, VL, VL FR1, VL CDR1, VL FR2, VL CDR2, VL FR3, VL CDR3, and VL FR4 for individual antibodies, and (ii) the DNA sequences of the VH and VL chains for individual antibodies. In some embodiments, the VH comprises a reverted VH sequence of an antibody. In some embodiments, the VL sequence comprises a reverted VL sequence of an antibody. The reverted VH and VL sequences include reversions of primer-induced mutations to germline sequences, and also include silent amino acid mutations that have been reverted to germinline DNA codons.

### Variations of the Disclosed Antibodies and Polynucleotide Sequences Encoding Such Variations

In one embodiment, the disclosure contemplates anti-CoV-S antibodies or antigen-binding antibody fragments comprising (i) a VH CDR that is same as the VH CDR3 of, (ii) a VH CDR3 and VL CDR3, both of which as same as both of the VH CDR3 and the VL CDR3 of, (iii) at least 1, 2, 3, 4, 5, or 6 CDRs that are same as the corresponding CDR(s) of, or (iv) 6 CDRs that are all the same as the 6 CDRs of any one of the antibodies disclosed in Tables 3-6. In some embodiments, the antibody is VYD225, VYD224, VYD223, ADI-75696, ADI-75864, ADI-75620, ADI-75738, ADI-75700, ADI-75859, ADI-75684, ADI-75754, ADI-75648, ADI-75632, ADI-75741, ADI-75725, ADI-75717, ADI-75706, ADI-75699, ADI-75747, or ADI-75773. In some embodiments, the antibody is VYD224. In some embodiments, the antibody is VYD225.

In some embodiments, the disclosure contemplates anti-CoV-S antibodies or antigen-binding antibody fragments, wherein (a) the VH comprises an amino acid sequence with at least 80, 85, 90, 91, 92, 93, 94, 95, 96, 97, 98, 99, or 100% sequence identity to the amino acid sequence of the VH of, and (b) the VL comprises an amino acid sequence with at least 80, 85, 90, 91, 92, 93, 94, 95, 96, 97, 98, 99, or 100% sequence identity to the amino acid sequence of the VL of any one of the disclosed antibodies in Tables 3-6. In some embodiments, the antibody is VYD225, VYD224, VYD223, ADI-75696, ADI-75864, ADI-75620, ADI-75738, ADI-75700, ADI-75859, ADI-75684, ADI-75754, ADI-75648, ADI-75632, ADI-75741, ADI-75725, ADI-75717, ADI-75706, ADI-75699, ADI-75747, or ADI-75773. In some embodiments, the antibody is VYD224. In some embodiments, the antibody is VYD225.

In further embodiments, the disclosure contemplates anti-CoV-S antibodies or antigen-binding antibody fragments which optionally may be affinity-matured, comprising (i) a VH CDR that is same as the VH CDR3 of, (ii) a VH CDR3 and VL CDR3, both of which as same as both of the VH CDR3 and the VL CDR3 of, (iii) at least 1, 2, 3, 4, 5, or 6 CDRs that are same as the corresponding CDR(s) of, or (iv) 6 CDRs that are all the same as the 6 CDRs of any one of the antibodies disclosed in Tables 3-6. In some embodiments, the antibody is VYD225, VYD224, VYD223, ADI-75696, ADI-75864, ADI-75620, ADI-75738, ADI-75700, ADI-75859, ADI-75684, ADI-75754, ADI-75648, ADI-75632, ADI-75741, ADI-75725, ADI-75717, ADI-75706, ADI-75699, ADI-75747, or ADI-75773. In some embodiments, the antibody is VYD224. In some embodiments, the antibody is VYD225.

In further embodiments, the disclosure contemplates anti-CoV-S antibodies or antigen-binding antibody fragments which optionally may be affinity-matured, comprising one of the CDR requirements (i)-(iv) of the immediately above paragraph, further wherein (a) the VH comprises an amino acid sequence with at least 80, 85, 90, 91, 92, 93, 94, 95, 96, 97, 98, 99, or 100% sequence identity to the amino acid sequence of the VH of, and (b) the VL comprises an amino acid sequence with at least 80, 85, 90, 91, 92, 93, 94, 95, 96, 97, 98, 99, or 100% sequence identity to the amino acid sequence of the VL of any one of the disclosed antibodies in Tables 3-6. In some embodiments, the antibody is VYD225, VYD224, VYD223 ADI-75696, ADI-75864, ADI-75620, ADI-75738, ADI-75700, ADI-75859, ADI-75684, ADI-75754, ADI-75648, ADI-75632, ADI-75741, ADI-75725, ADI-75717, ADI-75706, ADI-75699, ADI-75747, or ADI-75773. In some embodiments, the antibody is VYD224. In some embodiments, the antibody is VYD225, or an antigen-binding portion thereof.

In other embodiments, the disclosure includes antibodies and antigen-binding fragments having binding specificity to COV-S, which optionally may be affinity-matured, that bind the same epitope as any one of antibodies disclosed in Tables 3-6. In some embodiments, the antibody is VYD225, VYD224, VYD223 ADI-75696, ADI-75864, ADI-75620, ADI-75738, ADI-75700, ADI-75859, ADI-75684, ADI-75754, ADI-75648, ADI-75632, ADI-75741, ADI-75725, ADI-75717, ADI-75706, ADI-75699, ADI-75747, or ADI-75773. In some embodiments, the antibody is VYD224. In some embodiments, the antibody is VYD225, or an antigen-binding portion thereof.

In other embodiments, the anti-CoV-S antibodies and antigen-binding fragments of the disclosure which optionally may be affinity-matured, comprise, or alternatively consist of, combinations of one or more of the FRs, CDRs, the VH and VL sequences, and the heavy chain and light chain sequences set forth above, including all of them, or sequences that are at least 90%, 91%, 92%, 93%, 94%, 95%, 96%, 97%, 98%, or 99% identical thereto.

In a further embodiment of the disclosure, antigen-binding fragments comprise, or alternatively consist of, Fab fragments having binding specificity for CoV-S. The Fab fragment preferably includes the VH and the VL sequence of antibodies in Tables 3-6, or sequences that are at least 90%, 91%, 92%, 93%, 94%, 95%, 96%, 97%, 98%, or 99% identical thereto. This embodiment of the disclosure further includes Fabs containing additions, deletions, and variants of such VH and VL sequence while retaining binding specificity for CoV-S.

In some embodiments of the disclosure described herein, Fab fragments may be produced by enzymatic digestion (e.g., papain) of the parent full antibody. In another embodiment of the disclosure, anti-CoV-S antibodies such as antibodies disclosed in Tables 3-6, and Fab fragments thereof may be produced via expression in mammalian cells, such as CHO, NSO, or HEK 293 cells, fungal, insect, or microbial systems, such as yeast cells.

In additional embodiments, the disclosure is further directed to polynucleotides encoding antibody polypeptides having binding specificity to COV-S, including the VH and VL of antibodies in Tables 3-6, as well as fragments, variants, optionally affinity-matured variants, and combinations of one or more of the FRs, CDRs, the VH and VL sequences, and the heavy chain and light chain sequences set forth above, including all of them, or sequences that are at least 90%, 91%, 92%, 93%, 94%, 95%, 96%, 97%, 98%, or 99% identical thereto. In some embodiments, the antibody is VYD225, VYD224, VYD223, ADI-75696, ADI-75864, ADI-75620, ADI-75738, ADI-75700, ADI-75859, ADI-75684, ADI-75754, ADI-75648, ADI-75632, ADI-75741, ADI-75725, ADI-75717, ADI-75706, ADI-75699, ADI-75747, or ADI-75773. In some embodiments, the antibody is VYD224. In some embodiments, the antibody is VYD225, or an antigen-binding portion thereof.

In other embodiments, the disclosure contemplates isolated anti-CoV-S antibodies and antigen binding fragments comprising (i) a VH which is same as the VH of one antibody selected from Tables 3 and 5; and (ii) a VL which is same as the VL of another antibody selected from Tables 4 and 6, or a variant thereof, wherein optionally one or more of the framework region residues (“FR residues”) and/or CDR residues in said VH or VL polypeptide has been substituted with another amino acid residue resulting in an anti-CoV-S antibody that binds, e.g., specifically binds, CoV-S. In some embodiments, the antibody is VYD225, VYD224, VYD223, ADI-75696, ADI-75864, ADI-75620, ADI-75738, ADI-75700, ADI-75859, ADI-75684, ADI-75754, ADI-75648, ADI-75632, ADI-75741, ADI-75725, ADI-75717, ADI-75706, ADI-75699, ADI-75747, or ADI-75773. In some embodiments, the antibody is VYD224. In some embodiments, the antibody is VYD225, or an antigen-binding portion thereof.

The disclosure also includes humanized, primatized and other chimeric forms of these antibodies. The chimeric and humanized antibodies may include an Fc derived from IgG1, IgG2, IgG3, or IgG4 constant regions.

In some embodiments of the disclosure, the chimeric or humanized antibodies or fragments or VH or VL polypeptides originate or are derived from one or more human antibodies, e.g., a human antibody identified from a clonal human B cell population.

In some aspects, the disclosure provides vectors comprising a nucleic acid molecule encoding an anti-CoV-S antibody or fragment thereof as disclosed herein. In some embodiments, the disclosure provides host cells comprising a nucleic acid molecule encoding an anti-CoV-S antibody or fragment thereof as disclosed herein.

In some aspects, the disclosure provides isolated antibodies or antigen binding fragments thereof that competes for binding to CoV-S with an antibody or antigen binding fragment thereof disclosed herein.

In some aspects, the disclosure provides a nucleic acid molecule encoding any of the antibodies or antigen binding fragments disclosed herein.

In some aspects, the disclosure provides a pharmaceutical or diagnostic composition comprising at least one antibody or antigen binding fragment thereof as disclosed herein.

In some aspects, the disclosure provides a method for treating or preventing a condition associated with elevated CoV-S levels in a subject, comprising administering to a subject in need thereof an effective amount of at least one isolated antibody or antigen binding fragment thereof as disclosed herein.

In some aspects, the disclosure provides a method of inhibiting binding of COV-S to its receptor (e.g., ACE2, L-SIGN, CD209L, DPP4, CD26) or an S protein-priming protein (e.g., TMPRSS2) in a subject comprising administering an effective amount of at least one antibody or antigen binding fragment thereof as disclosed herein. For example, administering one or more of the antibodies in Tables 3-6 may inhibit binding of CoV-S to its receptor, e.g., ACE2.

In some aspects, the disclosure provides an antibody or antigen binding fragment thereof that selectively binds to CoV-S, wherein the antibody or antigen binding fragment thereof binds to CoV-S with a K_{D} of less than or equal to 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, or 10⁻¹³ M; preferably, with a K_{D} of less than or equal to 5×10⁻¹⁰ M, 10⁻¹⁰ M, 5×10⁻¹¹ M, 10⁻¹¹ M, 5×10⁻¹² M, or 10⁻¹² M; more preferably, with a K_{D} that is less than about 100 pM, less than about 50 pM, less than about 40 pM, less than about 25 pM, less than about 1 pM, between about 10 pM and about 100 pM, between about 1 pM and about 100 pM, or between about 1 pM and about 10 pM. Preferably, the anti-CoV-S antibody or antigen binding fragment has cross-reactivity to the S protein of CoV other than SARS-COV-S or SARS-COV-2-S.

The inventive antibodies and antigen binding fragments thereof may be modified post-translationally to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

Antibodies and antigen binding fragments thereof may also be chemically modified to provide additional advantages such as increased solubility, stability and circulating time (in vivo half-life) of the polypeptide, or decreased immunogenicity (See U.S. Pat. No. 4,179,337). The chemical moieties for derivatization may be selected from water soluble polymers such as polyethylene glycol, ethylene glycol/propylene glycol copolymers, carboxymethylcellulose, dextran, polyvinyl alcohol, and the like. The antibodies and fragments thereof may be modified at random positions within the molecule, or at predetermined positions within the molecule and may include one, two, three, or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa. Branched polyethylene glycols are described, for example, in U.S. Pat. No. 5,643,575; Morpurgo et al., Appl. Biochem. Biotechnol., 56:59-72 (1996); Vorobjev et al., Nucleosides and Nucleotides, 18:2745-2750 (1999); and Caliceti et al., Bioconjug. Chem., 10:638-646 (1999), the disclosures of each of which are incorporated herein by reference.

There are a number of attachment methods available to those skilled in the art (See e.g., EP 0 401 384, herein incorporated by reference, disclosing a method of coupling PEG to G-CSF; and Malik et al., Exp. Hematol., 20:1028-1035 (1992) (reporting pegylation of GM-CSF using tresyl chloride)). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

As described above, polyethylene glycol may be attached to proteins via linkage to any of a number of amino acid residues. For example, polyethylene glycol can be linked to polypeptides via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) or to more than one type of amino acid residue (e.g., lysine, histidine, aspartic acid, glutamic acid, cysteine and combinations thereof).

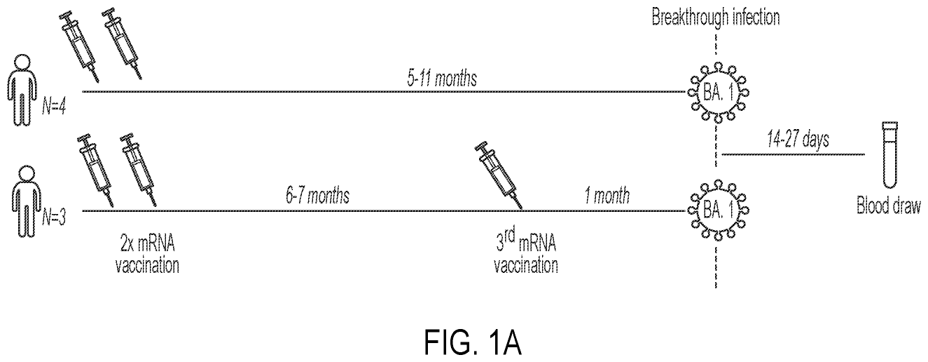

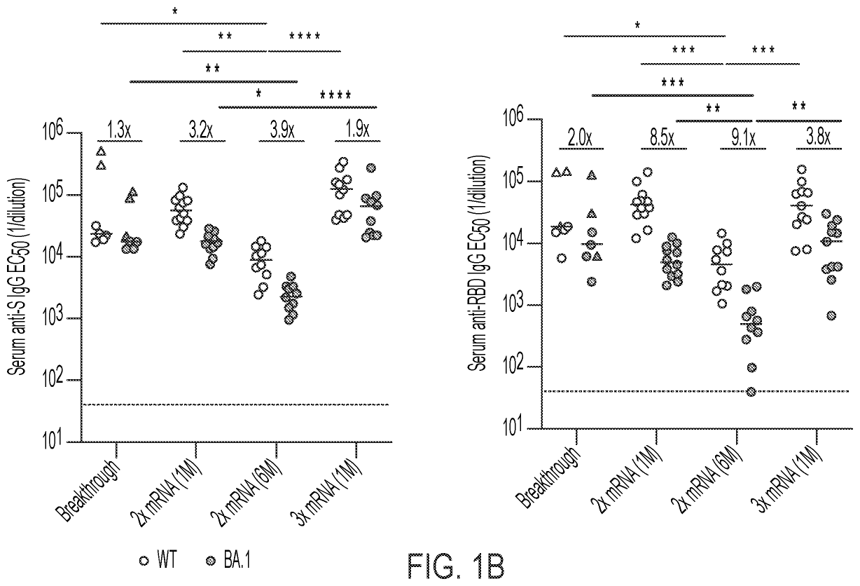

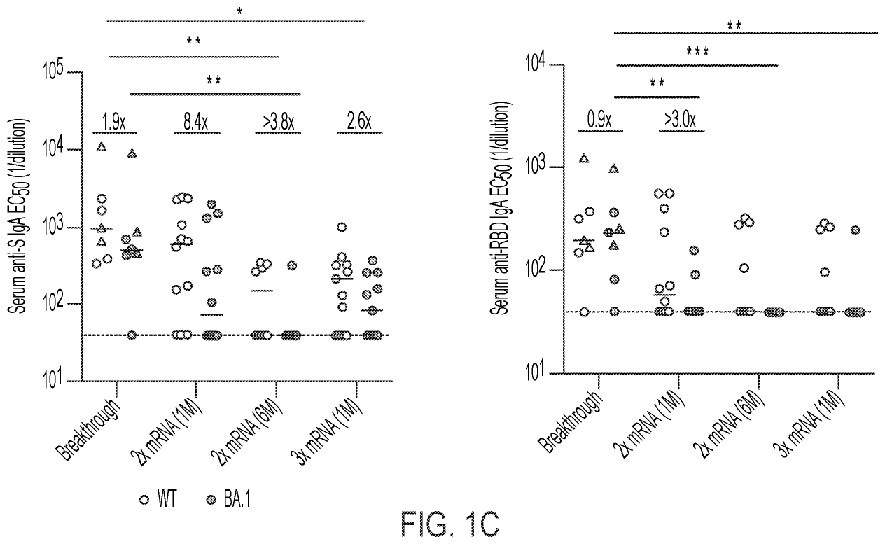

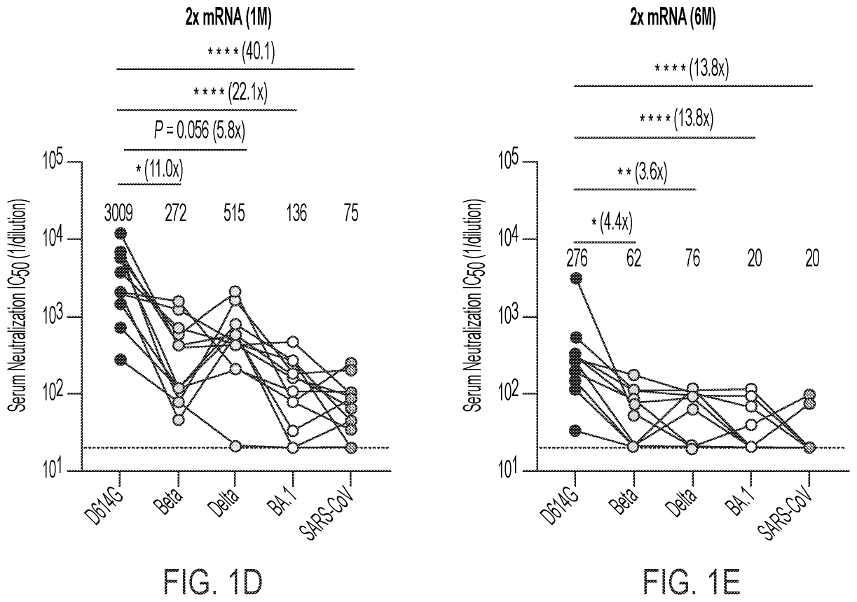

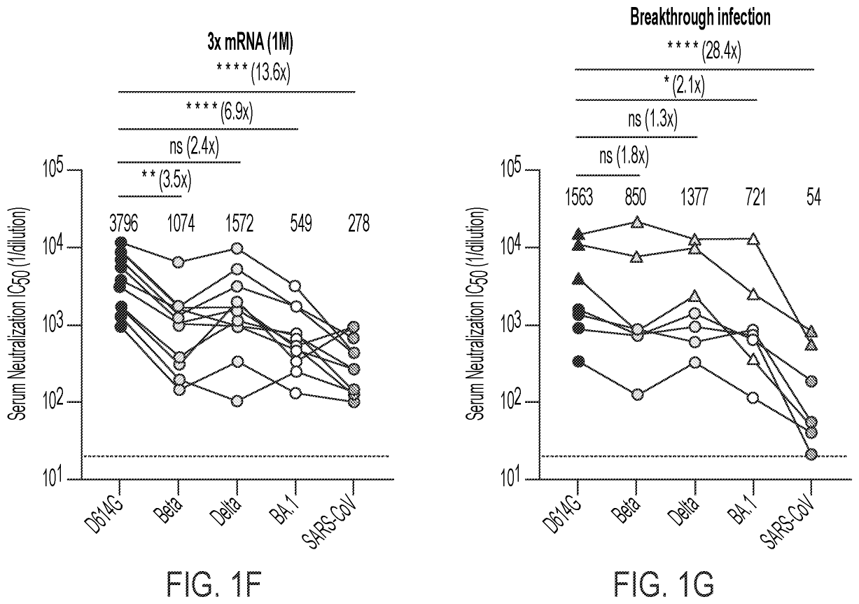

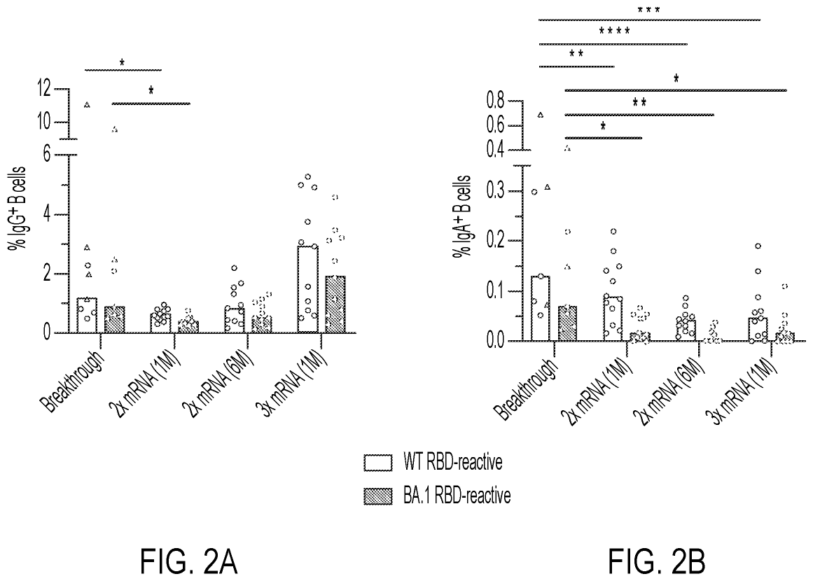

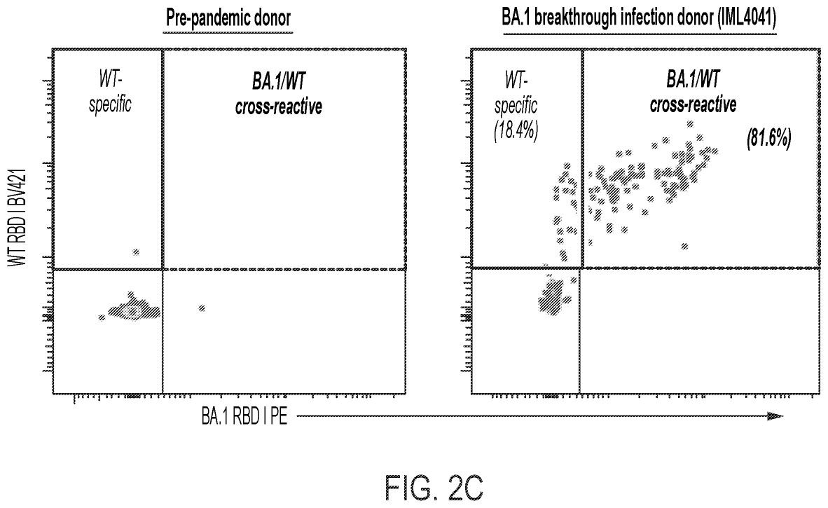

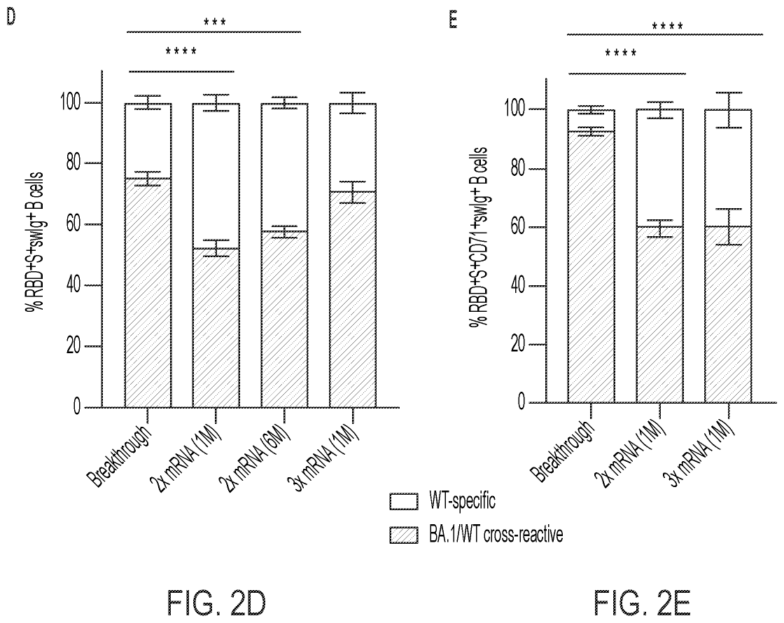

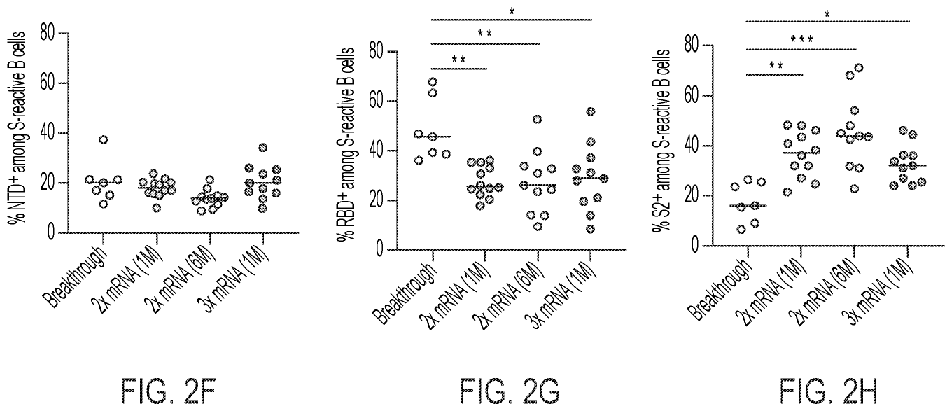

Alternatively, antibodies or antigen binding fragments thereof having increased in vivo half-lives may be produced via fusion with albumin (including but not limited to recombinant human serum albumin or fragments or variants thereof (See, e.g., U.S. Pat. No. 5,876,969, EP 0 413 622, and U.S. Pat. No. 5,766,883, herein incorporated by reference in their entirety)), or other circulating blood proteins such as transferrin or ferritin. In a preferred embodiment, polypeptides and/or antibodies of the present disclosure (including fragments or variants thereof) are fused with the mature form of human serum albumin (i.e., amino acids 1-585 of human serum albumin as shown in FIGS. 1 and 2 of EP 0 322 094) which is herein incorporated by reference in its entirety. Polynucleotides encoding fusion proteins of the disclosure are also encompassed by the disclosure.

Regarding detectable moieties, further exemplary enzymes include, but are not limited to, horseradish peroxidase, acetylcholinesterase, alkaline phosphatase, beta-galactosidase, and luciferase. Further exemplary fluorescent materials include, but are not limited to, rhodamine, fluorescein, fluorescein isothiocyanate, umbelliferone, dichlorotriazinylamine, phycoerythrin, and dansyl chloride. Further exemplary chemiluminescent moieties include, but are not limited to, luminol. Further exemplary bioluminescent materials include, but are not limited to, luciferin and aequorin. Further exemplary radioactive materials include, but are not limited to, Iodine 125 (¹²⁵I), Carbon 14 (¹⁴C), Sulfur 35 (³⁵S), Tritium (³H) and Phosphorus 32 (³²P).

Methods are known in the art for conjugating an antibody or antigen binding fragment thereof to a detectable moiety and the like, such as for example those methods described by Hunter et al., Nature, 144:945 (1962); David et al., Biochemistry, 13:1014 (1974); Pain et al., J. Immunol. Meth., 40:219 (1981); and Nygren, J., Histochem. and Cytochem., 30:407 (1982).

Embodiments described herein further include variants and equivalents that are substantially homologous to the antibodies, antibody fragments, diabodies, SMIPs, camelbodies, nanobodies, IgNAR, polypeptides, variable regions, and CDRs set forth herein. These may contain, e.g., conservative substitution mutations, (i.e., the substitution of one or more amino acids by similar amino acids). For example, conservative substitution refers to the substitution of an amino acid with another within the same general class, e.g., one acidic amino acid with another acidic amino acid, one basic amino acid with another basic amino acid, or one neutral amino acid by another neutral amino acid. The intent of a conservative amino acid substitution is well known in the art.

In other embodiments, the disclosure contemplates polypeptide sequences having at least 90% or greater sequence homology to any one or more of the polypeptide sequences of antigen binding fragments, variable regions and CDRs set forth herein. More preferably, the disclosure contemplates polypeptide sequences having at least 95% or greater sequence homology, even more preferably at least 98% or greater sequence homology, and still more preferably at least 99% or greater sequence homology to any one or more of the polypeptide sequences of antigen binding fragments, variable regions, and CDRs set forth herein.

Methods for determining homology between nucleic acid and amino acid sequences are well known to those of ordinary skill in the art.

In other embodiments, the disclosure further contemplates the above-recited polypeptide homologs of the antigen binding fragments, variable regions and CDRs set forth herein further having anti-CoV-S activity. Non-limiting examples of anti-CoV-S activity are set forth herein, e.g., ability to inhibit CoV-S binding to its receptor such as ACE2 or L-SIGN or an S protein-priming protein, thereby resulting in the reduced entry of CoV into cells.

In other embodiments, the disclosure further contemplates the generation and use of antibodies that bind any of the foregoing sequences, including, but not limited to, anti-idiotypic antibodies. In an exemplary embodiment, such an anti-idiotypic antibody could be administered to a subject who has received an anti-CoV-S antibody to modulate, reduce, or neutralize, the effect of the anti-CoV-S antibody. Such antibodies could also be useful for treatment of an autoimmune disease characterized by the presence of anti-CoV-S antibodies. A further exemplary use of such antibodies, e.g., anti-idiotypic antibodies, is for detection of the anti-CoV-S antibodies of the present disclosure, for example to monitor the levels of the anti-CoV-S antibodies present in a subject's blood or other bodily fluids. For example, in one embodiment, the disclosure provides a method of using the anti-idiotypic antibody to monitor the in vivo levels of said anti-CoV-S antibody or antigen binding fragment thereof in a subject or to neutralize said anti-CoV-S antibody in a subject being administered said anti-CoV-S antibody or antigen binding fragment thereof.

The present disclosure also contemplates anti-CoV-S antibodies comprising any of the polypeptide or polynucleotide sequences described herein substituted for any of the other polynucleotide sequences described herein. For example, without limitation thereto, the present disclosure contemplates antibodies comprising the combination of any of the VL and VH sequences described herein, and further contemplates antibodies resulting from substitution of any of the CDR sequences described herein for any of the other CDR sequences described herein.

Another embodiment of the disclosure contemplates these polynucleotides incorporated into an expression vector for expression in mammalian cells such as CHO, NSO, or HEK-293 cells, or in fungal, insect, or microbial systems such as yeast cells. In one embodiment of the disclosure described herein, Fab fragments can be produced by enzymatic digestion (e.g., papain) of any one of the antibodies in Tables 3-6; following expression of the full-length polynucleotides in a suitable host. In another embodiment, anti-CoV-S antibodies, such as anyone the antibodies in Tables 3-6, or Fab fragments thereof, can be produced via expression of the polynucleotides encoding the any one of the antibodies in Tables 3-6, in mammalian cells such as CHO, NSO, or HEK 293 cells, fungal, insect, or microbial systems such as yeast cells. In some embodiments, the antibody is VYD225, VYD224, VYD223 ADI-75696, ADI-75864, ADI-75620, ADI-75738, ADI-75700, ADI-75859, ADI-75684, ADI-75754, ADI-75648, ADI-75632, ADI-75741, ADI-75725, ADI-75717, ADI-75706, ADI-75699, ADI-75747, or ADI-75773, or an antigen-binding portion thereof. In some embodiments, the antibody is VYD224. In some embodiments, the antibody is VYD225, or an antigen-binding portion thereof.

Host cells and vectors comprising said polynucleotides are also contemplated.

The disclosure further contemplates vectors comprising the polynucleotide sequences encoding the variable heavy and light chain polypeptide sequences, as well as the individual CDRs (hypervariable regions), as set forth herein, as well as host cells comprising said vector sequences. In embodiments of the disclosure, the host cells are mammalian cells, such as CHO cells. In embodiments of the disclosure, the host cells are yeast cells.

### D. Antibody Combinations

The present disclosure also provides a composition comprising a combination of two or more antibodies, or antigen-binding fragment thereof, as disclosed herein.

In some embodiments, the two or more antibodies, or antigen-binding fragment thereof, within the composition, bind to the same epitope. In some embodiments, the two or more antibodies, or antigen-binding fragment thereof, within the composition, bind to different or non-overlapping epitopes.

In some embodiments, the composition comprises two or more isolated antibodies, or antigen-binding fragment thereof, selected from the group consisting of VYD225, VYD223 and VYD224.

In some embodiments, the composition comprises VYD225 and VYD224.

The composition comprising two or more antibodies or antigen-binding fragment thereof can be formulated into a pharmaceutical composition and be used for the methods as described herein.

### E. Antibody-Drug Conjugate Comprising Anti-CoV-S Antibody

In some aspects, the disclosure is further directed to antibody-drug conjugates (ADCs) comprising (a) any antibody or antigen-binding antibody fragment described herein; and (b) a drug conjugated to the antibody or antigen-binding antibody fragment, either directly or indirectly (e.g., via a linker), and the use of antibody-drug conjugates for the methods of the present application.

In some aspects, the drug may be, but not limited to, a cytotoxic drug, an apoptotic drug, an immunostimulatory drug, an anti-microbial drug, an antibacterial drug or vaccine, an antiviral drug, antihelminth drug, antiparasitic drug, an anti-inflammatory drug, antihistamine, an anti-fibrotic drug, an immunosuppressive drug, a steroid, a bronchodilator, a beta blocker, an ACE inhibitor, an enzyme, a serine protease inhibitor, a toxin, a radioisotope, a compound, a small molecule, a small molecule inhibitor, a protein, a peptide, a vector, a plasmid, a viral particle, a nanoparticle, a DNA molecule, an RNA molecule, an siRNA, an shRNA, a micro RNA, an oligonucleotide, and an imaging drug.

An antiviral drug may be remdesivir, favipiravir, darunavir, nelfinavir, saquinavir, lopinavir or ritonavir; an antihelminth drug may be ivermectin; an antiparasite drug may be hydroxychloroquine, chloroquine, or atovaquone; antibacterial drug or vaccine may be the tuberculosis vaccine BCG; an anti-inflammatory drug, may be ciclesonide, a TNF inhibitor (e.g., adalimumab), a TNF receptor inhibitor (e.g., etanercept), an IL-6 inhibitor (e.g., clazakizumab), an IL-6 receptor inhibitor (e.g., toclizumab), or metamizole; an antihistamine drug may be bepotastine; an ACE inhibitor may be moexipril; and a drug that inhibits priming of CoV-S may be a serine protease inhibitor such as nafamostat.

The toxin may be a bacterial, fungal, plant, or animal toxin, or a fragment thereof. Examples include, but are not limited to, diphtheria A chain, diphtheria toxin, exotoxin A chain, ricin A chain, abrin A chain, modeccin A chain, alpha sarcin, Aleurites fordii protein, a dianthin protein, or a Phytolacca americana protein.

The a cytotoxic drug or anti-proliferative drug may be, for example, but is not limited to, doxorubicin, daunorubicin, cucurbitacin, chaetocin, chaetoglobosin, chlamydocin, calicheamicin, nemorubicin, cryptophyscin, mensacarcin, ansamitocin, mitomycin C, geldanamycin, mechercharmycin, rebeccamycin, safracin, okilactomycin, oligomycin, actinomycin, sandramycin, hypothemycin, polyketomycin, hydroxyellipticine, thiocolchicine, methotrexate, triptolide, taltobulin, lactacystin, dolastatin, auristatin, monomethyl auristatin E (MMAE), monomethyl auristatin F (MMAF), telomestatin, tubastatin A, combretastatin, maytansinoid, MMAD, MMAF, DM1, DM4, DTT, 16-GMB-APA-GA, 17-DMAP-GA, JW 55, pyrrolobenzodiazepine, SN-38, Ro 5-3335, puwainaphycin, duocarmycin, bafilomycin, taxoid, tubulysin, ferulenol, lusiol A, fumagillin, hygrolidin, glucopiericidin, amanitin, ansatrienin, cinerubin, phallacidin, phalloidin, phytosphongosine, piericidin, poronetin, phodophyllotoxin, gramicidin A, sanguinarine, sinefungin, herboxidiene, microcolin B, microcystin, muscotoxin A, tolytoxin, tripolin A, myoseverin, mytoxin B, nocuolin A, psuedolaric acid B, pseurotin A, cyclopamine, curvulin, colchicine, aphidicolin, englerin, cordycepin, apoptolidin, epothilone A, limaquinone, isatropolone, isofistularin, quinaldopeptin, ixabepilone, aeroplysinin, arruginosin, agrochelin, epothilone, or a derivative thereof (for example, see Polakis P. et al., Pharmacol Rev. 2016 January; 68 (1): 3-19. doi: 10.1124/pr.114.009373) (the drugs may be obtained from many vendors, including Creative Biolabs®).

The radioisotope may be for example, but is not limited to, At²¹¹, I¹³¹, In¹³¹, I¹²⁵, Y⁹⁰, Re¹⁸⁶, Re¹⁸⁸, Sm¹⁵³, Bi²¹², P³², Pb²¹² and radioactive isotopes of Lu.

In certain embodiments, the drug may be, but is not limited to, MMAE or MMAF.

In some embodiments, the Ab or antigen-binding Ab fragment is directly conjugated to the drug to form an ADC.

In some embodiments, the antibody or antigen-binding antibody fragment is indirectly conjugated to the drug to form an ADC.

Any appropriate conjugation method may be used to generate an ADC (for example, Nolting B. Methods Mol Biol. 2013; 1045:71-100. doi: 10.1007/978-1-62703-541-5_5; Jain N. et al., Pharm Res. 2015 November; 32 (11): 3526-40. doi: 10.1007/s11095-015-1657-7. Epub 2015 Mar. 11; Tsuchikama K. et al., Protein Cell. 2018 January; 9 (1): 33-46. doi: 10.1007/s13238-016-0323-0. Epub 2016 Oct. 14; Polakis P. et al., Pharmacol Rev. 2016 January; 68 (1): 3-19. doi: 10.1124/pr.114.009373). Examples of methods that may be used to perform conjugation include, but are not limited to, chemical conjugation and enzymatic conjugation.

Chemical conjugation may utilize, for example, but is not limited to, lysine amide coupling, cysteine coupling, and/or non-natural amino acid incorporation by genetic engineering. Enzymatic conjugation may utilize, for example, but is not limited to, transpeptidation using sortase, transpeptidation using microbial transglutaminase, and/or N-Glycan engineering.

In certain aspects, one or more of cleavable linkers may be used for conjugation. The cleavable linker may enable cleavage of the drug upon responding to, for example, but not limited to, an environmental difference between the extracellular and intracellular environments (pH, redox potential, etc.) or by specific lysosomal enzymes.

Examples of the cleavable linker include, but are not limited to, hydrazone linkers, peptide linkers including cathepsin B-responsive linkers, such as valine-citrulline (vc) linker, disulfide linkers such as N-succinimidyl-4-(2-pyridyldithio) (SPP) linker or N-succinimidyl-4-(2-pyridyldithio) butanoate (SPDB) linker, and pyrophosphate diester linkers.

Alternatively or simultaneously, one or more of non-cleavable linkers may be used. Examples of non-cleavable linkers include thioether linkers, such as N-succinimidyl 4-(N-maleimidomethyl) cyclohexane-1-carboxylate (SMCC), and maleimidocaproyl (mc) linkers. Generally, non-cleavable linkers are more resistant to proteolytic degradation and more stable compared to cleavable linkers.

### F. Chimeric Antigen Receptor Comprising Anti-CoV-S Antigen-Binding Antibody Fragment

The present application further provides the use of chimeric antigen receptor comprising the anti-CoV-S antigen-binding fragment for the methods of the disclosure. In some embodiments, a compound specific to CoV-S according to the present disclosure may be a chimeric antigen receptor (CAR). In particular, the CARs of the present disclosure comprise an antigen binding (AB) domain that binds to CoV-S, a transmembrane (TM) domain, and an intracellular signaling (ICS) domain.

In some embodiments, a CAR may comprise a hinge that joins the AB domain and said TM domain.

In some embodiments, the CAR may comprise one or more costimulatory (CS) domains.

### AB Domain

A CAR according to the disclosure will comprise an antigen-binding (AB) domain which binds to COV-S. In some embodiments, the AB domain of the CAR may comprise any of the anti-COV-S antigen-binding antibody fragments disclosed herein.

In some embodiments, the AB domain of the CAR may comprise any of the antigen-binding domain of any of the anti-COV-S antibodies disclosed herein.

In some embodiments, the AB domain of the CAR may comprise any of the anti-COV-S antibodies, anti-COV-S antigen-binding antibody fragments, anti-COV-S multi-specific Abs, anti-COV-S multi-specific antigen-binding antibody fragments, and anti-COV-S ADCs disclosed herein, or the ABD thereof.

In some embodiments, the AB domain of the CAR may comprise an anti-COV-S scFv.

In some embodiments, the AB domain may comprise an amino acid sequence at least 80%, at least 85%, at least 90%, at least 91%, at least 92%, at least 93%, at least 94%, at least 95%, at least 96%, at least 97%, at least 98%, at least 99%, or 100% identical to an scFv comprising the VH and VL of an antibody disclosed in Tables 3-6.

In some aspects, the AB domain may compete for binding to COV-S with one or more antibodies disclosed in Tables 3-6.

### Hinge

In some embodiments, the CAR may comprise a hinge sequence between the AB domain and the TM domain. One of the ordinary skill in the art will appreciate that a hinge sequence is a short sequence of amino acids that facilitates flexibility (see, e.g. Woof J. M. et al., Nat. Rev. Immunol., 4 (2): 89-99 (2004)). The hinge sequence can be any suitable sequence derived or obtained from any suitable molecule.

In some embodiments, the length of the hinge sequence may be optimized based on the desired length of the extracellular portion of the CAR, which may be based on the location of the epitope within the target molecule. For example, if the epitope is in the membrane proximal region within the target molecule, longer hinges may be optimal.

In some embodiments, the hinge may be derived from or include at least a portion of an immunoglobulin Fc region, for example, an IgG1 Fc region, an IgG2 Fc region, an IgG3 Fc region, an IgG4 Fc region, an IgE Fc region, an IgM Fc region, or an IgA Fc region. In certain embodiments, the hinge includes at least a portion of an IgG1, an IgG2, an IgG3, an IgG4, an IgE, an IgM, or an IgA immunoglobulin Fc region that falls within its CH2 and CH3 domains. In some embodiments, the hinge may also include at least a portion of a corresponding immunoglobulin hinge region. In some embodiments, the hinge is derived from or includes at least a portion of a modified immunoglobulin Fc region, for example, a modified IgG1 Fc region, a modified IgG2 Fc region, a modified IgG3 Fc region, a modified IgG4 Fc region, a modified IgE Fc region, a modified IgM Fc region, or a modified IgA Fc region. The modified immunoglobulin Fc region may have one or more mutations (e.g., point mutations, insertions, deletions, duplications) resulting in one or more amino acid substitutions, modifications, or deletions that cause impaired binding of the hinge to an Fc receptor (FcR). In some aspects, the modified immunoglobulin Fc region may be designed with one or more mutations which result in one or more amino acid substitutions, modifications, or deletions that cause impaired binding of the hinge to one or more FcR including, but not limited to, FcγRI, FcγR2A, FcγR2B1, Fcγ2B2, Fcγ 3A, Fcγ 3B, FcεRI, FcεR2, FcαRI, Fcα/μR, or FcRn.

In some aspects, a portion of the immunoglobulin constant region may serve as a hinge between the AB domain, for example scFv or nanobody, and the TM domain. The hinge can be of a length that provides for increased responsiveness of the CAR-expressing cell following antigen binding, as compared to in the absence of the hinge. In some examples, the hinge is at or about 12 amino acids in length or is no more than 12 amino acids in length. Exemplary hinges include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a hinge has about 12 amino acids or less, about 119 amino acids or less, or about 229 amino acids or less. Exemplary hinges include a CD28 hinge, IgG4 hinge alone, IgG4 hinge linked to CH2 and CH3 domains, or IgG4 hinge linked to the CH3 domain. Exemplary hinges include, but are not limited to, those described in Hudecek M. et al. (2013) Clin. Cancer Res., 19:3153, international patent application publication number WO2014031687, U.S. Pat. No. 8,822,647 or published App. No. US2014/0271635.

Known hinge sequences include those derived from CD8 α molecule or a CD28 molecule.

### Transmembrane (TM) Domain

With respect to the TM domain, the CAR can be designed to comprise a TM domain that is fused to the AB domain of the CAR. A hinge sequence may be inserted between the AB domain and the TM domain. TM domains may be derived from a natural or from synthetic sources. Where the source is natural, the domain may be derived from any membrane-bound or transmembrane protein. Typically, a TM domain denotes a single transmembrane α helix of a transmembrane protein, also known as an integral protein. TM domains e.g., may be derived from (i.e. comprise at least the transmembrane region(s) of) CD28, CD3 ε, CD4, CD5, CD8, CD9, CD16, CD22, CD33, CD37, CD45, CD64, CD80, CD86, CD134, CD137, CD154, TCR α, TCR β, or CD3 zeta and/or TM domains containing functional variants thereof such as those retaining a substantial portion of the structural, e.g., transmembrane, properties thereof.

Alternatively, the TM domain may be synthetic, in which case the TM domain will comprise predominantly hydrophobic residues such as leucine and valine. Preferably a triplet of phenylalanine, tryptophan and valine will be found at each end of a synthetic TM domain. A TM domain is generally thermodynamically stable in a membrane. It may be a single a helix, a transmembrane β barrel, a β-helix of gramicidin A, or any other structure. Transmembrane helices are usually about 20 amino acids in length.

A well-used TM domain comprises the TM region of CD28, e.g., human CD28. Often, a short oligo- or polypeptide spacer, e.g., between 2 and 10 amino acids in length is used to form the linkage between the TM domain and the ICS domain(s) of the CAR.

### Intracellular Signaling (ICS) Domain and Costimulatory (CS) Domain

The ICS domain or the cytoplasmic domain of a CAR generally triggers or elicits activation of at least one of the normal effector functions of the cell in which the CAR has been placed. The term “effector function” refers to a specialized function of a cell. Effector function of a T cell, for example, may be cytolytic activity or helper activity including the secretion of cytokines. Thus, the term “intracellular signaling domain” or “ICS domain” refers to the portion of a protein which transduces the effector function signal and directs the cell to perform a specialized function. While usually the entire ICS domain can be employed, in many cases it is not necessary to use the entire chain. To the extent that a truncated portion of the intracellular signaling domain is used, such truncated portion may be used in place of the intact chain as long as it transduces the effector function signal. The term “intracellular signaling domain” or “ICS domain” is thus meant to include any truncated portion of the ICS domain sufficient to transduce the effector function signal.

Examples of known ICS domains include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

Signals generated through one ICS domain alone may be insufficient for full activation of a cell, and a secondary or costimulatory signal may also be required. In such cases, a costimulatory domain (CS domain) may be included in the cytoplasmic portion of a CAR. A CS domain is a domain that transduces such a secondary or costimulatory signal. In some instances, a CAR of the present disclosure may comprise two or more CS domains. The CS domain(s) may be placed upstream of the ICS domain or downstream of the ICS domain.

T cell activation can be mediated by two distinct classes of cytoplasmic signaling sequence: those that initiate antigen-dependent primary activation through the TCR (primary cytoplasmic signaling sequences) and those that act in an antigen-independent manner to provide a secondary or costimulatory signal (secondary cytoplasmic signaling sequences). Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. Such a cytoplasmic signaling sequence may be contained in the ICS or the CS domain of a CAR of the present disclosure.

Examples of ITAM-containing primary cytoplasmic signaling sequences include those derived from an ICS domain of a lymphocyte receptor chain, a TCR/CD3 complex protein, an Fc receptor subunit, an IL-2 receptor subunit, CD3ζ, FcR γ, FcRβ, CD3γ, CD3δ, CD3ε, CD5, CD22, CD66d, CD79a, CD79b, CD278 (ICOS), Fcε R1, DAP10, and DAP12. A well-used ICS domain comprises a cytoplasmic signaling sequence derived from CD3 zeta. In some instances, the CD3 ζ ICS domain may be combined with one or more of other cytoplasmic domain(s). For example, the cytoplasmic domain of the CAR can comprise a CD3 ζ ICS domain and a CS domain wherein a CS region refers to a portion of the CAR comprising the intracellular domain of a costimulatory molecule. A costimulatory molecule is a cell surface molecule other than an antigen receptor or their ligands that is required for an efficient response of lymphocytes to an antigen.

Examples of co-stimulatory molecules include an MHC class I molecule, TNF receptor proteins, Immunoglobulin-like proteins, cytokine receptors, integrins, signaling lymphocytic activation molecules (SLAM proteins), activating NK cell receptors, a Toll ligand receptor, B7-H3, BAFFR, BTLA, BLAME (SLAMF8), CD2, CD4, CD5, CD7, CD8 α, CD8 β, CD11a, LFA-1 (CD11a/CD18), CD11b, CD11c, CD11d, CD18, CD19, CD19a, CD27, CD28, CD29, CD30, CD40, CD49a, CD49D, CD49f, CD69, CD84, CD96 (Tactile), CD100 (SEMA4D), CD103, CRTAM, OX40 (CD134), 4-1BB (CD137), SLAM (SLAMF1, CD150, IPO-3), CD160 (BY55), SELPLG (CD162), DNAM1 (CD226), Ly9 (CD229), SLAMF4 (CD244, 2B4), ICOS (CD278), CEACAM1, CDS, CRTAM, DAP10, GADS, GITR, HVEM (LIGHTR), IA4, ICAM-1, IL2R β, IL2R γ, IL7R α, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGLI, SLAMF6 (NTB-A, Ly108), SLAMF7, SLP-76, TNFR2, TRANCE/RANKL, VLA1, VLA-6, a ligand that specifically binds with CD83, and the like. The ICS domain and the CS domain(s) of the CAR may be linked to each other in a random or specified order, optionally via a short oligo- or polypeptide linker, e.g., between 2 and 10 amino acids in length.

### Exemplary CAR Constructs

A CAR construct may comprise the following format: “AB domain-hinge-TM domain-CS domain-ICS domain.”

CARs of the present disclosure may comprise an amino acid sequence at least 80%, at least 85%, at least 90%, at least 91%, at least 92%, at least 93%, at least 94%, at least 95%, at least 96%, at least 97%, at least 98%, at least 99%, or 100% identical to any of the exemplary constructs below. In the exemplary constructs below, the “anti-CoV-S scFv” may be an scFv generated by linking the VH and VL (in the order of VH-linker-VL or VL-linker-VH) of any one of anti-CoV-S antibodies disclosed in Tables 3-6.

In some embodiments, a leader sequence (LS) may be placed upstream of the polynucleotide sequences encoding the CAR. The leader sequence facilitates expression of the CAR on the cell surface.

### Further Modification

CARs according to the present disclosure, nucleotide sequences encoding the same, vectors encoding the same, and cells comprising nucleotide sequences encoding said CARs may be further modified, engineered, optimized, or appended in order to provide or select for various features. These features may include, but are not limited to, efficacy, persistence, target specificity, reduced immunogenicity, multi-targeting, enhanced immune response, expansion, growth, reduced off-target effect, reduced subject toxicity, improved target cytotoxicity, improved attraction of disease alleviating immune cells, detection, selection, targeting, and the like. For example, the cells may be engineered to express another CAR, or to have a suicide mechanism, and may be modified to remove or modify expression of an endogenous receptor or molecule such as a TCR and/or MHC molecule.

In some embodiments, the vector or nucleic acid sequence encoding the CAR further encodes other genes. The vector or nucleic acid sequence may be constructed to allow for the co-expression of multiple genes using a multitude of techniques including co-transfection of two or more plasmids, the use of multiple or bidirectional promoters, or the creation of bicistronic or multicistronic vectors. The construction of multicistronic vectors may include the encoding of IRES elements or 2A peptides, such as T2A, P2A, E2A, or F2A (for example, see Kim, J. H., et al., “High cleavage efficiency of a 2A peptide derived from porcine teschovirus-1 in human cell lines, zebrafish and mice”, PLOS One. 2011; 6 (4)). The CAR expressing cell may further comprise a disruption to one or more endogenous genes.

### Efficacy

The CARs of the present disclosure and cells expressing these CARs may be further modified to improve efficacy against cells expressing the target molecule. The cells may be cells expressing COV-S. The cells expressing COV-S may be cancer cells, vascular cells, or any other target disease-associated cells. In some embodiments, the improved efficacy may be measured by increased cytotoxicity against cells expressing the target molecule, for example cytotoxicity against cancer cells. In some embodiments, the improved efficacy may also be measured by increased production of cytotoxic mediators such as, but not limited to, IFN γ, perforin, and granzyme B. In some embodiments, the improved efficacy may be shown by reduction in the signature cytokines of the diseases, or alleviated symptoms of the disease when the CAR expressing cells are administered to a subject. Other cytokines that may be reduced include TGF-beta, IL-6, IL-4, IL-10, and/or IL-13. the improved efficacy may be shown by COV-S-specific immune cell responses, such as T cell cytotoxicity. In case of cancer, improved efficacy may be shown by better tumor cytotoxicity, better infiltration into the tumor, reduction of immunosuppressive mediators, reduction in weight decrease, reduction in ascites, reduction in tumor burden, and/or increased lifespan. In case of autoimmune diseases, reduced responsiveness of autoreactive cells or decrease in autoreactive T cells, B cells, or Abs may represent improved efficacy. In some embodiments, gene expression profiles may be also investigated to evaluate the efficacy of the CAR.

In one aspect, the CAR expressing cells are further modified to evade or neutralize the activity of immunosuppressive mediators, including, but not limited to prostaglandin E2 (PGE2) and adenosine. In some embodiments, this evasion or neutralization is direct. In other embodiments, this evasion or neutralization is mediated via the inhibition of protein kinase A (PKA) with one or more binding partners, for example ezrin. In a specific embodiment, the CAR-expressing cells further express the peptide “regulatory subunit I anchoring disruptor” (RIAD). RIAD is thought to inhibit the association of protein kinase A (PKA) with ezrin, which thus prevents PKA's inhibition of TCR activation (Newick K. et al. Cancer Immunol Res. 2016 June; 4 (6): 541-51. doi: 10.1158/2326-6066.CIR-15-0263. Epub 2016 Apr. 4).

In some embodiments, the CAR expressing cells of the disclosure may induce a broad immune response, consistent with epitope spreading.

In some embodiments, the CAR expressing cells of the disclosure further comprise a homing mechanism. For example, the cell may transgenically express one or more stimulatory chemokines or cytokines or receptors thereof. In particular embodiments, the cells are genetically modified to express one or more stimulatory cytokines. In certain embodiments, one or more homing mechanisms are used to assist the inventive cells to accumulate more effectively to the disease site. In some embodiments, the CAR expressing cells are further modified to release inducible cytokines upon CAR activation, e.g., to attract or activate innate immune cells to a targeted cell (so-called fourth generation CARs or TRUCKS). In some embodiments, CARs may co-express homing molecules, e.g., CCR4 or CCR2b, to increase trafficking to the disease site.

### Controlling CAR Expression

In some instances, it may be advantageous to regulate the activity of the CAR or CAR expressing cells CAR. For example, inducing apoptosis using, e.g., a caspase fused to a dimerization domain (see, e.g., Di et al., N Engl. J. Med. 2011 Nov. 3; 365 (18): 1673-1683), can be used as a safety switch in the CAR therapy of the instant disclosure. In another example, CAR-expressing cells can also express an inducible Caspase-9 (iCaspase-9) molecule that, upon administration of a dimerizer drug (e.g., rimiducid (also called AP1903 (Bellicum Pharmaceuticals) or AP20187 (Ariad)) leads to activation of the Caspase-9 and apoptosis of the cells. The iCaspase-9 molecule contains a chemical inducer of dimerization (CID) binding domain that mediates dimerization in the presence of a CID. This results in inducible and selective depletion of CAR-expressing cells. In some cases, the iCaspase-9 molecule is encoded by a nucleic acid molecule separate from the CAR-encoding vector(s). In some cases, the iCaspase-9 molecule is encoded by the same nucleic acid molecule as the CAR-encoding vector. The iCaspase-9 can provide a safety switch to avoid any toxicity of CAR-expressing cells. See, e.g., Song et al. Cancer Gene Ther. 2008; 15 (10): 667-75; Clinical Trial Id. No. NCT02107963; and Di et al. N. Engl. J. Med. 2011; 365:1673-83.

Alternative strategies for regulating the CAR therapy of the instant disclosure include utilizing small molecules or antibodies that deactivate or turn off CAR activity, e.g., by deleting CAR-expressing cells, e.g., by inducing antibody dependent cell-mediated cytotoxicity (ADCC). For example, CAR-expressing cells described herein may also express an antigen that is recognized by molecules capable of inducing cell death, e.g., ADCC or compliment-induced cell death. For example, CAR expressing cells described herein may also express a receptor capable of being targeted by an antibody or antibody fragment. Examples of such receptors include EpCAM, VEGFR, integrins (e.g., integrins αvβ3, α4, αI3/4β3, α4β7, α5β1, αvβ3, αv), members of the TNF receptor superfamily (e.g., TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11a/LFA-1, CD15, CD18/ITGB2, CD19, CD20, CD22, CD23/IgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (e.g., versions preserving one or more extracellular epitopes but lacking one or more regions within the cytoplasmic domain). For example, CAR-expressing cells described herein may also express a truncated epidermal growth factor receptor (EGFR) which lacks signaling capacity but retains the epitope that is recognized by molecules capable of inducing ADCC, e.g., cetuximab (ERBITUX®), such that administration of cetuximab induces ADCC and subsequent depletion of the CAR-expressing cells (see, e.g., WO2011/056894, and Jonnalagadda et al., “Gene Ther. 2013; 20 (8) 853-860).

In some embodiments, the CAR cell comprises a polynucleotide encoding a suicide polypeptide, such as for example RQR8. See, e.g., WO2013153391A, which is hereby incorporated by reference in its entirety. In CAR cells comprising the polynucleotide, the suicide polypeptide may be expressed at the surface of a CAR cell. The suicide polypeptide may also comprise a signal peptide at the amino terminus. Another strategy includes expressing a highly compact marker/suicide gene that combines target epitopes from both CD32 and CD20 antigens in the CAR-expressing cells described herein, which binds rituximab, resulting in selective depletion of the CAR-expressing cells, e.g., by ADCC (see, e.g., Philip et al., Blood 2014; 124 (8) 1277-1287). Other methods for depleting CAR-expressing cells include administration of CAMPATH®, a monoclonal anti-CD52 antibody that selectively binds and targets mature lymphocytes, e.g., CAR-expressing cells, for destruction, e.g., by inducing ADCC. In other embodiments, the CAR-expressing cell can be selectively targeted using a CAR ligand, e.g., an anti-idiotypic antibody. In some embodiments, the anti-idiotypic antibody can cause effector cell activity, e.g., ADCC or ADC activities, thereby reducing the number of CAR-expressing cells. In other embodiments, the CAR ligand, e.g., the anti-idiotypic antibody, can be coupled to an agent that induces cell killing, e.g., a toxin, thereby reducing the number of CAR-expressing cells. Alternatively, the CAR molecules themselves can be configured such that the activity can be regulated, e.g., turned on and off, as described below.

In some embodiments, a regulatable CAR (RCAR) where the CAR activity can be controlled is desirable to optimize the safety and efficacy of a CAR therapy. In some embodiments, a RCAR comprises a set of polypeptides, typically two in the simplest embodiments, in which the components of a standard CAR described herein, e.g., an AB domain and an ICS domain, are partitioned on separate polypeptides or members. In some embodiments, the set of polypeptides include a dimerization switch that, upon the presence of a dimerization molecule, can couple the polypeptides to one another, e.g., can couple an AB domain to an ICS domain. Additional description and exemplary configurations of such regulatable CARs are provided herein and in International Publication No. WO 2015/090229, hereby incorporated by reference in its entirety.

In an aspect, an RCAR comprises two polypeptides or members: 1) an intracellular signaling member comprising an ICS domain, e.g., a primary ICS domain described herein, and a first switch domain; 2) an antigen binding member comprising an AB domain, e.g., that binds, e.g., specifically binds, a target molecule described herein, as described herein and a second switch domain. Optionally, the RCAR comprises a TM domain described herein. In an embodiment, a TM domain can be disposed on the intracellular signaling member, on the antigen binding member, or on both. Unless otherwise indicated, when members or elements of an RCAR are described herein, the order can be as provided, but other orders are included as well. In other words, in an embodiment, the order is as set out in the text, but in other embodiments, the order can be different. E.g., the order of elements on one side of a transmembrane region can be different from the example, e.g., the placement of a switch domain relative to an ICS domain can be different, e.g., reversed.

In some embodiments, the CAR expressing immune cell may only transiently express a CAR. For example, the cells of the disclosure may be transduced with mRNA comprising a nucleic acid sequence encoding an inventive CAR. In this vein, the present disclosure also includes an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequences (“UTRs”), a 5′ cap and/or Internal Ribosome Entry Site (IRES), the nucleic acid to be expressed, and a polyA tail, typically 50-2000 bases in length. RNA so produced can efficiently transfect different kinds of cells. In one embodiment, the template includes sequences for the CAR. In an embodiment, an RNA CAR vector is transduced into a cell by electroporation.

### Target Specificity

The CAR expressing cells of the present disclosure may further comprise one or more CARs, in addition to the first CAR. These additional CARs may or may not be specific for the target molecule of the first CAR. In some embodiments, the one or more additional CARs may act as inhibitory or activating CARs. In some aspects, the CAR of some embodiments is the stimulatory or activating CAR; in other aspects, it is the costimulatory CAR. In some embodiments, the cells further include inhibitory CARs (iCARs, see Fedorov et al., Sci. Transl. Medicine, 2013 December; 5 (215): 215ra172), such as a CAR recognizing an antigen other than the target molecule of the first CAR, whereby an activating signal delivered through the first CAR is diminished or inhibited by binding of the inhibitory CAR to its ligand, e.g., to reduce off-target effects.

In some embodiments, the AB domain of the CAR is or is part of an immunoconjugate, in which the AB domain is conjugated to one or more heterologous molecule(s), such as, but not limited to, a cytotoxic agent, an imaging agent, a detectable moiety, a multimerization domain, or other heterologous molecule. Cytotoxic agents include, but are not limited to, radioactive isotopes (e.g., At211, I131, I125, Y90, Re186, Re188, Sm153, Bi212, P32, Pb212 and radioactive isotopes of Lu); chemotherapeutic agents; growth inhibitory agents; enzymes and fragments thereof such as nucleolytic enzymes; antibiotics; toxins such as small molecule toxins or enzymatically active toxins. In some embodiments, the AB domain is conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In some embodiments, to enhance persistence, the cells of the disclosure may be further modified to overexpress pro-survival signals, reverse anti-survival signals, overexpress Bcl-xL, overexpress hTERT, lack Fas, or express a TGF-β dominant negative receptor. Persistence may also be facilitated by the administration of cytokines, e.g., IL-2, IL-7, and IL-15.

### G. B-Cell Screening and Isolation

In one embodiment, the present disclosure contemplates the preparation and isolation of a clonal population of antigen-specific B-cells that may be used for isolating at least one CoV-S antigen-specific cell, which can be used to produce a monoclonal antibody against CoV-S, which is specific to a desired CoV-S antigen, or a nucleic acid sequence corresponding to such an antibody. Methods of preparing and isolating said clonal population of antigen-specific B-cells are taught, for example, in U.S. Patent Publication No. US2007/0269868 to Carvalho-Jensen et al., the disclosure of which is herein incorporated by reference in its entirety. Methods of preparing and isolating said clonal population of antigen-specific B-cells are also taught herein in the examples. Methods of “enriching” a cell population by size or density are known in the art. See, e.g., U.S. Pat. No. 5,627,052. These steps can be used in addition to enriching the cell population by antigen-specificity.

### H. Methods of Producing Antibodies and Fragments Thereof

In another embodiment, the present disclosure contemplates methods for producing anti-CoV-S antibodies and fragments thereof. Methods of producing antibodies are well known to those of ordinary skill in the art. For example, methods of producing chimeric antibodies are now well known in the art (See, for example, U.S. Pat. No. 4,816,567 to Cabilly et al.; Morrison et al., Proc. Natl. Acad. Sci. U.S.A., 81:8651-55 (1984); Neuberger et al., Nature, 314:268-270 (1985); Boulianne, G. L. et al., Nature, 312:643-46 (1984), the disclosures of each of which are herein incorporated by reference in their entireties).

As mentioned above, methods of producing humanized antibodies are now well known in the art (See, for example, U.S. Pat. Nos. 5,530,101, 5,585,089, 5,693,762, and 6,180,370 to Queen et al; U.S. Pat. Nos. 5,225,539 and 6,548,640 to Winter; U.S. Pat. Nos. 6,054,297, 6,407,213 and 6,639,055 to Carter et al; U.S. Pat. No. 6,632,927 to Adair; Jones, P. T. et al., Nature, 321:522-525 (1986); Reichmann, L. et al., Nature, 332:323-327 (1988); Verhoeyen, M. et al., Science, 239:1534-36 (1988), the disclosures of each of which are herein incorporated by reference in their entireties).

Antibody polypeptides of the disclosure having CoV-S binding specificity may also be produced by constructing, using conventional techniques well known to those of ordinary skill in the art, an expression vector containing a promoter (optionally as a component of a eukaryotic or prokaryotic operon) and a DNA sequence encoding an antibody heavy chain in which the DNA sequence encoding the CDRs required for antibody specificity is derived from a non-human cell source, e.g., a rabbit or rodent B-cell source, while the DNA sequence encoding the remaining parts of the antibody chain is derived from a human cell source.

A second expression vector is produced using the same conventional means well known to those of ordinary skill in the art, said expression vector containing a promoter (optionally as a component of a eukaryotic or prokaryotic operon) and a DNA sequence encoding an antibody light chain in which the DNA sequence encoding the CDRs required for antibody specificity is derived from a non-human cell source, e.g., a rabbit or rodent B-cell source, while the DNA sequence encoding the remaining parts of the antibody chain is derived from a human cell source.

The expression vectors are transfected into a host cell by convention techniques well known to those of ordinary skill in the art to produce a transfected host cell, said transfected host cell cultured by conventional techniques well known to those of ordinary skill in the art to produce said antibody polypeptides.

The host cell may be co-transfected with the two expression vectors described above, the first expression vector containing DNA encoding a promoter (optionally as a component of a eukaryotic or prokaryotic operon) and a light chain-derived polypeptide and the second vector containing DNA encoding a promoter (optionally as a component of a eukaryotic or prokaryotic operon) and a heavy chain-derived polypeptide. The two vectors contain different selectable markers, but preferably achieve substantially equal expression of the heavy and light chain polypeptides. Alternatively, a single vector may be used, the vector including DNA encoding both the heavy and light chain polypeptides. The coding sequences for the heavy and light chains may comprise cDNA, genomic DNA, or both.

The host cells used to express the antibody polypeptides may be either a bacterial cell such as E. coli, or a eukaryotic cell such as P. pastoris. In one embodiment, a mammalian cell of a well-defined type for this purpose, such as a myeloma cell, a CHO cell line, a NSO cell line, or a HEK293 cell line may be used.

The general methods by which the vectors may be constructed, transfection methods required to produce the host cell and culturing methods required to produce the antibody polypeptides from said host cells all include conventional techniques. Although preferably the cell line used to produce the antibody is a mammalian cell line, any other suitable cell line, such as a bacterial cell line such as an E. coli-derived bacterial strain, or a yeast cell line, may alternatively be used.

Similarly, once produced the antibody polypeptides may be purified according to standard procedures in the art, such as for example cross-flow filtration, ammonium sulphate precipitation, affinity column chromatography, hydrophobic interaction chromatography (“HIC”), and the like.

The antibody polypeptides described herein may also be used for the design and synthesis of either peptide or non-peptide mimetics that would be useful for the same therapeutic applications as the antibody polypeptides of the disclosure (See, for example, Saragobi et al., Science, 253:792-795 (1991), the contents of which are herein incorporated by reference in its entirety).

In another embodiment, the present disclosure contemplates methods for humanizing antibody heavy and light chains which bind to CoV-S. Exemplary methods for humanizing antibody heavy and light chains that may be applied to anti-CoV-S antibodies are identified herein and are conventional in the art.

### I. Screening Assays

The screening assays described here may be used to identify high affinity anti-CoV-S Abs which may be useful in the treatment of diseases and disorders associated with CoV-S in subjects exhibiting symptoms of a CoV-S associated disease or disorder.

In some embodiments, the antibody is used as a diagnostic tool. The antibody can be used to assay the amount of CoV-S present in a sample and/or subject. As will be appreciated by one of skill in the art, such antibodies need not be neutralizing antibodies. In some embodiments, the diagnostic antibody is not a neutralizing antibody. In some embodiments, the diagnostic antibody binds to a different epitope than the neutralizing antibody binds to. In some embodiments, the two antibodies do not compete with one another.

In some embodiments, the antibodies disclosed herein are used or provided in an assay kit and/or method for the detection of CoV-S in mammalian tissues or cells in order to screen/diagnose for a disease or disorder associated with changes in levels of CoV-S. The kit comprises an antibody that binds CoV-S and means for indicating the binding of the antibody with CoV-S, if present, and optionally CoV-S protein levels. Various means for indicating the presence of an antibody can be used. For example, fluorophores, other molecular probes, or enzymes can be linked to the antibody and the presence of the antibody can be observed in a variety of ways. The method for screening for such disorders can involve the use of the kit, or simply the use of one of the disclosed antibodies and the determination of whether the antibody binds to CoV-S in a sample. As will be appreciated by one of skill in the art, high or elevated levels of CoV-S will result in larger amounts of the antibody binding to CoV-S in the sample. Thus, degree of antibody binding can be used to determine how much CoV-S is in a sample. Subjects or samples with an amount of CoV-S that is greater than a predetermined amount (e.g., an amount or range that a person without a CoV-S-related disorder would have) can be characterized as having a CoV-S-mediated disorder.

The present disclosure further provides for a kit for detecting binding of an anti-CoV-S antibody of the disclosure to CoV-S. In particular, the kit may be used to detect the presence of CoV-S specifically reactive with an anti-CoV-S antibody of the disclosure or an immunoreactive fragment thereof. The kit may also include an antibody bound to a substrate, a secondary antibody reactive with the antigen and a reagent for detecting a reaction of the secondary antibody with the antigen. Such a kit may be an ELISA kit and can comprise the substrate, primary and secondary antibodies when appropriate, and any other necessary reagents such as detectable moieties, enzyme substrates, and color reagents, for example as described herein. The diagnostic kit may also be in the form of an immunoblot kit. The diagnostic kit may also be in the form of a chemiluminescent kit (Meso Scale Discovery, Gaithersburg, MD). The diagnostic kit may also be a lanthanide-based detection kit (PerkinElmer, San Jose, CA).

A skilled clinician would understand that a biological sample includes, but is not limited to, sera, plasma, urine, fecal sample, saliva, mucous, pleural fluid, synovial fluid, and spinal fluid.

### J. Methods of Ameliorating or Reducing Symptoms of, or Treating, or Preventing, Diseases and Disorders Associated with CoV-S

The present disclosure provides methods for ameliorating or reducing the symptoms of, or treating, or preventing, diseases and disorders associated with CoV-S. The methods comprise administering an antibody, or antigen-binding fragment thereof, that displays broad activity against all SARS-COV-2 variants of concern (including the Omicron/BA.1 variant) as well as SARS-COV.

In some embodiments, an antibody or antigen-binding fragment thereof is capable of binding to the spike protein of a corona virus (CoV-S). In some embodiments, the CoV-S is the spike protein of SARS-COV (“SARS-COV-S”) and/or the spike protein of SARS-COV-2 (“SARS-COV-2-S”).

In certain embodiments, an antibody or antigen-binding fragment thereof is capable of binding to a SARS-COV-2 variant. In some embodiments, the SARS-COV-2-S is a B.1.1.7 variant, a B. 1.351 variant, a B.1.1.28 variant, a B. 1.429 variant, a P.1 variant, a B.1.617 variant (e.g., B.1.617.1 and B.1.617.2), a C.37 variant, a 1.621 variant, a AY.1 variant, a 1.623 variant, a C.36 variant, a A.27 variant, a AV.1 variant, a B.1.1.482 variant, a B.1.1.523 variant, a B.1.427 variant, a AY.4 variant, a AY.11 variant, variant, a D614G variant, or a B.1.1.529/BA.1 variant (also known as the Omicron variant) and its sublineages (e.g., BA1.1, BA.2, BA.2.75, BA.4, BA.5, BA.4.6, BQ.1, BQ.1.1, XBB, XBB.1, XBB.1.5, BJ.1, BM.1.1.1, BA.2.3.20, BF.7, XBC, BN.1, or CH.1.1).

In some embodiments, the antibody or antigen-binding fragment thereof binds to the receptor binding domain (RBD) of CoV-S, e.g., the RBD of CoV-s from the B.1.1.529/BA.1 variant, the BF.7 variant, the BQ.1.1 variant, the BA.2.75 variant, the XBB.1 variant, the BA.2 variant, the B. 1.351 variant, the B.1.617 variant, or the D614G variant.

Anti-CoV-S antibodies described in Tables 3-6, or antigen-binding fragments thereof, e.g., VYD225, as well as combinations, can also be administered in a therapeutically effective amount to patients in need of treatment of diseases and disorders associated with CoV-S in the form of a pharmaceutical composition as described in greater detail below.

Symptoms of CoV infection may include fever, cough, runny nose, congestion, sore throat, bronchitis, pneumonia, shortness of breath, chest pain, headache, muscle ache, chills, fatigue, conjunctivitis, diarrhea, loss of smell, and loss of taste. Complications and/or diseases/disorders associated with coronavirus infection may include, for example, bronchitis, pneumonia, respiratory failure, acute respiratory failure, organ failure, multi-organ system failure, pediatric inflammatory multisystem syndrome, acute respiratory distress syndrome (a severe lung condition that causes low oxygen in the blood and organs), blood clots, cardiac conditions, myocardial injury, myocarditis, heart failure, cardiac arrest, acute myocardial infarction, dysrhythmias, venous thromboembolism, post-intensive care syndrome, shock, anaphylactic shock, cytokine release syndrome, septic shock, disseminated intravascular coagulation, ischemic stroke, intracerebral hemorrhage, microangiopathic thrombosis, psychosis, seizure, nonconvulsive status epilepticus, traumatic brain injury, stroke, anoxic brain injury, encephalitis, posterior reversible leukoencephalopathy, necrotizing encephalopathy, post-infectious encephalitis, autoimmune mediated encephalitis, acute disseminated encephalomyelitis, acute kidney injury, acute liver injury, pancreatic injury, immune thrombocytopenia, subacute thyroiditis, gastrointestinal complications, aspergillosis, increased susceptibility to infection with another virus or bacteria, and/or pregnancy-related complications. Certain diseases and conditions, such as high blood pressure, type 1 diabetes, liver disease, overweight, chronic lung diseases including cystic fibrosis, pulmonary fibrosis, and asthma, compromised immune system due to transplant, use of an immunosuppressant, or HIV infection, and brain and nervous system condition, may increase the risk of CoV infection-associated complications and diseases.

Also, the subject anti-CoV-S antibodies and antigen-binding fragments may be used alone or in conjunction with other active agents, e.g., opioids and non-opioid analgesics such as NSAIDs to elicit analgesia. In some embodiments, aspirin and/or acetaminophen may be taken in conjunction with the subject anti-CoV-S antibody or antigen-binding fragment. Aspirin is another type of non-steroidal anti-inflammatory compound.

The subject antibodies potentially optionally may be combined with one or more of the following: (i) an antiviral drug, optionally, remdesivir, favipiravir, darunavir, nelfinavir, saquinavir, lopinavir, or ritonavir; (ii) an antihelminth drug, optionally ivermectin; (iii) an antiparasitic drug, optionally hydroxychloroquine, chloroquine, or atovaquone; (iv) antibacterial vaccine, optionally the tuberculosis vaccine BCG; or (v) an anti-inflammatory drug, optionally a steroid such as ciclesonide, a TNF inhibitor (e.g., adalimumab), a TNF receptor inhibitor (e.g., etanercept), an IL-6 inhibitor (e.g., clazakizumab), an IL-6 receptor inhibitor (e.g., toclizumab), or metamizole; (vi) an antihistamine drug, optionally bepotastine; (vii) an ACE inhibitor, which is optionally moexipril; or (viii) a drug that inhibits priming of CoV-S, optionally a serine protease inhibitor, further optionally nafamostat. in order to increase or enhance pain management. This may allow for such analgesic compounds to be administered for longer duration or at reduced dosages thereby potentially alleviating adverse side effects associated therewith.

In some embodiments, the anti-CoV-S antibodies and antigen-binding fragments disclosed herein are administered in combination with one or more antibodies as described in U.S. Provisional Application No. 63/143,456, filed on Jan. 29, 2021, the entire contents of which have been incorporated herein by reference. In some embodiments, the antibody to be administered in combination with the anti-CoV-S antibodies and antigen-binding fragments disclosed herein is ADI-58125, as described in U.S. Provisional Application No. 63/143,456. In one embodiment, the antibodies are administered at the same time. In another embodiment, the antibodies are administered sequentially.

The subject to which the pharmaceutical formulation is administered can be, e.g., any human or non-human animal needing such treatment, prevention and/or amelioration, or who would otherwise benefit from the inhibition or attenuation of CoV-S-mediated activity. For example, the subject can be an individual that is diagnosed with, or who is deemed to be at risk of being afflicted by any of the aforementioned diseases or disorders. In some instances the subject may be in an advanced state of CoV infection, e.g., a subject who is on a ventilator. In some instances, the subject can be one having one or more risk factors (such as advanced age, obesity, diabetes, etc, and others previously identified) which correlate to a poor CoV treatment or recovery prognosis. The present disclosure further includes the use of any of the pharmaceutical formulations disclosed herein in the manufacture of a medicament for the treatment, prevention and/or amelioration of any disease or disorder associated with CoV or CoV-S activity (including any of the above-mentioned exemplary diseases, disorders and conditions).

### K. Administration

In one embodiment, the anti-CoV-S antibodies described herein, or CoV-S binding fragments thereof, as well as combinations of said antibodies or antigen-binding fragments thereof, are administered to a subject at a concentration of between 0.1 mg/ml and about any one of 0.5, 1, 5, 10, 15 20, 25, 30, 35, 40, 45, 50, 55, 60, 65, 70, 75, 80, 85, 90, 95, 100, 110, 120, 130, 140, 150, 160, 170, 180, 190, or 200 mg/ml, +/−10% error.

In another embodiment, the anti-CoV-S antibodies and fragments thereof described herein are administered to a subject at a dose of between about 0.01 and 100.0 or 200.0 mg/kg of body weight of the recipient subject. In certain embodiments, depending on the type and severity of the CoV-S-related disease, about 1 μg/kg to 50 mg/kg (e.g., 0.1-20 mg/kg) of antibody is an initial candidate dosage for administration to the patient, whether, for example, by one or more separate administrations, or by continuous infusion. In another embodiment, about 1 μg/kg to 15 mg/kg (e.g., 0.1 mg/kg-10 mg/kg) of antibody is an initial candidate dosage for administration to the patient. A typical daily dosage might range from about 1 μg/kg to 100 mg/kg or more, depending on several factors, e.g., the particular mammal being treated, the clinical condition of the individual patient, the cause of the disorder, the site of delivery of the agent, the method of administration, the scheduling of administration, and other factors known to medical practitioners. However, other dosage regimens may be useful.

For example, in addition to the relative dosages (mg/kg) discussed herein, the subject anti-CoV-S antibodies and antigen-binding fragments thereof can be administered to a subject at an absolute dose (mg). Accordingly, in one embodiment, the anti-CoV-S antibodies and antigen-binding fragments thereof described herein are administered to a subject at a dose of between about 1 microgram and about 2000 milligrams regardless of the route of administration.

In some embodiments, the antibody, or antigen-binding fragment thereof, or the combination of said antibodies or antigen-binding fragments thereof, is administered at a dose of about 100 mg to about 5000 mg, about 100 mg to 4500 mg, about 100 mg to 4000 mg, about 100 mg to about 3500 mg, about 100 mg to about 3000 mg, about 100 mg to about 2500 mg, about 100 mg to about 2000 mg, about 200 mg to about 1500 mg, about 300 mg to about 600 mg, about 500 mg to about 1200 mg, about 300 mg to about 1200 mg, about 500 to about 1000 mg, about 1000 mg to about 1500 mg, about 1500 mg to about 2000 mg, about 2000 mg to about 2500 mg, about 2500 mg to about 3000 mg, about 3000 mg to about 3500 mg, about 3500 mg to about 4000 mg, about 4000 to about 4500 mg, or about 4500 mg to about 5000 mg.

In some embodiments, the antibody, or antigen-binding fragment thereof, or the combination of said antibodies or antigen-binding fragments thereof, is administered intramuscularly. In some embodiments, the antibody, or antigen-binding fragment thereof, or the combination of said antibodies or antigen-binding fragments thereof, is administered intravenously. In one embodiment, the antibody, or antigen-binding fragment thereof, or the combination of said antibodies and antigen-binding fragments thereof, is administered via an IV push. In another embodiment, the antibody, or antigen-binding fragment thereof, or the combination of said antibodies and antigen-binding fragments thereof, is administered via an IV bolus.

In some embodiments, the antibody, or antigen-binding fragment thereof, or the combination of said antibodies or antigen-binding fragments thereof, is administered intramuscularly at a dose of about 500 mg. In some embodiments, the antibody, or antigen-binding fragment thereof, or the combination of said antibodies or antigen-binding fragments thereof, is administered intramuscularly at a dose of about 600 mg. In some embodiments, the antibody, or antigen-binding fragment thereof, or the combination of said antibodies or antigen-binding fragments thereof, is administered intravenously at a dose of about 1200 mg. In some embodiments, the antibody, or antigen-binding fragment thereof, or the combination of said antibodies or antigen-binding fragments thereof, is administered intravenously at a dose of about 1500 mg. In some embodiments, the antibody, or antigen-binding fragment thereof, or the combination of said antibodies or antigen-binding fragments thereof, is administered intravenously at a dose of about 2000 mg. In some embodiments, the antibody, or antigen-binding fragment thereof, or the combination of said antibodies or antigen-binding fragments thereof, is administered intravenously at a dose of about 2500 mg. In some embodiments, the antibody, or antigen-binding fragment thereof, or the combination of said antibodies or antigen-binding fragments thereof, is administered intravenously at a dose of about 3000 mg. In some embodiments, the antibody, or antigen-binding fragment thereof, or the combination of said antibodies or antigen-binding fragments thereof, is administered intravenously at a dose of about 3500 mg. In some embodiments, the antibody, or antigen-binding fragment thereof, or the combination of said antibodies or antigen-binding fragments thereof, is administered intravenously at a dose of about 4000 mg. In some embodiments, the antibody, or antigen-binding fragment thereof, or the combination of said antibodies or antigen-binding fragments thereof, is administered intravenously at a dose of about 4500 mg. In some embodiments, the antibody, or antigen-binding fragment thereof, or the combination of said antibodies or antigen-binding fragments thereof, is administered intravenously at a dose of about 5000 mg.

In one embodiment, the antibody, or antigen-binding fragment thereof, or the combination of said antibodies or antigen-binding fragments thereof, is administered once. In one embodiment, the antibody, or antigen-binding fragment thereof, or the combination of said antibodies or antigen-binding fragments thereof, is administered weekly. In another embodiment, the antibody, or antigen-binding fragment thereof, or the combination of said antibodies or antigen-binding fragments thereof, is administered daily, weekly, every two weeks, monthly, every two months, or every three months. In one embodiment, the antibody, or antigen-binding fragment thereof, or the combination of said antibodies or antigen-binding fragments thereof, is administered weekly for about four weeks, once weekly for about a month, weekly for about 5 weeks, weekly for about 6 weeks, weekly for about 7 weeks, or weekly for about two months.

In another embodiment, the anti-CoV-S antibodies described herein, or anti-CoV-S antigen-binding fragments thereof, as well as combinations of said antibodies or antigen-binding fragments thereof, are administered to a recipient subject with a frequency of once every twenty-six weeks or less, such as once every sixteen weeks or less, once every eight weeks or less, once every four weeks or less, once every two weeks or less, once every week or less, or once daily or less.

According to preferred embodiments, the antibody containing medicament or pharmaceutical composition is peripherally administered to a subject via a route selected from one or more of: orally, sublingually, buccally, topically, rectally, via inhalation, transdermally, subcutaneously, intravenously, intra-arterially, or intramuscularly, via intracardiac administration, intraosseously, intradermally, intraperitoneally, transmucosally, vaginally, intravitreally, epicutaneously, intra-articularly, peri-articularly, or locally.

Fab fragments may be administered every two weeks or less, every week or less, once daily or less, multiple times per day, and/or every few hours. In one embodiment, a patient receives Fab fragments of 0.1 mg/kg to 40 mg/kg per day given in divided doses of 1 to 6 times a day, or in a continuous perfusion form, effective to obtain desired results.

It is to be understood that the concentration of the antibody or Fab administered to a given patient may be greater or lower than the exemplary administration concentrations set forth above.

A person of skill in the art would be able to determine an effective dosage and frequency of administration through routine experimentation, for example guided by the disclosure herein and the teachings in, Goodman & Gilman's The Pharmacological Basis of Therapeutics, Brunton, L. L. et al. editors, 11^{th} edition, New York, New York: McGraw-Hill (2006); Howland, R. D. et al., Pharmacology, Volume 864, Lippincott's illustrated reviews., Philadelphia, PA: Lippincott Williams & Wilkins (2006); and Golan, D. E., Principles of pharmacology: the pathophysiologic basis of drug therapy, Philadelphia, PA: Lippincott Williams & Wilkins (2007).

In another embodiment, the anti-CoV-S antibodies described herein, or CoV-S binding fragments thereof, as well as combinations of said antibodies or antigen-binding fragments thereof, are administered to a subject in a pharmaceutical formulation. In a preferred embodiment, the subject is a human.

A “pharmaceutical composition” or “medicament” refers to a chemical or biological composition suitable for administration to a subject, preferably a mammal, more preferably a human. Such compositions may be specifically formulated for administration via one or more of a number of routes, including but not limited to buccal, epicutaneous, epidural, inhalation, intraarterial, intracardial, intracerebroventricular, intradermal, intramuscular, intranasal, intraocular, intraperitoneal, intraspinal, intrathecal, intravenous, oral, parenteral, rectally via an enema or suppository, subcutaneous, subdermal, sublingual, transdermal, and transmucosal. In addition, administration can occur by means of injection, powder, liquid, gel, drops, or other means of administration.

In one embodiment, the anti-CoV-S antibodies described herein, or CoV-S binding fragments thereof, as well as combinations of said antibodies or antigen-binding fragments thereof, may be optionally administered in combination with one or more active agents. Such active agents include (i) an antiviral drug, optionally, remdesivir, favipiravir, darunavir, nelfinavir, saquinavir, lopinavir, or ritonavir; (ii) an antihelminth drug, optionally ivermectin; (iii) an antiparasitic drug, optionally hydroxychloroquine, chloroquine, or atovaquone; (iv) antibacterial vaccine, optionally the tuberculosis vaccine BCG; or (v) an anti-inflammatory drug, optionally a steroid such as ciclesonide, a TNF inhibitor (e.g., adalimumab), a TNF receptor inhibitor (e.g., etanercept), an IL-6 inhibitor (e.g., clazakizumab), an IL-6 receptor inhibitor (e.g., toclizumab), or metamizole; (vi) an antihistamine drug, optionally bepotastine; (vii) an ACE inhibitor, optionally moexipril; or (viii) a drug that inhibits priming of CoV-S, optionally a serine protease inhibitor, further optionally nafamostat.

An anti-histamine can be any compound that opposes the action of histamine or its release from cells (e.g., mast cells). Anti-histamines include but are not limited to acrivastine, astemizole, azatadine, azelastine, betatastine, brompheniramine, buclizine, cetirizine, cetirizine analogues, chlorpheniramine, clemastine, CS 560, cyproheptadine, desloratadine, dexchlorpheniramine, ebastine, epinastine, fexofenadine, HSR 609, hydroxyzine, levocabastine, loratadine, methscopolamine, mizolastine, norastemizole, phenindamine, promethazine, pyrilamine, terfenadine, and tranilast.

In CoV infection, respiratory symptoms are often exacerbated by additional bacterial infection. Therefore, such active agents may also be antibiotics, which include but are not limited to amikacin, aminoglycosides, amoxicillin, ampicillin, ansamycins, arsphenamine, azithromycin, azlocillin, aztreonam, bacitracin, carbacephem, carbapenems, carbenicillin, cefaclor, cefadroxil, cefalexin, cefalothin, cefalotin, cefamandole, cefazolin, cefdinir, cefditoren, cefepime, cefixime, cefoperazone, cefotaxime, cefoxitin, cefpodoxime, cefprozil, ceftazidime, ceftibuten, ceftizoxime, ceftobiprole, ceftriaxone, cefuroxime, cephalosporins, chloramphenicol, cilastatin, ciprofloxacin, clarithromycin, clindamycin, cloxacillin, colistin, co-trimoxazole, dalfopristin, demeclocycline, dicloxacillin, dirithromycin, doripenem, doxycycline, enoxacin, ertapenem, erythromycin, ethambutol, flucloxacillin, fosfomycin, furazolidone, fusidic acid, gatifloxacin, geldanamycin, gentamicin, glycopeptides, herbimycin, imipenem, isoniazid, kanamycin, levofloxacin, lincomycin, linezolid, lomefloxacin, loracarbef, macrolides, mafenide, meropenem, methicillin, metronidazole, mezlocillin, minocycline, monobactams, moxifloxacin, mupirocin, nafcillin, neomycin, netilmicin, nitrofurantoin, norfloxacin, ofloxacin, oxacillin, oxytetracycline, paromomycin, penicillin, penicillins, piperacillin, platensimycin, polymyxin B, polypeptides, prontosil, pyrazinamide, quinolones, quinupristin, rifampicin, rifampin, roxithromycin, spectinomycin, streptomycin, sulfacetamide, sulfamethizole, sulfanilamide, sulfasalazine, sulfisoxazole, sulfonamides, teicoplanin, telithromycin, tetracycline, tetracyclines, ticarcillin, tinidazole, tobramycin, trimethoprim, trimethoprim-sulfamethoxazole, troleandomycin, trovafloxacin, and vancomycin.

Active agents also include aldosterone, beclomethasone, betamethasone, corticosteroids, cortisol, cortisone acetate, deoxycorticosterone acetate, dexamethasone, fludrocortisone acetate, glucocorticoids, hydrocortisone, methylprednisolone, prednisolone, prednisone, steroids, and triamcinolone. Any suitable combination of these active agents is also contemplated.

A “pharmaceutical excipient” or a “pharmaceutically acceptable excipient” is a carrier, usually a liquid, in which an active therapeutic agent is formulated. In one embodiment, the active therapeutic agent is a humanized antibody described herein, or one or more fragments thereof. The excipient generally does not provide any pharmacological activity to the formulation, though it may provide chemical and/or biological stability, and release characteristics. Exemplary formulations can be found, for example, in Remington's Pharmaceutical Sciences, Gennaro, A. editor, 19^{th} edition, Philadelphia, PA: Williams and Wilkins (1995), which is incorporated by reference.

As used herein “pharmaceutically acceptable carrier” or “excipient” includes any and all solvents, dispersion media, coatings, antibacterial and antifungal agents, isotonic, and absorption delaying agents that are physiologically compatible. In one embodiment, the carrier is suitable for parenteral administration. Alternatively, the carrier can be suitable for intravenous, intraperitoneal, intramuscular, or sublingual administration. Pharmaceutically acceptable carriers include sterile aqueous solutions or dispersions and sterile powders for the extemporaneous preparation of sterile injectable solutions or dispersions. The use of such media and agents for pharmaceutically active substances is well known in the art. Except insofar as any conventional media or agent is incompatible with the active compound, use thereof in the pharmaceutical compositions of the disclosure is contemplated. Supplementary active compounds can also be incorporated into the compositions.

Pharmaceutical compositions typically must be sterile and stable under the conditions of manufacture and storage. The disclosure contemplates that the pharmaceutical composition is present in lyophilized form. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol), and suitable mixtures thereof. The disclosure further contemplates the inclusion of a stabilizer in the pharmaceutical composition. The proper fluidity can be maintained, for example, by the maintenance of the required particle size in the case of dispersion and by the use of surfactants.

In many cases, it will be preferable to include isotonic agents, for example, sugars, polyalcohols such as mannitol and sorbitol, or sodium chloride in the composition. Absorption of the injectable compositions can be prolonged by including an agent that delays absorption, for example, monostearate salts and gelatin. Moreover, the alkaline polypeptide can be formulated in a time-release formulation, for example in a composition that includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid, polylactic and polyglycolic copolymers (“PLG”). Many methods for the preparation of such formulations are known to those skilled in the art.

For each of the recited embodiments, the compounds can be administered by a variety of dosage forms. Any biologically acceptable dosage form known to persons of ordinary skill in the art, and combinations thereof, are contemplated. Examples of such dosage forms include, without limitation, reconstitutable powders, elixirs, liquids, solutions, suspensions, emulsions, powders, granules, particles, microparticles, dispersible granules, cachets, inhalants, aerosol inhalants, patches, particle inhalants, implants, depot implants, injectables (including subcutaneous, intramuscular, intravenous, and intradermal), infusions, and combinations thereof.

### L. Kits

In certain aspects, the instant disclosure provides kits comprising an antibody, or antigen-binding fragment thereof, of the present invention, as described herein, or an isolated nucleic acid molecule, e.g., an isolated mRNA molecule, encoding the antibody or antigen-binding fragment thereof, and a package insert with instructions to perform any of the methods described herein.

In some embodiments, the kits include instructions for using the kit. The instructions will generally include information about the use of the kit for treating and/preventing infection by SARS-CoV, SARS-COV-2, and/or another coronavirus. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters.

In some embodiments, the kit includes a pharmaceutical formulation including an antibody, or antigen-binding fragment thereof, or an isolated nucleic acid molecule, e.g., an isolated mRNA molecule, encoding the antibody or antigen-binding fragment thereof, an additional therapeutic agent, and a package insert with instructions to perform any of the methods described herein.

In some embodiments, the kit can comprise formulation components for parenteral, subcutaneous, intramuscular or intravenous administration, e.g., sealed in a vial in a form ready for loading into a syringe and administration to a subject. In some embodiments, the kits can contain one or more, e.g., two, three, four, or five or more, vials, wherein each vial contains a single unit dose for administration to a subject.

The vial can be of any size. In some embodiments, the vial is about 1 mL, about 2 mL, about 4 ml, about 8 mL, about 12 mL, about 16 mL, about 20 ml, or about 24 mL in volume.

In some embodiments, each vial comprises about 100 mg, about 200 mg, about 300 mg, about 500 mg, about 600 mg, about 700 mg, about 800 mg, about 900 mg, about 1000 mg, about 1500 mg, about 2000 mg, or about 2500 mg of the antibody or antigen-binding fragment thereof.

The kit may be packaged in a number of different configurations such as one or more containers in a single box. The different components can be combined, e.g., according to instructions provided with the kit. The components can be combined according to a method described herein, e.g., to prepare and administer a pharmaceutical composition.

In some embodiments, the kit can comprise one or more containers with appropriate positive and negative controls or control samples, to be used as standard(s) for detection, calibration, or normalization.

The kit can further comprise a second container comprising a pharmaceutically-acceptable buffer, such as (sterile) phosphate-buffered saline, Ringer's solution, or dextrose solution; and other suitable additives such as penetration enhancers, carrier compounds and other pharmaceutically acceptable carriers or excipients, as described herein. It can further include other materials desirable from a commercial and user standpoint, including other buffers, diluents, filters, and package inserts with instructions for use. The kit can also include a drug delivery system such as liposomes, micelles, nanoparticles, and microspheres. The kit can further include a delivery device, such as needles, syringes, pumps, and package inserts with instructions for use.

The above description of various illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. The teachings provided herein of the invention can be applied to other purposes, other than the examples described above.

These and other changes can be made to the invention in light of the above detailed description. In general, in the following claims, the terms used should not be construed to limit the invention to the specific embodiments disclosed in the specification and the claims. Accordingly, the invention is not limited by the disclosure, but instead the scope of the invention is to be determined entirely by the following claims.

The invention may be practiced in ways other than those particularly described in the foregoing description and examples. Numerous modifications and variations of the invention are possible in light of the above teachings and, therefore, are within the scope of the appended claims.

Certain anti-CoV-S antibody polynucleotides and polypeptides are disclosed in the sequence listing accompanying this patent application filing, and the disclosure of said sequence listing is herein incorporated by reference in its entirety.

The entire disclosure of each document cited (including patents, patent applications, journal articles, abstracts, manuals, books, or other disclosures) in the Background, Detailed Description, and Examples is herein incorporated by reference in their entireties.

The following examples are put forth so as to provide those of ordinary skill in the art with a complete disclosure and description of how to make and use the subject invention and are not intended to limit the scope of what is regarded as the invention. Efforts have been made to ensure accuracy with respect to the numbers used (e.g. amounts, temperature, concentrations, etc.), but some experimental errors and deviations should be allowed for. Unless otherwise indicated, parts are parts by weight, molecular weight is average molecular weight, temperature is in degrees centigrade; and pressure is at or near atmospheric.

### EXAMPLES

### Example 1: Recall of Pre-Existing Cross-Reactive B Cell Memory Following Omicron Breakthrough Infection

mRNA-based COVID-19 vaccines demonstrated a remarkably high degree of protective efficacy against the original SARS-COV-2 Wuhan-1 strain in clinical studies (1, 2). However, waning vaccine-induced immunity combined with the continued emergence of resistant SARS-COV-2 variants has significantly undermined vaccine effectiveness (3-5). In particular, the recently emerged Omicron variant (B.1.1.529/BA.1) and its sub-lineages (e.g., BA1.1 and BA.2) display a striking degree of antibody evasion, thus severely limiting vaccine efficacy against this VOC and allowing it to rapidly displace Delta and drive a global surge in COVID-19 caseloads (6-11).

Understanding the role of antigenic imprinting in shaping the B cell response to antigenically drifted SARS-COV-2 variants will be critical for the development of next-generation COVID-19 vaccines. Previous studies have shown that breakthrough infection with Delta or Omicron boosts serum neutralizing activity against both the Wuhan-1 vaccine strain and the infecting variant, suggesting recall of cross-reactive vaccine-induced MBCs (12-14). However, the specificities, functions, and genetic features of the antibodies mediating this response remain poorly defined. To address these questions, S-specific serological and peripheral B cell responses were investigated in a cohort of mRNA-vaccinated individuals who had recently experienced BA.1 breakthrough infections.

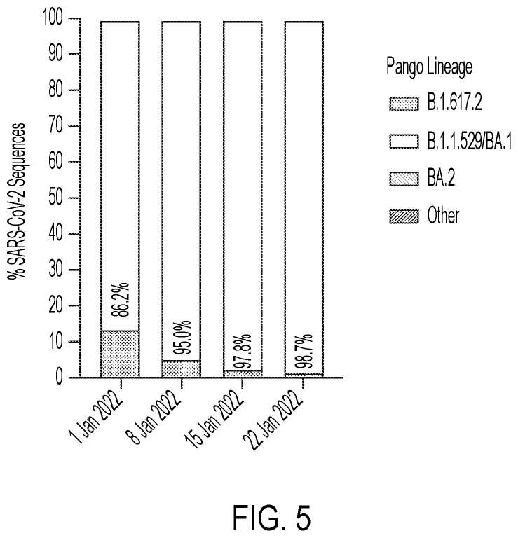

Seven mRNA (mRNA-1273 or BNT162b2)-vaccinated individuals residing in the Northeastern region of the United States who experienced SARS-COV-2 breakthrough infections between Dec. 30, 2021 and Jan. 19, 2022 were recruited (Table 1). All donors tested positive for SARS-COV-2 by RT-PCR and experienced asymptomatic or mild disease. While viral samples were unable to be obtained for genome sequencing, SARS-COV-2 variant surveillance data indicates that the BA.1 variant accounted for the vast majority of infections in the United States Northeast during this time period (FIG. 5). Breakthrough infections occurred 5-11 months after the second dose of an mRNA vaccine in four donors and one month after a third mRNA dose in three donors. To study the acute B cell response following breakthrough infection, serum and peripheral blood mononuclear cell (PBMC) samples were collected 14 to 27 days following PCR-confirmed infection (FIG. 1A).

TABLE 1
BA.1 breakthrough infection donor characteristics.

| Donor ID | IML4041 | IML4042 | IML4043 | IML4044 | IML4045 | IML4054 | IML4055 |
|---|---|---|---|---|---|---|---|
| Age | 45 | 19 | 23 | 23 | 24 | 38 | 23 |
| Sex | F | F | M | F | F | F | F |
| Vaccination | 2x | 2x | 2x | 2x | 2x | 2x mRNA- | 3x |
| History | BNT162b2 | BNT162b2 | BNT162b2 | BNT162b2 | BNT162b2, | 1273, 1x | BNT162b2 |
| | | | | | 1x | BNT162b2 | |
| | | | | | mRNA-1273 | | |
| 2nd dose | 7 May 2021 | 22 Jul. 2021 | 23 May 2021 | 10 Feb. 2021 | 15 May 2021 | 5 May 2021 | 23 May 2021 |
| vaccination date | | | | | | | |
| 3rd dose | — | — | — | — | 20 Dec. 2021 | 11 Dec. 2021 | 9 Dec. 2021 |
| vaccination date | | | | | | | |
| (if applicable) | | | | | | | |
| Date of PCR- | 31 Dec. 2021 | 4 Jan. 2022 | 30 Dec. 2021 | 2 Jan. 2022 | 6 Jan. 2022 | 19 Jan. 2022 | 6 Jan. 2022 |
| confirmed | | | | | | | |
| infection | | | | | | | |
| Days between | 25 | 21 | 26 | 23 | 19 | 14 | 27 |
| breakthrough | | | | | | | |
| infection and | | | | | | | |
| sample | | | | | | | |
| collection | | | | | | | |

Serum IgG and IgA responses to recombinant prefusion-stabilized Wuhan-1/wild type (WT) and BA.1 S proteins and RBD subunits were evaluated following breakthrough infection. For comparison, serum antibody responses were also assessed in a separate cohort of previously uninfected individuals who had received a second dose of an mRNA vaccine at either one- or six-months prior to sampling or a third mRNA booster dose one month prior to sampling (Table 2).

TABLE 2
Uninfected/mRNA-vaccinated cohort characteristics.

| | 2x mRNA (1M) | 2x mRNA (6M) | 3x mRNA (1M) |
|---|---|---|---|
| Sample size | 12 | 11 | 11 |
| Age median | 31 (21-42) | 36 (26-42) | 54 (32-58) |
| (range) | | | |
| Vaccination | 2x mRNA-1273 | 2x mRNA-1273 | 3x mRNA-1273 |
| regimens (%) | (75%) | (82%) | (64%) |
| | 2x BNT162b2 | 2x BNT162b2 | 2x mRNA-1273, |
| | (25%) | (18%) | 1x BNT162b2 |
| | | | (36%) |

Donors who experienced BA.1 breakthrough infection exhibited similar (within two-fold) serum IgG binding titers to BA.1 and WT S and RBD (FIG. 1B). In contrast, uninfected/mRNA vaccinated donors displayed two- to four-fold reduced serum IgG binding to BA.1 relative to WT S and four- to nine-fold lower serum IgG binding titers to the BA.1 RBD relative to WT (FIG. 1B). Furthermore, breakthrough infection donors exhibited significantly higher serum IgA antibody titers to both WT and BA.1 RBDs relative to uninfected/vaccinated donors (FIG. 1C). Thus, BA.1 breakthrough infection induces serum IgG and IgA binding responses to both WT and BA.1 S antigens in previously vaccinated individuals.

Next, samples were assessed for serum neutralizing activity against an ancestral SARS-COV-2 strain (D614G), as well as BA.1, Delta, and Beta VOCs using an MLV-based pseudovirus assay. Consistent with prior studies, serum samples obtained from uninfected/vaccinated donors showed 3.5- to 11-fold and 7- to 22-fold lower neutralizing titers against Beta and BA.1, respectively, relative to D614G (FIG. 1D-F). In contrast, serum samples from BA.1 breakthrough infection donors displayed similar (within two-fold) neutralizing titers against D614G and all VOCs tested, suggesting that BA.1 breakthrough infection broadens serum neutralizing antibody responses (FIG. 1G). To determine whether this breadth of activity extended to more divergent sarbecoviruses, the serum samples were also tested for neutralizing activity against SARS-COV. Similar SARS-COV neutralizing titers were observed in BA.1 breakthrough donors and uninfected/vaccinated individuals, suggesting that the breadth of serum reactivity induced by BA.1 breakthrough infection is likely limited to variants of SARS-COV-2 and not more antigenically diverse sarbecoviruses (FIG. 1D-G).

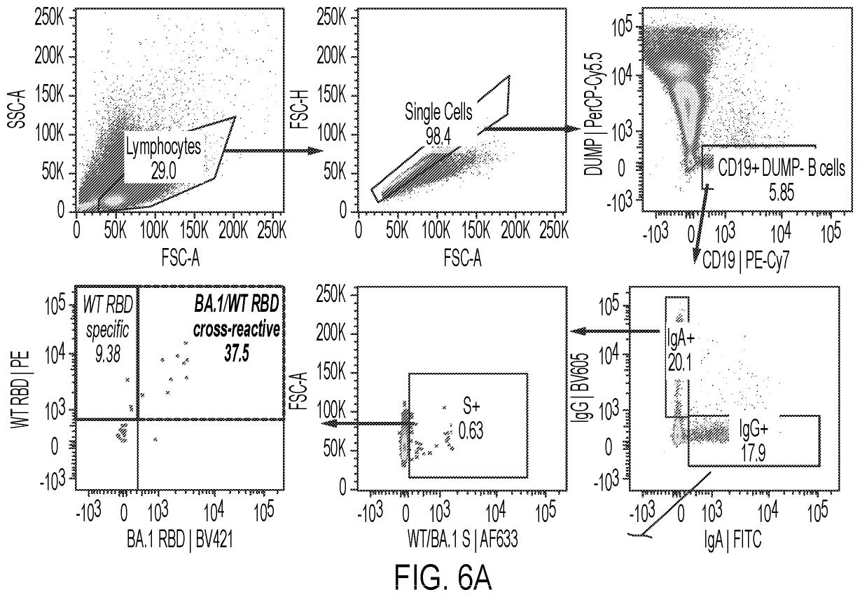

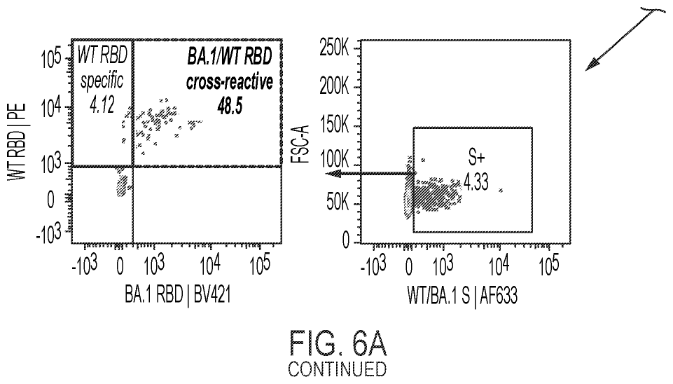

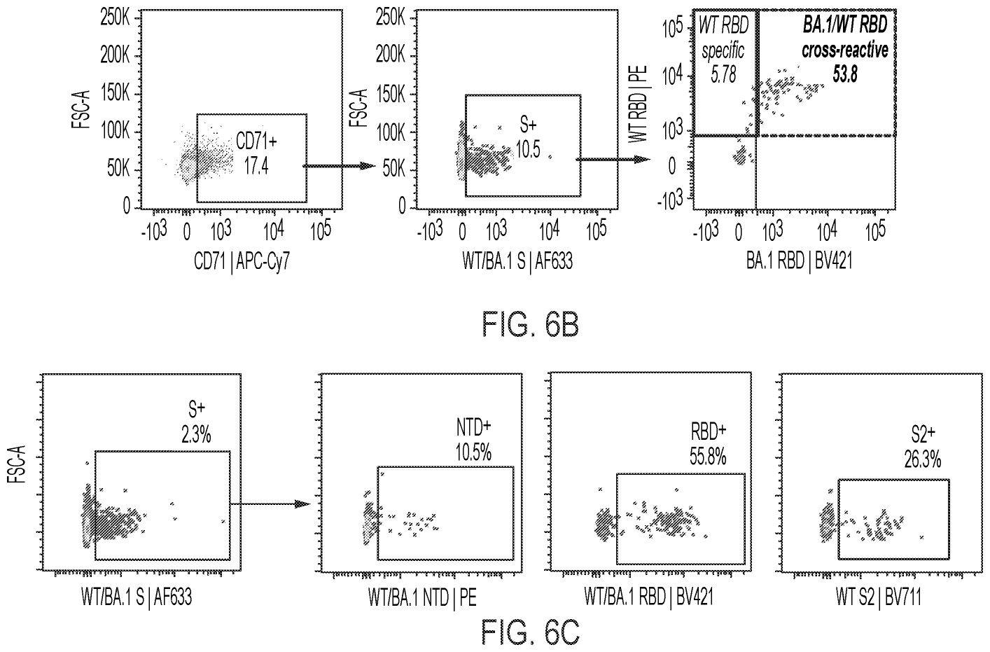

Next, the magnitude and cross-reactivity of the peripheral RBD-specific B cell response were evaluated following BA.1 breakthrough infection. Although higher serum neutralizing titers to BA.1 were observed in breakthrough donors relative to uninfected/mRNA vaccinated individuals, the two cohorts showed similar frequencies of WT- and BA.1-RBD-reactive IgG+ B cells (FIG. 2A and FIG. 6A). The limited magnitude of the circulating IgG+ B cell response following BA.1 breakthrough infection may be due to the restriction of antigen to the upper respiratory tract during mild and asymptomatic infections. To address this, the frequencies of RBD-specific IgA⁺ B cells were also compared in breakthrough donors and uninfected/vaccinated individuals. In uninfected/vaccinated donors, WT and BA.1 RBD-reactive B cells represented 0.04-0.087% and 0-0.015% of total IgA⁺ B cells, respectively (FIG. 2B). In contrast, breakthrough infection donors mounted significantly higher magnitude IgA responses to the RBD, with BA.1 RBD-specific IgA⁺ B cells accounting for 0.025-0.4% (median=0.069%) of the total IgA⁺ B cell population (FIG. 2B). It was concluded that BA.1 breakthrough infection induces similar IgG+ B cell responses and higher magnitude IgA⁺ B cell responses to BA.1 RBD antigens relative to mRNA vaccination.

To investigate the impact of pre-existing vaccine-induced immunity on the B cell response to BA.1 breakthrough infection, B cells that displayed WT/BA.1 RBD cross-reactivity were enumerated in BA.1 breakthrough donors and uninfected/mRNA vaccinated individuals (FIG. 2C, FIG. 6A). At one month post primary mRNA vaccination, only 48% of total RBD-directed B cells displayed cross-reactivity with BA.1 (FIG. 2D). The proportion of WT/BA.1 RBD cross-reactive B cells increased to 57% at 6-months post-primary vaccination and to 70% following mRNA booster immunization, consistent with the evolution of anti-SARS-COV-2 antibody breadth over time (15, 16) (FIG. 2D). Following breakthrough infection, BA.1/WT RBD cross-reactive B cells constituted 65-83% of total anti-RBD B cells and the remaining 17-35% bound only to the WT probe (FIG. 2D). Because WT RBD-specific B cells may represent resting MBCs induced by vaccination but not activated by BA. 1 infection, this analysis was also restricted to B cells expressing the activation marker CD71 (FIG. 6B). Among recently activated B cells, 87-98% of total RBD-reactive clones displayed BA.1/WT cross-reactivity, whereas the proportion of cross-reactive B cells remained unchanged in uninfected/mRNA vaccinated individuals (FIG. 2E). BA.1-specific B cells were unable to be detected in any donors following BA.1 breakthrough infection, suggesting limited induction of de novo B cell responses at this time point. Thus, it was concluded that BA.1 breakthrough infection preferentially activates B cells that display cross-reactivity with both BA. 1 and the original Wuhan-1 vaccine strain.

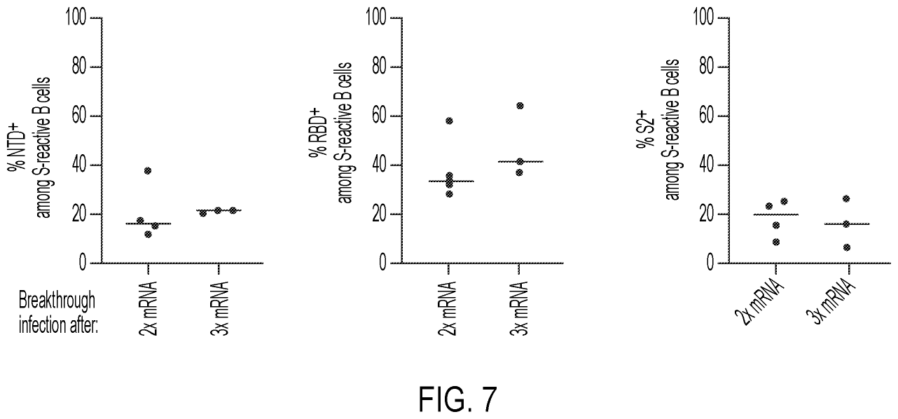

Next, it was evaluated whether BA.1 breakthrough infection modifies the immunodominance hierarchy of B cells targeting each subdomain with the S trimer. To calculate the proportion of full-length S-reactive B cells targeting each subdomain, B cells were stained with differentially labeled tetramers of full-length S, RBD, NTD, and prefusion-stabilized S2 (FIG. 6C). In the uninfected/vaccinated cohort, class-switched B cells targeting the NTD, RBD, and S2 subdomains comprised 18%, 25%, and 37% of the total S-directed response, respectively, and these proportions remained largely unchanged at six months post-vaccination and after mRNA booster immunization (FIG. 2F-H). In contrast, significantly higher proportions of RBD-directed B cells were observed among donors with breakthrough infection, ranging from 35-63% (median=46%) of the total activated (CD71⁺) B cell response to S (FIG. 2G). Furthermore, S2-reactive B cells comprised a smaller fraction (median=16%) of the S-specific response in breakthrough donors relative to uninfected/mRNA vaccinated individuals (FIG. 2H). This altered immunodominance pattern was observed in donors experiencing BA.1 breakthrough infection following both second and third dose mRNA vaccination (FIG. 7). In summary, BA.1 breakthrough infection appears to re-direct B cell immunodominance hierarchy from the S2 subunit to the RBD.

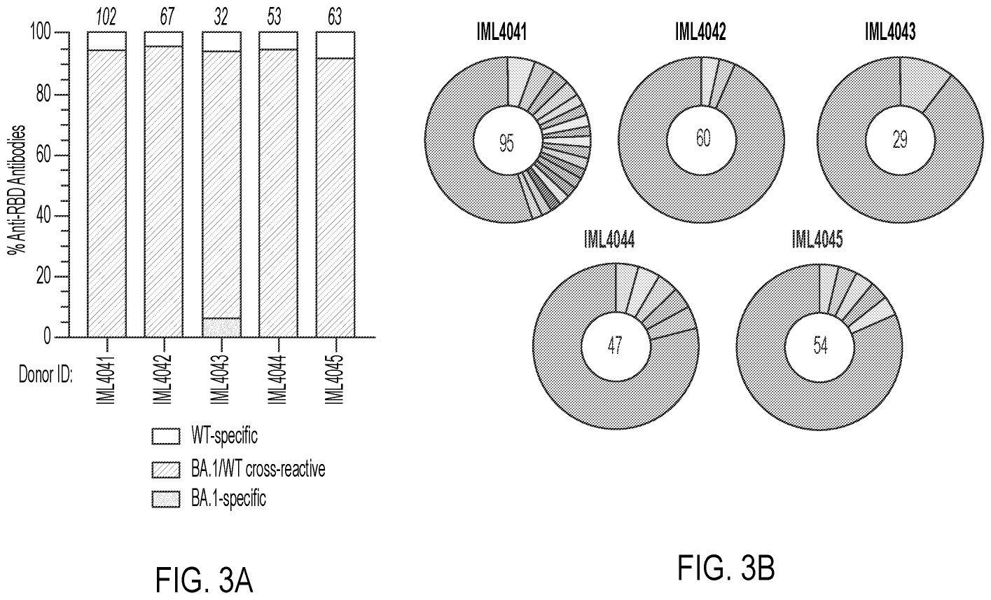

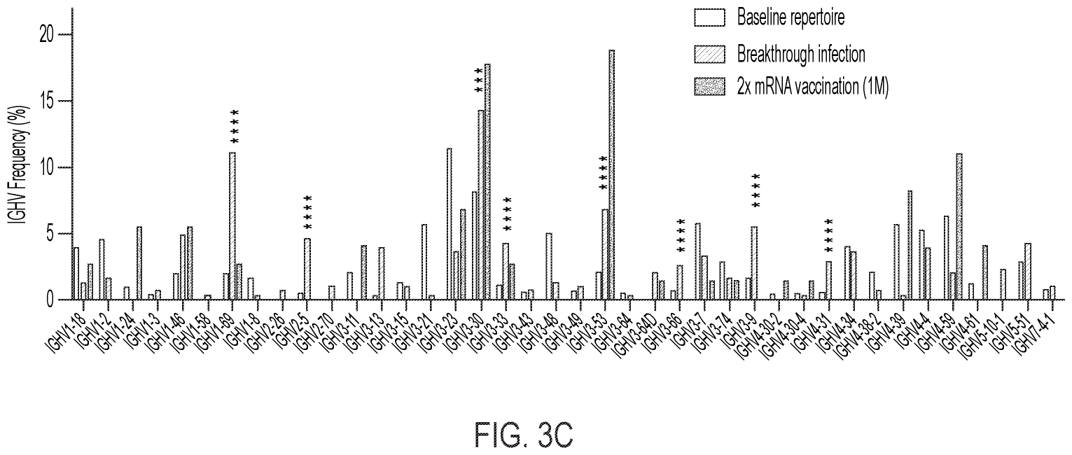

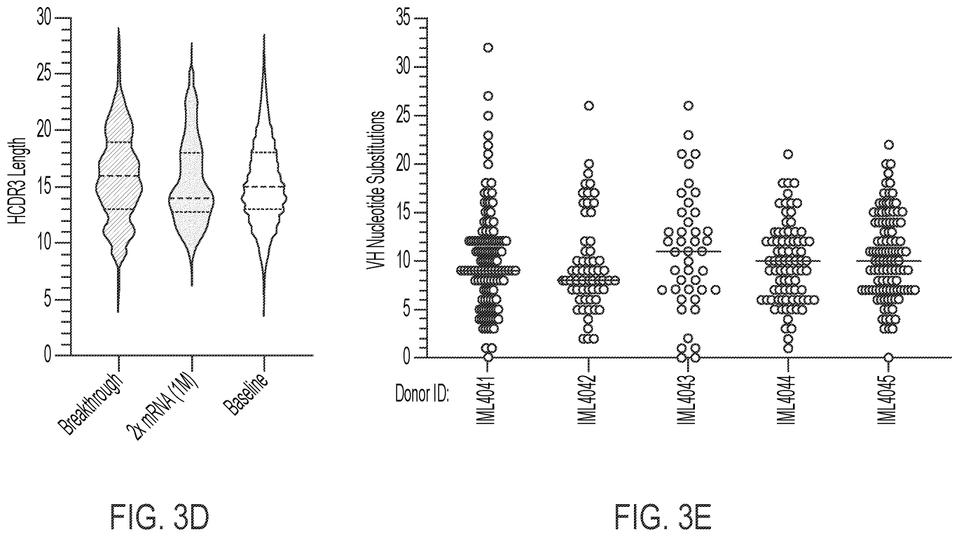

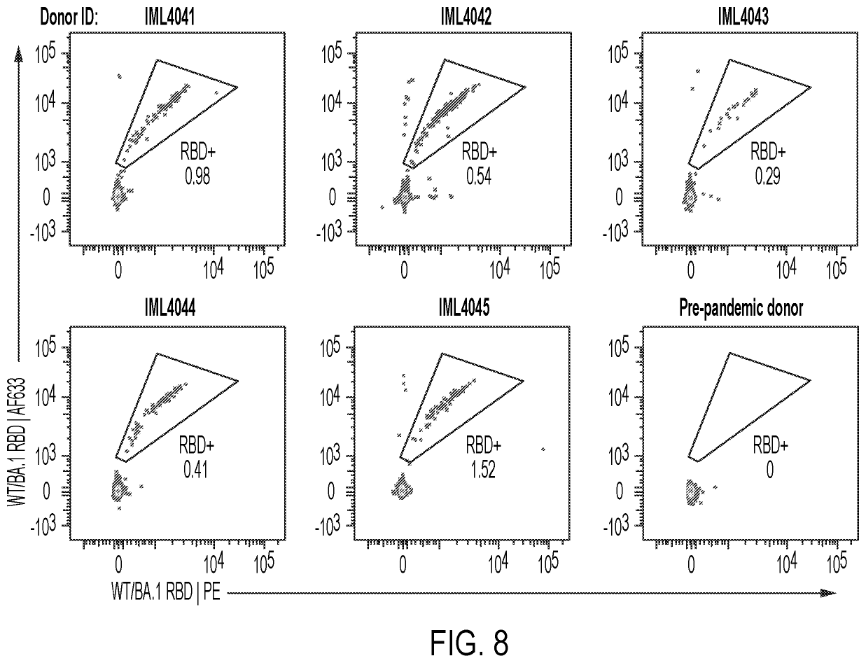

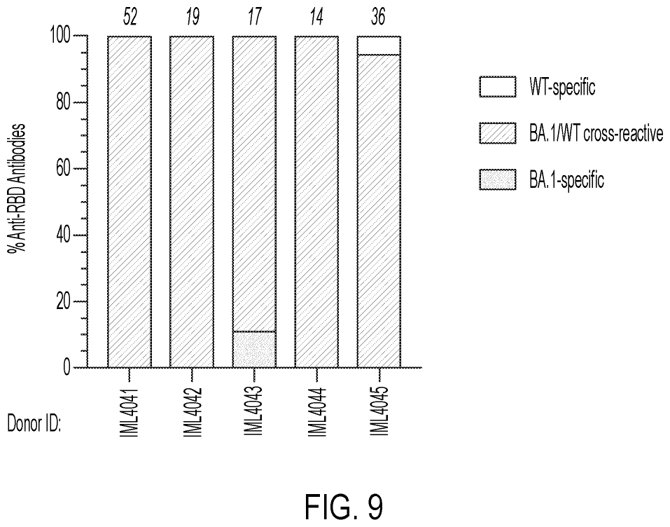

To characterize the molecular features of anti-RBD antibodies elicited by BA.1 breakthrough infection, 410 class-switched RBD⁺ B cells were single-cell sorted from five breakthrough infection donors and expressed 317 natively paired antibodies as full-length IgGs (32 to 102 antibodies per donor) (FIG. 8). Despite sorting with a mixture of WT and BA.1 RBDs, the vast majority of IgGs displayed BA.1 RBD reactivity (92-96%), providing strong evidence that these donors experienced BA.1 infections (FIG. 3A). In addition, index sorting analysis revealed that all antibodies derived from CD71⁺ B cells recognized BA.1, suggesting that the WT-specific antibodies likely originated from resting MBCs elicited by vaccination (FIG. 9).

Sequence analysis revealed that the BA.1 RBD-reactive antibodies displayed a relatively high level of clonal diversity, with 7-45% belonging to expanded clonal lineages (FIG. 3B). A significant over-representation of heavy chain germline genes IGHV3-53, 3-66, 3-30, 1-69, 3-9, and 4-31 was observed in BA.1 breakthrough infection repertoires relative to the baseline human repertoire (17) (FIG. 3C). While IGHV3-53, 3-66, and 3-30 germline gene families have also been shown to be over-represented in the antibody response to ancestral SARS-COV-2 strains, IGHV1-69, 3-9, and 4-31 appear to be unique to the BA.1 breakthrough response (18) (FIG. 3C). The BA.1 RBD-reactive antibodies displayed a similar HCDR3 length distribution compared to the baseline repertoire (FIG. 3D). Ninety-five to 100% of antibodies derived from each donor contained somatic mutations, with median SHM levels ranging from 8 to 11 nucleotide substitutions in VH, supporting a memory B cell origin (FIG. 3E). It was concluded that the early B cell response to breakthrough infection is dominated by highly mutated clones that cross-react with both WT and BA.1 RBDs.

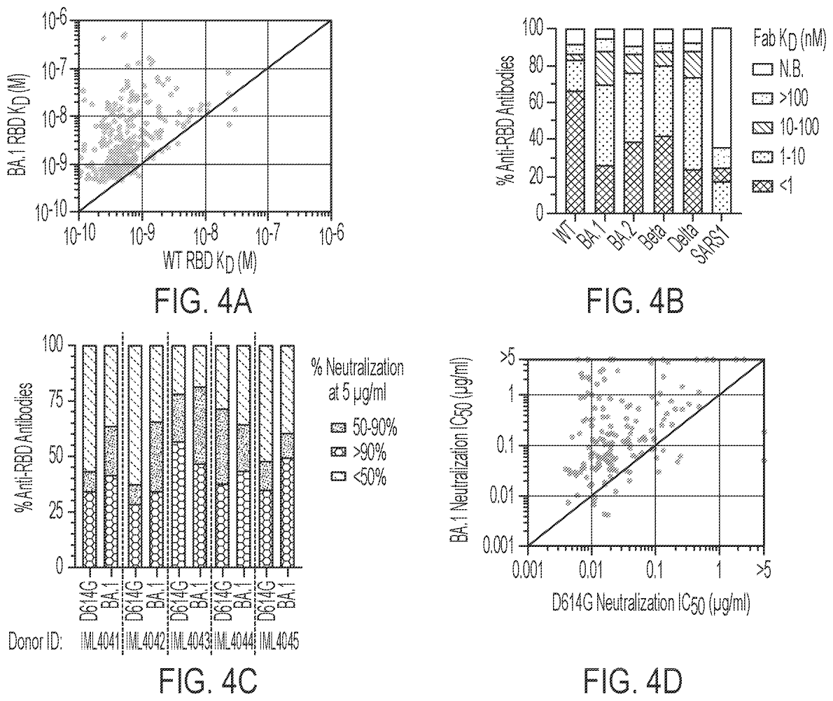

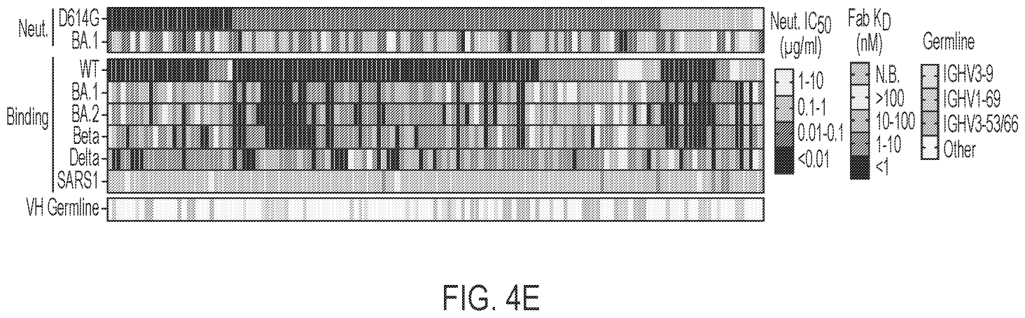

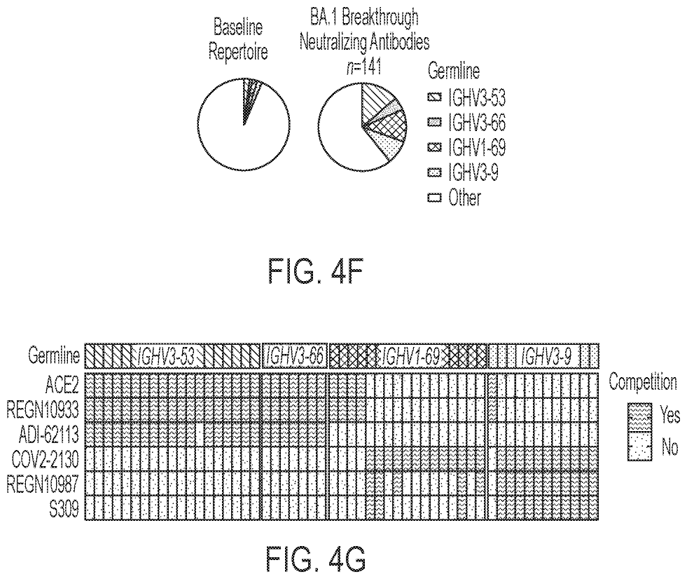

To further evaluate the binding properties of the BA.1 RBD-reactive antibodies, their monovalent binding affinities for SARS-COV-2 D614G, BA.1, BA.2, Beta, and Delta RBDs and the SARS-COV RBD were measured. The majority (204/293) of RBD-directed antibodies bound with high affinity (K_{D}<10 nM) to both BA.1 and WT RBDs, supporting selection from an affinity matured B cell population (FIG. 4A). However, approximately 70% of the antibodies displayed higher affinity binding (>2-fold) to the WT RBD relative to BA.1, providing strong evidence for the re-activation of pre-existing vaccine-induced MBCs following BA.1 breakthrough infection (FIG. 4A). In contrast to vaccine-induced anti-RBD antibodies, which often show reduced activity against the Beta VOC, only a minority (<5%) of antibodies derived from BA.1 breakthrough donors displayed loss of binding to Beta relative to WT (10, 16) (FIG. 4B). This difference in antibody binding cross-reactivity is likely due to the presence of shared mutations within the Beta and BA.1 RBDs (E484K/A, K417N, and N501Y). Overall, 82% (241/293) of anti-RBD antibodies isolated from breakthrough infection donors displayed monovalent binding to WT, Beta, Delta, BA.1, and BA.2 RBDs, suggesting that BA.1 breakthrough infection activates a large proportion of B cells with broad SARS-COV-2 VOC recognition (FIG. 4B). Consistent with the weak serum neutralizing activity observed against SARS-COV, less than 10% of RBD-targeting antibodies exhibited detectable monovalent binding to the SARS-COV RBD (FIG. 4B). Thus, BA.1 breakthrough infection appears to preferentially expand B cells targeting epitopes that are conserved across SARS-COV-2 variants but not more antigenically divergent sarbecoviruses.

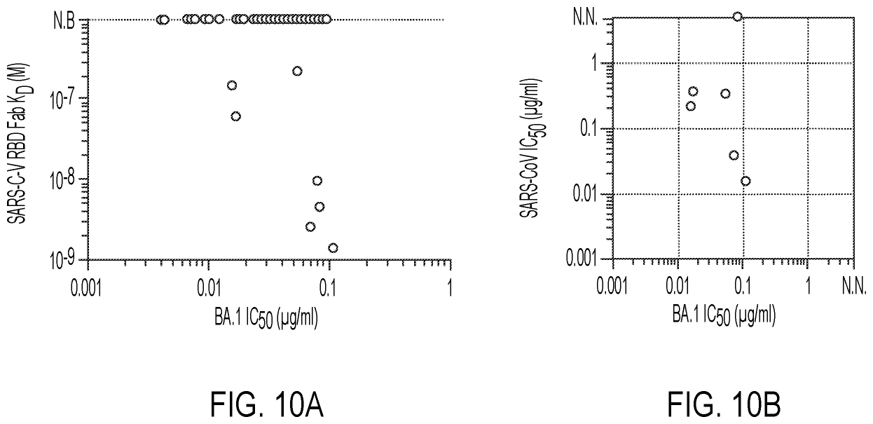

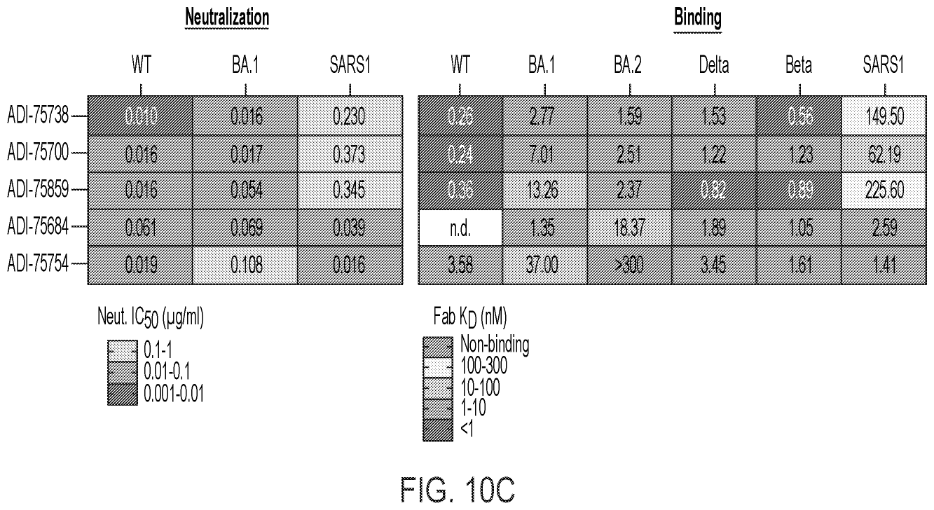

Next, the BA.1 RBD-reactive antibodies were screened for neutralizing activity against D614G and BA.1. Twenty-eight to 56% and 34-49% of antibodies from each donor displayed >90% neutralizing activity against D614G and BA.1, respectively, at a concentration of 5 μg/ml (FIG. 4C). Titration of the neutralizing antibodies against D614G and BA.1 revealed that 45% (64/141) potently neutralize both viruses with IC₅₀s less than 0.1 μg/ml (FIG. 4D). Consistent with their overall increased binding affinities for the WT RBD relative to BA.1, most of the neutralizing antibodies (78%) displayed higher potency against D614G compared to BA.1 (FIG. 4D). Notably, a large proportion of BA.1 neutralizing antibodies also displayed cross-reactivity with Delta (79%), Beta (90%), and BA.2 (86%) RBDs with affinities within 10-fold of BA.1 (FIG. 4E). A limited number of these VOC cross-reactive antibodies (5 out of 141) also neutralized SARS-COV, with IC₅₀s ranging from 0.039-0.35 μg/ml (FIG. 10). It was concluded that BA.1 breakthrough infection elicits RBD-directed antibodies with broad activity against SARS-COV-2 VOCs.

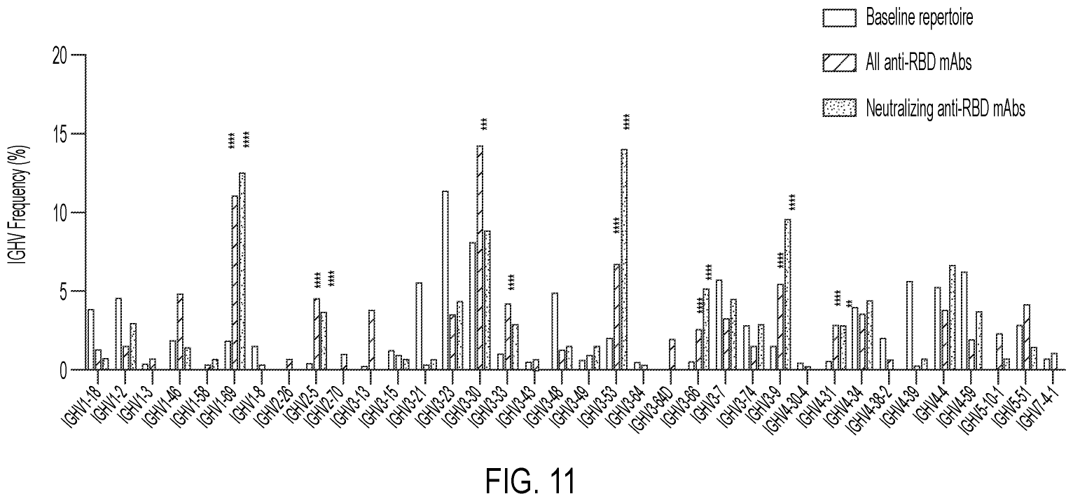

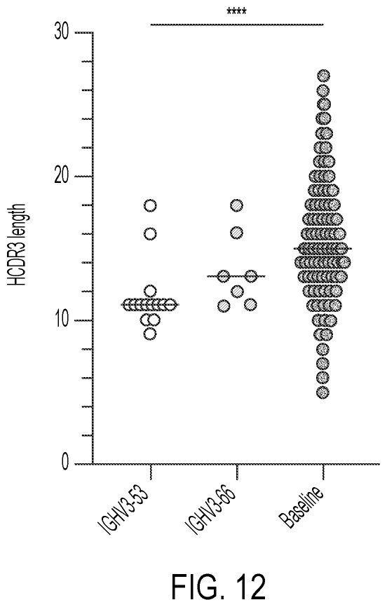

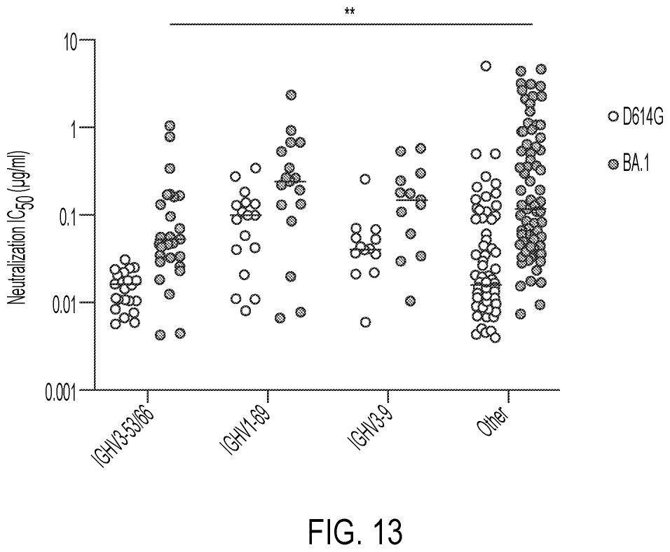

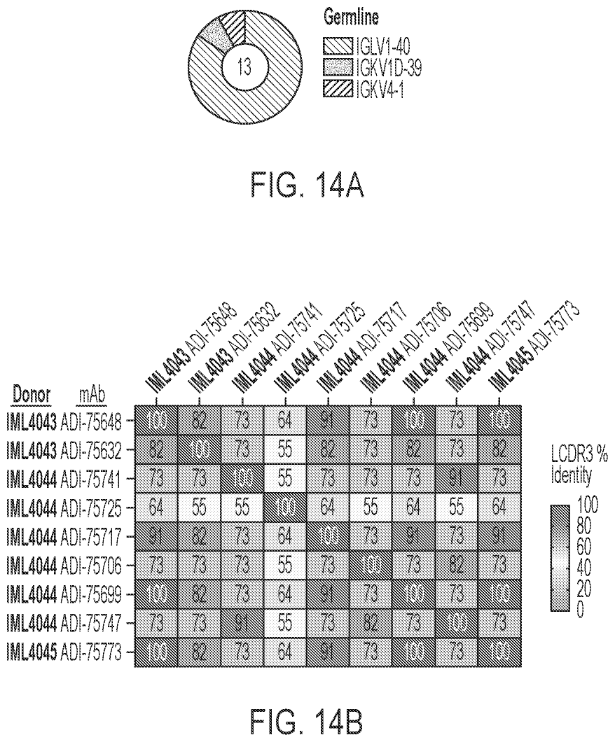

Previous studies have defined several “public” classes of neutralizing antibodies (Class 1-4) induced by SARS-COV-2 infection and vaccination (19, 20). To determine whether BA.1 breakthrough infection also elicits recurrent neutralizing antibody responses, the sequence and binding features of the BA.1 neutralizing antibodies were analyzed. Over 40% of all BA. 1 neutralizing antibodies utilized one of three VH germline genes (IGHV3-53/66, IGHV1-69, and IGHV3-9) (FIG. 4F and FIG. 11). Similar to previously described IGHV3-53/66 antibodies isolated from mRNA-vaccinated individuals, the BA.1 neutralizing IGHV3-53/66 antibodies possessed short HCDR3s (11-12 residues) and displayed competitive binding with ACE2, the class 1 mAb REGN10933, and the COVA1-16-like class 4 mAb ADI-62113 (21) (FIG. 4G and FIG. 12). However, unlike vaccine-induced IGHV3-53/66 antibodies, which generally lack activity against SARS-COV-2 variants containing substitutions at position K417 (e.g. Beta, Gamma, and BA.1), breakthrough infection-derived IGHV3-53/66 antibodies displayed broad reactivity with all VOCs tested and potently neutralized both D614G and BA.1 pseudoviruses (median IC₅₀=0.016 and 0.051 μg/ml, respectively) (22, 23) (FIG. 4E and FIG. 13). Thus, these IGHV3-53/66-utilizing antibodies appear to recognize an antigenic site that is overlapping but distinct from previously described IGHV3-53/66 antibodies induced by infection and vaccination.

Neutralizing antibodies utilizing the IGHV1-69 and IGHV3-9 germline genes also broadly recognized SARS-COV-2 variants, including BA.2 (FIG. 4E). In contrast to IGHV3-53/66, these germline genes have not been shown to be over-represented among RBD-directed antibodies identified from mRNA-vaccinated donors (FIG. 3C). Antibodies utilizing the IGHV1-69 germline gene segregated into two groups, one comprised of antibodies that targeted an ACE2- and REGN10933-competitive region and the other containing antibodies that recognized a non-ACE2 competitive site overlapping the COV2-2130 (class 3) epitope (FIG. 4G). Notably, >80% of non-ACE2 competitive clones utilized the light chain IGLV1-40 gene and displayed highly similar LCDR3 sequences, suggesting a convergent mode of recognition (FIG. 14). Finally, 12/13 IGHV3-9 antibodies recognized an epitope outside of the ACE2 binding site and competed with all three class 3 antibodies tested (S309, REGN10987, and COV2-2130), suggesting a binding mode distinct from the IGHV1-69 antibodies (FIG. 4G). Taken together, BA.1 breakthrough infection elicits multiple recurrent classes of anti-RBD antibodies with broad SARS-COV-2 VOC reactivity.

A deep understanding of how pre-existing SARS-COV-2 immunity shapes the B cell response to heterologous variant exposure will be important for the development of variant-based booster vaccines. Here this example demonstrates that the acute B cell response to BA.1 breakthrough infection is primarily mediated by re-activated vaccine-induced memory B cell clones with broader SARS-COV-2 VOC cross-reactivity than those elicited by infection or vaccination with ancestral SARS-COV-2 strains. Although the durability and kinetics of the breakthrough-activated B cell response remain unknown, the induction of cross-reactive responses following BA.1 breakthrough infection suggests that booster immunization with heterologous S proteins may be a promising strategy for the elicitation of broadly neutralizing responses against future emerging VOCs.

Despite the immunodominance of the S2 subunit in the context of primary SARS-COV-2 infection and vaccination, BA.1 breakthrough infection preferentially boosted cross-reactive antibodies targeting the antigenically variable and immuno-subdominant RBD. The molecular explanation(s) for this shift in B cell immunodominance hierarchy remain to be determined but may be driven by increased serum antibody masking of the conserved S2 subunit relative to the more divergent RBD, resulting in limited S2 epitope accessibility for B cell targeting. Notably, serum antibody feedback via epitope masking has been previously shown to limit B cell responses to immunodominant viral epitopes and permit expansion of subdominant responses (24).

Finally, several monoclonal antibodies from BA.1 breakthrough infection donors were identified that display broad activity against all SARS-COV-2 VOCs described to date as well as SARS-CoV. These antibodies represent promising candidates for therapeutic development and provide a framework for the development of vaccines that induce broadly neutralizing antibody responses.

### Methods and Materials

### Human Subjects and Blood Sample Collection.

Breakthrough infection donors and uninfected two-dose vaccinated donors participated with informed consent under the healthy donor protocol D10083, Immune Monitoring Core (DartLab) Laboratory at Dartmouth-Hitchcock Hospital. Uninfected three-dose vaccinated participants are enrolled in the clinical trial, CoVacc—Immune response to vaccination against Covid-19, an open multicenter phase IV study, was approved by the Swedish Ethics Review Authority (Dnr 2021-00055) and the Medical Products Agency Sweden. The study was registered at European Clinical Trials Database (EUDRACT Number 2021-000683-30) before the first patient was enrolled. Umeå University, Sweden served as trial sponsor and the Clinical Research Center, University Hospital of Northern Sweden was monitoring the study for regulatory compliance. Individuals were included after informed consent and data were stored in accordance with the EU General Data Protection Regulation.

Seven participants with BA.1 breakthrough infection were recruited to participate in this study. SARS-COV-2 infection was determined by positive results via both RT-PCR from a saliva sample and rapid antigen test from a nasal swab sample. All participants were previously immunized with two- or three-doses of an mRNA vaccine (BNT162b2 or mRNA-1273) and had no documented history of SARS-COV-2 infection prior to vaccination. Clinical and demographic characteristics of breakthrough infection donors are shown in Table 1. Participants presented to the Dartmouth-Hitchcock Hospital (D-HH) 14 to 27 days after their first SARS-COV-2 positive test for blood draw. Venous blood was collected using BD Vacutainer® tubes with acid citrate dextrose (ACD), and plasma and PBMCs were isolated using a Ficoll 1077 (Sigma) gradient, washed, and counted with an anti-human CD45 stain on a volumetric flow cytometer. PBMC were frozen in 12.5% human serum and 10% DMSO diluted in RPMI-1040 and stored in liquid nitrogen until use. Plasma was isolated and frozen at −80° C.

A separate cohort of uninfected/mRNA-vaccinated volunteers were recruited for blood sample collection at D-HH (for two-dose mRNA-vaccinated donors) and Umeå University (for three-dose mRNA-vaccinated donors). Samples were collected at one month (n=12) and six months (n=11) following the second mRNA dose or one month following the third mRNA dose (n=11). Demographics for these participants are shown in Table 2. Sample collection and processing methods for two-dose vaccinated individuals are described above. For individuals who received a third mRNA dose, venous blood was collected and PBMCs and plasma isolated in BD EDTA Vacutainer® CPT™ tubes. PBMCs were frozen in 90% fetal calf serum supplemented with 10% DMSO and stored in liquid nitrogen until use. Plasma and serum were stored at −80° C.

### Recombinant SARS-COV-2 S Production.

To produce prefusion-stabilized WT SARS-COV-2 HexaPro S, DNA encoding residues 1-1208 of the SARS-COV-2 spike (Genbank NC NC_045512.2) with substitutions F817P, A892P, A899P, A942P, K986P, V987P, “GSAS” mutations (SEQ ID NO:7) from positions 682-685 and a C-terminal T4 fibritin motif, 8×HisTag (SEQ ID NO:8) and TwinStrepTag (SARS-COV-2 S-2P) was cloned into a pcDNA3.4 vector. The following mutations were additionally cloned into the Omicron/BA.1 HexaPro S plasmid: A67V, Δ69-70, T95I, G142D, Δ143-145, Δ211, L212I, ins214EPE, G339D, S371L, S373P, S375F, K417N, N440K, G446S, S477N, T478K, E484A, Q493K, G496S, Q498R, N501Y, Y505H, T547K, D614G, H655Y, N679K, P681H, N764K, D796Y, N856K, Q954H, N969K, L981F. Plasmids were transiently transfected into FreeStyle HEK 293F cells (Thermo Fisher) using polyethylenimine following the manufacturer's directions. After one week of culture, the supernatants were harvested, and centrifuged to remove cellular debris. S protein preps were purified by Ni affinity chromatography and followed by size exclusion chromatography using the Superose 6 column (GE Healthcare) before concentrating and freezing at −80° C.

### Serum ELISAs

96-well half-area plates (Corning) were coated with the following recombinant antigens at a concentration of 5 μg/ml diluted in PBS: SARS-COV-2 WT Hexapro-stabilized S, BA.1 Hexapro-stabilized S, WT RBD (Sino Biological, Cat #40592-V08B), BA.1 RBD (Acro Biosystems, Cat #SPD-C522e), WT NTD (Acro Biosystems, Cat #S1D-52H6), BA.1 NTD (Acro Biosystems, Cat #SPD-C522d), and Hexapro-stabilized S2 (Acro Biosystems, Cat #S2N-C52H5) antigens. Following overnight incubation at 4° C., wells were washed with wash buffer (1×PBS with 0.05% Tween-20) and blocked with 75 μl 3% bovine serum albumin (BSA) in 1×PBS for 1 h at 37° C. Coated wells were subsequently incubated with serial dilutions of human sera ranging from 1:40 to 1:1,310,720 in a solution of 0.1% BSA, 0.01% Tween-20 in 1×PBS for 1 h at 37° C. and then washed three times with wash buffer. To detect antigen-specific IgG and IgA, wells were incubated with either a 1:5000 dilution of anti-human IgG horseradish peroxidase (HRP; Jackson Immunoresearch Laboratories, Cat #109-036-098) or a 1:10,000 dilution of anti-human IgA HRP (Jackson Immunoresearch Laboratories, Cat #109-036-011) in 0.1% BSA, 0.01% Tween-20, 1×PBS for 1 h at 37° C. Plates were then washed three times and developed with 25 μl of room temperature-equilibrated 1-Step™ Ultra TMB Substrate Solution (Thermo Fisher Scientific) for 5 min. The developing reaction was terminated by addition of 25 μl 4 N sulfuric acid. Absorbance was measured at 450 nm using a Spectramax microplate reader (Molecular Devices). Titration curves were fitted via non-linear regression to determine the 50% effective concentration (EC₅₀) in GraphPad Prism (version 9.3.1).

### SARS-COV-2 Pseudovirus Generation.

Single-cycle infection pseudoviruses were generated as previously described (25). Briefly, HEK293T cells seeded overnight in 6-well tissue culture plates (Corning) were co-transfected with the following plasmids: 1) 0.5 μg of pCDNA3.3 encoding SARS-COV-2 spike genes with 19-residue C-terminal truncations, 2) 2 μg of MLV-based luciferase reporter gene plasmid (Vector Builder), and 3) 2 μg of MLV gag/pol (Vector Builder). The SARS-COV-2 Omicron/BA.1 contained the following mutations in relative to Wuhan-1: A67V, A69-70, T95I, G142D, A143-145, A211, L212I, ins214EPE, G339D, S371L, S373P, S375F, K417N, N440K, G446S, S477N, T478K, E484A, Q493K, G496S, Q498R, N501Y, Y505H, T547K, D614G, H655Y, N679K, P681H, N764K, D796Y, N856K, Q954H, N969K, L981F. Plasmids were combined with Lipofectamine 2000 (ThermoFisher Scientific) and transfected following the manufacturer's recommendations. Culture supernatants containing SARS-CoV-2 S-pseudotyped MLV particles were collected 48 h post-transfection, aliquoted, and frozen at −80° C. for neutralization assays.

### Pseudovirus Neutralization Assay.

HeLa-hACE2 reporter cells (BPS Bioscience Cat #79958) were seeded overnight at 10,000 cells per well in 96-well tissue culture plates (Corning). Human plasma and serum samples were heat-inactivated at 56° C. for 30 min. Next, monoclonal antibodies or heat-inactivated sera were serially diluted in MEM/EBSS media supplemented with 10% FBS with 50 μl of MLV viral stock and incubated for 1 h at 37° C. with 5% carbon dioxide. Cell culture media was removed, and cells were washed two times with PBS. The virus-antibody mixture was subsequently added to HeLa-hACE2 cells and incubated for 48 h at 37° C. with 5% carbon dioxide. Cells were then lysed with Luciferase Cell Culture Lysis 5× reagent (Promega), and luciferase activity was measured using the Luciferase Assay System (Promega) following manufacturer's protocols. Infectivity was measured as relative luminescence units (RLUs) using a luminometer (Perkin Elmer). The percentage neutralization was calculated as 100*(1−[RLUsample−RLUbackground]/[RLUisotype control mAb−RLUbackground]), and the 50% neutralization concentration was interpolated from four-parameter non-linear regression fitted curves in GraphPad Prism (version 9.3.1).

### FACS Analysis of SARS-COV-2 S-Specific B Cell Responses.

Antigen-specific B cells were detected using recombinant biotinylated antigens tetramerized with fluorophore-conjugated streptavidin (SA). For detection of peripheral B cells that recognize WT and/or BA.1 RBD, 4:1 molar ratios of biotinylated antigens to SA were mixed in the following combinations: WT HexaPro S with SA-AlexaFluor 633 (Invitrogen), BA.1 HexaPro S with SA-AlexaFluor 633 (Invitrogen), WT RBD (Acro Biosystems, Cat #SPD-C82E8) with SA-BV421 (BioLegend), and BA.1 RBD (Acro Biosystems, Cat #SPD-C522e) with SA-phycoerythrin (PE; Invitrogen). For determination of subdomain reactivities within the total S-specific B cell population, antigen tetramers were mixed in the following combinations: WT HexaPro S with SA-AlexaFluor 633 (Invitrogen), BA.1 HexaPro S with SA-AlexaFluor 633 (Invitrogen), WT RBD (Acro Biosystems, Cat #SPD-C82E8) with SA-BV421 (BioLegend), BA.1 RBD (Acro Biosystems, Cat #SPD-C522e) with SA-BV421, WT NTD (Acro Biosystems, Cat #S1D-52H6) with SA-PE, BA.1 NTD (Acro Biosystems, Cat #SPD-C522d) with SA-PE, and HexaPro-stabilized WT S2 (Acro Biosystems, Cat #S2N-C52H5) with SA-BV711 (BD BioSciences). Antigen tetramers were incubated for 30 min at 4° C., followed by quenching of unbound SA sites using 5 μl of 2 μM Pierce biotin (ThermoFisher Scientific). PBMCs were stained with pooled tetramerized antigens (25 nM each) and anti-human antibodies anti-CD19 (PE-Cy7; Biolegend), anti-CD3 (PerCP-Cy5.5; Biolegend), anti-CD8 (PerCP-Cy5.5; Biolegend), anti-CD14 (PerCP-Cy5.5; Invitrogen), and anti-CD16 (PerCP-Cy5.5; Biolegend) diluted in a 1:1 [v/v] mixture of Brilliant Stain Buffer (BD BioSciences) and FACS buffer (2% BSA/1 mM EDTA in 1×PBS) for 15 min on ice. Following one wash, cells were resuspended in a mixture of propidium iodide and anti-human antibodies anti-IgG (BV605; BD Biosciences), anti-IgA (FITC; Abcam), anti-CD27 (BV510; BD Biosciences), and anti-CD71 (APC-Cy7; Biolegend) and incubated for 15 min on ice. After washing two times with FACS buffer, samples were analyzed using a BD FACS Aria II (BD Biosciences).

The proportion of class-switched RBD-specific B cells that reacted with WT and/or BA.1 RBD was calculated by dividing the number of BA.1/WT cross-reactive or WT-specific IgG+ and IgA+ (swIg+) B cells by the total number of RBD+ S+ swIg+ B cells. The proportion of S-reactive B cells that recognized each subdomain (NTD, RBD, or S2) was calculated by dividing the number of IgG+ and IgA+ (swIg+) B cells that recognize both S and the subdomain by the total number of S+ swIg+ cells.

### Single B Cell Sorting.

Biotinylated recombinant WT (Acro Biosystems, Cat #SPD-C82E8) and BA.1 (Acro Biosystems, Cat #SPD-C522e) RBDs were separately mixed with AlexaFluor 633-conjugated SA (SA-633; Invitrogen) and PE-SA (Invitrogen) at a 4:1 molar ratio of antigen to SA for 30 min at 4° C. The four antigen-SA pairs were then pooled to create a mixture of PE- and APC-labelled WT and BA.1 RBD tetramers. PBMCs were stained with tetramerized antigens (25 nM each) and a mixture of anti-CD19 (PE-Cy7; Biolegend), anti-CD20 (BV711; Biolegend) anti-CD3 (PerCP-Cy5.5; Biolegend), anti-CD8 (PerCP-Cy5.5; Biolegend), anti-CD14 (PerCP-Cy5.5; Invitrogen), and anti-CD16 (PerCP-Cy5.5; Biolegend) antibodies diluted in FACS buffer (2% BSA/1 mM EDTA in 1×PBS) for 15 min on ice. Stained cells were centrifuged for 10 min at 400×g, washed once with FACS buffer, and centrifuged again to pellet the cells. Next, cells were resuspended in propidium iodide and anti-human antibodies anti-IgM (BV421; BD Biosciences), anti-IgG (BV605; BD Biosciences), anti-IgA (FITC; Abcam), anti-CD27 (BV510; BD Biosciences), and anti-CD71 (APC-Cy7; Biolegend) diluted in Brilliant Stain Buffer (BD BioSciences) and FACS buffer. Following 15 min of incubation on ice, Cells were washed two times, resuspended in FACS buffer, and analyzed using a BD FACS Aria II (BD Biosciences). Class-switched B cells, defined as CD19+CD3−CD8−CD14−CD16−PI−IgM− and IgG+ or IgA+, that specifically bound to the WT/BA. 1 RBD mixture were single-cell index sorted into 96-well polystyrene microplates (Corning) containing 20 μl lysis buffer per well [5 μl of 5× first strand SSIV cDNA buffer (Invitrogen), 1.25 μl dithiothreitol (Invitrogen), 0.625 μl of NP-40 (Thermo Scientific), 0.25 μl RNaseOUT (Invitrogen), and 12.8 μl dH2O]. Plates were then frozen at −80° C. before further downstream processing.

### Amplification and Cloning of Antibody Variable Genes.

Antibody variable gene mRNA transcripts (VH, Vk, Vλ) were amplified by RT-PCR as described previously (22). Briefly, cDNA was synthesized using SuperScript IV enzyme (ThermoFisher Scientific), followed by two rounds of nested PCRs. The second cycle of nested PCR added 40 base pairs of 5′ and 3′ homology to restriction enzyme-digested S. cerevisiae expression vectors to enable homologous recombination during transformation. PCR-amplified variable gene DNA was chemically transformed into competent yeast cells via the lithium acetate method and yeast were plated on selective amino acid drop-out agar plates (26). Transformed yeast colonies were picked for sequencing and characterization.

### Expression and Purification of IgG and Fab Molecules.

Antibodies were expressed as human IgG1 via S. cerevisiae cultures, as described previously (22). Briefly, yeast cells were grown for IgG expression over 6 days, and the IgG-containing supernatant was subsequently harvested by centrifugation. Antibodies were purified by protein A-affinity chromatography, eluted with a solution of 200 mM acetic acid/50 mM NaCl (pH 3.5). The pH was then neutralized using ⅛th volume of 2 M Hepes (pH 8.0).

Fab fragments were generated by incubating IgG with papain for 2 h at 30° C. The reaction was terminated using iodoacetamide, and the mixture containing digested Fab and Fc was purified by Protein A agarose to remove Fc fragments and undigested IgG. Fabs present in the flow-through were further purified using CaptureSelect™ IgG-CH1 affinity resin (ThermoFisher Scientific) and eluted from the column using 200 mM acetic acid/50 mM NaCl (pH 3.5). Fab solutions were pH-neutralized using ⅛th volume 2 M Hepes (pH 8.0).

### Binding Affinity Measurements by Biolayer Interferometry.

Binding affinities were measured by biolayer interferometry (BLI) using a FortéBio Octet HTX instrument (Sartorius). All steps were performed at 25° C. and at an orbital shaking speed of 1000 rpm. All reagents were formulated in PBSF buffer (PBS with 0.1% w/v BSA).

Recombinant biotinylated antigens were diluted (100 nM) in PBSF and loaded onto streptavidin biosensors (Sartorius) to a sensor response of 0.6-1.0 nm and then allowed to equilibrate in PBSF for a minimum of 30 min. After a 60 s baseline step in PBSF, antigen-loaded sensors were exposed (180 s) to Fab or IgG fragments (100 nM) and then dipped (180 s) into PBSF to measure any dissociation of the antigen from the biosensor surface. Fab binding data with detectable binding responses (>0.1 nm) were aligned, inter-step corrected (to the association step) and fit to a 1:1 binding model using the FortéBio Data Analysis Software, version 11.1.

### Epitope Binning by Biolayer Interferometry.

Antibody competition with recombinant human ACE2 and comparator antibodies for binding to SARS-COV-2 RBD was determined by BLI using a ForteBio Octet HTX (Sartorius). All binding steps were performed at 25° C. and at an orbital shaking speed of 1000 rpm. All reagents were formulated in PBSF (1×PBS with 0.1% w/v BSA). For ACE2 competition experiments, test antibodies (100 nM) were captured onto anti-human IgG capture (AHC) biosensors (Molecular Devices) to a sensor response of 1.0 nm-1.4 nm. IgG-loaded sensors were then soaked (20 min) in an irrelevant IgG1 solution (0.5 mg/ml) to block remaining Fc binding sites, followed by a 30 min incubation in PBSF. To assess any potential cross interactions between sensor-loaded IgG and ACE2, the IgG-loaded and blocked sensors were exposed (90 s) to a 300 nM ACE2 (Sino Biological, Cat #10108-H08H). Sensors were next allowed to baseline (60 s) before exposing (180 s) to recombinant SARS-COV-2 RBD (100 nM; Acro Biosystems, Cat #SPD-C52H3) and then exposed (180 s) to ACE2 (300 nM). Increased sensor responses following ACE2 exposure represented a non-ACE2-competitive binding profile, whereas antibodies showing unchanged sensor responses were designated as ACE2-competitive. Antibody competition with comparator antibodies (REGN10933, ADI-62113, COV2-2130, REGN10987, and S309) was performed using the same method as described above but with a different assay orientation: comparator antibodies were captured to anti-human IgG capture biosensors (Molecular Devices) and then exposed to antibodies of interest (300 nM) in solution.

### REFERENCES

1. T. Pilishvili et al., Effectiveness of mRNA Covid-19 Vaccine among U.S. Health Care Personnel. N Engl J Med 385, e90 (2021).
2. D. R. Feikin et al., Duration of effectiveness of vaccines against SARS-COV-2 infection and COVID-19 disease: results of a systematic review and meta-regression. Lancet 399, 924-944 (2022).
3. Y. Goldberg et al., Waning Immunity after the BNT162b2 Vaccine in Israel. N Engl J Med 385, e85 (2021).
4. K. B. Pouwels et al., Effect of Delta variant on viral burden and vaccine effectiveness against new SARS-COV-2 infections in the UK. Nat Med 27, 2127-2135 (2021).
5. L. J. Abu-Raddad, H. Chemaitelly, A. A. Butt, C.-V. National Study Group for, Effectiveness of the BNT162b2 Covid-19 Vaccine against the B.1.1.7 and B.1.351 Variants. N Engl J Med 385, 187-189 (2021).
6. N. Andrews et al., Covid-19 Vaccine Effectiveness against the Omicron (B.1.1.529) Variant. N Engl J Med, (2022).
7. H. Chemaitelly et al., Duration of mRNA vaccine protection against SARS-COV-2 Omicron BA.1 and BA.2 subvariants in Qatar. medRxiv, 2022.2003.2013.22272308 (2022).
8. W. Dejnirattisai et al., SARS-COV-2 Omicron-B.1.1.529 leads to widespread escape from neutralizing antibody responses. Cell, (2022).
9. L. Liu et al., Striking antibody evasion manifested by the Omicron variant of SARS-COV-2. Nature 602, 676-681 (2022).
10. A. Sokal et al., Immune escape of SARS-COV-2 Omicron variant from mRNA vaccination-elicited RBD-specific memory B cells. bioRxiv, 2021.2012.2021.473528 (2021).
11. S. Iketani et al., Antibody evasion properties of SARS-COV-2 Omicron sublineages. Nature, (2022).
12. V. Servellita et al., Neutralizing immunity in vaccine breakthrough infections from the SARS-COV-2 Omicron and Delta variants. medRxiv, 2022.2001.2025.22269794 (2022).
13. S. I. Richardson et al., SARS-COV-2 Omicron triggers cross-reactive neutralization and Fc effector functions in previously vaccinated, but not unvaccinated individuals. medRxiv, 2022.2002.2010.22270789 (2022).
14. M. S. Seaman et al., Vaccine Breakthrough Infection with the SARS-COV-2 Delta or Omicron (BA.1) Variant Leads to Distinct Profiles of Neutralizing Antibody Responses. medRxiv, 2022.2003.2002.22271731 (2022).
15. R. R. Goel et al., Efficient recall of Omicron-reactive B cell memory after a third dose of SARS-COV-2 mRNA vaccine. bioRxiv, 2022.2002.2020.481163 (2022).
16. F. Muecksch et al., Increased Potency and Breadth of SARS-COV-2 Neutralizing Antibodies After a Third mRNA Vaccine Dose. bioRxiv, (2022).
17. B. Briney, A. Inderbitzin, C. Joyce, D. R. Burton, Commonality despite exceptional diversity in the baseline human antibody repertoire. Nature 566, 393-397 (2019).
18. Z. Wang et al., mRNA vaccine-elicited antibodies to SARS-COV-2 and circulating variants. Nature 592, 616-622 (2021).
19. C. O. Barnes et al., SARS-COV-2 neutralizing antibody structures inform therapeutic strategies. Nature 588, 682-687 (2020).
20. M. Yuan et al., Structural and functional ramifications of antigenic drift in recent SARS-CoV-2 variants. Science 373, 818-823 (2021).
21. H. Liu et al., A recurring YYDRxG pattern in broadly neutralizing antibodies to a conserved site on SARS-COV-2, variants of concern, and related viruses. bioRxiv, 2021.2012.2015.472864 (2021).
22. M. Sakharkar et al., Prolonged evolution of the human B cell response to SARS-COV-2 infection. Sci Immunol 6, (2021).
23. Q. Zhang et al., Potent and protective IGHV3-53/3-66 public antibodies and their shared escape mutant on the spike of SARS-COV-2. Nat Commun 12, 4210 (2021).
24. H. A. McNamara et al., Antibody Feedback Limits the Expansion of B Cell Responses to Malaria Vaccination but Drives Diversification of the Humoral Response. Cell Host Microbe 28, 572-585 e577 (2020).
25. T. F. Rogers et al., Isolation of potent SARS-COV-2 neutralizing antibodies and protection from disease in a small animal model. Science 369, 956-963 (2020).
26. R. D. Gietz, R. H. Schiestl, High-efficiency yeast transformation using the LiAc/SS carrier DNA/PEG method. Nat Protoc 2, 31-34 (2007).

### Example 2: Evolution of Antibody Immunity Following Omicron BA.1 Breakthrough Infection

The emergence and global spread of the SARS-COV-2 Omicron BA.1 variant in late 2021 resulted in the largest surge in COVID-19 caseloads to date (1). While currently available COVID-19 vaccines induced high levels of protection against pre-Omicron variants, the extensive immune evasiveness of Omicron resulted in significantly reduced vaccine efficacy and durability following both primary and booster immunization (2-5). Moreover, antigenically drifted sub-lineages of Omicron (e.g. BA.2, BA.2.12.1, BA.4/5, BA.2.75, BA.2.75.2, and BA.4.6) continue to emerge and supplant prior sub-variants (4, 6, 7). The high prevalence of Omicron breakthrough infections led to the development and emergency use authorization of Omicron variant-based booster mRNA vaccines, despite limited safety and immunogenicity data in humans (2, 8). Thus, there is an urgent need to understand if and how secondary exposure to antigenically divergent variants, such as Omicron, shape SARS-COV-2-specific B cell memory.

The acute antibody response following Omicron BA. 1 breakthrough infection is dominated by re-activated memory B cells induced by mRNA vaccination (9-11). In support of these findings, preliminary data from clinical trials evaluating the immunogenicity of variant-based booster vaccines demonstrated that BA.1-containing mRNA vaccines induce a modest improvement in peak serum neutralizing responses compared with ancestral Wuhan-1 immunization (12). Although these studies provide evidence for “original antigenic sin” in the early B cell response following Omicron breakthrough infection, if and how this response evolves over time remains unclear. To address these questions, SARS-COV-2-specific serological and memory B responses were longitudinally profiled in mRNA-vaccinated donors up to six months following BA.1 breakthrough infection.

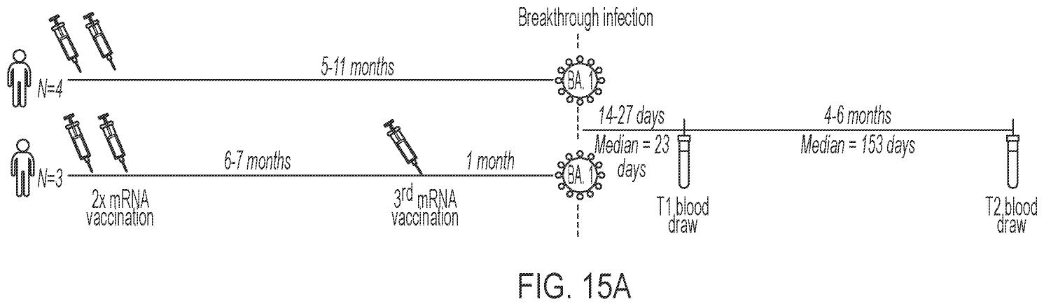

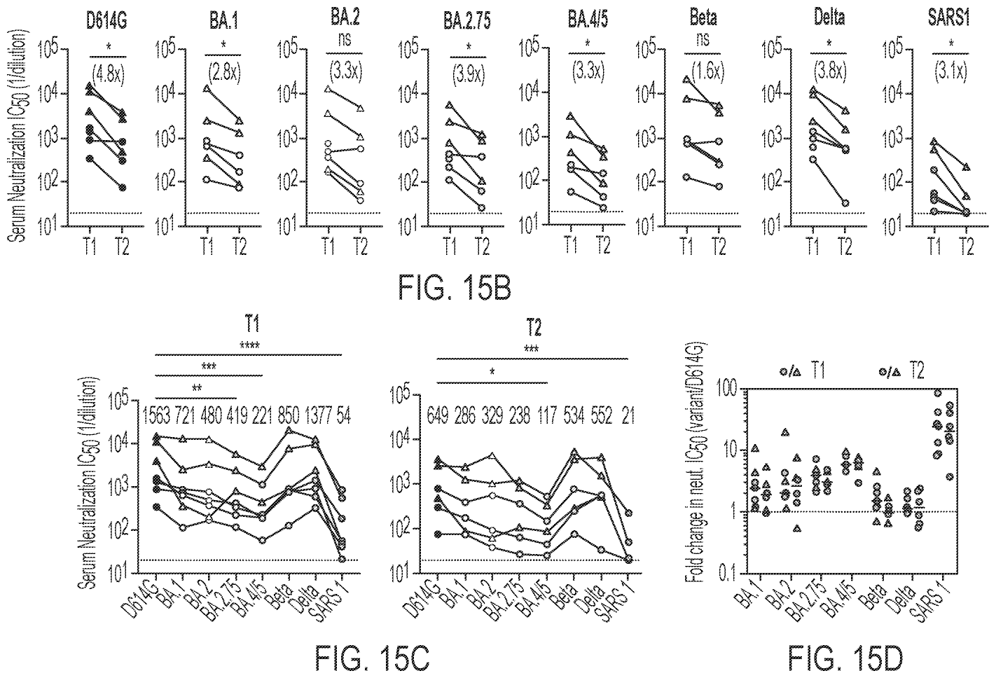

The antibody response to SARS-COV-2 was initially characterized in a cohort of seven mRNA-1273 vaccinated donors 14 to 27 days (median=23 days) after BA.1 breakthrough infection (9). To study the evolution of this response, blood samples were obtained from six of the seven participants at a follow-up appointment four to six months (median=153 days) post-infection (FIG. 15A, Table 7). Three of the six donors experienced infection after two-dose mRNA-1273 vaccination while the remaining three donors were infected after a third booster dose. None of the donors reported a second breakthrough infection between the two sample collection time points.

TABLE 7
Donor Characteristics.

| Donor ID | IML4041 | IML4042 | IML4043 | IML4044 | IML4045 | IML4054 | IML4055 |
|---|---|---|---|---|---|---|---|
| Age | 45 | 19 | 23 | 23 | 24 | 38 | 23 |
| Sex | F | F | M | F | F | F | F |
| Vaccination | 2x | 2x | 2x | 2x | 2x | 3x | 3x |
| History | BNT162b2 | BNT162b2 | BNT162b2 | BNT162b2 | BNT162b2, | mRNA-1273 | BNT162b2 |
| | | | | | 1x | | |
| | | | | | mRNA-1273 | | |
| Date of 2nd | 7 May 2021 | 22 Jul. 2021 | 23 May 2021 | 10 Feb. 2021 | 15 May 2021 | 5 May 2021 | 1 May 2021 |
| vaccination | | | | | | | |
| dose | | | | | | | |
| Date of 3rd | — | — | — | — | 20 Dec. 2021 | 11 Dec. 2021 | 9 Dec. 2021 |
| dose (if | | | | | | | |
| applicable) | | | | | | | |
| Date of | 31 Dec. 2021 | 4 Jan. 2022 | 30 Dec. 2021 | 2 Jan. 2022 | 6 Jan. 2022 | 19 Jan. 2022 | 6 Jan. 2022 |
| infection | | | | | | | |
| Days | 25 | 21 | 26 | 23 | 19 | 14 | 27 |
| between | | | | | | | |
| infection | | | | | | | |
| and first | | | | | | | |
| (T1) sample | | | | | | | |
| collection | | | | | | | |
| Days | N/A; | 170 | 139 | 139 | 168 | 122 | 168 |
| between | censored | | | | | | |
| infection | | | | | | | |
| and second | | | | | | | |
| (T2) sample | | | | | | | |
| collection | | | | | | | |

To evaluate serum neutralization breadth and potency, the plasma samples were tested for neutralizing activity against SARS-COV-2 D614G, emergent variants (BA.1, BA.2, BA.4/5, BA.2.75, Beta, and Delta), and the more evolutionarily divergent sarbecovirus SARS-COV, in a murine-leukemia virus (MLV)-based pseudovirus assay. Paired comparisons within each participant revealed that serum neutralizing titers against D614G declined by a median of 4.8-fold at 5- to 6-months post-infection relative to those observed within one-month post-infection (FIG. 15B). Correspondingly, lower serum neutralizing titers against Omicron sub-variants (2.8 to 3.9-fold, respectively), Beta (1.6-fold), Delta (3.8-fold), and SARS-COV (3.1-fold) at the 5- to 6-month time point relative to the early time point were observed (FIG. 15B). Despite this waning of neutralizing antibody titers over time, all of the donor sera displayed detectable neutralizing activity against all of the SARS-COV-2 variants tested at the 5-6 month time point (median titers ranging from 117 to 552) (FIG. 15C). Notably, titers remained within 3-fold of that observed for D614G for all variants except BA.4/5, which showed the greatest degree of escape from serum neutralizing antibodies (5.5-fold reduction from D614G), consistent with published serological studies (4, 5). Furthermore, the fold reduction in serum neutralizing titer for SARS-COV-2 VOCs relative to D614G remained similar at both time points, suggesting maintained serum neutralization breadth over time (FIG. 15D). Minimal cross-neutralizing activity against SARS-COV (median titer=21) was observed in all donors, suggesting that serum neutralization breadth remained limited to SARS-COV-2 variants (FIG. 15C). It was concluded that serum neutralizing titers wane over the course 6-months following Omicron BA.1 breakthrough infection but nevertheless remain at detectable levels across a diverse range of SARS-COV-2 variants through 6 months.

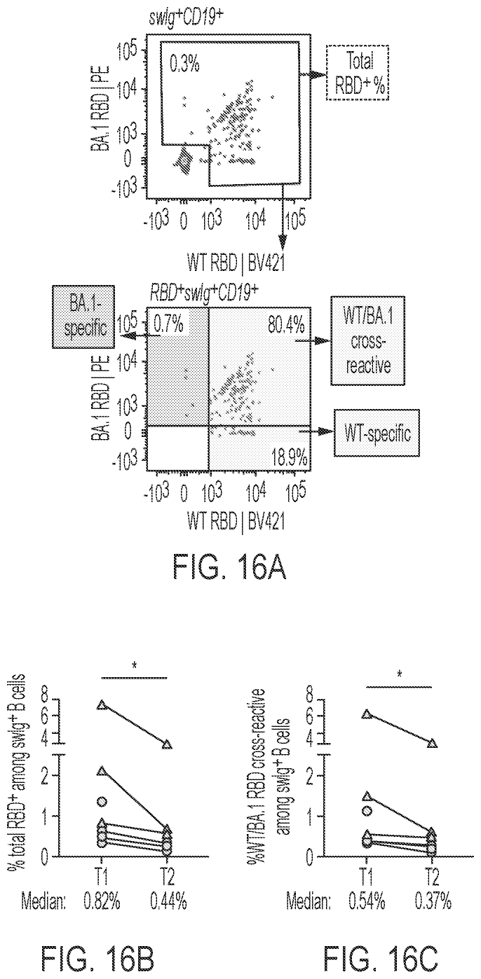

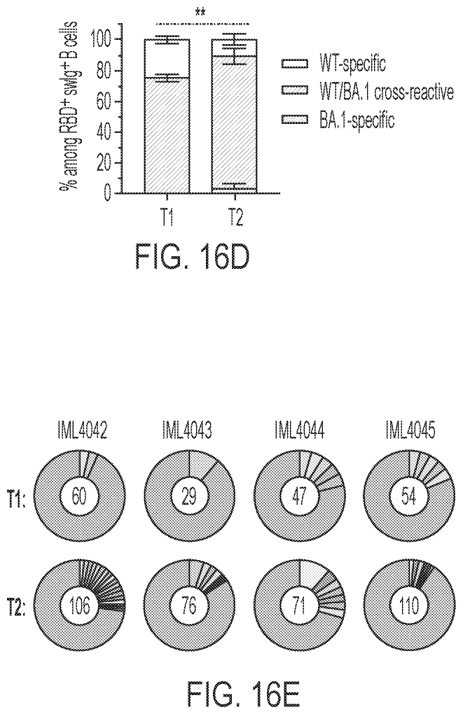

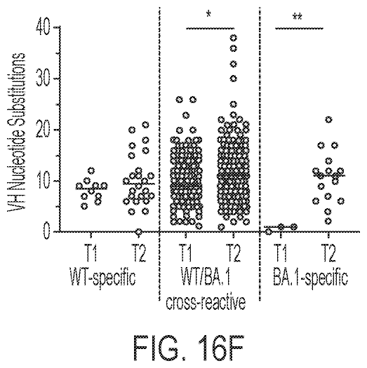

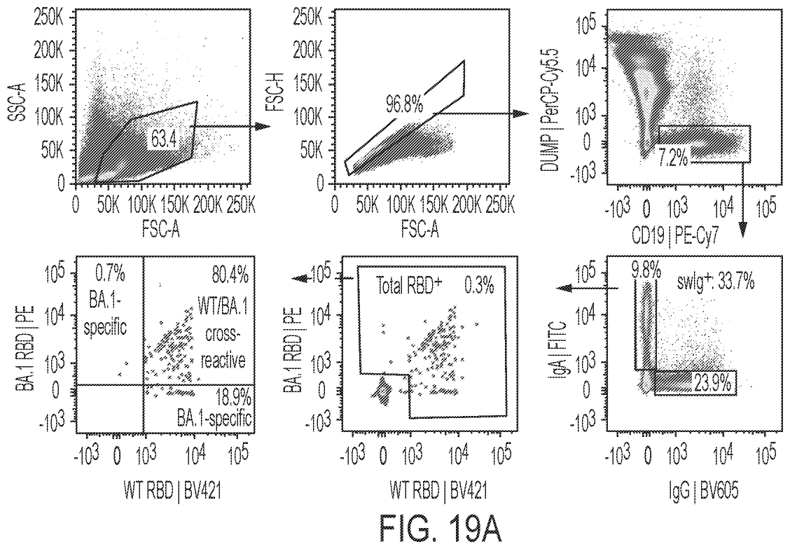

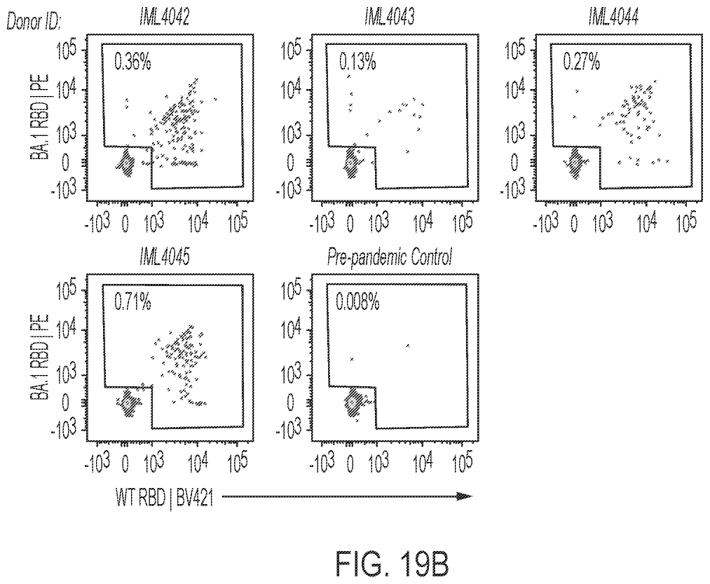

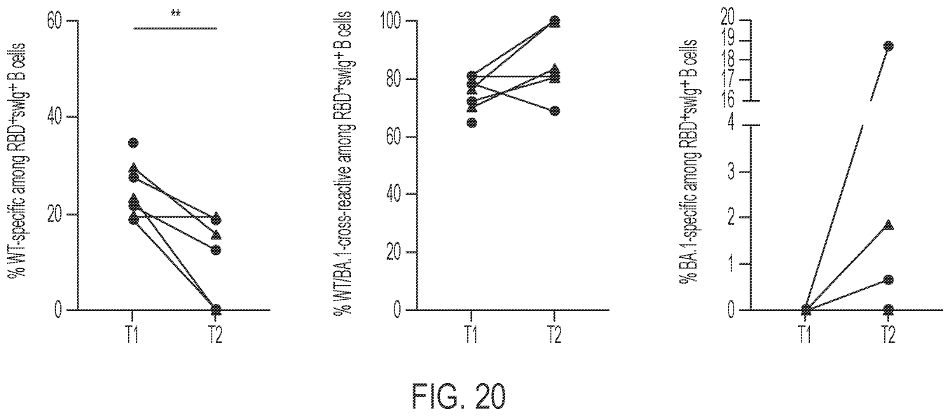

Next, the magnitude and cross-reactivity of the antigen-specific B cell response were assessed via flow cytometric enumeration of B cells stained with differentially labeled wildtype (Wuhan-1; WT) and BA.1 RBD tetramers (FIG. 16A, FIG. 19A). At the 5-6-month time point, total RBD-reactive B cells (WT and/or BA.1-reactive) and WT/BA.1 cross-reactive B cells comprised a median of 0.44% (ranging 0.12-2.53%) and 0.37% (ranging 0.12-2.53%) of class-switched (IgG⁺ or IgA⁺) B cells, respectively (FIG. 16B, FIG. 16C, FIG. 19A). Thus, 86% (ranging 69-100%) of all RBD+ class-switched B cells at 5-6 months post-infection displayed BA.1/WT cross-reactivity, compared with 75% at 1-month post-infection (ranging 65-81%) (FIG. 16D, FIG. 20). Correspondingly, WT-specific B cells decreased from 25% of all RBD+ class-switched B cells at 1 month to 11% at 5-6 months (FIG. 16D, FIG. 20). Consistent with the waning of serum neutralizing titers over time, a modest but significant decline (1.1 to 3.7-fold) in the frequencies of WT/BA.1 cross-reactive B cells at 5-6 months relative to the 1-month time point was also observed (FIG. 16C). At the late time point, the emergence of a BA.1-specific B cell population (average=3% of class-switched B cells) was detected in 3 of the 6 individuals, although the magnitude of this response varied widely among individuals (ranging from 1-18%) (FIG. 16D, FIG. 20). In summary, Omicron BA.1 breakthrough infection induces a WT/BA.1 cross-reactive B cell response at early time points post-infection and this response only modestly declines over the course of 6 months.

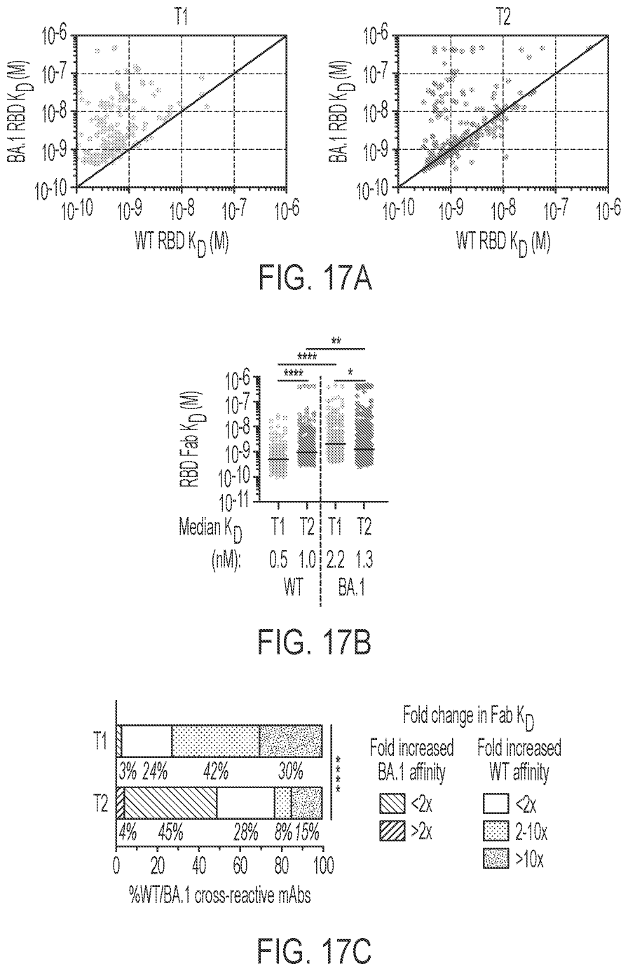

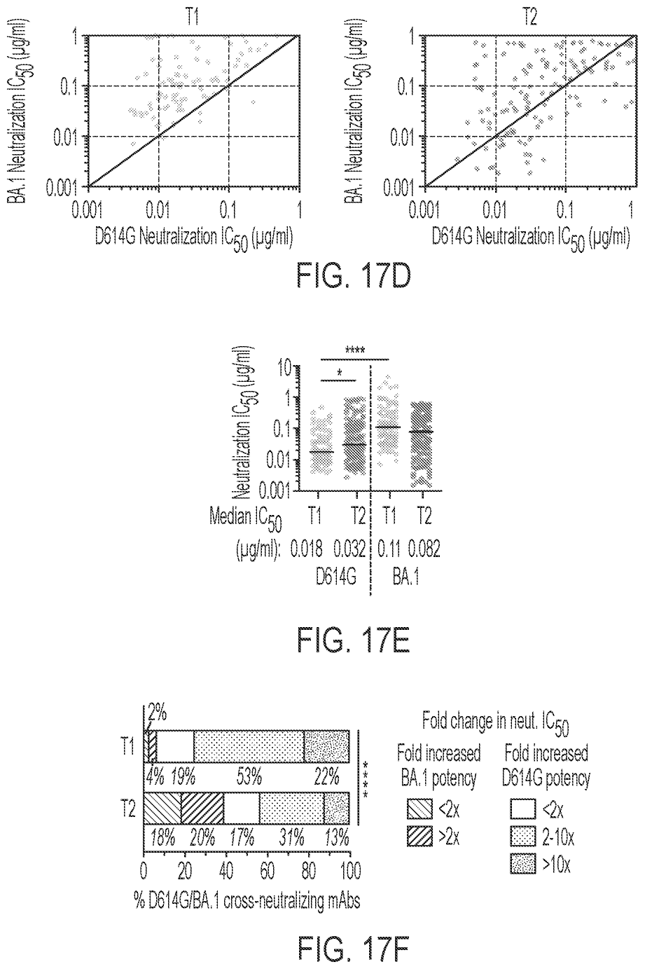

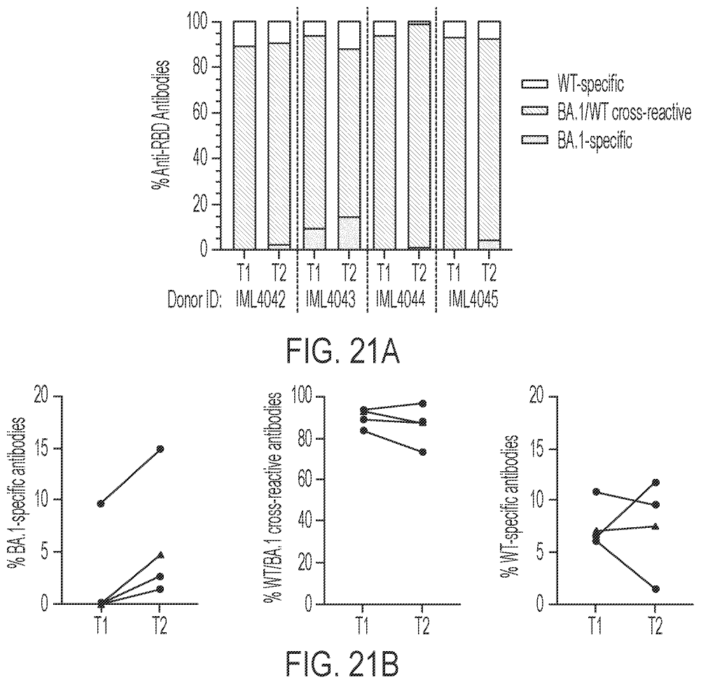

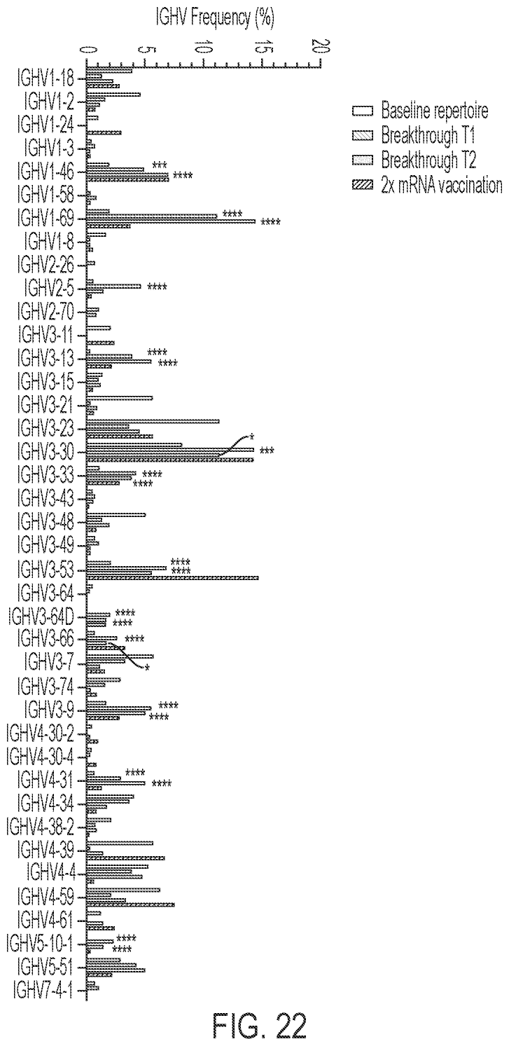

To compare the molecular characteristics of antibodies isolated at early and late time points following BA.1 breakthrough infection, 71 to 110 class-switched RBD-reactive B cells were single-cell sorted from four of the five previously studied donors (donors IML4042, IML4043, IML4044, IML4045) at 139 to 170 days after breakthrough infection and expressed a total of 363 natively paired antibodies as full-length IgGs (FIG. 19B) (9). Similar to the antibodies characterized from the acute time point, the newly isolated antibodies primarily recognized both WT and BA.1 RBD antigens (73-97%), exhibited a high degree of clonal diversity, and displayed preferential usage of certain VH germline genes (IGHV1-46, 1-69, 3-13, 3-53, 3-66, 3-9, and 4-31 germline genes at both time points) (FIG. 16E, FIGS. 21A-B and FIG. 22). The level of SHM in the cross-reactive antibodies increased from a median of 9 VH nucleotide substitutions at 1-month to 11 VH nucleotide substitutions by 5-6 months, potentially suggesting affinity maturation in secondary germinal centers (FIG. 16F). Consistent with their higher levels of SHM, the antibodies isolated at 5-6 months displayed 1.7-fold improved binding to BA.1 (median KD=1.3 nM) and 2-fold reduced binding affinity to the WT RBD (median K_{D}=1.0 nM) relative to early antibodies, suggesting maturation towards Omicron BA.1 at the expense of WT affinity (FIG. 17A and FIG. 17B). These changes in binding recognition resulted in the late antibodies showing more balanced affinity profiles compared to the early antibodies (FIG. 17B). For example, the majority of antibodies (73%) isolated at the late time point exhibited WT and BA.1 RBD affinities within two-fold of each other compared to only 24% of early antibodies (FIG. 17C).

To determine whether the improvement in binding affinity for BA.1 translated into enhanced neutralization potency, the antibodies were assessed for neutralizing activity against WT and BA.1 using a pseudovirus assay. Fifty-one percent and 42% of WT/BA.1 cross-binding antibodies isolated from the 1-month and 5-6-month time point, respectively, cross-neutralized D614G and BA.1 with IC₅₀s<2 μg/ml. Overall, the neutralizing antibodies displayed approximately 2-fold lower potency against D614G at the late time point relative to the acute time point, consistent with the observed reduction in WT RBD affinity over time (FIG. 17D and FIG. 17E). Furthermore, consistent with the improvement in BA.1 RBD affinity over time, the antibodies isolated at 6 months trended towards more potent neutralization of BA.1 relative to antibodies isolated at 1 month (FIG. 17E). Forty-one percent of the neutralizing antibodies isolated at 6 months exhibited more potent activity against BA.1 relative to D614G, compared to only 7% of the acute neutralizing antibodies (FIG. 17F). In summary, cross-reactive antibody responses induced following BA.1 breakthrough infection evolve toward increased BA.1 affinity and neutralization potency for at least 6 months post-infection.

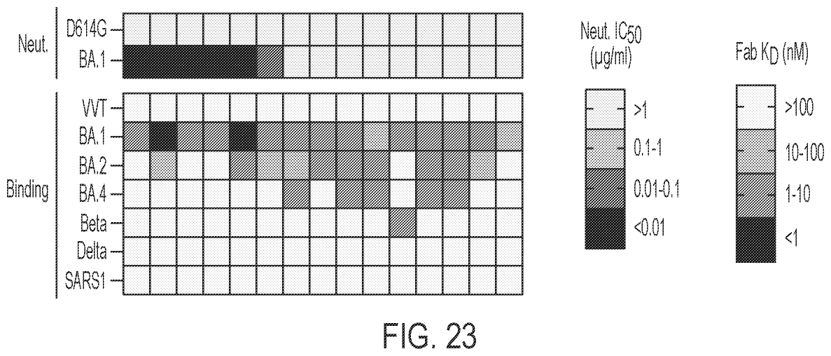

Although the vast majority of antibodies isolated at the 5-6-month time point displayed WT/BA.1 cross-reactive binding, a limited number of BA.1-specific antibodies were identified in all four donors, comprising 1% to 15% of total RBD-specific antibodies (median=4%) (FIGS. 21A-B). In contrast, BA.1-specific antibodies were only detected in a single donor at the acute time point (FIGS. 21A-B). Furthermore, unlike the BA.1-specific antibodies isolated at the early time point, which lacked somatic mutations, the BA.1-specific antibodies identified at 5-6 months displayed SHM levels similar to those of cross-reactive antibodies (median=11 VH nucleotide substitutions) (FIG. 16F). Forty percent of BA.1-specific antibodies isolated at the late time point neutralized BA.1, with IC₅₀s ranging from 0.002 to 0.089 μg/ml, and none of the antibodies displayed detectable neutralizing activity against D614G (FIG. 23). Thus, BA.1 breakthrough infection induces a limited and delayed de novo Omicron-specific B cell response that undergoes affinity maturation over time.

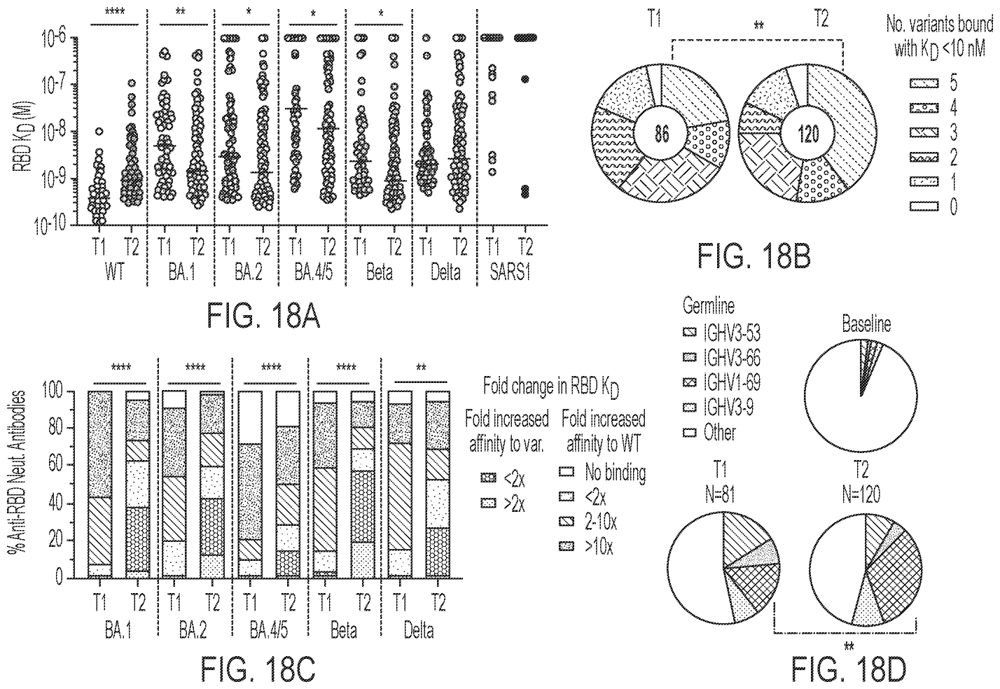

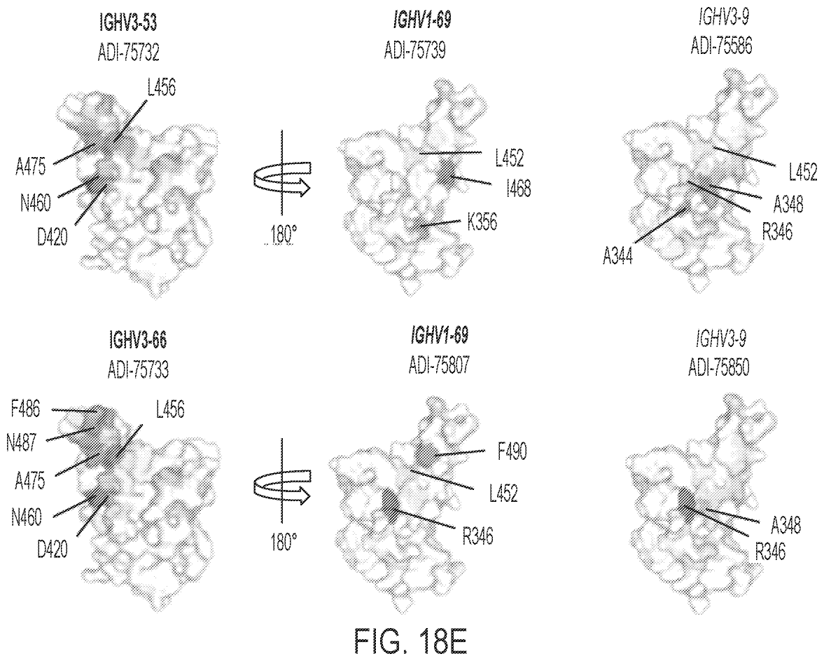

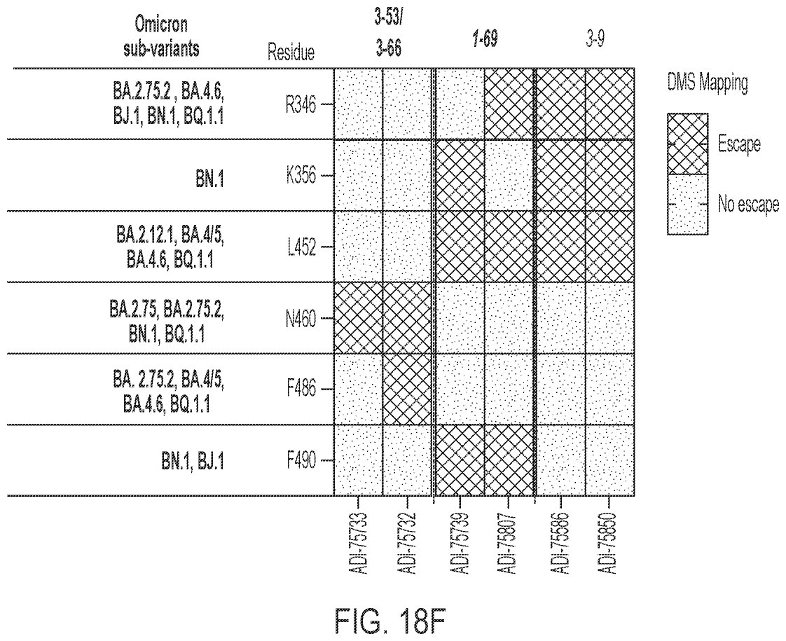

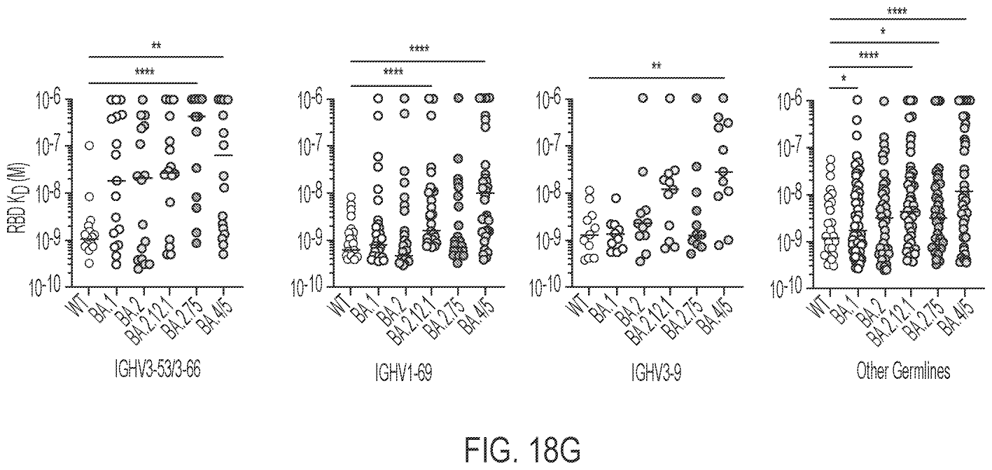

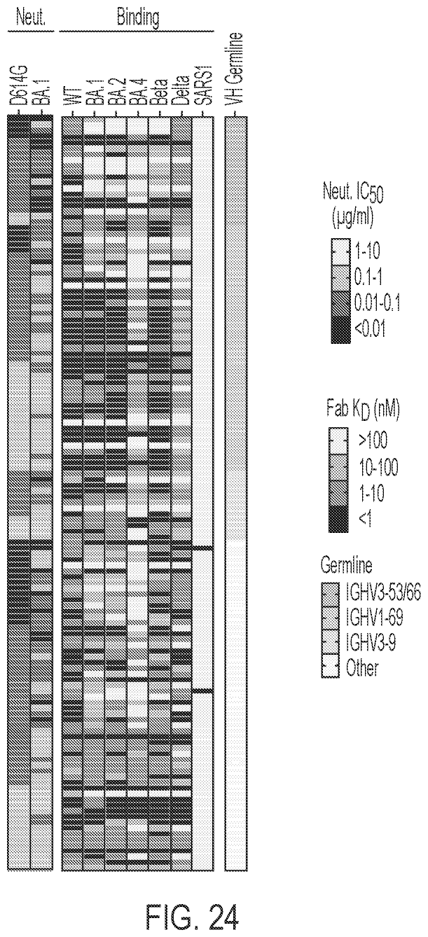

To further explore the breadth of both WT/BA. 1 cross-reactive and BA.1-specific neutralizing antibodies, their binding reactivities were evaluated with a panel of recombinant RBDs encoding mutations present in SARS-COV-2 variants BA.2, BA.4/5, Beta, and Delta, and the more antigenically divergent SARS-COV. D614G/BA.1 cross-neutralizing antibodies displayed 2.4-fold reduced affinity for the WT RBD and 3.4-fold improved affinity for the BA.1 RBD relative to early neutralizing antibodies, consistent with the pattern observed for all WT/BA.1 cross-binding antibodies (FIG. 18A, FIGS. 17A-C). Furthermore, the WT/BA.1 cross-reactive antibodies isolated at 6 months broadly recognized other SARS-COV-2 variants, except for BA.4/5, which was associated with a ≥5-fold loss in affinity for 57% (68/120) of the WT/BA.1 neutralizing antibodies (FIG. 18A, FIG. 24). Importantly, the 5-6-month antibodies displayed higher affinity binding to all Omicron sub-variants and Beta relative to the early antibodies, suggesting that the increased affinity to BA.1 also improved breadth of reactivity against other variants (FIG. 18A). In support of this finding, a significantly higher proportion (40%) of neutralizing antibodies isolated at 6 months displayed high affinity (K_{D}<10 nM) binding to all five variants tested compared with early antibodies (22%) (FIG. 18B). Furthermore, antibodies isolated at the late time point displayed smaller differences in binding affinity against BA.1, BA.2, BA.4/5 and the early Beta and Delta variants relative to early antibodies (FIG. 18C). In contrast to the WT/BA.1 cross-reactive antibodies, the BA.1-specific neutralizing antibodies displayed limited breadth, with only 50% of these antibodies maintaining binding to BA.2 and none of the antibodies showing reactivity with WT, BA.4/5, Beta, or Delta (FIG. 23). It was concluded that BA.1 breakthrough infection results in an overall broadening of the anti-SARS-COV-2 neutralizing antibody repertoire.

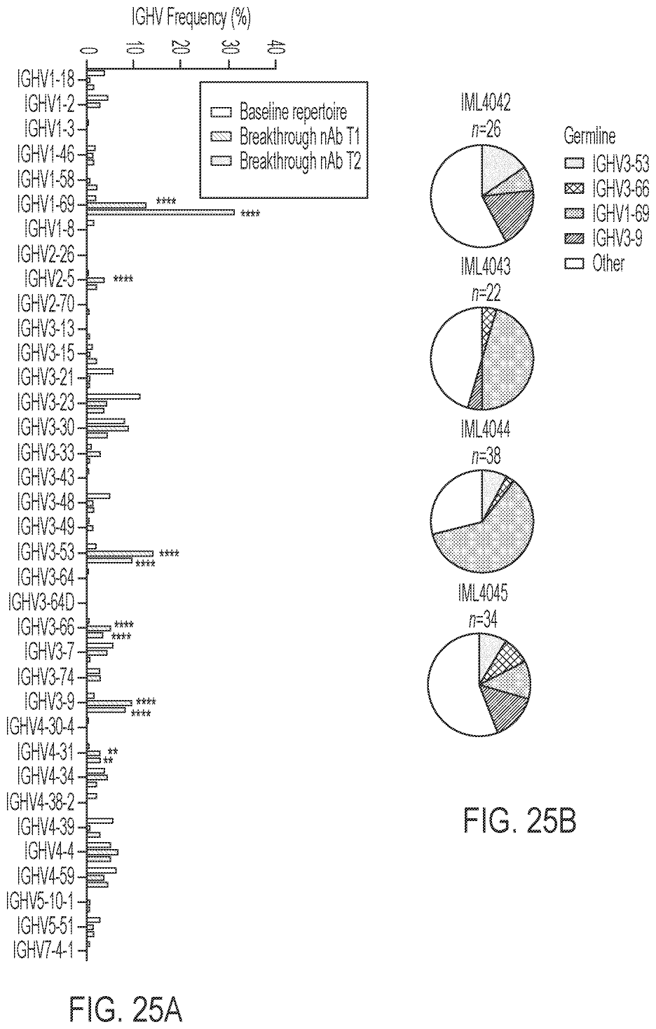

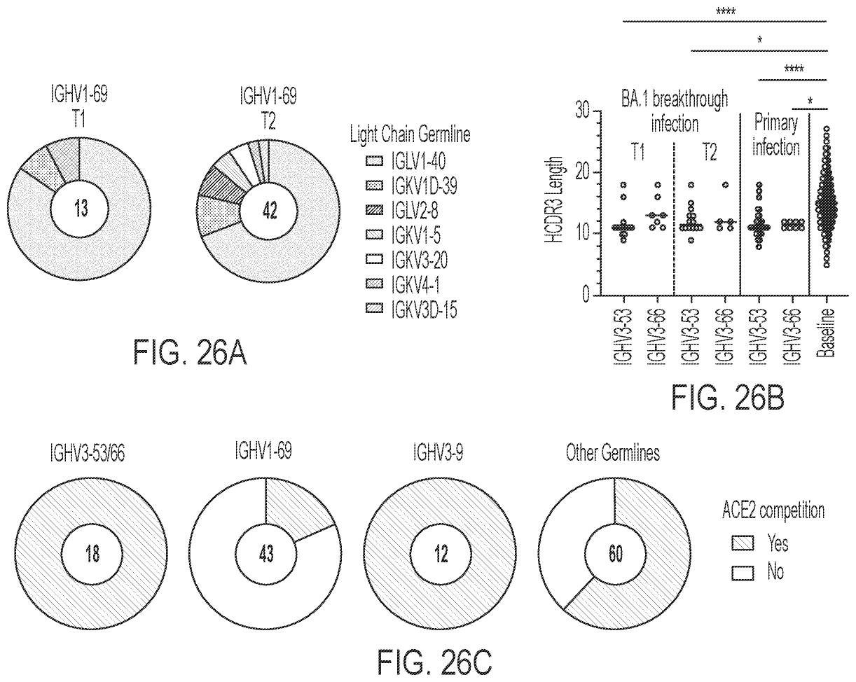

Among neutralizing antibodies isolated at both time points, significant over-representation of four IGHV germline genes (IGHV1-69, IGHV3-53/3-66, and IGHV3-9) were observed (9) (FIG. 25A). At the 5-6-month time point, over half (54%) of the neutralizing antibodies were encoded by one of these four germlines, with one-third of these antibodies utilizing IGHV1-69 (FIG. 18D, FIGS. 25A-B). It was previously found that BA.1-neutralizing IGHV1-69 antibodies isolated from the early time point preferentially paired with the light chain germline IGLV1-40 and targeted an antigenic site overlapping that of the class 3 antibody COV2-2130 and non-overlapping with the ACE2 binding site (9). Similarly, 69% of IGHV1-69 antibodies isolated at 5-6 months paired with the IGLV1-40 germline and the majority (80%) failed to compete with ACE2 for binding (FIGS. 26A and 26C). Likewise, >90% of IGHV3-9 antibodies identified from both time points recognized a non-ACE2-competitive binding site, although unlike IGHV1-69 antibodies, IGHV3-9 antibodies recognize an epitope overlapping S309 and REGN10987 as well as COV2-2130, suggesting a distinct mode of binding from IGHV1-69 antibodies (FIG. 26C) (9). Lastly, IGHV3-53/66 antibodies isolated from both time points were characterized by short HCDR3s (median=11 to 12 nucleotide substitutions) compared with baseline HCDR3 lengths (median=15 substitutions) and displayed competitive binding with the ACE2 receptor (FIGS. 26B and 26C). Thus, convergent antibody classes dominated the neutralizing antibody response at both early and late time points following BA.1 breakthrough infection, suggesting little to no change in B cell immunodominance hierarchy over time.

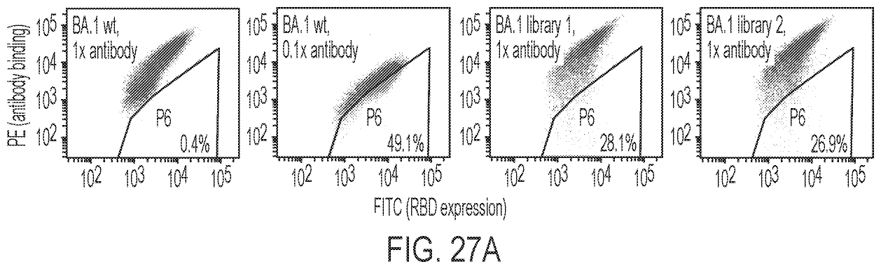

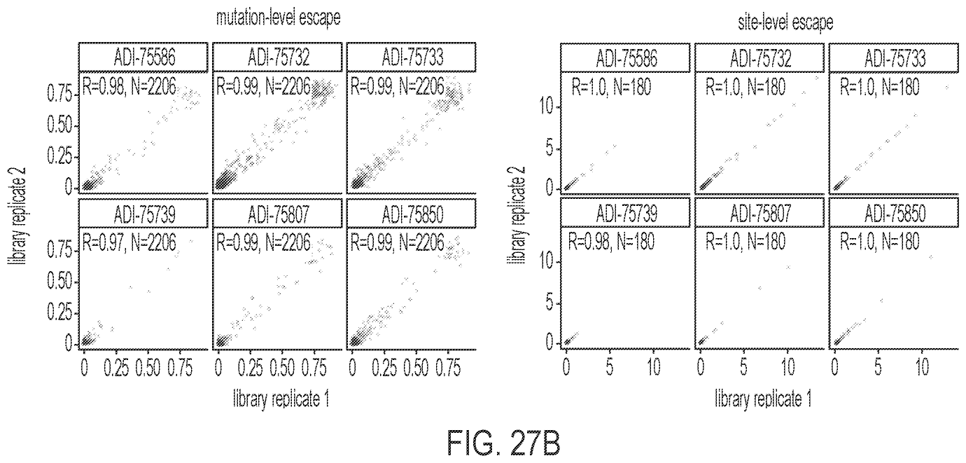

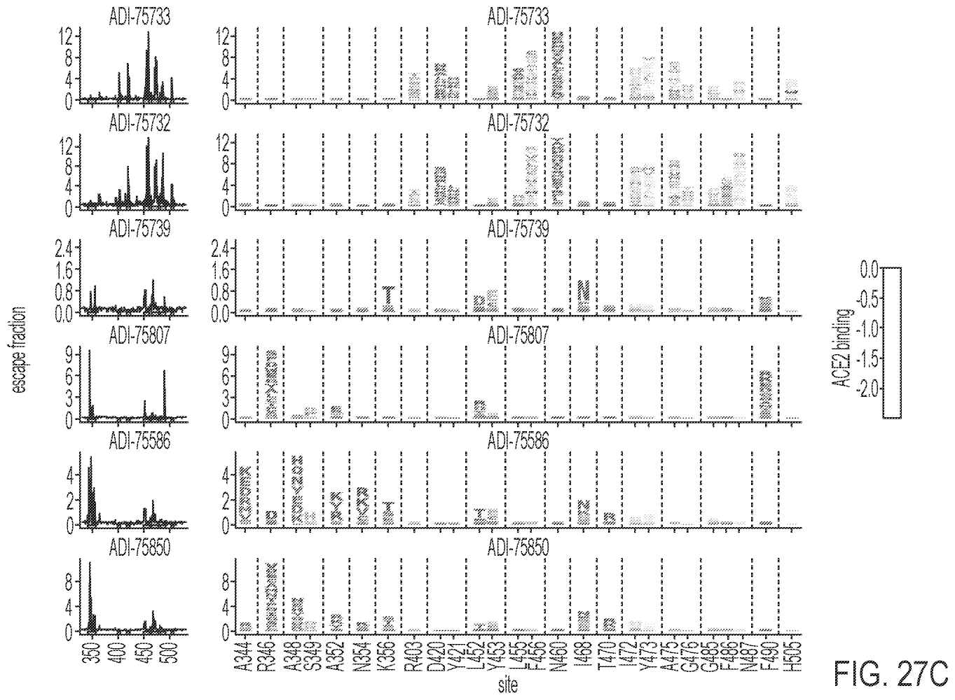

Given the dominance of these public clonotypes in BA.1 breakthrough infection donors, their escape mutations in the BA.1 background were determined. One to two antibodies belonging to each convergent germline were randomly selected and deep mutational scanning (DMS) analysis was performed using a library encoding all possible amino acid substitutions from BA.1 (FIG. 27A) (13). Antibodies encoded by IGHV3-53 (ADI-75733) and IGHV3-66 (ADI-75732) displayed similar escape profiles, consistent with their shared sequence features and competitive binding profiles (FIG. 18E and FIG. 27C) (9). RBD positions N460 and F486, which are mutated in emergent variants (N460K in B.2.75, BA.2.75.2, BN.1, and BQ.1; F486S in BA.2.75.2; and F486V in BA.4/5, BA.4.6, and BQ.1.1), were associated with binding escape from IGHV3-53/66 antibodies (FIG. 18F and FIG. 27C). IGHV1-69 and IGHV3-9 antibodies both showed reduced binding to RBDs incorporating mutations at positions 344-349, 356, 452-453, 468, and 490. Notably, residues R346, K356, L452, and F490 are mutated across evolutionarily diverse Omicron sub-lineages, including BA.4.6 (R346T, L452R), BA.4/5 (L452R), BA.2.12.1 (L452Q), BJ.1 (R346T, F490V), BN.1 (R346T, K356T, F490S), and BQ.1.1 (R346T, L452R) (FIG. 18F and FIG. 27C) (6). Consistent with these escape profiles, IGHV1-69 and IGHV3-9 class antibodies displayed reduced binding to BA.2.12.1 and BA.4/5 relative to early Omicron variants, likely due to the unique L452Q/R mutations present in these variants compared with BA.1 and BA.2 (FIG. 18G). Consistent with DMS-based predictions, both BA.2.75 and BA.4/5 RBDs displayed increased binding resistance to IGHV3-53/66 antibodies (FIGS. 18F and 18G). Thus, convergent D614G/BA.1 cross-neutralizing antibodies recognize epitopes commonly mutated in recently emerging Omicron sub-variants, providing a molecular explanation for the high degree of antigenic convergence observed in recent Omicron sub-variant evolution and their increased level of immune evasion relative to BA.1.

In summary, BA.1 breakthrough infection in mRNA-vaccinated individuals induces broadly neutralizing serological and MBC responses that persist for at least six months after infection, supporting real-world studies showing that BA.1 breakthrough infection provides protection against symptomatic BA.1, BA.2, and BA.5 infection for at least 5-6 months (14-16). Furthermore, although the acute B cell response following breakthrough infection is primarily mediated by recall of cross-reactive vaccine-induced MBCs, these MBC clones accumulate somatic mutations and evolve increased breadth and potency for at least 6 months following infection. Although this enhanced neutralization breadth and potency was not reflected in the serum antibody response, it is possible that a second heterologous exposure may broaden the serological repertoire by activating these affinity matured MBCs, akin to the improved serum neutralization breadth observed following mRNA booster vaccination (17, 18). Nevertheless, these data indicate that infection or vaccination with antigenically divergent SARS-COV-2 variants may provide long-term benefits by broadening pre-existing anti-SARS-COV-2 B cell memory.

Finally, it was found that convergent classes of neutralizing antibodies dominated the BA.1 breakthrough response at both early and late time points, reminiscent of the antibody response elicited following primarily infection or vaccination with early ancestral SARS-COV-2 strains (19-21). The sustained prevalence of public clones that target residues frequently mutated in emerging Omicron subvariants offers a molecular explanation for the continued antigenic drift of Omicron. Thus, in contrast to current approaches to the design of universal vaccines for highly antigenically variable viruses, such as HIV and influenza, which aim to focus the neutralizing response on a limited number of relatively conserved epitopes, the development of “variant-proof” COVID-19 vaccines may require a different strategy: engineering of spike-based immunogens that induce neutralizing antibody responses to a limited set of somewhat variable, non-overlapping epitopes, with the goal of limiting convergent immune pressure (22-24).

### Materials and Methods

### Human Subjects and Blood Sample Collection.

Seven BA.1 breakthrough infected participants were recruited to participate in this study with informed consent under the healthy donor protocol D10083, Immune Monitoring Core (DartLab) Laboratory at Dartmouth-Hitchcock Hospital, as previously described (9). Briefly, participants experienced breakthrough infection after two- or three-dose mRNA vaccination (BNT162b2 and/or mRNA-1273). Venous blood was collected at two time points, an early visit at 14 to 27 days (T1) and a late visit 139 to 170 days (T2) after their first SARS-COV-2 test. Participants had no documented history of SARS-COV-2 infection prior to vaccination or between the two blood draw time points. Clinical and demographic characteristics of breakthrough infection donors are shown in Table 7. Plasma and peripheral blood mononuclear cell (PBMC) samples were isolated using a Ficoll 1077 (Sigma) gradient, as previously described (9).

### Plasmid Design and Construction.

Plasmids expressing spike proteins of SARS-COV-2 variants and SARS-COV were ordered as gene block fragments (IDT) and cloned into a mammalian expression vector for MLV-based pseudovirus production as previously described (26). All SARS-COV-2 variant spikes and the SARS-CoV spike were C-terminally truncated by 19-amino acids or 28-amino acids, respectively, to increase infectious titers. The SARS-COV S sequence was retrieved from ENA (AAP13441). SARS-COV-2 variants contain the following mutations from the Wuhan-Hu-1 sequence (Genbank: NC_045512.2):
D614G: D614G.
Beta: D80A, D215G, Δ242-244, K417N, E484K, N501Y, D614G, A701V
Delta: T19R, G142D, Δ156-157, R158G, L452R, T478K, D614G, P681R, D950N
BA.1: A67V, Δ69-70, T95I, G142D/Δ143-145, Δ211/L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F
BA.2: T19I, L24S, Δ25-27, G142D, V213G, G339D, S371F, S373P, S375F, T376A, D405N, R408S, K417N, N440K, S477N, T478K, E484A, Q493R, Q498R, N501Y, Y505H, D614G, H655Y, N679K, P681H, N764K, D796Y, Q954H, N969K
BA.4/5: T19I, L24S, Δ25-27, Δ69-70, G142D, V213G, G339D, S371F, S373P, S375F, T376A, D405N, R408S, K417N, N440K, L452R, S477N, T478K, E484A, F486V, Q498R, N501Y, Y505H, D614G, H655Y, N679K, P681H, N764K, D796Y, Q954H, N969K
BA.2.75: T19I, L24S, Δ25-27, G142D, K147E, W152R, F157L, 1210V, V213G, G339H, G257S, S371F, S373P, S375F, T376A, D405N, R408S, K417N, N440K, G446S, N460K, S477N, T478K, E484A, Q498R, N501Y, Y505H, D614G, H655Y, N679K, P681H, N764K, D796Y, Q954H, N969K

### SARS-COV-2 Pseudovirus Generation.

Single-cycle infectious MLVs pseudotyped with spike proteins of SARS-COV-2 variants and SARS-COV were generated as previously described (26). Briefly, HEK293T cells were seeded at a density of 0.5 million cells/ml in 6-well tissue culture plates and the next day, transfected using Lipofectamine 2000 (ThermoFisher Scientific) with the following plasmids: 1) 0.5 μg per well of pCDNA3.3 encoding SARS-COV-2 spike with a 19-amino acid truncation at the C-terminus, 2) 2 μg per well of MLV-based luciferase reporter gene plasmid (Vector Builder), and 3) 2 μg per well of MLV gag/pol (Vector Builder). MLV particles were harvested 48 h post-transfection, aliquoted, and stored at −80° C. for neutralization assays.

### Pseudovirus Neutralization Assay.

MLV pseudovirus neutralization assays for serum and monoclonal antibodies were performed as previously described (9). Briefly, 56° C. heat-inactivated sera or antibodies were serially diluted in 50 μl MEM/EBSS media supplemented with 10% fetal bovine serum (FBS) and incubated with 50 μl of MLV viral stock for 1 h at 37° C. Following incubation, antibody-virus mixtures were added to previously seeded HeLa-hACE2 reporter cells (BPS Bioscience Cat #79958). Infection was allowed to occur for 48 h at 37° C. Infection was measured by lysing cells with Luciferase Cell Culture Lysis reagent (Promega) and detecting luciferase activity using the Luciferase Assay System (Promega) following manufacturer's protocols. Infectivity was as quantified by relative luminescence units (RLUs) and the percentage neutralization was calculated as 100*(1−[RLUsample−RLUbackground]/[RLUisotype control mAb−RLUbackground]). Neutralization IC₅₀ was interpolated from curves fitted using four-parameter non-linear regression in GraphPad Prism (version 9.3.1).

### FACS Analysis of SARS-COV-2 S-Specific B Cell Responses.

Antigen-specific B cells were detected using recombinant biotinylated antigens tetramerized with fluorophore-conjugated streptavidin (SA), as previously described (9). Briefly, Avitag biotinylated WT RBD (Acro Biosystems, Cat #SPD-C82E8) and Avitag biotinylated BA.1 RBD (Acro Biosystems, Cat #SPD-C82E4) were mixed in 4:1 molar ratios with SA-BV421 (BioLegend) and SA-phycoerythrin (PE; Invitrogen), respectively, and allowed to incubate for 20 min on ice. Unbound SA sites were subsequently quenched using 5 μl of 2 μM Pierce biotin (ThermoFisher Scientific). Approximately 10 million PBMCs were stained with tetramerized RBDs (25 nM each); anti-human antibodies anti-CD19 (PE-Cy7; Biolegend), anti-CD3 (PerCP-Cy5.5; Biolegend), anti-CD8 (PerCP-Cy5.5; Biolegend), anti-CD14 (PerCP-Cy5.5; Invitrogen), and anti-CD16 (PerCP-Cy5.5; Biolegend); and 50 μl Brilliant Stain Buffer (BD BioSciences) diluted in FACS buffer (2% BSA/1 mM EDTA in 1×PBS). 200 μl of staining reagents were added to each PBMC sample and incubated for 15 min on ice. After one wash with FACS buffer, cells were stained in a mixture of propidium iodide and anti-human antibodies anti-IgG (BV605; BD Biosciences), anti-IgA (FITC; Abcam), anti-CD27 (BV510; BD Biosciences), and anti-CD71 (APC-Cy7; Biolegend). Following 15 min of incubation on ice, cells were washed two times with FACS buffer and analyzed using a BD FACS Aria II (BD BioSciences).

For sorting of RBD-specific, class-switched B cells, PBMCs that react with either WT and/or BA.1 RBD tetramers among CD19⁺CD3⁻CD8⁻CD14⁻CD16⁻PI⁻ and IgG⁺ or IgA⁺ cells were single-cell index sorted into 96-well polystyrene microplates (Corning) containing 20 μl lysis buffer per well [5 μl of 5× first strand SSIV cDNA buffer (Invitrogen), 1.25 μl dithiothreitol (Invitrogen), 0.625 μl of NP-40 (Thermo Scientific), 0.25 μl RNaseOUT (Invitrogen), and 12.8 μl dH2O]. Plates briefly centrifuged and then frozen at −80° C. before PCR amplification.

### Amplification and Cloning of Antibody Variable Genes.

Antibody variable gene fragments (VH, Vk, Vλ) were amplified by RT-PCR as described previously (27). Briefly, cDNA was synthesized using randomized hexamers and SuperScript IV enzyme (ThermoFisher Scientific). cDNA was subsequently amplified by two rounds of nested PCRs, with the second cycle of nested PCR adding 40 base pairs of flanking DNA homologous to restriction enzyme-digested S. cerevisiae expression vectors to enable homologous recombination during transformation. PCR-amplified variable gene DNA was mixed with expression vectors and chemically transformed into competent yeast cells via the lithium acetate method (28). Yeast were plated on selective amino acid drop-out agar plates and individual yeast colonies were picked for sequencing and recombinant antibody expression.

### Expression and Purification of IgG and Fab Molecules.

Antibodies were expressed as human IgG1 via S. cerevisiae cultures, as described previously (27). Briefly, yeast cells were grown in culture for 6 days for antibody production, before collecting IgG-containing supernatant by centrifugation. IgGs were subsequently purified by protein A-affinity chromatography and eluted using 200 mM acetic acid/50 mM NaCl (pH 3.5). The pH was then neutralized using ⅛^{th} volume of 2 M Hepes (pH 8.0). Fab fragments were cleaved from full-length IgG by incubating with papain for 2 h at 30° C. before terminating the reaction using iodoacetamide. Fab fragments were purified from the mixture of digested antibody Fab ad Fc fragments using a two-step chromatography system: 1) Protein A agarose was used to remove Fc fragments and undigested IgG, and 2) Fabs in the flow-through were further purified using CaptureSelect™ IgG-CH1 affinity resin (ThermoFisher Scientific) and eluted from the column using 200 mM acetic acid/50 mM NaCl (pH 3.5). Fab solutions were pH-neutralized using ⅛th volume 2 M Hepes (pH 8.0).

### Binding Affinity Measurements by Biolayer Interferometry.

Antibody binding kinetics were measured by biolayer interferometry (BLI) using a FortéBio Octet HTX instrument (Sartorius). All steps were performed at 25° C. and at an orbital shaking speed of 1000 rpm, and all reagents were formulated in PBSF buffer (PBS with 0.1% w/v BSA). To measure monovalent binding affinities against SARS-COV-2 RBD variants and SARS-COV S, recombinant RBDs of SARS-COV-2 WT (Acro Biosystems, Cat #SPD-C52H3), Beta (Acro Biosystems, Cat #SPD-C52Hp), Delta (Acro Biosystems, Cat #SPD-C52Hh), BA.1 (Acro Biosystems, Cat #SPD-C522f), BA.2 (Acro Biosystems, Cat #SPD-C522g), BA.4/5 (Acro Biosystems, Cat #SPD-C522r), and SARS-CoV (Sino Biological, Cat #40150-V08B2) were biotinylated using EZ-Link™ Sulfo-NHS-LC-Biotin (Thermo Scientific) following manufacturer's recommendations to achieve an average of 4 biotins per RBD molecule. Biotinylated antigens were diluted (100 nM) in PBSF and loaded onto streptavidin biosensors (Sartorius) to a sensor response of 1.0-1.2 nm and then allowed to equilibrate in PBSF for a minimum of 30 min. After a 60 s baseline step in PBSF, antigen-loaded sensors were exposed (180 s) to 100 nM Fab and then dipped (420 s) into PBSF to measure any dissociation of the antigen from the biosensor surface. Fab binding data with detectable binding responses (>0.1 nm) were aligned, inter-step corrected (to the association step) and fit to a 1:1 binding model using the FortéBio Data Analysis Software (version 11.1).

### ACE2 Competition by Biolayer Interferometry.

Antibody binding competition with recombinant human ACE2 receptor (Sino Biological, Cat #10108-H08H) was determined by BLI using a ForteBio Octet HTX (Sartorius). All binding steps were performed at 25° C. and at an orbital shaking speed of 1000 rpm. All reagents were formulated in PBSF (1×PBS with 0.1% w/v BSA). IgGs (100 nM) were captured onto anti-human IgG capture (AHC) biosensors (Molecular Devices) to a sensor response of 1.0 nm-1.4 nm, and then soaked (20 min) in an irrelevant IgG1 solution (0.5 mg/ml) to block remaining Fc binding sites. Next, sensors were equilibrated for 30 min in PBSF and then briefly exposed (90 s) to 300 nM of ACE2 to assess any potential cross interactions between sensor-loaded IgG and ACE2. Sensors were allowed to baseline (60 s) in PBSF before exposing (180 s) to 100 nM SARS-COV-2 RBD (Acro Biosystems, Cat #SPD-C52H3). Last, RBD-bound sensors were exposed (180 s) to 300 nM ACE2 to assess competition, where antibodies that resulted in increased sensor responses after ACE2 exposure represented non-ACE2-competitive binding profiles while those resulting in unchanged responses represented ACE2-competitive profiles.

### Deep Mutational Scanning Analysis of Antibody Binding Escape.

Yeast-display deep mutational scanning experiments identifying mutations that escape binding by each monoclonal antibody were conducted with duplicate site-saturation mutagenesis Omicron BA.1 RBD libraries (13). Yeast libraries were grown in SD-CAA media (6.7 g/L Yeast Nitrogen Base, 5.0 g/L Casamino acids, 2.13 g/L MES, and 2% w/v dextrose), and backdiluted to 0.67 OD600 in SG-CAA+0.1% D (SD-CAA with 2% galactose and 0.1% dextrose in place of the 2% dextrose) to induce RBD expression, which proceeded for 16-18 hours at room temperature with mild agitation. 5 OD of cells were washed in PBS-BSA (0.2 mg/L) and labeled for one hour at room temperature in 1 mL with a concentration of antibody determined as the EC90 from pilot isogenic binding assays. In parallel, 0.5 OD of yeast expressing the Omicron BA.1 wildtype RBD were incubated in 100 μL of antibody at the matched EC90 concentration or 0.1× the concentration for FACS gate-setting. Cells were washed, incubated with 1:100 FITC-conjugated chicken anti-Myc antibody (Immunology Consultants CMYC-45F) to label RBD expression and 1:200 PE-conjugated goat anti-human-IgG (Jackson ImmunoResearch 109-115-098) to label bound antibody. Labeled cells were washed and resuspended in PBS for FACS.

Antibody-escape cells in each library were selected via FACS on a BD FACSAria II. FACS selection gates were drawn to capture approximately 50% of yeast expressing the wildtype BA.1 RBD labeled at 10× reduced antibody labeling concentration (see gates in FIG. 27A). For each sample, ˜4 million RBD⁺ cells were processed on the sorter with collection of cells in the antibody-escape bin. Sorted cells were grown overnight in SD-CAA+pen-strep, plasmid purified (Zymo D2005), PCR amplified, and barcode sequenced on an Illumina NextSeq. In parallel, plasmid samples were purified from 30 OD of pre-sorted library cultures and sequenced to establish pre-selection barcode frequencies.

Demultiplexed Illumina barcode reads were matched to library barcodes in barcode-mutant lookup tables using dms_variants (version 0.8.9), yielding a table of counts of each barcode in each pre- and post-sort population. The escape fraction of each barcoded variant was computed from sequencing counts in the pre-sort and antibody-escape populations via the formula:

$${E}_{v} = F * ( \frac{{n}_{v}^{post}}{{N}^{post}} ) / ( \frac{{n}_{v}^{pre}}{{N}^{pre}} )$$

where F is the total fraction of the library that escapes antibody binding, nᵥ is the counts of variant v in the pre- or post-sort samples with a pseudocount addition of 0.5, and N is the total sequencing count across all variants pre- and post-sort. These escape fractions represent the estimated fraction of cells expressing a particular variant that fall in the escape bin.

Computational filters were applied to remove mutants with low sequencing counts or highly deleterious mutations that had ACE2 binding scores <−2 or expression scores of <−1, and mutations to the conserved RBD cysteine residues were removed. Per-mutant escape fractions were computed as the average across barcodes within replicates, with the correlations between replicate library selections shown in FIG. 27B.

### REFERENCES

1. WHO Coronavirus (COVID-19) Dashboard|WHO Coronavirus (COVID-19) Dashboard With Vaccination Data, (available at https://covid19.who.int/).
2. H. F. Tseng, B. K. Ackerson, Y. Luo, L. S. Sy, C. A. Talarico, Y. Tian, K. J. Bruxvoort, J. E. Tubert, A. Florea, J. H. Ku, G. S. Lee, S. K. Choi, H. S. Takhar, M. Aragones, L. Qian, Effectiveness of mRNA-1273 against SARS-COV-2 Omicron and Delta variants. Nat. Med. 28, 1063-1071 (2022).
3. S. Y. Tartof, J. M. Slezak, L. Puzniak, V. Hong, F. Xie, B. K. Ackerson, S. R. Valluri, L. Jodar, J. M. McLaughlin, Durability of BNT162b2 vaccine against hospital and emergency department admissions due to the omicron and delta variants in a large health system in the USA: a test-negative case-control study. Lancet Respir. Med. 10, 689-699 (2022).
4. Q. Wang, Y. Guo, S. Iketani, M. S. Nair, Z. Li, H. Mohri, M. Wang, J. Yu, A. D. Bowen, J. Y. Chang, J. G. Shah, N. Nguyen, Z. Chen, K. Meyers, M. T. Yin, M. E. Sobieszczyk, Z. Sheng, Y. Huang, L. Liu, D. D. Ho, Antibody evasion by SARS-COV-2 Omicron subvariants BA.2.12.1, BA.4 and BA.5. Nature. 608, 603-608 (2022).
5. A. Tuekprakhon, R. Nutalai, A. Dijokaite-Guraliuc, D. Zhou, H. M. Ginn, M. Selvaraj, C. Liu, A. J. Mentzer, P. Supasa, H. M. E. Duyvesteyn, R. Das, D. Skelly, T. G. Ritter, A. Amini, S. Bibi, S. Adele, S. A. Johnson, B. Constantinides, H. Webster, N. Temperton, G. R. Screaton, Antibody escape of SARS-COV-2 Omicron BA.4 and BA.5 from vaccine and BA.1 serum. Cell. 185, 2422-2433.e13 (2022).
6. Y. Cao, F. Jian, J. Wang, Y. Yu, W. Song, A. Yisimayi, J. Wang, R. An, N. Zhang, Y. Wang, P. Wang, L. Zhao, H. Sun, L. Yu, S. Yang, X. Niu, T. Xiao, Q. Gu, F. Shao, X. Hao, X. S. Xie, Imprinted SARS-COV-2 humoral immunity induces converging Omicron RBD evolution. BioRxiv (2022), doi: 10.1101/2022.09.15.507787.
7. D. J. Sheward, C. Kim, J. Fischbach, S. Muschiol, R. A. Ehling, N. K. Björkström, G. B. Karlsson Hedestam, S. T. Reddy, J. Albert, T. P. Peacock, B. Murrell, Omicron sublineage BA.2.75.2 exhibits extensive escape from neutralising antibodies. BioRxiv (2022), doi: 10.1101/2022.09.16.508299.
8. Coronavirus (COVID-19) Update: FDA Authorizes Moderna, Pfizer-BioNTech Bivalent COVID-19 Vaccines for Use as a Booster Dose|FDA, (available at https://www.fda.gov/news-events/press-announcements/coronavirus-covid-19-update-fda-authorizes-moderna-pfizer-biontech-bivalent-covid-19-vaccines-use).
9. C. I. Kaku, A. J. Bergeron, C. Ahlm, J. Normark, M. Sakharkar, M. N. E. Forsell, L. M. Walker, Recall of preexisting cross-reactive B cell memory after Omicron BA.1 breakthrough infection. Sci. Immunol. 7, eabq3511 (2022).
10. J. Quandt, A. Muik, N. Salisch, B. G. Lui, S. Lutz, K. Krüger, A.-K. Wallisch, P. Adams-Quack, M. Bacher, A. Finlayson, O. Ozhelvaci, I. Vogler, K. Grikscheit, S. Hoehl, U.
Goetsch, S. Ciesek, Ö. Türeci, U. Sahin, Omicron BA.1 breakthrough infection drives cross-variant neutralization and memory B cell formation against conserved epitopes. Sci. Immunol. 7, eabq2427 (2022).
11. Z. Wang, P. Zhou, F. Muecksch, A. Cho, T. B. Tanfous, M. Canis, L. Witte, B. Johnson, R. Raspe, F. Schmidt, E. Bednarski, J. D. Silva, V. Ramos, S. Zong, M. Turroja, K. G. Millard, K.-H. Yao, I. Shimeliovich, J. Dizon, A. Kaczynska, M. C. Nussenzweig, Memory B cell responses to Omicron subvariants after SARS-COV-2 mRNA breakthrough infection. BioRxiv (2022), doi: 10.1101/2022.08.11.503601.
12. S. Chalkias, C. Harper, K. Vrbicky, S. R. Walsh, B. Essink, A. Brosz, N. McGhee, J. E. Tomassini, X. Chen, Y. Chang, A. Sutherland, D. C. Montefiori, B. Girard, D. K. Edwards, J. Feng, H. Zhou, L. R. Baden, J. M. Miller, R. Das, A Bivalent Omicron-Containing Booster Vaccine against Covid-19. N. Engl. J. Med. (2022), doi: 10.1056/NEJMoa2208343.
13. A. Schiepers, M. F. L. van't Wout, A. J. Greaney, T. Zang, H. Muramatsu, P. J. C. Lin, Y. K. Tam, L. Mesin, T. N. Starr, P. D. Bieniasz, N. Pardi, J. D. Bloom, G. D. Victora, Molecular fate-mapping of serum antibodies reveals the effects of antigenic imprinting on repeated immunization. BioRxiv (2022), doi: 10.1101/2022.08.29.505743.
14. H. Chemaitelly, H. H. Ayoub, P. Coyle, P. Tang, H. M. Yassine, H. A. Al-Khatib, M. K. Smatti, M. R. Hasan, Z. Al-Kanaani, E. Al-Kuwari, A. Jeremijenko, A. H. Kaleeckal, A. N. Latif, R. M. Shaik, H. F. Abdul-Rahim, G. K. Nasrallah, M. G. Al-Kuwari, A. A. Butt, H. E. Al-Romaihi, M. H. Al-Thani, L. J. Abu-Raddad, Protection of Omicron sub-lineage infection against reinfection with another Omicron sub-lineage. medRxiv (2022), doi: 10.1101/2022.02.24.22271440.
15. J. Malato, R. M. Ribeiro, P. P. Leite, P. Casaca, E. Fernandes, C. Antunes, V. R. Fonseca, M. C. Gomes, L. Graca, Risk of BA.5 Infection among Persons Exposed to Previous SARS-COV-2 Variants. N. Engl. J. Med. 387, 953-954 (2022).
16. D.-Y. Lin, Y. Gu, Y. Xu, D. Zeng, B. Wheeler, H. Young, S. K. Sunny, Z. Moore, Effects of vaccination and previous infection on omicron infections in children. N. Engl. J. Med. (2022), doi: 10.1056/NEJMc2209371.
17. F. Muecksch, Z. Wang, A. Cho, C. Gaebler, T. Ben Tanfous, J. DaSilva, E. Bednarski, V. Ramos, S. Zong, B. Johnson, R. Raspe, D. Schaefer-Babajew, I. Shimeliovich, M. Daga, K.-H. Yao, F. Schmidt, K. G. Millard, M. Turroja, M. Jankovic, T. Y. Oliveira, M. C. Nussenzweig, Increased memory B cell potency and breadth after a SARS-COV-2 mRNA boost. Nature. 607, 128-134 (2022).
18. W. F. Garcia-Beltran, K. J. St Denis, A. Hoelzemer, E. C. Lam, A. D. Nitido, M. L. Sheehan, C. Berrios, O. Ofoman, C. C. Chang, B. M. Hauser, J. Feldman, A. L. Roederer, D. J. Gregory, M. C. Poznansky, A. G. Schmidt, A. J. Iafrate, V. Naranbhai, A. B. Balazs, mRNA-based COVID-19 vaccine boosters induce neutralizing immunity against SARS-COV-2 Omicron variant. Cell. 185, 457-466.e4 (2022).
19. D. F. Robbiani, C. Gaebler, F. Muecksch, J. C. C. Lorenzi, Z. Wang, A. Cho, M. Agudelo, C. O. Barnes, A. Gazumyan, S. Finkin, T. Hägglöf, T. Y. Oliveira, C. Viant, A. Hurley, H.-H. Hoffmann, K. G. Millard, R. G. Kost, M. Cipolla, K. Gordon, F. Bianchini, M. C. Nussenzweig, Convergent antibody responses to SARS-COV-2 in convalescent individuals. Nature. 584, 437-442 (2020).
20. C. O. Barnes, C. A. Jette, M. E. Abernathy, K.-M. A. Dam, S. R. Esswein, H. B. Gristick, A. G. Malyutin, N. G. Sharaf, K. E. Huey-Tubman, Y. E. Lee, D. F. Robbiani, M. C. Nussenzweig, A. P. West, P. J. Bjorkman, SARS-COV-2 neutralizing antibody structures inform therapeutic strategies. Nature. 588, 682-687 (2020).
21. Z. Wang, F. Schmidt, Y. Weisblum, F. Muecksch, C. O. Barnes, S. Finkin, D. Schaefer-Babajew, M. Cipolla, C. Gaebler, J. A. Lieberman, T. Y. Oliveira, Z. Yang, M. E. Abernathy, K. E. Huey-Tubman, A. Hurley, M. Turroja, K. A. West, K. Gordon, K. G. Millard, V. Ramos, M. C. Nussenzweig, mRNA vaccine-elicited antibodies to SARS-COV-2 and circulating variants. Nature. 592, 616-622 (2021).
22. A. Lanzavecchia, A. Frühwirth, L. Perez, D. Corti, Antibody-guided vaccine design: identification of protective epitopes. Curr. Opin. Immunol. 41, 62-67 (2016).
23. A. B. Ward, I. A. Wilson, Innovations in structure-based antigen design and immune monitoring for next generation vaccines. Curr. Opin. Immunol. 65, 50-56 (2020).
24. P. D. Kwong, J. R. Mascola, HIV-1 Vaccines Based on Antibody Identification, B Cell Ontogeny, and Epitope Structure. Immunity. 48, 855-871 (2018).
25. B. Briney, A. Inderbitzin, C. Joyce, D. R. Burton, Commonality despite exceptional diversity in the baseline human antibody repertoire. Nature. 566, 393-397 (2019).
26. T. F. Rogers, F. Zhao, D. Huang, N. Beutler, A. Burns, W.-T. He, O. Limbo, C. Smith, G. Song, J. Woehl, L. Yang, R. K. Abbott, S. Callaghan, E. Garcia, J. Hurtado, M. Parren, L. Peng, S. Ramirez, J. Ricketts, M. J. Ricciardi, D. R. Burton, Isolation of potent SARS-COV-2 neutralizing antibodies and protection from disease in a small animal model. Science. 369, 956-963 (2020).
27. A. Z. Wec, D. Haslwanter, Y. N. Abdiche, L. Shehata, N. Pedreño-Lopez, C. L. Moyer, Z. A. Bornholdt, A. Lilov, J. H. Nett, R. K. Jangra, M. Brown, D. I. Watkins, C. Ahlm, M. N. Forsell, F. A. Rey, G. Barba-Spaeth, K. Chandran, L. M. Walker, Longitudinal dynamics of the human B cell response to the yellow fever 17D vaccine. Proc Natl Acad Sci USA. 117, 6675-6685 (2020).
28. R. D. Gietz, R. H. Schiestl, High-efficiency yeast transformation using the LiAc/SS carrier DNA/PEG method. Nat. Protoc. 2, 31-34 (2007).
29. M. I. J. Raybould, A. Kovaltsuk, C. Marks, C. M. Deane, CoV-AbDab: the coronavirus antibody database. Bioinformatics. 37, 734-735 (2021).
30. M. Sakharkar, C. G. Rappazzo, W. F. Wieland-Alter, C.-L. Hsieh, D. Wrapp, E. S. Esterman, C. I. Kaku, A. Z. Wec, J. C. Geoghegan, J. S. Mclellan, R. I. Connor, P. F. Wright, L. M. Walker, Prolonged evolution of the human B cell response to SARS-COV-2 infection. Sci. Immunol. 6 (2021), doi: 10.1126/sciimmunol.abg6916.

### Example 3. Re-Engineering of Antibody VYD223

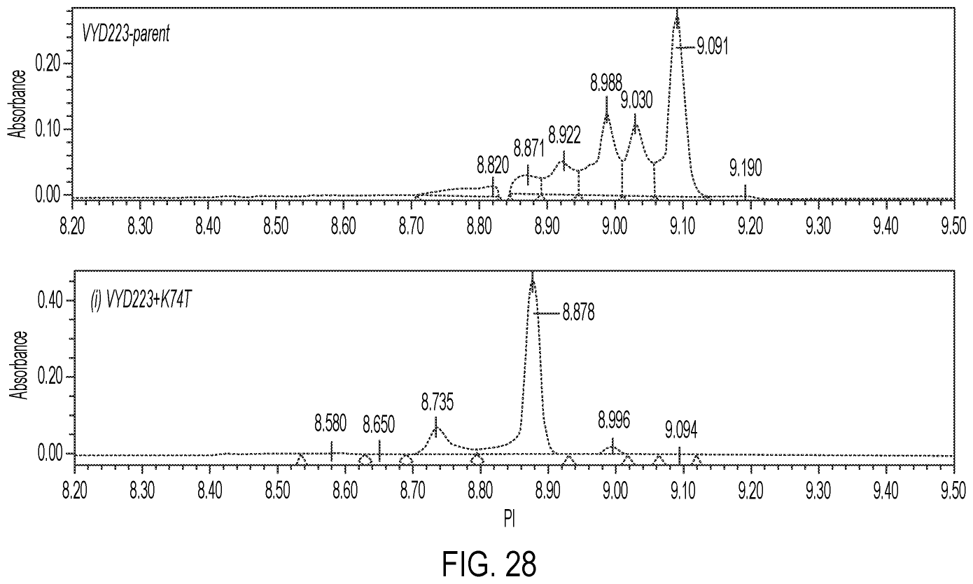

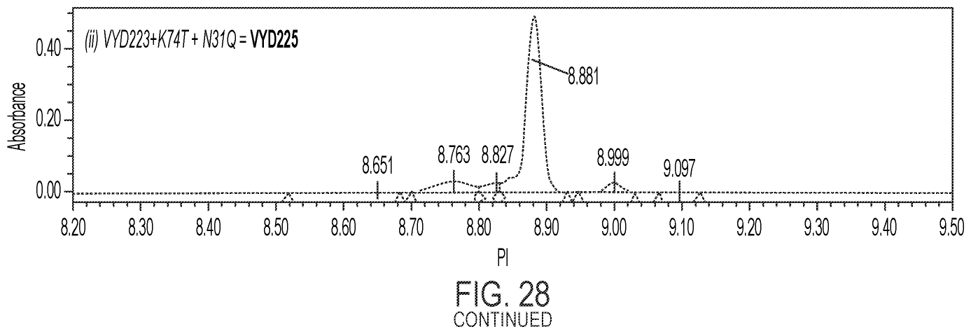

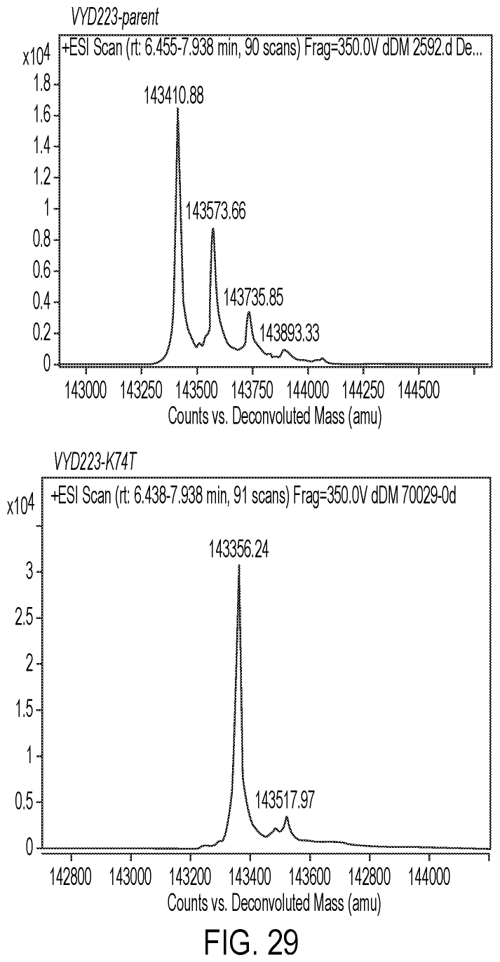

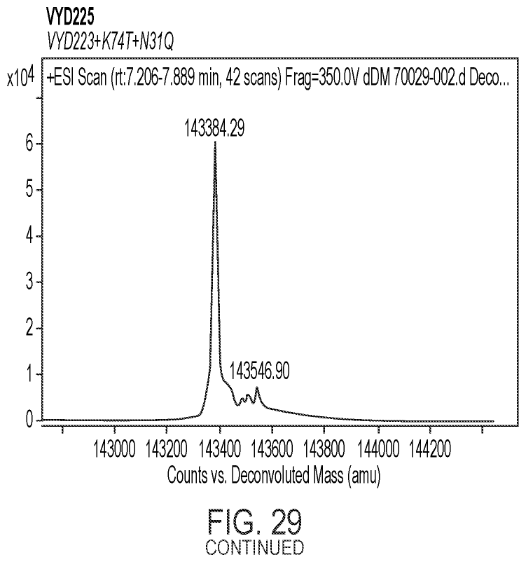

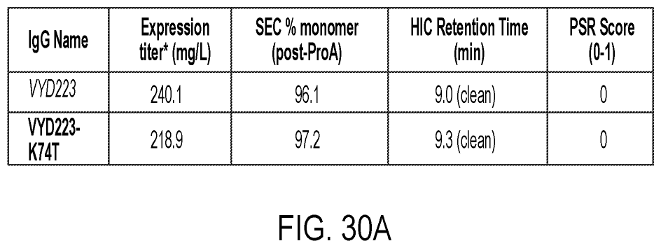

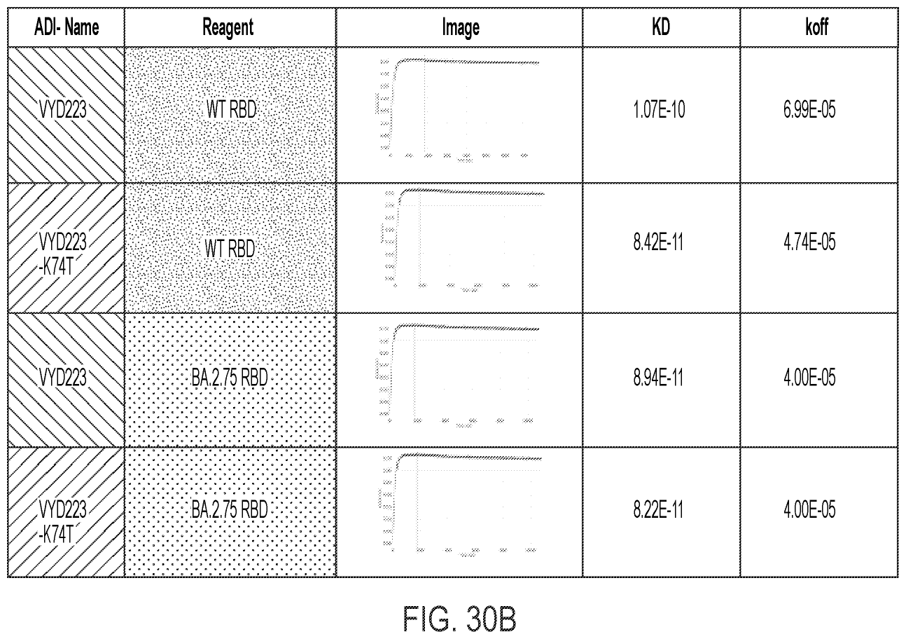

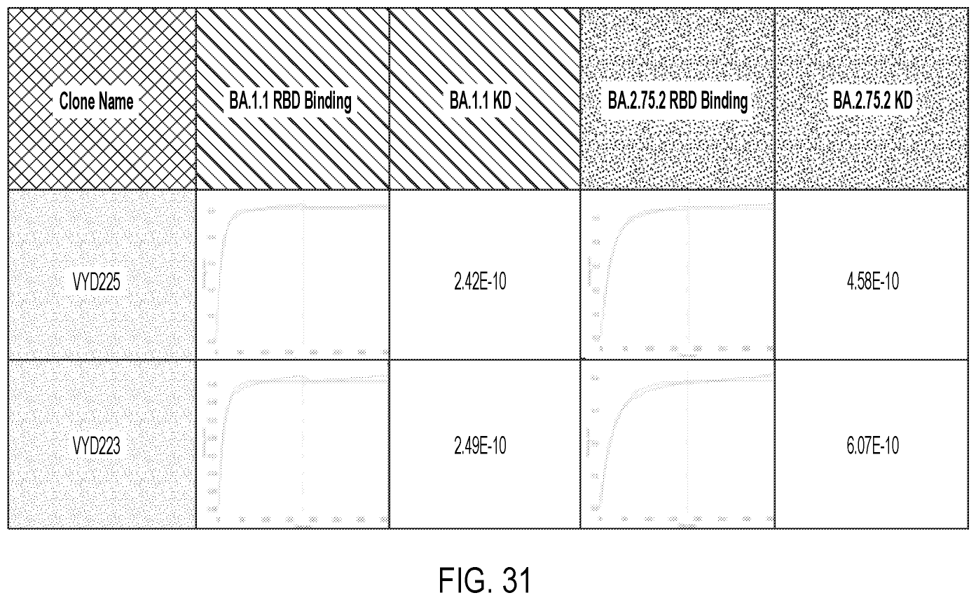

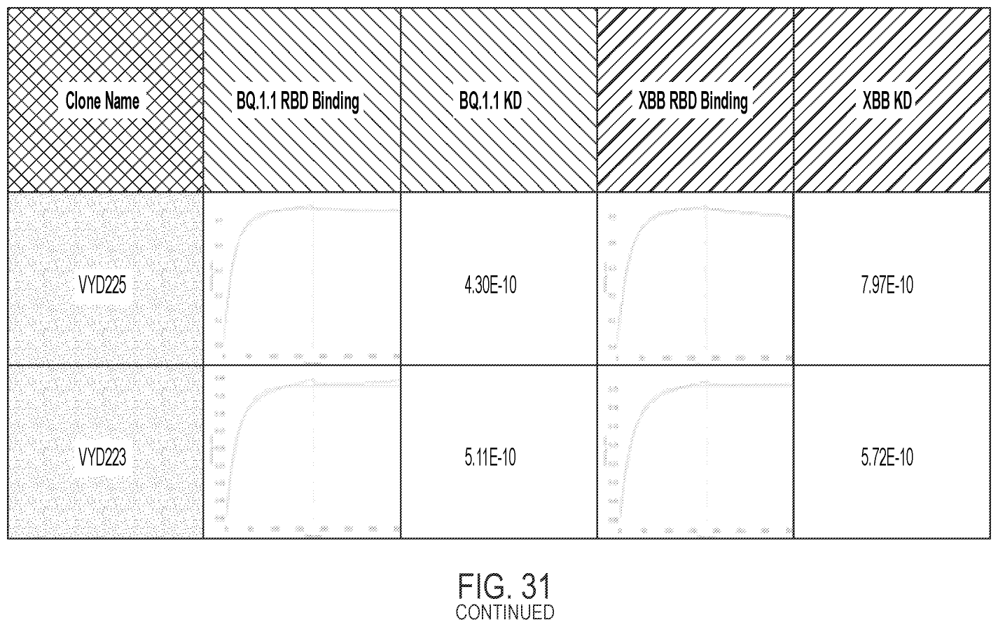

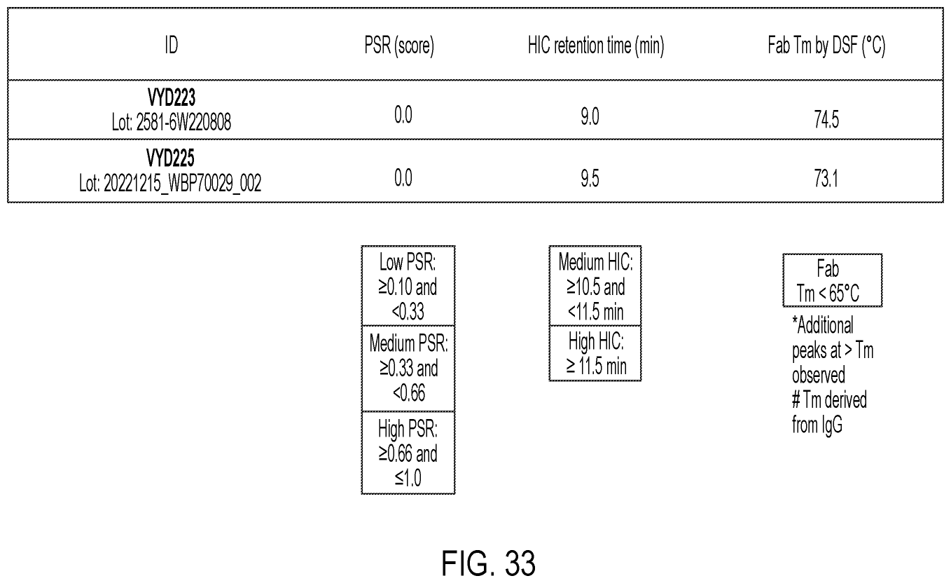

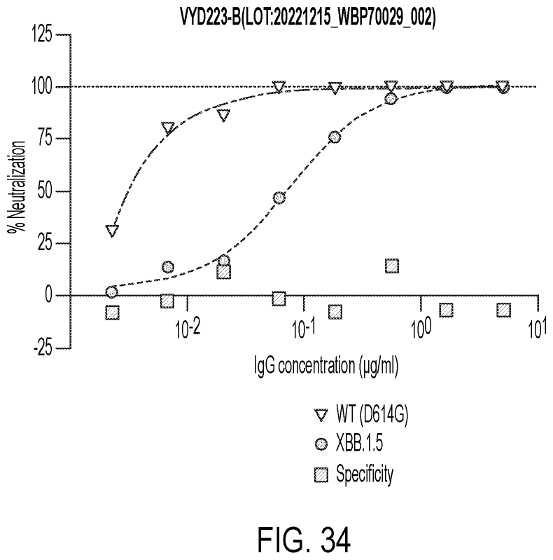

Antibody VYD223 (also known as ADI-75865) was re-engineered by modifying the glycation and deamination sites in the heavy chain. Specifically, K74T and N31Q substitutions were introduced to the heavy chain of VYD223. The resulting antibody was named as VYD225. VYD225 shows a highly similar neutralization potency to the parental mAb when produced in an identical process, yet with improved developability characteristics. Specifically, VYD225 had a significant reduction in acidic species (FIG. 28) and glycated species (FIG. 29) when compared to VYD223. In addition, removal of glycation site (K74T) showed no negative impact on biophysical properties (FIG. 30A) and binding activity (FIG. 30B). The biophysical properties of VYD225 and VYD223 were also shown in FIG. 33. FIG. 34 depicts the neutralization activity of VYD223 against WT SARS-COV-2 and the XBB.1.5 variant.

To evaluate the binding properties of VYD225 and the parental mAb, VYD223, their monovalent binding affinities for the RBD of the wild type SARS-COV-2 strain, the BA.1.1 variant, the BQ.1.1 variant, the BA.2.75 variant, the XBB.1 variant were measured.

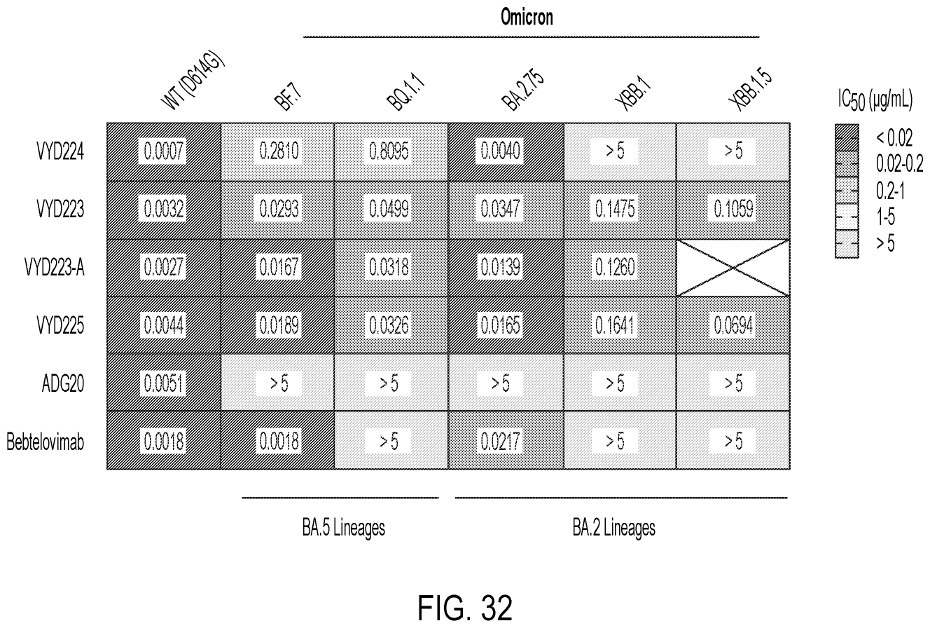

In addition, the neutralizing activity of VYD225 against the wild type SARS-COV-2 strain, the BF.7 variant, the BQ.1.1 variant, the BA.2.75 variant, the XBB.1 variant, and the XBB.1.5 variant was assessed using an pseudovirus assay, and compared with other mAbs, e.g., VYD223, VYD224, and adintrevimab (also known as ADI-58125). As shown in FIG. 32, VYD225 demonstrates potent and broad pseudovirus neutralization potency against all variants tested including XBB.1 and XBB1.5.

These data suggest that VYD225 displays broad activity against the SARS-COV-2 VOCs, including the Omicron variants. This antibody represents a promising candidate for therapeutic development and provides a framework for the development of vaccines that induce broadly neutralizing antibody responses.

### Example 4. In Vitro Pseudovirus Neutralization Assay Using SARS-COV-2 Spike Glycoprotein Variants

SARS-COV-2 pseudovirus neutralization assays were performed using the PhenoSense SARS-CoV-2 Neutralizing Antibody Assay (Monogram Biosciences), as described in Huang et al., 2021, Sci Rep. 11 (1): 23921. Briefly, pseudoviruses bearing SARS-COV-2 D614G or variant spike proteins were produced by co-transfecting HEK293 cells with a codon-optimized spike sequence expression vector and an HIV genomic vector with a firefly luciferase reporter gene replacing the HIV envelope gene. Culture supernatants were harvested 48 hours post transfection, filtered, and frozen at <−70° C. Pseudovirus titers were determined by inoculating HEK293T cells that were transiently transfected with hACE2 and TMPRSS2 expression vectors and measuring luciferase activity (in RLU) following incubation at 37° C. for 3 days. Virus inoculum for the assay was standardized for all variants based on the screening RLUs.

To test antibody neutralization, a predetermined amount of pseudovirus was incubated with titrating amounts of test mAb for 1 hour at 37° C. before adding to HEK293 cells expressing hACE2 and TMPRSS2. For each variant tested, the WT (D614G) reference was tested in parallel. Pseudovirus infection was allowed to occur for 3 days before cells were assessed for luciferase activity. Luciferase activity was determined by adding Steady Glo (Promega) and measuring luciferase signal (RLU) using a luminometer.

The neutralization potency of VYD224 (also known as ADI-80707) was assessed against SARS-COV-2 variant pseudoviruses in the PhenoSense SARS-COV-2 neutralizing assay. As shown in Table 8, VYD224 demonstrated potency against all pseudovirus variants except for XBB, XBB.1 and XBB.1.5, against which it lacked activity.

TABLE 8
Neutralization Activity of VYD224 Against SARS-CoV-2 Pseudovirus

| Pango | Variant | RBD Substitutions Present | IC₅₀ (μg/mL) |
|---|---|---|---|
| Lineage | Description | in Monogram Pseudovirus | VYD224 |
| B.1 | WT (D614G) | | 0.0005-0.0011 |
| B.1.617.2 | Delta | L452R, T478K | 0.0010 |
| BA.1 | Omicron | G339D, S371L, S373P, S375F, K417N, | 0.0013 |
| | | N440K, G446S, S477N, T478K, E484A, | |
| | | Q493K, G496S, Q498R, N501Y, Y505H | |
| BA.4/5 | Omicron | G339D, S371F, S373P, S375F, T376A, | 0.4002 |
| | | D405N, R408S, K417N, N440K, L452R, | |
| | | S477N, T478K, E484A, F486V, Q498R, | |
| | | N501Y, Y505H | |
| BA.4.6 | Omicron | G339D, R346T, S371F, S373P, S375F, | 0.2667 |
| | | T376A, D405N, R408S, K417N, N440K, | |
| | | L452R, S477N, T478K, E484A, F486V, | |
| | | Q498R, N501Y, Y505H | |
| BF.7 | Omicron | G339D, R346T, S371F, S373P, S375F, | 0.2810 |
| | | T376A, D405N, R408S, K417N, N440K, | |
| | | L452R, S477N, T478K, E484A, F486V, | |
| | | Q498R, N501Y, Y505H | |
| BQ.1 | Omicron | G339D, S371F, S373P, S375F, T376A, | 0.4402 |
| | | D405N, R408S, K417N, N440K, K444T, | |
| | | L452R, N460K, S477N, T478K, E484A, | |
| | | F486V, Q498R, N501Y, Y505H | |
| BQ.1.1 | Omicron | G339D, R346T, S371F, S373P, S375F, | 0.8095 |
| | | T376A, D405N, R408S, K417N, N440K, | |
| | | K444T, L452R, N460K, S477N, T478K, | |
| | | E484A, F486V, Q498R, N501Y, Y505H | |
| BA.2.75 | Omicron | G339H, S371F, S373P, S375F, T376A, | 0.0040 |
| | | D405N, R408S, K417N, N440K, G446S, | |
| | | N460K, S477N, T478K, E484A, Q498R, | |
| | | N501Y, Y505H | |
| BN.1 | Omicron | G339H, R346T, K356T, S371F, S373P, | 0.0015 |
| | | S375F, T376A, D405N, R408S, K417N, | |
| | | N440K, G446S, N460K, S477N, T478K, | |
| | | E484A, F490S, Q498R, N501Y, Y505H | |
| XBB | Omicron | G339H, R346T, L368I, S371F, S373P, | >5 |
| | | S375F, T376A, D405N, R408S, K417N, | |
| | | N440K, V445P, G446S, N460K, S477N, | |
| | | T478K, E484A, F486S, F490S, Q493R, | |
| | | Q498R, N501Y, Y505H | |
| XBB.1 | Omicron | G339H, R346T, L368I, S371F, S373P, | >5 |
| | | S375F, T376A, D405N, R408S, K417N, | |
| | | N440K, V445P, G446S, N460K, S477N, | |
| | | T478K, E484A, F486S, F490S, Q493R, | |
| | | Q498R, N501Y, Y505H | |
| XBB.1.5 | Omicron | G339H, R346T, L368I, S371F, S373P, | >5 |
| | | S375F, T376A, D405N, R408S, K417N, | |
| | | N440K, V445P, G446S, N460K, S477N, | |
| | | T478K, E484A, F486P, F490S, Q493R, | |
| | | Q498R, N501Y, Y505H | |

IC50 = half-maximal inhibitory concentration; SARS-CoV-2 = severe acute respiratory syndrome coronavirus 2; WT = wild-type. All data were generated using the PhenoSense SARS-CoV-2 Neutralizing Antibody Assay (Monogram Biosciences, Inc). IC50 values are reported as a range when data were obtained from independent experiments. 5 μg/mL was the upper concentration tested.

Having fully described and enabled the invention, the invention is further described by the claims that follow.

TABLE 3

| | VH | | SEQ | | SEQ | | SEQ | | SEQ | | SEQ | | SEQ | | SEQ | | SEQ | | SEQ | Reverted | SEQ |
|---|---|---|---|---|---|---|---|---|---|---|---|---|---|---|---|---|---|---|---|---|---|
| Clone | Germ- | | ID | VH | ID | VH | ID | VH | ID | VH | ID | VH | ID | VH | ID | | ID | | ID | VH | ID |
| ID | line | VH FR1 | NO: | CDR1 | NO: | FR2 | NO: | CDR2 | NO: | FR3 | NO: | CDR3 | NO: | FR4 | NO: | VH DNA | NO: | VH Protein | NO: | Protein | NO: |
| ADI- | IGHV3- | QVQLVQSG | 9 | FTFRSYG | 198 | WVRQA | 431 | DILYDG | 519 | RFTIS | 777 | AKQSGP | 1021 | WGQGAL | 1285 | CAGGTCCAGCTGGTGCAGTCTGGGGGA | 1306 | QVQLVQSGGA | 1589 | | |
| 75846 | 30 | GAVVQPGR | | MH | | PGKGL | | SNKYYA | | RDNSK | | YCSGRS | | VTVSS | | GCCGTGGTCCAGCCTGGGAGGTCCCTG | | VVQPGRSLRL | | | |
| | | SLRLSCAA | | | | EWVA | | DSVKG | | NTLYL | | CYSGLF | | | | AGACTCTCCTGTGCAGCCTCTGGATTC | | SCAASGFTFR | | | |
| | | SG | | | | | | | | QMNSL | | DY | | | | ACCTTCCGAAGTTATGGCATGCACTGG | | SYGMHWVRQA | | | |
| | | | | | | | | | | RAEDT | | | | | | GTCCGCCAGGCTCCAGGCAAGGGGCTG | | PGKGLEWVAD | | | |
| | | | | | | | | | | AVYYC | | | | | | GAGTGGGTGGCAGATATATTATATGAT | | ILYDGSNKYY | | | |
| | | | | | | | | | | | | | | | | GGAAGTAATAAATACTATGCAGACTCC | | ADSVKGRFTI | | | |
| | | | | | | | | | | | | | | | | GTGAAGGGCCGATTCACCATCTCCAGA | | SRDNSKNTLY | | | |
| | | | | | | | | | | | | | | | | GACAATTCCAAGAACACGCTGTATCTG | | LQMNSLRAED | | | |
| | | | | | | | | | | | | | | | | CAAATGAACAGCCTGAGAGCTGAGGAC | | TAVYYCAKQS | | | |
| | | | | | | | | | | | | | | | | ACGGCTGTATATTACTGTGCGAAACAG | | GPYCSGRSCY | | | |
| | | | | | | | | | | | | | | | | TCCGGCCCTTATTGTAGTGGTCGTAGC | | SGLFDYWGQG | | | |
| | | | | | | | | | | | | | | | | TGCTACTCGGGTCTCTTTGACTACTGG | | ALVTVSS | | | |
| | | | | | | | | | | | | | | | | GGCCAGGGAGCCCTGGTCACCGTCTCC | | | | | |
| | | | | | | | | | | | | | | | | TCA | | | | | |
| ADI- | IGHV3- | QVQLVQSG | 10 | FTFSSYA | 199 | WVRQA | 431 | VISDDG | 520 | RFTIS | 778 | ARDLNG | 1022 | WGQGTL | 1286 | CAGGTCCAGCTGGTGCAGTCTGGGGGA | 1307 | QVQLVQSGGG | 1590 | | |
| 75829 | 30 | GGVVQPGR | | MH | | PGKGL | | NNKEYP | | RDNSK | | DFFDFA | | VTVSS | | GGCGTGGTCCAGCCTGGGAGGTCCCTG | | VVQPGRSLRL | | | |
| | | SLRLSCAA | | | | EWVA | | DSVKG | | NTLYL | | IFDY | | | | AGACTCTCCTGTGCAGCCTCTGGATTC | | SCAASGFTFS | | | |
| | | SG | | | | | | | | QVNSL | | | | | | ACCTTCAGCAGTTATGCTATGCACTGG | | SYAMHWVRQA | | | |
| | | | | | | | | | | TAEDT | | | | | | GTCCGCCAGGCCCCAGGCAAGGGGCTG | | PGKGLEWVAV | | | |
| | | | | | | | | | | AVYYC | | | | | | GAGTGGGTGGCAGTTATATCAGATGAT | | ISDDGNNKEY | | | |
| | | | | | | | | | | | | | | | | GGAAACAATAAAGAGTACCCAGACTCC | | PDSVKGRFTI | | | |
| | | | | | | | | | | | | | | | | GTGAAGGGCCGCTTCACCATCTCCAGA | | SRDNSKNTLY | | | |
| | | | | | | | | | | | | | | | | GACAATTCCAAGAACACACTATATCTG | | LQVNSLTAED | | | |
| | | | | | | | | | | | | | | | | CAAGTGAACAGCCTGACAGCTGAGGAC | | TAVYYCARDL | | | |
| | | | | | | | | | | | | | | | | ACGGCTGTTTATTACTGTGCGAGAGAT | | NGDFFDFAIF | | | |
| | | | | | | | | | | | | | | | | CTTAACGGTGACTTCTTTGACTTCGCG | | DYWGQGTLVT | | | |
| | | | | | | | | | | | | | | | | ATTTTTGACTACTGGGGCCAGGGAACC | | VSS | | | |
| | | | | | | | | | | | | | | | | CTGGTCACCGTCTCCTCA | | | | | |
| ADI- | IGHV3- | QVQLVESG | 11 | FTFTSYG | 200 | WVRQA | 431 | VISNDG | 521 | RFTIS | 779 | ARDYGD | 1023 | WGQGTL | 1286 | CAGGTCCAGCTGGTGGAGTCTGGGGGA | 1308 | QVQLVESGGG | 1591 | | |
| 75811 | 30 | GGVVQPGR | | FH | | PGKGL | | SYKDYV | | RDNSK | | FVTWFD | | VTVSS | | GGCGTGGTCCAGCCAGGGAGGTCCCTG | | VVQPGRSLRL | | | |
| | | SLRLSCAA | | | | EWVA | | DSVKG | | NTVYL | | P | | | | AGACTCTCCTGTGCAGCCTCTGGATTC | | SCAASGFTFT | | | |
| | | SG | | | | | | | | QMNSL | | | | | | ACCTTCACTAGTTATGGTTTCCACTGG | | SYGFHWVRQA | | | |
| | | | | | | | | | | RAEDT | | | | | | GTCCGCCAGGCTCCAGGCAAGGGTCTA | | PGKGLEWVAV | | | |
| | | | | | | | | | | AVYYC | | | | | | GAGTGGGTGGCAGTTATATCAAATGAT | | ISNDGSYKDY | | | |
| | | | | | | | | | | | | | | | | GGAAGTTACAAAGACTACGTAGACTCC | | VDSVKGRFTI | | | |
| | | | | | | | | | | | | | | | | GTGAAGGGCCGATTCACCATCTCCAGA | | SRDNSKNTVY | | | |
| | | | | | | | | | | | | | | | | GACAATTCCAAGAACACGGTGTATCTC | | LQMNSLRAED | | | |
| | | | | | | | | | | | | | | | | CAAATGAACAGCCTGAGAGCTGAGGAC | | TAVYYCARDY | | | |
| | | | | | | | | | | | | | | | | ACGGCTGTGTATTACTGTGCGAGAGAC | | GDFVTWFDPW | | | |
| | | | | | | | | | | | | | | | | TACGGTGACTTCGTCACCTGGTTCGAC | | GQGTLVTVSS | | | |
| | | | | | | | | | | | | | | | | CCCTGGGGCCAGGGAACCCTGGTCACC | | | | | |
| | | | | | | | | | | | | | | | | GTCTCCTCA | | | | | |
| ADI- | IGHV3- | QVQLVQSG | 12 | FTFTTYW | 201 | WVRQA | 431 | SIKQDG | 522 | RFTIS | 780 | AREMGL | 1024 | WGQGTL | 1286 | CAGGTGCAGCTGGTGCAGTCTGGGGGA | 1309 | QVQLVQSGGG | 1592 | | |
| 75585 | 7 | GGLVQPGG | | MS | | PGKGL | | SEAYYV | | RDNAR | | FWFDP | | VTVSS | | GGCTTGGTCCAGCCTGGGGGGTCCCTG | | LVQPGGSLRL | | | |
| | | SLRLSCAA | | | | EWVA | | DSVRG | | NSLHL | | | | | | AGACTCTCCTGTGCAGCCTCTGGATTC | | SCAASGFTFT | | | |
| | | SG | | | | | | | | QMISL | | | | | | ACCTTTACTACCTATTGGATGAGCTGG | | TYWMSWVRQA | | | |
| | | | | | | | | | | RAEDT | | | | | | GTCCGCCAGGCTCCAGGGAAGGGGCTG | | PGKGLEWVAS | | | |
| | | | | | | | | | | AVYYC | | | | | | GAGTGGGTGGCCAGTATAAAGCAAGAT | | IKQDGSEAYY | | | |
| | | | | | | | | | | | | | | | | GGAAGTGAGGCATATTATGTGGACTCT | | VDSVRGRFTI | | | |
| | | | | | | | | | | | | | | | | GTGAGGGGCCGATTCACCATCTCCAGA | | SRDNARNSLH | | | |
| | | | | | | | | | | | | | | | | GACAACGCCAGGAACTCACTGCATCTG | | LQMISLRAED | | | |
| | | | | | | | | | | | | | | | | CAAATGATCAGCCTGAGAGCCGAGGAC | | TAVYYCAREM | | | |
| | | | | | | | | | | | | | | | | ACGGCTGTGTATTATTGTGCGAGAGAA | | GLFWFDPWGQ | | | |
| | | | | | | | | | | | | | | | | ATGGGTTTATTCTGGTTCGACCCCTGG | | GTLVTVSS | | | |
| | | | | | | | | | | | | | | | | GGCCAGGGAACCCTGGTCACCGTCTCC | | | | | |
| | | | | | | | | | | | | | | | | TCA | | | | | |
| ADI- | IGHV3- | QVQLVQSG | 13 | FSFSDAW | 202 | WVRQA | 432 | RISSET | 523 | RFTIS | 781 | STDDVL | 1025 | WGQGTT | 1287 | CAGGTGCAGCTGGTGCAGTCTGGGGGA | 1310 | QVQLVQSGGG | 1593 | | |
| 75869 | 15 | GGLVKPGG | | MS | | PGKGL | | DGGTTE | | RDNSK | | TGYDYA | | VTVSS | | GGCTTGGTAAAGCCTGGGGGGTCCCTT | | LVKPGGSLRL | | | |
| | | SLRLSCAA | | | | EWVG | | YAAPVK | | GTLYL | | MDV | | | | AGACTCTCCTGTGCAGCCTCTGGATTC | | SCAASGFSFS | | | |
| | | SG | | | | | | G | | QMNSL | | | | | | AGTTTCAGTGACGCCTGGATGAGCTGG | | DAWMSWVRQA | | | |
| | | | | | | | | | | KTEDT | | | | | | GTCCGCCAGGCTCCAGGGAAGGGGCTG | | PGKGLEWVGR | | | |
| | | | | | | | | | | AVYYC | | | | | | GAGTGGGTTGGCCGTATCAGCAGCGAA | | ISSETDGGTT | | | |
| | | | | | | | | | | | | | | | | ACTGATGGTGGGACAACAGAGTACGCT | | EYAAPVKGRF | | | |
| | | | | | | | | | | | | | | | | GCACCCGTGAAAGGCAGATTCACCATC | | TISRDNSKGT | | | |
| | | | | | | | | | | | | | | | | TCAAGAGATAATTCAAAAGGCACGCTG | | LYLQMNSLKT | | | |
| | | | | | | | | | | | | | | | | TATCTGCAAATGAACAGCCTGAAAACC | | EDTAVYYCST | | | |
| | | | | | | | | | | | | | | | | GAGGACACAGCCGTGTATTACTGTTCC | | DDVLTGYDYA | | | |
| | | | | | | | | | | | | | | | | ACTGACGATGTTTTGACTGGCTATGAC | | MDVWGQGTTV | | | |
| | | | | | | | | | | | | | | | | TACGCTATGGACGTCTGGGGCCAAGGG | | TVSS | | | |
| | | | | | | | | | | | | | | | | ACCACGGTCACCGTCTCCTCA | | | | | |
| ADI- | IGHV1- | QVQLQQSG | 14 | YIFINYY | 203 | WVRQA | 433 | IISPRS | 524 | RVTMS | 782 | ARDRRS | 1026 | WGQGTL | 1286 | CAGGTGCAGCTGCAGCAGTCTGGGACT | 1311 | QVQLQQSGTE | 1594 | | |
| 75817 | 46 | TEVKKPGA | | IS | | PGQGL | | GSASYA | | RDTST | | GGDDGG | | VTVSS | | GAGGTGAAGAAGCCTGGGGCCTCAGTG | | VKKPGASVKV | | | |
| | | SVKVSCKA | | | | EWMG | | QKFQG | | STVYM | | WFDP | | | | AAAGTTTCCTGCAAGGCATCTGGATAC | | SCKASGYIFI | | | |
| | | SG | | | | | | | | EVTSL | | | | | | ATCTTCATCAACTACTATATAAGCTGG | | NYYISWVRQA | | | |
| | | | | | | | | | | QSEDT | | | | | | GTGCGACAGGCCCCTGGACAAGGGCTT | | PGQGLEWMGI | | | |
| | | | | | | | | | | AVYYC | | | | | | GAGTGGATGGGAATAATCAGCCCTAGA | | ISPRSGSASY | | | |
| | | | | | | | | | | | | | | | | AGTGGTAGCGCGAGCTACGCACAGAAG | | AQKFQGRVTM | | | |
| | | | | | | | | | | | | | | | | TTCCAGGGCAGAGTCACCATGAGTAGG | | SRDTSTSTVY | | | |
| | | | | | | | | | | | | | | | | GACACGTCCACGAGTACAGTCTACATG | | MEVTSLQSED | | | |
| | | | | | | | | | | | | | | | | GAGGTGACCAGCCTGCAATCTGAGGAC | | TAVYYCARDR | | | |
| | | | | | | | | | | | | | | | | ACGGCCGTTTATTACTGTGCGAGAGAT | | RSGGDDGGWF | | | |
| | | | | | | | | | | | | | | | | CGGCGATCCGGTGGTGACGATGGGGGC | | DPWGQGTLVT | | | |
| | | | | | | | | | | | | | | | | TGGTTCGACCCCTGGGGCCAGGGAACC | | VSS | | | |
| | | | | | | | | | | | | | | | | CTGGTCACCGTCTCCTCA | | | | | |
| ADI- | IGHV3- | EVQLEQSG | 15 | FTFRRYG | 204 | WVRQA | 431 | VISYDG | 525 | RFTSS | 783 | AKQSGQ | 1027 | WGQGAL | 1285 | GAGGTGCAGCTGGAGCAGTCTGGGGGA | 1312 | EVQLEQSGGG | 1595 | | |
| 75590 | 30 | GGVVQPGR | | MH | | PGKGL | | ENKDYP | | RDNSK | | YCSGGT | | VTVSS | | GGCGTAGTCCAGCCTGGGAGGTCCCTG | | VVQPGRSLRL | | | |
| | | SLRLSCAA | | | | EWVA | | DSVKG | | NTLYL | | CYEGSL | | | | AGACTCTCCTGTGCAGCCTCTGGATTC | | SCAASGFTFR | | | |
| | | SG | | | | | | | | EMNSL | | DY | | | | ACTTTCAGAAGGTATGGCATGCACTGG | | RYGMHWVRQA | | | |
| | | | | | | | | | | RAEDT | | | | | | GTCCGCCAGGCTCCAGGCAAGGGTCTG | | PGKGLEWVAV | | | |
| | | | | | | | | | | AVYYC | | | | | | GAGTGGGTGGCAGTTATATCATATGAT | | ISYDGENKDY | | | |
| | | | | | | | | | | | | | | | | GGAGAAAACAAAGACTATCCGGACTCC | | PDSVKGRFTS | | | |
| | | | | | | | | | | | | | | | | GTGAAGGGCCGATTCACCAGCTCCAGA | | SRDNSKNTLY | | | |
| | | | | | | | | | | | | | | | | GACAATTCCAAGAACACGCTGTATCTG | | LEMNSLRAED | | | |
| | | | | | | | | | | | | | | | | GAAATGAACAGCCTGAGAGCTGAGGAC | | TAVYYCAKQS | | | |
| | | | | | | | | | | | | | | | | ACGGCTGTCTATTACTGTGCGAAACAG | | GQYCSGGTCY | | | |
| | | | | | | | | | | | | | | | | TCCGGTCAATATTGTAGTGGTGGTACC | | EGSLDYWGQG | | | |
| | | | | | | | | | | | | | | | | TGCTACGAAGGGTCCCTTGACTACTGG | | ALVTVSS | | | |
| | | | | | | | | | | | | | | | | GGCCAGGGAGCCCTGGTCACCGTCTCC | | | | | |
| | | | | | | | | | | | | | | | | TCA | | | | | |
| ADI- | IGHV3- | QVQLVESG | 11 | FTFSNYA | 205 | WVRQA | 431 | VILKDG | 526 | RFTIS | 784 | ARDYGG | 1028 | WGQGTL | 1286 | CAGGTGCAGCTGGTGGAGTCTGGGGGA | 1313 | QVQLVESGGG | 1596 | | |
| 75823 | 30 | GGVVQPGR | | IH | | PGKGL | | SGKFYA | | RDNSK | | YVTYFD | | VTVSS | | GGCGTGGTCCAGCCTGGGAGGTCCCTG | | VVQPGRSLRL | | | |
| | | SLRLSCAA | | | | EWVA | | DSVKG | | NTVFL | | Y | | | | AGACTCTCCTGTGCAGCCTCTGGATTC | | SCAASGFTFS | | | |
| | | SG | | | | | | | | QTNSL | | | | | | ACCTTCAGTAATTATGCTATACATTGG | | NYAIHWVRQA | | | |
| | | | | | | | | | | RPEDT | | | | | | GTCCGCCAGGCTCCAGGCAAGGGGCTG | | PGKGLEWVAV | | | |
| | | | | | | | | | | AMYYC | | | | | | GAGTGGGTGGCAGTTATATTAAAAGAT | | ILKDGSGKFY | | | |
| | | | | | | | | | | | | | | | | GGAAGCGGGAAATTCTACGCAGACTCC | | ADSVKGRFTI | | | |
| | | | | | | | | | | | | | | | | GTGAAGGGCCGATTCACCATCTCCAGA | | SRDNSKNTVF | | | |
| | | | | | | | | | | | | | | | | GACAATTCCAAGAACACGGTGTTTCTG | | LQTNSLRPED | | | |
| | | | | | | | | | | | | | | | | CAAACGAACAGCCTGAGACCTGAGGAC | | TAMYYCARDY | | | |
| | | | | | | | | | | | | | | | | ACGGCTATGTATTACTGTGCGAGAGAC | | GGYVTYFDYW | | | |
| | | | | | | | | | | | | | | | | TACGGTGGCTACGTGACTTACTTTGAC | | GQGTLVTVSS | | | |
| | | | | | | | | | | | | | | | | TACTGGGGCCAGGGAACCCTGGTCACC | | | | | |
| | | | | | | | | | | | | | | | | GTCTCCTCA | | | | | |
| ADI- | IGHV4- | QVQLQQWG | 16 | GSFSGYY | 206 | WIRQS | 434 | EINHSG | 527 | RVIIS | 785 | ARARLG | 1029 | WGRGTL | 1288 | CAGGTGCAGCTACAGCAGTGGGGCGCA | 1314 | QVQLQQWGAG | 1597 | | |
| 75569 | 34 | AGLLKPSD | | WS | | PGKGL | | STNYNP | | VDTSK | | ESYWHF | | VTVSS | | GGACTGTTGAAGCCTTCGGATACCCTG | | LLKPSDTLSR | | | |
| | | TLSRTCAV | | | | EWIG | | SLKS | | KQFSL | | DL | | | | TCCCGCACCTGCGCTGTCTATGGTGGG | | TCAVYGGSFS | | | |
| | | YG | | | | | | | | NLTSV | | | | | | TCCTTCAGTGGTTACTACTGGAGTTGG | | GYYWSWIRQS | | | |
| | | | | | | | | | | TAADT | | | | | | ATCCGCCAGTCCCCAGGGAAGGGGCTG | | PGKGLEWIGE | | | |
| | | | | | | | | | | AIYYC | | | | | | GAGTGGATTGGAGAAATCAATCATAGT | | INHSGSTNYN | | | |
| | | | | | | | | | | | | | | | | GGAAGCACCAACTACAACCCGTCCCTC | | PSLKSRVIIS | | | |
| | | | | | | | | | | | | | | | | AAGAGTCGAGTCATCATATCAGTGGAC | | VDTSKKQFSL | | | |
| | | | | | | | | | | | | | | | | ACGTCCAAGAAGCAGTTCTCCCTGAAC | | NLTSVTAADT | | | |
| | | | | | | | | | | | | | | | | CTGACCTCTGTGACCGCCGCGGACACG | | AIYYCARARL | | | |
| | | | | | | | | | | | | | | | | GCTATATATTACTGTGCGAGAGCGCGG | | GESYWHFDLW | | | |
| | | | | | | | | | | | | | | | | TTGGGCGAAAGTTACTGGCACTTCGAT | | GRGTLVTVSS | | | |
| | | | | | | | | | | | | | | | | CTCTGGGGCCGTGGCACCCTGGTCACC | | | | | |
| | | | | | | | | | | | | | | | | GTCTCCTCA | | | | | |
| ADI- | IGHV3- | QVQLVQSG | 12 | FTFSSYD | 207 | WVRQG | 435 | TIGTAG | 528 | RFTIS | 786 | ARQGNG | 1030 | WGRGTL | 1288 | CAGGTCCAGCTGGTGCAGTCTGGGGGA | 1315 | QVQLVQSGGG | 1598 | | |
| 75623 | 13 | GGLVQPGG | | MH | | TGNRL | | DTYYAD | | RENAK | | YTYGNW | | VTVSS | | GGCTTGGTACAGCCTGGGGGGTCCCTG | | LVQPGGSLRL | | | |
| | | SLRLSCAA | | | | EWVS | | SVKG | | NSLYL | | YFDL | | | | AGACTCTCCTGTGCAGCCTCTGGATTC | | SCAASGFTFS | | | |
| | | SG | | | | | | | | QMSSL | | | | | | ACCTTCAGTTCCTACGACATGCACTGG | | SYDMHWVRQG | | | |
| | | | | | | | | | | TAGDT | | | | | | GTCCGCCAAGGTACAGGGAACCGTCTG | | TGNRLEWVST | | | |
| | | | | | | | | | | AVYYC | | | | | | GAGTGGGTCTCAACAATTGGTACTGCT | | IGTAGDTYYA | | | |
| | | | | | | | | | | | | | | | | GGCGACACATACTATGCAGACTCCGTG | | DSVKGRFTIS | | | |
| | | | | | | | | | | | | | | | | AAGGGCCGATTCACCATCTCCAGAGAA | | RENAKNSLYL | | | |
| | | | | | | | | | | | | | | | | AATGCCAAGAACTCCTTGTATCTTCAA | | QMSSLTAGDT | | | |
| | | | | | | | | | | | | | | | | ATGAGCAGCCTGACAGCCGGAGACACG | | AVYYCARQGN | | | |
| | | | | | | | | | | | | | | | | GCTGTCTATTACTGTGCAAGACAAGGC | | GYTYGNWYFD | | | |
| | | | | | | | | | | | | | | | | AATGGATACACCTATGGCAACTGGTAC | | LWGRGTLVTV | | | |
| | | | | | | | | | | | | | | | | TTCGATCTCTGGGGCCGTGGCACCCTG | | SS | | | |
| | | | | | | | | | | | | | | | | GTCACCGTCTCCTCA | | | | | |
| ADI- | IGHV3- | QVQLVQSG | 17 | FTFEDYA | 208 | WVRQV | 436 | GISYNS | 529 | RFTIS | 787 | AKSLRE | 1031 | WGQGTT | 1287 | CAGGTGCAGCTGGTGCAGTCTGGGGGA | 1316 | QVQLVQSGGG | 1599 | | |
| 75626 | 9 | GGLVQPGR | | MH | | PGKGL | | GSVDYA | | RDNAK | | FHSYYY | | VTVSS | | GGCTTGGTACAGCCTGGCAGGTCCCTG | | LVQPGRSLRL | | | |
| | | SLRLSCAA | | | | EWVS | | DSVKG | | NSLYL | | GMDV | | | | AGACTCTCTTGTGCAGCCTCTGGATTC | | SCAASGFTFE | | | |
| | | SG | | | | | | | | QMNSL | | | | | | ACCTTTGAGGATTATGCCATGCACTGG | | DYAMHWVRQV | | | |
| | | | | | | | | | | KNEDT | | | | | | GTCCGCCAAGTTCCAGGGAAGGGCCTG | | PGKGLEWVSG | | | |
| | | | | | | | | | | ALYYC | | | | | | GAGTGGGTCTCAGGTATCAGTTACAAT | | ISYNSGSVDY | | | |
| | | | | | | | | | | | | | | | | AGTGGTAGCGTAGACTATGCGGACTCT | | ADSVKGRFTI | | | |
| | | | | | | | | | | | | | | | | GTGAAGGGCCGATTCACCATCTCCAGA | | SRDNAKNSLY | | | |
| | | | | | | | | | | | | | | | | GACAACGCCAAGAACTCCCTGTATCTG | | LQMNSLKNED | | | |
| | | | | | | | | | | | | | | | | CAAATGAACAGTCTGAAAAATGAGGAC | | TALYYCAKSL | | | |
| | | | | | | | | | | | | | | | | ACGGCCTTGTATTATTGTGCAAAAAGT | | REFHSYYYGM | | | |
| | | | | | | | | | | | | | | | | CTTAGGGAGTTCCACTCCTACTACTAC | | DVWGQGTTVT | | | |
| | | | | | | | | | | | | | | | | GGCATGGACGTCTGGGGCCAAGGGACC | | VSS | | | |
| | | | | | | | | | | | | | | | | ACGGTCACCGTCTCCTCA | | | | | |
| ADI- | IGHV3- | QVQLVQSG | 18 | LPFSSNA | 209 | WVRQA | 437 | GISGRG | 530 | RFTVS | 788 | ATDQVG | 1032 | WGQGTL | 1286 | CAGGTGCAGCTGGTGCAGTCTGGGGGA | 1317 | QVQLVQSGGG | 1600 | | |
| 75622 | 23 | GGLVQPGG | | MS | | PGKGL | | ETTYYV | | RDNSK | | GFTMVQ | | VTVSS | | GGCTTGGTACAGCCGGGGGGGTCCCTG | | LVQPGGSLRL | | | |
| | | SLRLSCAV | | | | EWVS | | DSVKG | | NTLFL | | PEYFHH | | | | AGACTCTCCTGTGCAGTCTCTGGACTC | | SCAVSGLPFS | | | |
| | | SG | | | | | | | | QMDSL | | | | | | CCCTTTAGTAGCAATGCCATGAGCTGG | | SNAMSWVRQA | | | |
| | | | | | | | | | | RAEDT | | | | | | GTCCGCCAGGCTCCAGGGAAGGGGCTG | | PGKGLEWVSG | | | |
| | | | | | | | | | | AVYYC | | | | | | GAGTGGGTCTCAGGTATTAGTGGTAGA | | ISGRGETTYY | | | |
| | | | | | | | | | | | | | | | | GGTGAGACCACATACTACGTAGACTCC | | VDSVKGRFTV | | | |
| | | | | | | | | | | | | | | | | GTGAAGGGCCGGTTCACCGTCTCCAGA | | SRDNSKNTLF | | | |
| | | | | | | | | | | | | | | | | GACAATTCGAAGAACACGCTGTTTTTG | | LQMDSLRAED | | | |
| | | | | | | | | | | | | | | | | CAAATGGACAGCCTGAGAGCCGAGGAC | | TAVYYCATDQ | | | |
| | | | | | | | | | | | | | | | | ACGGCTGTATACTACTGTGCGACAGAT | | VGGFTMVQPE | | | |
| | | | | | | | | | | | | | | | | CAAGTAGGGGGATTTACTATGGTTCAG | | YFHHWGQGTL | | | |
| | | | | | | | | | | | | | | | | CCAGAGTACTTCCACCACTGGGGCCAG | | VTVSS | | | |
| | | | | | | | | | | | | | | | | GGCACCCTGGTCACCGTCTCCTCA | | | | | |
| ADI- | IGHV3- | QVQLEQSG | 19 | FTFKNYG | 210 | WVRQA | 431 | VILYDR | 531 | RFTIS | 789 | AKGGAP | 1033 | WGQGSL | 1289 | CAGGTCCAGCTGGAGCAGTCTGGGGGA | 1318 | QVQLEQSGGG | 1601 | | |
| 75598 | 30 | GGVVQPGR | | MH | | PGKGL | | SGKHYA | | RDNSK | | YCGGSN | | VTVSS | | GGCGTGGTCCAGCCTGGGAGGTCCCTG | | VVQPGRSLRL | | | |
| | | SLRLSCAA | | | | EWVA | | DSVKG | | NTLYL | | CYLGYF | | | | AGACTCTCCTGTGCAGCCTCTGGATTC | | SCAASGFTFK | | | |
| | | SG | | | | | | | | QMNSL | | DY | | | | ACTTTCAAGAACTATGGCATGCACTGG | | NYGMHWVRQA | | | |
| | | | | | | | | | | RTEDT | | | | | | GTCCGCCAGGCTCCAGGCAAGGGGCTG | | PGKGLEWVAV | | | |
| | | | | | | | | | | AVYYC | | | | | | GAGTGGGTGGCAGTTATATTATATGAC | | ILYDRSGKHY | | | |
| | | | | | | | | | | | | | | | | CGAAGTGGTAAACACTACGCAGACTCT | | ADSVKGRFTI | | | |
| | | | | | | | | | | | | | | | | GTGAAGGGCCGATTCACCATCTCCAGA | | SRDNSKNTLY | | | |
| | | | | | | | | | | | | | | | | GACAATTCCAAGAACACTCTCTATCTG | | LQMNSLRTED | | | |
| | | | | | | | | | | | | | | | | CAAATGAACAGCCTGAGAACTGAAGAC | | TAVYYCAKGG | | | |
| | | | | | | | | | | | | | | | | ACGGCTGTGTACTACTGTGCGAAAGGT | | APYCGGSNCY | | | |
| | | | | | | | | | | | | | | | | GGGGCCCCATATTGTGGTGGTAGTAAC | | LGYFDYWGQG | | | |
| | | | | | | | | | | | | | | | | TGCTACCTGGGCTACTTTGACTACTGG | | SLVTVSS | | | |
| | | | | | | | | | | | | | | | | GGCCAGGGATCCCTGGTCACCGTCTCC | | | | | |
| | | | | | | | | | | | | | | | | TCA | | | | | |
| ADI- | IGHV3- | QVQLQQSG | 20 | FTFNNHA | 211 | WVRQA | 431 | VVSYDG | 532 | RFTIS | 790 | AREDYH | 1034 | WGQGTL | 1286 | CAGGTGCAGCTGCAGCAGTCTGGGGGA | 1319 | QVQLQQSGGG | 1602 | | |
| 75572 | 30 | GGVVQPGR | | MH | | PGKGL | | SNKYYA | | RDNSK | | DSSGSF | | VTVSS | | GGCGTGGTCCAGCCTGGGAGGTCCCTG | | VVQPGRSLRL | | | |
| | | SLRLSCAA | | | | EWVA | | NSVKG | | KMVYL | | DY | | | | AGACTCTCCTGTGCAGCCTCTGGATTC | | SCAASGFTFN | | | |
| | | SG | | | | | | | | QMNSL | | | | | | ACCTTCAATAACCATGCTATGCACTGG | | NHAMHWVRQA | | | |
| | | | | | | | | | | RPEDS | | | | | | GTCCGCCAGGCTCCAGGCAAGGGGCTG | | PGKGLEWVAV | | | |
| | | | | | | | | | | AVFYC | | | | | | GAGTGGGTGGCAGTTGTTTCATATGAT | | VSYDGSNKYY | | | |
| | | | | | | | | | | | | | | | | GGAAGTAACAAATACTACGCAAACTCC | | ANSVKGRFTI | | | |
| | | | | | | | | | | | | | | | | GTGAAGGGCCGATTCACCATCTCCAGA | | SRDNSKKMVY | | | |
| | | | | | | | | | | | | | | | | GACAATTCCAAGAAAATGGTGTATTTG | | LQMNSLRPED | | | |
| | | | | | | | | | | | | | | | | CAAATGAACAGCCTGAGACCTGAGGAC | | SAVFYCARED | | | |
| | | | | | | | | | | | | | | | | TCGGCTGTGTTTTACTGTGCGAGAGAG | | YHDSSGSFDY | | | |
| | | | | | | | | | | | | | | | | GATTACCATGATAGTAGTGGATCCTTT | | WGQGTLVTVS | | | |
| | | | | | | | | | | | | | | | | GACTACTGGGGCCAGGGAACCCTGGTC | | S | | | |
| | | | | | | | | | | | | | | | | ACCGTCTCCTCA | | | | | |
| ADI- | IGHV4- | QVQLQESG | 21 | AALSSSN | 212 | WVRQS | 438 | EIFHSG | 533 | RVTIS | 791 | ARVAGM | 1035 | WGQGTL | 1286 | CAGGTGCAGCTGCAGGAGTCGGGGCCA | 1320 | QVQLQESGPG | 1603 | | |
| 75819 | 4 | PGLVKPSG | | WWS | | PGKGL | | STYYNP | | VDKSK | | FDY | | VTVSS | | GGACTGGTGAAGCCTTCGGGGACCCTG | | LVKPSGTLSL | | | |
| | | TLSLTCAV | | | | EWIG | | SLKS | | SQFSL | | | | | | TCCCTCACCTGCGCTGTCTCTGGTGCC | | TCAVSGAALS | | | |
| | | SG | | | | | | | | KLTSV | | | | | | GCCCTCAGCAGTAGTAACTGGTGGAGT | | SSNWWSWVRQ | | | |
| | | | | | | | | | | TAADT | | | | | | TGGGTCCGCCAGTCCCCAGGGAAGGGG | | SPGKGLEWIG | | | |
| | | | | | | | | | | AVYYC | | | | | | CTGGAGTGGATTGGGGAAATCTTTCAT | | EIFHSGSTYY | | | |
| | | | | | | | | | | | | | | | | AGTGGGAGCACCTACTACAATCCGTCC | | NPSLKSRVTI | | | |
| | | | | | | | | | | | | | | | | CTCAAGAGTCGAGTCACCATATCAGTA | | SVDKSKSQFS | | | |
| | | | | | | | | | | | | | | | | GACAAGTCGAAGAGCCAATTCTCCCTG | | LKLTSVTAAD | | | |
| | | | | | | | | | | | | | | | | AAGCTGACCTCTGTGACCGCCGCGGAC | | TAVYYCARVA | | | |
| | | | | | | | | | | | | | | | | ACGGCCGTGTATTACTGTGCGAGAGTC | | GMFDYWGQGT | | | |
| | | | | | | | | | | | | | | | | GCTGGGATGTTTGACTACTGGGGCCAG | | LVTVSS | | | |
| | | | | | | | | | | | | | | | | GGAACCCTGGTCACCGTCTCCTCA | | | | | |
| ADI- | IGHV3- | QVQLQQSG | 22 | FTFRTYG | 213 | WVRQA | 431 | VVLYDG | 534 | RFTIS | 792 | AKQSGQ | 1027 | WGQGAL | 1285 | CAGGTGCAGCTGCAGCAGTCTGGGGGA | 1321 | QVQLQQSGGG | 1604 | | |
| 75616 | 30 | GGVVQPGR | | MH | | PGKGL | | SNEDYA | | RDNSK | | YCSGGT | | VTVSS | | GGCGTGGTCCAGCCTGGGAGGTCCCTG | | VVQPGRSLRL | | | |
| | | SLRLSCEA | | | | EWVA | | DSVKG | | NTLYL | | CYEGSL | | | | AGACTCTCCTGTGAAGCCTCTGGATTC | | SCEASGFTFR | | | |
| | | SG | | | | | | | | QMNSL | | DY | | | | ACCTTCAGGACCTATGGCATGCATTGG | | TYGMHWVRQA | | | |
| | | | | | | | | | | RPEDT | | | | | | GTCCGCCAGGCTCCAGGCAAGGGGCTG | | PGKGLEWVAV | | | |
| | | | | | | | | | | AVYFC | | | | | | GAGTGGGTGGCAGTTGTATTATATGAT | | VLYDGSNEDY | | | |
| | | | | | | | | | | | | | | | | GGAAGTAATGAAGACTACGCAGATTCC | | ADSVKGRFTI | | | |
| | | | | | | | | | | | | | | | | GTGAAGGGCCGATTCACCATCTCCAGA | | SRDNSKNTLY | | | |
| | | | | | | | | | | | | | | | | GACAATTCCAAGAACACGCTGTATCTG | | LQMNSLRPED | | | |
| | | | | | | | | | | | | | | | | CAGATGAACAGCCTGAGACCTGAGGAC | | TAVYFCAKQS | | | |
| | | | | | | | | | | | | | | | | ACGGCTGTGTATTTCTGTGCGAAGCAG | | GQYCSGGTCY | | | |
| | | | | | | | | | | | | | | | | TCCGGTCAATATTGTAGTGGTGGTACT | | EGSLDYWGQG | | | |
| | | | | | | | | | | | | | | | | TGCTACGAAGGGTCCCTTGACTACTGG | | ALVTVSS | | | |
| | | | | | | | | | | | | | | | | GGCCAGGGAGCCCTGGTCACCGTCTCC | | | | | |
| | | | | | | | | | | | | | | | | TCA | | | | | |
| ADI- | IGHV3- | QVQLVQSG | 23 | FTFDDYA | 214 | WVRQA | 437 | GISWNG | 535 | RFIIS | 793 | AKGFGG | 1036 | WGQGAL | 1285 | CAGGTGCAGCTGGTGCAGTCTGGGGGA | 1322 | QVQLVQSGGG | 1605 | | |
| 75581 | 9 | GGLVQPGR | | MH | | PGKGL | | GVLDYA | | RDNVS | | IGADRL | | VTVSS | | GGCTTGGTACAGCCTGGCAGGTCCCTG | | LVQPGRSLRL | | | |
| | | SLRLSCEA | | | | EWVS | | DSVKG | | RDNVK | | TGSEEY | | | | AGACTCTCCTGTGAAGCCTCTGGATTC | | SCEASGFTFD | | | |
| | | SG | | | | | | | | NSLYL | | YFDY | | | | ACCTTTGATGATTATGCCATGCACTGG | | DYAMHWVRQA | | | |
| | | | | | | | | | | QMNSL | | | | | | GTCCGGCAAGCTCCAGGGAAGGGCCTG | | PGKGLEWVSG | | | |
| | | | | | | | | | | RAEDT | | | | | | GAGTGGGTCTCAGGTATTAGTTGGAAT | | ISWNGGVLDY | | | |
| | | | | | | | | | | ALYYC | | | | | | GGTGGTGTATTAGACTATGCGGACTCT | | ADSVKGRFII | | | |
| | | | | | | | | | | | | | | | | GTGAAGGGCCGATTCATCATCTCCAGA | | SRDNVSRDNV | | | |
| | | | | | | | | | | | | | | | | GACAATGTCTCCAGAGACAACGTCAAG | | KNSLYLQMNS | | | |
| | | | | | | | | | | | | | | | | AACTCTCTGTATCTGCAAATGAACAGT | | LRAEDTALYY | | | |
| | | | | | | | | | | | | | | | | CTGAGAGCTGAGGACACGGCCTTATAT | | CAKGFGGIGA | | | |
| | | | | | | | | | | | | | | | | TACTGTGCAAAAGGTTTTGGAGGCATA | | DRLTGSEEYY | | | |
| | | | | | | | | | | | | | | | | GGAGCAGATCGGCTTACGGGGAGTGAG | | FDYWGQGALV | | | |
| | | | | | | | | | | | | | | | | GAGTACTACTTTGACTACTGGGGCCAG | | TVSS | | | |
| | | | | | | | | | | | | | | | | GGAGCCCTGGTCACCGTCTCCTCA | | | | | |
| ADI- | IGHV3- | QVQLQQSG | 20 | FTFSTYG | 215 | WVRQA | 431 | VIAFDG | 536 | RFTIS | 794 | AKNQAP | 1037 | WGQGTL | 1286 | CAGGTGCAGCTGCAGCAGTCTGGGGGA | 1323 | QVQLQQSGGG | 1606 | | |
| 75613 | 30 | GGVVQPGR | | MH | | PGKGL | | SKKYYG | | RDSSK | | YCGGGN | | VTVSS | | GGCGTGGTCCAGCCTGGGAGGTCCCTG | | VVQPGRSLRL | | | |
| | | SLRLSCAA | | | | EWVA | | DSVKG | | NTLYL | | CLNGYF | | | | AGACTCTCCTGTGCAGCGTCTGGATTC | | SCAASGFTFS | | | |
| | | SG | | | | | | | | QMNSL | | DY | | | | ACCTTCAGTACCTATGGCATGCACTGG | | TYGMHWVRQA | | | |
| | | | | | | | | | | RPEDT | | | | | | GTCCGCCAGGCTCCAGGCAAGGGGCTG | | PGKGLEWVAV | | | |
| | | | | | | | | | | AVYYC | | | | | | GAGTGGGTGGCAGTTATAGCATTTGAT | | IAFDGSKKYY | | | |
| | | | | | | | | | | | | | | | | GGAAGTAAGAAATATTATGGAGACTCC | | GDSVKGRFTI | | | |
| | | | | | | | | | | | | | | | | GTGAAGGGCCGATTCACCATCTCCAGA | | SRDSSKNTLY | | | |
| | | | | | | | | | | | | | | | | GACAGCTCCAAGAACACGCTGTATCTG | | LQMNSLRPED | | | |
| | | | | | | | | | | | | | | | | CAAATGAACAGCCTGAGACCTGAGGAC | | TAVYYCAKNQ | | | |
| | | | | | | | | | | | | | | | | ACGGCTGTCTATTACTGTGCGAAGAAC | | APYCGGGNCL | | | |
| | | | | | | | | | | | | | | | | CAAGCCCCGTATTGTGGTGGTGGGAAC | | NGYFDYWGQG | | | |
| | | | | | | | | | | | | | | | | TGCCTCAACGGCTACTTTGACTACTGG | | TLVTVSS | | | |
| | | | | | | | | | | | | | | | | GGCCAGGGAACCCTGGTCACCGTCTCC | | | | | |
| | | | | | | | | | | | | | | | | TCA | | | | | |
| ADI- | IGHV3- | QVQLVESG | 24 | FTFSSYD | 207 | WVRQG | 439 | VIGTGG | 537 | RFTIS | 795 | ARVTHI | 1038 | WGRGTL | 1288 | CAGGTGCAGCTGGTGGAGTCTGGGGGA | 1324 | QVQLVESGGG | 1607 | | |
| 75872 | 13 | GGLVQPGG | | MH | | IGKGL | | DTYYPD | | RDNGK | | TMGLKM | | VTVSS | | GGCTTGGTACAGCCTGGGGGGTCCCTG | | LVQPGGSLRL | | | |
| | | SLRLSCAA | | | | EWVS | | SVKG | | NSLYL | | YWYLDL | | | | AGACTCTCCTGTGCAGCCTCTGGATTC | | SCAASGFTFS | | | |
| | | SG | | | | | | | | QMNSL | | | | | | ACCTTCAGTAGCTACGACATGCACTGG | | SYDMHWVRQG | | | |
| | | | | | | | | | | RAGDT | | | | | | GTCCGCCAAGGTATAGGAAAAGGTCTG | | IGKGLEWVSV | | | |
| | | | | | | | | | | AVYYC | | | | | | GAGTGGGTCTCAGTTATCGGTACTGGT | | IGTGGDTYYP | | | |
| | | | | | | | | | | | | | | | | GGTGACACATACTATCCAGACTCCGTG | | DSVKGRFTIS | | | |
| | | | | | | | | | | | | | | | | AAGGGCCGCTTCACAATCTCCAGAGAC | | RDNGKNSLYL | | | |
| | | | | | | | | | | | | | | | | AATGGCAAGAACTCCTTGTATCTTCAA | | QMNSLRAGDT | | | |
| | | | | | | | | | | | | | | | | ATGAACAGCCTGAGAGCCGGGGACACG | | AVYYCARVTH | | | |
| | | | | | | | | | | | | | | | | GCTGTGTATTACTGTGCAAGAGTCACG | | ITMGLKMYWY | | | |
| | | | | | | | | | | | | | | | | CATATAACAATGGGGCTGAAGATGTAC | | LDLWGRGTLV | | | |
| | | | | | | | | | | | | | | | | TGGTACCTCGATCTCTGGGGCCGTGGC | | TVSS | | | |
| | | | | | | | | | | | | | | | | ACCCTGGTCACCGTCTCCTCA | | | | | |
| VYD223 | IGHV4- | EVQLQQSG | 25 | ASISNHD | 216 | WVRQS | 438 | AIFHSG | 538 | RLTIS | 796 | ARARGI | 1039 | WGQGTL | 1286 | GAGGTGCAGCTGCAGCAGTCAGGCCCA | 1325 | EVQLQQSGPG | 1608 | QVQLQESG | 1872 |
| (ADI- | 4 | PGLVKPSG | | WWT | | PGKGL | | TTNYNP | | VDKSK | | LDF | | VTVSS | | GGACTGGTGAAGCCTTCGGGGACCCTG | | LVKPSGTLSL | | PGLVKPSG | |
| 75865) | | TLSLTCAV | | | | EWIG | | SLRS | | SHFSL | | | | | | TCCCTCACCTGCGCTGTGTCTGGTGCC | | TCAVSGASIS | | TLSLTCAV | |
| | | SG | | | | | | | | KLTSV | | | | | | TCCATCAGCAATCATGACTGGTGGACT | | NHDWWTWVRQ | | SGASISNH | |
| | | | | | | | | | | TAADT | | | | | | TGGGTCCGCCAGTCCCCAGGGAAGGGG | | SPGKGLEWIG | | DWWTWVRQ | |
| | | | | | | | | | | AVYYC | | | | | | CTGGAATGGATTGGCGCAATATTTCAT | | AIFHSGTTNY | | SPGKGLEW | |
| | | | | | | | | | | | | | | | | AGTGGGACCACCAACTACAACCCGTCC | | NPSLRSRLTI | | IGAIFHSG | |
| | | | | | | | | | | | | | | | | CTCAGGAGTCGACTCACCATATCAGTA | | SVDKSKSHFS | | TTNYNPSL | |
| | | | | | | | | | | | | | | | | GACAAGTCCAAGAGCCACTTCTCCCTG | | LKLTSVTAAD | | RSRLTISV | |
| | | | | | | | | | | | | | | | | AAACTGACCTCTGTGACCGCCGCGGAC | | TAVYYCARAR | | DKSKSHFS | |
| | | | | | | | | | | | | | | | | ACGGCCGTCTATTACTGTGCGAGAGCC | | GILDFWGQGT | | LKLTSVTA | |
| | | | | | | | | | | | | | | | | CGAGGAATCCTTGACTTTTGGGGCCAG | | LVTVSS | | ADTAVYYC | |
| | | | | | | | | | | | | | | | | GGAACCCTGGTCACCGTCTCCTCA | | | | ARARGILD | |
| | | | | | | | | | | | | | | | | | | | | FWGQGTLV | |
| | | | | | | | | | | | | | | | | | | | | TVSS | |
| ADI- | IGHV5- | SLRISCKG | 26 | YTFTNYW | 217 | WVRQI | 440 | KIDPSD | 539 | HVTIS | 797 | ARQKKY | 1040 | WGQGTL | 1286 | CAGGTGCAGCTGCAGGAGTCCGGAGCA | 1326 | QVQLQESGAE | 1609 | | |
| 75822 | 10-1 | QVQLQESG | | IS | | PGKGL | | SYTDYN | | ADKSS | | SSSPFD | | VTVSS | | GAGGTGAAAAAGCCCGGGGAGTCTCTG | | VKKPGESLRI | | | |
| | | AEVKKPGE | | | | EWMG | | PSFEG | | STAYL | | P | | | | AGGATCTCCTGTAAGGGTTCTGGATAC | | SCKGSGYTFT | | | |
| | | SG | | | | | | | | QRTNL | | | | | | ACTTTTACCAACTACTGGATCAGCTGG | | NYWISWVRQI | | | |
| | | | | | | | | | | EASDS | | | | | | GTGCGCCAGATACCCGGGAAAGGCCTG | | PGKGLEWMGK | | | |
| | | | | | | | | | | AIYYC | | | | | | GAGTGGATGGGGAAAATTGATCCTAGT | | IDPSDSYTDY | | | |
| | | | | | | | | | | | | | | | | GACTCTTATACCGACTACAACCCGTCC | | NPSFEGHVTI | | | |
| | | | | | | | | | | | | | | | | TTCGAAGGCCACGTCACCATCTCAGCT | | SADKSSSTAY | | | |
| | | | | | | | | | | | | | | | | GACAAGTCCTCCAGCACTGCCTACCTG | | LQRTNLEASD | | | |
| | | | | | | | | | | | | | | | | CAGAGGACCAACCTGGAGGCCTCGGAC | | SAIYYCARQK | | | |
| | | | | | | | | | | | | | | | | AGCGCCATTTATTACTGTGCGAGACAG | | KYSSSPFDPW | | | |
| | | | | | | | | | | | | | | | | AAGAAATATAGCAGTAGTCCGTTCGAC | | GQGTLVTVSS | | | |
| | | | | | | | | | | | | | | | | CCCTGGGGCCAAGGAACCCTGGTCACC | | | | | |
| | | | | | | | | | | | | | | | | GTCTCCTCA | | | | | |
| ADI- | IGHV4- | QVQLQQSG | 27 | GSITGSD | 218 | WVRQS | 438 | EIFHSG | 540 | RVSFS | 798 | ARTRGL | 1041 | WGQGTL | 1286 | CAGGTGCAGCTGCAGCAGTCGGGCCCA | 1327 | QVQLQQSGPR | 1610 | | |
| 75843 | 4 | PRLVKPSG | | WWT | | PGKGL | | SAVYNP | | VDKSK | | IDF | | VTVSS | | AGACTGGTGAAGCCTTCGGGGACCCTG | | LVKPSGTLSL | | | |
| | | TLSLTCAV | | | | EWIG | | SLES | | NHFSL | | | | | | TCCCTCACCTGCGCTGTCTCTGGTGGC | | TCAVSGGSIT | | | |
| | | SG | | | | | | | | NLRSV | | | | | | TCCATCACCGGTAGTGACTGGTGGACT | | GSDWWTWVRQ | | | |
| | | | | | | | | | | TAADT | | | | | | TGGGTCCGCCAGTCCCCAGGGAAGGGA | | SPGKGLEWIG | | | |
| | | | | | | | | | | AVYYC | | | | | | CTGGAGTGGATTGGGGAAATCTTTCAT | | EIFHSGSAVY | | | |
| | | | | | | | | | | | | | | | | AGTGGGAGCGCCGTCTACAACCCGTCC | | NPSLESRVSF | | | |
| | | | | | | | | | | | | | | | | CTCGAGAGTCGAGTCAGCTTTTCAGTA | | SVDKSKNHFS | | | |
| | | | | | | | | | | | | | | | | GACAAGTCCAAAAACCACTTCTCCCTG | | LNLRSVTAAD | | | |
| | | | | | | | | | | | | | | | | AACCTGAGGTCTGTGACCGCCGCGGAC | | TAVYYCARTR | | | |
| | | | | | | | | | | | | | | | | ACGGCCGTGTATTACTGTGCCCGTACA | | GLIDFWGQGT | | | |
| | | | | | | | | | | | | | | | | CGGGGCCTTATTGACTTCTGGGGCCAG | | LVTVSS | | | |
| | | | | | | | | | | | | | | | | GGAACCCTGGTCACCGTCTCCTCA | | | | | |
| ADI- | IGHV2- | QVPLVQSG | 28 | FSLSTSG | 219 | WIRQP | 441 | LIYWDD | 541 | RLTIT | 799 | AQRRGT | 1042 | WGQGTL | 1286 | CAGGTCCCGCTGGTGCAGTCTGGTCCT | 1328 | QVPLVQSGPT | 1611 | | |
| 75584 | 5 | PTLVKPTQ | | VGVG | | PGKAL | | DERYNP | | KDTSK | | MIVAEG | | VTVSS | | ACGCTGGTGAAACCCACACAGACCCTC | | LVKPTQTLTL | | | |
| | | TLTLTCTF | | | | EWLA | | SLKS | | SQVVL | | SFDS | | | | ACGCTGACCTGCACCTTCTCTGGGTTC | | TCTFSGFSLS | | | |
| | | SG | | | | | | | | TMTNM | | | | | | TCACTCAGCACTAGTGGAGTGGGTGTG | | TSGVGVGWIR | | | |
| | | | | | | | | | | GPVDT | | | | | | GGCTGGATCCGTCAGCCCCCAGGAAAG | | QPPGKALEWL | | | |
| | | | | | | | | | | GTYYC | | | | | | GCCCTGGAGTGGCTTGCACTCATTTAT | | ALIYWDDDER | | | |
| | | | | | | | | | | | | | | | | TGGGATGATGATGAGCGCTACAACCCA | | YNPSLKSRLT | | | |
| | | | | | | | | | | | | | | | | TCTCTGAAGAGCAGGCTCACCATCACC | | ITKDTSKSQV | | | |
| | | | | | | | | | | | | | | | | AAGGACACCTCCAAAAGCCAGGTGGTC | | VLTMTNMGPV | | | |
| | | | | | | | | | | | | | | | | CTTACAATGACCAACATGGGCCCTGTG | | DTGTYYCAQR | | | |
| | | | | | | | | | | | | | | | | GACACAGGCACATATTACTGTGCACAG | | RGTMIVAEGS | | | |
| | | | | | | | | | | | | | | | | AGACGGGGGACTATGATAGTGGCGGAG | | FDSWGQGTLV | | | |
| | | | | | | | | | | | | | | | | GGCTCCTTTGACTCCTGGGGCCAGGGA | | TVSS | | | |
| | | | | | | | | | | | | | | | | ACCCTGGTCACCGTCTCCTCA | | | | | |
| ADI- | IGHV3- | RCTLVQSG | 29 | FTFRHYG | 220 | WVRQA | 431 | VILYDG | 542 | RFTIS | 777 | AKQSGQ | 1043 | WGQGTL | 1286 | AGGTGCACGCTGGTGCAGTCTGGGGGA | 1329 | RCTLVQSGGG | 1612 | | |
| 75655 | 30 | GGVVQPGR | | MH | | PGKGL | | SNKDYA | | RDNSK | | YCSGGS | | VTVSS | | GGCGTGGTCCAGCCTGGGAGGTCCCTG | | VVQPGRSLRL | | | |
| | | SLRLSCAA | | | | EWVA | | DSVKG | | NTLYL | | CYEGSL | | | | AGACTCTCCTGTGCAGCCTCTGGATTC | | SCAASGFTFR | | | |
| | | SG | | | | | | | | QMNSL | | DY | | | | ACCTTCAGGCACTACGGCATGCACTGG | | HYGMHWVRQA | | | |
| | | | | | | | | | | RAEDT | | | | | | GTCCGCCAGGCACCAGGCAAGGGGCTG | | PGKGLEWVAV | | | |
| | | | | | | | | | | AVYYC | | | | | | GAGTGGGTGGCAGTTATATTATATGAT | | ILYDGSNKDY | | | |
| | | | | | | | | | | | | | | | | GGAAGTAATAAAGACTACGCAGACTCC | | ADSVKGRFTI | | | |
| | | | | | | | | | | | | | | | | GTGAAGGGCCGATTCACCATCTCCAGA | | SRDNSKNTLY | | | |
| | | | | | | | | | | | | | | | | GACAACTCCAAGAACACCCTGTATCTG | | LQMNSLRAED | | | |
| | | | | | | | | | | | | | | | | CAAATGAACAGCCTGAGAGCAGAGGAC | | TAVYYCAKQS | | | |
| | | | | | | | | | | | | | | | | ACGGCTGTGTATTACTGTGCGAAACAG | | GQYCSGGSCY | | | |
| | | | | | | | | | | | | | | | | TCCGGTCAATATTGTAGTGGTGGTAGT | | EGSLDYWGQG | | | |
| | | | | | | | | | | | | | | | | TGCTACGAAGGGTCCTTAGACTACTGG | | TLVTVSS | | | |
| | | | | | | | | | | | | | | | | GGCCAGGGAACCCTGGTCACCGTCTCC | | | | | |
| | | | | | | | | | | | | | | | | TCA | | | | | |
| ADI- | IGHV3- | QVQLVESG | 30 | FPFSTYD | 221 | WVRQA | 437 | GISDSG | 543 | RFTIS | 800 | AKLAGT | 1044 | WGQGTL | 1286 | CAGGTGCAGCTGGTGGAGTCTGGGGGA | 1330 | QVQLVESGGG | 1613 | | |
| 75641 | 23 | GGLIQPGG | | MG | | PGKGL | | GSPYYA | | RDNSN | | PPDYFD | | VTVSS | | GGCTTGATACAGCCGGGGGGGTCCCTG | | LIQPGGSLRL | | | |
| | | SLRLSCAA | | | | EWVS | | VSVRG | | NILHL | | Y | | | | AGACTCTCCTGTGCAGCCTCTGGATTT | | SCAASGFPFS | | | |
| | | SG | | | | | | | | QMNSL | | | | | | CCCTTTAGCACCTATGACATGGGCTGG | | TYDMGWVRQA | | | |
| | | | | | | | | | | RAEDT | | | | | | GTCCGCCAGGCTCCAGGGAAGGGGCTG | | PGKGLEWVSG | | | |
| | | | | | | | | | | AVYYC | | | | | | GAGTGGGTCTCAGGAATTAGTGATAGT | | ISDSGGSPYY | | | |
| | | | | | | | | | | | | | | | | GGTGGTAGCCCATACTACGCAGTCTCC | | AVSVRGRFTI | | | |
| | | | | | | | | | | | | | | | | GTGAGGGGCCGATTCACCATCTCCAGA | | SRDNSNNILH | | | |
| | | | | | | | | | | | | | | | | GACAATTCCAACAACATTCTACATCTG | | LQMNSLRAED | | | |
| | | | | | | | | | | | | | | | | CAAATGAACAGCCTGAGAGCCGAGGAC | | TAVYYCAKLA | | | |
| | | | | | | | | | | | | | | | | ACGGCCGTTTATTACTGTGCGAAACTA | | GTPPDYFDYW | | | |
| | | | | | | | | | | | | | | | | GCTGGTACACCCCCCGACTACTTTGAC | | GQGTLVTVSS | | | |
| | | | | | | | | | | | | | | | | TACTGGGGCCAGGGAACCCTGGTCACC | | | | | |
| | | | | | | | | | | | | | | | | GTCTCCTCA | | | | | |
| ADI- | IGHV3- | QVQLVQSG | 10 | FTFRHYG | 220 | WVRQA | 431 | VISYDG | 544 | RFTIS | 801 | AKQSGQ | 1045 | WGQGTL | 1286 | CAGGTGCAGCTGGTGCAGTCTGGGGGA | 1331 | QVQLVQSGGG | 1614 | | |
| 75565 | 30 | GGVVQPGR | | MH | | PGKGL | | DYKYYA | | RDNSR | | YCSGGD | | VTVSS | | GGCGTGGTCCAGCCTGGGAGGTCCCTG | | VVQPGRSLRL | | | |
| | | SLRLSCAA | | | | EWVA | | DSVKG | | STLFL | | CYVGSF | | | | AGACTCTCCTGTGCAGCCTCTGGATTC | | SCAASGFTFR | | | |
| | | SG | | | | | | | | QMNTL | | DY | | | | ACCTTCAGACACTATGGCATGCACTGG | | HYGMHWVRQA | | | |
| | | | | | | | | | | RAEDT | | | | | | GTCCGCCAGGCTCCAGGCAAGGGGCTG | | PGKGLEWVAV | | | |
| | | | | | | | | | | AMYFC | | | | | | GAGTGGGTGGCAGTTATATCATATGAT | | ISYDGDYKYY | | | |
| | | | | | | | | | | | | | | | | GGAGATTATAAATATTATGCAGACTCC | | ADSVKGRFTI | | | |
| | | | | | | | | | | | | | | | | GTGAAGGGCCGATTCACCATCTCCAGA | | SRDNSRSTLF | | | |
| | | | | | | | | | | | | | | | | GACAATTCCAGGAGTACACTGTTTCTG | | LQMNTLRAED | | | |
| | | | | | | | | | | | | | | | | CAAATGAACACCTTGAGAGCTGAAGAC | | TAMYFCAKQS | | | |
| | | | | | | | | | | | | | | | | ACGGCTATGTATTTCTGTGCGAAACAG | | GQYCSGGDCY | | | |
| | | | | | | | | | | | | | | | | TCCGGTCAATATTGTAGTGGTGGAGAC | | VGSFDYWGQG | | | |
| | | | | | | | | | | | | | | | | TGCTACGTAGGGTCCTTTGACTACTGG | | TLVTVSS | | | |
| | | | | | | | | | | | | | | | | GGCCAGGGAACCCTGGTCACCGTCTCC | | | | | |
| | | | | | | | | | | | | | | | | TCA | | | | | |
| ADI- | IGHV3- | QVQLVESG | 31 | FTFDDYG | 222 | WVRQT | 442 | LISGDG | 545 | RFTIS | 802 | TRETTG | 1046 | WGEGTT | 1290 | CAGGTGCAGCTGGTGGAGTCTGGGGGA | 1332 | QVQLVESGGG | 1615 | | |
| 75783 | 43 | GGVVQPGG | | MQ | | PGKDL | | GGTFYA | | RDNSK | | FRYQSL | | VTVSS | | GGCGTGGTACAGCCTGGGGGGTCCCTG | | VVQPGGSLRL | | | |
| | | SLRLSCAA | | | | EWVA | | DSVKG | | NSLYL | | NSYYNY | | | | AGACTCTCCTGTGCAGCCTCTGGATTC | | SCAASGFTFD | | | |
| | | SG | | | | | | | | QMNSL | | MDV | | | | ACCTTTGATGATTATGGCATGCAGTGG | | DYGMQWVRQT | | | |
| | | | | | | | | | | RPEDT | | | | | | GTCCGACAAACTCCAGGGAAGGATCTG | | PGKDLEWVAL | | | |
| | | | | | | | | | | ALYYC | | | | | | GAGTGGGTCGCTCTTATTAGTGGGGAT | | ISGDGGGTFY | | | |
| | | | | | | | | | | | | | | | | GGTGGTGGCACCTTCTATGCAGACTCT | | ADSVKGRFTI | | | |
| | | | | | | | | | | | | | | | | GTGAAGGGCCGATTCACCATCTCCAGA | | SRDNSKNSLY | | | |
| | | | | | | | | | | | | | | | | GACAACAGCAAAAATTCCCTGTATCTG | | LQMNSLRPED | | | |
| | | | | | | | | | | | | | | | | CAAATGAATAGTCTGAGACCTGAGGAC | | TALYYCTRET | | | |
| | | | | | | | | | | | | | | | | ACCGCCTTATATTACTGCACGAGAGAA | | TGFRYQSLNS | | | |
| | | | | | | | | | | | | | | | | ACAACTGGATTCAGATATCAATCCCTT | | YYNYMDVWGE | | | |
| | | | | | | | | | | | | | | | | AATTCCTACTACAACTACATGGACGTC | | GTTVTVSS | | | |
| | | | | | | | | | | | | | | | | TGGGGCGAAGGGACCACGGTCACCGTC | | | | | |
| | | | | | | | | | | | | | | | | TCCTCA | | | | | |
| ADI- | IGHV3- | QVQLVQSG | 32 | FTFRNYA | 223 | WVRQA | 443 | GVSRNG | 546 | RFTIS | 803 | VKDLGQ | 1047 | WGQGTT | 1287 | CAGGTGCAGCTGGTGCAGTCTGGGGGA | 1333 | QVQLVQSGGG | 1616 | | |
| 75805 | 64D | GGLVQPGG | | MH | | PGKGL | | DIADYT | | RDNSK | | QWYDAF | | VTVSS | | GGCTTGGTCCAGCCTGGGGGGTCCCTG | | LVQPGGSLRL | | | |
| | | SLRLSCSA | | | | EYVS | | DSVKG | | NTLYL | | DI | | | | AGACTCTCCTGTTCAGCCTCTGGATTC | | SCSASGFTFR | | | |
| | | SG | | | | | | | | QMRSL | | | | | | ACCTTCAGGAACTATGCTATGCACTGG | | NYAMHWVRQA | | | |
| | | | | | | | | | | GSEDT | | | | | | GTCCGCCAGGCTCCAGGGAAGGGACTG | | PGKGLEYVSG | | | |
| | | | | | | | | | | ALYYC | | | | | | GAGTATGTTTCAGGTGTTAGTCGTAAT | | VSRNGDIADY | | | |
| | | | | | | | | | | | | | | | | GGGGATATCGCAGACTACACAGACTCA | | TDSVKGRFTI | | | |
| | | | | | | | | | | | | | | | | GTGAAGGGCAGATTCACCATCTCCAGA | | SRDNSKNTLY | | | |
| | | | | | | | | | | | | | | | | GACAATTCCAAGAATACGCTGTATCTT | | LQMRSLGSED | | | |
| | | | | | | | | | | | | | | | | CAAATGCGCAGTCTGGGAAGTGAGGAC | | TALYYCVKDL | | | |
| | | | | | | | | | | | | | | | | ACGGCTCTGTATTACTGTGTGAAAGAT | | GQQWYDAFDI | | | |
| | | | | | | | | | | | | | | | | CTTGGCCAGCAGTGGTATGATGCTTTT | | WGQGTTVTVS | | | |
| | | | | | | | | | | | | | | | | GATATCTGGGGCCAAGGGACCACGGTC | | S | | | |
| | | | | | | | | | | | | | | | | ACCGTCTCCTCA | | | | | |
| ADI- | IGHV4- | QVQLQESG | 33 | GSISGIS | 224 | WVRQP | 444 | EIFHSG | 547 | RVTIS | 804 | ARVSGF | 1048 | WGQGTL | 1286 | CAGGTGCAGCTGCAGGAGTCGGGCCCA | 1334 | QVQLQESGPR | 1617 | | |
| 75588 | 4 | PRLVNPSG | | WWS | | PGKGL | | SAHYNP | | VDNSK | | FDY | | VTVSS | | AGACTGGTGAACCCTTCGGGGACCCTG | | LVNPSGTLSL | | | |
| | | TLSLTCAV | | | | EWIG | | SLKS | | NQFSL | | | | | | TCCCTCACCTGCGCTGTCTCTGGGGGC | | TCAVSGGSIS | | | |
| | | SG | | | | | | | | KLTSV | | | | | | TCCATCAGCGGTATTAGCTGGTGGAGT | | GISWWSWVRQ | | | |
| | | | | | | | | | | TAADT | | | | | | TGGGTCCGCCAGCCCCCCGGGAAGGGG | | PPGKGLEWIG | | | |
| | | | | | | | | | | AIYYC | | | | | | CTGGAGTGGATTGGGGAGATATTTCAT | | EIFHSGSAHY | | | |
| | | | | | | | | | | | | | | | | AGTGGGAGCGCCCACTACAACCCGTCC | | NPSLKSRVTI | | | |
| | | | | | | | | | | | | | | | | CTCAAGAGTCGAGTCACCATATCAGTA | | SVDNSKNQFS | | | |
| | | | | | | | | | | | | | | | | GACAACTCCAAGAACCAGTTCTCCCTG | | LKLTSVTAAD | | | |
| | | | | | | | | | | | | | | | | AAGCTGACCTCTGTGACCGCCGCGGAC | | TAIYYCARVS | | | |
| | | | | | | | | | | | | | | | | ACGGCCATATATTACTGTGCAAGAGTC | | GFFDYWGQGT | | | |
| | | | | | | | | | | | | | | | | AGCGGGTTTTTTGACTACTGGGGCCAG | | LVTVSS | | | |
| | | | | | | | | | | | | | | | | GGAACCCTGGTCACCGTCTCCTCA | | | | | |
| ADI- | IGHV4- | QVQLQQWG | 34 | GSLSGYY | 225 | WIRQP | 445 | EINHSG | 548 | RVTIS | 805 | ARGLGL | 1049 | WGQGTT | 1287 | CAGGTGCAGCTACAGCAGTGGGGCGCA | 1335 | QVQLQQWGAG | 1618 | | |
| 75871 | 34 | AGLLKPSE | | CN | | PGKGL | | NTYYNP | | VDTSK | | LDI | | VTVSS | | GGACTGTTGAAGCCTTCGGAGACCCTG | | LLKPSETLSL | | | |
| | | TLSLTCAV | | | | EWIG | | SLKS | | DQFSL | | | | | | TCCCTCACCTGCGCTGTCTCTGGTGGG | | TCAVSGGSLS | | | |
| | | SG | | | | | | | | KLNSV | | | | | | TCCCTCAGTGGTTACTACTGCAACTGG | | GYYCNWIRQP | | | |
| | | | | | | | | | | TAADT | | | | | | ATCCGCCAGCCCCCAGGGAAGGGGCTG | | PGKGLEWIGE | | | |
| | | | | | | | | | | AVYYC | | | | | | GAGTGGATTGGGGAAATCAATCACAGT | | INHSGNTYYN | | | |
| | | | | | | | | | | | | | | | | GGAAACACCTACTACAACCCGTCCCTC | | PSLKSRVTIS | | | |
| | | | | | | | | | | | | | | | | AAGAGTCGAGTCACCATATCAGTAGAC | | VDTSKDQFSL | | | |
| | | | | | | | | | | | | | | | | ACGTCCAAAGACCAGTTCTCCCTGAAG | | KLNSVTAADT | | | |
| | | | | | | | | | | | | | | | | CTGAACTCTGTGACCGCCGCGGACACG | | AVYYCARGLG | | | |
| | | | | | | | | | | | | | | | | GCTGTATATTATTGTGCGAGAGGCTTG | | LLDIWGQGTT | | | |
| | | | | | | | | | | | | | | | | GGACTCCTAGATATCTGGGGCCAAGGG | | VTVSS | | | |
| | | | | | | | | | | | | | | | | ACCACGGTCACCGTCTCCTCA | | | | | |
| ADI- | IGHV1- | QVQLVQSG | 35 | YTFTSYT | 226 | WVRQA | 446 | WIDAGN | 549 | RVTIT | 806 | ARDDSS | 1050 | WGQGTL | 1286 | CAGGTCCAGCTGGTGCAGTCTGGGGCT | 1336 | QVQLVQSGAE | 1619 | | |
| 75868 | 3 | AEVKKPGA | | IH | | PGQRL | | GDTQYS | | SDPSA | | SYSLTS | | VTVSS | | GAGGTGAAGAAGCCTGGGGCCTCAGTG | | VKKPGASVKV | | | |
| | | SVKVSCKA | | | | EWMA | | QNFQG | | STAYM | | YFDY | | | | AAGGTTTCCTGCAAGGCTTCTGGATAC | | SCKASGYTFT | | | |
| | | SG | | | | | | | | ELSGL | | | | | | ACCTTCACTAGCTATACTATTCATTGG | | SYTIHWVRQA | | | |
| | | | | | | | | | | RSEDT | | | | | | GTGCGCCAGGCCCCCGGACAAAGGCTT | | PGQRLEWMAW | | | |
| | | | | | | | | | | AVYYC | | | | | | GAGTGGATGGCATGGATCGACGCTGGC | | IDAGNGDTQY | | | |
| | | | | | | | | | | | | | | | | AATGGTGACACACAATATTCACAGAAC | | SQNFQGRVTI | | | |
| | | | | | | | | | | | | | | | | TTTCAGGGCAGAGTCACCATTACCAGT | | TSDPSASTAY | | | |
| | | | | | | | | | | | | | | | | GACCCATCCGCGAGCACAGCCTACATG | | MELSGLRSED | | | |
| | | | | | | | | | | | | | | | | GAGCTGAGCGGCCTGAGATCTGAAGAC | | TAVYYCARDD | | | |
| | | | | | | | | | | | | | | | | ACGGCTGTCTATTACTGTGCGAGAGAC | | SSSYSLTSYF | | | |
| | | | | | | | | | | | | | | | | GATAGCAGCAGCTATTCCCTAACCTCT | | DYWGQGTLVT | | | |
| | | | | | | | | | | | | | | | | TACTTTGACTACTGGGGCCAGGGAACC | | VSS | | | |
| | | | | | | | | | | | | | | | | CTGGTCACCGTCTCCTCA | | | | | |
| ADI- | IGHV3- | QVQLVQSG | 36 | FIFSDYS | 227 | WVRQA | 447 | GISSTG | 550 | RFTIS | 807 | VKDIGS | 1051 | WGQGTT | 1287 | CAGGTGCAGCTGGTGCAGTCTGGGGGA | 1337 | QVQLVQSGGG | 1620 | | |
| 75653 | 64D | GGLVQPGG | | MH | | PGKGL | | DITDYA | | RDNSK | | WKYFDV | | VTVSS | | GGCTTGGTCCAGCCTGGGGGGTCCCTG | | LVQPGGSLSL | | | |
| | | SLSLSCLV | | | | DYVS | | DSVKD | | NTLYL | | FDV | | | | AGCCTCTCCTGTTTAGTCTCTGGATTC | | SCLVSGFIFS | | | |
| | | SG | | | | | | | | QMSSL | | | | | | ATCTTCAGTGACTATTCTATGCACTGG | | DYSMHWVRQA | | | |
| | | | | | | | | | | RVEDT | | | | | | GTCCGCCAGGCTCCAGGGAAGGGACTG | | PGKGLDYVSG | | | |
| | | | | | | | | | | AVYYC | | | | | | GATTATGTTTCAGGTATTAGTAGTACT | | ISSTGDITDY | | | |
| | | | | | | | | | | | | | | | | GGGGATATCACAGACTACGCAGACTCA | | ADSVKDRFTI | | | |
| | | | | | | | | | | | | | | | | GTGAAGGACAGATTCACCATCTCCAGA | | SRDNSKNTLY | | | |
| | | | | | | | | | | | | | | | | GACAATTCCAAGAACACGCTGTATCTT | | LQMSSLRVED | | | |
| | | | | | | | | | | | | | | | | CAAATGAGCAGTCTGAGAGTTGAGGAC | | TAVYYCVKDI | | | |
| | | | | | | | | | | | | | | | | ACGGCTGTGTATTATTGTGTGAAAGAT | | GSWKYFDVFD | | | |
| | | | | | | | | | | | | | | | | ATTGGGAGCTGGAAGTATTTTGATGTT | | VWGQGTTVTV | | | |
| | | | | | | | | | | | | | | | | TTTGATGTCTGGGGCCAAGGGACCACG | | SS | | | |
| | | | | | | | | | | | | | | | | GTCACCGTCTCCTCA | | | | | |
| ADI- | IGHV3- | QVQLVESG | 11 | FTFSTYS | 228 | WVRQA | 431 | VILYDG | 551 | RFTIS | 792 | ARDWSG | 1052 | WGQGTL | 1286 | CAGGTGCAGCTGGTGGAGTCTGGGGGA | 1338 | QVQLVESGGG | 1621 | | |
| 75826 | 30 | GGVVQPGR | | MH | | PGKGL | | TYKDYA | | RDNSK | | KLPVSW | | VTVSS | | GGCGTGGTCCAGCCTGGGAGGTCCCTG | | VVQPGRSLRL | | | |
| | | SLRLSCAA | | | | EWVA | | DSVKG | | NTLYL | | FDP | | | | AGACTCTCCTGTGCAGCCTCTGGATTC | | SCAASGFTFS | | | |
| | | SG | | | | | | | | QMNSL | | | | | | ACCTTCAGTACCTATTCTATGCACTGG | | TYSMHWVRQA | | | |
| | | | | | | | | | | RPEDT | | | | | | GTCCGCCAGGCTCCAGGCAAGGGGCTG | | PGKGLEWVAV | | | |
| | | | | | | | | | | AVYFC | | | | | | GAGTGGGTGGCAGTTATTTTATATGAT | | ILYDGTYKDY | | | |
| | | | | | | | | | | | | | | | | GGAACCTATAAAGACTACGCAGACTCC | | ADSVKGRFTI | | | |
| | | | | | | | | | | | | | | | | GTGAAGGGCCGATTCACCATCTCCAGA | | SRDNSKNTLY | | | |
| | | | | | | | | | | | | | | | | GACAATTCCAAGAACACGCTGTATCTG | | LQMNSLRPED | | | |
| | | | | | | | | | | | | | | | | CAAATGAACAGCCTGAGACCTGAGGAC | | TAVYFCARDW | | | |
| | | | | | | | | | | | | | | | | ACGGCTGTCTATTTCTGTGCCAGAGAT | | SGKLPVSWFD | | | |
| | | | | | | | | | | | | | | | | TGGAGTGGCAAACTTCCTGTATCCTGG | | PWGQGTLVTV | | | |
| | | | | | | | | | | | | | | | | TTCGACCCCTGGGGCCAGGGAACCCTG | | SS | | | |
| | | | | | | | | | | | | | | | | GTCACCGTCTCCTCA | | | | | |
| ADI- | IGHV3- | QVQLQQSG | 20 | FSFISYG | 229 | WVRQA | 431 | VISDDG | 552 | RFTIS | 808 | ARDDGS | 1053 | WGQGTL | 1286 | CAGGTGCAGCTGCAGCAGTCTGGGGGA | 1339 | QVQLQQSGGG | 1622 | | |
| 75709 | 33 | GGVVQPGR | | MH | | PGKGL | | SFKYSA | | RDNSK | | PTTYFG | | VTVSS | | GGCGTGGTCCAGCCTGGGAGGTCCCTG | | VVQPGRSLRL | | | |
| | | SLRLSCAA | | | | EWVA | | DSVKG | | KTLFL | | L | | | | AGACTCTCCTGTGCAGCGTCTGGATTC | | SCAASGFSFI | | | |
| | | SG | | | | | | | | QMNSL | | | | | | TCCTTCATTAGTTATGGCATGCACTGG | | SYGMHWVRQA | | | |
| | | | | | | | | | | RAEDT | | | | | | GTCCGCCAGGCTCCAGGCAAGGGGCTG | | PGKGLEWVAV | | | |
| | | | | | | | | | | AVYYC | | | | | | GAGTGGGTGGCAGTTATTTCGGATGAT | | ISDDGSFKYS | | | |
| | | | | | | | | | | | | | | | | GGGTCTTTTAAATACTCTGCAGACTCC | | ADSVKGRFTI | | | |
| | | | | | | | | | | | | | | | | GTGAAGGGCCGGTTCACCATCTCCAGA | | SRDNSKKTLF | | | |
| | | | | | | | | | | | | | | | | GACAATTCCAAGAAGACACTGTTTCTG | | LQMNSLRAED | | | |
| | | | | | | | | | | | | | | | | CAAATGAACAGCCTGAGAGCCGAGGAC | | TAVYYCARDD | | | |
| | | | | | | | | | | | | | | | | ACGGCTGTGTATTACTGTGCGAGAGAT | | GSPTTYFGLW | | | |
| | | | | | | | | | | | | | | | | GACGGATCACCCACCACTTACTTTGGC | | GQGTLVTVSS | | | |
| | | | | | | | | | | | | | | | | CTCTGGGGCCAGGGAACCCTGGTCACC | | | | | |
| | | | | | | | | | | | | | | | | GTCTCCTCA | | | | | |
| ADI- | IGHV3- | QVQLVQSG | 10 | FTFSDHA | 230 | WVRQA | 431 | VMSYDG | 553 | RATIS | 809 | AREDYY | 1054 | WGQGTT | 1287 | CAGGTCCAGCTGGTGCAGTCTGGGGGA | 1340 | QVQLVQSGGG | 1623 | | |
| 75685 | 30 | GGVVQPGR | | MH | | PGKGL | | SNEYYA | | RDNSK | | DSSGAF | | VTVSS | | GGCGTGGTCCAGCCTGGGAGGTCCCTG | | VVQPGRSLRL | | | |
| | | SLRLSCAA | | | | EWVA | | DSVKG | | NTLYL | | DM | | | | AGACTCTCCTGTGCAGCCTCTGGATTC | | SCAASGFTFS | | | |
| | | SG | | | | | | | | QMNSL | | | | | | ACCTTCAGTGACCATGCTATGCACTGG | | DHAMHWVRQA | | | |
| | | | | | | | | | | RAEDT | | | | | | GTCCGCCAGGCTCCAGGCAAGGGACTG | | PGKGLEWVAV | | | |
| | | | | | | | | | | AIYYC | | | | | | GAGTGGGTGGCAGTCATGTCATATGAT | | MSYDGSNEYY | | | |
| | | | | | | | | | | | | | | | | GGAAGTAATGAATACTACGCAGATTCC | | ADSVKGRATI | | | |
| | | | | | | | | | | | | | | | | GTGAAGGGCCGAGCCACCATCTCCAGA | | SRDNSKNTLY | | | |
| | | | | | | | | | | | | | | | | GACAATTCCAAGAACACCCTGTATCTG | | LQMNSLRAED | | | |
| | | | | | | | | | | | | | | | | CAAATGAACAGCCTGAGAGCTGAGGAC | | TAIYYCARED | | | |
| | | | | | | | | | | | | | | | | ACGGCTATATATTATTGTGCGAGAGAG | | YYDSSGAFDM | | | |
| | | | | | | | | | | | | | | | | GATTACTATGATAGTAGTGGTGCTTTT | | WGQGTTVTVS | | | |
| | | | | | | | | | | | | | | | | GATATGTGGGGCCAAGGGACCACGGTC | | S | | | |
| | | | | | | | | | | | | | | | | ACCGTCTCCTCA | | | | | |
| ADI- | IGHV3- | QVQLVQSG | 17 | FTFDDYA | 214 | WVRQT | 448 | AISWNS | 554 | RFTIS | 810 | AKDKSS | 1055 | WGQGTT | 1287 | CAGGTCCAGCTGGTGCAGTCTGGGGGA | 1341 | QVQLVQSGGG | 1624 | | |
| 75831 | 9 | GGLVQPGR | | MH | | PGKGL | | ASIGYA | | RDNAK | | NYGDYA | | VTVSS | | GGCTTGGTACAGCCTGGCAGGTCCCTG | | LVQPGRSLRL | | | |
| | | SLRLSCAA | | | | EWVS | | DSVKG | | NSLYL | | GGMEV | | | | AGACTCTCCTGTGCAGCCTCTGGATTC | | SCAASGFTFD | | | |
| | | SG | | | | | | | | QMNSL | | | | | | ACCTTTGATGATTATGCCATGCACTGG | | DYAMHWVRQT | | | |
| | | | | | | | | | | RVEDT | | | | | | GTCCGGCAAACTCCAGGGAAGGGCCTG | | PGKGLEWVSA | | | |
| | | | | | | | | | | ALYYC | | | | | | GAGTGGGTCTCAGCCATTAGTTGGAAT | | ISWNSASIGY | | | |
| | | | | | | | | | | | | | | | | AGTGCTAGTATAGGCTATGCGGACTCT | | ADSVKGRFTI | | | |
| | | | | | | | | | | | | | | | | GTGAAGGGCCGATTCACCATCTCCAGA | | SRDNAKNSLY | | | |
| | | | | | | | | | | | | | | | | GACAACGCCAAGAACTCCCTGTATCTC | | LQMNSLRVED | | | |
| | | | | | | | | | | | | | | | | CAAATGAACAGTCTGAGAGTTGAGGAC | | TALYYCAKDK | | | |
| | | | | | | | | | | | | | | | | ACGGCCTTGTATTACTGTGCAAAAGAT | | SSNYGDYAGG | | | |
| | | | | | | | | | | | | | | | | AAATCGTCAAACTACGGTGACTACGCA | | MEVWGQGTTV | | | |
| | | | | | | | | | | | | | | | | GGGGGTATGGAGGTCTGGGGCCAAGGG | | TVSS | | | |
| | | | | | | | | | | | | | | | | ACCACGGTCACCGTCTCCTCA | | | | | |
| ADI- | IGHV3- | QVQLVQSG | 9 | FTFNSYG | 231 | WVRQA | 431 | VIIHDG | 555 | RFTIS | 811 | AKQSCS | 1056 | WGQGTL | 1286 | CAGGTGCAGCTGGTGCAGTCTGGGGGA | 1342 | QVQLVQSGGA | 1625 | | |
| 75873 | 30 | GAVVQPGR | | MH | | PGKGL | | SNKYHA | | RDNSK | | GGSCYH | | VTVSS | | GCCGTGGTCCAGCCTGGGAGGTCCCTG | | VVQPGRSLRL | | | |
| | | SLRLSCAA | | | | EWVA | | DSVKG | | NTLYL | | EFYFES | | | | AGACTCTCCTGTGCAGCCTCTGGATTC | | SCAASGFTFN | | | |
| | | SG | | | | | | | | QMNNL | | | | | | ACCTTTAATAGCTATGGCATGCACTGG | | SYGMHWVRQA | | | |
| | | | | | | | | | | RAEDT | | | | | | GTCCGCCAGGCTCCGGGCAAGGGGCTG | | PGKGLEWVAV | | | |
| | | | | | | | | | | AVYYC | | | | | | GAGTGGGTGGCAGTTATAATACATGAT | | IIHDGSNKYH | | | |
| | | | | | | | | | | | | | | | | GGAAGTAATAAATACCATGCAGACTCC | | ADSVKGRFTI | | | |
| | | | | | | | | | | | | | | | | GTGAAGGGCCGATTCACCATCTCCAGA | | SRDNSKNTLY | | | |
| | | | | | | | | | | | | | | | | GACAATTCCAAGAACACGTTGTATCTG | | LQMNNLRAED | | | |
| | | | | | | | | | | | | | | | | CAAATGAACAACCTGAGAGCTGAGGAC | | TAVYYCAKQS | | | |
| | | | | | | | | | | | | | | | | ACGGCTGTGTATTACTGTGCGAAACAA | | CSGGSCYHEF | | | |
| | | | | | | | | | | | | | | | | TCATGCAGTGGTGGTAGCTGTTACCAC | | YFESWGQGTL | | | |
| | | | | | | | | | | | | | | | | GAATTTTACTTTGAGTCATGGGGCCAG | | VTVSS | | | |
| | | | | | | | | | | | | | | | | GGAACCCTGGTCACCGTCTCCTCA | | | | | |
| ADI- | IGHV5- | QVQLVQSR | 37 | YSFKYFW | 232 | WVRQM | 449 | TIYPGD | 556 | QVTIS | 812 | VKRQSG | 1057 | WGQGTT | 1287 | CAGGTCCAGCTGGTGCAGTCTCGAGCA | 1343 | QVQLVQSRAE | 1626 | | |
| 75621 | 51 | AEVKKPGD | | IG | | PGKGL | | SDTRYS | | ADRST | | TSFDY | | VTVSS | | GAGGTGAAAAAGCCCGGGGACTCTCTG | | VKKPGDSLQI | | | |
| | | SLQISCQT | | | | EWVA | | LSFQG | | DTAYL | | | | | | CAGATCTCCTGTCAGACATCTGGATAC | | SCQTSGYSFK | | | |
| | | SG | | | | | | | | QWNSL | | | | | | AGTTTTAAGTACTTCTGGATCGGCTGG | | YFWIGWVRQM | | | |
| | | | | | | | | | | KASDT | | | | | | GTGCGCCAGATGCCCGGGAAAGGCCTG | | PGKGLEWVAT | | | |
| | | | | | | | | | | AIYYC | | | | | | GAGTGGGTGGCCACCATCTATCCTGGT | | IYPGDSDTRY | | | |
| | | | | | | | | | | | | | | | | GACTCTGATACCAGATACAGTCTGTCC | | SLSFQGQVTI | | | |
| | | | | | | | | | | | | | | | | TTCCAAGGCCAGGTCACCATCTCAGCC | | SADRSTDTAY | | | |
| | | | | | | | | | | | | | | | | GACAGGTCCACCGATACCGCCTACCTA | | LQWNSLKASD | | | |
| | | | | | | | | | | | | | | | | CAGTGGAACAGCCTCAAGGCCTCGGAC | | TAIYYCVKRQ | | | |
| | | | | | | | | | | | | | | | | ACCGCCATCTACTATTGTGTGAAGCGC | | SGTSFDYWGQ | | | |
| | | | | | | | | | | | | | | | | CAAAGTGGAACTTCGTTTGACTACTGG | | GTTVTVSS | | | |
| | | | | | | | | | | | | | | | | GGCCAGGGGACCACGGTCACCGTCTCC | | | | | |
| | | | | | | | | | | | | | | | | TCA | | | | | |
| ADI- | IGHV3- | QVQLQQSG | 22 | FTFSNYG | 233 | WVRQA | 431 | VIWDDG | 557 | RFTIS | 777 | ARDGPG | 1058 | WGQGTL | 1286 | CAGGTGCAGCTGCAGCAGTCTGGGGGG | 1344 | QVQLQQSGGG | 1627 | | |
| 75755 | 33 | GGVVQPGR | | MH | | PGKGL | | SGKHYA | | RDNSK | | DKMVTL | | VTVSS | | GGCGTGGTCCAGCCTGGGAGGTCCCTG | | VVQPGRSLRL | | | |
| | | SLRLSCEA | | | | EWVA | | DSVKG | | NTLYL | | FDY | | | | AGACTCTCCTGTGAAGCGTCTGGATTC | | SCEASGFTFS | | | |
| | | SG | | | | | | | | QMNSL | | | | | | ACCTTCAGTAATTATGGCATGCACTGG | | NYGMHWVRQA | | | |
| | | | | | | | | | | RAEDT | | | | | | GTCCGCCAGGCTCCAGGCAAGGGGCTG | | PGKGLEWVAV | | | |
| | | | | | | | | | | AVYYC | | | | | | GAGTGGGTGGCAGTTATATGGGATGAT | | IWDDGSGKHY | | | |
| | | | | | | | | | | | | | | | | GGAAGTGGTAAACACTATGCAGACTCC | | ADSVKGRFTI | | | |
| | | | | | | | | | | | | | | | | GTGAAGGGCCGATTCACCATCTCCAGA | | SRDNSKNTLY | | | |
| | | | | | | | | | | | | | | | | GACAACTCCAAGAACACACTTTATCTG | | LQMNSLRAED | | | |
| | | | | | | | | | | | | | | | | CAAATGAACAGCCTGAGAGCCGAGGAC | | TAVYYCARDG | | | |
| | | | | | | | | | | | | | | | | ACGGCTGTGTATTACTGTGCGAGAGAT | | PGDKMVTLFD | | | |
| | | | | | | | | | | | | | | | | GGCCCCGGGGACAAGATGGTTACCCTC | | YWGQGTLVTV | | | |
| | | | | | | | | | | | | | | | | TTTGACTACTGGGGCCAGGGAACCCTG | | SS | | | |
| | | | | | | | | | | | | | | | | GTCACCGTCTCCTCA | | | | | |
| ADI- | IGHV3- | QVQLVQSG | 12 | FSFSNYA | 234 | WVRQA | 437 | GIGGRG | 558 | RFTIS | 813 | ATDEVV | 1059 | WGQGTL | 1286 | CAGGTGCAGCTGGTGCAGTCTGGGGGA | 1345 | QVQLVQSGGG | 1628 | | |
| 75702 | 23 | GGLVQPGG | | MS | | PGKGL | | KNTYYA | | RDNSK | | TAIQPE | | VTVSS | | GGCTTGGTACAGCCGGGGGGGTCCCTG | | LVQPGGSLRL | | | |
| | | SLRLSCAA | | | | EWVS | | DSVKG | | NTLFL | | YFHH | | | | AGACTCTCCTGTGCAGCCTCTGGATTC | | SCAASGFSFS | | | |
| | | SG | | | | | | | | QMNSL | | | | | | AGCTTTAGCAATTACGCCATGAGCTGG | | NYAMSWVRQA | | | |
| | | | | | | | | | | RAEDT | | | | | | GTCCGCCAGGCTCCAGGGAAGGGGCTG | | PGKGLEWVSG | | | |
| | | | | | | | | | | ALYYC | | | | | | GAGTGGGTCTCCGGTATTGGTGGTCGT | | IGGRGKNTYY | | | |
| | | | | | | | | | | | | | | | | GGTAAAAACACATACTACGCAGACTCC | | ADSVKGRFTI | | | |
| | | | | | | | | | | | | | | | | GTGAAGGGCCGGTTCACCATCTCCAGA | | SRDNSKNTLF | | | |
| | | | | | | | | | | | | | | | | GACAATTCCAAGAACACGCTGTTTCTG | | LQMNSLRAED | | | |
| | | | | | | | | | | | | | | | | CAAATGAACAGCCTGAGAGCCGAGGAC | | TALYYCATDE | | | |
| | | | | | | | | | | | | | | | | ACGGCCCTATATTACTGTGCGACAGAT | | VVTAIQPEYF | | | |
| | | | | | | | | | | | | | | | | GAGGTGGTGACTGCTATCCAACCTGAA | | HHWGQGTLVT | | | |
| | | | | | | | | | | | | | | | | TATTTCCACCACTGGGGCCAGGGCACC | | VSS | | | |
| | | | | | | | | | | | | | | | | CTGGTCACCGTCTCCTCA | | | | | |
| ADI- | IGHV3- | QVQLVQSG | 38 | FTFSNYG | 233 | WVRQA | 431 | VIWDDG | 557 | RFTIS | 777 | ARDGPG | 1058 | WGQGTL | 1286 | CAGGTCCAGCTGGTGCAGTCTGGGGGC | 1346 | QVQLVQSGGG | 1629 | | |
| 75778 | 33 | GGVVQPGR | | MH | | PGKGL | | SGKHYA | | RDNSK | | DKMVTL | | VTVSS | | GGCGTGGTCCAGCCTGGGAGGTCCCTG | | VVQPGRSLRL | | | |
| | | SLRLSCEA | | | | EWVA | | DSVKG | | NTLYL | | FDY | | | | AGACTCTCCTGTGAAGCGTCTGGATTC | | SCEASGFTFS | | | |
| | | SG | | | | | | | | QMNSL | | | | | | ACCTTCAGTAATTATGGCATGCACTGG | | NYGMHWVRQA | | | |
| | | | | | | | | | | RAEDT | | | | | | GTCCGCCAGGCTCCAGGCAAGGGGCTG | | PGKGLEWVAV | | | |
| | | | | | | | | | | AVYYC | | | | | | GAGTGGGTGGCAGTTATATGGGATGAT | | IWDDGSGKHY | | | |
| | | | | | | | | | | | | | | | | GGAAGTGGTAAACACTATGCAGACTCC | | ADSVKGRFTI | | | |
| | | | | | | | | | | | | | | | | GTGAAGGGCCGATTCACCATCTCCAGA | | SRDNSKNTLY | | | |
| | | | | | | | | | | | | | | | | GACAACTCCAAGAACACACTTTATCTG | | LQMNSLRAED | | | |
| | | | | | | | | | | | | | | | | CAAATGAACAGCCTGAGAGCCGAGGAC | | TAVYYCARDG | | | |
| | | | | | | | | | | | | | | | | ACGGCTGTGTATTACTGTGCGAGAGAT | | PGDKMVTLFD | | | |
| | | | | | | | | | | | | | | | | GGCCCCGGGGACAAGATGGTTACCCTC | | YWGQGTLVTV | | | |
| | | | | | | | | | | | | | | | | TTTGACTACTGGGGCCAGGGAACCCTG | | SS | | | |
| | | | | | | | | | | | | | | | | GTCACCGTCTCCTCA | | | | | |
| ADI- | IGHV4- | QVQLQESG | 39 | GSISTNG | 235 | WIRQH | 450 | YIYYSG | 559 | RVTMS | 814 | ARDYVT | 1060 | WGQGTL | 1286 | CAGGTGCAGCTGCAGGAGTCGGGCCCA | 1347 | QVQLQESGPG | 1630 | | |
| 75575 | 31 | PGLVKPSQ | | YYWS | | PGKGL | | SAYYNP | | VDTSK | | GRGMLP | | VTVSS | | GGACTGGTGAAGCCCTCACAGACCCTG | | LVKPSQTLSL | | | |
| | | TLSLTCTV | | | | EWIG | | SLKS | | NQFSL | | D | | | | TCCCTCACCTGCACTGTCTCTGGTGGC | | TCTVSGGSIS | | | |
| | | SG | | | | | | | | KLSSV | | | | | | TCCATCAGCACTAATGGTTACTACTGG | | TNGYYWSWIR | | | |
| | | | | | | | | | | TVADT | | | | | | AGCTGGATCCGCCAGCACCCAGGGAAG | | QHPGKGLEWI | | | |
| | | | | | | | | | | AVYYC | | | | | | GGCCTGGAGTGGATTGGGTACATCTAT | | GYIYYSGSAY | | | |
| | | | | | | | | | | | | | | | | TACAGTGGGAGCGCCTACTACAACCCG | | YNPSLKSRVT | | | |
| | | | | | | | | | | | | | | | | TCCCTCAAGAGTCGAGTGACCATGTCA | | MSVDTSKNQF | | | |
| | | | | | | | | | | | | | | | | GTAGACACGTCTAAGAACCAGTTCTCC | | SLKLSSVTVA | | | |
| | | | | | | | | | | | | | | | | CTGAAGCTGAGCTCTGTGACTGTCGCG | | DTAVYYCARD | | | |
| | | | | | | | | | | | | | | | | GACACGGCCGTGTACTACTGTGCGAGA | | YVTGRGMLPD | | | |
| | | | | | | | | | | | | | | | | GACTACGTTACGGGGGGGGGAATGCTC | | WGQGTLVTVS | | | |
| | | | | | | | | | | | | | | | | CCCGACTGGGGCCAGGGAACCCTGGTC | | S | | | |
| | | | | | | | | | | | | | | | | ACCGTCTCCTCA | | | | | |
| ADI- | IGHV7- | QVQLVQSG | 40 | YTFTSNA | 236 | WVRQA | 433 | WINTNT | 560 | RFVFS | 815 | ARRSGY | 1061 | WGQGTL | 1286 | CAGGTGCAGCTGGTGCAGTCTGGGTCT | 1348 | QVQLVQSGSE | 1631 | | |
| 75690 | 4-1 | SESKKPGA | | IN | | PGQGL | | GNPTYA | | LDTAV | | TSGSDW | | VTVSS | | GAGTCGAAAAAGCCTGGGGCCTCAGTG | | SKKPGASVRV | | | |
| | | SVRVSCKA | | | | EWMG | | QGFTG | | STAYL | | LDP | | | | AGGGTTTCCTGCAAGGCTTCTGGATAC | | SCKASGYTFT | | | |
| | | SG | | | | | | | | HINNL | | | | | | ACCTTCACTAGCAATGCTATCAATTGG | | SNAINWVRQA | | | |
| | | | | | | | | | | KPEDT | | | | | | GTGCGACAGGCCCCTGGACAAGGGCTT | | PGQGLEWMGW | | | |
| | | | | | | | | | | AVFYC | | | | | | GAGTGGATGGGATGGATCAACACCAAC | | INTNTGNPTY | | | |
| | | | | | | | | | | | | | | | | ACCGGGAACCCAACGTATGCCCAGGGC | | AQGFTGRFVF | | | |
| | | | | | | | | | | | | | | | | TTCACAGGACGGTTTGTCTTCTCCTTG | | SLDTAVSTAY | | | |
| | | | | | | | | | | | | | | | | GACACCGCTGTCAGCACGGCCTATCTG | | LHINNLKPED | | | |
| | | | | | | | | | | | | | | | | CACATCAACAATCTAAAGCCTGAGGAC | | TAVFYCARRS | | | |
| | | | | | | | | | | | | | | | | ACTGCCGTTTTCTACTGTGCGAGGAGA | | GYTSGSDWLD | | | |
| | | | | | | | | | | | | | | | | TCCGGGTATACAAGTGGCTCAGACTGG | | PWGQGTLVTV | | | |
| | | | | | | | | | | | | | | | | CTCGACCCCTGGGGCCAGGGAACCCTG | | SS | | | |
| | | | | | | | | | | | | | | | | GTCACCGTCTCCTCA | | | | | |
| ADI- | IGHV3- | QVQLVQSG | 41 | FTFGHYG | 237 | WVRQA | 431 | AIIYDG | 561 | RFTIS | 816 | AKLGGL | 1062 | WGQGTL | 1286 | CAGGTGCAGCTGGTGCAGTCTGGGGGA | 1349 | QVQLVQSGGG | 1632 | | |
| 75735 | 30 | GGVGQPGR | | MH | | PGKGL | | SDKQYA | | RDNSK | | YCSGGN | | VTVSS | | GGCGTGGGCCAGCCTGGGAGGTCCCTG | | VGQPGRSLRL | | | |
| | | SLRLSCAA | | | | EWVA | | DSVKG | | NTVYL | | CFSGYF | | | | AGACTCTCCTGTGCAGCCTCTGGATTC | | SCAASGFTFG | | | |
| | | SG | | | | | | | | QMNSL | | DY | | | | ACCTTCGGTCACTATGGCATGCACTGG | | HYGMHWVRQA | | | |
| | | | | | | | | | | RPEDT | | | | | | GTCCGCCAGGCTCCAGGCAAGGGGCTG | | PGKGLEWVAA | | | |
| | | | | | | | | | | AIYFC | | | | | | GAGTGGGTGGCAGCAATAATATATGAT | | IIYDGSDKQY | | | |
| | | | | | | | | | | | | | | | | GGCAGTGACAAACAATATGCAGACTCC | | ADSVKGRFTI | | | |
| | | | | | | | | | | | | | | | | GTGAAGGGCCGATTCACCATCTCCAGA | | SRDNSKNTVY | | | |
| | | | | | | | | | | | | | | | | GACAATTCCAAGAACACAGTGTATTTG | | LQMNSLRPED | | | |
| | | | | | | | | | | | | | | | | CAAATGAACAGCCTGAGACCTGAGGAC | | TAIYFCAKLG | | | |
| | | | | | | | | | | | | | | | | ACGGCTATATATTTTTGTGCGAAACTG | | GLYCSGGNCF | | | |
| | | | | | | | | | | | | | | | | GGGGGCTTGTATTGTAGTGGTGGCAAC | | SGYFDYWGQG | | | |
| | | | | | | | | | | | | | | | | TGCTTCTCCGGCTACTTTGACTACTGG | | TLVTVSS | | | |
| | | | | | | | | | | | | | | | | GGCCAGGGAACCCTGGTCACCGTCTCC | | | | | |
| | | | | | | | | | | | | | | | | TCA | | | | | |
| ADI- | IGHV4- | QVQLQESG | 42 | GSISRSG | 238 | WIRQP | 445 | SIYNSG | 562 | RVTIS | 817 | ARAPGI | 1063 | WGQGTL | 1286 | CAGGTGCAGCTGCAGGAGTCCGGCCCA | 1350 | QVQLQESGPG | 1633 | | |
| 75859 | 39 | PGLVKPSE | | HYWG | | PGKGL | | ATYFNP | | VDTSK | | IDC | | VTVSS | | GGACTGGTGAAGCCTTCGGAGACCCTG | | LVKPSETLSL | | | |
| | | TLSLTCTV | | | | EWIG | | SLKS | | NQFSL | | | | | | TCCCTCACCTGCACTGTGTCTAGTGGC | | TCTVSSGSIS | | | |
| | | SS | | | | | | | | RLSSV | | | | | | TCTATCAGTCGCAGTGGTCACTATTGG | | RSGHYWGWIR | | | |
| | | | | | | | | | | TAADT | | | | | | GGCTGGATCCGCCAGCCCCCAGGGAAG | | QPPGKGLEWI | | | |
| | | | | | | | | | | AVYYC | | | | | | GGGCTGGAGTGGATTGGGAGTATCTAT | | GSIYNSGATY | | | |
| | | | | | | | | | | | | | | | | AATAGTGGGGCCACCTACTTCAACCCG | | FNPSLKSRVT | | | |
| | | | | | | | | | | | | | | | | TCCCTCAAGAGTCGAGTCACCATATCC | | ISVDTSKNQF | | | |
| | | | | | | | | | | | | | | | | GTTGACACGTCCAAGAACCAGTTCTCC | | SLRLSSVTAA | | | |
| | | | | | | | | | | | | | | | | CTGAGGCTGAGCTCTGTGACCGCCGCA | | DTAVYYCARA | | | |
| | | | | | | | | | | | | | | | | GACACGGCTGTCTATTACTGTGCAAGA | | PGIIDCWGQG | | | |
| | | | | | | | | | | | | | | | | GCCCCGGGCATTATTGACTGCTGGGGC | | TLVTVSS | | | |
| | | | | | | | | | | | | | | | | CAGGGAACCCTGGTCACCGTCTCTTCA | | | | | |
| ADI- | IGHV4- | QVQLQQWG | 43 | GSLSGYY | 239 | WIRQP | 445 | EISHSG | 563 | RVTIL | 818 | ARGLGM | 1064 | WGQGTT | 1287 | CAGGTGCAGCTGCAGCAGTGGGGCGCA | 1351 | QVQLQQWGAG | 1634 | | |
| 75857 | 34 | AGLLKPSE | | WS | | PGKGL | | GTYYNP | | VDTSK | | YDV | | VTVSS | | GGACTGTTGAAGCCTTCGGAGACCCTT | | LLKPSETLSL | | | |
| | | TLSLTCAV | | | | EWIG | | SLKS | | NQFSL | | | | | | TCCCTCACCTGCGCTGTCTATGGTGGG | | TCAVYGGSLS | | | |
| | | YG | | | | | | | | KLTSV | | | | | | TCCCTCAGTGGTTACTACTGGAGCTGG | | GYYWSWIRQP | | | |
| | | | | | | | | | | TAADT | | | | | | ATCCGCCAGCCCCCAGGGAAGGGGCTG | | PGKGLEWIGE | | | |
| | | | | | | | | | | AVYYC | | | | | | GAGTGGATTGGGGAAATCAGTCATAGT | | ISHSGGTYYN | | | |
| | | | | | | | | | | | | | | | | GGAGGCACCTACTACAACCCGTCCCTC | | PSLKSRVTIL | | | |
| | | | | | | | | | | | | | | | | AAGAGTCGAGTCACCATATTAGTAGAC | | VDTSKNQFSL | | | |
| | | | | | | | | | | | | | | | | ACGTCCAAGAACCAGTTCTCCCTGAAG | | KLTSVTAADT | | | |
| | | | | | | | | | | | | | | | | CTGACCTCTGTGACCGCCGCGGACACG | | AVYYCARGLG | | | |
| | | | | | | | | | | | | | | | | GCTGTGTATTACTGTGCGAGAGGCCTT | | MYDVWGQGTT | | | |
| | | | | | | | | | | | | | | | | GGAATGTACGACGTCTGGGGCCAAGGG | | VTVSS | | | |
| | | | | | | | | | | | | | | | | ACCACGGTCACCGTCTCCTCA | | | | | |
| ADI- | IGHV4- | QVQLQQSG | 44 | GSISSFN | 240 | WVRQS | 438 | EINHSD | 564 | RVSIS | 819 | ARADRS | 1065 | WGRGTL | 1288 | CAGGTGCAGCTGCAGCAGTCGGGCCCA | 1352 | QVQLQQSGPG | 1635 | | |
| 75596 | 4 | PGLVKPSG | | WWT | | PGKGL | | TTNYNP | | VDKSK | | DYYDST | | VTVSS | | GGACTGGTGAAGCCTTCGGGGACCCTG | | LVKPSGTLSL | | | |
| | | TLSLTCAV | | | | EWIG | | SLKS | | NQFSL | | PYRVPY | | | | TCCCTCACCTGCGCTGTCTCTGGTGGC | | TCAVSGGSIS | | | |
| | | SG | | | | | | | | KLSSV | | FDY | | | | TCCATCAGCAGTTTTAACTGGTGGACT | | SFNWWTWVRQ | | | |
| | | | | | | | | | | TAADT | | | | | | TGGGTCCGCCAGTCCCCAGGAAAGGGG | | SPGKGLEWIG | | | |
| | | | | | | | | | | AVYYC | | | | | | CTGGAATGGATTGGAGAAATCAATCAT | | EINHSDTTNY | | | |
| | | | | | | | | | | | | | | | | AGTGACACCACCAACTACAACCCGTCC | | NPSLKSRVSI | | | |
| | | | | | | | | | | | | | | | | CTCAAGAGTCGAGTCTCCATTTCAGTG | | SVDKSKNQFS | | | |
| | | | | | | | | | | | | | | | | GACAAGTCCAAAAACCAGTTCTCCCTG | | LKLSSVTAAD | | | |
| | | | | | | | | | | | | | | | | AAGTTGAGTTCTGTGACCGCCGCGGAC | | TAVYYCARAD | | | |
| | | | | | | | | | | | | | | | | ACGGCCGTGTATTACTGTGCGAGAGCT | | RSDYYDSTPY | | | |
| | | | | | | | | | | | | | | | | GATCGTAGCGATTACTATGATAGTACT | | RVPYFDYWGR | | | |
| | | | | | | | | | | | | | | | | CCTTATCGAGTCCCCTACTTTGACTAT | | GTLVTVSS | | | |
| | | | | | | | | | | | | | | | | TGGGGCCGGGGAACCCTGGTCACCGTC | | | | | |
| | | | | | | | | | | | | | | | | TCCTCA | | | | | |
| ADI- | IGHV3- | QVQLVQSG | 45 | FSFSSYA | 241 | WVRQA | 431 | VILDDG | 565 | RFTIS | 820 | ARDMGA | 1066 | WGQGTL | 1286 | CAGGTGCAGCTGGTGCAGTCTGGGGGA | 1353 | QVQLVQSGGG | 1636 | | |
| 75838 | 30 | GGVVQPGR | | MH | | PGKGL | | NNKLYA | | RDNSK | | VVTHFD | | VTVSS | | GGCGTGGTCCAGCCTGGGAGGTCCCTG | | VVQPGRSLRL | | | |
| | | SLRLSCVA | | | | EWVA | | DSVKG | | NTLYL | | Y | | | | AGACTCTCCTGTGTAGCCTCTGGATTC | | SCVASGFSFS | | | |
| | | SG | | | | | | | | QVNSL | | | | | | AGCTTCAGTAGTTATGCTATGCACTGG | | SYAMHWVRQA | | | |
| | | | | | | | | | | RTEDT | | | | | | GTCCGCCAGGCTCCAGGCAAGGGGCTA | | PGKGLEWVAV | | | |
| | | | | | | | | | | AVYYC | | | | | | GAATGGGTGGCAGTTATATTAGATGAT | | ILDDGNNKLY | | | |
| | | | | | | | | | | | | | | | | GGAAATAATAAATTGTACGCAGACTCC | | ADSVKGRFTI | | | |
| | | | | | | | | | | | | | | | | GTGAAGGGCCGATTCACCATCTCCAGA | | SRDNSKNTLY | | | |
| | | | | | | | | | | | | | | | | GACAATTCCAAGAACACGCTGTATCTG | | LQVNSLRTED | | | |
| | | | | | | | | | | | | | | | | CAAGTGAACAGCCTGAGAACTGAGGAC | | TAVYYCARDM | | | |
| | | | | | | | | | | | | | | | | ACGGCTGTGTATTACTGTGCGAGAGAT | | GAVVTHFDYW | | | |
| | | | | | | | | | | | | | | | | ATGGGTGCGGTGGTAACTCATTTTGAC | | GQGTLVTVSS | | | |
| | | | | | | | | | | | | | | | | TACTGGGGCCAGGGAACCCTGGTCACC | | | | | |
| | | | | | | | | | | | | | | | | GTCTCCTCA | | | | | |
| ADI- | IGHV3- | QVQLQQSG | 20 | ITFSTFG | 242 | WVRQA | 431 | VISNDG | 566 | RFTIS | 821 | AKEGPG | 1067 | WGQGTL | 1286 | CAGGTGCAGCTGCAGCAGTCTGGGGGA | 1354 | QVQLQQSGGG | 1637 | | |
| 75607 | 30 | GGVVQPGR | | MH | | PGKGL | | GSKHYK | | RDNAK | | ASGFNY | | VTVSS | | GGCGTGGTCCAGCCTGGGAGGTCCCTG | | VVQPGRSLRL | | | |
| | | SLRLSCAA | | | | EWVA | | DSVKG | | NTLYL | | FDS | | | | AGACTCTCCTGTGCAGCCTCTGGAATC | | SCAASGITFS | | | |
| | | SG | | | | | | | | QMNSL | | | | | | ACCTTCAGCACATTTGGAATGCACTGG | | TFGMHWVRQA | | | |
| | | | | | | | | | | RAEDT | | | | | | GTCCGCCAGGCTCCAGGCAAGGGGCTG | | PGKGLEWVAV | | | |
| | | | | | | | | | | AVYYC | | | | | | GAGTGGGTGGCAGTTATATCAAATGAT | | ISNDGGSKHY | | | |
| | | | | | | | | | | | | | | | | GGAGGTAGCAAACACTACAAAGACTCC | | KDSVKGRFTI | | | |
| | | | | | | | | | | | | | | | | GTGAAGGGCCGATTCACCATCTCCAGA | | SRDNAKNTLY | | | |
| | | | | | | | | | | | | | | | | GACAATGCCAAGAACACACTGTATCTG | | LQMNSLRAED | | | |
| | | | | | | | | | | | | | | | | CAAATGAACAGCCTGAGAGCTGAGGAC | | TAVYYCAKEG | | | |
| | | | | | | | | | | | | | | | | ACGGCTGTGTATTACTGTGCGAAAGAG | | PGASGFNYFD | | | |
| | | | | | | | | | | | | | | | | GGGCCCGGGGCTTCGGGATTTAACTAC | | SWGQGTLVTV | | | |
| | | | | | | | | | | | | | | | | TTTGACTCCTGGGGCCAGGGAACCCTG | | SS | | | |
| | | | | | | | | | | | | | | | | GTCACCGTCTCCTCA | | | | | |
| ADI- | IGHV3- | QVQLVQSG | 46 | FTFSSNA | 243 | WVRQA | 437 | GISGRG | 567 | RFTIS | 822 | ATDEVG | 1068 | WGQGTL | 1286 | CAGGTGCAGCTGGTGCAGTCTGGGGGA | 1355 | QVQLVQSGGG | 1638 | | |
| 75570 | 23 | GGLVQPGG | | MS | | PGKGL | | DTTYYA | | RDNSR | | GFTMVQ | | VTVSS | | GGCTTGGTACAGCCTGGGGGGCCCCTG | | LVQPGGPLRL | | | |
| | | PLRLSCAA | | | | EWVS | | DSVKG | | NTLSL | | PEYFHH | | | | AGACTCTCCTGTGCAGCCTCTGGATTC | | SCAASGFTFS | | | |
| | | SG | | | | | | | | QMNSL | | | | | | ACCTTTAGTAGCAATGCCATGAGCTGG | | SNAMSWVRQA | | | |
| | | | | | | | | | | RAEDT | | | | | | GTCCGCCAGGCTCCAGGGAAGGGGCTG | | PGKGLEWVSG | | | |
| | | | | | | | | | | AIYYC | | | | | | GAGTGGGTCTCAGGTATTAGTGGTAGA | | ISGRGDTTYY | | | |
| | | | | | | | | | | | | | | | | GGTGATACCACATACTACGCAGACTCC | | ADSVKGRFTI | | | |
| | | | | | | | | | | | | | | | | GTGAAGGGCCGGTTCACCATCTCCAGA | | SRDNSRNTLS | | | |
| | | | | | | | | | | | | | | | | GACAATTCCAGGAACACGCTGTCTCTG | | LQMNSLRAED | | | |
| | | | | | | | | | | | | | | | | CAGATGAACAGCCTGAGAGCCGAGGAC | | TAIYYCATDE | | | |
| | | | | | | | | | | | | | | | | ACGGCTATATATTACTGTGCGACAGAT | | VGGFTMVQPE | | | |
| | | | | | | | | | | | | | | | | GAAGTAGGGGGATTTACAATGGTTCAG | | YFHHWGQGTL | | | |
| | | | | | | | | | | | | | | | | CCAGAATACTTCCACCACTGGGGCCAG | | VTVSS | | | |
| | | | | | | | | | | | | | | | | GGCACCCTGGTCACCGTCTCCTCA | | | | | |
| ADI- | IGHV3- | QVQLVQSG | 12 | FTFSTYD | 244 | WVRQT | 451 | AIGTAG | 568 | RFTIS | 823 | ARAVES | 1069 | WGQGTT | 1287 | CAGGTCCAGCTGGTGCAGTCTGGGGGA | 1356 | QVQLVQSGGG | 1639 | | |
| 75833 | 13 | GGLVQPGG | | MH | | TGKSL | | DRYYAD | | RENAK | | YNLWAF | | VTVSS | | GGCTTGGTACAGCCTGGGGGGTCCCTG | | LVQPGGSLRL | | | |
| | | SLRLSCAA | | | | EWVS | | SVKG | | NSLYL | | DI | | | | AGACTCTCCTGTGCAGCCTCTGGATTC | | SCAASGFTFS | | | |
| | | SG | | | | | | | | QMNNL | | | | | | ACCTTCAGTACCTACGACATGCACTGG | | TYDMHWVRQT | | | |
| | | | | | | | | | | RAGDT | | | | | | GTCCGCCAAACTACAGGAAAAAGTCTG | | TGKSLEWVSA | | | |
| | | | | | | | | | | AVYHC | | | | | | GAGTGGGTCTCAGCTATTGGTACTGCT | | IGTAGDRYYA | | | |
| | | | | | | | | | | | | | | | | GGTGACAGATACTATGCAGACTCCGTG | | DSVKGRFTIS | | | |
| | | | | | | | | | | | | | | | | AAGGGCCGATTCACCATCTCCAGAGAA | | RENAKNSLYL | | | |
| | | | | | | | | | | | | | | | | AATGCCAAGAACTCCTTGTATCTTCAA | | QMNNLRAGDT | | | |
| | | | | | | | | | | | | | | | | ATGAACAACCTGAGAGCCGGGGACACG | | AVYHCARAVE | | | |
| | | | | | | | | | | | | | | | | GCTGTGTATCACTGTGCAAGAGCGGTC | | SYNLWAFDIW | | | |
| | | | | | | | | | | | | | | | | GAGTCTTATAATTTGTGGGCTTTTGAT | | GQGTTVTVSS | | | |
| | | | | | | | | | | | | | | | | ATCTGGGGCCAAGGGACCACGGTCACC | | | | | |
| | | | | | | | | | | | | | | | | GTCTCCTCA | | | | | |
| ADI- | IGHV4- | QVQLQESG | 21 | APISSSN | 245 | WVRQP | 444 | EIYHTG | 569 | RLTIS | 824 | ARVAGL | 1070 | WGQGTL | 1286 | CAGGTGCAGCTGCAGGAGTCGGGCCCA | 1357 | QVQLQESGPG | 1640 | | |
| 75845 | 4 | PGLVKPSG | | WWS | | PGKGL | | STAYNP | | LDKSN | | FDS | | VTVSS | | GGACTGGTGAAGCCTTCGGGGACCCTG | | LVKPSGTLSL | | | |
| | | TLSLTCAV | | | | EWIG | | SLES | | KQFSL | | | | | | TCCCTCACCTGCGCTGTCTCTGGTGCC | | TCAVSGAPIS | | | |
| | | SG | | | | | | | | MLSSV | | | | | | CCCATCAGCAGTAGTAATTGGTGGAGT | | SSNWWSWVRQ | | | |
| | | | | | | | | | | TAADT | | | | | | TGGGTCCGCCAGCCCCCAGGGAAGGGC | | PPGKGLEWIG | | | |
| | | | | | | | | | | AVYYC | | | | | | CTGGAGTGGATTGGGGAAATCTATCAT | | EIYHTGSTAY | | | |
| | | | | | | | | | | | | | | | | ACTGGGAGCACCGCCTACAACCCGTCC | | NPSLESRLTI | | | |
| | | | | | | | | | | | | | | | | CTCGAGAGTCGACTGACCATATCACTA | | SLDKSNKQFS | | | |
| | | | | | | | | | | | | | | | | GACAAGTCCAACAAGCAATTCTCCCTG | | LMLSSVTAAD | | | |
| | | | | | | | | | | | | | | | | ATGCTGAGCTCTGTGACCGCCGCGGAC | | TAVYYCARVA | | | |
| | | | | | | | | | | | | | | | | ACGGCCGTGTATTACTGTGCGAGAGTA | | GLFDSWGQGT | | | |
| | | | | | | | | | | | | | | | | GCTGGCCTCTTTGACTCCTGGGGTCAG | | LVTVSS | | | |
| | | | | | | | | | | | | | | | | GGAACCCTGGTCACCGTCTCCTCA | | | | | |
| ADI- | IGHV2- | QVQLVESG | 47 | FSLSSPR | 246 | WIRQP | 441 | HIFSND | 570 | RLTIS | 825 | ARNMGL | 1071 | WGQGTL | 1286 | CAGGTGCAGCTGGTGGAGTCTGGTCCT | 1358 | QVQLVESGPV | 1641 | | |
| 75606 | 26 | PVLVKPTE | | MGVS | | PGKAL | | EELFST | | KDTSK | | LEVAGI | | VTVSS | | GTGCTGGTGAAACCCACAGAGACCCTC | | LVKPTETLTL | | | |
| | | TLTLTCAV | | | | EWLA | | SLKS | | SQVVL | | QEGWFD | | | | ACGCTGACCTGCGCCGTCTCTGGGTTC | | TCAVSGFSLS | | | |
| | | SG | | | | | | | | TMTNM | | S | | | | TCACTCAGTAGTCCTAGAATGGGTGTG | | SPRMGVSWIR | | | |
| | | | | | | | | | | GPVDT | | | | | | AGTTGGATCCGTCAGCCCCCAGGGAAG | | QPPGKALEWL | | | |
| | | | | | | | | | | ATYYC | | | | | | GCCCTGGAGTGGCTTGCACACATTTTT | | AHIFSNDEEL | | | |
| | | | | | | | | | | | | | | | | TCGAATGACGAAGAATTGTTCAGTACA | | FSTSLKSRLT | | | |
| | | | | | | | | | | | | | | | | TCTCTGAAGAGCAGGCTCACCATCTCC | | ISKDTSKSQV | | | |
| | | | | | | | | | | | | | | | | AAGGACACCTCCAAAAGCCAGGTGGTC | | VLTMTNMGPV | | | |
| | | | | | | | | | | | | | | | | CTTACCATGACCAACATGGGCCCTGTG | | DTATYYCARN | | | |
| | | | | | | | | | | | | | | | | GACACAGCCACATATTACTGTGCACGG | | MGLLEVAGIQ | | | |
| | | | | | | | | | | | | | | | | AATATGGGTCTTCTGGAAGTGGCTGGA | | EGWFDSWGQG | | | |
| | | | | | | | | | | | | | | | | ATTCAAGAAGGTTGGTTCGACTCCTGG | | TLVTVSS | | | |
| | | | | | | | | | | | | | | | | GGCCAGGGAACCCTGGTCACCGTCTCC | | | | | |
| | | | | | | | | | | | | | | | | TCA | | | | | |
| ADI- | IGHV4- | QVQLQQSG | 44 | GSITNSA | 247 | WVRQS | 452 | EISHSG | 571 | RVTMS | 826 | ARVAGM | 1072 | WGQGTL | 1286 | CAGGTGCAGCTGCAGCAGTCGGGCCCA | 1359 | QVQLQQSGPG | 1642 | | |
| 75568 | 4 | PGLVKPSG | | WWS | | PEKGL | | STNYNP | | VDKSK | | IDY | | VTVSS | | GGACTGGTGAAGCCTTCGGGGACCCTG | | LVKPSGTLSL | | | |
| | | TLSLTCAV | | | | EWIG | | SLKS | | NQFSL | | | | | | TCCCTCACCTGCGCTGTGTCTGGTGGT | | TCAVSGGSIT | | | |
| | | SG | | | | | | | | KLRSV | | | | | | TCCATCACTAATAGTGCCTGGTGGAGT | | NSAWWSWVRQ | | | |
| | | | | | | | | | | TAADT | | | | | | TGGGTCCGCCAGTCCCCAGAAAAGGGG | | SPEKGLEWIG | | | |
| | | | | | | | | | | AVYYC | | | | | | CTGGAGTGGATTGGTGAAATCTCTCAT | | EISHSGSTNY | | | |
| | | | | | | | | | | | | | | | | AGTGGGAGCACCAACTACAACCCGTCC | | NPSLKSRVTM | | | |
| | | | | | | | | | | | | | | | | CTCAAGAGTCGAGTCACCATGTCAGTA | | SVDKSKNQFS | | | |
| | | | | | | | | | | | | | | | | GACAAGTCCAAGAACCAGTTCTCCCTC | | LKLRSVTAAD | | | |
| | | | | | | | | | | | | | | | | AAGTTGAGGTCTGTGACCGCCGCGGAC | | TAVYYCARVA | | | |
| | | | | | | | | | | | | | | | | ACGGCCGTGTATTACTGTGCGCGAGTT | | GMIDYWGQGT | | | |
| | | | | | | | | | | | | | | | | GCGGGGATGATTGACTACTGGGGCCAG | | LVTVSS | | | |
| | | | | | | | | | | | | | | | | GGAACCCTGGTCACCGTCTCCTCA | | | | | |
| ADI- | IGHV3- | PGAAGAVW | 48 | FTFSRFA | 248 | WVRQA | 431 | GISYDG | 572 | RFTIS | 827 | AKEGPG | 1073 | WGQGTL | 1286 | CCAGGTGCAGCTGGTGCAGTCTGGGGG | 1360 | PGAAGAVWGR | 1643 | | |
| 75633 | 30 | GRRGPAGR | | MH | | PGKGL | | ENRYYA | | RDNSK | | ASGFNY | | VTVSS | | AGGCGTGGTCCAGCTGGGAGGTCCCTG | | RGPAGRSLRL | | | |
| | | SLRLSCAA | | | | EWVA | | DSVRG | | NTLYM | | FDY | | | | AGACTCTCCTGTGCAGCCTCTAAATTC | | SCAASKFTFS | | | |
| | | SK | | | | | | | | QMNSL | | | | | | ACCTTCAGTAGGTTTGCCATGCACTGG | | RFAMHWVRQA | | | |
| | | | | | | | | | | RAEDT | | | | | | GTCCGCCAGGCTCCAGGCAAGGGCCTG | | PGKGLEWVAG | | | |
| | | | | | | | | | | AVYYC | | | | | | GAGTGGGTGGCAGGTATCTCATATGAT | | ISYDGENRYY | | | |
| | | | | | | | | | | | | | | | | GGAGAAAATAGATACTATGCAGACTCC | | ADSVRGRFTI | | | |
| | | | | | | | | | | | | | | | | GTGAGGGGCCGATTCACCATCTCCAGA | | SRDNSKNTLY | | | |
| | | | | | | | | | | | | | | | | GACAATTCCAAGAACACGCTGTACATG | | MQMNSLRAED | | | |
| | | | | | | | | | | | | | | | | CAAATGAACAGCCTGAGAGCTGAGGAC | | TAVYYCAKEG | | | |
| | | | | | | | | | | | | | | | | ACGGCTGTCTATTACTGTGCGAAAGAG | | PGASGFNYFD | | | |
| | | | | | | | | | | | | | | | | GGGCCCGGGGCTTCGGGCTTTAACTAC | | YWGQGTLVTV | | | |
| | | | | | | | | | | | | | | | | TTTGACTACTGGGGCCAGGGAACCCTG | | SS | | | |
| | | | | | | | | | | | | | | | | GTCACCGTCTCCTCA | | | | | |
| ADI- | IGHV4- | QVQLQQRG | 49 | DFLRGSY | 249 | WIRQP | 445 | EISQSG | 573 | RLSIS | 828 | ARGRGY | 1074 | WGQGAL | 1285 | CAGGTGCAGCTGCAGCAACGGGGCGCG | 1361 | QVQLQQRGAG | 1644 | | |
| 75617 | 34 | AGLLKPSE | | WA | | PGKGL | | GVNSNP | | IDTSK | | GSGSDS | | VTVSS | | GGACTGTTGAAGCCCTCGGAGACCCTG | | LLKPSETLSL | | | |
| | | TLSLTCAV | | | | EWIG | | SLKS | | SHFSL | | FDD | | | | TCCCTCACCTGCGCTGTCTCTGGTGAT | | TCAVSGDFLR | | | |
| | | SG | | | | | | | | KLKSV | | | | | | TTCTTAAGAGGTTCTTACTGGGCCTGG | | GSYWAWIRQP | | | |
| | | | | | | | | | | TAADT | | | | | | ATCCGCCAGCCCCCCGGGAAGGGGCTG | | PGKGLEWIGE | | | |
| | | | | | | | | | | AIYYC | | | | | | GAGTGGATTGGAGAAATCAGTCAGAGT | | ISQSGGVNSN | | | |
| | | | | | | | | | | | | | | | | GGTGGAGTCAATTCCAACCCGTCCCTC | | PSLKSRLSIS | | | |
| | | | | | | | | | | | | | | | | AAGAGTCGACTCTCCATATCAATAGAC | | IDTSKSHFSL | | | |
| | | | | | | | | | | | | | | | | ACGTCCAAGAGTCACTTCTCCCTGAAG | | KLKSVTAADT | | | |
| | | | | | | | | | | | | | | | | TTGAAGTCTGTGACCGCCGCGGACACG | | AIYYCARGRG | | | |
| | | | | | | | | | | | | | | | | GCTATCTATTACTGTGCGAGAGGCCGC | | YGSGSDSFDD | | | |
| | | | | | | | | | | | | | | | | GGTTACGGATCAGGGAGTGACTCCTTT | | WGQGALVTVS | | | |
| | | | | | | | | | | | | | | | | GACGACTGGGGCCAGGGAGCCCTGGTC | | S | | | |
| | | | | | | | | | | | | | | | | ACCGTCTCCTCA | | | | | |
| ADI- | IGHV3- | QVQLVQSG | 10 | FSFSTYV | 250 | WVRQA | 431 | VISSDG | 574 | RFTIS | 829 | ARDGGF | 1075 | WGQGTT | 1287 | CAGGTGCAGCTGGTGCAGTCTGGGGGA | 1362 | QVQLVQSGGG | 1645 | | |
| 75579 | 30 | GGVVQPGR | | MH | | PGKGL | | TAKFHA | | RDNSK | | HSGRLP | | VTVSS | | GGCGTGGTCCAGCCTGGGAGGTCCCTG | | VVQPGRSLRL | | | |
| | | SLRLSCAA | | | | EWVA | | DSVKG | | NTLYL | | VDAFDI | | | | AGACTCTCCTGTGCAGCCTCTGGATTC | | SCAASGFSFS | | | |
| | | SG | | | | | | | | EMNSL | | | | | | AGCTTCAGTACCTATGTTATGCACTGG | | TYVMHWVRQA | | | |
| | | | | | | | | | | RAEDT | | | | | | GTCCGCCAGGCTCCAGGCAAGGGGCTG | | PGKGLEWVAV | | | |
| | | | | | | | | | | AVFYC | | | | | | GAGTGGGTGGCAGTTATCTCATCTGAT | | ISSDGTAKFH | | | |
| | | | | | | | | | | | | | | | | GGAACCGCTAAATTCCACGCAGACTCC | | ADSVKGRFTI | | | |
| | | | | | | | | | | | | | | | | GTGAAGGGCCGATTCACCATCTCCAGA | | SRDNSKNTLY | | | |
| | | | | | | | | | | | | | | | | GACAATTCCAAGAACACGCTCTATCTA | | LEMNSLRAED | | | |
| | | | | | | | | | | | | | | | | GAAATGAACAGCCTGAGAGCTGAGGAC | | TAVFYCARDG | | | |
| | | | | | | | | | | | | | | | | ACGGCTGTGTTTTACTGTGCGAGAGAT | | GFHSGRLPVD | | | |
| | | | | | | | | | | | | | | | | GGAGGTTTCCACTCTGGGAGGCTCCCT | | AFDIWGQGTT | | | |
| | | | | | | | | | | | | | | | | GTCGATGCTTTTGATATCTGGGGCCAA | | VTVSS | | | |
| | | | | | | | | | | | | | | | | GGGACCACGGTCACCGTCTCCTCA | | | | | |
| ADI- | IGHV1- | QVQLVQSG | 50 | GTFSTYA | 251 | WVRQA | 453 | RIIPVV | 575 | RVTIT | 830 | ARAPLE | 1076 | WGQGTL | 1286 | CAGGTCCAGCTTGTACAGTCTGGGGCT | 1363 | QVQLVQSGAE | 1646 | | |
| 75807 | 69 | AEVKKPGS | | FN | | PGQGL | | NIVNYA | | ADKST | | VGEYGD | | VTVSS | | GAGGTGAAGAAGCCTGGGTCCTCGGTG | | VKKPGSSVKV | | | |
| | | SVKVSCKA | | | | DWMG | | QKFQG | | STAYM | | LMGWFD | | | | AAGGTCTCCTGCAAGGCTTCTGGAGGC | | SCKASGGTFS | | | |
| | | SG | | | | | | | | ELSSL | | P | | | | ACCTTCAGCACCTATGCTTTCAACTGG | | TYAFNWVRQA | | | |
| | | | | | | | | | | RSDDT | | | | | | GTGCGACAGGCCCCTGGACAAGGGCTT | | PGQGLDWMGR | | | |
| | | | | | | | | | | AVYYC | | | | | | GACTGGATGGGAAGGATCATCCCTGTC | | IIPVVNIVNY | | | |
| | | | | | | | | | | | | | | | | GTGAATATAGTAAACTACGCACAGAAG | | AQKFQGRVTI | | | |
| | | | | | | | | | | | | | | | | TTCCAGGGCAGAGTCACGATTACCGCG | | TADKSTSTAY | | | |
| | | | | | | | | | | | | | | | | GACAAATCCACGAGCACTGCCTACATG | | MELSSLRSDD | | | |
| | | | | | | | | | | | | | | | | GAGCTGAGCAGCCTGAGATCTGACGAC | | TAVYYCARAP | | | |
| | | | | | | | | | | | | | | | | ACGGCCGTTTATTACTGTGCGAGAGCG | | LEVGEYGDLM | | | |
| | | | | | | | | | | | | | | | | CCCCTGGAAGTCGGTGAATATGGTGAC | | GWFDPWGQGT | | | |
| | | | | | | | | | | | | | | | | TTAATGGGCTGGTTCGACCCCTGGGGC | | LVTVSS | | | |
| | | | | | | | | | | | | | | | | CAGGGAACCCTGGTCACCGTCTCCTCA | | | | | |
| ADI- | IGHV3- | EVQLVESG | 51 | FTFRNYA | 223 | WVRQA | 443 | GVSRNG | 546 | RFTIS | 831 | VKDLGQ | 1047 | WGQGTT | 1287 | GAGGTGCAGCTGGTGGAGTCTGGGGGA | 1364 | EVQLVESGGG | 1647 | | |
| 75780 | 64D | GGLVQPGG | | MH | | PGKGL | | DIADYT | | RDNSK | | QWYDAF | | VTVSS | | GGCTTGGTCCAGCCTGGGGGGTCCCTG | | LVQPGGSLRL | | | |
| | | SLRLSCSA | | | | EYVS | | DSVKG | | NMVYL | | DI | | | | AGACTCTCCTGTTCAGCCTCTGGATTC | | SCSASGFTFR | | | |
| | | SG | | | | | | | | QMSSL | | | | | | ACCTTCAGAAACTATGCTATGCACTGG | | NYAMHWVRQA | | | |
| | | | | | | | | | | RSEDT | | | | | | GTCCGCCAGGCTCCAGGGAAGGGACTG | | PGKGLEYVSG | | | |
| | | | | | | | | | | ALYYC | | | | | | GAGTATGTTTCAGGTGTTAGTCGTAAT | | VSRNGDIADY | | | |
| | | | | | | | | | | | | | | | | GGGGATATCGCAGACTATACAGACTCA | | TDSVKGRFTI | | | |
| | | | | | | | | | | | | | | | | GTGAAGGGCAGATTCACCATCTCCAGA | | SRDNSKNMVY | | | |
| | | | | | | | | | | | | | | | | GACAATTCCAAGAATATGGTGTATCTT | | LQMSSLRSED | | | |
| | | | | | | | | | | | | | | | | CAAATGAGCAGTCTGAGAAGTGAGGAC | | TALYYCVKDL | | | |
| | | | | | | | | | | | | | | | | ACGGCTCTGTATTACTGTGTGAAAGAT | | GQQWYDAFDI | | | |
| | | | | | | | | | | | | | | | | CTCGGGCAGCAGTGGTATGATGCTTTT | | WGQGTTVTVS | | | |
| | | | | | | | | | | | | | | | | GATATCTGGGGCCAAGGGACAACGGTC | | S | | | |
| | | | | | | | | | | | | | | | | ACCGTCTCCTCA | | | | | |
| ADI- | IGHV1- | QVQLVQSG | 52 | GTFSSYT | 252 | WVRQA | 433 | RIIPIL | 576 | RVTIT | 832 | AREAGY | 1077 | WGRGTL | 1288 | CAGGTCCAGCTGGTGCAGTCTGGGGCT | 1365 | QVQLVQSGAE | 1648 | | |
| 75748 | 69 | AEVKKPGS | | IT | | PGQGL | | GLANYA | | ADKST | | SSSSSS | | VTVSS | | GAGGTGAAGAAGCCTGGGTCCTCGATG | | VKKPGSSMKV | | | |
| | | SMKVSCKA | | | | EWMG | | QKFQG | | STAYM | | WYFDL | | | | AAGGTCTCCTGTAAGGCTTCTGGAGGC | | SCKASGGTFS | | | |
| | | SG | | | | | | | | ELSSL | | | | | | ACCTTCAGCAGCTATACTATCACCTGG | | SYTITWVRQA | | | |
| | | | | | | | | | | RSEDT | | | | | | GTGCGACAGGCCCCTGGACAAGGGCTT | | PGQGLEWMGR | | | |
| | | | | | | | | | | AVYYC | | | | | | GAGTGGATGGGAAGGATCATCCCTATC | | IIPILGLANY | | | |
| | | | | | | | | | | | | | | | | CTTGGTCTAGCAAACTACGCACAGAAG | | AQKFQGRVTI | | | |
| | | | | | | | | | | | | | | | | TTCCAGGGCAGAGTCACGATTACCGCG | | TADKSTSTAY | | | |
| | | | | | | | | | | | | | | | | GACAAATCCACGAGCACAGCCTACATG | | MELSSLRSED | | | |
| | | | | | | | | | | | | | | | | GAGCTGAGCAGCCTGAGATCTGAGGAC | | TAVYYCAREA | | | |
| | | | | | | | | | | | | | | | | ACGGCCGTGTATTACTGTGCGAGAGAG | | GYSSSSSSWY | | | |
| | | | | | | | | | | | | | | | | GCGGGGTATAGTAGCTCGTCGAGTAGC | | FDLWGRGTLV | | | |
| | | | | | | | | | | | | | | | | TGGTACTTCGATCTCTGGGGCCGTGGC | | TVSS | | | |
| | | | | | | | | | | | | | | | | ACCCTGGTCACCGTCTCCTCA | | | | | |
| ADI- | IGHV1- | QVQLVQSG | 53 | YTFTKYY | 253 | WVRQA | 433 | GINPSG | 577 | RVTMT | 833 | ARADFS | 1078 | WGQGTL | 1286 | CAGGTCCAGCTGGTGCAGTCTGGGGCT | 1366 | QVQLVQSGAE | 1649 | | |
| 75820 | 46 | AEVKKPGA | | IH | | PGQGL | | GSTDYA | | SDTST | | DSVVWA | | VTVSS | | GAGGTGAAGAAGCCTGGGGCCTCAGTG | | VKKPGASVKV | | | |
| | | SVKVPCKA | | | | EWMG | | QKFQG | | STVYM | | VSYFFD | | | | AAGGTTCCCTGCAAGGCATCTGGATAC | | PCKASGYTFT | | | |
| | | SG | | | | | | | | ELSSL | | S | | | | ACCTTCACCAAATACTATATACACTGG | | KYYIHWVRQA | | | |
| | | | | | | | | | | RSEDT | | | | | | GTGCGACAGGCCCCTGGACAAGGGCTT | | PGQGLEWMGG | | | |
| | | | | | | | | | | AVYFC | | | | | | GAGTGGATGGGAGGAATCAACCCCAGT | | INPSGGSTDY | | | |
| | | | | | | | | | | | | | | | | GGTGGTAGCACAGACTACGCACAGAAG | | AQKFQGRVTM | | | |
| | | | | | | | | | | | | | | | | TTCCAGGGCAGAGTCACCATGACCAGC | | TSDTSTSTVY | | | |
| | | | | | | | | | | | | | | | | GACACGTCCACGAGCACAGTCTACATG | | MELSSLRSED | | | |
| | | | | | | | | | | | | | | | | GAACTGAGCAGCCTGAGATCTGAGGAC | | TAVYFCARAD | | | |
| | | | | | | | | | | | | | | | | ACGGCCGTATATTTCTGTGCGAGAGCC | | FSDSVVWAVS | | | |
| | | | | | | | | | | | | | | | | GACTTTAGCGATTCCGTCGTCTGGGCA | | YFFDSWGQGT | | | |
| | | | | | | | | | | | | | | | | GTCTCCTACTTCTTTGACTCCTGGGGC | | LVTVSS | | | |
| | | | | | | | | | | | | | | | | CAGGGAACCCTGGTCACCGTCTCCTCA | | | | | |
| ADI- | IGHV1- | QVQLVQSG | 54 | YTFTGYY | 254 | WVRQA | 433 | RINPNS | 578 | RVTMT | 834 | ARALGC | 1079 | WGQGTL | 1286 | CAGGTGCAGCTGGTGCAGTCTGGGGCT | 1367 | QVQLVQSGAE | 1650 | | |
| 75612 | 2 | AEVKKPGA | | IH | | PGQGL | | GGTNYA | | RDTSV | | SDDNCY | | VTVSS | | GAGGTGAAGAAGCCTGGGGCCTCAGTG | | VKKPGASVNV | | | |
| | | SVNVSCKA | | | | EWMG | | QKFQG | | STAYM | | GRRDY | | | | AACGTCTCCTGCAAGGCTTCTGGATAC | | SCKASGYTFT | | | |
| | | SG | | | | | | | | ELRRL | | | | | | ACCTTCACCGGCTACTATATCCACTGG | | GYYIHWVRQA | | | |
| | | | | | | | | | | TSDDT | | | | | | GTGCGACAGGCCCCTGGACAAGGGCTT | | PGQGLEWMGR | | | |
| | | | | | | | | | | AVYYC | | | | | | GAGTGGATGGGACGGATCAACCCTAAC | | INPNSGGTNY | | | |
| | | | | | | | | | | | | | | | | AGTGGTGGCACAAACTATGCACAGAAG | | AQKFQGRVTM | | | |
| | | | | | | | | | | | | | | | | TTTCAGGGCAGGGTCACCATGACCAGG | | TRDTSVSTAY | | | |
| | | | | | | | | | | | | | | | | GACACGTCCGTCAGCACAGCCTACATG | | MELRRLTSDD | | | |
| | | | | | | | | | | | | | | | | GAGCTGCGCAGGCTGACATCTGACGAC | | TAVYYCARAL | | | |
| | | | | | | | | | | | | | | | | ACGGCCGTATATTACTGTGCGAGAGCC | | GCSDDNCYGR | | | |
| | | | | | | | | | | | | | | | | CTAGGTTGTAGTGATGATAACTGCTAC | | RDYWGQGTLV | | | |
| | | | | | | | | | | | | | | | | GGACGAAGGGACTACTGGGGCCAGGGA | | TVSS | | | |
| | | | | | | | | | | | | | | | | ACCCTGGTCACCGTCTCCTCA | | | | | |
| ADI- | IGHV3- | QVQLVQSG | 55 | FSFSNYA | 234 | WVRQA | 437 | GISGRG | 579 | RFTVS | 835 | AKDQEG | 1080 | WGQGTL | 1286 | CAGGTGCAGCTGGTGCAGTCTGGGGGA | 1368 | QVQLVQSGGD | 1651 | EVQLVESG | 1873 |
| 75696 | 23 | GDLVQPGG | | MS | | PGKGL | | DRTYYA | | RDNSK | | GFTMVQ | | VTVSS | | GACTTGGTACAGCCTGGGGGGTCCCTG | | LVQPGGSLRL | | GDLVQPGG | |
| | | SLRLSCTA | | | | EWVS | | DSVKG | | NRLYL | | PEYFHY | | | | AGACTCTCCTGTACAGCCTCTGGATTC | | SCTASGFSFS | | SLRLSCTA | |
| | | SG | | | | | | | | QMNSL | | | | | | AGTTTTAGCAACTATGCCATGAGCTGG | | NYAMSWVRQA | | SGFSFSNY | |
| | | | | | | | | | | TDEDT | | | | | | GTCCGCCAGGCTCCAGGGAAGGGGCTG | | PGKGLEWVSG | | AMSWVRQA | |
| | | | | | | | | | | AIYYC | | | | | | GAGTGGGTCTCAGGTATTAGTGGTCGT | | ISGRGDRTYY | | PGKGLEWV | |
| | | | | | | | | | | | | | | | | GGTGATAGGACATACTACGCAGACTCC | | ADSVKGRFTV | | SGISGRGD | |
| | | | | | | | | | | | | | | | | GTGAAGGGCCGGTTCACCGTCTCCAGA | | SRDNSKNRLY | | RTYYADSV | |
| | | | | | | | | | | | | | | | | GACAATTCCAAGAACAGGCTGTATCTG | | LQMNSLTDED | | KGRFTVSR | |
| | | | | | | | | | | | | | | | | CAAATGAATAGCCTGACAGACGAAGAC | | TAIYYCAKDQ | | DNSKNRLY | |
| | | | | | | | | | | | | | | | | ACGGCCATATATTACTGTGCAAAAGAT | | EGGFTMVQPE | | LQMNSLTD | |
| | | | | | | | | | | | | | | | | CAAGAAGGGGGGTTTACTATGGTTCAG | | YFHYWGQGTL | | EDTAIYYC | |
| | | | | | | | | | | | | | | | | CCAGAATACTTCCACTACTGGGGCCAG | | VTVSS | | AKDQEGGF | |
| | | | | | | | | | | | | | | | | GGCACCCTGGTCACCGTCTCCTCA | | | | TMVQPEYF | |
| | | | | | | | | | | | | | | | | | | | | HYWGQGTL | |
| | | | | | | | | | | | | | | | | | | | | VTVSS | |
| ADI- | IGHV4- | QVQLQQSG | 56 | GSVSSGS | 255 | WIRQH | 450 | NIFYNG | 580 | RVIIS | 836 | ARSPGG | 1081 | WGQGTL | 1286 | CAGGTGCAGCTGCAGCAGTCGGGCCCA | 1369 | QVQLQQSGPG | 1652 | | |
| 75571 | 31 | PGLVKPSQ | | NYWS | | PGKGL | | KTYYNP | | LNTSK | | LFDF | | VTVSS | | GGACTGGTGAAGCCTTCACAGACCCTG | | LVKPSQTLSL | | | |
| | | TLSLTCTV | | | | EWIG | | SLKS | | NHFSL | | | | | | TCCCTCACCTGCACTGTCTCTGGTGGC | | TCTVSGGSVS | | | |
| | | SG | | | | | | | | ELNSV | | | | | | TCCGTCAGCAGTGGTAGTAACTACTGG | | SGSNYWSWIR | | | |
| | | | | | | | | | | TAADT | | | | | | AGCTGGATCCGCCAGCACCCAGGGAAG | | QHPGKGLEWI | | | |
| | | | | | | | | | | AVYYC | | | | | | GGCCTGGAGTGGATTGGGAACATCTTT | | GNIFYNGKTY | | | |
| | | | | | | | | | | | | | | | | TACAATGGGAAAACCTACTACAACCCG | | YNPSLKSRVI | | | |
| | | | | | | | | | | | | | | | | TCCCTAAAGAGTCGAGTTATCATATCA | | ISLNTSKNHF | | | |
| | | | | | | | | | | | | | | | | CTAAATACGTCTAAGAACCACTTCTCC | | SLELNSVTAA | | | |
| | | | | | | | | | | | | | | | | CTGGAGCTGAACTCTGTGACTGCCGCG | | DTAVYYCARS | | | |
| | | | | | | | | | | | | | | | | GACACGGCCGTTTATTACTGTGCGAGG | | PGGLFDFWGQ | | | |
| | | | | | | | | | | | | | | | | TCCCCTGGGGGATTATTTGACTTCTGG | | GTLVTVSS | | | |
| | | | | | | | | | | | | | | | | GGCCAGGGAACCCTGGTCACCGTCTCC | | | | | |
| | | | | | | | | | | | | | | | | TCA | | | | | |
| ADI- | IGHV3- | QVQLVQSG | 57 | FAFDAYD | 256 | WVRQV | 454 | LISRDG | 581 | RFTIS | 837 | VKDISA | 1082 | WGQGTL | 1286 | CAGGTGCAGCTGGTACAGTCTGGGGGA | 1370 | QVQLVQSGGG | 1653 | | |
| 75766 | 43 | GGVVQPGG | | MH | | PEKGL | | GGTFYA | | RDNSK | | DTSMVP | | VTVSS | | GGCGTGGTACAGCCTGGGGGGTCCCTG | | VVQPGGSLRL | | | |
| | | SLRLSCAA | | | | EWIS | | DSMKG | | NSLYL | | VFAH | | | | AGACTCTCCTGTGCAGCCTCTGGATTC | | SCAASGFAFD | | | |
| | | SG | | | | | | | | QMNSL | | | | | | GCCTTTGATGCTTATGACATGCACTGG | | AYDMHWVRQV | | | |
| | | | | | | | | | | RTEDT | | | | | | GTCCGTCAAGTTCCAGAGAAGGGTCTG | | PEKGLEWISL | | | |
| | | | | | | | | | | ALYYC | | | | | | GAGTGGATCTCTCTTATTAGTAGGGAT | | ISRDGGGTFY | | | |
| | | | | | | | | | | | | | | | | GGTGGTGGCACATTCTATGCAGACTCT | | ADSMKGRFTI | | | |
| | | | | | | | | | | | | | | | | ATGAAGGGCCGATTCACCATCTCCAGA | | SRDNSKNSLY | | | |
| | | | | | | | | | | | | | | | | GACAACAGCAAAAACTCCCTGTATTTG | | LQMNSLRTED | | | |
| | | | | | | | | | | | | | | | | CAAATGAACAGTCTGAGAACTGAGGAC | | TALYYCVKDI | | | |
| | | | | | | | | | | | | | | | | ACCGCCTTGTATTACTGTGTAAAAGAT | | SADTSMVPVF | | | |
| | | | | | | | | | | | | | | | | ATTTCCGCGGATACTTCTATGGTACCG | | AHWGQGTLVT | | | |
| | | | | | | | | | | | | | | | | GTCTTTGCTCACTGGGGCCAGGGAACC | | VSS | | | |
| | | | | | | | | | | | | | | | | CTGGTCACCGTCTCCTCA | | | | | |
| ADI- | IGHV4- | QVQLQQSG | 44 | GSISGIN | 257 | WVRQS | 455 | EISQSG | 582 | RVTIS | 838 | ARVSGF | 1083 | WGQGTL | 1286 | CAGGTGCAGCTGCAGCAGTCGGGCCCA | 1371 | QVQLQQSGPG | 1654 | | |
| 75635 | 4 | PGLVKPSG | | WWS | | PRQGL | | STYYNP | | VDKSK | | FDS | | VTVSS | | GGACTGGTGAAGCCTTCGGGGACCCTG | | LVKPSGTLSL | | | |
| | | TLSLTCAV | | | | EWIG | | SLKS | | NQFSL | | | | | | TCCCTCACCTGCGCTGTCTCTGGTGGC | | TCAVSGGSIS | | | |
| | | SG | | | | | | | | KLTSV | | | | | | TCCATCAGTGGTATAAACTGGTGGAGT | | GINWWSWVRQ | | | |
| | | | | | | | | | | TAADT | | | | | | TGGGTCCGCCAGTCCCCACGGCAGGGG | | SPRQGLEWIG | | | |
| | | | | | | | | | | AIYYC | | | | | | CTGGAGTGGATTGGGGAAATCTCTCAG | | EISQSGSTYY | | | |
| | | | | | | | | | | | | | | | | AGTGGGAGCACCTACTACAACCCGTCT | | NPSLKSRVTI | | | |
| | | | | | | | | | | | | | | | | CTCAAAAGTCGAGTCACCATATCAGTA | | SVDKSKNQFS | | | |
| | | | | | | | | | | | | | | | | GACAAGTCTAAGAACCAGTTCTCCCTG | | LKLTSVTAAD | | | |
| | | | | | | | | | | | | | | | | AAGCTGACCTCTGTGACCGCCGCGGAC | | TAIYYCARVS | | | |
| | | | | | | | | | | | | | | | | ACGGCCATATATTACTGTGCGAGAGTC | | GFFDSWGQGT | | | |
| | | | | | | | | | | | | | | | | AGCGGGTTTTTTGACTCCTGGGGCCAG | | LVTVSS | | | |
| | | | | | | | | | | | | | | | | GGAACCCTGGTCACCGTCTCCTCA | | | | | |
| ADI- | IGHV2- | RCPLKESG | 58 | FSLNTGG | 258 | WIRQP | 441 | LIYWDG | 583 | RLTIT | 839 | ARHGIP | 1084 | WGQGTL | 1286 | AGGTGCCCGTTGAAGGAGTCTGGTCCT | 1372 | RCPLKESGPT | 1655 | QITLKESG | 1874 |
| 75864 | 5 | PTVVKPTQ | | VGVG | | PGKAL | | DQRYSP | | KDTSK | | TIFDY | | VTVSS | | ACGGTGGTTAAACCCACACAGACCCTC | | VVKPTQTLTL | | PTVVKPTQ | |
| | | TLTLTCTF | | | | EWLA | | SLNT | | NQVVL | | | | | | ACGCTGACCTGCACCTTCTCTGGGTTC | | TCTFSGFSLN | | TLTLTCTF | |
| | | SG | | | | | | | | TMTNM | | | | | | TCACTCAACACTGGTGGAGTGGGTGTG | | TGGVGVGWIR | | SGFSLNTG | |
| | | | | | | | | | | DPVDT | | | | | | GGCTGGATCCGTCAGCCCCCAGGAAAG | | QPPGKALEWL | | GVGVGWIR | |
| | | | | | | | | | | ATYFC | | | | | | GCCCTGGAGTGGCTTGCGCTCATTTAT | | ALIYWDGDQR | | QPPGKALE | |
| | | | | | | | | | | | | | | | | TGGGATGGTGATCAGCGCTACAGCCCA | | YSPSLNTRLT | | WLALIYWD | |
| | | | | | | | | | | | | | | | | TCTCTGAATACCAGACTCACCATCACC | | ITKDTSKNQV | | GDQRYSPS | |
| | | | | | | | | | | | | | | | | AAGGACACCTCCAAAAACCAGGTAGTC | | VLTMTNMDPV | | LNTRLTIT | |
| | | | | | | | | | | | | | | | | CTTACAATGACCAACATGGACCCTGTG | | DTATYFCARH | | KDTSKNQV | |
| | | | | | | | | | | | | | | | | GACACAGCCACATATTTCTGTGCACGC | | GIPTIFDYWG | | VLTMTNMD | |
| | | | | | | | | | | | | | | | | CATGGCATTCCTACAATTTTTGATTAC | | QGTLVTVSS | | PVDTATYF | |
| | | | | | | | | | | | | | | | | TGGGGCCAGGGAACCCTGGTCACCGTC | | | | CARHGIPT | |
| | | | | | | | | | | | | | | | | TCCTCA | | | | IFDYWGQG | |
| | | | | | | | | | | | | | | | | | | | | TLVTVSS | |
| ADI- | IGHV1- | QVQLVQSG | 59 | YSFTSYS | 259 | WVRQA | 433 | IINPGG | 584 | RVTVT | 840 | ARTRIS | 1085 | WGQGTL | 1286 | CAGGTGCAGCTGGTGCAGTCTGGGGCT | 1373 | QVQLVQSGAE | 1656 | | |
| 75877 | 46 | AEVRKPGA | | MH | | PGQGL | | GSTGYA | | RDTST | | PATDYF | | VTVSS | | GAGGTGAGGAAGCCTGGGGCCTCAGTG | | VRKPGASVKV | | | |
| | | SVKVSCEA | | | | EWMG | | QKFQG | | STVYM | | DK | | | | AAAGTTTCCTGCGAGGCATCTGGATAC | | SCEASGYSFT | | | |
| | | SG | | | | | | | | ELSSL | | | | | | AGCTTCACCAGCTATTCTATGCACTGG | | SYSMHWVRQA | | | |
| | | | | | | | | | | RSEDT | | | | | | GTGCGCCAGGCCCCTGGACAAGGACTT | | PGQGLEWMGI | | | |
| | | | | | | | | | | AMYYC | | | | | | GAGTGGATGGGTATAATCAACCCTGGT | | INPGGGSTGY | | | |
| | | | | | | | | | | | | | | | | GGTGGTAGCACAGGCTACGCACAGAAG | | AQKFQGRVTV | | | |
| | | | | | | | | | | | | | | | | TTCCAGGGCAGAGTCACCGTGACCAGG | | TRDTSTSTVY | | | |
| | | | | | | | | | | | | | | | | GACACGTCCACGAGCACAGTCTACATG | | MELSSLRSED | | | |
| | | | | | | | | | | | | | | | | GAACTGAGCAGCCTGAGATCTGAGGAC | | TAMYYCARTR | | | |
| | | | | | | | | | | | | | | | | ACGGCCATGTATTACTGTGCGAGAACC | | ISPATDYFDK | | | |
| | | | | | | | | | | | | | | | | CGCATATCACCAGCCACGGACTACTTT | | WGQGTLVTVS | | | |
| | | | | | | | | | | | | | | | | GACAAGTGGGGCCAGGGAACCCTGGTC | | S | | | |
| | | | | | | | | | | | | | | | | ACCGTCTCCTCA | | | | | |
| ADI- | IGHV3- | QVQLVESG | 30 | FIVSSNY | 260 | WVRQA | 437 | TIFSGG | 585 | RFIIS | 841 | VRDGLY | 1086 | WGQGTL | 1286 | CAGGTCCAGCTGGTGGAGTCTGGAGGA | 1374 | QVQLVESGGG | 1657 | | |
| 75827 | 53 | GGLIQPGG | | MS | | PGKGL | | STFYAD | | RDNSK | | VGVAD | | VTVSS | | GGCTTGATCCAGCCTGGGGGGTCCCTG | | LIQPGGSLRL | | | |
| | | SLRLSCAA | | | | EWVS | | SVKG | | NTLYL | | | | | | AGACTCTCCTGTGCAGCCTCTGGATTC | | SCAASGFIVS | | | |
| | | SG | | | | | | | | QMTSL | | | | | | ATCGTCAGTAGCAACTACATGAGCTGG | | SNYMSWVRQA | | | |
| | | | | | | | | | | RAGDT | | | | | | GTCCGCCAGGCTCCAGGGAAGGGGCTG | | PGKGLEWVST | | | |
| | | | | | | | | | | AVYYC | | | | | | GAGTGGGTCTCAACCATTTTTAGCGGT | | IFSGGSTFYA | | | |
| | | | | | | | | | | | | | | | | GGAAGCACGTTCTACGCAGACTCCGTG | | DSVKGRFIIS | | | |
| | | | | | | | | | | | | | | | | AAGGGCCGATTCATCATCTCCAGAGAC | | RDNSKNTLYL | | | |
| | | | | | | | | | | | | | | | | AATTCCAAGAACACGCTGTATCTTCAA | | QMTSLRAGDT | | | |
| | | | | | | | | | | | | | | | | ATGACCAGCCTGAGAGCCGGGGACACG | | AVYYCVRDGL | | | |
| | | | | | | | | | | | | | | | | GCCGTATATTATTGTGTGAGAGATGGA | | YVGVADWGQG | | | |
| | | | | | | | | | | | | | | | | CTATATGTGGGGGTGGCGGACTGGGGC | | TLVTVSS | | | |
| | | | | | | | | | | | | | | | | CAGGGAACCCTGGTCACCGTCTCCTCA | | | | | |
| ADI- | IGHV3- | QVQLVQSG | 60 | FIFSDYS | 227 | WVRQA | 447 | GISSTG | 550 | RFTIS | 842 | VKDIGS | 1051 | WGQGTT | 1287 | CAGGTGCAGCTGGTGCAGTCTGGGGGA | 1375 | QVQLVQSGGA | 1658 | | |
| 75683 | 64D | GALVQPGG | | MH | | PGKGL | | DITDYA | | RDNSK | | WKYFDV | | VTVSS | | GCCTTGGTCCAGCCTGGGGGGTCCCTG | | LVQPGGSLSL | | | |
| | | SLSLSCLV | | | | DYVS | | DSVKD | | NMLYL | | FDV | | | | AGCCTCTCCTGTTTAGTCTCTGGATTC | | SCLVSGFIFS | | | |
| | | SG | | | | | | | | QMSSL | | | | | | ATCTTCAGTGACTATTCTATGCACTGG | | DYSMHWVRQA | | | |
| | | | | | | | | | | RVEDT | | | | | | GTCCGCCAGGCTCCAGGGAAGGGACTG | | PGKGLDYVSG | | | |
| | | | | | | | | | | AVYYC | | | | | | GATTATGTTTCAGGTATTAGTAGTACA | | ISSTGDITDY | | | |
| | | | | | | | | | | | | | | | | GGGGATATCACAGACTACGCAGACTCA | | ADSVKDRFTI | | | |
| | | | | | | | | | | | | | | | | GTGAAGGACAGATTCACCATCTCCAGA | | SRDNSKNMLY | | | |
| | | | | | | | | | | | | | | | | GACAATTCCAAGAACATGCTGTATCTT | | LQMSSLRVED | | | |
| | | | | | | | | | | | | | | | | CAAATGAGCAGTTTGAGAGTTGAGGAC | | TAVYYCVKDI | | | |
| | | | | | | | | | | | | | | | | ACGGCTGTGTATTATTGTGTGAAAGAT | | GSWKYFDVFD | | | |
| | | | | | | | | | | | | | | | | ATTGGGAGCTGGAAGTACTTTGATGTT | | VWGQGTTVTV | | | |
| | | | | | | | | | | | | | | | | TTTGATGTCTGGGGCCAAGGGACCACG | | SS | | | |
| | | | | | | | | | | | | | | | | GTCACCGTCTCCTCA | | | | | |
| ADI- | IGHV4- | QVQLQESG | 61 | GSISTSY | 261 | WVRQP | 444 | EIYQSG | 586 | RVTIS | 843 | VRDRSL | 1087 | WGKGTT | 1291 | CAGGTGCAGCTGCAGGAGTCGGGCCCA | 1376 | QVQLQESGPG | 1659 | | |
| 75776 | 4 | PGLVKPSG | | WWS | | PGKGL | | STNYNP | | VDRSK | | YCSNGN | | VTVSS | | GGACTGGTGAAGCCTTCGGGGACCCTG | | LVKPSGTLSL | | | |
| | | TLSLTCDV | | | | EWIG | | SLKS | | NQFSL | | CPHMDV | | | | TCCCTCACCTGCGATGTCTCTGGTGGC | | TCDVSGGSIS | | | |
| | | SG | | | | | | | | KLSSV | | | | | | TCCATCAGCACTAGTTACTGGTGGAGC | | TSYWWSWVRQ | | | |
| | | | | | | | | | | TAADT | | | | | | TGGGTCCGCCAGCCCCCAGGAAAGGGG | | PPGKGLEWIG | | | |
| | | | | | | | | | | GVYYC | | | | | | CTGGAGTGGATTGGGGAAATCTATCAA | | EIYQSGSTNY | | | |
| | | | | | | | | | | | | | | | | AGTGGGAGCACCAACTACAACCCGTCC | | NPSLKSRVTI | | | |
| | | | | | | | | | | | | | | | | CTCAAGAGTCGAGTCACCATATCAGTA | | SVDRSKNQFS | | | |
| | | | | | | | | | | | | | | | | GACAGGTCCAAGAACCAGTTCTCCCTG | | LKLSSVTAAD | | | |
| | | | | | | | | | | | | | | | | AAGCTGAGCTCTGTGACCGCCGCGGAC | | TGVYYCVRDR | | | |
| | | | | | | | | | | | | | | | | ACGGGCGTGTACTACTGTGTGAGAGAT | | SLYCSNGNCP | | | |
| | | | | | | | | | | | | | | | | CGGTCCTTATATTGTAGTAATGGTAAC | | HMDVWGKGTT | | | |
| | | | | | | | | | | | | | | | | TGCCCACACATGGACGTCTGGGGCAAA | | VTVSS | | | |
| | | | | | | | | | | | | | | | | GGGACCACGGTCACCGTCTCCTCA | | | | | |
| ADI- | IGHV3- | RCTLQQSG | 62 | FTFSSYG | 262 | WVRQA | 456 | VVWYDG | 587 | RFTIS | 777 | ARDTWE | 1088 | WGKGTT | 1291 | AGGTGCACGCTGCAGCAGTCTGGGGGA | 1377 | RCTLQQSGGG | 1660 | | |
| 75785 | 33 | GGVVQPGK | | MH | | PGQGL | | SNKYYV | | RDNSK | | VPAADP | | VTVSS | | GGCGTGGTCCAGCCTGGGAAGTCCCTG | | VVQPGKSLRL | | | |
| | | SLRLSCAA | | | | EWVA | | DSVKG | | NTLYL | | PYYYYY | | | | AGACTCTCCTGTGCAGCGTCTGGATTC | | SCAASGFTFS | | | |
| | | SG | | | | | | | | QMNSL | | MDV | | | | ACCTTCAGTAGCTATGGCATGCACTGG | | SYGMHWVRQA | | | |
| | | | | | | | | | | RAEDT | | | | | | GTCCGCCAGGCTCCAGGCCAGGGGCTG | | PGQGLEWVAV | | | |
| | | | | | | | | | | AVYYC | | | | | | GAGTGGGTGGCAGTTGTATGGTATGAT | | VWYDGSNKYY | | | |
| | | | | | | | | | | | | | | | | GGAAGTAATAAATATTATGTAGACTCC | | VDSVKGRFTI | | | |
| | | | | | | | | | | | | | | | | GTGAAGGGCCGATTCACCATCTCCAGA | | SRDNSKNTLY | | | |
| | | | | | | | | | | | | | | | | GATAATTCCAAGAACACGCTGTATCTG | | LQMNSLRAED | | | |
| | | | | | | | | | | | | | | | | CAAATGAATAGCCTGAGAGCCGAGGAC | | TAVYYCARDT | | | |
| | | | | | | | | | | | | | | | | ACGGCTGTGTATTACTGTGCGAGAGAT | | WEVPAADPPY | | | |
| | | | | | | | | | | | | | | | | ACTTGGGAAGTACCAGCTGCCGACCCT | | YYYYMDVWGK | | | |
| | | | | | | | | | | | | | | | | CCCTACTACTACTACTATATGGACGTC | | GTTVTVSS | | | |
| | | | | | | | | | | | | | | | | TGGGGCAAAGGGACCACGGTCACCGTC | | | | | |
| | | | | | | | | | | | | | | | | TCCTCA | | | | | |
| ADI- | IGHV4- | QVQLQQSG | 44 | GSISGIN | 257 | WVRQS | 455 | EISQSG | 582 | RVTIS | 838 | ARVSGF | 1083 | WGQGTL | 1286 | CAGGTGCAGCTGCAGCAGTCGGGCCCA | 1371 | QVQLQQSGPG | 1654 | | |
| 75594 | 4 | PGLVKPSG | | WWS | | PRQGL | | STYYNP | | VDKSK | | FDS | | VTVSS | | GGACTGGTGAAGCCTTCGGGGACCCTG | | LVKPSGTLSL | | | |
| | | TLSLTCAV | | | | EWIG | | SLKS | | NQFSL | | | | | | TCCCTCACCTGCGCTGTCTCTGGTGGC | | TCAVSGGSIS | | | |
| | | SG | | | | | | | | KLTSV | | | | | | TCCATCAGTGGTATAAACTGGTGGAGT | | GINWWSWVRQ | | | |
| | | | | | | | | | | TAADT | | | | | | TGGGTCCGCCAGTCCCCACGGCAGGGG | | SPRQGLEWIG | | | |
| | | | | | | | | | | AIYYC | | | | | | CTGGAGTGGATTGGGGAAATCTCTCAG | | EISQSGSTYY | | | |
| | | | | | | | | | | | | | | | | AGTGGGAGCACCTACTACAACCCGTCT | | NPSLKSRVTI | | | |
| | | | | | | | | | | | | | | | | CTCAAAAGTCGAGTCACCATATCAGTA | | SVDKSKNQFS | | | |
| | | | | | | | | | | | | | | | | GACAAGTCTAAGAACCAGTTCTCCCTG | | LKLTSVTAAD | | | |
| | | | | | | | | | | | | | | | | AAGCTGACCTCTGTGACCGCCGCGGAC | | TAIYYCARVS | | | |
| | | | | | | | | | | | | | | | | ACGGCCATATATTACTGTGCGAGAGTC | | GFFDSWGQGT | | | |
| | | | | | | | | | | | | | | | | AGCGGGTTTTTTGACTCCTGGGGCCAG | | LVTVSS | | | |
| | | | | | | | | | | | | | | | | GGAACCCTGGTCACCGTCTCCTCA | | | | | |
| ADI- | IGHV4- | QVQLQQSG | 56 | GSISSYY | 263 | WIRQP | 457 | YIYYSG | 588 | RVTMS | 844 | ARHAPI | 1089 | WGQGTL | 1286 | CAGGTGCAGCTGCAGCAGTCGGGCCCA | 1378 | QVQLQQSGPG | 1661 | | |
| 75801 | 59 | PGLVKPSQ | | WS | | PGKGL | | NTNYSP | | VDTSK | | HSALYE | | VTVSS | | GGACTGGTGAAGCCTTCGCAGACCCTG | | LVKPSQTLSL | | | |
| | | TLSLTCTV | | | | QWIG | | SLRS | | NQFSL | | PNTYFD | | | | TCCCTCACCTGCACTGTCTCTGGTGGC | | TCTVSGGSIS | | | |
| | | SG | | | | | | | | KLSSV | | Y | | | | TCCATCAGTAGTTACTACTGGAGCTGG | | SYYWSWIRQP | | | |
| | | | | | | | | | | TAADT | | | | | | ATCCGGCAGCCCCCAGGGAAGGGACTG | | PGKGLQWIGY | | | |
| | | | | | | | | | | AVYYC | | | | | | CAGTGGATTGGGTATATCTATTACAGT | | IYYSGNTNYS | | | |
| | | | | | | | | | | | | | | | | GGGAACACCAACTACAGCCCCTCCCTC | | PSLRSRVTMS | | | |
| | | | | | | | | | | | | | | | | AGGAGTCGAGTCACCATGTCAGTAGAC | | VDTSKNQFSL | | | |
| | | | | | | | | | | | | | | | | ACGTCCAAGAATCAGTTCTCCCTGAAA | | KLSSVTAADT | | | |
| | | | | | | | | | | | | | | | | CTGAGCTCTGTGACCGCCGCAGACACG | | AVYYCARHAP | | | |
| | | | | | | | | | | | | | | | | GCCGTGTATTACTGTGCGAGACATGCT | | IHSALYEPNT | | | |
| | | | | | | | | | | | | | | | | CCGATACATTCGGCACTTTACGAACCG | | YFDYWGQGTL | | | |
| | | | | | | | | | | | | | | | | AATACGTACTTTGACTACTGGGGCCAG | | VTVSS | | | |
| | | | | | | | | | | | | | | | | GGAACCCTGGTCACCGTCTCCTCA | | | | | |
| ADI- | IGHV3- | RCTLRAVW | 63 | FTFEDYA | 264 | WVRLL | 458 | GVSWNS | 589 | RFIIS | 845 | VKLYSG | 1090 | WGQGTT | 1287 | AGGTGCACGCTGCGTGCAGTCTGGGGG | 1379 | RCTLRAVWGR | 1662 | | |
| 75624 | 9 | GRLGTAWQ | | IH | | PGKGL | | GVIDYV | | RDNAK | | GRDYDF | | VTVSS | | AGGCTTGGTACAGCCTGGCAGGTCCTG | | LGTAWQVLTL | | | |
| | | VLTLSCTA | | | | EWVS | | DSVKG | | NSLYL | | RSGSQN | | | | ACACTCTCCTGTACAGCCTCTGGATTC | | SCTASGFTFE | | | |
| | | SG | | | | | | | | HMRSL | | GNGNGM | | | | ACGTTTGAAGATTATGCCATACACTGG | | DYAIHWVRLL | | | |
| | | | | | | | | | | RPEDT | | DV | | | | GTCCGGCTACTTCCAGGGAAGGGCCTG | | PGKGLEWVSG | | | |
| | | | | | | | | | | ALYYC | | | | | | GAGTGGGTCTCGGGTGTTAGTTGGAAT | | VSWNSGVIDY | | | |
| | | | | | | | | | | | | | | | | AGTGGTGTCATAGACTACGTGGACTCT | | VDSVKGRFII | | | |
| | | | | | | | | | | | | | | | | GTGAAGGGCCGATTCATCATCTCCAGA | | SRDNAKNSLY | | | |
| | | | | | | | | | | | | | | | | GACAACGCCAAGAACTCCTTATATCTG | | LHMRSLRPED | | | |
| | | | | | | | | | | | | | | | | CATATGAGAAGTCTGAGACCTGAGGAC | | TALYYCVKLY | | | |
| | | | | | | | | | | | | | | | | ACGGCCTTATATTACTGTGTAAAATTA | | SGGRDYDFRS | | | |
| | | | | | | | | | | | | | | | | TACTCGGGGGGGAGGGACTACGATTTT | | GSQNGNGNGM | | | |
| | | | | | | | | | | | | | | | | CGGAGTGGTTCGCAAAATGGGAATGGA | | DVWGQGTTVT | | | |
| | | | | | | | | | | | | | | | | AACGGTATGGACGTCTGGGGCCAAGGG | | VSS | | | |
| | | | | | | | | | | | | | | | | ACCACGGTCACCGTCTCCTCA | | | | | |
| ADI- | IGHV3- | QVQLQESG | 64 | FNFGNYV | 265 | WVRQA | 432 | LIRSNS | 590 | RFTIS | 846 | STSRGG | 1091 | WGQGTL | 1286 | CAGGTGCAGCTGCAGGAGTCTGGGGGA | 1380 | QVQLQESGGG | 1663 | | |
| 75764 | 49 | GGLVQPGR | | MS | | PGKGL | | FGGTTQ | | RDDST | | STNWFF | | VTVSS | | GGCTTGGTACAGCCAGGGCGGTCCCTG | | LVQPGRSLRL | | | |
| | | SLRLSCTG | | | | EWVG | | YAASVR | | SIAYL | | Y | | | | AGACTCTCCTGTACAGGTTCTGGATTC | | SCTGSGFNFG | | | |
| | | SG | | | | | | G | | QMNSL | | | | | | AACTTTGGTAATTATGTTATGAGCTGG | | NYVMSWVRQA | | | |
| | | | | | | | | | | KTEDT | | | | | | GTCCGCCAGGCTCCAGGGAAGGGGCTG | | PGKGLEWVGL | | | |
| | | | | | | | | | | AVYYC | | | | | | GAGTGGGTTGGTCTCATTAGAAGCAAT | | IRSNSFGGTT | | | |
| | | | | | | | | | | | | | | | | TCTTTTGGTGGGACAACACAATACGCC | | QYAASVRGRF | | | |
| | | | | | | | | | | | | | | | | GCGTCTGTGAGAGGCAGATTCACCATC | | TISRDDSTSI | | | |
| | | | | | | | | | | | | | | | | TCAAGAGATGATTCCACAAGCATCGCC | | AYLQMNSLKT | | | |
| | | | | | | | | | | | | | | | | TATCTGCAAATGAACAGCCTGAAAACC | | EDTAVYYCST | | | |
| | | | | | | | | | | | | | | | | GAAGACACAGCCGTATATTACTGTTCC | | SRGGSTNWFF | | | |
| | | | | | | | | | | | | | | | | ACTAGTCGTGGGGGTAGCACCAACTGG | | YWGQGTLVTV | | | |
| | | | | | | | | | | | | | | | | TTTTTTTACTGGGGCCAGGGAACCCTG | | SS | | | |
| | | | | | | | | | | | | | | | | GTCACCGTCTCCTCA | | | | | |
| ADI- | IGHV3- | EVQLVQSG | 65 | FTFSSYA | 266 | WVRQA | 459 | SISSDG | 591 | RFTIS | 847 | VKDLGT | 1092 | WGQGTT | 1287 | GAGGTGCAGCTGGTGCAGTCTGGGGGA | 1381 | EVQLVQSGGG | 1664 | | |
| 75806 | 64D | GGLVQPGG | | VY | | PGKGP | | GITYYA | | RDNSK | | MIVDVL | | VTVSS | | GGCTTGGTCCAGCCGGGGGGGTCCCTG | | LVQPGGSLRL | | | |
| | | SLRLSCSA | | | | EYVS | | DSVKG | | NTLYL | | EM | | | | AGACTCTCCTGTTCAGCCTCTGGATTC | | SCSASGFTFS | | | |
| | | SG | | | | | | | | QMSTL | | | | | | ACCTTCAGTAGTTATGCTGTGTACTGG | | SYAVYWVRQA | | | |
| | | | | | | | | | | RAEDT | | | | | | GTCCGCCAGGCTCCAGGGAAGGGACCG | | PGKGPEYVSS | | | |
| | | | | | | | | | | AVYYC | | | | | | GAATATGTTTCATCTATTAGTAGTGAT | | ISSDGGITYY | | | |
| | | | | | | | | | | | | | | | | GGGGGTATCACATACTACGCAGACTCA | | ADSVKGRFTI | | | |
| | | | | | | | | | | | | | | | | GTGAAGGGCAGATTCACCATCTCCAGA | | SRDNSKNTLY | | | |
| | | | | | | | | | | | | | | | | GACAATTCCAAGAACACGCTGTATCTT | | LQMSTLRAED | | | |
| | | | | | | | | | | | | | | | | CAAATGAGCACTCTGAGAGCTGAGGAC | | TAVYYCVKDL | | | |
| | | | | | | | | | | | | | | | | ACGGCTGTGTATTATTGTGTGAAAGAT | | GTMIVDVLEM | | | |
| | | | | | | | | | | | | | | | | CTAGGAACTATGATAGTAGATGTTTTG | | WGQGTTVTVS | | | |
| | | | | | | | | | | | | | | | | GAAATGTGGGGCCAAGGGACCACGGTC | | S | | | |
| | | | | | | | | | | | | | | | | ACCGTCTCCTCA | | | | | |
| ADI- | IGHV3- | RSTLVQSG | 66 | FIFSRQW | 267 | WVRQA | 431 | SIKQDG | 592 | RFTIS | 848 | ARETGA | 1093 | RGQGTL | 1292 | AGGTCCACGCTGGTGCAGTCTGGGGGA | 1382 | RSTLVQSGGG | 1665 | | |
| 75630 | 7 | GGLVQPGG | | MS | | PGKGL | | SEKYYV | | RDNAK | | FWFDP | | VTVSS | | GGCCTGGTCCAGCCTGGGGGGTCCCTG | | LVQPGGSLRL | | | |
| | | SLRLSCAA | | | | EWVA | | DSVKG | | NSLYL | | | | | | AGACTCTCCTGTGCAGCCTCTGGATTC | | SCAASGFIFS | | | |
| | | SG | | | | | | | | QLNSL | | | | | | ATCTTTAGTAGACAATGGATGAGCTGG | | RQWMSWVRQA | | | |
| | | | | | | | | | | RVEDT | | | | | | GTCCGCCAGGCTCCAGGGAAGGGACTG | | PGKGLEWVAS | | | |
| | | | | | | | | | | AVYYC | | | | | | GAGTGGGTGGCCAGCATAAAGCAAGAT | | IKQDGSEKYY | | | |
| | | | | | | | | | | | | | | | | GGGAGTGAGAAATACTATGTGGACTCT | | VDSVKGRFTI | | | |
| | | | | | | | | | | | | | | | | GTGAAGGGCCGATTCACCATCTCCAGA | | SRDNAKNSLY | | | |
| | | | | | | | | | | | | | | | | GACAACGCCAAGAACTCCCTGTATCTG | | LQLNSLRVED | | | |
| | | | | | | | | | | | | | | | | CAATTGAACAGCCTGAGAGTCGAAGAC | | TAVYYCARET | | | |
| | | | | | | | | | | | | | | | | ACGGCTGTGTATTACTGTGCGAGAGAA | | GAFWFDPRGQ | | | |
| | | | | | | | | | | | | | | | | ACGGGTGCATTCTGGTTCGACCCCAGG | | GTLVTVSS | | | |
| | | | | | | | | | | | | | | | | GGCCAGGGAACCCTGGTCACCGTCTCC | | | | | |
| | | | | | | | | | | | | | | | | TCA | | | | | |
| ADI- | IGHV4- | QVQLQESG | 67 | GSIRSYY | 268 | WIRQP | 445 | YVHDSG | 593 | RVTMS | 849 | ASLLVR | 1094 | WGQGTL | 1286 | CAGGTGCAGCTGCAGGAGTCGGGCCCA | 1383 | QVQLQESGPG | 1666 | | |
| 75609 | 59 | PGLVKPSE | | WS | | PGKGL | | STNYNP | | LDTSK | | YRGYEI | | VTVSS | | GGACTGGTGAAGCCTTCGGAGACCCTG | | LVKPSETLSL | | | |
| | | TLSLTCTV | | | | EWIG | | SLKS | | NQFSL | | DY | | | | TCCCTCACCTGCACTGTTTCTGGTGGC | | TCTVSGGSIR | | | |
| | | SG | | | | | | | | KLRSV | | | | | | TCCATCAGGAGTTACTACTGGAGCTGG | | SYYWSWIRQP | | | |
| | | | | | | | | | | TAADT | | | | | | ATCCGGCAGCCCCCAGGGAAGGGACTG | | PGKGLEWIGY | | | |
| | | | | | | | | | | AVYYC | | | | | | GAGTGGATTGGATATGTCCACGACAGT | | VHDSGSTNYN | | | |
| | | | | | | | | | | | | | | | | GGGAGCACCAACTACAACCCCTCCCTT | | PSLKSRVTMS | | | |
| | | | | | | | | | | | | | | | | AAGAGTCGAGTCACCATGTCACTAGAC | | LDTSKNQFSL | | | |
| | | | | | | | | | | | | | | | | ACGTCCAAGAATCAGTTCTCCCTGAAG | | KLRSVTAADT | | | |
| | | | | | | | | | | | | | | | | CTCAGGTCTGTGACCGCTGCGGACACG | | AVYYCASLLV | | | |
| | | | | | | | | | | | | | | | | GCCGTGTATTATTGTGCGAGTCTGTTA | | RYRGYEIDYW | | | |
| | | | | | | | | | | | | | | | | GTCCGATATAGAGGATACGAAATTGAC | | GQGTLVTVSS | | | |
| | | | | | | | | | | | | | | | | TATTGGGGCCAGGGAACCCTGGTCACC | | | | | |
| | | | | | | | | | | | | | | | | GTCTCCTCA | | | | | |
| ADI- | IGHV3- | QVQLVESG | 68 | FIFSDYS | 227 | WVRQA | 460 | GISSNG | 594 | RFTIS | 850 | VKDMGS | 1095 | WGQGTT | 1287 | CAGGTGCAGCTGGTGGAGTCTGGGGGA | 1384 | QVQLVESGGG | 1667 | | |
| 75671 | 64D | GGLVQPGG | | MH | | PGKGL | | DITDYA | | RDNSK | | SKYWDV | | VTVSS | | GGCTTGGTCCAGCCTGGGGGGTCCCTG | | LVQPGGSLSL | | | |
| | | SLSLSCVV | | | | EYLS | | DSVKD | | NTLYL | | FDV | | | | AGCCTCTCCTGTGTAGTCTCTGGATTC | | SCVVSGFIFS | | | |
| | | SG | | | | | | | | QMSSM | | | | | | ATCTTCAGTGACTATTCTATGCACTGG | | DYSMHWVRQA | | | |
| | | | | | | | | | | RIEEW | | | | | | GTCCGCCAGGCTCCAGGGAAGGGACTG | | PGKGLEYLSG | | | |
| | | | | | | | | | | AVYHC | | | | | | GAATATCTTTCAGGAATTAGTAGTAAT | | ISSNGDITDY | | | |
| | | | | | | | | | | | | | | | | GGGGATATCACAGACTACGCAGACTCA | | ADSVKDRFTI | | | |
| | | | | | | | | | | | | | | | | GTGAAGGACAGATTCACCATCTCCAGA | | SRDNSKNTLY | | | |
| | | | | | | | | | | | | | | | | GACAATTCCAAGAACACACTGTATCTT | | LQMSSMRIEE | | | |
| | | | | | | | | | | | | | | | | CAAATGAGCAGTATGAGAATTGAAGAA | | WAVYHCVKDM | | | |
| | | | | | | | | | | | | | | | | TGGGCTGTTTATCATTGTGTGAAAGAT | | GSSKYWDVFD | | | |
| | | | | | | | | | | | | | | | | ATGGGGAGCTCGAAGTACTGGGATGTT | | VWGQGTTVTV | | | |
| | | | | | | | | | | | | | | | | TTTGATGTTTGGGGCCAAGGGACCACG | | SS | | | |
| | | | | | | | | | | | | | | | | GTCACCGTCTCCTCA | | | | | |
| ADI- | IGHV3- | QVQLVQSG | 69 | VTVSKYA | 269 | WVRQA | 437 | GISGYG | 595 | RYTIS | 851 | AKGPRS | 1096 | WGRGTL | 1288 | CAGGTGCAGCTGGTGCAGTCTGGGGGA | 1385 | QVQLVQSGGG | 1668 | | |
| 75660 | 23 | GGLVQPGG | | MS | | PGKGL | | DTTYYA | | RDNSK | | NHDYFD | | VTVSS | | GGCTTGGTACAGCCTGGGGGGTCCGTG | | LVQPGGSVRL | | | |
| | | SVRLSCGA | | | | EWVS | | DSVKG | | NTLYL | | D | | | | AGACTCTCCTGTGGAGCCTCTGGAGTC | | SCGASGVTVS | | | |
| | | SG | | | | | | | | QMNSL | | | | | | ACAGTTAGCAAATATGCCATGAGCTGG | | KYAMSWVRQA | | | |
| | | | | | | | | | | RDEDT | | | | | | GTCCGCCAGGCTCCAGGGAAGGGGCTG | | PGKGLEWVSG | | | |
| | | | | | | | | | | AVYYC | | | | | | GAGTGGGTCTCAGGTATTAGTGGTTAT | | ISGYGDTTYY | | | |
| | | | | | | | | | | | | | | | | GGTGATACCACATACTACGCAGACTCC | | ADSVKGRYTI | | | |
| | | | | | | | | | | | | | | | | GTGAAGGGCCGGTACACCATCTCCAGA | | SRDNSKNTLY | | | |
| | | | | | | | | | | | | | | | | GACAATTCCAAGAACACGCTGTATCTG | | LQMNSLRDED | | | |
| | | | | | | | | | | | | | | | | CAAATGAACAGCCTGAGAGACGAGGAC | | TAVYYCAKGP | | | |
| | | | | | | | | | | | | | | | | ACGGCCGTATATTACTGTGCGAAAGGA | | RSNHDYFDDW | | | |
| | | | | | | | | | | | | | | | | CCCCGCAGTAACCACGACTACTTTGAC | | GRGTLVTVSS | | | |
| | | | | | | | | | | | | | | | | GATTGGGGCCGGGGAACCCTGGTCACC | | | | | |
| | | | | | | | | | | | | | | | | GTCTCCTCA | | | | | |
| ADI- | IGHV3- | QVQLVQSG | 70 | FSFSTYG | 270 | WIRQA | 461 | VIWSGG | 596 | RFTIS | 852 | ARDGDG | 1097 | WGQGTL | 1286 | CAGGTGCAGCTGGTGCAGTCTGGGGGA | 1386 | QVQLVQSGGG | 1669 | | |
| 75802 | 33 | GGVVQPGR | | MH | | PGKGL | | SGKHYA | | RDNSK | | GLVTFP | | VTVSS | | GGCGTGGTCCAGCCTGGGAGGTCCCTG | | VVQPGRSLRL | | | |
| | | SLRLSCTA | | | | EWVA | | DSVKG | | NTLNL | | DY | | | | AGACTCTCCTGTACAGCGTCTGGATTC | | SCTASGFSFS | | | |
| | | SG | | | | | | | | QMNSL | | | | | | AGCTTCAGTACCTATGGCATGCACTGG | | TYGMHWIRQA | | | |
| | | | | | | | | | | RVEDT | | | | | | ATTCGCCAGGCTCCAGGCAAGGGGCTG | | PGKGLEWVAV | | | |
| | | | | | | | | | | ATYYC | | | | | | GAGTGGGTGGCAGTTATCTGGTCTGGT | | IWSGGSGKHY | | | |
| | | | | | | | | | | | | | | | | GGAAGTGGTAAACACTATGCAGACTCC | | ADSVKGRFTI | | | |
| | | | | | | | | | | | | | | | | GTGAAGGGCCGGTTCACCATCTCCAGA | | SRDNSKNTLN | | | |
| | | | | | | | | | | | | | | | | GATAATTCGAAGAACACACTGAATCTG | | LQMNSLRVED | | | |
| | | | | | | | | | | | | | | | | CAAATGAACAGCCTGAGAGTCGAGGAC | | TATYYCARDG | | | |
| | | | | | | | | | | | | | | | | ACGGCTACATATTACTGTGCGAGAGAT | | DGGLVTFPDY | | | |
| | | | | | | | | | | | | | | | | GGCGACGGGGGGCTGGTTACCTTCCCG | | WGQGTLVTVS | | | |
| | | | | | | | | | | | | | | | | GACTACTGGGGCCAGGGAACCCTGGTC | | S | | | |
| | | | | | | | | | | | | | | | | ACCGTCTCCTCA | | | | | |
| ADI- | IGHV2- | QVTLKESG | 71 | FSLSTTG | 271 | WIRQP | 441 | LIYWDD | 597 | RLSIT | 853 | AHTPDG | 1098 | WGQGTL | 1286 | CAGGTCACGCTGAAGGAGTCTGGTCCT | 1387 | QVTLKESGPT | 1670 | | |
| 75614 | 5 | PTLVKPTQ | | VGVG | | PGKAL | | DQRYSP | | KDTSK | | RYFDWL | | VTVSS | | ACCCTGGTGAAACCCACACAGACCCTC | | LVKPTQTLTL | | | |
| | | TLTLTCTF | | | | EWLA | | SLKS | | NQVVL | | TPLFGN | | | | ACGCTGACCTGCACCTTCTCTGGTTTC | | TCTFSGFSLS | | | |
| | | SG | | | | | | | | TMTNM | | YYFDF | | | | TCACTCAGCACTACTGGAGTGGGTGTG | | TTGVGVGWIR | | | |
| | | | | | | | | | | DPVDT | | | | | | GGCTGGATCCGTCAGCCCCCAGGAAAG | | QPPGKALEWL | | | |
| | | | | | | | | | | ATYYC | | | | | | GCCCTGGAGTGGCTTGCGCTCATTTAT | | ALIYWDDDQR | | | |
| | | | | | | | | | | | | | | | | TGGGATGATGATCAGCGCTACAGCCCA | | YSPSLKSRLS | | | |
| | | | | | | | | | | | | | | | | TCTCTGAAGAGCAGGCTCAGCATCACC | | ITKDTSKNQV | | | |
| | | | | | | | | | | | | | | | | AAGGACACCTCCAAAAACCAGGTGGTC | | VLTMTNMDPV | | | |
| | | | | | | | | | | | | | | | | CTTACAATGACCAACATGGACCCTGTG | | DTATYYCAHT | | | |
| | | | | | | | | | | | | | | | | GACACAGCCACATACTACTGTGCACAC | | PDGRYFDWLT | | | |
| | | | | | | | | | | | | | | | | ACCCCGGACGGGCGATATTTTGACTGG | | PLFGNYYFDF | | | |
| | | | | | | | | | | | | | | | | TTAACCCCTCTTTTTGGAAACTACTAC | | WGQGTLVTVS | | | |
| | | | | | | | | | | | | | | | | TTTGACTTCTGGGGCCAGGGAACCCTG | | S | | | |
| | | | | | | | | | | | | | | | | GTCACCGTCTCCTCA | | | | | |
| ADI- | IGHV3- | QVQLVQSG | 12 | FTFSRYW | 272 | WVRQA | 431 | NIKQDG | 598 | RFTIS | 854 | AKDSTP | 1099 | WGQGTL | 1286 | CAGGTGCAGCTGGTGCAGTCTGGGGGA | 1388 | QVQLVQSGGG | 1671 | | |
| 75661 | 7 | GGLVQPGG | | MS | | PGKGL | | SEKNYV | | RDNAK | | ATYSND | | VTVSS | | GGCTTGGTCCAGCCGGGGGGGTCCCTG | | LVQPGGSLRL | | | |
| | | SLRLSCAA | | | | EWVA | | DSVKG | | NSLYL | | WTNAEY | | | | AGACTCTCCTGTGCAGCCTCTGGATTC | | SCAASGFTFS | | | |
| | | SG | | | | | | | | QMNSL | | FQH | | | | ACCTTTAGTCGTTATTGGATGAGCTGG | | RYWMSWVRQA | | | |
| | | | | | | | | | | RAEDT | | | | | | GTCCGCCAGGCTCCAGGGAAGGGGCTG | | PGKGLEWVAN | | | |
| | | | | | | | | | | AVYYC | | | | | | GAGTGGGTGGCCAACATTAAGCAAGAT | | IKQDGSEKNY | | | |
| | | | | | | | | | | | | | | | | GGAAGTGAGAAGAACTATGTGGACTCT | | VDSVKGRFTI | | | |
| | | | | | | | | | | | | | | | | GTGAAGGGCCGATTCACCATCTCCAGA | | SRDNAKNSLY | | | |
| | | | | | | | | | | | | | | | | GACAACGCCAAGAACTCACTGTATCTG | | LQMNSLRAED | | | |
| | | | | | | | | | | | | | | | | CAAATGAATAGTCTGAGAGCCGAGGAC | | TAVYYCAKDS | | | |
| | | | | | | | | | | | | | | | | ACGGCTGTGTATTACTGTGCGAAAGAT | | TPATYSNDWT | | | |
| | | | | | | | | | | | | | | | | TCTACGCCGGCTACTTATAGCAATGAC | | NAEYFQHWGQ | | | |
| | | | | | | | | | | | | | | | | TGGACAAACGCTGAGTACTTCCAGCAC | | GTLVTVSS | | | |
| | | | | | | | | | | | | | | | | TGGGGCCAGGGCACCCTGGTCACCGTC | | | | | |
| | | | | | | | | | | | | | | | | TCCTCA | | | | | |
| ADI- | IGHV4- | QVQLQQSG | 72 | GFINSGG | 273 | WIRQH | 462 | SIFDSG | 599 | RLTIS | 855 | ARSPGG | 1081 | WGQGTL | 1286 | CAGGTGCAGCTGCAGCAGTCGGGCCCA | 1389 | QVQLQQSGPG | 1672 | | |
| 75697 | 31 | PGLVKPSQ | | NYWS | | PGTGL | | TTYYNP | | VDTSK | | LFDF | | VTVSS | | GGACTGGTGAAGCCTTCACAGACCCTG | | LVKPSQTLSL | | | |
| | | TLSLTCSV | | | | EWIG | | SLKS | | NQFSL | | | | | | TCCCTAACCTGCAGTGTCTCTGGTGGC | | TCSVSGGFIN | | | |
| | | SG | | | | | | | | KLSSV | | | | | | TTCATCAACAGCGGTGGGAACTACTGG | | SGGNYWSWIR | | | |
| | | | | | | | | | | TAADT | | | | | | AGCTGGATCCGCCAGCACCCAGGGACG | | QHPGTGLEWI | | | |
| | | | | | | | | | | AVYFC | | | | | | GGCCTGGAGTGGATTGGGTCCATCTTT | | GSIFDSGTTY | | | |
| | | | | | | | | | | | | | | | | GACAGTGGGACCACCTACTACAACCCG | | YNPSLKSRLT | | | |
| | | | | | | | | | | | | | | | | TCCCTCAAGAGTCGACTCACTATATCA | | ISVDTSKNQF | | | |
| | | | | | | | | | | | | | | | | GTAGACACGTCTAAGAACCAGTTCTCC | | SLKLSSVTAA | | | |
| | | | | | | | | | | | | | | | | CTGAAGCTGAGCTCTGTGACTGCCGCG | | DTAVYFCARS | | | |
| | | | | | | | | | | | | | | | | GACACGGCCGTGTATTTCTGTGCGAGG | | PGGLFDFWGQ | | | |
| | | | | | | | | | | | | | | | | TCCCCTGGGGGATTATTTGACTTCTGG | | GTLVTVSS | | | |
| | | | | | | | | | | | | | | | | GGCCAGGGAACCCTGGTCACCGTCTCC | | | | | |
| | | | | | | | | | | | | | | | | TCA | | | | | |
| ADI- | IGHV4- | QVQLQQWG | 73 | GSFSDYF | 274 | WIRQP | 445 | ELTHTG | 600 | RVTIL | 856 | ARGDFY | 1100 | WGQGTT | 1287 | CAGGTGCAGCTACAGCAGTGGGGCGCA | 1390 | QVQLQQWGAG | 1673 | | |
| 75700 | 34 | AGLLKPSE | | WN | | PGKGL | | STNYNP | | ADTSK | | STRLAF | | VTVSS | | GGACTGTTGAAGCCCTCGGAGACCCTG | | LLKPSETLSL | | | |
| | | TLSLTCGV | | | | EWIG | | SLKS | | SQFSL | | DL | | | | TCCCTCACCTGCGGTGTCTATGATGGG | | TCGVYDGSFS | | | |
| | | YD | | | | | | | | NLSSV | | | | | | TCCTTCAGTGATTACTTCTGGAACTGG | | DYFWNWIRQP | | | |
| | | | | | | | | | | TAADT | | | | | | ATCCGCCAGCCCCCAGGGAAGGGGCTG | | PGKGLEWIGE | | | |
| | | | | | | | | | | AVYYC | | | | | | GAGTGGATTGGGGAGCTCACGCATACT | | LTHTGSTNYN | | | |
| | | | | | | | | | | | | | | | | GGAAGTACCAACTACAACCCGTCCCTC | | PSLKSRVTIL | | | |
| | | | | | | | | | | | | | | | | AAGAGTCGAGTCACCATATTAGCAGAC | | ADTSKSQFSL | | | |
| | | | | | | | | | | | | | | | | ACGTCCAAGAGCCAGTTCTCCCTGAAC | | NLSSVTAADT | | | |
| | | | | | | | | | | | | | | | | CTGAGCTCTGTGACCGCCGCGGACACG | | AVYYCARGDF | | | |
| | | | | | | | | | | | | | | | | GCTGTGTATTACTGTGCGAGAGGCGAT | | YSTRLAFDLW | | | |
| | | | | | | | | | | | | | | | | TTTTATAGCACCAGGCTCGCTTTTGAT | | GQGTTVTVSS | | | |
| | | | | | | | | | | | | | | | | CTCTGGGGCCAAGGGACCACGGTCACC | | | | | |
| | | | | | | | | | | | | | | | | GTCTCCTCA | | | | | |
| ADI- | IGHV3- | QVQLLESG | 74 | FTLSNDD | 275 | WVRQP | 463 | GTGTSG | 601 | RFTIS | 857 | VRGTAT | 1101 | WGKGTT | 1291 | CAGGTGCAGCTGCTGGAGTCTGGGGGA | 1391 | QVQLLESGGG | 1674 | | |
| 75787 | 13 | GGLVQPGG | | MH | | IGKGL | | DTYYLD | | RENAR | | GYYYYM | | VTVSS | | GGCTTGGTACAGCCTGGGGGGTCCCTG | | LVQPGGSLRL | | | |
| | | SLRLSCAA | | | | EWVS | | SVKG | | NSVYL | | DV | | | | AGACTCTCCTGTGCAGCCTCTGGATTC | | SCAASGFTLS | | | |
| | | SG | | | | | | | | QMNSL | | | | | | ACCCTCAGTAACGACGACATGCACTGG | | NDDMHWVRQP | | | |
| | | | | | | | | | | GAGDT | | | | | | GTCCGCCAACCTATAGGAAAAGGTCTG | | IGKGLEWVSG | | | |
| | | | | | | | | | | AIYYC | | | | | | GAGTGGGTCTCAGGTACTGGTACTTCT | | TGTSGDTYYL | | | |
| | | | | | | | | | | | | | | | | GGTGACACATATTATCTAGACTCCGTG | | DSVKGRFTIS | | | |
| | | | | | | | | | | | | | | | | AAGGGCCGATTCACCATCTCCAGAGAA | | RENARNSVYL | | | |
| | | | | | | | | | | | | | | | | AATGCCAGGAACTCCGTGTATCTTCAA | | QMNSLGAGDT | | | |
| | | | | | | | | | | | | | | | | ATGAACAGTCTGGGAGCCGGGGACACG | | AIYYCVRGTA | | | |
| | | | | | | | | | | | | | | | | GCTATATATTATTGTGTAAGAGGGACT | | TGYYYYMDVW | | | |
| | | | | | | | | | | | | | | | | GCAACGGGCTACTACTACTACATGGAC | | GKGTTVTVSS | | | |
| | | | | | | | | | | | | | | | | GTCTGGGGCAAAGGGACCACGGTCACC | | | | | |
| | | | | | | | | | | | | | | | | GTCTCCTCA | | | | | |
| ADI- | IGHV3- | QVHAGGVW | 75 | FTFSSYG | 262 | WVRQA | 431 | VISYDG | 602 | RFTIS | 858 | VRDPQD | 1102 | WGQGTL | 1286 | CAGGTGCACGCTGGTGGAGTCTGGGGG | 1392 | QVHAGGVWGR | 1675 | | |
| 75642 | 30 | GRRGPAGR | | MH | | PGKGL | | SNKYYR | | RETSK | | CGGSRC | | VTVSS | | AGGCGTGGTCCAGCTGGGAGGTCCCTG | | RGPAGRSLRL | | | |
| | | SLRLSCAA | | | | EWVA | | DSVKG | | NTLYL | | SNALGY | | | | AGACTCTCCTGTGCAGCCTCTGGATTC | | SCAASGFTFS | | | |
| | | SG | | | | | | | | QMTSL | | FDY | | | | ACCTTCAGTAGCTATGGAATGCACTGG | | SYGMHWVRQA | | | |
| | | | | | | | | | | RAEDT | | | | | | GTCCGCCAGGCTCCAGGCAAGGGCCTA | | PGKGLEWVAV | | | |
| | | | | | | | | | | AVYYC | | | | | | GAGTGGGTGGCAGTTATATCATATGAT | | ISYDGSNKYY | | | |
| | | | | | | | | | | | | | | | | GGAAGTAATAAATACTACAGAGACTCC | | RDSVKGRFTI | | | |
| | | | | | | | | | | | | | | | | GTGAAGGGCCGATTCACCATCTCCAGA | | SRETSKNTLY | | | |
| | | | | | | | | | | | | | | | | GAAACTTCCAAGAACACGCTGTATCTG | | LQMTSLRAED | | | |
| | | | | | | | | | | | | | | | | CAAATGACCAGCCTGAGAGCTGAGGAC | | TAVYYCVRDP | | | |
| | | | | | | | | | | | | | | | | ACGGCTGTATATTACTGTGTGAGAGAT | | QDCGGSRCSN | | | |
| | | | | | | | | | | | | | | | | CCCCAAGATTGTGGTGGTTCCAGGTGT | | ALGYFDYWGQ | | | |
| | | | | | | | | | | | | | | | | TCAAACGCGCTTGGTTACTTTGACTAC | | GTLVTVSS | | | |
| | | | | | | | | | | | | | | | | TGGGGCCAGGGAACCCTGGTCACCGTC | | | | | |
| | | | | | | | | | | | | | | | | TCCTCA | | | | | |
| ADI- | IGHV3- | QVQLVQSG | 10 | ITFRSNG | 276 | WVRQA | 431 | FIWFDG | 603 | RFIIS | 859 | AREPHC | 1103 | WGQGTT | 1287 | CAGGTGCAGCTGGTGCAGTCTGGGGGA | 1393 | QVQLVQSGGG | 1676 | | |
| 75861 | 33 | GGVVQPGR | | MH | | PGKGL | | SKKYYG | | RDNSN | | TGGVCD | | VTVSS | | GGCGTGGTCCAGCCTGGGAGGTCCCTG | | VVQPGRSLRL | | | |
| | | SLRLSCAA | | | | EWVA | | DSVKG | | NTLYL | | ALDI | | | | AGACTCTCCTGTGCAGCGTCTGGAATC | | SCAASGITFR | | | |
| | | SG | | | | | | | | QMNSL | | | | | | ACCTTCAGGAGCAATGGCATGCACTGG | | SNGMHWVRQA | | | |
| | | | | | | | | | | RAEDT | | | | | | GTCCGCCAGGCTCCAGGCAAGGGGCTG | | PGKGLEWVAF | | | |
| | | | | | | | | | | AVYYC | | | | | | GAGTGGGTGGCGTTTATATGGTTTGAT | | IWFDGSKKYY | | | |
| | | | | | | | | | | | | | | | | GGAAGTAAGAAATATTATGGAGACTCC | | GDSVKGRFII | | | |
| | | | | | | | | | | | | | | | | GTGAAGGGCCGATTCATCATCTCCAGA | | SRDNSNNTLY | | | |
| | | | | | | | | | | | | | | | | GACAATTCCAACAACACGCTGTATCTG | | LQMNSLRAED | | | |
| | | | | | | | | | | | | | | | | CAAATGAACAGCCTGAGAGCCGAGGAC | | TAVYYCAREP | | | |
| | | | | | | | | | | | | | | | | ACGGCTGTCTATTACTGTGCGAGAGAA | | HCTGGVCDAL | | | |
| | | | | | | | | | | | | | | | | CCCCACTGCACTGGTGGTGTATGCGAT | | DIWGQGTTVT | | | |
| | | | | | | | | | | | | | | | | GCTCTTGATATCTGGGGCCAAGGGACC | | VSS | | | |
| | | | | | | | | | | | | | | | | ACGGTCACCGTCTCCTCA | | | | | |
| ADI- | IGHV3- | QVQLVQSG | 76 | FIFSNYD | 277 | WVRQT | 464 | AIGTAG | 604 | RFTIS | 860 | ARAEHS | 1104 | WGRGTL | 1288 | CAGGTGCAGCTGGTGCAGTCTGGGGGA | 1394 | QVQLVQSGGG | 1677 | | |
| 75578 | 13 | GGLVQPGG | | MH | | TGKGL | | DTYYAD | | RENAK | | GFVVYF | | VTVSS | | GGCCTGGTGCAGCCGGGGGGGTCCCTG | | LVQPGGSLRL | | | |
| | | SLRLSCGA | | | | EWVS | | SVKG | | NSLYL | | DL | | | | AGACTCTCCTGTGGAGCCTCTGGATTC | | SCGASGFIFS | | | |
| | | SG | | | | | | | | QMNSL | | | | | | ATCTTCAGTAACTACGACATGCACTGG | | NYDMHWVRQT | | | |
| | | | | | | | | | | RAGDT | | | | | | GTCCGCCAAACTACAGGAAAAGGTCTG | | TGKGLEWVSA | | | |
| | | | | | | | | | | AVYYC | | | | | | GAGTGGGTCTCAGCTATTGGAACTGCT | | IGTAGDTYYA | | | |
| | | | | | | | | | | | | | | | | GGTGACACATACTATGCAGACTCCGTG | | DSVKGRFTIS | | | |
| | | | | | | | | | | | | | | | | AAGGGCCGTTTCACCATCTCCAGAGAA | | RENAKNSLYL | | | |
| | | | | | | | | | | | | | | | | AATGCCAAGAACTCCTTGTATCTTCAA | | QMNSLRAGDT | | | |
| | | | | | | | | | | | | | | | | ATGAACAGCCTGAGAGCCGGGGACACG | | AVYYCARAEH | | | |
| | | | | | | | | | | | | | | | | GCTGTGTATTACTGTGCAAGAGCTGAA | | SGFVVYFDLW | | | |
| | | | | | | | | | | | | | | | | CATAGTGGCTTCGTTGTCTACTTCGAT | | GRGTLVTVSS | | | |
| | | | | | | | | | | | | | | | | CTCTGGGGCCGTGGCACCCTGGTCACC | | | | | |
| | | | | | | | | | | | | | | | | GTCTCCTCA | | | | | |
| ADI- | IGHV2- | EVQLVQSG | 77 | FSFSTSG | 278 | WIRQP | 441 | LIYWDG | 605 | RLTIA | 861 | ARHTVA | 1105 | WGQGTL | 1286 | GAGGTGCAGCTGGTGCAGTCTGGTCCT | 1395 | EVQLVQSGPT | 1678 | | |
| 75808 | 5 | PTLVKPTQ | | MGVG | | PGKAL | | DERYSP | | RDTSK | | TIFDY | | VTVSS | | ACGCTGGTGAAACCCACACAGACCGTC | | LVKPTQTVTL | | | |
| | | TVTLTCTF | | | | EWLA | | SLQS | | NQVVL | | | | | | ACGCTGACCTGCACCTTCTCTGGGTTC | | TCTFSGFSFS | | | |
| | | SG | | | | | | | | TMTNM | | | | | | TCGTTCAGTACTAGTGGAATGGGTGTG | | TSGMGVGWIR | | | |
| | | | | | | | | | | DPVDT | | | | | | GGCTGGATCCGTCAGCCCCCAGGAAAG | | QPPGKALEWL | | | |
| | | | | | | | | | | ATYYC | | | | | | GCCCTGGAGTGGCTTGCACTCATTTAT | | ALIYWDGDER | | | |
| | | | | | | | | | | | | | | | | TGGGATGGTGATGAGCGCTACAGCCCA | | YSPSLQSRLT | | | |
| | | | | | | | | | | | | | | | | TCTCTGCAGAGCAGGCTCACCATCGCC | | IARDTSKNQV | | | |
| | | | | | | | | | | | | | | | | AGGGACACCTCCAAAAACCAGGTGGTC | | VLTMTNMDPV | | | |
| | | | | | | | | | | | | | | | | CTTACAATGACCAACATGGACCCTGTG | | DTATYYCARH | | | |
| | | | | | | | | | | | | | | | | GACACAGCCACATATTACTGTGCACGC | | TVATIFDYWG | | | |
| | | | | | | | | | | | | | | | | CATACAGTGGCGACCATTTTTGACTAC | | QGTLVTVSS | | | |
| | | | | | | | | | | | | | | | | TGGGGCCAGGGAACCCTGGTCACCGTC | | | | | |
| | | | | | | | | | | | | | | | | TCCTCA | | | | | |
| ADI- | IGHV3- | QVQLVESG | 78 | FIFTTYW | 279 | WVRQA | 431 | NIKQDG | 606 | RFTIS | 862 | ARELGI | 1106 | WGQGTL | 1286 | CAGGTGCAGCTGGTGGAGTCTGGGGGA | 1396 | QVQLVESGGG | 1679 | | |
| 75848 | 7 | GGLVQPGG | | MS | | PGKGL | | SEKYYV | | RDNAK | | SDY | | VTVSS | | GGCTTGGTCCAGCCTGGGGGGTCCCTG | | LVQPGGSLRL | | | |
| | | SLRLSCEA | | | | EWVA | | DSVKG | | NSLYL | | | | | | AGACTCTCCTGTGAAGCCTCTGGATTC | | SCEASGFIFT | | | |
| | | SG | | | | | | | | QMDSL | | | | | | ATCTTTACAACCTATTGGATGAGTTGG | | TYWMSWVRQA | | | |
| | | | | | | | | | | RAEDT | | | | | | GTCCGCCAGGCTCCAGGGAAGGGGCTG | | PGKGLEWVAN | | | |
| | | | | | | | | | | AVYYC | | | | | | GAGTGGGTGGCCAACATAAAGCAAGAT | | IKQDGSEKYY | | | |
| | | | | | | | | | | | | | | | | GGAAGTGAGAAATACTATGTGGACTCT | | VDSVKGRFTI | | | |
| | | | | | | | | | | | | | | | | GTGAAGGGCCGATTCACCATCTCCAGA | | SRDNAKNSLY | | | |
| | | | | | | | | | | | | | | | | GACAACGCCAAGAACTCACTGTATCTG | | LQMDSLRAED | | | |
| | | | | | | | | | | | | | | | | CAAATGGACAGCCTGAGAGCCGAGGAC | | TAVYYCAREL | | | |
| | | | | | | | | | | | | | | | | ACGGCCGTTTATTACTGTGCGAGAGAG | | GISDYWGQGT | | | |
| | | | | | | | | | | | | | | | | CTGGGGATCTCGGACTACTGGGGCCAG | | LVTVSS | | | |
| | | | | | | | | | | | | | | | | GGAACCCTGGTCACCGTCTCCTCA | | | | | |
| ADI- | IGHV3- | QVQLVQSG | 79 | FIVSANY | 280 | WVRQA | 465 | VLYSGG | 607 | RFTIS | 863 | VRDWGE | 1107 | WGQGTL | 1286 | CAGGTGCAGCTGGTGCAGTCTGGAGGA | 1397 | QVQLVQSGGG | 1680 | | |
| 75832 | 53 | GGLIQPGG | | MS | | PGKRL | | TTFYAD | | RDNSK | | FFFDF | | VTVSS | | GGCTTGATCCAGCCGGGGGGGTCCCTG | | LIQPGGSLRL | | | |
| | | SLRLSCAA | | | | EWVS | | SVKG | | NTLYL | | | | | | AGACTCTCCTGTGCAGCCTCTGGGTTC | | SCAASGFIVS | | | |
| | | SG | | | | | | | | QMNSL | | | | | | ATCGTCAGTGCCAACTACATGAGCTGG | | ANYMSWVRQA | | | |
| | | | | | | | | | | RADDT | | | | | | GTCCGCCAGGCTCCAGGGAAGCGGCTG | | PGKRLEWVSV | | | |
| | | | | | | | | | | AVYFC | | | | | | GAGTGGGTCTCAGTTCTTTATAGCGGC | | LYSGGTTFYA | | | |
| | | | | | | | | | | | | | | | | GGTACCACATTCTACGCCGACTCCGTG | | DSVKGRFTIS | | | |
| | | | | | | | | | | | | | | | | AAGGGCCGATTCACCATCTCCAGAGAC | | RDNSKNTLYL | | | |
| | | | | | | | | | | | | | | | | AATTCCAAGAACACGCTGTATCTTCAA | | QMNSLRADDT | | | |
| | | | | | | | | | | | | | | | | ATGAACAGCCTGAGAGCCGACGACACG | | AVYFCVRDWG | | | |
| | | | | | | | | | | | | | | | | GCCGTGTATTTTTGTGTGAGAGATTGG | | EFFFDFWGQG | | | |
| | | | | | | | | | | | | | | | | GGGGAGTTCTTCTTTGACTTCTGGGGC | | TLVTVSS | | | |
| | | | | | | | | | | | | | | | | CAGGGTACCCTGGTCACCGTCTCCTCA | | | | | |
| ADI- | IGHV3- | EVQLVETG | 80 | FTVSSNY | 281 | WVRQA | 437 | IIYPGG | 608 | RFTIS | 864 | ARDRGG | 1108 | WGQGTL | 1286 | GAGGTGCAGCTGGTGGAGACTGGAGGA | 1398 | EVQLVETGGG | 1681 | | |
| 75618 | 53 | GGLIQPGG | | MS | | PGKGL | | SSFYAE | | RDSSK | | YPDY | | VTVSS | | GGCTTGATCCAGCCTGGGGGGTCCCTG | | LIQPGGSLRL | | | |
| | | SLRLSCAA | | | | EWVS | | SVKG | | NTLYL | | | | | | AGACTCTCCTGTGCAGCCTCTGAGTTC | | SCAASEFTVS | | | |
| | | SE | | | | | | | | QMDSL | | | | | | ACCGTCAGTAGCAACTACATGAGCTGG | | SNYMSWVRQA | | | |
| | | | | | | | | | | RAEDT | | | | | | GTCCGCCAGGCTCCAGGGAAGGGACTG | | PGKGLEWVSI | | | |
| | | | | | | | | | | AVYYC | | | | | | GAGTGGGTCTCAATTATTTATCCCGGT | | IYPGGSSFYA | | | |
| | | | | | | | | | | | | | | | | GGTAGCTCATTCTACGCAGAGTCCGTG | | ESVKGRFTIS | | | |
| | | | | | | | | | | | | | | | | AAGGGCCGATTCACCATCTCCAGAGAC | | RDSSKNTLYL | | | |
| | | | | | | | | | | | | | | | | AGTTCCAAGAACACGCTCTATCTTCAA | | QMDSLRAEDT | | | |
| | | | | | | | | | | | | | | | | ATGGACAGCCTGAGAGCCGAGGACACG | | AVYYCARDRG | | | |
| | | | | | | | | | | | | | | | | GCCGTGTATTACTGTGCGAGAGATCGG | | GYPDYWGQGT | | | |
| | | | | | | | | | | | | | | | | GGTGGGTACCCTGACTACTGGGGCCAG | | LVTVSS | | | |
| | | | | | | | | | | | | | | | | GGAACCCTGGTCACCGTCTCCTCA | | | | | |
| ADI- | IGHV3- | QVQLVQSG | 10 | FTFSSFP | 282 | WIRQA | 466 | VVSYDG | 609 | RFTIS | 865 | AREMHY | 1109 | WGQGTL | 1286 | CAGGTCCAGCTGGTGCAGTCTGGGGGA | 1399 | QVQLVQSGGG | 1682 | | |
| 75830 | 30 | GGVVQPGR | | MH | | PGKGL | | SSKHYT | | RDNSM | | YDSSGY | | VTVSS | | GGCGTGGTCCAGCCTGGGAGGTCCCTG | | VVQPGRSLRL | | | |
| | | SLRLSCAA | | | | QWVA | | DSVKG | | NIVYL | | FKGNNY | | | | AGACTCTCCTGTGCAGCCTCTGGATTC | | SCAASGFTFS | | | |
| | | SG | | | | | | | | QMNSL | | FDP | | | | ACCTTCAGTAGTTTTCCTATGCACTGG | | SFPMHWIRQA | | | |
| | | | | | | | | | | RAEDT | | | | | | ATCCGCCAGGCTCCAGGCAAGGGGCTA | | PGKGLQWVAV | | | |
| | | | | | | | | | | AVYYC | | | | | | CAGTGGGTGGCAGTTGTGTCATATGAT | | VSYDGSSKHY | | | |
| | | | | | | | | | | | | | | | | GGAAGTAGTAAACACTATACAGACTCC | | TDSVKGRFTI | | | |
| | | | | | | | | | | | | | | | | GTGAAGGGCCGATTCACCATCTCCAGA | | SRDNSMNIVY | | | |
| | | | | | | | | | | | | | | | | GACAACTCCATGAACATCGTGTATCTG | | LQMNSLRAED | | | |
| | | | | | | | | | | | | | | | | CAAATGAACAGCCTGAGAGCTGAGGAC | | TAVYYCAREM | | | |
| | | | | | | | | | | | | | | | | ACGGCTGTGTATTACTGTGCGAGAGAA | | HYYDSSGYFK | | | |
| | | | | | | | | | | | | | | | | ATGCATTACTATGATAGTAGTGGTTAT | | GNNYFDPWGQ | | | |
| | | | | | | | | | | | | | | | | TTTAAAGGCAATAACTACTTCGACCCC | | GTLVTVSS | | | |
| | | | | | | | | | | | | | | | | TGGGGCCAGGGAACCCTGGTCACCGTC | | | | | |
| | | | | | | | | | | | | | | | | TCCTCA | | | | | |
| ADI- | IGHV3- | EVQLVETG | 81 | IIVSYNY | 283 | WVRQA | 437 | LIYAGG | 610 | RFTIS | 777 | TRDRGP | 1110 | WGQGTL | 1286 | GAGGTGCAGCTGGTGGAGACTGGAGGA | 1400 | EVQLVETGGG | 1683 | | |
| 75628 | 53 | GGLIQPGG | | MS | | PGKGL | | STYYAD | | RDNSK | | LLHDL | | VTVSS | | GGATTGATCCAGCCGGGGGGGTCCCTG | | LIQPGGSLRL | | | |
| | | SLRLSCAA | | | | EWVS | | SVKG | | NTLYL | | | | | | AGACTCTCCTGTGCAGCCTCTGGGATC | | SCAASGIIVS | | | |
| | | SG | | | | | | | | QMNSL | | | | | | ATCGTCAGTTACAACTACATGAGTTGG | | YNYMSWVRQA | | | |
| | | | | | | | | | | RAEDT | | | | | | GTCCGCCAGGCTCCAGGGAAGGGGCTG | | PGKGLEWVSL | | | |
| | | | | | | | | | | AVYYC | | | | | | GAGTGGGTCTCACTCATTTATGCCGGT | | IYAGGSTYYA | | | |
| | | | | | | | | | | | | | | | | GGTAGCACCTACTACGCAGACTCCGTG | | DSVKGRFTIS | | | |
| | | | | | | | | | | | | | | | | AAGGGCCGTTTCACCATCTCCAGAGAC | | RDNSKNTLYL | | | |
| | | | | | | | | | | | | | | | | AATTCCAAGAACACACTATATCTTCAA | | QMNSLRAEDT | | | |
| | | | | | | | | | | | | | | | | ATGAACAGCCTGAGAGCCGAGGACACG | | AVYYCTRDRG | | | |
| | | | | | | | | | | | | | | | | GCCGTGTATTACTGTACGAGAGATAGG | | PLLHDLWGQG | | | |
| | | | | | | | | | | | | | | | | GGGCCTTTACTACACGACCTCTGGGGC | | TLVTVSS | | | |
| | | | | | | | | | | | | | | | | CAGGGAACCCTGGTCACCGTCTCCTCA | | | | | |
| ADI- | IGHV4- | QVQLQESG | 39 | GSISSGN | 284 | WIRQS | 434 | YIYYSG | 611 | RVTIS | 866 | VRERIS | 1111 | WGQGTT | 1287 | CAGGTGCAGCTGCAGGAGTCTGGCCCA | 1401 | QVQLQESGPG | 1684 | | |
| 75640 | 30-1 | PGLVKPSQ | | YYWS | | PGKGL | | STYYNP | | VDTSK | | MDILSG | | VTVSS | | GGACTGGTGAAGCCTTCACAGACCCTG | | LVKPSQTLSL | | | |
| | | TLSLTCTV | | | | EWIG | | SLKS | | DQFSL | | NSPHYY | | | | TCCCTCACCTGCACTGTCTCTGGTGGC | | TCTVSGGSIS | | | |
| | | SG | | | | | | | | KLSSV | | GVDV | | | | TCCATCAGCAGTGGTAATTACTACTGG | | SGNYYWSWIR | | | |
| | | | | | | | | | | TAADT | | | | | | AGTTGGATCCGCCAGTCCCCAGGGAAG | | QSPGKGLEWI | | | |
| | | | | | | | | | | AVYHC | | | | | | GGCCTGGAGTGGATTGGGTACATCTAT | | GYIYYSGSTY | | | |
| | | | | | | | | | | | | | | | | TACAGTGGGAGCACCTACTACAATCCG | | YNPSLKSRVT | | | |
| | | | | | | | | | | | | | | | | TCCCTCAAGAGTCGAGTTACCATATCA | | ISVDTSKDQF | | | |
| | | | | | | | | | | | | | | | | GTAGACACGTCCAAGGACCAGTTCTCC | | SLKLSSVTAA | | | |
| | | | | | | | | | | | | | | | | CTGAAGCTGAGCTCTGTGACTGCCGCA | | DTAVYHCVRE | | | |
| | | | | | | | | | | | | | | | | GACACGGCCGTGTATCACTGTGTCAGA | | RISMDILSGN | | | |
| | | | | | | | | | | | | | | | | GAAAGGATTTCTATGGATATTTTGAGT | | SPHYYGVDVW | | | |
| | | | | | | | | | | | | | | | | GGTAATTCCCCCCACTACTACGGAGTG | | GQGTTVTVSS | | | |
| | | | | | | | | | | | | | | | | GACGTCTGGGGCCAAGGGACCACGGTC | | | | | |
| | | | | | | | | | | | | | | | | ACCGTCTCCTCA | | | | | |
| ADI- | IGHV3- | QVQLVQSG | 82 | FTFEDYA | 264 | WVRLL | 458 | GVSWNS | 589 | RFIIS | 845 | VKLYSG | 1112 | WGQGTT | 1287 | CAGGTGCAGCTGGTGCAGTCTGGGGGA | 1402 | QVQLVQSGGG | 1685 | | |
| 75657 | 9 | GGLVQPGR | | IH | | PGKGL | | GVIDYV | | RDNAK | | GRDYDF | | VTVSS | | GGCTTGGTGCAGCCTGGCAGGTCCCTG | | LVQPGRSLTL | | | |
| | | SLTLSCTA | | | | EWVS | | DSVKG | | NSLYL | | RSGSQN | | | | ACACTCTCCTGTACAGCCTCTGGATTC | | SCTASGFTFE | | | |
| | | SG | | | | | | | | HMRSL | | GGGDAM | | | | ACGTTTGAAGATTATGCCATACACTGG | | DYAIHWVRLL | | | |
| | | | | | | | | | | RPEDT | | DV | | | | GTCCGGCTACTTCCAGGGAAGGGCCTG | | PGKGLEWVSG | | | |
| | | | | | | | | | | ALYYC | | | | | | GAGTGGGTCTCGGGTGTTAGTTGGAAT | | VSWNSGVIDY | | | |
| | | | | | | | | | | | | | | | | AGTGGTGTCATAGACTACGTGGACTCT | | VDSVKGRFII | | | |
| | | | | | | | | | | | | | | | | GTGAAGGGCCGATTCATCATCTCCAGA | | SRDNAKNSLY | | | |
| | | | | | | | | | | | | | | | | GACAACGCCAAGAACTCCTTGTATCTG | | LHMRSLRPED | | | |
| | | | | | | | | | | | | | | | | CATATGAGAAGTCTGAGACCTGAGGAC | | TALYYCVKLY | | | |
| | | | | | | | | | | | | | | | | ACGGCCTTATATTACTGTGTAAAATTA | | SGGRDYDFRS | | | |
| | | | | | | | | | | | | | | | | TACTCGGGGGGGAGGGACTACGATTTT | | GSQNGGGDAM | | | |
| | | | | | | | | | | | | | | | | CGGAGTGGTTCGCAAAATGGGGGTGGA | | DVWGQGTTVT | | | |
| | | | | | | | | | | | | | | | | GACGCTATGGACGTCTGGGGCCAAGGG | | VSS | | | |
| | | | | | | | | | | | | | | | | ACCACGGTCACCGTCTCCTCA | | | | | |
| ADI- | IGHV2- | RSTLQQSG | 83 | FSLATTG | 285 | WIRQP | 441 | LIYWDD | 612 | RVTIT | 867 | AHMVGR | 1113 | WGKGTT | 1291 | AGGTCCACGCTGCAGCAGTCTGGTCCT | 1403 | RSTLQQSGPT | 1686 | | |
| 75713 | 5 | PTLVKPTQ | | VGVG | | PGKAL | | DKRYSP | | KDTFK | | SYYDVL | | VTVSS | | ACGCTGGTGAAACCCACACAGACCCTC | | LVKPTQTLTL | | | |
| | | TLTLTCTF | | | | EWLA | | SLKS | | NQVVL | | TGFSSY | | | | ACGCTGACCTGCACCTTCTCTGGGTTC | | TCTFSGFSLA | | | |
| | | SG | | | | | | | | TMTNM | | YSYYMD | | | | TCACTCGCAACTACTGGAGTGGGTGTG | | TTGVGVGWIR | | | |
| | | | | | | | | | | DPVDT | | V | | | | GGCTGGATCCGTCAGCCCCCAGGAAAG | | QPPGKALEWL | | | |
| | | | | | | | | | | GTYYC | | | | | | GCCCTGGAATGGCTTGCACTCATTTAT | | ALIYWDDDKR | | | |
| | | | | | | | | | | | | | | | | TGGGATGATGATAAGCGCTACAGCCCA | | YSPSLKSRVT | | | |
| | | | | | | | | | | | | | | | | TCTCTGAAGAGCAGGGTCACCATCACC | | ITKDTFKNQV | | | |
| | | | | | | | | | | | | | | | | AAGGACACCTTCAAAAACCAGGTGGTC | | VLTMTNMDPV | | | |
| | | | | | | | | | | | | | | | | CTTACAATGACCAACATGGACCCGGTG | | DTGTYYCAHM | | | |
| | | | | | | | | | | | | | | | | GACACAGGCACATATTACTGTGCACAC | | VGRSYYDVLT | | | |
| | | | | | | | | | | | | | | | | ATGGTTGGTCGTTCGTATTACGATGTT | | GFSSYYSYYM | | | |
| | | | | | | | | | | | | | | | | TTGACTGGTTTTTCTTCCTACTACTCC | | DVWGKGTTVT | | | |
| | | | | | | | | | | | | | | | | TACTACATGGACGTTTGGGGCAAAGGG | | VSS | | | |
| | | | | | | | | | | | | | | | | ACCACGGTCACCGTCTCCTCA | | | | | |
| ADI- | IGHV2- | QVTLMESG | 84 | FSLSTSG | 286 | WIRQP | 441 | LIYWDD | 613 | RLTVT | 868 | AHRPRS | 1114 | WGQGTL | 1286 | CAGGTCACGCTGATGGAGTCTGGTCGT | 1404 | QVTLMESGRT | 1687 | | |
| 75692 | 5 | RTLVKPTQ | | VSVG | | PGKAL | | DTRYSP | | KDTSK | | QSGYDS | | VTVSS | | ACGCTGGTGAAACCCACACAGACCCTC | | LVKPTQTLTL | | | |
| | | TLTLTCTF | | | | EWLA | | SLKR | | NQVVL | | SFDV | | | | ACGCTGACCTGCACCTTCTCTGGGTTC | | TCTFSGFSLS | | | |
| | | SG | | | | | | | | TLTNL | | | | | | TCACTCAGTACTAGTGGAGTGAGTGTG | | TSGVSVGWIR | | | |
| | | | | | | | | | | GAVDT | | | | | | GGCTGGATCCGTCAGCCCCCAGGAAAG | | QPPGKALEWL | | | |
| | | | | | | | | | | GTYYC | | | | | | GCCCTGGAGTGGCTTGCACTCATTTAT | | ALIYWDDDTR | | | |
| | | | | | | | | | | | | | | | | TGGGATGATGATACGCGCTACAGCCCA | | YSPSLKRRLT | | | |
| | | | | | | | | | | | | | | | | TCTCTGAAGAGGAGGCTCACCGTCACC | | VTKDTSKNQV | | | |
| | | | | | | | | | | | | | | | | AAGGACACCTCCAAAAACCAGGTGGTC | | VLTLTNLGAV | | | |
| | | | | | | | | | | | | | | | | CTTACACTGACCAACTTGGGCGCTGTG | | DTGTYYCAHR | | | |
| | | | | | | | | | | | | | | | | GACACAGGCACATATTACTGTGCACAC | | PRSQSGYDSS | | | |
| | | | | | | | | | | | | | | | | AGACCGAGGTCACAAAGTGGCTACGAC | | FDVWGQGTLV | | | |
| | | | | | | | | | | | | | | | | TCGTCCTTTGACGTCTGGGGCCAGGGA | | TVSS | | | |
| | | | | | | | | | | | | | | | | ACCCTGGTCACCGTCTCCTCA | | | | | |
| ADI- | IGHV3- | QVQLVQSG | 12 | FTFSSYD | 207 | WVRQA | 437 | GIGTAG | 614 | RFTIS | 869 | ARVEYS | 1115 | WGRGTL | 1288 | CAGGTGCAGCTGGTGCAGTCTGGGGGA | 1405 | QVQLVQSGGG | 1688 | | |
| 75587 | 13 | GGLVQPGG | | MH | | PGKGL | | DTYYAD | | RENAK | | SGWSYW | | VTVSS | | GGCTTGGTACAGCCTGGGGGGTCCCTG | | LVQPGGSLRL | | | |
| | | SLRLSCAA | | | | EWVS | | SVKG | | NSFYL | | FFDL | | | | AGACTCTCCTGTGCAGCCTCCGGATTC | | SCAASGFTFS | | | |
| | | SG | | | | | | | | QMNSL | | | | | | ACCTTCAGTAGCTACGACATGCACTGG | | SYDMHWVRQA | | | |
| | | | | | | | | | | RAGDT | | | | | | GTCCGCCAGGCTCCAGGAAAAGGTCTG | | PGKGLEWVSG | | | |
| | | | | | | | | | | AVYYC | | | | | | GAATGGGTCTCAGGTATTGGTACTGCT | | IGTAGDTYYA | | | |
| | | | | | | | | | | | | | | | | GGTGACACATACTATGCAGACTCCGTG | | DSVKGRFTIS | | | |
| | | | | | | | | | | | | | | | | AAGGGCCGATTCACCATCTCCAGAGAA | | RENAKNSFYL | | | |
| | | | | | | | | | | | | | | | | AATGCCAAGAACTCCTTCTATCTTCAA | | QMNSLRAGDT | | | |
| | | | | | | | | | | | | | | | | ATGAACAGCCTGAGAGCCGGGGACACG | | AVYYCARVEY | | | |
| | | | | | | | | | | | | | | | | GCTGTGTATTACTGTGCAAGAGTGGAG | | SSGWSYWFFD | | | |
| | | | | | | | | | | | | | | | | TATAGTAGCGGCTGGTCGTACTGGTTC | | LWGRGTLVTV | | | |
| | | | | | | | | | | | | | | | | TTCGATCTCTGGGGCCGTGGCACCCTG | | SS | | | |
| | | | | | | | | | | | | | | | | GTCACCGTCTCCTCA | | | | | |
| ADI- | IGHV3- | EVQLVQSG | 85 | FTLSSDD | 287 | WVRQA | 467 | GIGTSG | 615 | RFSIS | 870 | ARGRST | 1116 | WGKGTT | 1291 | GAGGTGCAGCTGGTGCAGTCTGGGGGA | 1406 | EVQLVQSGGG | 1689 | | |
| 75775 | 13 | GGLAQPGG | | MH | | TGKGL | | DTYYPD | | RENAK | | MIQGDK | | VTVSS | | GGCTTGGCACAGCCTGGGGGGTCCCTG | | LAQPGGSLRL | | | |
| | | SLRLSCAA | | | | EWVS | | SVKG | | NSLYL | | KYYHYY | | | | AGACTCTCCTGTGCAGCCTCTGGATTC | | SCAASGFTLS | | | |
| | | SG | | | | | | | | QMNSL | | YMDV | | | | ACCCTCAGTAGCGACGACATGCACTGG | | SDDMHWVRQA | | | |
| | | | | | | | | | | RAGDT | | | | | | GTCCGCCAAGCTACAGGAAAAGGTCTG | | TGKGLEWVSG | | | |
| | | | | | | | | | | AVYYC | | | | | | GAGTGGGTCTCAGGTATTGGTACGTCT | | IGTSGDTYYP | | | |
| | | | | | | | | | | | | | | | | GGTGACACATACTATCCAGACTCCGTG | | DSVKGRFSIS | | | |
| | | | | | | | | | | | | | | | | AAGGGCCGATTCAGCATCTCCAGAGAA | | RENAKNSLYL | | | |
| | | | | | | | | | | | | | | | | AATGCCAAGAACTCACTGTATCTTCAA | | QMNSLRAGDT | | | |
| | | | | | | | | | | | | | | | | ATGAACAGCCTGAGAGCCGGGGACACG | | AVYYCARGRS | | | |
| | | | | | | | | | | | | | | | | GCTGTGTATTACTGTGCAAGAGGACGT | | TMIQGDKKYY | | | |
| | | | | | | | | | | | | | | | | TCTACTATGATTCAGGGAGACAAAAAA | | HYYYMDVWGK | | | |
| | | | | | | | | | | | | | | | | TACTACCACTACTACTACATGGACGTC | | GTTVTVSS | | | |
| | | | | | | | | | | | | | | | | TGGGGCAAAGGGACCACGGTCACCGTC | | | | | |
| | | | | | | | | | | | | | | | | TCCTCA | | | | | |
| ADI- | IGHV1- | QVQLQQSG | 86 | GTFISDA | 288 | WVRQA | 433 | RIIPIL | 616 | RVTLT | 871 | AIDRDY | 1117 | WGKGTT | 1291 | CAGGTGCAGCTGCAGCAGTCTGGGGCT | 1407 | QVQLQQSGAE | 1690 | | |
| 75758 | 69 | AEVKKPGS | | VS | | PGQGL | | GTATYA | | ADKST | | PGDYYY | | VTVSS | | GAGGTGAAGAAGCCTGGGTCCTCGGTG | | VKKPGSSVKV | | | |
| | | SVKVSCKA | | | | EWMG | | QKFQG | | STAYM | | HYYTDV | | | | AAGGTCTCCTGCAAGGCTTCTGGAGGC | | SCKASGGTFI | | | |
| | | SG | | | | | | | | ELRSL | | | | | | ACCTTCATCAGCGATGCTGTCAGCTGG | | SDAVSWVRQA | | | |
| | | | | | | | | | | RSDDT | | | | | | GTGCGACAGGCCCCTGGACAAGGGCTT | | PGQGLEWMGR | | | |
| | | | | | | | | | | AVYYC | | | | | | GAGTGGATGGGAAGGATCATCCCTATA | | IIPILGTATY | | | |
| | | | | | | | | | | | | | | | | CTGGGAACAGCTACCTACGCACAGAAG | | AQKFQGRVTL | | | |
| | | | | | | | | | | | | | | | | TTCCAGGGCAGAGTCACGCTTACCGCG | | TADKSTSTAY | | | |
| | | | | | | | | | | | | | | | | GACAAATCCACGAGCACAGCCTACATG | | MELRSLRSDD | | | |
| | | | | | | | | | | | | | | | | GAACTGCGCAGCCTGAGATCTGACGAC | | TAVYYCAIDR | | | |
| | | | | | | | | | | | | | | | | ACGGCCGTCTATTACTGTGCGATAGAC | | DYPGDYYYHY | | | |
| | | | | | | | | | | | | | | | | AGGGACTACCCGGGCGATTACTACTAT | | YTDVWGKGTT | | | |
| | | | | | | | | | | | | | | | | CACTACTACACGGACGTCTGGGGCAAA | | VTVSS | | | |
| | | | | | | | | | | | | | | | | GGGACCACGGTCACCGTCTCCTCA | | | | | |
| ADI- | IGHV3- | QVQLVQSG | 87 | ITVSSNY | 289 | WVRQA | 437 | VIYAGG | 617 | RFSIS | 872 | ARDLGP | 1118 | WGQGTT | 1287 | CAGGTGCAGCTGGTGCAGTCTGGGGGA | 1408 | QVQLVQSGGG | 1691 | | |
| 75793 | 66 | GGLVRPGG | | MN | | PGKGL | | STFYAD | | RDNSK | | YGFDI | | VTVSS | | GGCTTGGTCCGGCCGGGGGGGTCTCTG | | LVRPGGSLRL | | | |
| | | SLRLSCAA | | | | EWVS | | SVKG | | NTLYL | | | | | | AGACTCTCCTGTGCAGCCTCTGAAATC | | SCAASEITVS | | | |
| | | SE | | | | | | | | QMNSL | | | | | | ACCGTCAGTAGTAACTACATGAACTGG | | SNYMNWVRQA | | | |
| | | | | | | | | | | RADDT | | | | | | GTCCGCCAGGCTCCAGGGAAGGGGCTG | | PGKGLEWVSV | | | |
| | | | | | | | | | | AVYYC | | | | | | GAGTGGGTCTCAGTTATATATGCCGGT | | IYAGGSTFYA | | | |
| | | | | | | | | | | | | | | | | GGTAGCACATTCTACGCAGACTCCGTG | | DSVKGRFSIS | | | |
| | | | | | | | | | | | | | | | | AAGGGCAGATTCAGCATCTCCAGAGAC | | RDNSKNTLYL | | | |
| | | | | | | | | | | | | | | | | AATTCCAAAAACACGCTGTATCTTCAA | | QMNSLRADDT | | | |
| | | | | | | | | | | | | | | | | ATGAACAGCCTGAGAGCCGACGACACG | | AVYYCARDLG | | | |
| | | | | | | | | | | | | | | | | GCTGTGTATTACTGTGCGAGAGACTTG | | PYGFDIWGQG | | | |
| | | | | | | | | | | | | | | | | GGCCCCTACGGATTTGATATCTGGGGC | | TTVTVSS | | | |
| | | | | | | | | | | | | | | | | CAAGGGACCACGGTCACCGTCTCCTCA | | | | | |
| ADI- | IGHV3- | QVQLVQSG | 12 | FTFSNYD | 290 | WVRQV | 468 | TIGTAG | 618 | RFTIS | 873 | ARVQYS | 1119 | WGKGTT | 1291 | CAGGTGCAGCTGGTGCAGTCTGGGGGA | 1409 | QVQLVQSGGG | 1692 | | |
| 75804 | 13 | GGLVQPGG | | MH | | TGKGL | | DTYYPD | | RDNAK | | SGRHQY | | VTVSS | | GGCTTGGTACAGCCTGGGGGGTCCCTG | | LVQPGGSLRL | | | |
| | | SLRLSCAA | | | | EWVS | | SVKG | | NSLYL | | SYYYMD | | | | AGACTCTCCTGTGCAGCCTCTGGATTC | | SCAASGFTFS | | | |
| | | SG | | | | | | | | QMNSL | | V | | | | ACCTTCAGTAACTACGACATGCACTGG | | NYDMHWVRQV | | | |
| | | | | | | | | | | RAGDT | | | | | | GTCCGCCAAGTCACAGGAAAAGGTCTG | | TGKGLEWVST | | | |
| | | | | | | | | | | AVYFC | | | | | | GAGTGGGTTTCAACTATTGGTACTGCT | | IGTAGDTYYP | | | |
| | | | | | | | | | | | | | | | | GGTGACACATACTATCCAGACTCCGTG | | DSVKGRFTIS | | | |
| | | | | | | | | | | | | | | | | AAGGGCCGATTCACCATCTCCAGAGAC | | RDNAKNSLYL | | | |
| | | | | | | | | | | | | | | | | AATGCCAAGAACTCCTTGTATCTTCAA | | QMNSLRAGDT | | | |
| | | | | | | | | | | | | | | | | ATGAACAGCCTGAGAGCCGGGGACACG | | AVYFCARVQY | | | |
| | | | | | | | | | | | | | | | | GCTGTGTATTTCTGTGCAAGAGTCCAG | | SSGRHQYSYY | | | |
| | | | | | | | | | | | | | | | | TATAGCAGTGGCCGGCACCAATACTCC | | YMDVWGKGTT | | | |
| | | | | | | | | | | | | | | | | TACTACTACATGGACGTCTGGGGCAAA | | VTVSS | | | |
| | | | | | | | | | | | | | | | | GGGACCACGGTCACCGTCTCCTCA | | | | | |
| ADI- | IGHV3- | QVQLVQTG | 88 | IIVSRNY | 291 | WVRQA | 469 | VLYAGG | 619 | RFTIS | 777 | ARDGVY | 1120 | WGQGTT | 1287 | CAGGTGCAGCTGGTGCAGACTGGAGGA | 1410 | QVQLVQTGGG | 1693 | | |
| 75658 | 53 | GGLIQPGG | | MT | | PGEGL | | STFYAD | | RDNSK | | YGMDI | | VTVSS | | GGCTTGATCCAGCCGGGGGGGTCCCTG | | LIQPGGSLRL | | | |
| | | SLRLSCAA | | | | EWVG | | SVKG | | NTLYL | | | | | | AGACTCTCTTGTGCAGCCTCTGGGATC | | SCAASGIIVS | | | |
| | | SG | | | | | | | | QMNSL | | | | | | ATCGTCAGTCGCAACTACATGACCTGG | | RNYMTWVRQA | | | |
| | | | | | | | | | | RAEDT | | | | | | GTCCGCCAGGCTCCAGGGGAGGGGCTG | | PGEGLEWVGV | | | |
| | | | | | | | | | | AVYYC | | | | | | GAGTGGGTCGGAGTTCTTTATGCCGGT | | LYAGGSTFYA | | | |
| | | | | | | | | | | | | | | | | GGTAGTACATTCTACGCAGACTCCGTG | | DSVKGRFTIS | | | |
| | | | | | | | | | | | | | | | | AAGGGCCGATTCACCATCTCCAGAGAC | | RDNSKNTLYL | | | |
| | | | | | | | | | | | | | | | | AATTCGAAGAACACACTGTATCTTCAA | | QMNSLRAEDT | | | |
| | | | | | | | | | | | | | | | | ATGAACAGCCTGAGAGCCGAGGACACG | | AVYYCARDGV | | | |
| | | | | | | | | | | | | | | | | GCCGTGTATTACTGTGCGAGAGATGGG | | YYGMDIWGQG | | | |
| | | | | | | | | | | | | | | | | GTGTACTATGGGATGGACATCTGGGGC | | TTVTVSS | | | |
| | | | | | | | | | | | | | | | | CAGGGGACCACGGTCACCGTCTCCTCA | | | | | |
| ADI- | IGHV3- | QVQLVESG | 89 | ITVSSNY | 292 | WVRQA | 437 | TIYAGG | 620 | RFTIS | 874 | ARDLEI | 1121 | WGQGTT | 1287 | CAGGTGCAGCTGGTGGAGTCTGGGGGA | 1411 | QVQLVESGGG | 1694 | | |
| 75768 | 66 | GGLVQPGG | | MK | | PGKGL | | STFYAD | | RDNSK | | AGALDI | | VTVSS | | GGCTTGGTCCAGCCTGGGGGGTCCCTG | | LVQPGGSLRL | | | |
| | | SLRLSCAA | | | | EWVS | | SVRG | | NTLYL | | | | | | AGACTCTCCTGTGCAGCCTCTGAAATC | | SCAASEITVS | | | |
| | | SE | | | | | | | | QMNSL | | | | | | ACCGTCAGTAGCAACTACATGAAGTGG | | SNYMKWVRQA | | | |
| | | | | | | | | | | RAGDT | | | | | | GTCCGCCAGGCTCCAGGGAAGGGGCTG | | PGKGLEWVST | | | |
| | | | | | | | | | | AVYYC | | | | | | GAGTGGGTCTCAACTATTTATGCCGGT | | IYAGGSTFYA | | | |
| | | | | | | | | | | | | | | | | GGTAGCACATTCTACGCAGACTCCGTG | | DSVRGRFTIS | | | |
| | | | | | | | | | | | | | | | | AGGGGCAGATTCACCATCTCCAGAGAC | | RDNSKNTLYL | | | |
| | | | | | | | | | | | | | | | | AATTCCAAGAATACACTATATCTTCAA | | QMNSLRAGDT | | | |
| | | | | | | | | | | | | | | | | ATGAACAGCCTGAGAGCCGGAGACACG | | AVYYCARDLE | | | |
| | | | | | | | | | | | | | | | | GCTGTTTATTACTGTGCGAGAGATTTG | | IAGALDIWGQ | | | |
| | | | | | | | | | | | | | | | | GAGATTGCAGGCGCTTTGGATATCTGG | | GTTVTVSS | | | |
| | | | | | | | | | | | | | | | | GGCCAAGGGACCACGGTCACCGTCTCC | | | | | |
| | | | | | | | | | | | | | | | | TCA | | | | | |
| ADI- | IGHV3- | QVQLVQSG | 90 | FTFRNYE | 293 | WVRQA | 470 | YISPSG | 621 | RFTIS | 875 | ARAREL | 1122 | WGQGTL | 1286 | CAGGTCCAGCTGGTGCAGTCTGGGGGA | 1412 | QVQLVQSGGG | 1695 | | |
| 75810 | 48 | GGLVQPGG | | MN | | PGKGL | | VTIHYA | | RDNDK | | VEVFDY | | VTVSS | | GGCTTGGTACAGCCTGGAGGGTCCCTG | | LVQPGGSLRL | | | |
| | | SLRLSCAT | | | | EWIS | | DSVKG | | NSLYL | | | | | | AGACTCTCCTGTGCAACCTCTGGATTC | | SCATSGFTFR | | | |
| | | SG | | | | | | | | QMNSL | | | | | | ACCTTCAGAAATTATGAAATGAATTGG | | NYEMNWVRQA | | | |
| | | | | | | | | | | RGEDT | | | | | | GTCCGCCAGGCTCCAGGGAAGGGGCTG | | PGKGLEWISY | | | |
| | | | | | | | | | | AVYFC | | | | | | GAGTGGATTTCATACATTAGCCCTAGT | | ISPSGVTIHY | | | |
| | | | | | | | | | | | | | | | | GGTGTTACCATACACTACGCAGACTCT | | ADSVKGRFTI | | | |
| | | | | | | | | | | | | | | | | GTGAAGGGCCGATTCACCATCTCCAGA | | SRDNDKNSLY | | | |
| | | | | | | | | | | | | | | | | GACAACGATAAGAATTCACTGTATCTG | | LQMNSLRGED | | | |
| | | | | | | | | | | | | | | | | CAAATGAACAGCCTGAGAGGCGAGGAC | | TAVYFCARAR | | | |
| | | | | | | | | | | | | | | | | ACGGCTGTTTATTTCTGTGCGAGAGCA | | ELVEVFDYWG | | | |
| | | | | | | | | | | | | | | | | AGAGAGCTGGTCGAGGTCTTTGACTAC | | QGTLVTVSS | | | |
| | | | | | | | | | | | | | | | | TGGGGCCAGGGAACCCTGGTCACCGTC | | | | | |
| | | | | | | | | | | | | | | | | TCCTCA | | | | | |
| ADI- | IGHV3- | QVQLVETG | 91 | IIVSRNY | 291 | WVRQV | 436 | VIYPGG | 622 | RFTIS | 876 | AREDNN | 1123 | WGQGTT | 1287 | CAGGTGCAGCTGGTGGAGACTGGAGGA | 1413 | QVQLVETGGG | 1696 | | |
| 75784 | 53 | GGLIQPGG | | MT | | PGKGL | | STFYAD | | RDDSK | | NAFDI | | VTVSS | | GGCTTGATCCAGCCGGGGGGGTCCCTG | | LIQPGGSLRL | | | |
| | | SLRLSCAA | | | | EWVS | | SVRG | | NTLYL | | | | | | AGACTCTCCTGTGCAGCCTCTGAGATC | | SCAASEIIVS | | | |
| | | SE | | | | | | | | QMNSL | | | | | | ATCGTCAGTAGGAACTACATGACCTGG | | RNYMTWVRQV | | | |
| | | | | | | | | | | RAEDT | | | | | | GTCCGCCAGGTCCCAGGGAAAGGGCTG | | PGKGLEWVSV | | | |
| | | | | | | | | | | AVYYC | | | | | | GAGTGGGTCTCAGTTATTTATCCCGGT | | IYPGGSTFYA | | | |
| | | | | | | | | | | | | | | | | GGGAGCACTTTCTACGCAGACTCCGTG | | DSVRGRFTIS | | | |
| | | | | | | | | | | | | | | | | AGGGGCCGATTCACCATCTCCAGAGAC | | RDDSKNTLYL | | | |
| | | | | | | | | | | | | | | | | GATTCCAAGAACACTCTGTATCTTCAA | | QMNSLRAEDT | | | |
| | | | | | | | | | | | | | | | | ATGAACAGCCTGAGAGCCGAGGACACG | | AVYYCAREDN | | | |
| | | | | | | | | | | | | | | | | GCCGTGTATTACTGTGCGAGAGAAGAC | | NNAFDIWGQG | | | |
| | | | | | | | | | | | | | | | | AATAATAATGCTTTTGATATTTGGGGG | | TTVTVSS | | | |
| | | | | | | | | | | | | | | | | CAAGGGACAACGGTCACCGTCTCCTCA | | | | | |
| ADI- | IGHV4- | QVQLQQSG | 92 | GSITSIS | 294 | WVRQP | 444 | EIHHSG | 623 | RLTMS | 877 | ARVSGF | 1048 | WGQGTT | 1287 | CAGGTGCAGCTGCAGCAGTCGGGCCCA | 1414 | QVQLQQSGPG | 1697 | | |
| 75619 | 4 | PGLVKPSG | | WWS | | PGKGL | | STTYNP | | VGKSN | | FDY | | VTVSS | | GGACTGGTGAAGCCTTCGGGGACACTG | | LVKPSGTLSL | | | |
| | | TLSLTCAV | | | | EWIG | | TLRS | | NQFSL | | | | | | TCCCTCACCTGCGCTGTCTCTAGTGGC | | TCAVSSGSIT | | | |
| | | SS | | | | | | | | QLTSV | | | | | | TCCATCACCAGTATTAGCTGGTGGAGT | | SISWWSWVRQ | | | |
| | | | | | | | | | | TAADT | | | | | | TGGGTCCGCCAGCCCCCAGGGAAGGGG | | PPGKGLEWIG | | | |
| | | | | | | | | | | AIYYC | | | | | | CTGGAGTGGATTGGGGAAATCCATCAT | | EIHHSGSTTY | | | |
| | | | | | | | | | | | | | | | | AGTGGGAGTACCACCTACAACCCGACC | | NPTLRSRLTM | | | |
| | | | | | | | | | | | | | | | | CTCAGGAGTCGACTCACCATGTCAGTC | | SVGKSNNQFS | | | |
| | | | | | | | | | | | | | | | | GGCAAGTCCAACAACCAGTTCTCCCTG | | LQLTSVTAAD | | | |
| | | | | | | | | | | | | | | | | CAGCTGACCTCTGTGACCGCCGCGGAC | | TAIYYCARVS | | | |
| | | | | | | | | | | | | | | | | ACGGCCATATATTACTGTGCGAGAGTC | | GFFDYWGQGT | | | |
| | | | | | | | | | | | | | | | | AGTGGATTTTTTGACTATTGGGGCCAG | | TVTVSS | | | |
| | | | | | | | | | | | | | | | | GGGACCACGGTCACCGTCTCCTCA | | | | | |
| ADI- | IGHV4- | QVQLQQWG | 93 | GAFSGYF | 295 | WIRQS | 471 | EVTHSG | 624 | RVTMS | 878 | ARGDFY | 1124 | WGQGSM | 1293 | CAGGTGCAGCTACAGCAGTGGGGCGCA | 1415 | QVQLQQWGAG | 1698 | | |
| 75738 | 34 | AGLLKPSE | | WN | | PVKGL | | NTNYNP | | VDASK | | STRLAF | | VTVSS | | GGACTGCTGAAGCCTTCGGAGACCCTG | | LLKPSETLSL | | | |
| | | TLSLTCGV | | | | EWIG | | SLKS | | NEFSL | | DV | | | | TCCCTCACCTGCGGTGTCCACGATGGG | | TCGVHDGAFS | | | |
| | | HD | | | | | | | | NLTSV | | | | | | GCCTTCAGTGGTTACTTCTGGAACTGG | | GYFWNWIRQS | | | |
| | | | | | | | | | | TAADT | | | | | | ATCCGCCAGTCCCCAGTGAAGGGGCTG | | PVKGLEWIGE | | | |
| | | | | | | | | | | AVYYC | | | | | | GAGTGGATTGGGGAAGTCACTCATAGT | | VTHSGNTNYN | | | |
| | | | | | | | | | | | | | | | | GGCAACACCAACTACAACCCGTCCCTC | | PSLKSRVTMS | | | |
| | | | | | | | | | | | | | | | | AAGAGTCGAGTCACCATGTCAGTAGAC | | VDASKNEFSL | | | |
| | | | | | | | | | | | | | | | | GCGTCCAAGAACGAATTCTCCCTGAAC | | NLTSVTAADT | | | |
| | | | | | | | | | | | | | | | | CTGACCTCTGTGACCGCCGCGGACACG | | AVYYCARGDF | | | |
| | | | | | | | | | | | | | | | | GCTGTGTATTACTGTGCGAGGGGCGAC | | YSTRLAFDVW | | | |
| | | | | | | | | | | | | | | | | TTTTATAGCACCAGGCTCGCTTTTGAT | | GQGSMVTVSS | | | |
| | | | | | | | | | | | | | | | | GTCTGGGGCCAAGGGTCAATGGTCACC | | | | | |
| | | | | | | | | | | | | | | | | GTCTCCTCA | | | | | |
| ADI- | IGHV2- | QVQLQQSG | 94 | FSLNTGG | 296 | WIRQP | 441 | LIYWDD | 625 | RLAIT | 879 | ARLKIA | 1125 | WGQGTL | 1286 | CAGGTCCAGCTGCAGCAGTCTGGTCCT | 1416 | QVQLQQSGPT | 1699 | | |
| 75790 | 5 | PTLVKPTQ | | VAVG | | PGKAL | | DKRYSP | | KDTSK | | PIVDA | | VTVSS | | ACGCTGGTGAAACCCACACAGACCCTC | | LVKPTQTLTL | | | |
| | | TLTLTCTF | | | | EWLA | | SLKN | | NQVVL | | | | | | ACGCTGACCTGCACCTTCTCTGGGTTC | | TCTFSGFSLN | | | |
| | | SG | | | | | | | | TMTDM | | | | | | TCCCTCAACACTGGTGGAGTGGCTGTG | | TGGVAVGWIR | | | |
| | | | | | | | | | | DPVDT | | | | | | GGCTGGATCCGTCAGCCCCCAGGAAAG | | QPPGKALEWL | | | |
| | | | | | | | | | | ATYYC | | | | | | GCCCTGGAGTGGCTTGCACTCATTTAT | | ALIYWDDDKR | | | |
| | | | | | | | | | | | | | | | | TGGGATGATGATAAGCGCTACAGCCCA | | YSPSLKNRLA | | | |
| | | | | | | | | | | | | | | | | TCTCTGAAGAACAGGCTCGCCATCACC | | ITKDTSKNQV | | | |
| | | | | | | | | | | | | | | | | AAGGACACCTCCAAAAACCAGGTGGTC | | VLTMTDMDPV | | | |
| | | | | | | | | | | | | | | | | CTTACAATGACCGACATGGACCCTGTG | | DTATYYCARL | | | |
| | | | | | | | | | | | | | | | | GACACAGCCACATATTACTGTGCACGC | | KIAPIVDAWG | | | |
| | | | | | | | | | | | | | | | | CTTAAGATAGCTCCAATAGTTGACGCC | | QGTLVTVSS | | | |
| | | | | | | | | | | | | | | | | TGGGGCCAGGGAACCCTGGTCACCGTC | | | | | |
| | | | | | | | | | | | | | | | | TCCTCA | | | | | |
| ADI- | IGHV3- | QVQLVQSG | 10 | FAFTTYV | 297 | WVRQA | 472 | VISSDG | 626 | RFTIS | 880 | ARDGGY | 1126 | WGQGTT | 1287 | CAGGTGCAGCTGGTGCAGTCTGGGGGA | 1417 | QVQLVQSGGG | 1700 | | |
| 75719 | 30 | GGVVQPGR | | MH | | PGKGL | | SAKFYA | | RDSSQ | | SGYFDA | | VTVSS | | GGCGTGGTCCAGCCTGGGAGGTCCCTG | | VVQPGRSLRL | | | |
| | | SLRLSCAA | | | | EWVT | | DSVKG | | NTLYL | | FDI | | | | AGACTCTCCTGTGCAGCCTCTGGATTC | | SCAASGFAFT | | | |
| | | SG | | | | | | | | QMNSL | | | | | | GCCTTCACTACCTATGTTATGCACTGG | | TYVMHWVRQA | | | |
| | | | | | | | | | | RAEDT | | | | | | GTCCGCCAGGCTCCAGGCAAGGGGCTG | | PGKGLEWVTV | | | |
| | | | | | | | | | | AVYYC | | | | | | GAGTGGGTAACCGTTATCTCATCTGAT | | ISSDGSAKFY | | | |
| | | | | | | | | | | | | | | | | GGAAGTGCTAAATTTTACGCAGACTCC | | ADSVKGRFTI | | | |
| | | | | | | | | | | | | | | | | GTGAAGGGCCGATTCACCATCTCCAGA | | SRDSSQNTLY | | | |
| | | | | | | | | | | | | | | | | GACAGTTCCCAGAATACGCTCTATCTT | | LQMNSLRAED | | | |
| | | | | | | | | | | | | | | | | CAAATGAACAGCCTGAGAGCTGAGGAC | | TAVYYCARDG | | | |
| | | | | | | | | | | | | | | | | ACGGCTGTCTATTACTGTGCGAGAGAT | | GYSGYFDAFD | | | |
| | | | | | | | | | | | | | | | | GGGGGATATAGTGGCTACTTTGATGCC | | IWGQGTTVTV | | | |
| | | | | | | | | | | | | | | | | TTTGATATCTGGGGCCAAGGGACCACG | | SS | | | |
| | | | | | | | | | | | | | | | | GTCACCGTCTCCTCA | | | | | |
| ADI- | IGHV1- | QVQLVQSG | 95 | GTFSNYF | 298 | WVRQA | 473 | RIIPIL | 627 | RVTIT | 832 | AREGGT | 1127 | WGQGTL | 1286 | CAGGTGCAGCTGGTGCAGTCTGGGGCT | 1418 | QVQLVQSGAE | 1701 | | |
| 75742 | 69 | AEVKKLGS | | IS | | PGQGL | | GIAYYA | | ADKST | | TTEYFQ | | VTVSS | | GAAGTGAAGAAGCTTGGGTCCTCGGTG | | VKKLGSSVKV | | | |
| | | SVKVSCKA | | | | ECMG | | QKFQG | | STAYM | | H | | | | AAGGTCTCCTGCAAGGCCTCTGGAGGC | | SCKASGGTFS | | | |
| | | SG | | | | | | | | ELSSL | | | | | | ACGTTCAGCAACTATTTTATCAGCTGG | | NYFISWVRQA | | | |
| | | | | | | | | | | RSEDT | | | | | | GTGCGACAGGCCCCTGGACAAGGGCTT | | PGQGLECMGR | | | |
| | | | | | | | | | | AVYYC | | | | | | GAGTGCATGGGGAGGATCATCCCTATC | | IIPILGIAYY | | | |
| | | | | | | | | | | | | | | | | CTTGGTATAGCATACTACGCTCAGAAG | | AQKFQGRVTI | | | |
| | | | | | | | | | | | | | | | | TTCCAGGGCAGAGTCACGATTACCGCG | | TADKSTSTAY | | | |
| | | | | | | | | | | | | | | | | GACAAATCCACGAGCACAGCCTACATG | | MELSSLRSED | | | |
| | | | | | | | | | | | | | | | | GAGCTGAGCAGCCTGAGATCTGAGGAC | | TAVYYCAREG | | | |
| | | | | | | | | | | | | | | | | ACGGCCGTGTATTATTGTGCGAGAGAA | | GTTTEYFQHW | | | |
| | | | | | | | | | | | | | | | | GGTGGTACCACCACTGAATACTTCCAG | | GQGTLVTVSS | | | |
| | | | | | | | | | | | | | | | | CATTGGGGCCAGGGCACCCTGGTCACC | | | | | |
| | | | | | | | | | | | | | | | | GTCTCCTCA | | | | | |
| ADI- | IGHV3- | QVQLVQSG | 96 | FTFSSYG | 262 | WVRQA | 431 | VIWYNG | 628 | RFTIS | 777 | ARDTWE | 1128 | WGRGTT | 1294 | CAGGTGCAGCTGGTGCAGTCTGGGGGA | 1419 | QVQLVQSGGD | 1702 | | |
| 75779 | 33 | GDVVQPGR | | MH | | PGKGL | | SNKYYI | | RDNSK | | VPVADP | | VTVSS | | GACGTGGTCCAGCCTGGGAGGTCCCTG | | VVQPGRSLRL | | | |
| | | SLRLSCAT | | | | EWVA | | DSVKG | | NTLYL | | PYYYYY | | | | AGACTCTCCTGTGCAACGTCTGGATTC | | SCATSGFTFS | | | |
| | | SG | | | | | | | | QMNSL | | MDV | | | | ACCTTCAGTAGCTATGGCATGCACTGG | | SYGMHWVRQA | | | |
| | | | | | | | | | | RAEDT | | | | | | GTCCGCCAGGCTCCAGGCAAGGGGCTG | | PGKGLEWVAV | | | |
| | | | | | | | | | | AVYYC | | | | | | GAGTGGGTGGCAGTTATTTGGTATAAT | | IWYNGSNKYY | | | |
| | | | | | | | | | | | | | | | | GGAAGTAATAAATACTATATAGACTCC | | IDSVKGRFTI | | | |
| | | | | | | | | | | | | | | | | GTGAAGGGCCGATTCACCATCTCCAGA | | SRDNSKNTLY | | | |
| | | | | | | | | | | | | | | | | GACAATTCCAAGAACACGCTCTATCTG | | LQMNSLRAED | | | |
| | | | | | | | | | | | | | | | | CAAATGAACAGCCTGAGAGCCGAGGAC | | TAVYYCARDT | | | |
| | | | | | | | | | | | | | | | | ACGGCTGTGTATTACTGTGCGAGAGAT | | WEVPVADPPY | | | |
| | | | | | | | | | | | | | | | | ACTTGGGAAGTACCAGTTGCCGACCCT | | YYYYMDVWGR | | | |
| | | | | | | | | | | | | | | | | CCTTATTATTATTACTACATGGACGTG | | GTTVTVSS | | | |
| | | | | | | | | | | | | | | | | TGGGGCAGAGGGACCACGGTCACCGTC | | | | | |
| | | | | | | | | | | | | | | | | TCCTCA | | | | | |
| ADI- | IGHV1- | QVQLVQSG | 97 | YTFTSYY | 299 | WVRQA | 433 | IINPSD | 629 | RVTMT | 881 | ARNSFG | 1129 | WGQGTL | 1286 | CAGGTCCAGCTGGTGCAGTCTGGGGCT | 1420 | QVQLVQSGAE | 1703 | | |
| 75650 | 46 | AEVKKPGA | | MH | | PGQGL | | AGASYV | | RDTST | | YYYDNS | | VTVSS | | GAAGTGAAGAAGCCTGGGGCCTCAATG | | VKKPGASMKV | | | |
| | | SMKVSCKA | | | | EWMG | | QKFQG | | STVYM | | AYLPAL | | | | AAGGTTTCATGCAAGGCATATGGATAC | | SCKAYGYTFT | | | |
| | | YG | | | | | | | | ELSSL | | DFDY | | | | ACTTTCACCAGCTACTATATGCATTGG | | SYYMHWVRQA | | | |
| | | | | | | | | | | RSEDT | | | | | | GTGCGACAGGCCCCTGGACAAGGGCTT | | PGQGLEWMGI | | | |
| | | | | | | | | | | AVYYC | | | | | | GAGTGGATGGGAATAATCAACCCTAGT | | INPSDAGASY | | | |
| | | | | | | | | | | | | | | | | GATGCTGGCGCAAGCTACGTACAGAAG | | VQKFQGRVTM | | | |
| | | | | | | | | | | | | | | | | TTCCAGGGCAGAGTCACCATGACCAGG | | TRDTSTSTVY | | | |
| | | | | | | | | | | | | | | | | GACACGTCCACGAGCACAGTCTACATG | | MELSSLRSED | | | |
| | | | | | | | | | | | | | | | | GAGCTGAGCAGCCTGAGATCTGAGGAC | | TAVYYCARNS | | | |
| | | | | | | | | | | | | | | | | ACGGCCGTGTACTACTGTGCGAGAAAT | | FGYYYDNSAY | | | |
| | | | | | | | | | | | | | | | | TCTTTTGGATATTACTATGATAATAGT | | LPALDFDYWG | | | |
| | | | | | | | | | | | | | | | | GCTTATTTGCCCGCCTTGGACTTTGAC | | QGTLVTVSS | | | |
| | | | | | | | | | | | | | | | | TACTGGGGCCAGGGAACCCTGGTCACC | | | | | |
| | | | | | | | | | | | | | | | | GTCTCCTCA | | | | | |
| ADI- | IGHV3- | QVQLQQSG | 98 | ITVSSNY | 300 | WVRQA | 437 | IIYPGG | 608 | RFSIS | 882 | ARDRGG | 1108 | WGQGTL | 1286 | CAGGTGCAGCTGCAGCAGTCTGGAGGA | 1421 | QVQLQQSGGG | 1704 | | |
| 75593 | 53 | GGLIQPGG | | MS | | PGKGL | | SSFYAE | | RDSSK | | YPDY | | VTVSS | | GGCTTGATCCAGCCTGGGGGGTCCCTG | | LIQPGGSLRL | | | |
| | | SLRLSCVA | | | | EWVS | | SVKG | | NTLYL | | | | | | AGACTCTCCTGTGTAGCCTCTGAGATC | | SCVASEITVS | | | |
| | | SE | | | | | | | | QMDSL | | | | | | ACCGTCAGTAGCAACTACATGAGCTGG | | SNYMSWVRQA | | | |
| | | | | | | | | | | TVEDT | | | | | | GTCCGCCAGGCTCCAGGGAAGGGACTG | | PGKGLEWVSI | | | |
| | | | | | | | | | | AIYYC | | | | | | GAGTGGGTCTCAATAATTTATCCCGGT | | IYPGGSSFYA | | | |
| | | | | | | | | | | | | | | | | GGTAGTTCATTCTACGCAGAGTCCGTG | | ESVKGRFSIS | | | |
| | | | | | | | | | | | | | | | | AAGGGCCGATTCAGCATCTCCAGGGAC | | RDSSKNTLYL | | | |
| | | | | | | | | | | | | | | | | AGTTCCAAGAACACCCTCTATCTTCAA | | QMDSLTVEDT | | | |
| | | | | | | | | | | | | | | | | ATGGACAGCCTGACAGTCGAGGACACG | | AIYYCARDRG | | | |
| | | | | | | | | | | | | | | | | GCCATTTACTATTGTGCGAGAGATCGG | | GYPDYWGQGT | | | |
| | | | | | | | | | | | | | | | | GGTGGGTACCCTGACTACTGGGGCCAG | | LVTVSS | | | |
| | | | | | | | | | | | | | | | | GGAACCCTGGTCACCGTCTCCTCA | | | | | |
| ADI- | IGHV1- | QVQLVQSG | 35 | HTFANYY | 301 | WVRQA | 433 | IINPSG | 630 | RVTMT | 881 | ARGGIV | 1130 | WGQGTL | 1286 | CAGGTGCAGCTGGTGCAGTCTGGGGCT | 1422 | QVQLVQSGAE | 1705 | | |
| 75814 | 46 | AEVKKPGA | | MH | | PGQGL | | GSTAYA | | RDTST | | PDAKQY | | VTVSS | | GAGGTGAAGAAACCTGGGGCCTCAGTG | | VKKPGASVKV | | | |
| | | SVKVSCKA | | | | EWMG | | QHFQD | | STVYM | | FDY | | | | AAGGTTTCCTGCAAGGCATCCGGACAC | | SCKASGHTFA | | | |
| | | SG | | | | | | | | ELSSL | | | | | | ACCTTCGCCAACTACTATATGCACTGG | | NYYMHWVRQA | | | |
| | | | | | | | | | | RSEDT | | | | | | GTGCGACAGGCCCCTGGACAAGGGCTT | | PGQGLEWMGI | | | |
| | | | | | | | | | | AVYYC | | | | | | GAGTGGATGGGCATAATCAACCCTAGT | | INPSGGSTAY | | | |
| | | | | | | | | | | | | | | | | GGTGGTAGCACAGCCTACGCACAGCAC | | AQHFQDRVTM | | | |
| | | | | | | | | | | | | | | | | TTCCAGGACAGAGTCACCATGACCAGG | | TRDTSTSTVY | | | |
| | | | | | | | | | | | | | | | | GACACGTCCACGAGCACAGTCTACATG | | MELSSLRSED | | | |
| | | | | | | | | | | | | | | | | GAGCTGAGCAGCCTGAGATCTGAGGAC | | TAVYYCARGG | | | |
| | | | | | | | | | | | | | | | | ACGGCCGTGTATTACTGTGCGAGAGGG | | IVPDAKQYFD | | | |
| | | | | | | | | | | | | | | | | GGTATAGTCCCAGATGCTAAGCAATAC | | YWGQGTLVTV | | | |
| | | | | | | | | | | | | | | | | TTTGACTACTGGGGCCAGGGAACCCTG | | SS | | | |
| | | | | | | | | | | | | | | | | GTCACCGTCTCCTCA | | | | | |
| ADI- | IGHV3- | QVQLVESG | 11 | FTFSTYG | 215 | WVRQA | 431 | VISYDG | 631 | RFTIS | 883 | AKEGPG | 1073 | WGQGTL | 1286 | CAGGTGCAGCTGGTGGAGTCTGGGGGA | 1423 | QVQLVESGGG | 1706 | | |
| 75662 | 30 | GGVVQPGR | | MH | | PGKGL | | SNKHYA | | RDNSK | | ASGFNY | | VTVSS | | GGCGTAGTCCAGCCTGGGAGGTCCCTG | | VVQPGRSLRL | | | |
| | | SLRLSCAA | | | | EWVA | | DSVKG | | KTLYL | | FDY | | | | AGACTCTCCTGTGCAGCCTCTGGATTC | | SCAASGFTFS | | | |
| | | SG | | | | | | | | QMNSL | | | | | | ACCTTCAGTACCTATGGCATGCACTGG | | TYGMHWVRQA | | | |
| | | | | | | | | | | RTEDA | | | | | | GTCCGCCAGGCTCCAGGCAAGGGGCTG | | PGKGLEWVAV | | | |
| | | | | | | | | | | AVYYC | | | | | | GAGTGGGTGGCAGTTATATCATATGAT | | ISYDGSNKHY | | | |
| | | | | | | | | | | | | | | | | GGAAGTAATAAACACTATGCAGACTCC | | ADSVKGRFTI | | | |
| | | | | | | | | | | | | | | | | GTGAAGGGCCGATTCACCATCTCCAGA | | SRDNSKKTLY | | | |
| | | | | | | | | | | | | | | | | GACAATTCCAAGAAGACGCTGTATCTG | | LQMNSLRTED | | | |
| | | | | | | | | | | | | | | | | CAAATGAACAGCCTGAGAACTGAGGAC | | AAVYYCAKEG | | | |
| | | | | | | | | | | | | | | | | GCGGCTGTGTATTACTGTGCGAAAGAG | | PGASGFNYFD | | | |
| | | | | | | | | | | | | | | | | GGGCCCGGGGCTTCGGGATTTAACTAC | | YWGQGTLVTV | | | |
| | | | | | | | | | | | | | | | | TTTGACTACTGGGGCCAGGGAACCCTG | | SS | | | |
| | | | | | | | | | | | | | | | | GTCACCGTCTCCTCA | | | | | |
| ADI- | IGHV3- | EVQLVQTG | 99 | FIVSRNY | 302 | WVRQA | 437 | LIYAGG | 632 | RFTIS | 789 | TRDRGP | 1131 | WGQGTL | 1286 | GAGGTGCAGCTGGTGCAGACTGGAGGA | 1424 | EVQLVQTGGG | 1707 | | |
| 75627 | 53 | GGLIQPGG | | MS | | PGKGL | | STFYAD | | RDNSK | | LLHDY | | VTVSS | | GGCTTGATCCAGCCTGGGGGGTCCCTG | | LIQPGGSLRL | | | |
| | | SLRLSCAA | | | | EWVS | | SVKG | | NTLYL | | | | | | AGACTCTCCTGTGCAGCCTCTGAATTC | | SCAASEFIVS | | | |
| | | SE | | | | | | | | QMNSL | | | | | | ATCGTCAGTCGCAACTACATGAGTTGG | | RNYMSWVRQA | | | |
| | | | | | | | | | | RTEDT | | | | | | GTCCGCCAGGCTCCAGGGAAGGGGCTG | | PGKGLEWVSL | | | |
| | | | | | | | | | | AVYYC | | | | | | GAGTGGGTCTCACTTATCTATGCCGGT | | IYAGGSTFYA | | | |
| | | | | | | | | | | | | | | | | GGTAGTACATTCTACGCAGACTCCGTG | | DSVKGRFTIS | | | |
| | | | | | | | | | | | | | | | | AAGGGCCGATTCACCATCTCCAGAGAC | | RDNSKNTLYL | | | |
| | | | | | | | | | | | | | | | | AATTCCAAGAACACACTTTATCTACAA | | QMNSLRTEDT | | | |
| | | | | | | | | | | | | | | | | ATGAACAGCCTCAGAACCGAGGACACG | | AVYYCTRDRG | | | |
| | | | | | | | | | | | | | | | | GCCGTGTATTACTGTACGAGAGATAGA | | PLLHDYWGQG | | | |
| | | | | | | | | | | | | | | | | GGGCCTTTACTACACGACTACTGGGGC | | TLVTVSS | | | |
| | | | | | | | | | | | | | | | | CAGGGAACCCTGGTCACCGTCTCCTCA | | | | | |
| ADI- | IGHV3- | QVQLVESG | 100 | FTFEDYA | 208 | WVRQT | 474 | GISWNG | 633 | RFIIS | 884 | TKAQAP | 1132 | WGPGTT | 1295 | CAGGTGCAGCTGGTGGAGTCTGGGGGA | 1425 | QVQLVESGGG | 1708 | | |
| 75792 | 9 | GGLVQPGR | | MH | | PGKGP | | GIMGYA | | RDNAK | | LDSGDY | | VTVSS | | GGCTTGGTACAGCCTGGCAGGTCCCTG | | LVQPGRSLRL | | | |
| | | SLRLSCAA | | | | EWVS | | ASVEG | | NSLYL | | GGGFHI | | | | AGACTCTCCTGTGCAGCCTCTGGATTC | | SCAASGFTFE | | | |
| | | SG | | | | | | | | QMMRL | | | | | | ACCTTTGAAGATTATGCCATGCACTGG | | DYAMHWVRQT | | | |
| | | | | | | | | | | RAEDT | | | | | | GTCCGGCAAACTCCAGGGAAGGGCCCG | | PGKGPEWVSG | | | |
| | | | | | | | | | | AFYYC | | | | | | GAGTGGGTCTCAGGTATTAGTTGGAAT | | ISWNGGIMGY | | | |
| | | | | | | | | | | | | | | | | GGGGGTATCATGGGCTATGCGGCCTCT | | AASVEGRFII | | | |
| | | | | | | | | | | | | | | | | GTAGAGGGCCGATTCATCATCTCCAGA | | SRDNAKNSLY | | | |
| | | | | | | | | | | | | | | | | GACAACGCCAAGAACTCCCTGTATCTG | | LQMMRLRAED | | | |
| | | | | | | | | | | | | | | | | CAAATGATGCGTCTGAGAGCAGAGGAC | | TAFYYCTKAQ | | | |
| | | | | | | | | | | | | | | | | ACGGCCTTCTATTACTGTACAAAAGCC | | APLDSGDYGG | | | |
| | | | | | | | | | | | | | | | | CAAGCCCCACTTGACTCCGGTGACTAC | | GFHIWGPGTT | | | |
| | | | | | | | | | | | | | | | | GGCGGTGGTTTTCATATCTGGGGCCCA | | VTVSS | | | |
| | | | | | | | | | | | | | | | | GGGACAACGGTCACCGTCTCTTCA | | | | | |
| ADI- | IGHV3- | QVQLVESG | 101 | FTFRDHA | 303 | WVRQA | 475 | TISGSG | 634 | RFTIS | 885 | ARGTTI | 1133 | WGQGTL | 1286 | CAGGTGCAGCTGGTGGAGTCTGGGGGA | 1426 | QVQLVESGGG | 1709 | | |
| 75791 | 23 | GGLVQPGG | | MS | | PGKGL | | DTFSHA | | RDNSK | | FGGALD | | VTVSS | | GGCTTGGTACAGCCTGGGGGGTCCCTG | | LVQPGGSLRV | | | |
| | | SLRVSCAG | | | | DWAS | | DSVKG | | DTLYL | | IP | | | | AGAGTCTCCTGTGCAGGCTCTGGATTC | | SCAGSGFTFR | | | |
| | | SG | | | | | | | | QMTSL | | | | | | ACCTTTAGAGACCACGCCATGAGCTGG | | DHAMSWVRQA | | | |
| | | | | | | | | | | RGEDT | | | | | | GTCCGCCAGGCTCCAGGGAAGGGACTG | | PGKGLDWAST | | | |
| | | | | | | | | | | AVYFC | | | | | | GACTGGGCCTCAACTATCAGTGGCAGT | | ISGSGDTFSH | | | |
| | | | | | | | | | | | | | | | | GGTGATACTTTTAGCCACGCAGATTCC | | ADSVKGRFTI | | | |
| | | | | | | | | | | | | | | | | GTGAAGGGCCGGTTCACCATCTCCAGA | | SRDNSKDTLY | | | |
| | | | | | | | | | | | | | | | | GACAACTCCAAGGACACTCTCTACTTA | | LQMTSLRGED | | | |
| | | | | | | | | | | | | | | | | CAAATGACCAGCCTGAGAGGCGAGGAC | | TAVYFCARGT | | | |
| | | | | | | | | | | | | | | | | ACGGCCGTGTATTTCTGTGCGAGAGGG | | TIFGGALDPW | | | |
| | | | | | | | | | | | | | | | | ACCACGATTTTTGGGGGGGCTTTAGAC | | GQGTLVTVSS | | | |
| | | | | | | | | | | | | | | | | CCCTGGGGCCAGGGAACCCTGGTCACC | | | | | |
| | | | | | | | | | | | | | | | | GTCTCTTCA | | | | | |
| ADI- | IGHV5- | QVQLVQSG | 102 | YYFARYW | 304 | WVRQM | 476 | IIYPGD | 635 | QVTMS | 886 | ARQGTT | 1134 | WGQGTL | 1286 | CAGGTCCAGCTGGTGCAGTCTGGAGCA | 1427 | QVQLVQSGAE | 1710 | | |
| 75721 | 51 | AEVKKAGE | | IG | | PGKGL | | SDTRYS | | ADKSI | | VGHFDY | | VTVSS | | GAGGTGAAAAAGGCCGGGGAGTCTCTG | | VKKAGESLKI | | | |
| | | SLKISCKG | | | | EWMG | | PSFQG | | STAFL | | | | | | AAGATCTCCTGTAAGGGTTCTGGATAC | | SCKGSGYYFA | | | |
| | | SG | | | | | | | | QWSSL | | | | | | TACTTTGCCAGATACTGGATCGGCTGG | | RYWIGWVRQM | | | |
| | | | | | | | | | | KASDT | | | | | | GTGCGCCAGATGCCCGGGAAAGGCCTG | | PGKGLEWMGI | | | |
| | | | | | | | | | | AMYYC | | | | | | GAATGGATGGGGATCATCTATCCTGGT | | IYPGDSDTRY | | | |
| | | | | | | | | | | | | | | | | GACTCGGATACCAGATACAGCCCGTCC | | SPSFQGQVTM | | | |
| | | | | | | | | | | | | | | | | TTCCAAGGCCAGGTCACCATGTCAGCC | | SADKSISTAF | | | |
| | | | | | | | | | | | | | | | | GACAAGTCCATCAGCACCGCCTTCCTG | | LQWSSLKASD | | | |
| | | | | | | | | | | | | | | | | CAGTGGAGCAGCCTGAAGGCCTCGGAC | | TAMYYCARQG | | | |
| | | | | | | | | | | | | | | | | ACCGCCATGTATTACTGTGCGAGACAG | | TTVGHFDYWG | | | |
| | | | | | | | | | | | | | | | | GGCACGACAGTAGGACACTTTGACTAC | | QGTLVTVSS | | | |
| | | | | | | | | | | | | | | | | TGGGGCCAGGGAACCCTGGTCACCGTC | | | | | |
| | | | | | | | | | | | | | | | | TCCTCA | | | | | |
| ADI- | IGHV1- | QVQLQQSG | 103 | YTFSNYA | 305 | WVRQA | 477 | WINPAN | 636 | RVTFT | 887 | ARGVSP | 1135 | WGRGTT | 1294 | CAGGTACAGCTGCAGCAGTCTGGGGCT | 1428 | QVQLQQSGAE | 1711 | | |
| 75761 | 3 | AEVKKPGA | | IH | | PGQGP | | NNIKYS | | RDTSA | | NTVVGL | | VTVSS | | GAGGTGAAGAAGCCTGGGGCCTCAGTG | | VKKPGASVKV | | | |
| | | SVKVSCKA | | | | EWMG | | EKFQG | | STGYM | | GYYYYM | | | | AAAGTTTCCTGCAAGGCTTCTGGATAC | | SCKASGYTFS | | | |
| | | SG | | | | | | | | EMGSL | | DV | | | | ACCTTCTCTAACTATGCAATACATTGG | | NYAIHWVRQA | | | |
| | | | | | | | | | | TSEDT | | | | | | GTGCGCCAGGCCCCCGGACAAGGGCCT | | PGQGPEWMGW | | | |
| | | | | | | | | | | AVYYC | | | | | | GAGTGGATGGGGTGGATCAACCCTGCC | | INPANNNIKY | | | |
| | | | | | | | | | | | | | | | | AATAATAACATAAAGTATTCAGAGAAG | | SEKFQGRVTF | | | |
| | | | | | | | | | | | | | | | | TTCCAGGGCAGAGTCACCTTTACCAGG | | TRDTSASTGY | | | |
| | | | | | | | | | | | | | | | | GACACATCCGCGAGCACAGGCTACATG | | MEMGSLTSED | | | |
| | | | | | | | | | | | | | | | | GAGATGGGAAGCCTGACATCTGAAGAC | | TAVYYCARGV | | | |
| | | | | | | | | | | | | | | | | ACGGCTGTCTATTACTGTGCGAGAGGT | | SPNTVVGLGY | | | |
| | | | | | | | | | | | | | | | | GTCTCCCCTAATACGGTTGTTGGATTG | | YYYMDVWGRG | | | |
| | | | | | | | | | | | | | | | | GGTTACTACTACTACATGGACGTCTGG | | TTVTVSS | | | |
| | | | | | | | | | | | | | | | | GGCAGAGGGACCACGGTCACCGTCTCC | | | | | |
| | | | | | | | | | | | | | | | | TCA | | | | | |
| ADI- | IGHV1- | QVQLVQSG | 50 | GTFIRNT | 306 | WLRQA | 478 | RVIPLL | 637 | RVTII | 888 | AREGTG | 1136 | WGQGTT | 1287 | CAGGTGCAGCTGGTGCAGTCTGGGGCT | 1429 | QVQLVQSGAE | 1712 | | |
| 75682 | 69 | AEVKKPGS | | IS | | PGQGL | | NVVDYA | | ADKSS | | YCDGGV | | VTVSS | | GAGGTGAAGAAGCCTGGGTCCTCGGTG | | VKKPGSSVKV | | | |
| | | SVKVSCKA | | | | EWMG | | QKFQG | | STVYM | | CHDAFE | | | | AAGGTCTCCTGCAAGGCTTCTGGAGGC | | SCKASGGTFI | | | |
| | | SG | | | | | | | | ELSSL | | I | | | | ACCTTCATCAGGAACACTATAAGTTGG | | RNTISWLRQA | | | |
| | | | | | | | | | | RSEDT | | | | | | CTGCGACAGGCCCCTGGACAGGGGCTT | | PGQGLEWMGR | | | |
| | | | | | | | | | | AVYYC | | | | | | GAGTGGATGGGAAGGGTCATCCCTTTA | | VIPLLNVVDY | | | |
| | | | | | | | | | | | | | | | | CTTAATGTAGTTGACTACGCGCAGAAG | | AQKFQGRVTI | | | |
| | | | | | | | | | | | | | | | | TTCCAGGGCAGAGTCACGATAATCGCG | | IADKSSSTVY | | | |
| | | | | | | | | | | | | | | | | GACAAATCTTCGAGCACAGTCTACATG | | MELSSLRSED | | | |
| | | | | | | | | | | | | | | | | GAGTTGAGTAGCCTGAGATCTGAGGAC | | TAVYYCAREG | | | |
| | | | | | | | | | | | | | | | | ACGGCCGTGTATTACTGTGCGAGAGAG | | TGYCDGGVCH | | | |
| | | | | | | | | | | | | | | | | GGGACCGGGTATTGTGATGGTGGTGTA | | DAFEIWGQGT | | | |
| | | | | | | | | | | | | | | | | TGCCATGATGCATTTGAGATCTGGGGC | | TVTVSS | | | |
| | | | | | | | | | | | | | | | | CAAGGGACAACGGTCACCGTCTCCTCA | | | | | |
| ADI- | IGHV3- | QVQLVQSG | 79 | ITVSSNY | 300 | WVRQA | 437 | LLYAGG | 638 | RFTIS | 889 | ARDLVD | 1137 | WGQGTT | 1287 | CAGGTGCAGCTGGTGCAGTCTGGAGGA | 1430 | QVQLVQSGGG | 1713 | | |
| 75847 | 53 | GGLIQPGG | | MS | | PGKGL | | STFYAD | | RDNSK | | YGMDV | | VTVSS | | GGCTTGATCCAGCCGGGGGGGTCCCTG | | LIQPGGSLRL | | | |
| | | SLRLSCAA | | | | EWVS | | SVKG | | NTLFL | | | | | | AGACTCTCCTGCGCAGCCTCTGGGATC | | SCAASGITVS | | | |
| | | SG | | | | | | | | QMNSL | | | | | | ACCGTCAGTAGCAACTACATGAGCTGG | | SNYMSWVRQA | | | |
| | | | | | | | | | | RADDT | | | | | | GTCCGCCAGGCTCCAGGGAAGGGGCTG | | PGKGLEWVSL | | | |
| | | | | | | | | | | AVYYC | | | | | | GAGTGGGTCTCACTTCTTTATGCCGGT | | LYAGGSTFYA | | | |
| | | | | | | | | | | | | | | | | GGAAGCACCTTCTACGCAGACTCCGTG | | DSVKGRFTIS | | | |
| | | | | | | | | | | | | | | | | AAGGGCCGATTCACCATCTCCAGAGAC | | RDNSKNTLFL | | | |
| | | | | | | | | | | | | | | | | AATTCCAAGAACACACTGTTTCTTCAA | | QMNSLRADDT | | | |
| | | | | | | | | | | | | | | | | ATGAACAGCCTGAGAGCCGACGACACG | | AVYYCARDLV | | | |
| | | | | | | | | | | | | | | | | GCCGTGTATTACTGTGCGAGAGATCTG | | DYGMDVWGQG | | | |
| | | | | | | | | | | | | | | | | GTGGATTACGGTATGGACGTCTGGGGC | | TTVTVSS | | | |
| | | | | | | | | | | | | | | | | CAAGGGACCACGGTCACCGTCTCCTCA | | | | | |
| ADI- | IGHV3- | QVQLVESG | 104 | FTFSSYE | 307 | WVRQA | 479 | SISSRG | 639 | RFTIS | 890 | ARDLGG | 1138 | WGQGTL | 1286 | CAGGTGCAGCTGGTGGAGTCTGGGGGA | 1431 | QVQLVESGGG | 1714 | | |
| 75852 | 48 | GGLVQPGG | | MN | | PGKGL | | SSKFYA | | RDNAK | | SYSGTF | | VTVSS | | GGCTTGGTACAGCCTGGAGGGTCCCTG | | LVQPGGSLRL | | | |
| | | SLRLYCAA | | | | ESIS | | DSVKG | | NSVFL | | DH | | | | AGACTCTATTGTGCAGCCTCTGGATTC | | YCAASGFTFS | | | |
| | | SG | | | | | | | | QMNSL | | | | | | ACCTTCAGTAGTTATGAAATGAATTGG | | SYEMNWVRQA | | | |
| | | | | | | | | | | RVEDT | | | | | | GTCCGCCAGGCTCCTGGGAAGGGGCTG | | PGKGLESISS | | | |
| | | | | | | | | | | ALYYC | | | | | | GAATCGATCTCATCCATTAGTAGTCGT | | ISSRGSSKFY | | | |
| | | | | | | | | | | | | | | | | GGGAGTTCCAAATTCTACGCAGACTCT | | ADSVKGRFTI | | | |
| | | | | | | | | | | | | | | | | GTGAAGGGCCGATTCACCATCTCCAGA | | SRDNAKNSVF | | | |
| | | | | | | | | | | | | | | | | GACAACGCCAAGAACTCGGTGTTTCTG | | LQMNSLRVED | | | |
| | | | | | | | | | | | | | | | | CAAATGAACAGCCTGAGAGTCGAGGAC | | TALYYCARDL | | | |
| | | | | | | | | | | | | | | | | ACGGCTCTTTACTATTGTGCGAGAGAT | | GGSYSGTFDH | | | |
| | | | | | | | | | | | | | | | | TTGGGAGGCAGCTACTCGGGGACCTTT | | WGQGTLVTVS | | | |
| | | | | | | | | | | | | | | | | GACCACTGGGGCCAGGGAACCCTGGTC | | S | | | |
| | | | | | | | | | | | | | | | | ACCGTCTCCTCA | | | | | |
| ADI- | IGHV3- | EVQLVQSG | 105 | FTFSNYD | 290 | WVRQP | 480 | AIGTAG | 640 | RLTIS | 891 | TRALFC | 1139 | WGRGTL | 1288 | GAGGTGCAGCTGGTGCAGTCTGGGGGA | 1432 | EVQLVQSGGV | 1715 | | |
| 75715 | 13 | GVLVQPGG | | MH | | PGKRL | | DTYYSD | | RENAR | | GDDCYT | | VTVSS | | GTCTTGGTACAGCCGGGGGGGTCCCTG | | LVQPGGSLRL | | | |
| | | SLRLSCAA | | | | EWVS | | SVKG | | KSLYL | | NWYFDL | | | | AGACTCTCCTGTGCAGCCTCTGGATTC | | SCAASGFTFS | | | |
| | | SG | | | | | | | | QMNSL | | | | | | ACCTTCAGTAACTACGACATGCACTGG | | NYDMHWVRQP | | | |
| | | | | | | | | | | RDGDT | | | | | | GTCCGCCAACCTCCAGGAAAACGTCTG | | PGKRLEWVSA | | | |
| | | | | | | | | | | AVYYC | | | | | | GAATGGGTCTCAGCTATTGGAACTGCT | | IGTAGDTYYS | | | |
| | | | | | | | | | | | | | | | | GGTGACACATACTATTCAGACTCCGTG | | DSVKGRLTIS | | | |
| | | | | | | | | | | | | | | | | AAGGGCCGACTCACCATCTCCAGAGAA | | RENARKSLYL | | | |
| | | | | | | | | | | | | | | | | AATGCCAGGAAGTCCTTGTATCTTCAA | | QMNSLRDGDT | | | |
| | | | | | | | | | | | | | | | | ATGAACAGCCTGAGAGACGGCGACACG | | AVYYCTRALF | | | |
| | | | | | | | | | | | | | | | | GCAGTGTATTACTGTACAAGAGCTCTG | | CGDDCYTNWY | | | |
| | | | | | | | | | | | | | | | | TTTTGTGGTGATGATTGCTACACAAAC | | FDLWGRGTLV | | | |
| | | | | | | | | | | | | | | | | TGGTACTTCGATCTCTGGGGCCGCGGC | | TVSS | | | |
| | | | | | | | | | | | | | | | | ACCCTGGTCACCGTCTCCTCA | | | | | |
| ADI- | IGHV3- | QVTLKESG | 106 | FTFSGFG | 308 | WIRQA | 461 | VISYDG | 641 | RFTIS | 892 | AKDSHD | 1140 | WGQGTT | 1287 | CAGGTCACGTTGAAGGAGTCTGGGGGA | 1433 | QVTLKESGGG | 1716 | | |
| 75854 | 30 | GGVVQPGR | | MH | | PGKGL | | SNKYYE | | RDNSK | | YAMDV | | VTVSS | | GGCGTGGTCCAGCCTGGGAGGTCCCTG | | VVQPGRSLRL | | | |
| | | SLRLSCAA | | | | EWVA | | DSVKG | | NTLYL | | | | | | AGACTCTCCTGTGCAGCCTCTGGTTTC | | SCAASGFTFS | | | |
| | | SG | | | | | | | | QMNSL | | | | | | ACTTTTAGTGGTTTTGGCATGCACTGG | | GFGMHWIRQA | | | |
| | | | | | | | | | | RAEDT | | | | | | ATCCGCCAGGCTCCAGGCAAGGGGCTG | | PGKGLEWVAV | | | |
| | | | | | | | | | | AVYFC | | | | | | GAGTGGGTGGCAGTTATATCATATGAT | | ISYDGSNKYY | | | |
| | | | | | | | | | | | | | | | | GGAAGTAATAAATATTATGAAGACTCC | | EDSVKGRFTI | | | |
| | | | | | | | | | | | | | | | | GTGAAGGGCCGATTCACCATTTCCAGA | | SRDNSKNTLY | | | |
| | | | | | | | | | | | | | | | | GACAATTCCAAGAACACGCTGTATCTG | | LQMNSLRAED | | | |
| | | | | | | | | | | | | | | | | CAAATGAACAGCCTGAGAGCTGAGGAC | | TAVYFCAKDS | | | |
| | | | | | | | | | | | | | | | | ACGGCTGTCTATTTTTGTGCGAAAGAT | | HDYAMDVWGQ | | | |
| | | | | | | | | | | | | | | | | AGTCACGATTACGCTATGGACGTCTGG | | GTTVTVSS | | | |
| | | | | | | | | | | | | | | | | GGCCAAGGGACCACGGTCACCGTCTCC | | | | | |
| | | | | | | | | | | | | | | | | TCA | | | | | |
| ADI- | IGHV2- | QVTLKQSG | 107 | FSIRFNG | 309 | WIRQP | 481 | LIFWDD | 642 | RVSIT | 893 | AHSTDR | 1141 | WGQGTL | 1286 | CAGGTCACGCTGAAGCAGTCTGGTCCT | 1434 | QVTLKQSGPT | 1717 | | |
| 75701 | 5 | PTLVRPTR | | VGVG | | PGKAL | | DKRYSP | | KDTSR | | RNFEWL | | VTVSS | | ACGCTGGTGAGACCCACACGGACCCTC | | LVRPTRTLTV | | | |
| | | TLTVTCTF | | | | EWLG | | SLKS | | NQVVL | | SLSGWG | | | | ACGGTGACGTGCACCTTCTTTGGGTTT | | TCTFFGFSIR | | | |
| | | FG | | | | | | | | TMTNM | | GNFEY | | | | TCAATCAGATTTAATGGAGTGGGTGTG | | FNGVGVGWIR | | | |
| | | | | | | | | | | DPVDT | | | | | | GGCTGGATCCGGCAGCCCCCAGGAAAG | | QPPGKALEWL | | | |
| | | | | | | | | | | ATYYC | | | | | | GCCCTGGAGTGGCTTGGATTAATTTTT | | GLIFWDDDKR | | | |
| | | | | | | | | | | | | | | | | TGGGATGATGATAAGCGGTACAGCCCA | | YSPSLKSRVS | | | |
| | | | | | | | | | | | | | | | | TCTTTGAAGAGCAGGGTCAGCATCACC | | ITKDTSRNQV | | | |
| | | | | | | | | | | | | | | | | AAGGACACCTCCAGAAACCAGGTGGTC | | VLTMTNMDPV | | | |
| | | | | | | | | | | | | | | | | CTTACAATGACCAACATGGACCCTGTG | | DTATYYCAHS | | | |
| | | | | | | | | | | | | | | | | GACACAGCCACATATTACTGTGCACAC | | TDRRNFEWLS | | | |
| | | | | | | | | | | | | | | | | TCCACAGATAGACGCAATTTTGAATGG | | LSGWGGNFEY | | | |
| | | | | | | | | | | | | | | | | TTATCATTGTCCGGGTGGGGGGGGAAC | | WGQGTLVTVS | | | |
| | | | | | | | | | | | | | | | | TTTGAATACTGGGGCCAGGGAACCCTG | | S | | | |
| | | | | | | | | | | | | | | | | GTCACCGTTTCCTCA | | | | | |
| ADI- | IGHV3- | EVQLVQTG | 99 | FIVTRNY | 310 | WVRQA | 437 | LIYPGG | 643 | RFTIS | 777 | ARDRGP | 1142 | WGQGTL | 1286 | GAGGTGCAGCTGGTGCAGACTGGAGGA | 1435 | EVQLVQTGGG | 1718 | | |
| 75681 | 53 | GGLIQPGG | | MS | | PGKGL | | STYYAD | | RDNSK | | SLHDY | | VTVSS | | GGCTTGATCCAGCCGGGGGGGTCCCTG | | LIQPGGSLRL | | | |
| | | SLRLSCAA | | | | EWVS | | SVKG | | NTLYL | | | | | | AGACTCTCCTGTGCAGCCTCTGAATTC | | SCAASEFIVT | | | |
| | | SE | | | | | | | | QMNSL | | | | | | ATCGTCACTAGAAACTACATGAGTTGG | | RNYMSWVRQA | | | |
| | | | | | | | | | | RAEDT | | | | | | GTCCGCCAGGCTCCAGGGAAGGGGCTG | | PGKGLEWVSL | | | |
| | | | | | | | | | | AVYYC | | | | | | GAGTGGGTCTCACTCATATATCCCGGT | | IYPGGSTYYA | | | |
| | | | | | | | | | | | | | | | | GGCAGCACATACTACGCAGACTCCGTG | | DSVKGRFTIS | | | |
| | | | | | | | | | | | | | | | | AAGGGCCGATTCACAATCTCCAGAGAC | | RDNSKNTLYL | | | |
| | | | | | | | | | | | | | | | | AATTCCAAGAACACCCTCTATCTTCAA | | QMNSLRAEDT | | | |
| | | | | | | | | | | | | | | | | ATGAACAGCCTGAGAGCCGAGGACACG | | AVYYCARDRG | | | |
| | | | | | | | | | | | | | | | | GCCGTCTATTACTGTGCGAGAGATAGG | | PSLHDYWGQG | | | |
| | | | | | | | | | | | | | | | | GGGCCTTCACTACACGACTATTGGGGC | | TLVTVSS | | | |
| | | | | | | | | | | | | | | | | CAGGGAACCCTGGTCACCGTCTCCTCA | | | | | |
| ADI- | IGHV3- | EVQLVETG | 80 | IIVTSNY | 311 | WVRQA | 437 | VIYPGG | 644 | RFTVS | 894 | ARERTS | 1143 | WGQGTL | 1286 | GAGGTGCAGCTGGTGGAGACTGGAGGA | 1436 | EVQLVETGGG | 1719 | | |
| 75757 | 53 | GGLIQPGG | | MN | | PGKGL | | TTYYAD | | RDNSK | | GRLD | | VTVSS | | GGCTTGATCCAGCCTGGGGGGTCCCTG | | LIQPGGSLRL | | | |
| | | SLRLSCAA | | | | EWVS | | SVKG | | NTLYL | | | | | | AGACTCTCCTGTGCAGCCTCTGAGATC | | SCAASEIIVT | | | |
| | | SE | | | | | | | | QMNSL | | | | | | ATCGTCACTAGCAACTACATGAACTGG | | SNYMNWVRQA | | | |
| | | | | | | | | | | RAEDT | | | | | | GTCCGCCAGGCTCCAGGGAAGGGGCTG | | PGKGLEWVSV | | | |
| | | | | | | | | | | AVYYC | | | | | | GAGTGGGTCTCAGTCATTTATCCCGGT | | IYPGGTTYYA | | | |
| | | | | | | | | | | | | | | | | GGTACCACATACTACGCAGACTCCGTG | | DSVKGRFTVS | | | |
| | | | | | | | | | | | | | | | | AAGGGCCGATTCACCGTCTCCAGAGAC | | RDNSKNTLYL | | | |
| | | | | | | | | | | | | | | | | AATTCCAAGAACACCCTGTATCTTCAA | | QMNSLRAEDT | | | |
| | | | | | | | | | | | | | | | | ATGAACAGTCTGAGAGCCGAGGACACG | | AVYYCARERT | | | |
| | | | | | | | | | | | | | | | | GCCGTTTATTACTGTGCGAGGGAACGG | | SGRLDWGQGT | | | |
| | | | | | | | | | | | | | | | | ACCAGTGGCCGGCTCGACTGGGGCCAG | | LVTVSS | | | |
| | | | | | | | | | | | | | | | | GGAACCCTGGTCACCGTCTCCTCA | | | | | |
| ADI- | IGHV3- | QVQLVQSG | 108 | FTFSRYW | 312 | WVRQA | 431 | NIKQDG | 645 | RFTIS | 895 | ARDSTP | 1144 | WGQGTQ | 1296 | CAGGTGCAGCTGGTGCAGTCTGGGGGA | 1437 | QVQLVQSGGG | 1720 | | |
| 75656 | 7 | GGLVQPGG | | MT | | PGKGL | | SEKNYV | | RDNAK | | ATYNND | | VTVSS | | GGCTTGGTCCAGCCTGGGGGGTCCATG | | LVQPGGSMRL | | | |
| | | SMRLSCAA | | | | EWVA | | DSVNG | | NSLFL | | WTYPEY | | | | AGACTCTCCTGTGCAGCCTCTGGATTC | | SCAASGFTFS | | | |
| | | ISG | | | | | | | | QMISL | | FDL | | | | ACCTTTAGTCGATATTGGATGACCTGG | | RYWMTWVRQA | | | |
| | | | | | | | | | | RAEDS | | | | | | GTCCGCCAGGCTCCAGGGAAGGGGCTG | | PGKGLEWVAN | | | |
| | | | | | | | | | | AVYFC | | | | | | GAGTGGGTGGCCAACATAAAGCAAGAT | | IKQDGSEKNY | | | |
| | | | | | | | | | | | | | | | | GGAAGTGAGAAAAACTATGTGGACTCT | | VDSVNGRFTI | | | |
| | | | | | | | | | | | | | | | | GTCAACGGCCGATTCACCATCTCCAGA | | SRDNAKNSLF | | | |
| | | | | | | | | | | | | | | | | GACAACGCCAAGAACTCACTGTTTCTG | | LQMISLRAED | | | |
| | | | | | | | | | | | | | | | | CAAATGATCAGCCTGAGAGCCGAGGAC | | SAVYFCARDS | | | |
| | | | | | | | | | | | | | | | | TCGGCTGTGTATTTCTGTGCGAGAGAT | | TPATYNNDWT | | | |
| | | | | | | | | | | | | | | | | TCTACGCCGGCTACTTATAACAATGAC | | YPEYFDLWGQ | | | |
| | | | | | | | | | | | | | | | | TGGACTTACCCTGAATACTTCGACCTC | | GTQVTVSS | | | |
| | | | | | | | | | | | | | | | | TGGGGCCAGGGCACCCAGGTCACCGTC | | | | | |
| | | | | | | | | | | | | | | | | TCCTCA | | | | | |
| ADI- | IGHV3- | QVQLEQSG | 109 | FTFSRYW | 312 | WVRQA | 431 | NIKQDG | 598 | RFTIS | 896 | ARDCTP | 1145 | WGQGTL | 1286 | CAGGTGCAGCTGGAGCAGTCTGGGGGA | 1438 | QVQLEQSGGG | 1721 | | |
| 75677 | 7 | GGLVQPGG | | MT | | PGKGL | | SEKNYV | | RDNAK | | ATYNND | | VTVSS | | GGCTTGGTCCAGCCTGGGGGGTCCTTG | | LVQPGGSLRL | | | |
| | | SLRLSCAA | | | | EWVA | | DSVKG | | NSLFM | | WTYAEY | | | | AGACTCTCCTGTGCAGCCTCTGGATTC | | SCAASGFTFS | | | |
| | | SG | | | | | | | | QMNSL | | FDH | | | | ACCTTTAGTCGGTATTGGATGACCTGG | | RYWMTWVRQA | | | |
| | | | | | | | | | | RAEDT | | | | | | GTCCGCCAGGCTCCAGGGAAGGGGCTG | | PGKGLEWVAN | | | |
| | | | | | | | | | | AVYYC | | | | | | GAGTGGGTGGCCAACATAAAGCAAGAT | | IKQDGSEKNY | | | |
| | | | | | | | | | | | | | | | | GGAAGTGAGAAAAACTATGTGGACTCT | | VDSVKGRFTI | | | |
| | | | | | | | | | | | | | | | | GTGAAGGGCCGATTCACCATCTCCAGA | | SRDNAKNSLF | | | |
| | | | | | | | | | | | | | | | | GACAACGCCAAGAACTCACTGTTTATG | | MQMNSLRAED | | | |
| | | | | | | | | | | | | | | | | CAAATGAACAGCCTGAGAGCCGAGGAC | | TAVYYCARDC | | | |
| | | | | | | | | | | | | | | | | ACGGCTGTGTATTACTGTGCGAGAGAT | | TPATYNNDWT | | | |
| | | | | | | | | | | | | | | | | TGTACGCCGGCTACTTATAACAATGAC | | YAEYFDHWGQ | | | |
| | | | | | | | | | | | | | | | | TGGACATACGCTGAATACTTCGACCAC | | GTLVTVSS | | | |
| | | | | | | | | | | | | | | | | TGGGGCCAGGGCACCCTGGTCACCGTC | | | | | |
| | | | | | | | | | | | | | | | | TCCTCA | | | | | |
| ADI- | IGHV1- | QVQLVQSG | 35 | YSFTSYY | 313 | WVRQA | 433 | IINPSG | 646 | RVTMT | 897 | ARDAGV | 1146 | WGQGTL | 1286 | CAGGTCCAGCTGGTGCAGTCTGGGGCT | 1439 | QVQLVQSGAE | 1722 | | |
| 75860 | 46 | AEVKKPGA | | IQ | | PGQGL | | GSTSYA | | RDEST | | VDPLDP | | VTVSS | | GAGGTGAAGAAGCCTGGGGCCTCAGTG | | VKKPGASVKV | | | |
| | | SVKVSCKA | | | | EWMG | | QKFQG | | RTVYM | | YPLPRF | | | | AAGGTTTCCTGCAAGGCATCTGGATAC | | SCKASGYSFT | | | |
| | | SG | | | | | | | | ELSSL | | | | | | TCCTTCACCAGCTACTATATACAGTGG | | SYYIQWVRQA | | | |
| | | | | | | | | | | RSEDT | | | | | | GTGCGACAGGCCCCTGGACAAGGGCTT | | PGQGLEWMGI | | | |
| | | | | | | | | | | AMYYC | | | | | | GAGTGGATGGGAATAATCAACCCTAGT | | INPSGGSTSY | | | |
| | | | | | | | | | | | | | | | | GGTGGAAGCACAAGCTACGCACAGAAG | | AQKFQGRVTM | | | |
| | | | | | | | | | | | | | | | | TTTCAGGGCAGAGTCACCATGACTAGG | | TRDESTRTVY | | | |
| | | | | | | | | | | | | | | | | GACGAGTCCACGAGAACAGTGTACATG | | MELSSLRSED | | | |
| | | | | | | | | | | | | | | | | GAGCTGAGCAGCCTGAGATCTGAGGAC | | TAMYYCARDA | | | |
| | | | | | | | | | | | | | | | | ACGGCCATGTATTACTGTGCGAGAGAT | | GVVDPLDPYP | | | |
| | | | | | | | | | | | | | | | | GCGGGTGTGGTGGACCCGCTGGACCCC | | LPRFWGQGTL | | | |
| | | | | | | | | | | | | | | | | TACCCCCTGCCTCGTTTCTGGGGCCAG | | VTVSS | | | |
| | | | | | | | | | | | | | | | | GGAACCCTGGTCACCGTCTCCTCA | | | | | |
| ADI- | IGHV3- | QVQLQQSG | 110 | FTFTSYD | 314 | WVRQA | 437 | TIGTTG | 647 | RFTIS | 860 | ARGVQE | 1147 | WGKGTT | 1291 | CAGGTGCAGCTGCAGCAGTCTGGGGGA | 1440 | QVQLQQSGGG | 1723 | | |
| 75672 | 13 | GGLVQPGG | | MH | | PGKGL | | DSYYPG | | RENAK | | ERRGLV | | VTVSS | | GGCTTGGTACAGCCTGGGGGGTCCCTG | | LVQPGGSLRL | | | |
| | | SLRLSCAA | | | | EWVS | | SVKG | | NSLYL | | RKFYYY | | | | AGACTCTCCTGTGCAGCCTCTGGATTC | | SCAASGFTFT | | | |
| | | SG | | | | | | | | QMNSL | | YMDV | | | | ACCTTTACTAGCTACGACATGCACTGG | | SYDMHWVRQA | | | |
| | | | | | | | | | | RAGDT | | | | | | GTCCGCCAAGCTCCAGGAAAGGGTCTG | | PGKGLEWVST | | | |
| | | | | | | | | | | AVYYC | | | | | | GAGTGGGTCTCAACTATTGGAACTACT | | IGTTGDSYYP | | | |
| | | | | | | | | | | | | | | | | GGTGACTCATATTATCCAGGCTCCGTG | | GSVKGRFTIS | | | |
| | | | | | | | | | | | | | | | | AAGGGCCGATTCACCATCTCCAGAGAA | | RENAKNSLYL | | | |
| | | | | | | | | | | | | | | | | AATGCCAAGAACTCCTTGTATCTTCAA | | QMNSLRAGDT | | | |
| | | | | | | | | | | | | | | | | ATGAACAGCCTGAGAGCCGGGGACACG | | AVYYCARGVQ | | | |
| | | | | | | | | | | | | | | | | GCTGTTTATTACTGTGCAAGGGGGGTA | | EERRGLVRKF | | | |
| | | | | | | | | | | | | | | | | CAAGAGGAGCGACGGGGGCTGGTGAGA | | YYYYMDVWGK | | | |
| | | | | | | | | | | | | | | | | AAGTTCTACTACTACTACATGGACGTC | | GTTVTVSS | | | |
| | | | | | | | | | | | | | | | | TGGGGCAAAGGGACCACGGTCACCGTC | | | | | |
| | | | | | | | | | | | | | | | | TCCTCA | | | | | |
| ADI- | IGHV4- | QVQLQESG | 67 | GSISSDH | 315 | WIRQP | 445 | TTYYTA | 648 | RVTIS | 898 | ARVEVS | 1148 | WGQGAL | 1285 | CAGGTGCAGCTGCAGGAGTCGGGCCCG | 1441 | QVQLQESGPG | 1724 | | |
| 75684 | 39 | PGLVKPSE | | FYWG | | PGKGL | | RTNYNP | | VDTSK | | GGFSGY | | VTVSS | | GGACTGGTGAAGCCTTCGGAGACCCTG | | LVKPSETLSL | | | |
| | | TLSLTCTV | | | | EWIG | | SLKS | | NQFSL | | DFLTPY | | | | TCCCTCACCTGCACTGTCTCTGGCGGC | | TCTVSGGSIS | | | |
| | | SG | | | | | | | | KLTSV | | YFDY | | | | TCCATCAGCAGTGATCATTTCTACTGG | | SDHFYWGWIR | | | |
| | | | | | | | | | | TATDT | | | | | | GGCTGGATCCGCCAGCCCCCAGGGAAG | | QPPGKGLEWI | | | |
| | | | | | | | | | | AIYYC | | | | | | GGGCTGGAGTGGATTGGGACCACCTAT | | GTTYYTARTN | | | |
| | | | | | | | | | | | | | | | | TATACTGCCCGCACCAACTACAACCCG | | YNPSLKSRVT | | | |
| | | | | | | | | | | | | | | | | TCCCTCAAGAGTCGGGTCACCATATCC | | ISVDTSKNQF | | | |
| | | | | | | | | | | | | | | | | GTTGACACGTCCAAGAACCAGTTCTCC | | SLKLTSVTAT | | | |
| | | | | | | | | | | | | | | | | CTGAAGCTGACCTCTGTGACCGCCACA | | DTAIYYCARV | | | |
| | | | | | | | | | | | | | | | | GACACGGCTATTTATTACTGTGCGCGA | | EVSGGFSGYD | | | |
| | | | | | | | | | | | | | | | | GTCGAGGTTTCGGGTGGATTTAGTGGC | | FLTPYYFDYW | | | |
| | | | | | | | | | | | | | | | | TACGATTTCCTCACACCGTACTACTTT | | GQGALVTVSS | | | |
| | | | | | | | | | | | | | | | | GACTACTGGGGCCAGGGAGCCCTGGTC | | | | | |
| | | | | | | | | | | | | | | | | ACCGTCTCCTCA | | | | | |
| ADI- | IGHV3- | QVQLQQSG | 111 | FIVSSNY | 260 | WVRQA | 437 | VIYPGG | 649 | RFTIS | 899 | ARDYGD | 1149 | WGQGTL | 1286 | CAGGTCCAGCTGCAGCAGTCTGGAGGA | 1442 | QVQLQQSGGG | 1725 | | |
| 75733 | 53 | GGLIQPGG | | MS | | PGKGL | | STFYAD | | RDTSK | | YYFDY | | VTVSS | | GGCTTGATCCAGCCTGGGGGGTCCCTG | | LIQPGGSLRL | | | |
| | | SLRLSCAA | | | | EWVS | | SVKG | | NTLYL | | | | | | AGACTCTCCTGTGCAGCCTCTGAGTTC | | SCAASEFIVS | | | |
| | | SE | | | | | | | | QMNSL | | | | | | ATCGTCAGTAGCAACTACATGAGCTGG | | SNYMSWVRQA | | | |
| | | | | | | | | | | RAEDT | | | | | | GTCCGCCAGGCTCCAGGGAAGGGGCTG | | PGKGLEWVSV | | | |
| | | | | | | | | | | AVYYC | | | | | | GAGTGGGTCTCAGTTATTTATCCCGGC | | IYPGGSTFYA | | | |
| | | | | | | | | | | | | | | | | GGTAGCACATTCTACGCAGACTCCGTG | | DSVKGRFTIS | | | |
| | | | | | | | | | | | | | | | | AAGGGCCGATTCACCATCTCCAGAGAC | | RDTSKNTLYL | | | |
| | | | | | | | | | | | | | | | | ACTTCCAAGAACACGCTGTATCTTCAA | | QMNSLRAEDT | | | |
| | | | | | | | | | | | | | | | | ATGAACAGCCTGAGAGCCGAGGACACG | | AVYYCARDYG | | | |
| | | | | | | | | | | | | | | | | GCCGTGTATTACTGTGCGAGAGACTAC | | DYYFDYWGQG | | | |
| | | | | | | | | | | | | | | | | GGTGACTACTACTTTGACTACTGGGGC | | TLVTVSS | | | |
| | | | | | | | | | | | | | | | | CAGGGAACCCTGGTCACCGTCTCCTCA | | | | | |
| ADI- | IGHV3- | QVQLVQSG | 112 | FIVSSNY | 260 | WVRQA | 437 | LMYPGG | 650 | RFTIS | 900 | ARVLPL | 1150 | WGQGTL | 1286 | CAGGTGCAGCTGGTGCAGTCTGGGGGA | 1443 | QVQLVQSGGG | 1726 | | |
| 75732 | 66 | GGLVQPGG | | MS | | PGKGL | | STYYAD | | RDKSK | | HGDFFD | | VTVSS | | GGCTTGGTCCAGCCTGGGGGGTCCCTG | | LVQPGGSLRL | | | |
| | | SLRLSCAA | | | | EWVS | | SVKG | | NTLYL | | Y | | | | AGACTCTCCTGTGCAGCCTCTGAATTC | | SCAASEFIVS | | | |
| | | SE | | | | | | | | QMNSL | | | | | | ATCGTCAGTAGTAATTACATGAGCTGG | | SNYMSWVRQA | | | |
| | | | | | | | | | | RAEDT | | | | | | GTCCGCCAGGCTCCAGGGAAGGGGCTG | | PGKGLEWVSL | | | |
| | | | | | | | | | | AVYYC | | | | | | GAGTGGGTCTCACTTATGTATCCCGGT | | MYPGGSTYYA | | | |
| | | | | | | | | | | | | | | | | GGTAGCACATACTACGCAGACTCCGTG | | DSVKGRFTIS | | | |
| | | | | | | | | | | | | | | | | AAGGGCAGATTCACCATCTCCAGAGAC | | RDKSKNTLYL | | | |
| | | | | | | | | | | | | | | | | AAATCCAAGAACACGCTGTATCTTCAA | | QMNSLRAEDT | | | |
| | | | | | | | | | | | | | | | | ATGAACAGCCTGAGAGCCGAGGACACG | | AVYYCARVLP | | | |
| | | | | | | | | | | | | | | | | GCTGTCTATTACTGTGCGAGAGTCCTC | | LHGDFFDYWG | | | |
| | | | | | | | | | | | | | | | | CCTCTCCACGGTGACTTCTTTGACTAC | | QGTLVTVSS | | | |
| | | | | | | | | | | | | | | | | TGGGGCCAGGGAACCCTGGTCACCGTC | | | | | |
| | | | | | | | | | | | | | | | | TCCTCA | | | | | |
| ADI- | IGHV2- | QVTLKESG | 71 | FSFSTGG | 316 | WIRQP | 441 | LIYWDG | 651 | RLTIT | 901 | AHHQIN | 1151 | ISQGTL | 1297 | CAGGTCACGTTGAAGGAGTCTGGTCCT | 1444 | QVTLKESGPT | 1727 | | |
| 75722 | 5 | PTLVKPTQ | | VGVG | | PGKAL | | DKRYSP | | KDTSK | | TIFGV | | VTVSS | | ACGCTGGTGAAACCCACACAGACCCTC | | LVKPTQTLTL | | | |
| | | TLTLTCTF | | | | EWLA | | SLKN | | NQVVL | | | | | | ACGCTGACCTGCACCTTCTCTGGGTTC | | TCTFSGFSFS | | | |
| | | SG | | | | | | | | TVTNM | | | | | | TCATTCAGCACTGGTGGAGTGGGTGTG | | TGGVGVGWIR | | | |
| | | | | | | | | | | DPVDT | | | | | | GGCTGGATCCGTCAGCCCCCCGGAAAG | | QPPGKALEWL | | | |
| | | | | | | | | | | ATYYC | | | | | | GCCCTAGAGTGGCTTGCACTCATTTAT | | ALIYWDGDKR | | | |
| | | | | | | | | | | | | | | | | TGGGATGGTGATAAGCGCTACAGCCCA | | YSPSLKNRLT | | | |
| | | | | | | | | | | | | | | | | TCTCTGAAGAACAGGCTCACCATCACC | | ITKDTSKNQV | | | |
| | | | | | | | | | | | | | | | | AAGGACACCTCCAAAAACCAGGTGGTC | | VLTVTNMDPV | | | |
| | | | | | | | | | | | | | | | | CTTACAGTGACCAACATGGACCCTGTG | | DTATYYCAHH | | | |
| | | | | | | | | | | | | | | | | GACACAGCCACATATTACTGTGCACAC | | QINTIFGVIS | | | |
| | | | | | | | | | | | | | | | | CACCAAATCAATACGATTTTTGGAGTA | | QGTLVTVSS | | | |
| | | | | | | | | | | | | | | | | ATTAGCCAGGGAACCCTGGTCACCGTC | | | | | |
| | | | | | | | | | | | | | | | | TCCTCA | | | | | |
| ADI- | IGHV2- | QITLKESG | 113 | FSLSTTG | 271 | WIRQP | 441 | LIYWDD | 652 | RLSIT | 853 | AHTPDG | 1152 | WGQGTL | 1286 | CAGATCACGTTGAAGGAGTCTGGTCCT | 1445 | QITLKESGPT | 1728 | | |
| 75674 | 5 | PTLVKPTQ | | VGVG | | PGKAL | | DQRYSP | | KDTSK | | RYFEWL | | VTVSS | | ACCCTGGTGAAACCCACACAGACCCTC | | LVKPTQTLTL | | | |
| | | TLTLTCTF | | | | EWLA | | SLQS | | NQVVL | | TPLFGN | | | | ACGCTGACCTGCACCTTCTCTGGTTTC | | TCTFSGFSLS | | | |
| | | SG | | | | | | | | TMTNM | | YYFDY | | | | TCACTCAGCACTACTGGAGTGGGTGTG | | TTGVGVGWIR | | | |
| | | | | | | | | | | DPVDT | | | | | | GGCTGGATCCGTCAGCCCCCAGGAAAG | | QPPGKALEWL | | | |
| | | | | | | | | | | ATYYC | | | | | | GCCCTGGAGTGGCTTGCACTCATTTAT | | ALIYWDDDQR | | | |
| | | | | | | | | | | | | | | | | TGGGATGATGATCAGCGCTACAGCCCA | | YSPSLQSRLS | | | |
| | | | | | | | | | | | | | | | | TCTCTGCAGAGCAGGCTCAGCATCACC | | ITKDTSKNQV | | | |
| | | | | | | | | | | | | | | | | AAGGACACCTCCAAAAACCAGGTGGTC | | VLTMTNMDPV | | | |
| | | | | | | | | | | | | | | | | CTTACAATGACCAACATGGACCCTGTG | | DTATYYCAHT | | | |
| | | | | | | | | | | | | | | | | GACACAGCCACATATTACTGTGCACAC | | PDGRYFEWLT | | | |
| | | | | | | | | | | | | | | | | ACCCCGGACGGGCGATATTTTGAGTGG | | PLFGNYYFDY | | | |
| | | | | | | | | | | | | | | | | TTAACGCCTCTTTTTGGTAACTACTAC | | WGQGTLVTVS | | | |
| | | | | | | | | | | | | | | | | TTTGACTACTGGGGCCAGGGAACCCTG | | S | | | |
| | | | | | | | | | | | | | | | | GTCACCGTCTCCTCA | | | | | |
| ADI- | IGHV3- | QVQLVQSG | 114 | IIVVRNY | 317 | WVRQA | 437 | VIYSGG | 653 | RFTIS | 902 | ARGRPS | 1153 | WGQGTL | 1286 | CAGGTGCAGCTGGTGCAGTCTGGGGGA | 1446 | QVQLVQSGGA | 1729 | | |
| 75638 | 66 | GALVQPGG | | MS | | PGKGL | | TTFYAD | | RDKSK | | GLYDY | | VTVSS | | GCCTTGGTCCAGCCGGGGGGGTCCCTG | | LVQPGGSLRL | | | |
| | | SLRLACAA | | | | EWVS | | SVKG | | NTLYL | | | | | | AGACTCGCCTGTGCAGCCTCTGAAATC | | ACAASEIIVV | | | |
| | | SE | | | | | | | | QMSSL | | | | | | ATCGTCGTTAGGAACTACATGAGCTGG | | RNYMSWVRQA | | | |
| | | | | | | | | | | RGEDT | | | | | | GTCCGCCAGGCTCCAGGGAAGGGGCTG | | PGKGLEWVSV | | | |
| | | | | | | | | | | AVYYC | | | | | | GAGTGGGTCTCAGTTATTTACAGCGGA | | IYSGGTTFYA | | | |
| | | | | | | | | | | | | | | | | GGTACCACATTCTACGCAGACTCCGTG | | DSVKGRFTIS | | | |
| | | | | | | | | | | | | | | | | AAGGGCCGGTTCACCATCTCCAGAGAC | | RDKSKNTLYL | | | |
| | | | | | | | | | | | | | | | | AAATCCAAGAACACGCTGTATCTTCAA | | QMSSLRGEDT | | | |
| | | | | | | | | | | | | | | | | ATGAGCAGCCTGAGAGGTGAGGACACG | | AVYYCARGRP | | | |
| | | | | | | | | | | | | | | | | GCTGTGTATTACTGTGCGAGAGGGCGA | | SGLYDYWGQG | | | |
| | | | | | | | | | | | | | | | | CCATCCGGATTATATGACTACTGGGGC | | TLVTVSS | | | |
| | | | | | | | | | | | | | | | | CAGGGAACCCTGGTCACCGTCTCCTCA | | | | | |
| ADI- | IGHV1- | QVQLVQSG | 115 | YTFSDYY | 318 | WVRQA | 433 | MINPDA | 654 | RVTMT | 903 | ARDIAG | 1154 | WGQGTL | 1286 | CAGGTGCAGCTGGTACAGTCTGGGGCT | 1447 | QVQLVQSGAE | 1730 | | |
| 75800 | 46 | AEVKKPGA | | IH | | PGQGL | | ASIAYA | | SDTST | | IPATLG | | VTVSS | | GAGGTGAAGAAGCCTGGGGCCTCAGTG | | VKKPGASVKI | | | |
| | | SVKISCKA | | | | EWMG | | QKFQG | | STIYM | | LDP | | | | AAGATTTCCTGCAAGGCATCTGGATAC | | SCKASGYTFS | | | |
| | | SG | | | | | | | | ELSSL | | | | | | ACTTTCAGCGACTACTATATTCACTGG | | DYYIHWVRQA | | | |
| | | | | | | | | | | RSEDT | | | | | | GTGCGACAGGCCCCTGGACAAGGGCTT | | PGQGLEWMGM | | | |
| | | | | | | | | | | AVYYC | | | | | | GAGTGGATGGGAATGATCAACCCTGAT | | INPDAASIAY | | | |
| | | | | | | | | | | | | | | | | GCTGCTAGCATTGCTTACGCACAGAAG | | AQKFQGRVTM | | | |
| | | | | | | | | | | | | | | | | TTCCAGGGCAGAGTTACCATGACGAGT | | TSDTSTSTIY | | | |
| | | | | | | | | | | | | | | | | GACACGTCCACGAGCACAATTTATATG | | MELSSLRSED | | | |
| | | | | | | | | | | | | | | | | GAACTGAGTAGCCTGAGATCTGAGGAC | | TAVYYCARDI | | | |
| | | | | | | | | | | | | | | | | ACGGCCGTGTATTATTGTGCGAGAGAT | | AGIPATLGLD | | | |
| | | | | | | | | | | | | | | | | ATCGCAGGAATACCAGCTACTTTAGGC | | PWGQGTLVTV | | | |
| | | | | | | | | | | | | | | | | CTCGACCCCTGGGGCCAGGGAACCCTG | | SS | | | |
| | | | | | | | | | | | | | | | | GTCACCGTCTCCTCA | | | | | |
| ADI- | IGHV4- | QVTLKESG | 116 | GSFSSGY | 319 | WIRQP | 445 | EISQSG | 655 | RLSIS | 904 | ARGRGY | 1155 | WGQGAL | 1285 | CAGGTCACCTTGAAGGAGTCGGGCGCG | 1448 | QVTLKESGAG | 1731 | | |
| 75675 | 34 | AGLLKPSE | | YWN | | PGKGL | | GANSNP | | IETAK | | GSGSDS | | VTVSS | | GGACTGTTGAAGCCCTCGGAGACCCTG | | LLKPSETLSL | | | |
| | | TLSLTCAV | | | | EWIG | | SLKG | | KQFSL | | FDY | | | | TCCCTCACCTGCGCTGTCTCTGGTGGG | | TCAVSGGSFS | | | |
| | | SG | | | | | | | | KLRSV | | | | | | TCCTTCAGCAGTGGTTATTACTGGAAC | | SGYYWNWIRQ | | | |
| | | | | | | | | | | TAADT | | | | | | TGGATCCGCCAGCCCCCAGGCAAGGGG | | PPGKGLEWIG | | | |
| | | | | | | | | | | ALYYC | | | | | | CTGGAGTGGATTGGAGAAATCAGTCAG | | EISQSGGANS | | | |
| | | | | | | | | | | | | | | | | AGTGGTGGAGCCAACTCCAACCCGTCC | | NPSLKGRLSI | | | |
| | | | | | | | | | | | | | | | | CTCAAGGGTCGACTCTCCATATCAATA | | SIETAKKQFS | | | |
| | | | | | | | | | | | | | | | | GAGACGGCCAAGAAGCAGTTCTCCCTG | | LKLRSVTAAD | | | |
| | | | | | | | | | | | | | | | | AAGCTGAGGTCTGTGACCGCCGCGGAC | | TALYYCARGR | | | |
| | | | | | | | | | | | | | | | | ACGGCTCTCTATTACTGTGCGAGAGGC | | GYGSGSDSFD | | | |
| | | | | | | | | | | | | | | | | CGCGGTTACGGATCAGGGAGTGACTCC | | YWGQGALVTV | | | |
| | | | | | | | | | | | | | | | | TTTGACTATTGGGGCCAGGGAGCCCTG | | SS | | | |
| | | | | | | | | | | | | | | | | GTCACCGTCTCCTCA | | | | | |
| ADI- | IGHV4- | QVQLQQSG | 117 | DSISSYY | 320 | WIRQP | 445 | DIYHSG | 656 | RVTIS | 905 | ARGPKE | 1156 | WGQGTL | 1286 | CAGGTGCAGCTGCAGCAGTCGGGCCCA | 1449 | QVQLQQSGPG | 1732 | | |
| 75818 | 59 | PGLVKPSE | | WS | | PGKGL | | STNYNP | | VDTSK | | RWFNNW | | VTVSS | | GGACTGGTGAAGCCTTCGGAGACCCTG | | LVKPSETLSL | | | |
| | | TLSLTCIV | | | | EWIG | | SLKS | | SQFSL | | FDP | | | | TCCCTCACCTGCATTGTCTCTGGTGAC | | TCIVSGDSIS | | | |
| | | SG | | | | | | | | KLRSV | | | | | | TCCATCAGTAGTTACTATTGGAGCTGG | | SYYWSWIRQP | | | |
| | | | | | | | | | | TAADT | | | | | | ATCCGGCAGCCCCCAGGGAAGGGACTG | | PGKGLEWIGD | | | |
| | | | | | | | | | | AVYYC | | | | | | GAGTGGATTGGGGACATCTATCACAGT | | IYHSGSTNYN | | | |
| | | | | | | | | | | | | | | | | GGGAGCACCAACTACAATCCCTCCCTC | | PSLKSRVTIS | | | |
| | | | | | | | | | | | | | | | | AAGAGTCGAGTCACCATATCAGTAGAC | | VDTSKSQFSL | | | |
| | | | | | | | | | | | | | | | | ACGTCCAAGAGTCAGTTCTCCTTGAAG | | KLRSVTAADT | | | |
| | | | | | | | | | | | | | | | | CTGAGGTCTGTGACCGCTGCGGACACG | | AVYYCARGPK | | | |
| | | | | | | | | | | | | | | | | GCCGTCTATTACTGTGCGAGAGGCCCT | | ERWFNNWFDP | | | |
| | | | | | | | | | | | | | | | | AAAGAGCGCTGGTTCAACAATTGGTTC | | WGQGTLVTVS | | | |
| | | | | | | | | | | | | | | | | GACCCCTGGGGCCAAGGAACCCTGGTC | | S | | | |
| | | | | | | | | | | | | | | | | ACCGTCTCCTCA | | | | | |
| ADI- | IGHV1- | QVQLVQSG | 118 | GTFSSYN | 321 | WVRQA | 433 | RIIPIL | 657 | RVTIT | 906 | AREGGS | 1157 | WGQGTL | 1286 | CAGGTGCAGCTGGTGCAGTCTGGGGCT | 1450 | QVQLVQSGAE | 1733 | | |
| 75741 | 69 | AEVKKPGS | | VN | | PGQGL | | GIANYA | | ADRST | | TTEYFP | | VTVSS | | GAGGTGAAGAAGCCTGGGTCCTCGGTG | | VKKPGSSVKV | | | |
| | | SVKVSCK- | | | | EWMG | | EKFQD | | STAYM | | H | | | | AAGGTCTCCTGCAAGGGCACCTTCAGC | | SCKGTFSSYN | | | |
| | | -- | | | | | | | | ELSSL | | | | | | AGTTATAATGTCAACTGGGTGCGACAG | | VNWVRQAPGQ | | | |
| | | | | | | | | | | RYDDT | | | | | | GCCCCTGGACAAGGGCTTGAGTGGATG | | GLEWMGRIIP | | | |
| | | | | | | | | | | AVYYC | | | | | | GGAAGGATCATCCCTATCCTTGGTATT | | ILGIANYAEK | | | |
| | | | | | | | | | | | | | | | | GCAAACTACGCAGAGAAGTTCCAGGAC | | FQDRVTITAD | | | |
| | | | | | | | | | | | | | | | | AGAGTCACCATTACCGCGGACAGATCC | | RSTSTAYMEL | | | |
| | | | | | | | | | | | | | | | | ACGAGCACAGCCTACATGGAGCTGAGC | | SSLRYDDTAV | | | |
| | | | | | | | | | | | | | | | | AGCCTGAGATATGACGACACGGCCGTA | | YYCAREGGST | | | |
| | | | | | | | | | | | | | | | | TATTACTGTGCGAGAGAAGGTGGTAGC | | TEYFPHWGQG | | | |
| | | | | | | | | | | | | | | | | ACCACTGAATACTTCCCACACTGGGGC | | TLVTVSS | | | |
| | | | | | | | | | | | | | | | | CAGGGCACCCTGGTCACCGTCTCCTCA | | | | | |
| ADI- | IGHV3- | QVQLQESG | 119 | FTDISNY | 322 | WVRQA | 437 | VIYSGG | 658 | RFTIS | 907 | ARGQVP | 1158 | WGQGTT | 1287 | CAGGTGCAGCTGCAGGAGTCCGGAGGA | 1451 | QVQLQESGGD | 1734 | | |
| 75795 | 53 | GDLVQPGG | | MS | | PGKGL | | TAYYAD | | RHNSK | | NDDYVM | | VTVSS | | GACTTGGTCCAGCCTGGGGGGTCCCTG | | LVQPGGSLRL | | | |
| | | SLRLSCAA | | | | EWVS | | SVKG | | NTLYL | | SGAFDI | | | | AGACTCTCTTGTGCAGCCTCTAGGTTC | | SCAASRFTDI | | | |
| | | SR | | | | | | | | QMNSL | | | | | | ACCGACATTAGCAACTACATGAGCTGG | | SNYMSWVRQA | | | |
| | | | | | | | | | | RTEDT | | | | | | GTCCGCCAGGCTCCAGGGAAGGGGCTG | | PGKGLEWVSV | | | |
| | | | | | | | | | | AVYYC | | | | | | GAGTGGGTCTCAGTTATTTATAGTGGT | | IYSGGTAYYA | | | |
| | | | | | | | | | | | | | | | | GGAACCGCATATTACGCAGACTCCGTG | | DSVKGRFTIS | | | |
| | | | | | | | | | | | | | | | | AAGGGCCGATTCACCATCTCCAGACAC | | RHNSKNTLYL | | | |
| | | | | | | | | | | | | | | | | AACTCCAAGAACACACTGTATCTTCAA | | QMNSLRTEDT | | | |
| | | | | | | | | | | | | | | | | ATGAACAGCCTGAGAACTGAGGACACG | | AVYYCARGQV | | | |
| | | | | | | | | | | | | | | | | GCCGTGTATTACTGTGCGAGAGGCCAA | | PNDDYVMSGA | | | |
| | | | | | | | | | | | | | | | | GTCCCCAACGATGACTACGTTATGAGT | | FDIWGQGTTV | | | |
| | | | | | | | | | | | | | | | | GGTGCTTTTGATATCTGGGGCCAAGGG | | TVSS | | | |
| | | | | | | | | | | | | | | | | ACCACGGTCACCGTCTCCTCA | | | | | |
| ADI- | IGHV3- | QVQLVQTG | 120 | VTVTNNY | 323 | WVRQA | 437 | VIYSGG | 659 | RFTIS | 908 | ARDLME | 1159 | WGQGTT | 1287 | CAGGTGCAGCTGGTGCAGACTGGAGGA | 1452 | QVQLVQTGGG | 1735 | | |
| 75816 | 53 | GGLIQPGG | | MN | | PGKGL | | STYYAD | | RDNSK | | AGGMDV | | VTVSS | | GGCTTGATCCAGCCTGGGGGGTCCCTG | | LIQPGGSLRL | | | |
| | | SLRLSCVA | | | | EWVS | | SVKG | | NTLYL | | | | | | AGACTCTCCTGTGTAGCCTCTGGGGTC | | SCVASGVTVI | | | |
| | | SG | | | | | | | | QMNSL | | | | | | ACCGTCACTAACAACTACATGAACTGG | | NNYMNWVRQA | | | |
| | | | | | | | | | | RADDT | | | | | | GTCCGCCAGGCTCCAGGGAAGGGGCTG | | PGKGLEWVSV | | | |
| | | | | | | | | | | AVYYC | | | | | | GAGTGGGTCTCAGTTATTTATAGTGGT | | IYSGGSTYYA | | | |
| | | | | | | | | | | | | | | | | GGCAGCACATACTACGCAGACTCCGTG | | DSVKGRFTIS | | | |
| | | | | | | | | | | | | | | | | AAGGGCCGATTCACCATCTCCAGAGAC | | RDNSKNTLYL | | | |
| | | | | | | | | | | | | | | | | AATTCCAAGAACACGCTCTATCTTCAA | | QMNSLRADDT | | | |
| | | | | | | | | | | | | | | | | ATGAACAGCCTGAGAGCCGACGACACG | | AVYYCARDLM | | | |
| | | | | | | | | | | | | | | | | GCCGTATATTACTGTGCGAGAGATTTG | | EAGGMDVWGQ | | | |
| | | | | | | | | | | | | | | | | ATGGAGGCGGGCGGTATGGACGTCTGG | | GTTVTVSS | | | |
| | | | | | | | | | | | | | | | | GGCCAAGGGACCACGGTCACCGTCTCC | | | | | |
| | | | | | | | | | | | | | | | | TCA | | | | | |
| ADI- | IGHV1- | QVQLQQSG | 86 | GTFSSYS | 324 | WVRQA | 433 | RIFPIL | 660 | RVTIT | 909 | AREGGK | 1160 | WGQGTL | 1286 | CAGGTACAGCTGCAGCAGTCTGGGGCT | 1453 | QVQLQQSGAE | 1736 | | |
| 75725 | 69 | AEVKKPGS | | VS | | PGQGL | | GVADFA | | ADRST | | TTEFFP | | VTVSS | | GAGGTGAAGAAGCCTGGGTCCTCGGTG | | VKKPGSSVKV | | | |
| | | SVKVSCKA | | | | EWMG | | QKFQD | | STAYM | | L | | | | AAGGTCTCCTGCAAGGCCTCTGGAGGC | | SCKASGGTFS | | | |
| | | SG | | | | | | | | EMSRL | | | | | | ACCTTCAGCAGTTATAGTGTCAGCTGG | | SYSVSWVRQA | | | |
| | | | | | | | | | | RPEDT | | | | | | GTGCGTCAGGCCCCTGGACAAGGGCTT | | PGQGLEWMGR | | | |
| | | | | | | | | | | AVYYC | | | | | | GAGTGGATGGGAAGGATCTTCCCTATC | | IFPILGVADF | | | |
| | | | | | | | | | | | | | | | | CTTGGTGTTGCAGACTTCGCACAGAAG | | AQKFQDRVTI | | | |
| | | | | | | | | | | | | | | | | TTCCAGGACAGAGTCACGATTACCGCG | | TADRSTSTAY | | | |
| | | | | | | | | | | | | | | | | GACAGATCCACGAGCACAGCCTACATG | | MEMSRLRPED | | | |
| | | | | | | | | | | | | | | | | GAGATGAGTAGGTTAAGGCCTGAGGAC | | TAVYYCAREG | | | |
| | | | | | | | | | | | | | | | | ACGGCCGTCTATTACTGTGCGAGGGAA | | GKTTEFFPLW | | | |
| | | | | | | | | | | | | | | | | GGTGGTAAGACCACTGAATTCTTCCCC | | GQGTLVTVSS | | | |
| | | | | | | | | | | | | | | | | CTCTGGGGCCAGGGCACCCTGGTCACC | | | | | |
| | | | | | | | | | | | | | | | | GTCTCCTCA | | | | | |
| ADI- | IGHV3- | QVQLVESG | 24 | FTFNTYW | 325 | WVRQA | 431 | SIKQDG | 661 | RFNIS | 910 | AREMGI | 1161 | RGQGSL | 1298 | CAGGTGCAGCTGGTGGAGTCTGGGGGA | 1454 | QVQLVESGGG | 1737 | | |
| 75686 | 7 | GGLVQPGG | | MS | | PGKGL | | GEKYYV | | RDNAK | | FWFDP | | VTVSS | | GGCTTGGTCCAGCCTGGGGGGTCCCTG | | LVQPGGSLRL | | | |
| | | SLRLSCAA | | | | EWVA | | DSVKG | | NSLFL | | | | | | AGACTCTCCTGTGCAGCCTCTGGATTC | | SCAASGFTFN | | | |
| | | SG | | | | | | | | QMSSL | | | | | | ACCTTTAATACGTATTGGATGAGTTGG | | TYWMSWVRQA | | | |
| | | | | | | | | | | RAEDT | | | | | | GTCCGCCAGGCTCCAGGGAAGGGGCTG | | PGKGLEWVAS | | | |
| | | | | | | | | | | AVYYC | | | | | | GAGTGGGTGGCCAGCATAAAACAAGAT | | IKQDGGEKYY | | | |
| | | | | | | | | | | | | | | | | GGAGGTGAGAAATACTATGTGGACTCT | | VDSVKGRFNI | | | |
| | | | | | | | | | | | | | | | | GTGAAGGGCCGATTTAACATCTCCAGA | | SRDNAKNSLF | | | |
| | | | | | | | | | | | | | | | | GACAACGCCAAGAACTCACTGTTTCTG | | LQMSSLRAED | | | |
| | | | | | | | | | | | | | | | | CAAATGAGCAGCCTGAGAGCCGAGGAC | | TAVYYCAREM | | | |
| | | | | | | | | | | | | | | | | ACGGCTGTCTATTACTGTGCGAGAGAA | | GIFWFDPRGQ | | | |
| | | | | | | | | | | | | | | | | ATGGGTATATTCTGGTTCGACCCCCGG | | GSLVTVSS | | | |
| | | | | | | | | | | | | | | | | GGCCAAGGATCCCTGGTCACCGTCTCC | | | | | |
| | | | | | | | | | | | | | | | | TCA | | | | | |
| ADI- | IGHV1- | RYTLQQSG | 121 | GTFNSYS | 326 | WVRQA | 482 | RIIPIL | 662 | RVNII | 911 | ARDVGY | 1162 | WGQGAL | 1285 | AGGTACACGCTGCAGCAGTCTGGGGCT | 1455 | RYTLQQSGAE | 1738 | | |
| 75717 | 69 | AEVKKPGS | | IS | | PEQGL | | GVTDYP | | ADKST | | SGSGST | | VTVSS | | GAGGTGAAGAAGCCTGGGTCTTCGGTG | | VKKPGSSVKV | | | |
| | | SVKVSCKA | | | | EWMG | | QKFQG | | GTAYM | | YYFDL | | | | AAGGTCTCTTGCAAGGCCTCTGGAGGC | | SCKASGGTFN | | | |
| | | SG | | | | | | | | ELSSL | | | | | | ACCTTCAACAGCTATAGTATCAGTTGG | | SYSISWVRQA | | | |
| | | | | | | | | | | RSEDT | | | | | | GTGCGACAGGCCCCTGAGCAAGGGCTT | | PEQGLEWMGR | | | |
| | | | | | | | | | | AVYYC | | | | | | GAGTGGATGGGAAGGATCATCCCTATC | | IIPILGVTDY | | | |
| | | | | | | | | | | | | | | | | CTTGGTGTAACAGACTACCCACAGAAG | | PQKFQGRVNI | | | |
| | | | | | | | | | | | | | | | | TTCCAGGGCAGAGTCAACATTATCGCG | | IADKSTGTAY | | | |
| | | | | | | | | | | | | | | | | GACAAGTCCACGGGCACAGCTTACATG | | MELSSLRSED | | | |
| | | | | | | | | | | | | | | | | GAGCTGAGCAGCCTGAGATCTGAAGAC | | TAVYYCARDV | | | |
| | | | | | | | | | | | | | | | | ACGGCCGTGTATTACTGCGCGAGAGAT | | GYSGSGSTYY | | | |
| | | | | | | | | | | | | | | | | GTGGGTTATTCTGGCTCGGGGAGTACT | | FDLWGQGALV | | | |
| | | | | | | | | | | | | | | | | TACTACTTTGACCTCTGGGGCCAGGGA | | TVSS | | | |
| | | | | | | | | | | | | | | | | GCCCTGGTCACCGTCTCCTCA | | | | | |
| ADI- | IGHV3- | QVTLKESG | 122 | FTFDEYA | 327 | WVRQV | 436 | GSSWNS | 663 | RFTIS | 912 | VKLRTV | 1163 | WGQGTL | 1286 | CAGGTCACGCTGAAGGAGTCTGGGGGA | 1456 | QVTLKESGGG | 1739 | | |
| 75771 | 9 | GGLVQPGR | | IH | | PGKGL | | GIIGYA | | RDNAK | | TAFDFW | | VTVSS | | GGCTTGGTACAGCCTGGCAGGTCCCTG | | LVQPGRSLRL | | | |
| | | SLRLSCAA | | | | EWVS | | DSVKG | | NSLYL | | SGSRPS | | | | AGACTCTCCTGTGCAGCCTCTGGATTC | | SCAASGFTFD | | | |
| | | SG | | | | | | | | QMNSL | | PKQDFD | | | | ACCTTTGATGAATATGCCATTCACTGG | | EYAIHWVRQV | | | |
| | | | | | | | | | | RPEDT | | Y | | | | GTCCGGCAAGTTCCGGGGAAGGGCCTG | | PGKGLEWVSG | | | |
| | | | | | | | | | | ALYYC | | | | | | GAGTGGGTCTCAGGTTCCAGTTGGAAT | | SSWNSGIIGY | | | |
| | | | | | | | | | | | | | | | | AGTGGTATCATAGGCTATGCGGACTCT | | ADSVKGRFTI | | | |
| | | | | | | | | | | | | | | | | GTGAAGGGCCGATTCACCATCTCCAGA | | SRDNAKNSLY | | | |
| | | | | | | | | | | | | | | | | GACAACGCCAAGAACTCCCTGTATCTG | | LQMNSLRPED | | | |
| | | | | | | | | | | | | | | | | CAAATGAACAGTCTGAGACCTGAGGAC | | TALYYCVKLR | | | |
| | | | | | | | | | | | | | | | | ACGGCCTTGTATTACTGTGTAAAACTC | | TVTAFDFWSG | | | |
| | | | | | | | | | | | | | | | | AGAACGGTCACGGCTTTCGATTTTTGG | | SRPSPKQDFD | | | |
| | | | | | | | | | | | | | | | | AGTGGTTCCCGCCCGAGCCCCAAGCAA | | YWGQGTLVTV | | | |
| | | | | | | | | | | | | | | | | GACTTTGACTACTGGGGCCAGGGAACC | | SS | | | |
| | | | | | | | | | | | | | | | | CTGGTCACCGTCTCCTCA | | | | | |
| ADI- | IGHV2- | RSTLEQSG | 123 | FSLSTSG | 328 | WIRQS | 483 | LIDWDD | 664 | RLTIS | 913 | TRTGAI | 1164 | WGQGTT | 1287 | AGGTCCACGCTGGAGCAGTCTGGTCCT | 1457 | RSTLEQSGPA | 1740 | | |
| 75651 | 70 | PALVKPTQ | | TCVN | | PGKAL | | DKYYST | | KDTSK | | IAARPW | | VTVSS | | GCGCTGGTGAAACCCACACAGACCCTC | | LVKPTQTLRL | | | |
| | | TLRLTCTF | | | | EWLA | | SLKT | | NQVVL | | YNYGMD | | | | AGACTGACCTGCACCTTCTCTGGGTTC | | TCTFSGFSLS | | | |
| | | SG | | | | | | | | TMTNM | | V | | | | TCACTCAGCACTAGTGGAACGTGTGTG | | TSGTCVNWIR | | | |
| | | | | | | | | | | DPVDT | | | | | | AACTGGATCCGTCAGTCCCCAGGGAAG | | QSPGKALEWL | | | |
| | | | | | | | | | | ATYYC | | | | | | GCCCTGGAGTGGCTTGCACTCATTGAT | | ALIDWDDDKY | | | |
| | | | | | | | | | | | | | | | | TGGGACGATGATAAATACTACAGCACA | | YSTSLKTRLT | | | |
| | | | | | | | | | | | | | | | | TCTCTGAAGACCAGGCTCACCATCTCC | | ISKDTSKNQV | | | |
| | | | | | | | | | | | | | | | | AAGGACACCTCCAAAAACCAGGTGGTC | | VLTMTNMDPV | | | |
| | | | | | | | | | | | | | | | | CTTACAATGACCAACATGGACCCTGTT | | DTATYYCTRT | | | |
| | | | | | | | | | | | | | | | | GACACAGCCACGTATTATTGTACACGG | | GAIIAARPWY | | | |
| | | | | | | | | | | | | | | | | ACGGGGGCGATTATAGCAGCTCGTCCT | | NYGMDVWGQG | | | |
| | | | | | | | | | | | | | | | | TGGTACAACTACGGTATGGACGTCTGG | | TTVTVSS | | | |
| | | | | | | | | | | | | | | | | GGCCAAGGGACCACGGTCACCGTCTCC | | | | | |
| | | | | | | | | | | | | | | | | TCA | | | | | |
| ADI- | IGHV5- | QVQLVQSG | 124 | YSFTTYW | 329 | WVRQM | 476 | TIYPGD | 665 | QVTIS | 914 | ASQRQR | 1165 | WGKGTT | 1291 | CAGGTCCAGCTGGTGCAGTCTGGAGCA | 1458 | QVQLVQSGAE | 1741 | | |
| 75813 | 51 | AEVKKPGE | | IA | | PGKGL | | SDTRYS | | ADKSI | | GFYYYM | | VTVSS | | GAGGTGAAAAAGCCCGGGGAGTCTCTG | | VKKPGESLKI | | | |
| | | SLKISCEG | | | | EWMG | | PSFQG | | STAYL | | DV | | | | AAGATCTCCTGTGAGGGTTCTGGATAC | | SCEGSGYSFT | | | |
| | | SG | | | | | | | | QWSSL | | | | | | AGCTTTACCACCTACTGGATCGCCTGG | | TYWIAWVRQM | | | |
| | | | | | | | | | | KASDT | | | | | | GTGCGCCAGATGCCCGGGAAAGGCCTG | | PGKGLEWMGT | | | |
| | | | | | | | | | | AIYYC | | | | | | GAGTGGATGGGGACCATCTATCCTGGT | | IYPGDSDTRY | | | |
| | | | | | | | | | | | | | | | | GACTCTGATACCAGATACAGCCCGTCC | | SPSFQGQVTI | | | |
| | | | | | | | | | | | | | | | | TTCCAAGGCCAGGTCACCATCTCAGCC | | SADKSISTAY | | | |
| | | | | | | | | | | | | | | | | GACAAGTCCATCAGTACCGCCTACTTG | | LQWSSLKASD | | | |
| | | | | | | | | | | | | | | | | CAGTGGAGCAGCCTGAAGGCCTCAGAC | | TAIYYCASQR | | | |
| | | | | | | | | | | | | | | | | ACCGCCATTTATTACTGTGCGAGTCAG | | QRGFYYYMDV | | | |
| | | | | | | | | | | | | | | | | CGTCAGCGTGGCTTCTACTACTACATG | | WGKGTTVTVS | | | |
| | | | | | | | | | | | | | | | | GACGTCTGGGGCAAAGGGACCACGGTC | | S | | | |
| | | | | | | | | | | | | | | | | ACCGTCTCCTCA | | | | | |
| ADI- | IGHV3- | QVQLVQSG | 10 | FIFSNYG | 330 | WVRQA | 431 | VISYDG | 666 | RFTIS | 915 | AKMTSS | 1166 | WGQGTT | 1287 | CAGGTGCAGCTGGTGCAGTCTGGGGGA | 1459 | QVQLVQSGGG | 1742 | | |
| 75750 | 30 | GGVVQPGR | | MH | | PGKGL | | SHKYYV | | RDNSK | | SPILYY | | VTVSS | | GGCGTGGTCCAGCCTGGGAGGTCCCTG | | VVQPGRSLRL | | | |
| | | SLRLSCAA | | | | EWVA | | DSVEG | | NTLYL | | YHGMDV | | | | AGACTCTCCTGTGCAGCCTCTGGATTC | | SCAASGFIFS | | | |
| | | SG | | | | | | | | QMDTL | | | | | | ATCTTCAGTAATTATGGCATGCACTGG | | NYGMHWVRQA | | | |
| | | | | | | | | | | RAEDT | | | | | | GTCCGCCAGGCTCCAGGCAAGGGGCTG | | PGKGLEWVAV | | | |
| | | | | | | | | | | AVYYC | | | | | | GAGTGGGTGGCAGTTATATCATATGAT | | ISYDGSHKYY | | | |
| | | | | | | | | | | | | | | | | GGAAGTCATAAATACTATGTAGACTCC | | VDSVEGRFTI | | | |
| | | | | | | | | | | | | | | | | GTTGAGGGCCGATTCACCATCTCCAGA | | SRDNSKNTLY | | | |
| | | | | | | | | | | | | | | | | GACAATTCCAAGAACACGCTGTATCTG | | LQMDTLRAED | | | |
| | | | | | | | | | | | | | | | | CAAATGGACACCTTGAGAGCTGAGGAC | | TAVYYCAKMT | | | |
| | | | | | | | | | | | | | | | | ACGGCTGTGTATTACTGTGCGAAAATG | | SSSPILYYYH | | | |
| | | | | | | | | | | | | | | | | ACTAGCAGCTCGCCCATTCTCTACTAC | | GMDVWGQGTT | | | |
| | | | | | | | | | | | | | | | | TACCACGGTATGGACGTCTGGGGCCAA | | VTVSS | | | |
| | | | | | | | | | | | | | | | | GGGACCACGGTCACCGTCTCCTCA | | | | | |
| ADI- | IGHV3- | RCPLVQSG | 125 | FTFSRYD | 331 | WVRQV | 484 | AIGTAG | 667 | RFTIS | 916 | ARAEFS | 1167 | WGRGTL | 1288 | AGGTGCCCGCTGGTGCAGTCTGGGGGA | 1460 | RCPLVQSGGG | 1743 | | |
| 75608 | 13 | GGLVQPGG | | MH | | TGRGL | | DTYYPD | | RENAK | | GYVLHF | | VTVSS | | GGCTTGGTACAGCCTGGGGGGTCCCTG | | LVQPGGSLRL | | | |
| | | SLRLSCAA | | | | EWVS | | SVKG | | NSLYL | | DL | | | | AGACTCTCCTGTGCAGCCTCTGGATTC | | SCAASGFTFS | | | |
| | | SG | | | | | | | | QMNSL | | | | | | ACCTTCAGTAGGTACGACATGCACTGG | | RYDMHWVRQV | | | |
| | | | | | | | | | | RAGDT | | | | | | GTCCGCCAAGTTACGGGAAGAGGTCTG | | TGRGLEWVSA | | | |
| | | | | | | | | | | AIYYC | | | | | | GAGTGGGTCTCAGCGATTGGTACTGCT | | IGTAGDTYYP | | | |
| | | | | | | | | | | | | | | | | GGTGACACATATTATCCAGACTCCGTG | | DSVKGRFTIS | | | |
| | | | | | | | | | | | | | | | | AAGGGCCGATTCACCATCTCCAGAGAA | | RENAKNSLYL | | | |
| | | | | | | | | | | | | | | | | AATGCCAAGAACTCATTGTATCTTCAA | | QMNSLRAGDT | | | |
| | | | | | | | | | | | | | | | | ATGAACAGCCTGAGAGCCGGGGACACG | | AIYYCARAEF | | | |
| | | | | | | | | | | | | | | | | GCTATTTATTACTGTGCAAGAGCTGAA | | SGYVLHFDLW | | | |
| | | | | | | | | | | | | | | | | TTTAGTGGCTACGTTCTCCACTTCGAT | | GRGTLVTVSS | | | |
| | | | | | | | | | | | | | | | | CTCTGGGGCCGTGGCACCCTGGTCACC | | | | | |
| | | | | | | | | | | | | | | | | GTCTCCTCA | | | | | |
| ADI- | IGHV3- | QVQLVQSG | 12 | FTFSSYW | 332 | WVRQA | 431 | SIKQDG | 668 | RFNIS | 917 | AREMGI | 1161 | WGQGSL | 1289 | CAGGTGCAGCTGGTGCAGTCTGGGGGA | 1461 | QVQLVQSGGG | 1744 | | |
| 75663 | 7 | GGLVQPGG | | MS | | PGKGL | | SEQYYV | | RDNAK | | FWFDP | | VTVSS | | GGCTTGGTCCAGCCTGGGGGGTCCCTG | | LVQPGGSLRL | | | |
| | | SLRLSCAA | | | | EWVA | | DSVKG | | NSLFL | | | | | | AGACTCTCCTGTGCAGCCTCTGGATTC | | SCAASGFTFS | | | |
| | | SG | | | | | | | | QMNSL | | | | | | ACCTTTAGTAGCTATTGGATGAGCTGG | | SYWMSWVRQA | | | |
| | | | | | | | | | | RAEDT | | | | | | GTCCGCCAGGCTCCAGGGAAGGGGCTG | | PGKGLEWVAS | | | |
| | | | | | | | | | | AVYYC | | | | | | GAGTGGGTGGCCAGCATAAAGCAAGAT | | IKQDGSEQYY | | | |
| | | | | | | | | | | | | | | | | GGAAGTGAGCAATACTATGTGGACTCT | | VDSVKGRFNI | | | |
| | | | | | | | | | | | | | | | | GTGAAGGGCCGATTTAACATCTCCAGA | | SRDNAKNSLF | | | |
| | | | | | | | | | | | | | | | | GACAACGCCAAGAACTCACTGTTTCTG | | LQMNSLRAED | | | |
| | | | | | | | | | | | | | | | | CAAATGAACAGCCTGAGAGCCGAGGAC | | TAVYYCAREM | | | |
| | | | | | | | | | | | | | | | | ACGGCTGTCTATTACTGTGCGAGAGAA | | GIFWFDPWGQ | | | |
| | | | | | | | | | | | | | | | | ATGGGTATATTTTGGTTCGACCCCTGG | | GSLVTVSS | | | |
| | | | | | | | | | | | | | | | | GGCCAGGGATCCCTGGTCACCGTCTCC | | | | | |
| | | | | | | | | | | | | | | | | TCA | | | | | |
| ADI- | IGHV3- | RCPLVQSG | 126 | FTVSSNY | 281 | WVRQA | 485 | LIYPGG | 643 | RFTVS | 894 | AREIYG | 1168 | WGQGTL | 1286 | AGGTGCCCGCTGGTGCAGTCTGGGGGA | 1462 | RCPLVQSGGD | 1745 | | |
| 75652 | 66 | GDLVQPGG | | MS | | PGKGL | | STYYAD | | RDNSK | | CTTCSH | | VTVSS | | GACTTGGTCCAGCCTGGGGGGTCCCTC | | LVQPGGSLRL | | | |
| | | SLRLSCEA | | | | QWVS | | SVKG | | NTLYL | | DIPRDS | | | | AGACTCTCCTGTGAAGCCTCTGGATTC | | SCEASGFTVS | | | |
| | | SG | | | | | | | | QMNSL | | | | | | ACCGTCAGTAGCAACTACATGAGCTGG | | SNYMSWVRQA | | | |
| | | | | | | | | | | RAEDT | | | | | | GTCCGCCAGGCTCCAGGGAAGGGGCTG | | PGKGLQWVSL | | | |
| | | | | | | | | | | AVYYC | | | | | | CAGTGGGTTTCACTCATTTATCCCGGT | | IYPGGSTYYA | | | |
| | | | | | | | | | | | | | | | | GGTAGCACATACTACGCAGACTCCGTG | | DSVKGRFTVS | | | |
| | | | | | | | | | | | | | | | | AAGGGCCGATTCACCGTCTCCAGAGAC | | RDNSKNTLYL | | | |
| | | | | | | | | | | | | | | | | AATTCCAAGAACACGCTGTATCTTCAA | | QMNSLRAEDT | | | |
| | | | | | | | | | | | | | | | | ATGAACAGCCTGAGAGCTGAGGACACG | | AVYYCAREIY | | | |
| | | | | | | | | | | | | | | | | GCTGTCTATTACTGTGCGAGAGAAATC | | GCTTCSHDIP | | | |
| | | | | | | | | | | | | | | | | TACGGCTGTACCACCTGCTCTCACGAC | | RDSWGQGTLV | | | |
| | | | | | | | | | | | | | | | | ATCCCACGGGACTCCTGGGGCCAGGGA | | TVSS | | | |
| | | | | | | | | | | | | | | | | ACCCTGGTCACCGTCTCCTCA | | | | | |
| ADI- | IGHV3- | QVQLQQSG | 116 | FTFSNYW | 333 | WVRQA | 486 | RINRDG | 669 | RFTIS | 918 | ARDLES | 1169 | WGQGTL | 1286 | CAGGTGCAGCTGCAGCAGTCTGGGGGA | 1463 | QVQLQQSGGG | 1746 | | |
| 75567 | 74 | GGLVQPGG | | MH | | PGKGL | | SSPTYA | | RDNAK | | GSFHPG | | VTVSS | | GGCTTAGTTCAGCCTGGGGGGTCCCTG | | LVQPGGSLRL | | | |
| | | SLRLSCAA | | | | VWVS | | DSVKG | | NTLFL | | LVRVP | | | | AGACTCTCCTGTGCAGCCTCTGGATTC | | SCAASGFTFS | | | |
| | | SG | | | | | | | | QMNSL | | | | | | ACCTTCAGTAACTACTGGATGCACTGG | | NYWMHWVRQA | | | |
| | | | | | | | | | | RAEDT | | | | | | GTCCGCCAAGCTCCAGGGAAGGGGCTG | | PGKGLVWVSR | | | |
| | | | | | | | | | | AVYYC | | | | | | GTGTGGGTCTCACGTATTAATAGAGAT | | INRDGSSPTY | | | |
| | | | | | | | | | | | | | | | | GGGAGTAGTCCAACTTACGCGGACTCC | | ADSVKGRFTI | | | |
| | | | | | | | | | | | | | | | | GTGAAGGGCCGATTCACCATCTCCAGA | | SRDNAKNTLF | | | |
| | | | | | | | | | | | | | | | | GACAACGCCAAGAACACGCTGTTTCTG | | LQMNSLRAED | | | |
| | | | | | | | | | | | | | | | | CAAATGAACAGTCTGAGAGCCGAAGAC | | TAVYYCARDL | | | |
| | | | | | | | | | | | | | | | | ACGGCTGTGTATTACTGTGCAAGAGAC | | ESGSFHPGLV | | | |
| | | | | | | | | | | | | | | | | CTGGAAAGTGGGAGCTTTCATCCCGGC | | RVPWGQGTLV | | | |
| | | | | | | | | | | | | | | | | CTGGTTCGGGTCCCCTGGGGCCAGGGA | | TVSS | | | |
| | | | | | | | | | | | | | | | | ACCCTGGTCACCGTCTCCTCA | | | | | |
| ADI- | IGHV4- | QVQLQQWG | 127 | GSFSEYY | 334 | WVRQS | 438 | KITHSG | 670 | RVTIS | 919 | ARHVPE | 1170 | WGQGSL | 1289 | CAGGTGCAGCTACAGCAGTGGGGCGCA | 1464 | QVQLQQWGAG | 1747 | | |
| 75670 | 34 | AGLLKPSE | | WT | | PGKGL | | NTDYNP | | VDTSK | | DVVVEP | | VTVSS | | GGACTGTTGAAGCCTTCGGAGACCCTG | | LLKPSETLSL | | | |
| | | TLSLTCGV | | | | EWIG | | SLKS | | NQFSL | | PAPWGL | | | | TCCCTCACCTGCGGTGTCTATGGTGGG | | TCGVYGGSFS | | | |
| | | YG | | | | | | | | RLSSV | | DY | | | | TCCTTCAGTGAATACTACTGGACCTGG | | EYYWTWVRQS | | | |
| | | | | | | | | | | TAADS | | | | | | GTCCGCCAGTCCCCAGGGAAGGGGCTT | | PGKGLEWIGK | | | |
| | | | | | | | | | | AVYYC | | | | | | GAGTGGATTGGTAAAATCACTCATAGT | | ITHSGNTDYN | | | |
| | | | | | | | | | | | | | | | | GGAAACACCGACTACAACCCGTCCCTC | | PSLKSRVTIS | | | |
| | | | | | | | | | | | | | | | | AAGAGTCGAGTCACCATATCAGTGGAC | | VDTSKNQFSL | | | |
| | | | | | | | | | | | | | | | | ACGTCCAAGAACCAGTTCTCCCTGAGG | | RLSSVTAADS | | | |
| | | | | | | | | | | | | | | | | TTGAGCTCTGTGACCGCCGCGGACTCG | | AVYYCARHVP | | | |
| | | | | | | | | | | | | | | | | GCTGTGTATTACTGTGCGAGACATGTT | | EDVVVEPPAP | | | |
| | | | | | | | | | | | | | | | | CCGGAGGATGTTGTAGTAGAACCACCT | | WGLDYWGQGS | | | |
| | | | | | | | | | | | | | | | | GCCCCGTGGGGACTTGACTACTGGGGC | | LVTVSS | | | |
| | | | | | | | | | | | | | | | | CAGGGATCCCTGGTCACCGTCTCCTCA | | | | | |
| ADI- | IGHV3- | QVQLVQSG | 12 | FTFSNYW | 333 | WVRQA | 486 | RINRDG | 669 | RFTIS | 918 | ARDLES | 1169 | WGQGTL | 1286 | CAGGTGCAGCTGGTGCAGTCTGGGGGA | 1465 | QVQLVQSGGG | 1748 | | |
| 75602 | 74 | GGLVQPGG | | MH | | PGKGL | | SSPTYA | | RDNAK | | GSFHPG | | VTVSS | | GGCTTAGTTCAGCCTGGGGGGTCCCTG | | LVQPGGSLRL | | | |
| | | SLRLSCAA | | | | VWVS | | DSVKG | | NTLFL | | LVRVP | | | | AGACTCTCCTGTGCAGCCTCTGGATTC | | SCAASGFTFS | | | |
| | | SG | | | | | | | | QMNSL | | | | | | ACCTTCAGTAACTACTGGATGCACTGG | | NYWMHWVRQA | | | |
| | | | | | | | | | | RAEDT | | | | | | GTCCGCCAAGCTCCAGGGAAGGGGCTG | | PGKGLVWVSR | | | |
| | | | | | | | | | | AVYYC | | | | | | GTGTGGGTCTCACGTATTAATAGAGAT | | INRDGSSPTY | | | |
| | | | | | | | | | | | | | | | | GGGAGTAGTCCAACTTACGCGGACTCC | | ADSVKGRFTI | | | |
| | | | | | | | | | | | | | | | | GTGAAGGGCCGATTCACCATCTCCAGA | | SRDNAKNTLF | | | |
| | | | | | | | | | | | | | | | | GACAACGCCAAGAACACGCTGTTTCTG | | LQMNSLRAED | | | |
| | | | | | | | | | | | | | | | | CAAATGAACAGTCTGAGAGCCGAAGAC | | TAVYYCARDL | | | |
| | | | | | | | | | | | | | | | | ACGGCTGTGTATTACTGTGCAAGAGAC | | ESGSFHPGLV | | | |
| | | | | | | | | | | | | | | | | CTGGAAAGTGGGAGCTTTCATCCCGGC | | RVPWGQGTLV | | | |
| | | | | | | | | | | | | | | | | CTGGTTCGGGTCCCCTGGGGCCAGGGA | | TVSS | | | |
| | | | | | | | | | | | | | | | | ACCCTGGTCACCGTCTCCTCA | | | | | |
| ADI- | IGHV3- | EVQLVQTG | 99 | IIVSRNY | 291 | WVRQA | 437 | VIYPGG | 622 | RFTIS | 920 | AREDNN | 1123 | WGQGTT | 1287 | GAGGTGCAGCTGGTGCAGACTGGAGGA | 1466 | EVQLVQTGGG | 1749 | | |
| 75774 | 53 | GGLIQPGG | | MT | | PGKGL | | STFYAD | | RDDSK | | NAFDI | | VTVSS | | GGCTTGATCCAGCCGGGGGGGTCCCTG | | LIQPGGSLRL | | | |
| | | SLRLSCAA | | | | EWVS | | SVRG | | NTLYL | | | | | | AGACTCTCCTGTGCAGCCTCTGAGATC | | SCAASEIIVS | | | |
| | | SE | | | | | | | | QMTSL | | | | | | ATCGTCAGTAGGAACTACATGACCTGG | | RNYMTWVRQA | | | |
| | | | | | | | | | | RAEDT | | | | | | GTCCGCCAGGCTCCAGGGAAAGGGCTG | | PGKGLEWVSV | | | |
| | | | | | | | | | | AVYYC | | | | | | GAGTGGGTCTCAGTTATTTATCCCGGT | | IYPGGSTFYA | | | |
| | | | | | | | | | | | | | | | | GGGAGCACTTTCTACGCAGACTCCGTG | | DSVRGRFTIS | | | |
| | | | | | | | | | | | | | | | | AGGGGCCGATTCACCATCTCCAGAGAC | | RDDSKNTLYL | | | |
| | | | | | | | | | | | | | | | | GATTCCAAGAACACTCTGTATCTTCAA | | QMTSLRAEDT | | | |
| | | | | | | | | | | | | | | | | ATGACCAGCCTGAGAGCCGAGGACACG | | AVYYCAREDN | | | |
| | | | | | | | | | | | | | | | | GCCGTGTATTACTGTGCGAGAGAAGAC | | NNAFDIWGQG | | | |
| | | | | | | | | | | | | | | | | AATAATAATGCTTTTGATATTTGGGGG | | TTVTVSS | | | |
| | | | | | | | | | | | | | | | | CAAGGGACAACGGTCACCGTCTCCTCA | | | | | |
| ADI- | IGHV1- | QVQLQQSG | 128 | YTFSDYY | 335 | WVRQA | 433 | ILNPSA | 671 | RVTMT | 921 | ARDWRF | 1171 | WGQGTT | 1287 | CAGGTGCAGCTACAGCAGTCTGGGGCT | 1467 | QVQLQQSGAE | 1750 | | |
| 75736 | 46 | AEVKKPGA | | MH | | PGQGL | | GSTSYA | | SDTST | | LPGFDA | | VTVSS | | GAGGTGAAGAAGCCTGGGGCCTCAGTG | | VKKPGASVRV | | | |
| | | SVRVSCKA | | | | EWMG | | QKFQG | | NTVYM | | FDI | | | | AGGGTTTCCTGCAAGGCATCTGGATAC | | SCKASGYTFS | | | |
| | | SG | | | | | | | | ELTSL | | | | | | ACCTTCAGTGACTACTATATGCACTGG | | DYYMHWVRQA | | | |
| | | | | | | | | | | RSEDT | | | | | | GTGCGACAGGCCCCTGGACAAGGGCTT | | PGQGLEWMGI | | | |
| | | | | | | | | | | AVYYC | | | | | | GAGTGGATGGGAATACTCAACCCTAGT | | LNPSAGSTSY | | | |
| | | | | | | | | | | | | | | | | GCTGGTAGCACAAGCTACGCACAGAAG | | AQKFQGRVTM | | | |
| | | | | | | | | | | | | | | | | TTCCAGGGCAGAGTCACCATGACCAGT | | TSDTSTNTVY | | | |
| | | | | | | | | | | | | | | | | GACACGTCCACGAACACAGTCTACATG | | MELTSLRSED | | | |
| | | | | | | | | | | | | | | | | GAGCTGACCAGCCTGAGATCTGAGGAC | | TAVYYCARDW | | | |
| | | | | | | | | | | | | | | | | ACGGCCGTGTATTACTGTGCTAGAGAT | | RFLPGFDAFD | | | |
| | | | | | | | | | | | | | | | | TGGCGGTTTCTACCCGGATTTGATGCT | | IWGQGTTVTV | | | |
| | | | | | | | | | | | | | | | | TTTGATATCTGGGGCCAAGGGACCACG | | SS | | | |
| | | | | | | | | | | | | | | | | GTCACCGTCTCCTCA | | | | | |
| ADI- | IGHV3- | QVQLVESG | 129 | FTFDDYA | 336 | WVRQA | 437 | GVSWNS | 672 | RFIIS | 922 | AKFGGG | 1172 | WGQGTL | 1286 | CAGGTGCAGCTGGTGGAGTCTGGGGGA | 1468 | QVQLVESGGA | 1751 | | |
| 75586 | 9 | GALVQPGR | | IH | | PGKGL | | GIIGYA | | RDNAK | | PTNYDF | | VTVSS | | GCCTTGGTACAGCCTGGCAGGTCCCTG | | LVQPGRSLRL | | | |
| | | SLRLSCAA | | | | EWVS | | DSVKG | | NSLYL | | RSASPY | | | | AGACTCTCCTGTGCAGCCTCTGGATTC | | SCAASGFTFD | | | |
| | | SG | | | | | | | | HMNSL | | HFDS | | | | ACCTTTGATGATTATGCCATCCACTGG | | DYAIHWVRQA | | | |
| | | | | | | | | | | RPEDT | | | | | | GTCCGGCAAGCTCCAGGGAAGGGCCTG | | PGKGLEWVSG | | | |
| | | | | | | | | | | ALYYC | | | | | | GAGTGGGTCTCAGGTGTTAGTTGGAAT | | VSWNSGIIGY | | | |
| | | | | | | | | | | | | | | | | AGTGGTATTATAGGCTATGCGGACTCT | | ADSVKGRFII | | | |
| | | | | | | | | | | | | | | | | GTGAAGGGCCGATTCATCATCTCCAGA | | SRDNAKNSLY | | | |
| | | | | | | | | | | | | | | | | GACAACGCCAAGAACTCCCTGTATCTG | | LHMNSLRPED | | | |
| | | | | | | | | | | | | | | | | CATATGAACAGTCTGAGACCTGAGGAC | | TALYYCAKFG | | | |
| | | | | | | | | | | | | | | | | ACGGCCTTGTATTACTGTGCAAAATTT | | GGPTNYDFRS | | | |
| | | | | | | | | | | | | | | | | GGGGGCGGGCCCACAAATTACGATTTT | | ASPYHFDSWG | | | |
| | | | | | | | | | | | | | | | | CGGAGTGCTTCCCCGTATCACTTTGAC | | QGTLVTVSS | | | |
| | | | | | | | | | | | | | | | | TCCTGGGGCCAGGGAACCCTGGTCACC | | | | | |
| | | | | | | | | | | | | | | | | GTCTCCTCA | | | | | |
| ADI- | IGHV3- | QVQLVQSG | 12 | FTFSTYW | 337 | WVRQA | 431 | SIKQDG | 661 | RFNIS | 917 | AREMGI | 1161 | RGQGSL | 1298 | CAGGTGCAGCTGGTGCAGTCTGGGGGG | 1469 | QVQLVQSGGG | 1752 | | |
| 75687 | 7 | GGLVQPGG | | MS | | PGKGL | | GEKYYV | | RDNAK | | FWFDP | | VTVSS | | GGCTTGGTCCAGCCTGGGGGGTCCCTG | | LVQPGGSLRL | | | |
| | | SLRLSCAA | | | | EWVA | | DSVKG | | NSLFL | | | | | | AGACTCTCCTGTGCAGCCTCTGGATTC | | SCAASGFTFS | | | |
| | | SG | | | | | | | | QMNSL | | | | | | ACCTTTAGTACCTATTGGATGAGTTGG | | TYWMSWVRQA | | | |
| | | | | | | | | | | RAEDT | | | | | | GTCCGCCAGGCTCCAGGGAAGGGGCTG | | PGKGLEWVAS | | | |
| | | | | | | | | | | AVYYC | | | | | | GAGTGGGTGGCCAGCATAAAGCAAGAT | | IKQDGGEKYY | | | |
| | | | | | | | | | | | | | | | | GGAGGTGAAAAATACTATGTGGACTCT | | VDSVKGRFNI | | | |
| | | | | | | | | | | | | | | | | GTGAAGGGCCGATTTAACATCTCCAGA | | SRDNAKNSLF | | | |
| | | | | | | | | | | | | | | | | GACAACGCCAAGAACTCACTGTTTCTG | | LQMNSLRAED | | | |
| | | | | | | | | | | | | | | | | CAAATGAACAGCCTGAGAGCCGAGGAC | | TAVYYCAREM | | | |
| | | | | | | | | | | | | | | | | ACGGCTGTCTATTATTGTGCGAGAGAA | | GIFWFDPRGQ | | | |
| | | | | | | | | | | | | | | | | ATGGGTATATTCTGGTTCGACCCCCGG | | GSLVTVSS | | | |
| | | | | | | | | | | | | | | | | GGCCAGGGATCCCTGGTCACCGTCTCC | | | | | |
| | | | | | | | | | | | | | | | | TCA | | | | | |
| ADI- | IGHV4- | QVQLQESG | 130 | YSISSGY | 338 | WIRQP | 487 | SIYHSG | 673 | RVTIS | 923 | ARDGQG | 1173 | WGQGAL | 1285 | CAGGTGCAGCTGCAGGAGTCGGGCCCA | 1470 | QVQLQESGPG | 1753 | | |
| 75694 | 38-2 | PGLVKASE | | SWG | | LGKGL | | STYYKA | | LDTSK | | YVPPRD | | VTVSS | | GGACTGGTGAAGGCTTCGGAGACCCTG | | LVKASETLSL | | | |
| | | TLSLACTV | | | | EWIG | | SLKS | | NQFSL | | NFDH | | | | TCCCTCGCCTGCACTGTCTCTGGTTAC | | ACTVSGYSIS | | | |
| | | SG | | | | | | | | KLSSV | | | | | | TCCATCAGCAGTGGTTATTCCTGGGGC | | SGYSWGWIRQ | | | |
| | | | | | | | | | | TAADT | | | | | | TGGATCCGGCAGCCCCTAGGGAAGGGG | | PLGKGLEWIG | | | |
| | | | | | | | | | | AVYYC | | | | | | CTGGAGTGGATTGGGAGTATCTATCAT | | SIYHSGSTYY | | | |
| | | | | | | | | | | | | | | | | AGTGGGAGCACCTATTACAAAGCGTCC | | KASLKSRVTI | | | |
| | | | | | | | | | | | | | | | | CTCAAGAGTCGAGTCACCATATCACTA | | SLDTSKNQFS | | | |
| | | | | | | | | | | | | | | | | GACACGTCCAAGAACCAGTTCTCCCTG | | LKLSSVTAAD | | | |
| | | | | | | | | | | | | | | | | AAGCTGAGCTCTGTGACGGCCGCAGAC | | TAVYYCARDG | | | |
| | | | | | | | | | | | | | | | | ACGGCCGTCTATTACTGTGCGAGAGAT | | QGYVPPRDNF | | | |
| | | | | | | | | | | | | | | | | GGTCAAGGGTACGTACCACCGAGAGAC | | DHWGQGALVT | | | |
| | | | | | | | | | | | | | | | | AACTTTGACCACTGGGGCCAGGGAGCC | | VSS | | | |
| | | | | | | | | | | | | | | | | CTGGTCACCGTCTCCTCA | | | | | |
| ADI- | IGHV3- | QVQLVQSG | 12 | ITVSSNY | 339 | WVRQA | 437 | VLYAGG | 674 | RFTIS | 924 | ARDIQR | 1174 | WGQGTT | 1287 | CAGGTGCAGCTGGTGCAGTCTGGAGGA | 1471 | QVQLVQSGGG | 1754 | | |
| 75711 | 53 | GGLVQPGG | | MT | | PGKGL | | STFYTD | | RHNSN | | YGIDV | | VTVSS | | GGCTTGGTCCAGCCGGGGGGGTCCCTG | | LVQPGGSLRL | | | |
| | | SLRLSCAA | | | | EWVS | | SVKG | | NTLYL | | | | | | AGACTCTCCTGTGCAGCCTCTGGAATC | | SCAASGITVS | | | |
| | | SG | | | | | | | | QMNSL | | | | | | ACCGTCAGTAGCAACTACATGACCTGG | | SNYMTWVRQA | | | |
| | | | | | | | | | | RPEDT | | | | | | GTCCGCCAGGCTCCAGGGAAGGGGCTG | | PGKGLEWVSV | | | |
| | | | | | | | | | | AVYYC | | | | | | GAGTGGGTCTCAGTTCTTTATGCCGGT | | LYAGGSTFYT | | | |
| | | | | | | | | | | | | | | | | GGTAGCACCTTCTACACAGACTCCGTG | | DSVKGRFTIS | | | |
| | | | | | | | | | | | | | | | | AAGGGCCGGTTCACCATCTCCAGACAC | | RHNSNNTLYL | | | |
| | | | | | | | | | | | | | | | | AATTCCAACAACACACTGTATCTTCAA | | QMNSLRPEDT | | | |
| | | | | | | | | | | | | | | | | ATGAACAGCCTGAGACCTGAAGACACG | | AVYYCARDIQ | | | |
| | | | | | | | | | | | | | | | | GCCGTTTATTATTGTGCGAGAGACATT | | RYGIDVWGQG | | | |
| | | | | | | | | | | | | | | | | CAGCGATACGGTATAGACGTCTGGGGC | | TTVTVSS | | | |
| | | | | | | | | | | | | | | | | CAAGGGACCACGGTCACCGTCTCCTCA | | | | | |
| ADI- | IGHV4- | QVQLQESG | 39 | GSISSDG | 340 | WIRQH | 450 | YIYYSG | 675 | SFTIS | 925 | ARVVRY | 1175 | WGQGTL | 1286 | CAGGTGCAGCTGCAGGAGTCGGGCCCA | 1472 | QVQLQESGPG | 1755 | | |
| 75704 | 31 | PGLVKPSQ | | YYWS | | PGKGL | | ITYYNP | | VDTSK | | CSSTSC | | VTVSS | | GGACTGGTGAAGCCTTCACAGACCCTG | | LVKPSQTLSL | | | |
| | | TLSLTCTV | | | | EWIG | | SLKS | | NQFSL | | YTLDF | | | | TCCCTCACCTGCACTGTCTCTGGTGGC | | TCTVSGGSIS | | | |
| | | SG | | | | | | | | KLSSV | | | | | | TCCATCAGCAGTGATGGTTACTACTGG | | SDGYYWSWIR | | | |
| | | | | | | | | | | TAADT | | | | | | AGCTGGATCCGCCAGCACCCGGGGAAG | | QHPGKGLEWI | | | |
| | | | | | | | | | | AMYYC | | | | | | GGCCTGGAGTGGATTGGGTACATCTAT | | GYIYYSGITY | | | |
| | | | | | | | | | | | | | | | | TACAGTGGGATCACCTACTACAACCCG | | YNPSLKSSFT | | | |
| | | | | | | | | | | | | | | | | TCCCTCAAGAGTTCATTTACCATATCA | | ISVDTSKNQF | | | |
| | | | | | | | | | | | | | | | | GTAGACACGTCTAAGAACCAGTTCTCC | | SLKLSSVTAA | | | |
| | | | | | | | | | | | | | | | | CTGAAGCTGAGCTCTGTGACTGCCGCG | | DTAMYYCARV | | | |
| | | | | | | | | | | | | | | | | GACACGGCCATGTATTACTGTGCGAGA | | VRYCSSTSCY | | | |
| | | | | | | | | | | | | | | | | GTAGTGAGATACTGTAGTAGTACCAGC | | TLDFWGQGTL | | | |
| | | | | | | | | | | | | | | | | TGCTATACGCTTGACTTCTGGGGCCAG | | VTVSS | | | |
| | | | | | | | | | | | | | | | | GGAACCCTGGTCACCGTCTCCTCA | | | | | |
| ADI- | IGHV3- | RCTLVQSG | 131 | FSFSSFA | 341 | WVRQA | 437 | IISGSG | 676 | RFTIS | 777 | AKGPWC | 1176 | WGQGTL | 1286 | AGGTGCACGCTGGTGCAGTCTGGGGGA | 1473 | RCTLVQSGGG | 1756 | | |
| 75631 | 23 | GGLVQPGG | | MN | | PGKGL | | VNTYYA | | RDNSK | | STSNCY | | VTVSS | | GGCTTGGTACAGCCGGGGGGGTCCCTG | | LVQPGGSLRL | | | |
| | | SLRLSCSA | | | | EWVS | | DSVKG | | NTLYL | | TSMVYP | | | | AGACTCTCCTGTTCAGCCTCTGGATTC | | SCSASGFSFS | | | |
| | | SG | | | | | | | | QMNSL | | VADFFH | | | | AGCTTTAGCAGCTTTGCCATGAACTGG | | SFAMNWVRQA | | | |
| | | | | | | | | | | RAEDT | | H | | | | GTCCGCCAGGCTCCAGGGAAGGGGCTG | | PGKGLEWVSI | | | |
| | | | | | | | | | | AVYYC | | | | | | GAGTGGGTCTCAATAATTAGCGGTAGT | | ISGSGVNTYY | | | |
| | | | | | | | | | | | | | | | | GGTGTTAACACCTACTACGCAGACTCC | | ADSVKGRFTI | | | |
| | | | | | | | | | | | | | | | | GTGAAGGGCCGGTTCACCATCTCCAGA | | SRDNSKNTLY | | | |
| | | | | | | | | | | | | | | | | GACAATTCCAAGAACACGCTGTATCTG | | LQMNSLRAED | | | |
| | | | | | | | | | | | | | | | | CAAATGAACAGCCTGAGAGCCGAGGAC | | TAVYYCAKGP | | | |
| | | | | | | | | | | | | | | | | ACGGCCGTATATTACTGTGCGAAAGGT | | WCSTSNCYTS | | | |
| | | | | | | | | | | | | | | | | CCCTGGTGTAGTACTAGCAACTGCTAT | | MVYPVADFFH | | | |
| | | | | | | | | | | | | | | | | ACTTCAATGGTCTATCCCGTCGCTGAT | | HWGQGTLVTV | | | |
| | | | | | | | | | | | | | | | | TTCTTCCACCACTGGGGCCAGGGCACC | | SS | | | |
| | | | | | | | | | | | | | | | | CTGGTCACCGTCTCCTCA | | | | | |
| ADI- | IGHV1- | QVQLVQSG | 50 | GTFNNHF | 342 | WVRRA | 488 | KIIPII | 677 | RVTIT | 926 | ARRTSS | 1177 | WGQGTL | 1286 | CAGGTGCAGCTGGTGCAGTCTGGGGCT | 1474 | QVQLVQSGAE | 1757 | | |
| 75773 | 69 | AEVKKPGS | | IN | | PGQGL | | DIANSA | | ADKST | | SSEYFD | | VTVSS | | GAGGTGAAGAAGCCTGGGTCCTCGGTG | | VKKPGSSVKV | | | |
| | | SVKVSCKA | | | | EWMG | | QKFQG | | STVYM | | S | | | | AAGGTCTCCTGCAAGGCTTCTGGAGGC | | SCKASGGTFN | | | |
| | | SG | | | | | | | | ELSSL | | | | | | ACCTTCAACAACCATTTTATCAACTGG | | NHFINWVRRA | | | |
| | | | | | | | | | | RSDDT | | | | | | GTGCGACGGGCCCCTGGACAAGGGCTT | | PGQGLEWMGK | | | |
| | | | | | | | | | | AMYYC | | | | | | GAGTGGATGGGAAAGATCATCCCTATC | | IIPIIDIANS | | | |
| | | | | | | | | | | | | | | | | ATTGATATAGCAAACTCCGCACAGAAG | | AQKFQGRVTI | | | |
| | | | | | | | | | | | | | | | | TTCCAGGGCAGAGTCACGATTACCGCG | | TADKSTSTVY | | | |
| | | | | | | | | | | | | | | | | GACAAATCCACGAGCACAGTCTACATG | | MELSSLRSDD | | | |
| | | | | | | | | | | | | | | | | GAGCTGAGCAGCCTGAGATCTGATGAC | | TAMYYCARRT | | | |
| | | | | | | | | | | | | | | | | ACGGCCATGTATTACTGTGCGCGTCGA | | SSSSEYFDSW | | | |
| | | | | | | | | | | | | | | | | ACTAGCAGTTCGTCTGAGTATTTTGAC | | GQGTLVTVSS | | | |
| | | | | | | | | | | | | | | | | TCCTGGGGCCAGGGAACCCTGGTCACC | | | | | |
| | | | | | | | | | | | | | | | | GTCTCCTCA | | | | | |
| ADI- | IGHV3- | QVQLVQSG | 12 | LIVSSNY | 343 | WVRQA | 437 | TIYPGG | 678 | RFTIS | 927 | VRPIYG | 1178 | WGKGTT | 1291 | CAGGTGCAGCTGGTGCAGTCTGGGGGA | 1475 | QVQLVQSGGG | 1758 | | |
| 75798 | 66 | GGLVQPGG | | MS | | PGKGL | | STFYTD | | RDGSK | | GNPGMD | | VTVSS | | GGCTTGGTCCAGCCTGGGGGGTCCCTG | | LVQPGGSLRL | | | |
| | | SLRLSCAA | | | | EWVS | | SVKG | | NTLYL | | V | | | | AGACTCTCCTGTGCAGCCTCTGGACTC | | SCAASGLIVS | | | |
| | | SG | | | | | | | | EMKSP | | | | | | ATCGTCAGTAGTAACTACATGAGCTGG | | SNYMSWVRQA | | | |
| | | | | | | | | | | RAEDT | | | | | | GTCCGCCAGGCTCCAGGGAAGGGGCTG | | PGKGLEWVST | | | |
| | | | | | | | | | | ALYYC | | | | | | GAGTGGGTCTCAACAATTTATCCCGGT | | IYPGGSTFYT | | | |
| | | | | | | | | | | | | | | | | GGTAGCACATTCTACACAGACTCCGTG | | DSVKGRFTIS | | | |
| | | | | | | | | | | | | | | | | AAGGGCAGATTCACCATCTCCAGAGAC | | RDGSKNTLYL | | | |
| | | | | | | | | | | | | | | | | GGTTCCAAGAACACGCTGTATCTGGAA | | EMKSPRAEDT | | | |
| | | | | | | | | | | | | | | | | ATGAAGAGCCCGAGAGCCGAGGACACG | | ALYYCVRPIY | | | |
| | | | | | | | | | | | | | | | | GCTCTTTACTACTGTGTGAGACCCATA | | GGNPGMDVWG | | | |
| | | | | | | | | | | | | | | | | TACGGTGGTAACCCGGGCATGGACGTC | | KGTTVTVSS | | | |
| | | | | | | | | | | | | | | | | TGGGGCAAAGGGACCACGGTCACCGTC | | | | | |
| | | | | | | | | | | | | | | | | TCCTCA | | | | | |
| ADI- | IGHV3- | QVQLVQSG | 132 | FTFNNYG | 344 | WVRQA | 431 | VIWYDG | 679 | RFTIS | 777 | ARFGYS | 1179 | WGQGTL | 1286 | CAGGTCCAGCTGGTGCAGTCTGGGGGA | 1476 | QVQLVQSGGG | 1759 | | |
| 75782 | 33 | GGVVQPGK | | MH | | PGKGL | | SNKYYA | | RDNSK | | LEATTD | | VTVSS | | GGCGTGGTCCAGCCTGGGAAGTCCCTG | | VVQPGKSLRL | | | |
| | | SLRLSCAT | | | | EWVA | | DSVKG | | NTLYL | | AFHY | | | | AGACTCTCCTGTGCAACGTCTGGATTC | | SCATSGFTFN | | | |
| | | SG | | | | | | | | QMNSL | | | | | | ACCTTCAATAACTATGGCATGCACTGG | | NYGMHWVRQA | | | |
| | | | | | | | | | | RAEDT | | | | | | GTCCGCCAGGCTCCAGGCAAGGGGCTG | | PGKGLEWVAV | | | |
| | | | | | | | | | | AVYYC | | | | | | GAGTGGGTGGCAGTTATATGGTATGAT | | IWYDGSNKYY | | | |
| | | | | | | | | | | | | | | | | GGAAGTAATAAATATTACGCAGACTCC | | ADSVKGRFTI | | | |
| | | | | | | | | | | | | | | | | GTGAAGGGCCGATTCACCATCTCCAGA | | SRDNSKNTLY | | | |
| | | | | | | | | | | | | | | | | GACAATTCCAAGAACACGCTGTATCTG | | LQMNSLRAED | | | |
| | | | | | | | | | | | | | | | | CAAATGAACAGCCTGAGAGCCGAGGAC | | TAVYYCARFG | | | |
| | | | | | | | | | | | | | | | | ACGGCTGTGTATTATTGTGCGAGGTTC | | YSLEATTDAF | | | |
| | | | | | | | | | | | | | | | | GGATATAGTCTAGAGGCCACGACCGAC | | HYWGQGTLVT | | | |
| | | | | | | | | | | | | | | | | GCCTTTCACTACTGGGGCCAGGGAACC | | VSS | | | |
| | | | | | | | | | | | | | | | | CTGGTCACCGTCTCCTCA | | | | | |
| ADI- | IGHV3- | QVQLVQSG | 79 | LIVSSNY | 345 | WVRQA | 489 | VLFAGG | 680 | RFTIS | 777 | ARDLVT | 1180 | WGQGTT | 1287 | CAGGTGCAGCTGGTGCAGTCTGGCGGA | 1477 | QVQLVQSGGG | 1760 | | |
| 75858 | 53 | GGLIQPGG | | MN | | PGNRL | | TTFYAD | | RDNSK | | YGMDV | | VTVSS | | GGCTTGATCCAGCCGGGGGGGTCCCTG | | LIQPGGSLRL | | | |
| | | SLRLSCAA | | | | EWVS | | SVKG | | NTLYL | | | | | | CGACTCTCATGTGCAGCCTCTGGGCTC | | SCAASGLIVS | | | |
| | | SG | | | | | | | | QMNSL | | | | | | ATCGTCAGTAGCAACTACATGAACTGG | | SNYMNWVRQA | | | |
| | | | | | | | | | | RAEDT | | | | | | GTCCGCCAGGCTCCAGGGAACCGGCTG | | PGNRLEWVSV | | | |
| | | | | | | | | | | AVYYC | | | | | | GAGTGGGTCTCAGTTCTTTTTGCCGGT | | LFAGGTTFYA | | | |
| | | | | | | | | | | | | | | | | GGAACCACATTCTACGCAGACTCCGTG | | DSVKGRFTIS | | | |
| | | | | | | | | | | | | | | | | AAGGGCCGATTCACCATCTCCAGAGAC | | RDNSKNTLYL | | | |
| | | | | | | | | | | | | | | | | AATTCCAAGAACACTCTGTATCTCCAA | | QMNSLRAEDT | | | |
| | | | | | | | | | | | | | | | | ATGAACAGCCTGCGCGCCGAGGACACG | | AVYYCARDLV | | | |
| | | | | | | | | | | | | | | | | GCCGTGTATTACTGTGCGAGAGATCTG | | TYGMDVWGQG | | | |
| | | | | | | | | | | | | | | | | GTAACCTACGGTATGGATGTCTGGGGC | | TTVTVSS | | | |
| | | | | | | | | | | | | | | | | CAAGGGACCACGGTCACCGTCTCCTCA | | | | | |
| ADI- | IGHV1- | RYPLVQSG | 133 | GTFSSYT | 346 | WVRQA | 490 | RIFPIL | 681 | RVTIT | 928 | ARETGY | 1181 | WGRGTL | 1288 | AGGTACCCGCTGGTGCAGTCTGGGGCT | 1478 | RYPLVQSGAE | 1761 | | |
| 75712 | 69 | AEVKKPGS | | VI | | PGQGL | | GVTDYA | | ADKST | | SSSGSV | | VTVSS | | GAGGTGAAGAAGCCTGGGTCCTCGGTG | | VKKPGSSVKV | | | |
| | | SVKVSCKA | | | | ECLG | | QKFQD | | GTAYM | | PYFHV | | | | AAGGTCTCCTGCAAGGCTTCTGGAGGC | | SCKASGGTFS | | | |
| | | SG | | | | | | | | ELTSL | | | | | | ACCTTCAGCAGCTATACTGTCATCTGG | | SYTVIWVRQA | | | |
| | | | | | | | | | | RSEDT | | | | | | GTGCGGCAGGCCCCTGGACAAGGGCTT | | PGQGLECLGR | | | |
| | | | | | | | | | | AVYYC | | | | | | GAATGCCTGGGAAGGATCTTCCCTATC | | IFPILGVTDY | | | |
| | | | | | | | | | | | | | | | | CTTGGTGTAACAGACTACGCACAGAAG | | AQKFQDRVTI | | | |
| | | | | | | | | | | | | | | | | TTCCAGGACAGAGTCACGATTACCGCG | | TADKSTGTAY | | | |
| | | | | | | | | | | | | | | | | GACAAATCCACGGGCACAGCCTACATG | | MELTSLRSED | | | |
| | | | | | | | | | | | | | | | | GAACTGACCAGCCTGAGATCTGAGGAC | | TAVYYCARET | | | |
| | | | | | | | | | | | | | | | | ACGGCCGTGTATTACTGTGCGAGAGAG | | GYSSSGSVPY | | | |
| | | | | | | | | | | | | | | | | ACGGGTTACTCTAGTTCGGGGAGTGTT | | FHVWGRGTLV | | | |
| | | | | | | | | | | | | | | | | CCGTACTTCCATGTCTGGGGCCGTGGC | | TVSS | | | |
| | | | | | | | | | | | | | | | | ACCCTGGTCACCGTCTCCTCA | | | | | |
| ADI- | IGHV3- | QVQLQQSG | 134 | ITVSANY | 347 | WVRQA | 437 | VLYSGG | 682 | RFTIS | 777 | ARDLVV | 1182 | WGQGTT | 1287 | CAGGTGCAGCTGCAGCAGTCTGGAGGA | 1479 | QVQLQQSGGG | 1762 | | |
| 75716 | 53 | GGLVQPGG | | MN | | PGKGL | | TTYYAD | | RDNSK | | YGMDV | | VTVSS | | GGCTTGGTCCAGCCTGGGGGGTCCCTG | | LVQPGGSLRL | | | |
| | | SLRLSCVA | | | | EWVS | | SVKG | | NTLYL | | | | | | AGACTCTCCTGTGTAGCCTCTGGGATC | | SCVASGITVS | | | |
| | | SG | | | | | | | | QMNSL | | | | | | ACTGTCAGTGCCAACTACATGAACTGG | | ANYMNWVRQA | | | |
| | | | | | | | | | | RAEDT | | | | | | GTCCGCCAGGCTCCAGGAAAGGGGCTG | | PGKGLEWVSV | | | |
| | | | | | | | | | | AVYYC | | | | | | GAGTGGGTCTCAGTTCTTTATAGCGGT | | LYSGGTTYYA | | | |
| | | | | | | | | | | | | | | | | GGTACCACATACTACGCAGACTCCGTG | | DSVKGRFTIS | | | |
| | | | | | | | | | | | | | | | | AAGGGCCGATTCACCATCTCCAGAGAC | | RDNSKNTLYL | | | |
| | | | | | | | | | | | | | | | | AATTCCAAGAACACCCTATATCTTCAA | | QMNSLRAEDT | | | |
| | | | | | | | | | | | | | | | | ATGAACAGTCTGAGAGCTGAGGACACG | | AVYYCARDLV | | | |
| | | | | | | | | | | | | | | | | GCCGTGTATTACTGTGCGAGAGATCTG | | VYGMDVWGQG | | | |
| | | | | | | | | | | | | | | | | GTCGTCTACGGTATGGACGTCTGGGGC | | TTVTVSS | | | |
| | | | | | | | | | | | | | | | | CAAGGGACCACGGTCACCGTCTCCTCA | | | | | |
| ADI- | IGHV1- | QVQLVQSG | 135 | YTFTTFD | 348 | WVRQA | 491 | WMNPNS | 683 | RVTMT | 929 | ARGGEL | 1183 | WGQGTL | 1286 | CAGGTCCAGCTGGTGCAGTCTGGGGCT | 1480 | QVQLVQSGAE | 1763 | | |
| 75689 | 8 | AEVKKPGA | | IN | | TGQGL | | GYTGYA | | RNTST | | TGLLRH | | VTVSS | | GAGGTGAAGAAGCCTGGGGCCTCAGTT | | VKKPGASVRV | | | |
| | | SVRVSCKA | | | | EWMG | | QKFQG | | STAYV | | | | | | AGGGTCTCCTGCAAGGCTTCTGGATAC | | SCKASGYTFT | | | |
| | | SG | | | | | | | | ELNSL | | | | | | ACCTTCACCACTTTTGATATCAACTGG | | TFDINWVRQA | | | |
| | | | | | | | | | | RSEDT | | | | | | GTGCGACAGGCCACTGGACAAGGGCTT | | TGQGLEWMGW | | | |
| | | | | | | | | | | AVYYC | | | | | | GAGTGGATGGGATGGATGAACCCTAAC | | MNPNSGYTGY | | | |
| | | | | | | | | | | | | | | | | AGTGGTTACACAGGCTATGCACAGAAG | | AQKFQGRVTM | | | |
| | | | | | | | | | | | | | | | | TTCCAGGGCAGAGTCACCATGACCAGG | | TRNTSTSTAY | | | |
| | | | | | | | | | | | | | | | | AACACCTCCACAAGCACAGCCTACGTG | | VELNSLRSED | | | |
| | | | | | | | | | | | | | | | | GAGCTGAACAGCCTGCGATCTGAGGAC | | TAVYYCARGG | | | |
| | | | | | | | | | | | | | | | | ACGGCCGTGTATTACTGTGCGAGAGGG | | ELTGLLRHWG | | | |
| | | | | | | | | | | | | | | | | GGGGAACTTACCGGGCTTTTACGCCAC | | QGTLVTVSS | | | |
| | | | | | | | | | | | | | | | | TGGGGCCAGGGAACCCTGGTCACCGTC | | | | | |
| | | | | | | | | | | | | | | | | TCCTCA | | | | | |
| ADI- | IGHV1- | QVQLVQSG | 35 | YSFTGYY | 349 | WIRQA | 492 | QINPNN | 684 | RVTMT | 930 | SAHGTD | 1184 | WGQGTT | 1287 | CAGGTCCAGCTGGTGCAGTCTGGGGCT | 1481 | QVQLVQSGAE | 1764 | | |
| 75669 | 2 | AEVKKPGA | | MY | | PGQGL | | GGTNYA | | RDTSI | | FDV | | VTVSS | | GAGGTGAAGAAGCCTGGGGCCTCAGTG | | VKKPGASVKV | | | |
| | | SVKVSCKA | | | | EWMG | | QKFQG | | NTAYM | | | | | | AAGGTCTCCTGCAAGGCTTCTGGATAC | | SCKASGYSFT | | | |
| | | SG | | | | | | | | ELRRL | | | | | | AGTTTCACCGGCTACTATATGTACTGG | | GYYMYWIRQA | | | |
| | | | | | | | | | | RSDDT | | | | | | ATACGACAGGCCCCTGGACAAGGGCTT | | PGQGLEWMGQ | | | |
| | | | | | | | | | | AVYYC | | | | | | GAGTGGATGGGACAGATCAACCCTAAC | | INPNNGGTNY | | | |
| | | | | | | | | | | | | | | | | AATGGTGGCACAAACTATGCACAGAAG | | AQKFQGRVTM | | | |
| | | | | | | | | | | | | | | | | TTTCAGGGCAGGGTCACCATGACCAGG | | TRDTSINTAY | | | |
| | | | | | | | | | | | | | | | | GACACGTCCATCAACACAGCCTACATG | | MELRRLRSDD | | | |
| | | | | | | | | | | | | | | | | GAGCTGCGCAGGCTGAGATCTGACGAC | | TAVYYCSAHG | | | |
| | | | | | | | | | | | | | | | | ACGGCCGTGTATTACTGTTCGGCACAT | | TDFDVWGQGT | | | |
| | | | | | | | | | | | | | | | | GGAACTGATTTTGATGTCTGGGGCCAA | | TVTVSS | | | |
| | | | | | | | | | | | | | | | | GGGACCACGGTCACCGTCTCCTCA | | | | | |
| ADI- | IGHV1- | QVQLQQSG | 103 | YTFSSYY | 350 | WVRQA | 433 | IINPSA | 685 | RVTMT | 931 | ARGGIV | 1185 | WGQGTL | 1286 | CAGGTGCAGCTGCAGCAGTCTGGGGCT | 1482 | QVQLQQSGAE | 1765 | | |
| 75839 | 46 | AEVKKPGA | | IH | | PGQGL | | GSTTYA | | SDTST | | PDAKEP | | VTVSS | | GAGGTGAAGAAGCCTGGGGCCTCAGTG | | VKKPGASVKV | | | |
| | | SVKVSCKA | | | | EWMG | | QKFQG | | RTIYI | | FHY | | | | AAGGTTTCCTGCAAGGCATCTGGATAC | | SCKASGYTFS | | | |
| | | SG | | | | | | | | ELSSL | | | | | | ACCTTCAGCAGCTACTATATACATTGG | | SYYIHWVRQA | | | |
| | | | | | | | | | | KSEDT | | | | | | GTGCGACAGGCCCCTGGACAAGGGCTT | | PGQGLEWMGI | | | |
| | | | | | | | | | | AVYYC | | | | | | GAGTGGATGGGAATAATCAACCCTAGC | | INPSAGSTTY | | | |
| | | | | | | | | | | | | | | | | GCTGGTAGCACAACCTACGCACAGAAG | | AQKFQGRVTM | | | |
| | | | | | | | | | | | | | | | | TTCCAGGGCAGAGTCACCATGACCAGC | | TSDTSTRTIY | | | |
| | | | | | | | | | | | | | | | | GACACGTCCACGCGCACAATCTATATT | | IELSSLKSED | | | |
| | | | | | | | | | | | | | | | | GAACTGAGCAGCCTGAAATCTGAGGAC | | TAVYYCARGG | | | |
| | | | | | | | | | | | | | | | | ACGGCCGTCTATTACTGTGCGAGAGGG | | IVPDAKEPFH | | | |
| | | | | | | | | | | | | | | | | GGTATAGTGCCAGATGCTAAGGAACCC | | YWGQGTLVTV | | | |
| | | | | | | | | | | | | | | | | TTTCACTACTGGGGCCAGGGAACCCTG | | SS | | | |
| | | | | | | | | | | | | | | | | GTCACCGTCTCCTCA | | | | | |
| ADI- | IGHV3- | QVQLVQSG | 136 | FTFSNYE | 351 | WVRQG | 493 | YISTSG | 686 | RFTIS | 932 | LTHPQP | 1186 | WGQGTL | 1286 | CAGGTGCAGCTGGTGCAGTCTGGGGGA | 1483 | QVQLVQSGGD | 1766 | | |
| 75754 | 48 | GDLVQPGG | | MN | | PGKGL | | STYYYA | | RDNAR | | QYVRPT | | VTVSS | | GACTTGGTACAGCCTGGAGGGTCCCTG | | LVQPGGSLRL | | | |
| | | SLRLSCVA | | | | EWIS | | DSVKG | | NSLYL | | QVVWFD | | | | AGACTCTCCTGTGTCGCCTCTGGATTC | | SCVASGFTFS | | | |
| | | SG | | | | | | | | QMDSL | | P | | | | ACCTTCAGTAATTATGAGATGAACTGG | | NYEMNWVRQG | | | |
| | | | | | | | | | | RVEDT | | | | | | GTCCGCCAGGGTCCAGGGAAGGGACTG | | PGKGLEWISY | | | |
| | | | | | | | | | | AIYYC | | | | | | GAGTGGATTTCATACATTAGTACTAGT | | ISTSGSTYYY | | | |
| | | | | | | | | | | | | | | | | GGTAGTACCTATTACTACGCAGACTCT | | ADSVKGRFTI | | | |
| | | | | | | | | | | | | | | | | GTGAAGGGCCGATTCACCATATCCAGA | | SRDNARNSLY | | | |
| | | | | | | | | | | | | | | | | GACAACGCCAGGAACTCACTGTATCTG | | LQMDSLRVED | | | |
| | | | | | | | | | | | | | | | | CAAATGGACAGCCTGAGAGTCGAGGAC | | TAIYYCLTHP | | | |
| | | | | | | | | | | | | | | | | ACTGCTATTTATTACTGTCTGACACAC | | QPQYVRPTQV | | | |
| | | | | | | | | | | | | | | | | CCTCAGCCTCAATATGTACGACCCACG | | VWFDPWGQGT | | | |
| | | | | | | | | | | | | | | | | CAGGTGGTCTGGTTCGACCCCTGGGGC | | LVTVSS | | | |
| | | | | | | | | | | | | | | | | CAGGGAACCCTGGTCACCGTCTCCTCA | | | | | |
| ADI- | IGHV1- | QVQLVQSG | 137 | FTFSTSA | 352 | WVRQA | 494 | WIAVGR | 687 | RVKIT | 933 | AAPHCN | 1187 | WGQGTM | 1299 | CAGGTCCAGCTGGTGCAGTCTGGGCCT | 1484 | QVQLVQSGPE | 1767 | | |
| 75743 | 58 | PEVKKPGT | | VQ | | RGQPL | | GITDHA | | RDEST | | RTSCHD | | VTVSS | | GAGGTGAAGAAGCCTGGGACCTCAGTG | | VKKPGTSVKV | | | |
| | | SVKVSCKA | | | | EWIG | | QKFQE | | STAYM | | AFDL | | | | AAGGTCTCCTGCAAGGCTTCTGGATTC | | SCKASGFTFS | | | |
| | | SG | | | | | | | | ELSSL | | | | | | ACCTTTAGTACTTCTGCTGTGCAGTGG | | TSAVQWVRQA | | | |
| | | | | | | | | | | RFDDT | | | | | | GTGCGACAGGCTCGTGGGCAGCCCCTT | | RGQPLEWIGW | | | |
| | | | | | | | | | | AVYYC | | | | | | GAGTGGATAGGATGGATCGCCGTTGGC | | IAVGRGITDH | | | |
| | | | | | | | | | | | | | | | | AGAGGTATCACAGACCACGCACAGAAG | | AQKFQERVKI | | | |
| | | | | | | | | | | | | | | | | TTCCAGGAAAGAGTCAAAATTACCAGG | | TRDESTSTAY | | | |
| | | | | | | | | | | | | | | | | GACGAGTCCACAAGTACAGCCTACATG | | MELSSLRFDD | | | |
| | | | | | | | | | | | | | | | | GAACTGAGCAGCTTGAGATTCGACGAC | | TAVYYCAAPH | | | |
| | | | | | | | | | | | | | | | | ACGGCCGTGTATTACTGTGCGGCACCC | | CNRTSCHDAF | | | |
| | | | | | | | | | | | | | | | | CATTGCAATAGAACCAGCTGCCACGAT | | DLWGQGTMVT | | | |
| | | | | | | | | | | | | | | | | GCTTTTGATCTTTGGGGCCAAGGGACA | | VSS | | | |
| | | | | | | | | | | | | | | | | ATGGTCACCGTCTCTTCA | | | | | |
| ADI- | IGHV3- | QVQLVQCG | 138 | FSFGSYV | 353 | WVRQT | 448 | GISGSG | 688 | RFTLS | 934 | ARGRNG | 1188 | WGQGTL | 1286 | CAGGTCCAGCTGGTGCAGTGTGGGGGA | 1485 | QVQLVQCGGG | 1768 | | |
| 75841 | 23 | GGLVQHGG | | MS | | PGKGL | | GSTYYA | | RDVSK | | WYLGAD | | VTVSS | | GGCTTGGTACAGCATGGGGGGTCCCTA | | LVQHGGSLRL | | | |
| | | SLRLSCAA | | | | EWVS | | EAVRG | | DTLFL | | S | | | | AGACTGTCTTGTGCAGCATCTGGATTC | | SCAASGFSFG | | | |
| | | SG | | | | | | | | QLNSL | | | | | | TCCTTTGGCAGCTATGTCATGAGCTGG | | SYVMSWVRQT | | | |
| | | | | | | | | | | KAADT | | | | | | GTCCGCCAGACTCCAGGGAAGGGGCTG | | PGKGLEWVSG | | | |
| | | | | | | | | | | AIYYC | | | | | | GAGTGGGTCTCAGGTATTAGTGGTAGT | | ISGSGGSTYY | | | |
| | | | | | | | | | | | | | | | | GGCGGTAGCACATACTACGCAGAGGCT | | AEAVRGRFTL | | | |
| | | | | | | | | | | | | | | | | GTGAGGGGCCGGTTCACCTTGTCCAGA | | SRDVSKDTLF | | | |
| | | | | | | | | | | | | | | | | GACGTTTCCAAGGACACACTGTTTTTG | | LQLNSLKAAD | | | |
| | | | | | | | | | | | | | | | | CAACTGAACAGCCTGAAAGCGGCGGAC | | TAIYYCARGR | | | |
| | | | | | | | | | | | | | | | | ACGGCCATATACTACTGTGCGAGAGGG | | NGWYLGADSW | | | |
| | | | | | | | | | | | | | | | | CGCAACGGCTGGTACTTGGGGGGGGAC | | GQGTLVTVSS | | | |
| | | | | | | | | | | | | | | | | TCCTGGGGCCAGGGAACCCTGGTCACC | | | | | |
| | | | | | | | | | | | | | | | | GTCTCCTCA | | | | | |
| ADI- | IGHV3- | RCPLVQSG | 139 | VIVSSNY | 354 | WVRQA | 437 | VIYSGG | 689 | RFTIS | 777 | ARGYGD | 1189 | WGQGTL | 1286 | AGGTGCCCGCTGGTGCAGTCTGGAGGA | 1486 | RCPLVQSGGG | 1769 | | |
| 75879 | 53 | GGLIQPGG | | MN | | PGKGL | | STFYAD | | RDNSK | | YYFDY | | VTVSS | | GGCTTGATCCAGCCTGGGGGGTCCCTG | | LIQPGGSLRL | | | |
| | | SLRLSCAA | | | | EWVS | | SVKG | | NTLYL | | | | | | AGACTCTCCTGTGCAGCCTCTGGAGTC | | SCAASGVIVS | | | |
| | | SG | | | | | | | | QMNSL | | | | | | ATCGTCAGTAGCAACTACATGAACTGG | | SNYMNWVRQA | | | |
| | | | | | | | | | | RAEDT | | | | | | GTCCGCCAGGCTCCAGGGAAGGGGCTG | | PGKGLEWVSV | | | |
| | | | | | | | | | | AVYYC | | | | | | GAGTGGGTCTCAGTTATTTATAGCGGT | | IYSGGSTFYA | | | |
| | | | | | | | | | | | | | | | | GGTAGCACATTCTACGCAGACTCCGTG | | DSVKGRFTIS | | | |
| | | | | | | | | | | | | | | | | AAGGGCCGATTCACCATCTCCAGAGAC | | RDNSKNTLYL | | | |
| | | | | | | | | | | | | | | | | AATTCCAAGAACACGCTGTATCTTCAA | | QMNSLRAEDT | | | |
| | | | | | | | | | | | | | | | | ATGAACAGCCTGAGAGCCGAGGACACG | | AVYYCARGYG | | | |
| | | | | | | | | | | | | | | | | GCCGTGTATTACTGTGCGAGAGGCTAC | | DYYFDYWGQG | | | |
| | | | | | | | | | | | | | | | | GGTGACTACTACTTTGACTACTGGGGC | | TLVTVSS | | | |
| | | | | | | | | | | | | | | | | CAGGGAACCCTGGTCACCGTCTCCTCA | | | | | |
| ADI- | IGHV3- | QVQLVQSG | 140 | FTFSNYG | 355 | WVRQA | 437 | GLSRSG | 690 | RFTIS | 935 | AKDLGL | 1190 | WGQGTL | 1286 | CAGGTGCAGCTGGTGCAGTCTGGGGGA | 1487 | QVQLVQSGGG | 1770 | | |
| 75703 | 23 | GGLVQPGG | | MS | | PGKGL | | DITYYA | | RDNSK | | VVTTEV | | VTVSS | | GGCTTGGTACAGCCTGGGGGGTCCCTG | | LVQPGGSLRL | | | |
| | | SLRLSCEA | | | | EWVS | | DSVKG | | NTLYL | | ADF | | | | AGACTCTCCTGTGAAGCCTCTGGATTC | | SCEASGFTFS | | | |
| | | SG | | | | | | | | QMNSL | | | | | | ACCTTTAGCAACTATGGCATGAGTTGG | | NYGMSWVRQA | | | |
| | | | | | | | | | | RAEDT | | | | | | GTCCGCCAGGCTCCAGGGAAGGGGCTG | | PGKGLEWVSG | | | |
| | | | | | | | | | | AIYYC | | | | | | GAGTGGGTCTCAGGTCTAAGTCGTAGT | | LSRSGDITYY | | | |
| | | | | | | | | | | | | | | | | GGTGATATCACATACTACGCAGACTCC | | ADSVKGRFTI | | | |
| | | | | | | | | | | | | | | | | GTGAAGGGCCGGTTCACCATCTCCAGA | | SRDNSKNTLY | | | |
| | | | | | | | | | | | | | | | | GACAATTCCAAGAACACCCTATATCTG | | LQMNSLRAED | | | |
| | | | | | | | | | | | | | | | | CAAATGAACAGCCTGAGAGCCGAGGAC | | TAIYYCAKDL | | | |
| | | | | | | | | | | | | | | | | ACGGCCATATATTACTGTGCGAAAGAT | | GLVVTTEVAD | | | |
| | | | | | | | | | | | | | | | | CTAGGACTGGTAGTGACTACGGAGGTT | | FWGQGTLVTV | | | |
| | | | | | | | | | | | | | | | | GCTGACTTCTGGGGCCAGGGAACCCTG | | SS | | | |
| | | | | | | | | | | | | | | | | GTCACCGTCTCCTCA | | | | | |
| ADI- | IGHV1- | QVQLVQSG | 50 | GTFSSYS | 356 | WVRQA | 495 | KILPIL | 691 | RVTII | 936 | ARESGY | 1191 | WGRGTL | 1288 | CAGGTCCAGCTGGTGCAGTCTGGGGCT | 1488 | QVQLVQSGAE | 1771 | | |
| 75739 | 69 | AEVKKPGS | | IS | | PGQGP | | GATDYA | | ADKST | | SGSGSV | | VTVSS | | GAGGTGAAGAAGCCTGGGTCGTCGGTG | | VKKPGSSVKV | | | |
| | | SVKVSCKA | | | | EWLG | | QKFQG | | STAYM | | TYFHL | | | | AAGGTCTCCTGCAAGGCCTCTGGAGGC | | SCKASGGTFS | | | |
| | | SG | | | | | | | | ELSSL | | | | | | ACCTTCAGCAGCTATAGTATCTCCTGG | | SYSISWVRQA | | | |
| | | | | | | | | | | RSEDT | | | | | | GTGCGACAGGCCCCTGGACAGGGGCCT | | PGQGPEWLGK | | | |
| | | | | | | | | | | AVYYC | | | | | | GAGTGGTTGGGGAAGATCCTCCCTATC | | ILPILGATDY | | | |
| | | | | | | | | | | | | | | | | CTTGGAGCAACAGACTACGCACAGAAG | | AQKFQGRVTI | | | |
| | | | | | | | | | | | | | | | | TTCCAGGGCAGAGTCACCATTATCGCG | | IADKSTSTAY | | | |
| | | | | | | | | | | | | | | | | GACAAATCCACGAGCACGGCCTATATG | | MELSSLRSED | | | |
| | | | | | | | | | | | | | | | | GAGCTGAGTAGTCTGAGATCTGAGGAC | | TAVYYCARES | | | |
| | | | | | | | | | | | | | | | | ACGGCCGTGTATTATTGTGCGAGAGAG | | GYSGSGSVTY | | | |
| | | | | | | | | | | | | | | | | TCGGGGTACTCTGGTTCGGGGAGTGTT | | FHLWGRGTLV | | | |
| | | | | | | | | | | | | | | | | ACGTACTTCCATCTCTGGGGCCGTGGC | | TVSS | | | |
| | | | | | | | | | | | | | | | | ACCCTGGTCACCGTCTCCTCA | | | | | |
| ADI- | IGHV3- | QVQLVQSG | 141 | FTFSTYG | 215 | WVRQA | 431 | VMWFDG | 692 | RFTIS | 892 | ARESHY | 1192 | WGRGTT | 1294 | CAGGTGCAGCTGGTGCAGTCTGGGGGA | 1489 | QVQLVQSGGG | 1772 | | |
| 75762 | 33 | GGVVQPGK | | MH | | PGKGL | | TNKYYA | | RDNSK | | GEDSEY | | VTVSS | | GGCGTGGTCCAGCCTGGGAAGTCCCTG | | VVQPGKSLRL | | | |
| | | SLRLSCAA | | | | EWVA | | DSVKG | | NTLYL | | YFQYYM | | | | AGACTCTCCTGTGCAGCGTCTGGATTC | | SCAASGFTFS | | | |
| | | SG | | | | | | | | QMNSL | | DV | | | | ACCTTCAGTACCTATGGCATGCACTGG | | TYGMHWVRQA | | | |
| | | | | | | | | | | RAEDT | | | | | | GTCCGCCAGGCTCCAGGCAAGGGGCTG | | PGKGLEWVAV | | | |
| | | | | | | | | | | AVYFC | | | | | | GAGTGGGTGGCAGTTATGTGGTTTGAT | | MWFDGTNKYY | | | |
| | | | | | | | | | | | | | | | | GGAACTAATAAATACTATGCAGACTCC | | ADSVKGRFTI | | | |
| | | | | | | | | | | | | | | | | GTGAAGGGCCGATTCACCATCTCCAGA | | SRDNSKNTLY | | | |
| | | | | | | | | | | | | | | | | GACAATTCCAAGAACACGCTGTATCTG | | LQMNSLRAED | | | |
| | | | | | | | | | | | | | | | | CAAATGAACAGCCTTAGAGCCGAGGAC | | TAVYFCARES | | | |
| | | | | | | | | | | | | | | | | ACGGCTGTGTATTTCTGTGCGAGAGAG | | HYGEDSEYYF | | | |
| | | | | | | | | | | | | | | | | TCCCACTACGGTGAAGATTCCGAATAC | | QYYMDVWGRG | | | |
| | | | | | | | | | | | | | | | | TACTTCCAGTACTACATGGACGTCTGG | | TTVTVSS | | | |
| | | | | | | | | | | | | | | | | GGCAGGGGGACCACGGTCACCGTCTCC | | | | | |
| | | | | | | | | | | | | | | | | TCA | | | | | |
| ADI- | IGHV3- | QVQLVQSG | 12 | FTFSSYE | 307 | WVRQA | 437 | FISNSA | 693 | RFTIS | 937 | ARDLGG | 1193 | WGQGTL | 1286 | CAGGTGCAGCTGGTGCAGTCTGGGGGA | 1490 | QVQLVQSGGG | 1773 | | |
| 75815 | 48 | GGLVQPGG | | MN | | PGKGL | | TARYYA | | RDNAK | | SYSGTF | | VTVSS | | GGCTTGGTACAGCCTGGAGGGTCCCTG | | LVQPGGSLRL | | | |
| | | SLRLSCAA | | | | EWVS | | DSVKG | | NSMYL | | DF | | | | AGACTCTCCTGTGCAGCCTCTGGATTC | | SCAASGFTFS | | | |
| | | SG | | | | | | | | QMNSL | | | | | | ACCTTCAGTAGTTATGAAATGAACTGG | | SYEMNWVRQA | | | |
| | | | | | | | | | | RAEDT | | | | | | GTCCGCCAGGCTCCAGGGAAGGGGCTG | | PGKGLEWVSF | | | |
| | | | | | | | | | | AIYYC | | | | | | GAGTGGGTTTCATTCATTAGCAATAGT | | ISNSATARYY | | | |
| | | | | | | | | | | | | | | | | GCTACTGCCAGATACTACGCAGACTCT | | ADSVKGRFTI | | | |
| | | | | | | | | | | | | | | | | GTGAAGGGCCGATTCACCATCTCCAGA | | SRDNAKNSMY | | | |
| | | | | | | | | | | | | | | | | GACAACGCCAAGAACTCCATGTATCTG | | LQMNSLRAED | | | |
| | | | | | | | | | | | | | | | | CAAATGAACAGCCTGAGAGCCGAGGAC | | TAIYYCARDL | | | |
| | | | | | | | | | | | | | | | | ACGGCTATTTATTACTGTGCGAGAGAT | | GGSYSGTFDF | | | |
| | | | | | | | | | | | | | | | | TTGGGAGGGAGCTACTCGGGGACCTTT | | WGQGTLVTVS | | | |
| | | | | | | | | | | | | | | | | GACTTCTGGGGCCAGGGAACCCTGGTC | | S | | | |
| | | | | | | | | | | | | | | | | ACCGTCTCCTCA | | | | | |
| ADI- | IGHV3- | EVQLVQSG | 142 | FTFSSYS | 357 | WVRQA | 431 | VISYDG | 694 | RFTIS | 938 | ARGPHF | 1194 | WGQGTL | 1286 | GAGGTGCAGCTGGTGCAGTCTGGGGGA | 1491 | EVQLVQSGGG | 1774 | | |
| 75765 | 30 | GGVVQPGG | | VH | | PGKGL | | NNKYYA | | RDNSK | | DWVPHT | | VTVSS | | GGCGTGGTCCAGCCTGGGGGGTCCCTG | | VVQPGGSLRL | | | |
| | | SLRLSCAA | | | | EWVA | | ASVKG | | DTLYL | | FDY | | | | AGACTCTCCTGTGCAGCCTCTGGATTC | | SCAASGFTFS | | | |
| | | SG | | | | | | | | EMNSL | | | | | | ACCTTCAGTAGCTATTCTGTACACTGG | | SYSVHWVRQA | | | |
| | | | | | | | | | | RDEDT | | | | | | GTCCGCCAGGCTCCAGGCAAGGGGCTA | | PGKGLEWVAV | | | |
| | | | | | | | | | | AVYYC | | | | | | GAGTGGGTGGCAGTTATATCATATGAT | | ISYDGNNKYY | | | |
| | | | | | | | | | | | | | | | | GGAAATAACAAGTACTACGCAGCCTCC | | AASVKGRFTI | | | |
| | | | | | | | | | | | | | | | | GTGAAGGGCCGATTCACCATCTCCAGA | | SRDNSKDTLY | | | |
| | | | | | | | | | | | | | | | | GACAATTCCAAGGACACGCTGTATCTT | | LEMNSLRDED | | | |
| | | | | | | | | | | | | | | | | GAAATGAACAGCCTGAGAGATGAGGAC | | TAVYYCARGP | | | |
| | | | | | | | | | | | | | | | | ACGGCTGTATATTACTGTGCGAGAGGT | | HFDWVPHTFD | | | |
| | | | | | | | | | | | | | | | | CCACATTTTGACTGGGTCCCGCATACC | | YWGQGTLVTV | | | |
| | | | | | | | | | | | | | | | | TTTGACTACTGGGGCCAGGGAACCCTG | | SS | | | |
| | | | | | | | | | | | | | | | | GTCACCGTCTCCTCA | | | | | |
| ADI- | IGHV5- | QVQLVQSG | 143 | YYFTTYW | 358 | WVRQM | 496 | RIDPSD | 695 | HVTMS | 939 | ARECGG | 1195 | WGQGTT | 1287 | CAGGTGCAGCTGGTGCAGTCTGGAACA | 1492 | QVQLVQSGTE | 1775 | | |
| 75705 | 10-1 | TEVKKPGE | | IT | | PGKGL | | SYTTYS | | IDKSI | | DCYFPE | | VTVSS | | GAAGTGAAAAAGCCCGGGGAGTCTCTG | | VKKPGESLTI | | | |
| | | SLTISCKT | | | | EWLG | | PSFQG | | TTAYL | | PLSHTA | | | | ACGATCTCCTGTAAGACTTCTGGGTAC | | SCKTSGYYFT | | | |
| | | SG | | | | | | | | QWTSL | | FDI | | | | TACTTTACCACCTACTGGATCACCTGG | | TYWITWVRQM | | | |
| | | | | | | | | | | KASDT | | | | | | GTGCGCCAGATGCCCGGGAAAGGCCTG | | PGKGLEWLGR | | | |
| | | | | | | | | | | AMYYC | | | | | | GAGTGGCTGGGGAGGATTGATCCTAGT | | IDPSDSYTTY | | | |
| | | | | | | | | | | | | | | | | GACTCCTATACCACCTACAGCCCGTCC | | SPSFQGHVTM | | | |
| | | | | | | | | | | | | | | | | TTCCAAGGCCACGTCACCATGTCAATT | | SIDKSITTAY | | | |
| | | | | | | | | | | | | | | | | GACAAGTCCATCACCACTGCCTACCTG | | LQWTSLKASD | | | |
| | | | | | | | | | | | | | | | | CAGTGGACCAGCCTGAAGGCCTCGGAC | | TAMYYCAREC | | | |
| | | | | | | | | | | | | | | | | ACCGCCATGTATTACTGTGCGAGAGAA | | GGDCYFPEPL | | | |
| | | | | | | | | | | | | | | | | TGTGGTGGTGACTGCTATTTTCCGGAG | | SHTAFDIWGQ | | | |
| | | | | | | | | | | | | | | | | CCCCTCTCCCATACTGCTTTTGATATC | | GTTVTVSS | | | |
| | | | | | | | | | | | | | | | | TGGGGCCAAGGGACCACGGTCACCGTC | | | | | |
| | | | | | | | | | | | | | | | | TCCTCA | | | | | |
| ADI- | IGHV3- | QVQLVQSG | 144 | FTLRNYA | 359 | WVRQA | 497 | FVAYGR | 696 | RFTIS | 940 | AREPHC | 1196 | WGQGTT | 1287 | CAGGTGCAGCTGGTGCAGTCTGGGGGA | 1493 | QVQLVQSGGG | 1776 | | |
| 75678 | 30 | GGVVQPGG | | MH | | PGKGL | | SSEDYA | | RDNAK | | TGGVCD | | VTVSS | | GGCGTGGTCCAGCCTGGGGGGTCCCTG | | VVQPGGSLRL | | | |
| | | SLRLSCEG | | | | ECVA | | DSVKG | | NTVYL | | AFDV | | | | AGACTCTCCTGTGAAGGCTCTGGATTC | | SCEGSGFTLR | | | |
| | | SG | | | | | | | | EMNSL | | | | | | ACCCTCAGAAACTACGCTATGCACTGG | | NYAMHWVRQA | | | |
| | | | | | | | | | | RVEDA | | | | | | GTCCGCCAGGCTCCAGGCAAGGGGCTG | | PGKGLECVAF | | | |
| | | | | | | | | | | AIYYC | | | | | | GAGTGCGTGGCTTTTGTTGCATATGGC | | VAYGRSSEDY | | | |
| | | | | | | | | | | | | | | | | AGAAGTAGTGAGGACTACGCAGACTCC | | ADSVKGRFTI | | | |
| | | | | | | | | | | | | | | | | GTGAAGGGCCGATTCACCATCTCCAGA | | SRDNAKNTVY | | | |
| | | | | | | | | | | | | | | | | GACAATGCCAAGAACACGGTCTATCTA | | LEMNSLRVED | | | |
| | | | | | | | | | | | | | | | | GAAATGAACAGTCTGAGAGTTGAGGAC | | AAIYYCAREP | | | |
| | | | | | | | | | | | | | | | | GCGGCGATCTATTACTGTGCGAGAGAG | | HCTGGVCDAF | | | |
| | | | | | | | | | | | | | | | | CCCCATTGTACTGGAGGTGTTTGTGAT | | DVWGQGTTVT | | | |
| | | | | | | | | | | | | | | | | GCTTTTGATGTCTGGGGCCAAGGGACC | | VSS | | | |
| | | | | | | | | | | | | | | | | ACGGTCACCGTCTCCTCA | | | | | |
| ADI- | IGHV5- | QVQLVQSG | 145 | YRFTTYW | 360 | WVRQM | 476 | KIDPSD | 697 | HVSIS | 941 | ARLSAD | 1197 | WGQGTT | 1287 | CAGGTGCAGCTGGTGCAGTCTGGAGCA | 1494 | QVQLVQSGAE | 1777 | | |
| 75707 | 10-1 | AEVKKPGE | | IS | | PGKGL | | SYTNYN | | ADKSI | | DYIWGN | | VTVSS | | GAGGTGAAAAAGCCCGGGGAGTCTCTG | | VKKPGESLRI | | | |
| | | SLRISCKG | | | | EWMG | | PSFQG | | STAYL | | YGGGDA | | | | AGGATCTCCTGTAAGGGTTCTGGATAC | | SCKGSGYRFT | | | |
| | | SG | | | | | | | | QWSSL | | FDI | | | | AGATTTACCACCTACTGGATCAGCTGG | | TYWISWVRQM | | | |
| | | | | | | | | | | RASDT | | | | | | GTGCGCCAGATGCCCGGAAAAGGCCTG | | PGKGLEWMGK | | | |
| | | | | | | | | | | AMYYC | | | | | | GAGTGGATGGGGAAGATTGATCCTAGT | | IDPSDSYTNY | | | |
| | | | | | | | | | | | | | | | | GACTCTTATACCAACTACAACCCGTCC | | NPSFQGHVSI | | | |
| | | | | | | | | | | | | | | | | TTCCAAGGCCACGTCAGCATCTCAGCT | | SADKSISTAY | | | |
| | | | | | | | | | | | | | | | | GACAAGTCCATCAGCACTGCCTACCTG | | LQWSSLRASD | | | |
| | | | | | | | | | | | | | | | | CAGTGGAGCAGCCTGAGGGCCTCGGAC | | TAMYYCARLS | | | |
| | | | | | | | | | | | | | | | | ACCGCCATGTATTACTGTGCGAGACTT | | ADDYIWGNYG | | | |
| | | | | | | | | | | | | | | | | AGCGCAGATGATTACATTTGGGGGAAT | | GGDAFDIWGQ | | | |
| | | | | | | | | | | | | | | | | TATGGCGGAGGGGATGCATTTGATATC | | GTTVTVSS | | | |
| | | | | | | | | | | | | | | | | TGGGGCCAAGGGACAACGGTCACCGTC | | | | | |
| | | | | | | | | | | | | | | | | TCCTCA | | | | | |
| ADI- | IGHV3- | QVQLVQSG | 146 | FNFGDYS | 361 | WVRQA | 437 | FIRSKT | 698 | RFTIS | 942 | CREIEG | 1198 | WGKGTT | 1291 | CAGGTCCAGCTGGTGCAGTCTGGGGGA | 1495 | QVQLVQSGGG | 1778 | | |
| 75796 | 49 | GGLVQPGR | | MS | | PGKGL | | YGGTTH | | RDDSK | | TYNYGS | | VTVSS | | GGCTTGGTACAGCCAGGGCGGTCCCTG | | LVQPGRSLRL | | | |
| | | SLRLSCTT | | | | EWVS | | YAASVK | | RIAYL | | NYFYYI | | | | AGACTCTCCTGTACAACTTCTGGATTC | | SCTTSGFNFG | | | |
| | | SG | | | | | | IG | | QMNSL | | DV | | | | AACTTTGGTGATTATTCTATGAGCTGG | | DYSMSWVRQA | | | |
| | | | | | | | | | | KTEDT | | | | | | GTCCGCCAGGCTCCAGGGAAGGGGCTG | | PGKGLEWVSF | | | |
| | | | | | | | | | | AEYYC | | | | | | GAGTGGGTAAGTTTCATTAGAAGCAAG | | IRSKTYGGTT | | | |
| | | | | | | | | | | | | | | | | ACTTATGGAGGGACAACACATTACGCC | | HYAASVKGRF | | | |
| | | | | | | | | | | | | | | | | GCGTCTGTGAAAGGCAGATTCACCATC | | TISRDDSKRI | | | |
| | | | | | | | | | | | | | | | | TCAAGAGATGATTCCAAACGCATCGCC | | AYLQMNSLKT | | | |
| | | | | | | | | | | | | | | | | TATCTGCAAATGAACAGCCTGAAGACC | | EDTAEYYCCR | | | |
| | | | | | | | | | | | | | | | | GAGGACACAGCCGAGTATTATTGTTGT | | EIEGTYNYGS | | | |
| | | | | | | | | | | | | | | | | AGAGAGATTGAAGGGACATATAACTAT | | NYFYYIDVWG | | | |
| | | | | | | | | | | | | | | | | GGTTCGAACTACTTCTACTACATCGAC | | KGTTVTVSS | | | |
| | | | | | | | | | | | | | | | | GTCTGGGGCAAAGGGACCACGGTCACC | | | | | |
| | | | | | | | | | | | | | | | | GTCTCCTCA | | | | | |
| ADI- | IGHV3- | QVQLVQSG | 12 | FTFSSYS | 362 | WVRQA | 437 | YISSRS | 699 | RFTIS | 854 | ARDDRR | 1199 | WGIGTT | 1300 | CAGGTGCAGCTGGTGCAGTCTGGGGGA | 1496 | QVQLVQSGGG | 1779 | | |
| 75673 | 48 | GGLVQPGG | | MN | | PGKGL | | STISYA | | RDNAK | | LGDYYY | | VTVSS | | GGCTTGGTACAGCCTGGGGGGTCCCTG | | LVQPGGSLRL | | | |
| | | SLRLSCAA | | | | EWVS | | DSVKG | | NSLYL | | YYMDV | | | | AGACTCTCCTGTGCAGCCTCTGGATTC | | SCAASGFTFS | | | |
| | | SG | | | | | | | | QMNSL | | | | | | ACCTTCAGTAGTTATAGCATGAACTGG | | SYSMNWVRQA | | | |
| | | | | | | | | | | RAEDT | | | | | | GTCCGCCAGGCTCCAGGGAAGGGGCTG | | PGKGLEWVSY | | | |
| | | | | | | | | | | AVYYC | | | | | | GAATGGGTTTCATACATTAGTAGTAGG | | ISSRSSTISY | | | |
| | | | | | | | | | | | | | | | | AGTAGCACCATATCCTATGCAGACTCT | | ADSVKGRFTI | | | |
| | | | | | | | | | | | | | | | | GTGAAGGGCCGCTTCACCATCTCCAGA | | SRDNAKNSLY | | | |
| | | | | | | | | | | | | | | | | GACAATGCCAAGAACTCACTGTATCTG | | LQMNSLRAED | | | |
| | | | | | | | | | | | | | | | | CAAATGAACAGCCTGAGAGCCGAGGAC | | TAVYYCARDD | | | |
| | | | | | | | | | | | | | | | | ACGGCTGTGTATTACTGTGCGAGAGAT | | RRLGDYYYYY | | | |
| | | | | | | | | | | | | | | | | GATCGGAGACTAGGAGACTACTACTAC | | MDVWGIGTTV | | | |
| | | | | | | | | | | | | | | | | TATTACATGGACGTCTGGGGCATAGGG | | TVSS | | | |
| | | | | | | | | | | | | | | | | ACCACGGTCACCGTCTCCTCA | | | | | |
| ADI- | IGHV3- | QVQLVQSG | 79 | FIVSRNY | 302 | WVRQA | 437 | LIYAGG | 700 | RFTIS | 777 | ARDRGP | 1200 | WGQGTL | 1286 | CAGGTGCAGCTGGTGCAGTCTGGAGGA | 1497 | QVQLVQSGGG | 1780 | | |
| 75566 | 53 | GGLIQPGG | | MS | | PGKGL | | TTYYAD | | RDNSK | | LLHDY | | VTVSS | | GGCTTGATCCAGCCTGGGGGGTCCCTG | | LIQPGGSLRL | | | |
| | | SLRLSCAA | | | | EWVS | | SVKG | | NTLYL | | | | | | AGACTCTCCTGTGCAGCCTCTGGATTC | | SCAASGFIVS | | | |
| | | SG | | | | | | | | QMNSL | | | | | | ATCGTCAGTAGAAACTACATGAGCTGG | | RNYMSWVRQA | | | |
| | | | | | | | | | | RAEDT | | | | | | GTCCGCCAGGCTCCAGGGAAGGGGCTG | | PGKGLEWVSL | | | |
| | | | | | | | | | | AVYYC | | | | | | GAGTGGGTCTCACTTATTTATGCCGGC | | IYAGGTTYYA | | | |
| | | | | | | | | | | | | | | | | GGTACCACATACTACGCAGACTCCGTG | | DSVKGRFTIS | | | |
| | | | | | | | | | | | | | | | | AAGGGCCGATTCACCATCTCCAGAGAC | | RDNSKNTLYL | | | |
| | | | | | | | | | | | | | | | | AATTCCAAGAACACGCTGTATCTTCAA | | QMNSLRAEDT | | | |
| | | | | | | | | | | | | | | | | ATGAACAGCCTGAGAGCCGAGGACACG | | AVYYCARDRG | | | |
| | | | | | | | | | | | | | | | | GCCGTTTATTACTGTGCAAGAGATAGG | | PLLHDYWGQG | | | |
| | | | | | | | | | | | | | | | | GGGCCTTTACTACACGACTACTGGGGC | | TLVTVSS | | | |
| | | | | | | | | | | | | | | | | CAGGGCACCCTGGTCACCGTCTCCTCA | | | | | |
| ADI- | IGHV1- | QVQLLQSG | 147 | GTFSSYT | 363 | WMRQA | 498 | KIFPIL | 701 | RITII | 943 | ARESGY | 1191 | WGRGTL | 1288 | CAGGTGCAGCTGCTGCAGTCTGGGGCT | 1498 | QVQLLQSGAE | 1781 | | |
| 75706 | 69 | AEVKKPGS | | IS | | PGQGP | | GATDYA | | ADKST | | SGSGSV | | VTVSS | | GAGGTGAAGAAGCCTGGGTCCTCGGTG | | VKKPGSSVKV | | | |
| | | SVKVSCKA | | | | EWLG | | QKFQG | | NTAYL | | TYFHL | | | | AAGGTCTCCTGCAAGGCTTCTGGAGGC | | SCKASGGTFS | | | |
| | | SG | | | | | | | | ELSSL | | | | | | ACCTTCAGCAGCTATACTATCTCCTGG | | SYTISWMRQA | | | |
| | | | | | | | | | | RSEDT | | | | | | ATGCGACAGGCCCCTGGACAGGGGCCT | | PGQGPEWLGK | | | |
| | | | | | | | | | | AVYYC | | | | | | GAGTGGTTGGGAAAGATCTTCCCTATC | | IFPILGATDY | | | |
| | | | | | | | | | | | | | | | | CTTGGAGCAACAGACTACGCACAGAAG | | AQKFQGRITI | | | |
| | | | | | | | | | | | | | | | | TTCCAGGGCAGAATCACCATTATCGCG | | IADKSTNTAY | | | |
| | | | | | | | | | | | | | | | | GACAAGTCCACGAACACGGCCTATTTG | | LELSSLRSED | | | |
| | | | | | | | | | | | | | | | | GAGCTGAGCAGCCTGAGATCTGAGGAC | | TAVYYCARES | | | |
| | | | | | | | | | | | | | | | | ACGGCCGTGTATTATTGTGCGAGAGAG | | GYSGSGSVTY | | | |
| | | | | | | | | | | | | | | | | TCGGGGTACTCTGGTTCGGGGAGTGTT | | FHLWGRGTLV | | | |
| | | | | | | | | | | | | | | | | ACGTACTTCCATCTCTGGGGCCGTGGC | | TVSS | | | |
| | | | | | | | | | | | | | | | | ACCCTGGTCACCGTCTCCTCA | | | | | |
| ADI- | IGHV1- | QVQLVQSG | 50 | GTF- | 364 | WVRQA | 433 | RIIPIL | 702 | RVTIT | 944 | AREGGT | 1201 | WGQGTL | 1286 | CAGGTGCAGCTGGTGCAGTCTGGGGCT | 1499 | QVQLVQSGAE | 1782 | | |
| 75699 | 69 | AEVKKPGS | | SYNIN | | PGQGL | | GIPHYA | | ADKST | | TTAYFP | | VTVSS | | GAGGTGAAGAAGCCTGGGTCCTCGGTG | | VKKPGSSVKV | | | |
| | | SVKVSCKA | | | | EWMG | | QRFQG | | TTLFM | | H | | | | AAGGTCTCCTGCAAGGCTTCTGGAGGC | | SCKASGGTFS | | | |
| | | SG | | | | | | | | ELSSL | | | | | | ACTTTCAGTTATAATATAAACTGGGTG | | YNINWVRQAP | | | |
| | | | | | | | | | | RSEDT | | | | | | CGACAGGCCCCTGGACAAGGGCTTGAG | | GQGLEWMGRI | | | |
| | | | | | | | | | | AIYYC | | | | | | TGGATGGGAAGGATCATCCCTATCCTT | | IPILGIPHYA | | | |
| | | | | | | | | | | | | | | | | GGTATACCACACTACGCACAGAGGTTC | | QRFQGRVTIT | | | |
| | | | | | | | | | | | | | | | | CAGGGCAGAGTCACGATTACCGCGGAC | | ADKSTTTLFM | | | |
| | | | | | | | | | | | | | | | | AAATCCACGACTACCCTGTTCATGGAG | | ELSSLRSEDT | | | |
| | | | | | | | | | | | | | | | | CTGAGCAGCCTGAGATCTGAGGACACG | | AIYYCAREGG | | | |
| | | | | | | | | | | | | | | | | GCCATATATTACTGTGCGAGAGAGGGT | | TTTAYFPHWG | | | |
| | | | | | | | | | | | | | | | | GGTACCACCACTGCATACTTCCCGCAC | | QGTLVTVSS | | | |
| | | | | | | | | | | | | | | | | TGGGGCCAGGGCACCCTGGTCACCGTC | | | | | |
| | | | | | | | | | | | | | | | | TCCTCA | | | | | |
| ADI- | IGHV1- | QVQLQQSG | 103 | HTFTGYF | 365 | WVRQA | 433 | RINPNS | 703 | RVTMT | 945 | AREPGR | 1202 | WGQGTL | 1286 | CAGGTGCAGCTGCAGCAGTCTGGGGCT | 1500 | QVQLQQSGAE | 1783 | | |
| 75591 | 2 | AEVKKPGA | | IY | | PGQGL | | GDTDYA | | RDTSI | | DILTGD | | VTVSS | | GAGGTGAAGAAGCCTGGGGCCTCGGTG | | VKKPGASVKV | | | |
| | | SVKVSCKA | | | | EWMG | | QKFQG | | STAYM | | YWDS | | | | AAGGTCTCCTGCAAGGCTTCTGGACAC | | SCKASGHTFT | | | |
| | | SG | | | | | | | | ELSRL | | | | | | ACCTTCACCGGCTACTTTATCTACTGG | | GYFIYWVRQA | | | |
| | | | | | | | | | | TSDDT | | | | | | GTGCGACAGGCCCCTGGACAAGGGCTT | | PGQGLEWMGR | | | |
| | | | | | | | | | | AMYYC | | | | | | GAGTGGATGGGACGGATCAATCCTAAT | | INPNSGDTDY | | | |
| | | | | | | | | | | | | | | | | AGTGGTGACACAGACTATGCACAGAAG | | AQKFQGRVTM | | | |
| | | | | | | | | | | | | | | | | TTCCAGGGCAGGGTCACCATGACCAGG | | TRDTSISTAY | | | |
| | | | | | | | | | | | | | | | | GACACGTCCATCAGCACAGCCTACATG | | MELSRLTSDD | | | |
| | | | | | | | | | | | | | | | | GAGCTGAGCAGGCTGACCTCTGACGAC | | TAMYYCAREP | | | |
| | | | | | | | | | | | | | | | | ACGGCCATGTATTACTGTGCGAGAGAG | | GRDILTGDYW | | | |
| | | | | | | | | | | | | | | | | CCGGGGAGGGATATTTTGACTGGTGAT | | DSWGQGTLVT | | | |
| | | | | | | | | | | | | | | | | TATTGGGACTCGTGGGGCCAGGGAACC | | VSS | | | |
| | | | | | | | | | | | | | | | | CTGGTCACCGTCTCCTCA | | | | | |
| ADI- | IGHV4- | QVQLQQSG | 44 | GSIGSSH | 366 | WVRQT | 499 | EIYQSV | 704 | RVTMS | 946 | ARRGSG | 1203 | WGQGTT | 1287 | CAGGTGCAGCTGCAGCAGTCGGGCCCA | 1501 | QVQLQQSGPG | 1784 | | |
| 75698 | 4 | PGLVKPSG | | WWS | | PGKGL | | STNYNP | | VDKSK | | YCSGGT | | VTVSS | | GGACTGGTGAAGCCTTCGGGGACCCTG | | LVKPSGTLSL | | | |
| | | TLSLTCAV | | | | EWIG | | SLES | | NQFSL | | CYQIPA | | | | TCCCTCACCTGCGCTGTCTCTGGTGGC | | TCAVSGGSIG | | | |
| | | SG | | | | | | | | KLTSV | | DAFAI | | | | TCCATCGGGAGTAGTCACTGGTGGAGT | | SSHWWSWVRQ | | | |
| | | | | | | | | | | TAADT | | | | | | TGGGTCCGCCAGACCCCAGGGAAGGGG | | TPGKGLEWIG | | | |
| | | | | | | | | | | ALYYC | | | | | | CTGGAGTGGATTGGGGAAATCTATCAA | | EIYQSVSTNY | | | |
| | | | | | | | | | | | | | | | | AGTGTGAGCACCAACTACAACCCGTCC | | NPSLESRVTM | | | |
| | | | | | | | | | | | | | | | | CTCGAGAGTCGGGTCACCATGTCAGTA | | SVDKSKNQFS | | | |
| | | | | | | | | | | | | | | | | GACAAGTCCAAGAACCAGTTCTCCCTG | | LKLTSVTAAD | | | |
| | | | | | | | | | | | | | | | | AAGCTGACCTCTGTGACCGCCGCGGAC | | TALYYCARRG | | | |
| | | | | | | | | | | | | | | | | ACGGCCCTCTATTACTGTGCGAGAAGG | | SGYCSGGTCY | | | |
| | | | | | | | | | | | | | | | | GGGAGTGGATATTGTAGTGGTGGCACC | | QIPADAFAIW | | | |
| | | | | | | | | | | | | | | | | TGCTACCAAATCCCCGCCGATGCTTTT | | GQGTTVTVSS | | | |
| | | | | | | | | | | | | | | | | GCTATCTGGGGCCAAGGGACCACGGTC | | | | | |
| | | | | | | | | | | | | | | | | ACCGTCTCCTCA | | | | | |
| ADI- | IGHV1- | QVQLVQSG | 35 | YTFTSYG | 367 | WVRQA | 433 | WISTYN | 705 | RVTMT | 947 | ARVFSF | 1204 | WGQGTL | 1286 | CAGGTCCAGCTGGTGCAGTCTGGAGCT | 1502 | QVQLVQSGAE | 1785 | | |
| 75878 | 18 | AEVKKPGA | | IS | | PGQGL | | GNTNYA | | TDTST | | YDSRAF | | VTVSS | | GAGGTGAAGAAGCCTGGGGCCTCAGTG | | VKKPGASVKV | | | |
| | | SVKVSCKA | | | | EWMG | | QKLQG | | GTAYM | | TIGGTA | | | | AAGGTCTCCTGCAAGGCTTCTGGTTAC | | SCKASGYTFT | | | |
| | | SG | | | | | | | | ELRSL | | YYFDY | | | | ACCTTTACCAGCTACGGTATCAGCTGG | | SYGISWVRQA | | | |
| | | | | | | | | | | RSDDT | | | | | | GTGCGACAGGCCCCTGGACAGGGGCTT | | PGQGLEWMGW | | | |
| | | | | | | | | | | AVYYC | | | | | | GAGTGGATGGGATGGATCAGCACTTAC | | ISTYNGNTNY | | | |
| | | | | | | | | | | | | | | | | AATGGTAACACAAACTATGCACAGAAG | | AQKLQGRVTM | | | |
| | | | | | | | | | | | | | | | | CTCCAGGGCAGAGTCACCATGACCACA | | TTDTSTGTAY | | | |
| | | | | | | | | | | | | | | | | GACACATCCACGGGCACAGCCTACATG | | MELRSLRSDD | | | |
| | | | | | | | | | | | | | | | | GAGTTGAGGAGCCTGAGATCTGACGAC | | TAVYYCARVF | | | |
| | | | | | | | | | | | | | | | | ACGGCCGTATATTACTGTGCGAGAGTT | | SFYDSRAFTI | | | |
| | | | | | | | | | | | | | | | | TTCAGTTTCTATGATAGTCGTGCTTTT | | GGTAYYFDYW | | | |
| | | | | | | | | | | | | | | | | ACCATCGGGGGGACTGCGTACTACTTT | | GQGTLVTVSS | | | |
| | | | | | | | | | | | | | | | | GACTACTGGGGCCAGGGAACCCTGGTC | | | | | |
| | | | | | | | | | | | | | | | | ACCGTCTCCTCA | | | | | |
| ADI- | IGHV3- | QVQLVQSG | 148 | FTFRNFG | 368 | WVRQA | 431 | TTFYDG | 706 | RFTVS | 948 | AKDGGY | 1205 | WGQGTL | 1286 | CAGGTCCAGCTGGTGCAGTCTGGGGGA | 1503 | QVQLVQSGGG | 1786 | | |
| 75718 | 30 | GGVVQPGM | | MH | | PGKGL | | SDKFYA | | RDNFD | | GDYEIE | | VTVSS | | GGCGTTGTCCAGCCTGGGATGTCCCTG | | VVQPGMSLRL | | | |
| | | SLRLSCAA | | | | EWVA | | DSVKG | | NILYL | | S | | | | AGACTCTCCTGTGCAGCCTCTAGATTC | | SCAASRFTFR | | | |
| | | SR | | | | | | | | QMNNL | | | | | | ACTTTTCGTAACTTTGGCATGCACTGG | | NFGMHWVRQA | | | |
| | | | | | | | | | | RPEDT | | | | | | GTCCGCCAGGCTCCCGGCAAGGGACTG | | PGKGLEWVAT | | | |
| | | | | | | | | | | AMYYC | | | | | | GAGTGGGTGGCAACTACATTTTATGAC | | TFYDGSDKFY | | | |
| | | | | | | | | | | | | | | | | GGCAGTGATAAATTCTATGCAGACTCC | | ADSVKGRFTV | | | |
| | | | | | | | | | | | | | | | | GTGAAGGGCCGATTCACCGTCTCCAGA | | SRDNFDNILY | | | |
| | | | | | | | | | | | | | | | | GACAATTTCGACAATATCCTGTATCTG | | LQMNNLRPED | | | |
| | | | | | | | | | | | | | | | | CAAATGAACAACCTGAGACCTGAGGAC | | TAMYYCAKDG | | | |
| | | | | | | | | | | | | | | | | ACGGCTATGTATTACTGTGCGAAAGAT | | GYGDYEIESW | | | |
| | | | | | | | | | | | | | | | | GGGGGCTACGGTGACTACGAAATTGAG | | GQGTLVTVSS | | | |
| | | | | | | | | | | | | | | | | TCCTGGGGCCAGGGAACCCTGGTCACC | | | | | |
| | | | | | | | | | | | | | | | | GTCTCCTCA | | | | | |
| ADI- | IGHV3- | QVQLVQSG | 149 | FTVSSNY | 369 | WVRQT | 448 | VIYPGG | 649 | RFTIS | 949 | ARFHDI | 1206 | WGQGTT | 1287 | CAGGTGCAGCTGGTGCAGTCTGGAGGA | 1504 | QVQLVQSGGG | 1787 | | |
| 75836 | 53 | GGLIQPGG | | MN | | PGKGL | | STFYAD | | RDNSK | | STRYSD | | VTVSS | | GGCTTGATCCAGCCTGGGGGGTCCCTG | | LIQPGGSLRL | | | |
| | | SLRLSCRA | | | | EWVS | | SVKG | | NTLYV | | AFDI | | | | AGACTCTCCTGTAGAGCCTCTGGGTTT | | SCRASGFTVS | | | |
| | | SG | | | | | | | | QLNSL | | | | | | ACCGTCAGTAGCAACTACATGAACTGG | | SNYMNWVRQT | | | |
| | | | | | | | | | | RAEDT | | | | | | GTCCGCCAGACTCCAGGGAAGGGGCTA | | PGKGLEWVSV | | | |
| | | | | | | | | | | AVYYC | | | | | | GAGTGGGTCTCAGTTATTTATCCCGGA | | IYPGGSTFYA | | | |
| | | | | | | | | | | | | | | | | GGTAGCACATTCTACGCAGACTCCGTG | | DSVKGRFTIS | | | |
| | | | | | | | | | | | | | | | | AAGGGCCGATTCACCATCTCCAGAGAC | | RDNSKNTLYV | | | |
| | | | | | | | | | | | | | | | | AATTCCAAGAACACGCTGTATGTTCAA | | QLNSLRAEDT | | | |
| | | | | | | | | | | | | | | | | CTGAACAGCCTGAGAGCCGAGGACACG | | AVYYCARFHD | | | |
| | | | | | | | | | | | | | | | | GCCGTGTATTACTGTGCGAGATTTCAC | | ISTRYSDAFD | | | |
| | | | | | | | | | | | | | | | | GATATTTCGACTCGTTACTCCGATGCT | | IWGQGTTVTV | | | |
| | | | | | | | | | | | | | | | | TTTGATATCTGGGGCCAAGGGACCACG | | SS | | | |
| | | | | | | | | | | | | | | | | GTCACCGTCTCCTCA | | | | | |
| ADI- | IGHV1- | QVQLVQSG | 150 | YTFTGYQ | 370 | WVRQA | 433 | RINPKS | 707 | RVTLT | 950 | ARGDLN | 1207 | WGQGTT | 1287 | CAGGTGCAGCTGGTGCAGTCTGGGGCT | 1505 | QVQLVQSGAE | 1788 | | |
| 75574 | 2 | AEVKKPGA | | MH | | PGQGL | | GSTNYA | | RDTSI | | I | | VTVSS | | GAGGTGAAGAAGCCTGGGGCCTCAGTG | | VKKPGASVRV | | | |
| | | SVRVSCKA | | | | EWMG | | QKFQG | | TTAYM | | | | | | AGGGTCTCCTGCAAGGCTTCTGAATAC | | SCKASEYTFT | | | |
| | | SE | | | | | | | | ELRRL | | | | | | ACCTTCACCGGCTACCAAATGCACTGG | | GYQMHWVRQA | | | |
| | | | | | | | | | | RSDDT | | | | | | GTGCGACAGGCCCCTGGACAAGGGCTT | | PGQGLEWMGR | | | |
| | | | | | | | | | | AVYYC | | | | | | GAGTGGATGGGACGGATCAACCCTAAG | | INPKSGSTNY | | | |
| | | | | | | | | | | | | | | | | AGTGGTAGCACAAACTATGCACAGAAG | | AQKFQGRVTL | | | |
| | | | | | | | | | | | | | | | | TTTCAGGGCAGGGTCACCTTGACCAGG | | TRDTSITTAY | | | |
| | | | | | | | | | | | | | | | | GACACGTCCATCACCACAGCCTACATG | | MELRRLRSDD | | | |
| | | | | | | | | | | | | | | | | GAGTTGAGGAGGCTGAGATCTGACGAC | | TAVYYCARGD | | | |
| | | | | | | | | | | | | | | | | ACGGCCGTCTATTACTGTGCGAGAGGG | | LNIWGQGTTV | | | |
| | | | | | | | | | | | | | | | | GACCTGAACATCTGGGGCCAAGGGACA | | TVSS | | | |
| | | | | | | | | | | | | | | | | ACGGTCACCGTCTCCTCA | | | | | |
| ADI- | IGHV2- | RSTLKESG | 151 | FSLSTSA | 371 | WIRQP | 441 | LIYWDD | 612 | RLSIT | 951 | AHGQYT | 1208 | LGRGTL | 1301 | AGGTCAACGCTGAAGGAGTCTGGTCCT | 1506 | RSTLKESGPT | 1789 | QITLKESG | 1875 |
| 75620 | 5 | PTLVKPTQ | | VGVG | | PGKAL | | DKRYSP | | KDTSK | | SSWIHW | | VTVSS | | ACGTTGGTAAAACCCACACAGACCCTC | | LVKPTQTLTL | | PTLVKPTQ | |
| | | TLTLTCTF | | | | EWLA | | SLKS | | NQVVL | | YFDL | | | | ACGCTGACCTGCACCTTCTCTGGGTTC | | TCTFSGFSLS | | TLTLTCTF | |
| | | SG | | | | | | | | RMTNM | | | | | | TCACTCAGCACTAGTGCAGTGGGTGTG | | TSAVGVGWIR | | SGFSLSTS | |
| | | | | | | | | | | DPVDT | | | | | | GGCTGGATCCGTCAGCCCCCTGGAAAG | | QPPGKALEWL | | AVGVGWIR | |
| | | | | | | | | | | ATYYC | | | | | | GCCCTGGAGTGGCTTGCACTCATTTAT | | ALIYWDDDKR | | QPPGKALE | |
| | | | | | | | | | | | | | | | | TGGGATGACGATAAGCGCTACAGCCCA | | YSPSLKSRLS | | WLALIYWD | |
| | | | | | | | | | | | | | | | | TCTCTGAAGAGCAGGCTCAGCATCACC | | ITKDTSKNQV | | DDKRYSPS | |
| | | | | | | | | | | | | | | | | AAGGACACCTCCAAAAACCAGGTGGTC | | VLRMTNMDPV | | LKSRLSIT | |
| | | | | | | | | | | | | | | | | CTTAGAATGACCAACATGGACCCTGTG | | DTATYYCAHG | | KDTSKNQV | |
| | | | | | | | | | | | | | | | | GACACAGCCACATATTACTGTGCACAC | | QYTSSWIHWY | | VLRMTNMD | |
| | | | | | | | | | | | | | | | | GGCCAATATACCAGCAGCTGGATCCAT | | FDLLGRGTLV | | PVDTATYY | |
| | | | | | | | | | | | | | | | | TGGTACTTCGATCTCTTGGGCCGTGGC | | TVSS | | CAHGQYTS | |
| | | | | | | | | | | | | | | | | ACCCTGGTCACCGTCTCCTCA | | | | SWIHWYFD | |
| | | | | | | | | | | | | | | | | | | | | LWGRGTLV | |
| | | | | | | | | | | | | | | | | | | | | TVSS | |
| ADI- | IGHV3- | RCSCRQSG | 152 | FTFSNYW | 333 | WVRQT | 500 | RINGDG | 708 | RFTVS | 952 | ARDLES | 1169 | WGQGTL | 1286 | AGGTGCAGCTGCAGGCAGTCTGGGGGA | 1507 | RCSCRQSGGG | 1790 | | |
| 75634 | 74 | GGLVQPGG | | MH | | PGKGL | | SSPTYA | | RDNAK | | GSFHPG | | VTVSS | | GGCTTAGTTCAGCCTGGGGGGTCCCTG | | LVQPGGSLRL | | | |
| | | SLRLSCAA | | | | VWVS | | DSVKG | | NTLYL | | LVRVP | | | | AGACTCTCCTGTGCAGCCTCTGGATTC | | SCAASGFTFS | | | |
| | | SG | | | | | | | | QMNSL | | | | | | ACCTTCAGTAACTACTGGATGCACTGG | | NYWMHWVRQT | | | |
| | | | | | | | | | | RVEDT | | | | | | GTCCGCCAAACTCCAGGGAAGGGGCTG | | PGKGLVWVSR | | | |
| | | | | | | | | | | AVYYC | | | | | | GTCTGGGTCTCACGTATTAATGGAGAT | | INGDGSSPTY | | | |
| | | | | | | | | | | | | | | | | GGGAGTAGTCCGACTTACGCGGACTCC | | ADSVKGRFTV | | | |
| | | | | | | | | | | | | | | | | GTGAAGGGCCGATTCACCGTCTCCAGA | | SRDNAKNTLY | | | |
| | | | | | | | | | | | | | | | | GACAACGCCAAGAACACGCTGTATCTG | | LQMNSLRVED | | | |
| | | | | | | | | | | | | | | | | CAAATGAACAGTCTGAGAGTCGAAGAC | | TAVYYCARDL | | | |
| | | | | | | | | | | | | | | | | ACGGCTGTGTACTACTGTGCAAGAGAC | | ESGSFHPGLV | | | |
| | | | | | | | | | | | | | | | | CTGGAAAGTGGGAGCTTTCATCCCGGC | | RVPWGQGTLV | | | |
| | | | | | | | | | | | | | | | | CTGGTTCGGGTCCCCTGGGGCCAGGGA | | TVSS | | | |
| | | | | | | | | | | | | | | | | ACCCTGGTCACCGTCTCCTCA | | | | | |
| ADI- | IGHV5- | QVQLVQSG | 153 | YSFTSYW | 372 | WVRQM | 476 | IIYPGD | 635 | QVTIS | 953 | ARFDGG | 1209 | WGQGAL | 1285 | CAGGTCCAGCTGGTGCAGTCTGGAGCA | 1508 | QVQLVQSGAE | 1791 | | |
| 75629 | 51 | AEVKKPGQ | | IG | | PGKGL | | SDTRYS | | ADKFI | | SYYDFD | | VTVSS | | GAGGTGAAGAAGCCCGGGCAGTCTCTG | | VKKPGQSLKI | | | |
| | | SLKISCKG | | | | EWMG | | PSFQG | | STAYL | | Y | | | | AAGATCTCCTGTAAGGGTTCTGGATAC | | SCKGSGYSFT | | | |
| | | SG | | | | | | | | QWSSL | | | | | | AGCTTTACCAGCTACTGGATCGGCTGG | | SYWIGWVRQM | | | |
| | | | | | | | | | | KASDT | | | | | | GTGCGCCAGATGCCCGGGAAAGGCCTG | | PGKGLEWMGI | | | |
| | | | | | | | | | | ATYYC | | | | | | GAGTGGATGGGGATCATCTATCCTGGT | | IYPGDSDTRY | | | |
| | | | | | | | | | | | | | | | | GACTCTGATACCAGATACAGCCCGTCC | | SPSFQGQVTI | | | |
| | | | | | | | | | | | | | | | | TTCCAAGGCCAGGTCACCATCTCAGCC | | SADKFISTAY | | | |
| | | | | | | | | | | | | | | | | GACAAGTTCATCAGCACCGCCTACCTG | | LQWSSLKASD | | | |
| | | | | | | | | | | | | | | | | CAGTGGAGCAGCCTGAAGGCCTCGGAC | | TATYYCARFD | | | |
| | | | | | | | | | | | | | | | | ACCGCCACGTATTACTGTGCGAGATTT | | GGSYYDFDYW | | | |
| | | | | | | | | | | | | | | | | GATGGTGGGAGCTACTACGACTTTGAC | | GQGALVTVSS | | | |
| | | | | | | | | | | | | | | | | TACTGGGGCCAGGGAGCCCTGGTCACC | | | | | |
| | | | | | | | | | | | | | | | | GTCTCCTCA | | | | | |
| ADI- | IGHV3- | QVQLQESG | 154 | FPLSYFW | 373 | WVRQT | 500 | RSSSDG | 709 | RFTVS | 954 | AREVSG | 1210 | WGQGTL | 1286 | CAGGTGCAGCTGCAGGAGTCCGGGGGA | 1509 | QVQLQESGGG | 1792 | | |
| 75834 | 74 | GGLVQPGG | | IH | | PGKGL | | SATSYA | | RDNAK | | SGWPFD | | VTVSS | | GGCTTAGTTCAGCCGGGGGGGTCCCTG | | LVQPGGSLRL | | | |
| | | SLRLSCAA | | | | VWVS | | DSVKG | | NTMYL | | S | | | | AGACTCTCCTGTGCAGCCTCTGGATTC | | SCAASGFPLS | | | |
| | | SG | | | | | | | | QMNSL | | | | | | CCCCTCAGTTACTTCTGGATTCACTGG | | YFWIHWVRQT | | | |
| | | | | | | | | | | RVEDT | | | | | | GTCCGCCAAACTCCAGGGAAGGGGCTG | | PGKGLVWVSR | | | |
| | | | | | | | | | | AVYFC | | | | | | GTGTGGGTCTCACGTAGTAGTAGTGAT | | SSSDGSATSY | | | |
| | | | | | | | | | | | | | | | | GGGAGCGCCACAAGCTACGCGGACTCC | | ADSVKGRFTV | | | |
| | | | | | | | | | | | | | | | | GTGAAGGGCCGATTCACCGTCTCCAGA | | SRDNAKNTMY | | | |
| | | | | | | | | | | | | | | | | GACAACGCCAAGAACACGATGTATCTG | | LQMNSLRVED | | | |
| | | | | | | | | | | | | | | | | CAAATGAACAGTCTGAGAGTCGAGGAC | | TAVYFCAREV | | | |
| | | | | | | | | | | | | | | | | ACGGCTGTATACTTTTGTGCAAGAGAG | | SGSGWPFDSW | | | |
| | | | | | | | | | | | | | | | | GTGTCTGGTAGTGGCTGGCCATTTGAC | | GQGTLVTVSS | | | |
| | | | | | | | | | | | | | | | | TCCTGGGGCCAGGGAACCCTGGTCACC | | | | | |
| | | | | | | | | | | | | | | | | GTCTCCTCA | | | | | |
| ADI- | IGHV1- | QVQLVQSG | 50 | GTFRSHS | 374 | WVRQA | 433 | RIIPIL | 710 | RVTIT | 955 | ARVGEM | 1211 | WGQGTT | 1287 | CAGGTCCAGCTGGTGCAGTCTGGGGCT | 1510 | QVQLVQSGAE | 1793 | | |
| 75747 | 69 | AEVKKPGS | | IS | | PGQGL | | GIATYP | | ADKST | | STAGND | | VTVSS | | GAGGTGAAGAAGCCTGGGTCCTCGGTG | | VKKPGSSVKV | | | |
| | | SVKVSCKA | | | | EWMG | | QKFQD | | STAYM | | AFDI | | | | AAGGTCTCCTGCAAGGCTTCTGGAGGC | | SCKASGGTFR | | | |
| | | SG | | | | | | | | ELSSL | | | | | | ACCTTCAGAAGTCATAGTATCAGTTGG | | SHSISWVRQA | | | |
| | | | | | | | | | | ISEDT | | | | | | GTGCGACAGGCCCCTGGACAAGGACTT | | PGQGLEWMGR | | | |
| | | | | | | | | | | AVYFC | | | | | | GAGTGGATGGGAAGGATCATCCCTATC | | IIPILGIATY | | | |
| | | | | | | | | | | | | | | | | CTTGGTATAGCAACCTACCCACAGAAG | | PQKFQDRVTI | | | |
| | | | | | | | | | | | | | | | | TTCCAGGACAGAGTCACGATTACCGCG | | TADKSTSTAY | | | |
| | | | | | | | | | | | | | | | | GACAAATCCACGAGCACAGCCTACATG | | MELSSLISED | | | |
| | | | | | | | | | | | | | | | | GAGCTGAGCAGCCTGATATCTGAGGAC | | TAVYFCARVG | | | |
| | | | | | | | | | | | | | | | | ACGGCCGTGTATTTCTGTGCGAGAGTG | | EMSTAGNDAF | | | |
| | | | | | | | | | | | | | | | | GGGGAGATGTCTACAGCCGGGAATGAT | | DIWGQGTTVT | | | |
| | | | | | | | | | | | | | | | | GCTTTTGATATCTGGGGCCAAGGGACC | | VSS | | | |
| | | | | | | | | | | | | | | | | ACGGTCACCGTCTCCTCA | | | | | |
| ADI- | IGHV1- | RSSWRQSG | 155 | GTFSNHF | 375 | WVRQA | 433 | RIIPVL | 711 | RVTIT | 956 | ARGSGY | 1212 | WGPGTL | 1302 | AGGTCCAGCTGGAGGCAGTCTGGGGCT | 1511 | RSSWRQSGAE | 1794 | | |
| 75648 | 69 | AEVKKPGS | | IS | | PGQGL | | GIADYA | | ADKST | | SGSGSN | | VTVSS | | GAGGTGAAGAAGCCTGGGTCCTCGGTG | | VKKPGSSVKV | | | |
| | | SVKVSCRA | | | | EWMG | | QKFQG | | STAYL | | YYFDP | | | | AAGGTCTCCTGCAGGGCTTCTGGAGGC | | SCRASGGTFS | | | |
| | | SG | | | | | | | | ELSSL | | | | | | ACCTTCAGCAATCATTTTATCAGCTGG | | NHFISWVRQA | | | |
| | | | | | | | | | | KSEDT | | | | | | GTGCGACAGGCCCCTGGACAAGGCCTT | | PGQGLEWMGR | | | |
| | | | | | | | | | | AIYFC | | | | | | GAGTGGATGGGAAGGATCATCCCTGTC | | IIPVLGIADY | | | |
| | | | | | | | | | | | | | | | | CTTGGTATAGCAGACTACGCACAGAAG | | AQKFQGRVTI | | | |
| | | | | | | | | | | | | | | | | TTCCAGGGCAGAGTCACGATTACCGCG | | TADKSTSTAY | | | |
| | | | | | | | | | | | | | | | | GACAAATCCACGAGCACAGCCTACCTG | | LELSSLKSED | | | |
| | | | | | | | | | | | | | | | | GAGCTGAGCAGCCTGAAATCTGAAGAC | | TAIYFCARGS | | | |
| | | | | | | | | | | | | | | | | ACGGCCATTTATTTCTGTGCGCGTGGT | | GYSGSGSNYY | | | |
| | | | | | | | | | | | | | | | | TCGGGGTACTCTGGTTCAGGGAGTAAC | | FDPWGPGTLV | | | |
| | | | | | | | | | | | | | | | | TACTACTTCGACCCCTGGGGCCCGGGA | | TVSS | | | |
| | | | | | | | | | | | | | | | | ACCCTGGTCACCGTCTCCTCA | | | | | |
| ADI- | IGHV2- | QVQLQESG | 156 | FSLNSAG | 376 | WVRQP | 501 | LIYWDD | 712 | RLAIT | 957 | ARHSIT | 1213 | WGPGTL | 1302 | CAGGTGCAGCTGCAGGAGTCCGGTCCT | 1512 | QVQLQESGPT | 1795 | | |
| 75862 | 5 | PTLVKPTQ | | VGVG | | PGKAL | | EKRYSP | | KDTSK | | TIFDY | | VTVSS | | ACGCTGGTGAAACCCACACAGACCCTC | | LVKPTQTLTL | | | |
| | | TLTLTCTF | | | | EWLA | | PLKT | | NQVVL | | | | | | ACGCTGACCTGCACCTTCTCTGGGTTC | | TCTFSGFSLN | | | |
| | | SG | | | | | | | | RMTNM | | | | | | TCGCTCAACTCTGCTGGAGTGGGTGTG | | SAGVGVGWVR | | | |
| | | | | | | | | | | DPVDT | | | | | | GGCTGGGTCCGCCAGCCCCCAGGAAAG | | QPPGKALEWL | | | |
| | | | | | | | | | | ATYYC | | | | | | GCCCTGGAGTGGCTTGCTCTCATTTAT | | ALIYWDDEKR | | | |
| | | | | | | | | | | | | | | | | TGGGATGATGAGAAGCGCTACAGCCCA | | YSPPLKTRLA | | | |
| | | | | | | | | | | | | | | | | CCTCTGAAGACCAGACTCGCCATCACC | | ITKDTSKNQV | | | |
| | | | | | | | | | | | | | | | | AAGGACACCTCCAAAAACCAGGTGGTC | | VLRMTNMDPV | | | |
| | | | | | | | | | | | | | | | | CTTAGAATGACCAACATGGACCCTGTG | | DTATYYCARH | | | |
| | | | | | | | | | | | | | | | | GACACAGCCACATATTATTGTGCACGT | | SITTIFDYWG | | | |
| | | | | | | | | | | | | | | | | CACTCGATTACGACGATCTTTGACTAC | | PGTLVTVSS | | | |
| | | | | | | | | | | | | | | | | TGGGGCCCGGGAACCCTGGTCACCGTC | | | | | |
| | | | | | | | | | | | | | | | | TCCTCA | | | | | |
| ADI- | IGHV1- | QVQLVQSG | 135 | YAFSNYN | 377 | WVRQA | 433 | IIYPTG | 713 | RVTMT | 958 | AREGVT | 1214 | WGQGAL | 1285 | CAGGTGCAGCTGGTGCAGTCTGGGGCT | 1513 | QVQLVQSGAE | 1796 | | |
| 75693 | 46 | AEVKKPGA | | LH | | PGQGL | | GGTRSA | | RDTST | | GTGYFD | | VTVSS | | GAGGTGAAAAAGCCTGGGGCCTCAGTG | | VKKPGASVRV | | | |
| | | SVRVSCKA | | | | EWMG | | QSVQG | | STIYL | | Y | | | | AGGGTTTCCTGCAAGGCCTCTGGATAT | | SCKASGYAFS | | | |
| | | SG | | | | | | | | DLSSL | | | | | | GCCTTCAGCAACTACAATCTGCACTGG | | NYNLHWVRQA | | | |
| | | | | | | | | | | RSEDT | | | | | | GTGCGACAGGCCCCTGGACAGGGGCTT | | PGQGLEWMGI | | | |
| | | | | | | | | | | AMYYC | | | | | | GAGTGGATGGGAATAATCTACCCTACT | | IYPTGGGTRS | | | |
| | | | | | | | | | | | | | | | | GGAGGTGGCACACGGTCCGCACAGAGT | | AQSVQGRVTM | | | |
| | | | | | | | | | | | | | | | | GTCCAGGGCAGAGTCACCATGACCAGG | | TRDTSTSTIY | | | |
| | | | | | | | | | | | | | | | | GACACGTCCACGAGCACAATTTACCTG | | LDLSSLRSED | | | |
| | | | | | | | | | | | | | | | | GACTTGAGCAGCCTGAGATCTGAGGAC | | TAMYYCAREG | | | |
| | | | | | | | | | | | | | | | | ACGGCCATGTATTATTGTGCTAGAGAG | | VTGTGYFDYW | | | |
| | | | | | | | | | | | | | | | | GGAGTGACTGGGACGGGCTACTTTGAC | | GQGALVTVSS | | | |
| | | | | | | | | | | | | | | | | TACTGGGGCCAGGGAGCCCTGGTCACC | | | | | |
| | | | | | | | | | | | | | | | | GTCTCCTCA | | | | | |
| ADI- | IGHV5- | QVQLQQSG | 157 | YSFTNYW | 378 | WVRQK | 502 | RIDPSD | 714 | HVTIS | 959 | ARHLRV | 1215 | WGQGTL | 1286 | CAGGTCCAGCTGCAGCAGTCCGGAGCA | 1514 | QVQLQQSGAE | 1797 | | |
| 75842 | 10-1 | AEVKKPGE | | IT | | PGQGL | | SYSNYS | | ADKSI | | VPGYYL | | VTVSS | | GAGGTGAAAAAGCCCGGGGAGTCTCTG | | VKKPGESLRI | | | |
| | | SLRISCKG | | | | EWMG | | PSFHG | | STAYL | | FDN | | | | AGGATCTCCTGTAAGGGCTCTGGATAC | | SCKGSGYSFT | | | |
| | | SG | | | | | | | | QWSSL | | | | | | AGCTTTACCAATTACTGGATCACCTGG | | NYWITWVRQK | | | |
| | | | | | | | | | | QASDT | | | | | | GTGCGCCAGAAGCCCGGGCAAGGCCTG | | PGQGLEWMGR | | | |
| | | | | | | | | | | AIYYC | | | | | | GAGTGGATGGGGAGGATTGATCCTAGT | | IDPSDSYSNY | | | |
| | | | | | | | | | | | | | | | | GACTCTTACAGCAACTACAGCCCGTCC | | SPSFHGHVTI | | | |
| | | | | | | | | | | | | | | | | TTCCACGGCCACGTCACCATCTCAGCT | | SADKSISTAY | | | |
| | | | | | | | | | | | | | | | | GACAAGTCCATCAGCACTGCCTACCTG | | LQWSSLQASD | | | |
| | | | | | | | | | | | | | | | | CAGTGGAGCAGCCTGCAGGCCTCGGAC | | TAIYYCARHL | | | |
| | | | | | | | | | | | | | | | | ACCGCCATCTATTATTGTGCGAGACAT | | RVVPGYYLFD | | | |
| | | | | | | | | | | | | | | | | CTACGGGTAGTACCAGGCTACTACCTC | | NWGQGTLVTV | | | |
| | | | | | | | | | | | | | | | | TTTGACAACTGGGGCCAGGGAACCCTG | | SS | | | |
| | | | | | | | | | | | | | | | | GTCACCGTCTCCTCA | | | | | |
| ADI- | IGHV3- | QVQLVESG | 100 | FTFDDYA | 214 | WVRQA | 437 | VISWNS | 715 | RFTIS | 960 | AREGCR | 1216 | WGQGTL | 1286 | CAGGTGCAGCTGGTGGAGTCTGGGGGA | 1515 | QVQLVESGGG | 1798 | | |
| 75828 | 9 | GGLVQPGR | | MH | | PGKGL | | DSIGYA | | RDNAK | | STTCYD | | VTVSS | | GGCTTGGTACAGCCTGGCAGGTCCCTG | | LVQPGRSLRL | | | |
| | | SLRLSCAA | | | | EWVS | | DSVKG | | NSFYL | | Y | | | | AGACTCTCCTGTGCAGCCTCTGGATTC | | SCAASGFTFD | | | |
| | | SG | | | | | | | | QMNSL | | | | | | ACCTTTGATGATTACGCCATGCACTGG | | DYAMHWVRQA | | | |
| | | | | | | | | | | RPEDT | | | | | | GTCCGGCAAGCTCCAGGGAAGGGCCTG | | PGKGLEWVSV | | | |
| | | | | | | | | | | ALYYC | | | | | | GAGTGGGTCTCCGTTATTAGTTGGAAT | | ISWNSDSIGY | | | |
| | | | | | | | | | | | | | | | | AGTGATAGCATAGGCTATGCGGACTCT | | ADSVKGRFTI | | | |
| | | | | | | | | | | | | | | | | GTGAAGGGCCGATTCACCATCTCCAGA | | SRDNAKNSFY | | | |
| | | | | | | | | | | | | | | | | GACAACGCCAAGAACTCCTTCTATCTG | | LQMNSLRPED | | | |
| | | | | | | | | | | | | | | | | CAAATGAACAGTCTGAGACCTGAGGAC | | TALYYCAREG | | | |
| | | | | | | | | | | | | | | | | ACGGCCTTGTATTACTGTGCAAGGGAG | | CRSTTCYDYW | | | |
| | | | | | | | | | | | | | | | | GGTTGTAGAAGTACCACCTGCTATGAC | | GQGTLVTVSS | | | |
| | | | | | | | | | | | | | | | | TACTGGGGCCAGGGAACCCTGGTCACC | | | | | |
| | | | | | | | | | | | | | | | | GTCTCCTCA | | | | | |
| ADI- | IGHV1- | QVQLQQSG | 86 | GTFSSYP | 379 | WVRQA | 433 | SIIPIL | 716 | RVTIN | 961 | ARDLGY | 1217 | WGQGTL | 1286 | CAGGTACAGCTGCAGCAGTCTGGGGCT | 1516 | QVQLQQSGAE | 1799 | | |
| 75632 | 69 | AEVKKPGS | | IS | | PGQGL | | GVAKYA | | ADKST | | SSGGAN | | VTVSS | | GAGGTGAAGAAGCCTGGGTCCTCGGTG | | VKKPGSSVKV | | | |
| | | SVKVSCKA | | | | EWMG | | QKFQG | | STAYM | | YYFDY | | | | AAGGTCTCCTGCAAGGCTTCTGGAGGC | | SCKASGGTFS | | | |
| | | ISG | | | | | | | | ELSSL | | | | | | ACCTTCAGCAGCTATCCTATCAGCTGG | | SYPISWVRQA | | | |
| | | | | | | | | | | RFEDT | | | | | | GTGCGACAGGCCCCTGGACAAGGGCTT | | PGQGLEWMGS | | | |
| | | | | | | | | | | AVYYC | | | | | | GAGTGGATGGGAAGTATCATCCCTATC | | IIPILGVAKY | | | |
| | | | | | | | | | | | | | | | | CTTGGTGTCGCAAAATACGCACAGAAG | | AQKFQGRVTI | | | |
| | | | | | | | | | | | | | | | | TTCCAGGGCAGAGTCACGATTAACGCG | | NADKSTSTAY | | | |
| | | | | | | | | | | | | | | | | GACAAATCCACGAGCACAGCCTACATG | | MELSSLRFED | | | |
| | | | | | | | | | | | | | | | | GAGCTGAGCAGCCTGAGATTTGAGGAC | | TAVYYCARDL | | | |
| | | | | | | | | | | | | | | | | ACGGCCGTGTATTACTGTGCGAGAGAT | | GYSSGGANYY | | | |
| | | | | | | | | | | | | | | | | TTGGGGTATAGCAGTGGCGGGGCCAAT | | FDYWGQGTLV | | | |
| | | | | | | | | | | | | | | | | TACTACTTTGACTACTGGGGCCAGGGA | | TVSS | | | |
| | | | | | | | | | | | | | | | | ACCCTGGTCACCGTCTCCTCA | | | | | |
| ADI- | IGHV3- | QVQLVESG | 158 | LTVSSNY | 380 | WVRQA | 437 | VIYSGG | 659 | RFTIS | 777 | ARMPYG | 1218 | WGQGTT | 1287 | CAGGTGCAGCTGGTGGAGTCTGGAGGA | 1517 | QVQLVESGGG | 1800 | | |
| 75855 | 53 | GGLIQPGG | | MS | | PGKGL | | STYYAD | | RDNSK | | TDV | | VTVSS | | GGCTTGATCCAGCCTGGGGGGTCCCTG | | LIQPGGSLRL | | | |
| | | SLRLSCEA | | | | EWVS | | SVKG | | NTLYL | | | | | | AGACTCTCCTGTGAAGCCTCTGGGCTC | | SCEASGLTVS | | | |
| | | SG | | | | | | | | QMNSL | | | | | | ACCGTCAGTAGCAACTACATGAGCTGG | | SNYMSWVRQA | | | |
| | | | | | | | | | | RAEDT | | | | | | GTCCGCCAGGCTCCAGGGAAGGGGCTG | | PGKGLEWVSV | | | |
| | | | | | | | | | | AVYYC | | | | | | GAGTGGGTCTCAGTTATTTATAGCGGT | | IYSGGSTYYA | | | |
| | | | | | | | | | | | | | | | | GGTAGCACATACTACGCAGACTCCGTG | | DSVKGRFTIS | | | |
| | | | | | | | | | | | | | | | | AAGGGCCGATTCACCATCTCCAGAGAC | | RDNSKNTLYL | | | |
| | | | | | | | | | | | | | | | | AATTCCAAGAACACGCTGTATCTTCAA | | QMNSLRAEDT | | | |
| | | | | | | | | | | | | | | | | ATGAACAGCCTGAGAGCCGAGGACACG | | AVYYCARMPY | | | |
| | | | | | | | | | | | | | | | | GCCGTGTATTACTGTGCGAGAATGCCT | | GTDVWGQGTT | | | |
| | | | | | | | | | | | | | | | | TACGGTACGGACGTCTGGGGCCAAGGG | | VTVSS | | | |
| | | | | | | | | | | | | | | | | ACCACGGTCACCGTCTCCTCA | | | | | |
| ADI- | IGHV4- | QVQLQQSG | 56 | GSISRGS | 381 | WIRQH | 450 | YIYSSG | 717 | RLTIS | 962 | ARHSVP | 1219 | WGQGNL | 1303 | CAGGTGCAGCTGCAGCAGTCGGGCCCA | 1518 | QVQLQQSGPG | 1801 | | |
| 75729 | 31 | PGLVKPSQ | | YYWS | | PGKGL | | STYYNP | | LDTSK | | YYFDY | | VTVSS | | GGACTGGTGAAGCCTTCACAGACCCTG | | LVKPSQTLSL | | | |
| | | TLSLTCTV | | | | EWIG | | SLES | | NQFSL | | | | | | TCCCTCACCTGCACTGTCTCTGGTGGC | | TCTVSGGSIS | | | |
| | | SG | | | | | | | | RLTSV | | | | | | TCCATCAGCAGGGGTTCTTACTACTGG | | RGSYYWSWIR | | | |
| | | | | | | | | | | TAADT | | | | | | AGCTGGATCCGCCAGCACCCAGGGAAG | | QHPGKGLEWI | | | |
| | | | | | | | | | | AVYFC | | | | | | GGCCTGGAGTGGATTGGTTACATCTAT | | GYIYSSGSTY | | | |
| | | | | | | | | | | | | | | | | TCCAGTGGGAGCACCTACTACAACCCG | | YNPSLESRLT | | | |
| | | | | | | | | | | | | | | | | TCCCTCGAGAGTCGACTTACCATATCA | | ISLDTSKNQF | | | |
| | | | | | | | | | | | | | | | | TTAGACACGTCTAAGAACCAGTTCTCC | | SLRLTSVTAA | | | |
| | | | | | | | | | | | | | | | | CTGAGGCTGACCTCTGTGACTGCCGCG | | DTAVYFCARH | | | |
| | | | | | | | | | | | | | | | | GACACGGCCGTCTATTTCTGTGCGAGG | | SVPYYFDYWG | | | |
| | | | | | | | | | | | | | | | | CATTCTGTGCCGTACTACTTTGACTAC | | QGNLVTVSS | | | |
| | | | | | | | | | | | | | | | | TGGGGCCAGGGAAACCTGGTCACCGTC | | | | | |
| | | | | | | | | | | | | | | | | TCCTCA | | | | | |
| ADI- | IGHV7- | QVQLVQSG | 159 | YTFNTYA | 382 | WVRQA | 433 | WINTNT | 718 | RFVFS | 963 | ARDEES | 1220 | WGQGTL | 1286 | CAGGTCCAGCTGGTGCAGTCTGGGTCT | 1519 | QVQLVQSGSE | 1802 | | |
| 75734 | 4-1 | SELKKPGA | | MN | | PGQGL | | GATTYA | | FDTSL | | LYEAVT | | VTVSS | | GAGTTGAAGAAGCCTGGGGCCTCAGTG | | LKKPGASVKV | | | |
| | | SVKVSCKA | | | | EWMG | | QGFTG | | TTAYL | | GTGWFD | | | | AAGGTTTCCTGCAAGGCTTCTGGATAT | | SCKASGYTFN | | | |
| | | SG | | | | | | | | QISSL | | P | | | | ACCTTCAATACCTACGCTATGAATTGG | | TYAMNWVRQA | | | |
| | | | | | | | | | | RAEDT | | | | | | GTGCGACAGGCCCCTGGACAAGGGCTT | | PGQGLEWMGW | | | |
| | | | | | | | | | | AVYFC | | | | | | GAGTGGATGGGATGGATCAACACCAAC | | INTNTGATTY | | | |
| | | | | | | | | | | | | | | | | ACTGGGGCCACAACGTATGCCCAGGGC | | AQGFTGRFVF | | | |
| | | | | | | | | | | | | | | | | TTCACAGGGCGTTTTGTCTTCTCCTTC | | SFDTSLTTAY | | | |
| | | | | | | | | | | | | | | | | GACACCTCTCTCACTACGGCATATCTG | | LQISSLRAED | | | |
| | | | | | | | | | | | | | | | | CAGATCAGCAGCCTAAGGGCTGAGGAC | | TAVYFCARDE | | | |
| | | | | | | | | | | | | | | | | ACTGCCGTGTATTTCTGTGCGAGAGAT | | ESLYEAVTGT | | | |
| | | | | | | | | | | | | | | | | GAGGAGTCGCTCTATGAAGCAGTGACT | | GWFDPWGQGT | | | |
| | | | | | | | | | | | | | | | | GGTACAGGCTGGTTCGACCCCTGGGGC | | LVTVSS | | | |
| | | | | | | | | | | | | | | | | CAGGGAACCCTGGTCACCGTCTCCTCA | | | | | |
| ADI- | IGHV3- | QVTLKESG | 160 | ADFSTYG | 383 | WVRQA | 431 | ALSHDG | 719 | RFTIS | 964 | AKDLIN | 1221 | WGQGTL | 1286 | CAGGTCACGCTGAAGGAGTCTGGGGGA | 1520 | QVTLKESGGG | 1803 | | |
| 75880 | 30 | GGVVQPGT | | MH | | PGKGL | | AYKFYA | | RDNSK | | YDPWSG | | VTVSS | | GGCGTGGTCCAGCCTGGGACGTCCCAG | | VVQPGTSQRV | | | |
| | | SQRVSCTV | | | | EWVA | | DSVKG | | KTAFL | | FFT | | | | AGAGTCTCCTGCACAGTCTCTGGAGCC | | SCTVSGADFS | | | |
| | | SG | | | | | | | | QMNSV | | | | | | GACTTCAGTACTTACGGCATGCACTGG | | TYGMHWVRQA | | | |
| | | | | | | | | | | RPEDT | | | | | | GTCCGCCAGGCTCCAGGCAAGGGGCTG | | PGKGLEWVAA | | | |
| | | | | | | | | | | AVYYC | | | | | | GAGTGGGTGGCAGCTCTCTCACATGAT | | LSHDGAYKFY | | | |
| | | | | | | | | | | | | | | | | GGAGCTTATAAATTCTATGCAGACTCA | | ADSVKGRFTI | | | |
| | | | | | | | | | | | | | | | | GTGAAGGGCCGATTCACCATCTCCAGA | | SRDNSKKTAF | | | |
| | | | | | | | | | | | | | | | | GACAATTCCAAGAAAACGGCGTTTCTG | | LQMNSVRPED | | | |
| | | | | | | | | | | | | | | | | CAAATGAACAGTGTGAGACCGGAGGAC | | TAVYYCAKDL | | | |
| | | | | | | | | | | | | | | | | ACGGCTGTCTATTACTGTGCGAAGGAC | | INYDPWSGFF | | | |
| | | | | | | | | | | | | | | | | CTGATTAACTACGATCCCTGGAGTGGC | | TWGQGTLVTV | | | |
| | | | | | | | | | | | | | | | | TTTTTCACTTGGGGCCAGGGAACCCTG | | SS | | | |
| | | | | | | | | | | | | | | | | GTCACCGTCTCCTCA | | | | | |
| ADI- | IGHV5- | QVQLVQSG | 161 | YSFSNYW | 384 | WVRQM | 476 | IIYPGD | 635 | HMTIS | 965 | ARQGTT | 1222 | WGQGTL | 1286 | CAGGTCCAGCTTGTGCAGTCTGGAGCA | 1521 | QVQLVQSGAE | 1804 | | |
| 75724 | 51 | AEVKKSGE | | IG | | PGKGL | | SDTRYS | | ADKSI | | VGHFDN | | VTVSS | | GAGGTGAAAAAGTCCGGGGAGTCTCTG | | VKKSGESLKI | | | |
| | | SLKISCKG | | | | EWMG | | PSFQG | | STAYL | | | | | | AAGATCTCCTGCAAGGGTTCTGGATAC | | SCKGSGYSFS | | | |
| | | SG | | | | | | | | QWNSL | | | | | | AGCTTTAGCAACTACTGGATCGGCTGG | | NYWIGWVRQM | | | |
| | | | | | | | | | | KASDT | | | | | | GTGCGCCAGATGCCCGGGAAAGGCCTG | | PGKGLEWMGI | | | |
| | | | | | | | | | | AMYYC | | | | | | GAGTGGATGGGGATCATCTATCCTGGT | | IYPGDSDTRY | | | |
| | | | | | | | | | | | | | | | | GACTCTGACACCAGATACAGCCCGTCC | | SPSFQGHMTI | | | |
| | | | | | | | | | | | | | | | | TTCCAAGGCCACATGACCATCTCAGCC | | SADKSISTAY | | | |
| | | | | | | | | | | | | | | | | GACAAGTCCATCAGTACCGCCTACCTG | | LQWNSLKASD | | | |
| | | | | | | | | | | | | | | | | CAGTGGAACAGCCTGAAGGCCTCGGAC | | TAMYYCARQG | | | |
| | | | | | | | | | | | | | | | | ACCGCCATGTATTACTGTGCGAGACAG | | TTVGHFDNWG | | | |
| | | | | | | | | | | | | | | | | GGAACGACAGTAGGGCACTTTGACAAC | | QGTLVTVSS | | | |
| | | | | | | | | | | | | | | | | TGGGGCCAGGGAACCCTGGTCACCGTC | | | | | |
| | | | | | | | | | | | | | | | | TCCTCA | | | | | |
| ADI- | IGHV1- | QVQLVQSG | 50 | GTFSSYT | 385 | WVRQA | 433 | RIFPIL | 720 | RVTIS | 966 | ARDIGY | 1223 | WGQGTL | 1286 | CAGGTGCAGCTGGTGCAGTCTGGGGCT | 1522 | QVQLVQSGAE | 1805 | | |
| 75737 | 69 | AEVKKPGS | | FS | | PGQGL | | GVANYA | | ADKST | | SDYGAT | | VTVSS | | GAAGTGAAGAAGCCTGGGTCCTCGGTG | | VKKPGSSVKV | | | |
| | | SVKVSCKA | | | | EWMG | | QKFQG | | TTAYM | | YWFDP | | | | AAGGTCTCCTGCAAGGCTTCTGGAGGC | | SCKASGGTFS | | | |
| | | SG | | | | | | | | ELSSL | | | | | | ACCTTCAGCAGCTACACTTTCAGCTGG | | SYTFSWVRQA | | | |
| | | | | | | | | | | TSEDT | | | | | | GTGCGACAGGCCCCTGGACAAGGGCTT | | PGQGLEWMGR | | | |
| | | | | | | | | | | AVYYC | | | | | | GAGTGGATGGGAAGGATCTTCCCTATC | | IFPILGVANY | | | |
| | | | | | | | | | | | | | | | | CTTGGTGTAGCAAACTACGCACAGAAG | | AQKFQGRVTI | | | |
| | | | | | | | | | | | | | | | | TTCCAGGGCAGAGTCACCATTTCCGCG | | SADKSTTTAY | | | |
| | | | | | | | | | | | | | | | | GACAAATCAACGACCACGGCCTACATG | | MELSSLTSED | | | |
| | | | | | | | | | | | | | | | | GAACTGAGCAGCTTGACATCTGAGGAC | | TAVYYCARDI | | | |
| | | | | | | | | | | | | | | | | ACGGCCGTCTATTATTGTGCGAGAGAT | | GYSDYGATYW | | | |
| | | | | | | | | | | | | | | | | ATCGGATACAGTGACTACGGGGCAACA | | FDPWGQGTLV | | | |
| | | | | | | | | | | | | | | | | TACTGGTTCGACCCCTGGGGCCAGGGA | | TVSS | | | |
| | | | | | | | | | | | | | | | | ACCCTGGTCACCGTCTCCTCA | | | | | |
| ADI- | IGHV3- | QVQLQQSG | 162 | FTFDDYA | 214 | WVRQA | 437 | GVTWNS | 721 | RFTIS | 967 | VKDRYN | 1224 | WGQGTT | 1287 | CAGGTGCAGCTGCAGCAGTCCGGGGGA | 1523 | QVQLQQSGGG | 1806 | | |
| 75610 | 9 | GGLVQPGR | | MH | | PGKGL | | GTIGYA | | RDNAK | | WKREYY | | VTVSS | | GGCTTGGTACAGCCTGGCAGGTCCCTG | | LVQPGRSLRL | | | |
| | | SLRLSCAA | | | | EWVS | | DSVKG | | NSLYL | | YGMDV | | | | AGACTCTCCTGTGCAGCCTCCGGATTC | | SCAASGFTFD | | | |
| | | SG | | | | | | | | HMRSL | | | | | | ACCTTTGATGATTATGCCATGCACTGG | | DYAMHWVRQA | | | |
| | | | | | | | | | | TPEDT | | | | | | GTCCGGCAAGCTCCAGGGAAGGGCCTG | | PGKGLEWVSG | | | |
| | | | | | | | | | | ALYYC | | | | | | GAGTGGGTCTCAGGTGTTACTTGGAAT | | VTWNSGTIGY | | | |
| | | | | | | | | | | | | | | | | AGTGGTACCATAGGTTACGCGGACTCT | | ADSVKGRFTI | | | |
| | | | | | | | | | | | | | | | | GTGAAGGGCCGATTCACCATCTCCAGA | | SRDNAKNSLY | | | |
| | | | | | | | | | | | | | | | | GACAACGCCAAGAACTCCCTATATCTG | | LHMRSLTPED | | | |
| | | | | | | | | | | | | | | | | CACATGCGCAGTTTGACACCTGAGGAC | | TALYYCVKDR | | | |
| | | | | | | | | | | | | | | | | ACGGCCCTTTATTACTGTGTAAAAGAT | | YNWKREYYYG | | | |
| | | | | | | | | | | | | | | | | CGCTATAACTGGAAAAGGGAGTACTAC | | MDVWGQGTTV | | | |
| | | | | | | | | | | | | | | | | TACGGTATGGACGTCTGGGGCCAAGGG | | TVSS | | | |
| | | | | | | | | | | | | | | | | ACCACGGTCACCGTCTCCTCA | | | | | |
| ADI- | IGHV3- | RCTLEESG | 163 | FTFSNFG | 386 | WVRQA | 431 | AISFDG | 722 | RFTIS | 968 | GKDLDI | 1225 | WGQGTT | 1287 | AGATGCACGCTGGAGGAGTCTGGGGGA | 1524 | RCTLEESGGG | 1807 | | |
| 75603 | 30 | GGVVQPGR | | MH | | PGKGL | | DITYYA | | RDDSK | | VVAVVG | | VTVSS | | GGCGTGGTCCAGCCTGGGAGGTCCCTG | | VVQPGRSLRL | | | |
| | | SLRLSCEA | | | | EWVA | | DSVKG | | NTLHL | | TPYYYY | | | | AGACTCTCTTGTGAAGCCTCTGGATTC | | SCEASGFTFS | | | |
| | | SG | | | | | | | | QIHSL | | GLDV | | | | ACCTTTAGTAATTTTGGCATGCACTGG | | NFGMHWVRQA | | | |
| | | | | | | | | | | RLEDT | | | | | | GTCCGCCAGGCTCCAGGCAAGGGGCTG | | PGKGLEWVAA | | | |
| | | | | | | | | | | ATYYC | | | | | | GAGTGGGTGGCAGCTATATCATTTGAT | | ISFDGDITYY | | | |
| | | | | | | | | | | | | | | | | GGAGATATCACATACTATGCAGACTCC | | ADSVKGRFTI | | | |
| | | | | | | | | | | | | | | | | GTGAAGGGCCGATTCACCATCTCCAGA | | SRDDSKNTLH | | | |
| | | | | | | | | | | | | | | | | GACGATTCCAAGAACACTCTGCATCTC | | LQIHSLRLED | | | |
| | | | | | | | | | | | | | | | | CAAATACACAGCCTGAGACTTGAGGAC | | TATYYCGKDL | | | |
| | | | | | | | | | | | | | | | | ACGGCTACTTATTACTGTGGGAAAGAT | | DIVVAVVGTP | | | |
| | | | | | | | | | | | | | | | | CTCGATATTGTGGTGGCGGTAGTTGGC | | YYYYGLDVWG | | | |
| | | | | | | | | | | | | | | | | ACCCCTTATTACTATTACGGTCTGGAC | | QGTTVTVSS | | | |
| | | | | | | | | | | | | | | | | GTCTGGGGCCAAGGGACCACGGTCACC | | | | | |
| | | | | | | | | | | | | | | | | GTCTCCTCA | | | | | |
| ADI- | IGHV3- | QVTLKESG | 164 | FTVSRFA | 387 | WVRQA | 431 | MISFDG | 723 | RFTIS | 969 | ARGDYY | 1226 | WGQGTL | 1286 | CAGGTCACGTTGAAGGAGTCTGGGGGA | 1525 | QVTLKESGGG | 1808 | | |
| 75649 | 30 | GGVVQPGR | | MH | | PGKGL | | SEKYYA | | RDNSK | | GSGSSY | | VTVSS | | GGCGTGGTCCAGCCTGGGAGGTCCCTG | | VVQPGRSLRL | | | |
| | | SLRLSCAV | | | | EWVA | | DSVKG | | ITLYL | | NPLPTF | | | | AGACTCTCCTGTGCAGTCTCTGGATTC | | SCAVSGFTVS | | | |
| | | SG | | | | | | | | QMNSL | | DY | | | | ACTGTCAGTAGATTTGCTATGCACTGG | | RFAMHWVRQA | | | |
| | | | | | | | | | | RAEDT | | | | | | GTCCGCCAGGCTCCAGGCAAGGGGCTG | | PGKGLEWVAM | | | |
| | | | | | | | | | | AMYYC | | | | | | GAGTGGGTGGCAATGATATCATTTGAT | | ISFDGSEKYY | | | |
| | | | | | | | | | | | | | | | | GGAAGTGAAAAATACTACGCAGACTCC | | ADSVKGRFTI | | | |
| | | | | | | | | | | | | | | | | GTGAAGGGCCGATTCACCATCTCCAGA | | SRDNSKITLY | | | |
| | | | | | | | | | | | | | | | | GACAACTCCAAGATCACACTGTATCTA | | LQMNSLRAED | | | |
| | | | | | | | | | | | | | | | | CAAATGAACAGCCTGAGAGCTGAGGAC | | TAMYYCARGD | | | |
| | | | | | | | | | | | | | | | | ACGGCTATGTATTACTGTGCGAGAGGA | | YYGSGSSYNP | | | |
| | | | | | | | | | | | | | | | | GATTACTATGGTTCAGGGAGTTCCTAT | | LPTFDYWGQG | | | |
| | | | | | | | | | | | | | | | | AATCCACTGCCCACCTTTGACTACTGG | | TLVTVSS | | | |
| | | | | | | | | | | | | | | | | GGCCAGGGAACCCTGGTCACCGTCTCC | | | | | |
| | | | | | | | | | | | | | | | | TCA | | | | | |
| ADI- | IGHV3- | QVQLVQSG | 165 | FTFSSYT | 388 | WVRQA | 503 | FISHDG | 724 | RFTIS | 777 | ARGDIL | 1227 | WGHGTT | 1304 | CAGGTGCAGCTGGTGCAGTCTGGGGGA | 1526 | QVQLVQSGGG | 1809 | | |
| 75625 | 30 | GGVVQPGR | | LH | | PGEGL | | GRIDYA | | RDNSK | | TGYPIS | | VTVSS | | GGCGTGGTCCAGCCTGGGAGGTCCCTG | | VVQPGRSLRL | | | |
| | | SLRLSCAA | | | | EWVS | | DSVKG | | NTLYL | | KDALDL | | | | AGACTCTCCTGTGCAGCGTCTGAATTC | | SCAASEFTFS | | | |
| | | SE | | | | | | | | QMNSL | | | | | | ACCTTCAGTAGTTATACTCTGCACTGG | | SYTLHWVRQA | | | |
| | | | | | | | | | | RAEDT | | | | | | GTCCGCCAGGCTCCAGGCGAGGGACTG | | PGEGLEWVSF | | | |
| | | | | | | | | | | AVYYC | | | | | | GAGTGGGTGTCATTTATATCACATGAT | | ISHDGGRIDY | | | |
| | | | | | | | | | | | | | | | | GGAGGTAGAATAGACTACGCAGACTCC | | ADSVKGRFTI | | | |
| | | | | | | | | | | | | | | | | GTGAAGGGCCGATTCACCATCTCCAGA | | SRDNSKNTLY | | | |
| | | | | | | | | | | | | | | | | GACAATTCCAAGAACACGCTGTATCTG | | LQMNSLRAED | | | |
| | | | | | | | | | | | | | | | | CAAATGAACAGCCTGAGAGCTGAGGAC | | TAVYYCARGD | | | |
| | | | | | | | | | | | | | | | | ACGGCTGTGTATTACTGTGCGAGAGGC | | ILTGYPISKD | | | |
| | | | | | | | | | | | | | | | | GACATTTTGACTGGGTACCCGATATCA | | ALDLWGHGTT | | | |
| | | | | | | | | | | | | | | | | AAGGATGCTCTTGATCTCTGGGGCCAC | | VTVSS | | | |
| | | | | | | | | | | | | | | | | GGGACCACGGTCACCGTCTCCTCA | | | | | |
| ADI- | IGHV3- | QVQLVQSG | 166 | FTFNSYW | 389 | WVRQA | 431 | SIKQDG | 725 | RFTIS | 970 | ARMPTV | 1228 | WGQGTL | 1286 | CAGGTGCAGCTGGTGCAGTCTGGGGGA | 1527 | QVQLVQSGGG | 1810 | | |
| 75589 | 7 | GGLVQPGG | | MS | | PGKGL | | SEKYYV | | RDNAK | | YSGYDK | | VTVSS | | GGCTTGGTCCAGCCGGGGGGGTCCCTG | | LVQPGGSLRL | | | |
| | | SLRLSCAA | | | | EWVA | | DSLKG | | NLLYL | | | | | | AGACTCTCCTGTGCAGCCTCTAGATTC | | SCAASRFTFN | | | |
| | | SR | | | | | | | | QMNIL | | | | | | ACCTTTAATAGTTATTGGATGAGTTGG | | SYWMSWVRQA | | | |
| | | | | | | | | | | RVEDT | | | | | | GTCCGCCAGGCTCCAGGGAAGGGACTG | | PGKGLEWVAS | | | |
| | | | | | | | | | | AVYYC | | | | | | GAGTGGGTGGCCAGCATAAAGCAAGAT | | IKQDGSEKYY | | | |
| | | | | | | | | | | | | | | | | GGAAGTGAGAAATACTATGTGGACTCT | | VDSLKGRFTI | | | |
| | | | | | | | | | | | | | | | | TTGAAGGGCCGATTCACCATTTCCAGA | | SRDNAKNLLY | | | |
| | | | | | | | | | | | | | | | | GACAACGCCAAGAATTTATTGTATCTG | | LQMNILRVED | | | |
| | | | | | | | | | | | | | | | | CAAATGAACATCCTGAGAGTCGAGGAC | | TAVYYCARMP | | | |
| | | | | | | | | | | | | | | | | ACGGCTGTATATTACTGTGCGAGGATG | | TVYSGYDKWG | | | |
| | | | | | | | | | | | | | | | | CCGACGGTTTATAGTGGCTACGATAAG | | QGTLVTVSS | | | |
| | | | | | | | | | | | | | | | | TGGGGCCAGGGAACCCTGGTCACCGTC | | | | | |
| | | | | | | | | | | | | | | | | TCCTCA | | | | | |
| ADI- | IGHV5- | QVQLVQSG | 167 | YRFGNHW | 390 | WVRQA | 504 | IIYPDD | 726 | QVTIS | 971 | VRRPRV | 1229 | WGQGTT | 1287 | CAGGTGCAGCTGGTGCAGTCTGGACCA | 1528 | QVQLVQSGPE | 1811 | | |
| 75763 | 51 | PEVKKSGE | | IG | | PGKGL | | SDTRYG | | VDKSI | | QGILVA | | VTVSS | | GAGGTGAAAAAGTCCGGGGAGTCTCTG | | VKKSGESLKI | | | |
| | | SLKISCKA | | | | EWMG | | PSFEG | | STAYL | | SDPFDI | | | | AAGATCTCCTGTAAGGCTTCTGGATAT | | SCKASGYRFG | | | |
| | | SG | | | | | | | | QWSSL | | | | | | AGGTTTGGCAACCATTGGATCGGCTGG | | NHWIGWVRQA | | | |
| | | | | | | | | | | KASDT | | | | | | GTGCGCCAGGCGCCCGGGAAAGGCCTG | | PGKGLEWMGI | | | |
| | | | | | | | | | | AMYYC | | | | | | GAGTGGATGGGGATCATCTATCCTGAT | | IYPDDSDTRY | | | |
| | | | | | | | | | | | | | | | | GACTCTGATACCAGATACGGCCCGTCC | | GPSFEGQVTI | | | |
| | | | | | | | | | | | | | | | | TTCGAAGGCCAGGTCACCATCTCAGTC | | SVDKSISTAY | | | |
| | | | | | | | | | | | | | | | | GACAAGTCCATCAGCACCGCCTACTTG | | LQWSSLKASD | | | |
| | | | | | | | | | | | | | | | | CAGTGGAGCAGCCTGAAGGCCTCGGAC | | TAMYYCVRRP | | | |
| | | | | | | | | | | | | | | | | ACCGCCATGTATTACTGTGTGAGACGA | | RVQGILVASD | | | |
| | | | | | | | | | | | | | | | | CCTAGGGTTCAGGGAATTCTTGTCGCG | | PFDIWGQGTT | | | |
| | | | | | | | | | | | | | | | | AGTGATCCTTTTGATATCTGGGGCCAA | | VTVSS | | | |
| | | | | | | | | | | | | | | | | GGGACAACGGTCACCGTCTCCTCA | | | | | |
| ADI- | IGHV3- | QVQLEQSG | 19 | FTLSSHS | 391 | WVRQA | 431 | LISLGA | 727 | RFTIS | 935 | TREISG | 1230 | WGQGTL | 1286 | CAGGTCCAGCTGGAGCAGTCTGGGGGA | 1529 | QVQLEQSGGG | 1812 | | |
| 75582 | 30 | GGVVQPGR | | MH | | PGKGL | | NNKYYA | | RDNSK | | GTKGMY | | VTVSS | | GGCGTGGTCCAGCCTGGGAGGTCCCTG | | VVQPGRSLRL | | | |
| | | SLRLSCAA | | | | EWVA | | DSVKG | | NTLYL | | SGSYFL | | | | AGACTCTCCTGTGCAGCCTCTGGATTC | | SCAASGFTLS | | | |
| | | SG | | | | | | | | QMNSL | | FSGFDY | | | | ACCCTCAGTAGTCATTCTATGCACTGG | | SHSMHWVRQA | | | |
| | | | | | | | | | | RAEDT | | | | | | GTCCGCCAGGCTCCAGGCAAGGGGCTG | | PGKGLEWVAL | | | |
| | | | | | | | | | | AIYYC | | | | | | GAGTGGGTGGCACTTATATCACTTGGT | | ISLGANNKYY | | | |
| | | | | | | | | | | | | | | | | GCAAATAATAAATACTACGCAGACTCC | | ADSVKGRFTI | | | |
| | | | | | | | | | | | | | | | | GTGAAGGGCCGATTCACCATCTCCAGA | | SRDNSKNTLY | | | |
| | | | | | | | | | | | | | | | | GACAATTCCAAGAACACGCTGTATCTG | | LQMNSLRAED | | | |
| | | | | | | | | | | | | | | | | CAAATGAACAGCCTGAGAGCTGAGGAC | | TAIYYCTREI | | | |
| | | | | | | | | | | | | | | | | ACGGCTATATATTACTGTACAAGAGAA | | SGGTKGMYSG | | | |
| | | | | | | | | | | | | | | | | ATATCCGGGGGTACTAAGGGCATGTAT | | SYFLFSGFDY | | | |
| | | | | | | | | | | | | | | | | AGTGGGAGCTACTTCCTCTTTTCCGGC | | WGQGTLVTVS | | | |
| | | | | | | | | | | | | | | | | TTTGACTACTGGGGCCAGGGAACCCTG | | S | | | |
| | | | | | | | | | | | | | | | | GTCACCGTCTCCTCA | | | | | |
| ADI- | IGHV2- | QVTLKQSG | 168 | FSLSSNG | 392 | WIRQP | 505 | VIYWDE | 728 | RLTIT | 972 | AHTTRY | 1231 | WGQGTL | 1286 | CAGGTCACGCTGAAGCAGTCTGGTCCT | 1530 | QVTLKQSGPT | 1813 | | |
| 75746 | 5 | PTVVKPTQ | | EGVA | | PGKAL | | EERYSP | | KDTSK | | SNRFDP | | VTVSS | | ACCGTGGTGAAACCCACACAGACCCTC | | VVKPTQTLTL | | | |
| | | TLTLTCNF | | | | EWFA | | SLKS | | NQVVL | | | | | | ACGCTGACCTGCAACTTCTCTGGGTTC | | TCNFSGFSLS | | | |
| | | SG | | | | | | | | TMTDM | | | | | | TCACTCAGCAGTAATGGAGAGGGTGTG | | SNGEGVAWIR | | | |
| | | | | | | | | | | DPVDT | | | | | | GCCTGGATCCGTCAGCCCCCAGGAAAG | | QPPGKALEWF | | | |
| | | | | | | | | | | GTYYC | | | | | | GCCCTGGAGTGGTTTGCAGTCATTTAT | | AVIYWDEEER | | | |
| | | | | | | | | | | | | | | | | TGGGATGAAGAGGAGCGCTACAGCCCA | | YSPSLKSRLT | | | |
| | | | | | | | | | | | | | | | | TCTCTCAAGAGCAGGCTAACCATCACC | | ITKDTSKNQV | | | |
| | | | | | | | | | | | | | | | | AAGGACACCTCTAAAAACCAGGTGGTC | | VLTMTDMDPV | | | |
| | | | | | | | | | | | | | | | | CTTACAATGACCGACATGGACCCTGTG | | DTGTYYCAHT | | | |
| | | | | | | | | | | | | | | | | GACACAGGCACATATTACTGTGCACAC | | TRYSNRFDPW | | | |
| | | | | | | | | | | | | | | | | ACTACTCGATATAGCAACAGGTTCGAC | | GQGTLVTVSS | | | |
| | | | | | | | | | | | | | | | | CCCTGGGGCCAGGGAACCCTGGTCACC | | | | | |
| | | | | | | | | | | | | | | | | GTCTCCTCA | | | | | |
| ADI- | IGHV2- | PGHIEGVW | 169 | FSLSTSG | 393 | WIRQP | 506 | RIDWDG | 729 | RLTIS | 973 | ARCQVG | 1232 | WGQGTL | 1286 | CCAGGTCACATTGAAGGAGTCTGGTCT | 1531 | PGHIEGVWSA | 1814 | | |
| 75726 | 70 | SALVKPTE | | MSVS | | PGGAL | | YKYYST | | KDTSK | | MENN | | VTVSS | | GCGCTGGTGAAGCCCACAGAGACCCTC | | LVKPTETLTL | | | |
| | | TLTLTCTL | | | | EWLA | | TLET | | NQVVL | | | | | | ACACTGACCTGCACCTTGTATGGGTTC | | TCTLYGFSLS | | | |
| | | YG | | | | | | | | SLTNM | | | | | | TCACTCAGCACTAGTGGAATGTCTGTG | | TSGMSVSWIR | | | |
| | | | | | | | | | | DPVDT | | | | | | AGCTGGATCCGTCAGCCCCCCGGGGGG | | QPPGGALEWL | | | |
| | | | | | | | | | | ATYYC | | | | | | GCCCTGGAGTGGCTTGCACGCATTGAT | | ARIDWDGYKY | | | |
| | | | | | | | | | | | | | | | | TGGGATGGTTATAAATATTACAGCACA | | YSTTLETRLT | | | |
| | | | | | | | | | | | | | | | | ACTCTGGAGACCAGGCTCACCATCTCC | | ISKDTSKNQV | | | |
| | | | | | | | | | | | | | | | | AAGGACACCTCCAAAAACCAGGTGGTC | | VLSLTNMDPV | | | |
| | | | | | | | | | | | | | | | | CTTTCACTGACCAACATGGACCCTGTG | | DTATYYCARC | | | |
| | | | | | | | | | | | | | | | | GACACAGCCACGTATTACTGTGCACGG | | QVGMENNWGQ | | | |
| | | | | | | | | | | | | | | | | TGCCAGGTGGGGATGGAGAACAACTGG | | GTLVTVSS | | | |
| | | | | | | | | | | | | | | | | GGCCAGGGAACCCTGGTCACCGTCTCC | | | | | |
| | | | | | | | | | | | | | | | | TCA | | | | | |
| ADI- | IGHV1- | QVQLLQSG | 170 | YTFTSYY | 299 | WVRQA | 507 | IVNPGG | 730 | RVSMT | 974 | ARTRIA | 1233 | WGQGTL | 1286 | CAGGTGCAGCTGCTGCAGTCTGGGGCT | 1532 | QVQLLQSGAE | 1815 | | |
| 75851 | 46 | AEVKEPGA | | MH | | PAQGL | | GDTSYA | | RDTSA | | AATDYF | | VTVSS | | GAGGTGAAGGAGCCTGGGGCCTCAGTG | | VKEPGASVKV | | | |
| | | SVKVSCKA | | | | EWMG | | QKFQG | | STVYL | | DY | | | | AAGGTTTCCTGCAAGGCATCTGGGTAC | | SCKASGYTFT | | | |
| | | SG | | | | | | | | ELSSL | | | | | | ACCTTCACCAGCTACTATATGCACTGG | | SYYMHWVRQA | | | |
| | | | | | | | | | | RSEDT | | | | | | GTGCGACAGGCCCCTGCACAAGGGCTT | | PAQGLEWMGI | | | |
| | | | | | | | | | | AIYYC | | | | | | GAGTGGATGGGAATAGTCAACCCTGGT | | VNPGGGDTSY | | | |
| | | | | | | | | | | | | | | | | GGTGGTGACACAAGCTACGCACAGAAG | | AQKFQGRVSM | | | |
| | | | | | | | | | | | | | | | | TTCCAGGGCAGAGTCTCCATGACCAGG | | TRDTSASTVY | | | |
| | | | | | | | | | | | | | | | | GACACGTCCGCGAGCACAGTCTACTTG | | LELSSLRSED | | | |
| | | | | | | | | | | | | | | | | GAGCTGAGCAGCCTGAGATCTGAGGAC | | TAIYYCARTR | | | |
| | | | | | | | | | | | | | | | | ACGGCCATTTATTACTGTGCGAGAACC | | IAAATDYFDY | | | |
| | | | | | | | | | | | | | | | | CGCATAGCAGCAGCCACGGACTACTTT | | WGQGTLVTVS | | | |
| | | | | | | | | | | | | | | | | GACTACTGGGGCCAGGGAACCCTGGTC | | S | | | |
| | | | | | | | | | | | | | | | | ACCGTCTCCTCA | | | | | |
| ADI- | IGHV1- | QVQLVQSG | 50 | GTFSSSA | 394 | WVRQA | 433 | GIIPIF | 731 | RVTIT | 975 | ATDHVT | 1234 | WGQGTT | 1287 | CAGGTGCAGCTGGTGCAGTCTGGGGCT | 1533 | QVQLVQSGAE | 1816 | | |
| 75853 | 69 | AEVKKPGS | | IN | | PGQGL | | GTTNYA | | ADELT | | SPLYGL | | VTVSS | | GAGGTGAAGAAGCCTGGGTCCTCGGTG | | VKKPGSSVKV | | | |
| | | SVKVSCKA | | | | EWMG | | QKFQG | | NTAYM | | DV | | | | AAGGTCTCCTGCAAGGCTTCTGGAGGC | | SCKASGGTFS | | | |
| | | SG | | | | | | | | ELSSL | | | | | | ACCTTCAGCAGTAGTGCTATCAACTGG | | SSAINWVRQA | | | |
| | | | | | | | | | | RSEDT | | | | | | GTGCGACAGGCCCCTGGACAAGGGCTT | | PGQGLEWMGG | | | |
| | | | | | | | | | | AVYYC | | | | | | GAGTGGATGGGAGGAATCATCCCTATT | | IIPIFGTTNY | | | |
| | | | | | | | | | | | | | | | | TTTGGTACAACAAACTACGCACAGAAG | | AQKFQGRVTI | | | |
| | | | | | | | | | | | | | | | | TTCCAGGGCAGAGTCACGATTACCGCG | | TADELTNTAY | | | |
| | | | | | | | | | | | | | | | | GACGAATTAACGAACACAGCCTACATG | | MELSSLRSED | | | |
| | | | | | | | | | | | | | | | | GAGCTGAGCAGCCTGAGATCTGAGGAC | | TAVYYCATDH | | | |
| | | | | | | | | | | | | | | | | ACGGCCGTGTATTACTGTGCGACAGAT | | VTSPLYGLDV | | | |
| | | | | | | | | | | | | | | | | CACGTTACTTCGCCCCTCTACGGTTTG | | WGQGTTVTVS | | | |
| | | | | | | | | | | | | | | | | GACGTCTGGGGCCAAGGGACCACGGTC | | S | | | |
| | | | | | | | | | | | | | | | | ACCGTCTCCTCA | | | | | |
| ADI- | IGHV3- | QVQLVQSG | 17 | FTFDDYA | 214 | WVRQA | 437 | GISGNS | 732 | RFTIS | 976 | AKTLAP | 1235 | WGQGTT | 1287 | CAGGTGCAGCTGGTGCAGTCTGGGGGA | 1534 | QVQLVQSGGG | 1817 | | |
| 75863 | 9 | GGLVQPGR | | MH | | PGKGL | | GTIGYA | | RDNAK | | DRAHYD | | VTVSS | | GGGTTGGTACAGCCTGGCAGGTCCCTG | | LVQPGRSLRL | | | |
| | | SLRLSCAA | | | | EWVS | | DSVKG | | NSLYL | | TANDAF | | | | AGACTCTCCTGTGCAGCCTCTGGATTC | | SCAASGFTFD | | | |
| | | SG | | | | | | | | QMNRL | | DI | | | | ACCTTTGATGATTATGCCATGCACTGG | | DYAMHWVRQA | | | |
| | | | | | | | | | | RAEDT | | | | | | GTCCGGCAAGCTCCAGGGAAGGGCCTG | | PGKGLEWVSG | | | |
| | | | | | | | | | | ALYYC | | | | | | GAGTGGGTCTCAGGTATTAGTGGGAAT | | ISGNSGTIGY | | | |
| | | | | | | | | | | | | | | | | AGTGGTACCATAGGCTATGCGGACTCT | | ADSVKGRFTI | | | |
| | | | | | | | | | | | | | | | | GTGAAGGGCCGATTCACCATCTCCAGA | | SRDNAKNSLY | | | |
| | | | | | | | | | | | | | | | | GACAACGCCAAGAACTCCCTCTATCTG | | LQMNRLRAED | | | |
| | | | | | | | | | | | | | | | | CAAATGAACAGACTGAGAGCTGAGGAC | | TALYYCAKTL | | | |
| | | | | | | | | | | | | | | | | ACGGCCTTGTATTACTGTGCAAAAACT | | APDRAHYDTA | | | |
| | | | | | | | | | | | | | | | | TTGGCCCCAGACAGGGCCCATTACGAT | | NDAFDIWGQG | | | |
| | | | | | | | | | | | | | | | | ACCGCGAACGATGCTTTTGATATCTGG | | TTVTVSS | | | |
| | | | | | | | | | | | | | | | | GGCCAAGGGACCACGGTCACCGTCTCC | | | | | |
| | | | | | | | | | | | | | | | | TCA | | | | | |
| ADI- | IGHV3- | QVQLVESG | 171 | FIFDDYA | 395 | WVRQA | 437 | GINWNS | 733 | RFTIS | 977 | AKYRVA | 1236 | WGQGTT | 1287 | CAGGTCCAGCTGGTGGAGTCTGGGGGA | 1535 | QVQLVESGGG | 1818 | | |
| 75753 | 9 | GGLVQPGR | | MH | | PGKGL | | GTIAYA | | RDNAK | | ELTNDV | | VTVSS | | GGCTTGGTACAGCCTGGCAGGTCACTG | | LVQPGRSLRV | | | |
| | | SLRVSCAA | | | | EWVS | | DSVKG | | NSLYL | | FDV | | | | AGAGTCTCCTGTGCAGCCTCTGGATTC | | SCAASGFIFD | | | |
| | | SG | | | | | | | | EMHSL | | | | | | ATCTTTGATGATTATGCCATGCACTGG | | DYAMHWVRQA | | | |
| | | | | | | | | | | KPDDT | | | | | | GTCCGGCAAGCTCCAGGGAAGGGCCTG | | PGKGLEWVSG | | | |
| | | | | | | | | | | ALYYC | | | | | | GAGTGGGTCTCAGGAATTAATTGGAAT | | INWNSGTIAY | | | |
| | | | | | | | | | | | | | | | | AGTGGTACCATAGCCTATGCGGACTCT | | ADSVKGRFTI | | | |
| | | | | | | | | | | | | | | | | GTGAAGGGCCGGTTCACCATCTCCAGA | | SRDNAKNSLY | | | |
| | | | | | | | | | | | | | | | | GACAACGCCAAGAACTCCCTGTATCTG | | LEMHSLKPDD | | | |
| | | | | | | | | | | | | | | | | GAAATGCACAGTCTGAAACCTGACGAC | | TALYYCAKYR | | | |
| | | | | | | | | | | | | | | | | ACGGCCTTATATTACTGTGCAAAATAC | | VAELTNDVFD | | | |
| | | | | | | | | | | | | | | | | AGGGTGGCGGAATTAACGAATGATGTT | | VWGQGTTVTV | | | |
| | | | | | | | | | | | | | | | | TTTGATGTCTGGGGCCAAGGGACCACG | | SS | | | |
| | | | | | | | | | | | | | | | | GTCACCGTCTCCTCA | | | | | |
| ADI- | IGHV3- | QVQLVQSG | 10 | FTFSNYA | 396 | WVRQA | 431 | VIWYDG | 734 | RFTIS | 779 | ARDENG | 1237 | WGQGTL | 1286 | CAGGTGCAGCTGGTGCAGTCTGGGGGG | 1536 | QVQLVQSGGG | 1819 | | |
| 75756 | 33 | GGVVQPGR | | MH | | PGKGL | | SFKFYA | | RDNSK | | HEVMVT | | VTVSS | | GGCGTGGTCCAGCCTGGGAGGTCCCTG | | VVQPGRSLRL | | | |
| | | SLRLSCAA | | | | EWVA | | DSVKG | | NTVYL | | HFDY | | | | AGACTCTCCTGTGCAGCGTCTGGATTC | | SCAASGFTFS | | | |
| | | SG | | | | | | | | QMNSL | | | | | | ACCTTCAGTAACTATGCCATGCACTGG | | NYAMHWVRQA | | | |
| | | | | | | | | | | RAEDT | | | | | | GTCCGCCAGGCTCCAGGCAAGGGGCTG | | PGKGLEWVAV | | | |
| | | | | | | | | | | AVYYC | | | | | | GAATGGGTGGCAGTTATTTGGTATGAT | | IWYDGSFKFY | | | |
| | | | | | | | | | | | | | | | | GGCAGTTTTAAATTCTATGCAGACTCC | | ADSVKGRFTI | | | |
| | | | | | | | | | | | | | | | | GTGAAGGGCCGATTCACCATCTCCAGA | | SRDNSKNTVY | | | |
| | | | | | | | | | | | | | | | | GACAATTCCAAGAACACGGTGTATCTG | | LQMNSLRAED | | | |
| | | | | | | | | | | | | | | | | CAAATGAACAGCCTGAGAGCCGAGGAC | | TAVYYCARDE | | | |
| | | | | | | | | | | | | | | | | ACGGCTGTGTATTACTGTGCGAGAGAT | | NGHEVMVTHF | | | |
| | | | | | | | | | | | | | | | | GAGAACGGACATGAAGTCATGGTTACT | | DYWGQGTLVT | | | |
| | | | | | | | | | | | | | | | | CACTTTGACTACTGGGGCCAGGGAACC | | VSS | | | |
| | | | | | | | | | | | | | | | | CTGGTCACCGTCTCCTCA | | | | | |
| ADI- | IGHV1- | QVPLKQSG | 172 | YTFTSYY | 397 | WVRQA | 433 | MINPTG | 735 | RVTMS | 978 | ARSQVV | 1238 | WGQGTL | 1286 | CAGGTGCCGCTGAAGCAGTCTGGGGCT | 1537 | QVPLKQSGAE | 1820 | | |
| 75769 | 46 | AEVKKPGA | | VH | | PGQGL | | GSTGYA | | RDTST | | PATSHL | | VTVSS | | GAGGTGAAGAAGCCTGGGGCCTCAGTG | | VKKPGASVKI | | | |
| | | SVKISCKA | | | | EWMG | | QKFQG | | STVYM | | DY | | | | AAGATTTCCTGCAAGGCATCTGGATAC | | SCKASGYTFT | | | |
| | | SG | | | | | | | | ELTSL | | | | | | ACCTTCACCAGCTACTATGTACACTGG | | SYYVHWVRQA | | | |
| | | | | | | | | | | RSEDT | | | | | | GTGCGACAGGCCCCTGGACAAGGGCTT | | PGQGLEWMGM | | | |
| | | | | | | | | | | AVYYC | | | | | | GAGTGGATGGGAATGATCAACCCTACT | | INPTGGSTGY | | | |
| | | | | | | | | | | | | | | | | GGTGGTAGCACAGGTTACGCACAGAAG | | AQKFQGRVTM | | | |
| | | | | | | | | | | | | | | | | TTCCAGGGCAGAGTCACCATGTCCAGG | | SRDTSTSTVY | | | |
| | | | | | | | | | | | | | | | | GACACGTCCACGAGCACAGTCTACATG | | MELTSLRSED | | | |
| | | | | | | | | | | | | | | | | GAACTGACCAGCCTGAGATCTGAGGAC | | TAVYYCARSQ | | | |
| | | | | | | | | | | | | | | | | ACGGCCGTTTATTACTGTGCGAGAAGC | | VVPATSHLDY | | | |
| | | | | | | | | | | | | | | | | CAAGTAGTGCCAGCTACTAGTCACCTT | | WGQGTLVTVS | | | |
| | | | | | | | | | | | | | | | | GACTACTGGGGCCAGGGAACCCTGGTC | | S | | | |
| | | | | | | | | | | | | | | | | ACCGTCTCCTCA | | | | | |
| ADI- | IGHV3- | QVQLVQSG | 10 | FTFSSYG | 262 | WVRQA | 431 | VIWYDG | 679 | RFTIS | 777 | ARAGYS | 1239 | WGKGTT | 1291 | CAGGTCCAGCTGGTGCAGTCTGGGGGA | 1538 | QVQLVQSGGG | 1821 | | |
| 75772 | 33 | GGVVQPGR | | MH | | PGKGL | | SNKYYA | | RDNSK | | SYYYYM | | VTVSS | | GGCGTGGTCCAGCCTGGGAGGTCCCTG | | VVQPGRSLRL | | | |
| | | SLRLSCAA | | | | EWVA | | DSVKG | | NTLYL | | DV | | | | AGACTCTCCTGTGCAGCGTCTGGATTC | | SCAASGFTFS | | | |
| | | SG | | | | | | | | QMNSL | | | | | | ACCTTCAGTAGCTATGGCATGCACTGG | | SYGMHWVRQA | | | |
| | | | | | | | | | | RAEDT | | | | | | GTCCGCCAGGCTCCAGGCAAGGGGCTG | | PGKGLEWVAV | | | |
| | | | | | | | | | | AVYYC | | | | | | GAGTGGGTGGCAGTTATATGGTATGAT | | IWYDGSNKYY | | | |
| | | | | | | | | | | | | | | | | GGAAGTAATAAATACTATGCAGACTCC | | ADSVKGRFTI | | | |
| | | | | | | | | | | | | | | | | GTGAAGGGCCGATTCACCATCTCCAGA | | SRDNSKNTLY | | | |
| | | | | | | | | | | | | | | | | GACAATTCCAAGAACACGCTGTATCTG | | LQMNSLRAED | | | |
| | | | | | | | | | | | | | | | | CAAATGAACAGCCTGAGAGCCGAGGAC | | TAVYYCARAG | | | |
| | | | | | | | | | | | | | | | | ACGGCTGTGTATTACTGTGCGAGAGCG | | YSSYYYYMDV | | | |
| | | | | | | | | | | | | | | | | GGGTACAGTAGCTACTACTACTACATG | | WGKGTTVTVS | | | |
| | | | | | | | | | | | | | | | | GACGTCTGGGGCAAAGGGACCACGGTC | | S | | | |
| | | | | | | | | | | | | | | | | ACCGTCTCCTCA | | | | | |
| ADI- | IGHV1- | QVQLQESG | 173 | GTFSTYH | 398 | WVRQA | 508 | GIIPLF | 736 | RVTIT | 979 | ASEDCS | 1240 | WGQGTL | 1286 | CAGGTGCAGCTGCAGGAGTCCGGGACT | 1539 | QVQLQESGTE | 1822 | | |
| 75777 | 69 | TEVKKPGS | | VS | | PGRGL | | GSANYT | | ADKST | | GGSCYF | | VTVSS | | GAGGTGAAGAAGCCTGGGTCCTCGGTG | | VKKPGSSVKV | | | |
| | | SVKVSCKA | | | | EWMG | | QRFQG | | RTVYM | | GVGSPY | | | | AAGGTCTCCTGCAAGGCCTCTGGAGGC | | SCKASGGTFS | | | |
| | | SG | | | | | | | | ELRSL | | ARGFET | | | | ACCTTCAGCACCTATCATGTCAGCTGG | | TYHVSWVRQA | | | |
| | | | | | | | | | | RSDDT | | | | | | GTGCGACAGGCCCCTGGACGAGGCCTT | | PGRGLEWMGG | | | |
| | | | | | | | | | | AVYYC | | | | | | GAGTGGATGGGAGGGATCATCCCTCTC | | IIPLFGSANY | | | |
| | | | | | | | | | | | | | | | | TTTGGTTCAGCAAACTACACACAGAGG | | TQRFQGRVTI | | | |
| | | | | | | | | | | | | | | | | TTCCAGGGCAGAGTCACGATTACCGCG | | TADKSTRTVY | | | |
| | | | | | | | | | | | | | | | | GACAAATCCACGCGCACAGTCTACATG | | MELRSLRSDD | | | |
| | | | | | | | | | | | | | | | | GAGTTGAGGAGTCTGAGATCTGACGAC | | TAVYYCASED | | | |
| | | | | | | | | | | | | | | | | ACGGCCGTATATTATTGTGCGAGCGAG | | CSGGSCYFGV | | | |
| | | | | | | | | | | | | | | | | GATTGTAGTGGTGGTAGCTGCTACTTC | | GSPYARGFET | | | |
| | | | | | | | | | | | | | | | | GGGGTTGGGAGTCCTTATGCACGAGGA | | WGQGTLVTVS | | | |
| | | | | | | | | | | | | | | | | TTCGAAACCTGGGGCCAGGGAACCCTG | | S | | | |
| | | | | | | | | | | | | | | | | GTCACCGTCTCCTCA | | | | | |
| ADI- | IGHV5- | QVQLVQSG | 174 | YSFTSYW | 399 | WVRQM | 476 | IIYPGD | 635 | QVTFS | 980 | ARRDSS | 1241 | WGKGTT | 1291 | CAGGTCCAGCTGGTGCAGTCTGGAGCA | 1540 | QVQLVQSGAE | 1823 | | |
| 75781 | 51 | AEVKKPGE | | IA | | PGKGL | | SDTRYS | | ADKSI | | WNDMDV | | VTVSS | | GAGGTGAAAAAGCCCGGGGAGTCTCTG | | VKKPGESLKI | | | |
| | | SLKISCKG | | | | EWMG | | PSFQG | | STAYL | | | | | | AAGATCTCCTGTAAGGGATCTGGATAC | | SCKGSGYSFT | | | |
| | | SG | | | | | | | | QWTSL | | | | | | AGCTTTACCAGCTACTGGATCGCCTGG | | SYWIAWVRQM | | | |
| | | | | | | | | | | KASDT | | | | | | GTGCGCCAGATGCCCGGGAAAGGCCTG | | PGKGLEWMGI | | | |
| | | | | | | | | | | AIYYC | | | | | | GAGTGGATGGGGATCATCTATCCTGGT | | IYPGDSDTRY | | | |
| | | | | | | | | | | | | | | | | GACTCTGATACCAGATACAGCCCGTCT | | SPSFQGQVTF | | | |
| | | | | | | | | | | | | | | | | TTCCAAGGCCAGGTCACCTTCTCAGCC | | SADKSISTAY | | | |
| | | | | | | | | | | | | | | | | GACAAGTCCATCAGCACCGCCTACCTG | | LQWTSLKASD | | | |
| | | | | | | | | | | | | | | | | CAGTGGACCAGCCTGAAGGCCTCGGAC | | TAIYYCARRD | | | |
| | | | | | | | | | | | | | | | | ACCGCCATTTATTACTGTGCGAGACGT | | SSWNDMDVWG | | | |
| | | | | | | | | | | | | | | | | GACAGCAGCTGGAACGACATGGACGTC | | KGTTVTVSS | | | |
| | | | | | | | | | | | | | | | | TGGGGCAAAGGGACCACGGTCACCGTC | | | | | |
| | | | | | | | | | | | | | | | | TCCTCA | | | | | |
| ADI- | IGHV3- | QVQLVESG | 175 | FTFEDFA | 400 | WVRQA | 437 | GISWNG | 737 | RFILS | 981 | AKDEGS | 1242 | WGQGTL | 1286 | CAGGTGCAGCTGGTGGAGTCTGGGGGA | 1541 | QVQLVESGGG | 1824 | | |
| 75752 | 9 | GGLVQPGR | | LH | | PGKGL | | GVIGYA | | RDNDK | | EGDQQP | | VTVSS | | GGCTTGGTACAGCCTGGCAGGTCCCTG | | LVQPGRSLTL | | | |
| | | SLTLSCAA | | | | EWVS | | DSVKG | | NSLYL | | LDY | | | | ACACTCTCCTGTGCAGCCTCTGGATTC | | SCAASGFTFE | | | |
| | | SG | | | | | | | | HMRGL | | | | | | ACCTTTGAAGATTTTGCCTTGCACTGG | | DFALHWVRQA | | | |
| | | | | | | | | | | RAEDT | | | | | | GTCCGGCAAGCTCCAGGGAAGGGCCTG | | PGKGLEWVSG | | | |
| | | | | | | | | | | ALYYC | | | | | | GAGTGGGTCTCAGGTATTAGTTGGAAT | | ISWNGGVIGY | | | |
| | | | | | | | | | | | | | | | | GGTGGTGTCATAGGCTATGCGGACTCT | | ADSVKGRFIL | | | |
| | | | | | | | | | | | | | | | | GTGAAGGGCCGATTCATCCTCTCCAGA | | SRDNDKNSLY | | | |
| | | | | | | | | | | | | | | | | GACAACGACAAGAATTCCCTGTATCTA | | LHMRGLRAED | | | |
| | | | | | | | | | | | | | | | | CATATGCGGGGTCTGAGAGCTGAGGAC | | TALYYCAKDE | | | |
| | | | | | | | | | | | | | | | | ACGGCCTTGTATTACTGTGCAAAAGAT | | GSEGDQQPLD | | | |
| | | | | | | | | | | | | | | | | GAAGGATCTGAGGGGGACCAGCAGCCC | | YWGQGTLVTV | | | |
| | | | | | | | | | | | | | | | | CTTGACTACTGGGGCCAGGGAACCCTG | | SS | | | |
| | | | | | | | | | | | | | | | | GTCACCGTCTCCTCA | | | | | |
| ADI- | IGHV3- | QVQLQQSG | 110 | FTFSNFW | 401 | WVRQA | 486 | RINSDG | 738 | RFTIS | 821 | ARDLES | 1169 | WGQGTL | 1286 | CAGGTGCAGCTGCAGCAGTCTGGGGGA | 1542 | QVQLQQSGGG | 1825 | | |
| 75676 | 74 | GGLVQPGG | | MH | | PGKGL | | GSTSYA | | RDNAK | | GSFHPG | | VTVSS | | GGCTTAGTTCAGCCTGGGGGGTCCCTG | | LVQPGGSLRL | | | |
| | | SLRLSCAA | | | | VWVS | | DSVKG | | NTLYL | | LVRVP | | | | AGACTCTCCTGTGCAGCCTCTGGATTC | | SCAASGFTFS | | | |
| | | SG | | | | | | | | QMNSL | | | | | | ACCTTCAGTAACTTCTGGATGCACTGG | | NFWMHWVRQA | | | |
| | | | | | | | | | | RAEDT | | | | | | GTCCGCCAAGCTCCAGGGAAGGGGCTG | | PGKGLVWVSR | | | |
| | | | | | | | | | | AVYYC | | | | | | GTGTGGGTCTCACGTATTAATAGTGAT | | INSDGGSTSY | | | |
| | | | | | | | | | | | | | | | | GGGGGTAGCACAAGCTACGCGGACTCC | | ADSVKGRFTI | | | |
| | | | | | | | | | | | | | | | | GTGAAGGGCCGATTCACCATCTCCAGA | | SRDNAKNTLY | | | |
| | | | | | | | | | | | | | | | | GACAACGCCAAGAACACGCTGTATCTG | | LQMNSLRAED | | | |
| | | | | | | | | | | | | | | | | CAAATGAACAGTCTGAGAGCCGAGGAC | | TAVYYCARDL | | | |
| | | | | | | | | | | | | | | | | ACGGCTGTATATTACTGTGCAAGAGAC | | ESGSFHPGLV | | | |
| | | | | | | | | | | | | | | | | CTGGAAAGTGGGAGCTTTCATCCCGGC | | RVPWGQGTLV | | | |
| | | | | | | | | | | | | | | | | CTGGTTCGGGTCCCCTGGGGCCAGGGA | | TVSS | | | |
| | | | | | | | | | | | | | | | | ACCCTGGTCACCGTCTCTTCA | | | | | |
| ADI- | IGHV- | QVQLVQSG | 50 | GTFSSFP | 402 | WVRQA | 433 | RIIPIL | 739 | RVTIT | 982 | ARDVGY | 1243 | WGQGTL | 1286 | CAGGTGCAGCTGGTGCAGTCTGGGGCT | 1543 | QVQLVQSGAE | 1826 | | |
| 75731 | 69 | AEVKKPGS | | IS | | PGQGL | | GVADYA | | ADKST | | SGSGSS | | VTVSS | | GAGGTGAAGAAGCCTGGGTCCTCGGTG | | VKKPGSSVKV | | | |
| | | SVKVSCKA | | | | EWMG | | QKFQG | | GTAYM | | YYFDS | | | | AAGGTCTCCTGCAAGGCTTCTGGAGGC | | SCKASGGTFS | | | |
| | | SG | | | | | | | | ELSSL | | | | | | ACCTTCAGCAGCTTTCCTATTAGTTGG | | SFPISWVRQA | | | |
| | | | | | | | | | | RSEDT | | | | | | GTGCGACAGGCCCCTGGACAAGGGCTT | | PGQGLEWMGR | | | |
| | | | | | | | | | | AVYYC | | | | | | GAGTGGATGGGAAGGATCATCCCTATC | | IIPILGVADY | | | |
| | | | | | | | | | | | | | | | | CTTGGTGTAGCAGACTACGCACAAAAG | | AQKFQGRVTI | | | |
| | | | | | | | | | | | | | | | | TTCCAGGGCAGAGTCACGATTACCGCG | | TADKSTGTAY | | | |
| | | | | | | | | | | | | | | | | GACAAATCCACGGGCACCGCGTATATG | | MELSSLRSED | | | |
| | | | | | | | | | | | | | | | | GAGTTGAGCAGCCTGAGATCTGAGGAC | | TAVYYCARDV | | | |
| | | | | | | | | | | | | | | | | ACGGCCGTATATTACTGTGCGAGAGAT | | GYSGSGSSYY | | | |
| | | | | | | | | | | | | | | | | GTGGGTTACTCTGGCTCGGGGAGTTCT | | FDSWGQGTLV | | | |
| | | | | | | | | | | | | | | | | TACTACTTTGACTCCTGGGGCCAGGGA | | TVSS | | | |
| | | | | | | | | | | | | | | | | ACCCTGGTCACCGTCTCCTCA | | | | | |
| ADI- | IGHV1- | QVQLQQSG | 86 | VPLSNYA | 403 | WVRQA | 509 | GITPFS | 740 | RVTIT | 983 | ALLNRG | 1244 | WGQGTT | 1287 | CAGGTGCAGCTGCAGCAGTCTGGGGCT | 1544 | QVQLQQSGAE | 1827 | | |
| 75837 | 69 | AEVKKPGS | | IS | | PGQGL | | GTAKHA | | ADEST | | YRSSWF | | VTVSS | | GAGGTGAAGAAGCCTGGGTCCTCGGTG | | VKKPGSSVKV | | | |
| | | SVKVSCKA | | | | DWLG | | QKFQD | | NTVFM | | ERGGYY | | | | AAGGTCTCCTGCAAGGCTTCTGGAGTC | | SCKASGVPLS | | | |
| | | SG | | | | | | | | ELSSL | | YYGMDV | | | | CCCCTCAGCAACTATGCTATCAGCTGG | | NYAISWVRQA | | | |
| | | | | | | | | | | RSEDS | | | | | | GTGCGGCAGGCCCCTGGACAAGGGCTT | | PGQGLDWLGG | | | |
| | | | | | | | | | | AVYYC | | | | | | GACTGGTTGGGAGGAATTACTCCGTTT | | ITPFSGTAKH | | | |
| | | | | | | | | | | | | | | | | TCTGGCACAGCCAAGCACGCACAGAAG | | AQKFQDRVTI | | | |
| | | | | | | | | | | | | | | | | TTCCAGGACAGAGTCACGATTACCGCG | | TADESTNTVF | | | |
| | | | | | | | | | | | | | | | | GACGAATCCACGAACACAGTCTTCATG | | MELSSLRSED | | | |
| | | | | | | | | | | | | | | | | GAGTTGAGCAGCCTGAGATCTGAGGAC | | SAVYYCALLN | | | |
| | | | | | | | | | | | | | | | | TCGGCCGTCTATTACTGTGCCCTCCTA | | RGYRSSWFER | | | |
| | | | | | | | | | | | | | | | | AATCGGGGATATCGCAGCAGCTGGTTT | | GGYYYYGMDV | | | |
| | | | | | | | | | | | | | | | | GAAAGGGGGGGTTACTATTACTACGGC | | WGQGTTVTVS | | | |
| | | | | | | | | | | | | | | | | ATGGACGTCTGGGGCCAAGGGACCACG | | S | | | |
| | | | | | | | | | | | | | | | | GTCACCGTCTCCTCA | | | | | |
| ADI- | IGHV3- | QVHAGAVW | 176 | FIFSNYA | 404 | WVRQA | 431 | VISFDG | 741 | RFTIS | 984 | AKRGLG | 1245 | WGQGTL | 1286 | CAGGTACACGCTGGTGCAGTCTGGGGG | 1545 | QVHAGAVWGR | 1828 | | |
| 75728 | 30 | GRRGPAWE | | MH | | PGKGL | | DNIYYA | | RDNSK | | YCSAGT | | VTVSS | | AGGCGTGGTCCAGCCTGGGAGGTCCTG | | RGPAWEVLRL | | | |
| | | VLRLSCEG | | | | EWVA | | DSVKG | | KTMYL | | CFGEGE | | | | CGACTCTCCTGTGAAGGGTCTGGATTC | | SCEGSGFIFS | | | |
| | | SG | | | | | | | | QMNSL | | GGEFDY | | | | ATCTTCAGCAATTATGCCATGCACTGG | | NYAMHWVRQA | | | |
| | | | | | | | | | | RADDT | | | | | | GTCCGCCAGGCTCCAGGCAAGGGGCTG | | PGKGLEWVAV | | | |
| | | | | | | | | | | AVYYC | | | | | | GAGTGGGTGGCTGTCATATCATTTGAT | | ISFDGDNIYY | | | |
| | | | | | | | | | | | | | | | | GGAGATAATATATACTATGCAGACTCC | | ADSVKGRFTI | | | |
| | | | | | | | | | | | | | | | | GTGAAGGGCCGATTCACCATCTCCAGA | | SRDNSKKTMY | | | |
| | | | | | | | | | | | | | | | | GACAATTCCAAGAAGACGATGTATCTG | | LQMNSLRADD | | | |
| | | | | | | | | | | | | | | | | CAAATGAACAGCCTGAGAGCTGATGAC | | TAVYYCAKRG | | | |
| | | | | | | | | | | | | | | | | ACGGCTGTGTATTACTGTGCGAAACGG | | LGYCSAGTCF | | | |
| | | | | | | | | | | | | | | | | GGACTAGGATATTGCAGTGCTGGTACG | | GEGEGGEFDY | | | |
| | | | | | | | | | | | | | | | | TGCTTCGGCGAGGGTGAGGGAGGCGAA | | WGQGTLVTVS | | | |
| | | | | | | | | | | | | | | | | TTCGACTACTGGGGCCAGGGAACCCTG | | S | | | |
| | | | | | | | | | | | | | | | | GTCACCGTCTCCTCA | | | | | |
| ADI- | IGHV4- | QVQLQQWG | 34 | GSFGVTF | 405 | WIRQT | 510 | EISQSG | 742 | RLSIS | 985 | ARGRGS | 1246 | WGQGAL | 1285 | CAGGTACAGCTGCAGCAGTGGGGCGCG | 1546 | QVQLQQWGAG | 1829 | | |
| 75601 | 34 | AGLLKPSE | | SGGSFTG | | PGKGL | | TTNSNP | | IDTSK | | GSGSDS | | VTVSS | | GGACTATTGAAGCCCTCGGAGACCCTG | | LLKPSETLSL | | | |
| | | TLSLTCAV | | HYWN | | EWLG | | SLKS | | SHFSL | | FDS | | | | TCCCTCACCTGCGCTGTCTCTGGTGGC | | TCAVSGGSFG | | | |
| | | SG | | | | | | | | KLRSV | | | | | | TCCTTCGGTGTGACTTTCTCTGGTGGG | | VTFSGGSFTG | | | |
| | | | | | | | | | | NAADT | | | | | | TCCTTCACTGGTCATTACTGGAACTGG | | HYWNWIRQTP | | | |
| | | | | | | | | | | AIYYC | | | | | | ATCCGCCAGACCCCAGGGAAGGGGCTG | | GKGLEWLGEI | | | |
| | | | | | | | | | | | | | | | | GAGTGGCTTGGAGAAATCAGTCAGAGT | | SQSGTTNSNP | | | |
| | | | | | | | | | | | | | | | | GGTACAACCAATTCCAACCCGTCCCTC | | SLKSRLSISI | | | |
| | | | | | | | | | | | | | | | | AAGAGTCGGCTCTCCATATCAATAGAC | | DTSKSHFSLK | | | |
| | | | | | | | | | | | | | | | | ACGTCCAAGAGCCATTTCTCCCTGAAG | | LRSVNAADTA | | | |
| | | | | | | | | | | | | | | | | CTGAGGTCTGTGAACGCCGCGGACACG | | IYYCARGRGS | | | |
| | | | | | | | | | | | | | | | | GCTATCTATTACTGTGCGAGAGGCCGC | | GSGSDSFDSW | | | |
| | | | | | | | | | | | | | | | | GGTTCCGGTTCAGGGAGTGACTCTTTT | | GQGALVTVSS | | | |
| | | | | | | | | | | | | | | | | GACTCCTGGGGCCAGGGGGCCCTGGTC | | | | | |
| | | | | | | | | | | | | | | | | ACCGTCTCCTCA | | | | | |
| ADI- | IGHV1- | QVQLVQSG | 35 | YTFTNYY | 406 | WVRQV | 511 | LINPSG | 743 | RVTMT | 986 | ARDPGV | 1247 | WGQGTL | 1286 | CAGGTCCAGCTGGTGCAGTCTGGGGCT | 1547 | QVQLVQSGAE | 1830 | | |
| 75870 | 46 | AEVKKPGA | | ML | | PGQGL | | GSTSYA | | RDTST | | VDPQYP | | VTVSS | | GAGGTGAAGAAGCCTGGGGCCTCAGTG | | VKKPGASVKV | | | |
| | | SVKVSCKA | | | | EWMG | | QKFQG | | STVYM | | HPLPRF | | | | AAGGTTTCCTGCAAGGCATCTGGATAC | | SCKASGYTFT | | | |
| | | SG | | | | | | | | ELSSL | | | | | | ACCTTCACCAACTACTATATGCTCTGG | | NYYMLWVRQV | | | |
| | | | | | | | | | | RSEDT | | | | | | GTGCGCCAGGTCCCTGGACAAGGGCTT | | PGQGLEWMGL | | | |
| | | | | | | | | | | AMYYC | | | | | | GAGTGGATGGGATTAATCAACCCTAGT | | INPSGGSTSY | | | |
| | | | | | | | | | | | | | | | | GGTGGTAGCACAAGCTACGCACAGAAG | | AQKFQGRVTM | | | |
| | | | | | | | | | | | | | | | | TTCCAGGGCAGAGTCACCATGACCAGG | | TRDTSTSTVY | | | |
| | | | | | | | | | | | | | | | | GACACGTCCACGAGCACAGTCTACATG | | MELSSLRSED | | | |
| | | | | | | | | | | | | | | | | GAGCTGAGCAGCCTGAGATCTGAGGAC | | TAMYYCARDP | | | |
| | | | | | | | | | | | | | | | | ACGGCCATGTATTACTGTGCGAGAGAT | | GVVDPQYPHP | | | |
| | | | | | | | | | | | | | | | | CCGGGTGTGGTGGACCCACAATACCCC | | LPRFWGQGTL | | | |
| | | | | | | | | | | | | | | | | CACCCCCTGCCTCGGTTCTGGGGCCAG | | VTVSS | | | |
| | | | | | | | | | | | | | | | | GGAACCCTGGTCACCGTCTCCTCA | | | | | |
| ADI- | IGHV1- | QVQLVQSG | 177 | GTFSSYS | 407 | WVRQA | 433 | TIIPIL | 744 | RVTIT | 987 | AREYCS | 1248 | WGQGTL | 1286 | CAGGTGCAGCTGGTGCAGTCTGGGGCT | 1548 | QVQLVQSGAE | 1831 | | |
| 75643 | 69 | AEVRVPGS | | IT | | PGQGL | | GVANYA | | ADKST | | GDSCRT | | VTVSS | | GAGGTGAGGGTGCCTGGGTCCTCGGTG | | VRVPGSSVKV | | | |
| | | SVKVSCKA | | | | EWMG | | PKFQG | | STAYM | | VNWFDP | | | | AAGGTCTCCTGCAAGGCTTCTGGAGGC | | SCKASGGTFS | | | |
| | | SG | | | | | | | | EVTSL | | | | | | ACCTTCAGTAGTTATAGTATCACCTGG | | SYSITWVRQA | | | |
| | | | | | | | | | | TSEDT | | | | | | GTGCGACAGGCCCCTGGACAAGGGCTT | | PGQGLEWMGT | | | |
| | | | | | | | | | | AIYYC | | | | | | GAGTGGATGGGAACGATCATCCCTATC | | IIPILGVANY | | | |
| | | | | | | | | | | | | | | | | CTTGGCGTAGCAAACTACGCACCGAAG | | APKFQGRVTI | | | |
| | | | | | | | | | | | | | | | | TTCCAGGGCAGAGTCACCATTACCGCG | | TADKSTSTAY | | | |
| | | | | | | | | | | | | | | | | GACAAATCCACGAGCACAGCCTACATG | | MEVTSLTSED | | | |
| | | | | | | | | | | | | | | | | GAGGTGACCAGCCTGACATCTGAGGAC | | TAIYYCAREY | | | |
| | | | | | | | | | | | | | | | | ACGGCCATATATTACTGTGCGAGAGAA | | CSGDSCRTVN | | | |
| | | | | | | | | | | | | | | | | TATTGTAGTGGTGACAGCTGCCGAACC | | WFDPWGQGTL | | | |
| | | | | | | | | | | | | | | | | GTTAACTGGTTCGACCCCTGGGGCCAG | | VTVSS | | | |
| | | | | | | | | | | | | | | | | GGAACCCTGGTCACCGTCTCCTCA | | | | | |
| ADI- | IGHV4- | QVQLQESG | 67 | GSINNYY | 408 | WIRQP | 445 | YVYFSG | 745 | RVAIS | 988 | ARGLTY | 1249 | WGQGTT | 1287 | CAGGTGCAGCTGCAGGAGTCGGGCCCA | 1549 | QVQLQESGPG | 1832 | | |
| 75740 | 59 | PGLVKPSE | | WN | | PGKGL | | STNYNP | | LDRSK | | NGYDVE | | VTVSS | | GGACTGGTGAAGCCTTCGGAGACCCTG | | LVKPSETLSL | | | |
| | | TLSLTCTV | | | | EWIG | | SLKS | | KQFSL | | AFDI | | | | TCCCTCACCTGCACTGTCTCTGGTGGC | | TCTVSGGSIN | | | |
| | | SG | | | | | | | | KLTSV | | | | | | TCCATCAATAATTACTACTGGAACTGG | | NYYWNWIRQP | | | |
| | | | | | | | | | | TAADT | | | | | | ATCCGGCAGCCCCCAGGGAAGGGACTG | | PGKGLEWIGY | | | |
| | | | | | | | | | | AVYYC | | | | | | GAGTGGATTGGGTATGTCTATTTCAGT | | VYFSGSTNYN | | | |
| | | | | | | | | | | | | | | | | GGGAGCACCAATTACAACCCCTCCCTC | | PSLKSRVAIS | | | |
| | | | | | | | | | | | | | | | | AAGAGTCGAGTCGCCATATCACTAGAC | | LDRSKKQFSL | | | |
| | | | | | | | | | | | | | | | | AGGTCCAAGAAGCAGTTCTCCCTGAAG | | KLTSVTAADT | | | |
| | | | | | | | | | | | | | | | | CTGACCTCTGTGACCGCTGCGGACACG | | AVYYCARGLT | | | |
| | | | | | | | | | | | | | | | | GCCGTGTATTACTGTGCAAGGGGCCTT | | YNGYDVEAFD | | | |
| | | | | | | | | | | | | | | | | ACATATAATGGCTACGATGTAGAAGCT | | IWGQGTTVTV | | | |
| | | | | | | | | | | | | | | | | TTTGATATTTGGGGCCAAGGGACCACG | | SS | | | |
| | | | | | | | | | | | | | | | | GTCACCGTCTCCTCA | | | | | |
| ADI- | IGHV4- | QVQLQQSG | 178 | GSVNTWS | 409 | WIRQR | 512 | HISYSG | 746 | RIAMS | 989 | ARLVRG | 1250 | WGQGTT | 1287 | CAGGTGCAGCTGCAGCAGTCTGGCCCA | 1550 | QVQLQQSGPG | 1833 | | |
| 75720 | 31 | PGLLKPSQ | | YYWG | | PGKGL | | STYYNP | | VDTSK | | GLSYGS | | VTVSS | | GGACTGCTGAAGCCTTCACAGACCCTG | | LLKPSQTLSL | | | |
| | | TLSLTCTV | | | | EWIG | | SLKS | | NQFSL | | DAFDL | | | | TCCCTCACCTGCACTGTCTCTGGTGGC | | TCTVSGGSVN | | | |
| | | SG | | | | | | | | KLRSV | | | | | | TCCGTCAATACTTGGAGTTACTACTGG | | TWSYYWGWIR | | | |
| | | | | | | | | | | TVADT | | | | | | GGCTGGATCCGCCAGCGCCCAGGGAAG | | QRPGKGLEWI | | | |
| | | | | | | | | | | AVYYC | | | | | | GGCCTGGAGTGGATTGGGCACATCTCT | | GHISYSGSTY | | | |
| | | | | | | | | | | | | | | | | TACAGTGGGAGCACCTACTACAACCCG | | YNPSLKSRIA | | | |
| | | | | | | | | | | | | | | | | TCCCTCAAGAGTCGAATTGCCATGTCA | | MSVDTSKNQF | | | |
| | | | | | | | | | | | | | | | | GTAGACACGTCTAAGAACCAGTTCTCC | | SLKLRSVTVA | | | |
| | | | | | | | | | | | | | | | | CTGAAACTGAGGTCTGTGACGGTCGCG | | DTAVYYCARL | | | |
| | | | | | | | | | | | | | | | | GACACGGCCGTGTATTACTGTGCGAGA | | VRGGLSYGSD | | | |
| | | | | | | | | | | | | | | | | CTTGTGAGGGGTGGACTCAGCTATGGC | | AFDLWGQGTT | | | |
| | | | | | | | | | | | | | | | | TCTGACGCTTTTGATCTCTGGGGCCAA | | VTVSS | | | |
| | | | | | | | | | | | | | | | | GGGACCACGGTCACCGTCTCCTCA | | | | | |
| ADI- | IGHV3- | QVQLVQSG | 179 | FTFSSYA | 199 | WVRQA | 431 | VIWNDG | 747 | RFSIS | 990 | LREYQL | 1251 | WGKGTT | 1291 | CAGGTGCAGCTGGTGCAGTCTGGGGGA | 1551 | QVQLVQSGGG | 1834 | | |
| 75794 | 33 | GGVVQPGR | | MH | | PGKGL | | SNKYYT | | RDDSK | | APPYYF | | VTVSS | | GGCGTGGTCCAGCCTGGGAGGTCCCTG | | VVQPGRSLRL | | | |
| | | SLRLSCVG | | | | EWVA | | DSVKG | | NTLDL | | MDV | | | | AGACTCTCCTGTGTAGGGTCTGGATTC | | SCVGSGFTFS | | | |
| | | SG | | | | | | | | QMNSL | | | | | | ACCTTCAGTAGCTATGCCATGCACTGG | | SYAMHWVRQA | | | |
| | | | | | | | | | | RAEDT | | | | | | GTCCGCCAGGCTCCAGGCAAGGGGCTG | | PGKGLEWVAV | | | |
| | | | | | | | | | | AVYYC | | | | | | GAGTGGGTGGCAGTTATTTGGAATGAT | | IWNDGSNKYY | | | |
| | | | | | | | | | | | | | | | | GGAAGTAATAAATATTATACAGACTCC | | TDSVKGRFSI | | | |
| | | | | | | | | | | | | | | | | GTGAAGGGCCGATTCAGTATCTCCAGA | | SRDDSKNTLD | | | |
| | | | | | | | | | | | | | | | | GACGATTCCAAGAACACGCTGGATCTG | | LQMNSLRAED | | | |
| | | | | | | | | | | | | | | | | CAAATGAACAGCCTGAGGGCCGAGGAC | | TAVYYCLREY | | | |
| | | | | | | | | | | | | | | | | ACGGCTGTCTATTATTGTTTGAGAGAA | | QLAPPYYFMD | | | |
| | | | | | | | | | | | | | | | | TATCAGCTTGCCCCCCCCTACTACTTC | | VWGKGTTVTV | | | |
| | | | | | | | | | | | | | | | | ATGGACGTCTGGGGCAAAGGGACCACG | | SS | | | |
| | | | | | | | | | | | | | | | | GTCACCGTCTCCTCA | | | | | |
| ADI- | IGHV3- | QVQLVQSG | 165 | FTFSRYA | 410 | WVRQA | 431 | VISFRE | 748 | RFTVS | 991 | ARGDIL | 1252 | WGQGTT | 1287 | CAGGTGCAGCTGGTGCAGTCTGGGGGA | 1552 | QVQLVQSGGG | 1835 | | |
| 75577 | 30 | GGVVQPGR | | MH | | PGKGL | | SDKNYA | | RDNSK | | TGYPTS | | VTVSS | | GGCGTGGTCCAGCCTGGGAGGTCCCTG | | VVQPGRSLRL | | | |
| | | SLRLSCAA | | | | EWVA | | DSVKG | | NMLYL | | KDAFDI | | | | AGACTCTCCTGTGCAGCCTCTGAATTC | | SCAASEFTFS | | | |
| | | SE | | | | | | | | QMNSL | | | | | | ACCTTCAGTCGCTATGCTATGCACTGG | | RYAMHWVRQA | | | |
| | | | | | | | | | | RAEDT | | | | | | GTCCGCCAGGCTCCAGGCAAGGGACTG | | PGKGLEWVAV | | | |
| | | | | | | | | | | AVYYC | | | | | | GAGTGGGTGGCTGTTATATCTTTTCGT | | ISFRESDKNY | | | |
| | | | | | | | | | | | | | | | | GAAAGTGATAAAAATTACGCAGACTCC | | ADSVKGRFTV | | | |
| | | | | | | | | | | | | | | | | GTGAAGGGCCGATTCACCGTCTCCAGA | | SRDNSKNMLY | | | |
| | | | | | | | | | | | | | | | | GACAATTCCAAGAATATGCTGTATCTG | | LQMNSLRAED | | | |
| | | | | | | | | | | | | | | | | CAAATGAACAGCCTGAGAGCTGAGGAC | | TAVYYCARGD | | | |
| | | | | | | | | | | | | | | | | ACGGCTGTATATTACTGTGCGCGAGGC | | ILTGYPTSKD | | | |
| | | | | | | | | | | | | | | | | GATATTTTGACTGGCTACCCGACATCA | | AFDIWGQGTT | | | |
| | | | | | | | | | | | | | | | | AAGGATGCTTTTGATATCTGGGGCCAA | | VTVSS | | | |
| | | | | | | | | | | | | | | | | GGGACCACGGTCACCGTCTCCTCA | | | | | |
| ADI- | IGHV3- | QVTLQQSG | 180 | FTFDDYA | 214 | WVRQA | 437 | GITWNS | 749 | RFIIS | 992 | AKDILS | 1253 | WGQGTT | 1287 | CAGGTCACGTTGCAGCAGTCTGGGGGA | 1553 | QVTLQQSGGG | 1836 | | |
| 75850 | 9 | GGLVQPGR | | MH | | PGKGL | | GIIGYA | | RDNAK | | ERHYFD | | VTVSS | | GGCTTGGTACAGCCTGGCAGGTCCCTG | | LVQPGRSLRL | | | |
| | | SLRLSCAA | | | | EWVS | | DSVKG | | NSLFL | | SSGSYG | | | | AGACTCTCCTGTGCAGCCTCTGGATTC | | SCAASGFTFD | | | |
| | | SG | | | | | | | | QMYSL | | MDV | | | | ACCTTTGATGATTATGCCATGCACTGG | | DYAMHWVRQA | | | |
| | | | | | | | | | | RAEDT | | | | | | GTCCGGCAAGCTCCAGGGAAGGGCCTG | | PGKGLEWVSG | | | |
| | | | | | | | | | | ALYFC | | | | | | GAGTGGGTCTCAGGTATTACTTGGAAT | | ITWNSGIIGY | | | |
| | | | | | | | | | | | | | | | | AGTGGTATCATAGGCTATGCGGACTCT | | ADSVKGRFII | | | |
| | | | | | | | | | | | | | | | | GTGAAGGGCCGATTCATCATCTCCAGA | | SRDNAKNSLF | | | |
| | | | | | | | | | | | | | | | | GACAACGCCAAGAACTCCCTGTTTCTT | | LQMYSLRAED | | | |
| | | | | | | | | | | | | | | | | CAAATGTACAGTCTGAGAGCTGAGGAC | | TALYFCAKDI | | | |
| | | | | | | | | | | | | | | | | ACGGCCTTGTATTTCTGTGCAAAAGAT | | LSERHYFDSS | | | |
| | | | | | | | | | | | | | | | | ATTTTAAGCGAGCGTCATTATTTTGAT | | GSYGMDVWGQ | | | |
| | | | | | | | | | | | | | | | | AGTAGTGGTTCCTACGGTATGGACGTC | | GTTVTVSS | | | |
| | | | | | | | | | | | | | | | | TGGGGCCAAGGGACCACGGTCACCGTC | | | | | |
| | | | | | | | | | | | | | | | | TCCTCA | | | | | |
| ADI- | IGHV1- | QVQLVQSG | 50 | GTFSSYP | 379 | WVRQA | 433 | RIIPIL | 750 | RVTIT | 832 | ARDIGY | 1254 | WGRGTL | 1288 | CAGGTCCAGCTTGTGCAGTCTGGGGCT | 1554 | QVQLVQSGAE | 1837 | | |
| 75770 | 69 | AEVKKPGS | | IS | | PGQGL | | GIPNYT | | ADKST | | SGYQSA | | VTVSS | | GAGGTGAAGAAGCCTGGGTCCTCGGTG | | VKKPGSSVKV | | | |
| | | SVKVSCKA | | | | EWMG | | QKFQG | | STAYM | | EYFDL | | | | AAGGTCTCCTGCAAGGCTTCTGGAGGC | | SCKASGGTFS | | | |
| | | SG | | | | | | | | ELSSL | | | | | | ACCTTCAGCAGCTATCCTATCAGCTGG | | SYPISWVRQA | | | |
| | | | | | | | | | | RSEDT | | | | | | GTGCGACAGGCCCCTGGACAAGGGCTT | | PGQGLEWMGR | | | |
| | | | | | | | | | | AVYYC | | | | | | GAGTGGATGGGAAGGATCATCCCTATC | | IIPILGIPNY | | | |
| | | | | | | | | | | | | | | | | CTTGGTATACCAAACTACACACAGAAG | | TQKFQGRVTI | | | |
| | | | | | | | | | | | | | | | | TTCCAGGGCAGAGTCACCATTACCGCG | | TADKSTSTAY | | | |
| | | | | | | | | | | | | | | | | GACAAATCCACGAGCACAGCCTACATG | | MELSSLRSED | | | |
| | | | | | | | | | | | | | | | | GAGCTGAGCAGCCTGAGATCTGAGGAC | | TAVYYCARDI | | | |
| | | | | | | | | | | | | | | | | ACGGCCGTGTATTACTGTGCGAGAGAT | | GYSGYQSAEY | | | |
| | | | | | | | | | | | | | | | | ATTGGATATAGTGGCTACCAATCGGCT | | FDLWGRGTLV | | | |
| | | | | | | | | | | | | | | | | GAATACTTCGATCTCTGGGGCCGTGGC | | TVSS | | | |
| | | | | | | | | | | | | | | | | ACCCTGGTCACCGTCTCCTCA | | | | | |
| ADI- | IGHV3- | QVQLVQSG | 181 | FTVSSNY | 369 | WVRQA | 437 | VIYSGG | 659 | RFTIS | 777 | ARDFYG | 1255 | WGQGTL | 1286 | CAGGTCCAGCTGGTGCAGTCTGGGGGA | 1555 | QVQLVQSGGG | 1838 | | |
| 75799 | 66 | GGLVQPGG | | MN | | PGKGL | | STYYAD | | RDNSK | | SGSPND | | VTVSS | | GGCTTGGTCCAGCCTGGGGGGTCCCTG | | LVQPGGSLRL | | | |
| | | SLRLSCVV | | | | EWVS | | SVKG | | NTLYL | | SHDY | | | | AGACTCTCCTGTGTAGTCTCTGGATTC | | SCVVSGFTVS | | | |
| | | SG | | | | | | | | QMNSL | | | | | | ACCGTCAGTAGCAACTACATGAACTGG | | SNYMNWVRQA | | | |
| | | | | | | | | | | RAEDT | | | | | | GTCCGCCAGGCTCCAGGGAAGGGGCTG | | PGKGLEWVSV | | | |
| | | | | | | | | | | AVYYC | | | | | | GAGTGGGTCTCAGTTATTTATAGCGGT | | IYSGGSTYYA | | | |
| | | | | | | | | | | | | | | | | GGTAGCACATACTACGCAGACTCCGTG | | DSVKGRFTIS | | | |
| | | | | | | | | | | | | | | | | AAGGGCAGATTCACCATCTCCAGAGAC | | RDNSKNTLYL | | | |
| | | | | | | | | | | | | | | | | AATTCCAAGAACACGCTGTATCTTCAA | | QMNSLRAEDT | | | |
| | | | | | | | | | | | | | | | | ATGAACAGCCTGAGAGCCGAGGACACG | | AVYYCARDFY | | | |
| | | | | | | | | | | | | | | | | GCTGTGTATTATTGTGCGAGAGATTTT | | GSGSPNDSHD | | | |
| | | | | | | | | | | | | | | | | TATGGTTCGGGGAGTCCTAATGACTCG | | YWGQGTLVTV | | | |
| | | | | | | | | | | | | | | | | CATGACTACTGGGGCCAGGGAACCCTG | | SS | | | |
| | | | | | | | | | | | | | | | | GTCACCGTCTCCTCA | | | | | |
| ADI- | IGHV1- | QVQLVQSG | 35 | YTFTTYG | 411 | WVRQA | 433 | WISAFN | 751 | RVTMT | 993 | ARGNCG | 1256 | WGKGTT | 1291 | CAGGTGCAGCTGGTGCAGTCTGGAGCT | 1556 | QVQLVQSGAE | 1839 | | |
| 75759 | 18 | AEVKKPGA | | IT | | PGQGL | | DNTNYA | | TDTST | | PNCAYY | | VTVSS | | GAGGTGAAGAAGCCTGGGGCCTCAGTG | | VKKPGASVKV | | | |
| | | SVKVSCKA | | | | EWMG | | QNFQG | | STAYM | | HYMDV | | | | AAGGTCTCCTGCAAGGCTTCTGGTTAC | | SCKASGYTFT | | | |
| | | SG | | | | | | | | ELRSL | | | | | | ACCTTTACCACTTATGGAATCACCTGG | | TYGITWVRQA | | | |
| | | | | | | | | | | RSDDT | | | | | | GTGCGACAGGCCCCTGGACAAGGGCTT | | PGQGLEWMGW | | | |
| | | | | | | | | | | AVYYC | | | | | | GAGTGGATGGGATGGATCAGCGCTTTC | | ISAFNDNTNY | | | |
| | | | | | | | | | | | | | | | | AACGATAACACAAACTATGCACAGAAT | | AQNFQGRVTM | | | |
| | | | | | | | | | | | | | | | | TTCCAGGGCAGAGTCACCATGACCACA | | TTDTSTSTAY | | | |
| | | | | | | | | | | | | | | | | GACACATCCACGAGCACAGCCTACATG | | MELRSLRSDD | | | |
| | | | | | | | | | | | | | | | | GAGTTGAGGAGCCTGAGATCTGACGAC | | TAVYYCARGN | | | |
| | | | | | | | | | | | | | | | | ACGGCCGTGTATTACTGTGCGAGGGGA | | CGPNCAYYHY | | | |
| | | | | | | | | | | | | | | | | AATTGTGGTCCTAATTGCGCCTATTAT | | MDVWGKGTTV | | | |
| | | | | | | | | | | | | | | | | CACTACATGGACGTCTGGGGCAAAGGG | | TVSS | | | |
| | | | | | | | | | | | | | | | | ACCACGGTCACCGTCTCCTCA | | | | | |
| ADI- | IGHV3- | QVQLVQSG | 17 | FTFEDYA | 208 | WIRQS | 513 | GISWNG | 752 | RFTIS | 994 | AKDEGS | 1257 | WGQGTL | 1286 | CAGGTGCAGCTGGTGCAGTCTGGGGGA | 1557 | QVQLVQSGGG | 1840 | | |
| 75803 | 9 | GGLVQPGR | | MH | | PGKGL | | GIIGYA | | RDNDK | | EGDQLP | | VTVSS | | GGCTTGGTACAGCCTGGCAGGTCCCTG | | LVQPGRSLRL | | | |
| | | SLRLSCAA | | | | EWVS | | DSVKG | | NSLYL | | LDY | | | | AGACTCTCCTGTGCAGCCTCTGGATTC | | SCAASGFTFE | | | |
| | | SG | | | | | | | | HIRSL | | | | | | ACCTTTGAGGATTATGCCATGCACTGG | | DYAMHWIRQS | | | |
| | | | | | | | | | | KAEDT | | | | | | ATCCGGCAATCTCCAGGGAAGGGCCTG | | PGKGLEWVSG | | | |
| | | | | | | | | | | ALYYC | | | | | | GAGTGGGTCTCAGGTATTAGTTGGAAT | | ISWNGGIIGY | | | |
| | | | | | | | | | | | | | | | | GGTGGTATCATAGGCTATGCGGACTCT | | ADSVKGRFTI | | | |
| | | | | | | | | | | | | | | | | GTGAAGGGCCGATTCACCATCTCCAGA | | SRDNDKNSLY | | | |
| | | | | | | | | | | | | | | | | GACAACGACAAGAACTCCCTGTATCTG | | LHIRSLKAED | | | |
| | | | | | | | | | | | | | | | | CATATTAGGAGTCTGAAAGCTGAGGAC | | TALYYCAKDE | | | |
| | | | | | | | | | | | | | | | | ACGGCCTTGTATTACTGTGCAAAAGAT | | GSEGDQLPLD | | | |
| | | | | | | | | | | | | | | | | GAAGGATCTGAGGGGGACCAGCTGCCC | | YWGQGTLVTV | | | |
| | | | | | | | | | | | | | | | | CTTGACTACTGGGGCCAGGGAACCCTG | | SS | | | |
| | | | | | | | | | | | | | | | | GTCACCGTCTCCTCA | | | | | |
| ADI- | IGHV1- | RCTLQQSG | 182 | GTFSGFP | 412 | WVRQA | 482 | RIIPIL | 739 | RVTLT | 995 | ARDAGY | 1258 | WGQGAL | 1285 | AGGTGCACGCTGCAGCAGTCTGGGGCT | 1558 | RCTLQQSGAE | 1841 | | |
| 75710 | 69 | AEVKKPGS | | IS | | PEQGL | | GVADYA | | ADKST | | SGSGSS | | VTVSS | | GAGGTGAAGAAGCCTGGGTCCTCGGTG | | VKKPGSSVKV | | | |
| | | SVKVSCKA | | | | EWMG | | QKFQG | | GTAYM | | YYFDY | | | | AAGGTCTCCTGCAAGGCTTCTGGAGGC | | SCKASGGTFS | | | |
| | | SG | | | | | | | | ELSSL | | | | | | ACCTTCAGCGGTTTTCCCATCAGCTGG | | GFPISWVRQA | | | |
| | | | | | | | | | | RSEDT | | | | | | GTGCGACAGGCCCCTGAACAAGGGCTT | | PEQGLEWMGR | | | |
| | | | | | | | | | | AVYYC | | | | | | GAGTGGATGGGAAGGATCATCCCTATC | | IIPILGVADY | | | |
| | | | | | | | | | | | | | | | | CTTGGTGTAGCAGACTACGCACAGAAA | | AQKFQGRVTL | | | |
| | | | | | | | | | | | | | | | | TTCCAGGGCAGAGTCACGCTTACCGCG | | TADKSTGTAY | | | |
| | | | | | | | | | | | | | | | | GACAAATCCACGGGCACAGCGTACATG | | MELSSLRSED | | | |
| | | | | | | | | | | | | | | | | GAGCTGAGCAGCCTGAGGTCTGAAGAC | | TAVYYCARDA | | | |
| | | | | | | | | | | | | | | | | ACGGCCGTGTATTACTGTGCGAGAGAT | | GYSGSGSSYY | | | |
| | | | | | | | | | | | | | | | | GCGGGTTACTCTGGTTCGGGGAGTTCT | | FDYWGQGALV | | | |
| | | | | | | | | | | | | | | | | TACTACTTTGACTACTGGGGCCAGGGA | | TVSS | | | |
| | | | | | | | | | | | | | | | | GCCCTGGTCACCGTCTCCTCA | | | | | |
| ADI- | IGHV3- | QVQLQESG | 183 | FTFDDYA | 336 | WVRQA | 437 | GATWNS | 753 | RFIIS | 996 | AKDYYG | 1259 | WGPGTT | 1295 | CAGGTCCAGCTGCAGGAGTCTGGGGGA | 1559 | QVQLQESGGG | 1842 | | |
| 75605 | 9 | GGLVQPGR | | IH | | PGKGL | | GTIGYA | | RDNAK | | SGSNSP | | VTVSS | | GGCTTGGTACAGCCTGGCAGGTCCCTG | | LVQPGRSLRL | | | |
| | | SLRLSCVA | | | | EWVS | | DSVEG | | NSLYL | | RDHYYG | | | | AGACTCTCCTGTGTAGCCTCTGGATTC | | SCVASGFTFD | | | |
| | | SG | | | | | | | | QMNSL | | MDV | | | | ACCTTTGATGATTATGCCATACACTGG | | DYAIHWVRQA | | | |
| | | | | | | | | | | RVEDT | | | | | | GTCCGGCAAGCTCCAGGGAAGGGCCTG | | PGKGLEWVSG | | | |
| | | | | | | | | | | ALYYC | | | | | | GAGTGGGTCTCAGGTGCTACTTGGAAT | | ATWNSGTIGY | | | |
| | | | | | | | | | | | | | | | | AGTGGTACCATAGGCTATGCGGACTCT | | ADSVEGRFII | | | |
| | | | | | | | | | | | | | | | | GTCGAGGGCCGATTCATCATCTCCAGA | | SRDNAKNSLY | | | |
| | | | | | | | | | | | | | | | | GACAACGCCAAGAACTCCCTGTATCTG | | LQMNSLRVED | | | |
| | | | | | | | | | | | | | | | | CAGATGAACAGTCTGAGAGTTGAGGAC | | TALYYCAKDY | | | |
| | | | | | | | | | | | | | | | | ACGGCCTTGTATTATTGTGCAAAAGAT | | YGSGSNSPRD | | | |
| | | | | | | | | | | | | | | | | TACTATGGTTCAGGGAGCAACAGTCCC | | HYYGMDVWGP | | | |
| | | | | | | | | | | | | | | | | AGGGACCACTATTACGGTATGGACGTC | | GTTVTVSS | | | |
| | | | | | | | | | | | | | | | | TGGGGCCCAGGGACCACGGTCACCGTC | | | | | |
| | | | | | | | | | | | | | | | | TCCTCA | | | | | |
| ADI- | IGHV1- | QVQLVQSG | 184 | GTFSTYT | 413 | WVRQA | 514 | GIIPLI | 754 | RVSIT | 997 | GGSSDP | 1260 | WGQGTL | 1286 | CAGGTGCAGCTGGTGCAGTCTGGGGCT | 1560 | QVQLVQSGAE | 1843 | | |
| 75680 | 69 | AEVKKPGS | | FS | | SGQGL | | GRANYA | | ADEPT | | PLRFWT | | VTVSS | | GAGGTGAAGAAGCCTGGGTCCTCGGTG | | VKKPGSSVKV | | | |
| | | SVKVSCKP | | | | EWMG | | QKFQG | | STVYL | | LDN | | | | AAGGTCTCCTGCAAGCCTGTTGGAGGC | | SCKPVGGTFS | | | |
| | | VG | | | | | | | | ELSRL | | | | | | ACCTTCAGCACCTATACTTTCAGTTGG | | TYTFSWVRQA | | | |
| | | | | | | | | | | KSEDT | | | | | | GTGCGACAGGCCTCTGGACAAGGGCTT | | SGQGLEWMGG | | | |
| | | | | | | | | | | AVYYC | | | | | | GAGTGGATGGGAGGGATTATCCCTTTG | | IIPLIGRANY | | | |
| | | | | | | | | | | | | | | | | ATTGGTAGAGCCAACTACGCACAGAAG | | AQKFQGRVSI | | | |
| | | | | | | | | | | | | | | | | TTCCAGGGCAGAGTCTCGATTACCGCG | | TADEPTSTVY | | | |
| | | | | | | | | | | | | | | | | GACGAACCCACGAGCACAGTCTATCTG | | LELSRLKSED | | | |
| | | | | | | | | | | | | | | | | GAACTGAGCAGACTGAAATCTGAAGAC | | TAVYYCGGSS | | | |
| | | | | | | | | | | | | | | | | ACGGCCGTCTATTATTGTGGGGGATCC | | DPPLRFWTLD | | | |
| | | | | | | | | | | | | | | | | TCCGACCCTCCACTACGATTTTGGACT | | NWGQGTLVTV | | | |
| | | | | | | | | | | | | | | | | CTTGACAACTGGGGCCAGGGAACCCTG | | SS | | | |
| | | | | | | | | | | | | | | | | GTCACCGTCTCCTCA | | | | | |
| ADI- | IGHV1- | QVQLVQSG | 184 | GTFSTYT | 414 | WVRQA | 514 | GIIPLI | 754 | RVSIT | 998 | GGSSDP | 1260 | WGQGTL | 1286 | CAGGTGCAGCTGGTGCAGTCTGGGGCT | 1561 | QVQLVQSGAE | 1844 | | |
| 75691 | 69 | AEVKKPGS | | FN | | SGQGL | | GRANYA | | ADEPT | | PLRFWT | | VTVSS | | GAGGTGAAGAAGCCTGGGTCCTCGGTG | | VKKPGSSVKV | | | |
| | | SVKVSCKP | | | | EWMG | | QKFQG | | STVYI | | LDN | | | | AAGGTCTCCTGCAAGCCTGTTGGAGGC | | SCKPVGGTFS | | | |
| | | VG | | | | | | | | ELSRL | | | | | | ACCTTCAGCACCTATACTTTCAACTGG | | TYTFNWVRQA | | | |
| | | | | | | | | | | KSEDT | | | | | | GTGCGACAGGCCTCTGGACAAGGGCTT | | SGQGLEWMGG | | | |
| | | | | | | | | | | AVYYC | | | | | | GAGTGGATGGGAGGGATCATCCCTTTA | | IIPLIGRANY | | | |
| | | | | | | | | | | | | | | | | ATTGGTAGAGCCAACTACGCACAGAAG | | AQKFQGRVSI | | | |
| | | | | | | | | | | | | | | | | TTCCAGGGCAGAGTCTCGATTACCGCG | | TADEPTSTVY | | | |
| | | | | | | | | | | | | | | | | GACGAACCCACCAGCACAGTCTACATA | | IELSRLKSED | | | |
| | | | | | | | | | | | | | | | | GAACTGAGCAGACTGAAATCTGAAGAC | | TAVYYCGGSS | | | |
| | | | | | | | | | | | | | | | | ACGGCCGTGTATTACTGTGGGGGATCC | | DPPLRFWTLD | | | |
| | | | | | | | | | | | | | | | | TCCGACCCTCCACTACGATTTTGGACT | | NWGQGTLVTV | | | |
| | | | | | | | | | | | | | | | | CTTGACAACTGGGGCCAGGGAACCCTG | | SS | | | |
| | | | | | | | | | | | | | | | | GTCACCGTCTCCTCA | | | | | |
| ADI- | IGHV3- | QVQLVQSG | 185 | FTFGDYA | 415 | WVRQA | 432 | FIRSKA | 755 | RFTIS | 999 | TREGGV | 1261 | WGQGTL | 1286 | CAGGTGCAGCTGGTGCAGTCTGGGGGA | 1562 | QVQLVQSGGG | 1845 | | |
| 75573 | 49 | GGLVQPGR | | MN | | PGKGL | | YGGTTE | | RDDSK | | GVYRDW | | VTVSS | | GGCTTGGTACAGCCAGGGCGGTCCCTG | | LVQPGRSLRL | | | |
| | | SLRLSCTA | | | | EWVG | | YAASVK | | SIAYL | | FDP | | | | AGACTCTCCTGTACAGCTTCTGGATTC | | SCTASGFTFG | | | |
| | | SG | | | | | | G | | QMNSL | | | | | | ACCTTTGGTGATTATGCTATGAACTGG | | DYAMNWVRQA | | | |
| | | | | | | | | | | KTEDT | | | | | | GTCCGCCAGGCTCCAGGGAAGGGGCTG | | PGKGLEWVGF | | | |
| | | | | | | | | | | AVYYC | | | | | | GAGTGGGTAGGTTTCATTAGAAGCAAA | | IRSKAYGGTT | | | |
| | | | | | | | | | | | | | | | | GCTTATGGTGGGACAACAGAATACGCC | | EYAASVKGRF | | | |
| | | | | | | | | | | | | | | | | GCGTCTGTGAAAGGCAGATTCACCATC | | TISRDDSKSI | | | |
| | | | | | | | | | | | | | | | | TCAAGAGATGATTCCAAAAGCATCGCC | | AYLQMNSLKT | | | |
| | | | | | | | | | | | | | | | | TATCTGCAAATGAACAGCCTGAAAACC | | EDTAVYYCTR | | | |
| | | | | | | | | | | | | | | | | GAGGACACAGCCGTGTATTACTGTACT | | EGGVGVYRDW | | | |
| | | | | | | | | | | | | | | | | AGAGAGGGGGGGGTGGGGGTATATCGG | | FDPWGQGTLV | | | |
| | | | | | | | | | | | | | | | | GACTGGTTCGACCCCTGGGGCCAGGGA | | TVSS | | | |
| | | | | | | | | | | | | | | | | ACCCTGGTCACCGTCTCCTCA | | | | | |
| ADI- | IGHV1- | QVQLQQSG | 86 | GTFNSYA | 416 | WVRQA | 433 | GIITVL | 756 | RVTIV | 1000 | ARVGEN | 1262 | WGQGTT | 1287 | CAGGTCCAGCTGCAGCAGTCTGGGGCT | 1563 | QVQLQQSGAE | 1846 | | |
| 75580 | 69 | AEVKKPGS | | VS | | PGQGL | | RTTKYA | | ADEST | | LVSLGG | | VTVSS | | GAGGTGAAGAAGCCCGGGTCCTCGGTG | | VKKPGSSVKV | | | |
| | | SVKVSCKA | | | | EWMG | | QRFQG | | GTAYM | | GDFHYY | | | | AAGGTCTCCTGCAAGGCTTCTGGAGGC | | SCKASGGTFN | | | |
| | | SG | | | | | | | | ELSSL | | GMDV | | | | ACCTTCAACAGCTATGCTGTCAGTTGG | | SYAVSWVRQA | | | |
| | | | | | | | | | | RFEDT | | | | | | GTGCGACAGGCCCCTGGACAAGGGCTT | | PGQGLEWMGG | | | |
| | | | | | | | | | | AVYYC | | | | | | GAGTGGATGGGAGGCATCATCACTGTC | | IITVLRTTKY | | | |
| | | | | | | | | | | | | | | | | CTTCGTACAACAAAGTATGCACAGAGG | | AQRFQGRVTI | | | |
| | | | | | | | | | | | | | | | | TTCCAGGGCAGAGTCACGATTGTTGCG | | VADESTGTAY | | | |
| | | | | | | | | | | | | | | | | GACGAATCCACGGGCACCGCCTACATG | | MELSSLRFED | | | |
| | | | | | | | | | | | | | | | | GAGCTGAGCAGCCTGAGATTTGAGGAC | | TAVYYCARVG | | | |
| | | | | | | | | | | | | | | | | ACGGCCGTGTACTACTGTGCGAGAGTG | | ENLVSLGGGD | | | |
| | | | | | | | | | | | | | | | | GGGGAGAACCTCGTTAGTCTCGGAGGG | | FHYYGMDVWG | | | |
| | | | | | | | | | | | | | | | | GGGGATTTCCACTACTACGGTATGGAC | | QGTTVTVSS | | | |
| | | | | | | | | | | | | | | | | GTCTGGGGCCAAGGGACCACGGTCACC | | | | | |
| | | | | | | | | | | | | | | | | GTCTCTTCA | | | | | |
| ADI- | IGHV7- | EVQLVQSG | 186 | YSFTSKS | 417 | WVRQA | 433 | WINTNT | 757 | RFVFS | 1001 | ARLVRS | 1263 | WGQGTT | 1287 | GAGGTCCAGCTGGTGCAGTCTGGGTCT | 1564 | EVQLVQSGSE | 1847 | | |
| 75576 | 4-1 | SELKKPGA | | IN | | PGQGL | | GDPTYA | | LDTSV | | TRYYDV | | VTVSS | | GAGTTGAAGAAGCCTGGGGCCTCAGTG | | LKKPGASVKV | | | |
| | | SVKVSCKA | | | | EWMG | | QGFIG | | STAYL | | GGFDI | | | | AAGGTTTCCTGCAAGGCTTCTGGATAC | | SCKASGYSFT | | | |
| | | SG | | | | | | | | QINSL | | | | | | AGCTTCACTAGTAAAAGTATTAATTGG | | SKSINWVRQA | | | |
| | | | | | | | | | | KAEDT | | | | | | GTGCGACAGGCCCCTGGACAAGGGCTT | | PGQGLEWMGW | | | |
| | | | | | | | | | | AVYFC | | | | | | GAGTGGATGGGATGGATCAACACCAAC | | INTNTGDPTY | | | |
| | | | | | | | | | | | | | | | | ACTGGGGACCCAACGTATGCCCAGGGC | | AQGFIGRFVF | | | |
| | | | | | | | | | | | | | | | | TTCATAGGACGGTTTGTCTTCTCCTTG | | SLDTSVSTAY | | | |
| | | | | | | | | | | | | | | | | GACACCTCTGTCAGCACGGCATATCTG | | LQINSLKAED | | | |
| | | | | | | | | | | | | | | | | CAGATCAACAGCCTAAAGGCTGAGGAC | | TAVYFCARLV | | | |
| | | | | | | | | | | | | | | | | ACTGCCGTTTATTTCTGTGCGAGACTC | | RSTRYYDVGG | | | |
| | | | | | | | | | | | | | | | | GTGCGTAGTACCAGGTACTACGACGTG | | FDIWGQGTTV | | | |
| | | | | | | | | | | | | | | | | GGCGGTTTTGATATCTGGGGCCAAGGG | | TVSS | | | |
| | | | | | | | | | | | | | | | | ACCACGGTCACCGTCTCCTCA | | | | | |
| ADI- | IGHV4- | QVQLQQSG | 56 | DSISSGG | 418 | WIRQH | 450 | YIYFTG | 758 | RVIIS | 1002 | ARAGTM | 1264 | WGQGTL | 1286 | CAGGTGCAGCTGCAGCAGTCGGGCCCA | 1565 | QVQLQQSGPG | 1848 | | |
| 75592 | 31 | PGLVKPSQ | | YYWS | | PGKGL | | STDYNP | | VDRFK | | YGDFDY | | VTVSS | | GGACTGGTGAAGCCTTCACAGACCCTG | | LVKPSQTLSL | | | |
| | | TLSLTCTV | | | | EWIG | | SLKS | | NQFSL | | | | | | TCCCTCACCTGCACTGTGTCTGGTGAC | | TCTVSGDSIS | | | |
| | | SG | | | | | | | | KLSSV | | | | | | TCCATCAGCAGTGGTGGTTACTACTGG | | SGGYYWSWIR | | | |
| | | | | | | | | | | SAADT | | | | | | AGCTGGATCCGCCAGCACCCAGGGAAG | | QHPGKGLEWI | | | |
| | | | | | | | | | | AVYYC | | | | | | GGCCTGGAGTGGATTGGGTACATCTAT | | GYIYFTGSTD | | | |
| | | | | | | | | | | | | | | | | TTCACTGGGAGCACCGACTACAACCCG | | YNPSLKSRVI | | | |
| | | | | | | | | | | | | | | | | TCCCTCAAGAGTCGAGTTATCATATCA | | ISVDRFKNQF | | | |
| | | | | | | | | | | | | | | | | GTAGACAGGTTCAAGAACCAGTTTTCA | | SLKLSSVSAA | | | |
| | | | | | | | | | | | | | | | | CTGAAACTGAGTTCTGTGTCTGCCGCG | | DTAVYYCARA | | | |
| | | | | | | | | | | | | | | | | GACACGGCCGTGTATTACTGTGCGAGA | | GTMYGDFDYW | | | |
| | | | | | | | | | | | | | | | | GCCGGGACTATGTATGGGGATTTTGAC | | GQGTLVTVSS | | | |
| | | | | | | | | | | | | | | | | TACTGGGGCCAGGGAACCCTGGTCACC | | | | | |
| | | | | | | | | | | | | | | | | GTCTCCTCA | | | | | |
| ADI- | IGHV2- | QITLKESG | 187 | FSLNTPR | 419 | WIRQP | 441 | HIFSND | 759 | RLTIS | 1003 | ARNMGL | 1265 | WGQGTL | 1286 | CAGATCACGTTGAAGGAGTCTGGTCCT | 1566 | QITLKESGPV | 1849 | | |
| 75597 | 26 | PVLVKPTE | | MGVS | | PGKAL | | EELYST | | RDTSK | | LAVAGI | | VTVSS | | GTGCTGGTGAAACCCACAGAGACCCTC | | LVKPTETLTL | | | |
| | | TLTLTCSV | | | | EWLA | | SLKS | | SQVLL | | QEGWFD | | | | ACGCTGACCTGCTCCGTCTCTGGCTTC | | TCSVSGFSLN | | | |
| | | SG | | | | | | | | TMTNM | | T | | | | TCTCTCAACACTCCTAGAATGGGTGTG | | TPRMGVSWIR | | | |
| | | | | | | | | | | GPADT | | | | | | AGTTGGATCCGTCAGCCCCCCGGGAAG | | QPPGKALEWL | | | |
| | | | | | | | | | | ATYYC | | | | | | GCCCTGGAGTGGCTTGCACACATTTTT | | AHIFSNDEEL | | | |
| | | | | | | | | | | | | | | | | TCGAATGACGAAGAATTGTACAGCACA | | YSTSLKSRLT | | | |
| | | | | | | | | | | | | | | | | TCTCTGAAGAGCAGGCTCACCATCTCC | | ISRDTSKSQV | | | |
| | | | | | | | | | | | | | | | | AGGGACACCTCCAAAAGCCAAGTGCTC | | LLTMTNMGPA | | | |
| | | | | | | | | | | | | | | | | CTTACCATGACCAACATGGGCCCTGCG | | DTATYYCARN | | | |
| | | | | | | | | | | | | | | | | GACACAGCCACTTATTACTGTGCACGG | | MGLLAVAGIQ | | | |
| | | | | | | | | | | | | | | | | AATATGGGTCTTCTGGCAGTGGCTGGT | | EGWFDTWGQG | | | |
| | | | | | | | | | | | | | | | | ATTCAAGAAGGTTGGTTCGACACCTGG | | TLVTVSS | | | |
| | | | | | | | | | | | | | | | | GGCCAGGGAACCCTGGTCACCGTCTCC | | | | | |
| | | | | | | | | | | | | | | | | TCA | | | | | |
| ADI- | IGHV1- | QVQLQQSG | 86 | GTFSSYP | 379 | WVRQA | 433 | SIIPIL | 760 | RVTIN | 1004 | ARDLGY | 1217 | WGQGTL | 1286 | CAGGTACAGCTGCAGCAGTCTGGGGCT | 1567 | QVQLQQSGAE | 1850 | | |
| 75636 | 69 | AEVKKPGS | | IS | | PGQGL | | GVANYA | | ADKST | | SSGGAN | | VTVSS | | GAGGTGAAGAAGCCTGGGTCCTCGGTG | | VKKPGSSVKV | | | |
| | | SVKVSCKA | | | | EWMG | | QKFQG | | STAYM | | YYFDY | | | | AAGGTCTCCTGCAAGGCTTCTGGAGGC | | SCKASGGTFS | | | |
| | | SG | | | | | | | | ELSSL | | | | | | ACCTTCAGCAGCTATCCTATCAGCTGG | | SYPISWVRQA | | | |
| | | | | | | | | | | RSEDT | | | | | | GTGCGACAGGCCCCTGGACAAGGGCTT | | PGQGLEWMGS | | | |
| | | | | | | | | | | AVYYC | | | | | | GAGTGGATGGGAAGTATCATCCCTATC | | IIPILGVANY | | | |
| | | | | | | | | | | | | | | | | CTTGGTGTCGCAAACTACGCACAGAAG | | AQKFQGRVTI | | | |
| | | | | | | | | | | | | | | | | TTCCAGGGCAGAGTCACGATTAACGCG | | NADKSTSTAY | | | |
| | | | | | | | | | | | | | | | | GACAAATCCACGAGCACAGCCTACATG | | MELSSLRSED | | | |
| | | | | | | | | | | | | | | | | GAGCTGAGCAGCCTGAGATCTGAGGAC | | TAVYYCARDL | | | |
| | | | | | | | | | | | | | | | | ACGGCCGTGTATTACTGTGCGAGAGAT | | GYSSGGANYY | | | |
| | | | | | | | | | | | | | | | | CTGGGGTATAGCAGTGGCGGGGCCAAT | | FDYWGQGTLV | | | |
| | | | | | | | | | | | | | | | | TACTACTTTGACTACTGGGGCCAGGGA | | TVSS | | | |
| | | | | | | | | | | | | | | | | ACCCTGGTCACCGTCTCCTCA | | | | | |
| ADI- | IGHV1- | QVQLVQSG | 35 | YTFTGYC | 420 | WLRQA | 515 | RINPNN | 761 | RVTMT | 1005 | ARDYSG | 1266 | RGRGTL | 1305 | CAGGTCCAGCTGGTGCAGTCTGGGGCT | 1568 | QVQLVQSGAE | 1851 | | |
| 75645 | 2 | AEVKKPGA | | IH | | PRQGL | | GGANYA | | SDTSI | | VYPMKP | | VTVSS | | GAGGTGAAGAAGCCTGGGGCCTCAGTG | | VKKPGASVKV | | | |
| | | SVKVSCKA | | | | EWMG | | QKFQG | | STAYM | | IWFDP | | | | AAGGTCTCCTGCAAGGCTTCTGGATAC | | SCKASGYTFT | | | |
| | | SG | | | | | | | | DLSRL | | | | | | ACCTTCACCGGCTACTGTATACACTGG | | GYCIHWLRQA | | | |
| | | | | | | | | | | TSDDT | | | | | | CTGCGACAGGCCCCTAGACAAGGGCTT | | PRQGLEWMGR | | | |
| | | | | | | | | | | AIYYC | | | | | | GAGTGGATGGGACGGATCAACCCCAAC | | INPNNGGANY | | | |
| | | | | | | | | | | | | | | | | AATGGTGGCGCAAACTACGCACAGAAG | | AQKFQGRVTM | | | |
| | | | | | | | | | | | | | | | | TTTCAGGGCAGGGTCACCATGACCAGC | | TSDTSISTAY | | | |
| | | | | | | | | | | | | | | | | GACACATCCATCAGCACAGCCTACATG | | MDLSRLTSDD | | | |
| | | | | | | | | | | | | | | | | GACCTGAGCAGGCTGACTTCTGACGAC | | TAIYYCARDY | | | |
| | | | | | | | | | | | | | | | | ACGGCCATATATTACTGTGCGAGAGAT | | SGVYPMKPIW | | | |
| | | | | | | | | | | | | | | | | TACTCGGGGGTGTATCCTATGAAGCCG | | FDPRGRGTLV | | | |
| | | | | | | | | | | | | | | | | ATCTGGTTCGACCCCAGGGGCCGGGGA | | TVSS | | | |
| | | | | | | | | | | | | | | | | ACCCTGGTCACCGTCTCCTCA | | | | | |
| ADI- | IGHV1- | QVQLQQSG | 188 | GDFSSYV | 421 | WVRQA | 433 | GIIPIF | 762 | RVTIT | 1006 | ARGTQD | 1267 | WGQGTL | 1286 | CAGGTACAGCTGCAGCAGTCTGGGGCT | 1569 | QVQLQQSGAE | 1852 | | |
| 75615 | 69 | AEVKKSGS | | MT | | PGQGL | | GTPNYA | | ADEST | | GATIDL | | VTVSS | | GAGGTGAAGAAGTCTGGGTCCTCGGTG | | VKKSGSSVKV | | | |
| | | SVKVSCKA | | | | EWMG | | QKFQG | | STAYM | | DY | | | | AAGGTCTCCTGCAAGGCCTCTGGAGGC | | SCKASGGDFS | | | |
| | | SG | | | | | | | | ELSSL | | | | | | GACTTCAGCAGCTATGTTATGACTTGG | | SYVMTWVRQA | | | |
| | | | | | | | | | | RSEDT | | | | | | GTGCGACAGGCCCCTGGACAGGGGCTT | | PGQGLEWMGG | | | |
| | | | | | | | | | | AVYYC | | | | | | GAGTGGATGGGAGGGATCATCCCTATC | | IIPIFGTPNY | | | |
| | | | | | | | | | | | | | | | | TTTGGGACACCAAACTACGCACAGAAA | | AQKFQGRVTI | | | |
| | | | | | | | | | | | | | | | | TTCCAGGGCAGAGTCACGATTACCGCG | | TADESTSTAY | | | |
| | | | | | | | | | | | | | | | | GACGAATCCACGAGTACAGCCTACATG | | MELSSLRSED | | | |
| | | | | | | | | | | | | | | | | GAGCTGAGCAGCCTAAGATCTGAAGAC | | TAVYYCARGT | | | |
| | | | | | | | | | | | | | | | | ACGGCCGTGTATTACTGTGCGAGAGGG | | QDGATIDLDY | | | |
| | | | | | | | | | | | | | | | | ACTCAAGATGGGGCTACAATTGATCTT | | WGQGTLVTVS | | | |
| | | | | | | | | | | | | | | | | GACTATTGGGGCCAGGGAACCCTGGTC | | S | | | |
| | | | | | | | | | | | | | | | | ACCGTCTCCTCA | | | | | |
| ADI- | IGHV4- | QVQLQQSG | 189 | GSISSYY | 263 | WIRQS | 434 | NMFYSG | 763 | RVTIS | 1007 | ARPYYD | 1268 | WGQGTT | 1287 | CAGGTGCAGCTGCAGCAGTCGGGCCCA | 1570 | QVQLQQSGPG | 1853 | | |
| 75844 | 59 | PGLVKPSE | | WS | | PGKGL | | ITNYNP | | IDTSK | | ILTGIR | | VTVSS | | GGACTGGTGAAGCCTTCGGAGACCCTG | | LVKPSETLSL | | | |
| | | TLSLICTV | | | | EWIG | | SLES | | NQLSL | | DDAFDI | | | | TCCCTCATCTGCACTGTCTCTGGCGGC | | ICTVSGGSIS | | | |
| | | SG | | | | | | | | KLSSV | | | | | | TCCATCAGTAGCTACTACTGGAGCTGG | | SYYWSWIRQS | | | |
| | | | | | | | | | | TAADT | | | | | | ATCCGGCAGTCCCCAGGGAAGGGACTG | | PGKGLEWIGN | | | |
| | | | | | | | | | | AVYYC | | | | | | GAGTGGATTGGGAATATGTTTTACAGT | | MFYSGITNYN | | | |
| | | | | | | | | | | | | | | | | GGGATCACCAACTACAACCCCTCCCTC | | PSLESRVTIS | | | |
| | | | | | | | | | | | | | | | | GAGAGTCGAGTCACCATATCAATAGAC | | IDTSKNQLSL | | | |
| | | | | | | | | | | | | | | | | ACGTCCAAGAACCAGCTCTCCCTGAAG | | KLSSVTAADT | | | |
| | | | | | | | | | | | | | | | | CTGAGCTCTGTGACCGCTGCGGACACG | | AVYYCARPYY | | | |
| | | | | | | | | | | | | | | | | GCCGTGTATTACTGTGCGAGGCCGTAT | | DILTGIRDDA | | | |
| | | | | | | | | | | | | | | | | TACGATATTTTGACTGGTATCCGAGAT | | FDIWGQGTTV | | | |
| | | | | | | | | | | | | | | | | GATGCTTTTGATATCTGGGGCCAAGGG | | TVSS | | | |
| | | | | | | | | | | | | | | | | ACCACGGTCACCGTCTCCTCA | | | | | |
| ADI- | IGHV5- | QVQLQQSG | 157 | YSFFHYW | 422 | WVRQM | 516 | TINPVN | 764 | QVTIS | 1008 | ATADRG | 1269 | WGQGTL | 1286 | CAGGTGCAGCTGCAGCAGTCCGGAGCA | 1571 | QVQLQQSGAE | 1854 | | |
| 75875 | 10-1 | AEVKKPGE | | IS | | PGKGL | | SYTNYN | | VDRSI | | | | VTVSS | | GAGGTGAAGAAACCCGGGGAGTCTCTG | | VKKPGESLRI | | | |
| | | SLRISCKG | | | | EWMA | | PSFQG | | NTVYL | | | | | | AGGATCTCCTGTAAGGGTTCTGGATAC | | SCKGSGYSFF | | | |
| | | SG | | | | | | | | QWSSL | | | | | | AGCTTTTTCCATTACTGGATCAGCTGG | | HYWISWVRQM | | | |
| | | | | | | | | | | RASDS | | | | | | GTGCGCCAGATGCCCGGGAAAGGCCTG | | PGKGLEWMAT | | | |
| | | | | | | | | | | AMYYC | | | | | | GAATGGATGGCGACCATTAATCCTGTT | | INPVNSYTNY | | | |
| | | | | | | | | | | | | | | | | AACTCTTATACCAACTACAACCCGTCC | | NPSFQGQVTI | | | |
| | | | | | | | | | | | | | | | | TTCCAAGGCCAAGTCACCATCTCAGTT | | SVDRSINTVY | | | |
| | | | | | | | | | | | | | | | | GACAGGTCCATCAACACTGTCTACCTG | | LQWSSLRASD | | | |
| | | | | | | | | | | | | | | | | CAATGGAGCAGTCTGAGGGCCTCGGAC | | SAMYYCATAD | | | |
| | | | | | | | | | | | | | | | | AGCGCCATGTATTACTGTGCGACCGCG | | RGWGQGTLVT | | | |
| | | | | | | | | | | | | | | | | GATAGGGGTTGGGGCCAGGGAACCCTG | | VSS | | | |
| | | | | | | | | | | | | | | | | GTCACCGTCTCCTCA | | | | | |
| ADI- | IGHV1- | QVPLVQSG | 190 | GTFSSYS | 356 | WVRQA | 433 | RIIPLL | 765 | RVTIT | 832 | ARENCF | 1270 | WGQGTT | 1287 | CAGGTCCCGCTGGTGCAGTCTGGGGCT | 1572 | QVPLVQSGAE | 1855 | | |
| 75714 | 69 | AEVKKPGS | | IS | | PGQGL | | GIPNYA | | ADKST | | GTSCST | | VTVSS | | GAGGTGAAGAAGCCTGGGTCCTCGGTG | | VKKPGSSVKV | | | |
| | | SVKVSCKA | | | | EWMG | | QKMQG | | STAYM | | PWGAFD | | | | AAGGTCTCCTGCAAGGCTTCTGGAGGC | | SCKASGGTFS | | | |
| | | SG | | | | | | | | ELSSL | | I | | | | ACCTTCAGCAGCTATTCTATCAGCTGG | | SYSISWVRQA | | | |
| | | | | | | | | | | RSEDT | | | | | | GTGCGACAGGCCCCTGGACAAGGACTT | | PGQGLEWMGR | | | |
| | | | | | | | | | | AVYYC | | | | | | GAGTGGATGGGAAGGATCATCCCTCTC | | IIPLLGIPNY | | | |
| | | | | | | | | | | | | | | | | CTTGGTATACCAAACTACGCACAGAAG | | AQKMQGRVTI | | | |
| | | | | | | | | | | | | | | | | ATGCAGGGCAGAGTCACGATTACCGCG | | TADKSTSTAY | | | |
| | | | | | | | | | | | | | | | | GACAAATCAACGAGCACAGCCTACATG | | MELSSLRSED | | | |
| | | | | | | | | | | | | | | | | GAGCTGAGCAGCCTGAGATCTGAGGAC | | TAVYYCAREN | | | |
| | | | | | | | | | | | | | | | | ACGGCCGTGTATTACTGTGCGAGAGAA | | CFGTSCSTPW | | | |
| | | | | | | | | | | | | | | | | AATTGTTTTGGTACCTCCTGCTCTACC | | GAFDIWGQGT | | | |
| | | | | | | | | | | | | | | | | CCGTGGGGTGCTTTTGATATCTGGGGC | | TVTVSS | | | |
| | | | | | | | | | | | | | | | | CAAGGGACCACGGTCACCGTCTCCTCA | | | | | |
| ADI- | IGHV4- | QVQLQQWG | 43 | GSFSGYY | 206 | WIRQP | 445 | EINHSG | 527 | RVTIS | 1009 | ARDSTI | 1271 | WGQGTL | 1286 | CAGGTGCAGCTGCAGCAGTGGGGCGCA | 1573 | QVQLQQWGAG | 1856 | | |
| 75688 | 34 | AGLLKPSE | | WS | | PGKGL | | STNYNP | | VDTSK | | YSSGWW | | VTVSS | | GGACTGTTGAAGCCTTCGGAGACCCTG | | LLKPSETLSL | | | |
| | | TLSLTCAV | | | | EWIG | | SLKS | | NQFSL | | SVVGSS | | | | TCCCTCACCTGCGCTGTCTATGGTGGG | | TCAVYGGSFS | | | |
| | | YG | | | | | | | | KLSSV | | LVGPAY | | | | TCCTTCAGTGGTTACTACTGGAGCTGG | | GYYWSWIRQP | | | |
| | | | | | | | | | | TAADT | | YFDY | | | | ATCCGCCAGCCCCCAGGGAAGGGGCTG | | PGKGLEWIGE | | | |
| | | | | | | | | | | AVYYC | | | | | | GAGTGGATTGGGGAAATCAATCATAGT | | INHSGSTNYN | | | |
| | | | | | | | | | | | | | | | | GGAAGCACCAACTACAACCCGTCCCTC | | PSLKSRVTIS | | | |
| | | | | | | | | | | | | | | | | AAGAGTCGAGTCACCATATCAGTAGAC | | VDTSKNQFSL | | | |
| | | | | | | | | | | | | | | | | ACGTCCAAGAACCAGTTCTCCCTGAAG | | KLSSVTAADT | | | |
| | | | | | | | | | | | | | | | | CTGAGCTCTGTGACCGCCGCGGACACG | | AVYYCARDST | | | |
| | | | | | | | | | | | | | | | | GCTGTGTATTACTGTGCGAGAGACTCG | | IYSSGWWSVV | | | |
| | | | | | | | | | | | | | | | | ACCATATATAGCAGTGGCTGGTGGTCC | | GSSLVGPAYY | | | |
| | | | | | | | | | | | | | | | | GTAGTTGGTTCGAGTTTAGTCGGGCCC | | FDYWGQGTLV | | | |
| | | | | | | | | | | | | | | | | GCTTACTACTTTGACTACTGGGGCCAG | | TVSS | | | |
| | | | | | | | | | | | | | | | | GGAACCCTGGTCACCGTCTCCTCA | | | | | |
| ADI- | HV5- | QVQLVQSG | 174 | YSFTSYW | 372 | WVRQM | 496 | IIYPGD | 635 | QVTIS | 1010 | ARGMLD | 1272 | WGHGTT | 1304 | CAGGTGCAGCTGGTGCAGTCTGGAGCA | 1574 | QVQLVQSGAE | 1857 | | |
| 75637 | 51 | AEVKKPGE | | IG | | PGKGL | | SDTRYS | | ADKSI | | TFDI | | VTVSS | | GAGGTGAAAAAGCCGGGGGAGTCTCTG | | VKKPGESLKI | | | |
| | | SLKISCKG | | | | EWLG | | PSFQG | | STAYL | | | | | | AAGATCTCCTGTAAGGGTTCTGGATAC | | SCKGSGYSFT | | | |
| | | SG | | | | | | | | QWSSL | | | | | | AGCTTTACCAGCTACTGGATCGGCTGG | | SYWIGWVRQM | | | |
| | | | | | | | | | | KASDT | | | | | | GTGCGCCAGATGCCCGGGAAAGGCCTG | | PGKGLEWLGI | | | |
| | | | | | | | | | | AMYYC | | | | | | GAGTGGCTGGGGATCATCTATCCTGGT | | IYPGDSDTRY | | | |
| | | | | | | | | | | | | | | | | GACTCTGATACCAGATACAGCCCGTCC | | SPSFQGQVTI | | | |
| | | | | | | | | | | | | | | | | TTCCAAGGCCAGGTCACCATCTCAGCC | | SADKSISTAY | | | |
| | | | | | | | | | | | | | | | | GACAAGTCCATCAGCACCGCCTACCTG | | LQWSSLKASD | | | |
| | | | | | | | | | | | | | | | | CAGTGGAGCAGCCTGAAGGCCTCGGAC | | TAMYYCARGM | | | |
| | | | | | | | | | | | | | | | | ACCGCCATGTATTACTGTGCGAGGGGA | | LDTFDIWGHG | | | |
| | | | | | | | | | | | | | | | | ATGTTAGACACTTTTGATATCTGGGGC | | TTVTVSS | | | |
| | | | | | | | | | | | | | | | | CACGGGACCACGGTCACCGTCTCCTCA | | | | | |
| ADI- | IGHV5- | QVQLVQSG | 191 | YIFTTYW | 423 | WVRQM | 476 | RIDPSD | 766 | HVTFS | 1011 | ARVFTE | 1273 | WGQGTT | 1287 | CAGGTCCAGCTTGTGCAGTCTGGAGCA | 1575 | QVQLVQSGAE | 1858 | | |
| 75745 | 10-1 | AEVKKPGE | | IT | | PGKGL | | SYTNYS | | SDKSI | | PITVAH | | VTVSS | | GAAGTGAAAAAGCCCGGGGAGTCTCTG | | VKKPGESLRI | | | |
| | | SLRISCQG | | | | EWMG | | PSFQG | | STAYL | | DAFDI | | | | AGGATCTCCTGTCAGGGTTCTGGATAC | | SCQGSGYIFT | | | |
| | | SG | | | | | | | | QWSSL | | | | | | ATCTTTACCACCTATTGGATCACTTGG | | TYWITWVRQM | | | |
| | | | | | | | | | | KASDT | | | | | | GTGCGCCAGATGCCCGGGAAAGGCCTG | | PGKGLEWMGR | | | |
| | | | | | | | | | | AIYYC | | | | | | GAGTGGATGGGACGCATTGATCCTAGT | | IDPSDSYTNY | | | |
| | | | | | | | | | | | | | | | | GACTCTTATACCAACTACAGCCCGTCC | | SPSFQGHVTF | | | |
| | | | | | | | | | | | | | | | | TTCCAAGGCCACGTCACCTTCTCATCT | | SSDKSISTAY | | | |
| | | | | | | | | | | | | | | | | GACAAGTCAATCAGCACTGCCTACCTG | | LQWSSLKASD | | | |
| | | | | | | | | | | | | | | | | CAGTGGAGCAGCCTGAAGGCCTCGGAC | | TAIYYCARVF | | | |
| | | | | | | | | | | | | | | | | ACCGCCATTTATTACTGTGCGAGAGTG | | TEPITVAHDA | | | |
| | | | | | | | | | | | | | | | | TTTACGGAGCCGATAACAGTGGCCCAT | | FDIWGQGTTV | | | |
| | | | | | | | | | | | | | | | | GATGCTTTTGATATCTGGGGCCAAGGG | | TVSS | | | |
| | | | | | | | | | | | | | | | | ACAACGGTCACCGTCTCCTCA | | | | | |
| ADI- | IGHV1- | RCPLVQSG | 192 | GTFNSYT | 424 | WVRQA | 517 | RVIPLR | 767 | RVTIT | 1012 | AREGTY | 1274 | WGKGTT | 1291 | AGGTGCCCGCTGGTGCAGTCTGGGGCT | 1576 | RCPLVQSGAE | 1859 | | |
| 75665 | 69 | AEVKKPGS | | IS | | PGQGV | | NIADYA | | ADKST | | GYEWHF | | VTVSS | | GAGGTGAAGAAGCCTGGGTCCTCGGTG | | VKKPGSSVKV | | | |
| | | SVKVSCKA | | | | EWMG | | QKFQG | | STVYM | | HYYMDV | | | | AAGGTCTCCTGCAAGGCTTCTGGAGGC | | SCKASGGTFN | | | |
| | | SG | | | | | | | | ELSSL | | | | | | ACCTTCAACAGTTACACTATCAGTTGG | | SYTISWVRQA | | | |
| | | | | | | | | | | RSEDT | | | | | | GTGCGACAGGCCCCCGGACAAGGGGTT | | PGQGVEWMGR | | | |
| | | | | | | | | | | AVYYC | | | | | | GAGTGGATGGGAAGGGTCATCCCTCTC | | VIPLRNIADY | | | |
| | | | | | | | | | | | | | | | | CGCAATATAGCAGACTACGCACAGAAG | | AQKFQGRVTI | | | |
| | | | | | | | | | | | | | | | | TTCCAGGGCAGAGTCACGATTACCGCG | | TADKSTSTVY | | | |
| | | | | | | | | | | | | | | | | GACAAATCCACGAGCACAGTCTACATG | | MELSSLRSED | | | |
| | | | | | | | | | | | | | | | | GAGCTGAGCAGCCTGAGATCTGAGGAC | | TAVYYCAREG | | | |
| | | | | | | | | | | | | | | | | ACGGCCGTGTATTATTGTGCGAGAGAG | | TYGYEWHFHY | | | |
| | | | | | | | | | | | | | | | | GGGACGTATGGTTACGAGTGGCACTTC | | YMDVWGKGTT | | | |
| | | | | | | | | | | | | | | | | CACTACTACATGGACGTCTGGGGCAAA | | VTVSS | | | |
| | | | | | | | | | | | | | | | | GGGACCACGGTCACCGTCTCCTCA | | | | | |
| ADI- | IGHV3- | RCTLQQSG | 193 | FTFNNAW | 425 | WVRQA | 431 | RIKSYV | 768 | RFTIS | 1013 | VTERRV | 1275 | WGKGTT | 1291 | AGGTGCACGCTGCAGCAGTCTGGGGGA | 1577 | RCTLQQSGGG | 1860 | | |
| 75812 | 15 | GGLVKPGE | | MS | | PGKGL | | NGGTTD | | RDDSK | | EVGATE | | VTVSS | | GGCTTGGTAAAGCCGGGGGAGTCCCTT | | LVKPGESLRL | | | |
| | | SLRLSCAA | | | | EWVA | | YVAPVK | | NTLFL | | VSYYYM | | | | AGACTCTCCTGTGCAGCCTCTGGATTC | | SCAASGFTFN | | | |
| | | SG | | | | | | G | | QMKSL | | DV | | | | ACTTTCAATAACGCCTGGATGAGTTGG | | NAWMSWVRQA | | | |
| | | | | | | | | | | KTEDT | | | | | | GTCCGCCAGGCTCCAGGGAAGGGGCTG | | PGKGLEWVAR | | | |
| | | | | | | | | | | AVYYC | | | | | | GAGTGGGTTGCCCGTATTAAAAGTTAT | | IKSYVNGGTT | | | |
| | | | | | | | | | | | | | | | | GTTAACGGTGGGACAACAGACTACGTT | | DYVAPVKGRF | | | |
| | | | | | | | | | | | | | | | | GCACCCGTGAAAGGCAGATTCACCATC | | TISRDDSKNT | | | |
| | | | | | | | | | | | | | | | | TCAAGAGATGATTCTAAAAACACTCTG | | LFLQMKSLKT | | | |
| | | | | | | | | | | | | | | | | TTTCTGCAAATGAAGAGCCTGAAAACC | | EDTAVYYCVT | | | |
| | | | | | | | | | | | | | | | | GAGGACACAGCCGTCTATTACTGTGTC | | ERRVEVGATE | | | |
| | | | | | | | | | | | | | | | | ACTGAGCGACGTGTAGAAGTGGGAGCT | | VSYYYMDVWG | | | |
| | | | | | | | | | | | | | | | | ACTGAGGTCTCCTACTACTACATGGAC | | KGTTVTVSS | | | |
| | | | | | | | | | | | | | | | | GTCTGGGGCAAAGGGACCACGGTCACC | | | | | |
| | | | | | | | | | | | | | | | | GTCTCCTCA | | | | | |
| ADI- | IGHV3- | QVQLVQSG | 13 | FTFSSYA | 199 | WVRQA | 518 | VINSDG | 769 | RFTIS | 1014 | ARESMD | 1276 | WGRGTL | 1288 | CAGGTGCAGCTGGTGCAGTCTGGGGGA | 1578 | QVQLVQSGGG | 1861 | | |
| 75788 | 64 | GGLVKPGG | | MH | | PGKGL | | GSPYYA | | RDNSK | | QTAYFD | | VTVSS | | GGCTTGGTCAAACCTGGGGGGTCCCTG | | LVKPGGSLRL | | | |
| | | SLRLSCAA | | | | EFVS | | NAVKG | | NKVFL | | Y | | | | AGACTCTCCTGTGCAGCCTCTGGGTTC | | SCAASGFTFS | | | |
| | | SG | | | | | | | | QMGSL | | | | | | ACCTTCAGTTCCTATGCTATGCACTGG | | SYAMHWVRQA | | | |
| | | | | | | | | | | RAEDM | | | | | | GTCCGCCAGGCTCCAGGGAAGGGACTG | | PGKGLEFVSV | | | |
| | | | | | | | | | | AVYYC | | | | | | GAATTTGTTTCAGTTATTAATAGTGAT | | INSDGGSPYY | | | |
| | | | | | | | | | | | | | | | | GGGGGTAGCCCATATTATGCAAACGCT | | ANAVKGRFTI | | | |
| | | | | | | | | | | | | | | | | GTGAAGGGCAGATTCACCATCTCCAGA | | SRDNSKNKVF | | | |
| | | | | | | | | | | | | | | | | GACAATTCCAAGAACAAGGTGTTTCTG | | LQMGSLRAED | | | |
| | | | | | | | | | | | | | | | | CAAATGGGCAGCCTGAGAGCTGAGGAC | | MAVYYCARES | | | |
| | | | | | | | | | | | | | | | | ATGGCTGTATATTACTGTGCGAGAGAG | | MDQTAYFDYW | | | |
| | | | | | | | | | | | | | | | | TCTATGGACCAGACGGCTTACTTTGAC | | GRGTLVTVSS | | | |
| | | | | | | | | | | | | | | | | TACTGGGGCCGGGGAACCCTGGTCACC | | | | | |
| | | | | | | | | | | | | | | | | GTCTCCTCA | | | | | |
| ADI- | IGHV3- | RSSCMQSG | 194 | FTFSRFA | 248 | WVRQA | 431 | VVSFDG | 770 | RFTIS | 779 | ARGAYY | 1277 | WGQGAL | 1285 | AGGTCCAGCTGCATGCAGTCTGGGGAG | 1579 | RSSCMQSGEA | 1862 | | |
| 75599 | 30 | EAWSSWGV | | MH | | PGKGL | | SNKYYA | | RDNSK | | GSGSYY | | VTVSS | | GCGTGGTCCAGCTGGGGGGTCCCTGAG | | WSSWGVPETL | | | |
| | | PETL- | | | | EWVA | | DSMKG | | NTVYL | | NPLPTF | | | | ACTCTCTGTGCAGCCTCTAACTTCACT | | CAASNFTFSR | | | |
| | | CAASN | | | | | | | | QMNSL | | DY | | | | TTCAGTAGGTTTGCTATGCACTGGGTC | | FAMHWVRQAP | | | |
| | | | | | | | | | | RAEDT | | | | | | CGCCAGGCTCCAGGCAAGGGGCTGGAG | | GKGLEWVAVV | | | |
| | | | | | | | | | | AVYYC | | | | | | TGGGTGGCAGTTGTATCATTTGATGGA | | SFDGSNKYYA | | | |
| | | | | | | | | | | | | | | | | AGTAATAAATACTACGCAGACTCCATG | | DSMKGRFTIS | | | |
| | | | | | | | | | | | | | | | | AAGGGCCGCTTCACCATCTCCAGAGAC | | RDNSKNTVYL | | | |
| | | | | | | | | | | | | | | | | AATTCCAAGAACACGGTGTATCTGCAA | | QMNSLRAEDT | | | |
| | | | | | | | | | | | | | | | | ATGAACAGCCTGAGAGCTGAGGACACG | | AVYYCARGAY | | | |
| | | | | | | | | | | | | | | | | GCTGTGTATTACTGTGCGAGAGGAGCT | | YGSGSYYNPL | | | |
| | | | | | | | | | | | | | | | | TACTATGGTTCAGGGAGTTATTATAAC | | PTFDYWGQGA | | | |
| | | | | | | | | | | | | | | | | CCACTGCCCACGTTTGACTACTGGGGC | | LVTVSS | | | |
| | | | | | | | | | | | | | | | | CAGGGAGCCCTGGTCACCGTCTCCTCA | | | | | |
| ADI- | IGHV5- | QVQLVQSG | 174 | YSFTSYW | 372 | WVRQM | 476 | IIYPGD | 635 | QVTIS | 1010 | ARTMLD | 1278 | WGQGTT | 1287 | CAGGTCCAGCTGGTGCAGTCTGGAGCA | 1580 | QVQLVQSGAE | 1863 | | |
| 75664 | 51 | AEVKKPGE | | IG | | PGKGL | | SDTRYS | | ADKSI | | DAFDI | | VTVSS | | GAGGTGAAAAAGCCCGGGGAGTCTCTG | | VKKPGESLKI | | | |
| | | SLKISCKG | | | | EWMG | | PSFQG | | STAYL | | | | | | AAGATCTCCTGTAAGGGTTCTGGATAC | | SCKGSGYSFT | | | |
| | | SG | | | | | | | | QWSSL | | | | | | AGCTTTACCAGCTACTGGATCGGCTGG | | SYWIGWVRQM | | | |
| | | | | | | | | | | KASDT | | | | | | GTGCGCCAGATGCCCGGGAAAGGCCTG | | PGKGLEWMGI | | | |
| | | | | | | | | | | AMYYC | | | | | | GAGTGGATGGGGATCATCTATCCTGGT | | IYPGDSDTRY | | | |
| | | | | | | | | | | | | | | | | GACTCTGATACCAGATACAGCCCGTCC | | SPSFQGQVTI | | | |
| | | | | | | | | | | | | | | | | TTCCAAGGCCAGGTCACCATCTCAGCC | | SADKSISTAY | | | |
| | | | | | | | | | | | | | | | | GACAAGTCCATCAGCACCGCCTACCTG | | LQWSSLKASD | | | |
| | | | | | | | | | | | | | | | | CAGTGGAGCAGCCTGAAGGCCTCGGAC | | TAMYYCARTM | | | |
| | | | | | | | | | | | | | | | | ACCGCCATGTATTACTGTGCGAGGACT | | LDDAFDIWGQ | | | |
| | | | | | | | | | | | | | | | | ATGCTTGATGATGCTTTTGATATCTGG | | GTTVTVSS | | | |
| | | | | | | | | | | | | | | | | GGCCAAGGGACAACGGTCACCGTCTCC | | | | | |
| | | | | | | | | | | | | | | | | TCA | | | | | |
| ADI- | IGHV4- | QVQLQQSG | 195 | DSISSYY | 320 | WIRQP | 445 | YIYYSG | 771 | RVTIS | 1009 | ARGENY | 1279 | WGQGTL | 1286 | CAGGTGCAGCTGCAGCAGTCGGGCCCA | 1581 | QVQLQQSGPG | 1864 | | |
| 75646 | 59 | PGLVKPSE | | WS | | PGKGL | | STNYNP | | VDTSK | | GDYFDF | | VTVSS | | GGACTGGTGAAGCCTTCGGAGACCCTG | | LVKPSETLSL | | | |
| | | TLSLTCTV | | | | EWIG | | SLKS | | NQFSL | | | | | | TCCCTCACCTGCACTGTCTCTGGTGAC | | TCTVSGDSIS | | | |
| | | SG | | | | | | | | KLSSV | | | | | | TCCATCAGTAGTTACTACTGGAGCTGG | | SYYWSWIRQP | | | |
| | | | | | | | | | | TAADT | | | | | | ATCCGGCAGCCCCCAGGGAAGGGACTG | | PGKGLEWIGY | | | |
| | | | | | | | | | | AVYYC | | | | | | GAGTGGATTGGGTATATCTATTACAGT | | IYYSGSTNYN | | | |
| | | | | | | | | | | | | | | | | GGGAGCACCAACTACAACCCCTCCCTC | | PSLKSRVTIS | | | |
| | | | | | | | | | | | | | | | | AAGAGTCGAGTCACCATATCAGTAGAC | | VDTSKNQFSL | | | |
| | | | | | | | | | | | | | | | | ACGTCCAAGAACCAGTTCTCCCTGAAG | | KLSSVTAADT | | | |
| | | | | | | | | | | | | | | | | CTGAGCTCTGTGACCGCTGCGGACACG | | AVYYCARGEN | | | |
| | | | | | | | | | | | | | | | | GCCGTGTATTACTGTGCGAGAGGGGAA | | YGDYFDFWGQ | | | |
| | | | | | | | | | | | | | | | | AACTACGGTGACTACTTTGACTTCTGG | | GTLVTVSS | | | |
| | | | | | | | | | | | | | | | | GGCCAGGGAACCCTGGTCACCGTCTCC | | | | | |
| | | | | | | | | | | | | | | | | TCA | | | | | |
| ADI- | IGHV3- | QVQLVQSG | 13 | FTFNIAW | 426 | WVRQA | 432 | RIQSKN | 772 | RFTIS | 1015 | VGPGYC | 1280 | WGQGTL | 1286 | CAGGTGCAGCTGGTGCAGTCTGGGGGA | 1582 | QVQLVQSGGG | 1865 | | |
| 75723 | 15 | GGLVKPGG | | MS | | PGKGL | | VGGTTD | | RDDSK | | SSTSCY | | VTVSS | | GGCTTGGTAAAGCCTGGGGGGTCCCTT | | LVKPGGSLRL | | | |
| | | SLRLSCAA | | | | EWVG | | YAAPVK | | KMLYL | | ASHHDI | | | | AGACTCTCCTGTGCAGCCTCTGGATTC | | SCAASGFTFN | | | |
| | | SG | | | | | | G | | QMNSL | | LTGYYL | | | | ACTTTCAATATCGCCTGGATGAGCTGG | | IAWMSWVRQA | | | |
| | | | | | | | | | | KTEDT | | PDY | | | | GTCCGCCAGGCTCCAGGGAAGGGGCTG | | PGKGLEWVGR | | | |
| | | | | | | | | | | AVYFC | | | | | | GAGTGGGTTGGTCGTATTCAAAGTAAA | | IQSKNVGGTT | | | |
| | | | | | | | | | | | | | | | | AATGTTGGTGGGACAACAGATTACGCT | | DYAAPVKGRF | | | |
| | | | | | | | | | | | | | | | | GCACCCGTGAAAGGCAGATTCACCATC | | TISRDDSKKM | | | |
| | | | | | | | | | | | | | | | | TCAAGAGATGATTCAAAAAAAATGCTG | | LYLQMNSLKT | | | |
| | | | | | | | | | | | | | | | | TATCTGCAAATGAACAGCCTGAAAACC | | EDTAVYFCVG | | | |
| | | | | | | | | | | | | | | | | GAGGACACAGCCGTGTATTTTTGTGTT | | PGYCSSTSCY | | | |
| | | | | | | | | | | | | | | | | GGACCCGGATATTGTAGTAGTACCAGC | | ASHHDILTGY | | | |
| | | | | | | | | | | | | | | | | TGCTATGCGAGTCATCACGATATTTTG | | YLPDYWGQGT | | | |
| | | | | | | | | | | | | | | | | ACTGGTTATTACCTCCCTGACTACTGG | | LVTVSS | | | |
| | | | | | | | | | | | | | | | | GGCCAGGGAACCCTGGTCACCGTCTCC | | | | | |
| | | | | | | | | | | | | | | | | TCA | | | | | |
| ADI- | IGHV3- | QITLKESG | 196 | FTFRTFA | 427 | WVRQA | 431 | VISFDG | 773 | RFTIS | 1016 | ARGDYY | 1281 | WGQGSL | 1289 | CAGATCACGTTGAAGGAGTCTGGGGGA | 1583 | QITLKESGGG | 1866 | | |
| 75595 | 30 | GGVVQPGR | | MH | | PGKGL | | NNKYYV | | RDNSK | | GSGKYF | | VTVSS | | GGCGTGGTCCAGCCTGGGAGGTCCCTG | | VVQPGRSLRL | | | |
| | | SLRLSCVA | | | | EWVA | | DSVKG | | NTVYL | | NPLPTF | | | | AGACTCTCCTGTGTAGCCTCTAGATTC | | SCVASRFTFR | | | |
| | | SR | | | | | | | | QMNSL | | DY | | | | ACCTTCAGGACTTTTGCTATGCACTGG | | TFAMHWVRQA | | | |
| | | | | | | | | | | RSEDT | | | | | | GTCCGCCAGGCTCCGGGCAAGGGGCTG | | PGKGLEWVAV | | | |
| | | | | | | | | | | AVYYC | | | | | | GAGTGGGTGGCAGTTATATCATTTGAT | | ISFDGNNKYY | | | |
| | | | | | | | | | | | | | | | | GGAAATAATAAATATTATGTAGACTCC | | VDSVKGRFTI | | | |
| | | | | | | | | | | | | | | | | GTGAAGGGCCGATTCACCATCTCCAGA | | SRDNSKNTVY | | | |
| | | | | | | | | | | | | | | | | GACAATTCCAAGAACACGGTGTATCTG | | LQMNSLRSED | | | |
| | | | | | | | | | | | | | | | | CAAATGAACAGCCTGAGAAGTGAGGAC | | TAVYYCARGD | | | |
| | | | | | | | | | | | | | | | | ACGGCTGTGTATTACTGTGCGAGAGGA | | YYGSGKYFNP | | | |
| | | | | | | | | | | | | | | | | GATTACTATGGTTCAGGGAAATATTTT | | LPTFDYWGQG | | | |
| | | | | | | | | | | | | | | | | AATCCACTGCCCACCTTTGACTACTGG | | SLVTVSS | | | |
| | | | | | | | | | | | | | | | | GGCCAGGGATCCCTGGTCACCGTCTCC | | | | | |
| | | | | | | | | | | | | | | | | TCA | | | | | |
| ADI- | IGHV3- | QVPLKQSG | 197 | SDFSTYG | 428 | WVRQA | 431 | ALSHDG | 774 | RFTIS | 1017 | AKDLIT | 1282 | WGQGTL | 1286 | CAGGTCCCGCTGAAGCAGTCTGGGGGA | 1584 | QVPLKQSGGG | 1867 | | |
| 75824 | 30 | GGVVQPGR | | MH | | PGKGL | | GYKYYA | | RDNSK | | YDFWSG | | VTVSS | | GGCGTGGTCCAGCCTGGGAGGTCCCAG | | VVQPGRSQRV | | | |
| | | SQRVSCTV | | | | EWVA | | DSVKG | | KTVYL | | FFT | | | | AGAGTCTCCTGCACAGTCTCTGGATCC | | SCTVSGSDFS | | | |
| | | SG | | | | | | | | QMNSV | | | | | | GACTTCAGTACTTACGGCATGCACTGG | | TYGMHWVRQA | | | |
| | | | | | | | | | | RPEDT | | | | | | GTCCGCCAGGCTCCAGGCAAGGGGCTG | | PGKGLEWVAA | | | |
| | | | | | | | | | | AVYYC | | | | | | GAGTGGGTGGCAGCTCTATCACATGAT | | LSHDGGYKYY | | | |
| | | | | | | | | | | | | | | | | GGAGGTTATAAATACTATGCAGACTCA | | ADSVKGRFTI | | | |
| | | | | | | | | | | | | | | | | GTGAAGGGCCGATTCACCATCTCCAGA | | SRDNSKKTVY | | | |
| | | | | | | | | | | | | | | | | GACAATTCCAAGAAAACGGTGTATCTG | | LQMNSVRPED | | | |
| | | | | | | | | | | | | | | | | CAAATGAACAGTGTGAGACCTGAGGAC | | TAVYYCAKDL | | | |
| | | | | | | | | | | | | | | | | ACGGCTGTCTATTACTGTGCGAAGGAC | | ITYDFWSGFF | | | |
| | | | | | | | | | | | | | | | | CTGATTACGTACGATTTCTGGAGTGGC | | TWGQGTLVTV | | | |
| | | | | | | | | | | | | | | | | TTTTTCACTTGGGGCCAGGGAACCCTG | | SS | | | |
| | | | | | | | | | | | | | | | | GTCACCGTCTCCTCA | | | | | |
| ADI- | IGHV5- | QVQLVQSG | 174 | YSFTSYW | 372 | WVRQM | 476 | IIYPGD | 775 | QVTIS | 1010 | ASSYCS | 1283 | WGQGTL | 1286 | CAGGTCCAGCTTGTGCAGTCTGGAGCA | 1585 | QVQLVQSGAE | 1868 | | |
| 75749 | 51 | AEVKKPGE | | IG | | PGKGL | | SDIRYS | | ADKSI | | STSCYA | | VTVSS | | GAGGTGAAAAAGCCCGGGGAGTCTCTG | | VKKPGESLKI | | | |
| | | SLKISCKG | | | | EWMG | | PSFQG | | STAYL | | AKY | | | | AAGATCTCCTGTAAGGGTTCTGGATAC | | SCKGSGYSFT | | | |
| | | SG | | | | | | | | QWSSL | | | | | | AGCTTTACCAGCTACTGGATCGGCTGG | | SYWIGWVRQM | | | |
| | | | | | | | | | | KASDT | | | | | | GTGCGCCAGATGCCCGGGAAAGGCCTG | | PGKGLEWMGI | | | |
| | | | | | | | | | | AMYYC | | | | | | GAGTGGATGGGGATCATCTATCCTGGT | | IYPGDSDIRY | | | |
| | | | | | | | | | | | | | | | | GACTCTGATATCAGATACAGCCCGTCC | | SPSFQGQVTI | | | |
| | | | | | | | | | | | | | | | | TTCCAAGGCCAGGTCACCATCTCAGCC | | SADKSISTAY | | | |
| | | | | | | | | | | | | | | | | GACAAGTCCATCAGCACCGCCTACCTG | | LQWSSLKASD | | | |
| | | | | | | | | | | | | | | | | CAGTGGAGCAGCCTGAAGGCCTCGGAC | | TAMYYCASSY | | | |
| | | | | | | | | | | | | | | | | ACCGCCATGTATTACTGTGCGAGCTCA | | CSSTSCYAAK | | | |
| | | | | | | | | | | | | | | | | TATTGTAGTAGTACCAGCTGCTATGCT | | YWGQGTLVTV | | | |
| | | | | | | | | | | | | | | | | GCCAAGTACTGGGGCCAGGGAACCCTG | | SS | | | |
| | | | | | | | | | | | | | | | | GTCACCGTCTCCTCA | | | | | |
| ADI- | IGHV1- | QVQLVQSG | 50 | GPFSTYA | 429 | WVRQA | 433 | EIIPIS | 776 | RVTIS | 1018 | ARVGRY | 1284 | WGQGTL | 1286 | CAGGTGCAGCTGGTGCAGTCTGGGGCT | 1586 | QVQLVQSGAE | 1869 | | |
| 75867 | 69 | AEVKKPGS | | IS | | PGQGL | | GTANYA | | ADEST | | YYDTSG | | VTVSS | | GAGGTGAAGAAGCCTGGGTCCTCGGTG | | VKKPGSSVKV | | | |
| | | SVKVSCKA | | | | EWMG | | QKFQG | | STAYM | | YYYTYF | | | | AAGGTCTCCTGCAAGGCTTCTGGAGGC | | SCKASGGPFS | | | |
| | | SG | | | | | | | | ELSSL | | DY | | | | CCCTTCAGCACCTATGCTATCAGCTGG | | TYAISWVRQA | | | |
| | | | | | | | | | | RSEDT | | | | | | GTGCGACAGGCCCCTGGACAAGGGCTT | | PGQGLEWMGE | | | |
| | | | | | | | | | | AVYYC | | | | | | GAGTGGATGGGAGAGATCATCCCTATC | | IIPISGTANY | | | |
| | | | | | | | | | | | | | | | | TCTGGTACAGCAAACTACGCACAGAAG | | AQKFQGRVTI | | | |
| | | | | | | | | | | | | | | | | TTCCAGGGCAGAGTCACGATTAGCGCG | | SADESTSTAY | | | |
| | | | | | | | | | | | | | | | | GACGAATCTACGAGCACAGCCTACATG | | MELSSLRSED | | | |
| | | | | | | | | | | | | | | | | GAGCTGAGCAGCCTGAGATCTGAGGAC | | TAVYYCARVG | | | |
| | | | | | | | | | | | | | | | | ACGGCCGTGTATTATTGTGCGAGAGTC | | RYYYDTSGYY | | | |
| | | | | | | | | | | | | | | | | GGTCGCTATTACTATGATACTAGTGGT | | YTYFDYWGQG | | | |
| | | | | | | | | | | | | | | | | TATTACTACACATACTTTGACTACTGG | | TLVTVSS | | | |
| | | | | | | | | | | | | | | | | GGCCAGGGAACCCTGGTCACCGTCTCC | | | | | |
| | | | | | | | | | | | | | | | | TTCA | | | | | |
| VYD224 | IGHV4- | QVQLQQWG | 93 | GAFSGYF | 295 | WIRQS | 471 | EVTHSG | 624 | RVTMS | 1019 | ARGDFY | 1124 | WGQGTL | 1286 | CAGGTGCAGCTACAACAGTGGGGCGCA | 1587 | QVQLQQWGAG | 1870 | QVQLQQWG | 1870 |
| (ADI- | 34 | AGLLKPSE | | WN | | PVKGL | | NTNYNP | | VDASK | | STRLAF | | VTVSS | | GGACTGCTGAAGCCTTCGGAGACCCTG | | LLKPSETLSL | | AGLLKPSE | |
| 80707) | | TLSLTCGV | | | | EWIG | | SLKS | | NEFSL | | DV | | | | TCCCTCACCTGCGGTGTCCACGATGGG | | TCGVHDGAFS | | TLSLTCGV | |
| | | HD | | | | | | | | QLTSV | | | | | | GCCTTCAGTGGTTACTTCTGGAACTGG | | GYFWNWIRQS | | HDGAFSGY | |
| | | | | | | | | | | TAADT | | | | | | ATCCGCCAGTCCCCAGTGAAGGGGCTG | | PVKGLEWIGE | | FWNWIRQS | |
| | | | | | | | | | | AVYYC | | | | | | GAGTGGATTGGGGAAGTCACTCATAGT | | VTHSGNTNYN | | PVKGLEWI | |
| | | | | | | | | | | | | | | | | GGCAACACCAACTACAACCCGTCCCTC | | PSLKSRVTMS | | GEVTHSGN | |
| | | | | | | | | | | | | | | | | AAGAGTCGAGTCACCATGTCAGTAGAC | | VDASKNEFSL | | TNYNPSLK | |
| | | | | | | | | | | | | | | | | GCGTCCAAGAACGAATTCTCCCTGCAA | | QLTSVTAADT | | SRVTMSVD | |
| | | | | | | | | | | | | | | | | CTGACCTCTGTGACCGCCGCGGACACG | | AVYYCARGDF | | ASKNEFSL | |
| | | | | | | | | | | | | | | | | GCTGTGTATTACTGTGCGAGGGGCGAC | | YSTRLAFDVW | | QLTSVTAA | |
| | | | | | | | | | | | | | | | | TTTTATAGCACCAGGCTCGCTTTTGAT | | GQGTLVTVSS | | DTAVYYCA | |
| | | | | | | | | | | | | | | | | GTCTGGGGCCAAGGGACACTGGTCACC | | | | RGDFYSTR | |
| | | | | | | | | | | | | | | | | GTCTCTTCA | | | | LAFDVWGQ | |
| | | | | | | | | | | | | | | | | | | | | GTLVTVSS | |
| VYD225 | | QVQLQESG | 21 | ASISQHD | 430 | WVRQS | 438 | AIFHSG | 538 | RLTIS | 1020 | ARARGI | 1039 | WGQGTL | 1286 | caggtccaactgcaagaatctggcccc | 1588 | QVQLQESGPG | 1871 | | |
| | | PGLVKPSG | | WWT | | PGKGL | | TTNYNP | | VDTSK | | LDF | | VTVSS | | gggctcgtcaagccttctggaactttg | | LVKPSGTLSL | | | |
| | | TLSLTCAV | | | | EWIG | | SLRS | | SHFSL | | | | | | tccctgacatgcgccgtgtctggcgcg | | TCAVSGASIS | | | |
| | | SG | | | | | | | | KLTSV | | | | | | tcaatatcacagcatgactggtggacc | | QHDWWTWVRQ | | | |
| | | | | | | | | | | TAADT | | | | | | tgggttaggcagagccccggcaagggg | | SPGKGLEWIG | | | |
| | | | | | | | | | | AVYYC | | | | | | ctcgagtggattggagccatatttcat | | AIFHSGTTNY | | | |
| | | | | | | | | | | | | | | | | tctggcacaaccaactacaacccttct | | NPSLRSRLTI | | | |
| | | | | | | | | | | | | | | | | ttgcggagccggttgaccattagcgtg | | SVDTSKSHFS | | | |
| | | | | | | | | | | | | | | | | gacacctccaaatcacatttttcgctg | | LKLTSVTAAD | | | |
| | | | | | | | | | | | | | | | | aagctcacctccgtcactgccgctgac | | TAVYYCARAR | | | |
| | | | | | | | | | | | | | | | | accgctgtgtattactgtgcacgggct | | GILDFWGQGT | | | |
| | | | | | | | | | | | | | | | | cgtggaatcctggatttctggggccag | | LVTVSS | | | |
| | | | | | | | | | | | | | | | | gggacacttgtcacggtatccagc | | | | | |

TABLE 4

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75846 | IGKV1-33 | DIQLTQ | 1876 | QASQDIT | 2048 | WYQQKP | 2257 | DAVNLE | 2382 | GVPSRFSG | 2528 | QQYDN | 2700 | FGGGTK | 2936 |
| | | SPSSLS | | NYLS | | GKAPKL | | T | | SGSGTDFT | | LPLT | | VEIK | |
| | | ASVGDR | | | | LIY | | | | FTISSLQP | | | | | |
| | | VTITC | | | | | | | | EDFATYYC | | | | | |
| ADI-75829 | IGKV3D- | EIAMTQ | 1877 | RASQSVN | 2049 | WYQQKP | 2258 | GASTRA | 2383 | GIPARFSG | 2529 | QQYNI | 2701 | FGQGTK | 2937 |
| | 15 | SPATLS | | SNLA | | GQAPRL | | T | | SGSGTEFT | | WPLYT | | VEIK | |
| | | VSPGER | | | | LIY | | | | LTISSLQS | | | | | |
| | | ATLSC | | | | | | | | EDFAVYFC | | | | | |
| ADI-75811 | IGKV1D- | DIQMTQ | 1878 | RASQSIS | 2050 | WYQQKP | 2257 | SASSLQ | 2384 | GVPSRFSG | 2530 | QQSYN | 2702 | FGQGTK | 2937 |
| | 39 | SPSSLS | | SYLN | | GKAPKL | | S | | SGSGTDFS | | TPPWT | | VEIK | |
| | | ASVGDR | | | | LIY | | | | LTISSLQP | | | | | |
| | | VTITC | | | | | | | | EDFATYYC | | | | | |
| ADI-75585 | IGKV1- | DIQLTQ | 1876 | RASQAIS | 2051 | WYQQKP | 2259 | AASILE | 2385 | GVPSRFSG | 2531 | QQYYS | 2703 | FGGGTK | 2938 |
| | NL1 | SPSSLS | | NSLA | | GKAPKL | | S | | SGSGTDYT | | SLGRT | | VDIK | |
| | | ASVGDR | | | | LLY | | | | LTISSLQP | | | | | |
| | | VTITC | | | | | | | | EDFATYYC | | | | | |
| ADI-75869 | IGKV1-5 | DIQMTQ | 1879 | RASQSIS | 2052 | WYQQKP | 2257 | KASSLE | 2386 | GVPSRFSG | 2532 | QQYDS | 2704 | FGQGTK | 2937 |
| | | SPSTLS | | RWLA | | GKAPKL | | S | | SGSGTEFT | | YPWT | | VEIK | |
| | | ASVGDR | | | | LIY | | | | LTISSLQP | | | | | |
| | | VTITC | | | | | | | | DDFATYYC | | | | | |
| ADI-75817 | IGKV2D- | DIQMTQ | 1880 | RSSQSLL | 2053 | WYLQKP | 2260 | LGSNRA | 2387 | GVPDRFSG | 2533 | MQGLQ | 2705 | FGGGTK | 2936 |
| | 28 | SPNSLP | | HTNGYNY | | GQSPQL | | S | | SGSGTDFT | | TPPT | | VEIK | |
| | | VTPGEP | | LD | | LIY | | | | LKISRVEA | | | | | |
| | | ASISC | | | | | | | | EDVGVYYC | | | | | |
| ADI-75590 | IGKV1D- | EIQMTQ | 1881 | RASQGIG | 2054 | WYRQRP | 2261 | DASSLE | 2388 | GVPSRFSG | 2534 | QQFNN | 2706 | FGGGTK | 2936 |
| | 13 | SPSSLS | | SALA | | GKPPKL | | G | | SGSGTDFT | | RIT | | VEIK | |
| | | ASVGDR | | | | LIS | | | | LTISSLQP | | | | | |
| | | VTITC | | | | | | | | EDFATYYC | | | | | |
| ADI-75823 | IGKV1D- | DITMTQ | 1882 | RASQSIR | 2055 | WYQQKP | 2257 | DASSLQ | 2389 | GVPSRFSG | 2534 | QQSYN | 2702 | FGQGTK | 2937 |
| | 39 | SPSSLS | | RFLN | | GKAPKL | | S | | SGSGTDFT | | TPPWT | | VEIK | |
| | | ASVGDR | | | | LIY | | | | LTISSLQP | | | | | |
| | | VTITC | | | | | | | | EDFATYYC | | | | | |
| ADI-75569 | IGLV3-21 | SYVLTQ | 1883 | EGNYIGG | 2056 | WYQQRP | 2262 | DDFGRP | 2390 | GIPERFSG | 2535 | QVWDT | 2707 | FGGGTK | 2939 |
| | | PPSVSV | | QSVN | | GQAPVV | | S | | SNSGNTAT | | SSDRI | | LTVL | |
| | | APGQTA | | | | VLY | | | | LTINWVEA | | L | | | |
| | | RITC | | | | | | | | GDEADYYC | | | | | |
| ADI-75623 | IGKV1D- | DMTMTQ | 1884 | RASQSIS | 2057 | WYQQKP | 2263 | AASSLQ | 2391 | GVPSRFSG | 2536 | QQSYT | 2708 | FGQGTR | 2940 |
| | 39 | SPSSLS | | KFLN | | GKAPNL | | S | | SGSGTDFT | | MPPVT | | LEIK | |
| | | ASVGDR | | | | LIY | | | | LTINSLQP | | | | | |
| | | VTITC | | | | | | | | EDFATYYC | | | | | |
| ADI-75626 | IGKV1D- | DIQMTQ | 1878 | RASQSIS | 2058 | WFQQRP | 2264 | AASTLQ | 2392 | GVPSRFSG | 2534 | QQSFV | 2709 | FGRGTK | 2941 |
| | 39 | SPSSLS | | DFLS | | GKAPKL | | S | | SGSGTDFT | | TPYT | | VEIK | |
| | | ASVGDR | | | | LIY | | | | LTISSLQP | | | | | |
| | | VTITC | | | | | | | | EDFATYYC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75846 | GACATCCAGTTGACCCAGTCTCCATCCTCCCTGTCTGCATCTGTAGGAGACAGAGTCACCA | 2962 | DIQLTQSPSSLSASVGDRVTITCQASQD | 3245 | | |
| | TCACTTGCCAGGCGAGTCAGGACATTACCAACTATTTATCTTGGTATCAACAGAAACCAGG | | ITNYLSWYQQKPGKAPKLLIYDAVNLET | | | |
| | GAAAGCCCCTAAGCTCCTGATCTACGATGCAGTCAATTTGGAAACAGGGGTCCCATCAAGG | | GVPSRFSGSGSGTDFTFTISSLQPEDFA | | | |
| | TTCAGTGGCAGTGGATCTGGGACAGATTTTACTTTCACCATCAGCAGCCTGCAGCCTGAAG | | TYYCQQYDNLPLTFGGGTKVEIK | | | |
| | ATTTTGCAACATATTATTGTCAACAATATGATAATCTCCCGCTCACTTTCGGCGGAGGGAC | | | | | |
| | CAAGGTGGAAATCAAA | | | | | |
| ADI-75829 | GAAATTGCAATGACGCAGTCTCCAGCCACCCTGTCTGTGTCTCCAGGGGAAAGAGCCACCC | 2963 | EIAMTQSPATLSVSPGERATLSCRASQS | 3246 | | |
| | TCTCCTGCAGGGCCAGCCAGAGTGTTAACAGCAACTTAGCCTGGTACCAGCAGAAACCTGG | | VNSNLAWYQQKPGQAPRLLIYGASTRAT | | | |
| | CCAGGCTCCCAGGCTCCTCATCTATGGTGCATCCACCAGGGCCACTGGTATCCCAGCCAGG | | GIPARFSGSGSGTEFTLTISSLQSEDFA | | | |
| | TTCAGTGGCAGTGGGTCTGGGACAGAGTTCACTCTCACCATCAGCAGCCTGCAGTCTGAAG | | VYFCQQYNIWPLYTFGQGTKVEIK | | | |
| | ATTTTGCAGTTTATTTCTGTCAGCAGTATAATATCTGGCCTCTATACACTTTTGGCCAGGG | | | | | |
| | GACCAAGGTGGAAATCAAA | | | | | |
| ADI-75811 | GACATCCAGATGACCCAGTCTCCATCCTCCCTGTCTGCATCTGTAGGAGACAGAGTCACCA | 2964 | DIQMTQSPSSLSASVGDRVTITCRASQS | 3247 | | |
| | TCACTTGCCGGGCAAGTCAGAGCATAAGCAGCTATTTAAATTGGTATCAGCAGAAACCAGG | | ISSYLNWYQQKPGKAPKLLIYSASSLQS | | | |
| | GAAAGCCCCTAAGCTCCTGATCTATTCTGCATCCAGTTTGCAAAGTGGGGTCCCATCAAGG | | GVPSRFSGSGSGTDFSLTISSLQPEDFA | | | |
| | TTCAGTGGCAGTGGATCTGGGACAGATTTCAGTCTCACCATCAGCAGTCTGCAACCTGAAG | | TYYCQQSYNTPPWTFGQGTKVEIK | | | |
| | ATTTTGCAACTTACTACTGTCAACAGAGTTACAATACCCCTCCGTGGACGTTCGGCCAAGG | | | | | |
| | GACCAAGGTGGAGATCAAA | | | | | |
| ADI-75585 | GACATCCAGTTGACCCAGTCTCCATCCTCCCTGTCTGCATCTGTAGGAGACAGAGTCACCA | 2965 | DIQLTQSPSSLSASVGDRVTITCRASQA | 3248 | | |
| | TCACTTGCCGGGCGAGTCAGGCCATTAGCAATTCTTTAGCCTGGTATCAGCAGAAACCAGG | | ISNSLAWYQQKPGKAPKLLLYAASILES | | | |
| | GAAAGCCCCTAAGCTCCTGCTCTATGCTGCATCCATATTGGAGAGTGGGGTCCCATCCAGG | | GVPSRFSGSGSGTDYTLTISSLQPEDFA | | | |
| | TTCAGTGGCAGTGGATCTGGGACGGATTACACTCTCACTATCAGCAGCCTGCAGCCTGAAG | | TYYCQQYYSSLGRTFGGGTKVDIK | | | |
| | ATTTTGCAACTTATTACTGTCAACAGTATTATAGTAGTCTCGGGCGCACTTTCGGCGGAGG | | | | | |
| | GACCAAAGTGGATATCAAA | | | | | |
| ADI-75869 | GACATCCAGATGACCCAGTCTCCTTCCACCCTGTCTGCATCTGTAGGAGACAGAGTCACCA | 2966 | DIQMTQSPSTLSASVGDRVTITCRASQS | 3249 | | |
| | TCACTTGCCGGGCCAGTCAGAGTATTAGTAGGTGGTTGGCCTGGTATCAGCAGAAACCAGG | | ISRWLAWYQQKPGKAPKLLIYKASSLES | | | |
| | GAAAGCCCCTAAGCTCCTGATCTATAAGGCGTCTAGTTTAGAAAGTGGGGTCCCATCAAGG | | GVPSRFSGSGSGTEFTLTISSLQPDDFA | | | |
| | TTCAGCGGCAGTGGATCTGGGACAGAATTCACTCTCACCATCAGCAGCCTGCAGCCTGATG | | TYYCQQYDSYPWTFGQGTKVEIK | | | |
| | ATTTTGCAACTTATTACTGCCAACAGTATGATAGTTATCCGTGGACCTTCGGCCAAGGGAC | | | | | |
| | CAAGGTGGAAATCAAA | | | | | |
| ADI-75817 | GACATCCAGATGACCCAGTCTCCAAACTCCCTGCCCGTCACCCCTGGAGAGCCGGCCTCCA | 2967 | DIQMTQSPNSLPVTPGEPASISCRSSQS | 3250 | | |
| | TCTCCTGCAGGTCTAGTCAGAGCCTCCTGCACACTAATGGATACAACTATTTGGATTGGTA | | LLHTNGYNYLDWYLQKPGQSPQLLIYLG | | | |
| | CCTGCAGAAGCCAGGGCAGTCTCCACAGCTCCTGATCTATTTGGGTTCTAATCGGGCCTCC | | SNRASGVPDRFSGSGSGTDFTLKISRVE | | | |
| | GGGGTCCCTGACAGGTTCAGTGGCAGTGGATCAGGCACAGATTTTACACTGAAAATCAGCA | | AEDVGVYYCMQGLQTPPTFGGGTKVEIK | | | |
| | GAGTGGAGGCTGAGGATGTTGGGGTTTATTACTGCATGCAAGGTCTACAAACTCCTCCCAC | | | | | |
| | TTTCGGCGGAGGGACCAAGGTGGAGATCAAA | | | | | |
| ADI-75590 | GAAATCCAAATGACCCAGTCTCCTTCCTCCCTGTCTGCTTCTGTAGGAGACAGAGTCACCA | 2968 | EIQMTQSPSSLSASVGDRVTITCRASQG | 3251 | | |
| | TCACTTGCCGGGCAAGTCAGGGCATTGGCAGTGCTTTAGCCTGGTATCGGCAGAGGCCAGG | | IGSALAWYRQRPGKPPKLLISDASSLEG | | | |
| | GAAACCTCCTAAGCTCCTAATCTCTGATGCCTCCAGTTTGGAAGGTGGGGTCCCATCAAGG | | GVPSRFSGSGSGTDFTLTISSLQPEDFA | | | |
| | TTCAGCGGCAGTGGATCTGGGACAGATTTCACGCTCACCATCAGCAGCCTGCAGCCTGAAG | | TYYCQQFNNRITFGGGTKVEIK | | | |
| | ATTTTGCAACTTATTACTGTCAGCAGTTTAACAACCGGATAACTTTCGGCGGAGGGACCAA | | | | | |
| | GGTGGAAATCAAA | | | | | |
| ADI-75823 | GACATCACGATGACCCAGTCTCCATCCTCCCTGTCTGCATCTGTAGGAGACAGAGTCACCA | 2969 | DITMTQSPSSLSASVGDRVTITCRASQS | 3252 | | |
| | TCACTTGCCGGGCAAGTCAGAGCATTAGAAGATTTAAATTGGTATCAGCAGAAACCAGG | | IRRFLNWYQQKPGKAPKLLIYDASSLQS | | | |
| | GAAAGCCCCTAAGCTCCTGATCTATGATGCATCCAGTTTGCAAAGTGGGGTCCCATCAAGG | | GVPSRFSGSGSGTDFTLTISSLQPEDFA | | | |
| | TTCAGTGGCAGTGGATCTGGGACAGATTTCACTCTCACCATCAGCAGTCTGCAACCTGAAG | | TYYCQQSYNTPPWTFGQGTKVEIK | | | |
| | ATTTTGCAACTTACTACTGTCAGCAGAGTTACAATACCCCCCCGTGGACGTTCGGCCAAGG | | | | | |
| | GACCAAGGTGGAAATCAAA | | | | | |
| ADI-75569 | TCGTATGTGCTGACTCAGCCACCCTCGGTGTCAGTGGCCCCAGGACAGACGGCCAGGATTA | 2970 | SYVLTQPPSVSVAPGQTARITCEGNYIG | 3253 | | |
| | CCTGTGAGGGAAACTACATTGGAGGTCAAAGTGTAAACTGGTACCAGCAGAGGCCAGGCCA | | GQSVNWYQQRPGQAPVVVLYDDFGRPSG | | | |
| | GGCCCCTGTGGTGGTCCTCTATGATGATTTCGGCCGGCCCTCAGGGATCCCTGAGCGATTC | | IPERFSGSNSGNTATLTINWVEAGDEAD | | | |
| | TCTGGCTCCAACTCTGGGAACACGGCCACCCTGACCATCAACTGGGTCGAAGCCGGGGATG | | YYCQVWDTSSDRILFGGGTKLTVL | | | |
| | AGGCCGACTATTATTGTCAGGTGTGGGATACTAGTAGTGACCGAATACTGTTCGGCGGAGG | | | | | |
| | GACCAAGCTGACCGTCCTA | | | | | |
| ADI-75623 | GACATGACAATGACGCAGTCTCCATCCTCCCTGTCTGCATCTGTAGGAGACAGAGTCACCA | 2971 | DMTMTQSPSSLSASVGDRVTITCRASQS | 3254 | | |
| | TCACTTGCCGGGCAAGTCAGAGCATTAGCAAGTTTTTAAATTGGTATCAGCAGAAACCAGG | | ISKFLNWYQQKPGKAPNLLIYAASSLQS | | | |
| | GAAAGCCCCTAATCTCCTGATCTATGCTGCATCCAGTTTGCAAAGTGGGGTCCCATCAAGG | | GVPSRFSGSGSGTDFTLTINSLQPEDFA | | | |
| | TTCAGTGGCAGTGGATCTGGGACAGATTTCACTCTCACCATCAACAGTCTGCAACCTGAAG | | TYYCQQSYTMPPVTFGQGTRLEIK | | | |
| | ATTTTGCAACTTACTACTGTCAACAGAGTTACACTATGCCTCCGGTCACCTTCGGCCAAGG | | | | | |
| | GACACGACTGGAGATTAAA | | | | | |
| ADI-75626 | GACATCCAGATGACCCAGTCTCCATCCTCCCTGTCTGCGTCTGTCGGAGACAGAGTCACCA | 2972 | DIQMTQSPSSLSASVGDRVTITCRASQS | 3255 | | |
| | TCACTTGCCGGGCAAGTCAGAGCATTAGCGACTTTTTAAGTTGGTTCCAGCAGAGACCAGG | | ISDFLSWFQQRPGKAPKLLIYAASTLQS | | | |
| | GAAAGCCCCTAAGCTCCTGATCTATGCTGCATCAACTTTACAAAGTGGGGTCCCATCGAGG | | GVPSRFSGSGSGTDFTLTISSLQPEDFA | | | |
| | TTCAGTGGCAGTGGATCTGGGACAGATTTCACTCTCACCATCAGCAGTCTGCAACCTGAAG | | TYYCQQSFVTPYTFGRGTKVEIK | | | |
| | ATTTTGCAACTTACTACTGTCAACAGAGTTTCGTAACCCCGTACACTTTTGGCCGGGGGAC | | | | | |
| | CAAGGTGGAAATCAAA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75622 | IGKV1-12 | DIQMTQ | 1885 | RASQGLS | 2059 | WYQQKP | 2257 | AASSLQ | 2391 | GVPSRFSG | 2534 | QQANS | 2710 | FGGGTK | 2936 |
| | | SPSSVS | | RWLA | | GKAPKL | | S | | SGSGTDFT | | FPLT | | VEIK | |
| | | ASVGDR | | | | LIY | | | | LTISSLQP | | | | | |
| | | VTITC | | | | | | | | EDFATYYC | | | | | |
| ADI-75598 | IGKV1-33 | DIKLTQ | 1886 | QASHDIN | 2060 | WYQQKP | 2257 | EASNLE | 2393 | GVPSRFSG | 2537 | QQYDN | 2711 | FGQGTR | 2940 |
| | | SPSSLS | | NYLN | | GKAPKL | | T | | SGSGTDFT | | LPIT | | LEIK | |
| | | ASVGDR | | | | LIY | | | | FTISSLQP | | | | | |
| | | VTITC | | | | | | | | EDIATYYC | | | | | |
| ADI-75572 | IGLV2-8 | ELVLTQ | 1887 | TGTSSDV | 2061 | WYQQHP | 2265 | EVNKRP | 2394 | GVPDRFSG | 2538 | SSYAG | 2712 | FGGGTK | 2939 |
| | | SPSASG | | GGYNYVS | | GKAPKL | | S | | SKSGNTAS | | PFWV | | LTVL | |
| | | SPGQSV | | | | MIY | | | | LTVSGLQA | | | | | |
| | | TISC | | | | | | | | EDEADYYC | | | | | |
| ADI-75819 | IGKV1- | DIQMTQ | 1878 | RASQGIS | 2062 | WYQQKP | 2259 | SASTLE | 2395 | GVPSRFSG | 2539 | QEYYS | 2713 | FGPGTK | 2942 |
| | NL1 | SPSSLS | | NSLA | | GKAPKL | | S | | SGSGTDFT | | TRT | | VEIK | |
| | | ASVGDR | | | | LLY | | | | LTITSLQP | | | | | |
| | | VTITC | | | | | | | | EDFATYYC | | | | | |
| ADI-75616 | IGKV1D- | DIQMTQ | 1878 | RASQGIN | 2063 | WYQQKP | 2259 | DASSLE | 2396 | GVPSRFSG | 2540 | QQFNN | 2714 | FGGGTK | 2938 |
| | 13 | SPSSLS | | SALA | | GKAPKL | | N | | GGSGTDFT | | LIT | | VDIK | |
| | | ASVGDR | | | | LLY | | | | LTITSLQP | | | | | |
| | | VTITC | | | | | | | | EDFATYYC | | | | | |
| ADI-75581 | IGKV1D- | EIQMTQ | 1881 | RAGQSIA | 2064 | WYQQKP | 2257 | AASSLQ | 2391 | GVPSRFSG | 2541 | QQSYS | 2715 | FGQGTR | 2940 |
| | 39 | SPSSLS | | TYLN | | GKAPKL | | S | | SGSGADFT | | TPIT | | LEIK | |
| | | ASVGDR | | | | LIY | | | | LTISSLQP | | | | | |
| | | VTITC | | | | | | | | EDFATYYC | | | | | |
| ADI-75613 | IGKV1-33 | DIQLTQ | 1876 | QASQDIN | 2065 | WYQQKP | 2266 | DASNLE | 2397 | GVPSRFSG | 2542 | QQYDN | 2716 | FGGGTK | 2936 |
| | | SPSSLS | | NYLI | | GKAPRV | | A | | SGSGTDFT | | VPLI | | VEIK | |
| | | ASVGDR | | | | LIY | | | | FTISSLQP | | | | | |
| | | VTITC | | | | | | | | EDIATYFC | | | | | |
| ADI-75872 | IGKV1D- | EIVLTQ | 1888 | RASQSIS | 2066 | WYQQKP | 2257 | AASSLQ | 2391 | GVPSRFSG | 2543 | QQSYT | 2717 | FGQGTK | 2937 |
| | 39 | SPSSLS | | TYLN | | GKAPKL | | S | | SGSGTDFT | | APGYT | | VEIK | |
| | | ASVGDR | | | | LIY | | | | LTVSSLQP | | | | | |
| | | VTITC | | | | | | | | EDFATYFC | | | | | |
| VYD223 | IGKV1- | EIVMTQ | 1889 | RASQGIT | 2067 | WYQQKP | 2267 | AASTLE | 2398 | GVPPRFRG | 2544 | QQYFD | 2718 | FGQGTK | 2937 |
| (ADI- | NL1 | SPSSLS | | NSLA | | GKAPNL | | S | | SGSGTDFT | | SPHT | | VEIK | |
| 75865) | | ASVGDT | | | | LLY | | | | LTISSLQP | | | | | |
| | | VTITC | | | | | | | | EDFATFYC | | | | | |
| ADI-75822 | IGLV3-21 | EIVLTQ | 1890 | GGNNIGS | 2068 | WLQQKP | 2268 | DDSDRP | 2399 | GIPERFSG | 2545 | QVWDS | 2719 | FGTGTQ | 2943 |
| | | SPSVSV | | RSVH | | GQAPVL | | S | | SNSGNTAT | | YSNND | | LTVL | |
| | | APGQTA | | | | VVY | | | | LTISRVEA | | V | | | |
| | | RITC | | | | | | | | GDEADYYC | | | | | |
| ADI-75843 | IGKV1- | EIVMTQ | 1891 | RASQGIS | 2062 | WYQQKP | 2259 | AASTLE | 2398 | GVPSRFSG | 2531 | QQYYS | 2720 | FGQGTK | 2944 |
| | NL1 | SPSSLS | | NSLA | | GKAPKL | | S | | SGSGTDYT | | TRS | | VDIK | |
| | | ASVGDR | | | | LLY | | | | LTISSLQP | | | | | |
| | | VTITC | | | | | | | | EDFATYYC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75622 | GACATCCAGATGACCCAGTCTCCATCTTCCGTGTCTGCATCTGTGGGAGACAGAGTCACAA | 2973 | DIQMTQSPSSVSASVGDRVTITCRASQG | 3256 | | |
| | TCACTTGTCGGGCGAGTCAGGGTCTTAGTAGATGGTTAGCCTGGTATCAGCAGAAACCAGG | | LSRWLAWYQQKPGKAPKLLIYAASSLQS | | | |
| | GAAAGCCCCTAAGCTCCTGATCTATGCTGCATCTAGTTTGCAAAGTGGGGTCCCGTCAAGG | | GVPSRFSGSGSGTDFTLTISSLQPEDFA | | | |
| | TTCAGCGGCAGTGGATCTGGGACAGATTTCACTCTCACCATCAGCAGCCTGCAGCCTGAAG | | TYYCQQANSFPLTFGGGTKVEIK | | | |
| | ACTTTGCAACTTACTATTGTCAACAGGCTAACAGTTTCCCCCTCACTTTCGGCGGTGGGAC | | | | | |
| | CAAGGTGGAAATCAAA | | | | | |
| ADI-75598 | GACATCAAATTGACCCAGTCTCCATCCTCCCTGTCTGCCTCTGTAGGAGACAGAGTCACCA | 2974 | DIKLTQSPSSLSASVGDRVTITCQASHD | 3257 | | |
| | TCACTTGCCAGGCGAGTCACGACATTAACAACTATTTAAATTGGTATCAGCAAAAACCAGG | | INNYLNWYQQKPGKAPKLLIYEASNLET | | | |
| | GAAAGCCCCTAAACTCCTGATCTACGAAGCATCCAACTTGGAGACAGGGGTCCCATCAAGA | | GVPSRFSGSGSGTDFTFTISSLQPEDIA | | | |
| | TTCAGTGGAAGTGGATCTGGGACAGACTTTACTTTCACCATCAGCAGCCTGCAGCCTGAAG | | TYYCQQYDNLPITFGQGTRLEIK | | | |
| | ATATTGCAACATATTACTGTCAACAATATGATAATCTCCCCATCACCTTCGGCCAAGGGAC | | | | | |
| | ACGACTGGAGATTAAA | | | | | |
| ADI-75572 | GAGCTTGTGCTGACTCAGTCCCCCTCCGCGTCCGGGTCTCCTGGACAGTCAGTCACCATCT | 2975 | ELVLTQSPSASGSPGQSVTISCTGTSSD | 3258 | | |
| | CCTGCACTGGAACCAGTAGTGACGTTGGTGGTTATAACTATGTCTCCTGGTACCAACAACA | | VGGYNYVSWYQQHPGKAPKLMIYEVNKR | | | |
| | CCCCGGCAAAGCCCCCAAACTCATGATTTATGAGGTCAATAAGCGGCCCTCAGGGGTCCCT | | PSGVPDRFSGSKSGNTASLTVSGLQAED | | | |
| | GATCGTTTCTCTGGCTCCAAGTCTGGCAACACGGCCTCCCTGACCGTCTCTGGGCTCCAGG | | EADYYCSSYAGPFWVFGGGTKLTVL | | | |
| | CTGAGGATGAGGCTGATTATTATTGTAGCTCATATGCAGGCCCCTTTTGGGTGTTCGGCGG | | | | | |
| | AGGCACCAAGCTCACCGTCCTA | | | | | |
| ADI-75819 | GACATCCAGATGACCCAGTCTCCATCCTCCCTGTCTGCATCTGTGGGAGACAGAGTCACCA | 2976 | DIQMTQSPSSLSASVGDRVTITCRASQG | 3259 | | |
| | TCACTTGCCGGGCGAGTCAGGGCATTAGTAATTCTTTAGCCTGGTATCAGCAGAAACCAGG | | ISNSLAWYQQKPGKAPKLLLYSASTLES | | | |
| | GAAAGCCCCTAAGCTCCTGCTCTATTCTGCATCCACATTGGAAAGTGGGGTCCCATCCAGG | | GVPSRFSGSGSGTDFTLTITSLQPEDFA | | | |
| | TTCAGTGGCAGTGGATCTGGGACGGATTTCACTCTCACCATCACCAGCCTGCAGCCTGAAG | | TYYCQEYYSTRTFGPGTKVEIK | | | |
| | ATTTTGCAACTTATTACTGTCAAGAGTATTATAGTACCCGGACTTTCGGCCCTGGGACCAA | | | | | |
| | GGTGGAGATCAAA | | | | | |
| ADI-75616 | GACATCCAGATGACCCAGTCTCCATCCTCCCTGTCTGCATCTGTAGGAGACAGAGTCACCA | 2977 | DIQMTQSPSSLSASVGDRVTITCRASQG | 3260 | | |
| | TCACTTGCCGGGCAAGTCAGGGCATTAACAGTGCTTTAGCCTGGTATCAGCAGAAACCAGG | | INSALAWYQQKPGKAPKLLLYDASSLEN | | | |
| | GAAAGCTCCTAAACTCCTGCTCTATGATGCCTCCAGTTTGGAAAATGGGGTCCCATCAAGG | | GVPSRFSGGGSGTDFTLTITSLQPEDFA | | | |
| | TTCAGCGGCGGTGGATCTGGGACAGATTTCACTCTCACCATCACCAGCCTGCAGCCTGAAG | | TYYCQQFNNLITFGGGTKVDIK | | | |
| | ATTTTGCAACTTATTATTGTCAACAATTTAATAATCTGATCACTTTCGGCGGAGGGACCAA | | | | | |
| | AGTGGATATCAAA | | | | | |
| ADI-75581 | GAAATCCAGATGACCCAGTCTCCATCCTCCCTGTCTGCATCTGTAGGAGACAGAGTCACCA | 2978 | EIQMTQSPSSLSASVGDRVTITCRAGQS | 3261 | | |
| | TCACTTGCCGGGCAGGTCAGAGCATTGCCACCTATTTAAATTGGTATCAGCAGAAACCAGG | | IATYLNWYQQKPGKAPKLLIYAASSLQS | | | |
| | GAAAGCCCCTAAGCTCCTGATCTATGCTGCATCCAGTTTGCAAAGTGGGGTCCCATCAAGG | | GVPSRFSGSGSGADFTLTISSLQPEDFA | | | |
| | TTCAGTGGCAGTGGATCTGGGGCAGATTTCACTCTCACCATCAGCAGTCTGCAACCTGAAG | | TYYCQQSYSTPITFGQGTRLEIK | | | |
| | ATTTTGCAACTTACTACTGTCAACAGAGTTACAGTACCCCTATCACCTTCGGCCAAGGGAC | | | | | |
| | ACGACTGGAGATTAAA | | | | | |
| ADI-75613 | GACATCCAATTGACCCAGTCTCCATCCTCCCTGTCTGCATCTGTAGGAGACAGAGTCACCA | 2979 | DIQLTQSPSSLSASVGDRVTITCQASQD | 3262 | | |
| | TCACTTGCCAGGCGAGTCAGGACATTAACAACTATTTAATTTGGTATCAGCAAAAACCAGG | | INNYLIWYQQKPGKAPRVLIYDASNLEA | | | |
| | CAAAGCCCCTAGGGTCCTGATCTACGATGCATCCAATTTGGAAGCAGGGGTCCCATCAAGG | | GVPSRFSGSGSGTDFTFTISSLQPEDIA | | | |
| | TTCAGTGGAAGTGGATCTGGGACAGATTTTACTTTCACCATCAGCAGCCTGCAGCCTGAAG | | TYFCQQYDNVPLIFGGGTKVEIK | | | |
| | ATATTGCTACATATTTCTGTCAACAATATGATAATGTCCCCCTCATTTTCGGCGGAGGGAC | | | | | |
| | CAAGGTGGAAATCAAA | | | | | |
| ADI-75872 | GAAATCGTGTTGACCCAGTCTCCATCCTCCCTGTCTGCATCTGTTGGAGACAGAGTCACCA | 2980 | EIVLTQSPSSLSASVGDRVTITCRASQS | 3263 | | |
| | TCACTTGCCGGGCAAGTCAGAGCATTAGCACCTATTTAAATTGGTATCAGCAGAAACCAGG | | ISTYLNWYQQKPGKAPKLLIYAASSLQS | | | |
| | GAAAGCCCCTAAGCTCCTGATCTATGCTGCATCCAGTTTGCAAAGTGGGGTCCCATCAAGG | | GVPSRFSGSGSGTDFTLTVSSLQPEDFA | | | |
| | TTCAGTGGCAGTGGGTCTGGGACAGATTTCACTCTCACCGTCAGCAGTCTGCAACCTGAAG | | TYFCQQSYTAPGYTFGQGTKVEIK | | | |
| | ATTTTGCAACTTACTTCTGTCAACAGAGTTACACTGCCCCTGGGTACACTTTTGGCCAGGG | | | | | |
| | GACCAAGGTGGAGATCAAA | | | | | |
| VYD223 | GAAATTGTGATGACCCAGTCTCCATCCTCCCTGTCTGCATCTGTAGGAGACACAGTCACCA | 2981 | EIVMTQSPSSLSASVGDTVTITCRASQG | 3264 | DIQMTQSPS | 3525 |
| (ADI- | TCACTTGCCGGGCGAGTCAGGGCATTACCAATTCCTTAGCCTGGTATCAGCAGAAACCAGG | | ITNSLAWYQQKPGKAPNLLLYAASTLES | | SLSASVGDT | |
| 75865) | GAAAGCCCCTAACCTCCTGCTCTATGCCGCATCCACATTGGAAAGTGGGGTCCCACCCAGG | | GVPPRFRGSGSGTDFTLTISSLQPEDFA | | VTITCRASQ | |
| | TTCCGTGGCAGCGGATCTGGGACGGATTTCACTCTCACCATCAGCAGCCTGCAGCCTGAAG | | TFYCQQYFDSPHTFGQGTKVEIK | | GITNSLAWY | |
| | ATTTTGCAACTTTTTACTGTCAACAGTATTTTGATAGCCCTCACACTTTTGGCCAGGGGAC | | | | QQKPGKAPN | |
| | CAAGGTGGAAATCAAA | | | | LLLYAASTL | |
| | | | | | ESGVPPRFR | |
| | | | | | GSGSGTDFT | |
| | | | | | LTISSLQPE | |
| | | | | | DFATFYCQQ | |
| | | | | | YFDSPHTFG | |
| | | | | | QGTKVEIK | |
| ADI-75822 | GAAATTGTGTTGACGCAGTCTCCCTCGGTGTCAGTGGCCCCAGGACAGACGGCCAGGATTA | 2982 | EIVLTQSPSVSVAPGQTARITCGGNNIG | 3265 | | |
| | CCTGTGGGGGAAACAACATTGGAAGTAGAAGTGTGCACTGGCTCCAGCAGAAGCCAGGCCA | | SRSVHWLQQKPGQAPVLVVYDDSDRPSG | | | |
| | GGCCCCTGTGCTGGTCGTCTATGATGATAGCGACCGGCCCTCAGGGATCCCTGAGCGATTC | | IPERFSGSNSGNTATLTISRVEAGDEAD | | | |
| | TCAGGCTCCAACTCTGGGAACACGGCCACCCTGACCATCAGCAGGGTCGAAGCCGGGGATG | | YYCQVWDSYSNNDVFGTGTQLTVL | | | |
| | AGGCCGACTATTACTGTCAGGTGTGGGATAGTTATAGTAATAACGATGTCTTCGGAACTGG | | | | | |
| | GACCCAGCTGACCGTCCTC | | | | | |
| ADI-75843 | GAAATTGTGATGACCCAGTCTCCATCCTCCCTGTCTGCATCTGTAGGAGACAGAGTCACCA | 2983 | EIVMTQSPSSLSASVGDRVTITCRASQG | 3266 | | |
| | TCACTTGCCGGGCGAGTCAGGGCATTAGCAATTCTTTGGCCTGGTATCAGCAGAAACCAGG | | ISNSLAWYQQKPGKAPKLLLYAASTLES | | | |
| | GAAAGCCCCTAAGCTCCTGCTCTATGCTGCATCCACGTTGGAAAGTGGGGTCCCATCCAGG | | GVPSRFSGSGSGTDYTLTISSLQPEDFA | | | |
| | TTCAGTGGCAGTGGATCTGGGACGGATTACACTCTCACCATCAGCAGCCTGCAGCCTGAAG | | TYYCQQYYSTRSFGQGTKVDIK | | | |
| | ATTTTGCAACTTATTACTGTCAACAGTATTATAGTACCAGGTCGTTCGGCCAAGGGACCAA | | | | | |
| | GGTGGATATCAAA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75584 | IGLV3-25 | TYELTQ | 1892 | SGDSLPR | 2069 | WYQQKP | 2269 | KDSERP | 2400 | GIPERFSG | 2546 | QSADS | 2721 | FGGGTK | 2939 |
| | | PPSVSV | | QYAY | | GQAPVM | | S | | STSGTTVT | | GSYHV | | LTVL | |
| | | SPGQTA | | | | VIH | | | | LTISGVQA | | I | | | |
| | | RITC | | | | | | | | EDEADYYC | | | | | |
| ADI-75655 | IGKV1D- | DIQMTQ | 1878 | RAGQGIG | 2070 | WYQQKP | 2259 | DASSLE | 2396 | GVPSRFSG | 2534 | QQFNK | 2722 | FGGGTK | 2938 |
| | 13 | SPSSLS | | TALA | | GKAPKL | | N | | SGSGTDFT | | LVT | | VDIK | |
| | | ASVGDR | | | | LLY | | | | LTISSLQP | | | | | |
| | | VTITC | | | | | | | | EDFATYYC | | | | | |
| ADI-75641 | IGLV3-21 | SHVLTQ | 1893 | GEDDIGS | 2071 | WYQQKP | 2270 | YDRDRP | 2401 | GIPERFSG | 2547 | QVWDN | 2723 | FGGGTK | 2939 |
| | | PPSVSV | | KSVH | | GQAPML | | S | | SNSGNTAT | | SWV | | LTVL | |
| | | APGKTA | | | | IIY | | | | LTISSVEA | | | | | |
| | | KITC | | | | | | | | GDEADYYC | | | | | |
| ADI-75565 | IGKV1-13 | DIVMTQ | 1894 | RASQDIG | 2072 | WYQQKP | 2271 | DASSLG | 2402 | GVPSRFSG | 2548 | QQFNS | 2724 | FGGGTK | 2936 |
| | | SPSSLS | | SALA | | GNAPRF | | S | | SGSGTDFT | | LVT | | VEIK | |
| | | ASVGDS | | | | LIS | | | | LTIRGLQP | | | | | |
| | | TITC | | | | | | | | ADFATYYC | | | | | |
| ADI-75783 | IGKV1D- | DIQMTQ | 1895 | RASQTIN | 2073 | WYQQKP | 2257 | GASSLQ | 2403 | GVPSRFSG | 2549 | QQTYS | 2725 | FGPGTK | 2942 |
| | 39 | SPSSLS | | TYLN | | GKAPKL | | S | | SGSGTDFT | | TPGVT | | VEIK | |
| | | ASVGDR | | | | LIY | | | | LSISGVQP | | | | | |
| | | VTISC | | | | | | | | EDFATYYC | | | | | |
| ADI-75805 | IGKV1D- | DIQMTQ | 1896 | RASQSMT | 2074 | WYQQKP | 2272 | GASSLH | 2404 | GVPSRFSG | 2534 | QQSYS | 2726 | FGQGTK | 2937 |
| | 39 | SPSSLS | | NSLN | | GKAPKL | | S | | SGSGTDFT | | NPWT | | VEIK | |
| | | ASVGDR | | | | LIH | | | | LTISSLQP | | | | | |
| | | VTIAC | | | | | | | | EDFATYYC | | | | | |
| ADI-75588 | IGKV1- | DIQMTQ | 1878 | RASQGIS | 2062 | WYQQQP | 2273 | AVSTLE | 2405 | GVPSRFSG | 2550 | QEYYS | 2713 | FGQGTK | 2937 |
| | NL1 | SPSSLS | | NSLA | | GKAPKL | | S | | SGSGTDYT | | TRT | | VEIK | |
| | | ASVGDR | | | | LLY | | | | LTINSLQP | | | | | |
| | | VTITC | | | | | | | | EDFATYYC | | | | | |
| ADI-75871 | IGKV1- | DIQMTQ | 1897 | RASQDIT | 2075 | WYQQRP | 2274 | AASTLE | 2398 | GVPSRFTG | 2551 | QQYYS | 2727 | FGQGTK | 2937 |
| | NL1 | SPSSLS | | NSLA | | GKAPKL | | S | | SGSGTDFT | | ALRRT | | VEIK | |
| | | ASVGDR | | | | LLY | | | | LTISSLQP | | | | | |
| | | TITC | | | | | | | | EDFATYYC | | | | | |
| ADI-75868 | IGKV1- | EIPLTQ | 1898 | RASQGIR | 2076 | WYQQKP | 2259 | AASRLQ | 2406 | GVPSRFSG | 2531 | QQYYN | 2728 | FGQGTK | 2937 |
| | NL1 | SPSSLS | | NSLA | | GKAPKL | | S | | SGSGTDYT | | TPPWT | | VEIK | |
| | | ASVGDR | | | | LLY | | | | LTISSLQP | | | | | |
| | | VTITC | | | | | | | | EDFATYYC | | | | | |
| ADI-75653 | IGKV1D- | DIQMTQ | 1899 | RASQSIS | 2050 | WYQQKP | 2257 | GASSLH | 2407 | GVPSRFSG | 2534 | QQSYS | 2729 | FGQGTK | 2937 |
| | 39 | SPSSLP | | SYLN | | GKAPKL | | T | | SGSGTDFT | | TPRT | | VEIK | |
| | | ASVGDR | | | | LIY | | | | LTISSLQP | | | | | |
| | | VTITC | | | | | | | | EDFATYYC | | | | | |
| ADI-75826 | IGKV1D- | EIVLTQ | 1888 | RASQSIS | 2077 | WYQQKP | 2257 | AASSLQ | 2391 | GVPSRFSG | 2552 | QQSYN | 2702 | FGQGTK | 2937 |
| | 39 | SPSSLS | | NYLN | | GKAPKL | | S | | SGSGTDFT | | TPPWT | | VEIK | |
| | | ASVGDR | | | | LIY | | | | LTISSLQP | | | | | |
| | | VTITC | | | | | | | | EDLATYYC | | | | | |
| ADI-75709 | IGKV1D- | DITLTQ | 1900 | RASQSIS | 2078 | WYQQKP | 2275 | AASNLQ | 2408 | GVPSRFSG | 2534 | QQSYN | 2702 | FGPGTK | 2942 |
| | 39 | SPSSLS | | RHLS | | GRAPKL | | S | | SGSGTDFT | | TPPWT | | VEIK | |
| | | ASVGDR | | | | LIY | | | | LTISSLQP | | | | | |
| | | VTITC | | | | | | | | EDFATYYC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75584 | ACCTATGAGCTGACTCAGCCACCCTCGGTGTCAGTGTCCCCAGGACAGACGGCCAGGATCA | 2984 | TYELTQPPSVSVSPGQTARITCSGDSLP | 3267 | | |
| | CCTGCTCTGGAGATTCATTGCCAAGGCAATATGCTTATTGGTACCAACAGAAGCCAGGCCA | | RQYAYWYQQKPGQAPVMVIHKDSERPSG | | | |
| | GGCCCCTGTGATGGTAATACATAAAGACAGTGAGAGGCCCTCAGGGATCCCTGAGCGATTC | | IPERFSGSTSGTTVTLTISGVQAEDEAD | | | |
| | TCTGGCTCCACCTCAGGGACAACAGTCACGTTGACCATCAGTGGAGTCCAGGCAGAAGACG | | YYCQSADSGSYHVIFGGGTKLTVL | | | |
| | AGGCTGACTATTACTGTCAATCAGCAGACAGTGGTAGTTATCATGTGATATTCGGCGGAGG | | | | | |
| | GACCAAGCTGACCGTCCTA | | | | | |
| ADI-75655 | GACATCCAGATGACCCAGTCTCCATCCTCCCTGTCTGCATCTGTAGGAGACAGAGTCACCA | 2985 | DIQMTQSPSSLSASVGDRVTITCRAGQG | 3268 | | |
| | TCACTTGCCGGGCAGGTCAGGGCATTGGCACAGCTTTAGCCTGGTATCAGCAGAAACCAGG | | IGTALAWYQQKPGKAPKLLLYDASSLEN | | | |
| | GAAAGCTCCTAAGCTCCTGCTCTATGATGCCTCCAGTTTGGAAAATGGGGTCCCATCCAGG | | GVPSRFSGSGSGTDFTLTISSLQPEDFA | | | |
| | TTCAGCGGCAGTGGATCTGGGACAGATTTCACTCTCACCATCAGCAGCCTGCAGCCTGAAG | | TYYCQQFNKLVTFGGGTKVDIK | | | |
| | ATTTTGCAACTTACTACTGTCAACAGTTTAATAAATTGGTCACTTTTGGCGGAGGGACCAA | | | | | |
| | GGTGGATATCAAA | | | | | |
| ADI-75641 | TCGCATGTGCTGACTCAGCCACCCTCCGTGTCAGTGGCCCCAGGAAAGACGGCCAAGATTA | 2986 | SHVLTQPPSVSVAPGKTAKITCGEDDIG | 3269 | | |
| | CCTGTGGGGAAGACGACATTGGAAGTAAAAGTGTGCACTGGTACCAGCAGAAGCCAGGCCA | | SKSVHWYQQKPGQAPMLIIYYDRDRPSG | | | |
| | GGCCCCTATGCTGATCATCTATTATGATAGGGACCGGCCCTCAGGGATCCCTGAGCGATTC | | IPERFSGSNSGNTATLTISSVEAGDEAD | | | |
| | TCTGGCTCCAACTCTGGGAACACGGCCACCCTGACCATCAGCAGCGTCGAAGCCGGGGATG | | YYCQVWDNSWVFGGGTKLTVL | | | |
| | AGGCCGACTATTACTGTCAGGTGTGGGATAATAGTTGGGTGTTCGGCGGAGGGACCAAGCT | | | | | |
| | CACCGTCCTA | | | | | |
| ADI-75565 | GACATCGTGATGACCCAGTCTCCGTCCTCCCTGTCTGCTTCTGTGGGGGACAGCATCACCA | 2987 | DIVMTQSPSSLSASVGDSITITCRASQD | 3270 | | |
| | TCACTTGCCGGGCAAGTCAGGACATTGGCAGTGCTTTAGCCTGGTATCAGCAGAAACCGGG | | IGSALAWYQQKPGNAPRFLISDASSLGS | | | |
| | GAATGCTCCTAGGTTCCTGATCTCTGATGCCTCCAGTTTGGGAAGTGGGGTCCCATCACGG | | GVPSRFSGSGSGTDFTLTIRGLQPADFA | | | |
| | TTCAGCGGCAGTGGATCTGGGACAGATTTCACTCTCACCATCAGAGGCCTGCAGCCTGCAG | | TYYCQQFNSLVTFGGGTKVEIK | | | |
| | ATTTTGCAACTTATTACTGTCAACAGTTTAATAGCCTGGTCACTTTCGGCGGAGGGACCAA | | | | | |
| | GGTGGAAATCAAA | | | | | |
| ADI-75783 | GACATCCAGATGACCCAGTCTCCATCCTCCCTGTCTGCATCTGTAGGCGACAGAGTCACCA | 2988 | DIQMTQSPSSLSASVGDRVTISCRASQT | 3271 | | |
| | TCTCTTGCCGGGCAAGTCAGACCATTAACACCTATTTAAATTGGTATCAGCAGAAACCAGG | | INTYLNWYQQKPGKAPKLLIYGASSLQS | | | |
| | GAAAGCCCCTAAGCTCCTGATCTATGGTGCATCGAGTTTGCAAAGTGGGGTCCCGTCAAGA | | GVPSRFSGSGSGTDFTLSISGVQPEDFA | | | |
| | TTCAGTGGCAGTGGATCGGGGACAGATTTCACTCTCAGCATCAGCGGTGTCCAACCTGAAG | | TYYCQQTYSTPGVTFGPGTKVEIK | | | |
| | ACTTTGCAACTTACTATTGTCAACAGACTTACAGTACCCCTGGGGTCACTTTCGGCCCTGG | | | | | |
| | GACCAAGGTGGAAATCAAA | | | | | |
| ADI-75805 | GACATCCAGATGACCCAGTCTCCATCGTCCCTGTCTGCATCTGTAGGAGACAGAGTCACCA | 2989 | DIQMTQSPSSLSASVGDRVTIACRASQS | 3272 | | |
| | TCGCTTGCCGGGCAAGTCAGAGCATGACAAACTCTTTAAATTGGTATCAGCAGAAACCAGG | | MTNSLNWYQQKPGKAPKLLIHGASSLHS | | | |
| | AAAAGCCCCTAAGCTCCTGATCCATGGTGCATCCAGTTTGCATAGTGGGGTCCCATCAAGG | | GVPSRFSGSGSGTDFTLTISSLQPEDFA | | | |
| | TTCAGTGGCAGTGGATCTGGGACAGATTTCACTCTCACCATCAGCAGTCTGCAACCTGAAG | | TYYCQQSYSNPWTFGQGTKVEIK | | | |
| | ATTTTGCAACTTACTACTGTCAACAGAGTTACAGTAATCCGTGGACATTCGGCCAAGGGAC | | | | | |
| | CAAGGTGGAGATCAAA | | | | | |
| ADI-75588 | GACATCCAGATGACCCAGTCTCCATCTTCCCTGTCTGCATCTGTAGGAGACAGAGTCACCA | 2990 | DIQMTQSPSSLSASVGDRVTITCRASQG | 3273 | | |
| | TCACTTGCCGGGCGAGTCAGGGCATTAGCAATTCTTTAGCCTGGTATCAGCAGCAACCAGG | | ISNSLAWYQQQPGKAPKLLLYAVSTLES | | | |
| | GAAAGCCCCTAAGCTCCTGCTTTATGCTGTATCCACATTGGAAAGTGGGGTCCCATCCAGG | | GVPSRFSGSGSGTDYTLTINSLQPEDFA | | | |
| | TTCAGTGGCAGTGGATCTGGGACGGATTACACTCTCACCATCAACAGCCTGCAGCCTGAAG | | TYYCQEYYSTRTFGQGTKVEIK | | | |
| | ATTTTGCAACTTATTACTGTCAAGAGTATTATAGTACCCGGACGTTCGGTCAAGGGACCAA | | | | | |
| | GGTGGAGATCAAA | | | | | |
| ADI-75871 | GACATCCAGATGACCCAGTCTCCATCCTCCCTGTCTGCATCTGTCGGAGACAGAATCACCA | 2991 | DIQMTQSPSSLSASVGDRITITCRASQD | 3274 | | |
| | TCACTTGCCGGGCGAGTCAGGGCATTAGCAATTCTTTAGCCTGGTATCAGCAGCAACCAGG | | ITNSLAWYQQRPGKAPKLLLYAASTLES | | | |
| | GAAAGCCCCTAAGCTCCTGCTCTATGCTGCATCCACATTGGAAAGTGGGGTCCCATCCCGG | | GVPSRFTGSGSGTDFTLTISSLQPEDFA | | | |
| | TTCACTGGCAGTGGATCTGGGACGGATTTCACTCTCACCATCAGCAGCCTGCAGCCTGAAG | | TYYCQQYYSALRRTFGQGTKVEIK | | | |
| | ATTTTGCGACTTATTACTGTCAACAGTATTATAGTGCCCTCCGGAGGACTTTTGGCCAGGG | | | | | |
| | GACCAAGGTGGAGATCAAA | | | | | |
| ADI-75868 | GAAATCCCATTGACCCAGTCTCCATCCTCCCTGTCTGCTTCTGTAGGAGACAGAGTCACCA | 2992 | EIPLTQSPSSLSASVGDRVTITCRASQG | 3275 | | |
| | TCACTTGCCGGGCGAGTCAGGGCATTAGGAATTCTTTAGCCTGGTATCAGCAAAAACCAGG | | IRNSLAWYQQKPGKAPKLLLYAASRLQS | | | |
| | GAAAGCCCCTAAGCTCCTGCTCTATGCTGCATCCAGATTGCAAAGTGGGGTCCCATCCAGG | | GVPSRFSGSGSGTDYTLTISSLQPEDFA | | | |
| | TTCAGTGGCAGTGGATCTGGGACAGATTACACTCTCACCATCAGCAGCCTGCAGCCTGAAG | | TYYCQQYYNTPPWTFGQGTKVEIK | | | |
| | ATTTTGCAACTTATTACTGTCAACAGTATTATAATACCCCTCCGTGGACGTTCGGCCAAGG | | | | | |
| | GACCAAGGTGGAAATCAAA | | | | | |
| ADI-75653 | GACATCCAGATGACCCAGTCTCCATCCTCCCTGCCTGCATCTGTAGGAGACAGAGTCACCA | 2993 | DIQMTQSPSSLPASVGDRVTITCRASQS | 3276 | | |
| | TCACTTGCCGGGCAAGTCAGAGCATTAGCAGTTATTTAAATTGGTATCAGCAAAAACCAGG | | ISSYLNWYQQKPGKAPKLLIYGASSLHT | | | |
| | GAAAGCCCCTAAGCTCCTGATTTATGGTGCATCCAGTTTGCATACTGGGGTCCCATCAAGG | | GVPSRFSGSGSGTDFTLTISSLQPEDFA | | | |
| | TTCAGTGGCAGTGGATCTGGGACAGATTTCACTCTCACCATCAGCAGTCTGCAACCTGAAG | | TYYCQQSYSTPRTFGQGTKVEIK | | | |
| | ATTTTGCAACTTACTACTGTCAACAGAGTTACAGTACCCCTCGGACTTTTGGCCAGGGGAC | | | | | |
| | CAAGGTGGAAATCAAA | | | | | |
| ADI-75826 | GAAATTGTGCTGACGCAGTCTCCATCCTCCCTGTCTGCATCTGTAGGAGACAGAGTCACCA | 2994 | EIVLTQSPSSLSASVGDRVTITCRASQS | 3277 | | |
| | TCACTTGCCGGGCAAGTCAGAGCATTAGCAACTATTTAAATTGGTATCAGCAGAAACCAGG | | ISNYLNWYQQKPGKAPKLLIYAASSLQS | | | |
| | GAAAGCCCCTAAGCTCCTGATCTATGCTGCATCCAGTTTGCAAAGTGGGGTCCCATCAAGG | | GVPSRFSGSGSGTDFTLTISSLQPEDLA | | | |
| | TTCAGTGGCAGTGGATCTGGGACAGATTTCACTCTCACCATCAGCAGTCTGCAACCTGAAG | | TYYCQQSYNTPPWTFGQGTKVEIK | | | |
| | ATCTTGCAACTTACTACTGTCAACAGAGTTACAATACTCCTCCTTGGACGTTCGGCCAAGG | | | | | |
| | GACCAAGGTGGAAATCAAA | | | | | |
| ADI-75709 | GACATCACACTGACCCAGTCTCCATCCTCCCTGTCTGCATCTGTGGGAGACAGAGTCACCA | 2995 | DITLTQSPSSLSASVGDRVTITCRASQS | 3278 | | |
| | TCACTTGCCGGGCAAGTCAGAGCATTAGCAGACATTTGAGTTGGTATCAGCAGAAGCCAGG | | ISRHLSWYQQKPGRAPKLLIYAASNLQS | | | |
| | GAGAGCCCCTAAGCTCCTGATCTATGCTGCATCCAATTTGCAAAGTGGGGTCCCATCAAGG | | GVPSRFSGSGSGTDFTLTISSLQPEDFA | | | |
| | TTTAGTGGCAGTGGATCTGGGACAGATTTCACTCTCACCATCAGCAGTCTGCAACCTGAAG | | TYYCQQSYNTPPWTFGPGTKVEIK | | | |
| | ATTTTGCAACTTACTACTGTCAACAGAGTTACAATACCCCCCCGTGGACGTTCGGCCCAGG | | | | | |
| | GACCAAGGTGGAAATCAAA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75685 | IGLV2-23 | QPVLIQ | 1901 | TGTSSDV | 2061 | WYQQHP | 2276 | DVSKRP | 2409 | GASDRFSG | 2553 | CSYAG | 2730 | FGGGTK | 2939 |
| | | PASVSG | | GGYNYVS | | GKAPKV | | S | | SKSGNTAS | | SRGWL | | LTVL | |
| | | SPGQSI | | | | LTY | | | | LTISGLQA | | | | | |
| | | TISC | | | | | | | | EDEADYYC | | | | | |
| ADI-75831 | IGKV1D- | DIVMTQ | 1902 | RAGQSIS | 2079 | WYQQKP | 2257 | AVSTLQ | 2410 | GVPSRFSA | 2554 | QQSYS | 2731 | FGGGTK | 2936 |
| | 39 | SPSSLS | | DFLN | | GKAPKL | | S | | SGSGTDFA | | SPLT | | VEIK | |
| | | ASVGDR | | | | LIY | | | | LTISSLQP | | | | | |
| | | VTITC | | | | | | | | EDFATYYC | | | | | |
| ADI-75873 | IGKV1- | DIQMTQ | 1878 | RASQDIK | 2080 | WYQQKP | 2259 | VASRLE | 2411 | GVPSRFSG | 2555 | QQYYN | 2732 | FGQGTK | 2937 |
| | NL1 | SPSSLS | | YSLA | | GKAPKL | | S | | SGSGTDYS | | TPVT | | VEIK | |
| | | ASVGDR | | | | LLY | | | | LTISSLQP | | | | | |
| | | VTITC | | | | | | | | EDFATYYC | | | | | |
| ADI-75621 | IGLV3-21 | SYELTQ | 1903 | GGNNVGS | 2081 | WYQQRP | 2277 | DNDARP | 2412 | GIPERFSA | 2556 | QVWDS | 2733 | FGTGTK | 2945 |
| | | PPSVSV | | KTVH | | GQAPVL | | S | | YDSGNTAT | | GSDHY | | VTVL | |
| | | APGQTA | | | | VVY | | | | LTITRVEA | | V | | | |
| | | KITC | | | | | | | | GDEADYYC | | | | | |
| ADI-75755 | IGKV1D- | DIQMTQ | 1878 | RASQSIR | 2082 | WYQQKP | 2257 | DASSLQ | 2413 | GVPSRFSG | 2531 | QQSYN | 2702 | FGQGTK | 2937 |
| | 39 | SPSSLS | | NYLN | | GKAPKL | | R | | SGSGTDYT | | TPPWT | | VEIK | |
| | | ASVGDR | | | | LIY | | | | LTISSLQP | | | | | |
| | | VTITC | | | | | | | | EDFATYYC | | | | | |
| ADI-75702 | IGKV1-12 | DIQMTQ | 1885 | RASQGIS | 2083 | WYQQKP | 2257 | AASSLQ | 2391 | GVPSRFSG | 2534 | QQADS | 2734 | FGGGTK | 2936 |
| | | SPSSVS | | RWLA | | GKAPKL | | S | | SGSGTDFT | | FPLT | | VEIK | |
| | | ASVGDR | | | | LIY | | | | LTISSLQP | | | | | |
| | | VTITC | | | | | | | | EDFATYYC | | | | | |
| ADI-75778 | IGKV1D- | DIQMTQ | 1878 | RASQSIR | 2082 | WYQQKP | 2257 | DASSLQ | 2413 | GVPSRFSG | 2531 | QQSYN | 2702 | FGQGTK | 2937 |
| | 39 | SPSSLS | | NYLN | | GKAPKL | | R | | SGSGTDYT | | TPPWT | | VEIK | |
| | | ASVGDR | | | | LIY | | | | LTISSLQP | | | | | |
| | | VTITC | | | | | | | | EDFATYYC | | | | | |
| ADI-75575 | IGKV1-33 | DIQLTQ | 1876 | QASQDIT | 2084 | WYQQRP | 2278 | DASYLE | 2414 | GVPSRFSG | 2557 | QQYES | 2735 | FGGGTK | 2938 |
| | | SPSSLS | | KNLN | | GKAPKL | | T | | SGSGTDFT | | LPPT | | VDIK | |
| | | ASVGDR | | | | LIF | | | | ITISSLQP | | | | | |
| | | VTITC | | | | | | | | EDFATYFC | | | | | |
| ADI-75690 | IGLV5-52 | QPVLTQ | 1904 | MILSSGFS | 2085 | WYQQKP | 2279 | YHSDSN | 2415 | GVPSRFSG | 2558 | GTWHS | 2736 | FGGGTK | 2939 |
| | | PPSHSA | | VGDFWIR | | GNPPRY | | KGQGS | | SNDASANA | | NSKTQ | | LTVL | |
| | | SSGTSV | | | | LLY | | | | GILRISGL | | V | | | |
| | | RLTC | | | | | | | | QPEDEADY | | | | | |
| | | | | | | | | | | YC | | | | | |
| ADI-75735 | IGKV1-33 | DIQMTQ | 1878 | QASQHIS | 2086 | WYQHRP | 2280 | DASNLE | 2416 | GVPSRFSG | 2537 | QQYGS | 2737 | FGQGTR | 2940 |
| | | SPSSLS | | NYLN | | GKAPKL | | T | | SGSGTDFT | | LPIT | | LEIK | |
| | | ASVGDR | | | | LIY | | | | FTISSLQP | | | | | |
| | | VTITC | | | | | | | | EDIATYYC | | | | | |
| ADI-75859 | IGKV1- | ETTLTQ | 1905 | RASQGIS | 2062 | WYQQKP | 2259 | AASTLE | 2398 | GVPSRFSG | 2559 | QQYYS | 2738 | FGQGTI | 2946 |
| | NL1 | SPSSLS | | NSLA | | GKAPKL | | S | | SGSGTDFT | | SVRFT | | LDLN | |
| | | ASVGDR | | | | LLY | | | | LTIASLQP | | | | | |
| | | VTITC | | | | | | | | EDFATYYC | | | | | |
| ADI-75857 | IGKV1- | DIPMTQ | 1906 | RASQGIS | 2062 | WYQQKP | 2281 | AASTLE | 2398 | GVPSRFSG | 2534 | QQYYS | 2739 | FGQGTK | 2937 |
| | NL1 | SPSTLS | | NSLA | | GRAPKL | | S | | SGSGTDFT | | TPPRT | | VEIK | |
| | | ASVGDR | | | | LLY | | | | LTISSLQP | | | | | |
| | | VTITC | | | | | | | | EDFATYYC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75685 | CAGCCTGTGCTGATTCAGCCTGCCTCCGTGTCTGGGTCTCCTGGACAGTCGATCACCATCT | 2996 | QPVLIQPASVSGSPGQSITISCTGTSSD | 3279 | | |
| | CCTGCACTGGAACCAGCAGTGATGTTGGTGGTTACAATTATGTCTCCTGGTACCAACAACA | | VGGYNYVSWYQQHPGKAPKVLTYDVSKR | | | |
| | CCCAGGCAAAGCCCCCAAAGTCCTGACTTATGATGTCAGTAAGCGGCCCTCAGGGGCTTCT | | PSGASDRFSGSKSGNTASLTISGLQAED | | | |
| | GATCGCTTCTCTGGCTCCAAGTCTGGCAACACGGCCTCCCTGACAATCTCTGGGCTCCAGG | | EADYYCCSYAGSRGWLFGGGTKLTVL | | | |
| | CTGAGGACGAGGCTGACTATTACTGCTGCTCATATGCAGGTAGTAGAGGTTGGCTATTCGG | | | | | |
| | CGGAGGGACCAAGCTCACCGTCCTA | | | | | |
| ADI-75831 | GACATCGTGATGACCCAGTCTCCATCTTCCCTGTCTGCATCTGTAGGAGACAGAGTCACCA | 2997 | DIVMTQSPSSLSASVGDRVTITCRAGQS | 3280 | | |
| | TCACTTGCCGGGCAGGTCAGAGCATTAGCGACTTTTTAAATTGGTATCAGCAGAAACCAGG | | ISDFLNWYQQKPGKAPKLLIYAVSTLQS | | | |
| | GAAAGCCCCTAAGCTCCTGATCTATGCTGTATCCACTTTGCAAAGTGGGGTCCCTTCAAGG | | GVPSRFSASGSGTDFALTISSLQPEDFA | | | |
| | TTCAGTGCCAGTGGATCTGGGACAGATTTCGCTCTCACCATCAGCAGTCTCCAACCTGAAG | | TYYCQQSYSSPLTFGGGTKVEIK | | | |
| | ATTTTGCAACTTACTACTGTCAACAGAGTTACAGTTCCCCCCTCACTTTCGGCGGAGGGAC | | | | | |
| | CAAGGTGGAAATCAAA | | | | | |
| ADI-75873 | GACATCCAGATGACCCAGTCTCCATCCTCCCTGTCTGCATCTGTAGGAGACAGAGTCACCA | 2998 | DIQMTQSPSSLSASVGDRVTITCRASQD | 3281 | | |
| | TCACTTGCCGGGCGAGTCAGGACATTAAGTATTCTTTAGCCTGGTATCAGCAGAAACCAGG | | IKYSLAWYQQKPGKAPKLLLYVASRLES | | | |
| | GAAAGCCCCTAAGCTCCTCCTCTATGTTGCATCCAGATTGGAGAGTGGCGTCCCATCCAGG | | GVPSRFSGSGSGTDYSLTISSLQPEDFA | | | |
| | TTCAGTGGCAGTGGATCTGGGACGGATTACAGTCTCACCATCAGCAGCCTGCAGCCTGAAG | | TYYCQQYYNTPVTFGQGTKVEIK | | | |
| | ATTTTGCAACTTATTACTGTCAACAATATTATAATACCCCCGTGACGTTCGGCCAAGGGAC | | | | | |
| | CAAGGTGGAAATCAAA | | | | | |
| ADI-75621 | TCGTATGAGCTGACTCAGCCACCCTCGGTGTCAGTGGCCCCAGGACAGACGGCCAAGATTA | 2999 | SYELTQPPSVSVAPGQTAKITCGGNNVG | 3282 | | |
| | CCTGTGGGGGAAACAATGTTGGAAGTAAAACTGTGCACTGGTATCAGCAGAGGCCAGGCCA | | SKTVHWYQQRPGQAPVLVVYDNDARPSG | | | |
| | GGCCCCTGTGCTGGTCGTCTATGATAATGACGCCCGGCCCTCAGGGATTCCTGAGCGATTC | | IPERFSAYDSGNTATLTITRVEAGDEAD | | | |
| | TCTGCCTACGATTCTGGGAACACGGCCACCCTGACCATCACCAGGGTCGAGGCCGGGGATG | | YYCQVWDSGSDHYVFGTGTKVTVL | | | |
| | AGGCCGACTATTACTGTCAGGTGTGGGATAGTGGCAGTGATCATTATGTCTTCGGAACTGG | | | | | |
| | GACCAAGGTCACCGTCCTA | | | | | |
| ADI-75755 | GACATCCAGATGACCCAGTCTCCATCCTCCCTGTCTGCATCTGTAGGAGACAGAGTCACCA | 3000 | DIQMTQSPSSLSASVGDRVTITCRASQS | 3283 | | |
| | TCACTTGCCGGGCAAGTCAGAGCATTAGGAACTATTTAAATTGGTATCAGCAGAAACCAGG | | IRNYLNWYQQKPGKAPKLLIYDASSLQR | | | |
| | GAAAGCCCCTAAACTCCTGATCTATGATGCATCCAGTCTCCAACGTGGGGTCCCATCGAGG | | GVPSRFSGSGSGTDYTLTISSLQPEDFA | | | |
| | TTCAGTGGCAGTGGATCTGGGACAGATTACACTCTCACCATCAGCAGTCTGCAACCTGAAG | | TYYCQQSYNTPPWTFGQGTKVEIK | | | |
| | ATTTTGCAACTTACTACTGTCAACAGAGTTACAATACTCCTCCGTGGACGTTCGGCCAAGG | | | | | |
| | GACCAAGGTGGAAATCAAA | | | | | |
| ADI-75702 | GACATCCAGATGACCCAGTCTCCATCTTCCGTGTCTGCATCTGTAGGAGATAGAGTCACCA | 3001 | DIQMTQSPSSVSASVGDRVTITCRASQG | 3284 | | |
| | TCACTTGTCGGGCGAGTCAGGGTATTAGCAGGTGGTTAGCCTGGTATCAGCAGAAACCAGG | | ISRWLAWYQQKPGKAPKLLIYAASSLQS | | | |
| | GAAAGCCCCCAAGCTCCTGATCTATGCTGCATCCAGTTTGCAAAGTGGGGTCCCATCAAGG | | GVPSRFSGSGSGTDFTLTISSLQPEDFA | | | |
| | TTCAGCGGCAGTGGATCTGGGACAGATTTCACTCTCACCATCAGCAGCCTGCAGCCTGAAG | | TYYCQQADSFPLTFGGGTKVEIK | | | |
| | ATTTTGCAACTTACTATTGTCAACAGGCTGACAGTTTCCCCCTCACTTTCGGCGGAGGGAC | | | | | |
| | CAAGGTGGAAATCAAA | | | | | |
| ADI-75778 | GACATCCAGATGACCCAGTCTCCATCCTCCCTGTCTGCATCTGTAGGAGACAGAGTCACCA | 3000 | DIQMTQSPSSLSASVGDRVTITCRASQS | 3283 | | |
| | TCACTTGCCGGGCAAGTCAGAGCATTAGGAACTATTTAAATTGGTATCAGCAGAAACCAGG | | IRNYLNWYQQKPGKAPKLLIYDASSLQR | | | |
| | GAAAGCCCCTAAACTCCTGATCTATGATGCATCCAGTCTCCAACGTGGGGTCCCATCGAGG | | GVPSRFSGSGSGTDYTLTISSLQPEDFA | | | |
| | TTCAGTGGCAGTGGATCTGGGACAGATTACACTCTCACCATCAGCAGTCTGCAACCTGAAG | | TYYCQQSYNTPPWTFGQGTKVEIK | | | |
| | ATTTTGCAACTTACTACTGTCAACAGAGTTACAATACTCCTCCGTGGACGTTCGGCCAAGG | | | | | |
| | GACCAAGGTGGAAATCAAA | | | | | |
| ADI-75575 | GACATCCAGTTGACCCAGTCTCCATCCTCCCTGTCTGCATCTGTAGGAGACAGAGTCACCA | 3002 | DIQLTQSPSSLSASVGDRVTITCQASQD | 3285 | | |
| | TCACTTGCCAGGCGAGTCAGGACATTACCAAAAATTTAAATTGGTATCAGCAGAGACCAGG | | ITKNLNWYQQRPGKAPKLLIFDASYLET | | | |
| | GAAGGCCCCTAAGCTCCTGATCTTCGATGCGTCCTATTTGGAAACAGGGGTCCCGTCAAGG | | GVPSRFSGSGSGTDFTITISSLQPEDFA | | | |
| | TTCAGTGGAAGTGGATCTGGGACAGATTTTACTATCACCATCAGCAGCCTGCAGCCTGAAG | | TYFCQQYESLPPTFGGGTKVDIK | | | |
| | ATTTTGCAACATATTTCTGTCAACAATATGAAAGTCTCCCTCCTACTTTCGGCGGAGGGAC | | | | | |
| | CAAAGTGGATATCAAA | | | | | |
| ADI-75690 | CAGCCTGTGCTGACTCAGCCCCCTTCCCATTCTGCATCTTCTGGAACATCAGTCAGACTCA | 3003 | QPVLTQPPSHSASSGTSVRLTCMLSSGF | 3286 | | |
| | CCTGCATGCTGAGCAGTGGCTTCAGTGTTGGGGACTTCTGGATAAGGTGGTACCAACAAAA | | SVGDFWIRWYQQKPGNPPRYLLYYHSDS | | | |
| | GCCAGGGAACCCTCCCCGGTATCTCCTGTACTACCACTCAGACTCCAATAAGGGCCAAGGC | | NKGQGSGVPSRFSGSNDASANAGILRIS | | | |
| | TCTGGAGTTCCCAGCCGCTTCTCTGGATCCAACGATGCATCAGCCAATGCAGGGATTCTGC | | GLQPEDEADYYCGTWHSNSKTQVFGGGT | | | |
| | GTATCTCTGGGCTCCAGCCTGAGGATGAGGCTGACTATTACTGTGGTACATGGCACAGCAA | | KLTVL | | | |
| | CTCTAAGACTCAGGTGTTCGGCGGAGGGACCAAGCTCACCGTCCTA | | | | | |
| ADI-75735 | GACATCCAGATGACCCAGTCTCCATCCTCCCTGTCTGCATCTGTAGGAGACAGAGTCACCA | 3004 | DIQMTQSPSSLSASVGDRVTITCQASQH | 3287 | | |
| | TCACTTGCCAGGCGAGTCAGCACATTAGCAACTATTTAAATTGGTATCAGCACAGACCAGG | | ISNYLNWYQHRPGKAPKLLIYDASNLET | | | |
| | GAAAGCCCCTAAGCTCCTGATCTACGATGCATCCAATTTGGAAACAGGGGTCCCATCAAGG | | GVPSRFSGSGSGTDFTFTISSLQPEDIA | | | |
| | TTCAGTGGAAGTGGATCTGGGACAGATTTCACTTTCACGATCAGCAGCCTGCAGCCGGAAG | | TYYCQQYGSLPITFGQGTRLEIK | | | |
| | ATATTGCAACATATTATTGTCAGCAATACGGTAGTCTCCCGATCACCTTCGGCCAAGGGAC | | | | | |
| | ACGACTGGAGATTAAA | | | | | |
| ADI-75859 | GAAACGACACTCACGCAGTCTCCATCCTCCCTGTCTGCATCTGTGGGAGACAGAGTCACCA | 3005 | ETTLTQSPSSLSASVGDRVTITCRASQG | 3288 | | |
| | TCACTTGCCGGGCGAGTCAGGGCATTAGCAATTCTTTAGCCTGGTATCAGCAGAAACCAGG | | ISNSLAWYQQKPGKAPKLLLYAASTLES | | | |
| | GAAAGCCCCTAAACTCCTGCTCTATGCTGCATCCACATTGGAAAGTGGGGTCCCATCCAGG | | GVPSRFSGSGSGTDFTLTIASLQPEDFA | | | |
| | TTCAGTGGCAGTGGATCTGGGACGGATTTCACTCTCACCATCGCCAGCCTGCAGCCTGAAG | | TYYCQQYYSSVRFTFGQGTILDLN | | | |
| | ATTTTGCAACATATTACTGTCAACAGTATTATAGTTCCGTCCGGTTCACTTTTGGCCAGGG | | | | | |
| | GACCATTCTGGATCTCAAC | | | | | |
| ADI-75857 | GACATCCCGATGACCCAGTCTCCATCGACCCTGTCTGCATCTGTAGGAGACAGAGTCACCA | 3006 | DIPMTQSPSTLSASVGDRVTITCRASQG | 3289 | | |
| | TAACTTGCCGGGCGAGCCAGGGCATCAGCAATTCTTTAGCCTGGTATCAGCAGAAACCAGG | | ISNSLAWYQQKPGRAPKLLLYAASTLES | | | |
| | GAGAGCCCCTAAGCTCCTGCTCTATGCTGCATCCACATTGGAAAGTGGGGTCCCATCCAGG | | GVPSRFSGSGSGTDFTLTISSLQPEDFA | | | |
| | TTCAGTGGCAGTGGATCTGGGACGGATTTCACCCTCACCATCAGCAGCCTGCAGCCTGAAG | | TYYCQQYYSTPPRTFGQGTKVEIK | | | |
| | ATTTTGCAACTTATTACTGTCAACAGTATTATAGTACCCCCCCACGGACGTTCGGCCAAGG | | | | | |
| | GACCAAGGTGGAAATCAAA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75596 | IGKV1D- | DIQLTQ | 1876 | RASQTIN | 2087 | WYQHKP | 2282 | AASTLQ | 2417 | GVPSRFSG | 2560 | QQSYN | 2740 | FGQGTK | 2937 |
| | 39 | SPSSLS | | SFVN | | GKAPEL | | R | | SGTGTDFT | | SPRT | | VEIK | |
| | | ASVGDR | | | | LIY | | | | LTISSLQP | | | | | |
| | | VTITC | | | | | | | | EDFATYFC | | | | | |
| ADI-75838 | IGKV1D- | DIQMTQ | 1907 | RASQSIN | 2088 | WYQQKP | 2257 | VASSLQ | 2418 | GVPSRFSG | 2534 | QQSYS | 2741 | FGQGTK | 2937 |
| | 39 | SPSSLS | | SYLN | | GKAPKL | | S | | SGSGTDFT | | TPPWT | | VEIK | |
| | | ASIGDR | | | | LIY | | | | LTISSLQP | | | | | |
| | | VTITC | | | | | | | | EDFATYYC | | | | | |
| ADI-75607 | IGLV3-21 | SYVLTQ | 1883 | KGDDIGS | 2089 | WYQQKP | 2283 | DDTDRP | 2419 | GIPERFSG | 2561 | QVWDS | 2742 | FGGGTK | 2939 |
| | | PPSVSV | | KNVH | | GQAPVL | | S | | SNSGNPAT | | NIDHP | | LTVL | |
| | | APGQTA | | | | VVY | | | | LTISRVEA | | WV | | | |
| | | RITC | | | | | | | | GDEADYYC | | | | | |
| ADI-75570 | IGKV1-12 | DIQMTQ | 1885 | RASQGLS | 2059 | WYQQKP | 2257 | AASSLQ | 2391 | GVPSRFSG | 2534 | QQANS | 2710 | FGGGTK | 2936 |
| | | SPSSVS | | RWLA | | GKAPKL | | S | | SGSGTDFT | | FPLT | | VEIK | |
| | | ASVGDR | | | | LIY | | | | LTISSLQP | | | | | |
| | | VTITC | | | | | | | | EDFATYYC | | | | | |
| ADI-75833 | IGKV1D- | DIQMTQ | 1908 | RASQSIS | 2090 | WYRQKP | 2284 | AASRLQ | 2406 | GVPSRFSG | 2534 | QQSYS | 2743 | FGQGTR | 2940 |
| | 39 | SPSSLS | | SFLN | | GKAPEL | | S | | SGSGTDFT | | MPPIT | | LEIK | |
| | | TSVGDR | | | | LIY | | | | LTISSLQP | | | | | |
| | | VTITC | | | | | | | | EDFATYYC | | | | | |
| ADI-75845 | IGKV1- | DIQMTQ | 1878 | PASQGIS | 2091 | WYQQKP | 2259 | ATSTLE | 2420 | GVPSRFSG | 2562 | QQYYS | 2744 | FGQGTK | 2937 |
| | NL1 | SPSSLS | | NSLA | | GKAPKL | | S | | GGSGTDYT | | FRT | | VEIK | |
| | | ASVGDR | | | | LLY | | | | LTISGLQP | | | | | |
| | | VTITC | | | | | | | | EDFATYYC | | | | | |
| ADI-75606 | IGLV3-25 | TYMLTQ | 1909 | SGDAFPN | 2092 | WYQLKP | 2285 | KDSERP | 2400 | GIPERFSG | 2563 | QSADS | 2745 | FGGGTK | 2947 |
| | | PPSVSV | | QYAY | | GQAPVA | | S | | SSSGTTAT | | RGTV | | VTVL | |
| | | SPGQTA | | | | VIY | | | | LTINEVQA | | | | | |
| | | RITC | | | | | | | | EDEADYYC | | | | | |
| ADI-75568 | IGKV1- | EIPMTQ | 1910 | RASQGIR | 2076 | WYQQKL | 2286 | AASTLE | 2398 | GVPSRFSG | 2564 | QQYYD | 2746 | FGQGTK | 2944 |
| | NL1 | SPSYLS | | NSLA | | GKAPKL | | S | | SGSGTDYT | | IRT | | VDIK | |
| | | ASVGDR | | | | LLY | | | | LTISSLQP | | | | | |
| | | VTITC | | | | | | | | EDFATYFC | | | | | |
| ADI-75633 | IGLV3-21 | SYELTQ | 1911 | GGDSLGS | 2093 | WYQQKP | 2287 | DDSDRP | 2399 | GIPERFSG | 2565 | QVWDS | 2747 | FGGGTQ | 2948 |
| | | PPSVSV | | KNVH | | GQAPVL | | S | | SNSGNTAT | | NIGHP | | LTVL | |
| | | APGQTA | | | | VVF | | | | LTISRVEA | | WV | | | |
| | | RLTC | | | | | | | | GDEADYWC | | | | | |
| ADI-75617 | IGKV1D- | DIQLTQ | 1876 | RASQTVG | 2094 | WYQQKP | 2288 | TAPEME | 2421 | GVPPRFSG | 2566 | QQSHT | 2748 | FGQGTK | 2937 |
| | 39 | SPSSLS | | AFLN | | GKAPKL | | G | | TGSGTDFT | | IPYT | | VEIK | |
| | | ASVGDR | | | | LVF | | | | LTISSLQP | | | | | |
| | | VTITC | | | | | | | | EDFATYFC | | | | | |
| ADI-75579 | IGKV1D- | EIPMTQ | 1912 | RASQSIG | 2095 | WYRQKP | 2289 | AASTLQ | 2417 | GVPSRFSG | 2567 | QQSYT | 2749 | FGQGTK | 2937 |
| | 39 | SPSSLS | | SFLN | | GKAPNL | | R | | SGSGTDFT | | THMYT | | VEIK | |
| | | ASVGDR | | | | LIY | | | | LTISSLQP | | | | | |
| | | VTITC | | | | | | | | EDFATYFC | | | | | |
| ADI-75807 | IGKV4-1 | DIQLTQ | 1913 | KSSQSVV | 2096 | WYQQKP | 2290 | WASTRE | 2422 | GVPARFSG | 2568 | QQYYI | 2750 | FGGGTK | 2938 |
| | | SPDSLA | | YSSNNKN | | GQPPKL | | S | | SGSGTDFT | | TPLT | | VDIK | |
| | | VSLGER | | YLA | | LIY | | | | LTISSLQA | | | | | |
| | | ATITC | | | | | | | | EDVAVYYC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75596 | GACATCCAGTTGACCCAGTCTCCATCCTCCCTGTCTGCATCTGTAGGAGATAGAGTCACCA | 3007 | DIQLTQSPSSLSASVGDRVTITCRASQT | 3290 | | |
| | TCACTTGCCGGGCAAGTCAGACCATTAACAGCTTTGTTAATTGGTATCAGCACAAGCCAGG | | INSFVNWYQHKPGKAPELLIYAASTLQR | | | |
| | GAAAGCCCCTGAGCTCCTGATCTATGCTGCATCCACTTTGCAAAGGGGGGTCCCATCAAGG | | GVPSRFSGSGTGTDFTLTISSLQPEDFA | | | |
| | TTCAGTGGCAGTGGAACTGGGACAGATTTCACTCTCACCATCAGCAGTCTGCAACCTGAAG | | TYFCQQSYNSPRTFGQGTKVEIK | | | |
| | ATTTTGCAACGTACTTCTGTCAACAGAGTTACAATTCCCCTAGGACGTTCGGCCAAGGGAC | | | | | |
| | CAAGGTGGAGATCAAA | | | | | |
| ADI-75838 | GACATCCAGATGACCCAGTCTCCATCCTCCCTGTCTGCATCTGTGGGAGACAGAGTCACCA | 3008 | DIQMTQSPSSLSASIGDRVTITCRASQS | 3291 | | |
| | TCACTTGCCCGGCGAGTCAGGGCATTAGCAATTCTTTAGCCTGGTATCAGCAAAAACCAGG | | INSYLNWYQQKPGKAPKLLIYVASSLQS | | | |
| | GAAAGCCCCTAAGCTCCTGCTCTATGCTACATCCACATTGGAAAGTGGGGTCCCATCCAGG | | GVPSRFSGSGSGTDFTLTISSLQPEDFA | | | |
| | TTCAGTGGCGGTGGATCTGGGACGGATTACACTCTCACCATCAGCGGCCTGCAGCCTGAAG | | TYYCQQSYSTPPWTFGQGTKVEIK | | | |
| | ATTTTGCAACTTATTACTGTCAACAGTATTATAGTTTTCGGACGTTCGGCCAAGGGACCAA | | | | | |
| | GGTGGAAATCAAA | | | | | |
| ADI-75607 | TCGTATGTGCTGACTCAGCCACCCTCGGTGTCAGTGGCCCCAGGACAGACGGCCAGGATTA | 3009 | SYVLTQPPSVSVAPGQTARITCKGDDIG | 3292 | | |
| | CCTGTAAGGGAGACGACATTGGAAGTAAAAATGTGCACTGGTACCAGCAGAAGCCAGGCCA | | SKNVHWYQQKPGQAPVLVVYDDTDRPSG | | | |
| | GGCCCCTGTGCTGGTCGTCTATGATGATACCGACCGGCCCTCAGGGATCCCTGAGCGATTC | | IPERFSGSNSGNPATLTISRVEAGDEAD | | | |
| | TCTGGCTCCAACTCTGGGAACCCGGCCACCCTGACCATCAGCAGGGTCGAAGCCGGGGATG | | YYCQVWDSNIDHPWVFGGGTKLTVL | | | |
| | AGGCCGACTATTACTGTCAGGTATGGGATAGTAATATTGATCATCCTTGGGTGTTCGGCGG | | | | | |
| | AGGGACCAAGCTCACCGTCCTA | | | | | |
| ADI-75570 | GACATCCAGATGACCCAGTCTCCATCTTCCGTGTCTGCATCTGTAGGAGACAGAGTCACAA | 3010 | DIQMTQSPSSVSASVGDRVTITCRASQG | 3256 | | |
| | TCACTTGTCGGGCGAGCCAGGGTCTTAGCAGATGGTTAGCCTGGTATCAGCAGAAACCAGG | | LSRWLAWYQQKPGKAPKLLIYAASSLQS | | | |
| | GAAAGCCCCTAAACTCCTGATCTATGCTGCATCTAGTTTGCAGAGTGGGGTCCCATCAAGG | | GVPSRFSGSGSGTDFTLTISSLQPEDFA | | | |
| | TTCAGCGGCAGTGGATCTGGGACAGATTTCACTCTCACCATCAGCAGCCTGCAGCCTGAAG | | TYYCQQANSFPLTFGGGTKVEIK | | | |
| | ACTTTGCAACTTACTATTGTCAACAGGCTAACAGTTTCCCACTCACTTTCGGCGGAGGGAC | | | | | |
| | CAAGGTGGAGATCAAA | | | | | |
| ADI-75833 | GACATCCAGATGACCCAGTCTCCATCCTCCCTGTCTACATCTGTGGGAGACAGAGTCACCA | 3011 | DIQMTQSPSSLSTSVGDRVTITCRASQS | 3293 | | |
| | TCACTTGTCGGGCAAGTCAGAGCATTAGTAGTTTTTTAAATTGGTATCGGCAGAAACCAGG | | ISSFLNWYRQKPGKAPELLIYAASRLQS | | | |
| | GAAAGCCCCTGAACTCCTGATCTATGCTGCATCCAGGTTGCAAAGTGGGGTCCCATCAAGG | | GVPSRFSGSGSGTDFTLTISSLQPEDFA | | | |
| | TTCAGTGGCAGTGGATCTGGGACAGATTTCACTCTCACCATCAGCAGTCTGCAACCCGAAG | | TYYCQQSYSMPPITFGQGTRLEIK | | | |
| | ATTTTGCAACTTACTACTGTCAACAGAGTTACAGTATGCCTCCGATCACCTTCGGCCAAGG | | | | | |
| | GACACGACTGGAGATTAAA | | | | | |
| ADI-75845 | GACATCCAGATGACCCAGTCTCCATCCTCCCTGTCTGCATCTGTGGGAGACAGAGTCACCA | 3012 | DIQMTQSPSSLSASVGDRVTITCPASQG | 3294 | | |
| | TCACTTGCCCGGCGAGTCAGGGCATTAGCAATTCTTTAGCCTGGTATCAGCAAAAACCAGG | | ISNSLAWYQQKPGKAPKLLLYATSTLES | | | |
| | GAAAGCCCCTAAGCTCCTGCTCTATGCTACATCCACATTGGAAAGTGGGGTCCCATCCAGG | | GVPSRFSGGGSGTDYTLTISGLQPEDFA | | | |
| | TTCAGTGGCGGTGGATCTGGGACGGATTACACTCTCACCATCAGCGGCCTGCAGCCTGAAG | | TYYCQQYYSFRTFGQGTKVEIK | | | |
| | ATTTTGCAACTTATTACTGTCAACAGTATTATAGTTTTCGGACGTTCGGCCAAGGGACCAA | | | | | |
| | GGTGGAAATCAAA | | | | | |
| ADI-75606 | ACGTATATGCTGACTCAGCCCCCCTCGGTGTCAGTGTCCCCAGGACAGACGGCCAGGATCA | 3013 | TYMLTQPPSVSVSPGQTARITCSGDAFP | 3295 | | |
| | CCTGCTCTGGAGATGCATTCCCAAACCAATATGCTTATTGGTACCAACTGAAGCCAGGCCA | | NQYAYWYQLKPGQAPVAVIYKDSERPSG | | | |
| | GGCCCCTGTAGCGGTGATATATAAAGACAGTGAGAGGCCCTCAGGGATCCCTGAGCGATTC | | IPERFSGSSSGTTATLTINEVQAEDEAD | | | |
| | TCTGGCTCCAGCTCAGGGACAACAGCCACGTTGACCATCAATGAAGTCCAGGCAGAGGACG | | YYCQSADSRGTVFGGGTKVTVL | | | |
| | AGGCTGACTATTACTGTCAATCAGCAGACAGCCGGGGGACGGTGTTCGGCGGAGGGACCAA | | | | | |
| | GGTCACCGTCCTA | | | | | |
| ADI-75568 | GAAATCCCGATGACCCAGTCTCCATCCTACCTGTCTGCATCTGTAGGAGACAGAGTCACCA | 3014 | EIPMTQSPSYLSASVGDRVTITCRASQG | 3296 | | |
| | TCACTTGCCGGGCGAGTCAGGGCATTAGGAACTCTTTAGCCTGGTATCAGCAGAAATTAGG | | IRNSLAWYQQKLGKAPKLLLYAASTLES | | | |
| | GAAAGCCCCTAAGCTCCTGCTCTATGCTGCATCCACATTGGAAAGTGGGGTCCCATCCAGG | | GVPSRFSGSGSGTDYTLTISSLQPEDFA | | | |
| | TTCAGTGGCAGTGGATCTGGGACGGATTACACTCTCACCATCAGCAGCCTGCAGCCTGAAG | | TYFCQQYYDIRTFGQGTKVDIK | | | |
| | ATTTCGCAACTTATTTCTGTCAACAGTATTATGATATTCGGACGTTCGGCCAAGGGACCAA | | | | | |
| | GGTGGATATCAAA | | | | | |
| ADI-75633 | TCGTATGAGCTGACTCAGCCACCCTCGGTGTCCGTGGCCCCAGGACAGACGGCCAGACTTA | 3015 | SYELTQPPSVSVAPGQTARLTCGGDSLG | 3297 | | |
| | CCTGTGGGGGAGACAGCCTTGGAAGTAAAAATGTGCACTGGTACCAGCAGAAGCCAGGCCA | | SKNVHWYQQKPGQAPVLVVFDDSDRPSG | | | |
| | GGCCCCTGTGTTGGTCGTCTTTGATGATAGCGACCGGCCCTCAGGGATCCCTGAGCGATTC | | IPERFSGSNSGNTATLTISRVEAGDEAD | | | |
| | TCTGGCTCCAACTCTGGGAACACGGCCACCCTGACCATCAGCAGGGTCGAAGCCGGGGATG | | YWCQVWDSNIGHPWVFGGGTQLTVL | | | |
| | AGGCCGACTATTGGTGTCAGGTGTGGGATAGTAATATTGGTCATCCTTGGGTGTTCGGCGG | | | | | |
| | AGGCACCCAGCTGACCGTCCTA | | | | | |
| ADI-75617 | GACATCCAGTTGACGCAGTCTCCGTCCTCCCTGTCTGCTTCTGTGGGAGACAGAGTCACCA | 3016 | DIQLTQSPSSLSASVGDRVTITCRASQT | 3298 | | |
| | TCACTTGCCGGGCAAGTCAGACCGTTGGGGCCTTTTTAAATTGGTATCAGCAGAAACCAGG | | VGAFLNWYQQKPGKAPKLLVFTAPEMEG | | | |
| | GAAAGCCCCTAAGCTCCTGGTCTTTACTGCACCCGAGATGGAAGGCGGAGTCCCCCCGAGG | | GVPPRFSGTGSGTDFTLTISSLQPEDFA | | | |
| | TTCAGTGGCACTGGATCTGGCACAGATTTCACTCTCACCATCAGCAGTCTACAACCTGAAG | | TYFCQQSHTIPYTFGQGTKVEIK | | | |
| | ACTTTGCAACTTACTTCTGTCAACAGAGTCACACTATCCCATACACTTTTGGCCAGGGGAC | | | | | |
| | CAAGGTGGAGATCAAA | | | | | |
| ADI-75579 | GAAATCCCGATGACCCAGTCTCCATCCTCCCTGTCTGCATCTGTAGGAGACAGAGTCACCA | 3017 | EIPMTQSPSSLSASVGDRVTITCRASQS | 3299 | | |
| | TCACTTGCCGGGCAAGTCAGAGCATTGGCAGCTTTTTAAATTGGTATCGCCAGAAACCAGG | | IGSFLNWYRQKPGKAPNLLIYAASTLQR | | | |
| | GAAAGCCCCGAACCTCCTGATCTATGCTGCGTCCACTTTGCAAAGGGGAGTCCCTTCAAGG | | GVPSRFSGSGSGTDFTLTISSLQPEDFA | | | |
| | TTCAGTGGCAGTGGATCTGGGACAGATTTCACTCTCACCATCAGCAGTCTGCAACCTGAGG | | TYFCQQSYTTHMYTFGQGTKVEIK | | | |
| | ATTTTGCAACTTACTTTTGTCAACAGAGTTACACTACTCATATGTACACTTTTGGCCAGGG | | | | | |
| | GACCAAGGTGGAGATCAAA | | | | | |
| ADI-75807 | GACATCCAGTTGACCCAGTCTCCAGACTCCCTGGCTGTGTCTCTGGGCGAGAGGGCCACCA | 3018 | DIQLTQSPDSLAVSLGERATITCKSSQS | 3300 | | |
| | TCACCTGCAAGTCCAGCCAGAGTGTTGTATACAGCTCCAACAATAAGAACTACTTAGCTTG | | VVYSSNNKNYLAWYQQKPGQPPKLLIYW | | | |
| | GTACCAGCAAAAACCAGGACAGCCTCCTAAGCTGCTCATTTACTGGGCATCTACCCGGGAA | | ASTRESGVPARFSGSGSGTDFTLTISSL | | | |
| | TCCGGGGTCCCTGCCCGATTCAGTGGCAGCGGGTCTGGGACAGATTTCACTCTCACCATCA | | QAEDVAVYYCQQYYITPLTFGGGTKVDI | | | |
| | GCAGCCTGCAGGCTGAAGATGTGGCAGTTTATTACTGTCAGCAATATTATATTACTCCTCT | | K | | | |
| | CACTTTCGGCGGAGGGACCAAAGTGGATATCAAA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75780 | IGKV1D- | DIQMTQ | 1896 | RASQSMT | 2097 | WYQQRP | 2278 | GASSLH | 2404 | GVPSRFSG | 2534 | QQSYS | 2751 | FGQGTK | 2937 |
| | 39 | SPSSLS | | SSLN | | GKAPKL | | S | | SGSGTDFT | | TPWT | | VEIK | |
| | | ASVGDR | | | | LIF | | | | LTISSLQP | | | | | |
| | | VTIAC | | | | | | | | EDFATYYC | | | | | |
| ADI-75748 | IGKV1D- | EMTLTQ | 1914 | RASHRID | 2098 | WYQQKP | 2291 | VASTLQ | 2423 | GVPSRFTG | 2551 | QQSYT | 2752 | FGPGTK | 2942 |
| | 39 | SPSSLS | | RYLN | | GQAPKL | | S | | SGSGTDFT | | TPRT | | VEIK | |
| | | ASVGDR | | | | LIY | | | | LTISSLQP | | | | | |
| | | VTITC | | | | | | | | EDFATYYC | | | | | |
| ADI-75820 | IGLV3-25 | SYELTQ | 1915 | CGDALPN | 2099 | WYQQKP | 2292 | KDSERP | 2400 | GIPERFSG | 2546 | QSADS | 2753 | FGGGTK | 2947 |
| | | PPSVSV | | QYTY | | GQAPVV | | S | | STSGTTVT | | SGVV | | VTVL | |
| | | SPGQTA | | | | IIY | | | | LTISGVQA | | | | | |
| | | RITC | | | | | | | | EDEADYYC | | | | | |
| ADI-75612 | IGKV3D- | VSPGER | 1916 | RASQSVS | 2100 | WYQQKP | 2293 | DASTRA | 2424 | GIPARFSG | 2569 | QQYNN | 2754 | FGQGTK | 2937 |
| | 15 | EMTLTQ | | SSLA | | GQPPRL | | T | | SGSGTEFT | | WPTWT | | VEIK | |
| | | SPATLS | | | | LIY | | | | LTISSLQS | | | | | |
| | | ATLSC | | | | | | | | EDFAVYYC | | | | | |
| ADI-75696 | IGKV1-12 | DIQMTQ | 1885 | RASQDIG | 2101 | WYQQKP | 2294 | AAASLQ | 2425 | GVPSRFSG | 2570 | QQATS | 2755 | FGGGTK | 2936 |
| | | SPSSVS | | SWLA | | GKAPRL | | S | | SGSGTEFT | | FPLT | | VEIK | |
| | | ASVGDR | | | | LIY | | | | LTISRLQP | | | | | |
| | | VTITC | | | | | | | | EDFATYYC | | | | | |
| ADI-75571 | IGKV1- | EIQMTQ | 1881 | RASQGIS | 2062 | WYQQRR | 2295 | AASTLE | 2398 | GVPSRFSG | 2534 | QQYYS | 2756 | FGGGTK | 2936 |
| | NL1 | SPSSLS | | NSLA | | GKAPKL | | S | | SGSGTDFT | | PPPLT | | VEIK | |
| | | ASVGDR | | | | LLY | | | | LTISSLQP | | | | | |
| | | VTITC | | | | | | | | EDFATYYC | | | | | |
| ADI-75766 | IGKV1D- | ASVGDR | 1917 | RASQAIS | 2102 | WYQQKP | 2257 | AASSLH | 2426 | GVPSRFSG | 2536 | QQSYT | 2757 | FGGGTK | 2936 |
| | 39 | EIQLTQ | | SYLN | | GKAPKL | | S | | SGSGTDFT | | IPTLT | | VEIK | |
| | | SPSSLS | | | | LIY | | | | LTINSLQP | | | | | |
| | | VTITC | | | | | | | | EDFATYYC | | | | | |
| ADI-75635 | IGKV1- | DIQMTQ | 1878 | RASQGIG | 2103 | WYQQKP | 2296 | AASTLE | 2398 | GVPSRFSG | 2534 | QQYYT | 2758 | FGQGTK | 2937 |
| | NL1 | SPSSLS | | NSLA | | GKAPKF | | S | | SGSGTDFT | | TRT | | VEIK | |
| | | ASVGDR | | | | LLY | | | | LTISSLQP | | | | | |
| | | VTITC | | | | | | | | EDFATYYC | | | | | |
| ADI-75864 | IGLV2-14 | QSVLTQ | 1918 | GVFNYVS | 2104 | WYQQHP | 2265 | EVSNRP | 2427 | GVSNRFSG | 2571 | SSYTT | 2759 | FGGGTQ | 2948 |
| | | PASVSG | | TATTSDV | | GKAPKL | | S | | SKSGNTAS | | SSTV | | LTVL | |
| | | SPGQSI | | | | MIY | | | | LTISGLQA | | | | | |
| | | TISC | | | | | | | | EDEADYFC | | | | | |
| ADI-75877 | IGLV3-21 | SYELTQ | 1919 | GGNNIGS | 2105 | WYQQKP | 2283 | EDSDRP | 2428 | GIPERFSG | 2572 | QVWDG | 2760 | FGTGTK | 2949 |
| | | PPSVSV | | ESVH | | GQAPVL | | S | | STSGNTAT | | ISVHY | | LTVL | |
| | | APGQTA | | | | VVY | | | | LTISRVEA | | V | | | |
| | | RITC | | | | | | | | GDEADYYC | | | | | |
| ADI-75827 | IGKV3D- | EIAMTQ | 1877 | RASESVS | 2106 | WYQQKP | 2258 | GASTRA | 2383 | GIPARFSG | 2529 | QQYID | 2761 | FGQGTK | 2937 |
| | 15 | SPATLS | | SNLA | | GQAPRL | | T | | SGSGTEFT | | WPPGY | | VEIK | |
| | | VSPGER | | | | LIY | | | | LTISSLQS | | T | | | |
| | | ATLSC | | | | | | | | EDFAVYFC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75780 | GACATCCAGATGACCCAGTCTCCATCCTCCCTGTCTGCATCTGTAGGAGACAGAGTCACCA | 3019 | DIQMTQSPSSLSASVGDRVTIACRASQS | 3301 | | |
| | TCGCTTGCCGGGCAAGTCAGAGCATGACGAGCTCTTTAAATTGGTATCAGCAGAGACCAGG | | MTSSLNWYQQRPGKAPKLLIFGASSLHS | | | |
| | GAAAGCCCCTAAGCTCCTGATCTTTGGTGCATCCAGTTTGCATAGTGGGGTCCCATCAAGG | | GVPSRFSGSGSGTDFTLTISSLQPEDFA | | | |
| | TTCAGTGGCAGTGGATCTGGGACAGATTTCACTCTCACCATCAGCAGTCTGCAACCTGAAG | | TYYCQQSYSTPWTFGQGTKVEIK | | | |
| | ATTTTGCAACTTACTACTGTCAACAGAGTTACAGTACCCCGTGGACGTTCGGCCAAGGGAC | | | | | |
| | CAAGGTGGAAATCAAA | | | | | |
| ADI-75748 | GAAATGACACTCACGCAGTCTCCATCCTCCCTGTCTGCATCTGTAGGAGACAGAGTCACCA | 3020 | EMTLTQSPSSLSASVGDRVTITCRASHR | 3302 | | |
| | TCACTTGTCGGGCAAGTCACAGGATTGACAGGTATTTAAATTGGTATCAGCAGAAACCAGG | | IDRYLNWYQQKPGQAPKLLIYVASTLQS | | | |
| | GCAAGCCCCCAAGCTCCTGATCTATGTAGCATCCACTTTGCAAAGTGGGGTCCCATCAAGG | | GVPSRFTGSGSGTDFTLTISSLQPEDFA | | | |
| | TTCACTGGCAGTGGATCTGGGACAGATTTCACTCTCACTATCAGCAGTCTGCAACCTGAGG | | TYYCQQSYTTPRTFGPGTKVEIK | | | |
| | ATTTTGCAACTTACTACTGTCAACAGAGTTACACTACCCCTCGGACTTTCGGCCCTGGGAC | | | | | |
| | CAAGGTGGAGATCAAA | | | | | |
| ADI-75820 | TCGTATGAGCTGACTCAGCCACCCTCGGTGTCAGTGTCCCCAGGACAGACGGCCAGGATCA | 3021 | SYELTQPPSVSVSPGQTARITCCGDALP | 3303 | | |
| | CCTGCTGTGGAGATGCATTGCCAAACCAATATACTTATTGGTACCAGCAGAAGCCAGGCCA | | NQYTYWYQQKPGQAPVVIIYKDSERPSG | | | |
| | GGCCCCTGTGGTGATAATATATAAAGACAGTGAGAGGCCCTCAGGGATCCCTGAGCGATTC | | IPERFSGSTSGTTVTLTISGVQAEDEAD | | | |
| | TCTGGCTCCACCTCAGGGACAACAGTCACGTTGACCATCAGTGGAGTCCAGGCAGAAGACG | | YYCQSADSSGVVFGGGTKVTVL | | | |
| | AGGCTGACTATTACTGTCAATCAGCAGACAGCAGTGGTGTTGTATTCGGCGGAGGGACCAA | | | | | |
| | GGTCACCGTCCTA | | | | | |
| ADI-75612 | GAAATGACACTGACGCAGTCTCCAGCCACCCTGTCTGTGTCTCCAGGGGAAAGAGCCACCC | 3022 | EMTLTQSPATLSVSPGERATLSCRASQS | 3304 | | |
| | TCTCCTGCAGGGCCAGTCAGAGTGTCAGCAGCAGCTTAGCCTGGTACCAGCAGAAACCTGG | | VSSSLAWYQQKPGQPPRLLIYDASTRAT | | | |
| | CCAGCCTCCCAGGCTCCTCATCTATGATGCATCCACCAGGGCCACTGGTATCCCAGCCAGG | | GIPARFSGSGSGTEFTLTISSLQSEDFA | | | |
| | TTCAGTGGCAGTGGGTCTGGGACAGAGTTCACTCTCACCATCAGCAGCCTGCAGTCTGAAG | | VYYCQQYNNWPTWTFGQGTKVEIK | | | |
| | ATTTCGCAGTTTATTACTGTCAGCAGTATAATAACTGGCCTACGTGGACGTTCGGCCAAGG | | | | | |
| | GACCAAGGTGGAGATCAAA | | | | | |
| ADI-75696 | GACATCCAGATGACCCAGTCTCCATCTTCCGTGTCTGCATCTGTAGGAGACCGAGTCACCA | 3023 | DIQMTQSPSSVSASVGDRVTITCRASQD | 3305 | DIQMTQSPS | 3305 |
| | TCACTTGTCGGGCGAGTCAGGATATTGGCAGCTGGCTAGCCTGGTATCAGCAGAAACCAGG | | IGSWLAWYQQKPGKAPRLLIYAAASLQS | | SVSASVGDR | |
| | GAAAGCCCCTAGACTCCTGATCTATGCTGCAGCCAGTTTGCAAAGTGGGGTCCCATCAAGG | | GVPSRFSGSGSGTEFTLTISRLQPEDFA | | VTITCRASQ | |
| | TTCAGCGGCAGTGGCTCTGGGACAGAATTCACTCTCACCATCAGCAGGCTACAGCCTGAAG | | TYYCQQATSFPLTFGGGTKVEIK | | DIGSWLAWY | |
| | ATTTTGCAACTTACTATTGTCAACAGGCTACCAGTTTTCCGCTCACTTTCGGCGGAGGGAC | | | | QQKPGKAPR | |
| | CAAGGTGGAAATCAAA | | | | LLIYAAASL | |
| | | | | | QSGVPSRFS | |
| | | | | | GSGSGTEFT | |
| | | | | | LTISRLQPE | |
| | | | | | DFATYYCQQ | |
| | | | | | ATSFPLTFG | |
| | | | | | GGTKVEIK | |
| ADI-75571 | GAAATCCAGATGACCCAGTCTCCATCCTCCCTGTCTGCATCTGTTGGAGACAGAGTCACCA | 3024 | EIQMTQSPSSLSASVGDRVTITCRASQG | 3306 | | |
| | TCACTTGCCGGGCGAGTCAGGGCATTAGCAATTCCTTAGCCTGGTATCAACAGAGACGAGG | | ISNSLAWYQQRRGKAPKLLLYAASTLES | | | |
| | GAAAGCCCCTAAGCTCCTGCTCTATGCTGCATCCACTTTGGAAAGTGGGGTCCCATCCAGG | | GVPSRFSGSGSGTDFTLTISSLQPEDFA | | | |
| | TTCAGTGGCAGTGGATCTGGGACGGATTTCACTCTCACCATCAGCAGCCTCCAGCCTGAGG | | TYYCQQYYSPPPLTFGGGTKVEIK | | | |
| | ATTTTGCAACTTATTACTGTCAACAGTACTATAGTCCCCCTCCGCTCACTTTCGGCGGAGG | | | | | |
| | GACCAAGGTGGAAATCAAA | | | | | |
| ADI-75766 | GAAATCCAATTGACCCAGTCTCCATCCTCCCTGTCTGCATCTGTGGGAGACAGAGTCACCA | 3025 | EIQLTQSPSSLSASVGDRVTITCRASQA | 3307 | | |
| | TCACTTGCCGGGCAAGTCAGGCCATTAGCAGTTATTTAAATTGGTATCAGCAGAAACCAGG | | ISSYLNWYQQKPGKAPKLLIYAASSLHS | | | |
| | GAAAGCCCCTAAGCTCCTCATCTATGCTGCATCCAGTTTGCACAGTGGGGTCCCATCAAGG | | GVPSRFSGSGSGTDFTLTINSLQPEDFA | | | |
| | TTCAGTGGCAGTGGATCTGGGACAGATTTCACTCTCACCATCAACAGTCTGCAACCTGAAG | | TYYCQQSYTIPTLTFGGGTKVEIK | | | |
| | ATTTTGCAACTTACTACTGTCAACAGAGTTACACTATCCCTACGCTCACTTTCGGCGGAGG | | | | | |
| | GACCAAGGTGGAGATCAAA | | | | | |
| ADI-75635 | GACATCCAGATGACCCAGTCTCCATCCTCCCTGTCTGCATCTGTAGGAGACAGAGTCACCA | 3026 | DIQMTQSPSSLSASVGDRVTITCRASQG | 3308 | | |
| | TCACTTGCCGGGCGAGTCAGGGCATTGGCAATTCTTTAGCCTGGTATCAGCAGAAACCAGG | | IGNSLAWYQQKPGKAPKFLLYAASTLES | | | |
| | GAAAGCCCCTAAGTTCCTGCTCTATGCTGCATCCACATTGGAAAGTGGGGTCCCATCCAGG | | GVPSRFSGSGSGTDFTLTISSLQPEDFA | | | |
| | TTCAGTGGCAGTGGATCTGGGACGGATTTCACTCTCACCATCAGCAGCCTGCAACCTGAAG | | TYYCQQYYTTRTFGQGTKVEIK | | | |
| | ATTTTGCAACTTATTACTGTCAACAATACTATACTACCCGGACGTTCGGCCAAGGGACCAA | | | | | |
| | GGTGGAAATCAAA | | | | | |
| ADI-75864 | CAGTCTGTGCTGACTCAGCCTGCCTCCGTGTCTGGGTCTCCTGGACAGTCGATCACCATCT | 3027 | QSVLTQPASVSGSPGQSITISCTATTSD | 3309 | QSALTQPAS | 3526 |
| | CCTGCACTGCAACCACCAGTGACGTTGGTGTTTTTAACTATGTCTCGTGGTACCAACAGCA | | VGVFNYVSWYQQHPGKAPKLMIYEVSNR | | VSGSPGQSI | |
| | CCCAGGCAAGGCCCCCAAACTCATGATTTATGAGGTCAGTAATCGGCCCTCAGGGGTTTCT | | PSGVSNRFSGSKSGNTASLTISGLQAED | | TISCTATTS | |
| | AATCGCTTCTCTGGCTCCAAGTCTGGCAACACGGCCTCCCTGACCATCTCTGGGCTCCAGG | | EADYFCSSYTTSSTVFGGGTQLTVL | | DVGVFNYVS | |
| | CTGAGGACGAGGCTGATTATTTCTGCAGCTCATATACAACCAGCAGCACGGTGTTCGGCGG | | | | WYQQHPGKA | |
| | AGGGACCCAGCTGACCGTCCTA | | | | PKLMIYEVS | |
| | | | | | NRPSGVSNR | |
| | | | | | FSGSKSGNT | |
| | | | | | ASLTISGLQ | |
| | | | | | AEDEADYFC | |
| | | | | | SSYTTSSTV | |
| | | | | | FGGGTQLTVL | |
| ADI-75877 | TCGTATGAGCTGACTCAGCCACCCTCGGTGTCAGTGGCCCCAGGACAGACGGCCAGGATTA | 3028 | SYELTQPPSVSVAPGQTARITCGGNNIG | 3310 | | |
| | CCTGTGGGGGAAACAACATTGGAAGTGAAAGTGTCCACTGGTACCAGCAGAAGCCAGGCCA | | SESVHWYQQKPGQAPVLVVYEDSDRPSG | | | |
| | GGCCCCTGTGCTGGTCGTCTATGAGGACAGCGACCGGCCCTCAGGGATCCCTGAGCGATTC | | IPERFSGSTSGNTATLTISRVEAGDEAD | | | |
| | TCTGGCTCCACCTCTGGGAACACGGCCACCCTGACCATCAGCAGGGTCGAAGCCGGGGATG | | YYCQVWDGISVHYVFGTGTKLTVL | | | |
| | AGGCCGATTATTACTGTCAGGTGTGGGATGGTATTAGTGTTCATTATGTCTTCGGAACTGG | | | | | |
| | GACCAAGCTCACCGTCCTA | | | | | |
| ADI-75827 | GAAATTGCGATGACGCAGTCTCCAGCCACCCTGTCTGTGTCTCCAGGGGAAAGAGCCACCC | 3029 | EIAMTQSPATLSVSPGERATLSCRASES | 3311 | | |
| | TCTCCTGCAGGGCCAGTGAGAGTGTTAGCAGCAACTTAGCCTGGTACCAGCAGAAACCTGG | | VSSNLAWYQQKPGQAPRLLIYGASTRAT | | | |
| | CCAGGCTCCCAGACTCCTCATCTATGGTGCATCCACCAGGGCCACTGGTATCCCAGCCAGG | | GIPARFSGSGSGTEFTLTISSLQSEDFA | | | |
| | TTCAGTGGCAGTGGGTCTGGGACAGAGTTCACTCTCACCATCAGCAGCCTGCAGTCTGAAG | | VYFCQQYIDWPPGYTFGQGTKVEIK | | | |
| | ATTTTGCAGTTTATTTCTGTCAGCAGTATATTGACTGGCCTCCGGGGTACACTTTTGGCCA | | | | | |
| | GGGGACCAAGGTGGAGATCAA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75683 | IGKV1D- | EIQMTQ | 1920 | RASQSIS | 2050 | WYQQKP | 2257 | GASSLH | 2429 | GVPSRFSG | 2534 | QQSYT | 2752 | FGQGTK | 2944 |
| | 39 | SPSSLP | | SYLN | | GKAPKL | | A | | SGSGTDFT | | TPRT | | VDIK | |
| | | ASVGDR | | | | LIY | | | | LTISSLQP | | | | | |
| | | VTITC | | | | | | | | EDFATYYC | | | | | |
| ADI-75776 | IGKV1D- | DIQLTQ | 1876 | RASQRVS | 2107 | WFQQKP | 2297 | AASSLQ | 2391 | GVPSRFSG | 2573 | QQSYD | 2762 | FGQGTR | 2940 |
| | 39 | SPSSLS | | SYLN | | GKAPRL | | S | | SGSGTDFT | | AIT | | LEIK | |
| | | ASVGDR | | | | LIF | | | | LTISSLEP | | | | | |
| | | VTITC | | | | | | | | EDFATYYC | | | | | |
| ADI-75785 | IGKV1-12 | DITMTQ | 1921 | RASQSIG | 2108 | WYQQKP | 2257 | AASILQ | 2430 | GVPSRFSG | 2534 | QQADS | 2763 | FGPGTK | 2950 |
| | | SPSSVS | | SWLG | | GKAPKL | | S | | SGSGTDFT | | FPPA | | VDIK | |
| | | ASVGDR | | | | LIY | | | | LTISSLQP | | | | | |
| | | VSITC | | | | | | | | EDFATYYC | | | | | |
| ADI-75594 | IGKV1- | EIPMTQ | 1912 | RASQGIG | 2103 | WYQQKP | 2296 | AASTLE | 2398 | GVPSRFSG | 2534 | QQYYT | 2758 | FGQGTK | 2944 |
| | NL1 | SPSSLS | | NSLA | | GKAPKF | | S | | SGSGTDFT | | TRT | | VDIK | |
| | | ASVGDR | | | | LLY | | | | LTISSLQP | | | | | |
| | | VTITC | | | | | | | | EDFATYYC | | | | | |
| ADI-75801 | IGLV6-57 | NLMLTQ | 1922 | TRSSGSI | 2109 | WYQQRP | 2298 | EDNQRP | 2431 | GVPDRFSG | 2574 | QSYDT | 2764 | FGGGTK | 2939 |
| | | PHSVSE | | ASNYVQ | | GSAPTT | | S | | SIDSSSNS | | RLRGV | | LTVL | |
| | | SPGKTV | | | | VIY | | | | ASLTISGL | | | | | |
| | | TISC | | | | | | | | KTEDEADY | | | | | |
| | | | | | | | | | | YC | | | | | |
| ADI-75624 | IGKV2D- | VTPGEP | 1923 | KSSQSLL | 2110 | WYLQKP | 2260 | LGSNRA | 2387 | GVPDRFSG | 2575 | MQALQ | 2765 | FGQGTR | 2940 |
| | 28 | EIVMTQ | | HSNGYKY | | GQSPQL | | S | | SGSGTDFT | | TPIT | | LEIK | |
| | | SPLSLP | | LD | | LIY | | | | LKISRVEA | | | | | |
| | | ASISC | | | | | | | | EDVGIYYC | | | | | |
| ADI-75764 | IGKV1-33 | DIQLTQ | 1876 | QASQDIG | 2111 | WFQQKA | 2299 | EASNLE | 2393 | GVPSRFSG | 2576 | QQYDS | 2766 | FGPGTK | 2950 |
| | | SPSSLS | | NYLS | | GKAPRL | | T | | SGSGTDFA | | LLT | | VDIK | |
| | | ASVGDR | | | | LMY | | | | LTISSLQP | | | | | |
| | | VTITC | | | | | | | | EDIATYYC | | | | | |
| ADI-75806 | IGKV1D- | DIQLTQ | 1876 | RASQSVN | 2112 | WFQQKP | 2300 | AASSLQ | 2391 | GVPSRFSG | 2577 | QQSYA | 2767 | FGPGTK | 2942 |
| | 39 | SPSSLS | | NYLN | | GKAPKL | | S | | SGSGTDYT | | TPLT | | VEIK | |
| | | ASVGDR | | | | LIY | | | | LAISSLQP | | | | | |
| | | VTITC | | | | | | | | EDFATYYC | | | | | |
| ADI-75630 | IGKV1- | DIPLTQ | 1924 | RASQDIS | 2113 | WYQQKP | 2259 | TASTLE | 2432 | GVPSRFSG | 2531 | QQYYS | 2768 | FGGGTK | 2938 |
| | NL1 | SPSSLS | | NSLA | | GKAPKL | | S | | SGSGTDYT | | ALGRT | | VDIK | |
| | | ASVGDR | | | | LLY | | | | LTISSLQP | | | | | |
| | | VTITC | | | | | | | | EDFATYYC | | | | | |
| ADI-75609 | IGLV1-36 | QSVLTQ | 1925 | SGSSSNI | 2114 | WYQQLP | 2301 | YDDLLP | 2433 | GVSDRFSG | 2578 | VTFDD | 2769 | FGGGTK | 2939 |
| | | PPSVSA | | EINAVN | | GKAPKL | | S | | SKSGTSAS | | RLDGW | | LTVL | |
| | | APRQRV | | | | LIY | | | | LAISGLQS | | V | | | |
| | | TISC | | | | | | | | EDEADYYC | | | | | |
| ADI-75671 | IGKV1D- | EIKMTQ | 1926 | RASQSIT | 2115 | WYQQKP | 2257 | GASSLH | 2407 | GVPSRFSG | 2579 | QQSYT | 2752 | FGQGTK | 2937 |
| | 39 | SPSSLP | | SYLN | | GKAPKL | | T | | SGSGTHFT | | TPRT | | VEIK | |
| | | ASVGDR | | | | LIY | | | | LTISSLQP | | | | | |
| | | VTITC | | | | | | | | EDFATYFC | | | | | |
| ADI-75660 | IGKV1D- | EIQMTQ | 1881 | RASQGIS | 2116 | WYQQKP | 2257 | DASSLE | 2434 | GVPSRFSG | 2534 | QQFNH | 2770 | FGQGTR | 2940 |
| | 13 | SPSSLS | | FALA | | GKAPKL | | S | | SGSGTDFT | | YPIT | | LEIK | |
| | | ASVGDR | | | | LIY | | | | LTISSLQP | | | | | |
| | | VTITC | | | | | | | | EDFATYYC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75683 | GAAATCCAAATGACCCAGTCTCCATCCTCCCTGCCTGCATCTGTAGGAGACAGAGTCACCA | 3030 | EIQMTQSPSSLPASVGDRVTITCRASQS | 3312 | | |
| | TCACTTGCCGGGCAAGTCAGAGCATTAGCAGTTATTTAAATTGGTATCAGCAAAAACCAGG | | ISSYLNWYQQKPGKAPKLLIYGASSLHA | | | |
| | GAAAGCCCCTAAGCTCCTGATTTATGGTGCATCCAGTTTGCATGCTGGGGTCCCATCAAGG | | GVPSRFSGSGSGTDFTLTISSLQPEDFA | | | |
| | TTCAGTGGCAGTGGATCTGGGACAGATTTCACTCTCACCATCAGCAGTCTGCAACCTGAAG | | TYYCQQSYTTPRTFGQGTKVDIK | | | |
| | ATTTTGCAACTTACTACTGTCAACAGAGTTACACTACCCCTCGGACTTTTGGCCAGGGGAC | | | | | |
| | CAAGGTGGATATCAAA | | | | | |
| ADI-75776 | GACATCCAGTTGACCCAGTCTCCATCGTCCCTGTCTGCATCTGTAGGAGACAGAGTCACCA | 3031 | DIQLTQSPSSLSASVGDRVTITCRASQR | 3313 | | |
| | TCACTTGCCGGGCAAGTCAGAGAGTTAGCAGCTATTTAAATTGGTTTCAACAGAAACCAGG | | VSSYLNWFQQKPGKAPRLLIFAASSLQS | | | |
| | GAAAGCCCCTAGGCTCCTGATCTTTGCTGCATCCAGTTTGCAAAGTGGGGTCCCTTCAAGG | | GVPSRFSGSGSGTDFTLTISSLEPEDFA | | | |
| | TTCAGTGGCAGTGGATCTGGGACAGATTTCACTCTCACCATCAGCAGTCTGGAACCTGAAG | | TYYCQQSYDAITFGQGTRLEIK | | | |
| | ATTTTGCAACTTACTACTGTCAACAGAGTTATGATGCGATTACCTTCGGCCAAGGGACACG | | | | | |
| | ACTGGAGATTAAA | | | | | |
| ADI-75785 | GACATCACGATGACCCAGTCTCCATCTTCCGTGTCTGCATCTGTAGGAGACAGAGTCTCCA | 3032 | DITMTQSPSSVSASVGDRVSITCRASQS | 3314 | | |
| | TCACTTGTCGGGCGAGTCAAAGTATTGGCAGCTGGTTAGGCTGGTATCAGCAGAAACCAGG | | IGSWLGWYQQKPGKAPKLLIYAASILQS | | | |
| | GAAAGCCCCTAAACTCCTGATCTATGCTGCGTCCATTTTGCAAAGTGGGGTCCCATCAAGG | | GVPSRFSGSGSGTDFTLTISSLQPEDFA | | | |
| | TTCAGCGGCAGTGGATCTGGGACAGATTTCACTCTCACCATCAGTAGCCTGCAGCCTGAAG | | TYYCQQADSFPPAFGPGTKVDIK | | | |
| | ATTTTGCGACTTACTATTGTCAACAGGCTGACAGTTTCCCTCCGGCTTTCGGCCCTGGGAC | | | | | |
| | CAAAGTGGATATCAAA | | | | | |
| ADI-75594 | GAAATCCCGATGACCCAGTCTCCATCCTCCCTGTCTGCATCTGTAGGAGACAGAGTCACCA | 3033 | EIPMTQSPSSLSASVGDRVTITCRASQG | 3315 | | |
| | TCACTTGCCGGGCGAGTCAGGGCATTGGCAATTCTTTAGCCTGGTATCAGCAGAAACCGGG | | IGNSLAWYQQKPGKAPKELLYAASTLES | | | |
| | GAAAGCCCCTAAGTTCCTGCTCTATGCTGCATCCACATTGGAAAGTGGGGTCCCATCCAGG | | GVPSRFSGSGSGTDFTLTISSLQPEDFA | | | |
| | TTCAGTGGCAGTGGATCTGGGACGGATTTCACTCTCACCATCAGCAGCCTGCAACCTGAAG | | TYYCQQYYTTRTFGQGTKVDIK | | | |
| | ATTTTGCAACTTATTACTGTCAACAATACTATACTACCCGGACGTTCGGCCAAGGGACCAA | | | | | |
| | GGTGGATATCAAA | | | | | |
| ADI-75801 | AATCTTATGCTGACTCAGCCCCACTCTGTGTCGGAGTCTCCGGGGAAGACGGTAACCATCT | 3034 | NLMLTQPHSVSESPGKTVTISCTRSSGS | 3316 | | |
| | CCTGCACCCGCAGCAGTGGCAGCATTGCCAGCAACTATGTGCAGTGGTACCAGCAGCGCCC | | IASNYVQWYQQRPGSAPTTVIYEDNQRP | | | |
| | GGGCAGTGCCCCCACCACTGTGATCTATGAGGATAACCAGAGACCCTCTGGGGTCCCTGAT | | SGVPDRFSGSIDSSSNSASLTISGLKTE | | | |
| | CGTTTCTCTGGCTCCATCGACAGCTCCTCCAACTCTGCCTCCCTCACCATCTCTGGACTGA | | DEADYYCQSYDTRLRGVFGGGTKLTVL | | | |
| | AGACTGAGGACGAGGCTGACTACTACTGTCAGTCTTATGATACCAGGCTTCGAGGCGTTTT | | | | | |
| | CGGCGGAGGGACCAAGCTCACCGTCCTA | | | | | |
| ADI-75624 | GAAATTGTGATGACGCAGTCTCCACTCTCCCTGCCCGTCACCCCTGGAGAGCCGGCCTCCA | 3035 | EIVMTQSPLSLPVTPGEPASISCKSSQS | 3317 | | |
| | TCTCCTGCAAGTCTAGTCAGAGCCTCCTGCATAGTAATGGATACAAGTATTTGGATTGGTA | | LLHSNGYKYLDWYLQKPGQSPQLLIYLG | | | |
| | CCTGCAGAAGCCAGGGCAGTCTCCACAGCTCCTGATCTATTTGGGTTCTAATCGGGCCTCC | | SNRASGVPDRFSGSGSGTDFTLKISRVE | | | |
| | GGGGTCCCTGACAGGTTCAGTGGCAGTGGATCAGGCACAGATTTTACACTGAAAATCAGCA | | AEDVGIYYCMQALQTPITFGQGTRLEIK | | | |
| | GAGTGGAGGCTGAGGATGTTGGGATTTATTACTGCATGCAAGCTCTACAAACTCCGATCAC | | | | | |
| | CTTCGGCCAAGGGACACGACTGGAGATTAAA | | | | | |
| ADI-75764 | GACATCCAGTTGACCCAGTCTCCATCCTCCCTGTCTGCATCTGTTGGAGACAGAGTCACCA | 3036 | DIQLTQSPSSLSASVGDRVTITCQASQD | 3318 | | |
| | TCACTTGCCAGGCGAGTCAGGACATTGGCAATTATTTAAGTTGGTTTCAGCAGAAAGCAGG | | IGNYLSWFQQKAGKAPRLLMYEASNLET | | | |
| | GAAAGCCCCTAGGCTCCTGATGTACGAAGCATCCAATTTGGAAACAGGTGTCCCATCAAGG | | GVPSRFSGSGSGTDFALTISSLQPEDIA | | | |
| | TTCAGTGGAAGTGGATCTGGGACAGATTTTGCGCTCACCATCAGCAGCCTGCAGCCGGAAG | | TYYCQQYDSLLTFGPGTKVDIK | | | |
| | ATATTGCAACATATTACTGTCAACAGTATGACAGTCTACTCACTTTCGGCCCTGGGACCAA | | | | | |
| | GGTGGATATCAAA | | | | | |
| ADI-75806 | GACATCCAATTGACCCAGTCTCCATCCTCCCTGTCTGCATCTGTAGGAGACAGAGTCACCA | 3037 | DIQLTQSPSSLSASVGDRVTITCRASQS | 3319 | | |
| | TCACTTGCCGGGCAAGTCAGAGCGTTAACAACTATTTAAATTGGTTTCAGCAGAAACCAGG | | VNNYLNWFQQKPGKAPKLLIYAASSLQS | | | |
| | GAAAGCCCCTAAGCTCCTGATCTATGCTGCATCCAGCTTGCAAAGTGGGGTCCCCTCAAGG | | GVPSRFSGSGSGTDYTLAISSLQPEDFA | | | |
| | TTCAGTGGCAGTGGATCTGGGACAGATTACACTCTCGCCATCAGCAGTCTACAACCTGAAG | | TYYCQQSYATPLTFGPGTKVEIK | | | |
| | ATTTTGCAACTTACTACTGTCAACAGAGTTACGCTACCCCTCTCACTTTCGGCCCTGGGAC | | | | | |
| | CAAGGTGGAAATCAAA | | | | | |
| ADI-75630 | GACATCCCATTGACCCAGTCTCCATCCTCCCTGTCTGCATCTGTAGGAGACAGAGTCACCA | 3038 | DIPLTQSPSSLSASVGDRVTITCRASQD | 3320 | | |
| | TCACTTGCCGGGCGAGTCAGGACATTAGCAATTCTTTAGCCTGGTATCAGCAGAAACCAGG | | ISNSLAWYQQKPGKAPKLLLYTASTLES | | | |
| | GAAAGCCCCTAAGCTCCTGCTCTATACTGCTTCCACATTGGAAAGTGGGGTCCCATCCAGG | | GVPSRFSGSGSGTDYTLTISSLQPEDFA | | | |
| | TTCAGTGGCAGTGGATCTGGGACCGATTACACTCTCACCATCAGCAGCCTGCAGCCTGAAG | | TYYCQQYYSALGRTFGGGTKVDIK | | | |
| | ATTTTGCAACTTATTACTGTCAGCAGTATTATAGTGCCCTCGGGCGCACTTTCGGCGGAGG | | | | | |
| | GACCAAAGTGGATATCAAA | | | | | |
| ADI-75609 | CAGTCTGTGCTGACTCAGCCGCCCTCGGTGTCTGCAGCCCCCAGGCAGAGGGTCACCATCT | 3039 | QSVLTQPPSVSAAPRQRVTISCSGSSSN | 3321 | | |
| | CCTGTTCTGGCAGCAGCTCCAACATCGAGATTAATGCTGTAAACTGGTACCAGCAACTCCC | | IEINAVNWYQQLPGKAPKLLIYYDDLLP | | | |
| | AGGAAAGGCTCCCAAGCTCCTCATCTATTATGATGATCTGTTGCCCTCAGGGGTCTCTGAC | | SGVSDRFSGSKSGTSASLAISGLQSEDE | | | |
| | CGATTCTCTGGCTCCAAGTCTGGCACCTCAGCCTCCCTGGCCATCAGTGGGCTCCAGTCTG | | ADYYCVTFDDRLDGWVFGGGTKLTVL | | | |
| | AGGATGAGGCTGATTATTACTGTGTCACATTTGATGACAGGCTGGATGGCTGGGTGTTCGG | | | | | |
| | CGGAGGGACCAAGCTCACCGTCCTA | | | | | |
| ADI-75671 | GAAATCAAGATGACGCAGTCTCCATCCTCCCTGCCTGCATCTGTAGGAGACAGAGTCACCA | 3040 | EIKMTQSPSSLPASVGDRVTITCRASQS | 3322 | | |
| | TCACTTGCCGGGCAAGTCAGAGCATTACCAGTTATTTAAATTGGTATCAGCAGAAACCAGG | | ITSYLNWYQQKPGKAPKLLIYGASSLHT | | | |
| | GAAAGCCCCTAAGCTCCTGATCTATGGTGCATCCAGTTTGCATACTGGGGTCCCCTCAAGG | | GVPSRFSGSGSGTHFTLTISSLQPEDFA | | | |
| | TTCAGTGGCAGTGGATCTGGGACACATTTCACTCTCACCATCAGCAGTCTGCAACCTGAAG | | TYFCQQSYTTPRTFGQGTKVEIK | | | |
| | ATTTTGCAACTTACTTCTGTCAACAGAGTTACACTACCCCTCGGACTTTTGGCCAGGGGAC | | | | | |
| | CAAGGTGGAAATCAAA | | | | | |
| ADI-75660 | GAAATCCAGATGACCCAGTCTCCATCCTCCCTGTCTGCATCTGTTGGAGACAGAGTCACCA | 3041 | EIQMTQSPSSLSASVGDRVTITCRASQG | 3323 | | |
| | TCACTTGCCGGGCAAGTCAGGGCATTAGCTTTGCTTTAGCCTGGTATCAGCAAAAACCAGG | | ISFALAWYQQKPGKAPKLLIYDASSLES | | | |
| | GAAAGCTCCTAAGCTCCTGATCTATGATGCCTCCAGTTTGGAAAGTGGGGTCCCATCAAGG | | GVPSRFSGSGSGTDFTLTISSLQPEDFA | | | |
| | TTCAGCGGCAGTGGATCTGGGACAGATTTCACTCTCACCATCAGCAGCCTGCAGCCTGAAG | | TYYCQQFNHYPITFGQGTRLEIK | | | |
| | ATTTTGCAACTTATTACTGTCAACAGTTTAATCATTATCCGATCACCTTCGGCCAAGGGAC | | | | | |
| | ACGACTGGAGATTAAA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75802 | IGKV1D- | EIVMTQ | 1891 | RASQSIR | 2117 | WFQQKP | 2300 | SASTLQ | 2435 | GVPSRFSG | 2580 | QQSYN | 2702 | FGQGTK | 2937 |
| | 39 | SPSSLS | | SYLN | | GKAPKL | | S | | SASGTDFT | | TPPWT | | VEIK | |
| | | ASVGDR | | | | LIY | | | | LTISSLQP | | | | | |
| | | VTITC | | | | | | | | EDFATYYC | | | | | |
| ADI-75614 | IGKV4-1 | DIQMTQ | 1927 | MSSQSVL | 2118 | WYQQRP | 2302 | WASTRK | 2436 | GVPDRFSG | 2581 | QQYYR | 2771 | FGGGTK | 2936 |
| | | SPDSLA | | DSSNNKN | | GQPPKL | | S | | SGSGTDFT | | TPLT | | VEIK | |
| | | VSLGER | | FLA | | IIY | | | | LTISSLQP | | | | | |
| | | ATINC | | | | | | | | EDVAVYYC | | | | | |
| ADI-75661 | IGKV1-5 | DIQMTQ | 1928 | RASQSIS | 2052 | WYQQKP | 2257 | MASSLE | 2437 | GVPSRFSG | 2532 | QQYNS | 2772 | FGQGTK | 2937 |
| | | SPSTLS | | RWLA | | GKAPKL | | S | | SGSGTEFT | | YWT | | VEIK | |
| | | ASVGDR | | | | LIY | | | | LTISSLQP | | | | | |
| | | VTISC | | | | | | | | DDFATYYC | | | | | |
| ADI-75697 | IGKV1- | DIQMTQ | 1929 | RASQGIS | 2062 | WYQQKP | 2303 | VASTWE | 2438 | GVPTRFSG | 2582 | QQYYS | 2773 | FGGGTK | 2936 |
| | NL1 | SPSSLS | | NSLA | | GKAPKL | | S | | SGYGTDFT | | PPPPT | | VEIK | |
| | | ASVGDG | | | | LVY | | | | LTISSLQP | | | | | |
| | | VTITC | | | | | | | | EDFATYYC | | | | | |
| ADI-75700 | IGKV3-11 | EIALTQ | 1930 | RASQSVS | 2119 | WYQQKP | 2258 | DASKRA | 2439 | GIPARFNG | 2583 | QQRST | 2774 | FGGGTK | 2938 |
| | | SPATLS | | TYLA | | GQAPRL | | T | | SGSGTDFT | | WPT | | VDIK | |
| | | LSPGER | | | | LIY | | | | LTISSLEP | | | | | |
| | | ATLSC | | | | | | | | EDFAVYYC | | | | | |
| ADI-75787 | IGKV1D- | DIQLTQ | 1876 | RASQSIG | 2120 | WYQQKP | 2263 | AASNLQ | 2408 | GVPSRFSG | 2567 | QQSYS | 2775 | FGQGTK | 2937 |
| | 39 | SPSSLS | | SYLN | | GKAPNL | | S | | SGSGTDFT | | APPWT | | VEIK | |
| | | ASVGDR | | | | LIY | | | | LTISSLQP | | | | | |
| | | VTITC | | | | | | | | EDFATYFC | | | | | |
| ADI-75642 | IGLV3-21 | QSVLTQ | 1931 | GGNNIGS | 2121 | WYQQKP | 2304 | YDNDRP | 2440 | GIPERFSG | 2584 | QVWDT | 2776 | FGGGTK | 2939 |
| | | PPSVSV | | QSVH | | GQAPVL | | S | | SNSGNTAT | | SGDHW | | LTVL | |
| | | APGKTA | | | | VIY | | | | LTISRVEA | | V | | | |
| | | RITQ | | | | | | | | GDEADFYC | | | | | |
| ADI-75861 | IGKV1D- | DIQMTQ | 1907 | RASQSVS | 2122 | WYQQKP | 2257 | SASGLQ | 2441 | GVPSRFSG | 2534 | QQSYN | 2777 | FGPGTK | 2942 |
| | 39 | SPSSLS | | TFLN | | GKAPKL | | S | | SGSGTDFT | | MFT | | VEIK | |
| | | ASIGDR | | | | LIY | | | | LTISSLQP | | | | | |
| | | VTITC | | | | | | | | EDFATYYC | | | | | |
| ADI-75578 | IGKV1D- | DIPMTQ | 1932 | RASQTIT | 2123 | WYQQKP | 2257 | AASSLQ | 2391 | GVPSRFSG | 2567 | QQSYT | 2749 | FGQGTK | 2944 |
| | 39 | SPSSLS | | RYLN | | GKAPKL | | S | | SGSGTDFT | | THMYT | | VDIK | |
| | | ASVGDR | | | | LIY | | | | LTISSLQP | | | | | |
| | | VTISC | | | | | | | | EDFATYFC | | | | | |
| ADI-75808 | IGLV2-14 | QPVLTQ | 1933 | TGTSSDL | 2124 | WYQHFP | 2305 | EFTNRP | 2442 | GVSNRFSG | 2585 | SAYTS | 2778 | FGGGTK | 2947 |
| | | PASVSG | | GAFNHVS | | GKAPKL | | S | | SKSDKTAS | | TSTV | | VTVL | |
| | | SPGQSI | | | | LIY | | | | LTIFGLQA | | | | | |
| | | TISC | | | | | | | | EDEADYYC | | | | | |
| ADI-75848 | IGKV1- | EIVMTQ | 1934 | RASQGIS | 2062 | WYQQKP | 2259 | AASTLE | 2398 | GVPSRFSG | 2536 | QQYYS | 2779 | FGQGTK | 2937 |
| | NL1 | SPSSLS | | NSLA | | GKAPKL | | S | | SGSGTDFT | | PPPRT | | VEIK | |
| | | ASVGDR | | | | LLY | | | | LTINSLQP | | | | | |
| | | ITITC | | | | | | | | EDFATYYC | | | | | |
| ADI-75832 | IGKV3-20 | EIVMTQ | 1935 | RASQSV- | 2125 | WYQQRP | 2306 | DASSRA | 2443 | GIPDRFSG | 2586 | QQYGS | 2780 | FGQGTK | 2937 |
| | | SPGTLS | | SGYLA | | GQAPRL | | T | | SGSGTDFT | | SPRT | | VEIK | |
| | | LSPGER | | | | LIY | | | | LTISRLEP | | | | | |
| | | ATLSC | | | | | | | | EDFAVYFC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75802 | GAAATTGTGATGACGCAGTCTCCATCCTCCCTGTCTGCATCTGTAGGAGACAGAGTCACCA | 3042 | EIVMTQSPSSLSASVGDRVTITCRASQS | 3324 | | |
| | TCACTTGCCGGGCAAGTCAGAGCATTAGAAGTTATTTAAATTGGTTTCAACAGAAACCAGG | | IRSYLNWFQQKPGKAPKLLIYSASTLQS | | | |
| | AAAAGCCCCTAAACTCCTTATCTATAGTGCATCCACTTTGCAAAGTGGGGTCCCATCAAGG | | GVPSRFSGSASGTDFTLTISSLQPEDFA | | | |
| | TTCAGTGGCAGTGCATCTGGGACAGATTTCACTCTCACCATCAGCAGTCTGCAACCTGAAG | | TYYCQQSYNTPPWTFGQGTKVEIK | | | |
| | ATTTTGCAACTTACTACTGTCAACAGAGTTACAATACCCCTCCGTGGACGTTCGGCCAAGG | | | | | |
| | GACCAAGGTGGAGATCAAA | | | | | |
| ADI-75614 | GACATCCAAATGACCCAGTCTCCAGACTCCCTGGCTGTGTCTCTGGGCGAGAGGGCCACCA | 3043 | DIQMTQSPDSLAVSLGERATINCMSSQS | 3325 | | |
| | TCAACTGCATGTCCAGCCAGAGTGTTTTAGACAGCTCCAACAATAAGAACTTCTTAGCTTG | | VLDSSNNKNFLAWYQQRPGQPPKLIIYW | | | |
| | GTACCAGCAGAGACCAGGACAGCCTCCTAAGCTGATCATTTACTGGGCATCTACCCGGAAA | | ASTRKSGVPDRFSGSGSGTDFTLTISSL | | | |
| | TCCGGGGTCCCTGACCGATTCAGTGGCAGCGGGTCTGGGACAGATTTCACTCTCACAATCA | | QPEDVAVYYCQQYYRTPLTFGGGTKVEI | | | |
| | GCAGCCTGCAGCCTGAAGATGTGGCAGTTTATTACTGTCAGCAATATTACAGAACTCCTCT | | K | | | |
| | CACTTTCGGCGGAGGGACCAAGGTGGAAATCAAA | | | | | |
| ADI-75661 | GACATCCAGATGACCCAGTCTCCTTCCACCCTGTCTGCATCTGTAGGGGACAGAGTCACCA | 3044 | DIQMTQSPSTLSASVGDRVTISCRASQS | 3326 | | |
| | TCAGTTGCCGGGCCAGTCAGAGTATTAGTAGGTGGTTGGCCTGGTATCAGCAGAAACCAGG | | ISRWLAWYQQKPGKAPKLLIYMASSLES | | | |
| | GAAAGCCCCTAAGCTCCTGATCTATATGGCGTCCAGTTTAGAAAGTGGGGTCCCATCGAGG | | GVPSRFSGSGSGTEFTLTISSLQPDDFA | | | |
| | TTCAGCGGCAGTGGATCTGGGACAGAATTCACTCTCACCATCAGCAGCCTTCAGCCTGATG | | TYYCQQYNSYWTFGQGTKVEIK | | | |
| | ATTTTGCAACTTATTACTGCCAACAATATAATAGTTATTGGACGTTCGGCCAAGGGACCAA | | | | | |
| | GGTGGAAATCAAA | | | | | |
| ADI-75697 | GACATCCAGATGACCCAGTCTCCATCCTCCCTGTCTGCATCTGTTGGAGACGGAGTCACCA | 3045 | DIQMTQSPSSLSASVGDGVTITCRASQG | 3327 | | |
| | TCACTTGCCGGGCGAGTCAGGGGATTAGCAATTCTTTAGCTTGGTATCAGCAGAAACCGGG | | ISNSLAWYQQKPGKAPKLLVYVASTWES | | | |
| | GAAAGCCCCTAAGCTCCTCGTCTATGTCGCATCCACATGGGAAAGTGGGGTCCCAACCAGG | | GVPTRFSGSGYGTDFTLTISSLQPEDFA | | | |
| | TTCAGTGGCAGTGGATATGGGACGGATTTCACTCTCACCATCAGCAGCCTGCAGCCTGAAG | | TYYCQQYYSPPPPTFGGGTKVEIK | | | |
| | ATTTTGCAACTTATTACTGTCAACAGTATTATAGTCCCCCGCCGCCCACCTTCGGCGGAGG | | | | | |
| | GACCAAGGTGGAGATCAAA | | | | | |
| ADI-75700 | GAAATCGCGCTGACGCAGTCTCCAGCCACCCTGTCTTTGTCTCCAGGGGAAAGAGCCACCC | 3046 | EIALTQSPATLSLSPGERATLSCRASQS | 3328 | | |
| | TCTCCTGCAGGGCCAGTCAGAGTGTTAGCACCTACTTAGCCTGGTACCAACAGAAACCTGG | | VSTYLAWYQQKPGQAPRLLIYDASKRAT | | | |
| | CCAGGCTCCCAGGCTCCTCATCTATGATGCATCCAAGAGGGCCACTGGCATCCCAGCCAGG | | GIPARFNGSGSGTDFTLTISSLEPEDFA | | | |
| | TTCAATGGCAGTGGGTCTGGGACAGACTTCACTCTCACCATCAGCAGCCTCGAGCCTGAAG | | VYYCQQRSTWPTFGGGTKVDIK | | | |
| | ATTTTGCAGTTTATTACTGTCAGCAGCGTAGCACCTGGCCCACTTTCGGCGGAGGGACCAA | | | | | |
| | GGTGGATATCAAA | | | | | |
| ADI-75787 | GACATCCAGTTGACCCAGTCTCCATCCTCCCTGTCTGCATCTGTAGGAGACAGAGTCACCA | 3047 | DIQLTQSPSSLSASVGDRVTITCRASQS | 3329 | | |
| | TCACTTGCCGGGCAAGTCAGAGCATTGGCAGCTATTTAAATTGGTATCAGCAGAAACCAGG | | IGSYLNWYQQKPGKAPNLLIYAASNLQS | | | |
| | GAAAGCCCCTAACCTCCTGATCTATGCTGCATCCAATTTGCAAAGTGGAGTCCCATCAAGG | | GVPSRFSGSGSGTDFTLTISSLQPEDFA | | | |
| | TTCAGTGGCAGTGGATCTGGGACAGATTTCACTCTCACCATCAGCAGTCTGCAGCCTGAAG | | TYFCQQSYSAPPWTFGQGTKVEIK | | | |
| | ATTTTGCAACTTACTTCTGTCAGCAGAGTTACAGTGCCCCTCCTTGGACATTCGGCCAGGG | | | | | |
| | GACCAAGGTGGAGATCAAA | | | | | |
| ADI-75642 | CAGTCTGTGCTGACTCAGCCACCCTCAGTGTCAGTGGCCCCAGGAAAGACGGCCAGGATTA | 3048 | QSVLTQPPSVSVAPGKTARITCGGNNIG | 3330 | | |
| | CCTGTGGGGGAAACAACATTGGAAGTCAAAGTGTGCACTGGTACCAGCAGAAGCCAGGCCA | | SQSVHWYQQKPGQAPVLVIYYDNDRPSG | | | |
| | GGCCCCCGTGCTGGTCATCTATTATGATAACGACCGGCCCTCTGGGATCCCTGAGCGATTC | | IPERFSGSNSGNTATLTISRVEAGDEAD | | | |
| | TCTGGCTCCAACTCTGGGAACACGGCCACCCTGACCATCAGCAGGGTCGAAGCCGGGGATG | | FYCQVWDTSGDHWVFGGGTKLTVL | | | |
| | AGGCCGACTTTTACTGTCAGGTGTGGGATACTAGTGGTGATCATTGGGTGTTCGGCGGAGG | | | | | |
| | CACCAAGCTCACCGTCCTA | | | | | |
| ADI-75861 | GACATCCAGATGACCCAGTCTCCATCCTCCCTGTCTGCATCTATAGGAGACAGAGTCACCA | 3049 | DIQMTQSPSSLSASIGDRVTITCRASQS | 3331 | | |
| | TCACTTGCCGGGCAAGTCAGAGCGTTAGCACCTTTTTAAATTGGTATCAGCAGAAGCCAGG | | VSTFLNWYQQKPGKAPKLLIYSASGLQS | | | |
| | AAAAGCCCCAAAGCTCCTGATTTATTCTGCATCCGGTTTGCAAAGTGGGGTCCCATCAAGG | | GVPSRFSGSGSGTDFTLTISSLQPEDFA | | | |
| | TTCAGTGGCAGTGGATCTGGGACAGATTTCACTCTCACCATCAGCAGTCTGCAACCTGAAG | | TYYCQQSYNMFTFGPGTKVEIK | | | |
| | ATTTTGCAACTTACTACTGTCAACAGAGTTACAATATGTTCACTTTCGGCCCTGGGACCAA | | | | | |
| | GGTGGAAATCAAA | | | | | |
| ADI-75578 | GACATCCCGATGACCCAGTCTCCATCCTCCCTGTCTGCATCTGTAGGAGACAGAGTCACCA | 3050 | DIPMTQSPSSLSASVGDRVTISCRASQT | 3332 | | |
| | TCTCTTGCCGGGCAAGTCAGACCATTACCAGGTATTTAAATTGGTATCAGCAAAAACCAGG | | ITRYLNWYQQKPGKAPKLLIYAASSLQS | | | |
| | GAAAGCCCCTAAGCTCCTGATCTATGCTGCATCCAGTTTGCAAAGTGGGGTCCCATCAAGG | | GVPSRFSGSGSGTDFTLTISSLQPEDFA | | | |
| | TTCAGTGGCAGTGGATCTGGGACAGATTTCACTCTCACCATCAGCAGTCTGCAACCTGAGG | | TYFCQQSYTTHMYTFGQGTKVDIK | | | |
| | ATTTTGCAACTTACTTCTGTCAGCAGAGTTATACTACCCACATGTACACTTTTGGCCAGGG | | | | | |
| | GACCAAGGTGGATATCAAA | | | | | |
| ADI-75808 | CAGCCTGTGCTGACTCAGCCTGCCTCCGTGTCTGGGTCTCCTGGACAGTCGATCACCATCT | 3051 | QPVLTQPASVSGSPGQSITISCTGTSSD | 3333 | | |
| | CCTGCACTGGAACCAGCAGTGACCTTGGTGCTTTTAACCATGTCTCCTGGTACCAACACTT | | LGAFNHVSWYQHFPGKAPKLLIYEFTNR | | | |
| | CCCAGGCAAGGCCCCCAAACTTTTGATTTATGAGTTCACTAATCGGCCCTCAGGGGTTTCT | | PSGVSNRFSGSKSDKTASLTIFGLQAED | | | |
| | AATCGCTTCTCCGGCTCCAAGTCTGACAAGACGGCCTCCCTGACCATCTTTGGGCTCCAGG | | EADYYCSAYTSTSTVFGGGTKVTVL | | | |
| | CTGAGGACGAGGCTGATTATTACTGCAGTGCATATACATCCACCAGCACAGTATTCGGCGG | | | | | |
| | AGGGACCAAGGTCACCGTCCTA | | | | | |
| ADI-75848 | GAAATTGTGATGACCCAGTCTCCATCCTCCCTGTCTGCATCTGTAGGAGACAGAATCACCA | 3052 | EIVMTQSPSSLSASVGDRITITCRASQG | 3334 | | |
| | TCACTTGCCGGGCGAGTCAGGGCATTAGCAATTCTTTAGCCTGGTATCAGCAGAAACCAGG | | ISNSLAWYQQKPGKAPKLLLYAASTLES | | | |
| | GAAAGCCCCTAAGCTCCTGCTCTATGCTGCATCCACATTGGAAAGTGGGGTCCCATCCAGG | | GVPSRFSGSGSGTDFTLTINSLQPEDFA | | | |
| | TTCAGTGGCAGTGGATCTGGGACGGATTTCACTCTCACCATCAACAGCCTGCAGCCTGAAG | | TYYCQQYYSPPPRTFGQGTKVEIK | | | |
| | ATTTTGCAACTTATTACTGTCAACAGTATTATAGTCCCCCTCCCCGGACGTTCGGCCAAGG | | | | | |
| | GACCAAGGTGGAAATCAAA | | | | | |
| ADI-75832 | GAAATTGTGATGACCCAGTCTCCAGGCACCCTGTCTTTGTCTCCAGGGGAAAGAGCCACCC | 3053 | EIVMTQSPGTLSLSPGERATLSCRASQS | 3335 | | |
| | TCTCCTGCAGGGCCAGTCAGAGTGTTAGCGGCTACTTAGCCTGGTACCAGCAGAGACCTGG | | VSGYLAWYQQRPGQAPRLLIYDASSRAT | | | |
| | CCAGGCTCCCAGGCTCCTCATCTATGATGCATCCAGCAGGGCCACTGGCATCCCAGACAGG | | GIPDRFSGSGSGTDFTLTISRLEPEDFA | | | |
| | TTCAGTGGCAGTGGGTCTGGGACAGACTTCACTCTCACCATCAGCAGACTGGAGCCTGAGG | | VYFCQQYGSSPRTFGQGTKVEIK | | | |
| | ATTTTGCAGTGTATTTCTGTCAGCAGTATGGTAGCTCACCTCGAACGTTCGGCCAAGGGAC | | | | | |
| | CAAGGTGGAAATCAAA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75618 | IGKV1-5 | DIQMTQ | 1879 | RASQSID | 2126 | WYQQKP | 2257 | KTSSLE | 2444 | GVPSRFSG | 2532 | QHFNS | 2781 | FGQGTK | 2944 |
| | | SPSTLS | | DWLA | | GKAPKL | | S | | SGSGTEFT | | YLMYT | | VDIK | |
| | | ASVGDR | | | | LIY | | | | LTISSLQP | | | | | |
| | | VTITC | | | | | | | | DDFATYYC | | | | | |
| ADI-75830 | IGLV3-21 | QPVLTQ | 1936 | GAHDIGS | 2127 | WYQQKP | 2283 | DDSDRP | 2399 | GIPERVSG | 2587 | QVWDT | 2776 | FGGGTK | 2939 |
| | | PPSVSV | | KSVH | | GQAPVL | | S | | SNSGNTAT | | SGDHW | | LTVL | |
| | | APGQTA | | | | VVY | | | | LTISRVEA | | V | | | |
| | | GITC | | | | | | | | GDEADYYC | | | | | |
| ADI-75628 | IGKV1-33 | DIRMTQ | 1937 | QASQDIN | 2128 | WYQQKP | 2257 | DASNLE | 2416 | GVPSRFSG | 2588 | QSYDN | 2782 | FGPGTK | 2942 |
| | | SPSSLS | | TYLN | | GKAPKL | | T | | SGSGTHFT | | PPPT | | VEIK | |
| | | ASVGDR | | | | LIY | | | | FTITSLQP | | | | | |
| | | VTITC | | | | | | | | EDIATYYC | | | | | |
| ADI-75640 | IGKV3D- | DMTMTQ | 1938 | RASQSIS | 2129 | WYQQKP | 2258 | GASTRA | 2383 | GISARFSG | 2589 | QQYNN | 2783 | FGQGTK | 2937 |
| | 15 | SPATLS | | SNLA | | GQAPRL | | T | | SGSGTKFT | | WPPWT | | VEIK | |
| | | VSLGER | | | | LIY | | | | LTISSLQS | | | | | |
| | | VTLSC | | | | | | | | EDFAVYYC | | | | | |
| ADI-75657 | IGKV2D- | DIVMTQ | 1939 | KSSQSLL | 2130 | WYLQKP | 2260 | LGSNRA | 2387 | GVPDRFSG | 2575 | MQALQ | 2765 | FGQGTR | 2940 |
| | 28 | SPLTLP | | HSNGHKY | | GQSPQL | | S | | SGSGTDFT | | TPIT | | LEIK | |
| | | VTPGEP | | LD | | LIY | | | | LKISRVEA | | | | | |
| | | ASISC | | | | | | | | EDVGIYYC | | | | | |
| ADI-75713 | IGLV1-51 | QSVVTQ | 1940 | SGTSSNI | 2131 | WYQHLP | 2307 | DNDKRP | 2445 | GIPDRFSG | 2590 | ATWDD | 2784 | FGGGTQ | 2948 |
| | | PPSVSA | | GNNYVS | | GTAPKL | | S | | SKSGTSAT | | SLSVG | | LTVL | |
| | | APGQRV | | | | LIY | | | | LGITGLQS | | I | | | |
| | | TISC | | | | | | | | GDEADYYC | | | | | |
| ADI-75692 | IGLV1-36 | QPVLTQ | 1941 | FGSSSNI | 2132 | WYQQLP | 2308 | YDDLLP | 2433 | GVSDRFSG | 2578 | AAWDD | 2785 | FGPGTK | 2951 |
| | | PPSVSE | | GHNAVN | | GKAPKL | | S | | SKSGTSAS | | RLNGY | | VTVL | |
| | | APRQRV | | | | IIY | | | | LAISGLQS | | V | | | |
| | | TISC | | | | | | | | EDEADYYC | | | | | |
| ADI-75587 | IGKV1D- | ASVGDR | 1912 | RASQTIS | 2133 | WYQQKP | 2257 | AASSLQ | 2391 | GVPSRFSG | 2534 | QQSYS | 2775 | FAQGTK | 2952 |
| | 39 | EIPMTQ | | RFLN | | GKAPKL | | S | | SGSGTDFT | | APPWT | | VDIK | |
| | | SPSSLS | | | | LIY | | | | LTISSLQP | | | | | |
| | | VTITC | | | | | | | | EDFATYYC | | | | | |
| ADI-75775 | IGKV1D- | DIQMTQ | 1895 | RASQSIS | 2134 | WYQQRP | 2309 | AASTLQ | 2392 | GVPSRFSG | 2591 | QQSYV | 2786 | FGPGTK | 2942 |
| | 39 | SPSSLS | | RYLN | | GKAPKL | | S | | SGSGTDFT | | SPPWT | | VEIK | |
| | | ASVGDR | | | | LIY | | | | LTISSLQP | | | | | |
| | | VTISC | | | | | | | | EDFATYIC | | | | | |
| ADI-75758 | IGKV1D- | DIQMTQ | 1878 | RASQRIS | 2135 | WYQQRP | 2310 | NASSFE | 2446 | GVPSRFSG | 2534 | QQSYS | 2787 | FGQGTK | 2937 |
| | 39 | SPSSLS | | SYLN | | GKAPNF | | S | | SGSGTDFT | | DPYT | | VEIK | |
| | | ASVGDR | | | | LIY | | | | LTISSLQP | | | | | |
| | | VTITC | | | | | | | | EDFATYYC | | | | | |
| ADI-75793 | IGKV1-9 | DIKMTQ | 1942 | RASQGIS | 2136 | WYQQKP | 2257 | AASTLQ | 2392 | GVPSRFSG | 2592 | QQLNT | 2788 | FGPGTK | 2942 |
| | | SPSFLS | | SYLA | | GKAPKL | | S | | SGSGTEFT | | HPPP | | VEIK | |
| | | ASVGDR | | | | LIY | | | | LTISSLQP | | | | | |
| | | VTITC | | | | | | | | EDFATYYC | | | | | |
| ADI-75804 | IGKV1D- | DIVMTQ | 1902 | RASQSIS | 2066 | WYQQKP | 2311 | AASSLQ | 2391 | GVPSRFSG | 2534 | QQSYT | 2789 | FGQGTK | 2937 |
| | 39 | SPSSLS | | TYLN | | EKAPKL | | S | | SGSGTDFT | | SPAYT | | VEIK | |
| | | ASVGDR | | | | LIY | | | | LTISSLQP | | | | | |
| | | VTITC | | | | | | | | EDFATYYC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75618 | GACATCCAGATGACCCAGTCTCCTTCCACCCTGTCTGCATCTGTGGGAGACAGAGTCACCA | 3054 | DIQMTQSPSTLSASVGDRVTITCRASQS | 3336 | | |
| | TCACTTGCCGGGCCAGTCAGAGTATTGATGACTGGTTGGCCTGGTATCAGCAGAAACCAGG | | IDDWLAWYQQKPGKAPKLLIYKTSSLES | | | |
| | GAAAGCCCCTAAACTCCTGATCTATAAGACGTCTAGTTTAGAAAGTGGGGTCCCATCAAGA | | GVPSRFSGSGSGTEFTLTISSLQPDDFA | | | |
| | TTCAGCGGCAGTGGATCTGGGACAGAATTCACTCTCACCATCAGCAGCCTGCAGCCTGATG | | TYYCQHFNSYLMYTFGQGTKVDIK | | | |
| | ATTTTGCAACTTATTACTGCCAACACTTTAATAGTTACCTCATGTACACTTTTGGCCAGGG | | | | | |
| | GACCAAGGTGGATATCAAA | | | | | |
| ADI-75830 | CAGCCTGTGCTGACTCAGCCCCCCTCGGTGTCAGTGGCCCCAGGACAGACGGCCGGGATTA | 3055 | QPVLTQPPSVSVAPGQTAGITCGAHDIG | 3337 | | |
| | CCTGTGGGGCACACGACATTGGAAGTAAGAGTGTGCACTGGTACCAGCAAAAGCCAGGCCA | | SKSVHWYQQKPGQAPVLVVYDDSDRPSG | | | |
| | GGCCCCTGTGCTGGTCGTCTATGATGATAGCGACCGGCCCTCAGGGATCCCTGAGCGAGTC | | IPERVSGSNSGNTATLTISRVEAGDEAD | | | |
| | TCTGGCTCCAACTCTGGAAACACGGCCACCCTGACCATCAGCAGGGTCGAAGCCGGGGATG | | YYCQVWDTSGDHWVFGGGTKLTVL | | | |
| | AGGCCGACTATTACTGTCAGGTGTGGGATACTAGTGGTGATCATTGGGTGTTCGGCGGAGG | | | | | |
| | GACCAAGCTCACCGTCCTA | | | | | |
| ADI-75628 | GACATCCGGATGACCCAGTCTCCATCCTCCCTGTCTGCATCTGTAGGAGACAGAGTCACCA | 3056 | DIRMTQSPSSLSASVGDRVTITCQASQD | 3338 | | |
| | TCACTTGCCAGGCGAGTCAGGACATTAACACCTATTTAAATTGGTATCAGCAGAAACCAGG | | INTYLNWYQQKPGKAPKLLIYDASNLET | | | |
| | GAAAGCCCCTAAACTCCTGATCTACGATGCATCCAATTTGGAAACAGGGGTCCCATCAAGG | | GVPSRFSGSGSGTHFTFTITSLQPEDIA | | | |
| | TTCAGTGGAAGTGGATCTGGGACACATTTTACTTTCACCATCACCAGCCTGCAGCCTGAAG | | TYYCQSYDNPPPTFGPGTKVEIK | | | |
| | ACATTGCAACATATTACTGTCAATCCTATGATAATCCCCCTCCGACTTTCGGCCCTGGGAC | | | | | |
| | CAAGGTGGAGATCAAA | | | | | |
| ADI-75640 | GACATGACGATGACGCAGTCTCCAGCCACCCTGTCTGTGTCTCTAGGAGAAAGAGTCACCC | 3057 | DMTMTQSPATLSVSLGERVTLSCRASQS | 3339 | | |
| | TCTCCTGCAGGGCCAGTCAGAGTATTAGCAGCAACTTAGCCTGGTACCAGCAGAAACCTGG | | ISSNLAWYQQKPGQAPRLLIYGASTRAT | | | |
| | CCAGGCTCCCAGGCTCCTCATCTATGGTGCATCCACCAGGGCCACTGGTATCTCAGCCAGG | | GISARFSGSGSGTKFTLTISSLQSEDFA | | | |
| | TTCAGTGGCAGTGGGTCTGGGACAAAATTCACTCTCACCATCAGCAGCCTGCAGTCTGAAG | | VYYCQQYNNWPPWTFGQGTKVEIK | | | |
| | ATTTTGCAGTTTATTACTGTCAGCAATATAATAACTGGCCTCCGTGGACGTTCGGCCAGGG | | | | | |
| | GACCAAGGTGGAAATCAAA | | | | | |
| ADI-75657 | GACATTGTGATGACGCAGTCTCCACTCACCCTGCCCGTCACCCCTGGAGAGCCGGCCTCCA | 3058 | DIVMTQSPLTLPVTPGEPASISCKSSQS | 3340 | | |
| | TCTCCTGCAAGTCTAGTCAGAGCCTCCTGCATAGTAATGGACACAAGTACTTGGATTGGTA | | LLHSNGHKYLDWYLQKPGQSPQLLIYLG | | | |
| | CCTGCAGAAGCCAGGGCAGTCTCCACAGCTCCTGATCTATTTGGGTTCTAATCGGGCCTCC | | SNRASGVPDRFSGSGSGTDFTLKISRVE | | | |
| | GGGGTCCCTGACAGGTTCAGTGGCAGTGGATCAGGCACAGATTTCACACTGAAAATCAGCA | | AEDVGIYYCMQALQTPITFGQGTRLEIK | | | |
| | GAGTGGAGGCTGAGGATGTTGGGATTTATTACTGCATGCAAGCTCTACAAACTCCGATCAC | | | | | |
| | CTTCGGCCAAGGGACACGACTGGAGATTAAA | | | | | |
| ADI-75713 | CAGTCAGTGGTGACTCAGCCACCCTCAGTGTCTGCGGCCCCAGGACAGAGGGTCACCATCT | 3059 | QSVVTQPPSVSAAPGQRVTISCSGTSSN | 3341 | | |
| | CCTGCTCTGGAACCAGCTCCAACATTGGGAATAATTATGTATCCTGGTACCAGCACCTCCC | | IGNNYVSWYQHLPGTAPKLLIYDNDKRP | | | |
| | AGGAACAGCCCCCAAACTCCTCATTTATGACAATGATAAGCGACCCTCAGGGATTCCTGAC | | SGIPDRFSGSKSGTSATLGITGLQSGDE | | | |
| | CGATTCTCTGGCTCCAAGTCTGGCACGTCAGCCACCCTGGGCATCACCGGTCTCCAGAGTG | | ADYYCATWDDSLSVGIFGGGTQLTVL | | | |
| | GGGACGAGGCCGATTATTATTGCGCTACATGGGATGACAGCCTGAGTGTGGGGATTTTCGG | | | | | |
| | CGGAGGCACCCAGCTGACCGTCCTC | | | | | |
| ADI-75692 | CAGCCTGTGCTGACTCAGCCCCCCTCGGTGTCTGAAGCAGGCAGAGGGTCACCATCT | 3060 | QPVLTQPPSVSEAPRQRVTISCFGSSSN | 3342 | | |
| | CCTGTTTTGGGAGCAGCTCCAACATCGGACATAATGCTGTAAACTGGTATCAGCAGCTCCC | | IGHNAVNWYQQLPGKAPKLIIYYDDLLP | | | |
| | AGGAAAGGCTCCCAAACTTATCATCTACTATGATGATCTGCTGCCCTCAGGGGTCTCCGAC | | SGVSDRFSGSKSGTSASLAISGLQSEDE | | | |
| | CGATTCTCTGGCTCCAAGTCTGGCACCTCAGCCTCCCTGGCCATCAGTGGGCTCCAGTCTG | | ADYYCAAWDDRLNGYVFGPGTKVTVL | | | |
| | AGGATGAGGCTGATTATTACTGTGCAGCATGGGATGACAGGCTGAATGGTTATGTCTTCGG | | | | | |
| | ACCTGGGACCAAGGTCACCGTCCTA | | | | | |
| ADI-75587 | GAAATCCCGATGACCCAGTCTCCATCCTCCCTGTCTGCATCTGTAGGAGACAGAGTCACCA | 3061 | EIPMTQSPSSLSASVGDRVTITCRASQT | 3343 | | |
| | TCACTTGCCGGGCAAGTCAGACCATTAGTAGGTTTTTAAATTGGTATCAGCAGAAACCAGG | | ISRFLNWYQQKPGKAPKLLIYAASSLQS | | | |
| | GAAAGCCCCTAAACTCCTGATCTATGCTGCATCCAGTTTGCAAAGTGGGGTCCCATCAAGG | | GVPSRFSGSGSGTDFTLTISSLQPEDFA | | | |
| | TTCAGTGGCAGTGGATCTGGGACAGATTTCACTCTCACCATCAGCAGTCTGCAACCTGAAG | | TYYCQQSYSAPPWTFAQGTKVDIK | | | |
| | ATTTTGCAACTTACTACTGTCAACAGAGTTACAGTGCCCCTCCGTGGACTTTTGCCCAGGG | | | | | |
| | GACCAAGGTGGATATCAAA | | | | | |
| ADI-75775 | GACATCCAGATGACCCAGTCTCCATCCTCCCTGTCTGCATCTGTAGGGGACAGAGTCACCA | 3062 | DIQMTQSPSSLSASVGDRVTISCRASQS | 3344 | | |
| | TCAGTTGCCGGGCAAGTCAGAGCATTAGCAGGTATTTAAATTGGTATCAGCAGAGACCAGG | | ISRYLNWYQQRPGKAPKLLIYAASTLQS | | | |
| | GAAAGCCCCTAAACTCCTGATCTATGCTGCATCCACTTTGCAAAGTGGGGTCCCATCAAGA | | GVPSRFSGSGSGTDFTLTISSLQPEDFA | | | |
| | TTCAGTGGCAGTGGATCTGGGACAGACTTCACTCTTACCATCAGCAGTCTGCAACCTGAAG | | TYICQQSYVSPPWTFGPGTKVEIK | | | |
| | ATTTTGCAACTTACATCTGTCAACAGAGTTACGTTAGCCCCCCGTGGACGTTCGGCCCAGG | | | | | |
| | GACCAAGGTGGAAATCAAA | | | | | |
| ADI-75758 | GACATCCAGATGACCCAGTCTCCATCCTCCCTGTCTGCATCTGTAGGAGACAGAGTCACCA | 3063 | DIQMTQSPSSLSASVGDRVTITCRASQR | 3345 | | |
| | TCACTTGCCGGGCAAGTCAGAGGATTAGTAGCTATTTAAATTGGTATCAGCAGAGACCAGG | | ISSYLNWYQQRPGKAPNFLIYNASSFES | | | |
| | GAAAGCCCCTAACTTCCTGATCTATAATGCATCCAGTTTTGAAAGTGGGGTCCCATCAAGG | | GVPSRFSGSGSGTDFTLTISSLQPEDFA | | | |
| | TTCAGTGGCAGTGGATCTGGGACAGATTTCACTCTCACCATCAGCAGTCTGCAACCTGAAG | | TYYCQQSYSDPYTFGQGTKVEIK | | | |
| | ATTTTGCAACTTACTACTGTCAACAGAGTTACAGTGACCCATACACTTTTGGCCAGGGGAC | | | | | |
| | CAAAGTGGAAATCAAA | | | | | |
| ADI-75793 | GACATCAAGATGACCCAGTCTCCATCCTTCCTGTCTGCATCTGTAGGAGACAGAGTCACCA | 3064 | DIKMTQSPSFLSASVGDRVTITCRASQG | 3346 | | |
| | TCACTTGCCGGGCCAGTCAGGGCATTAGCAGTTATTTAGCCTGGTATCAGCAAAAGCCAGG | | ISSYLAWYQQKPGKAPKLLIYAASTLQS | | | |
| | GAAAGCCCCTAAACTCCTAATCTATGCTGCATCCACTTTGCAAAGTGGGGTCCCATCAAGG | | GVPSRFSGSGSGTEFTLTISSLQPEDFA | | | |
| | TTCAGCGGCAGTGGATCTGGGACAGAATTCACTCTCACAATCAGCAGCCTGCAGCCTGAAG | | TYYCQQLNTHPPPFGPGTKVEIK | | | |
| | ATTTTGCAACTTATTACTGTCAACAGCTTAATACTCACCCTCCCCCTTTCGGCCCTGGGAC | | | | | |
| | CAAGGTGGAGATCAAA | | | | | |
| ADI-75804 | GACATCGTGATGACGCAGTCTCCATCCTCCCTGTCTGCATCTGTAGGAGACAGAGTCACCA | 3065 | DIVMTQSPSSLSASVGDRVTITCRASQS | 3347 | | |
| | TCACTTGCCGGGCAAGTCAGAGCATTAGCACGTATTTAAATTGGTATCAGCAGAAACCAGA | | ISTYLNWYQQKPEKAPKLLIYAASSLQS | | | |
| | GAAAGCCCCTAAGCTCCTGATCTATGCTGCATCCAGTTTGCAAAGTGGGGTCCCATCAAGG | | GVPSRFSGSGSGTDFTLTISSLQPEDFA | | | |
| | TTCAGTGGCAGCGGATCTGGGACAGATTTCACTCTCACCATCAGCAGTCTGCAACCTGAAG | | TYYCQQSYTSPAYTFGQGTKVEIK | | | |
| | ATTTTGCAACTTACTACTGTCAACAGAGTTACACTTCCCCTGCGTACACGTTTGGCCAGGG | | | | | |
| | GACCAAGGTGGAGATCAAA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75658 | IGKV1-9 | DIKMTQ | 1942 | RASQGIS | 2137 | WYQQKS | 2312 | GASTLQ | 2447 | GVPSRFSG | 2592 | QHLNS | 2790 | FGQGTR | 2940 |
| | | SPSFLS | | TDLA | | GKAPKL | | S | | SGSGTEFT | | DAIT | | LEIK | |
| | | ASVGDR | | | | LIY | | | | LTISSLQP | | | | | |
| | | VTITC | | | | | | | | EDFATYYC | | | | | |
| ADI-75768 | IGKV1-9 | DIQMTQ | 1943 | RASQGIS | 2138 | WYQQKP | 2257 | AASTLQ | 2392 | GVPSRFSG | 2593 | QHLDT | 2791 | FGRGTK | 2941 |
| | | SPSFLS | | RYLA | | GKAPKL | | S | | SGSGTEFT | | DSGT | | VEIK | |
| | | AFVGDR | | | | LIY | | | | LTISSLQT | | | | | |
| | | VTITC | | | | | | | | EDFATYYC | | | | | |
| ADI-75810 | IGKV4-1 | DIQMTQ | 1927 | KSNQSVL | 2139 | WYQQKP | 2290 | WASTRE | 2422 | GIPDRFSG | 2594 | QQYYN | 2732 | FGQGTK | 2937 |
| | | SPDSLA | | YSS-- | | GQPPKL | | S | | SGSGTDFT | | TPVT | | VEIK | |
| | | VSLGER | | KNFLG | | LIY | | | | LTISSLQA | | | | | |
| | | ATINC | | | | | | | | EDVAVYYC | | | | | |
| ADI-75784 | IGKV1-9 | DIKMTQ | 1944 | RASQGIS | 2140 | WYQQKP | 2257 | GASTLQ | 2447 | GVPLRFSG | 2595 | QHLDS | 2792 | FGQGTK | 2944 |
| | | TPSFLS | | SFLA | | GKAPKL | | S | | SGSGTEFT | | YPRYT | | VDIK | |
| | | ASVGDR | | | | LIY | | | | LTISSLQP | | | | | |
| | | VTITC | | | | | | | | EDFATYYC | | | | | |
| ADI-75619 | IGKV1- | EIQLTQ | 1917 | RASQGIS | 2062 | WYQQKP | 2259 | AASTLE | 2398 | GVPSRFSG | 2530 | QEYYD | 2793 | FGQGTK | 2944 |
| | NL1 | SPSSLS | | NSLA | | GKAPKL | | S | | SGSGTDFS | | TRT | | VDIK | |
| | | ASVGDR | | | | LLY | | | | LTISSLQP | | | | | |
| | | VTITC | | | | | | | | EDFATYYC | | | | | |
| ADI-75738 | IGKV3-11 | EIVLTQ | 1945 | RASQSVS | 2119 | WYQQRP | 2306 | DASNRA | 2448 | GIPARFSG | 2596 | QQRSN | 2794 | FGGGTK | 2936 |
| | | SPATLS | | TYLA | | GQAPRL | | T | | SGSETDFT | | WLT | | VEIK | |
| | | LSPGER | | | | LIY | | | | LTISSLEP | | | | | |
| | | ATLSC | | | | | | | | EDFAVYYC | | | | | |
| ADI-75790 | IGLV2-14 | QSALIQ | 1946 | GAYIYVS | 2141 | WYQQHP | 2313 | EVTKRP | 2449 | GISNRFSG | 2597 | SSYTT | 2795 | FGGGTQ | 2948 |
| | | PASVSG | | TATSSDV | | GKAPKL | | S | | SKSGNTAS | | SGVV | | LTVL | |
| | | SPGQSI | | | | TIY | | | | LTISGLQA | | | | | |
| | | TISC | | | | | | | | EDEAHYYC | | | | | |
| ADI-75719 | IGKV1D- | DIPMTQ | 1947 | RASQSIT | 2142 | WYQQKP | 2257 | AASSLQ | 2391 | GVPSRFSG | 2598 | QQSYN | 2702 | FGQGTK | 2937 |
| | 39 | SPSSLS | | TYLN | | GKAPKL | | S | | SGSGTDFT | | TPPWT | | VEIK | |
| | | ASVGDR | | | | LIY | | | | LTITSLQP | | | | | |
| | | VTITC | | | | | | | | EDFAIYYC | | | | | |
| ADI-75742 | IGLV1-40 | QSVLTQ | 1948 | GAGYDVH | 2143 | WYQQLP | 2314 | DNTNRP | 2450 | GVPDRFSG | 2599 | QSYDS | 2796 | FGTGTK | 2945 |
| | | PPSVSG | | TGSNSNI | | GTAPKL | | S | | SKSGTSAS | | SLTGS | | VTVL | |
| | | APGQRV | | | | LIY | | | | LAITGLQA | | V | | | |
| | | TISC | | | | | | | | EDEADYYC | | | | | |
| ADI-75779 | IGKV1-12 | EIPMTQ | 1949 | RASQGIG | 2144 | WYQQKP | 2257 | AASILQ | 2430 | GVPSRFSG | 2592 | QQADS | 2763 | FGPGTK | 2942 |
| | | SPSSVS | | SWLG | | GKAPKL | | S | | SGSGTEFT | | FPPA | | VEIK | |
| | | ASVGDR | | | | LIY | | | | LTISSLQP | | | | | |
| | | VTIAC | | | | | | | | EDFATYYC | | | | | |
| ADI-75650 | IGKV2-30 | EIVLTQ | 1950 | RSSESLS | 2145 | WFQQRP | 2315 | KVSNRD | 2451 | GVPDRFSG | 2533 | MQGTH | 2797 | FGQGTK | 2937 |
| | | SPLSLP | | YSDGNTY | | GQSPRR | | S | | SGSGTDFT | | WPPT | | VEIK | |
| | | VTLGQP | | LN | | LIY | | | | LKISRVEA | | | | | |
| | | ASISC | | | | | | | | EDVGVYYC | | | | | |
| ADI-75593 | IGKV1-5 | DIQMTQ | 1879 | RASQSID | 2126 | WYQQKP | 2257 | KTSTLE | 2452 | GVPSRFSG | 2532 | QHFNS | 2781 | FGQGTK | 2937 |
| | | SPSTLS | | DWLA | | GKAPKL | | S | | SGSGTEFT | | YLMYT | | VEIK | |
| | | ASVGDR | | | | LIY | | | | LTISSLQP | | | | | |
| | | VTITC | | | | | | | | DDFATYYC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75658 | GACATCAAGATGACCCAGTCTCCATCCTTCCTGTCTGCATCTGTAGGAGACAGAGTCACCA | 3066 | DIKMTQSPSFLSASVGDRVTITCRASQG | 3348 | | |
| | TCACTTGCCGGGCCAGTCAGGGCATTAGTACTGATTTAGCCTGGTATCAGCAAAAATCAGG | | ISTDLAWYQQKSGKAPKLLIYGASTLQS | | | |
| | GAAAGCCCCCAAGCTCCTGATCTATGGTGCATCCACTTTGCAAAGTGGGGTCCCATCAAGG | | GVPSRFSGSGSGTEFTLTISSLQPEDFA | | | |
| | TTCAGCGGCAGTGGATCTGGGACAGAATTCACTCTCACAATCAGCAGCCTGCAGCCTGAAG | | TYYCQHLNSDAITFGQGTRLEIK | | | |
| | ATTTTGCAACTTATTACTGTCAACACCTTAATAGTGACGCGATCACCTTCGGCCAAGGGAC | | | | | |
| | ACGACTGGAGATTAAA | | | | | |
| ADI-75768 | GACATCCAGATGACCCAGTCTCCATCCTTCCTGTCTGCATTTGTAGGAGACAGAGTCACCA | 3067 | DIQMTQSPSFLSAFVGDRVTITCRASQG | 3349 | | |
| | TCACTTGCCGGGCCAGTCAGGGCATTAGCAGGTATTTAGCCTGGTATCAGCAAAAACCAGG | | ISRYLAWYQQKPGKAPKLLIYAASTLQS | | | |
| | GAAAGCCCCTAAGCTCCTGATCTATGCTGCCTCCACTTTACAAAGTGGGGTCCCATCAAGG | | GVPSRFSGSGSGTEFTLTISSLQTEDFA | | | |
| | TTCAGCGGCAGTGGATCTGGGACAGAATTCACTCTCACAATCAGCAGCCTGCAGACTGAAG | | TYYCQHLDTDSGTFGRGTKVEIK | | | |
| | ATTTTGCAACTTATTACTGTCAACACCTTGATACTGACTCGGGGACGTTCGGCCGAGGGAC | | | | | |
| | CAAGGTGGAAATCAAA | | | | | |
| ADI-75810 | GACATCCAGATGACCCAGTCTCCAGACTCCCTGGCTGTGTCTCTGGGCGAGAGGGCCACCA | 3068 | DIQMTQSPDSLAVSLGERATINCKSNQS | 3350 | | |
| | TCAACTGCAAGTCCAACCAGAGTGTTTTATACAGTTCTAAGAACTTCTTAGGTTGGTACCA | | VLYSSKNFLGWYQQKPGQPPKLLIYWAS | | | |
| | GCAGAAACCAGGACAGCCTCCTAAACTGCTCATTTACTGGGCATCTACCCGGGAATCCGGG | | TRESGIPDRFSGSGSGTDFTLTISSLQA | | | |
| | ATCCCTGACCGATTCAGTGGCAGCGGCTCTGGGACAGATTTCACTCTCACCATCAGCAGCC | | EDVAVYYCQQYYNTPVTFGQGTKVEIK | | | |
| | TGCAGGCTGAAGATGTGGCAGTTTATTACTGTCAGCAATATTATAATACTCCTGTGACGTT | | | | | |
| | CGGCCAAGGGACCAAGGTGGAGATCAAA | | | | | |
| ADI-75784 | GACATCAAGATGACCCAGACTCCATCCTTCCTGTCTGCATCTGTGGGAGACAGAGTCACCA | 3069 | DIKMTQTPSFLSASVGDRVTITCRASQG | 3351 | | |
| | TCACTTGCCGGGCCAGTCAGGGCATTAGCAGTTTTTTAGCCTGGTATCAGCAAAAACCAGG | | ISSFLAWYQQKPGKAPKLLIYGASTLQS | | | |
| | GAAAGCCCCTAAGCTCCTGATCTATGGTGCATCCACTTTGCAAAGTGGGGTCCCATTAAGG | | GVPLRFSGSGSGTEFTLTISSLQPEDFA | | | |
| | TTCAGCGGCAGTGGATCTGGGACAGAATTCACTCTCACAATCAGCAGCCTGCAGCCTGAAG | | TYYCQHLDSYPRYTFGQGTKVDIK | | | |
| | ATTTTGCAACTTATTACTGTCAACACCTTGATAGTTACCCTCGGTACACTTTTGGCCAGGG | | | | | |
| | GACCAAGGTGGATATCAAA | | | | | |
| ADI-75619 | GAAATCCAATTGACCCAGTCTCCATCCTCCCTGTCTGCATCTGTCGGAGACAGAGTCACCA | 3070 | EIQLTQSPSSLSASVGDRVTITCRASQG | 3352 | | |
| | TCACTTGCCGGGCGAGTCAGGGCATTAGCAATTCTTTAGCCTGGTATCAGCAGAAACCAGG | | ISNSLAWYQQKPGKAPKLLLYAASTLES | | | |
| | GAAAGCCCCTAAGCTCCTGCTCTATGCTGCGTCTACGTTGGAAAGTGGGGTCCCATCCCGG | | GVPSRFSGSGSGTDFSLTISSLQPEDFA | | | |
| | TTCAGTGGGAGTGGATCTGGGACGGACTTCTCTCTCACCATCAGCAGCCTGCAGCCTGAGG | | TYYCQEYYDTRTFGQGTKVDIK | | | |
| | ATTTTGCAACTTATTACTGTCAAGAGTATTATGATACCCGGACGTTCGGCCAAGGGACCAA | | | | | |
| | GGTGGATATCAAA | | | | | |
| ADI-75738 | GAAATTGTGTTGACACAGTCTCCAGCCACCCTGTCTTTGTCTCCAGGGGAAAGAGCCACCC | 3071 | EIVLTQSPATLSLSPGERATLSCRASQS | 3353 | | |
| | TCTCCTGCAGGGCCAGTCAGAGTGTTAGCACCTACTTAGCCTGGTACCAACAGAGACCTGG | | VSTYLAWYQQRPGQAPRLLIYDASNRAT | | | |
| | CCAGGCTCCCAGGCTCCTCATCTATGATGCATCCAACAGGGCCACTGGCATCCCAGCCAGG | | GIPARFSGSGSETDFTLTISSLEPEDFA | | | |
| | TTCAGTGGCAGTGGGTCTGAGACAGACTTCACTCTCACCATCAGCAGCCTGGAGCCTGAAG | | VYYCQQRSNWLTFGGGTKVEIK | | | |
| | ATTTTGCAGTTTATTATTGTCAACAGCGGAGCAACTGGCTCACTTTCGGCGGGGGACCAA | | | | | |
| | GGTGGAAATCAAA | | | | | |
| ADI-75790 | CAGTCTGCTCTGATTCAGCCTGCCTCCGTGTCTGGGTCTCCTGGACAGTCGATCACCATCT | 3072 | QSALIQPASVSGSPGQSITISCTATSSD | 3354 | | |
| | CCTGCACTGCAACCAGTAGTGACGTTGGTGCTTATATCTATGTCTCGTGGTACCAACAACA | | VGAYIYVSWYQQHPGKAPKLTIYEVTKR | | | |
| | CCCAGGCAAAGCCCCCAAACTCACGATTTATGAGGTCACTAAACGGCCGTCAGGGATTTCT | | PSGISNRFSGSKSGNTASLTISGLQAED | | | |
| | AATCGCTTCTCTGGCTCCAAGTCTGGCAACACGGCCTCCCTGACCATCTCTGGGCTCCAGG | | EAHYYCSSYTTSGVVFGGGTQLTVL | | | |
| | CTGAGGACGAGGCTCATTATTACTGCAGCTCATATACAACTAGCGGCGTGGTATTCGGCGG | | | | | |
| | AGGCACCCAGCTGACCGTCCTA | | | | | |
| ADI-75719 | GACATCCCGATGACCCAGTCTCCATCCTCCCTGTCTGCATCTGTAGGAGACAGAGTCACCA | 3073 | DIPMTQSPSSLSASVGDRVTITCRASQS | 3355 | | |
| | TCACTTGCCGGGCAAGTCAGAGCATAACCACTTATTTAAATTGGTATCAGCAGAAACCAGG | | ITTYLNWYQQKPGKAPKLLIYAASSLQS | | | |
| | GAAAGCCCCTAAGCTCCTGATCTATGCTGCATCCAGTTTGCAAAGTGGGGTCCCATCAAGG | | GVPSRFSGSGSGTDFTLTITSLQPEDFA | | | |
| | TTCAGTGGCAGTGGATCTGGGACAGATTTCACTCTCACCATCACCAGTCTGCAACCTGAAG | | IYYCQQSYNTPPWTFGQGTKVEIK | | | |
| | ACTTTGCAATTTACTACTGTCAACAGAGTTACAATACCCCTCCGTGGACGTTCGGCCAAGG | | | | | |
| | GACCAAGGTGGAGATCAAA | | | | | |
| ADI-75742 | CAGTCTGTGTTGACGCAGCCGCCCTCAGTGTCTGGGGCCCCAGGGCAGAGGGTCACCATCT | 3074 | EADYYCQSYDSSLTGSVFGTGTKVTVL | 3356 | | |
| | CCTGCACTGGGAGCAACTCCAACATCGGGGCAGGTTATGATGTACACTGGTACCAGCAGCT | | QSVLTQPPSVSGAPGQRVTISCTGSNSN | | | |
| | TCCAGGAACAGCCCCCAAACTCCTCATCTATGATAACACCAATCGGCCCTCAGGGGTCCCT | | IGAGYDVHWYQQLPGTAPKLLIYDNTNR | | | |
| | GACCGATTCTCTGGGTCCAAGTCTGGCACCTCAGCCTCCCTGGCCATCACTGGGCTCCAGG | | PSGVPDRFSGSKSGTSASLAITGLQAED | | | |
| | CTGAGGATGAGGCTGATTATTACTGCCAGTCCTATGACAGCAGCCTGACTGGTTCTGTCTT | | | | | |
| | CGGAACTGGGACCAAGGTCACCGTCCTA | | | | | |
| ADI-75779 | GAAATCCCGATGACCCAGTCTCCATCTTCCGTGTCTGCATCTGTAGGAGACAGAGTCACCA | 3075 | EIPMTQSPSSVSASVGDRVTIACRASQG | 3357 | | |
| | TCGCTTGTCGGGCGAGTCAGGGTATTGGCAGCTGGTTAGGCTGGTATCAGCAGAAACCAGG | | IGSWLGWYQQKPGKAPKLLIYAASILQS | | | |
| | GAAAGCCCCTAAGCTCCTGATCTATGCTGCGTCCATTTTGCAAAGTGGAGTCCCATCAAGG | | GVPSRFSGSGSGTEFTLTISSLQPEDFA | | | |
| | TTCAGCGGCAGTGGATCTGGGACAGAATTCACTCTCACCATCAGCAGCCTGCAGCCTGAAG | | TYYCQQADSFPPAFGPGTKVEIK | | | |
| | ATTTTGCAACTTACTATTGTCAACAGGCTGACAGTTTCCCTCCGGCTTTCGGCCCTGGGAC | | | | | |
| | CAAGGTGGAAATCAAA | | | | | |
| ADI-75650 | GAAATTGTGTTGACACAGTCTCCGCTCTCCCTGCCCGTCACCCTTGGACAGCCGGCCTCCA | 3076 | EIVLTQSPLSLPVTLGQPASISCRSSES | 3358 | | |
| | TCTCCTGCAGGTCTAGTGAAAGCCTCTCATACAGTGATGGAAACACCTACTTGAATTGGTT | | LSYSDGNTYLNWFQQRPGQSPRRLIYKV | | | |
| | TCAGCAGAGGCCAGGCCAATCTCCAAGGCGCCTTATTTATAAGGTTTCTAACCGGGACTCT | | SNRDSGVPDRFSGSGSGTDFTLKISRVE | | | |
| | GGGGTCCCAGACAGATTCAGCGGCAGTGGGTCAGGCACTGATTTCACATTGAAAATCAGCA | | AEDVGVYYCMQGTHWPPTFGQGTKVEIK | | | |
| | GGGTGGAGGCTGAGGATGTTGGGGTTTATTACTGCATGCAAGGTACACACTGGCCTCCGAC | | | | | |
| | GTTCGGCCAAGGGACCAAGGTGGAAATCAAA | | | | | |
| ADI-75593 | GACATCCAGATGACCCAGTCTCCTTCCACCCTGTCTGCATCTGTGGGAGACAGAGTCACCA | 3077 | DIQMTQSPSTLSASVGDRVTITCRASQS | 3359 | | |
| | TCACTTGCCGGGCCAGTCAGAGTATTGATGACTGGTTGGCCTGGTATCAGCAGAAACCAGG | | IDDWLAWYQQKPGKAPKLLIYKTSTLES | | | |
| | GAAAGCCCCTAAACTCCTGATCTATAAGACGTCTACTTTAGAAAGTGGGGTCCCATCAAGA | | GVPSRFSGSGSGTEFTLTISSLQPDDFA | | | |
| | TTCAGCGGCAGTGGATCTGGGACAGAATTCACTCTCACCATCAGCAGCCTGCAGCCTGATG | | TYYCQHFNSYLMYTFGQGTKVEIK | | | |
| | ATTTTGCAACTTATTACTGCCAACACTTTAATAGTTACCTCATGTACACTTTTGGCCAGGG | | | | | |
| | GACCAAGGTGGAGATCAAA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75814 | IGKV1-5 | DIQMTQ | 1951 | RASQSIS | 2146 | WYQQKP | 2316 | KATHLE | 2453 | GVPSRFSG | 2532 | QQYNS | 2798 | FGQGTK | 2944 |
| | | SPSTLS | | TWLA | | GKAPKV | | T | | SGSGTEFT | | YYT | | VDIK | |
| | | ASVGDR | | | | LIY | | | | LTISSLQP | | | | | |
| | | VTVTC | | | | | | | | DDFATYYC | | | | | |
| ADI-75662 | IGLV3-21 | SYVLTQ | 1883 | GGNDLGS | 2147 | WYQQKP | 2287 | DDSDRP | 2399 | GIPERFSG | 2600 | QVWDS | 2742 | FGGGTK | 2939 |
| | | PPSVSV | | KNVH | | GQAPVL | | S | | SNSGNTAT | | NIDHP | | LTVL | |
| | | APGQTA | | | | VVF | | | | LTISRVET | | WV | | | |
| | | RITC | | | | | | | | GDEADYYC | | | | | |
| ADI-75627 | IGKV1-33 | DIQMTQ | 1878 | QASQDIN | 2148 | WYQQKP | 2257 | DASNLE | 2416 | GVPSRFSG | 2537 | QYYDN | 2799 | FGPGTK | 2942 |
| | | SPSSLS | | NYLN | | GKAPKL | | T | | SGSGTDFT | | PPPT | | VEIK | |
| | | ASVGDR | | | | LIY | | | | FTISSLQP | | | | | |
| | | VTITC | | | | | | | | EDIATYYC | | | | | |
| ADI-75792 | IGLV1-40 | QPVLTQ | 1952 | GGGYDVH | 2149 | WYQQLP | 2317 | LNNNRP | 2454 | GVPDRFSG | 2601 | QSYDS | 2800 | FGGGTQ | 2948 |
| | | PPSVSG | | TGSTSNI | | GEAPKL | | S | | SKSGTSAS | | SLSVW | | LTVL | |
| | | APGRGV | | | | LIY | | | | LAITGLQP | | V | | | |
| | | TISC | | | | | | | | VDEADYYC | | | | | |
| ADI-75791 | IGLV3-9 | QSVLTQ | 1953 | GLNIIPG | 2150 | WYQQKP | 2318 | RDNIRP | 2455 | GISERFSV | 2602 | QIWDI | 2801 | FGGGTQ | 2948 |
| | | PLSVSV | | KNVH | | GQAPIL | | S | | SHSGNMAT | | NTVI | | LTVL | |
| | | ALGQTA | | | | VIY | | | | LTISRAQA | | | | | |
| | | RIPC | | | | | | | | GDEADYYC | | | | | |
| ADI-75721 | IGLV3-21 | QYVLTQ | 1954 | GGNNIGS | 2151 | WYLQRP | 2319 | FNSDRP | 2456 | GIPGRFSG | 2603 | QVWDS | 2802 | FGGGTK | 2939 |
| | | PPSVSV | | KHVH | | GQAPVL | | S | | SNSGNTAT | | NTDHR | | LTVL | |
| | | APGETA | | | | VIS | | | | LTISRVEA | | V | | | |
| | | RITC | | | | | | | | GDEADYYC | | | | | |
| ADI-75761 | IGLV3-25 | DIRMTQ | 1955 | SGDALPN | 2152 | WYQQKS | 2320 | RDTERP | 2457 | GIPERFSG | 2604 | QSADY | 2803 | FGGGTK | 2939 |
| | | SPSVSV | | QYTY | | GQAPVL | | S | | SSSGTAVT | | SGTCV | | LTVL | |
| | | SPGQTA | | | | VIY | | | | LTISGVQA | | | | | |
| | | RITC | | | | | | | | EDEADYYC | | | | | |
| ADI-75682 | IGKV1D- | DIQLTQ | 1956 | RASQSIS | 2153 | WYQQKP | 2321 | AASSLQ | 2391 | GVPSRFSG | 2605 | QQSYS | 2804 | FGQGTR | 2940 |
| | 39 | SPSSLS | | TFLN | | GKVPNL | | S | | SGSGTDFT | | TPS | | LEIK | |
| | | ASVGDR | | | | LIY | | | | LTINGLQP | | | | | |
| | | VTVTC | | | | | | | | EDFATYYC | | | | | |
| ADI-75847 | IGKV1-9 | DIVLTQ | 1957 | RASQGIS | 2154 | WYQQKP | 2257 | AASTLQ | 2392 | GVPSRFSG | 2606 | QQLNS | 2805 | FGGGTK | 2938 |
| | | SPSSLS | | SHLA | | GKAPKL | | S | | SGSGTDFT | | DSPT | | VDIK | |
| | | ASVGDR | | | | LIY | | | | LTISSLQP | | | | | |
| | | VTITC | | | | | | | | EDFASYYC | | | | | |
| ADI-75852 | IGKV3-11 | EIALTQ | 1930 | RASQSVN | 2155 | WYQQKP | 2258 | DAYNRA | 2458 | GIPARFSG | 2607 | QQRLH | 2806 | FGPGTK | 2942 |
| | | SPATLS | | SYLA | | GQAPRL | | T | | SGSGTDFT | | PPFT | | VEIK | |
| | | LSPGER | | | | LIY | | | | LTISSLEP | | | | | |
| | | ATLSC | | | | | | | | EDFAVYYC | | | | | |
| ADI-75715 | IGKV1D- | DIQMTQ | 1878 | RASQSIS | 2156 | WYQQKP | 2257 | AASTLQ | 2392 | GVPSRFSG | 2608 | QQSYS | 2807 | FGQGTR | 2940 |
| | 39 | SPSSLS | | RFLS | | GKAPKL | | S | | SGSGTDFT | | TPPVT | | LEIK | |
| | | ASVGDR | | | | LIY | | | | LIISSLQP | | | | | |
| | | VTITC | | | | | | | | EDFATYFC | | | | | |
| ADI-75854 | IGLV3-25 | SYVLTQ | 1958 | SGDVLSK | 2157 | WYQQKP | 2304 | KDTERP | 2459 | GIPERLSG | 2609 | VSADS | 2808 | FGTGTK | 2949 |
| | | PPSVSV | | QYAY | | GQAPVL | | S | | SSSGTTVT | | SGVYV | | LTVL | |
| | | SPGQTA | | | | VIY | | | | LTISGVQA | | | | | |
| | | RITC | | | | | | | | EDEADYYC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75814 | GACATCCAGATGACCCAGTCTCCTTCCACCCTGTCTGCATCTGTTGGAGACAGAGTCACCG | 3078 | DIQMTQSPSTLSASVGDRVTVTCRASQS | 3360 | | |
| | TCACTTGCCGGGCCAGTCAGAGTATTAGTACCTGGTTGGCCTGGTATCAGCAGAAACCAGG | | ISTWLAWYQQKPGKAPKVLIYKATHLET | | | |
| | GAAAGCCCCTAAGGTCCTGATCTATAAGGCAACTCATTTAGAAACTGGGGTCCCATCAAGG | | GVPSRFSGSGSGTEFTLTISSLQPDDFA | | | |
| | TTCAGCGGCAGTGGATCTGGGACAGAATTCACTCTCACCATCAGCAGCCTGCAGCCTGATG | | TYYCQQYNSYYTFGQGTKVDIK | | | |
| | ATTTTGCAACTTATTACTGCCAACAGTATAATAGTTATTACACTTTTGGCCAGGGGACCAA | | | | | |
| | GGTGGATATCAAA | | | | | |
| ADI-75662 | TCGTATGTGCTGACTCAGCCACCCTCGGTGTCAGTGGCCCCAGGACAGACGGCCAGGATTA | 3079 | SYVLTQPPSVSVAPGQTARITCGGNDLG | 3361 | | |
| | CCTGTGGGGGAAACGACCTTGGAAGTAAAAATGTGCACTGGTACCAGCAGAAGCCAGGCCA | | SKNVHWYQQKPGQAPVLVVFDDSDRPSG | | | |
| | GGCCCCTGTGCTGGTCGTCTTTGATGATAGCGACCGGCCCTCAGGGATCCCTGAGCGATTC | | IPERFSGSNSGNTATLTISRVETGDEAD | | | |
| | TCTGGCTCCAACTCTGGGAACACGGCCACCCTGACCATCAGCAGGGTCGAAACCGGGGATG | | YYCQVWDSNIDHPWVFGGGTKLTVL | | | |
| | AGGCCGACTATTACTGTCAGGTGTGGGATAGTAATATTGATCATCCTTGGGTGTTCGGCGG | | | | | |
| | AGGGACCAAGCTCACCGTCCTA | | | | | |
| ADI-75627 | GACATCCAGATGACCCAGTCTCCATCCTCCCTGTCTGCATCTGTCGGAGACAGAGTCACCA | 3080 | DIQMTQSPSSLSASVGDRVTITCQASQD | 3362 | | |
| | TCACTTGCCAGGCGAGTCAGGACATTAACAACTATTTAAATTGGTATCAGCAGAAACCAGG | | INNYLNWYQQKPGKAPKLLIYDASNLET | | | |
| | AAAAGCCCCTAAGCTCCTCATCTACGATGCATCCAATTTGGAAACAGGGGTCCCATCAAGG | | GVPSRFSGSGSGTDFTFTISSLQPEDIA | | | |
| | TTCAGTGGAAGTGGATCTGGGACAGATTTTACTTTCACCATCAGCAGCCTGCAGCCTGAAG | | TYYCQYYDNPPPTFGPGTKVEIK | | | |
| | ATATTGCAACATATTACTGTCAATATTATGATAATCCCCCTCCGACTTTCGGCCCTGGGAC | | | | | |
| | CAAGGTGGAAATCAAA | | | | | |
| ADI-75792 | CAGCCTGTGCTGACTCAGCCGCCCTCAGTGTCTGGGGCCCCAGGGCGGGGGGTCACCATCT | 3081 | QPVLTQPPSVSGAPGRGVTISCTGSTSN | 3363 | | |
| | CCTGCACTGGGAGCACCTCCAACATCGGGGGAGGTTATGATGTGCACTGGTACCAGCAGCT | | IGGGYDVHWYQQLPGEAPKLLIYLNNNR | | | |
| | TCCAGGAGAGGCCCCCAAACTCCTCATCTATCTTAACAATAATCGGCCCTCAGGGGTCCCT | | PSGVPDRFSGSKSGTSASLAITGLQPVD | | | |
| | GACCGATTCTCTGGCTCCAAGTCTGGCACCTCAGCCTCCCTGGCCATCACTGGGCTCCAGC | | EADYYCQSYDSSLSVWVFGGGTQLTVL | | | |
| | CTGTCGATGAGGCTGATTATTACTGCCAGTCGTATGACAGCAGCCTGAGTGTTTGGGTGTT | | | | | |
| | CGGCGGAGGCACCCAGCTCACCGTCCTA | | | | | |
| ADI-75791 | CAGTCTGTGCTGACTCAGCCCCTCTCAGTGTCAGTGGCCCTGGGACAGACGGCCAGGATTC | 3082 | QSVLTQPLSVSVALGQTARIPCGLNIIP | 3364 | | |
| | CCTGTGGACTGAACATCATTCCTGGTAAAAATGTGCACTGGTACCAGCAGAAGCCAGGCCA | | GKNVHWYQQKPGQAPILVIYRDNIRPSG | | | |
| | GGCCCCTATACTGGTCATCTATAGGGATAACATCCGGCCCTCTGGAATCTCTGAGCGATTC | | ISERFSVSHSGNMATLTISRAQAGDEAD | | | |
| | TCTGTCTCCCACTCGGGGAACATGGCCACCCTGACCATCAGTAGAGCCCAAGCCGGGGATG | | YYCQIWDINTVIFGGGTQLTVL | | | |
| | AGGCTGACTATTACTGTCAGATTTGGGACATCAACACTGTGATATTCGGCGGAGGGACCCA | | | | | |
| | GCTGACCGTCCTA | | | | | |
| ADI-75721 | GACATCCGGATGACCCAGTCTCCCTCGGTGTCAGTGTCCCCGGGACAGACGGCCAGGATCA | 3083 | QYVLTQPPSVSVAPGETARITCGGNNIG | 3365 | | |
| | CCTGCTCTGGAGATGCATTGCCAAATCAATATACTTATTGGTACCAACAAAAGTCAGGCCA | | SKHVHWYLQRPGQAPVLVISFNSDRPSG | | | |
| | GGCCCCTGTGTTGGTGATATATAGAGACACTGAGAGGCCCTCAGGGATCCCTGAGCGATTC | | IPGRFSGSNSGNTATLTISRVEAGDEAD | | | |
| | TCTGGCTCCAGCTCAGGGACGGCAGTCACGTTGACCATCAGTGGAGTCCAGGCAGAAGACG | | YYCQVWDSNTDHRVFGGGTKLTVL | | | |
| | AGGCTGACTATTACTGTCAATCAGCAGACTATAGCGGTACTTGCGTGTTCGGCGGGGGGAC | | | | | |
| | CAAGCTGACCGTCCTA | | | | | |
| ADI-75761 | GACATCCGGATGACCCAGTCTCCCTCGGTGTCAGTGTCCCCGGGACAGACGGCCAGGATCA | 3084 | DIRMTQSPSVSVSPGQTARITCSGDALP | 3366 | | |
| | CCTGCTCTGGAGATGCATTGCCAAATCAATATACTTATTGGTACCAACAAAAGTCAGGCCA | | NQYTYWYQQKSGQAPVLVIYRDTERPSG | | | |
| | GGCCCCTGTGTTGGTGATATATAGAGACACTGAGAGGCCCTCAGGGATCCCTGAGCGATTC | | IPERFSGSSSGTAVTLTISGVQAEDEAD | | | |
| | TCTGGCTCCAGCTCAGGGACGGCAGTCACGTTGACCATCAGTGGAGTCCAGGCAGAAGACG | | YYCQSADYSGTCVFGGGTKLTVL | | | |
| | AGGCTGACTATTACTGTCAATCAGCAGACTATAGCGGTACTTGCGTGTTCGGCGGGGGGAC | | | | | |
| | CAAGCTGACCGTCCTA | | | | | |
| ADI-75682 | GACATCCAGTTGACCCAGTCTCCATCCTCCCTGTCTGCATCTGTAGGAGACAGAGTCACCG | 3085 | DIQLTQSPSSLSASVGDRVTVTCRASQS | 3367 | | |
| | TCACTTGCCGGGCAAGTCAGAGCATTAGCACCTTTTTAAATTGGTATCAACAGAAACCAGG | | ISTFLNWYQQKPGKVPNLLIYAASSLQS | | | |
| | GAAAGTCCCTAACCTCCTGATCTATGCTGCATCCAGCTTGCAAAGTGGGGTCCCATCAAGG | | GVPSRFSGSGSGTDFTLTINGLQPEDFA | | | |
| | TTCAGTGGGAGTGGATCTGGGACAGATTTCACTCTCACCATCAACGGTCTGCAACCTGAAG | | TYYCQQSYSTPSFGQGTRLEIK | | | |
| | ATTTTGCAACTTACTACTGTCAGCAGAGTTACAGTACCCCTTCCTTCGGCCAAGGGACACG | | | | | |
| | ACTGGAGATTAAA | | | | | |
| ADI-75847 | GACATTGTGTTGACCCAGTCTCCATCCTCCCTGTCTGCATCTGTAGGAGACAGAGTCACCA | 3086 | DIVLTQSPSSLSASVGDRVTITCRASQG | 3368 | | |
| | TCACTTGCCGGGCCAGTCAGGGCATTAGCAGTCATTTAGCCTGGTATCAGCAAAAACCAGG | | ISSHLAWYQQKPGKAPKLLIYAASTLQS | | | |
| | GAAAGCCCCTAAGCTTCTGATCTATGCTGCATCCACTTTGCAAAGTGGGGTCCCATCAAGG | | GVPSRFSGSGSGTDFTLTISSLQPEDFA | | | |
| | TTCAGCGGCAGTGGATCTGGGACAGATTTCACTCTCACCATCAGCAGCCTGCAGCCTGAAG | | SYYCQQLNSDSPTFGGGTKVDIK | | | |
| | ATTTTGCAAGTTATTACTGTCAACAGCTTAATAGTGACTCCCCCACTTTCGGCGGAGGGAC | | | | | |
| | CAAAGTGGATATCAAA | | | | | |
| ADI-75852 | GAAATTGCACTGACGCAGTCTCCAGCCACCCTGTCTTTGTCTCCAGGGGAAAGAGCCACCC | 3087 | EIALTQSPATLSLSPGERATLSCRASQS | 3369 | | |
| | TCTCCTGCAGGGCCAGTCAGAGTGTTAACAGCTACTTAGCCTGGTACCAACAGAAACCTGG | | VNSYLAWYQQKPGQAPRLLIYDAYNRAT | | | |
| | CCAGGCTCCCAGGCTCCTCATCTATGATGCATACAACAGGGCCACTGGCATCCCAGCCAGG | | GIPARFSGSGSGTDFTLTISSLEPEDFA | | | |
| | TTCAGTGGCAGTGGGTCTGGGACAGACTTCACTCTCACCATCAGCAGCCTAGAGCCTGAGG | | VYYCQQRLHPPFTFGPGTKVEIK | | | |
| | ATTTTGCAGTTTATTACTGTCAGCAACGTCTCCACCCGCCATTCACTTTCGGCCCTGGGAC | | | | | |
| | CAAGGTGGAGATCAAA | | | | | |
| ADI-75715 | GACATCCAGATGACCCAGTCTCCATCCTCCCTGTCTGCATCTGTAGGAGACAGAGTCACCA | 3088 | DIQMTQSPSSLSASVGDRVTITCRASQS | 3370 | | |
| | TCACTTGCCGGGCAAGTCAGAGCATTAGTAGATTTTTAAGTTGGTATCAGCAGAAACCAGG | | ISRFLSWYQQKPGKAPKLLIYAASTLQS | | | |
| | GAAAGCCCCTAAGCTCCTGATCTATGCTGCATCCACTTTGCAGAGTGGGGTCCCATCAAGG | | GVPSRFSGSGSGTDFTLIISSLQPEDFA | | | |
| | TTCAGTGGCAGTGGATCTGGGACAGATTTCACTCTCATTATCAGCAGTCTGCAACCTGAAG | | TYFCQQSYSTPPVTFGQGTRLEIK | | | |
| | ATTTTGCAACCTACTTCTGTCAACAGAGTTACAGTACCCCTCCGGTCACCTTCGGCCAAGG | | | | | |
| | GACACGACTGGAGATTAAA | | | | | |
| ADI-75854 | TCCTATGTGCTGACACAGCCACCCTCGGTGTCAGTGTCCCCAGGACAGACGGCCAGGATCA | 3089 | SYVLTQPPSVSVSPGQTARITCSGDVLS | 3371 | | |
| | CCTGCTCCGGAGATGTATTGTCAAAGCAATACGCTTATTGGTACCAACAGAAACCAGGCCA | | KQYAYWYQQKPGQAPVLVIYKDTERPSG | | | |
| | GGCCCCTGTGTTGGTGATATATAAAGACACTGAGAGGCCCTCAGGGATCCCTGAGCGATTG | | IPERLSGSSSGTTVTLTISGVQAEDEAD | | | |
| | TCTGGCTCCAGCTCAGGGACAACAGTCACGTTGACCATCAGTGGAGTCCAGGCAGAAGACG | | YYCVSADSSGVYVFGTGTKLTVL | | | |
| | AGGCTGACTATTATTGTGTATCAGCAGACAGTAGTGGTGTTTATGTCTTCGGAACTGGGAC | | | | | |
| | CAAGCTGACCGTCCTA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75701 | IGLV1-40 | QSVLTQ | 1948 | TGSSSNI | 2158 | WYQQIP | 2322 | ANDNRP | 2460 | GVPDRFSG | 2599 | QSYDS | 2809 | FGGGTK | 2939 |
| | | PPSVSG | | GAGHDVN | | GTAPKL | | S | | SKSGTSAS | | GLSGS | | LTVL | |
| | | APGQRV | | | | LIS | | | | LAITGLQA | | VL | | | |
| | | TISC | | | | | | | | EDEADYYC | | | | | |
| ADI-75681 | IGKV1-33 | DIQMTQ | 1878 | QASQDIN | 2159 | WYQQKP | 2323 | DASNLE | 2461 | GVPSRFSG | 2610 | QQYDN | 2810 | FGPGTK | 2942 |
| | | SPSSLS | | KFLN | | GKAPTL | | P | | SGSGTDFT | | VPPT | | VEIK | |
| | | ASVGDR | | | | LIY | | | | FTITSLQP | | | | | |
| | | VTITC | | | | | | | | EDIATYYC | | | | | |
| ADI-75757 | IGKV1-33 | EIQMTQ | 1881 | QASQDIN | 2160 | WYQQKP | 2316 | DASNLE | 2416 | GVPSRFSG | 2611 | QQYDN | 2811 | FGGGTK | 2936 |
| | | SPSSLS | | KYLN | | GKAPKV | | T | | SGSGTDFT | | LPQT | | VEIK | |
| | | ASVGDR | | | | LIY | | | | FTISGLQP | | | | | |
| | | VTITC | | | | | | | | EDIATYYC | | | | | |
| ADI-75656 | IGKV1-5 | TCVMTQ | 1959 | RTSQSIS | 2161 | WYQQKP | 2257 | KASSLE | 2386 | GVPPRFSG | 2612 | QQYKS | 2812 | FGQGTK | 2937 |
| | | SPSTLS | | TWLA | | GKAPKL | | S | | SGSGTEFT | | FWT | | VEIK | |
| | | ASVGDR | | | | LIY | | | | LTISSLQP | | | | | |
| | | VTITC | | | | | | | | DDFATYYC | | | | | |
| ADI-75677 | IGKV1-5 | DIVMTQ | 1960 | RASQSIS | 2146 | WYQQKP | 2257 | KASSLE | 2386 | GVPSRFSG | 2532 | QQYNS | 2772 | FGQGTK | 2944 |
| | | TPPTLS | | TWLA | | GKAPKL | | S | | SGSGTEFT | | YWT | | VDIK | |
| | | ASVGDR | | | | LIY | | | | LTISSLQP | | | | | |
| | | VTITC | | | | | | | | DDFATYYC | | | | | |
| ADI-75860 | IGLV1-44 | QSVLTQ | 1961 | SGSSSNI | 2162 | WYQQFP | 2324 | TNNQRR | 2462 | GVPDRFSG | 2613 | AAWDD | 2813 | FGGGTK | 2939 |
| | | PPSASG | | GSNSVN | | GTAPKL | | PS | | SKSGTSAS | | SLNGV | | LTVL | |
| | | TPGQRV | | | | LIY | | | | LAISDLQS | | V | | | |
| | | VISC | | | | | | | | EDEADYHC | | | | | |
| ADI-75672 | IGKV1D- | EIQMTQ | 1881 | RASQSIS | 2050 | WYQQKP | 2325 | AASSLQ | 2391 | GVPSRFSG | 2614 | QQSYS | 2814 | FGQGTK | 2937 |
| | 39 | SPSSLS | | SYLN | | GKAPKL | | S | | SGSGTDFT | | LPEYT | | VEIK | |
| | | ASVGDR | | | | LMY | | | | LTISNLQP | | | | | |
| | | VTITC | | | | | | | | EDFATYYC | | | | | |
| ADI-75684 | IGLV1-44 | SYELTQ | 1962 | SGSSSNI | 2162 | WYQQLP | 2314 | SNNQRP | 2463 | GVPDRFSG | 2615 | ATWDD | 2815 | FGGGTQ | 2953 |
| | | PPSASG | | GSNSVN | | GTAPKL | | S | | SKSGTSAS | | SLTGP | | VTVL | |
| | | TPGQRV | | | | LIY | | | | LAISGLQS | | V | | | |
| | | TISC | | | | | | | | EDEADYYC | | | | | |
| ADI-75733 | IGKV3-20 | EMTLTQ | 1963 | RASQSV- | 2163 | WYQQKP | 2258 | GASSRA | 2464 | GIPDRFSG | 2616 | QQYGS | 2780 | FGQGTK | 2937 |
| | | SPGTLS | | SSYLA | | GQAPRL | | T | | SGSGTDFT | | SPRT | | VEIK | |
| | | LSPGER | | | | LIY | | | | LTISRLEP | | | | | |
| | | ATLSC | | | | | | | | EDFAVYYC | | | | | |
| ADI-75732 | IGKV1D- | DIVMTQ | 1902 | RANQTIS | 2164 | WYQQKP | 2257 | AASSLQ | 2391 | GVPSRFSG | 2617 | QQSYS | 2816 | FGLGTK | 2954 |
| | 39 | SPSSLS | | NYLN | | GKAPKL | | S | | SGSGTDFT | | SPPT | | VEIK | |
| | | ASVGDR | | | | LIY | | | | LTISSLQP | | | | | |
| | | VTITC | | | | | | | | EDFAIYFC | | | | | |
| ADI-75722 | IGLV2-14 | SLLLTQ | 1964 | TGASSDV | 2165 | WYQQHP | 2265 | EVTNRP | 2465 | GVSDRFSG | 2618 | SSYTS | 2817 | FGGGTK | 2939 |
| | | PASVSG | | GAYIYVS | | GKAPKL | | S | | SKSGNTAS | | SSAV | | LTVL | |
| | | SPGQSI | | | | MIY | | | | LTISGLQA | | | | | |
| | | TISC | | | | | | | | EDEADYYC | | | | | |
| ADI-75674 | IGKV4-1 | DIQMTQ | 1965 | MSSQSVL | 2166 | WYQQKP | 2326 | WASTRK | 2436 | GVPDRFSG | 2619 | QQYYR | 2771 | FGGGTK | 2936 |
| | | SPDSLA | | DSSNDKN | | GQPPKL | | S | | SGSGTDFT | | TPLT | | VEIK | |
| | | VSLGER | | FLA | | FIY | | | | LTISSLQA | | | | | |
| | | ATIKC | | | | | | | | EDVAVYYC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75701 | CAGTCTGTGTTGACGCAGCCGCCCTCAGTGTCTGGAGCCCCAGGGCAGAGGGTCACCATCT | 3090 | QSVLTQPPSVSGAPGQRVTISCTGSSSN | 3372 | | |
| | CCTGCACTGGGAGCAGCTCCAACATCGGGGCAGGTCATGATGTGAATTGGTACCAGCAAAT | | IGAGHDVNWYQQIPGTAPKLLISANDNR | | | |
| | TCCAGGAACAGCCCCCAAACTCCTCATCTCTGCTAACGACAACCGGCCCTCAGGGGTCCCT | | PSGVPDRFSGSKSGTSASLAITGLQAED | | | |
| | GACCGATTCTCTGGCTCCAAGTCTGGCACCTCAGCCTCCCTGGCCATCACTGGGCTCCAGG | | EADYYCQSYDSGLSGSVLFGGGTKLTVL | | | |
| | CTGAGGATGAGGCTGATTATTACTGCCAGTCCTATGACAGCGGACTGAGTGGTTCTGTGCT | | | | | |
| | ATTCGGCGGAGGGACCAAGCTGACCGTCCTA | | | | | |
| ADI-75681 | GACATCCAGATGACCCAGTCTCCATCCTCCCTGTCTGCATCTGTAGGAGACAGAGTCACCA | 3091 | DIQMTQSPSSLSASVGDRVTITCQASQD | 3373 | | |
| | TCACTTGCCAGGCGAGTCAGGACATTAACAAGTTTTTAAATTGGTATCAGCAGAAACCAGG | | INKFLNWYQQKPGKAPTLLIYDASNLEP | | | |
| | GAAAGCCCCTACGCTCCTGATCTACGATGCATCCAATTTGGAACCAGGGGTCCCATCAAGG | | GVPSRFSGSGSGTDFTFTITSLQPEDIA | | | |
| | TTCAGTGGAAGTGGATCTGGGACAGATTTTACTTTCACCATCACCAGCCTGCAGCCTGAAG | | TYYCQQYDNVPPTFGPGTKVEIK | | | |
| | ATATTGCAACATATTACTGTCAACAGTATGATAATGTTCCTCCGACTTTCGGCCCTGGGAC | | | | | |
| | CAAGGTGGAAATCAAA | | | | | |
| ADI-75757 | GAAATCCAAATGACCCAGTCTCCATCCTCCCTGTCTGCATCTGTAGGAGACAGAGTCACCA | 3092 | EIQMTQSPSSLSASVGDRVTITCQASQD | 3374 | | |
| | TCACTTGCCAGGCGAGTCAGGACATTAACAAGTATTTAAATTGGTATCAGCAGAAACCAGG | | INKYLNWYQQKPGKAPKVLIYDASNLET | | | |
| | GAAAGCCCCTAAGGTCCTGATCTACGATGCATCCAATTTGGAAACAGGAGTCCCATCAAGG | | GVPSRFSGSGSGTDFTFTISGLQPEDIA | | | |
| | TTCAGTGGAAGTGGATCTGGGACAGATTTTACTTTCACCATCAGCGGCCTGCAGCCTGAAG | | TYYCQQYDNLPQTFGGGTKVEIK | | | |
| | ATATTGCAACATATTACTGTCAACAGTATGATAATCTCCCTCAGACTTTCGGCGGAGGGAC | | | | | |
| | CAAGGTGGAAATCAAA | | | | | |
| ADI-75656 | ACATGCGTGATGACGCAGTCTCCTTCCACCCTGTCTGCATCTGTAGGAGACAGAGTCACCA | 3093 | TCVMTQSPSTLSASVGDRVTITCRTSQS | 3375 | | |
| | TCACTTGCCGGACCAGTCAGAGTATTAGTACCTGGTTGGCCTGGTATCAGCAGAAACCAGG | | ISTWLAWYQQKPGKAPKLLIYKASSLES | | | |
| | GAAAGCCCCTAAGCTCCTGATCTATAAGGCGTCTAGTTTAGAAAGTGGGGTCCCACCAAGG | | GVPPRFSGSGSGTEFTLTISSLQPDDFA | | | |
| | TTCAGCGGCAGTGGATCTGGGACAGAATTCACTCTCACCATCAGCAGCCTGCAGCCTGATG | | TYYCQQYKSFWTFGQGTKVEIK | | | |
| | ATTTTGCTACTTATTACTGCCAGCAGTATAAAAGTTTTTGGACGTTCGGCCAAGGGACCAA | | | | | |
| | GGTGGAAATCAAA | | | | | |
| ADI-75677 | GACATCGTGATGACCCAGACTCCTCCCACCCTGTCTGCATCTGTAGGAGACAGAGTCACCA | 3094 | DIVMTQTPPTLSASVGDRVTITCRASQS | 3376 | | |
| | TCACTTGCCGGGCCAGTCAGAGTATTAGTACCTGGTTGGCCTGGTATCAGCAGAAACCAGG | | ISTWLAWYQQKPGKAPKLLIYKASSLES | | | |
| | GAAAGCCCCTAAGCTCCTGATCTATAAGGCGTCTAGTTTAGAGAGTGGGGTCCCATCAAGG | | GVPSRFSGSGSGTEFTLTISSLQPDDFA | | | |
| | TTCAGCGGCAGTGGATCTGGGACAGAGTTCACTCTCACCATCAGCAGCCTGCAGCCTGATG | | TYYCQQYNSYWTFGQGTKVDIK | | | |
| | ATTTTGCAACTTATTACTGCCAACAGTATAATAGTTATTGGACGTTCGGCCAAGGGACCAA | | | | | |
| | GGTGGATATCAAA | | | | | |
| ADI-75860 | CAGTCTGTGTTGACGCAGCCGCCCTCAGCGTCTGGGACCCCCGGGCAGAGGGTCGTCATTT | 3095 | QSVLTQPPSASGTPGQRVVISCSGSSSN | 3377 | | |
| | CTTGTTCTGGGAGCAGCTCCAACATCGGAAGTAATAGTGTAAACTGGTACCAGCAATTTCC | | IGSNSVNWYQQFPGTAPKLLIYTNNQRR | | | |
| | AGGAACGGCCCCCAAACTCCTCATCTATACTAATAATCAGCGGCGGCCCTCAGGGGTCCCT | | PSGVPDRFSGSKSGTSASLAISDLQSED | | | |
| | GACCGATTCTCTGGCTCCAAGTCTGGCACCTCAGCCTCCCTGGCCATCAGTGACCTCCAGT | | EADYHCAAWDDSLNGVVFGGGTKLTVL | | | |
| | CTGAGGATGAGGCTGATTATCACTGTGCAGCATGGGATGACAGCCTGAATGGTGTGGTCTT | | | | | |
| | CGGCGGAGGGACCAAGCTCACCGTCCTA | | | | | |
| ADI-75672 | GAAATCCAGATGACCCAGTCTCCATCCTCCCTGTCTGCATCTGTAGGAGACAGAGTCACCA | 3096 | EIQMTQSPSSLSASVGDRVTITCRASQS | 3378 | | |
| | TCACTTGCCGGGCAAGTCAGAGCATTAGTAGTTATTTAAATTGGTATCAGCAGAAACCAGG | | ISSYLNWYQQKPGKAPKLLMYAASSLQS | | | |
| | GAAAGCCCCTAAGCTCCTGATGTATGCTGCATCCAGTTTGCAAAGTGGGGTCCCATCAAGG | | GVPSRFSGSGSGTDFTLTISNLQPEDFA | | | |
| | TTCAGTGGCAGTGGATCTGGGACAGATTTCACTCTCACCATCAGCAATCTGCAACCTGAAG | | TYYCQQSYSLPEYTFGQGTKVEIK | | | |
| | ATTTTGCAACCTACTACTGTCAACAGAGTTACAGTTTGCCCGAGTACACTTTTGGCCAGGG | | | | | |
| | GACCAAGGTGGAGATCAAA | | | | | |
| ADI-75684 | TCGTATGAGCTGACACAGCCACCCTCAGCGTCTGGGACCCCCGGGCAGAGGGTCACCATCT | 3097 | SYELTQPPSASGTPGQRVTISCSGSSSN | 3379 | | |
| | CTTGTTCTGGAAGCAGCTCCAACATCGGAAGTAATTCTGTAAACTGGTACCAGCAACTCCC | | IGSNSVNWYQQLPGTAPKLLIYSNNQRP | | | |
| | AGGAACGGCCCCCAAACTCCTCATCTATAGTAATAATCAGCGGCCCTCAGGGGTCCCTGAC | | SGVPDRFSGSKSGTSASLAISGLQSEDE | | | |
| | CGATTCTCTGGCTCCAAGTCTGGCACCTCAGCCTCCCTGGCCATCAGTGGGCTCCAGTCTG | | ADYYCATWDDSLTGPVFGGGTQVTVL | | | |
| | AGGATGAGGCTGATTATTACTGCGCAACATGGGATGACAGCCTGACTGGTCCGGTGTTCGG | | | | | |
| | CGGAGGGACCCAGGTGACCGTCCTC | | | | | |
| ADI-75733 | GAAATGACACTGACGCAGTCTCCAGGCACCCTGTCTTTGTCTCCAGGGGAAAGAGCCACCC | 3098 | EMTLTQSPGTLSLSPGERATLSCRASQS | 3380 | | |
| | TCTCCTGCAGGGCCAGTCAGAGTGTTAGCAGCTACTTAGCCTGGTACCAACAGAAACCTGG | | VSSYLAWYQQKPGQAPRLLIYGASSRAT | | | |
| | CCAGGCTCCCAGGCTCCTCATCTATGGTGCATCCAGCAGGGCCACTGGCATCCCAGACAGG | | GIPDRFSGSGSGTDFTLTISRLEPEDFA | | | |
| | TTCAGTGGCAGTGGGTCTGGGACAGACTTCACTCTCACCATCAGCAGACTGGAGCCTGAAG | | VYYCQQYGSSPRTFGQGTKVEIK | | | |
| | ATTTTGCAGTGTATTACTGTCAGCAGTATGGTAGCTCACCGAGGACGTTCGGCCAAGGGAC | | | | | |
| | CAAGGTGGAGATCAAA | | | | | |
| ADI-75732 | GACATCGTGATGACCCAGTCTCCATCTTCCCTGTCTGCATCTGTGGGAGACAGAGTCACCA | 3099 | DIVMTQSPSSLSASVGDRVTITCRANQT | 3381 | | |
| | TCACTTGCCGGGCAAACCAGACCATTAGCAACTATTTAAATTGGTATCAGCAGAAACCAGG | | ISNYLNWYQQKPGKAPKLLIYAASSLQS | | | |
| | GAAAGCCCCTAAACTCCTGATCTATGCTGCATCCAGTTTGCAAAGTGGGGTCCCGTCAAGG | | GVPSRFSGSGSGTDFTLTISSLQPEDFA | | | |
| | TTCAGTGGCAGTGGATCTGGGACAGATTTCACTCTCACCATCAGCAGTCTGCAACCTGAAG | | IYFCQQSYSSPPTFGLGTKVEIK | | | |
| | ATTTTGCAATTTATTTCTGTCAACAGAGTTACAGTTCCCCTCCGACGTTCGGCCTAGGGAC | | | | | |
| | CAAGGTGGAAATCAAA | | | | | |
| ADI-75722 | AGCCTGCTCCTGACTCAGCCTGCCTCCGTGTCTGGGTCTCCTGGACAGTCGATCACCATCT | 3100 | SLLLTQPASVSGSPGQSITISCTGASSD | 3382 | | |
| | CCTGCACTGGAGCCAGCAGTGACGTTGGTGCTTATATCTATGTCTCCTGGTACCAACAGCA | | VGAYIYVSWYQQHPGKAPKLMIYEVTNR | | | |
| | CCCAGGCAAAGCCCCCAAACTCATGATTTATGAGGTCACTAATCGGCCCTCAGGGGTTTCT | | PSGVSDRFSGSKSGNTASLTISGLQAED | | | |
| | GATCGCTTCTCTGGCTCCAAGTCTGGCAACACGGCCTCCCTGACCATCTCTGGGCTCCAGG | | EADYYCSSYTSSSAVFGGGTKLTVL | | | |
| | CTGAGGACGAGGCTGATTATTACTGCAGCTCATATACAAGCAGCAGCGCGGTATTCGGCGG | | | | | |
| | AGGGACCAAGCTCACCGTCCTA | | | | | |
| ADI-75674 | GACATCCAGATGACCCAGTCTCCAGACTCCCTGGCTGTGTCTCTGGGCGAGAGGGCCACCA | 3101 | DIQMTQSPDSLAVSLGERATIKCMSSQS | 3383 | | |
| | TCAAGTGCATGTCCAGCCAGAGTGTCTTAGACAGCTCCAACGATAAGAACTTCTTAGCTTG | | VLDSSNDKNFLAWYQQKPGQPPKLFIYW | | | |
| | GTATCAGCAGAAACCAGGACAGCCTCCTAAGTTGTTCATTTACTGGGCATCTACCCGGAAA | | ASTRKSGVPDRFSGSGSGTDFTLTISSL | | | |
| | TCCGGGGTCCCTGACCGATTCAGTGGCAGCGGGTCCGGGACAGATTTCACTCTCACAATCA | | QAEDVAVYYCQQYYRTPLTFGGGTKVEI | | | |
| | GCAGCCTGCAGGCTGAAGATGTGGCAGTTTATTACTGTCAGCAATATTATAGAACTCCTCT | | K | | | |
| | CACTTTCGGCGGAGGGACCAAGGTGGAAATCAAA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75638 | IGKV1-33 | DIQMTQ | 1966 | QASQDIN | 2167 | WYQQKA | 2327 | DASILE | 2466 | GVPSRFSG | 2620 | QQFDN | 2818 | FGQGTK | 2944 |
| | | SPSSLS | | KYLA | | GRAPKL | | T | | SGSGTHFT | | VPYT | | VDIK | |
| | | ASVGDR | | | | LIY | | | | LTINSLQT | | | | | |
| | | VAITC | | | | | | | | EDIATYYC | | | | | |
| ADI-75800 | IGKV3-11 | DIQVTQ | 1967 | RASQSVS | 2168 | WYQHKP | 2328 | DASNRA | 2448 | GIPARFSG | 2607 | QLRSN | 2819 | FGPGTK | 2942 |
| | | SPATLS | | SYLA | | GQAPRL | | T | | SGSGTDFT | | WPPFT | | VEIK | |
| | | LSPGER | | | | LIY | | | | LTISSLEP | | | | | |
| | | ATLSC | | | | | | | | EDFAVYYC | | | | | |
| ADI-75675 | IGKV1D- | DIVMTQ | 1968 | RASQTVR | 2169 | WYQQKP | 2288 | TAPTVE | 2467 | GVPPRFSA | 2621 | QQSYS | 2820 | FGQGTK | 2955 |
| | 39 | SPSSLS | | SFLN | | GKAPKL | | G | | TGSGTDFT | | IPYT | | LEIK | |
| | | ASVGDR | | | | LVF | | | | LSISGLQP | | | | | |
| | | VSITC | | | | | | | | EDFATYFC | | | | | |
| ADI-75818 | IGKV1-33 | DIQMTQ | 1878 | QASQDIS | 2170 | WYQQKP | 2257 | DALNLK | 2468 | GVPSRFSG | 2537 | QQYNN | 2821 | FGQGTK | 2937 |
| | | SPSSLS | | DYLN | | GKAPKL | | S | | SGSGTDFT | | FPYT | | VEIK | |
| | | ASVGDR | | | | LIY | | | | FTISSLQP | | | | | |
| | | VTITC | | | | | | | | EDIATYYC | | | | | |
| ADI-75741 | IGLV1-40 | QSVVTQ | 1969 | GAGYDVH | 2171 | WYQQLP | 2314 | DNSNRP | 2469 | GVPDRFSG | 2622 | QSYDS | 2822 | FGTGTK | 2949 |
| | | PPSVSG | | TGTNSNI | | GTAPKL | | S | | SKSDTSAS | | SLIGS | | LTVL | |
| | | ALGQRV | | | | LIY | | | | LAITGLQA | | I | | | |
| | | TISC | | | | | | | | EDEADYYC | | | | | |
| ADI-75795 | IGLV2-8 | QPVLTQ | 1970 | TGTSSDV | 2172 | WYQQYP | 2329 | EVNKRP | 2394 | GVPDRFSG | 2623 | SSYAG | 2823 | FGGGTK | 2939 |
| | | PPSASG | | GAHNYVS | | DKAPKL | | S | | SKSGDTAS | | SNNWV | | LTVL | |
| | | SPGQSV | | | | MIY | | | | LTVSGLQA | | | | | |
| | | TISC | | | | | | | | EDEADYYC | | | | | |
| ADI-75816 | IGKV3-20 | EIALTQ | 1971 | RASQSVS | 2173 | WYQQKP | 2258 | GASSRA | 2464 | GIPDRFSG | 2624 | QHYGS | 2824 | FGQGTK | 2937 |
| | | SPGTLS | | SNYLA | | GQAPRL | | T | | GGSGTDFT | | SPPYT | | VEIK | |
| | | LSPGER | | | | LIY | | | | LTISRLEP | | | | | |
| | | ATLSC | | | | | | | | EDFAVYYC | | | | | |
| ADI-75725 | IGLV1-40 | QSVVTQ | 1972 | TGTNSNI | 2171 | WYQQLP | 2314 | DINNRP | 2470 | GVPDRFSG | 2625 | QSYDS | 2825 | FGTGTK | 2949 |
| | | PPSVSG | | GAGYDVH | | GTAPKL | | S | | SKSDTSAS | | GLTSP | | LTVL | |
| | | APGQRV | | | | LIY | | | | LAITGLQA | | V | | | |
| | | TISC | | | | | | | | EDEADYFC | | | | | |
| ADI-75686 | IGKV1- | EIQLTQ | 1917 | RASQGIS | 2062 | WYQQIP | 2330 | AASILE | 2385 | GVPSRFRG | 2626 | QQYYS | 2703 | FGGGTK | 2936 |
| | NL1 | SPSSLS | | NSLA | | GKAPKL | | S | | SGSGTDFT | | SLGRT | | VEIK | |
| | | ASVGDR | | | | LLY | | | | LTISSLQP | | | | | |
| | | VTITC | | | | | | | | EDFATYYC | | | | | |
| ADI-75717 | IGLV1-40 | QSVLTQ | 1948 | GAGYDVH | 2143 | WYQQLP | 2331 | VNNNRP | 2471 | GVPDRFSG | 2622 | QSYDS | 2826 | FGGGTK | 2939 |
| | | PPSVSG | | TGSNSNI | | GRAPKL | | S | | SKSDTSAS | | SLSAS | | LTVL | |
| | | APGQRV | | | | LIY | | | | LAITGLQA | | V | | | |
| | | TISC | | | | | | | | EDEADYYC | | | | | |
| ADI-75771 | IGLV2-14 | QPVLTQ | 1973 | TGTSSDV | 2061 | WYQQHP | 2332 | EVSNRP | 2427 | GVSNRFSG | 2627 | SSYTS | 2827 | FGGGTK | 2939 |
| | | PASVSG | | GGYNYVS | | GKVPKL | | S | | SKSGSTAS | | SDSWV | | LTVL | |
| | | SPGQSI | | | | MIY | | | | LTISGLQS | | | | | |
| | | TIPC | | | | | | | | EDEADYYC | | | | | |
| ADI-75651 | IGLV3-21 | SYELTQ | 1919 | GGSNIGS | 2174 | WYQQKP | 2283 | DDSDRP | 2399 | GIPERFSG | 2628 | QVWDS | 2828 | FGGGTK | 2939 |
| | | PPSVSV | | KSVH | | GQAPVL | | S | | SNSGDTAT | | SSDHW | | LTVL | |
| | | APGQTA | | | | VVY | | | | LTISRVEA | | V | | | |
| | | RITC | | | | | | | | GDEADYYC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75638 | GACATCCAGATGACCCAGTCTCCATCCTCCCTGTCTGCGTCTGTAGGAGACAGAGTCGCCA | 3102 | DIQMTQSPSSLSASVGDRVAITCQASQD | 3384 | | |
| | TCACTTGCCAGGCGAGTCAGGACATTAATAAGTATTTAGCTTGGTATCAGCAGAAAGCAGG | | INKYLAWYQQKAGRAPKLLIYDASILET | | | |
| | GAGAGCCCCTAAGCTCCTGATCTACGATGCCTCCATTTTGGAAACAGGGGTCCCATCAAGG | | GVPSRFSGSGSGTHFTLTINSLQTEDIA | | | |
| | TTCAGTGGAAGTGGATCTGGGACACATTTTACTTTGACCATCAACAGCCTGCAGACTGAAG | | TYYCQQFDNVPYTFGQGTKVDIK | | | |
| | ATATTGCAACATATTACTGTCAACAGTTTGATAATGTCCCGTACACTTTTGGCCAGGGGAC | | | | | |
| | CAAGGTGGATATCAAA | | | | | |
| ADI-75800 | GACATCCAGGTGACCCAGTCTCCAGCCACCCTGTCTTTGTCTCCAGGGGAAAGAGCCACCC | 3103 | DIQVTQSPATLSLSPGERATLSCRASQS | 3385 | | |
| | TCTCCTGCAGGGCCAGTCAGAGTGTTAGCAGTTACTTAGCCTGGTACCAACACAAACCTGG | | VSSYLAWYQHKPGQAPRLLIYDASNRAT | | | |
| | CCAGGCTCCCAGGCTCCTCATCTATGATGCATCCAACAGGGCCACTGGCATCCCAGCCAGG | | GIPARFSGSGSGTDFTLTISSLEPEDFA | | | |
| | TTCAGTGGCAGTGGGTCTGGGACAGACTTCACTCTCACCATCAGCAGCCTAGAGCCTGAAG | | VYYCQLRSNWPPFTFGPGTKVEIK | | | |
| | ATTTTGCAGTTTATTACTGTCAGCTACGTAGCAACTGGCCTCCATTCACTTTCGGCCCTGG | | | | | |
| | GACCAAGGTGGAAATCAAA | | | | | |
| ADI-75675 | GATATTGTGATGACCCAGTCTCCGTCCTCCCTGTCTGCTTCTGTGGGAGACAGAGTCTCCA | 3104 | DIVMTQSPSSLSASVGDRVSITCRASQT | 3386 | | |
| | TCACTTGCCGGGCAAGTCAGACCGTAAGAAGTTTTTTAAATTGGTATCAGCAGAAACCAGG | | VRSFLNWYQQKPGKAPKLLVFTAPTVEG | | | |
| | GAAAGCCCCTAAACTCCTGGTCTTTACTGCACCCACTGTGGAAGGTGGAGTCCCCCCGAGG | | GVPPRFSATGSGTDFTLSISGLQPEDFA | | | |
| | TTCAGTGCCACTGGATCTGGCACAGATTTCACTCTCTCCATCAGCGGTCTACAACCTGAGG | | TYFCQQSYSIPYTFGQGTKLEIK | | | |
| | ATTTTGCAACTTACTTCTGTCAACAGAGTTACAGTATCCCGTACACTTTTGGCCAGGGGAC | | | | | |
| | CAAGCTGGAGATCAAA | | | | | |
| ADI-75818 | GACATCCAGATGACCCAGTCTCCATCCTCCCTGTCTGCATCTGTAGGAGACAGAGTAACCA | 3105 | DIQMTQSPSSLSASVGDRVTITCQASQD | 3387 | | |
| | TCACTTGCCAGGCGAGTCAGGACATTAGCGACTATCTAAATTGGTATCAGCAGAAACCAGG | | ISDYLNWYQQKPGKAPKLLIYDALNLKS | | | |
| | GAAAGCCCCTAAACTCCTGATCTACGATGCATTGAATTTGAAATCAGGGGTCCCCTCAAGG | | GVPSRFSGSGSGTDFTFTISSLQPEDIA | | | |
| | TTCAGTGGAAGTGGATCTGGGACAGATTTTACTTTCACCATCAGCAGCCTGCAGCCTGAAG | | TYYCQQYNNFPYTFGQGTKVEIK | | | |
| | ATATTGCAACATATTACTGTCAACAGTACAATAATTTCCCGTACACTTTTGGCCAGGGGAC | | | | | |
| | CAAGGTGGAAATCAAA | | | | | |
| ADI-75741 | CAGTCTGTGGTGACGCAGCCGCCCTCAGTGTCTGGGGCCCTAGGGCAGAGGGTCACCATCT | 3106 | QSVVTQPPSVSGALGQRVTISCTGTNSN | 3388 | | |
| | CCTGCACTGGGACCAACTCCAACATCGGGGCAGGTTATGATGTACACTGGTACCAGCAGCT | | IGAGYDVHWYQQLPGTAPKLLIYDNSNR | | | |
| | TCCAGGAACAGCCCCCAAACTCCTCATCTATGATAATAGTAATCGGCCCTCAGGGGTCCCT | | PSGVPDRFSGSKSDTSASLAITGLQAED | | | |
| | GACCGATTCTCTGGCTCCAAGTCTGACACCTCAGCCTCCCTGGCCATCACTGGGCTCCAGG | | EADYYCQSYDSSLIGSIFGTGTKLTVL | | | |
| | CTGAGGATGAGGCTGATTATTACTGCCAGTCCTATGACAGCAGCCTGATTGGTTCTATCTT | | | | | |
| | CGGGACTGGGACCAAGCTCACCGTCCTA | | | | | |
| ADI-75795 | CAGCCTGTGCTGACTCAGCCCCCCTCCGCGTCCGGGTCTCCTGGACAGTCAGTCACCATCT | 3107 | QPVLTQPPSASGSPGQSVTISCTGTSSD | 3389 | | |
| | CCTGCACTGGAACCAGCAGTGACGTTGGTGCTCATAACTATGTCTCCTGGTACCAACAGTA | | VGAHNYVSWYQQYPDKAPKLMIYEVNKR | | | |
| | CCCAGACAAAGCCCCCAAACTCATGATTTATGAGGTCAATAAGCGGCCCTCAGGGGTCCCT | | PSGVPDRFSGSKSGDTASLTVSGLQAED | | | |
| | GATCGCTTCTCTGGCTCCAAGTCTGGCGACACGGCCTCCCTGACCGTCTCTGGACTCCAGG | | EADYYCSSYAGSNNWVFGGGTKLTVL | | | |
| | CTGAGGATGAGGCTGATTATTATTGCAGCTCATATGCAGGCAGCAACAATTGGGTGTTCGG | | | | | |
| | CGGAGGGACCAAGCTCACCGTCCTA | | | | | |
| ADI-75816 | GAAATTGCACTGACGCAGTCTCCAGGCACCCTGTCTTTGTCTCCAGGGGAAAGAGCCACCC | 3108 | EIALTQSPGTLSLSPGERATLSCRASQS | 3390 | | |
| | TCTCCTGCAGGGCCAGTCAGAGTGTTAGCAGCAACTACTTAGCCTGGTACCAGCAGAAACC | | VSSNYLAWYQQKPGQAPRLLIYGASSRA | | | |
| | TGGCCAGGCTCCCAGGCTCCTCATCTATGGTGCATCCAGCAGGGCCACTGGCATCCCAGAC | | TGIPDRFSGGGSGTDFTLTISRLEPEDF | | | |
| | AGGTTCAGTGGCGGTGGGTCTGGGACAGACTTCACTCTCACCATCAGCAGACTGGAGCCTG | | AVYYCQHYGSSPPYTFGQGTKVEIK | | | |
| | AAGATTTTGCAGTGTATTACTGTCAGCATTATGGGAGCTCACCTCCGTACACTTTTGGCCA | | | | | |
| | GGGGACCAAGGTGGAAATCAAA | | | | | |
| ADI-75725 | CAGTCTGTCGTGACGCAGCCGCCCTCAGTGTCTGGGGCCCCAGGGCAGAGGGTCACCATCT | 3109 | QSVVTQPPSVSGAPGQRVTISCTGTNSN | 3391 | | |
| | CCTGCACTGGGACCAACTCCAACATCGGGGCAGGTTATGATGTCCACTGGTACCAGCAACT | | IGAGYDVHWYQQLPGTAPKLLIYDINNR | | | |
| | TCCAGGAACAGCCCCCAAACTCCTCATCTATGATATCAACAATCGGCCCTCAGGGGTCCCT | | PSGVPDRFSGSKSDTSASLAITGLQAED | | | |
| | GACCGATTCTCTGGCTCCAAGTCTGACACCTCAGCCTCCCTGGCCATCACTGGGCTCCAGG | | EADYFCQSYDSGLTSPVFGTGTKLTVL | | | |
| | CTGAGGATGAGGCTGATTATTTCTGCCAGTCCTATGACAGCGGCCTGACTAGTCCTGTCTT | | | | | |
| | CGGAACTGGGACCAAGCTCACCGTCCTA | | | | | |
| ADI-75686 | GAAATCCAGTTGACCCAGTCTCCATCCTCCCTGTCTGCATCTGTCGGAGACAGAGTCACCA | 3110 | EIQLTQSPSSLSASVGDRVTITCRASQG | 3392 | | |
| | TCACTTGCCGGGCGAGTCAGGGCATTAGCAATTCTTTAGCCTGGTATCAACAGATACCAGG | | ISNSLAWYQQIPGKAPKLLLYAASILES | | | |
| | TAAAGCCCCTAAGCTCCTACTCTATGCTGCATCCATATTGGAAAGTGGGGTCCCATCCAGG | | GVPSRFRGSGSGTDFTLTISSLQPEDFA | | | |
| | TTCAGGGGCAGTGGCTCTGGGACGGATTTCACTCTCACCATCAGCAGCCTGCAGCCTGAAG | | TYYCQQYYSSLGRTFGGGTKVEIK | | | |
| | ATTTTGCAACTTATTACTGTCAACAGTATTATAGTTCCCTCGGGCGCACTTTCGGCGGAGG | | | | | |
| | GACCAAGGTGGAAATCAAA | | | | | |
| ADI-75717 | CAGTCTGTCTTGACGCAGCCGCCCTCAGTGTCTGGGGCCCCAGGGCAGAGGGTCACCATCT | 3111 | QSVLTQPPSVSGAPGQRVTISCTGSNSN | 3393 | | |
| | CCTGCACTGGGAGCAACTCCAACATCGGGGCAGGTTATGATGTGCACTGGTACCAGCAGCT | | IGAGYDVHWYQQLPGRAPKLLIYVNNNR | | | |
| | TCCAGGAAGAGCCCCCAAACTCCTCATCTATGTTAACAACAATCGACCCTCAGGGGTCCCT | | PSGVPDRFSGSKSDTSASLAITGLQAED | | | |
| | GACCGATTCTCTGGCTCCAAGTCTGACACCTCAGCCTCCCTGGCCATCACTGGGCTCCAGG | | EADYYCQSYDSSLSASVFGGGTKLTVL | | | |
| | CTGAGGATGAGGCTGACTATTACTGCCAGTCCTATGACAGCAGCCTGAGTGCCTCGGTTTT | | | | | |
| | CGGCGGAGGGACCAAGCTCACCGTCCTA | | | | | |
| ADI-75771 | CAGCCTGTGCTGACTCAGCCTGCCTCCGTGTCTGGGTCTCCTGGACAGTCGATCACCATCC | 3112 | QPVLTQPASVSGSPGQSITIPCTGTSSD | 3394 | | |
| | CCTGCACTGGAACCAGCAGTGACGTTGGTGGTTATAATTATGTCTCCTGGTACCAACAGCA | | VGGYNYVSWYQQHPGKVPKLMIYEVSNR | | | |
| | CCCAGGCAAAGTCCCCAAACTCATGATTTATGAGGTCAGTAATCGGCCCTCAGGGGTTTCT | | PSGVSNRFSGSKSGSTASLTISGLQSED | | | |
| | AATCGCTTCTCTGGCTCCAAGTCTGGCAGCACGGCCTCCCTGACCATCTCTGGGCTCCAGT | | EADYYCSSYTSSDSWVFGGGTKLTVL | | | |
| | CTGAGGACGAGGCTGATTATTATTGCAGCTCATATACAAGTAGCGACTCTTGGGTGTTCGG | | | | | |
| | CGGAGGGACCAAGCTCACCGTCCTA | | | | | |
| ADI-75651 | TCGTATGAGCTGACTCAGCCACCCTCGGTGTCAGTGGCCCCAGGACAGACGGCCAGGATTA | 3113 | SYELTQPPSVSVAPGQTARITCGGSNIG | 3395 | | |
| | CCTGTGGGGGAAGCAACATTGGAAGTAAAAGTGTGCACTGGTACCAGCAGAAGCCAGGCCA | | SKSVHWYQQKPGQAPVLVVYDDSDRPSG | | | |
| | GGCCCCTGTGCTGGTCGTCTATGATGATAGCGACCGGCCCTCAGGGATCCCTGAGCGATTC | | IPERFSGSNSGDTATLTISRVEAGDEAD | | | |
| | TCTGGCTCCAACTCTGGGGACACGGCCACCCTGACCATCAGCAGGGTCGAAGCCGGGGATG | | YYCQVWDSSSDHWVFGGGTKLTVL | | | |
| | AGGCCGACTATTACTGTCAGGTGTGGGATAGTAGTAGTGATCATTGGGTGTTCGGCGGAGG | | | | | |
| | GACCAAGCTCACCGTCCTA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75813 | IGKV1D- | DIRMTQ | 1974 | RASQSIT | 2175 | WYQQKP | 2257 | AASSLQ | 2391 | GVPSRFSG | 2629 | LQTYS | 2829 | FGQGTK | 2937 |
| | 39 | SPSSLS | | SYLH | | GKAPKL | | S | | SGSGTDFT | | THSWT | | VEIK | |
| | | ASVGDS | | | | LIY | | | | LTITSLKP | | | | | |
| | | VTITC | | | | | | | | EDFATYYC | | | | | |
| ADI-75750 | IGKV1D- | DILMTQ | 1975 | RASQSIN | 2176 | WYQQKP | 2333 | AASSLQ | 2391 | GVPSRFGG | 2630 | QQSYS | 2830 | FGQGTK | 2937 |
| | 39 | SPSSLS | | HYLN | | GKAPKL | | S | | SGSETDFT | | TPHGS | | VEIK | |
| | | ASVGDR | | | | LIS | | | | LTISSLEP | | | | | |
| | | VTITC | | | | | | | | EDFATYYC | | | | | |
| ADI-75608 | IGKV1D- | EIVLTQ | 1976 | RASQSVG | 2177 | WYQQKP | 2257 | AASNLQ | 2408 | GVPSRFSG | 2606 | QQSYT | 2749 | FGQGTK | 2937 |
| | 39 | SPSSLS | | RYLN | | GKAPKL | | S | | SGSGTDFT | | THMYT | | VEIK | |
| | | ATVGDR | | | | LIY | | | | LTISSLQP | | | | | |
| | | VTITC | | | | | | | | EDFASYYC | | | | | |
| ADI-75663 | IGKV1- | DIQMTQ | 1878 | RASQGIS | 2062 | WYQQLP | 2334 | AASILE | 2385 | GVPSRFRG | 2626 | QQYYS | 2703 | FGGGTK | 2936 |
| | NL1 | SPSSLS | | NSLA | | GKAPKL | | S | | SGSGTDFT | | SLGRT | | VEIK | |
| | | ASVGDR | | | | LLY | | | | LTISSLQP | | | | | |
| | | VTITC | | | | | | | | EDFATYYC | | | | | |
| ADI-75652 | IGKV1-33 | DIQMTQ | 1977 | QASQDIN | 2160 | WYQQKP | 2257 | DASNLE | 2416 | GVPSRFSG | 2631 | HQYDN | 2831 | FGQGTK | 2937 |
| | | SPSSLS | | KYLN | | GKAPKL | | T | | SGSGRHFT | | LPRT | | VEIK | |
| | | ASVGDS | | | | LIY | | | | FTISSLQP | | | | | |
| | | VTITC | | | | | | | | EDIGTYYC | | | | | |
| ADI-75567 | IGKV4-1 | DIPMTQ | 1978 | RSSQSVL | 2178 | WYQQKP | 2290 | WASTRE | 2422 | GVPDRFSG | 2632 | QQYYS | 2832 | FGPGTK | 2942 |
| | | SPDSLA | | YSSNDQN | | GQPPKL | | S | | SGSGTHFT | | SPPT | | VEIK | |
| | | VSLGER | | YLA | | LIY | | | | LTISSLQA | | | | | |
| | | ATINC | | | | | | | | EDVAVYYC | | | | | |
| ADI-75670 | IGKV1D- | EIQMTQ | 1979 | RASQSIS | 2050 | WYQQKP | 2257 | HALSLQ | 2472 | GVPSRFSG | 2534 | QQSYT | 2833 | FGQGTK | 2944 |
| | 39 | SPSSLS | | SYLN | | GKAPKL | | S | | SGSGTDFT | | TPYT | | VDIK | |
| | | AYVGDR | | | | LIY | | | | LTISSLQP | | | | | |
| | | VTITC | | | | | | | | EDFATYYC | | | | | |
| ADI-75602 | IGKV4-1 | DIQLTQ | 1980 | RSSQSVL | 2178 | WYQQKP | 2290 | WASTRE | 2422 | GVPDRFSG | 2632 | QQYYS | 2832 | FGPGTK | 2950 |
| | | TPDSLA | | YSSNDQN | | GQPPKL | | S | | SGSGTHFT | | SPPT | | VDIK | |
| | | VSLGER | | YLA | | LIY | | | | LTISSLQA | | | | | |
| | | ATINC | | | | | | | | EDVAVYYC | | | | | |
| ADI-75774 | IGKV1-9 | EIEMTQ | 1981 | RASQGIS | 2140 | WYQQKP | 2257 | GASTLQ | 2447 | GVPLRFSG | 2595 | QHLDS | 2792 | FGQGTK | 2937 |
| | | SPSFLS | | SFLA | | GKAPKL | | S | | SGSGTEFT | | YPRYT | | VEIK | |
| | | ASVGDR | | | | LIY | | | | LTISSLQP | | | | | |
| | | VTITC | | | | | | | | EDFATYYC | | | | | |
| ADI-75736 | IGKV3-11 | EIVLTQ | 1945 | RASQSVS | 2168 | WYQQKP | 2258 | DASNRA | 2448 | GIPARFSG | 2607 | QQRSN | 2834 | FGGGTK | 2938 |
| | | SPATLS | | SYLA | | GQAPRL | | T | | SGSGTDFT | | WPPGL | | VDIK | |
| | | LSPGER | | | | LIY | | | | LTISSLEP | | T | | | |
| | | ATLSC | | | | | | | | EDFAVYYC | | | | | |
| ADI-75586 | IGLV2-14 | QPVLTQ | 1933 | TGTSSDI | 2179 | WYQQHP | 2335 | DVTNRP | 2473 | GVSYRFSG | 2633 | SSYTS | 2835 | FGTGTK | 2949 |
| | | PASVSG | | GDYNYVS | | GKAPKL | | S | | FKSGNTAS | | SDTYV | | LTVL | |
| | | SPGQSI | | | | MVY | | | | LTISGLQA | | | | | |
| | | TISC | | | | | | | | EDEADYYC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75813 | GACATCCGGATGACCCAGTCTCCATCCTCCCTGTCTGCATCTGTAGGAGACAGCGTCACCA | 3114 | DIRMTQSPSSLSASVGDSVTITCRASQS | 3396 | | |
| | TCACTTGCCGGGCAAGTCAGAGCATTACCAGCTATTTACATTGGTATCAGCAGAAACCAGG | | ITSYLHWYQQKPGKAPKLLIYAASSLQS | | | |
| | GAAAGCCCCTAAGCTCCTGATCTATGCTGCATCCAGTTTGCAAAGTGGGGTCCCATCAAGG | | GVPSRFSGSGSGTDFTLTITSLKPEDFA | | | |
| | TTCAGTGGCAGTGGATCTGGGACAGATTTCACTCTCACCATCACCAGTCTGAAACCTGAAG | | TYYCLQTYSTHSWTFGQGTKVEIK | | | |
| | ATTTTGCAACTTACTACTGTCTACAGACTTACAGTACCCATTCGTGGACGTTCGGCCAAGG | | | | | |
| | GACCAAGGTGGAAATCAAA | | | | | |
| ADI-75750 | GACATCCTGATGACCCAGTCTCCATCCTCCCTGTCTGCATCTGTTGGAGACAGAGTCACCA | 3115 | DILMTQSPSSLSASVGDRVTITCRASQS | 3397 | | |
| | TCACTTGCCGGGCAAGTCAGAGCATTAACCACTATTTAAATTGGTATCAGCAGAAGCCAGG | | INHYLNWYQQKPGKAPKLLISAASSLQS | | | |
| | GAAGGCCCCCAAGCTCCTGATCTCTGCTGCATCCAGTTTGCAAAGTGGGGTCCCATCAAGG | | GVPSRFGGSGSETDFTLTISSLEPEDFA | | | |
| | TTCGGTGGCAGTGGATCTGAGACAGATTTCACTCTCACCATCAGCAGTCTGGAACCTGAAG | | TYYCQQSYSTPHGSFGQGTKVEIK | | | |
| | ATTTTGCAACTTACTACTGTCAACAGAGTTACAGTACCCCCCATGGGAGTTTTGGCCAGGG | | | | | |
| | GACCAAGGTGGAAATCAAA | | | | | |
| ADI-75608 | GAAATCGTGTTGACCCAGTCTCCATCCTCGCTGTCTGCAACTGTAGGAGACAGAGTCACCA | 3116 | EIVLTQSPSSLSATVGDRVTITCRASQS | 3398 | | |
| | TCACTTGCCGGGCAAGTCAGAGCGTTGGCAGGTATTTAAATTGGTATCAGCAGAAACCAGG | | VGRYLNWYQQKPGKAPKLLIYAASNLQS | | | |
| | GAAAGCCCCTAAACTCCTGATCTATGCTGCATCCAATTTGCAAAGTGGGGTCCCGTCAAGG | | GVPSRFSGSGSGTDFTLTISSLQPEDFA | | | |
| | TTCAGTGGCAGTGGATCTGGGACAGATTTCACTCTCACCATCAGCAGTCTGCAACCTGAAG | | SYYCQQSYTTHMYTFGQGTKVEIK | | | |
| | ATTTTGCAAGTTACTACTGTCAACAGAGTTACACGACCCACATGTACACTTTTGGCCAGGG | | | | | |
| | GACCAAGGTGGAGATCAAA | | DIQMTQSPSSLSASVGDRVTITCRASQG | | | |
| ADI-75663 | GACATCCAGATGACCCAGTCTCCATCCTCCCTGTCTGCATCTGTCGGAGACAGAGTCACCA | 3117 | ISNSLAWYQQLPGKAPKLLLYAASILES | 3399 | | |
| | TCACTTGCCGGGCGAGTCAGGGCATTAGCAATTCTTTAGCCTGGTATCAACAACTACCAGG | | GVPSRFRGSGSGTDFTLTISSLQPEDFA | | | |
| | GAAAGCCCCTAAACTCCTGCTCTATGCTGCATCCATATTGGAAAGTGGGGTCCCATCCAGG | | TYYCQQYYSSLGRTFGGGTKVEIK | | | |
| | TTCAGGGGCAGTGGTTCTGGGACGGATTTCACTCTCACCATCAGCAGCCTGCAGCCTGAAG | | | | | |
| | ATTTTGCAACTTATTACTGTCAACAGTATTATAGTTCCCTCGGGCGCACTTTCGGCGGAGG | | | | | |
| | GACCAAGGTGGAGATCAAA | | | | | |
| ADI-75652 | GACATCCAGATGACCCAGTCTCCATCCTCCCTGTCTGCATCTGTAGGAGACAGCGTCACCA | 3118 | DIQMTQSPSSLSASVGDSVTITCQASQD | 3400 | | |
| | TCACTTGCCAGGCGAGTCAGGACATTAACAAGTATTTAAATTGGTATCAGCAGAAACCAGG | | INKYLNWYQQKPGKAPKLLIYDASNLET | | | |
| | GAAAGCCCCTAAGCTCCTGATCTACGATGCATCCAATTTGGAAACAGGGGTCCCATCAAGG | | GVPSRFSGSGSGRHFTFTISSLQPEDIG | | | |
| | TTCAGTGGAAGTGGATCTGGGAGACATTTTACTTTCACCATCAGCAGCCTGCAGCCTGAAG | | TYYCHQYDNLPRTFGQGTKVEIK | | | |
| | ATATTGGAACATATTACTGTCACCAGTATGATAATCTCCCTAGGACGTTCGGCCAAGGGAC | | | | | |
| | CAAGGTGGAAATCAAA | | | | | |
| ADI-75567 | GACATCCCGATGACCCAGTCTCCAGATTCCCTGGCTGTGTCTCTGGGCGAGAGGGCCACCA | 3119 | DIPMTQSPDSLAVSLGERATINCRSSQS | 3401 | | |
| | TCAACTGCAGGTCCAGCCAGAGTGTTTTGTACAGCTCCAACGATCAGAACTACTTAGCTTG | | VLYSSNDQNYLAWYQQKPGQPPKLLIYW | | | |
| | GTACCAGCAGAAACCAGGACAGCCTCCTAAGTTGCTCATTTACTGGGCATCTACCCGGGAA | | ASTRESGVPDRFSGSGSGTHFTLTISSL | | | |
| | TCCGGGGTCCCTGACCGATTCAGTGGCAGCGGGTCTGGGACACATTTCACTCTCACCATCA | | QAEDVAVYYCQQYYSSPPTFGPGTKVEI | | | |
| | GCAGCCTGCAGGCTGAAGATGTGGCAGTTTATTACTGTCAGCAATATTATAGTAGTCCTCC | | K | | | |
| | CACTTTCGGCCCTGGGACCAAGGTGGAAATCAAA | | | | | |
| ADI-75670 | GAAATCCAGATGACCCAGTCTCCATCCTCCCTGTCTGCATATGTAGGAGACAGAGTCACCA | 3120 | EIQMTQSPSSLSAYVGDRVTITCRASQS | 3402 | | |
| | TCACTTGCCGGGCAAGTCAGAGCATTAGCAGCTATTTGAATTGGTATCAGCAGAAACCAGG | | ISSYLNWYQQKPGKAPKLLIYHALSLQS | | | |
| | GAAAGCCCCTAAACTCCTGATCTATCATGCACTCAGTTTGCAAAGTGGGGTCCCATCAAGG | | GVPSRFSGSGSGTDFTLTISSLQPEDFA | | | |
| | TTCAGTGGCAGTGGATCTGGGACAGATTTCACTCTCACCATCAGCAGTCTGCAACCTGAAG | | TYYCQQSYTTPYTFGQGTKVDIK | | | |
| | ATTTTGCAACTTACTACTGTCAACAGAGTTACACTACCCCGTACACTTTTGGCCAGGGGAC | | | | | |
| | CAAGGTGGATATCAAA | | | | | |
| ADI-75602 | GACATCCAGTTGACCCAGACTCCAGATTCCCTGGCTGTGTCTCTGGGCGAGAGGGCCACCA | 3121 | DIQLTQTPDSLAVSLGERATINCRSSQS | 3403 | | |
| | TCAACTGCAGGTCCAGCCAGAGTGTTTTGTACAGCTCCAACGATCAGAACTACTTAGCTTG | | VLYSSNDQNYLAWYQQKPGQPPKLLIYW | | | |
| | GTACCAGCAGAAACCAGGACAGCCTCCTAAGTTGCTCATTTACTGGGCATCTACCCGGGAA | | ASTRESGVPDRFSGSGSGTHFTLTISSL | | | |
| | TCCGGGGTCCCTGACCGATTCAGTGGCAGCGGGTCTGGGACACATTTCACTCTCACCATCA | | QAEDVAVYYCQQYYSSPPTFGPGTKVDI | | | |
| | GCAGCCTGCAGGCTGAAGATGTGGCAGTTTATTACTGTCAGCAATATTATAGTAGTCCTCC | | K | | | |
| | CACTTTCGGCCCTGGGACCAAAGTGGATATCAAA | | | | | |
| ADI-75774 | GAAATCGAGATGACCCAGTCTCCATCCTTCCTGTCTGCATCTGTAGGAGACAGAGTCACCA | 3122 | EIEMTQSPSFLSASVGDRVTITCRASQG | 3404 | | |
| | TCACTTGCCGGGCCAGTCAGGGCATTAGCAGTTTTTTAGCCTGGTATCAGCAAAAACCAGG | | ISSFLAWYQQKPGKAPKLLIYGASTLQS | | | |
| | GAAAGCCCCTAAGCTCCTGATCTATGGTGCATCCACTTTGCAAAGTGGGGTCCCATTAAGG | | GVPLRFSGSGSGTEFTLTISSLQPEDFA | | | |
| | TTCAGCGGCAGTGGATCTGGGACAGAATTCACTCTCACAATCAGCAGCCTGCAGCCTGAAG | | TYYCQHLDSYPRYTFGQGTKVEIK | | | |
| | ATTTTGCAACTTATTACTGTCAACACCTTGATAGTTACCCTCGGTACACTTTTGGCCAGGG | | | | | |
| | GACCAAGGTGGAAATCAAA | | | | | |
| ADI-75736 | GAAATTGTATTGACGCAGTCTCCAGCCACCCTGTCTTTGTCTCCAGGGGAAAGAGCCACCC | 3123 | EIVLTQSPATLSLSPGERATLSCRASQS | 3405 | | |
| | TCTCCTGCAGGGCCAGTCAGAGTGTTAGCAGCTACTTAGCCTGGTACCAACAGAAACCTGG | | VSSYLAWYQQKPGQAPRLLIYDASNRAT | | | |
| | CCAGGCTCCCAGGCTCCTCATCTATGATGCATCCAACAGGGCCACTGGCATCCCAGCCAGG | | GIPARFSGSGSGTDFTLTISSLEPEDFA | | | |
| | TTCAGTGGCAGTGGGTCTGGGACAGACTTCACTCTCACCATCAGCAGCCTAGAGCCTGAAG | | VYYCQQRSNWPPGLTFGGGTKVDIK | | | |
| | ATTTTGCAGTTTATTACTGTCAGCAGCGTAGCAACTGGCCTCCGGGGCTCACTTTCGGCGG | | | | | |
| | AGGGACCAAAGTGGATATCAAA | | | | | |
| ADI-75586 | CAGCCTGTGCTGACTCAGCCTGCCTCCGTGTCTGGGTCTCCTGGACAGTCGATCACCATCT | 3124 | QPVLTQPASVSGSPGQSITISCTGTSSD | 3406 | | |
| | CCTGCACTGGAACCAGCAGTGACATTGGTGATTATAACTATGTCTCCTGGTACCAACAACA | | IGDYNYVSWYQQHPGKAPKLMVYDVTNR | | | |
| | CCCAGGCAAAGCCCCCAAACTCATGGTCTATGATGTCACTAATCGGCCCTCAGGGGTTTCT | | PSGVSYRFSGFKSGNTASLTISGLQAED | | | |
| | TATCGCTTCTCTGGCTTCAAGTCTGGCAACACGGCCTCCCTGACCATCTCTGGACTCCAGG | | EADYYCSSYTSSDTYVFGTGTKLTVL | | | |
| | CTGAGGACGAGGCTGATTATTACTGCAGCTCATATACAAGCAGCGACACATATGTCTTCGG | | | | | |
| | AACTGGGACCAAGCTCACCGTCCTA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75687 | IGKV1- | DIQMTQ | 1878 | RASQGIS | 2062 | WYQHIP | 2336 | AASILE | 2385 | GVPSRFRG | 2634 | QQYYS | 2703 | FGGGTK | 2936 |
| | NL1 | SPSSLS | | NSLA | | GKAPKL | | S | | SGSGTDFT | | SLGRT | | VEIK | |
| | | ASVGDR | | | | LLY | | | | LTISSLQP | | | | | |
| | | VTITC | | | | | | | | EDFASYYC | | | | | |
| ADI-75694 | IGLV3-21 | QPVLTQ | 1982 | GGDNIGS | 2180 | WYQQKP | 2283 | DDSDRP | 2399 | GIPERFFG | 2635 | QVWDS | 2836 | FGTGTK | 2949 |
| | | PPSVSV | | KGVH | | GQAPVL | | S | | SNSGNTAT | | TSDHP | | LTVL | |
| | | APGQTA | | | | VVY | | | | LTISRVEA | | AYV | | | |
| | | RITC | | | | | | | | GDEADYFC | | | | | |
| ADI-75711 | IGLV3-21 | QSELTQ | 1983 | GGNNIGS | 2181 | WYQQRP | 2277 | DDSGRP | 2474 | GIPERFSG | 2636 | QVWDS | 2837 | FGGGTK | 2939 |
| | | PPSVSV | | NSVH | | GQAPVL | | S | | SKSGNTAT | | SSDPV | | LTVL | |
| | | APRQTA | | | | VVY | | | | LTISRVEA | | V | | | |
| | | RITC | | | | | | | | GDEADYYC | | | | | |
| ADI-75704 | IGLV2-14 | QPVLTQ | 1933 | TGTSSDV | 2182 | WYQLHP | 2337 | EVNNRP | 2475 | GISTRFSG | 2637 | SSYTS | 2838 | FGTGTK | 2945 |
| | | PASVSG | | GSYDYVS | | GKAPKL | | S | | SKSGNTAS | | NIV | | VTVL | |
| | | SPGQSI | | | | MIY | | | | LTISGLQA | | | | | |
| | | TISC | | | | | | | | EDEADYYC | | | | | |
| ADI-75631 | IGKV2D- | DIVMTQ | 1984 | RSNQSLL | 2183 | WYLHKP | 2338 | LGSNRA | 2387 | GVPDRFSG | 2638 | VQALQ | 2839 | FGQGTK | 2937 |
| | 28 | SPLSLP | | HSHGYIY | | GQSPQL | | S | | SGSGTDFT | | TPFMY | | VEIK | |
| | | VTPGEP | | LD | | LIY | | | | LSISRVEP | | T | | | |
| | | ASISC | | | | | | | | EDVGVYYC | | | | | |
| ADI-75773 | IGLV1-40 | QSVLTQ | 1948 | TGSNSNI | 2143 | WYQQLP | 2314 | DNNNRP | 2476 | GVPDRFSG | 2622 | QSYDS | 2840 | FGGGTQ | 2948 |
| | | PPSVSG | | GAGYDVH | | GTAPKL | | S | | SKSDTSAS | | SLSDS | | LTVL | |
| | | APGQRV | | | | LIY | | | | LAITGLQA | | V | | | |
| | | TISC | | | | | | | | EDEADYYC | | | | | |
| ADI-75798 | IGKV1-33 | EIQMTQ | 1985 | QASQDIS | 2184 | WYQQKP | 2257 | DASNLE | 2416 | GVPSRFSG | 2639 | QQYDN | 2841 | FGGGTK | 2936 |
| | | TPSSLS | | KFLN | | GKAPKL | | T | | SGSGTGFT | | LPVT | | VEIK | |
| | | ASVGDR | | | | LIY | | | | FTISSLQP | | | | | |
| | | VTITC | | | | | | | | EDIATYYC | | | | | |
| ADI-75782 | IGLV1-40 | QSVLTQ | 1948 | TGSRSNI | 2185 | WYQQLP | 2339 | GNSNRP | 2477 | GVPDRFSG | 2640 | QSYDS | 2842 | FGTGTK | 2949 |
| | | PPSVSG | | GAGYDVH | | GTVPKL | | S | | SKSGSSAS | | SLTFS | | LTVL | |
| | | APGQRV | | | | LIY | | | | LAITGLQA | | AFYV | | | |
| | | ITISC | | | | | | | | EDEAVYYC | | | | | |
| ADI-75858 | IGKV1D- | DIVMTQ | 1986 | RASQSIS | 2134 | WYQQKP | 2272 | TASTLQ | 2478 | GVPSRFSG | 2614 | QQSYT | 2843 | FGQGTK | 2937 |
| | 39 | SPSSLS | | RYLN | | GKAPKL | | T | | SGSGTDFT | | TPPLW | | VEIK | |
| | | ASVGAR | | | | LIH | | | | LTISNLQP | | T | | | |
| | | VTITC | | | | | | | | EDFATYYC | | | | | |
| ADI-75712 | IGLV1-40 | QSVVTQ | 1972 | TGSSSSI | 2186 | WYQQLP | 2314 | ANSNRP | 2479 | GVPDRFSG | 2641 | QSYAS | 2844 | FGGGTK | 2939 |
| | | PPSVSG | | GAGYDVH | | GTAPKL | | S | | SKSGTSAS | | SVSGV | | LTVL | |
| | | APGQRV | | | | LIY | | | | LAITGFQA | | I | | | |
| | | TISC | | | | | | | | EDEDDYYC | | | | | |
| ADI-75716 | IGKV1-9 | DIQMTQ | 1987 | RASQGIS | 2187 | WYQQKP | 2257 | AASTLQ | 2392 | GVPSRFSG | 2642 | QQLNS | 2845 | FGQGTR | 2940 |
| | | SPSFLS | | NYLA | | GKAPKL | | S | | GGSGTEFT | | NPPIT | | LEIK | |
| | | ASVGDR | | | | LIY | | | | LTISSLQP | | | | | |
| | | VTITC | | | | | | | | EDFATYYC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75687 | GACATCCAGATGACCCAGTCTCCATCCTCCCTGTCTGCATCTGTCGGAGACAGAGTCACCA | 3125 | DIQMTQSPSSLSASVGDRVTITCRASQG | 3407 | | |
| | TCACTTGCCGGGCGAGTCAGGGCATTAGCAATTCTTTAGCCTGGTATCAACACATACCAGG | | ISNSLAWYQHIPGKAPKLLLYAASILES | | | |
| | GAAAGCCCCTAAGCTCCTACTCTATGCTGCATCCATATTGGAAAGTGGGGTCCCATCCAGG | | GVPSRFRGSGSGTDFTLTISSLQPEDFA | | | |
| | TTCAGGGGCAGTGGCTCTGGGACGGATTTCACTCTCACCATCAGCAGCCTGCAGCCTGAAG | | SYYCQQYYSSLGRTFGGGTKVEIK | | | |
| | ATTTTGCAAGTTATTACTGTCAACAGTATTACAGTTCCCTCGGGCGCACTTTCGGCGGAGG | | | | | |
| | GACCAAGGTGGAAATCAAA | | | | | |
| ADI-75694 | CAGCCAGTGCTGACTCAGCCCCCCTCGGTGTCAGTGGCCCCGGGACAGACGGCCAGGATTA | 3126 | QPVLTQPPSVSVAPGQTARITCGGDNIG | 3408 | | |
| | CCTGTGGGGGAGACAACATTGGGAGCAAAGGTGTTCACTGGTACCAGCAGAAGCCAGGCCA | | SKGVHWYQQKPGQAPVLVVYDDSDRPSG | | | |
| | GGCCCCTGTGTTGGTCGTTTATGATGATAGCGACCGGCCCTCAGGGATCCCTGAGCGATTC | | IPERFFGSNSGNTATLTISRVEAGDEAD | | | |
| | TTTGGCTCCAACTCGGGGAACACGGCCACCCTGACCATCAGCAGGGTCGAAGCCGGGGATG | | YFCQVWDSTSDHPAYVFGTGTKLTVL | | | |
| | AGGCCGACTACTTCTGTCAGGTGTGGGATAGTACGAGTGATCATCCGGCTTATGTCTTCGG | | | | | |
| | AACTGGGACCAAGCTCACCGTCCTA | | | | | |
| ADI-75711 | CAGTCTGAGCTGACTCAGCCACCCTCGGTGTCAGTGGCCCCACGACAGACGGCCAGGATTA | 3127 | QSELTQPPSVSVAPRQTARITCGGNNIG | 3409 | | |
| | CCTGTGGGGGAAACAACATTGGAAGTAATAGTGTGCATTGGTACCAGCAGAGGCCAGGCCA | | SNSVHWYQQRPGQAPVLVVYDDSGRPSG | | | |
| | GGCCCCTGTGCTGGTCGTCTATGATGATAGCGGCCGGCCCTCAGGGATCCCTGAGCGATTT | | IPERFSGSKSGNTATLTISRVEAGDEAD | | | |
| | TCTGGCTCCAAGTCTGGGAACACGGCCACCCTGACCATCAGCAGGGTCGAAGCCGGGGATG | | YYCQVWDSSSDPVVFGGGTKLTVL | | | |
| | AGGCCGACTATTACTGTCAGGTGTGGGATAGTAGTAGTGATCCTGTGGTTTTCGGCGGAGG | | | | | |
| | GACCAAGCTCACCGTCCTA | | | | | |
| ADI-75704 | CAGCCTGTCCTGACTCAGCCTGCCTCCGTGTCTGGGTCTCCTGGACAGTCGATCACCATCT | 3128 | QPVLTQPASVSGSPGQSITISCTGTSSD | 3410 | | |
| | CCTGCACTGGAACCAGCAGTGACGTTGGTAGTTATGACTATGTCTCCTGGTACCAACTACA | | VGSYDYVSWYQLHPGKAPKLMIYEVNNR | | | |
| | CCCAGGCAAAGCCCCCAAACTCATGATTTATGAGGTCAATAATCGGCCCTCAGGGATTTCT | | PSGISTRFSGSKSGNTASLTISGLQAED | | | |
| | ACTCGCTTCTCTGGCTCCAAGTCTGGCAACACGGCCTCCCTGACCATCTCTGGGCTCCAGG | | EADYYCSSYTSNIVFGTGTKVTVL | | | |
| | CTGAGGACGAGGCTGATTATTACTGCAGCTCATATACAAGCAACATCGTCTTCGGAACTGG | | | | | |
| | GACCAAGGTCACCGTCCTA | | | | | |
| ADI-75631 | GACATTGTGATGACGCAGTCTCCACTCTCCCTGCCCGTCACCCCTGGAGAGCCGGCCTCCA | 3129 | DIVMTQSPLSLPVTPGEPASISCRSNQS | 3411 | | |
| | TCTCCTGCAGGTCTAATCAGAGCCTCCTGCATAGTCATGGATACATCTATTTGGATTGGTA | | LLHSHGYIYLDWYLHKPGQSPQLLIYLG | | | |
| | CCTGCATAAGCCAGGGCAGTCTCCGCAGCTCCTGATCTATTTGGGTTCTAATCGGGCCTCC | | SNRASGVPDRFSGSGSGTDFTLSISRVE | | | |
| | GGGGTCCCTGACAGGTTCAGTGGCAGTGGATCAGGCACAGATTTTACACTGAGTATCAGCA | | PEDVGVYYCVQALQTPFMYTFGQGTKVE | | | |
| | GAGTGGAGCCTGAGGATGTTGGGGTTTATTACTGCGTGCAAGCTCTACAGACTCCCTTCAT | | IK | | | |
| | GTACACTTTTGGCCAGGGGACCAAGGTGGAGATCAAA | | | | | |
| ADI-75773 | CAGTCTGTGTTGACGCAGCCGCCCTCAGTGTCTGGGGCCCCAGGGCAGAGGGTCACCATCT | 3130 | QSVLTQPPSVSGAPGQRVTISCTGSNSN | 3412 | | |
| | CCTGCACTGGGAGCAACTCCAACATCGGGGCAGGTTATGATGTTCACTGGTATCAGCAGCT | | IGAGYDVHWYQQLPGTAPKLLIYDNNNR | | | |
| | TCCAGGAACAGCCCCCAAACTCCTCATCTATGACAACAACAATCGGCCCTCAGGGGTCCCT | | PSGVPDRFSGSKSDTSASLAITGLQAED | | | |
| | GACCGATTCTCTGGCTCCAAGTCTGACACCTCAGCCTCCCTGGCCATCACTGGGCTCCAGG | | EADYYCQSYDSSLSDSVFGGGTQLTVL | | | |
| | CTGAGGATGAGGCTGATTATTACTGCCAGTCCTATGACAGCAGCCTGAGTGATTCGGTGTT | | | | | |
| | CGGCGGAGGCACCCAGCTGACCGTCCTC | | | | | |
| ADI-75798 | GAAATCCAGATGACCCAGACTCCATCCTCCCTGTCTGCATCTGTAGGAGACAGAGTCACCA | 3131 | EIQMTQTPSSLSASVGDRVTITCQASQD | 3413 | | |
| | TCACTTGCCAGGCGAGTCAGGACATTAGCAAGTTTTTAAATTGGTATCAGCAGAAACCAGG | | ISKFLNWYQQKPGKAPKLLIYDASNLET | | | |
| | GAAAGCCCCTAAGCTCCTGATCTACGATGCATCTAATCTGGAAACAGGGGTCCCATCAAGG | | GVPSRFSGSGSGTGFTFTISSLQPEDIA | | | |
| | TTCAGTGGAAGTGGATCTGGGACAGGTTTTACTTTCACCATCAGCAGCCTGCAGCCTGAAG | | TYYCQQYDNLPVTFGGGTKVEIK | | | |
| | ATATTGCAACGTATTACTGTCAACAGTATGATAATCTTCCGGTTACTTTCGGCGGAGGGAC | | | | | |
| | CAAGGTGGAGATCAAA | | | | | |
| ADI-75782 | CAGTCTGTGCTGACGCAGCCGCCCTCAGTGTCTGGGGCCCCAGGGCAGAGGGTCACCATCT | 3132 | QSVLTQPPSVSGAPGQRVTISCTGSRSN | 3414 | | |
| | CCTGCACTGGGAGCAGGTCCAACATCGGGGCAGGTTATGATGTACACTGGTACCAGCAGCT | | IGAGYDVHWYQQLPGTVPKLLIYGNSNR | | | |
| | TCCAGGAACAGTCCCCAAACTCCTCATCTATGGCAACAGCAATCGGCCCTCAGGGGTCCCT | | PSGVPDRFSGSKSGSSASLAITGLQAED | | | |
| | GACCGATTCTCTGGCTCCAAGTCTGGCTCCTCAGCTTCCCTGGCCATCACTGGGCTCCAGG | | EAVYYCQSYDSSLTFSAFYVFGTGTKLT | | | |
| | CTGAGGATGAGGCTGTTTATTACTGCCAGTCCTATGACAGCAGCCTGACTTTTTCGGCTTT | | VL | | | |
| | TTATGTCTTCGGAACTGGGACCAAGCTCACCGTCCTA | | | | | |
| ADI-75858 | GACATCGTGATGACCCAGTCTCCATCCTCCCTGTCTGCCTCTGTAGGAGCCAGAGTCACCA | 3133 | DIVMTQSPSSLSASVGARVTITCRASQS | 3415 | | |
| | TCACTTGCCGGGCAAGTCAGAGCATTAGCAGATATTTAAATTGGTATCAGCAGAAACCAGG | | ISRYLNWYQQKPGKAPKLLIHTASTLQT | | | |
| | GAAAGCCCCTAAGCTCCTGATCCATACTGCATCCACTTTGCAAACTGGGGTCCCATCAAGG | | GVPSRFSGSGSGTDFTLTISNLQPEDFA | | | |
| | TTCAGTGGCAGTGGGTCTGGGACAGATTTCACTCTCACCATCAGCAATCTGCAACCTGAAG | | TYYCQQSYTTPPLWTFGQGTKVEIK | | | |
| | ATTTTGCAACTTACTACTGTCAACAGAGTTACACTACCCCTCCATTGTGGACGTTCGGCCA | | | | | |
| | GGGGACCAAGGTGGAAATCAAA | | | | | |
| ADI-75712 | CAGTCTGTGGTGACGCAGCCGCCCTCAGTGTCTGGGGCCCCAGGGCAGAGGGTCACCATCT | 3134 | QSVVTQPPSVSGAPGQRVTISCTGSSSS | 3416 | | |
| | CCTGCACTGGGAGCAGCTCCAGCATCGGGGCAGGTTATGATGTACACTGGTACCAGCAGCT | | IGAGYDVHWYQQLPGTAPKLLIYANSNR | | | |
| | TCCAGGAACAGCCCCCAAACTCCTCATCTATGCTAACAGCAATCGGCCCTCAGGGGTCCCT | | PSGVPDRFSGSKSGTSASLAITGFQAED | | | |
| | GACCGATTTTCTGGCTCCAAGTCTGGCACCTCAGCCTCCCTGGCCATCACTGGGTTCCAGG | | EDDYYCQSYASSVSGVIFGGGTKLTVL | | | |
| | CTGAGGATGAGGATGATTATTATTGCCAGTCCTATGCCAGCAGCGTGAGTGGTGTGATATT | | | | | |
| | CGGCGGAGGGACCAAGCTGACCGTCCTA | | | | | |
| ADI-75716 | GACATCCAGATGACCCAGTCTCCATCCTTCCTGTCTGCATCTGTAGGAGACAGAGTCACCA | 3135 | DIQMTQSPSFLSASVGDRVTITCRASQG | 3417 | | |
| | TCACTTGCCGGGCCAGTCAGGGCATTAGCAATTATTTAGCCTGGTATCAGCAAAAACCAGG | | ISNYLAWYQQKPGKAPKLLIYAASTLQS | | | |
| | GAAAGCCCCTAAGCTCCTGATCTATGCTGCATCCACTTTGCAAAGTGGGGTCCCATCAAGG | | GVPSRFSGGGSGTEFTLTISSLQPEDFA | | | |
| | TTCAGCGGCGGTGGATCTGGGACAGAATTCACTCTCACAATCAGCAGCCTGCAGCCTGAAG | | TYYCQQLNSNPPITFGQGTRLEIK | | | |
| | ATTTTGCAACTTATTACTGTCAACAGCTTAATAGTAACCCCCCTATCACCTTCGGCCAAGG | | | | | |
| | GACACGACTGGAGATTAAA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75689 | IGLV3-21 | SHVLTQ | 1988 | GGNNIGS | 2188 | WYQQKP | 2340 | DDSDRP | 2399 | GIPERFSG | 2643 | QVWDS | 2846 | FGTGTK | 2945 |
| | | PPSVSV | | KNVH | | GQAPEL | | S | | SNSGNTAT | | TTDLL | | VTVL | |
| | | APGQTA | | | | VVY | | | | LTISRVEA | | YV | | | |
| | | RITC | | | | | | | | GDEADYFC | | | | | |
| ADI-75669 | IGLV1-36 | QSVLTQ | 1989 | GNNAVN | 2189 | WYQQFP | 2341 | YDDLLP | 2433 | GVSDRFSG | 2578 | AAWDD | 2847 | FGGGTK | 2939 |
| | | PPSVSE | | SGSSSNI | | GKAPKL | | S | | SKSGTSAS | | RLNGV | | LTVL | |
| | | APRQRV | | | | LIY | | | | LAISGLQS | | I | | | |
| | | TISC | | | | | | | | EDEADYYC | | | | | |
| ADI-75839 | IGKV1-5 | DIQMTQ | 1879 | RASQSIS | 2190 | WYQQKP | 2257 | KSSSLE | 2480 | GVPSRFSG | 2532 | QQYNS | 2798 | FGQGTK | 2937 |
| | | SPSTLS | | SWLA | | GKAPKL | | S | | SGSGTEFT | | YYT | | VEIK | |
| | | ASVGDR | | | | LIY | | | | LTISSLQP | | | | | |
| | | VTITC | | | | | | | | DDFATYYC | | | | | |
| ADI-75754 | IGKV3-20 | EMTMTQ | 1990 | RASESVS | 2191 | WHQQRP | 2342 | GASSRP | 2481 | GIPDRFSG | 2616 | HQFAS | 2848 | FGQGTR | 2940 |
| | | SPGTLS | | SEYLA | | GQAPRL | | T | | SGSGTDFT | | APIT | | | |
| | | LSPGER | | | | LIY | | | | LTISRLEP | | | | LEIK | |
| | | ATLSC | | | | | | | | EDFAVYYC | | | | | |
| ADI-75743 | IGKV3-20 | ETTLTQ | 1991 | RASESVR | 2192 | WYQQSP | 2343 | GA- | 2482 | GIPDRFSG | 2644 | QQYAR | 2849 | FGQGTK | 2937 |
| | | SPGTLV | | SSYLA | | GQTPRL | | SRAT | | SGSGTDFT | | SPWT | | VEIK | |
| | | LSPGER | | | | LIY | | | | LTITRLEP | | | | | |
| | | ATLSC | | | | | | | | EDFAVYYC | | | | | |
| ADI-75841 | IGLV6-57 | NFMLTQ | 1992 | TRSSGSL | 2193 | WYQLRP | 2344 | EDNQRP | 2431 | GVPDRFSG | 2574 | QSWDS | 2850 | FGGGTK | 2939 |
| | | PHSVSE | | ATNYVQ | | GSSPST | | S | | SIDSSSNS | | SALWV | | LTVL | |
| | | SPGKTV | | | | VIY | | | | ASLTISGL | | | | | |
| | | TISC | | | | | | | | KTEDEADY | | | | | |
| ADI-75879 | IGKV3-20 | EIAMTQ | 1993 | RASESVS | 2194 | WYQQKP | 2258 | GASSRA | 2464 | GIPDRFSG | 2645 | QQYGS | 2851 | FGPGTK | 2942 |
| | | SPGTLS | | SSYLA | | GQAPRL | | T | | SGSGTDFT | | SPV | | VEIK | |
| | | LSPGQR | | | | LIY | | | | LTISRLEP | | | | | |
| | | ATLSC | | | | | | | | EDYAVYYC | | | | | |
| ADI-75703 | IGKV1D- | DIPMTQ | 1994 | RASQSIS | 2195 | WYQQKP | 2345 | IASNLQ | 2483 | GVPSRFSG | 2534 | QQSYT | 2852 | FGGGTK | 2936 |
| | 39 | SPSSLS | | NFLN | | GRAPNL | | R | | SGSGTDFT | | TPPT | | VEIK | |
| | | ASVGDS | | | | LIY | | | | LTISSLQP | | | | | |
| | | VTITC | | | | | | | | EDFATYYC | | | | | |
| ADI-75739 | IGLV1-40 | QSVLTQ | 1948 | TGSNSNI | 2196 | WYQQLP | 2314 | GNTNRP | 2484 | GVPDRFSG | 2622 | QSYDS | 2853 | FGGGTK | 2947 |
| | | PPSVSG | | GAGYDIH | | GTAPKL | | S | | SKSDTSAS | | SLSGV | | VTVL | |
| | | APGQRV | | | | LIY | | | | LAITGLQA | | V | | | |
| | | TISC | | | | | | | | EDEADYYC | | | | | |
| ADI-75762 | IGLV2-11 | QSALTQ | 1995 | TGTSSDV | 2061 | WYQQHP | 2265 | DVSKRP | 2409 | GVPDRFSG | 2646 | CSYVG | 2854 | FGTGTK | 2949 |
| | | PRSVSG | | GGYNYVS | | GKAPKL | | S | | SKSGNTAS | | SYSYV | | LTVL | |
| | | SPGQSV | | | | MIY | | | | LTMSGLQA | | | | | |
| | | TISC | | | | | | | | EDEADYYC | | | | | |
| ADI-75815 | IGKV3-11 | EIVMTQ | 1996 | RASQSVS | 2168 | WYQQKP | 2258 | DASNRA | 2448 | GIPARFTG | 2647 | QQRIH | 2855 | FGPGTK | 2942 |
| | | SPATLS | | SYLA | | GQAPRL | | T | | SGSGTDFT | | PPFT | | VEIK | |
| | | LSPGER | | | | LIY | | | | LTISSLEP | | | | | |
| | | ATLSC | | | | | | | | EDFAVYYC | | | | | |
| ADI-75765 | IGKV1-16 | DIQLTQ | 1876 | RASQGIS | 2187 | WFQQKP | 2346 | DASTLQ | 2485 | GVPSKFSG | 2648 | QQYDS | 2856 | FGGGTK | 2936 |
| | | SPSSLS | | NYLA | | GKAPKS | | N | | SGSGTHFT | | YPLT | | VEIK | |
| | | ASVGDR | | | | LIY | | | | LTISSLQP | | | | | |
| | | VTITC | | | | | | | | EDFATYYC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75689 | AGCCATGTGCTGACTCAGCCACCCTCGGTGTCAGTGGCCCCAGGACAGACGGCCAGGATTA | 3136 | SHVLTQPPSVSVAPGQTARITCGGNNIG | 3418 | | |
| | CCTGTGGGGGAAACAACATTGGAAGTAAAAATGTGCACTGGTACCAGCAGAAGCCAGGCCA | | SKNVHWYQQKPGQAPELVVYDDSDRPSG | | | |
| | GGCCCCTGAACTGGTCGTCTATGACGATAGCGACCGGCCCTCAGGGATCCCTGAGCGATTC | | IPERFSGSNSGNTATLTISRVEAGDEAD | | | |
| | TCTGGCTCCAACTCTGGGAACACGGCCACCCTGACCATCAGCAGGGTCGAAGCCGGGGATG | | YFCQVWDSTTDLLYVFGTGTKVTVL | | | |
| | AGGCCGACTACTTCTGTCAGGTGTGGGATAGTACTACTGATCTTCTTTATGTCTTCGGAAC | | | | | |
| | TGGGACCAAGGTCACCGTCCTA | | | | | |
| ADI-75669 | CAGTCTGTGCTGACTCAGCCCCCCTCGGTGTCTGAAGCCCCCAGGCAGAGGGTCACCATCT | 3137 | QSVLTQPPSVSEAPRQRVTISCSGSSSN | 3419 | | |
| | CCTGTTCTGGAAGCAGCTCCAACATCGGAAATAATGCTGTAAACTGGTACCAGCAGTTCCC | | IGNNAVNWYQQFPGKAPKLLIYYDDLLP | | | |
| | AGGAAAGGCTCCCAAACTCCTCATCTATTATGATGATCTGCTGCCCTCAGGGGTCTCTGAC | | SGVSDRFSGSKSGTSASLAISGLQSEDE | | | |
| | CGATTCTCTGGCTCCAAGTCTGGCACCTCAGCCTCCCTGGCCATCAGTGGGCTCCAGTCTG | | ADYYCAAWDDRLNGVIFGGGTKLTVL | | | |
| | AGGATGAGGCTGATTATTACTGTGCAGCATGGGATGACAGGCTCAATGGTGTGATCTTCGG | | | | | |
| | CGGGGGGACCAAGCTCACCGTCCTA | | | | | |
| ADI-75839 | GACATCCAGATGACCCAGTCTCCTTCCACCCTGTCTGCATCTGTAGGAGACAGAGTCACCA | 3138 | DIQMTQSPSTLSASVGDRVTITCRASQS | 3420 | | |
| | TCACTTGCCGGGCCAGTCAGAGTATTAGTAGCTGGTTGGCCTGGTATCAGCAGAAGCCAGG | | ISSWLAWYQQKPGKAPKLLIYKSSSLES | | | |
| | GAAAGCCCCTAAGCTCCTGATCTATAAGTCATCTAGTTTAGAAAGTGGGGTCCCATCAAGG | | GVPSRFSGSGSGTEFTLTISSLQPDDFA | | | |
| | TTCAGCGGCAGTGGATCTGGGACAGAATTCACTCTCACCATCAGCAGCCTGCAGCCTGATG | | TYYCQQYNSYYTFGQGTKVEIK | | | |
| | ATTTTGCAACTTATTACTGCCAACAGTATAATAGTTATTACACTTTTGGCCAGGGGACCAA | | | | | |
| | GGTGGAAATCAAA | | | | | |
| ADI-75754 | GAAATGACAATGACGCAGTCTCCAGGCACCCTGTCTTTGTCTCCAGGGGAAAGAGCCACCC | 3139 | EMTMTQSPGTLSLSPGERATLSCRASES | 3421 | | |
| | TCTCCTGCAGGGCCAGTGAGAGCGTTAGCAGCGAGTACCTAGCCTGGCACCAGCAGAGACC | | VSSEYLAWHQQRPGQAPRLLIYGASSRP | | | |
| | TGGCCAGGCTCCCAGGCTCCTCATCTATGGTGCATCCAGCAGGCCCACGGGCATCCCAGAC | | TGIPDRFSGSGSGTDFTLTISRLEPEDF | | | |
| | AGGTTCAGTGGCAGTGGGTCTGGGACAGACTTCACTCTCACCATCAGCAGACTGGAGCCTG | | AVYYCHQFASAPITFGQGTRLEIK | | | |
| | AAGATTTTGCAGTGTATTACTGTCACCAGTTTGCTAGCGCACCGATCACCTTCGGCCAAGG | | | | | |
| | GACACGACTGGAGATTAAA | | | | | |
| ADI-75743 | GAAACGACACTCACGCAGTCTCCAGGCACCCTGGTTTTGTCTCCAGGGGAAAGAGCCACCC | 3140 | ETTLTQSPGTLVLSPGERATLSCRASES | 3422 | | |
| | TCTCCTGCAGGGCCAGTGAGAGTGTTCGGAGCAGCTATTTAGCCTGGTACCAGCAGAGTCC | | VRSSYLAWYQQSPGQTPRLLIYGASRAT | | | |
| | TGGCCAGACTCCCAGGCTCCTCATCTATGGTGCATCCAGGGCCACTGGCATCCCAGACAGG | | GIPDRFSGSGSGTDFTLTITRLEPEDFA | | | |
| | TTCAGTGGTAGTGGGTCTGGGACAGACTTCACTCTCACCATCACCAGATTGGAGCCTGAAG | | VYYCQQYARSPWTFGQGTKVEIK | | | |
| | ATTTTGCAGTGTATTACTGTCAGCAGTATGCTAGGTCACCGTGGACGTTCGGCCAAGGGAC | | | | | |
| | CAAGGTGGAGATCAAA | | | | | |
| ADI-75841 | AATTTTATGCTGACTCAGCCCCACTCTGTGTCGGAGTCTCCGGGGAAGACGGTAACCATCT | 3141 | NFMLTQPHSVSESPGKTVTISCTRSSGS | 3423 | | |
| | CCTGCACCCGCAGTAGTGGCAGCCTTGCCACCAACTATGTGCAGTGGTACCAGCTGCGCCC | | LATNYVQWYQLRPGSSPSTVIYEDNQRP | | | |
| | GGGCAGTTCCCCCAGCACTGTGATCTATGAGGATAACCAAAGACCCTCTGGGGTCCCTGAT | | SGVPDRFSGSIDSSSNSASLTISGLKTE | | | |
| | CGGTTCTCTGGCTCCATCGACAGCTCCTCCAACTCTGCCTCCCTCACCATCTCTGGACTGA | | DEADYYCQSWDSSALWVFGGGTKLTVL | | | |
| | AGACTGAGGACGAGGCTGACTACTACTGTCAGTCTTGGGATAGCAGCGCTCTTTGGGTGTT | | | | | |
| | CGGCGGAGGGACCAAGCTCACCGTCCTA | | | | | |
| ADI-75879 | GAAATTGCGATGACCCAGTCTCCAGGCACCCTGTCTTTGTCTCCAGGGCAAAGAGCCACCC | 3142 | EIAMTQSPGTLSLSPGQRATLSCRASES | 3424 | | |
| | TCTCCTGCAGGGCCAGTGAGAGTGTTAGCAGCAGCTACTTAGCCTGGTACCAGCAGAAACC | | VSSSYLAWYQQKPGQAPRLLIYGASSRA | | | |
| | TGGCCAGGCTCCCAGGCTCCTCATCTATGGTGCATCCAGCAGGGCCACTGGCATCCCAGAC | | TGIPDRFSGSGSGTDFTLTISRLEPEDY | | | |
| | AGGTTCAGTGGCAGTGGGTCTGGGACAGACTTCACTCTCACCATCAGCAGACTGGAGCCTG | | AVYYCQQYGSSPVFGPGTKVEIK | | | |
| | AAGATTATGCAGTATATTACTGTCAGCAGTATGGTAGCTCACCTGTTTTCGGCCCTGGGAC | | | | | |
| | CAAAGTGGAAATCAAA | | | | | |
| ADI-75703 | GACATCCCGATGACCCAGTCTCCATCCTCCCTGTCTGCTTCTGTAGGAGACAGCGTCACCA | 3143 | DIPMTQSPSSLSASVGDSVTITCRASQS | 3425 | | |
| | TCACTTGCCGGGCAAGTCAGAGCATTAGCAACTTTTTAAATTGGTATCAGCAGAAACCAGG | | ISNFLNWYQQKPGRAPNLLIYIASNLQR | | | |
| | GAGAGCCCCTAACCTCCTGATCTACATTGCATCCAATTTGCAACGTGGGGTCCCATCAAGG | | GVPSRFSGSGSGTDFTLTISSLQPEDFA | | | |
| | TTCAGTGGCAGTGGGTCTGGGACAGATTTCACTCTCACCATCAGCAGTCTGCAACCTGAAG | | TYYCQQSYTTPPTFGGGTKVEIK | | | |
| | ATTTTGCAACTTACTACTGTCAACAGAGTTACACTACTCCTCCGACTTTCGGCGGAGGGAC | | | | | |
| | CAAGGTGGAGATCAAA | | | | | |
| ADI-75739 | CAGTCTGTCTTGACGCAGCCGCCCTCAGTGTCTGGGGCCCCAGGGCAGAGGGTCACCATCT | 3144 | QSVLTQPPSVSGAPGQRVTISCTGSNSN | 3426 | | |
| | CCTGCACTGGGAGCAACTCCAACATCGGAGCAGGTTATGATATACACTGGTACCAGCAGCT | | IGAGYDIHWYQQLPGTAPKLLIYGNTNR | | | |
| | TCCAGGAACGGCCCCCAAACTCCTCATCTATGGTAACACCAATCGGCCCTCAGGGGTCCCT | | PSGVPDRFSGSKSDTSASLAITGLQAED | | | |
| | GACCGATTCTCTGGCTCCAAGTCTGACACCTCAGCCTCCCTGGCCATCACTGGGCTCCAGG | | EADYYCQSYDSSLSGVVFGGGTKVTVL | | | |
| | CTGAGGATGAGGCTGATTATTACTGCCAGTCTTATGACAGCAGCCTGAGTGGTGTGGTATT | | | | | |
| | CGGCGGAGGGACCAAGGTGACCGTCCTA | | | | | |
| ADI-75762 | CAGTCTGCGCTGACTCAGCCTCGCTCAGTGTCCGGGTCTCCTGGACAGTCAGTCACCATCT | 3145 | QSALTQPRSVSGSPGQSVTISCTGTSSD | 3427 | | |
| | CCTGCACTGGAACCAGCAGTGATGTTGGTGGTTATAACTATGTCTCCTGGTACCAACAACA | | VGGYNYVSWYQQHPGKAPKLMIYDVSKR | | | |
| | CCCAGGCAAAGCCCCCAAACTCATGATTTATGATGTCAGTAAGCGGCCCTCAGGGGTCCCT | | PSGVPDRFSGSKSGNTASLTMSGLQAED | | | |
| | GATCGCTTCTCTGGCTCCAAGTCTGGCAACACGGCCTCCCTGACCATGTCTGGGCTCCAGG | | EADYYCCSYVGSYSYVFGTGTKLTVL | | | |
| | CTGAGGATGAGGCTGATTATTACTGCTGCTCATATGTAGGCAGCTATAGTTATGTCTTCGG | | | | | |
| | AACTGGGACCAAGCTCACCGTCCTA | | | | | |
| ADI-75815 | GAAATTGTGATGACGCAGTCTCCAGCCACCCTGTCTTTGTCTCCAGGGGAAAGAGCCACCC | 3146 | EIVMTQSPATLSLSPGERATLSCRASQS | 3428 | | |
| | TCTCCTGCAGGGCCAGTCAGAGTGTTAGCAGCTACTTAGCCTGGTACCAACAGAAACCTGG | | VSSYLAWYQQKPGQAPRLLIYDASNRAT | | | |
| | CCAGGCTCCCAGGCTCCTCATCTATGATGCTTCCAACAGGGCCACTGGCATCCCAGCCAGG | | GIPARFTGSGSGTDFTLTISSLEPEDFA | | | |
| | TTCACTGGCAGTGGGTCTGGGACAGACTTCACTCTCACCATCAGCAGCCTAGAGCCTGAAG | | VYYCQQRIHPPFTFGPGTKVEIK | | | |
| | ATTTTGCAGTTTATTACTGTCAGCAACGTATCCACCCGCCATTCACTTTCGGCCCTGGGAC | | | | | |
| | CAAGGTGGAGATCAAA | | | | | |
| ADI-75765 | GACATCCAGTTGACCCAGTCTCCATCCTCACTGTCTGCATCTGTAGGAGACAGAGTCACCA | 3147 | DIQLTQSPSSLSASVGDRVTITCRASQG | 3429 | | |
| | TCACTTGTCGGGCGAGTCAGGGCATTAGCAATTATTTAGCCTGGTTTCAACAGAAACCAGG | | ISNYLAWFQQKPGKAPKSLIYDASTLQN | | | |
| | GAAAGCCCCTAAGTCCCTCATCTATGATGCATCCACTTTGCAAAATGGGGTCCCATCAAAG | | GVPSKFSGSGSGTHFTLTISSLQPEDFA | | | |
| | TTCAGCGGCAGTGGATCTGGGACACATTTCACTCTCACCATCAGCAGCCTGCAGCCTGAAG | | TYYCQQYDSYPLTFGGGTKVEIK | | | |
| | ATTTTGCAACTTATTACTGCCAACAATATGATAGTTACCCGCTCACTTTCGGCGGAGGGAC | | | | | |
| | CAAGGTGGAAATCAAA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75705 | IGKV3-20 | EMQLTQ | 1997 | RASQSLN | 2197 | WYQQKP | 2258 | AASSRA | 2486 | GIPDRFSG | 2649 | QQYGY | 2857 | FGGGTK | 2938 |
| | | TPGTLS | | SKYFA | | GQAPRL | | T | | SGYGTDFT | | SPAT | | VDIK | |
| | | LSPGEG | | | | LIY | | | | LTITRLEP | | | | | |
| | | ATVSC | | | | | | | | EDFAVYFC | | | | | |
| ADI-75678 | IGKV1D- | DIQMTQ | 1878 | RASQSIS | 2090 | WYQQKP | 2257 | AASSLQ | 2391 | GVPSRVSG | 2650 | QQSYS | 2858 | FGQGTK | 2937 |
| | 39 | SPSSLS | | SFLN | | GKAPKL | | S | | SGSGTEFT | | SPMYT | | VEIK | |
| | | ASVGDR | | | | LIY | | | | LTISSLQP | | | | | |
| | | VTITC | | | | | | | | DDFATYYC | | | | | |
| ADI-75707 | IGKV1D- | EIPMTQ | 1998 | RASQSIS | 2153 | WYQQKP | 2347 | AASNLQ | 2408 | GVPSRFSG | 2534 | QQSYS | 2859 | FGQGTK | 2937 |
| | 39 | SPSSLS | | ITFLN | | GKAPNL | | S | | SGSGTDFT | | SPCS | | VEIK | |
| | | ASVGDT | | | | LVY | | | | LTISSLQP | | | | | |
| | | VTITC | | | | | | | | EDFATYYC | | | | | |
| ADI-75796 | IGLV6-57 | NLMLTQ | 1999 | ASYYVQ | 2198 | WYQHRP | 2348 | EDNQRP | 2431 | GVPDRFSG | 2574 | HSYDS | 2860 | FGGGTK | 2939 |
| | | PHSVSA | | TRSGGSI | | GSAPTI | | S | | SIDSSSNS | | SNPVV | | LTVL | |
| | | SPGKTV | | | | VIY | | | | ASLTISGL | | | | | |
| | | TISC | | | | | | | | KTEDEADY | | | | | |
| | | | | | | | | | | YC | | | | | |
| ADI-75673 | IGLV2-11 | QPVLTQ | 2000 | TGTTSDV | 2199 | WYPPHP | 2349 | DVSKRP | 2409 | GVPDRFSG | 2651 | CSYAG | 2861 | FGTGTK | 2949 |
| | | PRSVSG | | GSYNYVS | | GKAPKL | | S | | SKSGNTAS | | TYI | | LTVL | |
| | | SPGQSV | | | | MIY | | | | LTISGLQA | | | | | |
| | | TISC | | | | | | | | EDEADYYC | | | | | |
| ADI-75566 | IGKV1-33 | EIQLTQ | 1917 | QASQDIN | 2160 | WYQQKP | 2257 | GATNLE | 2487 | GVPSRFSG | 2652 | QYYDN | 2799 | FGPGTK | 2942 |
| | | SPSSLS | | KYLN | | GKAPKL | | T | | SGSGTDFS | | PPPT | | VEIK | |
| | | ASVGDR | | | | LIY | | | | FTINSLQP | | | | | |
| | | VTITC | | | | | | | | EDIATYYC | | | | | |
| ADI-75706 | IGLV1-40 | QSVLTQ | 1948 | TGSNSNI | 2196 | WYQQLP | 2314 | ANTNRP | 2488 | GVPDRFSG | 2622 | QSYDS | 2862 | FGGGTK | 2947 |
| | | PPSVSG | | GAGYDIH | | GTAPKL | | S | | SKSDTSAS | | SLSGV | | VTVL | |
| | | APGQRV | | | | LIY | | | | LAITGLQA | | L | | | |
| | | TISC | | | | | | | | EDEADYYC | | | | | |
| ADI-75699 | IGLV1-40 | QPVLTQ | 2001 | TGSNSNI | 2143 | WYQQLP | 2314 | DNNNRP | 2476 | GVPDRFSG | 2622 | QSYDS | 2840 | FGYGTK | 2956 |
| | | PPSVSA | | GAGYDVH | | GTAPKL | | S | | SKSDTSAS | | SLSDS | | LTVL | |
| | | APGQRV | | | | LIY | | | | LAITGLQA | | V | | | |
| | | TISC | | | | | | | | EDEADYYC | | | | | |
| ADI-75591 | IGKV3D- | EIVLTQ | 2002 | RASQSIN | 2200 | WFQQKP | 2350 | GASARA | 2489 | GIPARFGG | 2653 | LQYHN | 2863 | FGQGTK | 2937 |
| | 15 | SPATLS | | SDLA | | GQAPRL | | A | | SGSGTEFT | | RPPWT | | VEIK | |
| | | VSPGER | | | | LMF | | | | LTISGLQS | | | | | |
| | | ASLSC | | | | | | | | EDFAVYYC | | | | | |
| ADI-75698 | IGLV1-40 | QSVLTQ | 1948 | TGSSSNI | 2201 | WYQQLP | 2314 | GNTHRP | 2490 | GVPDRFSG | 2599 | QSYDS | 2864 | FGGGTK | 2939 |
| | | PPSVSG | | GAGYDVH | | GTAPKL | | S | | SKSGTSAS | | SLIGS | | LTVL | |
| | | APGQRV | | | | LIY | | | | LAITGLQA | | V | | | |
| | | TISC | | | | | | | | EDEADYYC | | | | | |
| ADI-75878 | IGKV3D- | EMTMTQ | 2003 | RASQSVS | 2202 | WYQQKP | 2258 | GASTRA | 2383 | GIPARFSG | 2569 | QQYDN | 2865 | FGQGTK | 2937 |
| | 15 | SPATLS | | TNLA | | GQAPRL | | T | | SGSGTEFT | | WPPTW | | VEIK | |
| | | VSPGER | | | | LIY | | | | LTISSLQS | | T | | | |
| | | ATLSC | | | | | | | | EDFAVYYC | | | | | |
| ADI-75718 | IGKV3D- | EMTLTQ | 2004 | RASQSLS | 2203 | WYQQRP | 2351 | GASGRA | 2491 | GLPDRFSG | 2654 | QQYDS | 2866 | FGQGTK | 2937 |
| | 15 | SPATLS | | NKLA | | GQAPTL | | T | | SGYGTEFT | | WPRT | | VEIK | |
| | | VSPGET | | | | LIH | | | | LTIRRLQS | | | | | |
| | | VTLSC | | | | | | | | EDFAVYYC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75705 | GAAATGCAACTCACGCAGACTCCAGGCACCCTGTCTTTGTCTCCAGGAGAAGGAGCCACCG | 3148 | EMQLTQTPGTLSLSPGEGATVSCRASQS | 3430 | | |
| | TCTCCTGCAGGGCCAGTCAGAGTCTTAACAGCAAGTACTTCGCCTGGTACCAGCAGAAACC | | LNSKYFAWYQQKPGQAPRLLIYAASSRA | | | |
| | GGGCCAGGCTCCCAGGCTCCTCATCTATGCTGCGTCCAGCAGGGCCACTGGCATCCCAGAC | | TGIPDRFSGSGYGTDFTLTITRLEPEDF | | | |
| | AGGTTCAGTGGCAGTGGGTATGGGACAGACTTCACTCTCACCATCACCAGACTGGAGCCTG | | AVYFCQQYGYSPATFGGGTKVDIK | | | |
| | AAGATTTTGCAGTTTATTTCTGTCAGCAATATGGTTACTCACCGGCCACTTTCGGCGGAGG | | | | | |
| | GACCAAAGTGGATATCAAA | | | | | |
| ADI-75678 | GACATCCAGATGACCCAGTCTCCATCCTCCCTGTCTGCCTCTGTAGGAGACAGAGTCACCA | 3149 | DIQMTQSPSSLSASVGDRVTITCRASQS | 3431 | | |
| | TCACTTGCCGGGCAAGTCAGAGCATTAGCAGCTTTTTAAATTGGTATCAGCAGAAACCGGG | | ISSFLNWYQQKPGKAPKLLIYAASSLQS | | | |
| | GAAAGCCCCTAAGCTCCTGATTTATGCTGCATCCAGTTTGCAAAGTGGGGTCCCATCAAGG | | GVPSRVSGSGSGTEFTLTISSLQPDDFA | | | |
| | GTCAGTGGCAGTGGATCTGGGACAGAATTCACTCTCACCATCAGCAGCCTGCAACCTGACG | | TYYCQQSYSSPMYTFGQGTKVEIK | | | |
| | ATTTTGCAACTTACTACTGTCAACAGAGTTACAGTAGCCCCATGTACACTTTTGGCCAGGG | | | | | |
| | GACCAAGGTGGAGATCAAA | | | | | |
| ADI-75707 | GAAATCCCGATGACCCAGTCTCCATCCTCCCTGTCTGCATCTGTAGGAGACACAGTCACCA | 3150 | EIPMTQSPSSLSASVGDTVTITCRASQS | 3432 | | |
| | TCACTTGCCGGGCAAGTCAGAGCATTAGCACCTTTTTAAATTGGTATCAGCAGAAACCAGG | | ISTFLNWYQQKPGKAPNLLVYAASNLQS | | | |
| | GAAAGCCCCTAACCTCCTCGTCTATGCTGCATCCAATTTGCAAAGTGGGGTCCCATCAAGG | | GVPSRFSGSGSGTDFTLTISSLQPEDFA | | | |
| | TTCAGTGGCAGTGGATCTGGGACAGATTTCACTCTCACCATCAGCAGTCTGCAACCTGAAG | | TYYCQQSYSSPCSFGQGTKVEIK | | | |
| | ATTTTGCAACTTACTACTGTCAACAGAGTTACAGTTCCCCGTGCAGTTTTGGCCAGGGGAC | | | | | |
| | CAAGGTGGAAATCAAA | | | | | |
| ADI-75796 | AATCTTATGCTGACTCAGCCCCACTCTGTGTCGGCGTCTCCGGGGAAGACGGTAACCATCT | 3151 | NLMLTQPHSVSASPGKTVTISCTRSGGS | 3433 | | |
| | CCTGCACCCGCAGCGGTGGCAGTATTGCCAGCTACTATGTGCAGTGGTACCAGCACCGCCC | | IASYYVQWYQHRPGSAPTIVIYEDNQRP | | | |
| | GGGCAGTGCCCCCACCATTGTGATTTATGAGGATAACCAAAGACCCTCTGGGGTCCCTGAT | | SGVPDRFSGSIDSSSNSASLTISGLKTE | | | |
| | CGGTTCTCTGGCTCCATCGACAGCTCCTCCAACTCCGCCTCCCTCACCATCTCTGGACTGA | | DEADYYCHSYDSSNPVVFGGGTKLTVL | | | |
| | AGACTGAGGACGAGGCTGACTACTACTGTCACTCTTATGACAGCAGCAATCCTGTGGTCTT | | | | | |
| | CGGCGGAGGGACCAAGCTCACCGTCCTA | | | | | |
| ADI-75673 | CAGCCTGTGCTGACTCAGCCTCGCTCAGTGTCCGGGTCTCCTGGACAGTCAGTCACCATCT | 3152 | QPVLTQPRSVSGSPGQSVTISCTGTTSD | 3434 | | |
| | CCTGCACTGGAACCACCAGTGATGTTGGTAGTTATAACTATGTCTCCTGGTACCCCCCGCA | | VGSYNYVSWYPPHPGKAPKLMIYDVSKR | | | |
| | CCCAGGCAAAGCCCCCAAACTCATGATTTATGATGTCAGTAAGCGGCCCTCAGGGGTCCCT | | PSGVPDRFSGSKSGNTASLTISGLQAED | | | |
| | GATCGCTTCTCTGGCTCCAAGTCTGGCAACACGGCCTCCCTGACCATCTCTGGGCTCCAGG | | EADYYCCSYAGTYIFGTGTKLTVL | | | |
| | CTGAGGATGAGGCTGATTATTACTGCTGTTCATATGCAGGCACCTACATCTTCGGAACTGG | | | | | |
| | GACCAAGCTCACCGTCCTA | | | | | |
| ADI-75566 | GAAATCCAATTGACCCAGTCTCCATCCTCCCTGTCTGCATCTGTAGGAGACAGAGTCACCA | 3153 | EIQLTQSPSSLSASVGDRVTITCQASQD | 3435 | | |
| | TCACTTGCCAGGCGAGTCAGGACATTAACAAGTATTTAAATTGGTATCAGCAGAAACCAGG | | INKYLNWYQQKPGKAPKLLIYGATNLET | | | |
| | GAAAGCCCCTAAGCTCCTGATCTACGGTGCAACCAATTTGGAGACAGGGGTCCCATCAAGG | | GVPSRFSGSGSGTDFSFTINSLQPEDIA | | | |
| | TTCAGTGGAAGTGGATCTGGGACAGATTTTAGTTTCACCATCAACAGCCTGCAGCCTGAAG | | TYYCQYYDNPPPTFGPGTKVEIK | | | |
| | ATATTGCAACATATTACTGTCAATATTATGATAATCCCCCTCCGACTTTCGGCCCTGGGAC | | | | | |
| | CAAGGTGGAGATCAAA | | | | | |
| ADI-75706 | CAGTCTGTGTTGACGCAGCCGCCCTCAGTGTCTGGGGCCCCAGGGCAGAGGGTCACCATCT | 3154 | QSVLTQPPSVSGAPGQRVTISCTGSNSN | 3436 | | |
| | CCTGCACTGGGAGCAACTCCAACATCGGAGCAGGTTATGATATACACTGGTACCAGCAGCT | | IGAGYDIHWYQQLPGTAPKLLIYANTNR | | | |
| | TCCAGGAACAGCCCCCAAACTCCTCATCTATGCTAACACCAATCGGCCCTCAGGGGTCCCT | | PSGVPDRFSGSKSDTSASLAITGLQAED | | | |
| | GACCGATTCTCTGGCTCCAAGTCTGACACCTCAGCCTCCCTGGCCATCACTGGGCTCCAGG | | EADYYCQSYDSSLSGVLFGGGTKVTVL | | | |
| | CTGAGGATGAGGCTGATTATTACTGCCAGTCTTATGACAGCAGCCTGAGTGGTGTGCTATT | | | | | |
| | CGGCGGAGGGACCAAGGTCACCGTCCTA | | | | | |
| ADI-75699 | CAGCCTGTGTTGACGCAGCCGCCCTCAGTGTCTGCGGCCCCAGGGCAGAGGGTCACCATCT | 3155 | QPVLTQPPSVSAAPGQRVTISCTGSNSN | 3437 | | |
| | CCTGCACTGGGAGCAACTCCAACATCGGGGCAGGTTATGATGTACACTGGTACCAGCAACT | | IGAGYDVHWYQQLPGTAPKLLIYDNNNR | | | |
| | TCCAGGAACAGCCCCCAAACTCCTCATCTATGATAACAACAATCGGCCCTCAGGGGTCCCT | | PSGVPDRFSGSKSDTSASLAITGLQAED | | | |
| | GACCGATTCTCTGGCTCCAAGTCTGACACCTCAGCCTCCCTGGCCATCACTGGGCTCCAGG | | EADYYCQSYDSSLSDSVFGYGTKLTVL | | | |
| | CTGAGGATGAGGCTGATTATTACTGCCAGTCCTATGACAGCAGCCTGAGTGATTCTGTCTT | | | | | |
| | CGGATATGGGACCAAGCTGACCGTCCTA | | | | | |
| ADI-75591 | GAAATTGTGTTGACGCAGTCTCCAGCCACACTGTCTGTGTCTCCAGGGGAAAGAGCCTCCC | 3156 | EIVLTQSPATLSVSPGERASLSCRASQS | 3438 | | |
| | TCTCCTGCAGGGCCAGTCAGAGTATTAACAGCGACTTAGCCTGGTTCCAGCAGAAACCTGG | | INSDLAWFQQKPGQAPRLLMFGASARAA | | | |
| | CCAGGCTCCCAGGCTCCTCATGTTTGGTGCGTCTGCCAGGGCCGCTGGTATCCCAGCCAGG | | GIPARFGGSGSGTEFTLTISGLQSEDFA | | | |
| | TTCGGTGGCAGTGGGTCTGGGACAGAGTTCACTCTCACCATCAGCGGCCTGCAGTCTGAAG | | VYYCLQYHNRPPWTFGQGTKVEIK | | | |
| | ATTTTGCAGTTTATTACTGTCTGCAGTATCATAACAGGCCTCCGTGGACGTTCGGCCAAGG | | | | | |
| | GACCAAGGTGGAAATCAAA | | | | | |
| ADI-75698 | CAGTCTGTGTTGACGCAGCCGCCCTCAGTGTCTGGGGCCCCAGGGCAGAGGGTCACAATCT | 3157 | QSVLTQPPSVSGAPGQRVTISCTGSSSN | 3439 | | |
| | CCTGCACTGGGAGCAGCTCCAACATCGGGGCAGGTTATGATGTACACTGGTACCAGCAGCT | | IGAGYDVHWYQQLPGTAPKLLIYGNTHR | | | |
| | TCCAGGAACAGCCCCCAAACTCCTCATTTATGGTAACACCCATCGGCCCTCAGGGGTCCCT | | PSGVPDRFSGSKSGTSASLAITGLQAED | | | |
| | GACCGATTCTCTGGCTCCAAGTCTGGCACCTCAGCCTCCCTGGCCATCACTGGACTCCAGG | | EADYYCQSYDSSLIGSVFGGGTKLTVL | | | |
| | CTGAGGATGAGGCTGATTATTACTGCCAGTCCTATGACAGCAGCCTGATTGGTTCGGTGTT | | | | | |
| | CGGCGGAGGGACCAAGCTCACCGTCCTA | | | | | |
| ADI-75878 | GAAATGACAATGACGCAGTCTCCAGCCACCCTGTCTGTGTCTCCAGGGGAAAGAGCCACCC | 3158 | EMTMTQSPATLSVSPGERATLSCRASQS | 3440 | | |
| | TCTCCTGCAGGGCCAGTCAGAGTGTTAGCACCAACTTAGCCTGGTACCAGCAGAAACCTGG | | VSTNLAWYQQKPGQAPRLLIYGASTRAT | | | |
| | CCAGGCTCCCAGGCTCCTCATCTATGGTGCATCCACCAGGGCCACTGGTATCCCAGCCAGG | | GIPARFSGSGSGTEFTLTISSLQSEDFA | | | |
| | TTCAGTGGCAGTGGGTCTGGGACAGAGTTCACTCTCACCATCAGCAGCCTGCAGTCTGAAG | | VYYCQQYDNWPPTWTFGQGTKVEIK | | | |
| | ATTTTGCAGTTTATTACTGTCAACAGTATGATAACTGGCCTCCCACGTGGACGTTCGGCCA | | | | | |
| | AGGGACCAAGGTGGAAATCAAA | | | | | |
| ADI-75718 | GAAATGACACTGACGCAGTCTCCAGCCACCCTGTCTGTGTCTCCAGGGGAGACAGTCACCC | 3159 | EMTLTQSPATLSVSPGETVTLSCRASQS | 3441 | | |
| | TCTCCTGCAGGGCCAGTCAGAGTCTCAGTAATAAGTTAGCCTGGTACCAACAGAGACCTGG | | LSNKLAWYQQRPGQAPTLLIHGASGRAT | | | |
| | CCAGGCTCCCACCCTCCTCATCCATGGTGCTTCCGGCAGGGCCACTGGTCTCCCAGACCGG | | GLPDRFSGSGYGTEFTLTIRRLQSEDFA | | | |
| | TTCAGTGGCAGTGGGTATGGGACAGAGTTCACTCTCACCATCCGCCGCCTGCAGTCTGAAG | | VYYCQQYDSWPRTFGQGTKVEIK | | | |
| | ACTTTGCAGTTTATTACTGTCAGCAGTATGATTCCTGGCCTCGGACGTTCGGCCAAGGGAC | | | | | |
| | CAAGGTGGAAATCAAA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75836 | IGLV1-40 | ------ | 2005 | LGAAPTS | 2204 | GTSSFQ | 2352 | GNNNRP | 2492 | GVPDRFSG | 2655 | QSYDS | 2867 | FGGGTQ | 2948 |
| | | - | | GQVMMYT | | PLAPKL | | S | | SKSGTSAS | | RLSGV | | LTVL | |
| | | QCLGPQ | | | | LIY | | | | LAVTGLQA | | V | | | |
| | | GRGSPS | | | | | | | | EDEADYYC | | | | | |
| | | PA | | | | | | | | | | | | | |
| ADI-75574 | IGLV1-36 | QSVLTQ | 1989 | SGSSSNI | 2205 | WYQQLP | 2353 | YDHLLP | 2493 | GVSDRFSG | 2656 | AAWDD | 2868 | FGGGTK | 2939 |
| | | PPSVSE | | GKNAVN | | AQAPKL | | S | | SRSGTSAS | | SLNGL | | LTVL | |
| | | APRQRV | | | | LIY | | | | LAISGLQS | | V | | | |
| | | TISC | | | | | | | | EDEADYYC | | | | | |
| ADI-75620 | IGLV1-36 | QHELTQ | 2006 | SGSSANI | 2206 | WYQQLP | 2301 | YDDLLP | 2433 | GVSDRFSG | 2578 | ASWDD | 2869 | FGGGTK | 2939 |
| | | PPSVSE | | GHNAVN | | GKAPKL | | S | | SKSGTSAS | | RLNGP | | LTVL | |
| | | APRQRV | | | | LIY | | | | LAISGLQS | | V | | | |
| | | TISC | | | | | | | | EDEADYYC | | | | | |
| ADI-75634 | IGKV4-1 | TIVMTQ | 2007 | RSSQSVL | 2207 | WYQQKP | 2290 | WASTRE | 2422 | GVPDRFSG | 2632 | QQYYS | 2832 | FGPGTK | 2942 |
| | | SPDSVA | | YSSNNKN | | GQPPKL | | S | | SGSGTHFT | | SPPT | | VEIK | |
| | | VSLGER | | YLT | | LIY | | | | LTISSLQA | | | | | |
| | | ATINC | | | | | | | | EDVAVYYC | | | | | |
| ADI-75629 | IGKV1D- | EIQMTQ | 1881 | RASQSIS | 2050 | WYQQKP | 2354 | AASSLQ | 2391 | GVPSRFSG | 2657 | QQGYS | 2870 | FGQGTK | 2937 |
| | 39 | SPSSLS | | SYLN | | GKAPKL | | S | | SGSGTDFT | | TPYT | | VEIK | |
| | | ASVGDR | | | | LIN | | | | LTISSLQP | | | | | |
| | | VTITC | | | | | | | | EDFATYHC | | | | | |
| ADI-75834 | IGLV3-21 | SYVLTQ | 1883 | GGNNIGS | 2208 | WYQQKP | 2283 | DNSDRP | 2494 | GIPARFSG | 2658 | HVWDS | 2871 | FGTGTQ | 2943 |
| | | PPSVSV | | KSVH | | GQAPVL | | S | | SNSGNTAT | | SSDLH | | LTVL | |
| | | APGQTA | | | | VVY | | | | LTISRVEV | | V | | | |
| | | RITC | | | | | | | | GDEADYHC | | | | | |
| ADI-75747 | IGLV1-40 | QSVVTQ | 1972 | TGSNSNI | 2143 | WYQQLP | 2314 | DNNNRP | 2476 | GVPDRFSG | 2622 | QSYDS | 2872 | FGGGTQ | 2948 |
| | | PPSVSG | | GAGYDVH | | GTAPKL | | S | | SKSDTSAS | | SLIGS | | LTVL | |
| | | APGQRV | | | | LIY | | | | LAITGLQA | | L | | | |
| | | TISC | | | | | | | | EDEADYYC | | | | | |
| ADI-75648 | IGLV1-40 | QSVVTQ | 1972 | AGSNSNI | 2209 | WYQQLP | 2355 | GNTNRP | 2484 | GVPDRFSG | 2622 | QSYDS | 2840 | FGGGTK | 2939 |
| | | PPSVSG | | GAGYDVH | | GTAPQL | | S | | SKSDTSAS | | SLSDS | | LTVL | |
| | | APGQRV | | | | LIY | | | | LAITGLQA | | V | | | |
| | | TISC | | | | | | | | EDEADYYC | | | | | |
| ADI-75862 | IGLV2-11 | QSVLTQ | 2008 | TGNSSDV | 2210 | WYQQHP | 2356 | DVTKRP | 2495 | GVPNRFSG | 2659 | CSYSG | 2873 | FGPGTQ | 2957 |
| | | PRSVSG | | GDYNYVS | | GTAPKL | | S | | SKSGNTAS | | TYIPL | | LTVL | |
| | | SPGQSV | | | | MIY | | | | LTISGLQA | | I | | | |
| | | TISC | | | | | | | | EDEADYSC | | | | | |
| ADI-75693 | IGLV3-21 | SYVLTQ | 1883 | GGNNIGS | 2208 | WYQQKP | 2283 | DDSDRP | 2399 | GIPERFSG | 2660 | QVWDG | 2874 | FGTGTK | 2945 |
| | | PPSVSV | | KSVH | | GQAPVL | | S | | SNSGNTAT | | SSDHF | | VTVL | |
| | | APGQTA | | | | VVY | | | | LTISRVEV | | YV | | | |
| | | RITC | | | | | | | | GDEADYYC | | | | | |
| ADI-75842 | IGLV2-8 | EPVLTQ | 2009 | TRTSNDV | 2211 | WYQLHP | 2357 | EVNKRP | 2394 | GVPDRFSG | 2651 | SSHTV | 2875 | FGAGTK | 2958 |
| | | PPSASG | | GNYNYVS | | GKAPKA | | S | | SKSGNTAS | | NNNFV | | LTVL | |
| | | SPGQSV | | | | LIY | | | | LTISGLQA | | | | | |
| | | TISC | | | | | | | | EDEADYYC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75836 | CAGTGTCTGGGGCCCCAGGGCAGAGGGTCACCATCTCCTGCACTGGGAGCAGCTCCAACAT | 3160 | QCLGPQGRGSPSPALGAAPTSGQVMMYT | 3442 | | |
| | CGGGGCAGGTTATGATGTACACTGGTACCAGCAGCTTCCAGCCCCTAGCCCCCAAACTCCT | | GTSSFQPLAPKLLIYGNNNRPSGVPDRF | | | |
| | CATCTATGGTAATAACAATCGGCCCTCAGGGGTCCCTGACCGATTCTCTGGCTCCAAGTCT | | SGSKSGTSASLAVTGLQAEDEADYYCQS | | | |
| | GGCACCTCAGCCTCCCTGGCCGTCACTGGGCTCCAGGCTGAGGATGAGGCTGATTATTACT | | YDSRLSGVVFGGGTQLTVL | | | |
| | GCCAGTCCTATGACAGCAGACTGAGTGGTGTGGTATTTGGCGGAGGGACCCAGCTCACCGT | | | | | |
| | CTTA | | | | | |
| ADI-75574 | CAGTCTGTGCTGACTCAGCCCCCCTCGGTGTCTGAAGCCCCCAGGCAGAGGGTCACCATCT | 3161 | QSVLTQPPSVSEAPRQRVTISCSGSSSN | 3443 | | |
| | CCTGTTCTGGAAGCAGCTCCAACATCGGAAAAAATGCTGTAAACTGGTACCAGCAGCTCCC | | IGKNAVNWYQQLPAQAPKLLIYYDHLLP | | | |
| | AGCACAGGCTCCCAAACTCCTCATCTACTATGATCATCTGTTGCCCTCAGGGGTCTCTGAC | | SGVSDRFSGSRSGTSASLAISGLQSEDE | | | |
| | CGATTCTCTGGCTCCAGGTCTGGCACCTCAGCCTCCCTGGCCATCAGTGGGCTCCAGTCTG | | ADYYCAAWDDSLNGLVFGGGTKLTVL | | | |
| | AGGATGAGGCTGATTATTACTGTGCAGCATGGGATGACAGCCTGAATGGCCTTGTATTCGG | | | | | |
| | CGGAGGGACCAAGCTCACCGTCCTA | | | | | |
| ADI-75620 | CAGCATGAGCTGACTCAGCCACCCTCGGTGTCTGAAGCCCCCAGGCAGAGGGTCACCATCT | 3162 | QHELTQPPSVSEAPRQRVTISCSGSSAN | 3444 | QSVLTQPPS | 3527 |
| | CCTGTTCTGGAAGCAGCGCCAACATCGGACATAATGCTGTGAACTGGTACCAGCAGCTCCC | | IGHNAVNWYQQLPGKAPKLLIYYDDLLP | | VSEAPRQRV | |
| | AGGAAAGGCTCCCAAGCTCCTCATCTATTATGATGATCTGCTGCCCTCAGGGGTCTCTGAC | | SGVSDRFSGSKSGTSASLAISGLQSEDE | | TISCSGSSA | |
| | CGATTCTCTGGCTCCAAGTCTGGCACCTCAGCCTCCCTGGCCATCAGTGGGCTCCAGTCTG | | ADYYCASWDDRLNGPVFGGGTKLTVL | | NIGHNAVNW | |
| | AGGATGAGGCTGATTATTACTGTGCATCATGGGATGACAGGCTGAATGGTCCGGTGTTCGG | | | | YQQLPGKAP | |
| | CGGAGGGACCAAGCTGACCGTCCTA | | | | KLLIYYDDL | |
| | | | | | LPSGVSDRF | |
| | | | | | SGSKSGTSA | |
| | | | | | SLAISGLQS | |
| | | | | | EDEADYYCA | |
| | | | | | SWDDRLNGP | |
| | | | | | VFGGGTKLT | |
| | | | | | VL | |
| ADI-75634 | ACAATCGTGATGACGCAGTCTCCAGATTCCGTGGCTGTGTCTCTGGGCGAGAGGGCCACCA | 3163 | TIVMTQSPDSVAVSLGERATINCRSSQS | 3445 | | |
| | TCAACTGCAGGTCCAGCCAGAGTGTTTTATACAGCTCCAACAATAAGAATTACTTAACTTG | | VLYSSNNKNYLTWYQQKPGQPPKLLIYW | | | |
| | GTACCAGCAAAAACCAGGACAGCCTCCTAAGTTACTCATTTACTGGGCATCTACCCGGGAA | | ASTRESGVPDRFSGSGSGTHFTLTISSL | | | |
| | TCCGGGGTCCCTGACCGATTCAGTGGCAGCGGGTCTGGGACGCATTTCACTCTCACCATCA | | QAEDVAVYYCQQYYSSPPTFGPGTKVEI | | | |
| | GCAGCCTGCAGGCTGAAGATGTGGCGGTTTATTATTGTCAGCAGTATTATAGTAGTCCTCC | | K | | | |
| | CACTTTCGGCCCTGGGACCAAGGTGGAGATCAAA | | | | | |
| ADI-75629 | GAAATCCAAATGACGCAGTCTCCATCCTCCCTGTCTGCATCTGTAGGAGACAGAGTCACCA | 3164 | EIQMTQSPSSLSASVGDRVTITCRASQS | 3446 | | |
| | TCACTTGCCGGGCAAGTCAGAGCATTAGCAGCTATTTAAATTGGTATCAGCAGAAACCAGG | | ISSYLNWYQQKPGKAPKLLINAASSLQS | | | |
| | GAAAGCCCCTAAGCTCCTGATTAATGCGGCATCCAGTTTGCAAAGTGGGGTCCCATCAAGG | | GVPSRFSGSGSGTDFTLTISSLQPEDFA | | | |
| | TTCAGTGGCAGTGGATCTGGGACAGATTTCACTCTCACCATCAGCAGTCTGCAACCTGAAG | | TYHCQQGYSTPYTFGQGTKVEIK | | | |
| | ATTTTGCAACTTACCACTGTCAACAGGGATACAGTACCCCGTACACTTTTGGCCAGGGGAC | | | | | |
| | CAAGGTGGAAATCAAA | | | | | |
| ADI-75834 | TCGTATGTGCTGACTCAGCCACCCTCGGTGTCAGTGGCCCCAGGACAGACGGCCAGGATTA | 3165 | SYVLTQPPSVSVAPGQTARITCGGNNIG | 3447 | | |
| | CGTGTGGGGGAAATAACATTGGAAGTAAAAGTGTGCACTGGTACCAGCAGAAGCCCGGCCA | | SKSVHWYQQKPGQAPVLVVYDNSDRPSG | | | |
| | GGCCCCTGTGCTGGTCGTCTATGATAATAGCGACCGGCCCTCAGGGATCCCTGCGCGATTC | | IPARFSGSNSGNTATLTISRVEVGDEAD | | | |
| | TCTGGCTCCAATTCTGGGAACACGGCCACCCTGACCATCAGCAGGGTCGAAGTCGGGGATG | | YHCHVWDSSSDLHVFGTGTQLTVL | | | |
| | AGGCCGACTATCACTGTCACGTGTGGGATAGTAGTAGTGATCTTCATGTCTTCGGAACTGG | | | | | |
| | GACCCAGCTCACCGTCCTA | | | | | |
| ADI-75747 | CAGTCTGTCGTGACGCAGCCGCCCTCAGTGTCTGGGGCCCCAGGGCAGAGGGTCACCATCT | 3166 | QSVVTQPPSVSGAPGQRVTISCTGSNSN | 3448 | | |
| | CCTGCACTGGGAGCAACTCCAATATCGGGGCAGGTTATGATGTACACTGGTACCAGCAGCT | | IGAGYDVHWYQQLPGTAPKLLIYDNNNR | | | |
| | TCCAGGAACAGCCCCCAAACTCCTCATCTATGATAATAACAATCGGCCCTCAGGGGTCCCT | | PSGVPDRFSGSKSDTSASLAITGLQAED | | | |
| | GACCGATTCTCTGGCTCCAAGTCTGACACCTCAGCCTCCCTGGCCATCACTGGGCTCCAGG | | EADYYCQSYDSSLIGSLFGGGTQLTVL | | | |
| | CTGAGGATGAGGCTGATTATTACTGCCAGTCCTATGACAGCAGCCTGATTGGTTCGCTGTT | | | | | |
| | CGGCGGAGGGACCCAGCTCACCGTCCTC | | | | | |
| ADI-75648 | CAGTCTGTGGTGACTCAGCCGCCCTCAGTGTCTGGGGCCCCAGGTCAGAGGGTCACCATCT | 3167 | QSVVTQPPSVSGAPGQRVTISCAGSNSN | 3449 | | |
| | CCTGCGCTGGGAGCAACTCCAACATCGGGGCAGGTTATGATGTTCACTGGTACCAGCAGCT | | IGAGYDVHWYQQLPGTAPQLLIYGNTNR | | | |
| | TCCAGGAACAGCCCCCCAACTCCTCATCTATGGTAACACCAATCGGCCCTCAGGGGTCCCT | | PSGVPDRFSGSKSDTSASLAITGLQAED | | | |
| | GACCGGTTCTCTGGCTCCAAGTCTGACACCTCAGCCTCCCTGGCCATCACTGGGCTCCAGG | | EADYYCQSYDSSLSDSVFGGGTKLTVL | | | |
| | CTGAGGATGAGGCCGATTATTACTGCCAGTCCTATGACAGCAGCCTGAGTGATTCGGTGTT | | | | | |
| | CGGCGGAGGGACCAAGCTCACCGTCCTA | | | | | |
| ADI-75862 | CAGTCTGTGCTGACTCAGCCTCGCTCAGTGTCCGGGTCTCCTGGACAGTCAGTCACCATCT | 3168 | QSVLTQPRSVSGSPGQSVTISCTGNSSD | 3450 | | |
| | CCTGCACTGGAAATAGCAGTGATGTTGGTGATTATAATTATGTCTCCTGGTACCAACAACA | | VGDYNYVSWYQQHPGTAPKLMIYDVTKR | | | |
| | CCCCGGCACAGCCCCCAAACTCATGATTTATGATGTCACTAAGCGGCCCTCAGGGGTCCCT | | PSGVPNRFSGSKSGNTASLTISGLQAED | | | |
| | AATCGCTTCTCTGGCTCCAAGTCTGGCAACACGGCCTCCCTGACCATCTCTGGGCTCCAGG | | EADYSCCSYSGTYIPLIFGPGTQLTVL | | | |
| | CTGAGGATGAGGCTGATTATTCCTGCTGTTCATATTCAGGAACCTACATTCCTTTAATCTT | | | | | |
| | CGGACCTGGCACCCAGCTGACCGTCCTC | | | | | |
| ADI-75693 | TCGTATGTGCTGACTCAGCCACCCTCGGTGTCAGTGGCCCCAGGACAGACGGCCAGGATTA | 3169 | SYVLTQPPSVSVAPGQTARITCGGNNIG | 3451 | | |
| | CCTGTGGGGGAAACAATATTGGAAGTAAAAGTGTGCACTGGTACCAGCAGAAGCCAGGCCA | | SKSVHWYQQKPGQAPVLVVYDDSDRPSG | | | |
| | GGCCCCTGTGCTGGTCGTCTATGATGATAGCGACCGGCCCTCAGGGATCCCTGAGCGATTC | | IPERFSGSNSGNTATLTISRVEVGDEAD | | | |
| | TCTGGCTCCAACTCTGGAAACACGGCCACACTGACCATCAGCAGGGTCGAAGTCGGGGATG | | YYCQVWDGSSDHFYVFGTGTKVTVL | | | |
| | AGGCCGACTATTACTGTCAGGTGTGGGATGGTAGTAGTGATCATTTTTATGTCTTCGGAAC | | | | | |
| | TGGGACCAAGGTCACCGTCCTA | | | | | |
| ADI-75842 | GAGCCTGTGTTGACTCAGCCTCCCTCCGCGTCCGGGTCTCCTGGACAGTCAGTCACCATCT | 3170 | EPVLTQPPSASGSPGQSVTISCTRTSND | 3452 | | |
| | CCTGCACTAGAACCAGCAATGACGTTGGTAATTATAACTATGTCTCCTGGTACCAGCTGCA | | VGNYNYVSWYQLHPGKAPKALIYEVNKR | | | |
| | CCCAGGCAAAGCCCCCAAAGCCTTGATTTATGAGGTCAATAAGCGGCCCTCAGGGGTCCCT | | PSGVPDRFSGSKSGNTASLTISGLQAED | | | |
| | GATCGCTTCTCTGGCTCCAAGTCTGGCAACACGGCCTCCCTGACCATCTCTGGGCTCCAGG | | EADYYCSSHTVNNNFVFGAGTKLTVL | | | |
| | CTGAGGATGAGGCTGATTATTACTGCAGCTCACATACAGTCAATAACAATTTTGTCTTCGG | | | | | |
| | AGCTGGGACCAAGCTCACCGTCCTA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75828 | IGLV3-21 | QSVLTQ | 2010 | GGNNIGS | 2208 | WYQQKP | 2358 | DDSDRP | 2399 | GIPERFSG | 2545 | QVWDS | 2876 | FGGGTK | 2939 |
| | | PPSVSV | | KSVH | | GQAPVL | | S | | SNSGNTAT | | SSDLH | | LTVL | |
| | | APGQTA | | | | VVN | | | | LTISRVEA | | W | | | |
| | | RITC | | | | | | | | GDEADYYC | | | | | |
| ADI-75632 | IGLV1-40 | SAVLTQ | 2011 | TGSSSNI | 2201 | WYQQLP | 2359 | GNSNRP | 2477 | GVPDRFSG | 2661 | QSYDS | 2877 | FGGGTK | 2939 |
| | | PPSVSG | | GAGYDVH | | RTAPKL | | S | | SKSGTSAS | | SASGS | | LTVL | |
| | | APGQRI | | | | LIY | | | | LAITGVQG | | V | | | |
| | | TISC | | | | | | | | EDEGDYYC | | | | | |
| ADI-75855 | IGKV1-33 | DIQMTQ | 1878 | QASQDIS | 2212 | WYQQKP | 2257 | DASNLE | 2416 | GVPSRFSG | 2662 | QQYDN | 2878 | FGQGTR | 2940 |
| | | SPSSLS | | NYLN | | GKAPKL | | T | | SGSGTEFT | | LPII | | LEIK | |
| | | ASVGDR | | | | LIY | | | | FTINSLQP | | | | | |
| | | VTITC | | | | | | | | EDIATYYC | | | | | |
| ADI-75729 | IGLV1-40 | QYVVTQ | 2012 | TESRSNI | 2213 | GYQQFP | 2360 | GNNIRP | 2496 | RVHDRLFG | 2663 | QSYDS | 2879 | FGIGTK | 2959 |
| | | PPSVFG | | GAGFDVQ | | GTAPKL | | S | | SKCDTSAS | | MLSDP | | VTVL | |
| | | ARGQRV | | | | IIY | | | | MAITGIQG | | YV | | | |
| | | TISC | | | | | | | | EDEAYYYC | | | | | |
| ADI-75734 | IGKV1D- | DIQLTQ | 1876 | RASHRID | 2098 | WYQQKP | 2291 | VASTLQ | 2423 | GVPSRFTG | 2551 | QQSYT | 2752 | FGPGTK | 2942 |
| | 39 | SPSSLS | | RYLN | | GQAPKL | | S | | SGSGTDFT | | TPRT | | VEIK | |
| | | ASVGDR | | | | LIY | | | | LTISSLQP | | | | | |
| | | VTITC | | | | | | | | EDFATYYC | | | | | |
| ADI-75880 | IGLV1-44 | SYELTQ | 1962 | SGSTSNI | 2214 | WYQHLP | 2307 | SDDLRP | 2497 | GVPDRFSG | 2664 | ATWDD | 2880 | FGGGTQ | 2948 |
| | | PPSASG | | GSNTVK | | GTAPKL | | S | | SKSGTSAS | | TLAGP | | LTVL | |
| | | TPGQRV | | | | LIY | | | | LAISALQS | | V | | | |
| | | TISC | | | | | | | | EDESHYYC | | | | | |
| ADI-75724 | IGLV3-21 | SYVLTQ | 2013 | GGNNIGT | 2215 | WYQQKP | 2304 | YDSDRP | 2498 | GTPERFSG | 2665 | QVWDS | 2881 | FGEGTK | 2960 |
| | | PPSVSV | | KSVH | | GQAPVL | | S | | SNSGNTAT | | NSDHR | | VTVL | |
| | | APGKTA | | | | VIY | | | | LTISRVEA | | V | | | |
| | | SLTC | | | | | | | | GDEADYYC | | | | | |
| ADI-75737 | IGLV1-40 | QSVVTQ | 2014 | TGSSSNI | 2201 | WYQQVP | 2361 | RNTHRP | 2499 | GVPDRFSG | 2666 | QSYDS | 2840 | FGGGTK | 2947 |
| | | PPSVSG | | GAGYDVH | | GKAPTL | | S | | SRSGTSAS | | SLSDS | | VTVL | |
| | | APGQRV | | | | LIY | | | | LAITGLQA | | V | | | |
| | | TIPC | | | | | | | | EDEADYYC | | | | | |
| ADI-75610 | IGLV3-21 | ETTLTQ | 2015 | EGDNIGR | 2216 | WYQQKP | 2362 | GDGDRP | 2500 | GIPERLSG | 2667 | QVWDS | 2882 | FGGGTQ | 2948 |
| | | SPSVSV | | KSVH | | AQAPVL | | S | | STSGNTAT | | SSDLV | | LTVL | |
| | | APGQTA | | | | VVY | | | | LTISRVEA | | L | | | |
| | | RITC | | | | | | | | GDEADYYC | | | | | |
| ADI-75603 | IGLV1-44 | SYELTQ | 1962 | SGSSSNI | 2217 | WYQQLP | 2314 | SNNQRP | 2463 | GIPDRFSG | 2668 | AAWDD | 2883 | FGGGTK | 2947 |
| | | PPSASG | | GSNTVN | | GTAPKL | | S | | SKSGTSAS | | SLNGR | | VTVL | |
| | | TPGQRV | | | | LIY | | | | LVISGLQS | | LL | | | |
| | | TISC | | | | | | | | EDEAGYYC | | | | | |
| ADI-75649 | IGLV1-47 | SYELTQ | 1962 | SGSSSNI | 2218 | WYQQLP | 2314 | GDIQRP | 2501 | GVPDRFSG | 2669 | ATRDD | 2884 | FGGGTQ | 2948 |
| | | PPSASG | | GSNYVC | | GTAPKL | | S | | SKSGTSAS | | GRWV | | LTVL | |
| | | TPGQRV | | | | LIY | | | | LAISGLRS | | | | | |
| | | TISC | | | | | | | | EDEADYYC | | | | | |
| ADI-75625 | IGKV1D- | DIQMTQ | 1878 | RASQSTS | 2219 | WYQQKP | 2257 | AASTLQ | 2392 | GVPSRFSG | 2534 | QQSYT | 2885 | FGQGTK | 2937 |
| | 39 | SPSSLS | | IYLN | | GKAPKL | | S | | SGSGTDFT | | TRWT | | VEIK | |
| | | ASVGDR | | | | LIY | | | | LTISSLQP | | | | | |
| | | VTITC | | | | | | | | EDFATYYC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75828 | CAGTCTGTGCTGACTCAGCCACCCTCGGTGTCAGTGGCCCCAGGACAGACGGCCAGAATTA | 3171 | SKSVHWYQQKPGQAPVLVVNDDSDRPSG | 3453 | | |
| | CCTGTGGGGGAAACAACATTGGAAGTAAAAGTGTGCACTGGTACCAGCAGAAGCCAGGCCA | | IPERFSGSNSGNTATLTISRVEAGDEAD | | | |
| | GGCCCCTGTGCTGGTCGTCAATGATGATAGCGACCGGCCCTCAGGGATCCCTGAGCGATTC | | YYCQVWDSSSDLHVVFGGGTKLTVL | | | |
| | TCTGGCTCCAACTCTGGGAACACGGCCACCCTGACCATCAGCAGGGTCGAAGCCGGGGATG | | QSVLTQPPSVSVAPGQTARITCGGNNIG | | | |
| | AGGCCGACTATTACTGTCAGGTGTGGGATAGTAGTAGTGATCTTCATGTGGTATTCGGCGG | | | | | |
| | AGGGACCAAGCTCACCGTCCTA | | | | | |
| ADI-75632 | TCAGCTGTGCTGACTCAGCCACCCTCAGTGTCTGGGGCCCCAGGGCAGAGGATCACCATCT | 3172 | SAVLTQPPSVSGAPGQRITISCTGSSSN | 3454 | | |
| | CCTGCACTGGGAGCAGCTCCAACATCGGGGCAGGTTATGATGTACACTGGTACCAGCAGCT | | IGAGYDVHWYQQLPRTAPKLLIYGNSNR | | | |
| | TCCACGAACAGCCCCCAAACTCCTCATATATGGTAACAGCAATCGGCCCTCAGGGGTCCCT | | PSGVPDRFSGSKSGTSASLAITGVQGED | | | |
| | GACAGATTCTCTGGGTCCAAGTCTGGCACATCAGCCTCCCTGGCCATCACTGGGGTCCAGG | | EGDYYCQSYDSSASGSVFGGGTKLTVL | | | |
| | GTGAGGATGAGGGTGATTATTACTGCCAGTCGTATGACAGCAGCGCGAGTGGTTCGGTGTT | | | | | |
| | CGGCGGAGGGACCAAGCTCACCGTCCTA | | | | | |
| ADI-75855 | GACATCCAGATGACCCAGTCTCCATCCTCCCTGTCTGCATCTGTAGGAGACAGAGTCACCA | 3173 | DIQMTQSPSSLSASVGDRVTITCQASQD | 3455 | | |
| | TCACTTGCCAGGCGAGTCAGGACATTAGCAACTATTTAAATTGGTATCAGCAGAAACCAGG | | ISNYLNWYQQKPGKAPKLLIYDASNLET | | | |
| | GAAAGCCCCTAAGCTCCTGATCTACGATGCATCCAATTTGGAAACAGGGGTCCCATCAAGG | | GVPSRFSGSGSGTEFTFTINSLQPEDIA | | | |
| | TTCAGTGGAAGTGGATCTGGGACAGAATTTACTTTCACCATCAACAGCCTGCAGCCTGAAG | | TYYCQQYDNLPIIFGQGTRLEIK | | | |
| | ATATTGCAACATATTACTGTCAACAGTATGATAATCTCCCGATCATCTTCGGCCAAGGGAC | | | | | |
| | ACGACTGGAGATTAAA | | | | | |
| ADI-75729 | CAGTATGTGGTGACGCAGCCGCCTTCAGTGTTTGGGGCCAGAGGGCAGAGGGTCACCATCT | 3174 | QYVVTQPPSVFGARGQRVTISCTESRSN | 3456 | | |
| | CATGCACTGAGAGCAGATCCAACATCGGGGCAGGTTTTGATGTACAAGGGTACCAGCAATT | | IGAGFDVQGYQQFPGTAPKLIIYGNNIR | | | |
| | TCCAGGCACAGCCCCCAAACTCATCATCTATGGTAACAACATTCGGCCGTCACGGGTCCAT | | PSRVHDRLFGSKCDTSASMAITGIQGED | | | |
| | GACCGATTGTTTGGCTCCAAGTGTGACACCTCAGCCTCCATGGCCATCACTGGGATCCAGG | | EAYYYCQSYDSMLSDPYVFGIGTKVTVL | | | |
| | GTGAGGATGAGGCTTATTATTACTGCCAGTCCTATGACAGTATGCTGAGTGACCCTTATGT | | | | | |
| | TTTCGGAATAGGGACCAAGGTAACCGTCTTA | | | | | |
| ADI-75734 | GACATCCAGTTGACCCAGTCTCCATCCTCCCTGTCTGCATCTGTAGGAGACAGAGTCACCA | 3175 | DIQLTQSPSSLSASVGDRVTITCRASHR | 3457 | | |
| | TCACTTGTCGGGCAAGTCACAGGATTGACAGGTATTTAAATTGGTATCAGCAGAAACCAGG | | IDRYLNWYQQKPGQAPKLLIYVASTLQS | | | |
| | GCAAGCCCCCAAGCTCCTGATCTATGTAGCATCCACTTTGCAAAGTGGGGTCCCATCAAGG | | GVPSRFTGSGSGTDFTLTISSLQPEDFA | | | |
| | TTCACTGGCAGTGGATCTGGGACAGATTTCACTCTCACTATCAGCAGTCTGCAACCTGAGG | | TYYCQQSYTTPRTFGPGTKVEIK | | | |
| | ATTTTGCAACTTACTACTGTCAACAGAGTTACACTACCCCTCGGACTTTCGGCCCTGGGAC | | | | | |
| | CAAGGTGGAAATCAAA | | | | | |
| ADI-75880 | TCGTATGAGCTGACTCAGCCACCCTCAGCGTCTGGGACCCCCGGGCAGAGGGTCACCATCT | 3176 | SYELTQPPSASGTPGQRVTISCSGSTSN | 3458 | | |
| | CTTGTTCTGGAAGCACCTCCAACATCGGGAGTAATACTGTAAAATGGTACCAGCACCTCCC | | IGSNTVKWYQHLPGTAPKLLIYSDDLRP | | | |
| | AGGAACGGCCCCCAAACTCCTCATCTATAGTGATGATCTGCGACCCTCAGGGGTCCCTGAC | | SGVPDRFSGSKSGTSASLAISALQSEDE | | | |
| | CGATTCTCTGGCTCCAAGTCTGGCACCTCAGCCTCCCTGGCCATCAGTGCGCTCCAGTCTG | | SHYYCATWDDTLAGPVFGGGTQLTVL | | | |
| | AGGATGAGTCTCATTATTACTGTGCAACGTGGGATGACACTTTGGCTGGTCCGGTGTTCGG | | | | | |
| | CGGAGGCACCCAGCTCACCGTCCTA | | | | | |
| ADI-75724 | TCGTATGTGCTGACTCAGCCCCCCTCAGTGTCAGTGGCCCCAGGAAAGACGGCCAGCCTTA | 3177 | TKSVHWYQQKPGQAPVLVIYYDSDRPSG | 3459 | | |
| | CCTGTGGGGGAAACAACATTGGAACTAAGAGTGTGCACTGGTACCAGCAGAAGCCAGGCCA | | TPERFSGSNSGNTATLTISRVEAGDEAD | | | |
| | GGCCCCTGTGCTGGTCATCTATTATGATAGCGACCGGCCCTCAGGGACCCCTGAGCGATTC | | YYCQVWDSNSDHRVFGEGTKVTVL | | | |
| | TCTGGCTCCAACTCTGGGAACACGGCCACCCTGACCATCAGCAGGGTCGAAGCCGGGGATG | | SYVLTQPPSVSVAPGKTASLTCGGNNIG | | | |
| | AGGCCGACTATTACTGTCAGGTGTGGGATAGTAATAGTGATCATCGGGTCTTCGGCGAAGG | | | | | |
| | GACCAAGGTGACCGTCCTA | | | | | |
| ADI-75737 | CAGTCTGTCGTGACGCAGCCGCCCTCAGTGTCTGGGGCCCCAGGGCAGAGGGTCACCATCC | 3178 | QSVVTQPPSVSGAPGQRVTIPCTGSSSN | 3460 | | |
| | CCTGCACTGGGAGCAGCTCCAACATCGGGGCAGGTTATGATGTTCACTGGTACCAGCAGGT | | IGAGYDVHWYQQVPGKAPTLLIYRNTHR | | | |
| | CCCGGGAAAAGCCCCCACACTCCTCATCTATCGTAACACTCATCGGCCCTCAGGGGTCCCT | | PSGVPDRFSGSRSGTSASLAITGLQAED | | | |
| | GACCGATTCTCTGGCTCCAGGTCTGGCACCTCAGCCTCCCTGGCCATCACTGGACTCCAGG | | EADYYCQSYDSSLSDSVFGGGTKVTVL | | | |
| | CTGAGGATGAGGCTGATTATTACTGCCAGTCCTATGACAGCAGCCTGAGTGATTCGGTGTT | | | | | |
| | CGGCGGAGGGACCAAGGTCACCGTCCTA | | | | | |
| ADI-75610 | GAAACGACACTCACGCAGTCTCCTTCGGTGTCAGTGGCCCCAGGACAGACGGCCAGGATTA | 3179 | ETTLTQSPSVSVAPGQTARITCEGDNIG | 3461 | | |
| | CCTGTGAGGGAGACAACATTGGAAGGAAAAGTGTGCACTGGTACCAGCAGAAGCCAGCCCA | | RKSVHWYQQKPAQAPVLVVYGDGDRPSG | | | |
| | GGCCCCTGTGCTTGTCGTCTATGGTGATGGCGACCGGCCCTCAGGGATCCCTGAGCGACTC | | IPERLSGSTSGNTATLTISRVEAGDEAD | | | |
| | TCTGGCTCCACCTCTGGGAACACGGCCACCCTGACCATCAGCAGGGTCGAAGCCGGGGATG | | YYCQVWDSSSDLVLFGGGTQLTVL | | | |
| | AGGCCGACTATTACTGTCAGGTGTGGGACAGTAGTAGTGATCTCGTGCTGTTCGGCGGAGG | | | | | |
| | CACCCAGCTGACCGTCCTC | | | | | |
| ADI-75603 | TCCTATGAGCTGACACAGCCACCCTCGGCGTCTGGGACCCCCGGGCAGAGGGTCACCATCT | 3180 | SYELTQPPSASGTPGQRVTISCSGSSSN | 3462 | | |
| | CTTGTTCTGGAAGCAGCTCCAACATCGGAAGTAATACTGTAAACTGGTACCAGCAACTCCC | | IGSNTVNWYQQLPGTAPKLLIYSNNQRP | | | |
| | AGGAACGGCCCCCAAACTCCTCATCTATAGTAATAATCAGCGGCCCTCAGGGATCCCTGAC | | SGIPDRFSGSKSGTSASLVISGLQSEDE | | | |
| | CGATTCTCTGGCTCCAAGTCTGGCACCTCCGCCTCCCTGGTCATCAGTGGGCTCCAGTCTG | | AGYYCAAWDDSLNGRLLFGGGTKVTVL | | | |
| | AGGATGAGGCTGGTTATTACTGTGCAGCATGGGATGACAGCCTGAATGGTCGTCTACTATT | | | | | |
| | CGGCGGAGGGACCAAGGTGACCGTCCTA | | | | | |
| ADI-75649 | TCCTATGAGCTGACTCAGCCACCCTCAGCGTCTGGGACCCCCGGGCAGAGGGTCACCATCT | 3181 | SYELTQPPSASGTPGQRVTISCSGSSSN | 3463 | | |
| | CTTGTTCTGGAAGCAGCTCCAACATCGGAAGTAATTATGTATGCTGGTACCAGCAGCTCCC | | IGSNYVCWYQQLPGTAPKLLIYGDIQRP | | | |
| | AGGAACGGCCCCCAAACTCCTCATCTATGGTGATATTCAGCGGCCCTCAGGGGTCCCTGAC | | SGVPDRFSGSKSGTSASLAISGLRSEDE | | | |
| | CGATTCTCTGGCTCCAAGTCTGGCACCTCAGCCTCCCTGGCCATCAGTGGGCTCCGGTCCG | | ADYYCATRDDGRWVFGGGTQLTVL | | | |
| | AAGATGAGGCTGATTATTACTGTGCAACACGGGATGACGGCCGTTGGGTGTTCGGCGGAGG | | | | | |
| | GACCCAGCTGACCGTCCTA | | | | | |
| ADI-75625 | GACATCCAGATGACCCAGTCTCCATCCTCCCTGTCTGCATCTGTGGGAGACAGAGTCACCA | 3182 | DIQMTQSPSSLSASVGDRVTITCRASQS | 3464 | | |
| | TCACTTGCCGGGCAAGTCAGAGCACTAGCATCTATTTAAATTGGTATCAGCAGAAACCAGG | | TSIYLNWYQQKPGKAPKLLIYAASTLQS | | | |
| | GAAAGCCCCTAAGCTCCTAATCTATGCTGCATCCACTTTACAAAGTGGGGTCCCATCAAGG | | GVPSRFSGSGSGTDFTLTISSLQPEDFA | | | |
| | TTCAGTGGCAGTGGATCTGGGACAGATTTCACTCTCACCATCAGCAGTCTGCAACCTGAAG | | TYYCQQSYTTRWTFGQGTKVEIK | | | |
| | ATTTTGCAACTTACTACTGTCAACAGAGTTACACTACCCGGTGGACGTTCGGCCAAGGGAC | | | | | |
| | CAAGGTGGAAATCAAA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75589 | IGLV6-57 | KFMLTQ | 2016 | TGSSGSI | 2220 | WYQQRP | 2363 | EDDQRP | 2502 | GVPDRFSG | 2574 | QSYHS | 2886 | FGGGTK | 2939 |
| | | PHSVSE | | ANNYVQ | | GRAPTT | | S | | SIDSSSNS | | RNLWV | | LTVL | |
| | | APGKTV | | | | IIY | | | | ASLTISGL | | | | | |
| | | TISC | | | | | | | | KTEDEADY | | | | | |
| | | | | | | | | | | YC | | | | | |
| ADI-75763 | IGLV3-21 | QSVLTQ | 2017 | GGNSIGS | 2221 | WYQQKP | 2364 | DARDRP | 2503 | GIPERFSG | 2670 | QVWDS | 2887 | FGTGTK | 2949 |
| | | PPSVSV | | KSVH | | GQAPVL | | S | | SNSVNTAT | | SSDHY | | LTVL | |
| | | APGQTA | | | | VVS | | | | LTISRVEA | | V | | | |
| | | RIAC | | | | | | | | GDEADYYC | | | | | |
| ADI-75582 | IGLV3-1 | QPVLTQ | 2018 | SGDKLGD | 2222 | WYQKKP | 2365 | QDSERP | 2504 | GIPERFSG | 2671 | QAWDS | 2888 | FGGGTK | 2947 |
| | | PPSVSV | | KYAC | | GQSPVL | | S | | SNSGNTAT | | STAHW | | VTVL | |
| | | SPGQTA | | | | VIY | | | | LTISGTQA | | V | | | |
| | | TITC | | | | | | | | MDEADYYC | | | | | |
| ADI-75746 | IGLV6-57 | NFMLTQ | 1992 | TRSSGSI | 2109 | WYQQRP | 2366 | EDNERP | 2505 | GVPDRFSG | 2672 | QSYAS | 2889 | FGGGTK | 2939 |
| | | PHSVSE | | ASNYVQ | | GSAPTT | | S | | SIDRSSNS | | SPFYW | | LTVL | |
| | | SPGKTV | | | | VIF | | | | ASLTISAL | | V | | | |
| | | TISC | | | | | | | | KTEDEADY | | | | | |
| | | | | | | | | | | YC | | | | | |
| ADI-75726 | IGKV1D- | DIQVTQ | 2019 | RASQNIV | 2223 | WYQQKP | 2367 | AASSLQ | 2391 | GVPSRFSG | 2534 | QQSYS | 2729 | FGQGTK | 2944 |
| | 39 | SPSSLS | | SYLN | | GKAPKL | | S | | SGSGTDFT | | TPRT | | VDIK | |
| | | ASVGDR | | | | LMF | | | | LTISSLQP | | | | | |
| | | VTITC | | | | | | | | EDFATYYC | | | | | |
| ADI-75851 | IGLV3-21 | SYELTQ | 1919 | GGNNIGS | 2208 | WYQQKP | 2283 | DDSDRP | 2399 | GIPERFSG | 2673 | QVWDS | 2890 | FGTGTK | 2949 |
| | | PPSVSV | | KSVH | | GQAPVL | | S | | SNSGNTAT | | SSVHY | | LTVL | |
| | | APGQTA | | | | VVY | | | | LTISRVEV | | V | | | |
| | | RITC | | | | | | | | GDEADYFC | | | | | |
| ADI-75853 | IGKV1D- | DIEMTQ | 2020 | RASQSIS | 2134 | WYQQKP | 2257 | AASSLQ | 2391 | GVPSRFSG | 2534 | QQSYR | 2891 | FGPGTK | 2942 |
| | 39 | SPSSLS | | RYLN | | GKAPKL | | S | | SGSGTDFT | | TPPLT | | VEIK | |
| | | ASVGDR | | | | LIY | | | | LTISSLQP | | | | | |
| | | VTISC | | | | | | | | EDFATYYC | | | | | |
| ADI-75863 | IGLV3-21 | SYVLTQ | 2021 | GGNNIGS | 2208 | WYQQKP | 2283 | DDSDRP | 2399 | GIPERFSG | 2674 | QVWDS | 2892 | FGGGTK | 2939 |
| | | PPSVSV | | KSVH | | GQAPVL | | S | | SSSGNTAT | | SSDLV | | LTVL | |
| | | APGQTA | | | | WVY | | | | LTLSRVEA | | I | | | |
| | | RIPC | | | | | | | | GDEADYYC | | | | | |
| ADI-75753 | IGLV1-40 | QSVLTQ | 1948 | T- | 2224 | WYQQFP | 2368 | GNNIRP | 2496 | GVPDRFSG | 2599 | QSSDT | 2893 | FGGGTQ | 2948 |
| | | PPSVSG | | ASSNIGA | | GRAPKL | | S | | SKSGTSAS | | SLSDW | | LTVL | |
| | | APGQRV | | GYDVH | | LIY | | | | LAITGLQA | | V | | | |
| | | TISC | | | | | | | | EDEADYYC | | | | | |
| ADI-75756 | IGKV1D- | ASVGDR | 1881 | RASQSIS | 2225 | WYQQKP | 2257 | TASSLQ | 2506 | GVPSRFSG | 2606 | QQSYE | 2894 | FGQGTK | 2937 |
| | 39 | EIQMTQ | | RHLN | | GKAPKL | | S | | SGSGTDFT | | TPPWT | | VEIK | |
| | | SPSSLS | | | | LIY | | | | LTISSLQP | | | | | |
| | | VTITC | | | | | | | | EDFASYYC | | | | | |
| ADI-75769 | IGKV1-5 | DIQMTQ | 1879 | RASQSIG | 2226 | WYQQKP | 2369 | DASSLE | 2507 | GVPSRFSG | 2675 | QQYNG | 2895 | FGGGTK | 2936 |
| | | SPSTLS | | SWLA | | GKAPKL | | T | | SGSGTEFT | | YSPGL | | VEIK | |
| | | ASVGDR | | | | VIY | | | | LTISSLQP | | T | | | |
| | | VTITC | | | | | | | | DDFASYYC | | | | | |
| ADI-75772 | IGKV1D- | DIQMTQ | 1878 | RASQSIS | 2050 | WYQQKP | 2257 | AASVLQ | 2508 | GVPSRFSG | 2676 | QQSYS | 2896 | FGQGTK | 2937 |
| | 39 | SPSSLS | | SYLN | | GKAPKL | | S | | SGSETDFT | | NFRT | | VEIK | |
| | | ASVGDR | | | | LIY | | | | LTISSLQP | | | | | |
| | | VTITC | | | | | | | | EDFATYYC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75589 | AAGTTTATGCTGACTCAGCCCCACTCTGTGTCGGAGGCTCCGGGGAAGACGGTAACCATCT | 3183 | KFMLTQPHSVSEAPGKTVTISCTGSSGS | 3465 | | |
| | CCTGCACCGGCAGCAGTGGCAGCATTGCCAACAACTATGTGCAGTGGTATCAGCAGCGCCC | | IANNYVQWYQQRPGRAPTTIIYEDDQRP | | | |
| | GGGCAGAGCCCCCACCACTATTATCTATGAGGATGACCAAAGACCCTCTGGGGTCCCTGAT | | SGVPDRFSGSIDSSSNSASLTISGLKTE | | | |
| | CGCTTCTCTGGCTCCATCGACAGTTCCTCCAACTCTGCCTCCCTCACCATCTCCGGGCTGA | | DEADYYCQSYHSRNLWVFGGGTKLTVL | | | |
| | AGACTGAGGACGAGGCTGACTACTACTGTCAGTCTTATCATAGCCGCAATCTTTGGGTGTT | | | | | |
| | CGGCGGAGGCACCAAGCTCACCGTCCTA | | | | | |
| ADI-75763 | CAGTCTGTGCTGACTCAGCCACCCTCGGTGTCAGTGGCCCCTGGACAGACGGCCAGGATTG | 3184 | QSVLTQPPSVSVAPGQTARIACGGNSIG | 3466 | | |
| | CCTGTGGGGGAAACAGCATTGGAAGTAAAAGTGTGCACTGGTACCAGCAGAAGCCAGGCCA | | SKSVHWYQQKPGQAPVLVVSDARDRPSG | | | |
| | GGCCCCTGTGCTGGTCGTCTCTGATGCTCGCGACCGGCCCTCAGGGATCCCTGAGCGATTC | | IPERFSGSNSVNTATLTISRVEAGDEAD | | | |
| | TCTGGCTCCAACTCTGTGAACACGGCCACCCTGACCATCAGCAGGGTCGAAGCCGGGGATG | | YYCQVWDSSSDHYVFGTGTKLTVL | | | |
| | AGGCCGACTATTATTGTCAGGTGTGGGATAGTAGTAGTGACCATTATGTCTTCGGAACTGG | | | | | |
| | GACCAAGCTCACCGTCCTA | | | | | |
| ADI-75582 | CAGCCTGTGCTGACTCAGCCACCCTCAGTGTCCGTGTCCCCAGGACAGACAGCCACCATCA | 3185 | QPVLTQPPSVSVSPGQTATITCSGDKLG | 3467 | | |
| | CCTGCTCTGGAGATAAATTGGGGGATAAATATGCTTGCTGGTATCAGAAGAAGCCAGGCCA | | DKYACWYQKKPGQSPVLVIYQDSERPSG | | | |
| | GTCCCCTGTATTGGTCATCTATCAAGATTCCGAGCGGCCCTCAGGGATCCCTGAGCGATTC | | IPERFSGSNSGNTATLTISGTQAMDEAD | | | |
| | TCTGGCTCCAACTCTGGGAACACAGCCACTCTGACCATCAGCGGGACCCAGGCTATGGATG | | YYCQAWDSSTAHWVFGGGTKVTVL | | | |
| | AGGCTGACTATTACTGTCAGGCGTGGGACAGCAGCACTGCCCATTGGGTGTTCGGCGGAGG | | | | | |
| | GACCAAGGTGACCGTCCTA | | | | | |
| ADI-75746 | AATTTTATGCTGACTCAGCCCCACTCTGTGTCGGAGTCTCCGGGGAAGACAGTAACCATCT | 3186 | NFMLTQPHSVSESPGKTVTISCTRSSGS | 3468 | | |
| | CCTGCACCCGCAGCAGTGGCAGCATTGCCAGCAACTATGTGCAGTGGTACCAGCAGCGCCC | | IASNYVQWYQQRPGSAPTTVIFEDNERP | | | |
| | GGGGAGTGCCCCCACCACTGTGATCTTTGAGGATAATGAGAGACCCTCTGGGGTCCCTGAT | | SGVPDRFSGSIDRSSNSASLTISALKTE | | | |
| | CGGTTCTCTGGCTCCATCGACAGGTCCTCCAACTCTGCCTCCCTCACCATCTCTGCACTGA | | DEADYYCQSYASSPFYWVFGGGTKLTVL | | | |
| | AGACTGAGGACGAGGCTGACTACTATTGTCAGTCTTATGCTAGCAGCCCCTTCTATTGGGT | | | | | |
| | GTTTGGCGGAGGGACCAAGCTGACCGTCCTA | | | | | |
| ADI-75726 | GACATCCAGGTGACCCAGTCTCCATCCTCCCTGTCTGCATCTGTAGGAGACAGAGTCACCA | 3187 | DIQVTQSPSSLSASVGDRVTITCRASQN | 3469 | | |
| | TCACTTGCCGGGCAAGTCAGAACATTGTCAGCTATTTAAATTGGTATCAGCAGAAACCAGG | | IVSYLNWYQQKPGKAPKLLMFAASSLQS | | | |
| | GAAAGCCCCTAAGCTCCTGATGTTTGCTGCATCCAGTTTGCAAAGTGGGGTCCCATCAAGG | | GVPSRFSGSGSGTDFTLTISSLQPEDFA | | | |
| | TTCAGTGGCAGTGGATCTGGGACAGATTTCACTCTCACCATCAGCAGTCTGCAACCTGAAG | | TYYCQQSYSTPRTFGQGTKVDIK | | | |
| | ACTTTGCAACTTACTACTGTCAACAGAGTTATAGTACCCCTCGAACGTTCGGCCAAGGGAC | | | | | |
| | CAAAGTGGATATCAAA | | | | | |
| ADI-75851 | TCGTATGAGCTGACTCAGCCACCCTCGGTGTCAGTGGCCCCAGGACAGACGGCCAGGATTA | 3188 | SYELTQPPSVSVAPGQTARITCGGNNIG | 3470 | | |
| | CCTGTGGGGGAAACAACATTGGAAGTAAAAGTGTGCACTGGTACCAGCAGAAGCCAGGCCA | | SKSVHWYQQKPGQAPVLVVYDDSDRPSG | | | |
| | GGCCCCTGTACTGGTCGTCTATGATGATAGCGACCGGCCCTCAGGGATCCCTGAGCGATTC | | IPERFSGSNSGNTATLTISRVEVGDEAD | | | |
| | TCTGGCTCCAACTCTGGGAACACGGCCACCCTGACCATCAGCAGGGTCGAAGTCGGGGATG | | YFCQVWDSSSVHYVFGTGTKLTVL | | | |
| | AGGCCGACTATTTCTGTCAGGTGTGGGATAGTAGTAGTGTTCATTATGTCTTCGGAACTGG | | | | | |
| | GACCAAGCTCACCGTCCTA | | | | | |
| ADI-75853 | GACATCGAGATGACCCAGTCTCCATCCTCCCTGTCTGCATCTGTAGGAGACAGAGTCACCA | 3189 | DIEMTQSPSSLSASVGDRVTISCRASQS | 3471 | | |
| | TCTCCTGCCGGGCAAGTCAGAGCATTAGCAGGTATTTAAATTGGTATCAGCAGAAACCAGG | | ISRYLNWYQQKPGKAPKLLIYAASSLQS | | | |
| | GAAAGCCCCTAAGCTCCTGATCTATGCTGCATCCAGTTTGCAAAGTGGGGTCCCATCAAGG | | GVPSRFSGSGSGTDFTLTISSLQPEDFA | | | |
| | TTCAGTGGCAGTGGATCTGGGACAGATTTCACTCTCACCATCAGCAGTCTGCAACCTGAAG | | TYYCQQSYRTPPLTFGPGTKVEIK | | | |
| | ATTTTGCAACTTACTACTGTCAACAGAGTTACAGAACCCCCCCCCTCACTTTCGGCCCTGG | | | | | |
| | GACCAAGGTGGAAATCAAA | | | | | |
| ADI-75863 | TCGTATGTGCTGACTCAGCCACCCTCGGTGTCAGTGGCCCCAGGACAGACGGCCAGGATTC | 3190 | SYVLTQPPSVSVAPGQTARIPCGGNNIG | 3472 | | |
| | CCTGTGGGGGAAACAACATTGGAAGTAAAAGTGTGCACTGGTACCAGCAGAAGCCAGGCCA | | SKSVHWYQQKPGQAPVLVVYDDSDRPSG | | | |
| | GGCCCCTGTGCTGGTCGTCTATGATGATAGCGACCGGCCCTCAGGGATCCCTGAGCGATTC | | IPERFSGSSSGNTATLTLSRVEAGDEAD | | | |
| | TCTGGCTCCAGCTCTGGGAACACGGCCACCCTGACCCTCAGCAGGGTCGAAGCCGGGGATG | | YYCQVWDSSSDLVIFGGGTKLTVL | | | |
| | AGGCCGACTATTACTGTCAAGTGTGGGATAGTAGTAGTGATCTTGTGATATTCGGCGGAGG | | | | | |
| | GACCAAGCTCACCGTCCTA | | | | | |
| ADI-75753 | CAGTCTGTGTTGACGCAGCCGCCCTCAGTGTCTGGGGCCCCAGGGCAGAGGGTCACCATCT | 3191 | QSVLTQPPSVSGAPGQRVTISCTASSNI | 3473 | | |
| | CCTGCACTGCGAGCAGCAACATCGGGGCAGGTTATGATGTACACTGGTATCAGCAGTTTCC | | GAGYDVHWYQQFPGRAPKLLIYGNNIRP | | | |
| | AGGAAGAGCCCCCAAACTCCTCATCTATGGTAACAACATTCGGCCCTCAGGGGTCCCTGAC | | SGVPDRFSGSKSGTSASLAITGLQAEDE | | | |
| | CGATTCTCTGGTTCCAAGTCTGGCACCTCAGCCTCCCTGGCCATCACTGGGCTCCAGGCTG | | ADYYCQSSDTSLSDWVFGGGTQLTVL | | | |
| | AGGATGAGGCTGATTATTACTGCCAGTCGTCTGACACCAGCCTGAGTGATTGGGTGTTCGG | | | | | |
| | CGGAGGCACCCAGCTCACCGTCCTC | | | | | |
| ADI-75756 | GAAATCCAGATGACCCAGTCTCCATCCTCCCTGTCTGCATCTGTAGGAGACAGAGTCACCA | 3192 | EIQMTQSPSSLSASVGDRVTITCRASQS | 3474 | | |
| | TCACTTGCCGGGCAAGTCAGAGCATTAGCAGGCACTTAAATTGGTATCAGCAGAAACCAGG | | ISRHLNWYQQKPGKAPKLLIYTASSLQS | | | |
| | GAAAGCCCCTAAGCTCCTGATCTATACTGCATCCAGTTTGCAAAGTGGGGTCCCATCAAGG | | GVPSRFSGSGSGTDFTLTISSLQPEDFA | | | |
| | TTCAGTGGCAGTGGATCTGGGACAGATTTCACTCTCACCATCAGCAGTCTGCAACCTGAAG | | SYYCQQSYETPPWTFGQGTKVEIK | | | |
| | ATTTTGCAAGTTACTACTGTCAACAGAGTTACGAAACCCCTCCGTGGACGTTCGGCCAAGG | | | | | |
| | GACCAAGGTGGAAATCAAA | | | | | |
| ADI-75769 | GACATCCAGATGACCCAGTCTCCTTCCACCCTGTCTGCATCTGTAGGAGACAGAGTCACCA | 3193 | DIQMTQSPSTLSASVGDRVTITCRASQS | 3475 | | |
| | TCACTTGCCGGGCCAGTCAGAGTATCGGTAGCTGGTTGGCCTGGTATCAGCAGAAACCAGG | | IGSWLAWYQQKPGKAPKLVIYDASSLET | | | |
| | GAAAGCCCCTAAGTTAGTGATCTATGATGCCTCCAGTTTGGAAACTGGGGTCCCATCAAGG | | GVPSRFSGSGSGTEFTLTISSLQPDDFA | | | |
| | TTCAGCGGCAGTGGATCTGGGACAGAATTCACTCTCACCATCAGCAGCCTGCAGCCTGATG | | SYYCQQYNGYSPGLTFGGGTKVEIK | | | |
| | ATTTTGCATCTTATTACTGCCAACAGTATAATGGTTATTCTCCGGGGCTCACTTTCGGCGG | | | | | |
| | AGGGACCAAGGTGGAAATCAAA | | | | | |
| ADI-75772 | GACATCCAGATGACCCAGTCTCCATCCTCCCTGTCTGCATCTGTCGGAGACAGAGTCACCA | 3194 | DIQMTQSPSSLSASVGDRVTITCRASQS | 3476 | | |
| | TCACTTGCCGGGCAAGTCAGAGCATTAGCAGCTATTTAAATTGGTATCAGCAGAAACCAGG | | ISSYLNWYQQKPGKAPKLLIYAASVLQS | | | |
| | GAAAGCCCCTAAACTCCTGATCTATGCTGCATCCGTTTTGCAAAGTGGGGTCCCATCAAGG | | GVPSRFSGSGSETDFTLTISSLQPEDFA | | | |
| | TTCAGTGGCAGTGGATCTGAGACAGATTTCACTCTCACGATCAGTAGTCTGCAACCTGAAG | | TYYCQQSYSNFRTFGQGTKVEIK | | | |
| | ATTTTGCAACTTACTACTGTCAACAGAGTTACAGTAATTTTCGGACTTTTGGCCAGGGGAC | | | | | |
| | CAAGGTGGAGATCAAA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75777 | IGKV3D- | ETTLTQ | 2022 | RASQSVS | 2227 | WYQQKP | 2258 | GASSRA | 2464 | GIPARFSG | 2677 | QQYNN | 2897 | FGQGTK | 2937 |
| | 15 | SPATLS | | SNLA | | GQAPRL | | T | | SGSGTEFT | | WPRT | | VEIK | |
| | | VSPGER | | | | LIY | | | | LTISSLQS | | | | | |
| | | ATLSC | | | | | | | | EDFAVYHC | | | | | |
| ADI-75781 | IGKV1D- | DIVMTQ | 1902 | RASQSIG | 2120 | WYQQKP | 2257 | AASSLP | 2509 | GVPSRFSG | 2678 | QQSYS | 2898 | FGQGTR | 2940 |
| | 39 | SPSSLS | | SYLN | | GKAPKL | | S | | SGFGTDFT | | APPNT | | LEIK | |
| | | ASVGDR | | | | LIY | | | | LTISSLQP | | | | | |
| | | VTITC | | | | | | | | EDFATYYC | | | | | |
| ADI-75752 | IGLV3-21 | SHVLTQ | 2023 | GGTNIGG | 2228 | WYQQKP | 2283 | DDGDRP | 2510 | GIPGRISG | 2679 | QVWHS | 2899 | FGGGTK | 2939 |
| | | PPSVSA | | RSVH | | GQAPVL | | S | | SNSGNTAT | | SSDSW | | LTVL | |
| | | APGQTA | | | | VVY | | | | LTISRVVA | | V | | | |
| | | RITQ | | | | | | | | GDEADYYC | | | | | |
| ADI-75676 | IGKV4-1 | DIPMTQ | 1978 | KSSQNVL | 2229 | WYQQKP | 2290 | WASSRE | 2511 | GVPDRFSG | 2680 | QQYYS | 2832 | FGPGTK | 2942 |
| | | SPDSLA | | YSSNNKN | | GQPPKL | | S | | SGSGTDFT | | SPPT | | VEIK | |
| | | VSLGER | | YLA | | LIY | | | | LTITRLQA | | | | | |
| | | ATINC | | | | | | | | EDVAVYYC | | | | | |
| ADI-75731 | IGLV1-40 | QSVVTQ | 1972 | TGTNSNI | 2171 | WYQQLP | 2314 | ANINRP | 2512 | GVPDRFSG | 2599 | QSYDS | 2900 | FGGRTK | 2961 |
| | | PPSVSG | | GAGYDVH | | GTAPKL | | S | | SKSGTSAS | | SLTAP | | LTVL | |
| | | APGQRV | | | | LIY | | | | LAITGLQA | | L | | | |
| | | TISC | | | | | | | | EDEADYYC | | | | | |
| ADI-75837 | IGLV3-21 | SYELTQ | 2024 | GGNNIGS | 2208 | WYQQKP | 2283 | DDSDRP | 2399 | GIPERFSG | 2545 | QVWDT | 2901 | FGGGTK | 2939 |
| | | PPSVSV | | KSVH | | GQAPVL | | S | | SNSGNTAT | | SSDHQ | | LTVL | |
| | | APGQTA | | | | VVY | | | | LTISRVEA | | V | | | |
| | | MITC | | | | | | | | GDEADYYC | | | | | |
| ADI-75728 | IGKV3-20 | TMTLTQ | 2025 | RASQSVS | 2230 | WYQQKP | 2258 | GASSRA | 2513 | GIPDRFIG | 2681 | QQYGS | 2902 | FGPGTK | 2942 |
| | | SPGTLS | | SSYLA | | GQAPRL | | A | | SGSGTDFT | | SPPFT | | VEIK | |
| | | LSPGER | | | | LIY | | | | LTISRLEP | | | | | |
| | | ATLSC | | | | | | | | EDFAVYYC | | | | | |
| ADI-75601 | IGKV1D- | DIQLTQ | 2026 | RASQTVR | 2231 | WYQQKP | 2288 | AA--- | 2514 | GDPPRFSA | 2682 | QQSYS | 2820 | FGQGTK | 2937 |
| | 39 | SPSSLS | | TFLN | | GKAPKL | | PG | | TGSGTDFT | | IPYT | | VEIK | |
| | | ASVGDR | | | | LVF | | | | LTISSLQP | | | | | |
| | | VSITC | | | | | | | | EDFAIYFC | | | | | |
| ADI-75870 | IGLV1-44 | SYELTQ | 1962 | SGSSSNI | 2232 | WYQQLP | 2370 | NYNQRP | 2515 | GVPDRFSG | 2615 | AAWDD | 2813 | FGGGTK | 2939 |
| | | PPSASG | | GSNAVN | | GTAPKL | | S | | SKSGTSAS | | SLNGV | | LTVL | |
| | | TPGQRV | | | | LMY | | | | LAISGLQS | | V | | | |
| | | TISC | | | | | | | | EDEADYYC | | | | | |
| ADI-75643 | IGLV1-40 | QSVLTQ | 2027 | TGSNSNI | 2143 | WYQQLP | 2314 | GNTNRP | 2484 | GVPDRFSG | 2622 | QSYDS | 2903 | FGGGTK | 2939 |
| | | PPSVSG | | GAGYDVH | | GTAPKL | | S | | SKSDTSAS | | GLTGS | | LTVL | |
| | | APGQRV | | | | LIY | | | | LAITGLQA | | L | | | |
| | | IISC | | | | | | | | EDEADYYC | | | | | |
| ADI-75740 | IGKV2D- | DIVMTQ | 1984 | RSSQSLL | 2233 | WYLQKP | 2260 | LGSNRA | 2387 | GVPDRFSG | 2533 | MQALQ | 2904 | FGQGTK | 2937 |
| | 28 | SPLSLP | | HSNGYKY | | GQSPQL | | S | | SGSGTDFT | | APPT | | VEIK | |
| | | VTPGEP | | LD | | LIY | | | | LKISRVEA | | | | | |
| | | ASISC | | | | | | | | EDVGVYYC | | | | | |
| ADI-75720 | IGKV1D- | DITLTQ | 1900 | RASQSVS | 2234 | WYQQKP | 2371 | AASSLQ | 2391 | GVPLRFSV | 2683 | QQSYS | 2905 | FGGGTK | 2936 |
| | 39 | SPSSLS | | NYLN | | GKAPEL | | S | | SGSGTAFN | | TPLT | | VEIK | |
| | | ASVGDR | | | | LIY | | | | LTISSLQP | | | | | |
| | | VTITC | | | | | | | | GDFATYYC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75777 | GAAACGACACTCACGCAGTCTCCAGCCACCCTGTCTGTGTCTCCAGGAGAAAGAGCCACCC | 3195 | ETTLTQSPATLSVSPGERATLSCRASQS | 3477 | | |
| | TCTCCTGTAGGGCCAGTCAGAGTGTTAGCAGCAACTTAGCCTGGTACCAACAGAAACCTGG | | VSSNLAWYQQKPGQAPRLLIYGASSRAT | | | |
| | CCAGGCTCCCAGGCTCCTCATCTATGGTGCATCCAGCAGGGCCACTGGTATCCCAGCCAGG | | GIPARFSGSGSGTEFTLTISSLQSEDFA | | | |
| | TTCAGTGGCAGTGGGTCTGGGACAGAGTTCACTCTCACCATCAGCAGCCTGCAGTCTGAAG | | VYHCQQYNNWPRTFGQGTKVEIK | | | |
| | ATTTTGCAGTTTATCACTGTCAGCAGTATAATAACTGGCCTAGAACTTTTGGCCAGGGGAC | | | | | |
| | CAAGGTGGAGATCAAA | | | | | |
| ADI-75781 | GACATCGTGATGACCCAGTCTCCATCCTCCCTGTCTGCATCTGTGGGAGACAGAGTCACCA | 3196 | DIVMTQSPSSLSASVGDRVTITCRASQS | 3478 | | |
| | TCACTTGCCGGGCAAGTCAGAGCATTGGCAGCTATTTAAATTGGTATCAGCAAAAACCAGG | | IGSYLNWYQQKPGKAPKLLIYAASSLPS | | | |
| | GAAAGCCCCTAAGCTCCTGATCTATGCTGCATCCAGTTTGCCAAGTGGGGTCCCATCAAGG | | GVPSRFSGSGFGTDFTLTISSLQPEDFA | | | |
| | TTCAGTGGCAGCGGATTTGGGACAGATTTCACTCTCACCATCAGCAGTCTGCAACCTGAGG | | TYYCQQSYSAPPNTFGQGTRLEIK | | | |
| | ATTTTGCAACTTACTACTGTCAACAGAGTTACAGTGCCCCTCCGAACACCTTCGGCCAAGG | | | | | |
| | GACACGACTGGAGATTAAA | | | | | |
| ADI-75752 | TCGCATGTGCTGACTCAGCCACCCTCGGTGTCAGCGGCCCCAGGACAGACGGCCAGGATTA | 3197 | SHVLTQPPSVSAAPGQTARITCGGTNIG | 3479 | | |
| | CCTGTGGGGGAACCAATATTGGAGGTAGAAGTGTGCACTGGTACCAGCAGAAGCCAGGCCA | | GRSVHWYQQKPGQAPVLVVYDDGDRPSG | | | |
| | GGCCCCTGTGCTGGTCGTCTATGATGATGGCGACCGGCCCTCAGGGATCCCTGGGCGAATC | | IPGRISGSNSGNTATLTISRVVAGDEAD | | | |
| | TCTGGCTCCAACTCTGGGAACACGGCCACCCTGACCATCAGCAGGGTCGTAGCCGGGGATG | | YYCQVWHSSSDSWVFGGGTKLTVL | | | |
| | AGGCCGACTATTACTGTCAGGTGTGGCATAGTAGTAGTGATTCCTGGGTGTTCGGCGGAGG | | | | | |
| | GACCAAGCTCACCGTCCTA | | | | | |
| ADI-75676 | GACATCCCGATGACCCAGTCTCCAGACTCCCTGGCTGTGTCTCTGGGCGAGAGGGCCACCA | 3198 | DIPMTQSPDSLAVSLGERATINCKSSQN | 3480 | | |
| | TCAACTGCAAGTCCAGCCAGAATGTTTTATACAGCTCCAACAATAAGAACTACTTAGCTTG | | VLYSSNNKNYLAWYQQKPGQPPKLLIYW | | | |
| | GTACCAGCAGAAACCAGGACAGCCTCCTAAGCTGCTCATTTACTGGGCATCTTCCCGGGAA | | ASSRESGVPDRFSGSGSGTDFTLTITRL | | | |
| | TCCGGGGTCCCTGACCGATTCAGTGGCAGCGGGTCTGGGACAGATTTCACTCTCACCATCA | | QAEDVAVYYCQQYYSSPPTFGPGTKVEI | | | |
| | CCCGCCTGCAGGCTGAAGATGTGGCAGTGTATTACTGTCAGCAATATTATAGTAGTCCTCC | | K | | | |
| | CACTTTCGGCCCTGGGACCAAGGTGGAGATCAAA | | | | | |
| ADI-75731 | CAGTCTGTGGTGACGCAGCCGCCCTCAGTGTCTGGGGCCCCAGGGCAGAGGGTCACCATCT | 3199 | QSVVTQPPSVSGAPGQRVTISCTGTNSN | 3481 | | |
| | CCTGCACTGGGACCAACTCCAACATCGGGGCCGGTTATGATGTACACTGGTACCAGCAGCT | | IGAGYDVHWYQQLPGTAPKLLIYANINR | | | |
| | TCCAGGAACAGCCCCCAAACTCCTCATCTATGCTAACATCAATCGGCCCTCGGGGGTCCCT | | PSGVPDRFSGSKSGTSASLAITGLQAED | | | |
| | GACCGATTCTCTGGCTCCAAGTCTGGCACCTCAGCCTCCCTGGCCATCACTGGGCTCCAGG | | EADYYCQSYDSSLTAPLFGGRTKLTVL | | | |
| | CTGAGGATGAGGCTGATTATTACTGCCAGTCCTATGACAGCAGCCTGACTGCTCCGTTATT | | | | | |
| | CGGCGGACGGACCAAGCTCACCGTCCTC | | | | | |
| ADI-75837 | TCGCATGTGCTGACTCAGCCACCCTCGGTGTCAGCGGCCCCAGGACAGACGGCCAGGATTA | 3200 | SYELTQPPSVSVAPGQTAMITCGGNNIG | 3482 | | |
| | CCTGTGGGGGAACCAATATTGGAGGTAGAAGTGTGCACTGGTACCAGCAGAAGCCAGGCCA | | SKSVHWYQQKPGQAPVLVVYDDSDRPSG | | | |
| | GGCCCCTGTGCTGGTCGTCTATGATGATGGCGACCGGCCCTCAGGGATCCCTGGGCGAATC | | IPERFSGSNSGNTATLTISRVEAGDEAD | | | |
| | TCTGGCTCCAACTCTGGGAACACGGCCACCCTGACCATCAGCAGGGTCGTAGCCGGGGATG | | YYCQVWDTSSDHQVFGGGTKLTVL | | | |
| | AGGCCGACTATTACTGTCAGGTGTGGCATAGTAGTAGTGATTCCTGGGTGTTCGGCGGAGG | | | | | |
| | GACCAAGCTCACCGTCCTA | | | | | |
| ADI-75728 | ACAATGACACTGACGCAGTCTCCAGGCACCCTGTCTTTGTCTCCAGGGGAAAGAGCCACCC | 3201 | TMTLTQSPGTLSLSPGERATLSCRASQS | 3483 | | |
| | TCTCCTGCAGGGCCAGTCAGAGTGTTAGCAGCAGCTACTTAGCCTGGTATCAGCAGAAACC | | VSSSYLAWYQQKPGQAPRLLIYGASSRA | | | |
| | TGGCCAGGCTCCCAGGCTCCTCATCTATGGTGCATCCAGCAGGGCCGCTGGCATCCCAGAC | | AGIPDRFIGSGSGTDFTLTISRLEPEDF | | | |
| | AGATTCATTGGCAGTGGGTCTGGGACAGACTTCACTCTCACCATCAGCAGGCTGGAGCCTG | | AVYYCQQYGSSPPFTFGPGTKVEIK | | | |
| | AGGATTTTGCAGTGTATTACTGTCAGCAGTATGGTAGCTCACCTCCATTCACTTTCGGCCC | | | | | |
| | TGGGACCAAGGTGGAAATCAAA | | | | | |
| ADI-75601 | GAAACGACACTCACGCAGTCTCCAGCCACCCTGTCTGTGTCTCCAGGAGAAAGAGCCACCC | 3202 | DIQLTQSPSSLSASVGDRVSITCRASQT | 3484 | | |
| | TCTCCTGTAGGGCCAGTCAGAGTGTTAGCAGCAACTTAGCCTGGTACCAACAGAAACCTGG | | VRTFLNWYQQKPGKAPKLLVFAAPGGDP | | | |
| | CCAGGCTCCCAGGCTCCTCATCTATGGTGCATCCAGCAGGGCCACTGGTATCCCAGCCAGG | | PRFSATGSGTDFTLTISSLQPEDFAIYF | | | |
| | TTCAGTGGCAGTGGGTCTGGGACAGAGTTCACTCTCACCATCAGCAGCCTGCAGTCTGAAG | | CQQSYSIPYTFGQGTKVEIK | | | |
| | ATTTTGCAGTTTATCACTGTCAGCAGTATAATAACTGGCCTAGAACTTTTGGCCAGGGGAC | | | | | |
| | CAAGGTGGAGATCAAA | | | | | |
| ADI-75870 | TCGTATGAGCTGACTCAGCCACCCTCAGCGTCTGGGACCCCCGGGCAGAGGGTCACCATCT | 3203 | SYELTQPPSASGTPGQRVTISCSGSSSN | 3485 | | |
| | CTTGTTCTGGGAGCAGCTCCAACATCGGAAGTAATGCTGTAAACTGGTACCAGCAGCTCCC | | IGSNAVNWYQQLPGTAPKLLMYNYNQRP | | | |
| | AGGAACGGCCCCCAAACTCCTCATGTATAATTATAATCAGCGGCCCTCAGGGGTCCCTGAC | | SGVPDRFSGSKSGTSASLAISGLQSEDE | | | |
| | CGATTCTCTGGCTCCAAGTCTGGCACCTCAGCCTCCCTGGCCATCAGTGGGCTCCAGTCTG | | ADYYCAAWDDSLNGVVFGGGTKLTVL | | | |
| | AGGATGAGGCTGATTATTACTGTGCAGCATGGGATGACAGCCTGAATGGTGTGGTTTTCGG | | | | | |
| | CGGAGGGACCAAGCTCACCGTCCTA | | | | | |
| ADI-75643 | CAGTCTGTGTTGACGCAGCCGCCCTCAGTGTCTGGGGCCCCAGGGCAGAGGGTCATCATCT | 3204 | QSVLTQPPSVSGAPGQRVIISCTGSNSN | 3486 | | |
| | CCTGCACTGGGAGCAACTCCAACATCGGGGCAGGTTATGATGTACACTGGTACCAGCAGCT | | IGAGYDVHWYQQLPGTAPKLLIYGNTNR | | | |
| | TCCAGGAACAGCCCCCAAACTCCTCATCTATGGTAACACCAATCGGCCCTCAGGGGTCCCT | | PSGVPDRFSGSKSDTSASLAITGLQAED | | | |
| | GACCGGTTCTCTGGCTCCAAGTCTGACACCTCAGCCTCCCTGGCCATCACTGGACTCCAGG | | EADYYCQSYDSGLTGSLFGGGTKLTVL | | | |
| | CTGAGGATGAGGCTGATTATTACTGCCAGTCCTATGACAGCGGCCTGACTGGTTCGCTTTT | | | | | |
| | CGGCGGAGGGACCAAGCTGACCGTCCTA | | | | | |
| ADI-75740 | GACATTGTGATGACTCAGTCTCCACTCTCCCTGCCCGTCACCCCTGGAGAGCCGGCCTCCA | 3205 | DIVMTQSPLSLPVTPGEPASISCRSSQS | 3487 | | |
| | TCTCCTGCAGGTCTAGTCAGAGCCTCCTGCATAGTAATGGATACAAGTATTTGGATTGGTA | | LLHSNGYKYLDWYLQKPGQSPQLLIYLG | | | |
| | CCTGCAGAAGCCAGGGCAGTCTCCACAGCTCCTGATCTATTTGGGTTCTAATCGGGCCTCC | | SNRASGVPDRFSGSGSGTDFTLKISRVE | | | |
| | GGGGTCCCTGACAGGTTCAGTGGCAGTGGATCAGGCACAGATTTTACACTGAAAATCAGCA | | AEDVGVYYCMQALQAPPTFGQGTKVEIK | | | |
| | GAGTGGAGGCTGAGGATGTTGGGGTTTATTACTGCATGCAAGCTCTACAAGCTCCTCCGAC | | | | | |
| | GTTCGGCCAAGGGACCAAGGTGGAGATCAAA | | | | | |
| ADI-75720 | GACATCACACTGACGCAGTCTCCATCCTCCCTGTCTGCATCTGTAGGGGACAGAGTCACCA | 3206 | DITLTQSPSSLSASVGDRVTITCRASQS | 3488 | | |
| | TCACTTGCCGGGCAAGTCAGAGCGTTAGCAATTATTTAAATTGGTATCAGCAAAAACCAGG | | VSNYLNWYQQKPGKAPELLIYAASSLQS | | | |
| | GAAAGCCCCTGAGCTCCTGATCTATGCTGCATCCAGTTTGCAAAGTGGCGTCCCATTAAGG | | GVPLRFSVSGSGTAFNLTISSLQPGDFA | | | |
| | TTCAGTGTCAGTGGATCTGGGACAGCTTTCAATCTCACAATCAGTAGTCTGCAGCCTGGAG | | TYYCQQSYSTPLTFGGGTKVEIK | | | |
| | ATTTCGCAACTTACTACTGTCAACAGAGTTACAGTACCCCGCTCACTTTCGGCGGAGGGAC | | | | | |
| | CAAGGTGGAAATCAAA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75794 | IGLV7-43 | QTVVTQ | 2028 | TNGYYPT | 2235 | WFQQKP | 2372 | STNNKH | 2516 | WTPARFSG | 2684 | LLYYG | 2906 | FGGGTK | 2939 |
| | | EPSLTV | | ASSTGTV | | GQAPRA | | S | | SLLGGKAA | | GVRV | | LTVL | |
| | | SPGGTV | | | | LIY | | | | LTLSGVQP | | | | | |
| | | TLTC | | | | | | | | EDEADYYC | | | | | |
| ADI-75577 | IGKV1D- | EIPMTQ | 1912 | RASQGIS | 2236 | WYQQRP | 2309 | AASSLQ | 2391 | GVPSRFSG | 2685 | QQSYT | 2885 | FGQGTK | 2937 |
| | 39 | SPSSLS | | NYLN | | GKAPKL | | S | | SGFGTDFT | | TRWT | | VEIK | |
| | | ASVGDR | | | | LIY | | | | LTISSLQP | | | | | |
| | | VTITC | | | | | | | | DDFATYYC | | | | | |
| ADI-75850 | IGLV3-21 | QPELTQ | 2029 | GGDNIGT | 2237 | WYQQRP | 2373 | DNSDRP | 2494 | GIPERFSG | 2584 | QVWDS | 2907 | FGGGTK | 2947 |
| | | PPSVSV | | KSVH | | GQAPVL | | S | | SNSGNTAT | | VTDHV | | VTVL | |
| | | APGQTA | | | | VVF | | | | LTISRVEA | | L | | | |
| | | RITC | | | | | | | | GDEADFYC | | | | | |
| ADI-75770 | IGLV1-40 | QSVLTQ | 1948 | TGSNSNI | 2143 | WYQQLP | 2314 | ANANRP | 2517 | GVPDRFSG | 2666 | QSYDS | 2908 | FGGGTQ | 2948 |
| | | PPSVSG | | GAGYDVH | | GTAPKL | | S | | SRSGTSAS | | SLSGS | | LTVL | |
| | | APGQRV | | | | LIY | | | | LAITGLQA | | V | | | |
| | | TISC | | | | | | | | EDEADYYC | | | | | |
| ADI-75799 | IGKV1-33 | DILMTQ | 1975 | QASQDIN | 2148 | WYQQKP | 2257 | DASNLE | 2416 | GVPSRFSG | 2537 | QQYDN | 2909 | FGQGTK | 2937 |
| | | SPSSLS | | NYLN | | GKAPKL | | T | | SGSGTDFT | | LPRT | | VEIK | |
| | | ASVGDR | | | | LIY | | | | FTISSLQP | | | | | |
| | | VTITC | | | | | | | | EDIATYYC | | | | | |
| ADI-75759 | IGKV1-5 | EITLTQ | 2030 | RASQSIR | 2238 | WYQQKP | 2257 | KASTLE | 2518 | GVPSRFSG | 2686 | QQYNT | 2910 | FGQGTK | 2937 |
| | | SPSTLS | | SWLA | | GKAPKL | | S | | SGSETEFT | | DRYT | | VEIK | |
| | | ASVGDS | | | | LIY | | | | LTIRSLQP | | | | | |
| | | VTITC | | | | | | | | EDFATYYC | | | | | |
| ADI-75803 | IGLV3-21 | TYVLTQ | 2031 | GGHNIGG | 2239 | WYQQKP | 2283 | DDGDRP | 2510 | GIPERISG | 2687 | QVWHS | 2899 | FGGGTK | 2939 |
| | | PPSVSV | | KSVH | | GQAPVL | | S | | SNSGNTAT | | SSDSW | | LTVL | |
| | | APGQPA | | | | VVY | | | | LTISRVEA | | V | | | |
| | | RITC | | | | | | | | GDEADYYC | | | | | |
| ADI-75710 | IGLV1-40 | QSVLTQ | 1948 | TGTNSNI | 2171 | WYQQLP | 2314 | GNINRP | 2519 | GVPDRFSG | 2688 | QSYDS | 2911 | FGGRTK | 2961 |
| | | PPSVSG | | GAGYDVH | | GTAPKL | | S | | SKSGSSAS | | SLSGS | | LTVL | |
| | | APGQRV | | | | LIY | | | | LAITGLQA | | L | | | |
| | | TISC | | | | | | | | EDEADYYC | | | | | |
| ADI-75605 | IGLV3-21 | QHVLTQ | 2032 | GGNNIGS | 2208 | WYQQKP | 2283 | DDSDRP | 2399 | GIPERFSG | 2689 | QVWDS | 2912 | FGGGTK | 2939 |
| | | PPSVSV | | KSVH | | GQAPVL | | S | | SNSGTTAT | | NNDHV | | LTVL | |
| | | APGQTA | | | | VVY | | | | LTISRVEA | | V | | | |
| | | RITC | | | | | | | | GDEADYFC | | | | | |
| ADI-75680 | IGKV3D- | EIVLTQ | 2033 | RASQSVS | 2240 | WYQQQP | 2374 | DASTRA | 2424 | GIPARFSG | 2690 | QQYND | 2913 | FGGGTK | 2936 |
| | 15 | SPATLS | | SDLA | | GQAPRL | | T | | SGSGTEFT | | WLT | | VEIK | |
| | | VSPGER | | | | LIY | | | | LTISSLQS | | | | | |
| | | ATLSC | | | | | | | | EDFVVYYC | | | | | |
| ADI-75691 | IGKV3D- | EIVMTQ | 2034 | RASQSVS | 2240 | WYQQQP | 2374 | DASTRA | 2424 | GIPARFSG | 2690 | QQYND | 2913 | FGGGTK | 2936 |
| | 15 | SPATLS | | SDLA | | GQAPRL | | T | | SGSGTEFT | | WLT | | VEIK | |
| | | VSPGER | | | | LIY | | | | LTISSLQS | | | | | |
| | | ATLSC | | | | | | | | EDFVVYYC | | | | | |
| ADI-75573 | IGKV1D- | DIQLTQ | 1876 | RASQSIS | 2077 | WYQQKP | 2375 | AASSLQ | 2391 | GVPSRFSG | 2691 | QQSYS | 2914 | FGQGTK | 2937 |
| | 39 | SPSSLS | | NYLN | | GKAPKL | | S | | SGSGTDFT | | TPVT | | VEIK | |
| | | ASVGDR | | | | QIY | | | | LTISSLQV | | | | | |
| | | VTITC | | | | | | | | EDFATYYC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75794 | CAGACTGTGGTGACCCAGGAGCCCTCACTGACTGTGTCCCCAGGAGGGACAGTCACTCTCA | 3207 | QTVVTQEPSLTVSPGGTVTLTCASSTGT | 3489 | | |
| | CCTGTGCTTCCAGCACTGGAACAGTCACCAATGGTTACTATCCAACCTGGTTCCAGCAGAA | | VTNGYYPTWFQQKPGQAPRALIYSTNNK | | | |
| | ACCTGGACAAGCACCCAGGGCATTGATTTATAGTACAAACAACAAACACTCCTGGACCCCT | | HSWTPARFSGSLLGGKAALTLSGVQPED | | | |
| | GCCCGGTTCTCAGGCTCCCTCCTTGGGGGCAAAGCTGCCCTGACACTGTCAGGTGTGCAGC | | EADYYCLLYYGGVRVFGGGTKLTVL | | | |
| | CTGAGGACGAGGCTGACTATTACTGCCTGCTCTACTACGGTGGTGTTCGAGTCTTCGGCGG | | | | | |
| | AGGGACCAAGCTGACCGTCCTA | | | | | |
| ADI-75577 | GAAATCCCAATGACACAGTCTCCATCCTCCCTGTCTGCATCTGTAGGAGACAGAGTCACCA | 3208 | EIPMTQSPSSLSASVGDRVTITCRASQG | 3490 | | |
| | TCACTTGCCGGGCAAGTCAGGGCATTAGCAACTATTTAAATTGGTATCAACAGAGACCAGG | | ISNYLNWYQQRPGKAPKLLIYAASSLQS | | | |
| | GAAAGCCCCTAAACTCCTTATCTATGCTGCATCCAGTTTGCAAAGTGGGGTCCCATCAAGG | | GVPSRFSGSGFGTDFTLTISSLQPDDFA | | | |
| | TTCAGTGGCAGTGGATTTGGGACAGATTTCACTCTCACCATCAGCAGTCTGCAACCTGACG | | TYYCQQSYTTRWTFGQGTKVEIK | | | |
| | ACTTTGCAACTTACTACTGTCAACAGAGTTACACTACCCGGTGGACGTTCGGCCAAGGGAC | | | | | |
| | CAAGGTGGAGATCAAA | | | | | |
| ADI-75850 | CAGCCTGAGCTGACTCAGCCACCCTCGGTGTCAGTGGCCCCAGGACAGACGGCCAGGATTA | 3209 | QPELTQPPSVSVAPGQTARITCGGDNIG | 3491 | | |
| | CCTGTGGGGGTGACAACATTGGAACTAAAAGTGTGCACTGGTACCAGCAGAGGCCAGGCCA | | TKSVHWYQQRPGQAPVLVVFDNSDRPSG | | | |
| | GGCCCCTGTGCTGGTCGTCTTTGATAATTCCGACCGGCCCTCAGGGATCCCTGAGCGATTC | | IPERFSGSNSGNTATLTISRVEAGDEAD | | | |
| | TCTGGCTCCAATTCTGGGAACACGGCCACCCTGACCATCAGCAGGGTCGAAGCCGGGGATG | | FYCQVWDSVTDHVLFGGGTKVTVL | | | |
| | AGGCCGACTTTTACTGTCAGGTGTGGGATAGTGTTACTGATCATGTGCTTTTCGGCGGAGG | | | | | |
| | GACCAAGGTCACCGTCCTA | | | | | |
| ADI-75770 | CAGTCTGTGTTGACGCAGCCGCCCTCAGTGTCTGGGGCCCCAGGGCAGAGGGTCACCATCT | 3210 | QSVLTQPPSVSGAPGQRVTISCTGSNSN | 3492 | | |
| | CCTGCACTGGGAGCAACTCCAACATCGGGGCAGGTTATGATGTACACTGGTACCAGCAGCT | | IGAGYDVHWYQQLPGTAPKLLIYANANR | | | |
| | TCCAGGAACAGCCCCCAAACTCCTCATCTATGCTAACGCCAATCGGCCCTCAGGGGTCCCT | | PSGVPDRFSGSRSGTSASLAITGLQAED | | | |
| | GACCGATTCTCTGGCTCCAGGTCTGGCACCTCAGCCTCCCTGGCCATCACTGGGCTCCAGG | | EADYYCQSYDSSLSGSVFGGGTQLTVL | | | |
| | CTGAGGATGAGGCTGATTATTACTGTCAGTCCTATGACAGCAGCCTGAGTGGTTCGGTGTT | | | | | |
| | CGGCGGAGGCACCCAGCTGACCGTCCTC | | | | | |
| ADI-75799 | GACATCCTGATGACCCAGTCTCCATCCTCCCTGTCTGCATCTGTAGGAGACAGAGTCACCA | 3211 | DILMTQSPSSLSASVGDRVTITCQASQD | 3493 | | |
| | TCACTTGCCAGGCGAGTCAGGACATTAACAACTATTTAAATTGGTATCAGCAGAAACCAGG | | INNYLNWYQQKPGKAPKLLIYDASNLET | | | |
| | GAAAGCCCCTAAGCTCCTGATCTACGATGCATCCAATTTGGAAACAGGGGTCCCATCAAGG | | GVPSRFSGSGSGTDFTFTISSLQPEDIA | | | |
| | TTCAGTGGAAGTGGATCTGGGACAGATTTTACTTTCACCATCAGCAGCCTGCAGCCTGAAG | | TYYCQQYDNLPRTFGQGTKVEIK | | | |
| | ATATTGCAACATATTACTGTCAACAGTATGATAATCTCCCTCGGACGTTCGGCCAAGGGAC | | | | | |
| | CAAGGTGGAAATCAAA | | | | | |
| ADI-75759 | GAAATCACACTGACCCAGTCTCCTTCCACCCTGTCTGCATCTGTGGGAGACAGCGTCACTA | 3212 | EITLTQSPSTLSASVGDSVTITCRASQS | 3494 | | |
| | TCACTTGTCGGGCCAGTCAGAGTATTAGAAGCTGGTTGGCCTGGTATCAGCAGAAACCAGG | | IRSWLAWYQQKPGKAPKLLIYKASTLES | | | |
| | GAAAGCCCCTAAGCTCCTGATCTATAAGGCGTCTACTTTAGAAAGTGGGGTCCCATCAAGG | | GVPSRFSGSGSETEFTLTIRSLQPEDFA | | | |
| | TTCAGCGGCAGTGGATCTGAGACAGAATTCACACTCACCATCAGAAGCCTGCAGCCTGAAG | | TYYCQQYNTDRYTFGQGTKVEIK | | | |
| | ATTTTGCAACTTATTACTGCCAACAGTATAATACTGATCGGTACACTTTTGGCCAGGGGAC | | | | | |
| | CAAGGTGGAAATCAAA | | | | | |
| ADI-75803 | ACGTATGTGCTGACTCAGCCACCCTCGGTGTCAGTGGCCCCAGGACAGCCGGCCAGGATTA | 3213 | TYVLTQPPSVSVAPGQPARITCGGHNIG | 3495 | | |
| | CCTGTGGGGGACACAATATTGGAGGTAAAAGTGTGCACTGGTACCAGCAGAAGCCAGGCCA | | GKSVHWYQQKPGQAPVLVVYDDGDRPSG | | | |
| | GGCCCCTGTGCTGGTCGTCTATGATGATGGCGACCGGCCCTCAGGGATCCCTGAGCGAATC | | IPERISGSNSGNTATLTISRVEAGDEAD | | | |
| | TCTGGCTCCAACTCTGGGAACACGGCCACCCTGACCATCAGCAGGGTCGAAGCCGGGGATG | | YYCQVWHSSSDSWVFGGGTKLTVL | | | |
| | AGGCCGACTATTACTGTCAGGTGTGGCATAGTAGTAGTGATTCCTGGGTGTTCGGCGGAGG | | | | | |
| | GACCAAGCTCACCGTCCTA | | | | | |
| ADI-75710 | CAGTCTGTCTTGACGCAGCCGCCCTCAGTGTCTGGGGCCCCAGGGCAGAGGGTCACCATCT | 3214 | QSVLTQPPSVSGAPGQRVTISCTGTNSN | 3496 | | |
| | CCTGCACTGGGACCAACTCCAACATCGGGGCAGGTTATGATGTACACTGGTACCAGCAGCT | | IGAGYDVHWYQQLPGTAPKLLIYGNINR | | | |
| | TCCAGGAACAGCCCCCAAACTCCTCATCTATGGTAACATCAATCGGCCCTCAGGGGTCCCT | | PSGVPDRFSGSKSGSSASLAITGLQAED | | | |
| | GACCGATTCTCTGGCTCCAAGTCTGGCTCCTCTGCCTCCCTGGCCATCACTGGGCTCCAGG | | EADYYCQSYDSSLSGSLFGGRTKLTVL | | | |
| | CTGAGGATGAGGCTGATTATTACTGCCAGTCCTATGACAGCAGTCTGAGTGGTTCGTTATT | | | | | |
| | CGGCGGACGCACCAAGCTGACCGTCCTA | | | | | |
| ADI-75605 | CAGCATGTGCTGACTCAGCCGCCCTCGGTGTCAGTGGCCCCAGGACAGACGGCCAGGATTA | 3215 | QHVLTQPPSVSVAPGQTARITCGGNNIG | 3497 | | |
| | CCTGTGGGGGCAACAACATTGGAAGTAAAAGTGTGCACTGGTACCAGCAGAAGCCAGGCCA | | SKSVHWYQQKPGQAPVLVVYDDSDRPSG | | | |
| | GGCCCCTGTGCTGGTCGTCTATGATGATAGCGACCGGCCCTCAGGGATCCCTGAGCGATTT | | IPERFSGSNSGTTATLTISRVEAGDEAD | | | |
| | TCTGGCTCCAACTCTGGGACCACGGCCACCCTGACCATCAGCAGGGTCGAGGCCGGGGATG | | YFCQVWDSNNDHVVFGGGTKLTVL | | | |
| | AGGCCGACTATTTTTGTCAGGTGTGGGATAGTAATAATGATCATGTGGTATTCGGCGGAGG | | | | | |
| | GACCAAGCTCACCGTCCTA | | | | | |
| ADI-75680 | GAAATTGTGCTGACTCAGTCTCCAGCCACCCTGTCTGTGTCTCCAGGGGAAAGAGCCACCC | 3216 | EIVLTQSPATLSVSPGERATLSCRASQS | 3498 | | |
| | TCTCCTGCAGGGCCAGTCAGAGTGTTAGCAGCGACTTAGCCTGGTACCAGCAGCAACCTGG | | VSSDLAWYQQQPGQAPRLLIYDASTRAT | | | |
| | CCAGGCTCCCAGACTCCTCATCTATGATGCATCCACCAGGGCCACTGGTATCCCAGCCAGG | | GIPARFSGSGSGTEFTLTISSLQSEDFV | | | |
| | TTCAGTGGCAGTGGGTCTGGGACAGAGTTCACTCTCACCATCAGCAGCCTGCAGTCTGAAG | | VYYCQQYNDWLTFGGGTKVEIK | | | |
| | ATTTTGTAGTTTATTACTGTCAGCAGTACAATGACTGGCTCACTTTCGGGGAGGGACCAA | | | | | |
| | GGTGGAAATCAAA | | | | | |
| ADI-75691 | GAAATTGTGATGACGCAGTCTCCAGCCACCCTGTCTGTGTCTCCAGGGGAAAGAGCCACCC | 3217 | EIVMTQSPATLSVSPGERATLSCRASQS | 3499 | | |
| | TCTCCTGCAGGGCCAGTCAGAGTGTTAGCAGCGACTTAGCCTGGTACCAGCAGCAACCTGG | | VSSDLAWYQQQPGQAPRLLIYDASTRAT | | | |
| | CCAGGCTCCCAGACTCCTCATCTATGATGCATCCACCAGGGCCACTGGTATCCCAGCCAGG | | GIPARFSGSGSGTEFTLTISSLQSEDFV | | | |
| | TTCAGTGGCAGTGGGTCTGGGACAGAGTTCACTCTCACCATCAGCAGCCTGCAGTCTGAAG | | VYYCQQYNDWLTFGGGTKVEIK | | | |
| | ATTTTGTAGTTTATTACTGTCAGCAGTACAATGACTGGCTCACTTTCGGCGGAGGGACCAA | | | | | |
| | GGTGGAAATCAAA | | | | | |
| ADI-75573 | GACATCCAGTTGACCCAGTCTCCATCCTCCCTGTCTGCATCTGTAGGAGACAGAGTCACCA | 3218 | DIQLTQSPSSLSASVGDRVTITCRASQS | 3500 | | |
| | TCACTTGCCGGGCAAGTCAGAGCATTAGCAATTATTTAAATTGGTATCAGCAGAAACCAGG | | ISNYLNWYQQKPGKAPKLQIYAASSLQS | | | |
| | GAAAGCCCCTAAGCTCCAGATCTATGCTGCATCCAGTTTGCAAAGTGGGGTCCCATCAAGG | | GVPSRFSGSGSGTDFTLTISSLQVEDFA | | | |
| | TTCAGTGGCAGTGGATCTGGGACAGATTTCACTCTCACCATCAGCAGTCTGCAAGTTGAAG | | TYYCQQSYSTPVTFGQGTKVEIK | | | |
| | ATTTTGCAACTTATTACTGTCAACAGAGTTACAGTACCCCTGTCACTTTTGGCCAGGGGAC | | | | | |
| | CAAGGTGGAGATCAAA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75580 | IGKV2D- | VTPGEP | 1984 | RSSQSLL | 2241 | WYLQKP | 2260 | LGTNRA | 2520 | GVPDRFSG | 2575 | MQALQ | 2915 | FGGGTK | 2936 |
| | 28 | DIVMTQ | | HSNGYTY | | GQSPQL | | S | | SGSGTDFT | | TPLT | | VEIK | |
| | | SPLSLP | | LD | | LIY | | | | LKISRVEA | | | | | |
| | | ASISC | | | | | | | | EDVGIYYC | | | | | |
| ADI-75576 | IGLV3-21 | YYMLTQ | 2035 | GGNDIGG | 2242 | WYQQRP | 2277 | DDSDRP | 2399 | GIPERFSG | 2545 | QMWDS | 2916 | FGGGTK | 2947 |
| | | PPSVSV | | KAVH | | GQAPVL | | S | | SNSGNTAT | | RSDHP | | VTVL | |
| | | APGQTA | | | | VVY | | | | LTISRVEA | | V | | | |
| | | RLTC | | | | | | | | GDEADYYC | | | | | |
| ADI-75592 | IGKV1D- | DIRMTQ | 2036 | RASQSIS | 2243 | WYQQKP | 2275 | AASSLQ | 2391 | GVPSRFSG | 2692 | QQGST | 2917 | FGRGTK | 2941 |
| | 39 | SPSSLS | | TILN | | GRAPKL | | S | | GGSGTDFT | | TPQT | | VEIK | |
| | | ASIGDR | | | | LIY | | | | LTISSLQP | | | | | |
| | | VTITC | | | | | | | | EDFATYYC | | | | | |
| ADI-75597 | IGKV1-12 | DIQMTQ | 1885 | RASQGIS | 2244 | WYQQKP | 2257 | AASSLQ | 2391 | GVPSRFSG | 2534 | QQANS | 2918 | FGGGTK | 2936 |
| | | SPSSVS | | SWLA | | GKAPKL | | S | | SGSGTDFT | | FPIT | | VEIK | |
| | | ASVGDR | | | | LIY | | | | LTISSLQP | | | | | |
| | | VTITC | | | | | | | | EDFATYYC | | | | | |
| ADI-75636 | IGKV1D- | EIVMTQ | 1891 | RASHRID | 2098 | WYQQKP | 2291 | VASTLQ | 2423 | GVPSRFTG | 2551 | QQSYT | 2752 | FGPGTK | 2942 |
| | 39 | SPSSLS | | RYLN | | GQAPKL | | S | | SGSGTDFT | | TPRT | | VEIK | |
| | | ASVGDR | | | | LIY | | | | LTISSLQP | | | | | |
| | | VTITC | | | | | | | | EDFATYYC | | | | | |
| ADI-75645 | IGKV2-40 | EIVMTQ | 1923 | RSSQSLL | 2245 | WYLQKP | 2376 | TLSYRA | 2521 | GVPDRFSG | 2693 | MQRIE | 2919 | FGQGTK | 2937 |
| | | SPLSLP | | DSDDVYT | | GQSPQL | | S | | SGSGTNFT | | FPYT | | VEIK | |
| | | VTPGEP | | YLD | | LMY | | | | LKINRVKT | | | | | |
| | | ASISC | | | | | | | | EDVGVYYC | | | | | |
| ADI-75615 | IGLV3-1 | PSELTQ | 2037 | SGENLGR | 2246 | WYQQRS | 2377 | RDDKRP | 2522 | GIPERFSG | 2671 | QAWDS | 2920 | FGTGTK | 2945 |
| | | PPSVSV | | KYVC | | GQSPVL | | S | | SNSGNTAT | | RTAQV | | VTVL | |
| | | SPGQTA | | | | VIY | | | | LTISGTQA | | | | | |
| | | SITC | | | | | | | | MDEADYYC | | | | | |
| ADI-75844 | IGKV1D- | DIQMTQ | 1878 | RASQTIN | 2247 | WYQQKP | 2263 | AASSLQ | 2391 | GVPSRFSG | 2534 | QQSYS | 2921 | FGQGTR | 2940 |
| | 39 | SPSSLS | | NYLT | | GKAPNL | | S | | SGSGTDFT | | SPIT | | LEIK | |
| | | ASVGDR | | | | LIY | | | | LTISSLQP | | | | | |
| | | VTITC | | | | | | | | EDFATYYC | | | | | |
| ADI-75875 | IGLV6-57 | KPVLTQ | 2038 | TRSSGSI | 2109 | WYQQRP | 2378 | EDNQRP | 2431 | GVPDRFSG | 2694 | QSYDS | 2922 | FGGGTK | 2939 |
| | | PHSVSE | | ASNYVQ | | GSSPST | | S | | SIDSSSNS | | SLWV | | LTVL | |
| | | SPGKTV | | | | VIY | | | | ASLTISGL | | | | | |
| | | TISC | | | | | | | | QTDDEADY | | | | | |
| | | | | | | | | | | YC | | | | | |
| ADI-75714 | IGLV1-40 | QSVLTQ | 1948 | TGSNSNI | 2143 | WYQQLP | 2314 | GNNNRP | 2492 | GVPDRFSG | 2695 | QSYDS | 2840 | FGGGTK | 2947 |
| | | PPSVSG | | GAGYDVH | | GTAPKL | | S | | SKSVTSAS | | SLSDS | | VTVL | |
| | | APGQRV | | | | LIY | | | | LAITGLQA | | V | | | |
| | | TISC | | | | | | | | EDEADYYC | | | | | |
| ADI-75688 | IGKV3-20 | EIVMTQ | 1935 | RASQSVS | 2230 | WYQQKP | 2258 | GASSRA | 2464 | GIPDRFSG | 2616 | QQYGS | 2923 | FGQGTK | 2937 |
| | | SPGTLS | | SSYLA | | GQAPRL | | T | | SGSGTDFT | | SPGT | | VEIK | |
| | | LSPGER | | | | LIY | | | | LTISRLEP | | | | | |
| | | ATLSC | | | | | | | | EDFAVYYC | | | | | |
| ADI-75637 | IGKV1D- | DITMTQ | 2039 | RASQGIS | 2248 | WCQQKP | 2379 | AASSLQ | 2391 | GVPSRFSG | 2614 | QQSYS | 2751 | FGQGTK | 2937 |
| | 39 | SPSSLS | | TSLN | | GKAPKL | | S | | SGSGTDFT | | TPWT | | VEIK | |
| | | ASVGDR | | | | LIS | | | | LTISNLQP | | | | | |
| | | VTIAC | | | | | | | | EDFATYYC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75580 | GACATTGTGATGACCCAGTCTCCACTCTCCCTGCCCGTCACCCCTGGAGAGCCGGCCTCCA | 3219 | DIVMTQSPLSLPVTPGEPASISCRSSQS | 3501 | | |
| | TCTCCTGCAGGTCTAGTCAGAGCCTCCTGCATAGTAATGGATACACCTATTTGGATTGGTA | | LLHSNGYTYLDWYLQKPGQSPQLLIYLG | | | |
| | CCTGCAGAAGCCAGGGCAGTCTCCACAGCTCCTGATCTATTTGGGTACTAATCGGGCCTCC | | TNRASGVPDRFSGSGSGTDFTLKISRVE | | | |
| | GGGGTCCCTGACAGGTTCAGTGGCAGTGGATCAGGCACAGATTTTACACTGAAAATCAGCA | | AEDVGIYYCMQALQTPLTFGGGTKVEIK | | | |
| | GAGTGGAGGCTGAAGATGTTGGGATTTATTACTGCATGCAAGCTCTACAAACTCCGCTCAC | | | | | |
| | TTTCGGCGGAGGGACCAAGGTGGAGATCAAA | | | | | |
| ADI-75576 | TATTATATGCTGACTCAGCCCCCCTCGGTGTCAGTGGCCCCAGGACAGACGGCCAGGCTTA | 3220 | YYMLTQPPSVSVAPGQTARLTCGGNDIG | 3502 | | |
| | CCTGTGGGGGAAACGACATTGGAGGTAAAGCTGTGCACTGGTACCAGCAGAGGCCAGGCCA | | GKAVHWYQQRPGQAPVLVVYDDSDRPSG | | | |
| | GGCCCCTGTGCTGGTCGTCTATGATGATAGCGACCGGCCCTCAGGGATCCCTGAGCGATTC | | IPERFSGSNSGNTATLTISRVEAGDEAD | | | |
| | TCTGGCTCCAACTCTGGGAACACGGCCACCCTGACCATCAGCAGGGTCGAAGCCGGGGATG | | YYCQMWDSRSDHPVFGGGTKVTVL | | | |
| | AGGCCGACTATTACTGTCAAATGTGGGATAGTCGTAGTGATCATCCGGTGTTCGGCGGAGG | | | | | |
| | GACCAAGGTGACCGTCCTC | | | | | |
| ADI-75592 | GACATCCGGATGACCCAGTCTCCATCCTCCCTGTCTGCATCTATAGGAGACAGAGTCACCA | 3221 | DIRMTQSPSSLSASIGDRVTITCRASQS | 3503 | | |
| | TCACTTGCCGGGCAAGTCAGAGCATTAGTACCATTTTAAATTGGTATCAGCAGAAACCAGG | | ISTILNWYQQKPGRAPKLLIYAASSLQS | | | |
| | GAGAGCCCCTAAGCTCCTGATCTATGCTGCATCCAGTTTGCAAAGTGGGGTCCCATCAAGG | | GVPSRFSGGGSGTDFTLTISSLQPEDFA | | | |
| | TTCAGTGGCGGTGGATCTGGGACAGATTTCACTCTCACCATCAGCAGTCTGCAACCTGAAG | | TYYCQQGSTTPQTFGRGTKVEIK | | | |
| | ATTTTGCAACGTACTACTGTCAGCAGGGTTCCACTACCCCTCAGACGTTCGGCCGTGGGAC | | | | | |
| | CAAGGTGGAGATCAAA | | | | | |
| ADI-75597 | GACATCCAGATGACCCAGTCTCCATCTTCCGTGTCTGCATCTGTAGGAGACAGAGTCACCA | 3222 | DIQMTQSPSSVSASVGDRVTITCRASQG | 3504 | | |
| | TCACTTGTCGGGCGAGTCAGGGTATTAGCAGCTGGTTAGCCTGGTATCAGCAGAAACCAGG | | ISSWLAWYQQKPGKAPKLLIYAASSLQS | | | |
| | GAAAGCCCCTAAGCTCCTGATCTATGCTGCATCCAGTTTGCAAAGTGGGGTCCCATCAAGG | | GVPSRFSGSGSGTDFTLTISSLQPEDFA | | | |
| | TTCAGCGGCAGTGGATCTGGGACAGATTTCACTCTCACCATCAGCAGCCTGCAGCCTGAAG | | TYYCQQANSFPITFGGGTKVEIK | | | |
| | ATTTTGCAACTTACTATTGTCAACAGGCTAACAGTTTCCCTATCACCTTTGGCGGAGGGAC | | | | | |
| | CAAGGTGGAAATCAAA | | | | | |
| ADI-75636 | GAAATTGTGATGACCCAGTCTCCATCCTCCCTGTCTGCATCTGTAGGAGACAGAGTCACCA | 3223 | EIVMTQSPSSLSASVGDRVTITCRASHR | 3505 | | |
| | TCACTTGTCGGGCAAGTCACAGGATTGACAGGTATTTAAATTGGTATCAGCAGAAACCAGG | | IDRYLNWYQQKPGQAPKLLIYVASTLQS | | | |
| | GCAAGCCCCCAAGCTCCTGATCTATGTAGCATCCACTTTGCAAAGTGGGGTCCCATCAAGG | | GVPSRFTGSGSGTDFTLTISSLQPEDFA | | | |
| | TTCACTGGCAGTGGATCTGGGACAGATTTCACTCTCACTATCAGCAGTCTGCAACCTGAGG | | TYYCQQSYTTPRTFGPGTKVEIK | | | |
| | ATTTTGCAACTTACTACTGTCAACAGAGTTACACTACCCCTCGGACTTTCGGCCCTGGGAC | | | | | |
| | CAAGGTGGAAATCAAA | | | | | |
| ADI-75645 | GAAATTGTGATGACACAGTCTCCACTCTCCCTGCCCGTCACCCCTGGAGAGCCGGCCTCCA | 3224 | EIVMTQSPLSLPVTPGEPASISCRSSQS | 3506 | | |
| | TCTCCTGCAGGTCTAGTCAGAGCCTCTTGGATAGTGATGATGTATACACCTATTTGGACTG | | LLDSDDVYTYLDWYLQKPGQSPQLLMYT | | | |
| | GTACCTGCAGAAGCCAGGGCAGTCTCCACAGCTCCTGATGTATACACTTTCCTATCGGGCC | | LSYRASGVPDRFSGSGSGTNFTLKINRV | | | |
| | TCTGGAGTCCCAGACAGGTTCAGTGGCAGTGGGTCAGGCACTAATTTCACACTGAAAATCA | | KTEDVGVYYCMQRIEFPYTFGQGTKVEI | | | |
| | ATAGGGTAAAGACTGAGGACGTTGGAGTTTATTACTGCATGCAACGTATAGAGTTCCCCTA | | K | | | |
| | CACTTTTGGCCAGGGGACCAAGGTGGAAATCAAA | | | | | |
| ADI-75615 | CCGTCTGAGCTGACTCAGCCACCCTCAGTGTCCGTGTCCCCAGGACAGACAGCCAGCATCA | 3225 | PSELTQPPSVSVSPGQTASITCSGENLG | 3507 | | |
| | CCTGCTCTGGAGAAAATTTGGGACGTAAATATGTTTGTTGGTATCAACAGAGGTCAGGCCA | | RKYVCWYQQRSGQSPVLVIYRDDKRPSG | | | |
| | GTCCCCTGTGCTGGTTATCTATCGAGATGACAAGCGGCCCTCAGGGATCCCTGAGCGATTC | | IPERFSGSNSGNTATLTISGTQAMDEAD | | | |
| | TCTGGCTCCAATTCTGGGAACACAGCCACTCTGACCATCAGCGGGACCCAGGCTATGGATG | | YYCQAWDSRTAQVFGTGTKVTVL | | | |
| | AGGCTGACTATTACTGTCAGGCGTGGGACAGCAGGACTGCACAGGTCTTCGGAACTGGGAC | | | | | |
| | CAAGGTGACCGTCCTA | | | | | |
| ADI-75844 | GACATCCAGATGACCCAGTCTCCATCCTCCCTGTCTGCATCTGTAGGGGACAGAGTCACCA | 3226 | DIQMTQSPSSLSASVGDRVTITCRASQT | 3508 | | |
| | TCACTTGCCGGGCAAGTCAGACCATTAACAACTATTTAACTTGGTATCAGCAGAAACCAGG | | INNYLTWYQQKPGKAPNLLIYAASSLQS | | | |
| | GAAAGCCCCTAACCTCCTGATCTATGCTGCATCCAGTTTGCAAAGTGGGGTCCCATCAAGG | | GVPSRFSGSGSGTDFTLTISSLQPEDFA | | | |
| | TTCAGTGGCAGTGGATCTGGGACAGATTTCACTCTCACCATCAGCAGTCTGCAACCTGAAG | | TYYCQQSYSSPITFGQGTRLEIK | | | |
| | ATTTTGCAACTTACTACTGTCAACAGAGTTACAGTTCCCCTATCACCTTCGGCCAAGGGAC | | | | | |
| | ACGACTGGAGATTAAA | | | | | |
| ADI-75875 | AAGCCTGTGCTGACTCAGCCCCACTCTGTGTCGGAGTCTCCGGGGAAGACAGTAACCATCT | 3227 | KPVLTQPHSVSESPGKTVTISCTRSSGS | 3509 | | |
| | CCTGCACCCGCAGCAGTGGCAGCATTGCCAGCAACTATGTGCAGTGGTACCAGCAGCGCCC | | IASNYVQWYQQRPGSSPSTVIYEDNQRP | | | |
| | GGGCAGTTCACCTTCCACTGTTATCTATGAGGATAACCAAAGACCCTCTGGGGTCCCTGAT | | SGVPDRFSGSIDSSSNSASLTISGLQTD | | | |
| | CGGTTCTCTGGCTCCATCGACAGCTCCTCCAACTCTGCCTCCCTCACCATCTCTGGACTGC | | DEADYYCQSYDSSLWVFGGGTKLTVL | | | |
| | AGACTGACGACGAGGCTGACTACTACTGTCAGTCTTATGATAGCAGCCTTTGGGTGTTCGG | | | | | |
| | CGGAGGGACCAAGCTCACCGTCTTA | | | | | |
| ADI-75714 | CAGTCTGTGTTGACGCAGCCGCCCTCAGTGTCTGGGGCCCCAGGGCAGAGGGTCACCATCT | 3228 | QSVLTQPPSVSGAPGQRVTISCTGSNSN | 3510 | | |
| | CCTGCACTGGGAGCAACTCCAACATCGGGGCAGGTTATGATGTACACTGGTACCAGCAACT | | IGAGYDVHWYQQLPGTAPKLLIYGNNNR | | | |
| | TCCAGGAACAGCCCCCAAACTCCTCATCTATGGTAACAACAATCGGCCCTCAGGGGTCCCT | | PSGVPDRFSGSKSVTSASLAITGLQAED | | | |
| | GACCGATTCTCTGGCTCCAAGTCTGTCACCTCAGCCTCCCTGGCCATCACTGGGCTCCAGG | | EADYYCQSYDSSLSDSVFGGGTKVTVL | | | |
| | CTGAGGATGAGGCTGATTATTACTGCCAGTCCTATGACAGCAGCCTGAGTGATTCGGTGTT | | | | | |
| | CGGCGGAGGGACCAAGGTGACCGTCCTA | | | | | |
| ADI-75688 | GAAATTGTGATGACCCAGTCTCCAGGCACCCTGTCTTTGTCTCCAGGGGAAAGAGCCACCC | 3229 | EIVMTQSPGTLSLSPGERATLSCRASQS | 3511 | | |
| | TCTCCTGCAGGGCCAGTCAGAGTGTTAGCAGCAGCTACTTAGCCTGGTACCAGCAGAAACC | | VSSSYLAWYQQKPGQAPRLLIYGASSRA | | | |
| | TGGCCAGGCTCCCAGGCTCCTCATCTATGGTGCATCCAGCAGGGCCACTGGCATCCCAGAC | | TGIPDRFSGSGSGTDFTLTISRLEPEDF | | | |
| | AGGTTCAGTGGCAGTGGGTCTGGGACAGACTTCACTCTCACCATCAGCAGACTGGAGCCTG | | AVYYCQQYGSSPGTFGQGTKVEIK | | | |
| | AAGATTTTGCAGTGTATTACTGTCAGCAGTATGGTAGCTCACCTGGAACTTTTGGCCAGGG | | | | | |
| | GACCAAGGTGGAGATCAAA | | | | | |
| ADI-75637 | GACATCACGATGACGCAGTCTCCATCCTCCCTGTCTGCATCTGTAGGAGACAGAGTCACCA | 3230 | DITMTQSPSSLSASVGDRVTIACRASQG | 3512 | | |
| | TCGCTTGCCGGGCAAGTCAGGGCATTAGCACCTCTTTAAATTGGTGTCAGCAGAAACCAGG | | ISTSLNWCQQKPGKAPKLLISAASSLQS | | | |
| | GAAAGCCCCTAAGCTCCTGATCTCTGCTGCATCCAGTTTGCAAAGTGGGGTCCCATCAAGG | | GVPSRFSGSGSGTDFTLTISNLQPEDFA | | | |
| | TTCAGTGGCAGTGGATCTGGGACAGATTTCACTCTCACCATCAGCAATCTGCAACCTGAAG | | TYYCQQSYSTPWTFGQGTKVEIK | | | |
| | ATTTTGCAACTTACTACTGTCAACAGAGTTATAGTACCCCGTGGACGTTCGGCCAAGGGAC | | | | | |
| | CAAGGTGGAAATCAAA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| ADI-75745 | IGKV1D- | EIVMTQ | 1891 | RASQSIG | 2249 | WYQQKS | 2312 | ATSSLH | 2523 | GVPSRFSG | 2696 | QQSYN | 2924 | FGQGTK | 2937 |
| | 39 | SPSSLS | | TYLN | | GKAPKL | | S | | SGYGTDFT | | SPGFS | | VEIK | |
| | | ASVGDR | | | | LIY | | | | LTIDSVQP | | | | | |
| | | VTITC | | | | | | | | EDFATYYC | | | | | |
| ADI-75665 | IGLV1-40 | QSVVTQ | 1972 | TGSSSNI | 2250 | WYQQLP | 2314 | GDNNRP | 2524 | GVPDRFSG | 2697 | QSYDS | 2925 | FGGGTK | 2939 |
| | | PPSVSG | | GTGYDVH | | GTAPKL | | S | | SKSGTSAS | | TLIGP | | LTVL | |
| | | APGQRV | | | | LIY | | | | LAITGLQP | | V | | | |
| | | TISC | | | | | | | | DDEAAYYC | | | | | |
| ADI-75812 | IGLV6-57 | NFMLTQ | 1992 | TRSSGYI | 2251 | WYQQRP | 2380 | EDNQRP | 2431 | GVPERFSA | 2698 | QSYES | 2926 | FGGGTK | 2939 |
| | | PHSVSE | | ASNYVL | | GSAPTI | | S | | SVDSSSNS | | DTVI | | LTVL | |
| | | SPGKTV | | | | VIS | | | | ASLTISGL | | | | | |
| | | TISC | | | | | | | | KTEDEADY | | | | | |
| | | | | | | | | | | YC | | | | | |
| ADI-75788 | IGLV3-21 | SPVLTQ | 2040 | GGDNIGS | 2252 | WYQQKP | 2283 | DDTDRP | 2419 | GIPERFSG | 2545 | QVWDT | 2927 | FGTGTK | 2949 |
| | | PPSVSV | | KNVH | | GQAPVL | | S | | SNSGNTAT | | SGDHY | | LTVL | |
| | | APGQTA | | | | VVY | | | | LTISRVEA | | V | | | |
| | | RITC | | | | | | | | GDEADYYC | | | | | |
| ADI-75599 | IGLV1-47 | PSELTQ | 2041 | SGSSSNI | 2253 | WYQQLP | 2314 | SNNQRP | 2463 | GVPDRFSG | 2669 | AAWDD | 2928 | FGGGTK | 2939 |
| | | PPSASG | | GSNSVY | | GTAPKL | | S | | SKSGTSAS | | SRWV | | LTVL | |
| | | TPGQGV | | | | LIY | | | | LAISGLRS | | | | | |
| | | TISC | | | | | | | | EDEADYYC | | | | | |
| ADI-75664 | IGKV3-20 | EIAMTQ | 2042 | RASQSVS | 2230 | WYQQKP | 2258 | GASSRA | 2464 | GIPDRFSG | 2616 | QQYGS | 2929 | FGQGTK | 2937 |
| | | SPGTLS | | SSYLA | | GQAPRL | | T | | SGSGTDFT | | SPYT | | VEIK | |
| | | LSPGER | | | | LIY | | | | LTISRLEP | | | | | |
| | | ATLSC | | | | | | | | EDFAVYYC | | | | | |
| ADI-75646 | IGKV4-1 | DIQMTQ | 1927 | KSSQSVL | 2254 | WYQQKP | 2290 | WASTRE | 2422 | GVPDRFSG | 2619 | QQYYT | 2930 | FGPGTK | 2942 |
| | | SPDSLA | | YSSNNKN | | GQPPKL | | S | | SGSGTDFT | | PPLT | | VEIK | |
| | | VSLGER | | YLA | | LIY | | | | LTISSLQA | | | | | |
| | | ATINC | | | | | | | | EDVAVYYC | | | | | |
| ADI-75723 | IGLV6-57 | TFMLTQ | 2043 | TRSSGSI | 2109 | WYQQRP | 2298 | QDNERP | 2525 | GVPDRFSG | 2574 | QSYDT | 2931 | FGGGTK | 2939 |
| | | PHSVSE | | ASNYVQ | | GSAPTT | | S | | SIDSSSNS | | SNEV | | LTVL | |
| | | SPGKTV | | | | VIY | | | | ASLTISGL | | | | | |
| | | TISC | | | | | | | | KTEDEADY | | | | | |
| | | | | | | | | | | YC | | | | | |
| ADI-75595 | IGLV1-47 | QSVLTQ | 2044 | SGRSSNI | 2255 | WYQQFP | 2324 | GDDQRP | 2526 | GVPDRFSG | 2669 | ATWDD | 2932 | FGGGTK | 2939 |
| | | PPSASG | | GSNSVN | | GTAPKL | | S | | SKSGTSAS | | SGWV | | LTVL | |
| | | TPGQRV | | | | LIY | | | | LAISGLRS | | | | | |
| | | TITC | | | | | | | | EDEADYYC | | | | | |
| ADI-75824 | IGLV1-44 | SHELTQ | 2045 | SGSTSNI | 2214 | WYQHLP | 2307 | SDDQRP | 2527 | GVPDRFSG | 2699 | AAWDD | 2933 | FGGGTK | 2939 |
| | | PPSASG | | GSNTVK | | GTAPKL | | S | | SKSGTSAS | | TLAGP | | LTVL | |
| | | TPGQRV | | | | LIY | | | | LAISGLQS | | V | | | |
| | | TISC | | | | | | | | EDESDYYC | | | | | |
| ADI-75749 | IGKV4-1 | DIVMTQ | 2046 | KSSQSVL | 2256 | WYQQRP | 2381 | WASTRE | 2422 | GVPDRFSG | 2619 | QQYYS | 2934 | FGPGTK | 2950 |
| | | SPDSLA | | YRSKNKN | | GQPPKL | | S | | SGSGTDFT | | SPS | | VDIK | |
| | | VSLGER | | YLA | | LIY | | | | LTISSLQA | | | | | |
| | | ATINC | | | | | | | | EDVAVYYC | | | | | |
| ADI-75867 | IGKV1D- | DIQMTQ | 1896 | RASQSIS | 2066 | WYQQKP | 2316 | AASSLP | 2509 | GVPSRFRG | 2626 | QQSYN | 2935 | FGQGTK | 2937 |
| | 39 | SPSSLS | | TYLN | | GKAPKV | | S | | SGSGTDFT | | APRT | | VEIK | |
| | | ASVGDR | | | | LIY | | | | LTISSLQP | | | | | |
| | | VTIAC | | | | | | | | EDFATYYC | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| ADI-75745 | GAAATTGTGATGACGCAGTCTCCATCCTCCCTGTCTGCATCTGTAGGAGACAGAGTCACCA | 3231 | EIVMTQSPSSLSASVGDRVTITCRASQS | 3513 | | |
| | TCACTTGCCGGGCAAGTCAGAGCATTGGCACCTATTTAAATTGGTATCAGCAGAAATCAGG | | IGTYLNWYQQKSGKAPKLLIYATSSLHS | | | |
| | GAAAGCCCCTAAACTCCTCATCTATGCTACATCCAGTTTGCATAGTGGTGTCCCATCAAGG | | GVPSRFSGSGYGTDFTLTIDSVQPEDFA | | | |
| | TTCAGTGGCAGTGGATATGGGACAGATTTCACTCTCACCATCGACAGTGTGCAACCTGAAG | | TYYCQQSYNSPGFSFGQGTKVEIK | | | |
| | ATTTTGCAACTTATTACTGTCAACAGAGTTACAATAGCCCTGGGTTCAGTTTTGGCCAGGG | | | | | |
| | GACCAAGGTGGAAATCAAA | | | | | |
| ADI-75665 | CAGTCTGTCGTGACGCAGCCGCCCTCAGTGTCTGGGGCCCCAGGGCAGAGGGTCACCATCT | 3232 | QSVVTQPPSVSGAPGQRVTISCTGSSSN | 3514 | | |
| | CCTGCACTGGGAGCAGCTCCAACATCGGGACAGGTTATGATGTACACTGGTACCAACAACT | | IGTGYDVHWYQQLPGTAPKLLIYGDNNR | | | |
| | TCCAGGAACAGCCCCCAAACTCCTCATCTATGGTGACAACAATCGGCCCTCAGGAGTCCCT | | PSGVPDRFSGSKSGTSASLAITGLQPDD | | | |
| | GACCGATTCTCTGGCTCCAAGTCTGGCACCTCAGCCTCCCTGGCCATCACTGGGCTCCAGC | | EAAYYCQSYDSTLIGPVFGGGTKLTVL | | | |
| | CTGACGATGAGGCTGCTTATTACTGCCAGTCCTATGACAGCACCCTGATTGGTCCGGTATT | | | | | |
| | CGGCGGAGGGACCAAGCTCACCGTCCTA | | | | | |
| ADI-75812 | AATTTTATGCTGACTCAGCCCCACTCTGTGTCGGAGTCTCCGGGGAAGACGGTAACCATCT | 3233 | NFMLTQPHSVSESPGKTVTISCTRSSGY | 3515 | | |
| | CCTGCACCCGCAGCAGTGGCTACATTGCCAGCAACTATGTGCTATGGTACCAACAGCGCCC | | IASNYVLWYQQRPGSAPTIVISEDNQRP | | | |
| | GGGCAGTGCCCCCACCATTGTGATCTCTGAGGATAACCAAAGACCCTCTGGGGTCCCTGAG | | SGVPERFSASVDSSSNSASLTISGLKTE | | | |
| | CGGTTCTCTGCCTCCGTCGACAGCTCCTCCAACTCTGCCTCCCTCACCATCTCTGGACTGA | | DEADYYCQSYESDTVIFGGGTKLTVL | | | |
| | AGACTGAGGACGAGGCTGACTACTACTGTCAGTCTTATGAAAGTGACACTGTGATTTTCGG | | | | | |
| | CGGAGGCACCAAGCTCACCGTCCTA | | | | | |
| ADI-75788 | TCGCCTGTGCTGACTCAGCCACCCTCGGTGTCAGTGGCCCCTGGGCAGACGGCCAGGATCA | 3234 | SPVLTQPPSVSVAPGQTARITCGGDNIG | 3516 | | |
| | CCTGTGGGGGAGACAACATTGGAAGTAAAAATGTCCACTGGTACCAGCAGAAGCCAGGCCA | | SKNVHWYQQKPGQAPVLVVYDDTDRPSG | | | |
| | GGCCCCTGTGCTGGTCGTCTATGATGATACCGACCGGCCCTCAGGGATCCCTGAGCGATTC | | IPERFSGSNSGNTATLTISRVEAGDEAD | | | |
| | TCTGGCTCCAATTCTGGGAACACGGCCACCCTGACCATCAGCAGGGTCGAAGCCGGGGATG | | YYCQVWDTSGDHYVFGTGTKLTVL | | | |
| | AGGCCGACTATTACTGTCAGGTGTGGGATACTAGTGGTGATCATTATGTCTTCGGAACTGG | | | | | |
| | GACCAAGCTCACCGTCCTA | | | | | |
| ADI-75599 | CCGTCTGAGCTGACTCAGCCACCCTCAGCGTCTGGGACCCCCGGGCAGGGGGTCACCATCT | 3235 | PSELTQPPSASGTPGQGVTISCSGSSSN | 3517 | | |
| | CTTGCTCTGGAAGCAGCTCCAACATCGGAAGTAATTCTGTATACTGGTACCAGCAGCTCCC | | IGSNSVYWYQQLPGTAPKLLIYSNNQRP | | | |
| | AGGAACGGCCCCCAAACTCCTCATCTATAGTAATAATCAGCGGCCCTCAGGGGTCCCTGAC | | SGVPDRFSGSKSGTSASLAISGLRSEDE | | | |
| | CGATTCTCTGGCTCCAAGTCTGGCACCTCAGCCTCCCTGGCCATCAGTGGGCTCCGGTCCG | | ADYYCAAWDDSRWVFGGGTKLTVL | | | |
| | AGGATGAGGCTGATTATTACTGTGCAGCATGGGATGACAGCCGTTGGGTGTTCGGCGGAGG | | | | | |
| | GACCAAGCTCACCGTCCTA | | | | | |
| ADI-75664 | GAAATTGCAATGACGCAGTCTCCAGGCACCCTGTCTTTGTCTCCAGGGGAAAGAGCCACCC | 3236 | EIAMTQSPGTLSLSPGERATLSCRASQS | 3518 | | |
| | TCTCCTGCAGGGCCAGTCAGAGTGTTAGCAGCAGCTACTTAGCCTGGTACCAGCAGAAACC | | VSSSYLAWYQQKPGQAPRLLIYGASSRA | | | |
| | TGGCCAGGCTCCCAGGCTCCTCATCTATGGTGCATCCAGCAGGGCCACTGGCATCCCAGAC | | TGIPDRFSGSGSGTDFTLTISRLEPEDF | | | |
| | AGGTTCAGTGGCAGTGGGTCTGGGACAGACTTCACTCTCACCATCAGCAGACTGGAGCCTG | | AVYYCQQYGSSPYTFGQGTKVEIK | | | |
| | AAGATTTTGCAGTGTATTACTGTCAGCAGTATGGTAGCTCACCGTACACTTTTGGCCAGGG | | | | | |
| | GACCAAGGTGGAAATCAAA | | | | | |
| ADI-75646 | GACATCCAGATGACCCAGTCTCCAGACTCCCTGGCTGTGTCTCTGGGCGAGAGGGCCACCA | 3237 | DIQMTQSPDSLAVSLGERATINCKSSQS | 3519 | | |
| | TCAACTGCAAGTCCAGCCAGAGTGTTTTATACAGCTCCAACAATAAGAACTACTTAGCTTG | | VLYSSNNKNYLAWYQQKPGQPPKLLIYW | | | |
| | GTACCAGCAGAAACCAGGACAGCCTCCTAAGCTGCTCATTTACTGGGCATCTACCCGGGAA | | ASTRESGVPDRFSGSGSGTDFTLTISSL | | | |
| | TCCGGGGTCCCTGACCGATTCAGTGGCAGCGGGTCTGGGACAGATTTCACTCTCACCATCA | | QAEDVAVYYCQQYYTPPLTFGPGTKVEI | | | |
| | GCAGCCTGCAGGCTGAAGATGTGGCAGTTTATTACTGTCAGCAATATTATACTCCTCCTCT | | K | | | |
| | CACTTTCGGCCCTGGGACCAAGGTGGAAATCAAA | | | | | |
| ADI-75723 | ACTTTTATGCTGACTCAGCCCCACTCTGTGTCGGAGTCTCCGGGGAAGACGGTAACCATCT | 3238 | TFMLTQPHSVSESPGKTVTISCTRSSGS | 3520 | | |
| | CCTGCACCCGCAGCAGTGGCAGCATTGCCAGCAACTATGTGCAGTGGTACCAGCAGCGCCC | | IASNYVQWYQQRPGSAPTTVIYQDNERP | | | |
| | GGGCAGTGCCCCCACCACTGTGATCTATCAGGATAATGAAAGACCCTCTGGAGTCCCTGAT | | SGVPDRFSGSIDSSSNSASLTISGLKTE | | | |
| | CGGTTCTCTGGCTCCATCGACAGTTCTTCCAACTCTGCCTCCCTCACCATCTCTGGACTGA | | DEADYYCQSYDTSNEVFGGGTKLTVL | | | |
| | AGACTGAGGACGAGGCTGACTACTACTGTCAGTCTTATGATACTAGCAATGAGGTGTTCGG | | | | | |
| | CGGAGGGACCAAGCTCACCGTCCTA | | | | | |
| ADI-75595 | CAGTCTGTGCTGACTCAGCCACCCTCAGCGTCTGGGACCCCCGGGCAGAGGGTCACCATCA | 3239 | QSVLTQPPSASGTPGQRVTITCSGRSSN | 3521 | | |
| | CTTGTTCTGGAAGGAGCTCCAACATTGGAAGTAATTCTGTAAACTGGTATCAACAGTTCCC | | IGSNSVNWYQQFPGTAPKLLIYGDDQRP | | | |
| | AGGAACGGCCCCCAAACTCCTCATCTATGGTGATGATCAACGGCCCTCAGGGGTCCCGGAC | | SGVPDRFSGSKSGTSASLAISGLRSEDE | | | |
| | CGATTTTCTGGCTCCAAGTCTGGCACCTCAGCCTCCCTGGCCATCAGTGGGCTCCGGTCCG | | ADYYCATWDDSGWVFGGGTKLTVL | | | |
| | AAGATGAGGCTGATTATTATTGTGCAACATGGGATGACAGCGGTTGGGTGTTCGGCGGAGG | | | | | |
| | GACCAAGCTCACCGTCCTA | | | | | |
| ADI-75824 | TCGCATGAGCTGACTCAGCCACCCTCAGCGTCTGGGACCCCCGGGCAGAGGGTCACCATCT | 3240 | SHELTQPPSASGTPGQRVTISCSGSTSN | 3522 | | |
| | CTTGTTCTGGAAGCACCTCCAACATCGGGAGTAATACTGTAAAATGGTACCAGCACCTCCC | | IGSNTVKWYQHLPGTAPKLLIYSDDQRP | | | |
| | AGGAACGGCCCCCAAACTCCTCATCTATAGTGATGATCAGCGGCCCTCAGGGGTCCCTGAC | | SGVPDRFSGSKSGTSASLAISGLQSEDE | | | |
| | CGATTCTCTGGCTCCAAGTCTGGCACCTCAGCCTCCCTGGCCATCAGTGGGCTCCAGTCTG | | SDYYCAAWDDTLAGPVFGGGTKLTVL | | | |
| | AGGATGAGTCTGATTATTACTGTGCAGCATGGGATGACACTTTGGCTGGTCCGGTGTTCGG | | | | | |
| | CGGAGGGACCAAGCTCACCGTCCTA | | | | | |
| ADI-75749 | GATATTGTGATGACCCAGTCTCCAGACTCCCTGGCTGTGTCTCTGGGCGAGAGGGCCACCA | 3241 | DIVMTQSPDSLAVSLGERATINCKSSQS | 3523 | | |
| | TCAACTGCAAGTCCAGCCAGAGTGTTTTATACAGGTCCAAAAATAAGAACTACTTAGCTTG | | VLYRSKNKNYLAWYQQRPGQPPKLLIYW | | | |
| | GTACCAGCAGAGACCAGGACAGCCTCCTAAGCTGCTCATTTACTGGGCATCTACCCGGGAA | | ASTRESGVPDRFSGSGSGTDFTLTISSL | | | |
| | TCCGGGGTCCCTGACCGATTCAGTGGCAGCGGGTCTGGGACAGATTTCACTCTCACCATCA | | QAEDVAVYYCQQYYSSPSFGPGTKVDIK | | | |
| | GCAGCCTGCAGGCTGAAGATGTGGCAGTTTATTACTGTCAGCAATATTATAGTTCTCCCTC | | | | | |
| | TTTCGGCCCTGGGACCAAAGTGGATATCAAA | | | | | |
| ADI-75867 | GACATCCAGATGACCCAGTCTCCATCTTCCCTGTCTGCATCTGTAGGAGACAGAGTCACCA | 3242 | DIQMTQSPSSLSASVGDRVTIACRASQS | 3524 | | |
| | TCGCTTGCCGGGCAAGTCAGAGCATTAGCACCTATTTAAATTGGTATCAGCAGAAACCAGG | | ISTYLNWYQQKPGKAPKVLIYAASSLPS | | | |
| | GAAAGCCCCTAAGGTCCTGATCTATGCTGCATCCAGTTTACCAAGTGGGGTCCCATCAAGG | | GVPSRFRGSGSGTDFTLTISSLQPEDFA | | | |
| | TTCCGTGGCAGTGGATCTGGGACAGATTTCACTCTCACCATCAGCAGTCTGCAACCTGAAG | | TYYCQQSYNAPRTFGQGTKVEIK | | | |
| | ATTTTGCAACTTACTACTGTCAACAGAGTTACAATGCCCCTCGGACGTTCGGCCAAGGGAC | | | | | |
| | CAAGGTGGAAATCAAA | | | | | |

| | | | SEQ | | SEQ | | SEQ | | SEQ | | SEQ | | SEQ | | SEQ |
|---|---|---|---|---|---|---|---|---|---|---|---|---|---|---|---|
| | VL | VL | ID | VL | ID | VL | ID | VL | ID | VL | ID | VL | ID | VL | ID |
| Clone ID | Germline | FR1 | NO: | CDR1 | NO: | FR2 | NO: | CDR2 | NO: | FR3 | NO: | CDR3 | NO: | FR4 | NO: |
| VYD224 | IGKV3-11 | EIVLTQ | 1945 | RASQSVS | 2119 | WYQQRP | 2306 | DASNRA | 2448 | GIPARFSG | 2596 | QQRSN | 2794 | FGGGTK | 2936 |
| (ADI- | | SPATLS | | TYLA | | GQAPRL | | T | | SGSETDFT | | WLT | | VEIK | |
| 80707) | | LSPGER | | | | LIY | | | | LTISSLEP | | | | | |
| | | ATLSC | | | | | | | | EDFAVYYC | | | | | |
| VYD225 | | DIQMT | 2047 | ITNSLA | 2067 | WYQQ | 2267 | AASTL | 2398 | GVPPRF | 2544 | QQYF | 2718 | FGQGT | 2937 |
| | | QSPS | | RASQG | | KPGK | | ES | | RGSGSG | | DSPH | | KVEIK | |
| | | SLSAS | | | | APNLL | | | | TDFTLTI | | T | | | |
| | | VGDT | | | | LY | | | | SSLQPE | | | | | |
| | | VTITC | | | | | | | | DFATFY | | | | | |
| | | | | | | | | | | C | | | | | |

| | | SEQ | | SEQ | Reverted | SEQ |
|---|---|---|---|---|---|---|
| | | ID | | ID | VL | ID |
| Clone ID | VL DNA | NO: | VL Protein | NO: | Protein | NO: |
| VYD224 | GAAATTGTGTTGACACAGTCTCCAGCCACCCTGTCTTTGTCTCCAGGGGAAAGAGCCACCC | 3243 | EIVLTQSPATLSLSPGERATLSCRASQS | 3353 | EIVLTQSPA | 3353 |
| (ADI- | TCTCCTGCAGGGCCAGTCAGAGTGTTAGCACCTACTTAGCCTGGTACCAACAGAGACCTGG | | VSTYLAWYQQRPGQAPRLLIYDASNRAT | | TLSLSPGER | |
| 80707) | CCAGGCTCCCAGGCTCCTCATCTATGATGCATCCAACAGGGCCACTGGCATCCCAGCCAGG | | GIPARFSGSGSETDFTLTISSLEPEDFA | | ATLSCRASQ | |
| | TTCAGTGGCAGTGGGTCTGAGACAGACTTCACTCTCACCATCAGCAGCCTGGAGCCTGAAG | | VYYCQQRSNWLTFGGGTKVEIK | | SVSTYLAWY | |
| | ATTTTGCAGTTTATTATTGTCAACAGCGGAGCAACTGGCTCACTTTCGGCGGAGGGACCAA | | | | QQRPGQAPR | |
| | GGTGGAGATCAAA | | | | LLIYDASNR | |
| VYD225 | GACATTCAGATGACTCAGTCGCCTTCATCTCTGTCTGCATCTGTCGGC | 3244 | DIQMTQSPSSLSASVGDTVTITCR | 3525 | | |
| | GATACTGTTACTATCACATGTAGAGCCAGCCAGGGAATTACGAATTCAC | | ASQGITNSLAWYQQKPGKAPNLL | | | |
| | TTGCTTGGTATCAGCAAAAACCTGGAAAAGCACCTAACCTCTTGCTCTA | | LYAASTLESGVPPRFRGSGSGTD | | | |
| | CGCGGCGTCCACCTTGGAATCTGGTGTCCCACCACGATTTCGGGGCA | | FTLTISSLQPEDFATFYCQQYFDS | | | |
| | GTGGAAGTGGCACAGACTTTACTCTCACCATCTCGAGCTTGCAACCTG | | PHTFGQGTKVEIK | | | |
| | AGGATTTCGCTACATTTTACTGTCAACAATATTTCGACTCTCCCCACAC | | | | | |
| | CTTCGGCCAAGGCACAAAGGTGGAGATTAAA | | | | | |

TABLE 5

| | VH | VH | | SEQ | | SEQ | | SEQ | | SEQ | | SEQ | | SEQ | |
|---|---|---|---|---|---|---|---|---|---|---|---|---|---|---|---|
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81604 | 62 | IGHV5- | QVQLV | 174 | YSFTSY | 372 | WVRQMP | 476 | IIYPGD | 635 | QVTISA | 1010 | ARGEGYG | 6474 | WGQGTM |
| | | 51 | QSGAE | | WIG | | GKGLEW | | SDTRYS | | DKSIST | | DYADAFD | | VTVSS |
| | | | VKKPG | | | | MG | | PSFQG | | AYLQWS | | I | | |
| | | | ESLKI | | | | | | | | SLKASD | | | | |
| | | | SCKGS | | | | | | | | TAMYYC | | | | |
| | | | G | | | | | | | | | | | | |
| ADI-81710 | 80 | IGHV5- | QVQLV | 5313 | YSFTTY | 5515 | WVRQMP | 476 | IIYPGD | 635 | QVTISA | 6174 | ARRIRTY | 6475 | WGQGTL |
| | | 51 | QSGAE | | WIG | | GKGLEW | | SDTRYS | | DKSMTT | | PPCFDY | | VTVSS |
| | | | VKKPG | | | | MG | | PSFQG | | AYLQWS | | | | |
| | | | ESLKI | | | | | | | | SLKASD | | | | |
| | | | SCKIS | | | | | | | | TAMYFC | | | | |
| | | | G | | | | | | | | | | | | |
| ADI-81579 | 40 | IGHV4- | QVQLQ | 5314 | ASVSGE | 5516 | WIRQPP | 5773 | YFYSTG | 5869 | RVTISV | 6175 | ARADRRL | 6476 | WGQGTT |
| | | 61 | QSGPG | | GNYWT | | GKRLES | | KTNYNP | | DKSKNL | | PNDAFDV | | VTVSS |
| | | | LVKPS | | | | IA | | SLKS | | FSLRLD | | | | |
| | | | ETLSL | | | | | | | | SVTAAD | | | | |
| | | | TCSVS | | | | | | | | TALYYC | | | | |
| | | | G | | | | | | | | | | | | |
| ADI-81656 | 29 | IGHV1- | QVQLV | 50 | GTFSSY | 5517 | WVRQVP | 511 | RVIPFL | 5870 | RLTIAV | 6176 | AEHYHES | 6477 | WGQGTM |
| | | 69 | QSGAE | | GIS | | GQGLEW | | RILDYA | | DTSTST | | RFGGGIS | | VTVSS |
| | | | VKKPG | | | | MG | | PKFQG | | AYLELS | | DDAFNI | | |
| | | | SSVKV | | | | | | | | SLRSED | | | | |
| | | | SCKAS | | | | | | | | TAVYYC | | | | |
| | | | G | | | | | | | | | | | | |
| ADI-81640 | 13 | IGHV3- | QVQLL | 5315 | LIISDH | 5518 | WVRQAP | 432 | RSANRV | 5871 | RFTISR | 6177 | VVIAGTY | 6478 | WGQGTL |
| | | 72 | ESGGG | | YID | | GKGLEW | | NSYTAL | | DDSKNT | | GS | | VTVSS |
| | | | LVQPG | | | | VG | | YAASVM | | LFLQMT | | | | |
| | | | GSLRL | | | | | | G | | SLKTDD | | | | |
| | | | SCAIS | | | | | | | | TAVYHC | | | | |
| | | | G | | | | | | | | | | | | |
| ADI-81586 | 47 | IGHV4- | QVQLQ | 5316 | GSVNGD | 5519 | WIRQPP | 5774 | EISHDG | 5872 | RVSISL | 6178 | ARGLSAV | 6479 | WGQGTL |
| | | 34 | QWGAG | | YWS | | GKGLEL | | TTKYKS | | DTSKNQ | | VTVSGWF | | VTVSS |
| | | | LLKPS | | | | IG | | ALES | | FSLKLS | | DS | | |
| | | | ETLSL | | | | | | | | SVTAAD | | | | |
| | | | TCAFS | | | | | | | | TAMYYC | | | | |
| | | | G | | | | | | | | | | | | |
| ADI-81663 | 35 | IGHV3- | EVQLQ | 5317 | FTFSDH | 5520 | WVRQAP | 432 | RTRNKA | 5873 | RFTISR | 6179 | ARVGAAL | 6480 | WGQGTL |
| | | 72 | ESGGG | | FMD | | GKGLEW | | NSYTTE | | DDSKNS | | GGVCDY | | VTVSS |
| | | | LVQPG | | | | VG | | YAASVK | | LYLQMN | | | | |
| | | | GSLRL | | | | | | G | | SLKTED | | | | |
| | | | SCAAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81604 | 1299 | CAGGTCCAGCTGGTGCAGTCTGGAGCAGAGGTGAAAAAGCC | | | | | | 6832 | QVQLVQSGAEVKKP | | 7185 | 0 | | 0 | |
| | | CGGGGAGTCTCTGAAGATCTCCTGTAAGGGTTCTGGATACA | | | | | | | GESLKISCKGSGYS | | | | | | |
| | | GCTTTACCAGCTACTGGATCGGCTGGGTGCGCCAGATGCCC | | | | | | | FTSYWIGWVRQMPG | | | | | | |
| | | GGGAAAGGCCTGGAGTGGATGGGGATCATCTATCCTGGTGA | | | | | | | KGLEWMGIIYPGDS | | | | | | |
| | | CTCTGATACCAGATACAGCCCGTCCTTCCAAGGCCAGGTCA | | | | | | | DTRYSPSFQGQVTI | | | | | | |
| | | CCATCTCAGCCGACAAGTCCATCAGCACCGCCTACCTGCAG | | | | | | | SADKSISTAYLQWS | | | | | | |
| | | TGGAGCAGCCTGAAGGCCTCGGACACCGCCATGTATTACTG | | | | | | | SLKASDTAMYYCAR | | | | | | |
| | | TGCGAGGGGGGAAGGGTACGGTGACTACGCAGATGCTTTTG | | | | | | | GEGYGDYADAFDIW | | | | | | |
| | | ATATCTGGGGCCAAGGGACAATGGTCACCGTCTCCTCA | | | | | | | GQGTMVTVSS | | | | | | |
| ADI-81710 | 1286 | CAGGTCCAGCTGGTGCAGTCTGGAGCAGAGGTGAAAAAGCC | | | | | | 6833 | QVQLVQSGAEVKKP | | 7186 | 7 | | 5 | |
| | | CGGGGAGTCTCTGAAGATCTCCTGTAAAATCTCTGGATACA | | | | | | | GESLKISCKISGYS | | | | | | |
| | | GCTTTACCACCTACTGGATCGGCTGGGTGCGCCAGATGCCC | | | | | | | FTTYWIGWVRQMPG | | | | | | |
| | | GGGAAAGGCCTGGAGTGGATGGGCATCATCTATCCTGGTGA | | | | | | | KGLEWMGIIYPGDS | | | | | | |
| | | CTCTGATACCAGATACAGCCCGTCCTTCCAAGGCCAGGTCA | | | | | | | DTRYSPSFQGQVTI | | | | | | |
| | | CCATCTCAGCCGACAAGTCCATGACCACCGCCTACCTGCAG | | | | | | | SADKSMTTAYLQWS | | | | | | |
| | | TGGAGCAGCCTGAAGGCCTCGGACACCGCCATGTATTTCTG | | | | | | | SLKASDTAMYFCAR | | | | | | |
| | | TGCGAGACGAATTAGGACCTACCCCCCCTGCTTTGACTACT | | | | | | | RIRTYPPCFDYWGQ | | | | | | |
| | | GGGGCCAGGGAACCCTGGTCACCGTCTCCTCA | | | | | | | GTLVTVSS | | | | | | |
| ADI-81579 | 1287 | CAGGTACAGCTGCAGCAGTCGGGCCCAGGGCTGGTGAAGCC | | | | | | 6834 | QVQLQQSGPGLVKP | | 7187 | 27 | | 18 | |
| | | TTCGGAGACCCTGTCCCTCACGTGCTCTGTCTCTGGTGCCT | | | | | | | SETLSLTCSVSGAS | | | | | | |
| | | CCGTCAGCGGTGAGGGTAACTACTGGACCTGGATCCGGCAG | | | | | | | VSGEGNYWTWIRQP | | | | | | |
| | | CCCCCAGGGAAACGACTGGAGTCCATTGCCTATTTCTATTC | | | | | | | PGKRLESIAYFYST | | | | | | |
| | | TACTGGGAAAACCAACTACAACCCCTCCCTCAAGAGTCGAG | | | | | | | GKTNYNPSLKSRVT | | | | | | |
| | | TCACCATATCTGTGGACAAGTCCAAAAATCTTTTCTCCCTG | | | | | | | ISVDKSKNLFSLRL | | | | | | |
| | | AGGCTGGATTCTGTGACTGCTGCGGACACGGCCCTTTATTA | | | | | | | DSVTAADTALYYCA | | | | | | |
| | | CTGTGCCAGAGCGGACCGCCGACTACCGAATGATGCTTTTG | | | | | | | RADRRLPNDAFDVW | | | | | | |
| | | ATGTTTGGGGCCAAGGGACCACGGTCACCGTCTCCTCA | | | | | | | GQGTTVTVSS | | | | | | |
| ADI-81656 | 1299 | CAGGTCCAGCTGGTGCAGTCTGGGGCTGAGGTGAAGAAGCC | | | | | | 6835 | QVQLVQSGAEVKKP | | 7188 | 16 | | 13 | |
| | | TGGGTCCTCGGTGAAGGTCTCCTGCAAGGCTTCTGGAGGCA | | | | | | | GSSVKVSCKASGGT | | | | | | |
| | | CCTTCAGCAGCTATGGCATTAGCTGGGTGCGACAGGTCCCT | | | | | | | FSSYGISWVRQVPG | | | | | | |
| | | GGACAAGGGCTTGAGTGGATGGGAAGGGTCATCCCCTTCCT | | | | | | | QGLEWMGRVIPFLR | | | | | | |
| | | TCGTATACTTGACTATGCACCGAAGTTCCAGGGCAGACTCA | | | | | | | ILDYAPKFQGRLTI | | | | | | |
| | | CGATTGCCGTGGACACATCCACGAGCACAGCCTACCTGGAA | | | | | | | AVDTSTSTAYLELS | | | | | | |
| | | CTGAGCAGCCTCAGATCTGAAGACACGGCCGTGTATTATTG | | | | | | | SLRSEDTAVYYCAE | | | | | | |
| | | TGCGGAACATTACCATGAAAGTCGTTTTGGGGGTGGGATCA | | | | | | | HYHESRFGGGISDD | | | | | | |
| | | GCGATGATGCCTTTAATATCTGGGGCCAAGGGACAATGGTC | | | | | | | AFNIWGQGTMVTVS | | | | | | |
| | | ACCGTCTCCTCA | | | | | | | S | | | | | | |
| ADI-81640 | 1286 | CAGGTGCAGCTGTTGGAGTCTGGGGGAGGCTTGGTCCAACC | | | | | | 6836 | QVQLLESGGGLVQP | | 7189 | 24 | | 17 | |
| | | TGGAGGGTCCCTGAGACTCTCCTGTGCAATCTCCGGACTCA | | | | | | | GGSLRLSCAISGLI | | | | | | |
| | | TTATCAGTGACCATTACATAGACTGGGTCCGCCAGGCTCCA | | | | | | | ISDHYIDWVRQAPG | | | | | | |
| | | GGAAAGGGGCTGGAGTGGGTTGGCCGTTCTGCAAACAGAGT | | | | | | | KGLEWVGRSANRVN | | | | | | |
| | | TAACAGTTACACCGCACTCTACGCCGCGTCTGTGATGGGCA | | | | | | | SYTALYAASVMGRF | | | | | | |
| | | GATTCACCATCTCCAGAGATGATTCAAAGAATACCCTGTTT | | | | | | | TISRDDSKNTLFLQ | | | | | | |
| | | CTTCAAATGACCAGTCTGAAAACCGACGACACGGCCGTTTA | | | | | | | MTSLKTDDTAVYHC | | | | | | |
| | | TCACTGTGTTGTCATTGCTGGGACTTATGGCTCCTGGGGCC | | | | | | | VVIAGTYGSWGQGT | | | | | | |
| | | AGGGAACCCTGGTCACCGTCTCCTCA | | | | | | | LVTVSS | | | | | | |
| ADI-81586 | 1286 | CAGGTGCAGCTACAGCAGTGGGGCGCAGGACTGCTGAAGCC | | | | | | 6837 | QVQLQQWGAGLLKP | | 7190 | 21 | | 17 | |
| | | TTCGGAGACCCTGTCCCTCACCTGCGCTTTTTCTGGTGGGT | | | | | | | SETLSLTCAFSGGS | | | | | | |
| | | CCGTCAATGGTGACTACTGGAGCTGGATCCGCCAGCCCCCA | | | | | | | VNGDYWSWIRQPPG | | | | | | |
| | | GGGAAGGGGCTGGAGTTGATTGGGGAGATCAGCCATGATGG | | | | | | | KGLELIGEISHDGT | | | | | | |
| | | AACCACCAAGTACAAGTCGGCCCTCGAGAGTCGAGTCAGTA | | | | | | | TKYKSALESRVSIS | | | | | | |
| | | TATCTTTAGACACGTCCAAGAACCAGTTCTCCCTGAAGTTG | | | | | | | LDTSKNQFSLKLSS | | | | | | |
| | | AGTTCTGTGACCGCCGCGGACACGGCTATGTATTACTGTGC | | | | | | | VTAADTAMYYCARG | | | | | | |
| | | GAGAGGCCTGTCGGCAGTGGTTACTGTTTCTGGGTGGTTCG | | | | | | | LSAVVTVSGWFDSW | | | | | | |
| | | ACTCCTGGGGCCAGGGAACCCTGGTCACCGTCTCCTCA | | | | | | | GQGTLVTVSS | | | | | | |
| ADI-81663 | 1286 | GAGGTGCAGCTGCAGGAGTCTGGGGGAGGCTTGGTCCAGCC | | | | | | 6838 | EVQLQESGGGLVQP | | 7191 | 1 | | 1 | |
| | | TGGAGGGTCCCTGAGACTCTCCTGTGCAGCCTCTGGCTTCA | | | | | | | GGSLRLSCAASGFT | | | | | | |
| | | CCTTCAGTGACCACTTCATGGACTGGGTCCGCCAGGCTCCA | | | | | | | FSDHFMDWVRQAPG | | | | | | |
| | | GGGAAGGGGCTGGAGTGGGTTGGCCGTACTAGAAACAAAGC | | | | | | | KGLEWVGRTRNKAN | | | | | | |
| | | TAACAGTTACACCACAGAATACGCCGCGTCTGTGAAAGGCA | | | | | | | SYTTEYAASVKGRF | | | | | | |
| | | GATTCACCATCTCAAGAGATGATTCAAAGAACTCACTGTAT | | | | | | | TISRDDSKNSLYLQ | | | | | | |
| | | CTGCAAATGAATAGCCTGAAAACCGAGGACACGGCCGTGTA | | | | | | | MNSLKTEDTAVYYC | | | | | | |
| | | TTACTGTGCTAGAGTGGGAGCTGCACTCGGCGGGGTTTGTG | | | | | | | ARVGAALGGVCDYW | | | | | | |
| | | ACTACTGGGGCCAGGGAACCCTGGTCACCGTCTCCTCA | | | | | | | GQGTLVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81829 | 8 | IGHV1- | EVQLV | 5318 | YTFPAY | 5521 | WVRQVP | 5775 | WINPRY | 5874 | RFTMSS | 6180 | ARSGFCD | 6481 | WGQGTL |
| | | 3 | QSGAE | | SIH | | GQRLEW | | GNTKSS | | DTGATT | | GTSCFSG | | VTVSS |
| | | | VRKPG | | | | LG | | QNLQG | | VFMELR | | DY | | |
| | | | ASVKV | | | | | | | | GLTSDD | | | | |
| | | | SCKTV | | | | | | | | TAVYYC | | | | |
| | | | G | | | | | | | | | | | | |
| ADI-81786 | 62 | IGHV4- | EVQLL | 5319 | GSISSH | 5522 | WIRQSP | 434 | NIYHSG | 5875 | RVSITV | 6181 | ARGPHER | 6482 | WGQGTL |
| | | 59 | ESGPG | | YWS | | GKGLEW | | STNYNP | | DTSRTQ | | WFKNWFD | | VTVSS |
| | | | LVKPS | | | | IG | | SLKS | | FSLKLS | | P | | |
| | | | ETLSL | | | | | | | | SVTAAD | | | | |
| | | | ICSIS | | | | | | | | TAVYYC | | | | |
| | | | G | | | | | | | | | | | | |
| ADI-81651 | 24 | IGHV1- | EVQLV | 5320 | YPFTGR | 5523 | WVRQAP | 453 | WINPSS | 5876 | RITLTR | 6182 | TRDPTAG | 6483 | WGQGTL |
| | | 2 | QSGAE | | YMH | | GQGLDW | | GYTNSA | | DTSTTT | | AGDY | | VTVSS |
| | | | VKKPG | | | | MG | | QKFQG | | IFMELN | | | | |
| | | | ASVKV | | | | | | | | SLRSDD | | | | |
| | | | SCKAS | | | | | | | | TAIYYC | | | | |
| | | | G | | | | | | | | | | | | |
| ADI-81725 | 2 | IGHV3- | EVQLV | 5321 | FTFEDY | 208 | WVRQTP | 474 | GISWNG | 633 | RFIISR | 884 | TKAQAPL | 6484 | WGPGTT |
| | | 9 | ESGGG | | AMH | | GKGPEW | | GIMGYA | | DNAKNS | | DSGDFGG | | VTVSS |
| | | | LVQPG | | | | VS | | ASVEG | | LYLQMM | | GFHI | | |
| | | | RSLRL | | | | | | | | RLRAED | | | | |
| | | | SCAAS | | | | | | | | TAFYYC | | | | |
| | | | G | | | | | | | | | | | | |
| ADI-81706 | 76 | IGHV2- | QVPLK | 5322 | FSLATS | 5524 | WIRQPP | 441 | RIDWED | 5877 | RLTISK | 6183 | ARVGDRF | 6485 | WGNGTT |
| | | 70 | ESGPA | | GMRVS | | GKALEW | | DKVYST | | ATSKNQ | | GYQYMDV | | VTVSS |
| | | | LVKPT | | | | LA | | SLKN | | VVLTMT | | | | |
| | | | QTLTL | | | | | | | | DMDPVD | | | | |
| | | | TCTFS | | | | | | | | TATYYC | | | | |
| | | | G | | | | | | | | | | | | |
| ADI-81881 | 58 | IGHV3- | EVQLV | 81 | ITVSSN | 339 | WVRRAP | 5776 | VLYSGG | 5878 | RFTISR | 6184 | ARGDGGG | 6486 | WGQGTT |
| | | 53 | ETGGG | | YMT | | GKGLEW | | STYYAD | | DNSKNT | | DYPYGMD | | VTVSS |
| | | | LIQPG | | | | VS | | SVKG | | LFLQMN | | V | | |
| | | | GSLRL | | | | | | | | SLKAED | | | | |
| | | | SCAAS | | | | | | | | TADYYC | | | | |
| | | | G | | | | | | | | | | | | |
| ADI-81607 | 65 | IGHV3- | QVPLV | 5323 | FTFDDY | 214 | WVRQAP | 437 | GISWNG | 5879 | RFTISR | 6185 | GKGSTSS | 6487 | WGHGTL |
| | | 9 | ESGGG | | AMH | | GKGLEW | | GSIAYA | | DNAKNS | | YYGYFDP | | VTVSS |
| | | | LVQPG | | | | VS | | DSVKG | | LYLQMN | | | | |
| | | | RSLRL | | | | | | | | SLRPED | | | | |
| | | | SCAAS | | | | | | | | TAFYYC | | | | |
| | | | G | | | | | | | | | | | | |
| ADI-81766 | 42 | IGHV3- | EVQLL | 5324 | FTFDDY | 214 | WVRQVP | 436 | GVTWNS | 5880 | RFFISR | 6186 | AKDGGGR | 6488 | WGKGTT |
| | | 9 | ESGGG | | AMH | | GKGLEW | | GTIDYA | | DNAKNS | | LLYFEME | | VTVSS |
| | | | LVQPG | | | | VS | | DSVKG | | LYLQMN | | HYYMDV | | |
| | | | RSLRL | | | | | | | | SLRAED | | | | |
| | | | SCAAS | | | | | | | | TALYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81829 | 1286 | GAGGTCCAGCTGGTGCAGTCTGGGGCTGAGGTGAGGAAGCC | | | | | | 6839 | EVQLVQSGAEVRKP | | 7192 | 33 | | 27 | |
| | | TGGGGCCTCAGTGAAGGTTTCCTGCAAGACTGTTGGATACA | | | | | | | GASVKVSCKTVGYT | | | | | | |
| | | CCTTCCCTGCCTATTCTATCCATTGGGTGCGCCAGGTCCCC | | | | | | | FPAYSIHWVRQVPG | | | | | | |
| | | GGACAACGCCTTGAGTGGCTGGGATGGATCAACCCCCGCTA | | | | | | | QRLEWLGWINPRYG | | | | | | |
| | | CGGAAACACAAAATCTTCACAGAATTTACAGGGCAGATTCA | | | | | | | NTKSSQNLQGRFTM | | | | | | |
| | | CCATGAGCAGTGACACAGGCGCGACCACTGTCTTCATGGAA | | | | | | | SSDTGATTVFMELR | | | | | | |
| | | CTGCGCGGCCTGACTTCTGACGACACGGCTGTCTATTACTG | | | | | | | GLTSDDTAVYYCAR | | | | | | |
| | | TGCGAGATCAGGCTTTTGTGATGGTACGAGTTGCTTCTCGG | | | | | | | SGFCDGTSCFSGDY | | | | | | |
| | | GAGACTACTGGGGCCAGGGAACCCTGGTCACCGTCTCCTCA | | | | | | | WGQGTLVTVSS | | | | | | |
| ADI-81786 | 1286 | GAGGTGCAGCTGTTGGAGTCGGGCCCAGGACTGGTAAAGCC | | | | | | 6840 | EVQLLESGPGLVKP | | 7193 | 11 | | 10 | |
| | | TTCGGAGACCCTGTCCCTCATTTGTTCTATCTCTGGTGGCT | | | | | | | SETLSLICSISGGS | | | | | | |
| | | CCATAAGTAGTCATTACTGGAGCTGGATCCGGCAGTCCCCC | | | | | | | ISSHYWSWIRQSPG | | | | | | |
| | | GGGAAGGGACTGGAATGGATTGGGAATATTTATCACAGTGG | | | | | | | KGLEWIGNIYHSGS | | | | | | |
| | | GAGCACCAATTACAACCCCTCCCTCAAGAGTCGAGTCAGCA | | | | | | | TNYNPSLKSRVSIT | | | | | | |
| | | TTACAGTTGACACGTCCAGGACCCAGTTCTCCCTGAAACTG | | | | | | | VDTSRTQFSLKLSS | | | | | | |
| | | AGTTCTGTGACCGCTGCGGACACGGCCGTCTATTACTGTGC | | | | | | | VTAADTAVYYCARG | | | | | | |
| | | GAGAGGCCCTCATGAGCGGTGGTTCAAGAATTGGTTCGACC | | | | | | | PHERWFKNWFDPWG | | | | | | |
| | | CCTGGGGCCAGGGAACCCTGGTCACCGTCTCCTCA | | | | | | | QGTLVTVSS | | | | | | |
| ADI-81651 | 1286 | GAGGTGCAGCTGGTACAGTCTGGGGCTGAGGTGAAGAAGCC | | | | | | 6841 | EVQLVQSGAEVKKP | | 7194 | 22 | | 15 | |
| | | TGGGGCCTCAGTGAAGGTCTCCTGTAAGGCTTCTGGATACC | | | | | | | GASVKVSCKASGYP | | | | | | |
| | | CCTTCACCGGCCGCTATATGCACTGGGTGCGACAGGCCCCT | | | | | | | FTGRYMHWVRQAPG | | | | | | |
| | | GGACAGGGGCTTGACTGGATGGGATGGATCAACCCTAGCAG | | | | | | | QGLDWMGWINPSSG | | | | | | |
| | | TGGTTACACAAACTCTGCACAGAAGTTTCAGGGCAGGATCA | | | | | | | YTNSAQKFQGRITL | | | | | | |
| | | CCCTGACCAGGGACACGTCCACTACTACAATCTTCATGGAG | | | | | | | TRDTSTTTIFMELN | | | | | | |
| | | CTGAATAGCCTAAGATCTGACGACACAGCCATTTATTACTG | | | | | | | SLRSDDTAIYYCTR | | | | | | |
| | | TACGAGAGATCCGACGGCCGGGGCAGGTGACTACTGGGGCC | | | | | | | DPTAGAGDYWGQGT | | | | | | |
| | | AGGGAACCCTGGTCACCGTCTCCTCA | | | | | | | LVTVSS | | | | | | |
| ADI-81725 | 1295 | TGGCAGGTCCCTGAGACTCTCCTGTGCAGCCTCTGGATTCA | | | | | | 6842 | EVQLVESGGGLVQP | | 7195 | 14 | | 12 | |
| | | CCTTTGAAGATTATGCCATGCACTGGGTCCGGCAAACTCCA | | | | | | | GRSLRLSCAASGFT | | | | | | |
| | | GGGAAGGGCCCGGAGTGGGTCTCAGGTATTAGTTGGAATGG | | | | | | | FEDYAMHWVRQTPG | | | | | | |
| | | GGGTATCATGGGCTATGCGGCCTCTGTAGAGGGCCGATTCA | | | | | | | KGPEWVSGISWNGG | | | | | | |
| | | TCATCTCCAGAGACAACGCCAAGAACTCCCTGTATCTGCAA | | | | | | | IMGYAASVEGRFII | | | | | | |
| | | ATGATGCGTCTGAGAGCAGAGGACACGGCCTTCTATTACTG | | | | | | | SRDNAKNSLYLQMM | | | | | | |
| | | TACAAAAGCCCAAGCCCCACTTGACTCCGGTGACTTCGGCG | | | | | | | RLRAEDTAFYYCTK | | | | | | |
| | | GTGGTTTTCATATCTGGGGCCCAGGGACCACGGTCACCGTC | | | | | | | AQAPLDSGDFGGGF | | | | | | |
| | | TCCTCA | | | | | | | HIWGPGTTVTVSS | | | | | | |
| ADI-81706 | 6803 | CAGGTCCCGCTGAAGGAGTCTGGTCCTGCGCTGGTGAAACC | | | | | | 6843 | QVPLKESGPALVKP | | 7196 | 8 | | 6 | |
| | | CACACAGACCCTCACACTGACCTGCACCTTCTCTGGGTTCT | | | | | | | TQTLTLTCTFSGFS | | | | | | |
| | | CACTCGCTACTAGTGGAATGCGTGTGAGTTGGATCCGTCAG | | | | | | | LATSGMRVSWIRQP | | | | | | |
| | | CCCCCAGGGAAGGCCCTGGAGTGGCTTGCACGCATTGATTG | | | | | | | PGKALEWLARIDWE | | | | | | |
| | | GGAGGATGATAAAGTCTACAGTACTTCTCTAAAGAACAGGC | | | | | | | DDKVYSTSLKNRLT | | | | | | |
| | | TCACCATCTCCAAGGCCACCTCCAAAAACCAGGTGGTCCTT | | | | | | | ISKATSKNQVVLTM | | | | | | |
| | | ACAATGACCGACATGGACCCTGTAGACACAGCCACTTATTA | | | | | | | TDMDPVDTATYYCA | | | | | | |
| | | CTGTGCACGGGTGGGAGACAGATTTGGCTACCAGTACATGG | | | | | | | RVGDRFGYQYMDVW | | | | | | |
| | | ACGTCTGGGGCAATGGGACCACGGTCACCGTCTCCTCA | | | | | | | GNGTTVTVSS | | | | | | |
| ADI-81881 | 1287 | GAGGTGCAGCTGGTGGAGACTGGAGGAGGCTTGATCCAGCC | | | | | | 6844 | EVQLVETGGGLIQP | | 7197 | 8 | | 7 | |
| | | TGGGGGGTCCCTGAGACTCTCCTGTGCAGCCTCTGGGATCA | | | | | | | GGSLRLSCAASGIT | | | | | | |
| | | CCGTCAGCAGCAACTACATGACCTGGGTCCGCCGGGCTCCA | | | | | | | VSSNYMTWVRRAPG | | | | | | |
| | | GGGAAGGGGCTGGAGTGGGTCTCAGTTCTTTATAGCGGTGG | | | | | | | KGLEWVSVLYSGGS | | | | | | |
| | | TAGCACATACTACGCAGACTCCGTGAAGGGCCGATTCACCA | | | | | | | TYYADSVKGRFTIS | | | | | | |
| | | TCTCCAGAGACAATTCCAAGAACACGCTGTTTCTTCAAATG | | | | | | | RDNSKNTLFLQMNS | | | | | | |
| | | AACAGCCTGAAAGCCGAGGACACGGCCGACTATTACTGTGC | | | | | | | LKAEDTADYYCARG | | | | | | |
| | | GAGAGGGGACGGGGGAGGAGACTACCCCTACGGTATGGACG | | | | | | | DGGGDYPYGMDVWG | | | | | | |
| | | TCTGGGGCCAAGGGACCACGGTCACCGTCTCCTCA | | | | | | | QGTTVTVSS | | | | | | |
| ADI-81607 | 6804 | CAGGTGCCGCTGGTGGAGTCTGGGGGAGGCTTGGTACAGCC | | | | | | 6845 | QVPLVESGGGLVQP | | 7198 | 4 | | 4 | |
| | | TGGCAGGTCCCTGAGACTCTCCTGTGCAGCCTCTGGATTCA | | | | | | | GRSLRLSCAASGFT | | | | | | |
| | | CCTTTGATGATTATGCCATGCACTGGGTCCGGCAAGCTCCA | | | | | | | FDDYAMHWVRQAPG | | | | | | |
| | | GGGAAGGGCCTGGAGTGGGTCTCAGGTATTAGTTGGAATGG | | | | | | | KGLEWVSGISWNGG | | | | | | |
| | | TGGTAGCATAGCCTATGCGGACTCTGTGAAGGGCCGATTCA | | | | | | | SIAYADSVKGRFTI | | | | | | |
| | | CCATCTCCAGAGACAACGCCAAGAACTCCCTGTATCTGCAA | | | | | | | SRDNAKNSLYLQMN | | | | | | |
| | | ATGAACAGTCTGAGACCTGAGGACACGGCCTTTTATTACTG | | | | | | | SLRPEDTAFYYCGK | | | | | | |
| | | TGGAAAAGGGTCCACCAGCAGCTATTACGGGTACTTCGACC | | | | | | | GSTSSYYGYFDPWG | | | | | | |
| | | CCTGGGGCCACGGAACCCTGGTCACCGTCTCCTCA | | | | | | | HGTLVTVSS | | | | | | |
| ADI-81766 | 1291 | GAGGTGCAGCTGTTGGAGTCTGGGGGAGGCTTGGTACAGCC | | | | | | 6846 | EVQLLESGGGLVQP | | 7199 | 7 | | 6 | |
| | | TGGCAGGTCCCTGAGACTCTCCTGTGCAGCCTCTGGATTCA | | | | | | | GRSLRLSCAASGFT | | | | | | |
| | | CCTTTGATGATTATGCCATGCACTGGGTCCGACAAGTTCCA | | | | | | | FDDYAMHWVRQVPG | | | | | | |
| | | GGGAAGGGCCTGGAGTGGGTCTCAGGTGTTACTTGGAATAG | | | | | | | KGLEWVSGVTWNSG | | | | | | |
| | | TGGTACCATTGACTATGCGGACTCTGTGAAGGGCCGATTCT | | | | | | | TIDYADSVKGRFFI | | | | | | |
| | | TCATCTCCAGAGACAACGCCAAGAACTCCCTGTATCTGCAA | | | | | | | SRDNAKNSLYLQMN | | | | | | |
| | | ATGAACAGTCTGAGAGCTGAGGACACGGCCTTGTATTACTG | | | | | | | SLRAEDTALYYCAK | | | | | | |
| | | TGCAAAAGATGGAGGTGGACGACTACTGTATTTTGAGATGG | | | | | | | DGGGRLLYFEMEHY | | | | | | |
| | | AGCACTACTACATGGACGTCTGGGGCAAAGGGACCACGGTC | | | | | | | YMDVWGKGTTVTVS | | | | | | |
| | | ACCGTCTCCTCA | | | | | | | S | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81687 | 57 | IGHV3- | EVQLV | 5325 | FTFNYA | 5525 | WVRQAP | 5777 | RVKSHI | 5881 | RFVISR | 6187 | VTDLSRF | 6489 | WGQGTT |
| | | 15 | ESGGG | | WMT | | GGGLEW | | DGGTTD | | DDSKNT | | LSSRETD | | VTVSS |
| | | | FVKPG | | | | VG | | YAAPVK | | LYLEMN | | AFDI | | |
| | | | GSLRL | | | | | | G | | SLKTED | | | | |
| | | | SCAAS | | | | | | | | TAVYYC | | | | |
| | | | G | | | | | | | | | | | | |
| ADI-81908 | 82 | IGHV3- | EVQLV | 5326 | FTFRTY | 5526 | WVRQTP | 5778 | VVSSDA | 5882 | RFTISR | 6188 | AREKRVR | 6490 | WGQGTT |
| | | 30 | ESGGR | | DFY | | GKGLEW | | SSKFYA | | DNSKNT | | TILGHYG | | VTVSS |
| | | | AVQPG | | | | VA | | DSVKG | | LFLQMS | | FDV | | |
| | | | GSLRL | | | | | | | | SLIPED | | | | |
| | | | SCGAS | | | | | | | | SAVYYC | | | | |
| | | | G | | | | | | | | | | | | |
| ADI-81827 | 7 | IGHV3- | EVQLV | 5327 | FTFGNF | 5527 | WVRQTP | 5778 | ATSHDG | 5883 | RFTISR | 6189 | ARGGHIV | 6491 | WGQGTP |
| | | 30 | QSGGG | | AVH | | GKGLEW | | SEKNYA | | DNSKNT | | MVTAGYF | | VTVSS |
| | | | VVQPG | | | | VA | | DSVKG | | LYLQMD | | DF | | |
| | | | RSLRL | | | | | | | | SLRPED | | | | |
| | | | SCVAS | | | | | | | | TALYYC | | | | |
| | | | G | | | | | | | | | | | | |
| ADI-81875 | 52 | IGHV3- | EVQLV | 5328 | FTFRHF | 5528 | WVRQTP | 5778 | VISYDG | 5884 | RFTISR | 6190 | AKDHLTA | 6492 | WGQGTL |
| | | 30 | ESGGG | | ALH | | GKGLEW | | NEKYYA | | DNSKNT | | RYYDFRS | | VTVSS |
| | | | VVQPG | | | | VA | | DSVKG | | VYLQMN | | VSFNGDA | | |
| | | | RSLSL | | | | | | | | SLRVED | | Y | | |
| | | | SCAAS | | | | | | | | TAVYFC | | | | |
| | | | G | | | | | | | | | | | | |
| ADI-81885 | 61 | IGHV4- | EVQLV | 5329 | YSISSG | 338 | WVRLPP | 5779 | SVSHDG | 5885 | RVTISA | 6191 | AREGGSF | 6493 | WGQGTL |
| | | 38-2 | ESGPG | | YSWG | | GKGLQW | | STYHNP | | DTSKNQ | | FRDGDFE | | VTVSS |
| | | | VVRPS | | | | IG | | SLKS | | FSLMLS | | H | | |
| | | | ETLSL | | | | | | | | SVTAAD | | | | |
| | | | TCAVS | | | | | | | | TAIYYC | | | | |
| | | | G | | | | | | | | | | | | |
| ADI-81638 | 11 | IGHV3- | EVQLK | 5330 | FTLSSF | 5529 | WVRQAP | 437 | YISSGS | 5886 | RFTISR | 6192 | ARHLSPT | 6494 | WGQGTT |
| | | 48 | ESGGG | | SMN | | GKGLEW | | TIIHYA | | DNAKNS | | NNDFLTG | | VTVSS |
| | | | VVQPG | | | | VS | | DSMRG | | LFLQLN | | PDAFDL | | |
| | | | GSLRL | | | | | | | | SLRVED | | | | |
| | | | SCGAS | | | | | | | | TAVYYC | | | | |
| | | | G | | | | | | | | | | | | |
| ADI-81554 | 20 | IGHV3- | QVQLQ | 5331 | FSFNHA | 5530 | WVRQAP | 432 | RIKTIN | 5887 | RFTISR | 6193 | STASDYG | 6495 | WGQGTL |
| | | 15 | QSGGG | | WMS | | GKGLEW | | DGGTTD | | DDSKST | | DHSPHY | | VTVSS |
| | | | FVKPG | | | | VG | | YAAPVK | | LSLQMN | | | | |
| | | | GSLRL | | | | | | G | | SLKTED | | | | |
| | | | SCATS | | | | | | | | TAVYFC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81687 | 1287 | GAGGTGCAGCTGGTGGAGTCTGGGGGAGGCTTTGTAAAGCC | | | | | | 6847 | EVQLVESGGGFVKP | | 7200 | 14 | | 10 | |
| | | TGGGGGGTCCCTTAGACTCTCCTGTGCAGCCTCTGGATTCA | | | | | | | GGSLRLSCAASGFT | | | | | | |
| | | CTTTCAATTACGCCTGGATGACCTGGGTCCGCCAGGCTCCA | | | | | | | FNYAWMTWVRQAPG | | | | | | |
| | | GGGGGGGGGCTGGAGTGGGTTGGCCGTGTTAAAAGCCACAT | | | | | | | GGLEWVGRVKSHID | | | | | | |
| | | TGATGGTGGGACAACAGACTACGCGGCACCCGTGAAAGGCA | | | | | | | GGTTDYAAPVKGRF | | | | | | |
| | | GATTCGTTATTTCCAGAGATGATTCAAAAAACACGCTGTAT | | | | | | | VISRDDSKNTLYLE | | | | | | |
| | | TTGGAAATGAACAGCCTGAAAACCGAGGACACCGCCGTATA | | | | | | | MNSLKTEDTAVYYC | | | | | | |
| | | TTACTGTGTCACAGATCTCTCAAGGTTTTTATCGTCCAGGG | | | | | | | VTDLSRFLSSRETD | | | | | | |
| | | AGACTGATGCTTTTGACATCTGGGGCCAAGGGACCACGGTC | | | | | | | AFDIWGQGTTVTVS | | | | | | |
| | | ACCGTCTCCTCA | | | | | | | S | | | | | | |
| ADI-81908 | 1287 | TGGGGGGTCCCTGAGACTCTCCTGTGGAGCCTCTGGATTCA | | | | | | 6848 | EVQLVESGGRAVQP | | 7201 | 22 | | 19 | |
| | | CCTTTAGGACCTATGATTTTTACTGGGTCCGCCAGACTCCA | | | | | | | GGSLRLSCGASGFT | | | | | | |
| | | GGCAAGGGGCTGGAGTGGGTGGCAGTTGTCTCATCTGATGC | | | | | | | FRTYDFYWVRQTPG | | | | | | |
| | | AAGCAGTAAATTCTACGCAGACTCCGTGAAGGGCCGATTCA | | | | | | | KGLEWVAVVSSDAS | | | | | | |
| | | CCATCTCCAGAGACAATTCCAAGAACACATTGTTTCTGCAA | | | | | | | SKFYADSVKGRFTI | | | | | | |
| | | ATGAGCAGCCTGATACCTGAGGACAGTGCTGTTTACTACTG | | | | | | | SRDNSKNTLFLQMS | | | | | | |
| | | TGCGAGAGAGAAGCGCGTGAGAACGATTCTTGGGCACTACG | | | | | | | SLIPEDSAVYYCAR | | | | | | |
| | | GTTTCGACGTCTGGGGCCAAGGGACCACGGTCACCGTCTCC | | | | | | | EKRVRTILGHYGFD | | | | | | |
| | | TCA | | | | | | | VWGQGTTVTVSS | | | | | | |
| ADI-81827 | 6805 | GAGGTGCAGCTGGTGCAGTCTGGGGGAGGCGTGGTCCAGCC | | | | | | 6849 | EVQLVQSGGGVVQP | | 7202 | 20 | | 14 | |
| | | TGGGAGGTCCCTGAGACTCTCCTGTGTGGCCTCTGGCTTCA | | | | | | | GRSLRLSCVASGFT | | | | | | |
| | | CCTTCGGAAATTTTGCTGTGCACTGGGTCCGCCAGACTCCG | | | | | | | FGNFAVHWVRQTPG | | | | | | |
| | | GGCAAGGGGCTAGAGTGGGTGGCAGCTACTTCACATGATGG | | | | | | | KGLEWVAATSHDGS | | | | | | |
| | | AAGTGAGAAAAACTACGCAGACTCCGTGAAGGGCCGCTTCA | | | | | | | EKNYADSVKGRFTI | | | | | | |
| | | CCATCTCCAGAGACAATTCCAAGAACACGCTGTATCTGCAA | | | | | | | SRDNSKNTLYLQMD | | | | | | |
| | | ATGGACAGCCTGAGACCTGAGGACACGGCTCTATACTATTG | | | | | | | SLRPEDTALYYCAR | | | | | | |
| | | TGCGAGAGGGGGACATATTGTAATGGTGACTGCTGGTTACT | | | | | | | GGHIVMVTAGYFDF | | | | | | |
| | | TTGACTTCTGGGGCCAGGGAACCCCGGTCACCGTCTCCTCA | | | | | | | WGQGTPVTVSS | | | | | | |
| ADI-81875 | 1286 | GAGGTGCAGCTGGTGGAGTCTGGGGGAGGCGTGGTCCAGCC | | | | | | 6850 | EVQLVESGGGVVQP | | 7203 | 17 | | 12 | |
| | | TGGGAGGTCCCTGAGCCTCTCCTGTGCGGCCTCTGGATTCA | | | | | | | GRSLSLSCAASGFT | | | | | | |
| | | CCTTCCGTCATTTTGCCCTACACTGGGTCCGCCAGACTCCA | | | | | | | FRHFALHWVRQTPG | | | | | | |
| | | GGCAAGGGCCTGGAATGGGTGGCAGTTATTTCGTATGATGG | | | | | | | KGLEWVAVISYDGN | | | | | | |
| | | AAATGAAAAATACTACGCAGACTCCGTGAAGGGCCGATTCA | | | | | | | EKYYADSVKGRFTI | | | | | | |
| | | CCATCTCCAGAGACAATTCCAAGAACACAGTATATCTGCAA | | | | | | | SRDNSKNTVYLQMN | | | | | | |
| | | ATGAACAGCCTGAGAGTTGAGGACACGGCTGTTTATTTCTG | | | | | | | SLRVEDTAVYFCAK | | | | | | |
| | | TGCGAAAGATCATTTAACCGCCAGGTACTACGATTTTCGGA | | | | | | | DHLTARYYDFRSVS | | | | | | |
| | | GTGTTTCTTTTAATGGTGACGCCTACTGGGGCCAGGGCACC | | | | | | | FNGDAYWGQGTLVT | | | | | | |
| | | CTGGTCACCGTCTCCTCA | | | | | | | VSS | | | | | | |
| ADI-81885 | 1286 | GAGGTGCAGCTGGTGGAGTCGGGCCCAGGAGTGGTGAGGCC | | | | | | 6851 | EVQLVESGPGVVRP | | 7204 | 17 | | 13 | |
| | | TTCGGAGACCCTGTCCCTCACCTGCGCTGTGTCTGGTTACT | | | | | | | SETLSLTCAVSGYS | | | | | | |
| | | CGATCAGTAGTGGTTACTCATGGGGCTGGGTGCGGCTGCCC | | | | | | | ISSGYSWGWVRLPP | | | | | | |
| | | CCAGGGAAGGGTCTGCAGTGGATTGGGAGTGTCTCTCATGA | | | | | | | GKGLQWIGSVSHDG | | | | | | |
| | | TGGGAGCACCTACCACAACCCGTCCCTCAAGAGTCGAGTGA | | | | | | | STYHNPSLKSRVTI | | | | | | |
| | | CCATATCAGCAGACACGTCCAAGAACCAGTTCTCCCTGATG | | | | | | | SADTSKNQFSLMLS | | | | | | |
| | | CTGAGCTCTGTGACCGCCGCAGACACGGCCATCTATTACTG | | | | | | | SVTAADTAIYYCAR | | | | | | |
| | | TGCGAGAGAGGGTGGGAGTTTTTTCCGGGATGGTGACTTCG | | | | | | | EGGSFFRDGDFEHW | | | | | | |
| | | AACACTGGGGCCAGGGCACCCTGGTCACCGTCTCCTCA | | | | | | | GQGTLVTVSS | | | | | | |
| ADI-81638 | 1287 | GAGGTGCAGCTGAAGGAGTCTGGGGGAGGCGTGGTACAGCC | | | | | | 6852 | EVQLKESGGGVVQP | | 7205 | 14 | | 13 | |
| | | GGGGGGGTCCCTGAGACTCTCCTGTGGAGCCTCTGGATTCA | | | | | | | GGSLRLSCGASGFT | | | | | | |
| | | CGTTAAGTAGTTTCAGCATGAATTGGGTCCGCCAGGCTCCA | | | | | | | LSSFSMNWVRQAPG | | | | | | |
| | | GGGAAGGGGCTGGAGTGGGTTTCATATATTAGTAGTGGTAG | | | | | | | KGLEWVSYISSGST | | | | | | |
| | | TACTATCATACACTATGCAGACTCTATGAGGGGCCGATTCA | | | | | | | IIHYADSMRGRFTI | | | | | | |
| | | CCATCTCCAGGGACAATGCCAAGAACTCACTGTTTCTACAA | | | | | | | SRDNAKNSLFLQLN | | | | | | |
| | | CTGAACAGCCTGAGAGTCGAAGACACGGCTGTGTATTACTG | | | | | | | SLRVEDTAVYYCAR | | | | | | |
| | | TGCGAGACATCTCTCTCCAACGAATAACGATTTTTTGACTG | | | | | | | HLSPTNNDFLTGPD | | | | | | |
| | | GCCCAGATGCTTTTGATCTCTGGGGTCAAGGGACCACGGTC | | | | | | | AFDLWGQGTTVTVS | | | | | | |
| | | ACCGTCTCCTCA | | | | | | | S | | | | | | |
| ADI-81554 | 1286 | CAGGTGCAGCTGCAGCAGTCTGGGGGAGGCTTCGTAAAGCC | | | | | | 6853 | QVQLQQSGGGFVKP | | 7206 | 13 | | 11 | |
| | | TGGTGGGTCCCTTAGACTCTCCTGTGCAACCTCTGGATTCT | | | | | | | GGSLRLSCATSGFS | | | | | | |
| | | CTTTTAATCACGCCTGGATGAGCTGGGTCCGCCAGGCTCCA | | | | | | | FNHAWMSWVRQAPG | | | | | | |
| | | GGGAAGGGGCTGGAGTGGGTTGGCCGTATTAAAACCATAAA | | | | | | | KGLEWVGRIKTIND | | | | | | |
| | | TGATGGTGGGACAACAGACTACGCTGCACCCGTGAAAGGCA | | | | | | | GGTTDYAAPVKGRF | | | | | | |
| | | GATTCACCATTTCAAGAGATGATTCGAAAAGCACACTGAGT | | | | | | | TISRDDSKSTLSLQ | | | | | | |
| | | CTCCAAATGAACAGCCTGAAAACCGAGGACACAGCCGTCTA | | | | | | | MNSLKTEDTAVYFC | | | | | | |
| | | TTTTTGTAGCACAGCTAGTGACTACGGTGACCACTCCCCTC | | | | | | | STASDYGDHSPHYW | | | | | | |
| | | ACTACTGGGGCCAGGGAACCCTGGTCACCGTCTCCTCA | | | | | | | GQGTLVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81756 | 32 | IGHV3- | EVQLV | 5332 | FRFSNY | 5531 | WVRQAI | 5780 | VIGTDG | 5888 | RFTVSR | 6194 | VRGAWSS | 6496 | WGQGAL |
| | | 13 | ESGGG | | DMH | | GKGLEW | | DTYYPD | | DNGKNS | | DSYLPRG | | VTVSS |
| | | | LVQPG | | | | LS | | SVKG | | LYLQMN | | FFDY | | |
| | | | GSLRL | | | | | | | | NLGAGD | | | | |
| | | | SCAAS | | | | | | | | TAVYYC | | | | |
| | | | G | | | | | | | | | | | | |
| ADI-81773 | 49 | IGHV3- | EVQLV | 5333 | FTFSSD | 5532 | WVRQPA | 5781 | AIGTSG | 5889 | RFTISR | 6195 | ARGTQQQ | 6497 | WGKGTT |
| | | 13 | ESGGG | | DMH | | GKGLEW | | DSYYTD | | ENAKNS | | LVLHGNV | | VTVSS |
| | | | LVQPG | | | | VS | | SVKG | | LFLQMN | | NYFYYMD | | |
| | | | GSLRL | | | | | | | | NLRAGD | | V | | |
| | | | SCVAS | | | | | | | | TAVYYC | | | | |
| | | | G | | | | | | | | | | | | |
| ADI-81575 | 37 | IGHV3- | QVQLK | 5334 | LIVSSN | 343 | WVRQAP | 5782 | VIYAGG | 5890 | RFTISR | 6196 | ARGDGGY | 6498 | WGQGTL |
| | | 53 | QSGGG | | YMS | | GKGLEL | | RTDYAD | | DDSKNT | | YSGIDY | | VTVSS |
| | | | LIQPG | | | | VS | | SVKG | | LHLQMN | | | | |
| | | | GSLRL | | | | | | | | SLRVED | | | | |
| | | | SCAAS | | | | | | | | TAVYYC | | | | |
| | | | G | | | | | | | | | | | | |
| ADI-81727 | 4 | IGHV4- | EVQLL | 5335 | ASISTY | 5533 | WIRQPP | 445 | YIYYSG | 5891 | RVTISV | 6197 | ARLPDSD | 6499 | WGQGTL |
| | | 59 | ESGPG | | YWG | | GKGLEW | | SANLNP | | DTSKNQ | | YHGYAYY | | VTVSS |
| | | | LVKPS | | | | IG | | SLKS | | LSLKLN | | FDY | | |
| | | | ETLSL | | | | | | | | SVTAAD | | | | |
| | | | TCTVS | | | | | | | | TAIYYC | | | | |
| | | | G | | | | | | | | | | | | |
| ADI-81704 | 74 | IGHV3- | EVQLV | 5336 | FTFSNY | 351 | WVRQGP | 493 | YISTSG | 686 | RVTISR | 6198 | LTHPQPQ | 6500 | WGQGTL |
| | | 48 | ESGGD | | EMN | | GKGLEW | | STYYYA | | DNARNS | | YVRPTPV | | VTVSQ |
| | | | LVQPG | | | | IS | | DSVKG | | LYLEMH | | VWFDP | | |
| | | | GSLRL | | | | | | | | SLRVED | | | | |
| | | | SCVAS | | | | | | | | TGLYYC | | | | |
| | | | G | | | | | | | | | | | | |
| ADI-81768 | 44 | IGHV1- | QVQLV | 5337 | GTFSNF | 5534 | WVRQAP | 477 | RVIPVL | 5892 | RVTIIA | 6199 | ARDVKFD | 6501 | WGQGTT |
| | | 69 | QSGAE | | AIN | | GQGPEW | | DRPIYA | | DTSTNT | | YSTSSIE | | VTVSS |
| | | | VKKPG | | | | MG | | QQFQG | | AYMEVS | | ESIAFDI | | |
| | | | SSVKV | | | | | | | | SLRSED | | | | |
| | | | SCRAS | | | | | | | | TAVYYC | | | | |
| | | | G | | | | | | | | | | | | |
| ADI-81775 | 51 | IGHV3- | EVQLV | 5332 | ITFNND | 5535 | WVRQGP | 5783 | AIGSSG | 5893 | RFTISR | 6200 | ARGDIEV | 6502 | WGKGTT |
| | | 13 | ESGGG | | DIH | | GKGLEW | | DTYYAD | | ENARKS | | RPASTTH | | VTVSS |
| | | | LVQPG | | | | VS | | SVKG | | LYLQMN | | KTSYFYY | | |
| | | | GSLRL | | | | | | | | NLRAGD | | MDV | | |
| | | | SCAAS | | | | | | | | TGIYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81756 | 1285 | GGGGGGGTCCCTGAGACTCTCCTGTGCAGCCTCTGGATTCA | | | | | | 6854 | EVQLVESGGGLVQP | | 7207 | 13 | | 12 | |
| | | GGTTCAGTAACTACGACATGCACTGGGTCCGCCAGGCTATA | | | | | | | GGSLRLSCAASGFR | | | | | | |
| | | GGAAAAGGTCTAGAGTGGCTCTCAGTTATTGGTACTGATGG | | | | | | | FSNYDMHWVRQAIG | | | | | | |
| | | GGACACATACTATCCAGACTCCGTGAAGGGCCGATTCACCG | | | | | | | KGLEWLSVIGTDGD | | | | | | |
| | | TCTCCAGAGACAATGGCAAGAACTCCTTGTATCTTCAAATG | | | | | | | TYYPDSVKGRFTVS | | | | | | |
| | | AACAACTTGGGAGCCGGGGACACGGCTGTGTATTATTGTGT | | | | | | | RDNGKNSLYLQMNN | | | | | | |
| | | AAGAGGGGCTTGGAGCAGTGACTCATATCTCCCCCGTGGAT | | | | | | | LGAGDTAVYYCVRG | | | | | | |
| | | TTTTTGACTACTGGGGCCAGGGAGCCCTGGTCACCGTCTCC | | | | | | | AWSSDSYLPRGFFD | | | | | | |
| | | TCA | | | | | | | YWGQGALVTVSS | | | | | | |
| ADI-81773 | 1291 | GAGGTGCAGCTGGTGGAGTCTGGGGGAGGCTTGGTGCAGCC | | | | | | 6855 | EVQLVESGGGLVQP | | 7208 | 11 | | 10 | |
| | | TGGGGGGTCCCTGAGACTCTCCTGTGTAGCCTCTGGATTCA | | | | | | | GGSLRLSCVASGFT | | | | | | |
| | | CCTTCAGTAGTGACGACATGCACTGGGTCCGCCAACCTGCA | | | | | | | FSSDDMHWVRQPAG | | | | | | |
| | | GGTAAAGGTCTGGAGTGGGTCTCAGCTATTGGTACTTCTGG | | | | | | | KGLEWVSAIGTSGD | | | | | | |
| | | TGACTCCTACTATACAGACTCCGTGAAGGGCCGGTTCACCA | | | | | | | SYYTDSVKGRFTIS | | | | | | |
| | | TCTCCAGAGAAAATGCCAAGAACTCCTTGTTTCTTCAAATG | | | | | | | RENAKNSLFLQMNN | | | | | | |
| | | AACAACCTGAGAGCCGGGGACACGGCTGTGTATTATTGCGC | | | | | | | LRAGDTAVYYCARG | | | | | | |
| | | AAGAGGTACACAGCAGCAACTGGTACTTCATGGAAACGTTA | | | | | | | TQQQLVLHGNVNYF | | | | | | |
| | | ACTACTTTTATTACATGGACGTCTGGGGCAAAGGGACCACG | | | | | | | YYMDVWGKGTTVTV | | | | | | |
| | | GTCACCGTCTCCTCA | | | | | | | SS | | | | | | |
| ADI-81575 | 1286 | CAGGTGCAGCTGAAGCAGTCTGGAGGAGGCTTGATCCAACC | | | | | | 6856 | QVQLKQSGGGLIQP | | 7209 | 10 | | 9 | |
| | | TGGGGGGTCCCTGAGACTCTCCTGTGCAGCCTCTGGACTCA | | | | | | | GGSLRLSCAASGLI | | | | | | |
| | | TCGTCAGTAGCAACTACATGAGCTGGGTCCGCCAGGCTCCA | | | | | | | VSSNYMSWVRQAPG | | | | | | |
| | | GGGAAGGGGCTGGAGTTGGTCTCAGTTATCTATGCCGGTGG | | | | | | | KGLELVSVIYAGGR | | | | | | |
| | | TAGAACAGACTACGCAGACTCCGTGAAGGGCCGATTCACCA | | | | | | | TDYADSVKGRFTIS | | | | | | |
| | | TCTCCAGAGACGATTCCAAGAACACGCTACATCTTCAAATG | | | | | | | RDDSKNTLHLQMNS | | | | | | |
| | | AACAGCCTGAGAGTCGAAGACACGGCCGTGTATTATTGTGC | | | | | | | LRVEDTAVYYCARG | | | | | | |
| | | GAGAGGTGATGGGGGATATTATAGTGGCATCGACTACTGGG | | | | | | | DGGYYSGIDYWGQG | | | | | | |
| | | GCCAGGGAACCCTGGTCACCGTCTCCTCA | | | | | | | TLVTVSS | | | | | | |
| ADI-81727 | 1286 | GAGGTGCAGCTGTTGGAGTCGGGCCCAGGACTGGTGAAGCC | | | | | | 6857 | EVQLLESGPGLVKP | | 7210 | 10 | | 8 | |
| | | CTCGGAGACCCTGTCCCTCACGTGCACGGTCTCTGGTGCCT | | | | | | | SETLSLTCTVSGAS | | | | | | |
| | | CCATCAGTACTTACTACTGGGGCTGGATCCGGCAGCCCCCA | | | | | | | ISTYYWGWIRQPPG | | | | | | |
| | | GGGAAGGGACTGGAGTGGATTGGGTATATCTACTACAGTGG | | | | | | | KGLEWIGYIYYSGS | | | | | | |
| | | GAGCGCCAATCTCAACCCCTCCCTCAAGAGTCGAGTCACCA | | | | | | | ANLNPSLKSRVTIS | | | | | | |
| | | TATCGGTGGACACGTCCAAAAACCAGTTGTCCCTGAAGCTG | | | | | | | VDTSKNQLSLKLNS | | | | | | |
| | | AACTCTGTGACCGCTGCGGACACGGCCATTTATTACTGTGC | | | | | | | VTAADTAIYYCARL | | | | | | |
| | | GAGGCTCCCTGATAGTGACTATCACGGATACGCGTACTACT | | | | | | | PDSDYHGYAYYFDY | | | | | | |
| | | TTGACTACTGGGGCCAGGGAACCCTGGTCACCGTCTCCTCA | | | | | | | WGQGTLVTVSS | | | | | | |
| ADI-81704 | 6806 | TGGAGGGTCCCTGAGACTCTCCTGTGTCGCCTCTGGATTCA | | | | | | 6858 | EVQLVESGGDLVQP | | 7211 | 18 | | 14 | |
| | | CCTTCAGTAATTATGAGATGAACTGGGTCCGCCAGGGTCCA | | | | | | | GGSLRLSCVASGFT | | | | | | |
| | | GGGAAGGGGCTGGAGTGGATTTCATACATTAGTACGAGTGG | | | | | | | FSNYEMNWVRQGPG | | | | | | |
| | | AAGTACCTATTACTACGCAGACTCTGTGAAGGGCCGAGTCA | | | | | | | KGLEWISYISTSGS | | | | | | |
| | | CCATATCCAGAGACAACGCCAGGAATTCACTGTATCTGGAA | | | | | | | TYYYADSVKGRVTI | | | | | | |
| | | ATGCACAGCCTGAGAGTCGAAGACACTGGTCTTTATTACTG | | | | | | | SRDNARNSLYLEMH | | | | | | |
| | | TCTGACACACCCTCAGCCTCAATATGTACGACCCACGCCGG | | | | | | | SLRVEDTGLYYCLT | | | | | | |
| | | TGGTTTGGTTCGACCCCTGGGGCCAGGGAACCTTGGTCACC | | | | | | | HPQPQYVRPTPVVW | | | | | | |
| | | GTCTCTCAG | | | | | | | FDPWGQGTLVTVSQ | | | | | | |
| ADI-81768 | 1287 | CAGGTGCAGCTGGTGCAGTCTGGGGCTGAAGTGAAGAAGCC | | | | | | 6859 | QVQLVQSGAEVKKP | | 7212 | 17 | | 16 | |
| | | TGGGTCCTCGGTGAAGGTCTCCTGCAGGGCTTCTGGAGGCA | | | | | | | GSSVKVSCRASGGT | | | | | | |
| | | CCTTCAGCAACTTTGCTATCAACTGGGTGCGACAGGCCCCT | | | | | | | FSNFAINWVRQAPG | | | | | | |
| | | GGACAAGGGCCTGAGTGGATGGGAAGGGTCATCCCTGTCCT | | | | | | | QGPEWMGRVIPVLD | | | | | | |
| | | TGATAGACCAATCTACGCACAACAATTCCAGGGCAGAGTCA | | | | | | | RPIYAQQFQGRVTI | | | | | | |
| | | CGATTATCGCGGACACATCCACGAACACAGCCTACATGGAG | | | | | | | IADTSTNTAYMEVS | | | | | | |
| | | GTGAGCAGCCTGAGATCCGAAGACACGGCCGTTTATTATTG | | | | | | | SLRSEDTAVYYCAR | | | | | | |
| | | TGCGAGAGATGTGAAGTTCGACTATAGCACCTCGTCCATTG | | | | | | | DVKFDYSTSSIEES | | | | | | |
| | | AGGAGAGCATCGCTTTTGATATCTGGGGCCAGGGGACCACG | | | | | | | IAFDIWGQGTTVTV | | | | | | |
| | | GTCACCGTCTCCTCA | | | | | | | SS | | | | | | |
| ADI-81775 | 1291 | GAGGTGCAGCTGGTGGAGTCTGGGGGAGGCTTGGTACAGCC | | | | | | 6860 | EVQLVESGGGLVQP | | 7213 | 17 | | 16 | |
| | | GGGGGGGTCCCTGAGACTCTCTTGTGCAGCCTCTGGAATCA | | | | | | | GGSLRLSCAASGIT | | | | | | |
| | | CCTTCAATAACGACGACATACACTGGGTCCGCCAAGGTCCA | | | | | | | FNNDDIHWVRQGPG | | | | | | |
| | | GGGAAAGGTCTGGAGTGGGTCTCAGCTATTGGTTCTTCTGG | | | | | | | KGLEWVSAIGSSGD | | | | | | |
| | | TGACACATATTATGCAGACTCCGTGAAGGGCCGATTCACCA | | | | | | | TYYADSVKGRFTIS | | | | | | |
| | | TCTCCAGAGAAAACGCCAGGAAGTCCTTGTATCTCCAAATG | | | | | | | RENARKSLYLQMNN | | | | | | |
| | | AACAACCTGAGAGCCGGGGACACAGGTATATATTACTGTGC | | | | | | | LRAGDTGIYYCARG | | | | | | |
| | | GAGAGGGGATATTGAAGTAAGACCGGCCAGTACCACGCACA | | | | | | | DIEVRPASTTHKTS | | | | | | |
| | | AGACCTCTTACTTTTACTACATGGACGTCTGGGGCAAAGGG | | | | | | | YFYYMDVWGKGTTV | | | | | | |
| | | ACCACGGTCACCGTCTCCTCA | | | | | | | TVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81598 | 58 | IGHV3- | QVQLV | 12 | FTFSRF | 5536 | WFRQGT | 5784 | AIGTSG | 5894 | RFTISR | 6201 | ARATPHS | 6503 | WGQGTL |
| | | 13 | QSGGG | | DMH | | GKGLEW | | DTYYGD | | ENAKNS | | SGYWDWF | | VTVSS |
| | | | LVQPG | | | | VS | | SVKG | | LNLQMN | | DP | | |
| | | | GSLRL | | | | | | | | SLRAED | | | | |
| | | | SCAAS | | | | | | | | TAIYYC | | | | |
| | | | G | | | | | | | | | | | | |
| ADI-81914 | 88 | IGHV3- | EVQLV | 5338 | FTFGNY | 5537 | WVRQAP | 5785 | VVSYDG | 5895 | RFSISR | 6202 | VRPHLPD | 6504 | WGQGVL |
| | | 30 | ESGGG | | AMH | | DKGLEW | | SNEFYA | | DNSKNT | | YFGSGSF | | VTVSS |
| | | | VVQPG | | | | VA | | DSVKG | | LHLQMN | | HY | | |
| | | | RSLRL | | | | | | | | SLKAED | | | | |
| | | | SCAAS | | | | | | | | TAEYFC | | | | |
| | | | G | | | | | | | | | | | | |
| ADI-81637 | 10 | IGHV1- | EVQLV | 5339 | GAFSIH | 5538 | WVRQAP | 5786 | GIIPMF | 5896 | RVTITA | 6203 | ARDGRYC | 6505 | WGQGTL |
| | | 69 | QSGPA | | GIT | | GQGLEW | | GTANYP | | EESTST | | NSTACLN | | VTVSS |
| | | | VKKPG | | | | LG | | QNFQG | | VYMELN | | FFDL | | |
| | | | SSVKV | | | | | | | | SLRCED | | | | |
| | | | SCKAS | | | | | | | | TAMYYC | | | | |
| | | | G | | | | | | | | | | | | |
| ADI-81641 | 14 | IGHV4- | EVQLL | 5340 | DSISSG | 5539 | WVRQPP | 5787 | DIYHSG | 5897 | RVTISV | 6204 | ARSIGSR | 6506 | WGQGTL |
| | | 4 | ESAPG | | YWWS | | LKGLEW | | NTVYNP | | DKSKNH | | PRWFDP | | VTVSS |
| | | | LVKPS | | | | IG | | SLKS | | FSLSLK | | | | |
| | | | GTLSL | | | | | | | | SVTAAD | | | | |
| | | | TCAVS | | | | | | | | TAVYYC | | | | |
| | | | G | | | | | | | | | | | | |
| ADI-81729 | 6 | IGHV1- | EVQLL | 5341 | YTFTNN | 5540 | WVRQAP | 433 | IINPSG | 5898 | RVTLTS | 6205 | ARDSEYI | 6507 | WGQGTL |
| | | 46 | ESGTE | | YIH | | GQGLEW | | GATTYA | | DTSTST | | PAAGSFD | | VTVSS |
| | | | VKKPG | | | | MG | | QKFQG | | VYLELT | | Y | | |
| | | | ASVKV | | | | | | | | SLTSED | | | | |
| | | | SCKAL | | | | | | | | TAVYYC | | | | |
| | | | G | | | | | | | | | | | | |
| ADI-81643 | 16 | IGHV5- | EVQLV | 5342 | YSFISY | 5541 | WVRQMP | 476 | NIDPSD | 5899 | HVTIAA | 6206 | ARRIMIT | 6508 | WGQGTT |
| | | 10-1 | ESGPE | | WIT | | GKGLEW | | SYTNYS | | DKGIST | | SGGKEID | | VTVSS |
| | | | VKKPG | | | | MG | | PAFQG | | AYLQWR | | AFDI | | |
| | | | ESLTI | | | | | | | | SLKASD | | | | |
| | | | SCKAS | | | | | | | | TAMYYC | | | | |
| | | | G | | | | | | | | | | | | |
| ADI-81701 | 71 | IGHV1- | EVQLV | 5343 | YAFINY | 5542 | WVRQAP | 433 | WISPYD | 5900 | RVTMAT | 6207 | VRDYYGA | 6509 | WGQGTL |
| | | 18 | QSGAE | | GIS | | GQGLEW | | GHTNYA | | DTSAST | | LTSPNAN | | VTVSS |
| | | | VKKPG | | | | MG | | QKIQG | | AYMDLR | | SETLGTM | | |
| | | | ASVKV | | | | | | | | SLRSDD | | DY | | |
| | | | SCKTS | | | | | | | | TAVYYC | | | | |
| | | | G | | | | | | | | | | | | |
| ADI-81720 | 90 | IGHV3- | EVQLV | 5344 | FTFSRH | 5543 | WVRQAP | 431 | LIYYDG | 5901 | RFTISR | 6208 | ARDGANG | 6510 | WGQGTL |
| | | 33 | ESGGG | | GMH | | GKGLEW | | SSKYYA | | ENSKNT | | YDPLYLN | | VTVSS |
| | | | VVQPG | | | | VA | | DSVKG | | LYLQMN | | H | | |
| | | | KSLRL | | | | | | | | SLRVED | | | | |
| | | | SCVAS | | | | | | | | TALYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81598 | 1286 | CAGGTGCAGCTGGTGCAGTCTGGGGGAGGCTTGGTACAGCC | | | | | | 6861 | QVQLVQSGGGLVQP | | 7214 | 16 | | 10 | |
| | | GGGGGGGTCCCTGAGACTCTCCTGTGCAGCCTCTGGATTCA | | | | | | | GGSLRLSCAASGFT | | | | | | |
| | | CCTTCAGTCGTTTTGACATGCACTGGTTCCGCCAAGGCACA | | | | | | | FSRFDMHWFRQGTG | | | | | | |
| | | GGGAAAGGTCTAGAGTGGGTCTCAGCTATTGGTACTTCTGG | | | | | | | KGLEWVSAIGTSGD | | | | | | |
| | | TGACACATATTATGGAGACTCCGTGAAGGGCCGATTCACGA | | | | | | | TYYGDSVKGRFTIS | | | | | | |
| | | TCAGCAGAGAAAATGCCAAGAACTCCTTGAATCTTCAAATG | | | | | | | RENAKNSLNLQMNS | | | | | | |
| | | AACAGCCTGAGAGCCGAAGACACGGCTATATATTACTGTGC | | | | | | | LRAEDTAIYYCARA | | | | | | |
| | | AAGAGCGACTCCGCATAGTAGTGGTTATTGGGACTGGTTCG | | | | | | | TPHSSGYWDWFDPW | | | | | | |
| | | ACCCCTGGGGCCAGGGAACCCTGGTCACCGTCTCCTCA | | | | | | | GQGTLVTVSS | | | | | | |
| ADI-81914 | 6807 | GAGGTGCAGCTGGTGGAGTCTGGGGGAGGCGTAGTCCAGCC | | | | | | 6862 | EVQLVESGGGVVQP | | 7215 | 16 | | 11 | |
| | | TGGGAGGTCCCTAAGACTCTCCTGTGCAGCCTCTGGATTCA | | | | | | | GRSLRLSCAASGFT | | | | | | |
| | | CCTTTGGAAACTATGCTATGCACTGGGTCCGCCAGGCTCCG | | | | | | | FGNYAMHWVRQAPD | | | | | | |
| | | GACAAGGGGCTTGAGTGGGTGGCAGTTGTCTCATATGATGG | | | | | | | KGLEWVAVVSYDGS | | | | | | |
| | | AAGTAATGAATTCTACGCAGACTCCGTGAAGGGCCGATTCA | | | | | | | NEFYADSVKGRFSI | | | | | | |
| | | GCATCTCCAGAGACAATTCCAAGAACACACTCCATCTGCAA | | | | | | | SRDNSKNTLHLQMN | | | | | | |
| | | ATGAACAGCCTGAAGGCTGAAGACACGGCTGAATATTTTTG | | | | | | | SLKAEDTAEYFCVR | | | | | | |
| | | TGTGAGACCCCACCTCCCTGATTACTTTGGTTCAGGGAGTT | | | | | | | PHLPDYFGSGSFHY | | | | | | |
| | | TTCACTACTGGGGCCAGGGAGTCCTGGTCACCGTCTCCTCA | | | | | | | WGQGVLVTVSS | | | | | | |
| ADI-81637 | 1286 | TGGGTCCTCGGTGAAGGTCTCCTGCAAGGCTTCTGGGGGCG | | | | | | 6863 | EVQLVQSGPAVKKP | | 7216 | 15 | | 15 | |
| | | CCTTCAGCATCCATGGTATCACCTGGGTGCGCCAGGCCCCT | | | | | | | GSSVKVSCKASGGA | | | | | | |
| | | GGACAAGGGCTTGAGTGGCTGGGAGGGATCATCCCTATGTT | | | | | | | FSIHGITWVRQAPG | | | | | | |
| | | TGGAACAGCAAACTACCCACAGAACTTCCAGGGCAGAGTCA | | | | | | | QGLEWLGGIIPMFG | | | | | | |
| | | CGATTACCGCGGAGGAATCCACGAGCACAGTCTACATGGAG | | | | | | | TANYPQNFQGRVTI | | | | | | |
| | | CTGAACAGCCTGCGATGTGAGGACACGGCCATGTACTACTG | | | | | | | TAEESTSTVYMELN | | | | | | |
| | | TGCGAGGGATGGCAGATATTGTAACAGTACCGCCTGCCTTA | | | | | | | SLRCEDTAMYYCAR | | | | | | |
| | | ACTTCTTTGACTTATGGGGCCAGGGAACCCTGGTCACTGTC | | | | | | | DGRYCNSTACLNFF | | | | | | |
| | | TCCTCA | | | | | | | DLWGQGTLVTVSS | | | | | | |
| ADI-81641 | 1286 | GAGGTGCAGCTGTTGGAGTCGGCCCCGGGACTGGTGAAGCC | | | | | | 6864 | EVQLLESAPGLVKP | | 7217 | 14 | | 10 | |
| | | TTCGGGGACCCTGTCCCTCACCTGCGCTGTCTCTGGTGACT | | | | | | | SGTLSLTCAVSGDS | | | | | | |
| | | CCATCAGCAGTGGTTACTGGTGGAGTTGGGTCCGCCAGCCC | | | | | | | ISSGYWWSWVRQPP | | | | | | |
| | | CCACTGAAGGGGCTGGAGTGGATTGGGGACATCTATCATAG | | | | | | | LKGLEWIGDIYHSG | | | | | | |
| | | TGGCAACACCGTCTACAACCCGTCCCTCAAGAGCCGAGTCA | | | | | | | NTVYNPSLKSRVTI | | | | | | |
| | | CCATTTCAGTAGACAAGTCCAAGAACCACTTCTCCCTGAGT | | | | | | | SVDKSKNHFSLSLK | | | | | | |
| | | CTGAAGTCTGTGACCGCCGCGGACACGGCCGTCTATTACTG | | | | | | | SVTAADTAVYYCAR | | | | | | |
| | | TGCGCGATCAATAGGATCTCGTCCGAGGTGGTTCGACCCCT | | | | | | | SIGSRPRWFDPWGQ | | | | | | |
| | | GGGGCCAGGGAACCCTGGTCACCGTCTCCTCA | | | | | | | GTLVTVSS | | | | | | |
| ADI-81729 | 1286 | GAGGTGCAGCTGTTGGAGTCTGGGACTGAGGTGAAGAAGCC | | | | | | 6865 | EVQLLESGTEVKKP | | 7218 | 14 | | 11 | |
| | | TGGGGCCTCAGTGAAGGTTTCCTGCAAGGCACTTGGATACA | | | | | | | GASVKVSCKALGYT | | | | | | |
| | | CTTTCACCAATAACTACATACACTGGGTGCGACAGGCCCCT | | | | | | | FTNNYIHWVRQAPG | | | | | | |
| | | GGACAAGGGCTTGAGTGGATGGGAATAATCAATCCTAGTGG | | | | | | | QGLEWMGIINPSGG | | | | | | |
| | | TGGCGCCACAACCTACGCACAGAAGTTCCAGGGTAGAGTCA | | | | | | | ATTYAQKFQGRVTL | | | | | | |
| | | CCTTGACCAGCGACACATCCACGAGCACAGTCTACCTGGAG | | | | | | | TSDTSTSTVYLELT | | | | | | |
| | | CTGACCAGCCTGACATCTGAGGACACGGCCGTCTATTACTG | | | | | | | SLTSEDTAVYYCAR | | | | | | |
| | | TGCGAGAGATAGCGAATATATACCAGCAGCTGGTTCTTTTG | | | | | | | DSEYIPAAGSFDYW | | | | | | |
| | | ACTACTGGGGCCAGGGAACCCTGGTCACCGTCTCCTCA | | | | | | | GQGTLVTVSS | | | | | | |
| ADI-81643 | 1287 | CGGGGAGTCTCTGACGATCTCCTGTAAGGCTTCTGGATACA | | | | | | 6866 | EVQLVESGPEVKKP | | 7219 | 11 | | 9 | |
| | | GCTTTATCAGCTATTGGATCACCTGGGTGCGCCAGATGCCC | | | | | | | GESLTISCKASGYS | | | | | | |
| | | GGGAAAGGCCTGGAGTGGATGGGGAACATTGATCCTAGTGA | | | | | | | FISYWITWVRQMPG | | | | | | |
| | | CTCTTACACCAACTATAGTCCGGCCTTCCAAGGCCACGTCA | | | | | | | KGLEWMGNIDPSDS | | | | | | |
| | | CCATCGCAGCTGACAAGGGCATCAGCACTGCCTACCTACAG | | | | | | | YTNYSPAFQGHVTI | | | | | | |
| | | TGGAGGAGCCTGAAGGCCTCGGACACCGCCATGTATTATTG | | | | | | | AADKGISTAYLQWR | | | | | | |
| | | TGCGAGACGAATTATGATTACATCTGGTGGAAAAGAGATCG | | | | | | | SLKASDTAMYYCAR | | | | | | |
| | | ATGCTTTTGATATCTGGGGCCAAGGGACAACGGTCACCGTC | | | | | | | RIMITSGGKEIDAF | | | | | | |
| | | TCCTCA | | | | | | | DIWGQGTTVTVSS | | | | | | |
| ADI-81701 | 1286 | GAGGTCCAGCTGGTGCAGTCTGGAGCTGAGGTGAAGAAGCC | | | | | | 6867 | EVQLVQSGAEVKKP | | 7220 | 11 | | 11 | |
| | | TGGGGCCTCAGTGAAGGTCTCCTGCAAGACTTCTGGTTACG | | | | | | | GASVKVSCKTSGYA | | | | | | |
| | | CCTTTATCAACTATGGTATCAGCTGGGTGCGACAGGCCCCT | | | | | | | FINYGISWVRQAPG | | | | | | |
| | | GGACAAGGGCTTGAGTGGATGGGATGGATCAGCCCTTACGA | | | | | | | QGLEWMGWISPYDG | | | | | | |
| | | TGGCCACACAAACTATGCACAGAAAATCCAGGGCAGAGTCA | | | | | | | HTNYAQKIQGRVTM | | | | | | |
| | | CCATGGCCACAGACACATCCGCGAGCACAGCCTACATGGAC | | | | | | | ATDTSASTAYMDLR | | | | | | |
| | | CTGAGGAGCCTGAGATCTGACGACACGGCCGTCTATTACTG | | | | | | | SLRSDDTAVYYCVR | | | | | | |
| | | TGTGAGAGATTACTACGGTGCTTTGACAAGCCCAAATGCCA | | | | | | | DYYGALTSPNANSE | | | | | | |
| | | ATAGTGAGACGTTGGGAACGATGGACTACTGGGGCCAGGGA | | | | | | | TLGTMDYWGQGTLV | | | | | | |
| | | ACCCTGGTCACCGTCTCCTCA | | | | | | | TVSS | | | | | | |
| ADI-81720 | 1286 | GAGGTGCAGCTGGTGGAGTCTGGGGGAGGCGTGGTCCAGCC | | | | | | 6868 | EVQLVESGGGVVQP | | 7221 | 11 | | 10 | |
| | | TGGGAAGTCCCTGAGACTCTCCTGTGTAGCGTCTGGATTCA | | | | | | | GKSLRLSCVASGFT | | | | | | |
| | | CCTTCAGTAGACATGGCATGCACTGGGTCCGCCAGGCTCCA | | | | | | | FSRHGMHWVRQAPG | | | | | | |
| | | GGCAAGGGGCTGGAGTGGGTGGCACTTATATATTATGATGG | | | | | | | KGLEWVALIYYDGS | | | | | | |
| | | AAGTAGTAAATATTATGCAGACTCCGTGAAGGGCCGATTCA | | | | | | | SKYYADSVKGRFTI | | | | | | |
| | | CCATCTCCAGAGAGAATTCCAAGAACACGCTGTATCTGCAA | | | | | | | SRENSKNTLYLQMN | | | | | | |
| | | ATGAACAGCCTGAGAGTCGAGGACACGGCTCTGTATTACTG | | | | | | | SLRVEDTALYYCAR | | | | | | |
| | | TGCGAGAGATGGGGCTAATGGCTATGATCCTTTGTACCTCA | | | | | | | DGANGYDPLYLNHW | | | | | | |
| | | ACCACTGGGGCCAGGGCACCCTGGTCACCGTCTCCTCA | | | | | | | GQGTLVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81632 | 5 | IGHV4- | QVQLV | 5345 | DSISSS | 5544 | WIRQPP | 445 | SIFYSG | 5902 | RVTISV | 6209 | ARELGFS | 6511 | WGQGTL |
| | | 39 | ESGPG | | NSYWA | | GKGLEW | | STYYNP | | DTSKNQ | | DS | | VTVSS |
| | | | LVKPS | | | | IG | | SLKS | | FSLQLT | | | | |
| | | | ETLSL | | | | | | | | SVTATD | | | | |
| | | | TCTVS | | | | | | | | TAVYYC | | | | |
| | | | D | | | | | | | | | | | | |
| ADI-81859 | 37 | IGHV4- | EVQLL | 5346 | DSISRG | 5545 | WIRQHP | 5788 | FIYYSG | 5903 | RVTISV | 6210 | ARDFGGN | 6512 | WGPGTL |
| | | 31 | ESGPG | | GYYWS | | GRGLEW | | STYYNP | | DTSKNQ | | AQYFAF | | VTVSS |
| | | | LVKPS | | | | IG | | SLKS | | FSLKLS | | | | |
| | | | QTLSL | | | | | | | | SLTAAD | | | | |
| | | | TCTVS | | | | | | | | TAVYYC | | | | |
| | | | G | | | | | | | | | | | | |
| ADI-81891 | 67 | IGHV3- | EVQLV | 5338 | FTFRRY | 204 | WVRQAP | 431 | VILYDG | 542 | RFTISR | 777 | AKSQGTY | 6513 | WGQGTL |
| | | 30 | ESGGG | | GMH | | GKGLEW | | SNKDYA | | DNSKNT | | CSGGNCY | | VTVSS |
| | | | VVQPG | | | | VA | | DSVKG | | LYLQMN | | SNYFDY | | |
| | | | RSLRL | | | | | | | | SLRAED | | | | |
| | | | SCAAS | | | | | | | | TAVYYC | | | | |
| | | | G | | | | | | | | | | | | |
| ADI-81814 | 88 | IGHV4- | EVQLV | 5347 | GSISGS | 5546 | WVRQSP | 438 | EIFHSG | 5904 | RVTMSL | 6211 | LLFLFLA | 6514 | RAEVQL |
| | | 4 | ESGPG | | NWWA | | GKGLEW | | STVYNP | | DKSKSQ | | SS | | FTVSS |
| | | | LVKPS | | | | IG | | SLKS | | FSLNLI | | | | |
| | | | GTLSL | | | | | | | | TRSKMK | | | | |
| | | | TCAVS | | | | | | | | WVTFIS | | | | |
| | | | G | | | | | | | | | | | | |
| ADI-81602 | 60 | IGHV3- | PGARC | 5348 | FTFSNY | 396 | WVRQAP | 431 | LISYDG | 5905 | RFTISR | 777 | ARGGDCS | 6515 | WGQGTT |
| | | 30 | CSLGD | | AMH | | GKGLEW | | SNNYYA | | DNSKNT | | GGGCPYP | | VTVSS |
| | | | VWSSW | | | | VA | | DSVKG | | LYLQMN | | YYYNGID | | |
| | | | EVPAT | | | | | | | | SLRAED | | DV | | |
| | | | LLCSS | | | | | | | | TAVYYC | | | | |
| | | | G | | | | | | | | | | | | |
| ADI-81572 | 27 | IGHV1- | QVQLQ | 5349 | GTFSSS | 5547 | WIRQAP | 492 | TIIPIL | 5906 | RLTLTA | 6212 | ARESGYS | 6516 | WGRGTL |
| | | 69 | QSGAE | | PIS | | GQGLEW | | GVPNYA | | DKSTST | | APGSVTY | | VTVSS |
| | | | VKEPG | | | | MG | | QQFQA | | AYMELS | | FDL | | |
| | | | SSVKV | | | | | | | | SLRSED | | | | |
| | | | SCMAS | | | | | | | | TASYYC | | | | |
| | | | G | | | | | | | | | | | | |
| ADI-81871 | 48 | IGHV3- | EVQLV | 5350 | FTFSNY | 5548 | WVRQTP | 5778 | LMSDDG | 5907 | RFTISR | 6213 | ARDLPSS | 6517 | WGQGAL |
| | | 30 | QSGGG | | AFY | | GKGLEW | | AYKDYA | | DNSKNT | | SWSSTAF | | VTVSS |
| | | | VVQPG | | | | VA | | DSVKG | | LYLQMN | | DY | | |
| | | | RSLRL | | | | | | | | GLKSED | | | | |
| | | | SCAAS | | | | | | | | TAVYFC | | | | |
| | | | G | | | | | | | | | | | | |
| ADI-81560 | 26 | IGHV1- | QVQLV | 5351 | FNFDLS | 5549 | WVRQAR | 5789 | WIAVGS | 5908 | RVTITR | 6214 | AAPHCNR | 6518 | WGQGTT |
| | | 58 | QSGPE | | AVQ | | GQRLEW | | GKTDYA | | DMSINT | | TNCHDGF | | VTVSS |
| | | | VKKPG | | | | IG | | QKFHE | | AYLELS | | DL | | |
| | | | TSVKV | | | | | | | | SLRSED | | | | |
| | | | PCKAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81632 | 1286 | CAGGTGCAGCTGGTGGAGTCGGGCCCAGGACTGGTGAAGCC | | | | | | 6869 | QVQLVESGPGLVKP | | 7222 | 10 | | 9 | |
| | | TTCGGAGACCCTGTCCCTCACCTGCACTGTCTCTGATGACT | | | | | | | SETLSLTCTVSDDS | | | | | | |
| | | CCATCAGCAGTAGTAATTCCTACTGGGCCTGGATCCGCCAG | | | | | | | ISSSNSYWAWIRQP | | | | | | |
| | | CCCCCAGGGAAGGGGCTGGAGTGGATTGGGAGTATCTTTTA | | | | | | | PGKGLEWIGSIFYS | | | | | | |
| | | TAGTGGGAGCACCTACTACAACCCGTCCCTCAAGAGTCGAG | | | | | | | GSTYYNPSLKSRVT | | | | | | |
| | | TCACCATATCCGTAGACACGTCCAAGAACCAGTTCTCCCTG | | | | | | | ISVDTSKNQFSLQL | | | | | | |
| | | CAGTTAACTTCTGTGACCGCCACAGACACGGCTGTTTATTA | | | | | | | TSVTATDTAVYYCA | | | | | | |
| | | CTGTGCGAGAGAACTTGGATTTAGTGACTCCTGGGGCCAGG | | | | | | | RELGFSDSWGQGTL | | | | | | |
| | | GAACCCTGGTCACCGTCTCCTCA | | | | | | | VTVSS | | | | | | |
| ADI-81859 | 1302 | GAGGTGCAGCTGTTGGAGTCGGGCCCAGGACTGGTGAAGCC | | | | | | 6870 | EVQLLESGPGLVKP | | 7223 | 6 | | 6 | |
| | | TTCACAGACCCTGTCCCTCACCTGCACTGTCTCTGGTGACT | | | | | | | SQTLSLTCTVSGDS | | | | | | |
| | | CCATCAGCCGTGGTGGTTACTACTGGAGCTGGATCCGCCAG | | | | | | | ISRGGYYWSWIRQH | | | | | | |
| | | CACCCAGGGAGGGGCCTGGAGTGGATTGGGTTCATCTATTA | | | | | | | PGRGLEWIGFIYYS | | | | | | |
| | | CAGTGGGAGCACCTACTATAACCCGTCCCTCAAGAGTCGAG | | | | | | | GSTYYNPSLKSRVT | | | | | | |
| | | TCACCATATCAGTGGACACGTCTAAGAACCAGTTCTCCCTG | | | | | | | ISVDTSKNQFSLKL | | | | | | |
| | | AAGCTGAGCTCTCTGACTGCCGCGGACACGGCCGTATATTA | | | | | | | SSLTAADTAVYYCA | | | | | | |
| | | CTGTGCGAGAGACTTCGGTGGTAACGCCCAATACTTTGCCT | | | | | | | RDFGGNAQYFAFWG | | | | | | |
| | | TCTGGGGCCCGGGAACCCTGGTCACCGTCTCCTCA | | | | | | | PGTLVTVSS | | | | | | |
| ADI-81891 | 1286 | GAGGTGCAGCTGGTGGAGTCTGGGGGAGGCGTGGTCCAGCC | | | | | | 6871 | EVQLVESGGGVVQP | | 7224 | 4 | | 4 | |
| | | TGGGAGGTCCCTTAGACTCTCCTGTGCAGCCTCCGGATTCA | | | | | | | GRSLRLSCAASGFT | | | | | | |
| | | CCTTCAGGAGATATGGCATGCATTGGGTCCGCCAGGCTCCA | | | | | | | FRRYGMHWVRQAPG | | | | | | |
| | | GGCAAGGGGCTGGAGTGGGTGGCAGTTATATTATATGATGG | | | | | | | KGLEWVAVILYDGS | | | | | | |
| | | AAGTAATAAAGACTACGCAGACTCCGTGAAGGGCCGATTCA | | | | | | | NKDYADSVKGRFTI | | | | | | |
| | | CCATCTCCAGAGACAATTCCAAGAACACGCTGTATCTGCAA | | | | | | | SRDNSKNTLYLQMN | | | | | | |
| | | ATGAACAGCCTGAGAGCTGAAGACACGGCTGTGTATTACTG | | | | | | | SLRAEDTAVYYCAK | | | | | | |
| | | TGCGAAATCCCAGGGAACTTATTGCAGTGGGGGTAACTGCT | | | | | | | SQGTYCSGGNCYSN | | | | | | |
| | | ACTCGAATTACTTTGACTACTGGGGCCAGGGAACCCTGGTC | | | | | | | YFDYWGQGTLVTVS | | | | | | |
| | | ACCGTCTCCTCA | | | | | | | S | | | | | | |
| ADI-81814 | 6808 | GAGGTGCAGCTGGTGGAGTCGGGCCCAGGACTGGTGAAGCC | | | | | | 6872 | EVQLVESGPGLVKP | | 7225 | 38 | | 22 | |
| | | TTCGGGGACCCTGTCCCTCACCTGCGCTGTCTCCGGTGGCT | | | | | | | SGTLSLTCAVSGGS | | | | | | |
| | | CCATCAGCGGTAGTAACTGGTGGGCTTGGGTCCGCCAGTCC | | | | | | | ISGSNWWAWVRQSP | | | | | | |
| | | CCAGGGAAGGGACTGGAGTGGATTGGGGAAATCTTTCATAG | | | | | | | GKGLEWIGEIFHSG | | | | | | |
| | | TGGGAGCACCGTCTACAACCCGTCCCTCAAGAGTCGAGTCA | | | | | | | STVYNPSLKSRVTM | | | | | | |
| | | CCATGTCACTAGACAAGTCCAAGAGCCAGTTCTCCCTGAAC | | | | | | | SLDKSKSQFSLNLI | | | | | | |
| | | TTGATAACGCGTTCAAAAATGAAATGGGTTACGTTTATATC | | | | | | | TRSKMKWVTFISLL | | | | | | |
| | | TCTATTGTTCTTATTTCTGGCGTCCTCGAGGGCCGAGGTGC | | | | | | | FLFLASSRAEVQLF | | | | | | |
| | | AGCTGTTCACCGTCTCCTCA | | | | | | | TVSS | | | | | | |
| ADI-81602 | 1287 | CCAGGTGCCCGCTGTTGCAGTCTGGGGGACGTGTGGTCCAG | | | | | | 6873 | PGARCCSLGDVWSS | | 7226 | 36 | | 17 | |
| | | CTGGGAGGTCCCTGCGACTCTCCTGTGCAGCTCTGGATTCA | | | | | | | WEVPATLLCSSGFT | | | | | | |
| | | CCTTCAGTAACTATGCTATGCACTGGGTCCGCCAGGCTCCA | | | | | | | FSNYAMHWVRQAPG | | | | | | |
| | | GGCAAGGGGCTGGAGTGGGTGGCACTTATATCATATGATGG | | | | | | | KGLEWVALISYDGS | | | | | | |
| | | AAGCAATAATTACTACGCAGACTCCGTGAAGGGCCGATTCA | | | | | | | NNYYADSVKGRFTI | | | | | | |
| | | CCATCTCCAGAGACAATTCCAAGAACACGCTGTATCTGCAA | | | | | | | SRDNSKNTLYLQMN | | | | | | |
| | | ATGAACAGCCTGAGAGCTGAGGACACGGCTGTGTATTACTG | | | | | | | SLRAEDTAVYYCAR | | | | | | |
| | | TGCGAGAGGAGGTGATTGTAGTGGTGGTGGGTGCCCGTACC | | | | | | | GGDCSGGGCPYPYY | | | | | | |
| | | CCTACTACTACAATGGTATAGACGACGTCTGGGGCCAAGGG | | | | | | | YNGIDDVWGQGTTV | | | | | | |
| | | ACCACGGTCACCGTCTCCTCA | | | | | | | TVSS | | | | | | |
| ADI-81572 | 1288 | TGGGTCCTCGGTGAAGGTCTCCTGCATGGCTTCTGGAGGCA | | | | | | 6874 | QVQLQQSGAEVKEP | | 7227 | 17 | | 13 | |
| | | CCTTCAGCAGCTCTCCTATCAGCTGGATACGCCAGGCCCCT | | | | | | | GSSVKVSCMASGGT | | | | | | |
| | | GGACAAGGGCTTGAGTGGATGGGCACGATCATCCCTATCCT | | | | | | | FSSSPISWIRQAPG | | | | | | |
| | | TGGTGTACCAAACTACGCACAACAGTTCCAGGCCAGACTTA | | | | | | | QGLEWMGTIIPILG | | | | | | |
| | | CCCTTACCGCGGACAAATCCACGAGCACAGCCTACATGGAA | | | | | | | VPNYAQQFQARLTL | | | | | | |
| | | CTGAGCAGCCTGAGATCTGAGGACACGGCCTCTTATTACTG | | | | | | | TADKSTSTAYMELS | | | | | | |
| | | TGCGAGAGAGTCGGGTTACTCTGCTCCGGGGAGTGTTACCT | | | | | | | SLRSEDTASYYCAR | | | | | | |
| | | ACTTCGATCTCTGGGGCCGTGGCACCCTGGTCACCGTCTCC | | | | | | | ESGYSAPGSVTYFD | | | | | | |
| | | TCA | | | | | | | LWGRGTLVTVSS | | | | | | |
| ADI-81871 | 1285 | GAGGTGCAGCTGGTGCAGTCTGGGGGAGGCGTGGTCCAGCC | | | | | | 6875 | EVQLVQSGGGVVQP | | 7228 | 17 | | 14 | |
| | | TGGGAGGTCCCTGAGACTCTCCTGTGCAGCCTCTGGATTCA | | | | | | | GRSLRLSCAASGFT | | | | | | |
| | | CCTTCAGTAACTATGCTTTCTACTGGGTCCGCCAGACTCCA | | | | | | | FSNYAFYWVRQTPG | | | | | | |
| | | GGCAAGGGGCTAGAGTGGGTGGCACTTATGTCAGATGATGG | | | | | | | KGLEWVALMSDDGA | | | | | | |
| | | AGCTTATAAAGACTACGCAGACTCCGTGAAGGGCCGATTCA | | | | | | | YKDYADSVKGRFTI | | | | | | |
| | | CCATCTCCAGAGACAATTCCAAGAACACGCTGTATCTGCAA | | | | | | | SRDNSKNTLYLQMN | | | | | | |
| | | ATGAACGGCCTGAAAAGTGAGGACACGGCTGTTTATTTCTG | | | | | | | GLKSEDTAVYFCAR | | | | | | |
| | | TGCGAGAGATCTGCCAAGCAGCAGCTGGTCATCAACCGCCT | | | | | | | DLPSSSWSSTAFDY | | | | | | |
| | | TTGACTACTGGGGCCAGGGAGCCCTGGTCACCGTCTCCTCA | | | | | | | WGQGALVTVSS | | | | | | |
| ADI-81560 | 1287 | CAGGTCCAGCTGGTGCAGTCTGGGCCTGAGGTGAAGAAGCC | | | | | | 6876 | QVQLVQSGPEVKKP | | 7229 | 14 | | 11 | |
| | | TGGGACCTCAGTTAAGGTCCCCTGCAAGGCTTCTGGTTTCA | | | | | | | GTSVKVPCKASGFN | | | | | | |
| | | ACTTTGATCTCTCTGCTGTGCAGTGGGTGCGACAGGCTCGT | | | | | | | FDLSAVQWVRQARG | | | | | | |
| | | GGACAACGCCTTGAGTGGATAGGGTGGATCGCCGTTGGCAG | | | | | | | QRLEWIGWIAVGSG | | | | | | |
| | | TGGCAAAACAGACTACGCACAGAAGTTCCATGAAAGAGTCA | | | | | | | KTDYAQKFHERVTI | | | | | | |
| | | CCATAACCAGGGACATGTCAATAAATACGGCCTACCTGGAG | | | | | | | TRDMSINTAYLELS | | | | | | |
| | | CTGAGCAGCCTGAGATCCGAGGACACGGCCGTCTATTACTG | | | | | | | SLRSEDTAVYYCAA | | | | | | |
| | | TGCGGCACCCCATTGCAATCGTACCAACTGCCACGATGGTT | | | | | | | PHCNRTNCHDGFDL | | | | | | |
| | | TTGATCTCTGGGGCCAAGGGACCACGGTCACTGTCTCCTCA | | | | | | | WGQGTTVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81802 | 78 | IGHV3- | EVQLV | 5338 | FPFSTY | 5550 | WVRQAP | 5790 | VISYDG | 5909 | RFTVSR | 6215 | ASRSPRV | 6519 | WGQGTT |
| | | 30 | ESGGG | | PMH | | GRGLEW | | AHKFYA | | DNSKNT | | PATATNA | | VTVSS |
| | | | VVQPG | | | | VA | | DSVKG | | LYLQLN | | FDI | | |
| | | | RSLRL | | | | | | | | SLRPED | | | | |
| | | | SCAAS | | | | | | | | TAVYSC | | | | |
| | | | G | | | | | | | | | | | | |
| ADI-81558 | 24 | IGHV3- | QVQLV | 5352 | FPFSNA | 5551 | WVRQAP | 5791 | RIKNGP | 5910 | RFTISR | 6216 | TTMSREY | 6520 | WGQGTT |
| | | 15 | ESGGS | | WMT | | GKGLEW | | DGGTTD | | DDSKNT | | YDTDFYA | | VTVSS |
| | | | LVKPG | | | | IG | | YAAPVR | | LYLQMN | | KDV | | |
| | | | GSLRL | | | | | | G | | SLKSED | | | | |
| | | | SCAAS | | | | | | | | TGVYFC | | | | |
| | | | G | | | | | | | | | | | | |
| ADI-81695 | 65 | IGHV3- | EVQLV | 5321 | FTFEDY | 5552 | WVRQAP | 437 | GTAWNS | 5911 | RFFISR | 6217 | VKVFSRD | 6521 | WGQGTT |
| | | 9 | ESGGG | | GMH | | GKGLEW | | ATIGYA | | DNAKSS | | WSGYAFD | | VTVSS |
| | | | LVQPG | | | | VS | | DSVKG | | LYLQMI | | M | | |
| | | | RSLRL | | | | | | | | SLRAED | | | | |
| | | | SCAAS | | | | | | | | TALYYC | | | | |
| | | | G | | | | | | | | | | | | |
| ADI-81719 | 89 | IGHV3- | EVQLL | 5353 | FPFITY | 5553 | WVRQAP | 437 | HISSGG | 5912 | RFTISR | 6218 | ARVLRYS | 6522 | WGQGTL |
| | | 48 | ESGGG | | EMN | | GKGLEW | | TTTYYA | | DNDENS | | SAWVDL | | VTVSS |
| | | | LVQPG | | | | VS | | DSVKG | | LVLQMN | | | | |
| | | | GSLRL | | | | | | | | SLRAED | | | | |
| | | | SCAAT | | | | | | | | TAVYYC | | | | |
| | | | G | | | | | | | | | | | | |
| ADI-81758 | 34 | IGHV3- | EVQLL | 5354 | IIVSRN | 291 | WVRQAP | 5792 | VIYPGG | 622 | RFTISR | 6219 | ARQENND | 6523 | WGQGTT |
| | | 53 | ETGGG | | YMT | | GKGLDW | | STFYAD | | DDSKNT | | AFDI | | VTVSS |
| | | | LIQPG | | | | VS | | SVRG | | LYLQMI | | | | |
| | | | GSLRL | | | | | | | | SLRAED | | | | |
| | | | SCAAS | | | | | | | | TAVYYC | | | | |
| | | | E | | | | | | | | | | | | |
| ADI-81780 | 56 | IGHV3- | EVQLL | 5355 | FIFTNY | 5554 | WVRQAP | 431 | TVKQDG | 5913 | RFTISR | 6220 | ARELGIL | 6524 | WGQGTL |
| | | 7 | ESGGG | | WIS | | GKGLEW | | TEKYYV | | DNAKNS | | DW | | VTVSS |
| | | | LVQPG | | | | VA | | DSVKG | | VFLQMN | | | | |
| | | | GSLRL | | | | | | | | SLRAED | | | | |
| | | | SCAAS | | | | | | | | TAVYYC | | | | |
| | | | G | | | | | | | | | | | | |
| ADI-81574 | 36 | IGHV3- | QVQLV | 12 | FTFSTS | 5555 | WVRQVT | 5793 | GVGTSG | 5914 | RFTISR | 6221 | ARGETTM | 6525 | WGQGTT |
| | | 13 | QSGGG | | DMH | | GKHLEW | | DTYYSD | | ENAKNS | | ALRWYAF | | VTVSS |
| | | | LVQPG | | | | VS | | SVKG | | LYLQMN | | DI | | |
| | | | GSLRL | | | | | | | | NLRAGD | | | | |
| | | | SCAAS | | | | | | | | TAVYYC | | | | |
| | | | G | | | | | | | | | | | | |
| ADI-81698 | 68 | IGHV1- | QVQLV | 5356 | YTFTSY | 5556 | WVRQAP | 433 | IINPGD | 5915 | RVTVTS | 6222 | ARDFLPA | 6526 | WGKGTT |
| | | 46 | QSGAE | | FIH | | GQGLEW | | DSTTYS | | DTSTST | | TASPYYY | | VTVSS |
| | | | VKKPG | | | | MG | | QKFQG | | VYMELS | | YYMDV | | |
| | | | ASVKL | | | | | | | | SLRSED | | | | |
| | | | SCKAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81802 | 1287 | TGGGAGGTCCCTGAGACTCTCCTGTGCAGCCTCTGGATTCC | | | | | | 6877 | EVQLVESGGGVVQP | | 7230 | 14 | | 11 | |
| | | CCTTCAGTACCTATCCTATGCACTGGGTCCGCCAGGCTCCA | | | | | | | GRSLRLSCAASGFP | | | | | | |
| | | GGCAGGGGGCTGGAATGGGTGGCAGTTATTTCATATGATGG | | | | | | | FSTYPMHWVRQAPG | | | | | | |
| | | AGCCCATAAATTCTACGCAGACTCCGTGAAGGGCCGATTCA | | | | | | | RGLEWVAVISYDGA | | | | | | |
| | | CCGTCTCCAGAGACAATTCCAAGAACACACTGTATCTACAA | | | | | | | HKFYADSVKGRFTV | | | | | | |
| | | CTGAACAGCCTGAGACCTGAGGACACGGCTGTTTATTCTTG | | | | | | | SRDNSKNTLYLQLN | | | | | | |
| | | TGCGAGTCGCTCACCGCGTGTACCAGCAACTGCTACTAATG | | | | | | | SLRPEDTAVYSCAS | | | | | | |
| | | CTTTTGATATCTGGGGCCAAGGGACCACGGTCACCGTCTCC | | | | | | | RSPRVPATATNAFD | | | | | | |
| | | TCA | | | | | | | IWGQGTTVTVSS | | | | | | |
| ADI-81558 | 1287 | TGGGGGGTCCCTTAGACTCTCCTGTGCAGCCTCTGGATTCC | | | | | | 6878 | QVQLVESGGSLVKP | | 7231 | 12 | | 11 | |
| | | CTTTCAGTAACGCCTGGATGACCTGGGTCCGCCAGGCTCCA | | | | | | | GGSLRLSCAASGFP | | | | | | |
| | | GGGAAGGGGCTGGAGTGGATTGGCCGTATTAAGAACGGACC | | | | | | | FSNAWMTWVRQAPG | | | | | | |
| | | TGATGGTGGGACAACAGACTACGCTGCACCCGTGAGAGGCA | | | | | | | KGLEWIGRIKNGPD | | | | | | |
| | | GATTCACCATCTCAAGAGATGATTCAAAAAACACGCTGTAT | | | | | | | GGTTDYAAPVRGRF | | | | | | |
| | | CTTCAAATGAACAGCCTGAAAAGCGAGGACACAGGCGTTTA | | | | | | | TISRDDSKNTLYLQ | | | | | | |
| | | TTTCTGTACCACAATGAGCCGAGAATACTATGATACCGACT | | | | | | | MNSLKSEDTGVYFC | | | | | | |
| | | TCTACGCTAAGGACGTCTGGGGCCAAGGGACCACGGTCACC | | | | | | | TTMSREYYDTDFYA | | | | | | |
| | | GTCTCCTCA | | | | | | | KDVWGQGTTVTVSS | | | | | | |
| ADI-81695 | 1287 | GAGGTGCAGCTGGTGGAGTCTGGGGGAGGTTTGGTACAGCC | | | | | | 6879 | EVQLVESGGGLVQP | | 7232 | 12 | | 9 | |
| | | TGGCAGGTCCCTGAGACTCTCTTGTGCAGCCTCTGGATTCA | | | | | | | GRSLRLSCAASGFT | | | | | | |
| | | CCTTTGAAGATTATGGCATGCACTGGGTCCGGCAAGCTCCT | | | | | | | FEDYGMHWVRQAPG | | | | | | |
| | | GGGAAGGGCCTGGAGTGGGTATCAGGCACTGCCTGGAATAG | | | | | | | KGLEWVSGTAWNSA | | | | | | |
| | | TGCTACCATAGGCTATGCGGACTCTGTGAAGGGCCGATTCT | | | | | | | TIGYADSVKGRFFI | | | | | | |
| | | TCATCTCCAGAGACAACGCCAAGAGCTCCCTGTATCTGCAA | | | | | | | SRDNAKSSLYLQMI | | | | | | |
| | | ATGATCAGTCTGAGAGCTGAGGACACGGCCTTGTATTACTG | | | | | | | SLRAEDTALYYCVK | | | | | | |
| | | TGTAAAAGTTTTTTCCCGGGATTGGAGTGGTTACGCTTTTG | | | | | | | VFSRDWSGYAFDMW | | | | | | |
| | | ATATGTGGGGCCAAGGGACCACGGTCACCGTCTCCTCA | | | | | | | GQGTTVTVSS | | | | | | |
| ADI-81719 | 1286 | GAGGTGCAGCTGTTGGAGTCGGGGGGAGGCTTGGTACAGCC | | | | | | 6880 | EVQLLESGGGLVQP | | 7233 | 12 | | 11 | |
| | | TGGAGGGTCCCTGAGACTCTCCTGTGCAGCCACTGGATTCC | | | | | | | GGSLRLSCAATGFP | | | | | | |
| | | CCTTCATTACTTATGAAATGAACTGGGTCCGCCAGGCTCCA | | | | | | | FITYEMNWVRQAPG | | | | | | |
| | | GGGAAGGGGCTGGAGTGGGTCTCACACATTAGTAGTGGTGG | | | | | | | KGLEWVSHISSGGT | | | | | | |
| | | TACTACCACATACTACGCAGACTCTGTGAAGGGCCGATTCA | | | | | | | TTYYADSVKGRFTI | | | | | | |
| | | CCATCTCCAGAGACAACGACGAGAACTCACTGGTTTTGCAA | | | | | | | SRDNDENSLVLQMN | | | | | | |
| | | ATGAACAGCCTGAGAGCCGAGGACACGGCTGTTTATTACTG | | | | | | | SLRAEDTAVYYCAR | | | | | | |
| | | TGCGAGAGTCTTGCGTTATAGCAGTGCCTGGGTTGACCTCT | | | | | | | VLRYSSAWVDLWGQ | | | | | | |
| | | GGGGCCAGGGAACCCTGGTCACCGTCTCCTCA | | | | | | | GTLVTVSS | | | | | | |
| ADI-81758 | 1287 | GAGGTGCAGCTGTTGGAGACTGGAGGAGGCTTGATCCAGCC | | | | | | 6881 | EVQLLETGGGLIQP | | 7234 | 12 | | 11 | |
| | | GGGGGGGTCCCTGAGACTCTCCTGTGCAGCCTCTGAGATCA | | | | | | | GGSLRLSCAASEII | | | | | | |
| | | TCGTCAGCAGGAACTACATGACCTGGGTCCGCCAGGCTCCA | | | | | | | VSRNYMTWVRQAPG | | | | | | |
| | | GGGAAAGGGCTGGACTGGGTCTCAGTCATTTATCCCGGTGG | | | | | | | KGLDWVSVIYPGGS | | | | | | |
| | | GAGCACTTTCTACGCAGACTCCGTGAGGGGCCGATTCACCA | | | | | | | TFYADSVRGRFTIS | | | | | | |
| | | TCTCCAGAGACGATTCCAAGAACACTCTGTATCTTCAAATG | | | | | | | RDDSKNTLYLQMIS | | | | | | |
| | | ATCAGCCTGAGAGCCGAGGACACGGCCGTATATTACTGTGC | | | | | | | LRAEDTAVYYCARQ | | | | | | |
| | | GAGACAAGAAAATAATGATGCTTTTGATATTTGGGGGCAAG | | | | | | | ENNDAFDIWGQGTT | | | | | | |
| | | GGACCACGGTCACCGTCTCCTCA | | | | | | | VTVSS | | | | | | |
| ADI-81780 | 1286 | GAGGTGCAGCTGTTGGAGTCTGGGGGAGGCTTGGTCCAGCC | | | | | | 6882 | EVQLLESGGGLVQP | | 7235 | 12 | | 9 | |
| | | GGGGGGGTCCCTGAGACTCTCCTGTGCAGCCTCTGGATTCA | | | | | | | GGSLRLSCAASGFI | | | | | | |
| | | TTTTCACTAACTATTGGATAAGTTGGGTCCGCCAGGCTCCA | | | | | | | FTNYWISWVRQAPG | | | | | | |
| | | GGGAAGGGGCTGGAGTGGGTGGCCACCGTGAAGCAAGATGG | | | | | | | KGLEWVATVKQDGT | | | | | | |
| | | AACTGAGAAATATTATGTGGACTCTGTGAAGGGCCGATTCA | | | | | | | EKYYVDSVKGRFTI | | | | | | |
| | | CCATCTCCAGAGACAACGCCAAAAACTCAGTCTTTCTGCAA | | | | | | | SRDNAKNSVFLQMN | | | | | | |
| | | ATGAACAGCCTGAGAGCCGAGGACACGGCCGTGTATTATTG | | | | | | | SLRAEDTAVYYCAR | | | | | | |
| | | TGCGAGAGAATTGGGGATCTTGGATTGGTGGGGCCAGGGAA | | | | | | | ELGILDWWGQGTLV | | | | | | |
| | | CCCTGGTCACCGTCTCCTCA | | | | | | | TVSS | | | | | | |
| ADI-81574 | 1287 | CAGGTGCAGCTGGTGCAGTCTGGGGGAGGCTTGGTACAGCC | | | | | | 6883 | QVQLVQSGGGLVQP | | 7236 | 11 | | 10 | |
| | | TGGGGGGTCCCTGAGACTCTCCTGTGCAGCCTCTGGATTCA | | | | | | | GGSLRLSCAASGFT | | | | | | |
| | | CCTTCAGTACCTCCGACATGCACTGGGTCCGCCAAGTTACA | | | | | | | FSTSDMHWVRQVTG | | | | | | |
| | | GGAAAACATCTGGAATGGGTCTCAGGTGTTGGAACTTCTGG | | | | | | | KHLEWVSGVGTSGD | | | | | | |
| | | TGACACATACTATTCAGACTCCGTGAAGGGCCGATTCACCA | | | | | | | TYYSDSVKGRFTIS | | | | | | |
| | | TCTCCAGAGAAAATGCCAAGAACTCTTTATATCTTCAAATG | | | | | | | RENAKNSLYLQMNN | | | | | | |
| | | AACAACCTGAGAGCCGGGGACACGGCTGTGTATTACTGTGC | | | | | | | LRAGDTAVYYCARG | | | | | | |
| | | AAGAGGGGAAACAACTATGGCGCTCCGGTGGTATGCTTTTG | | | | | | | ETTMALRWYAFDIW | | | | | | |
| | | ATATCTGGGGCCAAGGGACCACGGTCACCGTCTCCTCA | | | | | | | GQGTTVTVSS | | | | | | |
| ADI-81698 | 1291 | TGGGGCCTCAGTGAAACTTTCCTGCAAGGCATCTGGATACA | | | | | | 6884 | QVQLVQSGAEVKKP | | 7237 | 11 | | 10 | |
| | | CCTTCACCAGTTACTTTATTCATTGGGTGCGACAGGCCCCT | | | | | | | GASVKLSCKASGYT | | | | | | |
| | | GGACAAGGGCTTGAGTGGATGGGAATAATCAACCCTGGTGA | | | | | | | FTSYFIHWVRQAPG | | | | | | |
| | | TGATAGCACAACTTACTCACAGAAGTTCCAGGGCAGAGTCA | | | | | | | QGLEWMGIINPGDD | | | | | | |
| | | CCGTGACCAGTGACACGTCCACGAGCACAGTCTACATGGAG | | | | | | | STTYSQKFQGRVTV | | | | | | |
| | | CTGAGCAGCCTGAGATCTGAAGACACGGCCGTGTATTACTG | | | | | | | TSDTSTSTVYMELS | | | | | | |
| | | TGCGAGAGATTTCCTCCCAGCGACCGCGTCCCCCTACTACT | | | | | | | SLRSEDTAVYYCAR | | | | | | |
| | | ACTATTATATGGACGTCTGGGGCAAAGGGACCACGGTCACC | | | | | | | DFLPATASPYYYYY | | | | | | |
| | | GTCTCCTCA | | | | | | | MDVWGKGTTVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81831 | 10 | IGHV2- | QVTLK | 5357 | FSLNTG | 5557 | WLRQPP | 5794 | RIDWDY | 5916 | RLTIST | 6223 | ARFKYFY | 6527 | WGQGAL |
| | | 70 | ECGPA | | GICVS | | GKALEW | | EKHYSA | | DTSKNQ | | DRNNYYF | | VTVSS |
| | | | LVKPT | | | | LA | | SLTT | | VVLTMT | | GSGFDY | | |
| | | | QTLTL | | | | | | | | NMDPVD | | | | |
| | | | TCTFS | | | | | | | | TATYYC | | | | |
| | | | G | | | | | | | | | | | | |
| ADI-81712 | 82 | IGHV1- | EVQLV | 5358 | GTARTY | 5558 | WVRQAP | 433 | GIIPKY | 5917 | RVTITA | 6224 | AHSPGYS | 6528 | WGQGTL |
| | | 69 | QSGAE | | AIS | | GQGLEW | | DTTNYA | | DKSTST | | SGWYRAG | | VTVSS |
| | | | VKKPG | | | | MG | | QKFQG | | AYMELS | | ISWFDP | | |
| | | | SSVKV | | | | | | | | SLTSED | | | | |
| | | | SCKAS | | | | | | | | TAVYYC | | | | |
| | | | G | | | | | | | | | | | | |
| ADI-81746 | 22 | IGHV4- | QVQLL | 5359 | GSISSS | 5559 | WVRQPP | 444 | EIYHRG | 5918 | RVTISL | 6225 | ARAPAGD | 6529 | WGRGTL |
| | | 4 | ESGPG | | NWWT | | GKGLEW | | TTDYNP | | DKSKNY | | FRQLF | | VTVSS |
| | | | LVKPS | | | | IG | | SLKS | | FSLNLT | | | | |
| | | | GTLSL | | | | | | | | SVTAAD | | | | |
| | | | TCAVS | | | | | | | | TAVYYC | | | | |
| | | | G | | | | | | | | | | | | |
| ADI-81752 | 28 | IGHV5- | QVQLA | 5360 | YSFDTK | 5560 | WVRQMP | 5795 | TIYPGD | 665 | QVTISA | 914 | ARRADFS | 6530 | WGQGAL |
| | | 51 | QSGAE | | WIA | | GKGPEW | | SDTRYS | | DKSIST | | SSWTDYF | | VTVSS |
| | | | VKKPG | | | | LG | | PSFQG | | AYLQWS | | DY | | |
| | | | ESLKI | | | | | | | | SLKASD | | | | |
| | | | SCKGS | | | | | | | | TAIYYC | | | | |
| | | | G | | | | | | | | | | | | |
| ADI-81836 | 15 | IGHV2- | QVTLK | 5361 | FSLTTS | 5561 | WIRQPP | 441 | LVDWDD | 5919 | RLTISK | 6226 | ARIDRRR | 6531 | WGQGTT |
| | | 70 | ESGPA | | GMSVT | | GKALEW | | AKYYST | | DTSKNQ | | NCMDV | | VTVSS |
| | | | LVKPT | | | | LA | | SLTT | | VVLTMT | | | | |
| | | | QTLTL | | | | | | | | NMDPVD | | | | |
| | | | TCTFS | | | | | | | | TGTYYC | | | | |
| | | | G | | | | | | | | | | | | |
| ADI-81864 | 42 | IGHV3- | EVQLV | 5362 | VTVSRN | 5562 | WVRQAP | 437 | IIYSGG | 5920 | RFTISR | 6227 | VRRIVGG | 6532 | WGQGTT |
| | | 53 | ESGGG | | YMS | | GKGLEW | | SAYYAD | | DDSRNT | | NAGMDV | | VTVSS |
| | | | LIQPG | | | | VS | | SVKG | | LYLQMK | | | | |
| | | | GSLRL | | | | | | | | SLRAED | | | | |
| | | | SCAAS | | | | | | | | TAVYYC | | | | |
| | | | G | | | | | | | | | | | | |
| ADI-81722 | 92 | IGHV4- | EVQLL | 5346 | GSISGA | 5563 | WIRQHP | 5796 | HTEYSG | 5921 | LLNISI | 6228 | ARIHDYG | 6533 | WGQGTT |
| | | 31 | ESGPG | | PYYWT | | GRGLEW | | GTYYNP | | DRSKNQ | | GHISAFD | | VTVSS |
| | | | LVKPS | | | | MA | | SLKS | | FSLKVT | | M | | |
| | | | QTLSL | | | | | | | | SVTAAD | | | | |
| | | | TCTVS | | | | | | | | TAVYYC | | | | |
| | | | G | | | | | | | | | | | | |
| ADI-81595 | 55 | IGHV1- | QVQLQ | 86 | GTFSSY | 5564 | WVRQAP | 5797 | RILPLI | 5922 | RVTITA | 6229 | ARERGYS | 6534 | WGQGTL |
| | | 69 | QSGAE | | GLT | | GQGLEF | | GVPDYA | | DTSTGT | | GYGSAVY | | VTVSS |
| | | | VKKPG | | | | MG | | QSFQG | | AYVEVN | | FDI | | |
| | | | SSVKV | | | | | | | | SLRSED | | | | |
| | | | SCKAS | | | | | | | | TALYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81831 | 1285 | CAGGTCACGCTGAAGGAGTGTGGTCCTGCGCTGGTGAAACC | | | | | | 6885 | QVTLKECGPALVKP | | 7238 | 11 | | 10 | |
| | | CACACAGACCCTCACACTGACCTGCACCTTCTCTGGGTTCT | | | | | | | TQTLTLTCTFSGFS | | | | | | |
| | | CACTCAACACTGGAGGAATATGTGTGAGCTGGCTCCGTCAG | | | | | | | LNTGGICVSWLRQP | | | | | | |
| | | CCCCCAGGGAAGGCCCTGGAGTGGCTTGCACGCATTGATTG | | | | | | | PGKALEWLARIDWD | | | | | | |
| | | GGATTATGAGAAACACTATAGCGCATCTCTGACGACCAGAC | | | | | | | YEKHYSASLTTRLT | | | | | | |
| | | TCACCATCTCCACGGACACCTCCAAAAACCAGGTGGTCCTC | | | | | | | ISTDTSKNQVVLTM | | | | | | |
| | | ACAATGACCAACATGGACCCTGTGGACACAGCCACCTACTA | | | | | | | TNMDPVDTATYYCA | | | | | | |
| | | CTGTGCACGATTCAAGTATTTTTATGATAGGAATAATTACT | | | | | | | RFKYFYDRNNYYFG | | | | | | |
| | | ACTTCGGTTCGGGATTTGACTACTGGGGCCAGGGAGCCCTG | | | | | | | SGFDYWGQGALVTV | | | | | | |
| | | GTCACCGTCTCTTCA | | | | | | | SS | | | | | | |
| ADI-81712 | 1286 | GAGGTGCAGCTGGTGCAGTCTGGGGCTGAGGTGAAGAAGCC | | | | | | 6886 | EVQLVQSGAEVKKP | | 7239 | 10 | | 8 | |
| | | CGGGTCCTCGGTGAAGGTCTCCTGCAAGGCCTCTGGAGGCA | | | | | | | GSSVKVSCKASGGT | | | | | | |
| | | CCGCCAGGACCTATGCTATCAGTTGGGTGCGACAGGCCCCT | | | | | | | ARTYAISWVRQAPG | | | | | | |
| | | GGACAAGGGCTTGAGTGGATGGGAGGGATCATTCCTAAGTA | | | | | | | QGLEWMGGIIPKYD | | | | | | |
| | | TGATACAACAAACTACGCACAGAAGTTCCAGGGCAGAGTCA | | | | | | | TTNYAQKFQGRVTI | | | | | | |
| | | CGATTACCGCGGACAAATCTACGAGCACAGCCTACATGGAA | | | | | | | TADKSTSTAYMELS | | | | | | |
| | | CTGAGCAGCCTGACATCTGAGGACACGGCCGTGTATTATTG | | | | | | | SLTSEDTAVYYCAH | | | | | | |
| | | TGCGCACTCTCCCGGATATAGCAGTGGCTGGTATCGGGCCG | | | | | | | SPGYSSGWYRAGIS | | | | | | |
| | | GAATCAGCTGGTTCGACCCCTGGGGCCAGGGAACCCTGGTC | | | | | | | WFDPWGQGTLVTVS | | | | | | |
| | | ACCGTCTCCTCA | | | | | | | S | | | | | | |
| ADI-81746 | 1288 | CAGGTGCAGCTGTTGGAGTCGGGCCCGGGACTGGTGAAGCC | | | | | | 6887 | QVQLLESGPGLVKP | | 7240 | 10 | | 8 | |
| | | TTCGGGGACCCTGTCCCTCACCTGCGCTGTCTCTGGTGGCT | | | | | | | SGTLSLTCAVSGGS | | | | | | |
| | | CCATCAGCAGTAGTAACTGGTGGACTTGGGTCCGCCAGCCC | | | | | | | ISSSNWWTWVRQPP | | | | | | |
| | | CCAGGGAAGGGACTGGAGTGGATTGGGGAAATCTATCATAG | | | | | | | GKGLEWIGEIYHRG | | | | | | |
| | | AGGGACCACCGACTACAATCCGTCGTTGAAGAGTCGAGTCA | | | | | | | TTDYNPSLKSRVTI | | | | | | |
| | | CCATATCACTCGACAAGTCCAAGAACTACTTCTCCCTGAAT | | | | | | | SLDKSKNYFSLNLT | | | | | | |
| | | TTGACCTCTGTGACCGCCGCGGACACGGCCGTCTATTACTG | | | | | | | SVTAADTAVYYCAR | | | | | | |
| | | TGCGAGAGCCCCGGCGGGGGACTTTCGGCAGCTCTTCTGGG | | | | | | | APAGDFRQLFWGRG | | | | | | |
| | | GCCGGGGAACCCTGGTCACCGTCTCCTCA | | | | | | | TLVTVSS | | | | | | |
| ADI-81752 | 1285 | CAGGTGCAGCTGGCGCAGTCTGGAGCAGAGGTGAAAAAGCC | | | | | | 6888 | QVQLAQSGAEVKKP | | 7241 | 10 | | 8 | |
| | | CGGGGAGTCCCTGAAGATCTCCTGTAAGGGTTCTGGATACA | | | | | | | GESLKISCKGSGYS | | | | | | |
| | | GCTTTGACACCAAGTGGATCGCCTGGGTGCGCCAGATGCCC | | | | | | | FDTKWIAWVRQMPG | | | | | | |
| | | GGGAAAGGCCCGGAGTGGCTGGGGACCATTTATCCTGGTGA | | | | | | | KGPEWLGTIYPGDS | | | | | | |
| | | CTCTGATACCAGATACAGCCCGTCCTTCCAAGGCCAGGTCA | | | | | | | DTRYSPSFQGQVTI | | | | | | |
| | | CCATCTCAGCCGACAAGTCCATCAGCACCGCCTACCTGCAG | | | | | | | SADKSISTAYLQWS | | | | | | |
| | | TGGAGCAGCCTGAAGGCCTCGGACACCGCCATTTACTACTG | | | | | | | SLKASDTAIYYCAR | | | | | | |
| | | TGCGAGACGAGCGGACTTTAGCAGCAGCTGGACTGACTACT | | | | | | | RADFSSSWTDYFDY | | | | | | |
| | | TTGACTACTGGGGCCAGGGAGCCCTGGTCACCGTCTCCTCA | | | | | | | WGQGALVTVSS | | | | | | |
| ADI-81836 | 1287 | CAGGTCACCTTGAAGGAGTCTGGTCCTGCGCTGGTGAAACC | | | | | | 6889 | QVTLKESGPALVKP | | 7242 | 7 | | 7 | |
| | | CACACAGACCCTCACACTGACCTGCACCTTCTCTGGGTTCT | | | | | | | TQTLTLTCTFSGFS | | | | | | |
| | | CACTCACCACTAGTGGAATGTCTGTGACCTGGATCCGTCAG | | | | | | | LTTSGMSVTWIRQP | | | | | | |
| | | CCCCCAGGGAAGGCCCTGGAGTGGCTTGCACTCGTTGATTG | | | | | | | PGKALEWLALVDWD | | | | | | |
| | | GGATGATGCTAAATACTACAGCACGTCTCTGACGACCAGGC | | | | | | | DAKYYSTSLTTRLT | | | | | | |
| | | TCACCATCTCCAAGGACACCTCCAAAAACCAGGTGGTCCTA | | | | | | | ISKDTSKNQVVLTM | | | | | | |
| | | ACAATGACCAACATGGACCCTGTGGACACAGGCACCTATTA | | | | | | | TNMDPVDTGTYYCA | | | | | | |
| | | CTGTGCACGGATAGACCGACGCCGGAATTGTATGGACGTCT | | | | | | | RIDRRRNCMDVWGQ | | | | | | |
| | | GGGGCCAAGGGACCACGGTCACCGTCTCCTCA | | | | | | | GTTVTVSS | | | | | | |
| ADI-81864 | 1287 | GAGGTGCAGCTGGTGGAGTCTGGGGGAGGCTTGATCCAGCC | | | | | | 6890 | EVQLVESGGGLIQP | | 7243 | 7 | | 7 | |
| | | TGGGGGGTCCCTGAGACTCTCCTGTGCAGCCTCTGGGGTCA | | | | | | | GGSLRLSCAASGVT | | | | | | |
| | | CCGTCAGTAGGAACTACATGAGCTGGGTCCGCCAGGCTCCA | | | | | | | VSRNYMSWVRQAPG | | | | | | |
| | | GGGAAGGGGCTGGAGTGGGTCTCAATTATTTATAGCGGTGG | | | | | | | KGLEWVSIIYSGGS | | | | | | |
| | | TAGTGCATACTACGCAGACTCCGTGAAGGGCCGATTCACCA | | | | | | | AYYADSVKGRFTIS | | | | | | |
| | | TCTCCAGAGACGATTCCAGGAACACGCTGTATCTTCAAATG | | | | | | | RDDSRNTLYLQMKS | | | | | | |
| | | AAGAGCTTGAGAGCGGAAGACACGGCCGTGTATTATTGTGT | | | | | | | LRAEDTAVYYCVRR | | | | | | |
| | | GAGACGTATAGTGGGAGGCAATGCCGGCATGGACGTCTGGG | | | | | | | IVGGNAGMDVWGQG | | | | | | |
| | | GCCAAGGGACCACGGTCACCGTCTCCTCA | | | | | | | TTVTVSS | | | | | | |
| ADI-81722 | 1287 | GAGGTGCAGCTGTTGGAGTCGGGGCCAGGACTGGTGAAGCC | | | | | | 6891 | EVQLLESGPGLVKP | | 7244 | 23 | | 17 | |
| | | TTCGCAGACCCTGTCCCTCACCTGCACTGTCTCTGGTGGCT | | | | | | | SQTLSLTCTVSGGS | | | | | | |
| | | CCATCAGCGGCGCTCCTTACTACTGGACTTGGATCCGCCAG | | | | | | | ISGAPYYWTWIRQH | | | | | | |
| | | CACCCCGGGAGGGGCCTGGAGTGGATGGCCCACACCGAGTA | | | | | | | PGRGLEWMAHTEYS | | | | | | |
| | | CAGTGGGGGCACCTACTACAACCCGTCCCTTAAGAGTCTAC | | | | | | | GGTYYNPSLKSLLN | | | | | | |
| | | TTAACATATCAATAGACAGGTCTAAGAACCAGTTCTCCCTG | | | | | | | ISIDRSKNQFSLKV | | | | | | |
| | | AAGGTAACCTCTGTGACTGCCGCGGACACGGCCGTGTATTA | | | | | | | TSVTAADTAVYYCA | | | | | | |
| | | CTGTGCGAGAATACACGACTACGGTGGCCACATTAGTGCTT | | | | | | | RIHDYGGHISAFDM | | | | | | |
| | | TTGATATGTGGGGCCAAGGGACCACGGTCACCGTCTCCTCA | | | | | | | WGQGTTVTVSS | | | | | | |
| ADI-81595 | 1286 | TGGGTCCTCGGTGAAGGTCTCCTGCAAGGCTTCTGGAGGCA | | | | | | 6892 | QVQLQQSGAEVKKP | | 7245 | 22 | | 17 | |
| | | CCTTCAGCTCCTATGGTCTCACCTGGGTGCGACAGGCCCCT | | | | | | | GSSVKVSCKASGGT | | | | | | |
| | | GGACAAGGACTTGAGTTCATGGGAAGGATCCTCCCCCTCAT | | | | | | | FSSYGLTWVRQAPG | | | | | | |
| | | TGGTGTTCCTGACTACGCACAGAGCTTCCAGGGCAGAGTCA | | | | | | | QGLEFMGRILPLIG | | | | | | |
| | | CGATCACCGCGGACACATCCACGGGCACAGCCTACGTGGAG | | | | | | | VPDYAQSFQGRVTI | | | | | | |
| | | GTGAACAGCCTGAGATCTGAGGACACGGCCCTATATTATTG | | | | | | | TADTSTGTAYVEVN | | | | | | |
| | | TGCGAGAGAACGGGGATACAGTGGCTACGGCTCAGCGGTTT | | | | | | | SLRSEDTALYYCAR | | | | | | |
| | | ACTTTGACATCTGGGGCCAGGGCACCCTGGTCACCGTCTCC | | | | | | | ERGYSGYGSAVYFD | | | | | | |
| | | TCA | | | | | | | IWGQGTLVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81587 | 48 | IGHV4- | QVQLQ | 5363 | GSIGSN | 5565 | WIRQSA | 5798 | RIYASG | 5923 | RVTMSL | 6230 | ARGGGYD | 6535 | WGQGTT |
| | | 61 | ESGPG | | SFYLT | | GKGLEW | | GTSYNP | | DTSKNQ | | ELLVFGA | | VTVSS |
| | | | LVEPS | | | | LG | | SLQS | | MFLRLT | | FDI | | |
| | | | QTLSL | | | | | | | | SVTAAD | | | | |
| | | | TCTVS | | | | | | | | TAVYYC | | | | |
| | | | G | | | | | | | | | | | | |
| ADI-81597 | 57 | IGHV1- | QVQLV | 5364 | YTFTDY | 5566 | WLRQAP | 5799 | WLNPQT | 5924 | RVSMTG | 6231 | ARGGYCS | 6536 | WGQGSL |
| | | 2 | QSGAE | | YIH | | GQGFEW | | GDTNYA | | DTSIST | | AYSCFHG | | VTVSS |
| | | | VKKPG | | | | MG | | QKFQG | | AYMELR | | AWFAP | | |
| | | | ASAKV | | | | | | | | RLTSDD | | | | |
| | | | SCRAS | | | | | | | | TAFYYC | | | | |
| | | | G | | | | | | | | | | | | |
| ADI-81693 | 63 | IGHV3- | EVQLL | 5354 | IIVSWN | 5567 | WIRQAP | 5800 | VMYPGG | 5925 | RFTISR | 6232 | ARQENND | 6523 | WGQGTT |
| | | 53 | ETGGG | | YMT | | GKGLEW | | STFYAD | | DHSKNI | | AFDI | | VTVSS |
| | | | LIQPG | | | | VS | | SVRG | | LYLQMI | | | | |
| | | | GSLRL | | | | | | | | NLRVED | | | | |
| | | | SCAAS | | | | | | | | TAVYYC | | | | |
| | | | E | | | | | | | | | | | | |
| ADI-81783 | 59 | IGHV4- | EVQLL | 5365 | ASMNSR | 5568 | WVRQTP | 5801 | EISHSG | 571 | RLTILL | 6233 | ARAQGIF | 6537 | WGQGIL |
| | | 4 | ESGPG | | NWWA | | GQGLEW | | STNYNP | | DKSKSQ | | DY | | VTVSS |
| | | | LVKPS | | | | IG | | SLKS | | FSLRLS | | | | |
| | | | GTLSL | | | | | | | | SVTAAD | | | | |
| | | | TCAVS | | | | | | | | TAVYYC | | | | |
| | | | G | | | | | | | | | | | | |
| ADI-81823 | 4 | IGHV1- | EVQLV | 5366 | YTFSNH | 5569 | WVRQAP | 477 | IVNPDA | 5926 | RLTLTS | 6234 | ARDFSFI | 6538 | WGQGTL |
| | | 46 | ESGAE | | YIH | | GQGPEW | | GSTTYA | | DTSTST | | VATTAFE | | VTVSS |
| | | | VRKPG | | | | MG | | QKFQG | | VFMELS | | Y | | |
| | | | ASVKV | | | | | | | | SLRSDD | | | | |
| | | | SCRAS | | | | | | | | TAVYYC | | | | |
| | | | G | | | | | | | | | | | | |
| ADI-81888 | 64 | IGHV4- | EVQLL | 5365 | DSITSH | 5570 | WVRQPP | 444 | EVFHSG | 5927 | RVTISA | 6235 | ARVAGLE | 6539 | WGPGTL |
| | | 4 | ESGPG | | SWWS | | GKGLEW | | STEYSP | | DKSRDQ | | DN | | VTVSS |
| | | | LVKPS | | | | IG | | SLKS | | FSLKLK | | | | |
| | | | GTLSL | | | | | | | | SVTAAD | | | | |
| | | | TCAVS | | | | | | | | TAVYYC | | | | |
| | | | G | | | | | | | | | | | | |
| ADI-81593 | 53 | IGHV1- | QVQLV | 5367 | GTFSSY | 5517 | WVRQAP | 433 | GIIPIL | 5928 | RVTITA | 6236 | VRDRGYS | 6540 | WGRGTL |
| | | 69 | QSGAE | | GIS | | GQGLEW | | GTTSSA | | DKSTSM | | GYGSNYY | | VTVSS |
| | | | MRKPG | | | | MG | | HKFEG | | AYMELS | | FDL | | |
| | | | SSVKV | | | | | | | | SLRSED | | | | |
| | | | SCQAS | | | | | | | | TALYYC | | | | |
| | | | G | | | | | | | | | | | | |
| ADI-81884 | 60 | IGHV4- | EVQLL | 5335 | DSINSF | 5571 | WIRQPP | 445 | DIYHSG | 5929 | RVTISV | 6237 | ARGPKER | 1156 | WGQGIL |
| | | 59 | ESGPG | | YWA | | GKGLEW | | RTNYNP | | DTSKNN | | WENNWFD | | VTVSS |
| | | | LVKPS | | | | IG | | SLKS | | FSLKLS | | P | | |
| | | | ETLSL | | | | | | | | SLTTAD | | | | |
| | | | TCTVS | | | | | | | | TAVYFC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81587 | 1287 | TTCACAGACCCTGTCCCTCACCTGCACTGTCTCTGGTGGCT | | | | | | 6893 | QVQLQESGPGLVEP | | 7246 | 21 | | 18 | |
| | | CCATCGGCAGTAATAGTTTCTACTTGACCTGGATCCGGCAG | | | | | | | SQTLSLTCTVSGGS | | | | | | |
| | | TCCGCCGGGAAGGGACTGGAGTGGCTTGGGCGTATCTATGC | | | | | | | IGSNSFYLTWIRQS | | | | | | |
| | | CAGTGGGGGCACCAGCTACAACCCCTCCCTCCAGAGTCGAG | | | | | | | AGKGLEWLGRIYAS | | | | | | |
| | | TCACCATGTCACTAGACACGTCCAAGAACCAGATGTTCCTG | | | | | | | GGTSYNPSLQSRVT | | | | | | |
| | | AGGCTGACGTCTGTGACCGCCGCTGACACGGCCGTCTATTA | | | | | | | MSLDTSKNQMFLRL | | | | | | |
| | | TTGTGCGAGAGGGGGTGGCTACGATGAATTACTAGTTTTCG | | | | | | | TSVTAADTAVYYCA | | | | | | |
| | | GTGCTTTTGATATCTGGGGCCAAGGGACCACGGTCACCGTC | | | | | | | RGGGYDELLVFGAF | | | | | | |
| | | TCCTCA | | | | | | | DIWGQGTTVTVSS | | | | | | |
| ADI-81597 | 1289 | TGGGGCCTCAGCGAAGGTCTCCTGTAGGGCTTCTGGATACA | | | | | | 6894 | QVQLVQSGAEVKKP | | 7247 | 19 | | 15 | |
| | | CGTTCACCGACTACTATATTCACTGGCTACGACAGGCCCCT | | | | | | | GASAKVSCRASGYT | | | | | | |
| | | GGACAAGGGTTTGAGTGGATGGGATGGCTGAACCCTCAGAC | | | | | | | FTDYYIHWLRQAPG | | | | | | |
| | | TGGTGACACAAACTATGCGCAGAAGTTTCAGGGCAGGGTCT | | | | | | | QGFEWMGWLNPQTG | | | | | | |
| | | CCATGACCGGGGACACGTCCATCAGCACAGCCTACATGGAG | | | | | | | DTNYAQKFQGRVSM | | | | | | |
| | | CTGAGGAGGCTGACATCTGATGACACGGCCTTTTATTACTG | | | | | | | TGDTSISTAYMELR | | | | | | |
| | | TGCGAGAGGGGGATATTGTAGTGCTTACAGCTGCTTCCACG | | | | | | | RLTSDDTAFYYCAR | | | | | | |
| | | GGGCTTGGTTCGCCCCCTGGGGCCAGGGAAGCCTGGTCACC | | | | | | | GGYCSAYSCFHGAW | | | | | | |
| | | GTCTCCTCA | | | | | | | FAPWGQGSLVTVSS | | | | | | |
| ADI-81693 | 1287 | GAGGTGCAGCTGTTGGAGACTGGAGGAGGTTTGATCCAGCC | | | | | | 6895 | EVQLLETGGGLIQP | | 7248 | 18 | | 15 | |
| | | GGGGGGGTCCCTGAGACTCTCCTGTGCAGCCTCTGAGATCA | | | | | | | GGSLRLSCAASEII | | | | | | |
| | | TCGTCAGTTGGAACTACATGACCTGGATCCGCCAGGCTCCA | | | | | | | VSWNYMTWIRQAPG | | | | | | |
| | | GGGAAAGGTCTGGAGTGGGTCTCAGTTATGTATCCCGGTGG | | | | | | | KGLEWVSVMYPGGS | | | | | | |
| | | GAGCACTTTCTACGCAGACTCCGTGAGGGGCCGATTCACCA | | | | | | | TFYADSVRGRFTIS | | | | | | |
| | | TCTCTAGAGACCATTCCAAGAATATTCTGTATCTTCAAATG | | | | | | | RDHSKNILYLQMIN | | | | | | |
| | | ATCAACCTGAGAGTCGAGGACACGGCCGTGTACTACTGTGC | | | | | | | LRVEDTAVYYCARQ | | | | | | |
| | | GAGACAAGAGAATAATGATGCTTTTGATATTTGGGGACAGG | | | | | | | ENNDAFDIWGQGTT | | | | | | |
| | | GGACCACGGTCACCGTCTCCTCA | | | | | | | VTVSS | | | | | | |
| ADI-81783 | 6809 | GAGGTGCAGCTGTTGGAGTCGGGCCCAGGACTGGTGAAGCC | | | | | | 6896 | EVQLLESGPGLVKP | | 7249 | 18 | | 13 | |
| | | TTCGGGGACCCTGTCCCTCACCTGCGCTGTCTCTGGTGCCT | | | | | | | SGTLSLTCAVSGAS | | | | | | |
| | | CCATGAACAGTCGGAATTGGTGGGCTTGGGTCCGCCAGACC | | | | | | | MNSRNWWAWVRQTP | | | | | | |
| | | CCAGGGCAGGGGCTGGAATGGATTGGCGAAATCTCTCATAG | | | | | | | GQGLEWIGEISHSG | | | | | | |
| | | TGGGAGCACCAACTACAACCCGTCCCTCAAGAGTCGACTGA | | | | | | | STNYNPSLKSRLTI | | | | | | |
| | | CCATACTACTGGACAAGTCCAAGAGCCAGTTCTCCCTGAGG | | | | | | | LLDKSKSQFSLRLS | | | | | | |
| | | TTGAGCTCCGTGACCGCCGCGGACACGGCCGTCTATTACTG | | | | | | | SVTAADTAVYYCAR | | | | | | |
| | | TGCGAGAGCCCAAGGAATCTTTGACTACTGGGGCCAGGGAA | | | | | | | AQGIFDYWGQGILV | | | | | | |
| | | TTCTGGTCACCGTCTCCTCA | | | | | | | TVSS | | | | | | |
| ADI-81823 | 1286 | GAGGTGCAGCTGGTGGAGTCTGGGGCTGAGGTGAGGAAGCC | | | | | | 6897 | EVQLVESGAEVRKP | | 7250 | 17 | | 16 | |
| | | TGGGGCCTCAGTGAAGGTTTCCTGCAGGGCATCTGGATACA | | | | | | | GASVKVSCRASGYT | | | | | | |
| | | CCTTCAGCAACCACTATATACACTGGGTGCGGCAGGCCCCT | | | | | | | FSNHYIHWVRQAPG | | | | | | |
| | | GGACAAGGCCCTGAGTGGATGGGAATAGTCAACCCTGATGC | | | | | | | QGPEWMGIVNPDAG | | | | | | |
| | | TGGTAGCACAACCTACGCACAGAAATTCCAGGGCAGACTCA | | | | | | | STTYAQKFQGRLTL | | | | | | |
| | | CCCTGACCAGCGACACGTCCACGAGCACAGTGTTCATGGAG | | | | | | | TSDTSTSTVFMELS | | | | | | |
| | | TTGAGCAGCCTGAGATCTGACGACACGGCCGTATATTATTG | | | | | | | SLRSDDTAVYYCAR | | | | | | |
| | | TGCGAGAGATTTCTCATTTATAGTGGCAACGACAGCCTTTG | | | | | | | DFSFIVATTAFEYW | | | | | | |
| | | AGTATTGGGGCCAGGGAACCCTGGTCACCGTCTCCTCA | | | | | | | GQGTLVTVSS | | | | | | |
| ADI-81888 | 1302 | GAGGTGCAGCTGTTGGAGTCGGGCCCAGGACTGGTGAAGCC | | | | | | 6898 | EVQLLESGPGLVKP | | 7251 | 17 | | 12 | |
| | | TTCGGGGACCCTGTCCCTCACATGCGCTGTGTCTGGTGACT | | | | | | | SGTLSLTCAVSGDS | | | | | | |
| | | CCATCACCAGTCATAGTTGGTGGAGTTGGGTCCGCCAGCCC | | | | | | | ITSHSWWSWVRQPP | | | | | | |
| | | CCAGGGAAGGGGCTGGAGTGGATTGGGGAAGTCTTTCATAG | | | | | | | GKGLEWIGEVFHSG | | | | | | |
| | | TGGGAGTACCGAGTACAGTCCGTCCCTCAAGAGTCGAGTCA | | | | | | | STEYSPSLKSRVTI | | | | | | |
| | | CCATATCAGCAGACAAGTCCAGGGACCAGTTCTCACTGAAG | | | | | | | SADKSRDQFSLKLK | | | | | | |
| | | TTGAAGTCTGTGACCGCCGCAGACACGGCCGTATATTACTG | | | | | | | SVTAADTAVYYCAR | | | | | | |
| | | TGCGAGAGTGGCTGGTCTTTTTGACAACTGGGGCCCGGGAA | | | | | | | VAGLFDNWGPGTLV | | | | | | |
| | | CCCTGGTCACCGTCTCCTCA | | | | | | | TVSS | | | | | | |
| ADI-81593 | 1288 | TGGGTCCTCGGTGAAGGTCTCCTGCCAGGCTTCTGGAGGCA | | | | | | 6899 | QVQLVQSGAEMRKP | | 7252 | 15 | | 12 | |
| | | CCTTCAGCAGTTATGGAATCAGCTGGGTGCGACAGGCCCCT | | | | | | | GSSVKVSCQASGGT | | | | | | |
| | | GGACAAGGGCTTGAGTGGATGGGAGGGATCATCCCTATCCT | | | | | | | FSSYGISWVRQAPG | | | | | | |
| | | TGGTACGACAAGTTCCGCACACAAATTCGAGGGCAGAGTCA | | | | | | | QGLEWMGGIIPILG | | | | | | |
| | | CGATTACCGCGGACAAATCCACTAGTATGGCCTACATGGAG | | | | | | | TTSSAHKFEGRVTI | | | | | | |
| | | CTGAGCAGCCTGAGGTCTGAGGACACGGCCCTGTACTACTG | | | | | | | TADKSTSMAYMELS | | | | | | |
| | | TGTGAGAGATCGTGGATACAGTGGCTACGGATCAAACTACT | | | | | | | SLRSEDTALYYCVR | | | | | | |
| | | ACTTCGATCTCTGGGGCCGTGGCACCCTGGTCACCGTCTCC | | | | | | | DRGYSGYGSNYYFD | | | | | | |
| | | TCA | | | | | | | LWGRGTLVTVSS | | | | | | |
| ADI-81884 | 6809 | GAGGTGCAGCTGTTGGAGTCGGGCCCAGGGCTGGTGAAGCC | | | | | | 6900 | EVQLLESGPGLVKP | | 7253 | 15 | | 11 | |
| | | TTCGGAGACCCTGTCCCTCACCTGCACTGTCTCTGGTGACT | | | | | | | SETLSLTCTVSGDS | | | | | | |
| | | CCATCAATAGTTTCTATTGGGCCTGGATCCGGCAGCCCCCA | | | | | | | INSFYWAWIRQPPG | | | | | | |
| | | GGGAAGGGACTGGAGTGGATTGGGGACATCTATCACAGTGG | | | | | | | KGLEWIGDIYHSGR | | | | | | |
| | | GAGGACCAATTACAATCCCTCCCTCAAGAGTCGAGTCACCA | | | | | | | TNYNPSLKSRVTIS | | | | | | |
| | | TATCAGTGGACACGTCCAAAAATAACTTCTCCCTGAAGTTG | | | | | | | VDTSKNNFSLKLSS | | | | | | |
| | | AGTTCTCTGACCACTGCGGACACGGCCGTCTATTTTTGTGC | | | | | | | LTTADTAVYFCARG | | | | | | |
| | | GAGAGGCCCTAAAGAGCGGTGGTTCAACAACTGGTTCGACC | | | | | | | PKERWFNNWFDPWG | | | | | | |
| | | CCTGGGGCCAAGGAATTCTGGTCACCGTCTCCTCA | | | | | | | QGILVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81545 | 12 | IGHV3- | QVQLV | 12 | FTFSTS | 5555 | WVRQGR | 5802 | AIGTSG | 5930 | RFSVSR | 6238 | ARGEALG | 6541 | WGQGTT |
| | | 13 | QSGGG | | DMH | | GRGLEW | | DTYYAD | | EDGKNS | | VEPYYYY | | VTVSS |
| | | | LVQPG | | | | VS | | SVKG | | FYLQMN | | FMDI | | |
| | | | GSLRL | | | | | | | | SLRAGD | | | | |
| | | | SCAAS | | | | | | | | TAVYYC | | | | |
| | | | G | | | | | | | | | | | | |
| ADI-81555 | 21 | IGHV3- | QVQLQ | 20 | FTFENY | 5572 | WVRQSP | 5803 | TISSDA | 5931 | RFTISR | 6239 | AKDTLVD | 6542 | WGQGTL |
| | | 30 | QSGGG | | DMH | | GKGLEW | | SIKYYG | | DNSKNT | | IAVVTLI | | VTVSS |
| | | | VVQPG | | | | VA | | DSVKG | | LYLQMS | | DY | | |
| | | | RSLRL | | | | | | | | SLRAED | | | | |
| | | | SCAAS | | | | | | | | TAVYFC | | | | |
| | | | G | | | | | | | | | | | | |
| ADI-81838 | 17 | IGHV2- | QVQLM | 5368 | FSLNTA | 5573 | WIRQPP | 441 | LVYWDG | 5932 | RVTITK | 6240 | AHHSIPT | 6543 | WGQGIL |
| | | 5 | ESGPT | | GVGLA | | GKALEW | | DRRYSP | | DTSKNQ | | IFTS | | VTVSS |
| | | | LVKPT | | | | LA | | FLKT | | VVLTMT | | | | |
| | | | QTLTL | | | | | | | | NMDSVD | | | | |
| | | | TCSFS | | | | | | | | TATYFC | | | | |
| | | | G | | | | | | | | | | | | |
| ADI-81623 | 54 | IGHV3- | QVQLV | 5369 | FTFRIY | 5574 | WVRQAP | 431 | VISHDG | 5933 | RFTISR | 6241 | ARATTDY | 6544 | WGQGTL |
| | | 30 | QSGGG | | AFH | | GKGLEW | | SETYYA | | DNSKNT | | CGGDCYP | | VTVSS |
| | | | VVQPG | | | | VA | | DSVKG | | LSLQMH | | IDS | | |
| | | | RSLRL | | | | | | | | SLRADD | | | | |
| | | | SCPVS | | | | | | | | TAVYYC | | | | |
| | | | G | | | | | | | | | | | | |
| ADI-81551 | 18 | IGHV1- | QVQLV | 5370 | FTFITS | 5575 | WVRQTR | 5804 | WIAVGS | 5934 | RLTITT | 6242 | ASPHCRG | 6545 | WGQGTT |
| | | 58 | QSGPE | | AVQ | | GQRLEW | | GNTEYA | | DMSTST | | GSCLDGF | | VTVSS |
| | | | VKKPG | | | | MG | | QKFQE | | TYMELS | | DI | | |
| | | | TSVKV | | | | | | | | SLRSED | | | | |
| | | | SCKTS | | | | | | | | TAVYYC | | | | |
| | | | G | | | | | | | | | | | | |
| ADI-81594 | 54 | IGHV3- | QVQLV | 5371 | FTFSRF | 5576 | WVRQAP | 472 | LISFDG | 5935 | RFTISR | 789 | ARATTDY | 6546 | WGRGIP |
| | | 30 | QSGGG | | SMH | | GKGLEW | | SDKYYT | | DNSKNT | | CGGDCYP | | VTVSS |
| | | | VVQPG | | | | VT | | DSVKG | | LYLQMN | | IDN | | |
| | | | RSLRL | | | | | | | | SLRTED | | | | |
| | | | SCLAS | | | | | | | | TAVYYC | | | | |
| | | | G | | | | | | | | | | | | |
| ADI-81681 | 51 | IGHV3- | RSTLK | 5372 | FTFEDY | 208 | WVRQAP | 437 | GVSWNS | 672 | RFIVSR | 6243 | AKLGALS | 6547 | WGKGTT |
| | | 9 | ESGGG | | AMH | | GKGLEW | | GIIGYA | | DNAKNS | | RQYDFRT | | VTVSS |
| | | | LVQPG | | | | VS | | DSVKG | | LYLYMS | | GENHQYY | | |
| | | | RSLRL | | | | | | | | SLRVED | | MDV | | |
| | | | SCAAS | | | | | | | | TAFYYC | | | | |
| | | | G | | | | | | | | | | | | |
| ADI-81833 | 12 | IGHV3- | EVQLL | 5373 | FSFSDY | 5577 | WVRQAP | 431 | VIWYDG | 5936 | RFTISR | 6244 | ARLATIP | 6548 | WGKGTT |
| | | 33 | ESGGG | | GMH | | GKGLEW | | SNRYYL | | DNSKNT | | WSYYMDV | | VTVSS |
| | | | VVQPG | | | | VA | | DSVQG | | VYLQMN | | | | |
| | | | RSLRL | | | | | | | | NLRAED | | | | |
| | | | SCAAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81545 | 1287 | TGGGGGGTCCCTGAGACTCTCCTGTGCAGCCTCTGGATTCA | | | | | | 6901 | QVQLVQSGGGLVQP | | 7254 | 14 | | 13 | |
| | | CCTTCAGTACCTCCGACATGCACTGGGTCCGTCAAGGTAGA | | | | | | | GGSLRLSCAASGFT | | | | | | |
| | | GGAAGAGGTCTGGAATGGGTCTCAGCTATTGGTACTTCTGG | | | | | | | FSTSDMHWVRQGRG | | | | | | |
| | | TGACACATACTATGCAGACTCCGTGAAGGGCCGATTTTCGG | | | | | | | RGLEWVSAIGTSGD | | | | | | |
| | | TCTCCAGAGAAGATGGCAAAAACTCCTTCTATCTTCAAATG | | | | | | | TYYADSVKGRFSVS | | | | | | |
| | | AACAGCCTGAGAGCCGGAGACACGGCTGTCTATTACTGTGC | | | | | | | REDGKNSFYLQMNS | | | | | | |
| | | AAGAGGAGAGGCCCTTGGAGTGGAGCCCTATTATTACTATT | | | | | | | LRAGDTAVYYCARG | | | | | | |
| | | TTATGGACATTTGGGGCCAAGGGACCACGGTCACCGTCTCC | | | | | | | EALGVEPYYYYFMD | | | | | | |
| | | TCA | | | | | | | IWGQGTTVTVSS | | | | | | |
| ADI-81555 | 1286 | CAGGTGCAGCTGCAGCAGTCTGGGGGGGGCGTGGTCCAGCC | | | | | | 6902 | QVQLQQSGGGVVQP | | 7255 | 14 | | 11 | |
| | | TGGGAGGTCCCTGAGACTCTCCTGTGCAGCCTCTGGATTCA | | | | | | | GRSLRLSCAASGFT | | | | | | |
| | | CCTTCGAAAACTATGACATGCACTGGGTCCGCCAGTCTCCA | | | | | | | FENYDMHWVRQSPG | | | | | | |
| | | GGCAAGGGGCTGGAGTGGGTGGCAACTATATCATCTGATGC | | | | | | | KGLEWVATISSDAS | | | | | | |
| | | AAGTATTAAATATTATGGAGACTCCGTGAAGGGCCGATTCA | | | | | | | IKYYGDSVKGRFTI | | | | | | |
| | | CCATCTCCAGAGACAATTCCAAGAACACGCTGTATCTGCAA | | | | | | | SRDNSKNTLYLQMS | | | | | | |
| | | ATGAGCAGCCTGAGAGCTGAGGACACGGCTGTGTATTTCTG | | | | | | | SLRAEDTAVYFCAK | | | | | | |
| | | TGCGAAAGATACCCTCGTGGATATAGCTGTGGTAACCCTGA | | | | | | | DTLVDIAVVTLIDY | | | | | | |
| | | TTGACTACTGGGGCCAGGGAACCCTGGTCACCGTCTCCTCA | | | | | | | WGQGTLVTVSS | | | | | | |
| ADI-81838 | 6809 | CAGGTCCAGCTGATGGAGTCTGGTCCTACGCTGGTGAAACC | | | | | | 6903 | QVQLMESGPTLVKP | | 7256 | 14 | | 13 | |
| | | CACACAGACCCTCACGCTGACCTGCTCCTTCTCTGGGTTCT | | | | | | | TQTLTLTCSFSGFS | | | | | | |
| | | CACTCAACACTGCTGGAGTGGGTTTGGCCTGGATACGTCAG | | | | | | | LNTAGVGLAWIRQP | | | | | | |
| | | CCCCCAGGAAAGGCCCTGGAGTGGCTTGCCCTCGTTTATTG | | | | | | | PGKALEWLALVYWD | | | | | | |
| | | GGATGGTGATAGGCGCTATAGTCCATTTTTGAAGACCAGGG | | | | | | | GDRRYSPFLKTRVT | | | | | | |
| | | TCACCATCACCAAGGACACCTCCAAAAACCAGGTGGTCCTT | | | | | | | ITKDTSKNQVVLTM | | | | | | |
| | | ACAATGACCAACATGGACTCTGTGGACACAGCCACATATTT | | | | | | | TNMDSVDTATYFCA | | | | | | |
| | | CTGTGCACACCATTCAATTCCTACAATTTTTACCTCCTGGG | | | | | | | HHSIPTIFTSWGQG | | | | | | |
| | | GCCAGGGAATCCTGGTCACCGTCTCCTCA | | | | | | | ILVTVSS | | | | | | |
| ADI-81623 | 1286 | TGGGAGGTCCCTGAGACTCTCCTGTCCAGTCTCTGGATTCA | | | | | | 6904 | QVQLVQSGGGVVQP | | 7257 | 13 | | 11 | |
| | | CCTTCAGGATCTACGCTTTCCACTGGGTCCGCCAGGCTCCA | | | | | | | GRSLRLSCPVSGFT | | | | | | |
| | | GGCAAGGGGCTGGAGTGGGTGGCAGTTATATCGCATGATGG | | | | | | | FRIYAFHWVRQAPG | | | | | | |
| | | AAGCGAGACATACTACGCAGACTCCGTGAAGGGCCGATTCA | | | | | | | KGLEWVAVISHDGS | | | | | | |
| | | CCATCTCCAGAGACAATTCCAAGAACACGTTGTCTCTGCAA | | | | | | | ETYYADSVKGRFTI | | | | | | |
| | | ATGCACAGCCTGAGAGCTGACGACACGGCTGTCTATTACTG | | | | | | | SRDNSKNTLSLQMH | | | | | | |
| | | TGCAAGAGCGACTACCGATTATTGTGGCGGTGACTGCTATC | | | | | | | SLRADDTAVYYCAR | | | | | | |
| | | CCATTGATTCCTGGGGCCAGGGAACCCTGGTCACCGTCTCC | | | | | | | ATTDYCGGDCYPID | | | | | | |
| | | TCA | | | | | | | SWGQGTLVTVSS | | | | | | |
| ADI-81551 | 1287 | CAGGTCCAGCTGGTGCAGTCTGGGCCTGAGGTGAAGAAGCC | | | | | | 6905 | QVQLVQSGPEVKKP | | 7258 | 11 | | 10 | |
| | | TGGGACCTCAGTGAAGGTCTCCTGCAAGACTTCTGGATTCA | | | | | | | GTSVKVSCKTSGFT | | | | | | |
| | | CCTTTATTACCTCTGCTGTGCAGTGGGTGCGACAGACTCGT | | | | | | | FITSAVQWVRQTRG | | | | | | |
| | | GGACAACGCCTTGAGTGGATGGGGTGGATCGCCGTTGGCAG | | | | | | | QRLEWMGWIAVGSG | | | | | | |
| | | TGGGAACACAGAGTACGCACAGAAGTTCCAGGAAAGACTCA | | | | | | | NTEYAQKFQERLTI | | | | | | |
| | | CTATTACCACGGACATGTCCACAAGCACAACCTACATGGAG | | | | | | | TTDMSTSTTYMELS | | | | | | |
| | | CTGAGCAGCCTGAGATCCGAGGACACGGCCGTATATTACTG | | | | | | | SLRSEDTAVYYCAS | | | | | | |
| | | TGCGTCACCACACTGTCGTGGTGGTAGCTGCCTGGATGGTT | | | | | | | PHCRGGSCLDGFDI | | | | | | |
| | | TTGATATCTGGGGCCAAGGGACCACGGTCACCGTCTCCTCA | | | | | | | WGQGTTVTVSS | | | | | | |
| ADI-81594 | 6810 | TGGGAGGTCCCTGAGACTCTCCTGTCTAGCCTCTGGATTCA | | | | | | 6906 | QVQLVQSGGGVVQP | | 7259 | 11 | | 9 | |
| | | CCTTCAGTCGATTTTCTATGCACTGGGTCCGCCAGGCTCCA | | | | | | | GRSLRLSCLASGFT | | | | | | |
| | | GGCAAGGGGCTGGAGTGGGTGACACTTATATCATTTGATGG | | | | | | | FSRFSMHWVRQAPG | | | | | | |
| | | AAGCGATAAATACTACACAGACTCCGTGAAGGGCCGATTCA | | | | | | | KGLEWVTLISFDGS | | | | | | |
| | | CCATCTCCAGAGACAATTCCAAGAACACGCTGTATCTGCAG | | | | | | | DKYYTDSVKGRFTI | | | | | | |
| | | ATGAACAGTCTGAGAACTGAGGACACGGCTGTGTATTACTG | | | | | | | SRDNSKNTLYLQMN | | | | | | |
| | | TGCGAGAGCGACTACCGATTACTGTGGTGGCGACTGCTATC | | | | | | | SLRTEDTAVYYCAR | | | | | | |
| | | CCATTGACAACTGGGGCCGGGGAATCCCGGTCACCGTCTCC | | | | | | | ATTDYCGGDCYPID | | | | | | |
| | | TCA | | | | | | | NWGRGIPVTVSS | | | | | | |
| ADI-81681 | 1291 | AGGTCCACGCTGAAGGAGTCTGGGGGAGGCCTGGTACAGCC | | | | | | 6907 | RSTLKESGGGLVQP | | 7260 | 11 | | 9 | |
| | | TGGCAGGTCCCTGAGACTCTCCTGTGCAGCCTCTGGATTCA | | | | | | | GRSLRLSCAASGFT | | | | | | |
| | | CGTTTGAAGATTATGCCATGCACTGGGTCCGGCAAGCTCCA | | | | | | | FEDYAMHWVRQAPG | | | | | | |
| | | GGGAAGGGCCTGGAGTGGGTCTCAGGTGTTAGTTGGAATAG | | | | | | | KGLEWVSGVSWNSG | | | | | | |
| | | CGGTATCATAGGCTATGCGGACTCTGTGAAGGGCCGATTCA | | | | | | | IIGYADSVKGRFIV | | | | | | |
| | | TCGTCTCCAGAGACAACGCCAAGAACTCCCTATATCTCTAC | | | | | | | SRDNAKNSLYLYMS | | | | | | |
| | | ATGAGCAGCCTGAGAGTCGAGGACACGGCCTTCTATTACTG | | | | | | | SLRVEDTAFYYCAK | | | | | | |
| | | TGCAAAATTGGGGGCTTTGTCTCGCCAATACGATTTTCGGA | | | | | | | LGALSRQYDFRTGE | | | | | | |
| | | CGGGGGAAAATCACCAGTATTACATGGACGTCTGGGGCAAG | | | | | | | NHQYYMDVWGKGTT | | | | | | |
| | | GGGACCACGGTCACCGTCTCCTCA | | | | | | | VTVSS | | | | | | |
| ADI-81833 | 1291 | GAGGTGCAGCTGTTGGAGTCGGGGGGAGGCGTGGTCCAGCC | | | | | | 6908 | EVQLLESGGGVVQP | | 7261 | 11 | | 7 | |
| | | TGGGAGGTCCCTGAGACTCTCCTGTGCAGCGTCTGGATTCA | | | | | | | GRSLRLSCAASGFS | | | | | | |
| | | GTTTCAGTGACTATGGCATGCACTGGGTCCGCCAGGCTCCA | | | | | | | FSDYGMHWVRQAPG | | | | | | |
| | | GGCAAGGGGCTGGAGTGGGTGGCAGTTATATGGTATGATGG | | | | | | | KGLEWVAVIWYDGS | | | | | | |
| | | AAGTAACAGGTATTATTTAGACTCTGTGCAGGGCCGATTCA | | | | | | | NRYYLDSVQGRFTI | | | | | | |
| | | CCATCTCCAGAGACAATTCCAAGAATACGGTGTATCTCCAA | | | | | | | SRDNSKNTVYLQMN | | | | | | |
| | | ATGAACAACTTGAGAGCCGAGGACACGGCTGTCTATTACTG | | | | | | | NLRAEDTAVYYCAR | | | | | | |
| | | TGCGAGATTGGCTACGATTCCCTGGTCCTACTATATGGACG | | | | | | | LATIPWSYYMDVWG | | | | | | |
| | | TCTGGGGCAAAGGGACCACGGTCACCGTCTCCTCA | | | | | | | KGTTVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81672 | 43 | IGHV1- | QVQLV | 35 | YTFTSY | 367 | WVRQAP | 433 | WINTFN | 5937 | RVTMTT | 6245 | ARVGTDY | 6549 | WGQGTL |
| | | 18 | QSGAE | | GIS | | GQGLEW | | GDTNYP | | DTSTTT | | VWGSSIY | | VTVSS |
| | | | VKKPG | | | | MG | | QKFQG | | VHMELR | | FFDY | | |
| | | | ASVKV | | | | | | | | SLRSDD | | | | |
| | | | SCKAS | | | | | | | | TALYYC | | | | |
| | | | G | | | | | | | | | | | | |
| ADI-81872 | 49 | IGHV4- | EVQLV | 5347 | GSISSR | 5578 | WVRQSP | 438 | EIFHTG | 5938 | RVTMSV | 6246 | ARSAGLL | 6550 | WGQGIL |
| | | 4 | ESGPG | | NWWS | | GKGLEW | | STAYNP | | DKSKNQ | | DY | | VTVSS |
| | | | LVKPS | | | | IG | | SLKS | | FSLRLS | | | | |
| | | | GTLSL | | | | | | | | SVNAAD | | | | |
| | | | TCAVS | | | | | | | | TAVYYC | | | | |
| | | | G | | | | | | | | | | | | |
| ADI-81679 | 49 | IGHV3- | EVQLL | 5355 | FTFSNS | 5579 | WVRQTT | 5805 | TIGTAG | 618 | RFTISR | 6247 | ARGEQRM | 6551 | WGKGTT |
| | | 13 | ESGGG | | DMH | | GNSLEW | | DTYYPD | | ENAKNS | | MISGVVP | | VTVSS |
| | | | LVQPG | | | | VS | | SVKG | | LSLQMN | | RYYYYMD | | |
| | | | GSLRL | | | | | | | | SLRAGD | | V | | |
| | | | SCAAS | | | | | | | | SAVYYC | | | | |
| | | | G | | | | | | | | | | | | |
| ADI-81553 | 19 | IGHV1- | QVQLV | 35 | YRFTSY | 5580 | WVRQAP | 433 | WISVYN | 5939 | RVTMTT | 6248 | ARDTIYM | 6552 | WGQGTT |
| | | 18 | QSGAE | | GIS | | GQGLEW | | GDTNYA | | DTSTNT | | VRGVLDA | | VTVSS |
| | | | VKKPG | | | | MG | | QKVQG | | AYMQLR | | FDI | | |
| | | | ASVKV | | | | | | | | NLRSDD | | | | |
| | | | SCKAS | | | | | | | | TAVYYC | | | | |
| | | | G | | | | | | | | | | | | |
| ADI-81636 | 9 | IGHV1- | EVQLV | 5358 | GTFSSY | 385 | WVRQAP | 433 | RIFPIL | 5940 | RVTISA | 6249 | ARDRAAA | 6553 | WGQGTL |
| | | 69 | QSGAE | | TFS | | GQGLEW | | GIPNYA | | DRSTST | | DTNTLPT | | VTVSS |
| | | | VKKPG | | | | MG | | QKFQD | | AYMELS | | YYFDY | | |
| | | | SSVKV | | | | | | | | SLRSED | | | | |
| | | | SCKAS | | | | | | | | TAVYFC | | | | |
| | | | G | | | | | | | | | | | | |
| ADI-81536 | 5 | IGHV2- | QVTLK | 5374 | FSFSTS | 5581 | WIRQPP | 441 | LIYWDG | 5941 | RLTITR | 6250 | AHHDIPT | 6554 | WGTGTT |
| | | 5 | ESGPA | | GVGVA | | GKALEW | | DKRYSP | | DTSKNQ | | IFSV | | VTVSS |
| | | | LVKPK | | | | LA | | SLKS | | VVLTMT | | | | |
| | | | QTLTL | | | | | | | | NMDPVD | | | | |
| | | | TCTFS | | | | | | | | TATYYC | | | | |
| | | | G | | | | | | | | | | | | |
| ADI-81684 | 54 | IGHV3- | EVQLV | 5332 | FTFSTY | 244 | WVRQVT | 468 | AIGTGG | 5942 | RFTISR | 6251 | ARGLHSV | 6555 | WGKGTT |
| | | 13 | ESGGG | | DMH | | GKGLEW | | HTYYPD | | ENAKSS | | AAGGTIR | | VTVSS |
| | | | LVQPG | | | | VS | | SVKG | | LYLQMN | | YYYYMDV | | |
| | | | GSLRL | | | | | | | | SLRAGD | | | | |
| | | | SCAAS | | | | | | | | TAVYYC | | | | |
| | | | G | | | | | | | | | | | | |
| ADI-81794 | 70 | IGHV5- | EVQLL | 5375 | YSFISN | 5582 | WVRQMP | 476 | IIYPGD | 635 | QVTMSV | 6252 | ARLEVGG | 6556 | WGQGTL |
| | | 51 | ESGAQ | | WIG | | GKGLEW | | SDTRYS | | DKSIST | | RDY | | VTVSS |
| | | | VKKPG | | | | MG | | PSFQG | | AYLQWT | | | | |
| | | | ESLKI | | | | | | | | SLKASD | | | | |
| | | | SCKGS | | | | | | | | TAMYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81672 | 1286 | TGGGGCCTCAGTGAAGGTCTCCTGCAAGGCCTCTGGTTACA | | | | | | 6909 | QVQLVQSGAEVKKP | | 7262 | 10 | | 10 | |
| | | CCTTTACCAGCTACGGAATCAGCTGGGTGCGACAGGCCCCT | | | | | | | GASVKVSCKASGYT | | | | | | |
| | | GGACAAGGGCTTGAGTGGATGGGATGGATCAACACTTTCAA | | | | | | | FTSYGISWVRQAPG | | | | | | |
| | | TGGTGACACAAACTATCCACAGAAGTTCCAGGGCAGAGTCA | | | | | | | QGLEWMGWINTFNG | | | | | | |
| | | CCATGACCACAGACACATCCACGACCACAGTCCACATGGAG | | | | | | | DTNYPQKFQGRVTM | | | | | | |
| | | CTGAGGAGCCTGAGATCTGACGACACGGCCCTGTATTACTG | | | | | | | TTDTSTTTVHMELR | | | | | | |
| | | TGCGAGAGTAGGAACTGATTACGTTTGGGGGAGTTCCATCT | | | | | | | SLRSDDTALYYCAR | | | | | | |
| | | ACTTCTTTGACTACTGGGGCCAGGGAACCCTGGTCACCGTC | | | | | | | VGTDYVWGSSIYFF | | | | | | |
| | | TCCTCA | | | | | | | DYWGQGTLVTVSS | | | | | | |
| ADI-81872 | 6809 | GAGGTGCAGCTGGTGGAGTCGGGCCCAGGACTGGTGAAGCC | | | | | | 6910 | EVQLVESGPGLVKP | | 7263 | 10 | | 8 | |
| | | TTCGGGGACCCTGTCCCTCACTTGCGCTGTCTCTGGCGGCT | | | | | | | SGTLSLTCAVSGGS | | | | | | |
| | | CCATCAGCAGTAGAAATTGGTGGAGTTGGGTCCGCCAGTCC | | | | | | | ISSRNWWSWVRQSP | | | | | | |
| | | CCAGGGAAGGGGCTGGAGTGGATTGGAGAAATCTTTCATAC | | | | | | | GKGLEWIGEIFHTG | | | | | | |
| | | TGGGAGTACCGCCTACAACCCGTCCCTCAAGAGTCGAGTCA | | | | | | | STAYNPSLKSRVTM | | | | | | |
| | | CCATGTCGGTGGACAAGTCCAAGAACCAGTTCTCCCTGAGA | | | | | | | SVDKSKNQFSLRLS | | | | | | |
| | | CTGAGCTCTGTGAACGCCGCGGACACGGCCGTGTATTACTG | | | | | | | SVNAADTAVYYCAR | | | | | | |
| | | TGCGAGGTCGGCTGGCCTCCTTGACTACTGGGGCCAGGGAA | | | | | | | SAGLLDYWGQGILV | | | | | | |
| | | TCCTGGTCACCGTCTCCTCA | | | | | | | TVSS | | | | | | |
| ADI-81679 | 1291 | GAGGTGCAGCTGTTGGAGTCTGGGGGAGGCTTGGTACAGCC | | | | | | 6911 | EVQLLESGGGLVQP | | 7264 | 9 | | 9 | |
| | | TGGGGGGTCCCTGAGACTCTCCTGTGCGGCCTCTGGATTCA | | | | | | | GGSLRLSCAASGFT | | | | | | |
| | | CCTTCAGTAACTCCGACATGCACTGGGTCCGCCAAACTACA | | | | | | | FSNSDMHWVRQTTG | | | | | | |
| | | GGAAATAGTCTGGAGTGGGTCTCAACTATTGGTACTGCTGG | | | | | | | NSLEWVSTIGTAGD | | | | | | |
| | | GGATACATACTATCCAGACTCCGTGAAGGGCCGATTCACCA | | | | | | | TYYPDSVKGRFTIS | | | | | | |
| | | TCTCCAGAGAAAATGCCAAGAACTCCTTGTCTCTTCAAATG | | | | | | | RENAKNSLSLQMNS | | | | | | |
| | | AATAGCCTGAGAGCCGGGGACTCGGCTGTGTATTACTGTGC | | | | | | | LRAGDSAVYYCARG | | | | | | |
| | | AAGAGGGGAACAACGTATGATGATTTCTGGCGTGGTTCCGC | | | | | | | EQRMMISGVVPRYY | | | | | | |
| | | GTTACTATTATTACATGGACGTCTGGGGCAAAGGGACCACG | | | | | | | YYMDVWGKGTTVTV | | | | | | |
| | | GTCACCGTCTCCTCA | | | | | | | SS | | | | | | |
| ADI-81553 | 1287 | TGGGGCCTCAGTGAAGGTCTCCTGCAAGGCTTCTGGTTACA | | | | | | 6912 | QVQLVQSGAEVKKP | | 7265 | 8 | | 7 | |
| | | GGTTTACCAGCTACGGTATCAGCTGGGTGCGACAGGCCCCT | | | | | | | GASVKVSCKASGYR | | | | | | |
| | | GGACAAGGGCTTGAGTGGATGGGATGGATCAGCGTTTACAA | | | | | | | FTSYGISWVRQAPG | | | | | | |
| | | CGGTGACACAAATTATGCACAGAAGGTCCAGGGCAGAGTCA | | | | | | | QGLEWMGWISVYNG | | | | | | |
| | | CCATGACCACAGACACATCCACGAACACAGCCTACATGCAG | | | | | | | DTNYAQKVQGRVTM | | | | | | |
| | | TTGAGGAACCTAAGATCTGACGACACGGCCGTGTATTACTG | | | | | | | TTDTSTNTAYMQLR | | | | | | |
| | | TGCGCGAGACACTATATATATGGTTCGGGGAGTCCTTGATG | | | | | | | NLRSDDTAVYYCAR | | | | | | |
| | | CTTTTGATATCTGGGGCCAAGGGACCACGGTCACCGTCTCC | | | | | | | DTIYMVRGVLDAFD | | | | | | |
| | | TCA | | | | | | | IWGQGTTVTVSS | | | | | | |
| ADI-81636 | 1286 | TGGGTCCTCGGTGAAGGTCTCCTGCAAGGCTTCTGGAGGCA | | | | | | 6913 | EVQLVQSGAEVKKP | | 7266 | 8 | | 7 | |
| | | CCTTCAGCAGCTATACTTTCAGCTGGGTGCGACAGGCCCCT | | | | | | | GSSVKVSCKASGGT | | | | | | |
| | | GGACAAGGGCTTGAGTGGATGGGAAGGATCTTCCCCATCCT | | | | | | | FSSYTFSWVRQAPG | | | | | | |
| | | TGGTATACCAAACTACGCACAGAAGTTCCAGGACAGAGTCA | | | | | | | QGLEWMGRIFPILG | | | | | | |
| | | CCATTAGTGCGGACAGATCCACGAGCACAGCCTACATGGAA | | | | | | | IPNYAQKFQDRVTI | | | | | | |
| | | CTGAGCAGCCTGAGATCTGAAGACACGGCCGTGTATTTCTG | | | | | | | SADRSTSTAYMELS | | | | | | |
| | | TGCGAGAGACCGAGCAGCAGCTGACACGAACACCCTTCCCA | | | | | | | SLRSEDTAVYFCAR | | | | | | |
| | | CGTACTACTTTGACTACTGGGGCCAGGGAACCCTGGTCACC | | | | | | | DRAAADTNTLPTYY | | | | | | |
| | | GTCTCCTCA | | | | | | | FDYWGQGTLVTVSS | | | | | | |
| ADI-81536 | 6811 | CAGGTCACGTTGAAGGAGTCTGGTCCTGCGCTGGTGAAACC | | | | | | 6914 | QVTLKESGPALVKP | | 7267 | 6 | | 6 | |
| | | CAAACAGACCCTCACACTGACCTGCACCTTCTCTGGGTTCT | | | | | | | KQTLTLTCTFSGFS | | | | | | |
| | | CATTCAGCACTAGTGGAGTCGGTGTGGCCTGGATCCGTCAG | | | | | | | FSTSGVGVAWIRQP | | | | | | |
| | | CCCCCAGGAAAGGCCCTGGAGTGGCTTGCACTCATTTATTG | | | | | | | PGKALEWLALIYWD | | | | | | |
| | | GGATGGTGATAAGCGCTACAGCCCTTCTCTGAAGAGCAGGC | | | | | | | GDKRYSPSLKSRLT | | | | | | |
| | | TCACCATCACCAGGGACACCTCCAAAAACCAGGTGGTCCTT | | | | | | | ITRDTSKNQVVLTM | | | | | | |
| | | ACAATGACCAACATGGACCCTGTGGACACAGCCACATATTA | | | | | | | TNMDPVDTATYYCA | | | | | | |
| | | CTGTGCGCACCACGACATCCCGACGATTTTTAGTGTCTGGG | | | | | | | HHDIPTIFSVWGTG | | | | | | |
| | | GCACAGGGACCACGGTCACCGTCTCCTCA | | | | | | | TTVTVSS | | | | | | |
| ADI-81684 | 1291 | GAGGTGCAGCTGGTGGAGTCTGGGGGAGGCTTGGTACAGCC | | | | | | 6915 | EVQLVESGGGLVQP | | 7268 | 6 | | 6 | |
| | | TGGGGGGTCCCTGAGACTCTCCTGTGCAGCCTCTGGATTCA | | | | | | | GGSLRLSCAASGFT | | | | | | |
| | | CCTTCAGTACCTACGACATGCACTGGGTCCGCCAAGTTACA | | | | | | | FSTYDMHWVRQVTG | | | | | | |
| | | GGAAAAGGTCTGGAGTGGGTCTCAGCTATTGGTACTGGTGG | | | | | | | KGLEWVSAIGTGGH | | | | | | |
| | | TCACACATACTATCCAGACTCCGTGAAGGGCCGATTCACCA | | | | | | | TYYPDSVKGRFTIS | | | | | | |
| | | TCTCCAGAGAAAATGCCAAGAGCTCCTTGTATCTTCAAATG | | | | | | | RENAKSSLYLQMNS | | | | | | |
| | | AACAGCCTGAGAGCCGGGGACACGGCTGTGTATTACTGTGC | | | | | | | LRAGDTAVYYCARG | | | | | | |
| | | AAGAGGGCTTCACTCCGTAGCAGCAGGTGGCACGATTCGAT | | | | | | | LHSVAAGGTIRYYY | | | | | | |
| | | ACTACTACTACATGGACGTCTGGGGCAAAGGGACCACGGTC | | | | | | | YMDVWGKGTTVTVS | | | | | | |
| | | ACCGTCTCCTCA | | | | | | | S | | | | | | |
| ADI-81794 | 1286 | GAGGTGCAGCTGTTGGAGTCTGGAGCACAGGTGAAAAAGCC | | | | | | 6916 | EVQLLESGAQVKKP | | 7269 | 6 | | 6 | |
| | | CGGGGAGTCTCTGAAGATCTCCTGTAAGGGTTCTGGATACA | | | | | | | GESLKISCKGSGYS | | | | | | |
| | | GCTTTATCAGCAACTGGATCGGCTGGGTGCGCCAGATGCCC | | | | | | | FISNWIGWVRQMPG | | | | | | |
| | | GGGAAAGGCCTGGAGTGGATGGGGATCATCTATCCTGGTGA | | | | | | | KGLEWMGIIYPGDS | | | | | | |
| | | CTCTGATACTAGATACAGCCCGTCCTTCCAAGGCCAGGTCA | | | | | | | DTRYSPSFQGQVTM | | | | | | |
| | | CCATGTCAGTCGACAAGTCCATCAGCACCGCCTACCTGCAG | | | | | | | SVDKSISTAYLQWT | | | | | | |
| | | TGGACCAGCCTGAAGGCCTCGGACACCGCCATGTATTACTG | | | | | | | SLKASDTAMYYCAR | | | | | | |
| | | TGCGAGACTCGAGGTGGGAGGTAGGGATTATTGGGGCCAGG | | | | | | | LEVGGRDYWGQGTL | | | | | | |
| | | GAACCCTGGTCACCGTCTCCTCA | | | | | | | VTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81869 | 47 | IGHV3- | EVQLL | 5376 | FSFSSY | 241 | WVRQAP | 431 | VILDDG | 5943 | RFTISR | 777 | ARDRGFT | 6557 | WGQGTL |
| | | 30 | ESGGG | | AMH | | GKGLEW | | SNKFYA | | DNSKNT | | VTWFDF | | VTVSS |
| | | | VVQPG | | | | VA | | DSVKG | | LYLQMN | | | | |
| | | | RSLRL | | | | | | | | SLRAED | | | | |
| | | | SCVGS | | | | | | | | TAVYYC | | | | |
| | | | G | | | | | | | | | | | | |
| ADI-81659 | 32 | IGHV1- | EVQLM | 5377 | YTFTGY | 5583 | WVRQAP | 433 | WINPNS | 5944 | RVTMTR | 6253 | ARVYTRF | 6558 | WGQGTL |
| | | 2 | ESGAE | | YMH | | GQGLEW | | GGTNYA | | DTSIST | | SAWWFDP | | VTVSS |
| | | | VKKPG | | | | MG | | QKFQG | | AYMELS | | | | |
| | | | ASVKV | | | | | | | | RLRSDD | | | | |
| | | | SCKAS | | | | | | | | TAVYYC | | | | |
| | | | G | | | | | | | | | | | | |
| ADI-81861 | 39 | IGHV1- | EVQLL | 5378 | YTFINY | 5584 | WVRQAP | 5806 | LITPDG | 5945 | RVTMTS | 6254 | ARGGIVP | 6559 | WGQGTL |
| | | 46 | ESGAE | | YIH | | GQGFEW | | GSRTYA | | DTSTNT | | AAKENFD | | VTVSS |
| | | | VKKPG | | | | MG | | QKFQG | | VYMDLS | | F | | |
| | | | ASAKI | | | | | | | | SLRSAD | | | | |
| | | | SCTAS | | | | | | | | TAFYYC | | | | |
| | | | G | | | | | | | | | | | | |
| ADI-81707 | 77 | IGHV3- | EVQLV | 5379 | FVFSND | 5585 | WVRQPL | 5807 | GIGTSG | 5946 | RFTISR | 6255 | TRGISTS | 6560 | WGKGTT |
| | | 13 | ESGGG | | DMH | | EKGLEW | | DTYYAD | | EDAKNS | | SAGYHYY | | VTVSS |
| | | | LVQPG | | | | VA | | SVKG | | LYLQMN | | YMDV | | |
| | | | GSLRL | | | | | | | | SLRAGE | | | | |
| | | | SCTAS | | | | | | | | TAVYFC | | | | |
| | | | G | | | | | | | | | | | | |
| ADI-81584 | 45 | IGHV4- | QVQLQ | 56 | GSLISG | 5586 | WIRQRP | 512 | CMYYSV | 5947 | RVAISV | 6256 | ARGADHR | 6561 | WGQGTL |
| | | 31 | QSGPG | | GYYWN | | GKGLEW | | GTHYNP | | DKFKNQ | | YDSRWYA | | VTVSS |
| | | | LVKPS | | | | IG | | SLKS | | FSLRLT | | HETSKFD | | |
| | | | QTLSL | | | | | | | | SVTAAD | | S | | |
| | | | TCTVS | | | | | | | | TAVYYC | | | | |
| | | | G | | | | | | | | | | | | |
| ADI-81717 | 87 | IGHV4- | EVQLL | 5380 | GSVSSG | 5587 | WIRQPP | 5808 | HVFHSG | 5948 | RLTILV | 6257 | ARDQGSG | 6562 | WGQGTT |
| | | 61 | ESGPG | | SYYWN | | GKQLEW | | GTNFNP | | DTSKNQ | | WLDAFEV | | VTVSS |
| | | | LVKPS | | | | IG | | SLRS | | FSLKLR | | | | |
| | | | ETLSL | | | | | | | | SVTAAD | | | | |
| | | | TCSVS | | | | | | | | TAIYYC | | | | |
| | | | G | | | | | | | | | | | | |
| ADI-81726 | 3 | IGHV4- | EVQLV | 5381 | GSISSS | 5559 | WVRQSP | 5809 | EIYHVG | 5949 | RVTISV | 6258 | ARTPFGD | 6563 | WGKGTT |
| | | 4 | ESGPG | | NWWT | | EKGLEW | | ATNDNP | | DKSKNQ | | FYYMDV | | VTVSS |
| | | | LVKPS | | | | PR | | SLKS | | FSLKLT | | | | |
| | | | GTLSL | | | | | | | | SVTAAD | | | | |
| | | | TCSVS | | | | | | | | TAVYYC | | | | |
| | | | G | | | | | | | | | | | | |
| ADI-81897 | 72 | IGHV3- | EVQLV | 5362 | IIVSYN | 283 | WVRQAP | 475 | DIYPGG | 5950 | RFTISR | 6259 | ARDGYGE | 6564 | WGQGTL |
| | | 53 | ESGGG | | YMS | | GKGLDW | | STFYAD | | DSSKNT | | NVFDF | | VTVSS |
| | | | LIQPG | | | | AS | | SLKG | | LYLQMN | | | | |
| | | | GSLRL | | | | | | | | SLRVDD | | | | |
| | | | SCAAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81869 | 1286 | GAGGTGCAGCTGTTGGAGTCTGGGGGAGGCGTGGTCCAGCC | | | | | | 6917 | EVQLLESGGGVVQP | | 7270 | 6 | | 6 | |
| | | TGGGAGGTCCCTGAGACTCTCCTGTGTAGGCTCTGGATTCA | | | | | | | GRSLRLSCVGSGFS | | | | | | |
| | | GCTTCAGTAGCTATGCTATGCACTGGGTCCGCCAGGCTCCA | | | | | | | FSSYAMHWVRQAPG | | | | | | |
| | | GGCAAGGGGCTAGAGTGGGTGGCAGTTATATTAGATGATGG | | | | | | | KGLEWVAVILDDGS | | | | | | |
| | | AAGTAATAAATTCTACGCAGACTCCGTGAAGGGCCGATTCA | | | | | | | NKFYADSVKGRFTI | | | | | | |
| | | CCATCTCCAGAGACAATTCCAAGAACACGCTGTATCTGCAA | | | | | | | SRDNSKNTLYLQMN | | | | | | |
| | | ATGAACAGCCTGAGAGCTGAGGACACGGCTGTGTATTACTG | | | | | | | SLRAEDTAVYYCAR | | | | | | |
| | | TGCGAGAGACCGGGGATTTACGGTGACTTGGTTTGACTTCT | | | | | | | DRGFTVTWFDFWGQ | | | | | | |
| | | GGGGCCAGGGAACCCTGGTCACCGTCTCCTCA | | | | | | | GTLVTVSS | | | | | | |
| ADI-81659 | 1286 | GAGGTGCAGCTGATGGAGTCTGGGGCTGAGGTGAAGAAGCC | | | | | | 6918 | EVQLMESGAEVKKP | | 7271 | 0 | | 0 | |
| | | TGGGGCCTCAGTGAAGGTCTCCTGCAAGGCTTCTGGATACA | | | | | | | GASVKVSCKASGYT | | | | | | |
| | | CCTTCACCGGCTACTATATGCACTGGGTGCGACAGGCCCCT | | | | | | | FTGYYMHWVRQAPG | | | | | | |
| | | GGACAAGGGCTTGAGTGGATGGGATGGATCAACCCTAACAG | | | | | | | QGLEWMGWINPNSG | | | | | | |
| | | TGGTGGCACAAACTATGCACAGAAGTTTCAGGGCAGGGTCA | | | | | | | GTNYAQKFQGRVTM | | | | | | |
| | | CCATGACCAGGGACACGTCCATCAGCACAGCCTACATGGAG | | | | | | | TRDTSISTAYMELS | | | | | | |
| | | CTGAGCAGGCTGAGATCTGACGACACGGCCGTGTATTACTG | | | | | | | RLRSDDTAVYYCAR | | | | | | |
| | | TGCGAGAGTTTACACACGATTTTCAGCGTGGTGGTTCGACC | | | | | | | VYTRFSAWWFDPWG | | | | | | |
| | | CCTGGGGCCAGGGAACCCTGGTCACCGTCTCCTCA | | | | | | | QGTLVTVSS | | | | | | |
| ADI-81861 | 1286 | GAGGTGCAGCTGTTGGAGTCCGGGGCTGAGGTGAAGAAGCC | | | | | | 6919 | EVQLLESGAEVKKP | | 7272 | 20 | | 17 | |
| | | TGGGGCCTCGGCGAAGATTTCCTGCACGGCATCTGGATACA | | | | | | | GASAKISCTASGYT | | | | | | |
| | | CCTTCATCAATTACTATATACACTGGGTGCGACAGGCCCCT | | | | | | | FINYYIHWVRQAPG | | | | | | |
| | | GGACAAGGCTTTGAGTGGATGGGACTAATCACCCCTGATGG | | | | | | | QGFEWMGLITPDGG | | | | | | |
| | | TGGTAGTAGAACCTACGCACAGAAGTTCCAGGGCAGAGTCA | | | | | | | SRTYAQKFQGRVTM | | | | | | |
| | | CCATGACCAGCGACACGTCCACGAACACAGTCTACATGGAC | | | | | | | TSDTSTNTVYMDLS | | | | | | |
| | | CTGAGCAGCCTGAGATCTGCGGACACGGCCTTTTATTATTG | | | | | | | SLRSADTAFYYCAR | | | | | | |
| | | TGCGAGAGGGGGCATTGTACCAGCTGCCAAGGAGAACTTTG | | | | | | | GGIVPAAKENFDFW | | | | | | |
| | | ACTTCTGGGGCCAGGGAACCCTGGTCACCGTCTCCTCA | | | | | | | GQGTLVTVSS | | | | | | |
| ADI-81707 | 1291 | TGGGGGGTCCCTGAGACTCTCCTGTACAGCCTCGGGATTCG | | | | | | 6920 | EVQLVESGGGLVQP | | 7273 | 18 | | 15 | |
| | | TCTTCAGTAACGACGACATGCACTGGGTCCGCCAACCTCTA | | | | | | | GGSLRLSCTASGFV | | | | | | |
| | | GAAAAAGGTCTGGAATGGGTCGCAGGTATTGGTACTTCCGG | | | | | | | FSNDDMHWVRQPLE | | | | | | |
| | | TGACACATATTATGCAGACTCAGTGAAGGGCCGATTCACCA | | | | | | | KGLEWVAGIGTSGD | | | | | | |
| | | TCTCCAGAGAAGATGCCAAGAACTCCTTGTATCTTCAAATG | | | | | | | TYYADSVKGRFTIS | | | | | | |
| | | AACAGCCTGAGAGCCGGGGAAACGGCTGTATATTTCTGTAC | | | | | | | REDAKNSLYLQMNS | | | | | | |
| | | ACGGGGAATCAGTACGTCCAGCGCGGGCTATCACTATTACT | | | | | | | LRAGETAVYFCTRG | | | | | | |
| | | ATATGGACGTCTGGGGCAAAGGGACCACGGTCACCGTCTCC | | | | | | | ISTSSAGYHYYYMD | | | | | | |
| | | TCA | | | | | | | VWGKGTTVTVSS | | | | | | |
| ADI-81584 | 1286 | CAGGTGCAGCTGCAGCAGTCGGGCCCAGGACTGGTGAAGCC | | | | | | 6921 | QVQLQQSGPGLVKP | | 7274 | 16 | | 15 | |
| | | TTCACAGACCCTGTCCCTCACCTGCACTGTCTCTGGTGGCT | | | | | | | SQTLSLTCTVSGGS | | | | | | |
| | | CCCTCATCAGTGGTGGTTACTACTGGAACTGGATCCGCCAG | | | | | | | LISGGYYWNWIRQR | | | | | | |
| | | CGCCCAGGGAAGGGCCTGGAGTGGATTGGGTGCATGTATTA | | | | | | | PGKGLEWIGCMYYS | | | | | | |
| | | CAGTGTGGGGACCCACTACAACCCGTCCCTCAAGAGTCGAG | | | | | | | VGTHYNPSLKSRVA | | | | | | |
| | | TTGCCATTTCCGTAGACAAGTTTAAGAACCAGTTCTCCCTG | | | | | | | ISVDKFKNQFSLRL | | | | | | |
| | | AGGCTGACCTCTGTGACTGCCGCGGACACGGCCGTGTATTA | | | | | | | TSVTAADTAVYYCA | | | | | | |
| | | CTGTGCGAGAGGGGCAGACCACCGGTATGACAGCAGATGGT | | | | | | | RGADHRYDSRWYAH | | | | | | |
| | | ACGCCCACGAGACGTCCAAGTTTGACTCCTGGGGCCAGGGA | | | | | | | ETSKFDSWGQGTLV | | | | | | |
| | | ACCCTGGTCACCGTCTCCTCA | | | | | | | TVSS | | | | | | |
| ADI-81717 | 1287 | GAGGTGCAGCTGTTGGAGTCGGGCCCAGGACTGGTGAAGCC | | | | | | 6922 | EVQLLESGPGLVKP | | 7275 | 16 | | 14 | |
| | | TTCGGAGACCCTGTCCCTCACCTGCAGTGTCTCTGGTGGCT | | | | | | | SETLSLTCSVSGGS | | | | | | |
| | | CCGTCAGCAGTGGTAGTTACTACTGGAACTGGATCCGGCAG | | | | | | | VSSGSYYWNWIRQP | | | | | | |
| | | CCCCCAGGGAAACAATTGGAGTGGATCGGCCATGTCTTTCA | | | | | | | PGKQLEWIGHVFHS | | | | | | |
| | | CAGTGGGGGCACCAACTTCAACCCCTCCCTCAGGAGTCGAC | | | | | | | GGTNFNPSLRSRLT | | | | | | |
| | | TCACCATATTAGTAGACACGTCCAAGAACCAGTTCTCCCTG | | | | | | | ILVDTSKNQFSLKL | | | | | | |
| | | AAGTTGAGGTCTGTGACCGCTGCGGACACGGCCATTTATTA | | | | | | | RSVTAADTAIYYCA | | | | | | |
| | | CTGTGCGAGAGATCAGGGTAGTGGCTGGCTAGATGCTTTTG | | | | | | | RDQGSGWLDAFEVW | | | | | | |
| | | AAGTCTGGGGCCAAGGGACCACGGTCACCGTCTCCTCA | | | | | | | GQGTTVTVSS | | | | | | |
| ADI-81726 | 1291 | GAGGTGCAGCTGGTGGAGTCGGGTCCAGGACTGGTGAAGCC | | | | | | 6923 | EVQLVESGPGLVKP | | 7276 | 16 | | 10 | |
| | | TTCGGGGACCCTGTCCCTCACTTGTTCTGTCTCTGGTGGCT | | | | | | | SGTLSLTCSVSGGS | | | | | | |
| | | CCATCAGTAGTAGTAACTGGTGGACTTGGGTCCGCCAGTCC | | | | | | | ISSSNWWTWVRQSP | | | | | | |
| | | CCAGAGAAGGGGCTGGAGTGGCCCCGTGAGATCTATCATGT | | | | | | | EKGLEWPREIYHVG | | | | | | |
| | | TGGGGCCACCAACGACAACCCGTCCCTCAAGAGTCGGGTCA | | | | | | | ATNDNPSLKSRVTI | | | | | | |
| | | CCATATCAGTCGACAAGTCCAAGAACCAATTCTCCCTGAAG | | | | | | | SVDKSKNQFSLKLT | | | | | | |
| | | TTGACTTCTGTGACCGCCGCGGACACGGCCGTGTATTACTG | | | | | | | SVTAADTAVYYCAR | | | | | | |
| | | TGCGAGGACCCCTTTTGGGGACTTCTACTACATGGACGTCT | | | | | | | TPFGDFYYMDVWGK | | | | | | |
| | | GGGGCAAGGGGACCACGGTCACCGTCTCCTCA | | | | | | | GTTVTVSS | | | | | | |
| ADI-81897 | 1286 | GAGGTGCAGCTGGTGGAGTCTGGGGGAGGCTTGATCCAGCC | | | | | | 6924 | EVQLVESGGGLIQP | | 7277 | 15 | | 12 | |
| | | TGGGGGGTCCCTGAGACTCTCCTGTGCAGCCTCTGGGATCA | | | | | | | GGSLRLSCAASGII | | | | | | |
| | | TCGTCAGTTATAACTATATGAGTTGGGTTCGCCAGGCTCCA | | | | | | | VSYNYMSWVRQAPG | | | | | | |
| | | GGGAAGGGACTGGACTGGGCCTCAGATATTTATCCCGGTGG | | | | | | | KGLDWASDIYPGGS | | | | | | |
| | | TAGCACATTCTACGCAGACTCCCTGAAGGGCCGATTCACCA | | | | | | | TFYADSLKGRFTIS | | | | | | |
| | | TCTCCAGAGACAGTTCCAAGAACACGTTGTATCTTCAAATG | | | | | | | RDSSKNTLYLQMNS | | | | | | |
| | | AACAGCCTGAGAGTCGACGACACGGCCGTGTATTACTGTGC | | | | | | | LRVDDTAVYYCARD | | | | | | |
| | | GAGAGATGGGTACGGTGAAAATGTCTTTGATTTTTGGGGCC | | | | | | | GYGENVFDFWGQGT | | | | | | |
| | | AGGGAACCCTGGTCACCGTCTCCTCA | | | | | | | LVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81718 | 88 | IGHV5- | EVQLV | 5382 | YSFTTY | 5515 | WVRQMP | 476 | NIYPID | 5951 | QVTISA | 6260 | SRQGSGT | 6565 | WGQGAL |
| | | 51 | QSGAE | | WIG | | GKGLEW | | SDTRYN | | DKSITT | | YYDFDY | | VTVSS |
| | | | VKKPG | | | | MG | | PSFQG | | AYLHWN | | | | |
| | | | ESLTI | | | | | | | | SLKASD | | | | |
| | | | SCKAS | | | | | | | | TAIYFC | | | | |
| | | | G | | | | | | | | | | | | |
| ADI-81770 | 46 | IGHV3- | EVQLL | 5383 | FTVSDY | 5588 | WVRQAP | 470 | VIYSGN | 5952 | RFTLSR | 6261 | AKGADTW | 6566 | WGQGTL |
| | | 53 | ETGGG | | YMS | | GKGLEW | | NTYYID | | DTSKNT | | YGGTFDS | | VTVSS |
| | | | LMQPG | | | | IS | | SVKG | | LFLQMN | | | | |
| | | | GSLRL | | | | | | | | SLRAED | | | | |
| | | | SCAAS | | | | | | | | TAVYYC | | | | |
| | | | G | | | | | | | | | | | | |
| ADI-81772 | 48 | IGHV3- | EAQLV | 5384 | FTFEDY | 5589 | WVRQAP | 437 | GISWNG | 752 | RFYISR | 6262 | AKDLYLK | 6567 | WGQGTT |
| | | 9 | ESGGS | | TMH | | GKGLEW | | GIIGYA | | DNAQNS | | EGYNSGG | | VTVSS |
| | | | LVQPG | | | | VS | | DSVKG | | LYLQML | | AFDM | | |
| | | | RSLRL | | | | | | | | RLRPED | | | | |
| | | | SCAAS | | | | | | | | | | | | |
| | | | G | | | | | | | | TALYYC | | | | |
| ADI-81834 | 13 | IGHV1- | QVQLV | 50 | GTFNNH | 342 | WVRRAP | 488 | KIIPII | 677 | RVTITA | 926 | ARRTSSS | 1177 | WGQGTL |
| | | 69 | QSGAE | | FIN | | GQGLEW | | DIANSA | | DKSTST | | SEYFDS | | VTVSS |
| | | | VKKPG | | | | MG | | QKFQG | | VYMELS | | | | |
| | | | SSVKV | | | | | | | | SLRSDD | | | | |
| | | | SCKAS | | | | | | | | TAMYYC | | | | |
| | | | G | | | | | | | | | | | | |
| ADI-81757 | 33 | IGHV1- | EVQLV | 5385 | HTFTLH | 5590 | WVRQAP | 433 | IINPDA | 5953 | RVSMTR | 6263 | ARGALVP | 6568 | WGQGTT |
| | | 46 | ESGAE | | YMH | | GQGLEW | | GSTTYA | | DTSTST | | DAHDALD | | VTVSS |
| | | | VKKPG | | | | MG | | QKFQG | | VYLHLS | | I | | |
| | | | ASVKV | | | | | | | | SLRSED | | | | |
| | | | SCKAS | | | | | | | | TAVYYC | | | | |
| | | | G | | | | | | | | | | | | |
| ADI-81835 | 14 | IGHV3- | EVQLL | 5354 | IIVSRN | 291 | WVRQAP | 437 | VVYPGG | 5954 | RFTISR | 6264 | ARQDNND | 6569 | WGQGTT |
| | | 53 | ETGGG | | YMT | | GKGLEW | | STFYAD | | DHSKNT | | AFDI | | VTVSS |
| | | | LIQPG | | | | VS | | SVRG | | LYLQVT | | | | |
| | | | GSLRL | | | | | | | | SLRAED | | | | |
| | | | SCAAS | | | | | | | | TAVYYC | | | | |
| | | | E | | | | | | | | | | | | |
| ADI-81845 | 23 | IGHV3- | EVQLV | 5386 | FSFGTY | 5591 | WVRQST | 5810 | AIGTSG | 5930 | RFTISR | 6265 | VRGTTVT | 6570 | WGQGTL |
| | | 13 | ESGGK | | DMH | | GKGLEW | | DTYYAD | | EDAKKS | | GGLEYYF | | VTVSS |
| | | | LVQPG | | | | VS | | SVKG | | LYLQMN | | | | |
| | | | GSLRL | | | | | | | | SLRAGD | | | | |
| | | | SCAAS | | | | | | | | TAVYYC | | | | |
| | | | G | | | | | | | | | | | | |
| ADI-81860 | 38 | IGHV3- | EVQLV | 5387 | FTFSSY | 199 | WVRQAP | 5811 | LISNDG | 5955 | RFTASR | 6266 | ARDGPMT | 6571 | WGQGTL |
| | | 30 | ESGGG | | AMH | | GSGLEW | | RMKFYT | | DNSKNT | | TVTCFDY | | VTVSS |
| | | | VVQPG | | | | VA | | DSVKG | | LYLEMN | | | | |
| | | | RSLRL | | | | | | | | SLRSED | | | | |
| | | | SCATS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81718 | 1285 | GAGGTGCAGCTGGTGCAGTCTGGAGCAGAGGTGAAAAAGCC | | | | | | 6925 | EVQLVQSGAEVKKP | | 7278 | 14 | | 11 | |
| | | CGGGGAGTCTCTGACGATCTCCTGTAAGGCTTCTGGATACA | | | | | | | GESLTISCKASGYS | | | | | | |
| | | GCTTTACCACCTACTGGATCGGCTGGGTGCGCCAGATGCCC | | | | | | | FTTYWIGWVRQMPG | | | | | | |
| | | GGAAAAGGCCTGGAGTGGATGGGGAACATCTATCCTATTGA | | | | | | | KGLEWMGNIYPIDS | | | | | | |
| | | CTCTGATACCAGATATAATCCGTCCTTCCAAGGCCAGGTCA | | | | | | | DTRYNPSFQGQVTI | | | | | | |
| | | CCATCTCGGCCGACAAGTCCATCACCACCGCCTATCTGCAC | | | | | | | SADKSITTAYLHWN | | | | | | |
| | | TGGAACAGCCTGAAGGCCTCGGACACCGCCATTTATTTTTG | | | | | | | SLKASDTAIYFCSR | | | | | | |
| | | TTCGAGACAAGGTAGTGGGACCTACTACGACTTTGACTACT | | | | | | | QGSGTYYDFDYWGQ | | | | | | |
| | | GGGGCCAGGGAGCCCTGGTCACCGTCTCCTCA | | | | | | | GALVTVSS | | | | | | |
| ADI-81770 | 1286 | GAGGTGCAGCTGTTGGAGACTGGAGGAGGCTTGATGCAGCC | | | | | | 6926 | EVQLLETGGGLMQP | | 7279 | 14 | | 10 | |
| | | TGGGGGGTCCCTGAGACTCTCCTGTGCAGCCTCTGGGTTCA | | | | | | | GGSLRLSCAASGFT | | | | | | |
| | | CCGTCAGTGACTACTACATGAGCTGGGTCCGCCAGGCTCCA | | | | | | | VSDYYMSWVRQAPG | | | | | | |
| | | GGGAAGGGGCTGGAGTGGATTTCAGTTATTTATAGTGGAAA | | | | | | | KGLEWISVIYSGNN | | | | | | |
| | | TAACACCTACTACATAGACTCCGTGAAGGGCCGATTCACCC | | | | | | | TYYIDSVKGRFTLS | | | | | | |
| | | TCTCCAGAGACACTTCCAAGAACACGCTGTTTCTCCAAATG | | | | | | | RDTSKNTLFLQMNS | | | | | | |
| | | AACAGCCTGAGAGCCGAAGACACGGCCGTGTATTACTGTGC | | | | | | | LRAEDTAVYYCAKG | | | | | | |
| | | AAAAGGGGCCGACACTTGGTACGGGGGGACCTTTGACTCCT | | | | | | | ADTWYGGTFDSWGQ | | | | | | |
| | | GGGGCCAGGGAACCCTGGTCACCGTCTCCTCA | | | | | | | GTLVTVSS | | | | | | |
| ADI-81772 | 1287 | TGGCAGGTCCCTGAGACTCTCCTGTGCAGCCTCTGGATTCA | | | | | | 6927 | EAQLVESGGSLVQP | | 7280 | 14 | | 10 | |
| | | CCTTTGAAGATTATACCATGCACTGGGTCCGGCAAGCTCCA | | | | | | | GRSLRLSCAASGFT | | | | | | |
| | | GGGAAGGGCCTGGAGTGGGTCTCAGGTATAAGTTGGAATGG | | | | | | | FEDYTMHWVRQAPG | | | | | | |
| | | TGGTATAATCGGCTATGCGGACTCTGTGAAGGGCCGATTCT | | | | | | | KGLEWVSGISWNGG | | | | | | |
| | | ACATCTCCAGAGACAACGCCCAGAACTCACTTTATCTGCAA | | | | | | | IIGYADSVKGRFYI | | | | | | |
| | | ATGCTCCGTCTAAGACCTGAGGACACGGCCTTGTATTACTG | | | | | | | SRDNAQNSLYLQML | | | | | | |
| | | TGCAAAAGACTTATATCTTAAAGAAGGCTACAATTCGGGAG | | | | | | | RLRPEDTALYYCAK | | | | | | |
| | | GCGCTTTTGATATGTGGGGCCAAGGGACAACGGTCACCGTC | | | | | | | DLYLKEGYNSGGAF | | | | | | |
| | | TCCTCA | | | | | | | DMWGQGTTVTVSS | | | | | | |
| ADI-81834 | 1286 | CAGGTCCAGCTGGTACAGTCTGGGGCTGAGGTGAAGAAGCC | | | | | | 6928 | QVQLVQSGAEVKKP | | 1757 | 14 | | 13 | |
| | | TGGGTCCTCGGTGAAGGTCTCCTGCAAGGCTTCTGGAGGCA | | | | | | | GSSVKVSCKASGGT | | | | | | |
| | | CCTTCAACAACCATTTTATCAACTGGGTGCGACGGGCCCCT | | | | | | | FNNHFINWVRRAPG | | | | | | |
| | | GGACAAGGGCTTGAGTGGATGGGAAAGATCATCCCTATCAT | | | | | | | QGLEWMGKIIPIID | | | | | | |
| | | TGATATAGCAAACTCCGCACAGAAGTTCCAGGGCAGAGTCA | | | | | | | IANSAQKFQGRVTI | | | | | | |
| | | CGATTACCGCGGACAAATCCACGAGCACAGTCTACATGGAG | | | | | | | TADKSTSTVYMELS | | | | | | |
| | | CTGAGCAGCCTGAGATCTGATGACACGGCCATGTATTACTG | | | | | | | SLRSDDTAMYYCAR | | | | | | |
| | | TGCGCGTCGAACTAGCAGTTCGTCTGAGTATTTTGACTCCT | | | | | | | RTSSSSEYFDSWGQ | | | | | | |
| | | GGGGCCAGGGAACCCTGGTCACCGTCTCCTCA | | | | | | | GTLVTVSS | | | | | | |
| ADI-81757 | 1287 | GAGGTGCAGCTGGTGGAGTCTGGGGCTGAGGTGAAGAAGCC | | | | | | 6929 | EVQLVESGAEVKKP | | 7281 | 13 | | 9 | |
| | | TGGGGCCTCAGTGAAGGTTTCCTGCAAGGCATCTGGACACA | | | | | | | GASVKVSCKASGHT | | | | | | |
| | | CCTTCACCCTCCACTATATGCACTGGGTGCGACAGGCCCCT | | | | | | | FTLHYMHWVRQAPG | | | | | | |
| | | GGACAAGGACTTGAGTGGATGGGAATAATCAACCCTGATGC | | | | | | | QGLEWMGIINPDAG | | | | | | |
| | | TGGTAGCACAACATACGCACAGAAGTTCCAGGGCAGAGTCT | | | | | | | STTYAQKFQGRVSM | | | | | | |
| | | CCATGACCAGGGACACGTCCACGAGCACAGTCTACTTGCAC | | | | | | | TRDTSTSTVYLHLS | | | | | | |
| | | CTGAGCAGCCTGAGATCTGAGGACACGGCCGTGTATTACTG | | | | | | | SLRSEDTAVYYCAR | | | | | | |
| | | TGCGAGAGGGGCACTAGTACCAGATGCCCATGATGCTCTTG | | | | | | | GALVPDAHDALDIW | | | | | | |
| | | ATATTTGGGGCCAGGGGACCACGGTCACCGTCTCCTCA | | | | | | | GQGTTVTVSS | | | | | | |
| ADI-81835 | 1287 | GAGGTGCAGCTGTTGGAGACTGGAGGAGGCTTGATCCAGCC | | | | | | 6930 | EVQLLETGGGLIQP | | 7282 | 13 | | 12 | |
| | | GGGGGGGTCCCTGAGACTCTCCTGTGCAGCCTCTGAGATCA | | | | | | | GGSLRLSCAASEII | | | | | | |
| | | TCGTCAGTAGGAACTACATGACCTGGGTCCGCCAGGCTCCA | | | | | | | VSRNYMTWVRQAPG | | | | | | |
| | | GGCAAAGGGCTGGAGTGGGTCTCAGTTGTTTATCCCGGTGG | | | | | | | KGLEWVSVVYPGGS | | | | | | |
| | | GAGCACTTTCTACGCAGACTCCGTGAGGGGCCGATTCACCA | | | | | | | TFYADSVRGRFTIS | | | | | | |
| | | TCTCCAGAGACCATTCCAAGAACACTCTTTATCTTCAAGTG | | | | | | | RDHSKNTLYLQVTS | | | | | | |
| | | ACCAGCCTGAGAGCCGAGGACACGGCCGTGTATTACTGTGC | | | | | | | LRAEDTAVYYCARQ | | | | | | |
| | | GAGACAAGACAATAATGATGCTTTTGATATTTGGGGGCAAG | | | | | | | DNNDAFDIWGQGTT | | | | | | |
| | | GGACCACGGTCACCGTCTCCTCA | | | | | | | VTVSS | | | | | | |
| ADI-81845 | 1286 | GAGGTGCAGCTGGTGGAGTCTGGGGGAAAGTTGGTACAGCC | | | | | | 6931 | EVQLVESGGKLVQP | | 7283 | 13 | | 10 | |
| | | TGGGGGGTCCCTGAGACTCTCCTGTGCAGCCTCTGGATTCA | | | | | | | GGSLRLSCAASGFS | | | | | | |
| | | GTTTCGGTACCTACGACATGCACTGGGTCCGCCAATCTACA | | | | | | | FGTYDMHWVRQSTG | | | | | | |
| | | GGAAAAGGTCTGGAGTGGGTCTCAGCTATTGGTACTTCTGG | | | | | | | KGLEWVSAIGTSGD | | | | | | |
| | | TGACACATACTATGCAGACTCCGTGAAGGGCCGATTCACCA | | | | | | | TYYADSVKGRFTIS | | | | | | |
| | | TCTCTAGAGAAGATGCCAAGAAGTCTTTGTATCTTCAAATG | | | | | | | REDAKKSLYLQMNS | | | | | | |
| | | AACAGTCTGAGAGCCGGGGACACGGCTGTGTATTATTGTGT | | | | | | | LRAGDTAVYYCVRG | | | | | | |
| | | AAGAGGTACTACGGTGACTGGGGGACTGGAATACTACTTTG | | | | | | | TTVTGGLEYYFDYW | | | | | | |
| | | ACTACTGGGGCCAGGGAACCCTGGTCACCGTCTCCTCA | | | | | | | GQGTLVTVSS | | | | | | |
| ADI-81860 | 1286 | GAGGTGCAGCTGGTGGAGTCTGGGGGAGGCGTGGTCCAGCC | | | | | | 6932 | EVQLVESGGGVVQP | | 7284 | 13 | | 10 | |
| | | TGGGAGGTCCCTGAGACTCTCCTGCGCAACCTCTGGATTCA | | | | | | | GRSLRLSCATSGFT | | | | | | |
| | | CCTTCAGTAGTTATGCTATGCACTGGGTCCGCCAGGCTCCA | | | | | | | FSSYAMHWVRQAPG | | | | | | |
| | | GGCTCGGGACTGGAGTGGGTGGCACTTATATCAAATGATGG | | | | | | | SGLEWVALISNDGR | | | | | | |
| | | AAGAATGAAATTCTACACAGACTCCGTGAAGGGCCGCTTCA | | | | | | | MKFYTDSVKGRFTA | | | | | | |
| | | CCGCCTCCAGAGACAATTCCAAGAACACTCTGTATCTGGAA | | | | | | | SRDNSKNTLYLEMN | | | | | | |
| | | ATGAACAGCCTGAGATCTGAAGACACGGCTGTGTATTACTG | | | | | | | SLRSEDTAVYYCAR | | | | | | |
| | | TGCGAGAGATGGCCCTATGACTACGGTGACTTGCTTTGACT | | | | | | | DGPMTTVTCFDYWG | | | | | | |
| | | ACTGGGGCCAGGGAACCCTGGTCACCGTCTCCTCA | | | | | | | QGTLVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81590 | 35 | IGHV5- | QVQLV | 5388 | YSFISN | 5582 | WVRQMP | 476 | IIWPGD | 5956 | QVTISV | 6267 | VRRGSAW | 6572 | WGQGTL |
| | | 51 | QSGAE | | WIG | | GKGLEW | | SDTTYS | | DKSITT | | EIDY | | VTVSS |
| | | | VKKPG | | | | MG | | PSFQG | | AYLEWG | | | | |
| | | | ESLKI | | | | | | | | SLKASD | | | | |
| | | | SCKTS | | | | | | | | TAIYYC | | | | |
| | | | G | | | | | | | | | | | | |
| ADI-81708 | 78 | IGHV3- | EVQLL | 5355 | FPFSSY | 5592 | WVRQAT | 467 | ATGTSG | 5957 | RFTISR | 6268 | VRGDVVE | 6573 | WGKGTT |
| | | 13 | ESGGG | | DMH | | GKGLEW | | DTSYLD | | DDARSS | | VPSVPSY | | VTVSS |
| | | | LVQPG | | | | VS | | SVKG | | LYLQMN | | YYYYYMD | | |
| | | | GSLRL | | | | | | | | NLRAGD | | V | | |
| | | | SCAAS | | | | | | | | TAVYYC | | | | |
| | | | G | | | | | | | | | | | | |
| ADI-81737 | 14 | IGHV5- | EVQLL | 5389 | DYFSTY | 5593 | WVRQMP | 5812 | IFFPGD | 5958 | QVTMSA | 6269 | ARATYCG | 6574 | WGQGTM |
| | | 51 | ESGAE | | WIG | | GKGLEW | | SDARYS | | DKSIST | | GDCSSGG | | VTVSS |
| | | | VKKPG | | | | VG | | PSFQG | | AYLQWS | | AFDV | | |
| | | | ESLKI | | | | | | | | SLKASD | | | | |
| | | | SCQDS | | | | | | | | TAMYYC | | | | |
| | | | G | | | | | | | | | | | | |
| ADI-81905 | 79 | IGHV4- | EVQLL | 5346 | ASISRS | 5594 | WIRQHP | 450 | NIYNSG | 5959 | RLTISV | 6270 | ARDYGGN | 6575 | WGQGTL |
| | | 31 | ESGPG | | GFYWN | | GKGLEW | | STYYNP | | DTSKNQ | | AQYFHY | | VTVSS |
| | | | LVKPS | | | | IG | | SLKS | | FSLKLN | | | | |
| | | | QTLSL | | | | | | | | SVTAAD | | | | |
| | | | TCTVS | | | | | | | | TAVYYC | | | | |
| | | | G | | | | | | | | | | | | |
| ADI-81618 | 75 | IGHV4- | PGAAG | 5390 | GSLSSG | 5595 | WIRQHP | 450 | YISYTG | 5960 | PVTMSV | 6271 | ARASMTP | 6576 | WGKGTT |
| | | 31 | GVGPG | | GYYWN | | GKGLEW | | STDYNP | | DTSKSQ | | VTQAGPL | | VTVSS |
| | | | LVKTS | | | | IG | | SLKS | | FSLRLS | | GYYYMDV | | |
| | | | QTLSL | | | | | | | | SVTAAD | | | | |
| | | | TCTVS | | | | | | | | SAVYYC | | | | |
| | | | G | | | | | | | | | | | | |
| ADI-81901 | 59 | IGHV3- | EVQLV | 51 | FSFSTY | 5596 | WVRQAP | 443 | DISGDG | 5961 | RFTITR | 6272 | VRDVSVD | 6577 | WGQGTL |
| | | 64D | ESGGG | | SMH | | GKGLEY | | LITYYA | | DNSKNT | | TSMVTVF | | VTVSS |
| | | | LVQPG | | | | VS | | DSVKG | | LYLHMS | | EY | | |
| | | | GSLRL | | | | | | | | SLRAED | | | | |
| | | | SCSAS | | | | | | | | TAVYYC | | | | |
| | | | G | | | | | | | | | | | | |
| ADI-81532 | 1 | IGHV3- | QVQLV | 5391 | FIFSSY | 5597 | WVRQVT | 5813 | AIGTAG | 5962 | RFTISR | 6273 | ARGDQPI | 6578 | WGKGTT |
| | | 13 | QSGGG | | DMH | | GKGLEW | | DTYYED | | ENAKNS | | TISGVVE | | VTVSS |
| | | | LAQPG | | | | VA | | SVKG | | VYLQMN | | TYYMDV | | |
| | | | GSLRL | | | | | | | | SLRAGD | | | | |
| | | | SCAAS | | | | | | | | TAMYYC | | | | |
| | | | G | | | | | | | | | | | | |
| ADI-81537 | 1 | IGHV3- | QVQLV | 5391 | FIFSSY | 5597 | WVRQVT | 5813 | AIGTAG | 5962 | RFTISR | 6273 | ARGDQPI | 6578 | WGKGTT |
| | | 13 | QSGGG | | DMH | | GKGLEW | | DTYYED | | ENAKNS | | TISGVVE | | VTVSS |
| | | | LAQPG | | | | VA | | SVKG | | VYLQMN | | TYYMDV | | |
| | | | GSLRL | | | | | | | | SLRAGD | | | | |
| | | | SCAAS | | | | | | | | TAMYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81590 | 1286 | CAGGTGCAGCTGGTGCAGTCTGGAGCAGAGGTGAAAAAGCC | | | | | | 6933 | QVQLVQSGAEVKKP | | 7285 | 12 | | 10 | |
| | | GGGGGAGTCTCTGAAGATCTCCTGTAAGACTTCTGGATACA | | | | | | | GESLKISCKTSGYS | | | | | | |
| | | GCTTTATCAGCAACTGGATCGGCTGGGTGCGCCAGATGCCC | | | | | | | FISNWIGWVRQMPG | | | | | | |
| | | GGGAAAGGCCTGGAGTGGATGGGGATCATCTGGCCTGGTGA | | | | | | | KGLEWMGIIWPGDS | | | | | | |
| | | CTCTGATACCACATACAGCCCGTCCTTCCAAGGCCAGGTCA | | | | | | | DTTYSPSFQGQVTI | | | | | | |
| | | CCATCTCAGTCGACAAGTCCATCACCACCGCCTACCTGGAG | | | | | | | SVDKSITTAYLEWG | | | | | | |
| | | TGGGGCAGCCTGAAGGCCTCGGACACCGCCATATACTACTG | | | | | | | SLKASDTAIYYCVR | | | | | | |
| | | TGTGAGACGAGGCAGTGCCTGGGAAATTGACTACTGGGGCC | | | | | | | RGSAWEIDYWGQGT | | | | | | |
| | | AGGGAACCCTGGTCACCGTCTCCTCA | | | | | | | LVTVSS | | | | | | |
| ADI-81708 | 1291 | GAGGTGCAGCTGTTGGAGTCTGGGGGAGGCTTGGTGCAGCC | | | | | | 6934 | EVQLLESGGGLVQP | | 7286 | 12 | | 11 | |
| | | GGGGGGGTCCCTGAGACTCTCCTGTGCAGCCTCTGGGTTCC | | | | | | | GGSLRLSCAASGFP | | | | | | |
| | | CCTTCAGTAGCTACGACATGCACTGGGTCCGCCAAGCTACA | | | | | | | FSSYDMHWVRQATG | | | | | | |
| | | GGAAAAGGTCTGGAGTGGGTCTCAGCCACTGGTACTTCTGG | | | | | | | KGLEWVSATGTSGD | | | | | | |
| | | TGACACATCCTATTTAGACTCCGTGAAGGGCCGATTCACCA | | | | | | | TSYLDSVKGRFTIS | | | | | | |
| | | TCTCCAGAGACGATGCCAGGAGCTCCTTGTATCTTCAAATG | | | | | | | RDDARSSLYLQMNN | | | | | | |
| | | AACAACCTGAGAGCCGGGGACACGGCTGTGTATTACTGTGT | | | | | | | LRAGDTAVYYCVRG | | | | | | |
| | | AAGAGGTGATGTTGTAGAAGTACCATCTGTCCCGTCCTACT | | | | | | | DVVEVPSVPSYYYY | | | | | | |
| | | ACTACTACTACTACATGGACGTCTGGGGCAAAGGGACCACG | | | | | | | YYMDVWGKGTTVTV | | | | | | |
| | | GTCACCGTCTCCTCA | | | | | | | SS | | | | | | |
| ADI-81737 | 1299 | CGGGGAGTCTCTGAAGATCTCCTGTCAGGATTCTGGAGACT | | | | | | 6935 | EVQLLESGAEVKKP | | 7287 | 12 | | 11 | |
| | | ACTTTTCCACCTACTGGATCGGCTGGGTGCGCCAGATGCCC | | | | | | | GESLKISCQDSGDY | | | | | | |
| | | GGGAAAGGCCTGGAGTGGGTGGGGATCTTCTTTCCTGGTGA | | | | | | | FSTYWIGWVRQMPG | | | | | | |
| | | CTCTGATGCCAGATACAGCCCGTCCTTCCAAGGCCAGGTCA | | | | | | | KGLEWVGIFFPGDS | | | | | | |
| | | CCATGTCAGCCGACAAGTCCATCAGCACCGCCTACCTGCAG | | | | | | | DARYSPSFQGQVTM | | | | | | |
| | | TGGAGCAGCCTGAAGGCCTCGGACACCGCCATGTATTACTG | | | | | | | SADKSISTAYLQWS | | | | | | |
| | | TGCGAGAGCAACATATTGTGGTGGTGATTGCTCTTCGGGCG | | | | | | | SLKASDTAMYYCAR | | | | | | |
| | | GTGCTTTTGATGTCTGGGGCCAAGGGACAATGGTCACCGTC | | | | | | | ATYCGGDCSSGGAF | | | | | | |
| | | TCCTCA | | | | | | | DVWGQGTMVTVSS | | | | | | |
| ADI-81905 | 1286 | GAGGTGCAGCTGTTGGAGTCGGGCCCAGGACTGGTGAAGCC | | | | | | 6936 | EVQLLESGPGLVKP | | 7288 | 12 | | 10 | |
| | | TTCACAGACCCTGTCCCTCACCTGCACTGTCTCTGGTGCTT | | | | | | | SQTLSLTCTVSGAS | | | | | | |
| | | CCATCAGCAGAAGTGGTTTCTACTGGAACTGGATCCGCCAG | | | | | | | ISRSGFYWNWIRQH | | | | | | |
| | | CACCCAGGGAAGGGCCTGGAGTGGATTGGGAACATCTATAA | | | | | | | PGKGLEWIGNIYNS | | | | | | |
| | | TAGTGGGAGCACCTACTACAACCCGTCCCTCAAGAGTCGAC | | | | | | | GSTYYNPSLKSRLT | | | | | | |
| | | TTACCATTTCAGTCGACACGTCTAAGAACCAGTTCTCCCTG | | | | | | | ISVDTSKNQFSLKL | | | | | | |
| | | AAGCTGAACTCTGTGACTGCCGCGGACACGGCCGTGTATTA | | | | | | | NSVTAADTAVYYCA | | | | | | |
| | | CTGTGCGAGAGACTACGGTGGTAACGCCCAATACTTTCACT | | | | | | | RDYGGNAQYFHYWG | | | | | | |
| | | ACTGGGGCCAGGGAACCCTGGTCACCGTCTCCTCA | | | | | | | QGTLVTVSS | | | | | | |
| ADI-81618 | 1291 | CCAGGTGCAGCTGGCGGAGTCGGCCCAGGACTGGTGAAGAC | | | | | | 6937 | PGAAGGVGPGLVKT | | 7289 | 11 | | 11 | |
| | | TTCACAGACCCTGTCCCTCACCTGCACTGTCTCTGGTGGCT | | | | | | | SQTLSLTCTVSGGS | | | | | | |
| | | CCCTCAGCAGTGGTGGTTACTACTGGAACTGGATCCGCCAG | | | | | | | LSSGGYYWNWIRQH | | | | | | |
| | | CACCCAGGGAAGGGCCTGGAGTGGATTGGGTACATCTCTTA | | | | | | | PGKGLEWIGYISYT | | | | | | |
| | | CACTGGGAGCACCGACTACAACCCGTCCCTCAAGAGTCCAG | | | | | | | GSTDYNPSLKSPVT | | | | | | |
| | | TTACCATGTCCGTAGACACGTCTAAGAGCCAGTTCTCCCTT | | | | | | | MSVDTSKSQFSLRL | | | | | | |
| | | AGGCTGAGCTCTGTGACTGCCGCGGACTCGGCCGTGTATTA | | | | | | | SSVTAADSAVYYCA | | | | | | |
| | | CTGTGCGAGAGCCTCAATGACTCCGGTGACTCAGGCAGGTC | | | | | | | RASMTPVTQAGPLG | | | | | | |
| | | CCTTAGGCTACTACTACATGGACGTCTGGGGCAAAGGGACC | | | | | | | YYYMDVWGKGTTVT | | | | | | |
| | | ACGGTCACCGTCTCCTCA | | | | | | | VSS | | | | | | |
| ADI-81901 | 1286 | GAGGTGCAGCTGGTGGAGTCTGGGGGAGGCTTGGTCCAGCC | | | | | | 6938 | EVQLVESGGGLVQP | | 7290 | 11 | | 10 | |
| | | TGGGGGGTCCCTGAGACTCTCCTGTTCAGCCTCTGGATTCA | | | | | | | GGSLRLSCSASGFS | | | | | | |
| | | GCTTCAGTACCTATTCTATGCACTGGGTCCGCCAGGCTCCA | | | | | | | FSTYSMHWVRQAPG | | | | | | |
| | | GGGAAGGGACTGGAATATGTTTCAGATATAAGTGGTGATGG | | | | | | | KGLEYVSDISGDGL | | | | | | |
| | | CCTTATCACATACTACGCAGACTCCGTGAAGGGCAGATTCA | | | | | | | ITYYADSVKGRFTI | | | | | | |
| | | CCATCACCAGAGACAATTCCAAGAATACGCTCTATCTTCAC | | | | | | | TRDNSKNTLYLHMS | | | | | | |
| | | ATGAGCAGTCTGAGAGCTGAGGACACGGCTGTCTATTACTG | | | | | | | SLRAEDTAVYYCVR | | | | | | |
| | | TGTGAGAGATGTGAGTGTGGATACATCTATGGTTACAGTAT | | | | | | | DVSVDTSMVTVFEY | | | | | | |
| | | TTGAATACTGGGGCCAGGGAACCCTGGTCACCGTCTCCTCA | | | | | | | WGQGTLVTVSS | | | | | | |
| ADI-81532 | 1291 | TGGGGGGTCCCTGAGACTCTCCTGTGCAGCCTCTGGATTCA | | | | | | 6939 | QVQLVQSGGGLAQP | | 7291 | 10 | | 8 | |
| | | TCTTCAGTAGTTACGACATGCACTGGGTCCGCCAAGTCACA | | | | | | | GGSLRLSCAASGFI | | | | | | |
| | | GGAAAAGGTCTGGAATGGGTCGCAGCTATTGGTACTGCTGG | | | | | | | FSSYDMHWVRQVTG | | | | | | |
| | | TGACACATACTATGAAGACTCCGTGAAGGGCCGGTTCACCA | | | | | | | KGLEWVAAIGTAGD | | | | | | |
| | | TCTCCAGAGAGAATGCCAAGAACTCCGTGTATCTTCAAATG | | | | | | | TYYEDSVKGRFTIS | | | | | | |
| | | AACAGCCTGAGAGCCGGGGACACGGCTATGTATTACTGTGC | | | | | | | RENAKNSVYLQMNS | | | | | | |
| | | AAGAGGTGATCAACCTATTACGATTTCTGGAGTGGTTGAAA | | | | | | | LRAGDTAMYYCARG | | | | | | |
| | | CCTACTACATGGACGTGTGGGGCAAAGGGACCACGGTCACC | | | | | | | DQPITISGVVETYY | | | | | | |
| | | GTCTCCTCA | | | | | | | MDVWGKGTTVTVSS | | | | | | |
| ADI-81537 | 1291 | TGGGGGGTCCCTGAGACTCTCCTGTGCAGCCTCTGGATTCA | | | | | | 6940 | QVQLVQSGGGLAQP | | 7291 | 10 | | 8 | |
| | | TCTTCAGTAGTTACGACATGCACTGGGTCCGCCAAGTCACA | | | | | | | GGSLRLSCAASGFI | | | | | | |
| | | GGAAAAGGTCTGGAATGGGTCGCAGCTATTGGTACTGCTGG | | | | | | | FSSYDMHWVRQVTG | | | | | | |
| | | TGACACATACTATGAAGACTCCGTGAAGGGCCGGTTCACCA | | | | | | | KGLEWVAAIGTAGD | | | | | | |
| | | TCTCCAGAGAGAATGCCAAGAACTCCGTGTATCTTCAAATG | | | | | | | TYYEDSVKGRFTIS | | | | | | |
| | | AACAGCCTGAGAGCCGGGGACACGGCTATGTATTACTGTGC | | | | | | | RENAKNSVYLQMNS | | | | | | |
| | | AAGAGGTGATCAACCTATTACGATTTCTGGAGTGGTTGAAA | | | | | | | LRAGDTAMYYCARG | | | | | | |
| | | CCTACTACATGGACGTGTGGGGCAAAGGGACCACGGTCACC | | | | | | | DQPITISGVVETYY | | | | | | |
| | | GTCTCCTCA | | | | | | | MDVWGKGTTVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81565 | 27 | IGHV1- | QVQLV | 50 | GTFSSQ | 5598 | WVRQAP | 433 | KIFPIL | 5963 | RVTITA | 6274 | ARESGYS | 6579 | WGRGTL |
| | | 69 | QSGAE | | TIS | | GQGLEW | | GVTNYA | | DKSTST | | ASGSVIY | | VTVSS |
| | | | VKKPG | | | | MG | | ERFQG | | AYMELS | | FDL | | |
| | | | SSVKV | | | | | | | | GLRSED | | | | |
| | | | SCKAS | | | | | | | | TAEYYC | | | | |
| | | | G | | | | | | | | | | | | |
| ADI-81811 | 85 | IGHV3- | EVQLL | 5392 | FNFRSY | 5599 | WVRQAP | 431 | AISHDG | 5964 | RFTISR | 6275 | AKDAVGG | 6580 | WGQGTL |
| | | 30 | ESGGG | | GMH | | GKGLEW | | TNPYYA | | DNSKDT | | YCRGGSC | | VTVSS |
| | | | VVQPG | | | | VA | | DSVKG | | LYLQMN | | YDFDYFD | | |
| | | | RSLRL | | | | | | | | SLRAED | | Y | | |
| | | | SCAVS | | | | | | | | TAVYHC | | | | |
| | | | G | | | | | | | | | | | | |
| ADI-81883 | 59 | IGHV3- | EVQLL | 5393 | FTFSTY | 228 | WVRQAP | 443 | DISGDG | 5961 | RFTITR | 6276 | VRDVSVD | 6577 | WGQGTL |
| | | 64D | ESGGG | | SMH | | GKGLEY | | LITYYA | | DNSKNT | | TSMVTVF | | VTVSS |
| | | | LVQPG | | | | VS | | DSVKG | | LYLQMS | | EY | | |
| | | | GSLRL | | | | | | | | SLRAED | | | | |
| | | | SCSAS | | | | | | | | TAVYYC | | | | |
| | | | G | | | | | | | | | | | | |
| ADI-81686 | 56 | IGHV4- | QVQLQ | 5394 | GSFSGK | 5600 | WIRQPA | 5814 | EINDSG | 5965 | RVTISV | 6277 | ARERVIL | 6581 | WGKGTT |
| | | 34 | QWGTG | | YWS | | GKGLEW | | TTNYNP | | DTSKNQ | | LSGTSLH | | VTVSS |
| | | | LLKPS | | | | IG | | SLKS | | FSLKLR | | YNYYMDV | | |
| | | | ETLSL | | | | | | | | SVTAAD | | | | |
| | | | TCAVY | | | | | | | | TADYYC | | | | |
| | | | G | | | | | | | | | | | | |
| ADI-81825 | 5 | IGHV3- | EVQLL | 5395 | FTFSNA | 5601 | WVRQAP | 432 | RIKSNF | 5966 | RFSISR | 6278 | TTDVVDD | 6582 | WGQGTR |
| | | 15 | ESGGG | | WMN | | GKGLEW | | DGGTTD | | DDSKNT | | DFRSGYI | | VTVSS |
| | | | LVKPG | | | | VG | | YAAPVK | | LYLQMN | | EHDAFDF | | |
| | | | GSLRL | | | | | | G | | SLNTED | | | | |
| | | | SCAAT | | | | | | | | TAVYYC | | | | |
| | | | G | | | | | | | | | | | | |
| ADI-81748 | 24 | IGHV4- | QVQLL | 5396 | VSISDY | 5602 | WIRQPP | 445 | YISYSG | 5967 | RVTISV | 6279 | ARELPTG | 6583 | WGQGTL |
| | | 59 | ESGPG | | YWS | | GKGLEW | | STDYNP | | DTSKNQ | | NSYEYYF | | VTVSS |
| | | | LVKPS | | | | IG | | SLKS | | VSLKLT | | DY | | |
| | | | ETLSL | | | | | | | | SVTAAD | | | | |
| | | | TCTVS | | | | | | | | TAVYYC | | | | |
| | | | G | | | | | | | | | | | | |
| ADI-81840 | 18 | IGHV3- | QVQLL | 74 | FTFSSY | 5603 | WVRQAP | 437 | SVSGSG | 5968 | RFTISR | 6280 | GKAIIGS | 6584 | WGQGTL |
| | | 23 | ESGGG | | AMS | | GKGLEW | | ANTYYA | | DNSKNT | | NGVGEY | | VTVSS |
| | | | LVQPG | | | | VS | | DSVKG | | VSLQMN | | | | |
| | | | GSLRL | | | | | | | | SLRAED | | | | |
| | | | SCAAS | | | | | | | | AAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81565 | 1288 | TGGGTCCTCGGTGAAGGTCTCCTGCAAGGCTTCTGGAGGCA | | | | | | 6941 | QVQLVQSGAEVKKP | | 7292 | 10 | | 9 | |
| | | CCTTCAGCAGTCAAACAATCAGTTGGGTGCGACAGGCCCCT | | | | | | | GSSVKVSCKASGGT | | | | | | |
| | | GGACAAGGGCTTGAGTGGATGGGAAAGATCTTCCCTATCCT | | | | | | | FSSQTISWVRQAPG | | | | | | |
| | | TGGTGTAACAAACTACGCAGAGAGGTTTCAGGGCAGAGTCA | | | | | | | QGLEWMGKIFPILG | | | | | | |
| | | CGATTACCGCGGACAAGTCGACCAGCACAGCCTACATGGAG | | | | | | | VTNYAERFQGRVTI | | | | | | |
| | | CTGAGCGGCCTGAGATCTGAGGACACGGCCGAGTATTACTG | | | | | | | TADKSTSTAYMELS | | | | | | |
| | | TGCGAGAGAGTCGGGTTACTCTGCTTCGGGGAGTGTTATCT | | | | | | | GLRSEDTAEYYCAR | | | | | | |
| | | ACTTCGATCTCTGGGGCCGTGGCACCCTGGTCACCGTCTCC | | | | | | | ESGYSASGSVIYFD | | | | | | |
| | | TCA | | | | | | | LWGRGTLVTVSS | | | | | | |
| ADI-81811 | 1286 | GAGGTGCAGCTGTTGGAGTCTGGGGGAGGCGTGGTCCAGCC | | | | | | 6942 | EVQLLESGGGVVQP | | 7293 | 10 | | 9 | |
| | | TGGGAGGTCCCTGAGACTCTCCTGTGCAGTCTCTGGATTCA | | | | | | | GRSLRLSCAVSGFN | | | | | | |
| | | ACTTCAGAAGTTATGGCATGCACTGGGTCCGCCAGGCTCCA | | | | | | | FRSYGMHWVRQAPG | | | | | | |
| | | GGCAAGGGGCTGGAGTGGGTGGCAGCTATATCACATGATGG | | | | | | | KGLEWVAAISHDGT | | | | | | |
| | | AACTAATCCATATTATGCAGACTCCGTGAAGGGGCGATTCA | | | | | | | NPYYADSVKGRFTI | | | | | | |
| | | CCATCTCCAGAGACAATTCCAAGGACACGCTGTATCTGCAA | | | | | | | SRDNSKDTLYLQMN | | | | | | |
| | | ATGAACAGCCTGAGAGCGGAGGACACGGCTGTGTATCACTG | | | | | | | SLRAEDTAVYHCAK | | | | | | |
| | | TGCGAAAGATGCCGTTGGGGGATATTGTAGGGGTGGTAGTT | | | | | | | DAVGGYCRGGSCYD | | | | | | |
| | | GTTATGACTTTGACTACTTTGACTACTGGGGCCAGGGAACC | | | | | | | FDYFDYWGQGTLVT | | | | | | |
| | | CTGGTCACCGTCTCCTCA | | | | | | | VSS | | | | | | |
| ADI-81883 | 1286 | GAGGTGCAGCTGTTGGAGTCTGGGGGAGGCTTGGTCCAGCC | | | | | | 6943 | EVQLLESGGGLVQP | | 7294 | 9 | | 8 | |
| | | TGGGGGATCCCTGAGACTCTCCTGTTCAGCCTCTGGATTCA | | | | | | | GGSLRLSCSASGFT | | | | | | |
| | | CCTTCAGTACCTATTCTATGCACTGGGTCCGCCAGGCTCCA | | | | | | | FSTYSMHWVRQAPG | | | | | | |
| | | GGGAAGGGACTGGAATATGTTTCAGATATAAGTGGTGATGG | | | | | | | KGLEYVSDISGDGL | | | | | | |
| | | GCTTATCACATACTACGCAGACTCCGTGAAGGGCAGATTCA | | | | | | | ITYYADSVKGRFTI | | | | | | |
| | | CCATCACCAGAGACAATTCCAAGAATACGCTCTATCTTCAA | | | | | | | TRDNSKNTLYLQMS | | | | | | |
| | | ATGAGCAGTCTGAGAGCTGAGGACACGGCTGTCTATTACTG | | | | | | | SLRAEDTAVYYCVR | | | | | | |
| | | TGTGAGAGATGTGAGTGTGGATACATCTATGGTTACAGTAT | | | | | | | DVSVDTSMVTVFEY | | | | | | |
| | | TTGAATACTGGGGCCAGGGAACCCTGGTCACCGTCTCCTCA | | | | | | | WGQGTLVTVSS | | | | | | |
| ADI-81686 | 1291 | CAGGTGCAGCTACAGCAGTGGGGCACAGGACTGTTGAAGCC | | | | | | 6944 | QVQLQQWGTGLLKP | | 7295 | 8 | | 6 | |
| | | TTCGGAGACCCTGTCCCTCACCTGCGCTGTCTATGGTGGGT | | | | | | | SETLSLTCAVYGGS | | | | | | |
| | | CCTTCAGTGGTAAGTACTGGAGCTGGATCCGCCAGCCCGCA | | | | | | | FSGKYWSWIRQPAG | | | | | | |
| | | GGGAAGGGGCTGGAATGGATTGGAGAAATCAATGATAGTGG | | | | | | | KGLEWIGEINDSGT | | | | | | |
| | | AACCACCAACTACAACCCGTCCCTCAAGAGTCGAGTCACCA | | | | | | | TNYNPSLKSRVTIS | | | | | | |
| | | TATCAGTAGACACGTCCAAGAACCAGTTCTCCCTGAAACTG | | | | | | | VDTSKNQFSLKLRS | | | | | | |
| | | AGGTCTGTGACCGCCGCGGACACGGCTGACTATTATTGTGC | | | | | | | VTAADTADYYCARE | | | | | | |
| | | GAGAGAACGGGTTATCCTACTAAGTGGAACGTCTCTCCACT | | | | | | | RVILLSGTSLHYNY | | | | | | |
| | | ACAACTACTACATGGACGTCTGGGGCAAAGGGACCACGGTC | | | | | | | YMDVWGKGTTVTVS | | | | | | |
| | | ACCGTCTCCTCA | | | | | | | S | | | | | | |
| ADI-81825 | 6812 | GAGGTGCAGCTGTTGGAGTCTGGGGGAGGCCTGGTAAAGCC | | | | | | 6945 | EVQLLESGGGLVKP | | 7296 | 8 | | 6 | |
| | | TGGGGGGTCCCTCAGACTCTCCTGTGCAGCCACTGGATTCA | | | | | | | GGSLRLSCAATGFT | | | | | | |
| | | CTTTCAGTAATGCCTGGATGAATTGGGTCCGCCAGGCTCCA | | | | | | | FSNAWMNWVRQAPG | | | | | | |
| | | GGGAAGGGGCTGGAGTGGGTTGGCCGTATTAAAAGCAATTT | | | | | | | KGLEWVGRIKSNFD | | | | | | |
| | | TGACGGTGGGACAACAGACTACGCTGCACCCGTGAAAGGCA | | | | | | | GGTTDYAAPVKGRF | | | | | | |
| | | GATTCAGCATCTCAAGAGATGATTCAAAAAACACGCTGTAT | | | | | | | SISRDDSKNTLYLQ | | | | | | |
| | | CTGCAAATGAACAGTCTGAACACCGAGGACACAGCCGTCTA | | | | | | | MNSLNTEDTAVYYC | | | | | | |
| | | TTATTGTACCACAGATGTAGTGGATGACGATTTTCGGAGTG | | | | | | | TTDVVDDDFRSGYI | | | | | | |
| | | GCTACATAGAGCATGATGCTTTTGATTTCTGGGGCCAAGGG | | | | | | | EHDAFDFWGQGTRV | | | | | | |
| | | ACAAGGGTCACCGTCTCCTCA | | | | | | | TVSS | | | | | | |
| ADI-81748 | 1286 | CAGGTGCAGCTGTTGGAGTCGGGCCCAGGACTGGTGAAGCC | | | | | | 6946 | QVQLLESGPGLVKP | | 7297 | 7 | | 6 | |
| | | TTCGGAGACACTGTCCCTCACCTGCACTGTCTCAGGTGTCT | | | | | | | SETLSLTCTVSGVS | | | | | | |
| | | CCATCAGTGATTACTACTGGAGCTGGATCCGACAGCCCCCA | | | | | | | ISDYYWSWIRQPPG | | | | | | |
| | | GGGAAGGGACTGGAGTGGATTGGGTATATCTCTTACAGTGG | | | | | | | KGLEWIGYISYSGS | | | | | | |
| | | GAGCACCGACTACAACCCCTCCCTCAAGAGTCGAGTCACCA | | | | | | | TDYNPSLKSRVTIS | | | | | | |
| | | TATCAGTGGACACGTCCAAGAACCAGGTCTCCCTGAAACTG | | | | | | | VDTSKNQVSLKLTS | | | | | | |
| | | ACCTCTGTGACCGCTGCGGACACGGCCGTGTATTACTGTGC | | | | | | | VTAADTAVYYCARE | | | | | | |
| | | GAGAGAATTGCCTACTGGAAATAGTTATGAGTACTACTTTG | | | | | | | LPTGNSYEYYFDYW | | | | | | |
| | | ACTACTGGGGCCAGGGAACCCTGGTCACCGTCTCCTCA | | | | | | | GQGTLVTVSS | | | | | | |
| ADI-81840 | 1286 | CAGGTGCAGCTGTTGGAGTCTGGGGGAGGCTTGGTACAGCC | | | | | | 6947 | QVQLLESGGGLVQP | | 7298 | 7 | | 7 | |
| | | GGGGGGGTCCCTGAGACTCTCCTGTGCAGCCTCTGGATTCA | | | | | | | GGSLRLSCAASGFT | | | | | | |
| | | CCTTTAGCAGTTATGCCATGAGCTGGGTCCGCCAGGCTCCA | | | | | | | FSSYAMSWVRQAPG | | | | | | |
| | | GGGAAGGGGCTGGAGTGGGTCTCTTCTGTTAGTGGTAGTGG | | | | | | | KGLEWVSSVSGSGA | | | | | | |
| | | TGCTAACACATATTACGCAGACTCCGTGAAGGGCCGGTTCA | | | | | | | NTYYADSVKGRFTI | | | | | | |
| | | CCATCTCCAGAGACAATTCCAAGAACACGGTGTCTCTGCAA | | | | | | | SRDNSKNTVSLQMN | | | | | | |
| | | ATGAACAGCCTGAGAGCCGAGGACGCGGCCGTATATTACTG | | | | | | | SLRAEDAAVYYCGK | | | | | | |
| | | TGGGAAAGCCATAATCGGAAGTAACGGGGTGGGAGAGTACT | | | | | | | AIIGSNGVGEYWGQ | | | | | | |
| | | GGGGCCAGGGAACCCTGGTCACCGTCTCCTCA | | | | | | | GTLVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81692 | 62 | IGHV5- | EVQLV | 5397 | YSFTNY | 5604 | WVRQMP | 476 | VIYPGD | 5969 | QVTFSA | 6281 | ARQTGIV | 6585 | WGKGTT |
| | | 51 | QSGAE | | WIG | | GKGLEW | | SDTRYS | | DKSIST | | GASRYQY | | VTVSS |
| | | | VKKPG | | | | MG | | PSFQG | | AYLQWS | | YYYMDV | | |
| | | | ESLKI | | | | | | | | SLKASD | | | | |
| | | | SCKGS | | | | | | | | TAIYYC | | | | |
| | | | G | | | | | | | | | | | | |
| ADI-81730 | 7 | IGHV3- | EVQLL | 5355 | FTFSSY | 307 | WVRQAP | 437 | YISSSG | 5970 | RFTISR | 854 | ARAPRTN | 6586 | WGQGTL |
| | | 48 | ESGGG | | EMN | | GKGLEW | | STIYYA | | DNAKNS | | WNDVWGT | | VTVSS |
| | | | LVQPG | | | | VS | | DSVKG | | LYLQMN | | FDY | | |
| | | | GSLRL | | | | | | | | SLRAED | | | | |
| | | | SCAAS | | | | | | | | TAVYYC | | | | |
| | | | G | | | | | | | | | | | | |
| ADI-81889 | 65 | IGHV1- | QVQLV | 5398 | NTFTNN | 5605 | WVRQVP | 5815 | IINPVG | 5971 | RVTLTS | 6282 | ARGRIPP | 6587 | WGQGTL |
| | | 46 | QSGAE | | YIQ | | GQGVEW | | GSRTYA | | DTSTRT | | DSDGYFD | | VTVSS |
| | | | VKKHG | | | | MG | | QEFQG | | LYMEMS | | F | | |
| | | | ASVKV | | | | | | | | SLTSED | | | | |
| | | | SCKAS | | | | | | | | TAIYFC | | | | |
| | | | G | | | | | | | | | | | | |
| ADI-81608 | 32 | IGHV4- | QVQLV | 5399 | GSIRSV | 5606 | WIRQHP | 450 | YIYHNG | 5972 | RVTMSL | 6283 | ARLGPSF | 6588 | WDQGTP |
| | | 31 | QSGPG | | YYYWT | | GKGLEW | | TTYSNP | | DTSKNQ | | YDRSGSS | | VTVSS |
| | | | LVKPS | | | | IG | | SLRS | | FSLKVR | | RLNYFDS | | |
| | | | QTLSL | | | | | | | | SVTAAD | | | | |
| | | | TCTVS | | | | | | | | TAVYFC | | | | |
| | | | G | | | | | | | | | | | | |
| ADI-81616 | 73 | IGHV3- | RCTLV | 5400 | FTFSSY | 5607 | WVRQAP | 437 | SISGSG | 5973 | RFTISR | 6284 | ARFGGSD | 6589 | WGQGTL |
| | | 23 | QSGGD | | VMT | | GKGLEW | | GARYSA | | DNSNNT | | YRGGWSS | | VTVSS |
| | | | LVQPG | | | | VS | | DSLEG | | LYLEMT | | Y | | |
| | | | GSLRL | | | | | | | | SLRDED | | | | |
| | | | SCEAS | | | | | | | | TAVYYC | | | | |
| | | | G | | | | | | | | | | | | |
| ADI-81734 | 11 | IGHV3- | EVQLV | 5401 | FTFTSD | 5608 | WVRQVI | 5816 | GIGTSG | 5974 | RFIISR | 6285 | ARGVAAG | 6590 | WGKGTT |
| | | 13 | ESGGG | | DMH | | GKGLEW | | DRYYAD | | EDARNS | | NYNLYYY | | VTVSS |
| | | | LVQSG | | | | VS | | SVKG | | LYLQMN | | MDV | | |
| | | | GSLRL | | | | | | | | SLRAGD | | | | |
| | | | SCAAS | | | | | | | | TAIYYC | | | | |
| | | | G | | | | | | | | | | | | |
| ADI-81767 | 43 | IGHV1- | EVQLV | 5402 | GPFSKY | 5609 | WVRQAP | 5817 | RIILVL | 5975 | RVTMTA | 6286 | ATKDAPT | 6591 | WGQGTL |
| | | 69 | ESGAE | | AMN | | GQRPEW | | GITNYA | | DKSTNT | | VNSNGGL | | VTVSS |
| | | | VKKPG | | | | LG | | QKFQG | | AYMELG | | DF | | |
| | | | SSVKV | | | | | | | | SLTSDD | | | | |
| | | | SCKAS | | | | | | | | TAVYYC | | | | |
| | | | G | | | | | | | | | | | | |
| ADI-81688 | 58 | IGHV1- | EVQLL | 5403 | YSFTTY | 5610 | WVRQAP | 5818 | LITPSD | 5976 | RVTMTR | 6287 | ARGDWNK | 6592 | WGKGTT |
| | | 46 | ESGDE | | YIH | | GPGLEW | | GGASYA | | DTSTST | | ISYYYMD | | VTVSS |
| | | | VKKPW | | | | MG | | QKFQG | | VYMEMS | | V | | |
| | | | ASVTV | | | | | | | | SLTIED | | | | |
| | | | SCKAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81692 | 1291 | GAGGTGCAGCTGGTGCAGTCTGGAGCAGAGGTGAAGAAGCC | | | | | | 6948 | EVQLVQSGAEVKKP | | 7299 | 4 | | 4 | |
| | | CGGGGAGTCTCTGAAGATCTCCTGTAAGGGTTCTGGATACA | | | | | | | GESLKISCKGSGYS | | | | | | |
| | | GCTTTACCAACTACTGGATCGGCTGGGTGCGCCAGATGCCC | | | | | | | FTNYWIGWVRQMPG | | | | | | |
| | | GGGAAAGGCCTGGAGTGGATGGGTGTCATCTATCCTGGTGA | | | | | | | KGLEWMGVIYPGDS | | | | | | |
| | | CTCTGATACCAGATATAGCCCGTCCTTCCAAGGTCAGGTCA | | | | | | | DTRYSPSFQGQVTF | | | | | | |
| | | CCTTCTCAGCCGACAAGTCCATCAGCACCGCCTACCTGCAG | | | | | | | SADKSISTAYLQWS | | | | | | |
| | | TGGAGCAGCCTGAAGGCCTCGGACACCGCCATATATTATTG | | | | | | | SLKASDTAIYYCAR | | | | | | |
| | | TGCGAGACAGACAGGTATAGTGGGAGCCTCGCGATACCAGT | | | | | | | QTGIVGASRYQYYY | | | | | | |
| | | ACTACTACTACATGGACGTCTGGGGCAAAGGGACCACGGTC | | | | | | | YMDVWGKGTTVTVS | | | | | | |
| | | ACCGTCTCCTCA | | | | | | | S | | | | | | |
| ADI-81730 | 1286 | TGGAGGGTCCCTGAGACTCTCCTGTGCAGCCTCTGGATTCA | | | | | | 6949 | EVQLLESGGGLVQP | | 7300 | 0 | | 0 | |
| | | CCTTCAGTAGTTATGAAATGAACTGGGTCCGCCAGGCTCCA | | | | | | | GGSLRLSCAASGFT | | | | | | |
| | | GGGAAGGGGCTGGAGTGGGTTTCATACATTAGTAGTAGTGG | | | | | | | FSSYEMNWVRQAPG | | | | | | |
| | | TAGTACCATATACTACGCAGACTCTGTGAAGGGCCGATTCA | | | | | | | KGLEWVSYISSSGS | | | | | | |
| | | CCATCTCCAGAGACAACGCCAAGAACTCACTGTATCTGCAA | | | | | | | TIYYADSVKGRFTI | | | | | | |
| | | ATGAACAGCCTGAGAGCCGAGGACACGGCTGTTTATTACTG | | | | | | | SRDNAKNSLYLQMN | | | | | | |
| | | TGCGAGAGCCCCACGTACCAACTGGAACGACGTTTGGGGGA | | | | | | | SLRAEDTAVYYCAR | | | | | | |
| | | CATTTGACTACTGGGGCCAGGGAACCCTGGTCACCGTCTCC | | | | | | | APRTNWNDVWGTFD | | | | | | |
| | | TCA | | | | | | | YWGQGTLVTVSS | | | | | | |
| ADI-81889 | 1286 | CAGGTGCAGCTGGTGCAGTCTGGGGCTGAGGTGAAGAAGCA | | | | | | 6950 | QVQLVQSGAEVKKH | | 7301 | 25 | | 20 | |
| | | TGGGGCCTCAGTCAAGGTTTCCTGCAAGGCATCTGGAAACA | | | | | | | GASVKVSCKASGNT | | | | | | |
| | | CCTTCACCAACAACTATATACAGTGGGTGCGACAGGTCCCT | | | | | | | FTNNYIQWVRQVPG | | | | | | |
| | | GGACAAGGGGTTGAGTGGATGGGAATAATCAACCCTGTTGG | | | | | | | QGVEWMGIINPVGG | | | | | | |
| | | TGGTAGTAGAACTTACGCACAGGAGTTCCAGGGCAGAGTCA | | | | | | | SRTYAQEFQGRVTL | | | | | | |
| | | CCTTGACCAGTGACACGTCCACGAGAACATTGTACATGGAG | | | | | | | TSDTSTRTLYMEMS | | | | | | |
| | | ATGAGCAGCCTGACATCTGAGGACACGGCCATATATTTTTG | | | | | | | SLTSEDTAIYFCAR | | | | | | |
| | | TGCGAGAGGTCGAATCCCCCCCGATAGTGATGGTTATTTTG | | | | | | | GRIPPDSDGYFDFW | | | | | | |
| | | ACTTCTGGGGCCAGGGAACCCTGGTCACGGTCTCCTCA | | | | | | | GQGTLVTVSS | | | | | | |
| ADI-81608 | 6813 | CAGGTGCAGCTGGTGCAGTCTGGCCCAGGACTGGTGAAGCC | | | | | | 6951 | QVQLVQSGPGLVKP | | 7302 | 18 | | 15 | |
| | | TTCACAGACCCTGTCCCTCACCTGCACTGTCTCTGGTGGCT | | | | | | | SQTLSLTCTVSGGS | | | | | | |
| | | CCATCAGAAGTGTTTACTACTACTGGACCTGGATCCGCCAG | | | | | | | IRSVYYYWTWIRQH | | | | | | |
| | | CACCCAGGGAAGGGCCTAGAGTGGATTGGGTACATCTATCA | | | | | | | PGKGLEWIGYIYHN | | | | | | |
| | | CAATGGGACCACCTACTCCAATCCGTCCCTCAGGAGCCGAG | | | | | | | GTTYSNPSLRSRVT | | | | | | |
| | | TTACCATGTCACTGGACACGTCTAAGAACCAGTTTTCCCTG | | | | | | | MSLDTSKNQFSLKV | | | | | | |
| | | AAGGTGAGGTCTGTGACTGCCGCGGACACGGCCGTGTATTT | | | | | | | RSVTAADTAVYFCA | | | | | | |
| | | CTGTGCGAGACTTGGACCGTCTTTCTATGATAGGAGTGGTT | | | | | | | RLGPSFYDRSGSSR | | | | | | |
| | | CCTCACGTCTCAACTACTTTGACTCGTGGGACCAGGGGACC | | | | | | | LNYFDSWDQGTPVT | | | | | | |
| | | CCGGTCACCGTCTCCTCA | | | | | | | VSS | | | | | | |
| ADI-81616 | 1286 | AGGTGCACGCTGGTGCAATCTGGGGGAGACTTGGTGCAGCC | | | | | | 6952 | RCTLVQSGGDLVQP | | 7303 | 18 | | 14 | |
| | | GGGGGGGTCCCTGAGACTCTCCTGTGAAGCCTCTGGATTCA | | | | | | | GGSLRLSCEASGFT | | | | | | |
| | | CATTTAGCAGCTATGTCATGACGTGGGTCCGCCAGGCTCCA | | | | | | | FSSYVMTWVRQAPG | | | | | | |
| | | GGGAAGGGGCTGGAGTGGGTCTCAAGTATCAGTGGTAGTGG | | | | | | | KGLEWVSSISGSGG | | | | | | |
| | | TGGTGCCAGATACTCCGCAGACTCCTTGGAGGGCCGGTTCA | | | | | | | ARYSADSLEGRFTI | | | | | | |
| | | CCATCTCCAGAGACAACTCGAACAACACTCTGTATTTGGAA | | | | | | | SRDNSNNTLYLEMT | | | | | | |
| | | ATGACTAGCCTGAGAGACGAGGACACGGCCGTATACTACTG | | | | | | | SLRDEDTAVYYCAR | | | | | | |
| | | TGCCAGATTTGGAGGGTCCGATTACCGCGGTGGCTGGTCTA | | | | | | | FGGSDYRGGWSSYW | | | | | | |
| | | GTTATTGGGGCCAGGGAACCCTGGTCACTGTCTCCTCA | | | | | | | GQGTLVTVSS | | | | | | |
| ADI-81734 | 1291 | GAGGTGCAGCTGGTGGAGTCTGGGGGAGGCTTGGTTCAGTC | | | | | | 6953 | EVQLVESGGGLVQS | | 7304 | 18 | | 14 | |
| | | GGGGGGGTCCCTGAGACTCTCCTGTGCAGCCTCTGGATTCA | | | | | | | GGSLRLSCAASGFT | | | | | | |
| | | CCTTCACTAGCGACGACATGCACTGGGTCCGCCAAGTAATA | | | | | | | FTSDDMHWVRQVIG | | | | | | |
| | | GGAAAAGGTCTGGAGTGGGTCTCGGGGATTGGCACTTCTGG | | | | | | | KGLEWVSGIGTSGD | | | | | | |
| | | TGATAGATACTATGCAGACTCCGTGAAGGGCCGCTTCATCA | | | | | | | RYYADSVKGRFIIS | | | | | | |
| | | TCTCTAGAGAAGATGCCAGGAACTCCTTGTATCTTCAAATG | | | | | | | REDARNSLYLQMNS | | | | | | |
| | | AACAGCCTGAGAGCCGGGGACACGGCTATATATTATTGTGC | | | | | | | LRAGDTAIYYCARG | | | | | | |
| | | AAGAGGGGTAGCTGCCGGCAATTATAACCTCTACTACTACA | | | | | | | VAAGNYNLYYYMDV | | | | | | |
| | | TGGACGTCTGGGGCAAAGGGACCACGGTCACCGTCTCCTCA | | | | | | | WGKGTTVTVSS | | | | | | |
| ADI-81767 | 1286 | GAGGTGCAGCTGGTGGAGTCTGGGGCTGAGGTGAAGAAGCC | | | | | | 6954 | EVQLVESGAEVKKP | | 7305 | 18 | | 15 | |
| | | TGGGTCTTCGGTGAAGGTCTCCTGCAAGGCTTCTGGAGGCC | | | | | | | GSSVKVSCKASGGP | | | | | | |
| | | CCTTCAGTAAATATGCTATGAACTGGGTGCGACAGGCCCCT | | | | | | | FSKYAMNWVRQAPG | | | | | | |
| | | GGACAACGACCTGAGTGGCTGGGAAGGATCATTCTTGTCCT | | | | | | | QRPEWLGRIILVLG | | | | | | |
| | | TGGTATCACAAACTACGCACAGAAGTTCCAGGGCAGAGTCA | | | | | | | ITNYAQKFQGRVTM | | | | | | |
| | | CAATGACCGCGGACAAATCCACGAATACAGCCTACATGGAG | | | | | | | TADKSTNTAYMELG | | | | | | |
| | | CTGGGCAGCCTGACATCTGACGACACGGCCGTGTATTACTG | | | | | | | SLTSDDTAVYYCAT | | | | | | |
| | | TGCGACCAAGGATGCCCCCACTGTAAATAGTAACGGTGGTC | | | | | | | KDAPTVNSNGGLDF | | | | | | |
| | | TGGACTTCTGGGGCCAGGGAACCCTGGTCACCGTCTCCTCA | | | | | | | WGQGTLVTVSS | | | | | | |
| ADI-81688 | 1291 | GAGGTGCAGCTGCTGGAGTCTGGGGATGAGGTGAAGAAGCC | | | | | | 6955 | EVQLLESGDEVKKP | | 7306 | 16 | | 14 | |
| | | GTGGGCCTCAGTGACGGTTTCCTGCAAGGCATCTGGATACA | | | | | | | WASVTVSCKASGYS | | | | | | |
| | | GCTTCACCACTTACTATATACACTGGGTGCGACAGGCCCCT | | | | | | | FTTYYIHWVRQAPG | | | | | | |
| | | GGACCAGGGCTTGAATGGATGGGACTAATCACCCCTAGTGA | | | | | | | PGLEWMGLITPSDG | | | | | | |
| | | TGGCGGCGCAAGCTACGCACAGAAGTTCCAGGGCAGAGTCA | | | | | | | GASYAQKFQGRVTM | | | | | | |
| | | CCATGACCAGAGACACGTCCACGAGTACAGTCTACATGGAG | | | | | | | TRDTSTSTVYMEMS | | | | | | |
| | | ATGAGCAGCCTGACAATTGAGGACACGGCCGTATATTATTG | | | | | | | SLTIEDTAVYYCAR | | | | | | |
| | | TGCGAGAGGGGACTGGAACAAAATAAGCTACTACTACATGG | | | | | | | GDWNKISYYYMDVW | | | | | | |
| | | ACGTCTGGGGCAAAGGGACCACGGTCACCGTCTCCTCA | | | | | | | GKGTTVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81697 | 67 | IGHV4- | RGAAA | 5404 | ASISSS | 5611 | WVRQPP | 444 | EIYHVG | 5977 | RVTISI | 6288 | ARVVLSD | 6593 | WGQGTL |
| | | 4 | GVGPR | | NWWS | | GKGLEW | | STNYNP | | DKSNNQ | | FKEAAS | | VTVSS |
| | | | TGEAS | | | | IG | | SLKS | | FSLKLN | | | | |
| | | | GTLSL | | | | | | | | SVTAAD | | | | |
| | | | TCAVS | | | | | | | | TAVYYC | | | | |
| | | | G | | | | | | | | | | | | |
| ADI-81549 | 16 | IGHV3- | QVQLV | 5405 | FSFSLF | 5612 | WVRQAP | 437 | SITGSA | 5978 | RFTISR | 6289 | AKGMDGA | 6594 | WGQGTL |
| | | 23 | QSGGG | | AMN | | GKGLEW | | DTTYYA | | DNSKNT | | PGFYFDY | | VTVSS |
| | | | LVQPG | | | | VS | | DSVKG | | LYLQMN | | | | |
| | | | GSLRL | | | | | | | | SLSAED | | | | |
| | | | SCTAS | | | | | | | | TAVYYC | | | | |
| | | | G | | | | | | | | | | | | |
| ADI-81562 | 28 | IGHV3- | QVQLV | 24 | LTVSSN | 5613 | WLRQAP | 5819 | VMFSGG | 5979 | RFTISR | 6290 | ARDLPPY | 6595 | WGQGTT |
| | | 53 | ESGGG | | YMT | | GKGLEW | | STFYAD | | HNSNNT | | GMDV | | VTVSS |
| | | | LVQPG | | | | VS | | SVKG | | LYLQMS | | | | |
| | | | GSLRL | | | | | | | | SLGPED | | | | |
| | | | SCAAS | | | | | | | | TATYYC | | | | |
| | | | G | | | | | | | | | | | | |
| ADI-81813 | 87 | IGHV3- | EVQLL | 5406 | FTFSSY | 362 | WVRQAP | 437 | SI- | 5980 | RFTISR | 6291 | ARDSRGL | 6596 | WGQGTL |
| | | 21 | ESGGG | | SMN | | GKGLEW | | LVTSHV | | DNVKNS | | YGSGIPD | | VTVSS |
| | | | LVKPG | | | | VS | | FYADSV | | LYLQMN | | F | | |
| | | | GSLRL | | | | | | KG | | SLRAED | | | | |
| | | | SCAAS | | | | | | | | TAVYYC | | | | |
| | | | G | | | | | | | | | | | | |
| ADI-81890 | 66 | IGHV3- | RCSCW | 5407 | FTVSAN | 5614 | WVRQAP | 437 | VIYSDG | 5981 | RFTVSR | 6292 | ARVVSDA | 6597 | WGQGTT |
| | | 53 | SPGGG | | YMT | | GKGLEW | | NTYYAN | | DNSKNT | | FDL | | VTVSS |
| | | | LIQRG | | | | VS | | SVKG | | LFLQMS | | | | |
| | | | GSLRL | | | | | | | | SLRAED | | | | |
| | | | SCVAS | | | | | | | | TAVYFC | | | | |
| | | | G | | | | | | | | | | | | |
| ADI-81612 | 69 | IGHV1- | QVQLQ | 5408 | GTFSTY | 5615 | WLRQAP | 478 | GIIPIV | 5982 | RVTITA | 6293 | ARGSGYS | 6598 | WGQGTL |
| | | 69 | QSGAE | | TVT | | GQGLEW | | GIPNYA | | DKSTST | | ASGSNTY | | VTVSS |
| | | | VKKPG | | | | MG | | ENFQG | | AYMELS | | FDP | | |
| | | | SSVKV | | | | | | | | SLKSED | | | | |
| | | | SCRAS | | | | | | | | TALYYC | | | | |
| | | | G | | | | | | | | | | | | |
| ADI-81723 | 93 | IGHV4- | QVQLQ | 5409 | YSIRSD | 5616 | WIRQTP | 5820 | SVYYSG | 5983 | RVTISV | 6294 | ARNSSRT | 6599 | WGQGTL |
| | | 38-2 | QSGPG | | DYWG | | GKGLEW | | STHYNP | | DTSKNQ | | PLFDY | | VTVSS |
| | | | LVKPS | | | | IG | | SLKS | | FSLSLT | | | | |
| | | | DTLSL | | | | | | | | SVTAAD | | | | |
| | | | TCAVS | | | | | | | | TAVYYC | | | | |
| | | | G | | | | | | | | | | | | |
| ADI-81543 | 11 | IGHV1- | QVQLV | 5410 | GTFGSD | 5617 | WVRQAP | 433 | RIIPIL | 5984 | RVTITA | 6295 | AREGGTT | 6600 | WGQGTL |
| | | 69 | QSGGE | | SIS | | GQGLEW | | GVPNSA | | DKSTST | | TEYFRQ | | VTVSS |
| | | | VKKPG | | | | MG | | QKFQD | | AYMELS | | | | |
| | | | SSVKV | | | | | | | | SLRYDD | | | | |
| | | | SCKAS | | | | | | | | TAVYFC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81697 | 1286 | CGAGGTGCAGCTGCAGGAGTCGGGCCCAGGACTGGTGAGGC | | | | | | 6956 | RGAAAGVGPRTGEA | | 7307 | 15 | | 10 | |
| | | TTCGGGGACCCTGTCCCTCACCTGCGCTGTCTCAGGTGCCT | | | | | | | SGTLSLTCAVSGAS | | | | | | |
| | | CCATCAGCAGTAGTAACTGGTGGAGTTGGGTCCGCCAGCCC | | | | | | | ISSSNWWSWVRQPP | | | | | | |
| | | CCAGGGAAGGGGCTGGAGTGGATCGGGGAAATCTATCATGT | | | | | | | GKGLEWIGEIYHVG | | | | | | |
| | | TGGGAGTACCAACTACAACCCGTCCCTCAAGAGTCGAGTCA | | | | | | | STNYNPSLKSRVTI | | | | | | |
| | | CCATATCAATAGACAAGTCCAACAACCAGTTCTCCCTGAAG | | | | | | | SIDKSNNQFSLKLN | | | | | | |
| | | CTGAACTCTGTGACCGCCGCGGACACGGCCGTGTACTACTG | | | | | | | SVTAADTAVYYCAR | | | | | | |
| | | TGCGAGAGTCGTACTCTCTGACTTCAAGGAGGCGGCGTCGT | | | | | | | VVLSDFKEAASWGQ | | | | | | |
| | | GGGGCCAGGGAACCCTGGTCACCGTCTCCTCA | | | | | | | GTLVTVSS | | | | | | |
| ADI-81549 | 1286 | CAGGTCCAGCTGGTGCAGTCTGGGGGAGGCTTGGTACAGCC | | | | | | 6957 | QVQLVQSGGGLVQP | | 7308 | 14 | | 11 | |
| | | GGGGGGGTCCCTGAGACTCTCCTGTACAGCCTCTGGATTCA | | | | | | | GGSLRLSCTASGFS | | | | | | |
| | | GCTTTAGTCTGTTTGCCATGAACTGGGTCCGCCAGGCTCCA | | | | | | | FSLFAMNWVRQAPG | | | | | | |
| | | GGAAAGGGGCTGGAGTGGGTCTCAAGTATTACTGGAAGTGC | | | | | | | KGLEWVSSITGSAD | | | | | | |
| | | TGATACCACATACTACGCAGACTCCGTGAAGGGCCGGTTCA | | | | | | | TTYYADSVKGRFTI | | | | | | |
| | | CCATATCCAGAGACAATTCCAAGAACACACTGTATCTGCAA | | | | | | | SRDNSKNTLYLQMN | | | | | | |
| | | ATGAACAGCCTGAGCGCCGAGGACACGGCCGTATATTATTG | | | | | | | SLSAEDTAVYYCAK | | | | | | |
| | | TGCGAAAGGGATGGACGGTGCCCCGGGGTTCTACTTTGACT | | | | | | | GMDGAPGFYFDYWG | | | | | | |
| | | ACTGGGGCCAGGGAACCCTGGTCACCGTCTCCTCA | | | | | | | QGTLVTVSS | | | | | | |
| ADI-81562 | 1287 | CAGGTGCAGCTGGTGGAGTCTGGAGGAGGCTTGGTCCAGCC | | | | | | 6958 | QVQLVESGGGLVQP | | 7309 | 14 | | 11 | |
| | | TGGGGGGTCCCTGAGACTCTCCTGTGCAGCCTCTGGGCTCA | | | | | | | GGSLRLSCAASGLT | | | | | | |
| | | CCGTCAGTAGCAACTATATGACCTGGCTTCGCCAGGCTCCA | | | | | | | VSSNYMTWLRQAPG | | | | | | |
| | | GGGAAGGGGCTAGAGTGGGTCTCAGTTATGTTTAGCGGTGG | | | | | | | KGLEWVSVMFSGGS | | | | | | |
| | | TAGCACATTCTACGCAGACTCCGTGAAGGGCCGATTCACCA | | | | | | | TFYADSVKGRFTIS | | | | | | |
| | | TCTCCAGACACAATTCCAACAACACGCTGTATCTTCAAATG | | | | | | | RHNSNNTLYLQMSS | | | | | | |
| | | AGCAGCCTGGGACCTGAGGACACGGCCACATATTACTGTGC | | | | | | | LGPEDTATYYCARD | | | | | | |
| | | AAGAGATCTGCCTCCATACGGTATGGACGTCTGGGGCCAAG | | | | | | | LPPYGMDVWGQGTT | | | | | | |
| | | GGACCACGGTCACCGTCTCCTCA | | | | | | | VTVSS | | | | | | |
| ADI-81813 | 1286 | GAGGTGCAGCTGTTGGAGTCTGGGGGAGGCCTGGTCAAGCC | | | | | | 6959 | EVQLLESGGGLVKP | | 7310 | 13 | | 8 | |
| | | TGGGGGGTCCCTGAGACTCTCCTGTGCAGCCTCTGGATTCA | | | | | | | GGSLRLSCAASGFT | | | | | | |
| | | CCTTCAGTAGTTATAGCATGAACTGGGTCCGCCAGGCTCCA | | | | | | | FSSYSMNWVRQAPG | | | | | | |
| | | GGGAAGGGGCTGGAGTGGGTCTCTTCCATTCTTGTCACTAG | | | | | | | KGLEWVSSILVTSH | | | | | | |
| | | CCACGTATTCTACGCAGACTCAGTGAAGGGCCGATTCACCA | | | | | | | VFYADSVKGRFTIS | | | | | | |
| | | TTTCCAGAGACAACGTCAAGAACTCACTGTATCTGCAAATG | | | | | | | RDNVKNSLYLQMNS | | | | | | |
| | | AACAGCCTGAGAGCCGAGGATACGGCTGTCTATTACTGTGC | | | | | | | LRAEDTAVYYCARD | | | | | | |
| | | GAGAGATTCCCGTGGGCTCTATGGGTCAGGGATCCCCGACT | | | | | | | SRGLYGSGIPDFWG | | | | | | |
| | | TCTGGGGCCAGGGAACCCTGGTCACCGTCTCCTCA | | | | | | | QGTLVTVSS | | | | | | |
| ADI-81890 | 1287 | AGGTGCAGCTGTTGGAGTCCTGGAGGAGGCTTGATCCAGCG | | | | | | 6960 | RCSCWSPGGGLIQR | | 7311 | 13 | | 11 | |
| | | GGGGGGGTCCCTGAGACTCTCCTGTGTAGCCTCTGGGTTCA | | | | | | | GGSLRLSCVASGFT | | | | | | |
| | | CCGTCAGTGCCAACTACATGACCTGGGTCCGCCAGGCTCCA | | | | | | | VSANYMTWVRQAPG | | | | | | |
| | | GGGAAGGGACTCGAGTGGGTCTCAGTGATTTATAGTGATGG | | | | | | | KGLEWVSVIYSDGN | | | | | | |
| | | TAACACATACTATGCAAACTCCGTGAAGGGCCGGTTCACCG | | | | | | | TYYANSVKGRFTVS | | | | | | |
| | | TCTCCAGAGACAATTCCAAGAACACGCTGTTTCTTCAAATG | | | | | | | RDNSKNTLFLQMSS | | | | | | |
| | | AGCAGCCTGAGAGCCGAGGACACGGCCGTGTATTTCTGTGC | | | | | | | LRAEDTAVYFCARV | | | | | | |
| | | GAGAGTCGTGTCGGATGCTTTTGATCTCTGGGGCCAAGGGA | | | | | | | VSDAFDLWGQGTTV | | | | | | |
| | | CCACGGTCACCGTCTCCTCA | | | | | | | TVSS | | | | | | |
| ADI-81612 | 1286 | TGGGTCCTCGGTGAAGGTCTCCTGCAGGGCTTCTGGAGGCA | | | | | | 6961 | QVQLQQSGAEVKKP | | 7312 | 12 | | 12 | |
| | | CCTTCAGCACCTATACTGTCACCTGGCTGCGACAGGCCCCT | | | | | | | GSSVKVSCRASGGT | | | | | | |
| | | GGACAAGGGCTTGAGTGGATGGGAGGGATCATCCCTATCGT | | | | | | | FSTYTVTWLRQAPG | | | | | | |
| | | TGGAATACCAAACTACGCAGAGAACTTCCAGGGCAGAGTCA | | | | | | | QGLEWMGGIIPIVG | | | | | | |
| | | CGATTACCGCGGACAAATCCACGAGCACAGCCTACATGGAG | | | | | | | IPNYAENFQGRVTI | | | | | | |
| | | CTGAGCAGCCTGAAATCTGAGGACACGGCCCTGTATTACTG | | | | | | | TADKSTSTAYMELS | | | | | | |
| | | TGCGAGAGGTTCGGGATACTCTGCTTCAGGGAGTAATACCT | | | | | | | SLKSEDTALYYCAR | | | | | | |
| | | ACTTCGACCCCTGGGGCCAGGGAACCCTGGTCACCGTCTCC | | | | | | | GSGYSASGSNTYFD | | | | | | |
| | | TCA | | | | | | | PWGQGTLVTVSS | | | | | | |
| ADI-81723 | 1286 | CAGGTGCAGCTGCAGCAGTCGGGCCCGGGACTGGTGAAGCC | | | | | | 6962 | QVQLQQSGPGLVKP | | 7313 | 12 | | 10 | |
| | | TTCGGACACCCTGTCCCTCACCTGCGCTGTCTCTGGTTACT | | | | | | | SDTLSLTCAVSGYS | | | | | | |
| | | CCATCAGGAGTGACGACTACTGGGGCTGGATCCGGCAGACC | | | | | | | IRSDDYWGWIRQTP | | | | | | |
| | | CCAGGGAAGGGGCTGGAGTGGATTGGGAGTGTCTATTATAG | | | | | | | GKGLEWIGSVYYSG | | | | | | |
| | | TGGCAGCACCCACTACAACCCGTCCCTCAAGAGTCGAGTCA | | | | | | | STHYNPSLKSRVTI | | | | | | |
| | | CCATATCAGTAGACACGTCCAAGAACCAGTTCTCCCTGAGT | | | | | | | SVDTSKNQFSLSLT | | | | | | |
| | | CTGACCTCTGTGACCGCCGCAGACACGGCCGTGTATTACTG | | | | | | | SVTAADTAVYYCAR | | | | | | |
| | | TGCGCGAAACTCTTCAAGAACTCCCTTATTTGACTACTGGG | | | | | | | NSSRTPLFDYWGQG | | | | | | |
| | | GCCAGGGAACCCTGGTCACCGTCTCCTCA | | | | | | | TLVTVSS | | | | | | |
| ADI-81543 | 1286 | CAGGTGCAGCTGGTGCAGTCTGGGGGTGAGGTGAAGAAGCC | | | | | | 6963 | QVQLVQSGGEVKKP | | 7314 | 11 | | 10 | |
| | | TGGGTCCTCGGTGAAGGTCTCCTGCAAGGCTTCGGGAGGCA | | | | | | | GSSVKVSCKASGGT | | | | | | |
| | | CCTTCGGCAGTGACTCTATCAGCTGGGTGCGACAGGCCCCT | | | | | | | FGSDSISWVRQAPG | | | | | | |
| | | GGACAAGGGCTTGAGTGGATGGGAAGGATCATCCCTATCCT | | | | | | | QGLEWMGRIIPILG | | | | | | |
| | | CGGTGTACCAAACTCCGCACAGAAGTTCCAGGACAGAGTCA | | | | | | | VPNSAQKFQDRVTI | | | | | | |
| | | CAATTACCGCGGACAAATCCACGAGCACAGCCTACATGGAG | | | | | | | TADKSTSTAYMELS | | | | | | |
| | | CTGAGCAGCCTGAGATATGACGACACGGCCGTGTATTTCTG | | | | | | | SLRYDDTAVYFCAR | | | | | | |
| | | TGCGAGAGAAGGTGGAACCACCACTGAATACTTCCGGCAGT | | | | | | | EGGTTTEYFRQWGQ | | | | | | |
| | | GGGGCCAGGGCACCCTGGTCACCGTCTCCTCA | | | | | | | GTLVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81583 | 44 | IGHV3- | QVQLV | 57 | FTFSNY | 290 | WVRQAP | 431 | VISSDG | 5985 | RFTISR | 6296 | ARDPPTS | 6601 | WGQGTL |
| | | 30 | QSGGG | | DMH | | GKGLEW | | SLRFYA | | DNSKNT | | VTSQMLL | | VTVSS |
| | | | VVQPG | | | | VA | | DSVKG | | LYLQMN | | DF | | |
| | | | GSLRL | | | | | | | | SLRTED | | | | |
| | | | SCAAS | | | | | | | | TAVFYC | | | | |
| | | | G | | | | | | | | | | | | |
| ADI-81620 | 77 | IGHV3- | QVQLV | 12 | IIVSSN | 5618 | WVRQAP | 437 | LMYAGG | 5986 | RFTISR | 6297 | ARDLAEP | 6602 | WGQGTT |
| | | 66 | QSGGG | | YMS | | GKGLEW | | STFYAD | | DNSKNT | | YGMDV | | VTVSS |
| | | | LVQPG | | | | VS | | SVKG | | LFLQMN | | | | |
| | | | GSLRL | | | | | | | | SLRVED | | | | |
| | | | SCAAS | | | | | | | | TAVYYC | | | | |
| | | | G | | | | | | | | | | | | |
| ADI-81743 | 15 | IGHV5- | EVQLV | 5411 | YTFSTY | 5619 | WVRQMP | 476 | IIFPGD | 5987 | QVTISA | 6298 | ATLKLEG | 6603 | WGQGTL |
| | | 51 | QSGAE | | WIG | | GKGLEW | | SDTRYS | | DKSIST | | FTPVDYN | | VTVSS |
| | | | VRKPG | | | | MG | | PSFQG | | AYVQWS | | TLPALY | | |
| | | | ESLKI | | | | | | | | SLKASD | | | | |
| | | | SCKAS | | | | | | | | TAMYYC | | | | |
| | | | G | | | | | | | | | | | | |
| ADI-81795 | 71 | IGHV3- | EVQLL | 5412 | FIFNDY | 5620 | WVRQVP | 436 | GISWNG | 5988 | RFTISR | 6299 | AKDKRLD | 6604 | WGQGTL |
| | | 9 | ESGGG | | AMH | | GKGLEW | | VSIGYG | | DNAKNS | | YSNYGTA | | VTVSS |
| | | | LVQPG | | | | VS | | DSVKG | | LYLHMN | | PEY | | |
| | | | RSLRL | | | | | | | | SLRGED | | | | |
| | | | SCAAA | | | | | | | | TAVYYC | | | | |
| | | | G | | | | | | | | | | | | |
| ADI-81812 | 86 | IGHV3- | QVQLV | 30 | LIVSSN | 343 | WVRQAP | 5821 | TLYSGG | 5989 | RFTISR | 6300 | ARDALYY | 6605 | WGQGTL |
| | | 53 | ESGGG | | YMS | | GKGLAW | | STFYAD | | DNSKNT | | GDSN | | VTVSS |
| | | | LIQPG | | | | VS | | SVRG | | LYLQMN | | | | |
| | | | GSLRL | | | | | | | | SLTGED | | | | |
| | | | SCAAS | | | | | | | | TAVYYC | | | | |
| | | | G | | | | | | | | | | | | |
| ADI-81877 | 54 | IGHV3- | EVQLL | 5324 | FTFDDY | 336 | WVRQAP | 437 | GSTWNS | 5990 | RFIISR | 6301 | ARDSTIL | 6606 | WGQGTL |
| | | 9 | ESGGG | | AIH | | GKGLEW | | GTIGYA | | DNARNS | | GAKPEEY | | VTVSS |
| | | | LVQPG | | | | VS | | DSVKG | | LYLQMN | | YFDY | | |
| | | | RSLRL | | | | | | | | RLRPED | | | | |
| | | | SCAAS | | | | | | | | TAFYYC | | | | |
| | | | G | | | | | | | | | | | | |
| ADI-81577 | 2 | IGHV3- | RCTLV | 5413 | FTFSSD | 5621 | WVRQAP | 431 | LISSDG | 5991 | RFTISR | 6302 | ARHQESV | 6607 | WGRGTT |
| | | 30 | ESGGG | | DIY | | GKGLEW | | SNKYYA | | DNSKNT | | LPTADRP | | VTVSS |
| | | | VVQPG | | | | VA | | DSVKG | | LYLQMN | | YRYYYYM | | |
| | | | RSLRL | | | | | | | | SLRGDD | | DV | | |
| | | | SCAAS | | | | | | | | TAVYHC | | | | |
| | | | G | | | | | | | | | | | | |
| ADI-81742 | 19 | IGHV2- | QVTLK | 5414 | FSLSTN | 5622 | WIRQPP | 441 | LIYWDD | 5992 | RLTITK | 6303 | ARFKEHL | 6608 | WGQGTL |
| | | 5 | ESGPT | | GVGVA | | GKALEW | | DKRFSP | | DTSKNQ | | GYNWFDP | | VTVSS |
| | | | LVKPT | | | | LA | | SLRN | | VVLTLT | | | | |
| | | | QTLTL | | | | | | | | NIDPVD | | | | |
| | | | TCSFS | | | | | | | | TATYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81583 | 1286 | CAGGTCCAGCTGGTGCAGTCTGGGGGAGGTGTGGTCCAGCC | | | | | | 6964 | QVQLVQSGGGVVQP | | 7315 | 10 | | 9 | |
| | | TGGGGGGTCCCTGAGACTCTCCTGTGCAGCCTCTGGATTCA | | | | | | | GGSLRLSCAASGFT | | | | | | |
| | | CCTTCAGTAACTATGATATGCACTGGGTCCGCCAGGCTCCA | | | | | | | FSNYDMHWVRQAPG | | | | | | |
| | | GGCAAGGGGCTAGAGTGGGTGGCAGTCATATCATCTGATGG | | | | | | | KGLEWVAVISSDGS | | | | | | |
| | | AAGTCTTAGATTCTACGCAGACTCCGTGAAGGGCCGATTCA | | | | | | | LRFYADSVKGRFTI | | | | | | |
| | | CCATCTCCAGGGACAATTCCAAGAACACTCTATATCTGCAA | | | | | | | SRDNSKNTLYLQMN | | | | | | |
| | | ATGAACAGCCTGAGAACTGAGGACACGGCTGTTTTTTACTG | | | | | | | SLRTEDTAVFYCAR | | | | | | |
| | | TGCGAGAGATCCCCCCACTTCGGTGACTTCCCAGATGCTTC | | | | | | | DPPTSVTSQMLLDF | | | | | | |
| | | TTGACTTCTGGGGCCAGGGAACCCTGGTCACCGTCTCCTCA | | | | | | | WGQGTLVTVSS | | | | | | |
| ADI-81620 | 1287 | CAGGTGCAGCTGGTGCAGTCTGGGGGAGGCTTGGTCCAGCC | | | | | | 6965 | QVQLVQSGGGLVQP | | 7316 | 10 | | 8 | |
| | | TGGGGGGTCCCTGAGACTCTCCTGTGCAGCCTCTGGAATCA | | | | | | | GGSLRLSCAASGII | | | | | | |
| | | TCGTCAGTAGCAACTACATGAGTTGGGTCCGCCAGGCTCCA | | | | | | | VSSNYMSWVRQAPG | | | | | | |
| | | GGGAAGGGGCTGGAGTGGGTCTCACTGATGTATGCCGGTGG | | | | | | | KGLEWVSLMYAGGS | | | | | | |
| | | TAGTACATTCTACGCAGACTCCGTGAAGGGCAGATTCACCA | | | | | | | TFYADSVKGRFTIS | | | | | | |
| | | TCTCCAGAGACAATTCCAAAAACACGCTGTTTCTTCAAATG | | | | | | | RDNSKNTLFLQMNS | | | | | | |
| | | AACAGCCTGAGAGTCGAGGACACGGCTGTGTATTACTGTGC | | | | | | | LRVEDTAVYYCARD | | | | | | |
| | | GAGGGATTTGGCAGAGCCCTACGGTATGGACGTCTGGGGCC | | | | | | | LAEPYGMDVWGQGT | | | | | | |
| | | AAGGGACCACGGTCACCGTCTCCTCA | | | | | | | TVTVSS | | | | | | |
| ADI-81743 | 1286 | GAGGTGCAGCTGGTGCAGTCTGGAGCAGAGGTGAGAAAGCC | | | | | | 6966 | EVQLVQSGAEVRKP | | 7317 | 10 | | 7 | |
| | | CGGGGAGTCTCTGAAGATCTCCTGTAAGGCCTCTGGATACA | | | | | | | GESLKISCKASGYT | | | | | | |
| | | CCTTTAGTACCTACTGGATCGGCTGGGTGCGCCAGATGCCC | | | | | | | FSTYWIGWVRQMPG | | | | | | |
| | | GGGAAAGGCCTGGAGTGGATGGGGATCATCTTTCCTGGTGA | | | | | | | KGLEWMGIIFPGDS | | | | | | |
| | | CTCTGATACCAGGTACAGCCCGTCCTTCCAAGGCCAGGTCA | | | | | | | DTRYSPSFQGQVTI | | | | | | |
| | | CCATCTCAGCCGACAAGTCCATCAGCACCGCCTACGTACAG | | | | | | | SADKSISTAYVQWS | | | | | | |
| | | TGGAGCAGCCTGAAGGCCTCGGACACCGCCATGTATTACTG | | | | | | | SLKASDTAMYYCAT | | | | | | |
| | | TGCGACTCTGAAACTCGAGGGATTTACCCCCGTTGATTACA | | | | | | | LKLEGFTPVDYNTL | | | | | | |
| | | ACACACTTCCCGCACTTTACTGGGGCCAGGGAACCCTGGTC | | | | | | | PALYWGQGTLVTVS | | | | | | |
| | | ACCGTCTCCTCA | | | | | | | S | | | | | | |
| ADI-81795 | 1286 | TGGCAGGTCCCTGAGACTCTCATGTGCAGCCGCTGGATTCA | | | | | | 6967 | EVQLLESGGGLVQP | | 7318 | 10 | | 10 | |
| | | TCTTTAATGATTATGCCATGCACTGGGTCCGGCAAGTTCCA | | | | | | | GRSLRLSCAAAGFI | | | | | | |
| | | GGGAAGGGCCTGGAGTGGGTCTCAGGTATTAGTTGGAATGG | | | | | | | FNDYAMHWVRQVPG | | | | | | |
| | | TGTTAGCATAGGCTATGGGGACTCTGTGAAGGGCCGATTCA | | | | | | | KGLEWVSGISWNGV | | | | | | |
| | | CCATCTCCAGAGACAACGCCAAGAACTCCCTGTATCTGCAC | | | | | | | SIGYGDSVKGRFTI | | | | | | |
| | | ATGAACAGTCTGAGAGGTGAGGATACGGCCGTGTATTACTG | | | | | | | SRDNAKNSLYLHMN | | | | | | |
| | | TGCAAAAGATAAGAGACTTGACTACAGTAACTACGGGACTG | | | | | | | SLRGEDTAVYYCAK | | | | | | |
| | | CCCCTGAGTACTGGGGCCAGGGAACCCTGGTCACCGTCTCC | | | | | | | DKRLDYSNYGTAPE | | | | | | |
| | | TCA | | | | | | | YWGQGTLVTVSS | | | | | | |
| ADI-81812 | 1286 | CAGGTGCAGCTGGTGGAGTCTGGAGGAGGCTTGATCCAGCC | | | | | | 6968 | QVQLVESGGGLIQP | | 7319 | 10 | | 9 | |
| | | TGGGGGGTCCCTGAGACTCTCCTGTGCAGCCTCTGGGCTCA | | | | | | | GGSLRLSCAASGLI | | | | | | |
| | | TCGTCAGTAGCAACTACATGAGCTGGGTCCGCCAGGCTCCA | | | | | | | VSSNYMSWVRQAPG | | | | | | |
| | | GGGAAGGGGCTGGCGTGGGTCTCAACTCTTTATAGCGGTGG | | | | | | | KGLAWVSTLYSGGS | | | | | | |
| | | TAGCACATTCTACGCAGACTCCGTGAGGGGCCGGTTCACCA | | | | | | | TFYADSVRGRFTIS | | | | | | |
| | | TCTCCAGAGACAACTCCAAGAACACGCTGTATCTTCAAATG | | | | | | | RDNSKNTLYLQMNS | | | | | | |
| | | AACAGCCTGACAGGCGAGGACACGGCCGTTTATTACTGTGC | | | | | | | LTGEDTAVYYCARD | | | | | | |
| | | GAGAGATGCCCTATATTACGGTGACTCAAACTGGGGCCAGG | | | | | | | ALYYGDSNWGQGTL | | | | | | |
| | | GAACCCTGGTCACCGTCTCCTCA | | | | | | | VTVSS | | | | | | |
| ADI-81877 | 1286 | TGGCAGGTCCCTGAGACTCTCCTGTGCAGCCTCTGGATTCA | | | | | | 6969 | EVQLLESGGGLVQP | | 7320 | 10 | | 9 | |
| | | CCTTTGATGATTATGCCATACACTGGGTCCGGCAAGCTCCA | | | | | | | GRSLRLSCAASGFT | | | | | | |
| | | GGGAAGGGCCTGGAGTGGGTCTCAGGTAGTACTTGGAATAG | | | | | | | FDDYAIHWVRQAPG | | | | | | |
| | | TGGTACTATAGGCTATGCGGACTCTGTGAAGGGCCGATTCA | | | | | | | KGLEWVSGSTWNSG | | | | | | |
| | | TCATCTCCAGAGACAACGCCAGGAACTCCCTATATCTGCAA | | | | | | | TIGYADSVKGRFII | | | | | | |
| | | ATGAATCGTCTGAGACCTGAGGACACGGCCTTCTATTACTG | | | | | | | SRDNARNSLYLQMN | | | | | | |
| | | TGCAAGAGATTCTACAATACTGGGAGCTAAGCCCGAAGAGT | | | | | | | RLRPEDTAFYYCAR | | | | | | |
| | | ATTACTTTGACTATTGGGGCCAGGGAACCCTGGTCACCGTC | | | | | | | DSTILGAKPEEYYF | | | | | | |
| | | TCCTCA | | | | | | | DYWGQGTLVTVSS | | | | | | |
| ADI-81577 | 1294 | AGGTGCACGCTGGTGGAGTCTGGGGGAGGCGTGGTCCAGCC | | | | | | 6970 | RCTLVESGGGVVQP | | 7321 | 9 | | 9 | |
| | | TGGGAGGTCCCTGAGACTCTCCTGTGCAGCCTCTGGATTCA | | | | | | | GRSLRLSCAASGFT | | | | | | |
| | | CCTTCAGTAGTGATGATATATACTGGGTCCGCCAGGCTCCA | | | | | | | FSSDDIYWVRQAPG | | | | | | |
| | | GGCAAGGGGCTAGAATGGGTGGCACTTATATCTTCTGATGG | | | | | | | KGLEWVALISSDGS | | | | | | |
| | | AAGTAATAAGTACTACGCAGACTCCGTGAAGGGCCGATTCA | | | | | | | NKYYADSVKGRFTI | | | | | | |
| | | CCATCTCCAGAGACAATTCCAAGAACACGCTGTATCTGCAA | | | | | | | SRDNSKNTLYLQMN | | | | | | |
| | | ATGAACAGCCTGAGAGGTGACGACACGGCTGTGTATCACTG | | | | | | | SLRGDDTAVYHCAR | | | | | | |
| | | TGCGAGACATCAGGAGTCCGTACTCCCAACTGCTGATAGGC | | | | | | | HQESVLPTADRPYR | | | | | | |
| | | CCTACCGTTACTACTACTACATGGACGTCTGGGGCAGAGGG | | | | | | | YYYYMDVWGRGTTV | | | | | | |
| | | ACCACGGTCACCGTCTCCTCA | | | | | | | TVSS | | | | | | |
| ADI-81742 | 1286 | CAGGTCACGTTGAAGGAGTCTGGTCCTACGCTGGTGAAACC | | | | | | 6971 | QVTLKESGPTLVKP | | 7322 | 9 | | 8 | |
| | | CACACAGACCCTCACGCTGACCTGCAGCTTCTCTGGGTTCT | | | | | | | TQTLTLTCSFSGFS | | | | | | |
| | | CACTCAGCACTAATGGAGTGGGTGTTGCCTGGATCCGTCAG | | | | | | | LSTNGVGVAWIRQP | | | | | | |
| | | CCCCCAGGAAAGGCCCTGGAGTGGCTTGCACTCATTTATTG | | | | | | | PGKALEWLALIYWD | | | | | | |
| | | GGATGATGATAAGCGTTTCAGCCCATCTCTGAGAAACAGAC | | | | | | | DDKRFSPSLRNRLT | | | | | | |
| | | TCACAATCACCAAGGACACCTCCAAAAACCAGGTGGTCCTT | | | | | | | ITKDTSKNQVVLTL | | | | | | |
| | | ACACTGACCAACATTGACCCTGTGGACACAGCCACATATTA | | | | | | | TNIDPVDTATYYCA | | | | | | |
| | | CTGTGCACGCTTCAAGGAGCACCTCGGGTACAACTGGTTCG | | | | | | | RFKEHLGYNWFDPW | | | | | | |
| | | ACCCCTGGGGCCAGGGAACCCTGGTCACCGTCTCCTCA | | | | | | | GQGTLVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81781 | 57 | IGHV1- | EVQLV | 5320 | YTFTGH | 5623 | WVRQAP | 433 | RINPNS | 5993 | RVTMTR | 6304 | AVEPYYN | 6609 | WGQGTL |
| | | 2 | QSGAE | | YMY | | GQGLEW | | GDTHYA | | DASIST | | ILTASYR | | VTVSS |
| | | | VKKPG | | | | MG | | QKFQG | | AFMELS | | DC | | |
| | | | ASVKV | | | | | | | | RLRSDD | | | | |
| | | | SCKAS | | | | | | | | TAVYYC | | | | |
| | | | G | | | | | | | | | | | | |
| ADI-81611 | 68 | IGHV3- | EVQLV | 5415 | FTFGDH | 5624 | WVRQAP | 432 | FIRTKT | 5994 | RFTISR | 6305 | TGVLGWR | 6610 | WGQGTL |
| | | 49 | ESGGG | | AMN | | GKGLEW | | YGGTTE | | DDSKSI | | GSYREDY | | VTVSS |
| | | | LVQPG | | | | VG | | YAASVK | | AYLQMN | | | | |
| | | | RSLRL | | | | | | G | | SLRTED | | | | |
| | | | SCITS | | | | | | | | TAVYYC | | | | |
| | | | G | | | | | | | | | | | | |
| ADI-81622 | 79 | IGHV4- | QVQLQ | 195 | GSISSR | 5625 | WIRQSP | 5822 | SIYYSG | 5995 | RVTISV | 6306 | ARRPTTD | 6611 | WGKGTT |
| | | 39 | QSGPG | | SYYWD | | GRGLEW | | SADYNP | | DTSKNH | | FYYMDV | | VTVSS |
| | | | LVKPS | | | | IG | | SLKS | | FSLKLS | | | | |
| | | | ETLSL | | | | | | | | SVTAAD | | | | |
| | | | TCTVS | | | | | | | | TAVYYC | | | | |
| | | | G | | | | | | | | | | | | |
| ADI-81644 | 17 | IGHV3- | QVQLL | 5416 | FTFDDY | 214 | WVRQAP | 437 | GITWNS | 5996 | RFIIIR | 6307 | VKDSREI | 6612 | WGKGTT |
| | | 9 | ESGGG | | AMH | | GKGLEW | | GTIGYA | | DNAKNS | | KTLMVEE | | VTVSS |
| | | | LVQPG | | | | VS | | DSVKG | | LYLQMN | | DYYMDV | | |
| | | | RSLRL | | | | | | | | SLRVED | | | | |
| | | | TCAAS | | | | | | | | TALYYC | | | | |
| | | | G | | | | | | | | | | | | |
| ADI-81858 | 36 | IGHV3- | EVQLV | 5338 | FTFINY | 5626 | WVRQAP | 431 | AISYDG | 5997 | RFTISR | 6308 | AKVTASH | 6613 | WGQGTT |
| | | 30 | ESGGG | | GMH | | GKGLEW | | SDEYYA | | DDSKNT | | YYYYYGM | | VTVSS |
| | | | VVQPG | | | | VA | | DSVKG | | VYLQMN | | DV | | |
| | | | RSLRL | | | | | | | | SLRAED | | | | |
| | | | SCAAS | | | | | | | | TAVYYC | | | | |
| | | | G | | | | | | | | | | | | |
| ADI-81603 | 61 | IGHV1- | QVQLV | 50 | GTFSSY | 379 | WVRQAP | 433 | RIIPPL | 5998 | RVTITA | 6224 | ARDRGYS | 6614 | WGKGTT |
| | | 69 | QSGAE | | PIS | | GQGLEW | | GISDYA | | DKSTST | | SVSGSNY | | VTVSS |
| | | | VKKPG | | | | MG | | QKFQG | | AYMELS | | YMDV | | |
| | | | SSVKV | | | | | | | | SLTSED | | | | |
| | | | SCKAS | | | | | | | | TAVYYC | | | | |
| | | | G | | | | | | | | | | | | |
| ADI-81535 | 4 | IGHV1- | QVQLV | 50 | GTFSSN | 5627 | WVRQAP | 433 | RIIPIL | 5999 | RVTFTA | 6309 | ARDAGYS | 6615 | WGQGTL |
| | | 69 | QSGAE | | PFS | | GQGLEW | | GIPNYA | | DKSTST | | GSGSAYY | | VTVSS |
| | | | VKKPG | | | | MG | | QKFQG | | AYMELS | | FDY | | |
| | | | SSVKV | | | | | | | | SLRSED | | | | |
| | | | SCKAS | | | | | | | | TAVYYC | | | | |
| | | | G | | | | | | | | | | | | |
| ADI-81713 | 83 | IGHV1- | QVQLV | 137 | FTFITS | 5575 | WVRQAR | 5789 | WIAVGR | 6000 | RVTITR | 6310 | AALNCNV | 6616 | WGQGTT |
| | | 58 | QSGPE | | AVQ | | GQRLEW | | GNTDYA | | DMSTST | | TTCSDGF | | VTVSS |
| | | | VKKPG | | | | IG | | QKFQE | | AYMELS | | DM | | |
| | | | TSVKV | | | | | | | | SLRSED | | | | |
| | | | SCKAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81781 | 1286 | GAGGTGCAGCTGGTGCAGTCTGGGGCTGAGGTGAAGAAGCC | | | | | | 6972 | EVQLVQSGAEVKKP | | 7323 | 8 | | 6 | |
| | | TGGGGCCTCAGTGAAGGTCTCCTGCAAGGCTTCTGGATACA | | | | | | | GASVKVSCKASGYT | | | | | | |
| | | CCTTCACCGGCCATTATATGTACTGGGTGCGACAGGCCCCT | | | | | | | FTGHYMYWVRQAPG | | | | | | |
| | | GGACAAGGGCTTGAGTGGATGGGACGGATCAACCCCAACAG | | | | | | | QGLEWMGRINPNSG | | | | | | |
| | | TGGTGACACACACTATGCACAGAAGTTTCAGGGCAGGGTCA | | | | | | | DTHYAQKFQGRVTM | | | | | | |
| | | CCATGACCAGGGACGCCTCCATCAGCACAGCCTTCATGGAG | | | | | | | TRDASISTAFMELS | | | | | | |
| | | CTGAGCAGGCTGAGATCTGACGACACGGCCGTGTATTACTG | | | | | | | RLRSDDTAVYYCAV | | | | | | |
| | | TGCGGTCGAGCCTTATTACAATATTTTGACTGCTTCTTATC | | | | | | | EPYYNILTASYRDC | | | | | | |
| | | GAGACTGCTGGGGCCAGGGAACCCTGGTCACCGTCTCCTCA | | | | | | | WGQGTLVTVSS | | | | | | |
| ADI-81611 | 1286 | GAGGTGCAGCTGGTGGAGTCTGGGGGAGGCTTGGTACAGCC | | | | | | 6973 | EVQLVESGGGLVQP | | 7324 | 7 | | 7 | |
| | | AGGGCGGTCCCTAAGACTCTCCTGTATAACTTCTGGATTCA | | | | | | | GRSLRLSCITSGFT | | | | | | |
| | | CCTTTGGTGATCATGCTATGAACTGGGTCCGCCAGGCTCCA | | | | | | | FGDHAMNWVRQAPG | | | | | | |
| | | GGGAAGGGGCTGGAGTGGGTAGGTTTCATTAGAACCAAAAC | | | | | | | KGLEWVGFIRTKTY | | | | | | |
| | | TTATGGTGGGACAACAGAATACGCCGCGTCTGTGAAAGGCA | | | | | | | GGTTEYAASVKGRF | | | | | | |
| | | GATTCACCATCTCAAGAGATGATTCCAAAAGCATCGCCTAT | | | | | | | TISRDDSKSIAYLQ | | | | | | |
| | | CTGCAAATGAACAGCCTGAGAACCGAGGACACAGCCGTGTA | | | | | | | MNSLRTEDTAVYYC | | | | | | |
| | | TTACTGTACTGGAGTCTTAGGTTGGCGGGGGAGTTATCGTG | | | | | | | TGVLGWRGSYREDY | | | | | | |
| | | AAGACTACTGGGGCCAGGGAACCCTGGTCACCGTCTCCTCA | | | | | | | WGQGTLVTVSS | | | | | | |
| ADI-81622 | 1291 | CAGGTGCAGCTGCAGCAGTCGGGCCCAGGACTGGTGAAGCC | | | | | | 6974 | QVQLQQSGPGLVKP | | 7325 | 7 | | 7 | |
| | | TTCGGAGACCCTGTCCCTCACCTGCACTGTCTCTGGTGGCT | | | | | | | SETLSLTCTVSGGS | | | | | | |
| | | CCATCAGCAGCAGGAGTTACTACTGGGACTGGATCCGCCAG | | | | | | | ISSRSYYWDWIRQS | | | | | | |
| | | TCCCCAGGGAGGGGGCTGGAGTGGATTGGGAGTATCTATTA | | | | | | | PGRGLEWIGSIYYS | | | | | | |
| | | TAGTGGGAGCGCCGACTACAACCCGTCCCTCAAGAGTCGAG | | | | | | | GSADYNPSLKSRVT | | | | | | |
| | | TCACCATATCCGTCGACACGTCCAAGAACCATTTCTCCCTG | | | | | | | ISVDTSKNHFSLKL | | | | | | |
| | | AAGCTGAGCTCTGTGACCGCCGCAGACACGGCTGTGTATTA | | | | | | | SSVTAADTAVYYCA | | | | | | |
| | | CTGTGCGAGGCGACCGACTACGGACTTCTACTACATGGACG | | | | | | | RRPTTDFYYMDVWG | | | | | | |
| | | TCTGGGGCAAAGGGACCACGGTCACCGTCTCCTCA | | | | | | | KGTTVTVSS | | | | | | |
| ADI-81644 | 1291 | CAGGTGCAGCTGTTGGAGTCGGGGGGAGGCTTGGTACAGCC | | | | | | 6975 | QVQLLESGGGLVQP | | 7326 | 7 | | 6 | |
| | | TGGCAGGTCCCTGCGACTCACCTGTGCAGCCTCTGGATTCA | | | | | | | GRSLRLTCAASGFT | | | | | | |
| | | CCTTTGATGATTATGCCATGCACTGGGTCCGGCAAGCTCCA | | | | | | | FDDYAMHWVRQAPG | | | | | | |
| | | GGGAAGGGCCTGGAGTGGGTCTCAGGTATTACTTGGAATAG | | | | | | | KGLEWVSGITWNSG | | | | | | |
| | | TGGTACCATAGGCTATGCGGACTCTGTGAAGGGCCGATTCA | | | | | | | TIGYADSVKGRFII | | | | | | |
| | | TCATTATCAGAGACAACGCCAAGAACTCCCTGTATCTCCAA | | | | | | | IRDNAKNSLYLQMN | | | | | | |
| | | ATGAACAGTCTGAGAGTTGAGGACACGGCCTTGTATTACTG | | | | | | | SLRVEDTALYYCVK | | | | | | |
| | | CGTAAAAGATAGCCGCGAAATCAAAACACTTATGGTTGAAG | | | | | | | DSREIKTLMVEEDY | | | | | | |
| | | AAGACTACTACATGGACGTCTGGGGCAAAGGGACCACGGTC | | | | | | | YMDVWGKGTTVTVS | | | | | | |
| | | ACCGTCTCCTCA | | | | | | | S | | | | | | |
| ADI-81858 | 1287 | GAGGTGCAGCTGGTGGAGTCTGGGGGAGGCGTGGTCCAGCC | | | | | | 6976 | EVQLVESGGGVVQP | | 7327 | 7 | | 7 | |
| | | TGGGAGGTCCCTGAGACTCTCCTGTGCAGCCTCTGGATTCA | | | | | | | GRSLRLSCAASGFT | | | | | | |
| | | CCTTCATTAACTATGGCATGCACTGGGTCCGCCAGGCTCCA | | | | | | | FINYGMHWVRQAPG | | | | | | |
| | | GGCAAGGGGCTGGAGTGGGTGGCAGCTATATCATATGATGG | | | | | | | KGLEWVAAISYDGS | | | | | | |
| | | AAGTGATGAATACTATGCAGACTCCGTGAAGGGCCGATTCA | | | | | | | DEYYADSVKGRFTI | | | | | | |
| | | CCATCTCCAGAGACGATTCCAAGAACACGGTGTATCTGCAA | | | | | | | SRDDSKNTVYLQMN | | | | | | |
| | | ATGAACAGCCTGAGAGCTGAGGACACGGCTGTGTATTACTG | | | | | | | SLRAEDTAVYYCAK | | | | | | |
| | | TGCGAAAGTGACCGCCTCCCATTATTACTACTACTACGGGA | | | | | | | VTASHYYYYYGMDV | | | | | | |
| | | TGGACGTCTGGGGCCAAGGGACCACGGTCACCGTCTCCTCA | | | | | | | WGQGTTVTVSS | | | | | | |
| ADI-81603 | 1291 | TGGGTCCTCGGTGAAGGTCTCCTGCAAGGCTTCTGGAGGCA | | | | | | 6977 | QVQLVQSGAEVKKP | | 7328 | 6 | | 5 | |
| | | CCTTCAGCAGCTATCCTATCAGCTGGGTGCGACAGGCCCCT | | | | | | | GSSVKVSCKASGGT | | | | | | |
| | | GGACAAGGGCTTGAGTGGATGGGAAGGATCATCCCTCCCCT | | | | | | | FSSYPISWVRQAPG | | | | | | |
| | | TGGTATATCAGACTACGCACAGAAGTTCCAGGGCAGAGTCA | | | | | | | QGLEWMGRIIPPLG | | | | | | |
| | | CAATAACCGCGGACAAATCCACGAGCACAGCCTACATGGAG | | | | | | | ISDYAQKFQGRVTI | | | | | | |
| | | CTGAGCAGCCTGACATCTGAGGACACGGCCGTGTATTACTG | | | | | | | TADKSTSTAYMELS | | | | | | |
| | | TGCGAGAGATCGAGGTTACTCCTCTGTTTCAGGGAGTAACT | | | | | | | SLTSEDTAVYYCAR | | | | | | |
| | | ACTACATGGACGTCTGGGGCAAAGGGACCACGGTCACCGTC | | | | | | | DRGYSSVSGSNYYM | | | | | | |
| | | TCCTCA | | | | | | | DVWGKGTTVTVSS | | | | | | |
| ADI-81535 | 1286 | TGGGTCCTCGGTGAAGGTCTCCTGCAAGGCTTCTGGAGGCA | | | | | | 6978 | QVQLVQSGAEVKKP | | 7329 | 5 | | 5 | |
| | | CCTTCAGCAGCAATCCTTTCAGCTGGGTGCGACAGGCCCCT | | | | | | | GSSVKVSCKASGGT | | | | | | |
| | | GGACAAGGGCTTGAGTGGATGGGAAGGATCATCCCTATCCT | | | | | | | FSSNPFSWVRQAPG | | | | | | |
| | | TGGTATACCAAACTACGCACAGAAGTTCCAGGGCAGAGTCA | | | | | | | QGLEWMGRIIPILG | | | | | | |
| | | CGTTTACCGCGGACAAATCCACGAGCACAGCCTACATGGAG | | | | | | | IPNYAQKFQGRVTF | | | | | | |
| | | TTGAGCAGCCTGAGATCTGAGGACACGGCCGTCTATTACTG | | | | | | | TADKSTSTAYMELS | | | | | | |
| | | TGCGAGAGATGCGGGTTACTCTGGTTCGGGGAGTGCTTACT | | | | | | | SLRSEDTAVYYCAR | | | | | | |
| | | ACTTTGACTACTGGGGCCAGGGAACCCTGGTCACCGTCTCC | | | | | | | DAGYSGSGSAYYFD | | | | | | |
| | | TCA | | | | | | | YWGQGTLVTVSS | | | | | | |
| ADI-81713 | 1287 | CAGGTCCAGCTTGTACAGTCTGGGCCTGAGGTGAAGAAGCC | | | | | | 6979 | QVQLVQSGPEVKKP | | 7330 | 5 | | 5 | |
| | | TGGGACCTCAGTGAAAGTCTCCTGCAAGGCTTCTGGATTCA | | | | | | | GTSVKVSCKASGFT | | | | | | |
| | | CCTTCATTACCTCTGCTGTGCAGTGGGTGCGACAGGCTCGT | | | | | | | FITSAVQWVRQARG | | | | | | |
| | | GGACAACGCCTTGAGTGGATAGGATGGATCGCCGTTGGCAG | | | | | | | QRLEWIGWIAVGRG | | | | | | |
| | | AGGTAACACAGACTACGCACAGAAGTTCCAGGAAAGAGTCA | | | | | | | NTDYAQKFQERVTI | | | | | | |
| | | CCATTACCAGGGACATGTCCACAAGCACAGCCTACATGGAG | | | | | | | TRDMSTSTAYMELS | | | | | | |
| | | CTGAGCAGCCTGAGATCCGAGGACACGGCCGTGTATTACTG | | | | | | | SLRSEDTAVYYCAA | | | | | | |
| | | TGCGGCCCTTAATTGTAATGTCACGACCTGCTCTGATGGTT | | | | | | | LNCNVTTCSDGFDM | | | | | | |
| | | TTGATATGTGGGGCCAAGGGACCACGGTCACCGTCTCCTCA | | | | | | | WGQGTTVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81816 | 90 | IGHV3- | EVQLL | 5417 | FTVSTN | 5628 | WVRQAP | 437 | VIYSGG | 6001 | RFTISR | 6311 | ARVVSDA | 6617 | WGQGTT |
| | | 53 | ESGGG | | YMS | | GKGLEW | | TTYYAD | | DNSKNT | | FDI | | VTVSS |
| | | | LIQRG | | | | VS | | SVKG | | LYLQMS | | | | |
| | | | GSLRL | | | | | | | | SLRAED | | | | |
| | | | SCAAS | | | | | | | | TAVYYC | | | | |
| | | | G | | | | | | | | | | | | |
| ADI-81895 | 70 | IGHV4- | EVQLL | 5346 | GSISSG | 5629 | WIRQHP | 5823 | YIYYSG | 675 | RVTISV | 1009 | ARTYYYD | 6618 | WGQGTL |
| | | 31 | ESGPG | | AYYWT | | GQGLEW | | ITYYNP | | DTSKNQ | | DNGNYYQ | | VTVSS |
| | | | LVKPS | | | | IG | | SLKS | | FSLKLS | | YYFDY | | |
| | | | QTLSL | | | | | | | | SVTAAD | | | | |
| | | | TCTVS | | | | | | | | TAVYYC | | | | |
| | | | G | | | | | | | | | | | | |
| ADI-81793 | 69 | IGHV3- | EVQLV | 5418 | VNLKEY | 5630 | WYRQAP | 5824 | TIS- | 6002 | RFTISR | 6312 | SIVSRDI | 6619 | WGRGTQ |
| | | 23 | ESGGG | | SMG | | GSARGA | | SGGAAF | | DNAKNE | | | | VTVSS |
| | | | FVQAG | | | | VA | | YSNAVT | | VYLQMN | | | | |
| | | | QSLRL | | | | | | G | | GLKDED | | | | |
| | | | SCTAS | | | | | | | | TGVYYC | | | | |
| | | | G | | | | | | | | | | | | |
| ADI-81724 | 1 | IGHV4- | EVQLL | 5346 | DSIASG | 5631 | WIRQHP | 5823 | NIYFTG | 6003 | RLSISV | 6313 | ARDGGSG | 6620 | WGQGIK |
| | | 31 | ESGPG | | GYYWT | | GQGLEW | | DTDYHP | | DTFRNR | | YHNLDY | | VTVSS |
| | | | LVKPS | | | | IG | | SLKS | | FSLKLT | | | | |
| | | | QTLSL | | | | | | | | SVTAAD | | | | |
| | | | TCTVS | | | | | | | | TAVYFC | | | | |
| | | | G | | | | | | | | | | | | |
| ADI-81599 | 59 | IGHV1- | QVQLV | 50 | GTFIRN | 306 | WVRQAP | 433 | RVIPLL | 637 | RVTIIA | 6314 | AREGTGY | 6621 | WGQGTT |
| | | 69 | QSGAE | | TIS | | GQGLEW | | NVVDYA | | DKSSST | | CDGGICP | | VTVSS |
| | | | VKKPG | | | | MG | | QKFQG | | VYMELT | | DAFDI | | |
| | | | SSVKV | | | | | | | | TLRSED | | | | |
| | | | SCKAS | | | | | | | | TAVYYC | | | | |
| | | | G | | | | | | | | | | | | |
| ADI-81705 | 75 | IGHV1- | RSSWC | 5419 | YTFTTY | 5632 | WVRQAP | 5825 | IINPDG | 6004 | RVTMTT | 6315 | ARGSLIP | 6622 | WGRGTT |
| | | 46 | SLGAE | | YIH | | GEGLEW | | GAITYA | | DTSTST | | AKDGFDI | | VTVSS |
| | | | VKKPG | | | | MG | | QKFRG | | VYMNVI | | | | |
| | | | ASVKV | | | | | | | | SLRSED | | | | |
| | | | SCKAS | | | | | | | | TAVYYC | | | | |
| | | | G | | | | | | | | | | | | |
| ADI-81760 | 36 | IGHV1- | EVQLV | 5320 | YTFTTY | 5632 | WVRQAP | 433 | IINPDG | 6005 | RLTMTT | 6316 | ARDLVAI | 6623 | WGQGTL |
| | | 46 | QSGAE | | YIH | | GQGLEW | | GARTYA | | DTSTTT | | PAAMCGA | | VTVSS |
| | | | VKKPG | | | | MG | | QHFQG | | VYMELS | | Y | | |
| | | | ASVKV | | | | | | | | NLGSED | | | | |
| | | | SCKAS | | | | | | | | TAMYYC | | | | |
| | | | G | | | | | | | | | | | | |
| ADI-81843 | 21 | IGHV3- | EVQLV | 5420 | FTFSRY | 5633 | WVRQAP | 431 | GISFDG | 6006 | RFTISR | 6317 | AKGLGTY | 6624 | WGQGTL |
| | | 30 | ESGGG | | GMH | | GKGLEW | | DDKDYA | | DNSKDT | | CSGGNCY | | VTVSS |
| | | | VVQPG | | | | VA | | DSLKG | | LYLQMN | | SHYFDY | | |
| | | | RSLRL | | | | | | | | GLSTED | | | | |
| | | | SCTAS | | | | | | | | TAVYFC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81816 | 1287 | GAGGTGCAGCTGTTGGAGTCTGGAGGAGGCTTGATCCAGCG | | | | | | 6980 | EVQLLESGGGLIQR | | 7331 | 5 | | 4 | |
| | | GGGGGGGTCCCTGAGACTCTCCTGTGCAGCCTCTGGATTCA | | | | | | | GGSLRLSCAASGFT | | | | | | |
| | | CCGTCAGTACCAACTACATGAGCTGGGTCCGCCAGGCTCCA | | | | | | | VSTNYMSWVRQAPG | | | | | | |
| | | GGGAAGGGGCTGGAGTGGGTCTCAGTTATTTATAGCGGTGG | | | | | | | KGLEWVSVIYSGGT | | | | | | |
| | | TACCACATACTACGCAGACTCCGTGAAGGGCCGATTCACCA | | | | | | | TYYADSVKGRFTIS | | | | | | |
| | | TCTCCAGAGACAATTCCAAGAACACGCTGTATCTTCAAATG | | | | | | | RDNSKNTLYLQMSS | | | | | | |
| | | AGCAGCCTGAGAGCCGAGGACACGGCCGTGTATTACTGTGC | | | | | | | LRAEDTAVYYCARV | | | | | | |
| | | GAGAGTCGTGTCGGATGCTTTTGATATCTGGGGCCAAGGGA | | | | | | | VSDAFDIWGQGTTV | | | | | | |
| | | CCACGGTCACCGTCTCCTCA | | | | | | | TVSS | | | | | | |
| ADI-81895 | 1286 | GAGGTGCAGCTGTTGGAGTCGGGCCCAGGACTGGTGAAGCC | | | | | | 6981 | EVQLLESGPGLVKP | | 7332 | 5 | | 5 | |
| | | TTCACAGACCCTGTCCCTCACCTGCACTGTCTCTGGTGGCT | | | | | | | SQTLSLTCTVSGGS | | | | | | |
| | | CCATCAGCAGTGGTGCTTACTACTGGACCTGGATCCGCCAG | | | | | | | ISSGAYYWTWIRQH | | | | | | |
| | | CACCCAGGGCAGGGCCTGGAGTGGATTGGGTACATCTATTA | | | | | | | PGQGLEWIGYIYYS | | | | | | |
| | | CAGTGGGATCACCTACTACAATCCGTCCCTCAAGAGTCGAG | | | | | | | GITYYNPSLKSRVT | | | | | | |
| | | TCACCATATCAGTAGACACGTCTAAGAACCAGTTCTCCCTG | | | | | | | ISVDTSKNQFSLKL | | | | | | |
| | | AAGCTGAGCTCTGTGACTGCCGCGGACACGGCCGTTTATTA | | | | | | | SSVTAADTAVYYCA | | | | | | |
| | | CTGTGCGAGGACGTATTACTATGATGATAATGGTAATTACT | | | | | | | RTYYYDDNGNYYQY | | | | | | |
| | | ACCAGTACTACTTTGACTACTGGGGCCAGGGAACCCTGGTC | | | | | | | YFDYWGQGTLVTVS | | | | | | |
| | | ACCGTCTCCTCA | | | | | | | S | | | | | | |
| ADI-81793 | 6814 | GAGGTGCAGCTCGTGGAGTCTGGGGGAGGCTTTGTACAGGC | | | | | | 6982 | EVQLVESGGGFVQA | | 7333 | 54 | | 34 | |
| | | TGGGCAGTCTCTGAGACTCTCCTGTACAGCCTCTGGAGTCA | | | | | | | GQSLRLSCTASGVN | | | | | | |
| | | ACCTCAAAGAGTATAGTATGGGCTGGTACCGCCAGGCTCCA | | | | | | | LKEYSMGWYRQAPG | | | | | | |
| | | GGGAGCGCGCGCGGAGCGGTCGCAACTATCAGTAGTGGTGG | | | | | | | SARGAVATISSGGA | | | | | | |
| | | TGCTGCATTCTATTCAAATGCCGTGACGGGCCGATTCACCA | | | | | | | AFYSNAVTGRFTIS | | | | | | |
| | | TCTCCAGAGACAACGCCAAGAACGAAGTGTATCTGCAGATG | | | | | | | RDNAKNEVYLQMNG | | | | | | |
| | | AACGGCCTGAAAGATGAGGACACGGGCGTCTATTACTGTAG | | | | | | | LKDEDTGVYYCSIV | | | | | | |
| | | CATCGTGTCGCGTGACATATGGGGCCGGGGAACCCAGGTCA | | | | | | | SRDIWGRGTQVTVS | | | | | | |
| | | CCGTCTCTTCA | | | | | | | S | | | | | | |
| ADI-81724 | 6815 | GAGGTGCAGCTGTTGGAGTCGGGCCCAGGACTGGTGAAGCC | | | | | | 6983 | EVQLLESGPGLVKP | | 7334 | 21 | | 18 | |
| | | TTCACAGACCCTGTCCCTCACCTGCACTGTCTCTGGTGACT | | | | | | | SQTLSLTCTVSGDS | | | | | | |
| | | CCATCGCCAGTGGTGGTTATTACTGGACCTGGATCCGCCAG | | | | | | | IASGGYYWTWIRQH | | | | | | |
| | | CACCCCGGGCAGGGCCTGGAGTGGATTGGGAACATCTATTT | | | | | | | PGQGLEWIGNIYFT | | | | | | |
| | | CACTGGGGACACCGACTACCACCCGTCCCTCAAGAGTCGAC | | | | | | | GDTDYHPSLKSRLS | | | | | | |
| | | TTAGCATATCAGTAGACACGTTTAGGAACCGGTTCTCCCTG | | | | | | | ISVDTFRNRFSLKL | | | | | | |
| | | AAGTTGACCTCTGTGACTGCCGCGGACACGGCCGTATATTT | | | | | | | TSVTAADTAVYFCA | | | | | | |
| | | TTGTGCGAGAGACGGCGGTAGTGGCTACCACAACCTTGACT | | | | | | | RDGGSGYHNLDYWG | | | | | | |
| | | ATTGGGGCCAGGGAATCAAGGTCACCGTCTCCTCA | | | | | | | QGIKVTVSS | | | | | | |
| ADI-81599 | 1287 | TGGGTCCTCGGTGAAGGTCTCCTGCAAGGCTTCTGGAGGCA | | | | | | 6984 | QVQLVQSGAEVKKP | | 7335 | 19 | | 14 | |
| | | CCTTCATCAGGAACACTATAAGTTGGGTGCGACAGGCCCCT | | | | | | | GSSVKVSCKASGGT | | | | | | |
| | | GGACAGGGGCTTGAGTGGATGGGAAGGGTCATCCCTCTACT | | | | | | | FIRNTISWVRQAPG | | | | | | |
| | | TAATGTAGTTGACTACGCACAGAAGTTCCAGGGCAGAGTCA | | | | | | | QGLEWMGRVIPLLN | | | | | | |
| | | CGATAATCGCGGACAAATCTTCGAGCACAGTCTACATGGAG | | | | | | | VVDYAQKFQGRVTI | | | | | | |
| | | TTGACTACCCTGAGATCTGAGGACACGGCCGTCTATTACTG | | | | | | | IADKSSSTVYMELT | | | | | | |
| | | TGCGAGAGAGGGGACCGGCTATTGTGATGGTGGTATATGCC | | | | | | | TLRSEDTAVYYCAR | | | | | | |
| | | CTGATGCTTTTGATATCTGGGGCCAAGGGACCACGGTCACC | | | | | | | EGTGYCDGGICPDA | | | | | | |
| | | GTCTCCTCA | | | | | | | FDIWGQGTTVTVSS | | | | | | |
| ADI-81705 | 1294 | AGGTCCAGCTGGTGCAGTCTGGGCGCTGAGGTGAAGAAGCC | | | | | | 6985 | RSSWCSLGAEVKKP | | 7336 | 17 | | 12 | |
| | | TGGGGCCTCAGTGAAGGTTTCCTGCAAGGCATCTGGATACA | | | | | | | GASVKVSCKASGYT | | | | | | |
| | | CCTTCACCACCTACTATATTCACTGGGTGCGACAGGCCCCT | | | | | | | FTTYYIHWVRQAPG | | | | | | |
| | | GGAGAAGGGCTTGAGTGGATGGGAATAATCAACCCTGATGG | | | | | | | EGLEWMGIINPDGG | | | | | | |
| | | TGGTGCCATAACTTACGCACAGAAGTTCCGGGGCAGAGTCA | | | | | | | AITYAQKFRGRVTM | | | | | | |
| | | CCATGACCACGGACACGTCCACGAGTACAGTCTACATGAAT | | | | | | | TTDTSTSTVYMNVI | | | | | | |
| | | GTAATCAGCCTGAGATCTGAAGACACGGCCGTATATTACTG | | | | | | | SLRSEDTAVYYCAR | | | | | | |
| | | TGCGAGAGGGTCATTAATCCCAGCAAAGGATGGTTTTGATA | | | | | | | GSLIPAKDGFDIWG | | | | | | |
| | | TCTGGGGCCGAGGGACCACGGTCACCGTCTCCTCA | | | | | | | RGTTVTVSS | | | | | | |
| ADI-81760 | 1286 | GAGGTGCAGCTGGTGCAGTCTGGGGCTGAGGTGAAGAAGCC | | | | | | 6986 | EVQLVQSGAEVKKP | | 7337 | 17 | | 13 | |
| | | TGGGGCCTCAGTGAAGGTTTCCTGCAAGGCATCTGGATACA | | | | | | | GASVKVSCKASGYT | | | | | | |
| | | CCTTTACCACCTACTATATTCACTGGGTGCGACAGGCCCCT | | | | | | | FTTYYIHWVRQAPG | | | | | | |
| | | GGACAAGGGCTTGAGTGGATGGGAATAATCAACCCTGATGG | | | | | | | QGLEWMGIINPDGG | | | | | | |
| | | TGGTGCCAGAACCTACGCACAGCACTTCCAGGGCAGACTCA | | | | | | | ARTYAQHFQGRLTM | | | | | | |
| | | CCATGACCACGGACACGTCCACGACCACAGTCTACATGGAA | | | | | | | TTDTSTTTVYMELS | | | | | | |
| | | CTGAGCAACCTGGGCTCTGAGGACACGGCCATGTATTACTG | | | | | | | NLGSEDTAMYYCAR | | | | | | |
| | | TGCGAGAGATCTTGTCGCGATACCAGCTGCTATGTGTGGCG | | | | | | | DLVAIPAAMCGAYW | | | | | | |
| | | CCTACTGGGGCCAGGGAACCCTGGTCACCGTCTCCTCA | | | | | | | GQGTLVTVSS | | | | | | |
| ADI-81843 | 1286 | GAGGTGCAGCTGGTGGAGTCTGGGGGAGGCGTGGTCCAGCC | | | | | | 6987 | EVQLVESGGGVVQP | | 7338 | 16 | | 13 | |
| | | TGGGAGGTCCCTGAGACTCTCCTGTACAGCCTCTGGATTCA | | | | | | | GRSLRLSCTASGFT | | | | | | |
| | | CCTTCAGTCGTTATGGCATGCACTGGGTCCGCCAGGCTCCA | | | | | | | FSRYGMHWVRQAPG | | | | | | |
| | | GGCAAGGGGCTGGAGTGGGTGGCAGGTATATCGTTTGATGG | | | | | | | KGLEWVAGISFDGD | | | | | | |
| | | AGATGACAAAGACTATGCAGACTCCCTGAAGGGCCGATTCA | | | | | | | DKDYADSLKGRFTI | | | | | | |
| | | CCATCTCCAGAGACAATTCCAAGGACACGCTGTATCTCCAA | | | | | | | SRDNSKDTLYLQMN | | | | | | |
| | | ATGAACGGCCTGAGCACTGAGGACACGGCTGTGTATTTCTG | | | | | | | GLSTEDTAVYFCAK | | | | | | |
| | | TGCGAAGGGTCTGGGGACGTATTGTAGTGGTGGCAACTGCT | | | | | | | GLGTYCSGGNCYSH | | | | | | |
| | | ACTCTCACTACTTTGACTACTGGGGCCAGGGAACCCTGGTC | | | | | | | YFDYWGQGTLVTVS | | | | | | |
| | | ACCGTCTCCTCA | | | | | | | S | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81887 | 63 | IGHV3- | EVQLV | 5421 | VSFSSS | 5634 | WVRQAP | 431 | VISNDG | 6007 | RFTISR | 6318 | ANDYGDL | 6625 | WGQGAL |
| | | 30 | ESGGG | | GMH | | GKGLEW | | NYKSYS | | DPSKNT | | VTWFDY | | VTVSS |
| | | | VVQPG | | | | VA | | DSVKG | | LYLQMN | | | | |
| | | | RSLRL | | | | | | | | SLRGED | | | | |
| | | | ACTAS | | | | | | | | TAVYYC | | | | |
| | | | G | | | | | | | | | | | | |
| ADI-81580 | 41 | IGHV1- | RYQLV | 5422 | GTF- | 364 | WVRQAP | 433 | RIIPIL | 6008 | RVTITA | 6319 | AREGGST | 6626 | WGKGTL |
| | | 69 | QSGAE | | SYNIN | | GQGLEW | | GIPHYA | | DKSTST | | TAYFPH | | VTVSS |
| | | | VKKPG | | | | MG | | QTFQD | | LYMELS | | | | |
| | | | SSVKI | | | | | | | | SLRSED | | | | |
| | | | SCQAS | | | | | | | | TAIYYC | | | | |
| | | | G | | | | | | | | | | | | |
| ADI-81591 | 51 | IGHV1- | QVQLV | 5423 | YTFISQ | 5635 | WVRQAP | 433 | IINPDA | 6009 | RLTMTT | 6320 | TRADDFI | 6627 | WGRGTM |
| | | 46 | QSGAE | | YIH | | GQGLEW | | GSTTYA | | DTSTST | | PSPFGAF | | VTVSS |
| | | | VKKPG | | | | MG | | QQFQG | | VYMELR | | EI | | |
| | | | ASVQV | | | | | | | | SLRSED | | | | |
| | | | SCKAS | | | | | | | | TAVYYC | | | | |
| | | | G | | | | | | | | | | | | |
| ADI-81731 | 8 | IGHV1- | EVQLV | 5424 | GSFSNF | 5636 | WVRQAP | 433 | GIIPLF | 6010 | RVTVTA | 6321 | ARGSLYH | 6628 | RGNGTT |
| | | 69 | ESGSE | | AFN | | GQGLEW | | GAPNYA | | DKSTGT | | DSSRGSL | | VTVSS |
| | | | VKKPG | | | | MG | | QKFQG | | AHMELS | | DNHYYME | | |
| | | | SSVKV | | | | | | | | SLRSED | | V | | |
| | | | SCKAS | | | | | | | | TAIYYC | | | | |
| | | | G | | | | | | | | | | | | |
| ADI-81750 | 26 | IGHV3- | EVQLV | 5425 | VTFDGS | 5637 | WVRQVP | 5826 | LISGHG | 6011 | RFTISR | 6322 | VKDAVLV | 6629 | WGRGTL |
| | | 43 | ESGGG | | AMH | | GRGLEW | | GSTYYA | | DNSANS | | VYGMGNW | | VTVSS |
| | | | VVQPG | | | | VS | | ESVKG | | LYLQMN | | YFDL | | |
| | | | GSLRL | | | | | | | | SLSTED | | | | |
| | | | SCLGY | | | | | | | | TAFYYC | | | | |
| | | | G | | | | | | | | | | | | |
| ADI-81830 | 9 | IGHV4- | QVQLQ | 5426 | GSFNTF | 5638 | WIRQPA | 5827 | DINHSG | 6012 | RLTISV | 6323 | ARGRPGW | 6630 | WGQGTL |
| | | 34 | QWGAG | | FWT | | GRGLEW | | NTNYNP | | DTSKNQ | | ELIPFFD | | VTVSS |
| | | | LLKPS | | | | IG | | SLKS | | FSLKLS | | S | | |
| | | | ENLSL | | | | | | | | SVTAAD | | | | |
| | | | TCGVS | | | | | | | | TAVYYC | | | | |
| | | | G | | | | | | | | | | | | |
| ADI-81774 | 50 | IGHV1- | EVQLV | 5402 | GSFSTF | 5639 | WVRQAP | 5828 | RISPSQ | 6013 | RVSITA | 6324 | ARDLGAG | 6631 | WGQGTL |
| | | 69 | ESGAE | | AIS | | GQGLEW | | GISNYA | | DKSTST | | SYYRHFD | | VTVSS |
| | | | VKKPG | | | | VG | | QRFQG | | ASMELS | | S | | |
| | | | SSVKV | | | | | | | | SLRSED | | | | |
| | | | SCKAS | | | | | | | | TAVYFC | | | | |
| | | | G | | | | | | | | | | | | |
| ADI-81804 | 80 | IGHV1- | QVQLV | 5427 | YTFSNY | 5640 | WVRQAP | 433 | LINPRG | 6014 | RVTMSR | 6325 | ARDPGVV | 6632 | WGQGTL |
| | | 46 | QSGVE | | YIL | | GQGLEW | | GGTSYA | | DTSTST | | DPLSPHP | | VTVSS |
| | | | VKKPG | | | | MG | | HKFQG | | AYMELT | | LPRF | | |
| | | | ASVML | | | | | | | | SLRSED | | | | |
| | | | SCKAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81887 | 1285 | GAGGTGCAGCTGGTGGAGTCTGGGGGAGGCGTGGTCCAGCC | | | | | | 6988 | EVQLVESGGGVVQP | | 7339 | 16 | | 12 | |
| | | TGGGAGGTCCCTGAGACTCGCCTGCACAGCCTCTGGAGTCT | | | | | | | GRSLRLACTASGVS | | | | | | |
| | | CCTTCAGTAGTTCTGGCATGCACTGGGTCCGCCAGGCTCCA | | | | | | | FSSSGMHWVRQAPG | | | | | | |
| | | GGCAAGGGGCTGGAGTGGGTGGCAGTTATATCAAATGATGG | | | | | | | KGLEWVAVISNDGN | | | | | | |
| | | AAATTACAAATCTTATTCAGACTCCGTGAAGGGCCGATTCA | | | | | | | YKSYSDSVKGRFTI | | | | | | |
| | | CCATCTCCAGAGACCCTTCCAAGAACACACTGTATCTGCAA | | | | | | | SRDPSKNTLYLQMN | | | | | | |
| | | ATGAACAGCCTGAGAGGAGAGGACACGGCTGTGTATTATTG | | | | | | | SLRGEDTAVYYCAN | | | | | | |
| | | TGCAAACGACTACGGTGACTTAGTAACCTGGTTTGACTACT | | | | | | | DYGDLVTWFDYWGQ | | | | | | |
| | | GGGGCCAGGGAGCCCTGGTCACCGTCTCCTCA | | | | | | | GALVTVSS | | | | | | |
| ADI-81580 | 6816 | AGGTACCAGCTGGTGCAGTCTGGGGCTGAGGTGAAGAAGCC | | | | | | 6989 | RYQLVQSGAEVKKP | | 7340 | 15 | | 11 | |
| | | TGGGTCCTCGGTGAAAATCTCCTGCCAGGCTTCTGGAGGCA | | | | | | | GSSVKISCQASGGT | | | | | | |
| | | CTTTCAGTTATAATATAAACTGGGTGCGACAGGCCCCTGGA | | | | | | | FSYNINWVRQAPGQ | | | | | | |
| | | CAAGGACTTGAGTGGATGGGAAGGATCATCCCTATCCTTGG | | | | | | | GLEWMGRIIPILGI | | | | | | |
| | | AATACCACACTACGCACAGACGTTTCAGGACAGAGTCACGA | | | | | | | PHYAQTFQDRVTIT | | | | | | |
| | | TAACCGCGGACAAATCCACGAGCACCCTCTACATGGAGCTG | | | | | | | ADKSTSTLYMELSS | | | | | | |
| | | AGCAGCCTGAGATCTGAGGACACGGCCATATATTACTGTGC | | | | | | | LRSEDTAIYYCARE | | | | | | |
| | | GAGAGAAGGTGGTTCCACCACTGCATACTTCCCGCACTGGG | | | | | | | GGSTTAYFPHWGKG | | | | | | |
| | | GCAAGGGCACCCTGGTCACCGTCTCCTCA | | | | | | | TLVTVSS | | | | | | |
| ADI-81591 | 6817 | CAGGTGCAGCTGGTGCAGTCTGGGGCTGAGGTGAAGAAGCC | | | | | | 6990 | QVQLVQSGAEVKKP | | 7341 | 15 | | 11 | |
| | | TGGGGCCTCAGTGCAGGTTTCCTGCAAGGCATCTGGATACA | | | | | | | GASVQVSCKASGYT | | | | | | |
| | | CCTTCATTAGCCAGTATATACACTGGGTGCGACAGGCCCCT | | | | | | | FISQYIHWVRQAPG | | | | | | |
| | | GGACAAGGCCTAGAGTGGATGGGAATAATCAACCCTGACGC | | | | | | | QGLEWMGIINPDAG | | | | | | |
| | | TGGTAGCACAACCTACGCACAGCAGTTCCAGGGCAGACTCA | | | | | | | STTYAQQFQGRLTM | | | | | | |
| | | CCATGACCACGGACACGTCCACGAGCACAGTGTACATGGAA | | | | | | | TTDTSTSTVYMELR | | | | | | |
| | | CTGAGGAGCCTGAGATCTGAGGACACGGCCGTTTATTACTG | | | | | | | SLRSEDTAVYYCTR | | | | | | |
| | | TACTAGAGCCGACGATTTTATTCCCTCGCCTTTCGGTGCTT | | | | | | | ADDFIPSPFGAFEI | | | | | | |
| | | TTGAGATCTGGGGCCGAGGAACAATGGTCACCGTCTCCTCA | | | | | | | WGRGTMVTVSS | | | | | | |
| ADI-81731 | 6818 | GAGGTGCAGCTGGTGGAGTCTGGGTCTGAGGTGAAGAAGCC | | | | | | 6991 | EVQLVESGSEVKKP | | 7342 | 15 | | 12 | |
| | | TGGGTCCTCGGTGAAGGTCTCCTGCAAGGCTTCTGGAGGCA | | | | | | | GSSVKVSCKASGGS | | | | | | |
| | | GTTTCAGCAACTTTGCTTTCAACTGGGTGCGACAGGCCCCT | | | | | | | FSNFAFNWVRQAPG | | | | | | |
| | | GGACAAGGCCTTGAGTGGATGGGAGGGATCATCCCTCTCTT | | | | | | | QGLEWMGGIIPLFG | | | | | | |
| | | TGGTGCACCAAACTACGCACAGAAGTTCCAGGGCAGAGTCA | | | | | | | APNYAQKFQGRVTV | | | | | | |
| | | CTGTAACCGCGGACAAATCCACGGGCACTGCCCACATGGAG | | | | | | | TADKSTGTAHMELS | | | | | | |
| | | TTGAGCAGCCTGAGATCTGAAGACACGGCCATATATTACTG | | | | | | | SLRSEDTAIYYCAR | | | | | | |
| | | TGCGAGGGGCTCTCTTTATCATGATTCCAGCCGGGGAAGTT | | | | | | | GSLYHDSSRGSLDN | | | | | | |
| | | TGGACAACCATTATTACATGGAGGTCCGGGGCAATGGGACC | | | | | | | HYYMEVRGNGTTVT | | | | | | |
| | | ACGGTCACCGTCTCCTCA | | | | | | | VSS | | | | | | |
| ADI-81750 | 1288 | TGGGGGGTCCCTGAGACTCTCCTGTCTAGGCTATGGAGTCA | | | | | | 6992 | EVQLVESGGGVVQP | | 7343 | 15 | | 13 | |
| | | CCTTTGATGGTTCTGCCATGCACTGGGTCCGTCAAGTTCCA | | | | | | | GGSLRLSCLGYGVT | | | | | | |
| | | GGGAGGGGTCTGGAGTGGGTCTCCCTTATTAGTGGGCATGG | | | | | | | FDGSAMHWVRQVPG | | | | | | |
| | | TGGTAGCACATACTATGCAGAGTCTGTGAAGGGCCGATTCA | | | | | | | RGLEWVSLISGHGG | | | | | | |
| | | CCATCTCCAGAGACAACAGCGCAAACTCTCTATATCTCCAA | | | | | | | STYYAESVKGRFTI | | | | | | |
| | | ATGAACAGTCTGAGCACTGAGGACACCGCCTTCTACTACTG | | | | | | | SRDNSANSLYLQMN | | | | | | |
| | | TGTAAAAGACGCCGTGTTAGTGGTGTATGGCATGGGGAACT | | | | | | | SLSTEDTAFYYCVK | | | | | | |
| | | GGTACTTCGATCTCTGGGGCCGTGGCACCCTGGTCACCGTC | | | | | | | DAVLVVYGMGNWYF | | | | | | |
| | | TCCTCA | | | | | | | DLWGRGTLVTVSS | | | | | | |
| ADI-81830 | 1286 | CAGGTGCAGCTACAGCAGTGGGGCGCAGGGCTGTTGAAGCC | | | | | | 6993 | QVQLQQWGAGLLKP | | 7344 | 15 | | 13 | |
| | | TTCGGAGAACCTGTCCCTCACCTGCGGTGTCTCTGGTGGTT | | | | | | | SENLSLTCGVSGGS | | | | | | |
| | | CCTTCAACACTTTCTTCTGGACCTGGATCCGCCAGCCCGCA | | | | | | | FNTFFWTWIRQPAG | | | | | | |
| | | GGGAGGGGGCTGGAGTGGATTGGGGACATCAATCATAGTGG | | | | | | | RGLEWIGDINHSGN | | | | | | |
| | | AAACACCAACTACAACCCGTCCCTCAAGAGTCGACTCACCA | | | | | | | TNYNPSLKSRLTIS | | | | | | |
| | | TTTCAGTAGACACGTCCAAGAACCAGTTCTCCCTGAAACTG | | | | | | | VDTSKNQFSLKLSS | | | | | | |
| | | AGTTCTGTGACCGCCGCGGACACGGCTGTGTATTACTGTGC | | | | | | | VTAADTAVYYCARG | | | | | | |
| | | GAGAGGCAGGCCGGGGTGGGAGCTCATCCCCTTCTTTGACT | | | | | | | RPGWELIPFFDSWG | | | | | | |
| | | CCTGGGGCCAGGGAACCCTGGTCACCGTCTCCTCA | | | | | | | QGTLVTVSS | | | | | | |
| ADI-81774 | 1286 | GAGGTGCAGCTGGTGGAGTCTGGGGCTGAAGTGAAGAAGCC | | | | | | 6994 | EVQLVESGAEVKKP | | 7345 | 14 | | 12 | |
| | | TGGGTCCTCGGTGAAAGTCTCCTGCAAGGCTTCTGGAGGCA | | | | | | | GSSVKVSCKASGGS | | | | | | |
| | | GCTTCAGCACATTTGCCATCAGCTGGGTGCGACAGGCCCCT | | | | | | | FSTFAISWVRQAPG | | | | | | |
| | | GGACAAGGGCTTGAGTGGGTGGGAAGGATAAGCCCCAGCCA | | | | | | | QGLEWVGRISPSQG | | | | | | |
| | | GGGTATATCAAACTACGCACAGAGGTTCCAGGGCAGAGTCT | | | | | | | ISNYAQRFQGRVSI | | | | | | |
| | | CGATTACCGCGGACAAATCCACGAGCACAGCCTCCATGGAA | | | | | | | TADKSTSTASMELS | | | | | | |
| | | CTCAGCAGCCTGAGATCTGAGGACACGGCCGTCTATTTCTG | | | | | | | SLRSEDTAVYFCAR | | | | | | |
| | | TGCGCGAGATCTAGGTGCGGGGAGTTATTATCGACACTTTG | | | | | | | DLGAGSYYRHFDSW | | | | | | |
| | | ACTCCTGGGGCCAGGGAACCCTGGTCACCGTCTCCTCA | | | | | | | GQGTLVTVSS | | | | | | |
| ADI-81804 | 1286 | TGGGGCCTCAGTGATGCTTTCCTGCAAGGCATCTGGATACA | | | | | | 6995 | QVQLVQSGVEVKKP | | 7346 | 14 | | 13 | |
| | | CCTTCAGCAACTACTATATACTCTGGGTGCGACAGGCCCCT | | | | | | | GASVMLSCKASGYT | | | | | | |
| | | GGACAAGGGCTTGAGTGGATGGGACTAATCAACCCGCGTGG | | | | | | | FSNYYILWVRQAPG | | | | | | |
| | | TGGTGGCACAAGCTACGCACACAAGTTCCAGGGTAGAGTCA | | | | | | | QGLEWMGLINPRGG | | | | | | |
| | | CCATGAGTAGGGACACGTCCACGAGCACTGCCTACATGGAG | | | | | | | GTSYAHKFQGRVTM | | | | | | |
| | | TTGACCAGCCTGAGATCTGAGGACACGGCCGTGTATTACTG | | | | | | | SRDTSTSTAYMELT | | | | | | |
| | | TGCGAGAGATCCGGGTGTCGTGGACCCGCTGAGCCCCCACC | | | | | | | SLRSEDTAVYYCAR | | | | | | |
| | | CCCTGCCGCGGTTCTGGGGCCAGGGAACCCTGGTCACCGTC | | | | | | | DPGVVDPLSPHPLP | | | | | | |
| | | TCCTCA | | | | | | | RFWGQGTLVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81544 | 9 | IGHV1- | QVQLV | 50 | GPFGSS | 5641 | WVRQAP | 433 | RIIPIL | 739 | RVTITA | 6326 | ARDVGYS | 6633 | WGQGTL |
| | | 69 | QSGAE | | PIS | | GQGLEW | | GVADYA | | DRSTST | | GSGSSYY | | VTVSS |
| | | | VKKPG | | | | MG | | QKFQG | | TYMELS | | FDP | | |
| | | | SSVKV | | | | | | | | SLRSED | | | | |
| | | | SCKAS | | | | | | | | TARYYC | | | | |
| | | | G | | | | | | | | | | | | |
| ADI-81691 | 61 | IGHV3- | EVQLV | 5333 | FTLSND | 5642 | WVRQAP | 437 | AIGSAG | 6015 | RFTISR | 6327 | VRGTMTT | 6634 | WGRGTT |
| | | 13 | ESGGG | | DIH | | GKGLEW | | DTYYAD | | EKANNS | | LGVFQYY | | VTVSS |
| | | | LVQPG | | | | VS | | SEKG | | LYLQMH | | YKEI | | |
| | | | GSLRL | | | | | | | | SLRAGD | | | | |
| | | | SCVAS | | | | | | | | TAVYYC | | | | |
| | | | G | | | | | | | | | | | | |
| ADI-81571 | 34 | IGHV1- | QVQLQ | 86 | GSFNSH | 5643 | WVRQAP | 433 | RIIPIL | 739 | RVNIIA | 6328 | ARDVGYS | 6635 | WGQGTL |
| | | 69 | QSGAE | | SIS | | GQGLEW | | GVADYA | | DKSTGT | | GSGSAYY | | VTVSS |
| | | | VKKPG | | | | MG | | QKFQG | | AYMELR | | FDL | | |
| | | | SSVKV | | | | | | | | SLRSED | | | | |
| | | | SCKAS | | | | | | | | TAVYYC | | | | |
| | | | G | | | | | | | | | | | | |
| ADI-81850 | 28 | IGHV3- | EVQLV | 5428 | FTFSTY | 5644 | WVRQAP | 431 | VISDDG | 6016 | RFTISR | 6329 | TREGPMT | 6636 | WGQGTL |
| | | 30 | ESGGG | | AMH | | GKGLEW | | RYKDSA | | DNSQNT | | TVTYFDY | | VTVSS |
| | | | VVQPG | | | | VA | | DSVKG | | LYLQMS | | | | |
| | | | RSLRL | | | | | | | | SLRPED | | | | |
| | | | SCAAF | | | | | | | | TAIYYC | | | | |
| | | | G | | | | | | | | | | | | |
| ADI-81628 | 1 | IGHV4- | EVQLQ | 5429 | GSFSGN | 5645 | WIRQSP | 434 | EINHSG | 6017 | RVTISI | 6330 | AREVVVI | 6637 | WGAGTT |
| | | 34 | QSGAG | | YWS | | GKGLEW | | TTTYNP | | DTSKNQ | | PAATIRY | | VTVSS |
| | | | LLKPS | | | | IG | | SLKS | | FSLRLS | | SYNYYMD | | |
| | | | ETLSL | | | | | | | | SVTAAD | | | | |
| | | | TCGVF | | | | | | | | TAVYYC | | | | |
| | | | G | | | | | | | | | | | | |
| ADI-81678 | 48 | IGHV5- | EVQLL | 5430 | YSFTTY | 5646 | WVRQMP | 476 | RIDPSD | 6018 | RVSISA | 6331 | ARGRGSA | 6638 | WGQGTL |
| | | 10-1 | ESGAE | | WIT | | GKGLEW | | SYTKYS | | DKSTSN | | YDIMEIE | | VTAPQ |
| | | | VKKPG | | | | MG | | PSFQG | | AYMQWS | | Y | | |
| | | | ESLRI | | | | | | | | SLQASD | | | | |
| | | | SCKGS | | | | | | | | TAIYYC | | | | |
| | | | G | | | | | | | | | | | | |
| ADI-81738 | 15 | IGHV5- | QVQLV | 5431 | YTFSTY | 5619 | WVRQMP | 476 | IIFPGD | 5987 | QVTISA | 6332 | ATLKLEG | 6603 | WGQGTL |
| | | 51 | QSGAE | | WIG | | GKGLEW | | SDTRYS | | DKSIST | | FTPVDYN | | VTVSS |
| | | | VRKPG | | | | MG | | PSFQG | | AYVQWS | | TLPALY | | |
| | | | ESLKI | | | | | | | | SLKASD | | | | |
| | | | SCKAS | | | | | | | | TAMYFC | | | | |
| | | | G | | | | | | | | | | | | |
| ADI-81667 | 38 | IGHV3- | QVQLV | 5432 | FTVSNN | 5647 | WVRQPP | 5829 | FIYSGG | 6019 | RFTITR | 6333 | ATSPAVG | 6639 | WGQGTM |
| | | 66 | ESGGD | | YMS | | GKGLEW | | NTFYAD | | DNSKNT | | V | | VTVSS |
| | | | LVQPG | | | | VS | | SVKG | | LWLQMN | | | | |
| | | | GSLRL | | | | | | | | SLRAED | | | | |
| | | | SCAVS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81544 | 1286 | TGGGTCCTCGGTGAAGGTCTCCTGCAAGGCTTCTGGAGGCC | | | | | | 6996 | QVQLVQSGAEVKKP | | 7347 | 13 | | 9 | |
| | | CCTTCGGCAGTTCTCCAATCAGTTGGGTGCGGCAGGCCCCT | | | | | | | GSSVKVSCKASGGP | | | | | | |
| | | GGACAAGGGCTTGAGTGGATGGGAAGGATCATCCCTATTCT | | | | | | | FGSSPISWVRQAPG | | | | | | |
| | | TGGTGTGGCGGACTACGCACAGAAGTTTCAGGGCAGAGTCA | | | | | | | QGLEWMGRIIPILG | | | | | | |
| | | CCATTACCGCGGACAGATCCACGAGCACAACCTACATGGAG | | | | | | | VADYAQKFQGRVTI | | | | | | |
| | | CTGAGCAGCCTGAGATCTGAAGACACGGCCCGATATTATTG | | | | | | | TADRSTSTTYMELS | | | | | | |
| | | TGCGAGAGATGTGGGTTACTCTGGTTCGGGGAGCTCTTACT | | | | | | | SLRSEDTARYYCAR | | | | | | |
| | | ACTTTGACCCCTGGGGCCAGGGAACCCTGGTCACCGTCTCC | | | | | | | DVGYSGSGSSYYFD | | | | | | |
| | | TCA | | | | | | | PWGQGTLVTVSS | | | | | | |
| ADI-81691 | 1294 | TGGGGGGTCCCTGAGACTCTCCTGTGTAGCCTCTGGATTCA | | | | | | 6997 | EVQLVESGGGLVQP | | 7348 | 13 | | 13 | |
| | | CCCTCAGTAACGACGACATACACTGGGTCCGCCAAGCTCCA | | | | | | | GGSLRLSCVASGFT | | | | | | |
| | | GGAAAAGGTCTGGAGTGGGTCTCAGCTATCGGTTCTGCCGG | | | | | | | LSNDDIHWVRQAPG | | | | | | |
| | | TGACACATATTATGCAGACTCCGAGAAGGGCCGATTCACCA | | | | | | | KGLEWVSAIGSAGD | | | | | | |
| | | TCTCCAGAGAAAAGGCCAACAACTCCTTGTATCTTCAAATG | | | | | | | TYYADSEKGRFTIS | | | | | | |
| | | CACAGCCTGAGAGCCGGGGACACGGCTGTGTATTACTGTGT | | | | | | | REKANNSLYLQMHS | | | | | | |
| | | GAGAGGGACGATGACTACGCTTGGGGTCTTCCAATACTATT | | | | | | | LRAGDTAVYYCVRG | | | | | | |
| | | ACAAGGAAATTTGGGGCAGAGGGACCACGGTCACCGTCTCC | | | | | | | TMTTLGVFQYYYKE | | | | | | |
| | | TCA | | | | | | | IWGRGTTVTVSS | | | | | | |
| ADI-81571 | 1286 | TGGGTCTTCGGTGAAGGTCTCCTGCAAGGCCTCTGGAGGCT | | | | | | 6998 | QVQLQQSGAEVKKP | | 7349 | 12 | | 10 | |
| | | CCTTCAACAGCCATAGCATCAGTTGGGTGCGACAGGCCCCT | | | | | | | GSSVKVSCKASGGS | | | | | | |
| | | GGACAAGGGCTTGAGTGGATGGGAAGGATCATCCCTATCCT | | | | | | | FNSHSISWVRQAPG | | | | | | |
| | | TGGTGTAGCAGACTACGCACAGAAGTTCCAGGGCAGAGTCA | | | | | | | QGLEWMGRIIPILG | | | | | | |
| | | ACATTATCGCGGACAAGTCCACGGGCACAGCCTACATGGAG | | | | | | | VADYAQKFQGRVNI | | | | | | |
| | | CTGAGGAGCCTGAGATCTGAGGACACGGCCGTGTATTACTG | | | | | | | IADKSTGTAYMELR | | | | | | |
| | | CGCGAGAGATGTGGGTTATTCTGGTTCGGGGAGTGCTTACT | | | | | | | SLRSEDTAVYYCAR | | | | | | |
| | | ACTTTGACCTCTGGGGCCAGGGAACCCTGGTCACCGTCTCC | | | | | | | DVGYSGSGSAYYFD | | | | | | |
| | | TCA | | | | | | | LWGQGTLVTVSS | | | | | | |
| ADI-81850 | 1286 | GAGGTGCAGCTGGTGGAGTCTGGGGGAGGCGTGGTCCAGCC | | | | | | 6999 | EVQLVESGGGVVQP | | 7350 | 12 | | 10 | |
| | | TGGGAGGTCCCTGAGACTCTCCTGTGCAGCCTTTGGATTCA | | | | | | | GRSLRLSCAAFGFT | | | | | | |
| | | CCTTCAGTACTTATGCCATGCACTGGGTCCGCCAGGCTCCA | | | | | | | FSTYAMHWVRQAPG | | | | | | |
| | | GGCAAGGGGCTGGAGTGGGTGGCAGTTATATCAGATGATGG | | | | | | | KGLEWVAVISDDGR | | | | | | |
| | | CAGATATAAAGACTCCGCAGACTCCGTGAAGGGCCGATTCA | | | | | | | YKDSADSVKGRFTI | | | | | | |
| | | CCATCTCCAGAGACAATTCCCAGAACACGCTTTATCTGCAG | | | | | | | SRDNSQNTLYLQMS | | | | | | |
| | | ATGAGCAGCCTGAGACCTGAGGACACAGCTATCTATTACTG | | | | | | | SLRPEDTAIYYCTR | | | | | | |
| | | TACGAGAGAGGGCCCTATGACTACGGTGACCTACTTTGACT | | | | | | | EGPMTTVTYFDYWG | | | | | | |
| | | ACTGGGGCCAGGGAACCCTGGTCACCGTCTCCTCA | | | | | | | QGTLVTVSS | | | | | | |
| ADI-81628 | 6819 | GAGGTGCAGCTGCAGCAGTCGGGCGCGGGACTGTTGAAGCC | | | | | | 7000 | EVQLQQSGAGLLKP | | 7351 | 11 | | 8 | |
| | | TTCGGAGACCCTGTCCCTCACCTGCGGTGTCTTTGGTGGGT | | | | | | | SETLSLTCGVFGGS | | | | | | |
| | | CCTTCAGTGGGAACTACTGGAGCTGGATCCGCCAGTCCCCA | | | | | | | FSGNYWSWIRQSPG | | | | | | |
| | | GGGAAGGGGCTGGAGTGGATTGGCGAGATCAATCATAGCGG | | | | | | | KGLEWIGEINHSGT | | | | | | |
| | | AACCACCACTTACAACCCGTCCCTCAAGAGTCGAGTCACCA | | | | | | | TTYNPSLKSRVTIS | | | | | | |
| | | TATCAATTGACACGTCCAAGAACCAGTTCTCCCTGCGGCTG | | | | | | | IDTSKNQFSLRLSS | | | | | | |
| | | AGCTCTGTGACCGCCGCGGACACGGCTGTGTATTACTGTGC | | | | | | | VTAADTAVYYCARE | | | | | | |
| | | GAGAGAGGTTGTAGTAATACCAGCTGCTACAATAAGGTACT | | | | | | | VVVIPAATIRYSYN | | | | | | |
| | | CCTACAACTACTACATGGACGTCTGGGGCGCAGGGACCACG | | | | | | | YYMDVWGAGTTVTV | | | | | | |
| | | GTCACCGTCTCCTCA | | | | | | | SS | | | | | | |
| ADI-81678 | 6820 | GAGGTGCAGCTGTTGGAGTCCGGAGCAGAGGTGAAAAAGCC | | | | | | 7001 | EVQLLESGAEVKKP | | 7352 | 11 | | 10 | |
| | | CGGGGAGTCTCTGAGGATCTCCTGTAAGGGTTCTGGATACA | | | | | | | GESLRISCKGSGYS | | | | | | |
| | | GTTTCACCACCTACTGGATCACCTGGGTGCGCCAGATGCCC | | | | | | | FTTYWITWVRQMPG | | | | | | |
| | | GGGAAAGGCCTGGAATGGATGGGGAGGATTGATCCTAGTGA | | | | | | | KGLEWMGRIDPSDS | | | | | | |
| | | CTCTTACACCAAATACAGTCCGTCCTTCCAAGGCCGAGTAT | | | | | | | YTKYSPSFQGRVSI | | | | | | |
| | | CCATCTCGGCTGACAAGTCCACCAGCAATGCCTACATGCAG | | | | | | | SADKSTSNAYMQWS | | | | | | |
| | | TGGAGCAGCCTCCAGGCCTCGGACACCGCCATTTATTACTG | | | | | | | SLQASDTAIYYCAR | | | | | | |
| | | TGCGAGGGGGCGAGGGAGTGCCTACGACATAATGGAAATTG | | | | | | | GRGSAYDIMEIEYW | | | | | | |
| | | AGTATTGGGGCCAGGGAACCCTGGTCACGGCTCCTCAG | | | | | | | GQGTLVTAPQ | | | | | | |
| ADI-81738 | 1286 | CAGGTCCAGCTGGTGCAGTCTGGAGCAGAGGTGAGAAAGCC | | | | | | 7002 | QVQLVQSGAEVRKP | | 7353 | 11 | | 8 | |
| | | CGGGGAGTCTCTGAAGATCTCCTGTAAGGCTTCTGGATACA | | | | | | | GESLKISCKASGYT | | | | | | |
| | | CGTTTAGTACCTACTGGATCGGCTGGGTGCGCCAGATGCCC | | | | | | | FSTYWIGWVRQMPG | | | | | | |
| | | GGGAAAGGCCTGGAGTGGATGGGGATCATCTTTCCTGGTGA | | | | | | | KGLEWMGIIFPGDS | | | | | | |
| | | CTCTGATACCAGGTACAGCCCGTCCTTCCAAGGCCAGGTCA | | | | | | | DTRYSPSFQGQVTI | | | | | | |
| | | CCATCTCAGCCGACAAGTCCATCAGCACCGCCTACGTACAG | | | | | | | SADKSISTAYVQWS | | | | | | |
| | | TGGAGCAGCCTGAAGGCCTCGGACACCGCCATGTATTTCTG | | | | | | | SLKASDTAMYFCAT | | | | | | |
| | | TGCGACTCTGAAACTCGAGGGATTTACCCCCGTTGACTACA | | | | | | | LKLEGFTPVDYNTL | | | | | | |
| | | ATACACTTCCTGCTCTTTACTGGGGCCAGGGAACCCTGGTC | | | | | | | PALYWGQGTLVTVS | | | | | | |
| | | ACCGTCTCCTCA | | | | | | | S | | | | | | |
| ADI-81667 | 1299 | CAGGTGCAGCTGGTGGAGTCTGGGGGAGACTTGGTCCAGCC | | | | | | 7003 | QVQLVESGGDLVQP | | 7354 | 10 | | 9 | |
| | | TGGGGGGTCCCTGAGACTCTCCTGTGCAGTCTCTGGATTCA | | | | | | | GGSLRLSCAVSGFT | | | | | | |
| | | CCGTCAGTAACAACTACATGAGCTGGGTCCGCCAGCCTCCC | | | | | | | VSNNYMSWVRQPPG | | | | | | |
| | | GGGAAGGGGCTGGAGTGGGTCTCATTTATTTATAGCGGTGG | | | | | | | KGLEWVSFIYSGGN | | | | | | |
| | | TAACACATTCTACGCAGACTCCGTGAAGGGCCGATTCACCA | | | | | | | TFYADSVKGRFTIT | | | | | | |
| | | TCACCAGAGACAATTCCAAGAACACACTGTGGCTTCAAATG | | | | | | | RDNSKNTLWLQMNS | | | | | | |
| | | AACAGCCTGAGAGCCGAAGACACGGCTGTTTACTACTGTGC | | | | | | | LRAEDTAVYYCATS | | | | | | |
| | | GACCTCACCTGCGGTGGGGGTCTGGGGCCAAGGGACAATGG | | | | | | | PAVGVWGQGTMVTV | | | | | | |
| | | TCACCGTCTCCTCA | | | | | | | SS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81832 | 11 | IGHV3- | QVTLK | 106 | FTFNKY | 5648 | WVRQAP | 5830 | VISYDG | 6020 | RFTISR | 6334 | VKVQGEY | 6640 | WGQGTT |
| | | 30 | ESGGG | | GMH | | GRGLEW | | TNEFYA | | DNSKNK | | CSGTSCY | | VTVSS |
| | | | VVQPG | | | | LA | | DFVKG | | LYLQMN | | GFTLDV | | |
| | | | RSLRL | | | | | | | | SLRAED | | | | |
| | | | SCAAS | | | | | | | | TAVYYC | | | | |
| | | | G | | | | | | | | | | | | |
| ADI-81711 | 81 | IGHV3- | QVQLV | 11 | FTFRIY | 5649 | WVRQAP | 5831 | VISFDG | 6021 | RFTISR | 6335 | AKGSAVP | 6641 | WGQGTL |
| | | 30 | ESGGG | | GMH | | GKGLES | | SDRYYA | | DNSKNT | | GSRPYYR | | VTVSS |
| | | | VVQPG | | | | VA | | DSVKG | | LYLQMN | | DF | | |
| | | | RSLRL | | | | | | | | SLRAQD | | | | |
| | | | SCAAS | | | | | | | | TAVYYC | | | | |
| | | | G | | | | | | | | | | | | |
| ADI-81762 | 38 | IGHV4- | EVQLV | 5433 | GSISSS | 5650 | WVRQSP | 5832 | EIYQSG | 6022 | RVTISI | 6336 | ARTPLTD | 6642 | WGKGTT |
| | | 4 | ESGPG | | NWWS | | GKGLEW | | STNDNP | | DKSKNQ | | FYYMDV | | VTVSS |
| | | | LVKPS | | | | IA | | SLKS | | FSLKLS | | | | |
| | | | GTLSL | | | | | | | | YVTAAD | | | | |
| | | | TCTVS | | | | | | | | TAVYFC | | | | |
| | | | G | | | | | | | | | | | | |
| ADI-81626 | 82 | IGHV4- | QVQLQ | 21 | ASISST | 5651 | WVRQPP | 444 | EIYHSG | 6023 | RVTMSV | 6337 | ARGVGFD | 6643 | WGRGTL |
| | | 4 | ESGPG | | NWWS | | GKGLEW | | STNYNT | | DKSQNH | | DFWSGPN | | VTVSS |
| | | | LVKPS | | | | IG | | SLES | | FSLKLR | | RYFSL | | |
| | | | GTLSL | | | | | | | | SVTAAD | | | | |
| | | | TCAVS | | | | | | | | TAVYYC | | | | |
| | | | G | | | | | | | | | | | | |
| ADI-81803 | 79 | IGHV1- | EVQLL | 5434 | GAFSIY | 5652 | WVRQAP | 433 | EIIPIT | 6024 | RVTITA | 1006 | ARVGRSY | 6644 | WGQGTL |
| | | 69 | ESGAE | | AIS | | GQGLEW | | GTANYA | | DESTST | | YDSSGYY | | VTVSS |
| | | | VRKPG | | | | MG | | QKFQG | | AYMELS | | YTYFDS | | |
| | | | SSVRV | | | | | | | | SLRSED | | | | |
| | | | SCKAS | | | | | | | | TAVYYC | | | | |
| | | | G | | | | | | | | | | | | |
| ADI-81882 | 36 | IGHV3- | EVQLL | 5373 | FTFINY | 5626 | WVRQAP | 431 | AISYDG | 5997 | RFTISR | 6338 | AKVTASH | 6613 | WGQGTT |
| | | 30 | ESGGG | | GMH | | GKGLEW | | SDEYYA | | DDSKNT | | YYYYYGM | | VTVSS |
| | | | VVQPG | | | | VA | | DSVKG | | VYLQMN | | DV | | |
| | | | RSLRL | | | | | | | | SLRTED | | | | |
| | | | SCAAS | | | | | | | | TAVYYC | | | | |
| | | | G | | | | | | | | | | | | |
| ADI-81911 | 85 | IGHV3- | EVQLL | 5392 | FTFSNY | 233 | WVRQAP | 431 | FISHDG | 6025 | RFTISR | 6339 | AKHFGAP | 6645 | WGQGTT |
| | | 30 | ESGGG | | GMH | | GKGLEW | | STKYYA | | DTSKNT | | TSYYYYG | | VTVSS |
| | | | VVQPG | | | | VA | | DSVKD | | LYLQMN | | MDV | | |
| | | | RSLRL | | | | | | | | NLRAED | | | | |
| | | | SCAVS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81832 | 1287 | CAGGTGACGTTGAAGGAGTCTGGGGGAGGCGTGGTCCAGCC | | | | | | 7004 | QVTLKESGGGVVQP | | 7355 | 10 | | 9 | |
| | | TGGGAGGTCCCTGAGACTCTCCTGTGCAGCCTCTGGATTCA | | | | | | | GRSLRLSCAASGFT | | | | | | |
| | | CCTTCAATAAATATGGCATGCACTGGGTCCGCCAGGCTCCA | | | | | | | FNKYGMHWVRQAPG | | | | | | |
| | | GGCAGGGGGCTGGAGTGGCTGGCAGTTATATCATATGATGG | | | | | | | RGLEWLAVISYDGT | | | | | | |
| | | AACTAATGAATTCTATGCAGACTTCGTGAAGGGCCGATTCA | | | | | | | NEFYADFVKGRFTI | | | | | | |
| | | CCATCTCCAGAGACAATTCCAAGAACAAGTTGTATCTGCAA | | | | | | | SRDNSKNKLYLQMN | | | | | | |
| | | ATGAACAGCCTGAGAGCTGAGGACACGGCTGTCTATTACTG | | | | | | | SLRAEDTAVYYCVK | | | | | | |
| | | TGTGAAAGTCCAAGGGGAATATTGTAGTGGTACCAGCTGCT | | | | | | | VQGEYCSGTSCYGF | | | | | | |
| | | ACGGGTTCACTTTGGACGTCTGGGGCCAAGGGACCACGGTC | | | | | | | TLDVWGQGTTVTVS | | | | | | |
| | | ACCGTCTCCTCA | | | | | | | S | | | | | | |
| ADI-81711 | 1286 | CAGGTGCAGCTGGTGGAGTCTGGGGGAGGCGTGGTCCAGCC | | | | | | 7005 | QVQLVESGGGVVQP | | 7356 | 9 | | 7 | |
| | | TGGGAGGTCCCTGAGACTCTCCTGTGCAGCCTCTGGATTCA | | | | | | | GRSLRLSCAASGFT | | | | | | |
| | | CCTTCAGAATTTATGGCATGCACTGGGTCCGCCAGGCTCCA | | | | | | | FRIYGMHWVRQAPG | | | | | | |
| | | GGCAAGGGGCTGGAGTCCGTGGCAGTTATCTCATTTGATGG | | | | | | | KGLESVAVISFDGS | | | | | | |
| | | AAGTGATAGATATTACGCAGACTCCGTGAAGGGCCGATTCA | | | | | | | DRYYADSVKGRFTI | | | | | | |
| | | CCATCTCCAGAGACAATTCCAAGAACACGCTGTATCTGCAA | | | | | | | SRDNSKNTLYLQMN | | | | | | |
| | | ATGAACAGCCTGAGGGCTCAGGACACGGCTGTCTATTACTG | | | | | | | SLRAQDTAVYYCAK | | | | | | |
| | | TGCGAAAGGATCTGCAGTGCCTGGTTCACGGCCCTACTACT | | | | | | | GSAVPGSRPYYFDF | | | | | | |
| | | TTGACTTCTGGGGCCAGGGAACCCTGGTCACCGTCTCCTCA | | | | | | | WGQGTLVTVSS | | | | | | |
| ADI-81762 | 1291 | GAGGTGCAGCTGGTGGAGTCGGGCCCAGGACTGGTGAAGCC | | | | | | 7006 | EVQLVESGPGLVKP | | 7357 | 9 | | 8 | |
| | | TTCGGGGACCCTGTCCCTCACCTGCACTGTCTCTGGTGGCT | | | | | | | SGTLSLTCTVSGGS | | | | | | |
| | | CCATCAGCAGTAGCAATTGGTGGAGCTGGGTCCGCCAGTCT | | | | | | | ISSSNWWSWVRQSP | | | | | | |
| | | CCTGGGAAGGGGCTGGAGTGGATTGCGGAAATCTATCAGAG | | | | | | | GKGLEWIAEIYQSG | | | | | | |
| | | TGGGAGCACCAACGACAACCCGTCCCTCAAGAGTCGAGTCA | | | | | | | STNDNPSLKSRVTI | | | | | | |
| | | CCATATCAATAGACAAGTCCAAGAACCAGTTCTCCCTGAAG | | | | | | | SIDKSKNQFSLKLS | | | | | | |
| | | TTGAGTTATGTGACCGCCGCGGACACGGCCGTGTATTTCTG | | | | | | | YVTAADTAVYFCAR | | | | | | |
| | | TGCGAGGACCCCCCTTACGGACTTCTACTACATGGACGTCT | | | | | | | TPLTDFYYMDVWGK | | | | | | |
| | | GGGGCAAAGGGACCACGGTCACCGTCTCCTCA | | | | | | | GTTVTVSS | | | | | | |
| ADI-81626 | 1288 | TTCGGGGACCCTGTCCCTCACCTGCGCTGTCTCTGGTGCCT | | | | | | 7007 | QVQLQESGPGLVKP | | 7358 | 8 | | 8 | |
| | | CCATCAGCAGTACTAACTGGTGGAGTTGGGTCCGCCAGCCC | | | | | | | SGTLSLTCAVSGAS | | | | | | |
| | | CCAGGGAAGGGGCTGGAGTGGATTGGAGAAATCTATCATAG | | | | | | | ISSTNWWSWVRQPP | | | | | | |
| | | TGGGAGCACCAACTACAACACGTCCCTCGAGAGTCGAGTCA | | | | | | | GKGLEWIGEIYHSG | | | | | | |
| | | CCATGTCAGTGGACAAATCCCAGAACCACTTCTCCCTGAAA | | | | | | | STNYNTSLESRVTM | | | | | | |
| | | CTGAGGTCTGTGACCGCCGCGGACACGGCCGTGTATTACTG | | | | | | | SVDKSQNHFSLKLR | | | | | | |
| | | TGCGAGAGGTGTGGGATTTGACGATTTTTGGAGTGGTCCCA | | | | | | | SVTAADTAVYYCAR | | | | | | |
| | | ACAGGTACTTCAGTCTCTGGGGCCGTGGCACCCTGGTCACC | | | | | | | GVGFDDFWSGPNRY | | | | | | |
| | | GTCTCCTCA | | | | | | | FSLWGRGTLVTVSS | | | | | | |
| ADI-81803 | 1286 | GAGGTGCAGCTGTTGGAGTCTGGGGCTGAGGTGAGGAAGCC | | | | | | 7008 | EVQLLESGAEVRKP | | 7359 | 8 | | 6 | |
| | | TGGGTCCTCGGTGAGGGTCTCCTGCAAGGCTTCTGGAGGCG | | | | | | | GSSVRVSCKASGGA | | | | | | |
| | | CCTTCAGCATTTATGCCATCAGCTGGGTGCGACAGGCCCCT | | | | | | | FSIYAISWVRQAPG | | | | | | |
| | | GGTCAAGGGCTTGAGTGGATGGGAGAGATCATCCCTATCAC | | | | | | | QGLEWMGEIIPITG | | | | | | |
| | | TGGTACAGCAAACTACGCACAGAAGTTCCAGGGCAGAGTCA | | | | | | | TANYAQKFQGRVTI | | | | | | |
| | | CGATTACCGCGGACGAATCCACGAGCACAGCCTACATGGAA | | | | | | | TADESTSTAYMELS | | | | | | |
| | | CTGAGCAGCCTGAGATCTGAGGACACGGCCGTGTATTACTG | | | | | | | SLRSEDTAVYYCAR | | | | | | |
| | | TGCGAGAGTCGGTCGCTCTTACTATGATAGTAGTGGTTATT | | | | | | | VGRSYYDSSGYYYT | | | | | | |
| | | ACTACACATACTTTGACTCCTGGGGCCAGGGAACCCTGGTC | | | | | | | YFDSWGQGTLVTVS | | | | | | |
| | | ACCGTCTCCTCA | | | | | | | S | | | | | | |
| ADI-81882 | 1287 | GAGGTGCAGCTGTTGGAGTCTGGGGGAGGCGTGGTCCAGCC | | | | | | 7009 | EVQLLESGGGVVQP | | 7360 | 8 | | 8 | |
| | | TGGGAGGTCCCTGAGACTCTCCTGTGCAGCCTCTGGATTCA | | | | | | | GRSLRLSCAASGFT | | | | | | |
| | | CCTTCATTAACTATGGCATGCACTGGGTCCGCCAGGCTCCA | | | | | | | FINYGMHWVRQAPG | | | | | | |
| | | GGCAAGGGGCTGGAGTGGGTGGCAGCTATATCATATGATGG | | | | | | | KGLEWVAAISYDGS | | | | | | |
| | | AAGTGATGAATACTATGCAGACTCCGTGAAGGGCCGATTCA | | | | | | | DEYYADSVKGRFTI | | | | | | |
| | | CCATCTCCAGAGACGATTCCAAGAACACGGTGTATCTGCAA | | | | | | | SRDDSKNTVYLQMN | | | | | | |
| | | ATGAACAGCCTGAGAACTGAGGACACGGCTGTGTATTACTG | | | | | | | SLRTEDTAVYYCAK | | | | | | |
| | | TGCGAAAGTGACCGCCTCCCATTATTACTACTACTACGGGA | | | | | | | VTASHYYYYYGMDV | | | | | | |
| | | TGGACGTCTGGGGCCAAGGGACCACGGTCACCGTCTCCTCA | | | | | | | WGQGTTVTVSS | | | | | | |
| ADI-81911 | 1287 | TGGGAGGTCCCTGAGACTCTCCTGTGCAGTCTCTGGATTCA | | | | | | 7010 | EVQLLESGGGVVQP | | 7361 | 8 | | 8 | |
| | | CCTTCAGTAACTATGGCATGCACTGGGTCCGCCAGGCTCCA | | | | | | | GRSLRLSCAVSGFT | | | | | | |
| | | GGCAAGGGGCTGGAGTGGGTGGCATTTATATCACATGATGG | | | | | | | FSNYGMHWVRQAPG | | | | | | |
| | | AAGTACTAAATACTATGCAGACTCCGTGAAGGACCGATTCA | | | | | | | KGLEWVAFISHDGS | | | | | | |
| | | CCATCTCCAGAGACACTTCCAAGAACACACTGTATCTGCAA | | | | | | | TKYYADSVKDRFTI | | | | | | |
| | | ATGAACAACCTGAGAGCTGAGGACACGGCTGTGTATTACTG | | | | | | | SRDTSKNTLYLQMN | | | | | | |
| | | CGCGAAGCATTTCGGGGCCCCTACTTCCTACTACTACTACG | | | | | | | NLRAEDTAVYYCAK | | | | | | |
| | | GTATGGACGTCTGGGGCCAAGGGACCACGGTCACCGTCTCC | | | | | | | HFGAPTSYYYYGMD | | | | | | |
| | | TCA | | | | | | | VWGQGTTVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81557 | 23 | IGHV3- | QVQLV | 10 | FTFSTY | 215 | WVRQAP | 5833 | LISYDG | 6026 | RFTISR | 6340 | AKALGPY | 6646 | WGQGTL |
| | | 30 | QSGGG | | GMH | | GKGLEW | | SNTYSA | | DNPKNT | | CGAGDCY | | VTVSS |
| | | | VVQPG | | | | LA | | DSVKG | | LFLQMN | | WAPLDF | | |
| | | | RSLRL | | | | | | | | SLRAED | | | | |
| | | | SCAAS | | | | | | | | TAVYYC | | | | |
| | | | G | | | | | | | | | | | | |
| ADI-81886 | 62 | IGHV3- | QVQLV | 5435 | FTFDDY | 214 | WVRLVP | 5834 | GISWNS | 6027 | RFIISR | 6341 | GKDKGPY | 6647 | WGQGTT |
| | | 9 | QSGGG | | AMH | | GKGLEW | | GTIGYA | | DNAENS | | YYDSGSL | | VTVSS |
| | | | LVQPG | | | | VS | | DSVKG | | LYLQMN | | WDYYSNG | | |
| | | | RSLRL | | | | | | | | SLRAED | | MDV | | |
| | | | SCAGS | | | | | | | | SALYYC | | | | |
| | | | G | | | | | | | | | | | | |
| ADI-81819 | 93 | IGHV4- | EVQLV | 5436 | VSISSG | 5653 | WIRQHP | 450 | YIYYSG | 611 | RVTMSV | 6342 | ARDYGGN | 6648 | WGQGTL |
| | | 31 | ESGPG | | GYYWN | | GKGLEW | | STYYNP | | DTSKNQ | | AQYFNY | | VTVSS |
| | | | LVKPS | | | | IG | | SLKS | | YSLKLS | | | | |
| | | | QTLSL | | | | | | | | SLTAAD | | | | |
| | | | TCTVS | | | | | | | | TAVYYC | | | | |
| | | | G | | | | | | | | | | | | |
| ADI-81621 | 78 | IGHV3- | QVQLE | 109 | FTFSSY | 5654 | WVRQAP | 437 | TISGSG | 6028 | RFTISR | 6343 | AKANDLG | 6649 | WGQGTT |
| | | 23 | QSGGG | | AMN | | GKGLEW | | GSTYYA | | DNSKNT | | YCSGGRC | | VTVSS |
| | | | LVQPG | | | | VS | | DSVKG | | LDLHMS | | YPPYYYY | | |
| | | | GSLRL | | | | | | | | SLRAED | | GMDV | | |
| | | | SCAAS | | | | | | | | TAVYYC | | | | |
| | | | G | | | | | | | | | | | | |
| ADI-81821 | 2 | IGHV1- | QVQLV | 50 | GTFSSY | 379 | WVRQAP | 433 | RIIPIL | 5999 | RVTIIA | 936 | ARDQGYS | 6650 | WGQGTL |
| | | 69 | QSGAE | | PIS | | GQGLEW | | GIPNYA | | DKSTST | | GSGSHYW | | VTVSS |
| | | | VKKPG | | | | MG | | QKFQG | | AYMELS | | FDP | | |
| | | | SSVKV | | | | | | | | SLRSED | | | | |
| | | | SCKAS | | | | | | | | TAVYYC | | | | |
| | | | G | | | | | | | | | | | | |
| ADI-81857 | 35 | IGHV4- | EVQLL | 5380 | ASISSY | 5655 | WIRQPP | 445 | YVYYSG | 6029 | RVTISV | 6344 | ATDYFDS | 6651 | WGQGTT |
| | | 59 | ESGPG | | YWS | | GKGLEW | | STNYNP | | DTSKNQ | | TGFHYGM | | VTVSS |
| | | | LVKPS | | | | IG | | SLKS | | FSLKLR | | DV | | |
| | | | ETLSL | | | | | | | | SVTAAD | | | | |
| | | | TCSVS | | | | | | | | TAVYYC | | | | |
| | | | G | | | | | | | | | | | | |
| ADI-81909 | 83 | IGHV5- | EVQLL | 5437 | YSFSSY | 5656 | WVRQMP | 476 | RIDPSD | 766 | HVTISA | 6345 | ARSGGNR | 6652 | WGQGTT |
| | | 10-1 | ESGAE | | WIS | | GKGLEW | | SYTNYS | | DKSIST | | LLYGMDV | | VTVSS |
| | | | VKKPG | | | | MG | | PSFQG | | AYLQWS | | | | |
| | | | ESLRI | | | | | | | | SLKASD | | | | |
| | | | SCKGS | | | | | | | | TAMYYC | | | | |
| | | | E | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81557 | 1286 | CAGGTGCAGCTGGTGCAGTCTGGGGGAGGCGTGGTCCAGCC | | | | | | 7011 | QVQLVQSGGGVVQP | | 7362 | 7 | | 7 | |
| | | TGGGAGGTCCCTAAGACTCTCCTGTGCAGCCTCTGGATTCA | | | | | | | GRSLRLSCAASGFT | | | | | | |
| | | CCTTCAGTACCTACGGCATGCACTGGGTCCGCCAGGCTCCA | | | | | | | FSTYGMHWVRQAPG | | | | | | |
| | | GGCAAGGGGCTGGAGTGGCTGGCACTTATTTCATATGATGG | | | | | | | KGLEWLALISYDGS | | | | | | |
| | | AAGTAATACATACTCTGCAGACTCCGTGAAGGGCCGATTCA | | | | | | | NTYSADSVKGRFTI | | | | | | |
| | | CCATCTCCAGAGACAATCCCAAGAACACTCTGTTTCTGCAA | | | | | | | SRDNPKNTLFLQMN | | | | | | |
| | | ATGAACAGCCTGAGAGCTGAGGACACGGCTGTGTATTACTG | | | | | | | SLRAEDTAVYYCAK | | | | | | |
| | | TGCGAAAGCCCTGGGCCCCTATTGTGGTGCTGGTGACTGCT | | | | | | | ALGPYCGAGDCYWA | | | | | | |
| | | ACTGGGCTCCCCTTGACTTCTGGGGCCAGGGAACCCTGGTC | | | | | | | PLDFWGQGTLVTVS | | | | | | |
| | | ACCGTCTCCTCA | | | | | | | S | | | | | | |
| ADI-81886 | 1287 | CAGGTGCAGCTGGTGCAGTCTGGGGGAGGCTTGGTACAGCC | | | | | | 7012 | QVQLVQSGGGLVQP | | 7363 | 7 | | 7 | |
| | | TGGCAGGTCCCTGAGACTCTCCTGTGCAGGCTCTGGATTCA | | | | | | | GRSLRLSCAGSGFT | | | | | | |
| | | CCTTTGATGATTATGCCATGCACTGGGTCCGGCTAGTTCCA | | | | | | | FDDYAMHWVRLVPG | | | | | | |
| | | GGGAAGGGCCTGGAGTGGGTCTCAGGTATTAGTTGGAATAG | | | | | | | KGLEWVSGISWNSG | | | | | | |
| | | TGGAACCATAGGCTATGCGGACTCTGTGAAGGGCCGATTCA | | | | | | | TIGYADSVKGRFII | | | | | | |
| | | TCATCTCCAGAGACAACGCCGAGAACTCCCTGTATTTGCAA | | | | | | | SRDNAENSLYLQMN | | | | | | |
| | | ATGAACAGTCTGAGAGCTGAGGACTCGGCCCTGTATTACTG | | | | | | | SLRAEDSALYYCGK | | | | | | |
| | | TGGAAAAGATAAGGGACCGTATTACTATGATTCCGGGAGTC | | | | | | | DKGPYYYDSGSLWD | | | | | | |
| | | TTTGGGACTACTACTCCAACGGTATGGATGTCTGGGGCCAA | | | | | | | YYSNGMDVWGQGTT | | | | | | |
| | | GGGACCACGGTCACCGTCTCCTCA | | | | | | | VTVSS | | | | | | |
| ADI-81819 | 1286 | GAGGTGCAGCTGGTGGAGTCGGGCCCAGGACTGGTGAAGCC | | | | | | 7013 | EVQLVESGPGLVKP | | 7364 | 6 | | 6 | |
| | | TTCACAGACCCTGTCCCTCACCTGCACTGTGTCTGGTGTCT | | | | | | | SQTLSLTCTVSGVS | | | | | | |
| | | CCATCAGCAGTGGTGGTTACTACTGGAACTGGATCCGCCAG | | | | | | | ISSGGYYWNWIRQH | | | | | | |
| | | CACCCAGGGAAGGGCCTGGAGTGGATTGGGTACATCTATTA | | | | | | | PGKGLEWIGYIYYS | | | | | | |
| | | CAGTGGGAGCACCTACTACAACCCGTCCCTCAAGAGTCGAG | | | | | | | GSTYYNPSLKSRVT | | | | | | |
| | | TTACCATGTCAGTAGACACGTCTAAGAACCAGTACTCCCTG | | | | | | | MSVDTSKNQYSLKL | | | | | | |
| | | AAGCTGAGCTCTCTGACTGCCGCGGACACGGCCGTGTATTA | | | | | | | SSLTAADTAVYYCA | | | | | | |
| | | CTGTGCGAGAGACTACGGTGGTAACGCCCAATACTTTAACT | | | | | | | RDYGGNAQYFNYWG | | | | | | |
| | | ACTGGGGCCAGGGAACCCTGGTCACCGTCTCCTCA | | | | | | | QGTLVTVSS | | | | | | |
| ADI-81621 | 1287 | CAGGTGCAGCTGGAGCAGTCTGGGGGAGGCTTGGTACAGCC | | | | | | 7014 | QVQLEQSGGGLVQP | | 7365 | 5 | | 5 | |
| | | GGGGGGGTCCCTGAGACTCTCCTGTGCAGCCTCTGGATTCA | | | | | | | GGSLRLSCAASGFT | | | | | | |
| | | CCTTCAGTAGCTATGCCATGAACTGGGTCCGCCAGGCTCCA | | | | | | | FSSYAMNWVRQAPG | | | | | | |
| | | GGGAAGGGACTGGAGTGGGTCTCAACTATTAGTGGTAGTGG | | | | | | | KGLEWVSTISGSGG | | | | | | |
| | | TGGTAGCACATACTACGCAGACTCCGTGAAGGGCCGGTTCA | | | | | | | STYYADSVKGRFTI | | | | | | |
| | | CCATCTCCAGAGACAATTCCAAGAACACGCTGGATCTACAC | | | | | | | SRDNSKNTLDLHMS | | | | | | |
| | | ATGAGCAGCCTGAGAGCCGAGGACACGGCCGTATATTACTG | | | | | | | SLRAEDTAVYYCAK | | | | | | |
| | | TGCGAAAGCTAATGACCTAGGATATTGTAGTGGTGGTAGGT | | | | | | | ANDLGYCSGGRCYP | | | | | | |
| | | GCTACCCCCCCTACTACTACTACGGTATGGACGTCTGGGGC | | | | | | | PYYYYGMDVWGQGT | | | | | | |
| | | CAAGGGACCACGGTCACCGTCTCCTCA | | | | | | | TVTVSS | | | | | | |
| ADI-81821 | 1286 | TGGGTCCTCGGTGAAGGTCTCCTGCAAGGCTTCTGGAGGCA | | | | | | 7015 | QVQLVQSGAEVKKP | | 7366 | 4 | | 3 | |
| | | CCTTCAGTAGCTATCCTATCAGCTGGGTGCGCCAGGCCCCT | | | | | | | GSSVKVSCKASGGT | | | | | | |
| | | GGACAAGGACTTGAGTGGATGGGAAGGATCATCCCTATCCT | | | | | | | FSSYPISWVRQAPG | | | | | | |
| | | TGGTATACCAAACTACGCACAGAAGTTCCAGGGCAGAGTCA | | | | | | | QGLEWMGRIIPILG | | | | | | |
| | | CGATTATTGCGGACAAATCCACGAGCACAGCCTACATGGAG | | | | | | | IPNYAQKFQGRVTI | | | | | | |
| | | TTGAGCAGCCTGAGATCTGAGGACACGGCCGTGTATTATTG | | | | | | | IADKSTSTAYMELS | | | | | | |
| | | TGCGAGAGATCAGGGTTACTCTGGTTCGGGGAGTCATTACT | | | | | | | SLRSEDTAVYYCAR | | | | | | |
| | | GGTTCGACCCCTGGGGCCAGGGAACCCTGGTCACCGTCTCC | | | | | | | DQGYSGSGSHYWFD | | | | | | |
| | | TCA | | | | | | | PWGQGTLVTVSS | | | | | | |
| ADI-81857 | 1287 | GAGGTGCAGCTGTTGGAGTCGGGCCCAGGACTGGTGAAGCC | | | | | | 7016 | EVQLLESGPGLVKP | | 7367 | 4 | | 4 | |
| | | TTCGGAGACCCTGTCCCTCACCTGCTCTGTCTCTGGTGCCT | | | | | | | SETLSLTCSVSGAS | | | | | | |
| | | CCATCAGTAGTTACTACTGGAGCTGGATCCGGCAGCCCCCA | | | | | | | ISSYYWSWIRQPPG | | | | | | |
| | | GGGAAGGGACTGGAGTGGATTGGGTATGTCTATTACAGTGG | | | | | | | KGLEWIGYVYYSGS | | | | | | |
| | | GAGCACCAACTACAACCCCTCCCTCAAGAGTCGAGTCACCA | | | | | | | TNYNPSLKSRVTIS | | | | | | |
| | | TATCAGTTGACACGTCCAAGAACCAGTTCTCCCTGAAGCTG | | | | | | | VDTSKNQFSLKLRS | | | | | | |
| | | AGGTCTGTGACCGCTGCGGACACGGCCGTTTATTACTGTGC | | | | | | | VTAADTAVYYCATD | | | | | | |
| | | GACTGATTACTTTGATAGTACTGGTTTCCACTACGGTATGG | | | | | | | YFDSTGFHYGMDVW | | | | | | |
| | | ACGTCTGGGGCCAAGGGACCACGGTCACCGTCTCCTCA | | | | | | | GQGTTVTVSS | | | | | | |
| ADI-81909 | 1287 | GAGGTGCAGCTGTTGGAGTCCGGAGCAGAGGTGAAAAAGCC | | | | | | 7017 | EVQLLESGAEVKKP | | 7368 | 2 | | 2 | |
| | | CGGGGAGTCTCTGAGGATCTCCTGTAAGGGTTCTGAATACA | | | | | | | GESLRISCKGSEYS | | | | | | |
| | | GCTTTTCCAGCTACTGGATCAGCTGGGTGCGCCAGATGCCC | | | | | | | FSSYWISWVRQMPG | | | | | | |
| | | GGGAAAGGCCTGGAGTGGATGGGGAGGATTGATCCTAGTGA | | | | | | | KGLEWMGRIDPSDS | | | | | | |
| | | CTCTTATACCAACTACAGCCCGTCCTTCCAAGGCCACGTCA | | | | | | | YTNYSPSFQGHVTI | | | | | | |
| | | CCATCTCAGCTGACAAGTCCATCAGCACTGCCTACCTGCAG | | | | | | | SADKSISTAYLQWS | | | | | | |
| | | TGGAGCAGCCTGAAGGCCTCGGACACCGCCATGTATTACTG | | | | | | | SLKASDTAMYYCAR | | | | | | |
| | | TGCGAGATCAGGTGGTAACCGGCTCCTTTATGGTATGGACG | | | | | | | SGGNRLLYGMDVWG | | | | | | |
| | | TCTGGGGCCAAGGGACCACGGTCACCGTCTCCTCA | | | | | | | QGTTVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81564 | 29 | IGHV1- | QVQLV | 5438 | SSFTHH | 5657 | WLRQAP | 5835 | RIIPIL | 6030 | RLTITA | 6346 | ARVGDMS | 6653 | WGQGTT |
| | | 69 | QSGAE | | PIA | | GQGLEW | | GVASDA | | DKSTTT | | TTGTDAF | | VTVSS |
| | | | VKKPG | | | | VG | | EKFQG | | TYMELN | | DI | | |
| | | | SSVKV | | | | | | | | SLRSED | | | | |
| | | | SCKPS | | | | | | | | TAVYFC | | | | |
| | | | G | | | | | | | | | | | | |
| ADI-81606 | 64 | IGHV3- | QVQLQ | 5439 | FTFSNY | 5658 | WVRQST | 5836 | GIGTSG | 6031 | RFTISR | 6347 | LFYYMDV | 6654 | WGKGTT |
| | | 13 | QSGGG | | DIH | | GEGLEW | | DTYYAE | | EDAMTS | | ARGDASG | | VTVSS |
| | | | LVRPG | | | | VS | | SVKG | | VYLQMN | | | | |
| | | | GSRRL | | | | | | | | RLTAGD | | | | |
| | | | SCAAS | | | | | | | | TAVYYC | | | | |
| | | | G | | | | | | | | | | | | |
| ADI-81782 | 58 | IGHV4- | EVQLL | 5365 | ASLSGI | 5659 | WVRQSP | 438 | EIFHTG | 6032 | RVTISI | 6348 | ARVAGLF | 6655 | WGQGSL |
| | | 4 | ESGPG | | NWWS | | GKGLEW | | SVQYNP | | DKSNSR | | DY | | VTVSS |
| | | | LVKPS | | | | IG | | SLKG | | FSLKLS | | | | |
| | | | GTLSL | | | | | | | | SVTAAD | | | | |
| | | | TCAVS | | | | | | | | TAVYYC | | | | |
| | | | G | | | | | | | | | | | | |
| ADI-81627 | 83 | IGHV3- | QVQLV | 57 | FSINTY | 5660 | WVRQAP | 431 | ILWFDG | 6033 | RFIISR | 6349 | ARDGEGG | 6656 | WGQGAL |
| | | 33 | QSGGG | | GIH | | GKGLEW | | VKKYYA | | DNFKNT | | AAEN | | VTVSS |
| | | | VVQPG | | | | VA | | ESVEG | | LYLQMN | | | | |
| | | | GSLRL | | | | | | | | SLRAED | | | | |
| | | | SCAAS | | | | | | | | TAVYFC | | | | |
| | | | G | | | | | | | | | | | | |
| ADI-81609 | 66 | IGHV1- | QVQLV | 5440 | GILNTY | 5661 | WVRQAP | 433 | RFIPII | 6034 | RVTITA | 6350 | ARAADPG | 6657 | WGQGTL |
| | | 69 | QSGAE | | SIS | | GQGLEW | | RTATYA | | DESTST | | VGPTIGY | | VTVSS |
| | | | MKKPG | | | | MG | | HKFQG | | AYMELN | | FDP | | |
| | | | SSVKV | | | | | | | | SLTYDD | | | | |
| | | | SCKAS | | | | | | | | TAVYYC | | | | |
| | | | G | | | | | | | | | | | | |
| ADI-81777 | 53 | IGHV1- | QVQLV | 50 | GTFSNY | 5662 | WVRQVP | 511 | RITPMF | 6035 | RVTFTA | 6351 | ARSEVMN | 6658 | WGQGTL |
| | | 69 | QSGAE | | GIN | | GQGLEW | | GMANYV | | DKSTGT | | VGHYYLD | | VTVSS |
| | | | VKKPG | | | | MG | | QKFQD | | AYLEVS | | S | | |
| | | | SSVKV | | | | | | | | SLTSED | | | | |
| | | | SCKAS | | | | | | | | TAVYYC | | | | |
| | | | G | | | | | | | | | | | | |
| ADI-81728 | 5 | IGHV4- | EVQLV | 5441 | GSIKSG | 5663 | WIRQHP | 450 | YIYFTG | 6036 | RVTISV | 6352 | ARVGGGG | 6659 | WGQGTL |
| | | 31 | ETGPG | | GYYWS | | GKGLEW | | STDYNP | | DTFENQ | | TNDFDF | | VTVSS |
| | | | LVKPS | | | | IG | | SLRG | | FSLRLT | | | | |
| | | | QTLSL | | | | | | | | SVTAAD | | | | |
| | | | TCSVS | | | | | | | | TATYYC | | | | |
| | | | G | | | | | | | | | | | | |
| ADI-81732 | 9 | IGHV3- | EVQLV | 5442 | FTFRTY | 5664 | WVRQTA | 5837 | TIGTSG | 6037 | RFTISR | 6353 | ARGNDIA | 6660 | WGKGTT |
| | | 13 | ESGGD | | DMH | | VKGLEW | | DRYYSD | | EDAKDS | | LVPPAMS | | VTVSS |
| | | | LVQPG | | | | VS | | SVKG | | LYLEMN | | ERPRNYF | | |
| | | | GSLRL | | | | | | | | SLRGGD | | YYLDV | | |
| | | | SCAAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81564 | 1287 | CAGGTGCAGCTGGTGCAGTCTGGGGCTGAGGTGAAGAAGCC | | | | | | 7018 | QVQLVQSGAEVKKP | | 7369 | 21 | | 19 | |
| | | TGGGTCCTCGGTGAAGGTCTCCTGCAAGCCTTCTGGAAGCT | | | | | | | GSSVKVSCKPSGSS | | | | | | |
| | | CCTTCACCCACCATCCTATCGCCTGGCTGCGACAGGCCCCT | | | | | | | FTHHPIAWLRQAPG | | | | | | |
| | | GGACAAGGGCTTGAGTGGGTGGGAAGGATCATCCCTATCCT | | | | | | | QGLEWVGRIIPILG | | | | | | |
| | | TGGTGTAGCCAGCGACGCAGAGAAGTTCCAGGGCCGACTCA | | | | | | | VASDAEKFQGRLTI | | | | | | |
| | | CGATTACCGCGGACAAATCCACGACCACTACCTACATGGAG | | | | | | | TADKSTTTTYMELN | | | | | | |
| | | CTGAACAGCCTGAGATCTGAGGACACGGCCGTGTATTTCTG | | | | | | | SLRSEDTAVYFCAR | | | | | | |
| | | TGCGAGAGTGGGGGACATGTCTACAACCGGGACTGATGCTT | | | | | | | VGDMSTTGTDAFDI | | | | | | |
| | | TTGATATCTGGGGCCAAGGGACCACGGTCACCGTCTCCTCA | | | | | | | WGQGTTVTVSS | | | | | | |
| ADI-81606 | 1291 | CAGGTGCAGCTGCAGCAGTCTGGGGGAGGCTTGGTACGACC | | | | | | 7019 | QVQLQQSGGGLVRP | | 7370 | 20 | | 16 | |
| | | GGGGGGGTCCCGGAGACTCTCCTGTGCAGCCTCTGGATTCA | | | | | | | GGSRRLSCAASGFT | | | | | | |
| | | CCTTCAGTAACTATGACATACACTGGGTCCGCCAAAGTACA | | | | | | | FSNYDIHWVRQSTG | | | | | | |
| | | GGAGAAGGTCTGGAATGGGTCTCAGGTATTGGTACTTCTGG | | | | | | | EGLEWVSGIGTSGD | | | | | | |
| | | TGACACATACTATGCAGAGTCCGTGAAGGGCCGCTTCACCA | | | | | | | TYYAESVKGRFTIS | | | | | | |
| | | TCTCCAGAGAAGACGCCATGACCTCCGTGTATCTTCAAATG | | | | | | | REDAMTSVYLQMNR | | | | | | |
| | | AACAGGCTGACAGCCGGGGACACGGCTGTGTATTACTGTGC | | | | | | | LTAGDTAVYYCARG | | | | | | |
| | | AAGGGGAGACGCATCAGGCCTCTTCTACTACATGGACGTCT | | | | | | | DASGLFYYMDVWGK | | | | | | |
| | | GGGGCAAAGGGACCACGGTCACCGTCTCCTCA | | | | | | | GTTVTVSS | | | | | | |
| ADI-81782 | 1289 | GAGGTGCAGCTGTTGGAGTCGGGCCCAGGACTGGTGAAGCC | | | | | | 7020 | EVQLLESGPGLVKP | | 7371 | 20 | | 14 | |
| | | TTCGGGGACCCTGTCCCTCACCTGCGCTGTCTCTGGTGCTT | | | | | | | SGTLSLTCAVSGAS | | | | | | |
| | | CCCTCAGCGGTATTAATTGGTGGAGTTGGGTCCGCCAGTCC | | | | | | | LSGINWWSWVRQSP | | | | | | |
| | | CCAGGGAAGGGCCTGGAGTGGATTGGAGAAATCTTTCATAC | | | | | | | GKGLEWIGEIFHTG | | | | | | |
| | | TGGGAGTGTCCAGTACAACCCGTCCCTCAAGGGTCGAGTCA | | | | | | | SVQYNPSLKGRVTI | | | | | | |
| | | CCATTTCAATCGACAAGTCGAACAGTCGTTTCTCCCTGAAG | | | | | | | SIDKSNSRFSLKLS | | | | | | |
| | | CTGAGCTCTGTGACCGCCGCGGACACGGCCGTTTATTACTG | | | | | | | SVTAADTAVYYCAR | | | | | | |
| | | TGCGAGAGTGGCTGGACTTTTTGACTACTGGGGCCAGGGAT | | | | | | | VAGLFDYWGQGSLV | | | | | | |
| | | CCCTGGTCACCGTCTCCTCA | | | | | | | TVSS | | | | | | |
| ADI-81627 | 1285 | CAGGTGCAGCTGGTGCAGTCTGGGGGAGGCGTGGTCCAGCC | | | | | | 7021 | QVQLVQSGGGVVQP | | 7372 | 19 | | 16 | |
| | | TGGGGGGTCCCTGAGACTCTCCTGTGCAGCGTCTGGTTTCA | | | | | | | GGSLRLSCAASGFS | | | | | | |
| | | GCATCAATACCTATGGCATACACTGGGTCCGCCAGGCTCCA | | | | | | | INTYGIHWVRQAPG | | | | | | |
| | | GGCAAGGGACTGGAGTGGGTGGCAATTTTGTGGTTTGATGG | | | | | | | KGLEWVAILWFDGV | | | | | | |
| | | AGTTAAGAAATACTATGCAGAGTCCGTGGAGGGCCGCTTCA | | | | | | | KKYYAESVEGRFII | | | | | | |
| | | TCATTTCCAGAGACAATTTCAAGAACACGCTGTATCTACAA | | | | | | | SRDNFKNTLYLQMN | | | | | | |
| | | ATGAACAGCCTGAGAGCCGAGGACACGGCTGTGTATTTTTG | | | | | | | SLRAEDTAVYFCAR | | | | | | |
| | | TGCGAGAGATGGAGAGGGTGGGGCCGCTGAAAACTGGGGCC | | | | | | | DGEGGAAENWGQGA | | | | | | |
| | | AGGGAGCCCTGGTCACCGTCTCCTCA | | | | | | | LVTVSS | | | | | | |
| ADI-81609 | 1286 | TGGGTCCTCGGTGAAGGTCTCCTGCAAGGCTTCTGGAGGCA | | | | | | 7022 | QVQLVQSGAEMKKP | | 7373 | 18 | | 16 | |
| | | TCCTCAACACCTATAGTATTTCCTGGGTGCGACAGGCCCCT | | | | | | | GSSVKVSCKASGGI | | | | | | |
| | | GGACAAGGGCTTGAGTGGATGGGAAGGTTCATCCCTATCAT | | | | | | | LNTYSISWVRQAPG | | | | | | |
| | | TCGAACAGCAACCTACGCACATAAGTTCCAGGGCAGAGTCA | | | | | | | QGLEWMGRFIPIIR | | | | | | |
| | | CGATTACCGCGGACGAGTCCACGAGCACAGCCTACATGGAG | | | | | | | TATYAHKFQGRVTI | | | | | | |
| | | CTGAACAGCCTGACATATGACGACACGGCCGTCTACTACTG | | | | | | | TADESTSTAYMELN | | | | | | |
| | | TGCGAGGGCGGCCGACCCAGGGGTGGGACCTACAATTGGCT | | | | | | | SLTYDDTAVYYCAR | | | | | | |
| | | ACTTCGACCCCTGGGGCCAGGGAACCCTGGTCACCGTCTCC | | | | | | | AADPGVGPTIGYFD | | | | | | |
| | | TCA | | | | | | | PWGQGTLVTVSS | | | | | | |
| ADI-81777 | 1286 | CAGGTGCAGCTGGTGCAGTCTGGGGCTGAAGTGAAGAAGCC | | | | | | 7023 | QVQLVQSGAEVKKP | | 7374 | 18 | | 15 | |
| | | TGGGTCCTCGGTGAAGGTCTCCTGCAAGGCTTCTGGAGGCA | | | | | | | GSSVKVSCKASGGT | | | | | | |
| | | CCTTCAGTAATTATGGTATCAATTGGGTGCGACAGGTCCCT | | | | | | | FSNYGINWVRQVPG | | | | | | |
| | | GGACAGGGGCTTGAGTGGATGGGGAGGATCACCCCTATGTT | | | | | | | QGLEWMGRITPMFG | | | | | | |
| | | CGGGATGGCAAATTATGTGCAGAAATTTCAGGACAGAGTCA | | | | | | | MANYVQKFQDRVTF | | | | | | |
| | | CATTTACCGCGGACAAATCCACGGGCACAGCCTACTTGGAA | | | | | | | TADKSTGTAYLEVS | | | | | | |
| | | GTGAGCAGCCTGACATCTGAGGACACGGCCGTTTATTACTG | | | | | | | SLTSEDTAVYYCAR | | | | | | |
| | | TGCGAGATCGGAGGTGATGAATGTTGGTCATTACTACCTTG | | | | | | | SEVMNVGHYYLDSW | | | | | | |
| | | ACTCCTGGGGCCAGGGAACCCTGGTCACCGTCTCCTCA | | | | | | | GQGTLVTVSS | | | | | | |
| ADI-81728 | 1286 | GAGGTGCAGCTGGTGGAGACGGGCCCAGGACTGGTGAAGCC | | | | | | 7024 | EVQLVETGPGLVKP | | 7375 | 16 | | 13 | |
| | | TTCGCAGACCCTGTCACTCACCTGCAGTGTCTCTGGTGGCT | | | | | | | SQTLSLTCSVSGGS | | | | | | |
| | | CCATCAAGAGCGGTGGTTACTATTGGAGCTGGATCCGCCAG | | | | | | | IKSGGYYWSWIRQH | | | | | | |
| | | CACCCAGGGAAGGGCCTGGAGTGGATTGGCTACATCTATTT | | | | | | | PGKGLEWIGYIYFT | | | | | | |
| | | CACTGGGAGCACCGACTACAACCCGTCCCTCAGAGGTCGAG | | | | | | | GSTDYNPSLRGRVT | | | | | | |
| | | TTACCATTTCAGTGGACACATTTGAGAACCAGTTTTCCTTG | | | | | | | ISVDTFENQFSLRL | | | | | | |
| | | AGGCTGACCTCTGTGACTGCCGCGGACACGGCCACGTATTA | | | | | | | TSVTAADTATYYCA | | | | | | |
| | | CTGTGCGAGAGTTGGTGGTGGTGGGACCAACGACTTTGACT | | | | | | | RVGGGGTNDFDFWG | | | | | | |
| | | TCTGGGGCCAGGGAACCCTGGTCACCGTCTCCTCA | | | | | | | QGTLVTVSS | | | | | | |
| ADI-81732 | 1291 | GAGGTGCAGCTGGTGGAGTCTGGGGGAGACTTGGTACAGCC | | | | | | 7025 | EVQLVESGGDLVQP | | 7376 | 16 | | 15 | |
| | | TGGGGGGTCCCTGAGACTCTCCTGTGCAGCCTCCGGATTCA | | | | | | | GGSLRLSCAASGFT | | | | | | |
| | | CCTTCAGGACCTACGACATGCACTGGGTCCGCCAAACTGCA | | | | | | | FRTYDMHWVRQTAV | | | | | | |
| | | GTAAAAGGTCTGGAGTGGGTCTCCACTATTGGTACTTCAGG | | | | | | | KGLEWVSTIGTSGD | | | | | | |
| | | TGATAGATACTATTCAGACTCCGTGAAGGGCCGATTTACCA | | | | | | | RYYSDSVKGRFTIS | | | | | | |
| | | TCTCCAGAGAGGATGCCAAGGACTCCTTGTATCTTGAAATG | | | | | | | REDAKDSLYLEMNS | | | | | | |
| | | AACAGCCTAAGAGGCGGGGACACGGCTGTGTATTACTGTGC | | | | | | | LRGGDTAVYYCARG | | | | | | |
| | | AAGAGGCAACGATATTGCACTAGTACCACCTGCCATGAGTG | | | | | | | NDIALVPPAMSERP | | | | | | |
| | | AGCGCCCCCGAAACTACTTCTACTACTTGGACGTCTGGGGC | | | | | | | RNYFYYLDVWGKGT | | | | | | |
| | | AAGGGGACCACGGTCACCGTCTCCTCA | | | | | | | TVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81588 | 49 | IGHV3- | QVQLV | 36 | FIFSDY | 227 | WVRQAP | 447 | GISSTG | 550 | RFTISR | 807 | VKDIGSW | 1051 | WGQGTT |
| | | 64D | QSGGG | | SMH | | GKGLDY | | DITDYA | | DNSKNT | | KYFDVFD | | VTVSS |
| | | | LVQPG | | | | VS | | DSVKD | | LYLQMS | | V | | |
| | | | GSLSL | | | | | | | | SLRVED | | | | |
| | | | SCLVS | | | | | | | | TAVYYC | | | | |
| | | | G | | | | | | | | | | | | |
| ADI-81685 | 55 | IGHV3- | EVQLL | 5392 | FTFSNY | 396 | WVRQAP | 5838 | VIWSDG | 6038 | RFTISR | 6354 | ARDYGSR | 6661 | WGQGTL |
| | | 33 | ESGGG | | AMH | | GRGLEW | | TGKHYE | | DNFKNM | | TTWFDP | | VTVSS |
| | | | VVQPG | | | | VP | | DSVRG | | LYLQMN | | | | |
| | | | RSLRL | | | | | | | | SLRVED | | | | |
| | | | SCAVS | | | | | | | | TAVYYC | | | | |
| | | | G | | | | | | | | | | | | |
| ADI-81589 | 50 | IGHV3- | RCTLV | 5443 | VIVSRN | 5665 | WIRQAP | 5839 | VIYSGG | 6039 | RFTISR | 6355 | ARDDARY | 6662 | WGQGTT |
| | | 53 | QSGGG | | YMA | | GRGLEW | | STFYAD | | HNSKNT | | EAFDI | | VTVSS |
| | | | LVQPG | | | | LS | | SVRG | | LYLQMN | | | | |
| | | | GSLRL | | | | | | | | SLRPED | | | | |
| | | | SCTAS | | | | | | | | TAVYYC | | | | |
| | | | E | | | | | | | | | | | | |
| ADI-81592 | 52 | IGHV1- | QVQLV | 5444 | GTISRS | 5666 | WVRQVP | 511 | RIIPML | 6040 | RVTITA | 6356 | ATTNPEL | 6663 | WGQGAL |
| | | 69 | QSGAE | | AIT | | GQGLEW | | GAANYA | | DKSTNT | | SVGGDVF | | VTVSS |
| | | | VKKPG | | | | MG | | QKVQG | | AYMELS | | HV | | |
| | | | TSVKV | | | | | | | | SLRSED | | | | |
| | | | SCKSA | | | | | | | | TAVYYC | | | | |
| | | | G | | | | | | | | | | | | |
| ADI-81751 | 27 | IGHV5- | EVQLV | 5445 | FSFTDY | 5667 | WVRQMP | 5812 | IIFPHD | 6041 | QVTLSA | 6357 | AKGPQLT | 6664 | WGQGTT |
| | | 51 | ESGAE | | WIG | | GKGLEW | | SDTRYS | | DKSINT | | AHPLRVG | | VTVSS |
| | | | VKKPG | | | | VG | | PSFQG | | AYLHWS | | AFDI | | |
| | | | ESLKI | | | | | | | | SLRASD | | | | |
| | | | SCTGS | | | | | | | | TAMYYC | | | | |
| | | | G | | | | | | | | | | | | |
| ADI-81533 | 2 | IGHV3- | RCTLK | 5446 | FTFSND | 5668 | WVRQTP | 5778 | LISYDG | 6042 | RFTISR | 6358 | ARHQESV | 6665 | WGRGTT |
| | | 30 | QSGGG | | DIY | | GKGLEW | | SNSYYA | | DNSNNT | | LPAADRP | | VTVSS |
| | | | VVQPG | | | | VA | | DSVKG | | VYLQMS | | YRYYYYM | | |
| | | | RSLRL | | | | | | | | SLRGED | | DV | | |
| | | | SCAAS | | | | | | | | TAVYYC | | | | |
| | | | G | | | | | | | | | | | | |
| ADI-81548 | 15 | IGHV1- | QVQLQ | 86 | GTFSSY | 407 | WVRQAP | 433 | KIIPIL | 6043 | RVTITA | 6359 | ARESGYS | 6666 | WGRGTL |
| | | 69 | QSGAE | | SIT | | GQGLEW | | NVMDYA | | DKSTGT | | GSGSVVY | | VTVSS |
| | | | VKKPG | | | | MG | | QKFQG | | AYMDLS | | FHL | | |
| | | | SSVKV | | | | | | | | SLTSED | | | | |
| | | | SCKAS | | | | | | | | TAVYYC | | | | |
| | | | G | | | | | | | | | | | | |
| ADI-81582 | 43 | IGHV1- | QVQLV | 50 | DSFSSY | 5669 | WVRQAP | 433 | KIIAFL | 6044 | RVTITA | 6360 | AKEGGTT | 6667 | WGQGTL |
| | | 69 | QSGAE | | TVN | | GQGLEW | | GVSNYA | | DKSTNT | | TEYLQH | | VTVSS |
| | | | VKKPG | | | | MG | | QKFQG | | AYLVLS | | | | |
| | | | SSVKV | | | | | | | | SLRSED | | | | |
| | | | SCKAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81588 | 1287 | CAGGTGCAGCTGGTGCAGTCTGGGGGAGGCTTGGTCCAGCC | | | | | | 1337 | QVQLVQSGGGLVQP | | 1620 | 15 | | 14 | |
| | | TGGGGGGTCCCTGAGCCTCTCCTGTTTAGTCTCTGGATTCA | | | | | | | GGSLSLSCLVSGFI | | | | | | |
| | | TCTTCAGTGACTATTCTATGCACTGGGTCCGCCAGGCTCCA | | | | | | | FSDYSMHWVRQAPG | | | | | | |
| | | GGGAAGGGACTGGATTATGTTTCAGGTATTAGTAGTACTGG | | | | | | | KGLDYVSGISSTGD | | | | | | |
| | | GGATATCACAGACTACGCAGACTCAGTGAAGGACAGATTCA | | | | | | | ITDYADSVKDRFTI | | | | | | |
| | | CCATCTCCAGAGACAATTCCAAGAACACGCTGTATCTTCAA | | | | | | | SRDNSKNTLYLQMS | | | | | | |
| | | ATGAGCAGTCTGAGAGTTGAGGACACGGCTGTGTATTATTG | | | | | | | SLRVEDTAVYYCVK | | | | | | |
| | | TGTGAAAGATATTGGGAGCTGGAAGTATTTTGATGTTTTTG | | | | | | | DIGSWKYFDVFDVW | | | | | | |
| | | ATGTCTGGGGCCAAGGGACCACGGTCACCGTCTCCTCA | | | | | | | GQGTTVTVSS | | | | | | |
| ADI-81685 | 1286 | GAGGTGCAGCTGTTGGAGTCTGGGGGAGGCGTGGTCCAGCC | | | | | | 7026 | EVQLLESGGGVVQP | | 7377 | 15 | | 14 | |
| | | TGGGAGGTCCCTGAGACTCTCCTGTGCAGTGTCTGGATTCA | | | | | | | GRSLRLSCAVSGFT | | | | | | |
| | | CCTTCAGTAACTATGCCATGCACTGGGTCCGCCAGGCTCCA | | | | | | | FSNYAMHWVRQAPG | | | | | | |
| | | GGCAGGGGGTTGGAGTGGGTGCCAGTTATCTGGTCTGATGG | | | | | | | RGLEWVPVIWSDGT | | | | | | |
| | | AACTGGTAAACACTATGAAGACTCAGTGAGGGGCCGGTTCA | | | | | | | GKHYEDSVRGRFTI | | | | | | |
| | | CCATCTCCAGAGACAATTTCAAGAACATGTTGTATCTCCAA | | | | | | | SRDNFKNMLYLQMN | | | | | | |
| | | ATGAACAGCCTGAGAGTCGAGGACACGGCTGTCTATTACTG | | | | | | | SLRVEDTAVYYCAR | | | | | | |
| | | TGCGAGAGATTACGGCAGCAGGACTACCTGGTTCGACCCCT | | | | | | | DYGSRTTWFDPWGQ | | | | | | |
| | | GGGGCCAGGGAACCCTGGTCACCGTCTCCTCA | | | | | | | GTLVTVSS | | | | | | |
| ADI-81589 | 1287 | AGGTGCACGCTGGTGCAGTCTGGAGGAGGCTTGGTCCAGCC | | | | | | 7027 | RCTLVQSGGGLVQP | | 7378 | 14 | | 12 | |
| | | TGGGGGGTCCCTGAGACTCTCCTGCACAGCCTCTGAAGTCA | | | | | | | GGSLRLSCTASEVI | | | | | | |
| | | TCGTCAGTCGCAATTACATGGCCTGGATCCGCCAGGCTCCA | | | | | | | VSRNYMAWIRQAPG | | | | | | |
| | | GGGAGGGGACTGGAGTGGCTCTCAGTTATTTATAGCGGTGG | | | | | | | RGLEWLSVIYSGGS | | | | | | |
| | | TAGCACATTCTACGCAGACTCCGTGAGGGGCCGATTCACCA | | | | | | | TFYADSVRGRFTIS | | | | | | |
| | | TCTCCAGACACAATTCCAAGAACACACTATATCTTCAAATG | | | | | | | RHNSKNTLYLQMNS | | | | | | |
| | | AACAGCCTGAGACCTGAGGACACGGCCGTGTATTACTGTGC | | | | | | | LRPEDTAVYYCARD | | | | | | |
| | | GAGAGATGATGCCCGATATGAAGCTTTTGATATCTGGGGCC | | | | | | | DARYEAFDIWGQGT | | | | | | |
| | | AAGGGACAACGGTCACCGTCTCCTCA | | | | | | | TVTVSS | | | | | | |
| ADI-81592 | 1285 | CAGGTCCAGCTTGTGCAGTCTGGGGCTGAAGTGAAGAAGCC | | | | | | 7028 | QVQLVQSGAEVKKP | | 7379 | 14 | | 12 | |
| | | TGGGACCTCGGTGAAAGTCTCCTGCAAGTCTGCTGGAGGCA | | | | | | | GTSVKVSCKSAGGT | | | | | | |
| | | CCATCAGTAGAAGTGCTATCACCTGGGTGCGACAGGTCCCT | | | | | | | ISRSAITWVRQVPG | | | | | | |
| | | GGACAAGGGCTTGAGTGGATGGGAAGGATCATCCCTATGCT | | | | | | | QGLEWMGRIIPMLG | | | | | | |
| | | TGGCGCAGCAAACTACGCACAGAAGGTCCAGGGCAGAGTCA | | | | | | | AANYAQKVQGRVTI | | | | | | |
| | | CGATTACCGCGGACAAATCCACCAACACAGCCTACATGGAG | | | | | | | TADKSTNTAYMELS | | | | | | |
| | | CTGAGCAGCCTGAGATCTGAGGACACGGCCGTGTATTACTG | | | | | | | SLRSEDTAVYYCAT | | | | | | |
| | | TGCGACAACCAACCCCGAACTCTCAGTGGGTGGAGATGTAT | | | | | | | TNPELSVGGDVFHV | | | | | | |
| | | TTCATGTCTGGGGCCAAGGGGCATTGGTCACCGTCTCTTCA | | | | | | | WGQGALVTVSS | | | | | | |
| ADI-81751 | 1287 | CGGGGAGTCACTGAAGATCTCCTGTACGGGTTCTGGATTCA | | | | | | 7029 | EVQLVESGAEVKKP | | 7380 | 14 | | 10 | |
| | | GCTTTACCGACTACTGGATCGGCTGGGTGCGCCAGATGCCC | | | | | | | GESLKISCTGSGFS | | | | | | |
| | | GGCAAAGGCCTGGAGTGGGTGGGGATCATCTTTCCTCACGA | | | | | | | FTDYWIGWVRQMPG | | | | | | |
| | | CTCTGACACCAGATACAGCCCGTCCTTCCAAGGCCAGGTCA | | | | | | | KGLEWVGIIFPHDS | | | | | | |
| | | CCCTCTCAGCCGACAAGTCCATCAACACCGCCTACCTGCAC | | | | | | | DTRYSPSFQGQVTL | | | | | | |
| | | TGGAGCAGCCTGCGGGCCTCGGACACCGCCATGTATTACTG | | | | | | | SADKSINTAYLHWS | | | | | | |
| | | TGCGAAAGGGCCCCAGCTTACCGCTCATCCCCTGCGGGTCG | | | | | | | SLRASDTAMYYCAK | | | | | | |
| | | GTGCTTTTGATATCTGGGGCCAAGGGACAACGGTCACCGTC | | | | | | | GPQLTAHPLRVGAF | | | | | | |
| | | TCCTCA | | | | | | | DIWGQGTTVTVSS | | | | | | |
| ADI-81533 | 1294 | AGGTGCACGCTGAAGCAGTCTGGGGGAGGCGTGGTCCAGCC | | | | | | 7030 | RCTLKQSGGGVVQP | | 7381 | 13 | | 11 | |
| | | TGGGAGGTCCCTGAGACTCTCCTGTGCAGCCTCTGGATTCA | | | | | | | GRSLRLSCAASGFT | | | | | | |
| | | CCTTCAGTAATGATGATATATACTGGGTCCGCCAGACTCCA | | | | | | | FSNDDIYWVRQTPG | | | | | | |
| | | GGCAAGGGGCTAGAGTGGGTGGCACTTATTTCATACGATGG | | | | | | | KGLEWVALISYDGS | | | | | | |
| | | AAGTAATTCATACTACGCAGACTCCGTGAAGGGCCGGTTCA | | | | | | | NSYYADSVKGRFTI | | | | | | |
| | | CCATCTCCAGAGACAATTCCAACAACACGGTGTATCTGCAA | | | | | | | SRDNSNNTVYLQMS | | | | | | |
| | | ATGAGCAGCCTGAGAGGTGAGGACACGGCTGTATATTACTG | | | | | | | SLRGEDTAVYYCAR | | | | | | |
| | | TGCGAGACATCAAGAGTCCGTACTCCCAGCTGCTGATAGGC | | | | | | | HQESVLPAADRPYR | | | | | | |
| | | CCTATCGTTACTACTACTACATGGACGTCTGGGGCAGAGGG | | | | | | | YYYYMDVWGRGTTV | | | | | | |
| | | ACCACGGTCACCGTCTCCTCA | | | | | | | TVSS | | | | | | |
| ADI-81548 | 1288 | TGGGTCCTCAGTGAAGGTCTCCTGCAAGGCTTCTGGAGGCA | | | | | | 7031 | QVQLQQSGAEVKKP | | 7382 | 13 | | 10 | |
| | | CCTTCAGCAGCTATTCTATCACCTGGGTGCGACAGGCCCCT | | | | | | | GSSVKVSCKASGGT | | | | | | |
| | | GGACAAGGGCTTGAGTGGATGGGGAAGATCATCCCTATCCT | | | | | | | FSSYSITWVRQAPG | | | | | | |
| | | TAATGTAATGGACTACGCACAGAAGTTCCAGGGCAGAGTCA | | | | | | | QGLEWMGKIIPILN | | | | | | |
| | | CGATTACCGCGGACAAATCGACGGGCACAGCCTACATGGAC | | | | | | | VMDYAQKFQGRVTI | | | | | | |
| | | CTGAGCAGCCTGACATCTGAGGACACGGCCGTTTATTACTG | | | | | | | TADKSTGTAYMDLS | | | | | | |
| | | TGCGAGAGAGTCGGGTTATTCTGGTTCGGGCAGTGTTGTAT | | | | | | | SLTSEDTAVYYCAR | | | | | | |
| | | ACTTCCATCTCTGGGGCCGCGGCACCCTGGTCACCGTCTCC | | | | | | | ESGYSGSGSVVYFH | | | | | | |
| | | TCA | | | | | | | LWGRGTLVTVSS | | | | | | |
| ADI-81582 | 1286 | CAGGTGCAGCTGGTGCAGTCTGGGGCTGAGGTGAAGAAGCC | | | | | | 7032 | QVQLVQSGAEVKKP | | 7383 | 13 | | 12 | |
| | | TGGGTCCTCGGTGAAGGTCTCCTGCAAGGCTTCTGGCGACT | | | | | | | GSSVKVSCKASGDS | | | | | | |
| | | CCTTCAGTAGTTATACTGTCAACTGGGTGCGACAGGCCCCT | | | | | | | FSSYTVNWVRQAPG | | | | | | |
| | | GGACAAGGGCTTGAGTGGATGGGGAAGATCATCGCTTTCCT | | | | | | | QGLEWMGKIIAFLG | | | | | | |
| | | TGGAGTGTCAAACTACGCACAGAAGTTCCAGGGCAGAGTCA | | | | | | | VSNYAQKFQGRVTI | | | | | | |
| | | CGATTACCGCGGACAAATCCACGAACACAGCCTACTTGGTG | | | | | | | TADKSTNTAYLVLS | | | | | | |
| | | CTGAGCAGCCTGAGATCTGAGGACACGGCCGTTTACTACTG | | | | | | | SLRSEDTAVYYCAK | | | | | | |
| | | TGCGAAAGAAGGTGGTACCACCACTGAATACCTCCAACACT | | | | | | | EGGTTTEYLQHWGQ | | | | | | |
| | | GGGGCCAGGGCACCCTGGTCACCGTCTCCTCA | | | | | | | GTLVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81541 | 9 | IGHV1- | QVQLE | 5447 | GPFSSS | 5670 | WLRQAP | 478 | RIIPIL | 6045 | RVTITA | 6361 | ARDVGYS | 6668 | WGQGTL |
| | | 69 | QSGAE | | PIS | | GQGLEW | | GVAEYA | | DRSSST | | GSGSCYY | | VTVSS |
| | | | VKKPG | | | | MG | | QKFQG | | AYMELS | | FDP | | |
| | | | SSVKV | | | | | | | | SLRSED | | | | |
| | | | SCKAS | | | | | | | | TALYYC | | | | |
| | | | G | | | | | | | | | | | | |
| ADI-81629 | 2 | IGHV1- | QVQLL | 5448 | GTFDSY | 5671 | WVRQAP | 433 | SIIPSL | 6046 | RVTITA | 6362 | ARGSGYS | 6669 | WGQGTL |
| | | 69 | ESGAE | | TIS | | GQGLEW | | GITDSE | | DKSTST | | GSGSNNY | | VTVSS |
| | | | VKKPG | | | | MG | | QKFQG | | VHMELS | | FDP | | |
| | | | SSVKV | | | | | | | | SLRSED | | | | |
| | | | SCKAS | | | | | | | | TAVYYC | | | | |
| | | | G | | | | | | | | | | | | |
| ADI-81646 | 19 | IGHV4- | QVQLL | 5449 | GSISSN | 5672 | WIRQPP | 445 | FVFDSG | 6047 | RITISI | 6363 | ARHGPDS | 6670 | WGQGTT |
| | | 4 | ESGPG | | YWS | | GKGLEW | | STNYNP | | DTSRNQ | | SSRYHDA | | VTVSS |
| | | | LVKPS | | | | IG | | SLRS | | FSLKLR | | FDI | | |
| | | | ETLSL | | | | | | | | SVTAAD | | | | |
| | | | TCSVS | | | | | | | | TAVYYC | | | | |
| | | | G | | | | | | | | | | | | |
| ADI-81745 | 21 | IGHV1- | QVQLV | 5450 | YTFSNY | 5673 | WVRQAP | 5840 | MINPSG | 6048 | RVIMTG | 6364 | ARSQVVP | 1238 | WGQGTL |
| | | 46 | ESGAE | | YVH | | GHGLEW | | GSTTYA | | DTSTKT | | ATSHLDY | | VTVSS |
| | | | VKKPG | | | | MG | | QKFQG | | VYMELS | | | | |
| | | | ASVKI | | | | | | | | SLRSED | | | | |
| | | | SCKAS | | | | | | | | TAVYSC | | | | |
| | | | G | | | | | | | | | | | | |
| ADI-81630 | 3 | IGHV1- | QVQLV | 5451 | GTFSSY | 407 | WVRQAP | 433 | TIIPIL | 6049 | RVTITA | 987 | AREYCSG | 1248 | WGQGTL |
| | | 69 | QSGAE | | SIT | | GQGLEW | | GVPNYA | | DKSTST | | DSCRTVN | | VTVSS |
| | | | VKEPG | | | | MG | | QKFQG | | AYMEVT | | WFDP | | |
| | | | SSVKV | | | | | | | | SLTSED | | | | |
| | | | SCKAS | | | | | | | | TAIYYC | | | | |
| | | | G | | | | | | | | | | | | |
| ADI-81660 | 2 | IGHV1- | QVQLV | 5452 | GAFSSY | 5674 | WVRQAP | 433 | RVIAFL | 6050 | RVTITA | 6365 | ARDSGYS | 6671 | WGQGNA |
| | | 69 | ESGAE | | SIS | | GQGLEW | | NIPNYA | | DKSTST | | GSGSNNY | | VTISS |
| | | | VKKHG | | | | MG | | QKFQG | | AYMELT | | FDP | | |
| | | | SSVKV | | | | | | | | SLRSED | | | | |
| | | | SCKAS | | | | | | | | TAVYYC | | | | |
| | | | A | | | | | | | | | | | | |
| ADI-81733 | 10 | IGHV4- | EVQLL | 5453 | GSTRNY | 5675 | WIRQPP | 445 | YIDYSG | 6051 | RVTISI | 6366 | ARVGRAD | 6672 | WGKGTT |
| | | 59 | ESGPG | | YWT | | GKGLEW | | STNYNL | | DTAKNQ | | AVVPAGI | | VTVSS |
| | | | LVKPS | | | | IG | | SLES | | FSLRLS | | DYYYYMD | | |
| | | | ETLSL | | | | | | | | SVTAAD | | V | | |
| | | | SCTVS | | | | | | | | TAVYYC | | | | |
| | | | G | | | | | | | | | | | | |
| ADI-81736 | 13 | IGHV3- | EVQLL | 5373 | FTFGSY | 5676 | WVRQAP | 5841 | FIPYDG | 6052 | RFTVSR | 894 | ATEAGFD | 6673 | WGKGTT |
| | | 33 | ESGGG | | GMH | | GKGLDW | | RNPHYA | | DNSKNT | | PNYNYYL | | VTVSS |
| | | | VVQPG | | | | VA | | DSVKG | | LYLQMN | | DV | | |
| | | | RSLRL | | | | | | | | SLRAED | | | | |
| | | | SCAAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81541 | 1286 | TGGGTCCTCGGTGAAGGTCTCCTGCAAGGCTTCTGGAGGCC | | | | | | 7033 | QVQLEQSGAEVKKP | | 7384 | 12 | | 9 | |
| | | CCTTCAGCAGTTCTCCAATCAGCTGGCTGCGGCAGGCCCCT | | | | | | | GSSVKVSCKASGGP | | | | | | |
| | | GGACAGGGGCTTGAGTGGATGGGAAGGATCATCCCTATCCT | | | | | | | FSSSPISWLRQAPG | | | | | | |
| | | TGGTGTGGCAGAATACGCACAGAAGTTTCAGGGCAGAGTCA | | | | | | | QGLEWMGRIIPILG | | | | | | |
| | | CCATTACCGCGGACAGATCCTCGAGCACAGCCTACATGGAG | | | | | | | VAEYAQKFQGRVTI | | | | | | |
| | | CTGAGCAGCCTGAGATCTGAGGACACGGCCCTGTATTATTG | | | | | | | TADRSSSTAYMELS | | | | | | |
| | | TGCGAGAGATGTGGGTTACTCTGGTTCGGGGAGTTGTTACT | | | | | | | SLRSEDTALYYCAR | | | | | | |
| | | ACTTTGACCCCTGGGGCCAGGGAACCCTGGTCACCGTCTCC | | | | | | | DVGYSGSGSCYYFD | | | | | | |
| | | TCA | | | | | | | PWGQGTLVTVSS | | | | | | |
| ADI-81629 | 1286 | TGGGTCCTCGGTGAAGGTCTCCTGCAAGGCCTCTGGAGGCA | | | | | | 7034 | QVQLLESGAEVKKP | | 7385 | 12 | | 9 | |
| | | CCTTCGACAGCTATACTATCAGCTGGGTGCGACAGGCCCCT | | | | | | | GSSVKVSCKASGGT | | | | | | |
| | | GGACAAGGGCTTGAGTGGATGGGAAGCATCATTCCTTCCCT | | | | | | | FDSYTISWVRQAPG | | | | | | |
| | | TGGTATAACAGACTCCGAACAGAAGTTCCAGGGCAGAGTCA | | | | | | | QGLEWMGSIIPSLG | | | | | | |
| | | CGATTACCGCGGACAAATCCACGAGCACAGTTCACATGGAG | | | | | | | ITDSEQKFQGRVTI | | | | | | |
| | | CTGAGCAGCCTGAGATCTGAGGACACGGCCGTGTATTACTG | | | | | | | TADKSTSTVHMELS | | | | | | |
| | | TGCGAGAGGTTCGGGGTATTCTGGTTCAGGGAGTAACAACT | | | | | | | SLRSEDTAVYYCAR | | | | | | |
| | | ACTTCGACCCCTGGGGCCAGGGAACCCTGGTCACCGTCTCC | | | | | | | GSGYSGSGSNNYFD | | | | | | |
| | | TCA | | | | | | | PWGQGTLVTVSS | | | | | | |
| ADI-81646 | 1287 | CAGGTGCAGCTGTTGGAGTCGGGCCCAGGACTGGTGAAGCC | | | | | | 7035 | QVQLLESGPGLVKP | | 7386 | 12 | | 11 | |
| | | TTCGGAGACCCTGTCCCTCACCTGCAGTGTCTCTGGTGGCT | | | | | | | SETLSLTCSVSGGS | | | | | | |
| | | CCATCAGTAGTAACTACTGGAGCTGGATCCGGCAGCCCCCA | | | | | | | ISSNYWSWIRQPPG | | | | | | |
| | | GGGAAGGGACTGGAGTGGATTGGGTTTGTCTTTGACAGTGG | | | | | | | KGLEWIGFVFDSGS | | | | | | |
| | | GAGCACCAACTACAACCCCTCCCTCAGGAGTCGAATCACCA | | | | | | | TNYNPSLRSRITIS | | | | | | |
| | | TATCAATAGATACGTCCAGGAACCAGTTCTCCCTGAAGCTG | | | | | | | IDTSRNQFSLKLRS | | | | | | |
| | | AGGTCTGTGACCGCCGCAGACACGGCCGTGTACTACTGTGC | | | | | | | VTAADTAVYYCARH | | | | | | |
| | | GAGACATGGGCCCGATAGCAGCAGCAGGTACCATGATGCTT | | | | | | | GPDSSSRYHDAFDI | | | | | | |
| | | TTGATATCTGGGGCCAAGGGACCACGGTCACCGTCTCCTCA | | | | | | | WGQGTTVTVSS | | | | | | |
| ADI-81745 | 1286 | CAGGTGCAGCTGGTGGAGTCTGGGGCTGAGGTGAAGAAGCC | | | | | | 7036 | QVQLVESGAEVKKP | | 7387 | 12 | | 11 | |
| | | TGGGGCCTCAGTGAAGATTTCCTGCAAGGCATCTGGATACA | | | | | | | GASVKISCKASGYT | | | | | | |
| | | CTTTCAGCAACTACTATGTGCACTGGGTGCGACAGGCCCCT | | | | | | | FSNYYVHWVRQAPG | | | | | | |
| | | GGACACGGGCTTGAGTGGATGGGAATGATCAACCCTAGTGG | | | | | | | HGLEWMGMINPSGG | | | | | | |
| | | TGGTAGCACAACCTACGCACAGAAATTCCAGGGCAGAGTCA | | | | | | | STTYAQKFQGRVIM | | | | | | |
| | | TCATGACCGGGGACACGTCGACGAAAACAGTGTACATGGAA | | | | | | | TGDTSTKTVYMELS | | | | | | |
| | | CTGAGCAGCCTGAGGTCTGAGGACACGGCCGTTTATTCCTG | | | | | | | SLRSEDTAVYSCAR | | | | | | |
| | | TGCGAGGAGCCAAGTAGTACCAGCTACTAGTCACCTTGACT | | | | | | | SQVVPATSHLDYWG | | | | | | |
| | | ACTGGGGCCAGGGAACCCTGGTCACCGTCTCCTCA | | | | | | | QGTLVTVSS | | | | | | |
| ADI-81630 | 1286 | TGGGTCCTCGGTGAAGGTCTCCTGCAAGGCTTCTGGAGGCA | | | | | | 7037 | QVQLVQSGAEVKEP | | 7388 | 11 | | 10 | |
| | | CCTTCAGCAGCTATAGTATCACCTGGGTGCGACAGGCCCCT | | | | | | | GSSVKVSCKASGGT | | | | | | |
| | | GGACAAGGGCTTGAGTGGATGGGAACGATCATCCCTATCCT | | | | | | | FSSYSITWVRQAPG | | | | | | |
| | | TGGTGTACCAAACTACGCACAGAAGTTCCAGGGCAGAGTCA | | | | | | | QGLEWMGTIIPILG | | | | | | |
| | | CCATTACCGCGGACAAATCCACGAGCACAGCCTACATGGAG | | | | | | | VPNYAQKFQGRVTI | | | | | | |
| | | GTGACCAGCCTGACATCTGAGGACACGGCCATATATTACTG | | | | | | | TADKSTSTAYMEVT | | | | | | |
| | | TGCGAGAGAATATTGTAGTGGTGACAGCTGCCGAACCGTTA | | | | | | | SLTSEDTAIYYCAR | | | | | | |
| | | ACTGGTTCGACCCCTGGGGCCAGGGAACCCTGGTCACCGTC | | | | | | | EYCSGDSCRTVNWF | | | | | | |
| | | TCCTCA | | | | | | | DPWGQGTLVTVSS | | | | | | |
| ADI-81660 | 6821 | TGGGTCCTCGGTGAAGGTCTCCTGCAAGGCTTCTGCAGGCG | | | | | | 7038 | QVQLVESGAEVKKH | | 7389 | 11 | | 10 | |
| | | CCTTCAGCAGCTACTCTATCAGCTGGGTGCGACAGGCCCCT | | | | | | | GSSVKVSCKASAGA | | | | | | |
| | | GGACAAGGGCTTGAGTGGATGGGAAGAGTCATCGCTTTCCT | | | | | | | FSSYSISWVRQAPG | | | | | | |
| | | TAATATACCAAACTACGCACAGAAATTCCAGGGCAGAGTCA | | | | | | | QGLEWMGRVIAFLN | | | | | | |
| | | CCATTACCGCGGACAAATCCACGAGTACAGCCTACATGGAG | | | | | | | IPNYAQKFQGRVTI | | | | | | |
| | | CTGACCAGCCTCAGATCTGAGGACACGGCCGTGTATTATTG | | | | | | | TADKSTSTAYMELT | | | | | | |
| | | TGCGAGAGATTCGGGGTACTCTGGTTCAGGGAGTAACAACT | | | | | | | SLRSEDTAVYYCAR | | | | | | |
| | | ACTTCGACCCCTGGGGCCAGGGTAACGCGGTCACAATTTCA | | | | | | | DSGYSGSGSNNYFD | | | | | | |
| | | TCG | | | | | | | PWGQGNAVTISS | | | | | | |
| ADI-81733 | 1291 | GAGGTGCAGCTGTTGGAGTCGGGCCCAGGCCTGGTGAAGCC | | | | | | 7039 | EVQLLESGPGLVKP | | 7390 | 11 | | 11 | |
| | | GTCGGAGACCCTGTCCCTCTCCTGCACTGTCTCTGGTGGCT | | | | | | | SETLSLSCTVSGGS | | | | | | |
| | | CCACCAGAAATTACTACTGGACCTGGATCCGGCAGCCCCCA | | | | | | | TRNYYWTWIRQPPG | | | | | | |
| | | GGGAAGGGACTGGAGTGGATTGGGTATATCGATTACAGTGG | | | | | | | KGLEWIGYIDYSGS | | | | | | |
| | | TAGCACCAACTACAACCTCTCCCTGGAGAGTCGAGTCACCA | | | | | | | TNYNLSLESRVTIS | | | | | | |
| | | TATCAATAGACACGGCCAAGAACCAGTTCTCCCTGAGGCTG | | | | | | | IDTAKNQFSLRLSS | | | | | | |
| | | AGCTCTGTGACCGCTGCGGACACGGCCGTATATTACTGTGC | | | | | | | VTAADTAVYYCARV | | | | | | |
| | | GAGAGTCGGGAGGGCTGATGCAGTAGTACCAGCTGGTATAG | | | | | | | GRADAVVPAGIDYY | | | | | | |
| | | ATTATTACTACTACATGGATGTCTGGGGCAAAGGGACCACG | | | | | | | YYMDVWGKGTTVTV | | | | | | |
| | | GTCACCGTCTCCTCA | | | | | | | SS | | | | | | |
| ADI-81736 | 1291 | GAGGTGCAGCTGTTGGAGTCTGGGGGAGGCGTGGTCCAGCC | | | | | | 7040 | EVQLLESGGGVVQP | | 7391 | 11 | | 8 | |
| | | AGGGAGGTCCCTGAGACTCTCCTGTGCAGCGTCTGGATTCA | | | | | | | GRSLRLSCAASGFT | | | | | | |
| | | CCTTCGGGAGCTATGGCATGCACTGGGTCCGCCAGGCTCCA | | | | | | | FGSYGMHWVRQAPG | | | | | | |
| | | GGCAAGGGGCTGGATTGGGTGGCATTTATACCGTATGATGG | | | | | | | KGLDWVAFIPYDGR | | | | | | |
| | | AAGGAATCCACACTATGCCGACTCCGTGAAGGGCCGATTCA | | | | | | | NPHYADSVKGRFTV | | | | | | |
| | | CCGTCTCCAGAGACAACTCCAAAAACACGCTGTATCTGCAG | | | | | | | SRDNSKNTLYLQMN | | | | | | |
| | | ATGAACAGCTTGAGAGCCGAGGACACGGCTGTCTACTACTG | | | | | | | SLRAEDTAVYYCAT | | | | | | |
| | | TGCGACAGAGGCGGGATTTGACCCCAACTACAACTACTACT | | | | | | | EAGFDPNYNYYLDV | | | | | | |
| | | TGGACGTCTGGGGCAAAGGGACCACGGTCACCGTCTCCTCA | | | | | | | WGKGTTVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81759 | 35 | IGHV4- | EVQLV | 5347 | GSISGP | 5677 | WVRQPP | 444 | EIFHTG | 6053 | RATISI | 6367 | ARIMGAL | 6674 | WGQGTL |
| | | 4 | ESGPG | | NWWS | | GKGLEW | | NTKYNP | | DKSKNQ | | DY | | VTVSS |
| | | | LVKPS | | | | IG | | SLKS | | FSLKLS | | | | |
| | | | GTLSL | | | | | | | | SVTAAD | | | | |
| | | | TCAVS | | | | | | | | TAIYYC | | | | |
| | | | G | | | | | | | | | | | | |
| ADI-81542 | 10 | IGHV3- | QVQLV | 12 | FTFSSY | 207 | WVRQDT | 5842 | VIGTYG | 6054 | RFTISR | 6368 | ARGTSVT | 6675 | WGKGTT |
| | | 13 | QSGGG | | DMH | | GKRLEW | | DTYYPD | | ENGKNS | | RGSYHYY | | VTVSS |
| | | | LVQPG | | | | VS | | SVRG | | LFLQMN | | YMDV | | |
| | | | GSLRL | | | | | | | | SLRAGD | | | | |
| | | | SCAAS | | | | | | | | TAVYFC | | | | |
| | | | G | | | | | | | | | | | | |
| ADI-81639 | 12 | IGHV3- | QVQLL | 5454 | FTFDDF | 5678 | WVRQAP | 437 | GINWNG | 6055 | RFTIFR | 6369 | VRSYDTG | 6676 | WGSGTT |
| | | 20 | ESGGR | | AMS | | GKGLEW | | AGAGYA | | DNAKNF | | DV | | VTVSS |
| | | | VVRPG | | | | VS | | DSVKG | | LYLEMN | | | | |
| | | | GSLRL | | | | | | | | SLRVED | | | | |
| | | | SCAAS | | | | | | | | TALYHC | | | | |
| | | | G | | | | | | | | | | | | |
| ADI-81815 | 89 | IGHV4- | EVQLL | 5335 | GSISRT | 5679 | WIRQSP | 434 | NIFYSG | 6056 | RLTISL | 6370 | TRQIGMA | 6677 | WGQGTL |
| | | 39 | ESGPG | | TYYWG | | GKGLEW | | TTYYNP | | DTSKNQ | | DY | | VTVSS |
| | | | LVKPS | | | | IG | | SLKS | | FSLKLT | | | | |
| | | | ETLSL | | | | | | | | SVTAAD | | | | |
| | | | TCTVS | | | | | | | | TAVYYC | | | | |
| | | | G | | | | | | | | | | | | |
| ADI-81852 | 30 | IGHV1- | EVQLV | 5320 | YSFTNY | 5680 | WVRQAP | 433 | IINAGS | 6057 | RVTMTR | 6371 | ARDPGVV | 6678 | WGQGTL |
| | | 46 | QSGAE | | YIH | | GQGLEW | | GGTSYP | | DTSTST | | DPLNPHP | | VTVSS |
| | | | VKKPG | | | | MG | | QKFQG | | VDMELS | | LPRF | | |
| | | | ASVKV | | | | | | | | SLRSED | | | | |
| | | | SCKAS | | | | | | | | TAVYYC | | | | |
| | | | G | | | | | | | | | | | | |
| ADI-81853 | 31 | IGHV1- | EVQLV | 5455 | HTFSTY | 5681 | WVRQAP | 433 | IIHPSG | 6058 | RVTMTS | 6372 | ARGGLVP | 6679 | WGQGTT |
| | | 46 | ESGAE | | YIH | | GQGLEW | | GSTTYA | | DSSTST | | DAQEPFD | | VTVSS |
| | | | VKKPG | | | | MG | | QKFQG | | VYMDLS | | I | | |
| | | | ASVRV | | | | | | | | SLRSED | | | | |
| | | | SCKAS | | | | | | | | TAVYYC | | | | |
| | | | G | | | | | | | | | | | | |
| ADI-81647 | 20 | IGHV1- | QVQLV | 5456 | GSFSSY | 5682 | WVRQAP | 433 | RIIPFL | 6059 | RVTITA | 6373 | ARGSGYS | 6680 | WGQGTL |
| | | 69 | QSGAE | | TIS | | GQGLEW | | GVADLA | | DKSTNT | | SGGSNNY | | VTVSS |
| | | | LKKPG | | | | MG | | QKFQG | | ASMELS | | FDS | | |
| | | | SSVKV | | | | | | | | SLRSED | | | | |
| | | | SCKAS | | | | | | | | TAVYYC | | | | |
| | | | G | | | | | | | | | | | | |
| ADI-81689 | 59 | IGHV1- | EVQLV | 5320 | YTFTSF | 5683 | WVRQAP | 433 | WISTFN | 6060 | RVTLTT | 6374 | AREGPFY | 6681 | WGQGTL |
| | | 18 | QSGAE | | GVN | | GQGLEW | | GNTNFA | | DTSTST | | CSGGSCY | | VTVSS |
| | | | VKKPG | | | | MG | | QKFQG | | VYMELR | | FFDY | | |
| | | | ASVKV | | | | | | | | SLRSDD | | | | |
| | | | SCKAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81759 | 1286 | GAGGTGCAGCTGGTGGAGTCGGGCCCAGGACTGGTGAAGCC | | | | | | 7041 | EVQLVESGPGLVKP | | 7392 | 11 | | 9 | |
| | | TTCGGGGACCCTGTCCCTCACCTGCGCTGTCTCTGGTGGCT | | | | | | | SGTLSLTCAVSGGS | | | | | | |
| | | CCATCAGCGGTCCTAATTGGTGGAGTTGGGTCCGCCAGCCC | | | | | | | ISGPNWWSWVRQPP | | | | | | |
| | | CCAGGGAAGGGGCTGGAGTGGATTGGGGAAATCTTTCATAC | | | | | | | GKGLEWIGEIFHTG | | | | | | |
| | | TGGGAACACCAAGTACAACCCGTCCCTCAAGAGTCGAGCCA | | | | | | | NTKYNPSLKSRATI | | | | | | |
| | | CCATATCAATAGACAAGTCCAAGAACCAGTTCTCCCTGAAG | | | | | | | SIDKSKNQFSLKLS | | | | | | |
| | | CTGAGTTCTGTGACCGCCGCGGACACGGCCATTTATTACTG | | | | | | | SVTAADTAIYYCAR | | | | | | |
| | | TGCGAGAATAATGGGAGCCCTTGACTACTGGGGGCAGGGAA | | | | | | | IMGALDYWGQGTLV | | | | | | |
| | | CCCTGGTCACCGTCTCCTCA | | | | | | | TVSS | | | | | | |
| ADI-81542 | 1291 | TGGGGGGTCCCTGAGACTCTCCTGTGCAGCCTCTGGATTCA | | | | | | 7042 | QVQLVQSGGGLVQP | | 7393 | 10 | | 9 | |
| | | CCTTCAGTAGCTACGACATGCACTGGGTCCGCCAAGATACA | | | | | | | GGSLRLSCAASGFT | | | | | | |
| | | GGAAAACGTCTAGAATGGGTCTCAGTTATTGGTACTTATGG | | | | | | | FSSYDMHWVRQDTG | | | | | | |
| | | TGACACATACTATCCAGACTCCGTGAGGGGCCGATTCACCA | | | | | | | KRLEWVSVIGTYGD | | | | | | |
| | | TCTCCAGAGAAAATGGCAAGAACTCCTTGTTTCTTCAAATG | | | | | | | TYYPDSVRGRFTIS | | | | | | |
| | | AACAGCCTGAGAGCCGGGGACACGGCTGTCTATTTCTGTGC | | | | | | | RENGKNSLFLQMNS | | | | | | |
| | | AAGAGGGACGTCGGTCACCCGTGGGTCCTACCACTACTACT | | | | | | | LRAGDTAVYFCARG | | | | | | |
| | | ACATGGACGTCTGGGGCAAAGGGACCACGGTCACCGTCTCC | | | | | | | TSVTRGSYHYYYMD | | | | | | |
| | | TCA | | | | | | | VWGKGTTVTVSS | | | | | | |
| ADI-81639 | 6822 | CAGGTGCAGCTGTTGGAGTCTGGGGGACGTGTGGTACGGCC | | | | | | 7043 | QVQLLESGGRVVRP | | 7394 | 10 | | 10 | |
| | | GGGGGGGTCCCTGAGACTCTCCTGTGCAGCCTCTGGATTCA | | | | | | | GGSLRLSCAASGFT | | | | | | |
| | | CCTTTGATGATTTTGCCATGAGTTGGGTCCGCCAAGCTCCA | | | | | | | FDDFAMSWVRQAPG | | | | | | |
| | | GGGAAGGGACTGGAGTGGGTCTCTGGGATTAACTGGAATGG | | | | | | | KGLEWVSGINWNGA | | | | | | |
| | | TGCTGGCGCAGGGTATGCAGACTCTGTGAAGGGCCGATTCA | | | | | | | GAGYADSVKGRFTI | | | | | | |
| | | CCATCTTCAGAGACAACGCCAAGAACTTCCTGTATCTGGAA | | | | | | | FRDNAKNFLYLEMN | | | | | | |
| | | ATGAACAGTCTGAGAGTCGAAGACACGGCCCTCTATCACTG | | | | | | | SLRVEDTALYHCVR | | | | | | |
| | | TGTGAGAAGCTACGATACCGGGGACGTCTGGGGCAGCGGGA | | | | | | | SYDTGDVWGSGTTV | | | | | | |
| | | CCACGGTCACCGTCTCCTCA | | | | | | | TVSS | | | | | | |
| ADI-81815 | 1286 | GAGGTGCAGCTGTTGGAGTCGGGCCCAGGACTGGTGAAGCC | | | | | | 7044 | EVQLLESGPGLVKP | | 7395 | 10 | | 10 | |
| | | TTCGGAGACCCTGTCCCTCACCTGCACTGTCTCTGGTGGCT | | | | | | | SETLSLTCTVSGGS | | | | | | |
| | | CCATCAGCAGGACTACTTACTACTGGGGCTGGATCCGCCAG | | | | | | | ISRTTYYWGWIRQS | | | | | | |
| | | TCCCCCGGGAAGGGACTAGAATGGATTGGGAATATCTTTTA | | | | | | | PGKGLEWIGNIFYS | | | | | | |
| | | TAGTGGGACCACCTACTACAACCCGTCCCTCAAGAGTCGAC | | | | | | | GTTYYNPSLKSRLT | | | | | | |
| | | TCACCATATCCCTAGACACGTCCAAGAACCAGTTCTCCCTG | | | | | | | ISLDTSKNQFSLKL | | | | | | |
| | | AAACTGACCTCTGTGACCGCCGCAGACACGGCTGTCTATTA | | | | | | | TSVTAADTAVYYCT | | | | | | |
| | | CTGTACAAGACAGATTGGTATGGCAGACTACTGGGGCCAGG | | | | | | | RQIGMADYWGQGTL | | | | | | |
| | | GAACCCTGGTCACCGTCTCCTCA | | | | | | | VTVSS | | | | | | |
| ADI-81852 | 1286 | TGGGGCCTCAGTGAAGGTTTCCTGCAAGGCGTCTGGATACA | | | | | | 7045 | EVQLVQSGAEVKKP | | 7396 | 10 | | 9 | |
| | | GCTTCACCAACTACTATATACACTGGGTGCGACAGGCCCCT | | | | | | | GASVKVSCKASGYS | | | | | | |
| | | GGACAAGGGCTTGAGTGGATGGGAATAATCAATGCTGGTAG | | | | | | | FTNYYIHWVRQAPG | | | | | | |
| | | TGGTGGCACAAGCTACCCTCAGAAGTTCCAGGGCCGAGTCA | | | | | | | QGLEWMGIINAGSG | | | | | | |
| | | CCATGACCAGGGACACGTCCACGAGCACAGTCGACATGGAG | | | | | | | GTSYPQKFQGRVTM | | | | | | |
| | | CTGAGTAGCCTGAGATCTGAGGACACGGCCGTGTATTACTG | | | | | | | TRDTSTSTVDMELS | | | | | | |
| | | TGCGAGAGATCCGGGTGTGGTGGACCCGCTGAATCCCCACC | | | | | | | SLRSEDTAVYYCAR | | | | | | |
| | | CCCTGCCTCGTTTCTGGGGCCAGGGAACCCTGGTCACCGTC | | | | | | | DPGVVDPLNPHPLP | | | | | | |
| | | TCCTCA | | | | | | | RFWGQGTLVTVSS | | | | | | |
| ADI-81853 | 1287 | GAGGTGCAGCTGGTGGAGTCTGGGGCTGAGGTGAAGAAGCC | | | | | | 7046 | EVQLVESGAEVKKP | | 7397 | 10 | | 10 | |
| | | TGGGGCCTCGGTGAGGGTTTCCTGCAAGGCATCTGGACACA | | | | | | | GASVRVSCKASGHT | | | | | | |
| | | CCTTCAGCACCTACTATATTCACTGGGTGCGACAGGCCCCT | | | | | | | FSTYYIHWVRQAPG | | | | | | |
| | | GGACAAGGGCTTGAGTGGATGGGAATAATCCACCCTAGTGG | | | | | | | QGLEWMGIIHPSGG | | | | | | |
| | | TGGTAGCACAACCTACGCACAGAAGTTCCAGGGCAGAGTCA | | | | | | | STTYAQKFQGRVTM | | | | | | |
| | | CCATGACCAGCGACTCGTCCACGAGCACAGTCTACATGGAC | | | | | | | TSDSSTSTVYMDLS | | | | | | |
| | | TTGAGCAGCCTGAGATCTGAGGACACGGCCGTGTATTACTG | | | | | | | SLRSEDTAVYYCAR | | | | | | |
| | | TGCGAGAGGGGGATTAGTTCCAGATGCGCAAGAGCCTTTTG | | | | | | | GGLVPDAQEPFDIW | | | | | | |
| | | ATATCTGGGGCCAGGGGACCACGGTCACCGTCTCCTCA | | | | | | | GQGTTVTVSS | | | | | | |
| ADI-81647 | 1286 | TGGGTCCTCGGTGAAGGTCTCCTGCAAGGCTTCTGGAGGCT | | | | | | 7047 | QVQLVQSGAELKKP | | 7398 | 9 | | 8 | |
| | | CCTTCAGCAGTTATACTATCAGCTGGGTGCGACAGGCCCCT | | | | | | | GSSVKVSCKASGGS | | | | | | |
| | | GGACAAGGCCTTGAGTGGATGGGAAGGATCATCCCTTTCCT | | | | | | | FSSYTISWVRQAPG | | | | | | |
| | | TGGTGTAGCAGACCTCGCACAGAAGTTCCAGGGCCGAGTCA | | | | | | | QGLEWMGRIIPFLG | | | | | | |
| | | CGATTACCGCGGACAAGTCCACGAACACAGCCTCCATGGAG | | | | | | | VADLAQKFQGRVTI | | | | | | |
| | | TTGAGCAGCCTGAGATCTGAAGACACGGCCGTGTATTACTG | | | | | | | TADKSTNTASMELS | | | | | | |
| | | TGCGAGAGGCTCCGGCTATAGCAGCGGAGGTAGTAATAACT | | | | | | | SLRSEDTAVYYCAR | | | | | | |
| | | ACTTCGACTCCTGGGGCCAGGGAACCCTGGTCACCGTCTCC | | | | | | | GSGYSSGGSNNYFD | | | | | | |
| | | TCA | | | | | | | SWGQGTLVTVSS | | | | | | |
| ADI-81689 | 1286 | TGGGGCCTCAGTGAAGGTCTCCTGCAAGGCTTCTGGTTACA | | | | | | 7048 | EVQLVQSGAEVKKP | | 7399 | 9 | | 9 | |
| | | CCTTTACCAGTTTTGGTGTCAACTGGGTGCGACAGGCCCCT | | | | | | | GASVKVSCKASGYT | | | | | | |
| | | GGACAAGGCCTTGAGTGGATGGGATGGATCAGCACTTTCAA | | | | | | | FTSFGVNWVRQAPG | | | | | | |
| | | TGGTAATACAAACTTTGCACAGAAGTTCCAGGGCAGAGTCA | | | | | | | QGLEWMGWISTFNG | | | | | | |
| | | CCCTGACCACAGACACATCCACGAGCACAGTCTACATGGAG | | | | | | | NTNFAQKFQGRVTL | | | | | | |
| | | CTGCGGAGCCTGAGATCTGACGACACGGCCGTGTATTATTG | | | | | | | TTDTSTSTVYMELR | | | | | | |
| | | TGCGCGAGAGGGGCCTTTTTATTGTAGTGGTGGTAGTTGCT | | | | | | | SLRSDDTAVYYCAR | | | | | | |
| | | ACTTTTTTGACTACTGGGGCCAGGGAACCCTGGTCACCGTC | | | | | | | EGPFYCSGGSCYFF | | | | | | |
| | | TCCTCA | | | | | | | DYWGQGTLVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81550 | 17 | IGHV1- | QVQLV | 5457 | GTFSSY | 5684 | WVRQAP | 433 | RIIPIL | 6061 | RVTITA | 6375 | AREAGYS | 6682 | WSQGTL |
| | | 69 | QSGAE | | TVS | | GQGLEW | | GIANYA | | DKSTAT | | GSGSSYY | | VTVSS |
| | | | VKKPG | | | | MG | | QNFQG | | VYLEVS | | FDY | | |
| | | | SSVKV | | | | | | | | SLRSED | | | | |
| | | | SCMAS | | | | | | | | TAVYYC | | | | |
| | | | G | | | | | | | | | | | | |
| ADI-81552 | 17 | IGHV1- | QVQLV | 50 | GTFSSN | 5685 | WVRQAP | 433 | RIIPIL | 6062 | RVTITA | 6376 | AREAGYS | 6682 | WSQGTL |
| | | 69 | QSGAE | | TVS | | GQGLEW | | GIADYA | | DKSTAT | | GSGSSYY | | VTVSS |
| | | | VKKPG | | | | MG | | QKFQG | | VYMEVS | | FDY | | |
| | | | SSVKV | | | | | | | | SLRSED | | | | |
| | | | SCKAS | | | | | | | | TAMYYC | | | | |
| | | | G | | | | | | | | | | | | |
| ADI-81601 | 15 | IGHV1- | QVQLV | 50 | GTFSSY | 356 | WVRQAP | 495 | KIIPIL | 6063 | RVTIIA | 936 | ARESGYS | 1191 | WGRGTL |
| | | 69 | QSGAE | | SIS | | GQGPEW | | GATDYA | | DKSTST | | GSGSVTY | | VTVSS |
| | | | VKKPG | | | | LG | | QKFQG | | AYMELS | | FHL | | |
| | | | SSVKV | | | | | | | | SLRSED | | | | |
| | | | SCKAS | | | | | | | | TAVYYC | | | | |
| | | | G | | | | | | | | | | | | |
| ADI-81653 | 26 | IGHV3- | EVQLL | 5458 | FIFSDY | 5686 | WVRQAP | 431 | VIWYDG | 6064 | RFTISR | 779 | ARLATVP | 6683 | WGKGTT |
| | | 33 | ESGGG | | GIH | | GKGLEW | | SNTYYA | | DNSKNT | | YFYYMDV | | VTVSS |
| | | | VIQPG | | | | VA | | DSVKG | | VYLQMN | | | | |
| | | | RSLRL | | | | | | | | SLRAED | | | | |
| | | | SCAAS | | | | | | | | TAVYYC | | | | |
| | | | G | | | | | | | | | | | | |
| ADI-81661 | 33 | IGHV1- | EVQLV | 5459 | GTFSSY | 252 | WVRQAP | 433 | RIIPIL | 6065 | RVTITA | 6377 | ATEVGYS | 6684 | WGQGTL |
| | | 69 | ESGAE | | TIT | | GQGLEW | | GIPNYA | | DKSTSM | | GYGPSPY | | VTVSS |
| | | | VKKPG | | | | MG | | QTFEG | | AYMELN | | FDS | | |
| | | | SSLKV | | | | | | | | SLRSED | | | | |
| | | | SCKAS | | | | | | | | TAVYYC | | | | |
| | | | G | | | | | | | | | | | | |
| ADI-81763 | 39 | IGHV5- | EVQLV | 5397 | YSFISH | 5687 | WVRQMP | 5843 | IIYPGD | 775 | QVTFSA | 6378 | ARQSSSW | 6685 | WGQGTL |
| | | 51 | QSGAE | | WIA | | GKGLEW | | SDIRYS | | DKSIST | | HDVDY | | VTVSS |
| | | | VKKPG | | | | TG | | PSFQG | | AYLQWS | | | | |
| | | | ESLKI | | | | | | | | SLKASD | | | | |
| | | | SCKGS | | | | | | | | TAMYFC | | | | |
| | | | G | | | | | | | | | | | | |
| ADI-81807 | 82 | IGHV3-53 | EVQLV | 5460 | FNVSRN | 5688 | WVRQAP | 437 | VIYSGG | 6066 | RFTISR | 777 | ARFHDIF | 6686 | WGQGTP |
| | | | ETGGD | | YMN | | GKGLEW | | STFYAD | | DNSKNT | | TGYSDAF | | VTVSS |
| | | | LIQPG | | | | VS | | SIQG | | LYLQMN | | DI | | |
| | | | GSLRL | | | | | | | | SLRAED | | | | |
| | | | SCAAS | | | | | | | | TAVYYC | | | | |
| | | | G | | | | | | | | | | | | |
| ADI-81849 | 27 | IGHV3- | EVQLL | 5373 | FTFSNY | 396 | WVRQAP | 472 | LISYDG | 6067 | RFSISR | 872 | ARPSGRV | 6687 | WGQGTT |
| | | 30 | ESGGG | | AMH | | GKGLEW | | TNKYYA | | DNSKNT | | LHDPWAY | | VTVSS |
| | | | VVQPG | | | | VT | | DSVKG | | LYLQMN | | GMDV | | |
| | | | RSLRL | | | | | | | | SLRADD | | | | |
| | | | SCAAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81550 | 6823 | TGGGTCCTCGGTGAAGGTCTCCTGCATGGCTTCTGGAGGCA | | | | | | 7049 | QVQLVQSGAEVKKP | | 7400 | 8 | | 7 | |
| | | CCTTCAGCAGCTATACTGTCAGCTGGGTGCGACAGGCCCCT | | | | | | | GSSVKVSCMASGGT | | | | | | |
| | | GGACAAGGGCTTGAGTGGATGGGAAGGATCATCCCTATCCT | | | | | | | FSSYTVSWVRQAPG | | | | | | |
| | | TGGTATAGCAAACTACGCACAGAACTTCCAGGGCAGAGTCA | | | | | | | QGLEWMGRIIPILG | | | | | | |
| | | CGATTACCGCGGACAAATCCACGGCCACAGTCTACTTGGAG | | | | | | | IANYAQNFQGRVTI | | | | | | |
| | | GTGAGCAGCCTGAGATCTGAGGACACGGCCGTGTATTACTG | | | | | | | TADKSTATVYLEVS | | | | | | |
| | | TGCGAGAGAGGCGGGTTATTCTGGCTCGGGGAGTTCTTACT | | | | | | | SLRSEDTAVYYCAR | | | | | | |
| | | ACTTTGACTACTGGAGCCAGGGAACCCTGGTCACCGTCTCC | | | | | | | EAGYSGSGSSYYFD | | | | | | |
| | | TCA | | | | | | | YWSQGTLVTVSS | | | | | | |
| ADI-81552 | 6823 | CGGGTCCTCGGTGAAGGTCTCCTGCAAGGCCTCTGGAGGCA | | | | | | 7050 | QVQLVQSGAEVKKP | | 7401 | 8 | | 7 | |
| | | CCTTCAGCAGTAATACTGTCAGCTGGGTGCGACAGGCCCCT | | | | | | | GSSVKVSCKASGGT | | | | | | |
| | | GGACAAGGGCTTGAGTGGATGGGAAGGATCATCCCTATCCT | | | | | | | FSSNTVSWVRQAPG | | | | | | |
| | | TGGTATAGCAGACTACGCACAGAAGTTCCAGGGCAGAGTCA | | | | | | | QGLEWMGRIIPILG | | | | | | |
| | | CGATTACCGCGGACAAATCCACGGCCACAGTCTACATGGAG | | | | | | | IADYAQKFQGRVTI | | | | | | |
| | | GTGAGCAGCCTGAGATCTGAGGACACGGCCATGTACTACTG | | | | | | | TADKSTATVYMEVS | | | | | | |
| | | TGCGAGAGAGGCGGGTTATTCTGGCTCGGGGAGTTCTTACT | | | | | | | SLRSEDTAMYYCAR | | | | | | |
| | | ACTTTGACTACTGGAGCCAGGGAACCCTGGTCACTGTCTCC | | | | | | | EAGYSGSGSSYYFD | | | | | | |
| | | TCA | | | | | | | YWSQGTLVTVSS | | | | | | |
| ADI-81601 | 1288 | TGGGTCCTCGGTGAAGGTCTCCTGCAAGGCTTCTGGAGGCA | | | | | | 7051 | QVQLVQSGAEVKKP | | 7402 | 8 | | 8 | |
| | | CCTTCAGCAGCTATAGTATCTCCTGGGTGCGACAGGCCCCT | | | | | | | GSSVKVSCKASGGT | | | | | | |
| | | GGACAGGGGCCTGAGTGGTTGGGAAAGATCATCCCTATCCT | | | | | | | FSSYSISWVRQAPG | | | | | | |
| | | TGGTGCAACAGACTACGCACAGAAGTTCCAGGGCAGAGTCA | | | | | | | QGPEWLGKIIPILG | | | | | | |
| | | CCATTATCGCGGACAAATCCACGAGCACGGCCTATATGGAG | | | | | | | ATDYAQKFQGRVTI | | | | | | |
| | | CTGAGCAGCCTGAGATCTGAGGACACGGCCGTGTATTATTG | | | | | | | IADKSTSTAYMELS | | | | | | |
| | | TGCGAGAGAGTCGGGGTACTCTGGTTCGGGGAGTGTTACGT | | | | | | | SLRSEDTAVYYCAR | | | | | | |
| | | ACTTCCATCTCTGGGGCCGTGGCACCCTGGTCACCGTCTCC | | | | | | | ESGYSGSGSVTYFH | | | | | | |
| | | TCA | | | | | | | LWGRGTLVTVSS | | | | | | |
| ADI-81653 | 1291 | GAGGTGCAGCTGTTGGAGTCGGGGGGAGGCGTGATCCAGCC | | | | | | 7052 | EVQLLESGGGVIQP | | 7403 | 8 | | 6 | |
| | | TGGGAGGTCCCTGAGACTCTCCTGTGCAGCGTCTGGATTCA | | | | | | | GRSLRLSCAASGFI | | | | | | |
| | | TTTTCAGTGACTATGGCATTCATTGGGTCCGCCAGGCTCCA | | | | | | | FSDYGIHWVRQAPG | | | | | | |
| | | GGCAAGGGGCTGGAGTGGGTGGCAGTTATATGGTATGATGG | | | | | | | KGLEWVAVIWYDGS | | | | | | |
| | | AAGTAATACATACTATGCAGACTCCGTGAAGGGCCGATTCA | | | | | | | NTYYADSVKGRFTI | | | | | | |
| | | CCATCTCCAGAGACAATTCCAAGAACACGGTGTATCTGCAA | | | | | | | SRDNSKNTVYLQMN | | | | | | |
| | | ATGAACAGCCTGAGAGCCGAAGACACGGCTGTGTATTACTG | | | | | | | SLRAEDTAVYYCAR | | | | | | |
| | | TGCGAGACTGGCTACGGTTCCGTACTTCTACTACATGGACG | | | | | | | LATVPYFYYMDVWG | | | | | | |
| | | TCTGGGGCAAAGGGACCACGGTCACCGTCTCCTCA | | | | | | | KGTTVTVSS | | | | | | |
| ADI-81661 | 1286 | TGGGTCCTCGCTGAAGGTCTCCTGCAAGGCTTCTGGAGGCA | | | | | | 7053 | EVQLVESGAEVKKP | | 7404 | 8 | | 7 | |
| | | CCTTCAGCAGCTATACTATCACCTGGGTGCGACAGGCCCCT | | | | | | | GSSLKVSCKASGGT | | | | | | |
| | | GGACAAGGGCTTGAGTGGATGGGCAGGATCATCCCTATTCT | | | | | | | FSSYTITWVRQAPG | | | | | | |
| | | TGGTATACCAAACTACGCACAGACGTTCGAGGGCAGAGTCA | | | | | | | QGLEWMGRIIPILG | | | | | | |
| | | CCATTACCGCGGACAAATCCACGAGCATGGCGTACATGGAA | | | | | | | IPNYAQTFEGRVTI | | | | | | |
| | | CTAAACAGCTTGAGATCTGAGGACACGGCCGTGTATTACTG | | | | | | | TADKSTSMAYMELN | | | | | | |
| | | TGCGACAGAAGTTGGATATAGTGGCTACGGCCCAAGTCCCT | | | | | | | SLRSEDTAVYYCAT | | | | | | |
| | | ACTTTGACTCTTGGGGCCAGGGAACCCTGGTCACCGTCTCC | | | | | | | EVGYSGYGPSPYFD | | | | | | |
| | | TCA | | | | | | | SWGQGTLVTVSS | | | | | | |
| ADI-81763 | 1286 | GAGGTGCAGCTGGTGCAGTCTGGAGCAGAGGTGAAAAAGCC | | | | | | 7054 | EVQLVQSGAEVKKP | | 7405 | 8 | | 7 | |
| | | CGGGGAGTCTCTGAAGATCTCCTGTAAGGGTTCTGGATACA | | | | | | | GESLKISCKGSGYS | | | | | | |
| | | GTTTTATCAGCCATTGGATCGCCTGGGTGCGCCAGATGCCC | | | | | | | FISHWIAWVRQMPG | | | | | | |
| | | GGGAAAGGCCTGGAGTGGACGGGGATCATCTATCCTGGTGA | | | | | | | KGLEWTGIIYPGDS | | | | | | |
| | | CTCTGATATCAGATATAGCCCGTCCTTCCAAGGCCAGGTCA | | | | | | | DIRYSPSFQGQVTF | | | | | | |
| | | CCTTCTCGGCCGACAAGTCCATCAGCACCGCCTACCTGCAG | | | | | | | SADKSISTAYLQWS | | | | | | |
| | | TGGAGCAGCCTGAAGGCCTCGGACACCGCCATGTATTTCTG | | | | | | | SLKASDTAMYFCAR | | | | | | |
| | | TGCGAGGCAAAGCAGCAGCTGGCACGACGTTGACTACTGGG | | | | | | | QSSSWHDVDYWGQG | | | | | | |
| | | GCCAGGGAACCCTGGTCACCGTCTCCTCA | | | | | | | TLVTVSS | | | | | | |
| ADI-81807 | 6805 | GAGGTGCAGCTGGTGGAGACTGGAGGAGACTTGATCCAGCC | | | | | | 7055 | EVQLVETGGDLIQP | | 7406 | 8 | | 7 | |
| | | TGGGGGGTCCCTGAGACTCTCCTGTGCAGCCTCTGGGTTCA | | | | | | | GGSLRLSCAASGFN | | | | | | |
| | | ACGTCAGTAGAAACTACATGAACTGGGTCCGCCAGGCTCCA | | | | | | | VSRNYMNWVRQAPG | | | | | | |
| | | GGGAAGGGGCTGGAGTGGGTCTCAGTTATCTATAGCGGTGG | | | | | | | KGLEWVSVIYSGGS | | | | | | |
| | | TAGTACATTCTACGCAGACTCCATACAGGGCCGATTCACCA | | | | | | | TFYADSIQGRFTIS | | | | | | |
| | | TCTCCAGAGACAATTCCAAGAACACGCTGTATCTTCAAATG | | | | | | | RDNSKNTLYLQMNS | | | | | | |
| | | AACAGCCTGAGAGCCGAGGACACGGCCGTGTATTACTGTGC | | | | | | | LRAEDTAVYYCARF | | | | | | |
| | | GAGATTTCACGATATTTTCACTGGTTACTCCGATGCTTTTG | | | | | | | HDIFTGYSDAFDIW | | | | | | |
| | | ATATCTGGGGCCAAGGGACCCCGGTCACCGTCTCCTCA | | | | | | | GQGTPVTVSS | | | | | | |
| ADI-81849 | 1287 | TGGGAGGTCCCTGAGACTCTCCTGTGCAGCCTCGGGATTCA | | | | | | 7056 | EVQLLESGGGVVQP | | 7407 | 8 | | 6 | |
| | | CCTTCAGTAACTATGCTATGCACTGGGTCCGCCAGGCTCCA | | | | | | | GRSLRLSCAASGFT | | | | | | |
| | | GGCAAGGGGCTGGAGTGGGTGACACTTATATCATATGATGG | | | | | | | FSNYAMHWVRQAPG | | | | | | |
| | | GACCAATAAATATTACGCAGACTCCGTGAAGGGCCGATTCA | | | | | | | KGLEWVTLISYDGT | | | | | | |
| | | GTATCTCCAGAGACAATTCCAAGAACACGCTGTATCTGCAA | | | | | | | NKYYADSVKGRFSI | | | | | | |
| | | ATGAACAGCCTGAGAGCTGACGACACGGCTGTCTATTACTG | | | | | | | SRDNSKNTLYLQMN | | | | | | |
| | | TGCGAGGCCGTCGGGGAGGGTGCTACACGACCCCTGGGCCT | | | | | | | SLRADDTAVYYCAR | | | | | | |
| | | ACGGTATGGACGTCTGGGGCCAAGGGACCACGGTCACCGTC | | | | | | | PSGRVLHDPWAYGM | | | | | | |
| | | TCCTCA | | | | | | | DVWGQGTTVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81851 | 29 | IGHV4- | EVQLL | 5346 | GSISSG | 5689 | WIRQHP | 450 | YISYSG | 6068 | RVIISV | 6379 | ARTYYFD | 6688 | WGQGTL |
| | | 31 | ESGPG | | AYYWS | | GKGLEW | | NIDYNP | | DTSKNQ | | SSGNYYQ | | VTVSS |
| | | | LVKPS | | | | IG | | SLKS | | FSLKLS | | YYFDY | | |
| | | | QTLSL | | | | | | | | SVTAAD | | | | |
| | | | TCTVS | | | | | | | | TAVYYC | | | | |
| | | | G | | | | | | | | | | | | |
| ADI-81747 | 23 | IGHV1- | EVQLL | 5461 | GTFSNS | 5690 | WVRQAP | 433 | GIIPLF | 6069 | RVTITA | 6380 | ARGSLYH | 6689 | WGKGTT |
| | | 69 | ESGSE | | AIN | | GQGLEW | | GTANYA | | DKSTRT | | DTRWGSF | | VTVSS |
| | | | VKKPG | | | | MG | | QKFQD | | AYMELS | | DNHYYMD | | |
| | | | SSVKV | | | | | | | | SLRSED | | V | | |
| | | | SCQAS | | | | | | | | TAVYYC | | | | |
| | | | G | | | | | | | | | | | | |
| ADI-81753 | 29 | IGHV3- | EVQLL | 5355 | FSVRTN | 5691 | WVRQAP | 437 | VIYSDD | 6070 | RFTISR | 6381 | ARVVTEA | 6690 | WGQGTT |
| | | 53 | ESGGG | | YMS | | GKGLEW | | NTNYAD | | HNSKNT | | FDI | | VTVSS |
| | | | LVQPG | | | | VS | | SVKG | | LYLQMN | | | | |
| | | | GSLRL | | | | | | | | SLRAED | | | | |
| | | | SCAAS | | | | | | | | TAVYYC | | | | |
| | | | G | | | | | | | | | | | | |
| ADI-81874 | 51 | IGHV3- | EVQLV | 5462 | FTFSSY | 332 | WVRQAP | 5844 | TIKQDG | 6071 | RFTISR | 6382 | ARVGPYY | 6691 | WGQGTT |
| | | 7 | QSGGG | | WMS | | GKGLEW | | SEKLYV | | DNAKNS | | YYGMDV | | VTVSS |
| | | | LVQPG | | | | AA | | ESVKG | | LYLQMN | | | | |
| | | | GSLRL | | | | | | | | SLGAED | | | | |
| | | | SCAAS | | | | | | | | TAVYYC | | | | |
| | | | R | | | | | | | | | | | | |
| ADI-81879 | 56 | IGHV3- | EVQLL | 5324 | FTFDDH | 5692 | WVRQAP | 437 | GISWNS | 6072 | RFTISR | 6383 | AKSHDIL | 6692 | WGQGTL |
| | | 9 | ESGGG | | AMH | | GKGLEW | | NNIGYG | | DNAKNS | | DGYLDQI | | VTVSS |
| | | | LVQPG | | | | VS | | DSVKG | | LFLQMN | | NYFDY | | |
| | | | RSLRL | | | | | | | | SLRADD | | | | |
| | | | SCAAS | | | | | | | | TALYYC | | | | |
| | | | G | | | | | | | | | | | | |
| ADI-81547 | 14 | IGHV3- | QVQLV | 10 | FTFSSY | 5693 | WVRQAP | 431 | VIWSDG | 6073 | RFTISR | 777 | ARDDGSP | 6693 | WGQGTL |
| | | 33 | QSGGG | | AIH | | GKGLEW | | SHQHYA | | DNSKNT | | TTYFGD | | VTVSS |
| | | | VVQPG | | | | VA | | DSVKG | | LYLQMN | | | | |
| | | | RSLRL | | | | | | | | SLRAED | | | | |
| | | | SCAAS | | | | | | | | TAVYYC | | | | |
| | | | G | | | | | | | | | | | | |
| ADI-81556 | 22 | IGHV1- | QVQLE | 5447 | GTFSSY | 363 | WVRQAP | 433 | RIIPAL | 6074 | RVTITA | 6384 | ARDVGYS | 6694 | WGQGTL |
| | | 69 | QSGAE | | TIS | | GQGLEW | | GIADYA | | DKSTST | | GSGSSYY | | VTVSS |
| | | | VKKPG | | | | MG | | QNFQD | | AYMELS | | FDY | | |
| | | | SSVKV | | | | | | | | SLRSED | | | | |
| | | | SCKAS | | | | | | | | TAVYFC | | | | |
| | | | G | | | | | | | | | | | | |
| ADI-81610 | 67 | IGHV1- | QVQLE | 5447 | GTFSSH | 5694 | WVRQAP | 433 | RIIPIL | 6075 | RVTITA | 6385 | AREIGYS | 6695 | WGQGTL |
| | | 69 | QSGAE | | SIT | | GQGLEW | | DIANYA | | DKSTST | | DYGATEW | | VTVSS |
| | | | VKKPG | | | | MG | | QKFQG | | AYMELS | | FDP | | |
| | | | SSVKV | | | | | | | | SLRYED | | | | |
| | | | SCKAS | | | | | | | | TAVFYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81851 | 1286 | GAGGTGCAGCTGTTGGAGTCGGGCCCAGGACTGGTGAAGCC | | | | | | 7057 | EVQLLESGPGLVKP | | 7408 | 8 | | 7 | |
| | | TTCACAGACCCTGTCCCTCACCTGCACTGTCTCTGGTGGCT | | | | | | | SQTLSLTCTVSGGS | | | | | | |
| | | CCATCAGCAGTGGTGCTTACTACTGGAGCTGGATCCGCCAG | | | | | | | ISSGAYYWSWIRQH | | | | | | |
| | | CACCCAGGGAAGGGCCTGGAGTGGATTGGGTACATCTCTTA | | | | | | | PGKGLEWIGYISYS | | | | | | |
| | | CAGTGGGAATATCGACTACAACCCGTCCCTCAAGAGTCGAG | | | | | | | GNIDYNPSLKSRVI | | | | | | |
| | | TTATCATATCAGTAGACACGTCTAAGAACCAGTTCTCCCTG | | | | | | | ISVDTSKNQFSLKL | | | | | | |
| | | AAGCTGAGCTCTGTGACTGCCGCGGACACGGCCGTGTATTA | | | | | | | SSVTAADTAVYYCA | | | | | | |
| | | CTGTGCGAGAACGTATTACTTTGATAGTAGTGGTAATTATT | | | | | | | RTYYFDSSGNYYQY | | | | | | |
| | | ACCAGTACTACTTTGACTACTGGGGCCAGGGAACCCTGGTC | | | | | | | YFDYWGQGTLVTVS | | | | | | |
| | | ACCGTCTCCTCA | | | | | | | S | | | | | | |
| ADI-81747 | 1291 | GAGGTGCAGCTGTTGGAGTCTGGGTCTGAGGTGAAGAAGCC | | | | | | 7058 | EVQLLESGSEVKKP | | 7409 | 7 | | 7 | |
| | | TGGGTCCTCGGTGAAGGTCTCATGCCAGGCCTCTGGAGGCA | | | | | | | GSSVKVSCQASGGT | | | | | | |
| | | CCTTCAGTAACTCTGCTATCAACTGGGTGCGCCAGGCCCCT | | | | | | | FSNSAINWVRQAPG | | | | | | |
| | | GGACAAGGCCTTGAGTGGATGGGAGGGATCATCCCTCTCTT | | | | | | | QGLEWMGGIIPLFG | | | | | | |
| | | TGGGACAGCAAACTACGCACAGAAGTTCCAGGACAGAGTCA | | | | | | | TANYAQKFQDRVTI | | | | | | |
| | | CGATTACCGCGGACAAATCCACGAGGACAGCCTACATGGAG | | | | | | | TADKSTRTAYMELS | | | | | | |
| | | TTGAGCAGCCTGAGATCTGAGGACACGGCCGTCTATTACTG | | | | | | | SLRSEDTAVYYCAR | | | | | | |
| | | TGCGAGAGGCTCCCTTTATCACGATACCAGATGGGGGAGTT | | | | | | | GSLYHDTRWGSFDN | | | | | | |
| | | TTGACAACCACTACTACATGGACGTCTGGGGCAAAGGGACC | | | | | | | HYYMDVWGKGTTVT | | | | | | |
| | | ACGGTCACCGTCTCCTCA | | | | | | | VSS | | | | | | |
| ADI-81753 | 1287 | GAGGTGCAGCTGTTGGAGTCTGGAGGAGGCTTGGTCCAGCC | | | | | | 7059 | EVQLLESGGGLVQP | | 7410 | 7 | | 7 | |
| | | TGGGGGGTCCCTGAGACTCTCCTGTGCAGCCTCTGGGTTCA | | | | | | | GGSLRLSCAASGFS | | | | | | |
| | | GCGTCAGAACCAACTACATGAGCTGGGTCCGCCAGGCTCCA | | | | | | | VRTNYMSWVRQAPG | | | | | | |
| | | GGGAAGGGGCTGGAGTGGGTCTCAGTTATCTATAGCGATGA | | | | | | | KGLEWVSVIYSDDN | | | | | | |
| | | TAACACAAACTACGCAGACTCCGTGAAGGGCCGATTCACCA | | | | | | | TNYADSVKGRFTIS | | | | | | |
| | | TCTCCAGACACAATTCCAAGAACACGCTGTATCTTCAAATG | | | | | | | RHNSKNTLYLQMNS | | | | | | |
| | | AACAGCCTGAGAGCTGAGGACACGGCCGTGTATTACTGTGC | | | | | | | LRAEDTAVYYCARV | | | | | | |
| | | GAGAGTGGTTACCGAGGCTTTTGATATCTGGGGCCAAGGGA | | | | | | | VTEAFDIWGQGTTV | | | | | | |
| | | CAACGGTCACCGTCTCCTCA | | | | | | | TVSS | | | | | | |
| ADI-81874 | 1287 | GAGGTGCAGCTGGTGCAGTCTGGGGGAGGCTTGGTCCAGCC | | | | | | 7060 | EVQLVQSGGGLVQP | | 7411 | 7 | | 6 | |
| | | TGGGGGGTCCCTGAGACTCTCCTGTGCAGCCTCTAGATTCA | | | | | | | GGSLRLSCAASRFT | | | | | | |
| | | CCTTTAGTAGCTATTGGATGAGCTGGGTCCGCCAGGCTCCA | | | | | | | FSSYWMSWVRQAPG | | | | | | |
| | | GGGAAGGGGCTGGAGTGGGCGGCCACCATAAAGCAAGATGG | | | | | | | KGLEWAATIKQDGS | | | | | | |
| | | AAGTGAGAAATTGTATGTGGAGTCTGTGAAGGGCCGATTCA | | | | | | | EKLYVESVKGRFTI | | | | | | |
| | | CCATCTCCAGAGACAACGCCAAGAACTCACTGTATCTGCAA | | | | | | | SRDNAKNSLYLQMN | | | | | | |
| | | ATGAACAGCCTGGGAGCCGAGGACACGGCTGTTTATTACTG | | | | | | | SLGAEDTAVYYCAR | | | | | | |
| | | TGCGAGAGTGGGCCCTTACTACTACTACGGTATGGACGTCT | | | | | | | VGPYYYYGMDVWGQ | | | | | | |
| | | GGGGCCAAGGGACCACGGTCACCGTCTCCTCA | | | | | | | GTTVTVSS | | | | | | |
| ADI-81879 | 1286 | TGGCAGGTCCCTGAGACTCTCCTGTGCAGCCTCTGGATTCA | | | | | | 7061 | EVQLLESGGGLVQP | | 7412 | 7 | | 6 | |
| | | CGTTTGACGATCATGCCATGCACTGGGTCCGGCAAGCTCCA | | | | | | | GRSLRLSCAASGFT | | | | | | |
| | | GGGAAGGGCCTGGAGTGGGTCTCAGGTATTAGTTGGAATAG | | | | | | | FDDHAMHWVRQAPG | | | | | | |
| | | TAATAACATAGGCTATGGGGACTCTGTGAAGGGCCGATTCA | | | | | | | KGLEWVSGISWNSN | | | | | | |
| | | CCATCTCCAGAGACAACGCCAAGAACTCCCTGTTTCTGCAA | | | | | | | NIGYGDSVKGRFTI | | | | | | |
| | | ATGAACAGTCTGAGAGCTGACGACACGGCCTTGTATTACTG | | | | | | | SRDNAKNSLFLQMN | | | | | | |
| | | TGCAAAGTCCCACGATATTTTGGATGGTTATCTCGACCAAA | | | | | | | SLRADDTALYYCAK | | | | | | |
| | | TAAACTACTTTGACTACTGGGGCCAGGGAACCCTGGTCACC | | | | | | | SHDILDGYLDQINY | | | | | | |
| | | GTCTCCTCA | | | | | | | FDYWGQGTLVTVSS | | | | | | |
| ADI-81547 | 1286 | CAGGTGCAGCTGGTGCAGTCTGGGGGAGGCGTGGTCCAGCC | | | | | | 7062 | QVQLVQSGGGVVQP | | 7413 | 6 | | 6 | |
| | | TGGGAGGTCCCTGAGACTCTCCTGTGCAGCGTCTGGATTCA | | | | | | | GRSLRLSCAASGFT | | | | | | |
| | | CCTTCAGTAGCTACGCCATACACTGGGTCCGCCAGGCTCCA | | | | | | | FSSYAIHWVRQAPG | | | | | | |
| | | GGCAAGGGGCTGGAGTGGGTGGCAGTTATCTGGTCTGATGG | | | | | | | KGLEWVAVIWSDGS | | | | | | |
| | | AAGTCATCAACACTATGCAGACTCCGTGAAGGGGAGATTCA | | | | | | | HQHYADSVKGRFTI | | | | | | |
| | | CCATCTCCAGAGACAATTCCAAGAACACACTGTATCTGCAA | | | | | | | SRDNSKNTLYLQMN | | | | | | |
| | | ATGAACAGCCTGAGAGCCGAGGACACGGCTGTGTATTACTG | | | | | | | SLRAEDTAVYYCAR | | | | | | |
| | | TGCGAGAGATGACGGATCACCTACCACTTACTTTGGCGACT | | | | | | | DDGSPTTYFGDWGQ | | | | | | |
| | | GGGGCCAGGGAACCCTGGTCACCGTCTCCTCA | | | | | | | GTLVTVSS | | | | | | |
| ADI-81556 | 1286 | TGGGTCTTCGGTGAAGGTCTCCTGCAAGGCTTCTGGAGGCA | | | | | | 7063 | QVQLEQSGAEVKKP | | 7414 | 6 | | 5 | |
| | | CCTTCAGCAGCTATACTATCAGCTGGGTGCGACAGGCCCCT | | | | | | | GSSVKVSCKASGGT | | | | | | |
| | | GGACAAGGGCTTGAGTGGATGGGAAGGATCATCCCTGCCCT | | | | | | | FSSYTISWVRQAPG | | | | | | |
| | | TGGTATAGCAGACTACGCACAGAACTTCCAGGACAGAGTCA | | | | | | | QGLEWMGRIIPALG | | | | | | |
| | | CGATTACCGCGGACAAATCCACGAGCACAGCCTACATGGAG | | | | | | | IADYAQNFQDRVTI | | | | | | |
| | | CTGAGCAGCCTGAGATCTGAGGACACGGCCGTATATTTCTG | | | | | | | TADKSTSTAYMELS | | | | | | |
| | | TGCGAGAGATGTGGGTTACTCTGGGTCGGGGAGTTCTTACT | | | | | | | SLRSEDTAVYFCAR | | | | | | |
| | | ACTTTGACTACTGGGGCCAGGGAACCCTGGTCACCGTCTCC | | | | | | | DVGYSGSGSSYYFD | | | | | | |
| | | TCA | | | | | | | YWGQGTLVTVSS | | | | | | |
| ADI-81610 | 1286 | TGGGTCCTCGGTGAAGGTCTCCTGCAAGGCTTCTGGGGGCA | | | | | | 7064 | QVQLEQSGAEVKKP | | 7415 | 6 | | 6 | |
| | | CCTTCAGCAGCCATAGTATTACCTGGGTGCGACAGGCCCCT | | | | | | | GSSVKVSCKASGGT | | | | | | |
| | | GGACAAGGGCTTGAGTGGATGGGAAGGATCATCCCTATCCT | | | | | | | FSSHSITWVRQAPG | | | | | | |
| | | TGATATAGCAAACTACGCACAGAAATTCCAGGGCAGAGTAA | | | | | | | QGLEWMGRIIPILD | | | | | | |
| | | CGATTACCGCGGACAAATCCACGAGCACAGCCTACATGGAA | | | | | | | IANYAQKFQGRVTI | | | | | | |
| | | CTGAGCAGCCTGAGATATGAGGATACGGCCGTGTTTTATTG | | | | | | | TADKSTSTAYMELS | | | | | | |
| | | TGCGAGAGAAATCGGATATAGTGACTACGGGGCAACGGAGT | | | | | | | SLRYEDTAVFYCAR | | | | | | |
| | | GGTTCGACCCCTGGGGCCAGGGAACCCTGGTCACCGTCTCC | | | | | | | EIGYSDYGATEWFD | | | | | | |
| | | TCA | | | | | | | PWGQGTLVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81764 | 40 | IGHV5- | QVQLV | 174 | YSFTSY | 399 | WVRQMP | 476 | LIYPGD | 6076 | QVTMSA | 6386 | ASSASAY | 6696 | WGKGTT |
| | | 51 | QSGAE | | WIA | | GKGLEW | | SDTRYS | | DKSIST | | YLFFYMD | | VTVSS |
| | | | VKKPG | | | | MG | | PSFQG | | AYLQWN | | V | | |
| | | | ESLKI | | | | | | | | SLKASD | | | | |
| | | | SCKGS | | | | | | | | TAVYYC | | | | |
| | | | G | | | | | | | | | | | | |
| ADI-81868 | 46 | IGHV4- | EVQLL | 5346 | GSISSG | 5695 | WIRQHP | 450 | HIYYSG | 6077 | RVTISE | 6387 | ARDRDGD | 6697 | WGQGTL |
| | | 31 | ESGPG | | GYYWG | | GKGLEW | | GTNYNP | | DTSKNQ | | FSFDF | | VTVSS |
| | | | LVKPS | | | | IG | | SLKS | | FSLKLS | | | | |
| | | | QTLSL | | | | | | | | SVTAAD | | | | |
| | | | TCTVS | | | | | | | | TAVYYC | | | | |
| | | | G | | | | | | | | | | | | |
| ADI-81876 | 53 | IGHV3- | EVQLL | 5355 | FTFSSY | 332 | WVRQAP | 5844 | SIKLDG | 6078 | RYTISR | 6388 | ARKRWLQ | 6698 | WGQGTL |
| | | 7 | ESGGG | | WMS | | GKGLEW | | SEKYYV | | DNAKNS | | SDFDY | | VTVSS |
| | | | LVQPG | | | | AA | | VSAKG | | LYLQMN | | | | |
| | | | GSLRL | | | | | | | | SLRAED | | | | |
| | | | SCAAS | | | | | | | | TAVYYC | | | | |
| | | | G | | | | | | | | | | | | |
| ADI-81800 | 76 | IGHV3- | EVQLL | 5324 | FTFDDH | 5692 | WVRQAP | 437 | GISWNS | 6079 | RFTISR | 6383 | AKSHDIL | 6699 | WGQGTL |
| | | 9 | ESGGG | | AMH | | GKGLEW | | GNIGYG | | DNAKNS | | NGYLDQI | | VTVSS |
| | | | LVQPG | | | | VS | | DSVKG | | LFLQMN | | NYFDY | | |
| | | | RSLRL | | | | | | | | SLRADD | | | | |
| | | | SCAAS | | | | | | | | TALYYC | | | | |
| | | | G | | | | | | | | | | | | |
| ADI-81892 | 68 | IGHV3- | EVQLV | 5321 | FIFDDY | 395 | WVRQAP | 437 | GISWNS | 6080 | RFTISR | 6389 | AKDKSSN | 6700 | WGQGTT |
| | | 9 | ESGGG | | AMH | | GKGLEW | | ASLGYA | | DNVKNS | | YGDYAGG | | VTVSS |
| | | | LVQPG | | | | VS | | DSVKG | | LSLQMN | | LGV | | |
| | | | RSLRL | | | | | | | | SLRAED | | | | |
| | | | SCAAS | | | | | | | | TALYYC | | | | |
| | | | G | | | | | | | | | | | | |
| ADI-81657 | 30 | IGHV3- | EVQLV | 5332 | FTFSSY | 5696 | WVRQAP | 486 | RIKSDG | 6081 | RFTFSR | 6390 | ASAPSYY | 6701 | WGKGTT |
| | | 74 | ESGGG | | WMH | | GKGLVW | | SSTTYA | | DNAKNT | | YMDV | | VTVSS |
| | | | LVQPG | | | | VS | | DSVKG | | LYLQMD | | | | |
| | | | GSLRL | | | | | | | | SLRAED | | | | |
| | | | SCAAS | | | | | | | | TAVYYC | | | | |
| | | | G | | | | | | | | | | | | |
| ADI-81776 | 52 | IGHV3- | EVQLL | 5373 | FTFSDY | 5697 | WVRQAP | 431 | VIWYHG | 6082 | RFTISR | 777 | ARDGGFK | 6702 | WGKGTT |
| | | 33 | ESGGG | | GMH | | GKGLEW | | SDKYYA | | DNSKNT | | PIYPRYY | | VTVSS |
| | | | VVQPG | | | | VA | | DSVKG | | LYLQMN | | YYYMDV | | |
| | | | RSLRL | | | | | | | | SLRAED | | | | |
| | | | SCAAS | | | | | | | | TAVYYC | | | | |
| | | | G | | | | | | | | | | | | |
| ADI-81810 | 84 | IGHV3- | EVQLV | 5463 | FTFDDY | 214 | WVRQAP | 437 | GISGNS | 732 | RFTISR | 6391 | AKTMAPD | 6703 | WGQGTT |
| | | 9 | QSGGG | | AMH | | GKGLEW | | GTIGYA | | DNAKNS | | RAHYDTA | | VTVSS |
| | | | LVQPG | | | | VS | | DSVKG | | LYLQMN | | NDAFDI | | |
| | | | RSLRL | | | | | | | | RLRAED | | | | |
| | | | SCAAS | | | | | | | | TALYFC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81764 | 1291 | CAGGTCCAGCTGGTGCAGTCTGGAGCAGAGGTGAAAAAGCC | | | | | | 7065 | QVQLVQSGAEVKKP | | 7416 | 6 | | 5 | |
| | | CGGGGAGTCTCTGAAGATCTCCTGTAAGGGTTCTGGATACA | | | | | | | GESLKISCKGSGYS | | | | | | |
| | | GCTTTACCAGCTACTGGATCGCCTGGGTGCGCCAGATGCCC | | | | | | | FTSYWIAWVRQMPG | | | | | | |
| | | GGGAAAGGCCTGGAGTGGATGGGGCTCATCTATCCTGGTGA | | | | | | | KGLEWMGLIYPGDS | | | | | | |
| | | CTCTGATACCAGATACAGCCCGTCCTTCCAAGGCCAGGTCA | | | | | | | DTRYSPSFQGQVTM | | | | | | |
| | | CCATGTCAGCCGACAAGTCCATCAGCACCGCCTACCTGCAG | | | | | | | SADKSISTAYLQWN | | | | | | |
| | | TGGAACAGCCTGAAGGCCTCGGACACCGCCGTATATTACTG | | | | | | | SLKASDTAVYYCAS | | | | | | |
| | | TGCGAGTTCAGCATCAGCTTATTACCTCTTCTTCTACATGG | | | | | | | SASAYYLFFYMDVW | | | | | | |
| | | ACGTCTGGGGCAAAGGGACCACGGTCACCGTCTCCTCA | | | | | | | GKGTTVTVSS | | | | | | |
| ADI-81868 | 1286 | GAGGTGCAGCTGTTGGAGTCGGGCCCAGGACTGGTGAAGCC | | | | | | 7066 | EVQLLESGPGLVKP | | 7417 | 6 | | 6 | |
| | | TTCACAGACCCTGTCCCTCACCTGCACTGTCTCTGGTGGCT | | | | | | | SQTLSLTCTVSGGS | | | | | | |
| | | CCATCAGCAGTGGTGGTTACTACTGGGGCTGGATCCGCCAG | | | | | | | ISSGGYYWGWIRQH | | | | | | |
| | | CACCCAGGGAAGGGCCTGGAGTGGATTGGGCACATCTATTA | | | | | | | PGKGLEWIGHIYYS | | | | | | |
| | | CAGTGGGGGCACCAACTACAACCCGTCCCTCAAGAGTCGAG | | | | | | | GGTNYNPSLKSRVT | | | | | | |
| | | TTACCATATCAGAAGACACGTCTAAGAACCAGTTCTCCCTG | | | | | | | ISEDTSKNQFSLKL | | | | | | |
| | | AAGCTGAGCTCTGTGACTGCCGCGGACACGGCCGTATATTA | | | | | | | SSVTAADTAVYYCA | | | | | | |
| | | CTGTGCGAGAGATCGGGACGGTGACTTCTCCTTTGACTTCT | | | | | | | RDRDGDFSFDFWGQ | | | | | | |
| | | GGGGCCAGGGAACCCTGGTCACCGTCTCCTCA | | | | | | | GTLVTVSS | | | | | | |
| ADI-81876 | 1286 | GAGGTGCAGCTGTTGGAGTCTGGGGGAGGCTTGGTCCAGCC | | | | | | 7067 | EVQLLESGGGLVQP | | 7418 | 6 | | 6 | |
| | | TGGGGGGTCCCTGAGACTCTCTTGTGCAGCCTCTGGATTCA | | | | | | | GGSLRLSCAASGFT | | | | | | |
| | | CCTTTAGTAGTTATTGGATGAGCTGGGTCCGCCAGGCTCCA | | | | | | | FSSYWMSWVRQAPG | | | | | | |
| | | GGGAAGGGGCTGGAGTGGGCGGCCAGCATAAAGCTAGATGG | | | | | | | KGLEWAASIKLDGS | | | | | | |
| | | AAGTGAGAAATACTATGTGGTCTCTGCGAAGGGCCGATACA | | | | | | | EKYYVVSAKGRYTI | | | | | | |
| | | CCATCTCCAGAGACAACGCCAAGAACTCACTGTATCTGCAA | | | | | | | SRDNAKNSLYLQMN | | | | | | |
| | | ATGAACAGCCTGAGAGCCGAGGACACGGCCGTGTATTACTG | | | | | | | SLRAEDTAVYYCAR | | | | | | |
| | | TGCGAGAAAGAGATGGCTACAATCAGACTTTGACTACTGGG | | | | | | | KRWLQSDFDYWGQG | | | | | | |
| | | GCCAGGGAACCCTGGTCACCGTCTCCTCA | | | | | | | TLVTVSS | | | | | | |
| ADI-81800 | 1286 | TGGCAGGTCCCTGAGACTCTCCTGTGCAGCCTCTGGATTCA | | | | | | 7068 | EVQLLESGGGLVQP | | 7419 | 5 | | 5 | |
| | | CGTTTGACGATCATGCCATGCACTGGGTCCGGCAAGCTCCA | | | | | | | GRSLRLSCAASGFT | | | | | | |
| | | GGGAAGGGCCTGGAGTGGGTCTCAGGTATTAGTTGGAATAG | | | | | | | FDDHAMHWVRQAPG | | | | | | |
| | | TGGTAACATAGGCTATGGGGACTCTGTGAAGGGCCGATTCA | | | | | | | KGLEWVSGISWNSG | | | | | | |
| | | CCATCTCCAGAGACAACGCCAAGAACTCCCTGTTTCTGCAA | | | | | | | NIGYGDSVKGRFTI | | | | | | |
| | | ATGAACAGTCTGAGAGCTGACGACACGGCCTTGTATTACTG | | | | | | | SRDNAKNSLFLQMN | | | | | | |
| | | TGCAAAGTCCCACGATATTTTGAATGGTTATCTCGACCAAA | | | | | | | SLRADDTALYYCAK | | | | | | |
| | | TAAACTACTTTGACTACTGGGGCCAGGGAACCCTGGTCACC | | | | | | | SHDILNGYLDQINY | | | | | | |
| | | GTCTCCTCA | | | | | | | FDYWGQGTLVTVSS | | | | | | |
| ADI-81892 | 1287 | TGGCAGGTCCCTGAGACTCTCATGTGCAGCCTCTGGATTCA | | | | | | 7069 | EVQLVESGGGLVQP | | 7420 | 5 | | 5 | |
| | | TCTTTGATGATTATGCCATGCACTGGGTCCGGCAAGCTCCA | | | | | | | GRSLRLSCAASGFI | | | | | | |
| | | GGGAAGGGCCTGGAGTGGGTCTCAGGTATTAGTTGGAATAG | | | | | | | FDDYAMHWVRQAPG | | | | | | |
| | | TGCTAGTTTAGGCTATGCGGACTCTGTCAAGGGCCGATTCA | | | | | | | KGLEWVSGISWNSA | | | | | | |
| | | CCATCTCCAGAGACAACGTCAAGAACTCCTTATCTCTGCAA | | | | | | | SLGYADSVKGRFTI | | | | | | |
| | | ATGAACAGTCTGAGAGCTGAGGACACGGCCTTGTATTACTG | | | | | | | SRDNVKNSLSLQMN | | | | | | |
| | | TGCAAAAGATAAATCGTCAAACTACGGTGACTACGCTGGGG | | | | | | | SLRAEDTALYYCAK | | | | | | |
| | | GTCTGGGCGTCTGGGGCCAAGGGACCACGGTCACCGTCTCC | | | | | | | DKSSNYGDYAGGLG | | | | | | |
| | | TCA | | | | | | | VWGQGTTVTVSS | | | | | | |
| ADI-81657 | 1291 | GAGGTGCAGCTGGTGGAGTCCGGGGGAGGCTTAGTTCAGCC | | | | | | 7070 | EVQLVESGGGLVQP | | 7421 | 4 | | 4 | |
| | | TGGGGGGTCCCTGAGACTCTCCTGTGCAGCCTCTGGATTCA | | | | | | | GGSLRLSCAASGFT | | | | | | |
| | | CCTTCAGTAGCTACTGGATGCACTGGGTCCGCCAAGCTCCA | | | | | | | FSSYWMHWVRQAPG | | | | | | |
| | | GGGAAGGGGCTGGTGTGGGTCTCACGTATTAAGAGTGATGG | | | | | | | KGLVWVSRIKSDGS | | | | | | |
| | | GAGTAGCACAACCTACGCGGACTCCGTGAAGGGCCGATTCA | | | | | | | STTYADSVKGRFTF | | | | | | |
| | | CCTTCTCCAGAGACAACGCCAAGAACACGCTTTATCTGCAA | | | | | | | SRDNAKNTLYLQMD | | | | | | |
| | | ATGGACAGTCTGAGAGCCGAGGACACGGCTGTATATTACTG | | | | | | | SLRAEDTAVYYCAS | | | | | | |
| | | TGCAAGTGCCCCCTCCTACTACTACATGGACGTCTGGGGCA | | | | | | | APSYYYMDVWGKGT | | | | | | |
| | | AGGGGACCACGGTCACCGTCTCCTCA | | | | | | | TVTVSS | | | | | | |
| ADI-81776 | 1291 | GAGGTGCAGCTGTTGGAGTCTGGGGGAGGCGTGGTCCAGCC | | | | | | 7071 | EVQLLESGGGVVQP | | 7422 | 4 | | 3 | |
| | | TGGGAGGTCCCTGAGACTCTCCTGTGCAGCGTCTGGATTCA | | | | | | | GRSLRLSCAASGFT | | | | | | |
| | | CCTTCAGTGACTATGGCATGCACTGGGTCCGCCAGGCTCCA | | | | | | | FSDYGMHWVRQAPG | | | | | | |
| | | GGCAAGGGGCTGGAGTGGGTGGCAGTTATATGGTATCATGG | | | | | | | KGLEWVAVIWYHGS | | | | | | |
| | | AAGTGATAAATACTATGCAGACTCCGTGAAGGGCCGATTCA | | | | | | | DKYYADSVKGRFTI | | | | | | |
| | | CCATCTCCAGAGACAATTCGAAGAACACGCTGTATCTCCAA | | | | | | | SRDNSKNTLYLQMN | | | | | | |
| | | ATGAACAGCCTGAGAGCCGAGGACACGGCTGTGTATTACTG | | | | | | | SLRAEDTAVYYCAR | | | | | | |
| | | TGCGAGAGATGGGGGCTTTAAACCGATATATCCTCGCTACT | | | | | | | DGGFKPIYPRYYYY | | | | | | |
| | | ACTACTACTACATGGACGTCTGGGGCAAAGGGACCACGGTC | | | | | | | YMDVWGKGTTVTVS | | | | | | |
| | | ACCGTCTCCTCA | | | | | | | S | | | | | | |
| ADI-81810 | 1287 | GAGGTGCAGCTGGTGCAGTCTGGGGGAGGCTTGGTACAGCC | | | | | | 7072 | EVQLVQSGGGLVQP | | 7423 | 4 | | 4 | |
| | | TGGCAGGTCCCTGAGACTCTCCTGTGCAGCCTCTGGATTCA | | | | | | | GRSLRLSCAASGFT | | | | | | |
| | | CCTTTGATGATTATGCCATGCACTGGGTCCGGCAAGCTCCA | | | | | | | FDDYAMHWVRQAPG | | | | | | |
| | | GGGAAGGGCCTGGAGTGGGTCTCAGGTATTAGTGGGAATAG | | | | | | | KGLEWVSGISGNSG | | | | | | |
| | | TGGTACCATAGGCTATGCGGACTCTGTGAAGGGCCGATTCA | | | | | | | TIGYADSVKGRFTI | | | | | | |
| | | CCATCTCCAGAGACAACGCCAAGAACTCCCTCTATCTGCAA | | | | | | | SRDNAKNSLYLQMN | | | | | | |
| | | ATGAACAGACTGAGAGCTGAGGACACGGCCTTGTATTTCTG | | | | | | | RLRAEDTALYFCAK | | | | | | |
| | | TGCAAAAACTATGGCCCCAGACAGGGCCCATTACGATACCG | | | | | | | TMAPDRAHYDTAND | | | | | | |
| | | CGAACGATGCATTTGATATCTGGGGCCAGGGGACCACGGTC | | | | | | | AFDIWGQGTTVTVS | | | | | | |
| | | ACCGTCTCCTCA | | | | | | | S | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81634 | 7 | IGHV2- | QITPK | 5464 | FSLSTS | 219 | WIRQPP | 441 | LIYWND | 6083 | RLTITK | 6392 | AHRRGDI | 6704 | WGQGTL |
| | | 5 | ESGLT | | GVGVG | | GKALEW | | DKRYSP | | DTSKNQ | | VWPAAI | | VTVSS |
| | | | LVKPT | | | | LA | | SLKN | | VVFTMT | | IDNWFDP | | |
| | | | QTLTL | | | | | | | | NMDPVD | | | | |
| | | | TCTFS | | | | | | | | TATYYC | | | | |
| | | | G | | | | | | | | | | | | |
| ADI-81817 | 91 | IGHV3- | EVQLV | 5321 | FTFNDY | 5698 | WVRQAP | 437 | GISWNS | 6084 | RFTISR | 6393 | VKDKSSN | 6705 | WAQGTT |
| | | 9 | ESGGG | | AMH | | GKGLEW | | GSIGYA | | DNAKNS | | YGDYAGG | | VTVSS |
| | | | LVQPG | | | | VS | | DSVKG | | LYLQMN | | MDV | | |
| | | | RSLRL | | | | | | | | SLRAED | | | | |
| | | | SCAAS | | | | | | | | TAFYYC | | | | |
| | | | G | | | | | | | | | | | | |
| ADI-81805 | 81 | IGHV5- | EVQLL | 5430 | YSFFHY | 422 | WVRQMP | 516 | TINPVN | 764 | QVTISV | 1008 | ATADRG | 1269 | WGQGTL |
| | | 10-1 | ESGAE | | WIS | | GKGLEW | | SYTNYN | | DRSINT | | | | VTVSS |
| | | | VKKPG | | | | MA | | PSFQG | | VYLQWS | | | | |
| | | | ESLRI | | | | | | | | SLRASD | | | | |
| | | | SCKGS | | | | | | | | SAMYYC | | | | |
| | | | G | | | | | | | | | | | | |
| ADI-81576 | 38 | IGHV1- | QVQLV | 5465 | FTFAVS | 5699 | WVRQAR | 5789 | WMVVGS | 6085 | RVTISG | 6394 | AAPSCSR | 6706 | WGQGTT |
| | | 58 | QSGPE | | SVQ | | GQRLEW | | GDTNYA | | DMSTGT | | TTCYDGF | | VTVSS |
| | | | VKMPG | | | | IG | | EKFQE | | VYMEVS | | NL | | |
| | | | TSVTV | | | | | | | | GLKSDD | | | | |
| | | | SCKAS | | | | | | | | TAVYYC | | | | |
| | | | G | | | | | | | | | | | | |
| ADI-81665 | 37 | IGHV3- | EVQLL | 5466 | FTFSRY | 5700 | WVRQAP | 437 | GISYYR | 6086 | RFTISR | 6395 | AKVAGTY | 6707 | WGQGTA |
| | | 30 | ESGGG | | SMH | | GKGLEW | | SDIDYA | | DSSKNT | | CSGTTCS | | VTVSS |
| | | | LVQPG | | | | VS | | DSVNG | | LYLQMN | | SGYFDY | | |
| | | | GSMRI | | | | | | | | SLKDED | | | | |
| | | | SCAAS | | | | | | | | TAVYYC | | | | |
| | | | G | | | | | | | | | | | | |
| ADI-81714 | 84 | IGHV3- | EVQLL | 5467 | FAFHDY | 5701 | WVRQAP | 437 | LIGPDG | 6087 | RFTVSR | 6396 | AKDTSGA | 6708 | WGQGTL |
| | | 43 | ESGGA | | SMH | | GKGLEW | | GGAFYS | | DNSKNS | | KCRVPIF | | VTVSS |
| | | | VVQPG | | | | VS | | DYVKG | | LFLQLN | | EN | | |
| | | | GSLRL | | | | | | | | SMRSED | | | | |
| | | | SCAAS | | | | | | | | TAFYYC | | | | |
| | | | G | | | | | | | | | | | | |
| ADI-81683 | 53 | IGHV1- | QVQLV | 5468 | YTFSDH | 5702 | WVRQAP | 433 | VTNPSA | 6088 | RVTMTT | 6397 | ARDIFFL | 6709 | WGQGTT |
| | | 46 | QSGTE | | YMH | | GQGLEW | | GSTTYA | | DTSTST | | PAGDAFD | | VTVSS |
| | | | VKKPG | | | | MG | | QKFQG | | VYLGLS | | IL | | |
| | | | ASVKV | | | | | | | | NLRPED | | | | |
| | | | SCKAS | | | | | | | | TAVYFC | | | | |
| | | | G | | | | | | | | | | | | |
| ADI-81820 | 1 | IGHV1- | EVQLV | 5469 | YNFVNY | 5703 | WVRQAP | 433 | WINAEY | 6089 | RVTMTT | 6398 | VRVSSSA | 6710 | GGRGTL |
| | | 18 | QSETE | | GIS | | GQGLEW | | GNRKYA | | DTSTST | | TGYFDL | | VTVSS |
| | | | VKKPG | | | | MG | | QMVQG | | AYMELR | | | | |
| | | | ASVKV | | | | | | | | SIRFDD | | | | |
| | | | SCKAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81634 | 1286 | CAGATCACCCCGAAGGAGTCTGGTCTTACGCTGGTGAAACC | | | | | | 7073 | QITPKESGLTLVKP | | 7424 | 2 | | 2 | |
| | | CACACAGACCCTCACGCTGACCTGCACCTTCTCTGGGTTCT | | | | | | | TQTLTLTCTFSGFS | | | | | | |
| | | CACTCAGCACTAGTGGAGTGGGTGTGGGCTGGATCCGTCAG | | | | | | | LSTSGVGVGWIRQP | | | | | | |
| | | CCCCCAGGAAAGGCCCTGGAGTGGCTTGCACTCATTTATTG | | | | | | | PGKALEWLALIYWN | | | | | | |
| | | GAATGATGATAAGCGTTACAGCCCATCTCTGAAGAACAGGC | | | | | | | DDKRYSPSLKNRLT | | | | | | |
| | | TCACCATCACCAAGGACACCTCCAAAAACCAGGTGGTCTTT | | | | | | | ITKDTSKNQVVFTM | | | | | | |
| | | ACAATGACCAACATGGACCCTGTGGACACAGCCACATATTA | | | | | | | TNMDPVDTATYYCA | | | | | | |
| | | CTGTGCACACAGACGGGGGGATATTGTAGTAGTACCAGCTG | | | | | | | HRRGDIVVVPAAII | | | | | | |
| | | CTATTATAGACAACTGGTTCGACCCCTGGGGCCAGGGAACC | | | | | | | DNWFDPWGQGTLVT | | | | | | |
| | | CTGGTCACCGTCTCCTCA | | | | | | | VSS | | | | | | |
| ADI-81817 | 6824 | TGGCAGGTCCCTGAGACTCTCCTGTGCAGCCTCTGGATTCA | | | | | | 7074 | EVQLVESGGGLVQP | | 7425 | 2 | | 2 | |
| | | CCTTTAATGATTATGCCATGCACTGGGTCCGGCAAGCTCCA | | | | | | | GRSLRLSCAASGFT | | | | | | |
| | | GGGAAGGGCCTGGAGTGGGTCTCAGGTATTAGTTGGAATAG | | | | | | | FNDYAMHWVRQAPG | | | | | | |
| | | TGGTAGCATAGGCTATGCGGACTCTGTGAAGGGCCGATTCA | | | | | | | KGLEWVSGISWNSG | | | | | | |
| | | CCATCTCCAGAGACAACGCCAAGAACTCCCTCTATCTGCAA | | | | | | | SIGYADSVKGRFTI | | | | | | |
| | | ATGAACAGTCTGAGAGCTGAGGACACGGCCTTCTATTACTG | | | | | | | SRDNAKNSLYLQMN | | | | | | |
| | | TGTAAAAGATAAATCGTCAAACTACGGTGACTACGCTGGGG | | | | | | | SLRAEDTAFYYCVK | | | | | | |
| | | GTATGGACGTCTGGGCCCAAGGGACCACGGTCACCGTCTCC | | | | | | | DKSSNYGDYAGGMD | | | | | | |
| | | TCA | | | | | | | VWAQGTTVTVSS | | | | | | |
| ADI-81805 | 1286 | GAGGTGCAGCTGTTGGAGTCCGGAGCAGAGGTGAAGAAACC | | | | | | 7075 | EVQLLESGAEVKKP | | 7426 | 20 | | 15 | |
| | | CGGGGAGTCTCTGAGGATCTCCTGTAAGGGTTCTGGATACA | | | | | | | GESLRISCKGSGYS | | | | | | |
| | | GCTTTTTCCATTACTGGATCAGCTGGGTGCGCCAGATGCCC | | | | | | | FFHYWISWVRQMPG | | | | | | |
| | | GGGAAAGGCCTGGAATGGATGGCGACCATTAATCCTGTTAA | | | | | | | KGLEWMATINPVNS | | | | | | |
| | | CTCTTATACCAACTACAACCCGTCCTTCCAAGGCCAAGTCA | | | | | | | YTNYNPSFQGQVTI | | | | | | |
| | | CCATCTCAGTTGACAGGTCCATCAACACTGTCTACCTGCAA | | | | | | | SVDRSINTVYLQWS | | | | | | |
| | | TGGAGCAGTCTGAGGGCCTCGGACAGCGCCATGTATTACTG | | | | | | | SLRASDSAMYYCAT | | | | | | |
| | | TGCGACCGCGGATAGGGGTTGGGGCCAGGGAACCCTGGTCA | | | | | | | ADRGWGQGTLVTVS | | | | | | |
| | | CCGTCTCCTCA | | | | | | | S | | | | | | |
| ADI-81576 | 1287 | CAGGTCCAGCTTGTGCAGTCTGGGCCTGAGGTGAAGATGCC | | | | | | 7076 | QVQLVQSGPEVKMP | | 7427 | 18 | | 16 | |
| | | TGGGACCTCAGTGACGGTCTCCTGTAAGGCTTCTGGATTCA | | | | | | | GTSVTVSCKASGFT | | | | | | |
| | | CCTTTGCTGTCTCTTCTGTGCAGTGGGTGCGACAGGCTCGT | | | | | | | FAVSSVQWVRQARG | | | | | | |
| | | GGACAACGCCTGGAGTGGATAGGCTGGATGGTCGTTGGCAG | | | | | | | QRLEWIGWMVVGSG | | | | | | |
| | | TGGCGACACCAACTACGCAGAGAAGTTCCAGGAAAGAGTCA | | | | | | | DTNYAEKFQERVTI | | | | | | |
| | | CCATTTCCGGGGACATGTCCACAGGCACAGTCTACATGGAA | | | | | | | SGDMSTGTVYMEVS | | | | | | |
| | | GTGAGCGGCCTGAAGTCCGACGACACGGCCGTGTATTACTG | | | | | | | GLKSDDTAVYYCAA | | | | | | |
| | | TGCGGCACCCTCCTGCAGTAGAACCACCTGTTACGATGGTT | | | | | | | PSCSRTTCYDGFNL | | | | | | |
| | | TTAATCTCTGGGGCCAAGGGACCACGGTCACCGTCTCCTCA | | | | | | | WGQGTTVTVSS | | | | | | |
| ADI-81665 | 6825 | GAGGTGCAGCTGTTGGAGTCTGGGGGAGGCTTGGTCCAGCC | | | | | | 7077 | EVQLLESGGGLVQP | | 7428 | 18 | | 17 | |
| | | TGGGGGGTCCATGAGAATATCCTGTGCAGCCTCAGGATTCA | | | | | | | GGSMRISCAASGFT | | | | | | |
| | | CCTTCAGTAGATATAGCATGCACTGGGTCCGCCAGGCTCCA | | | | | | | FSRYSMHWVRQAPG | | | | | | |
| | | GGCAAGGGGCTGGAGTGGGTGTCAGGTATATCATATTATAG | | | | | | | KGLEWVSGISYYRS | | | | | | |
| | | AAGTGATATAGACTATGCAGACTCTGTGAACGGCCGATTCA | | | | | | | DIDYADSVNGRFTI | | | | | | |
| | | CCATCTCCAGAGACAGTTCCAAGAACACGCTGTATCTGCAA | | | | | | | SRDSSKNTLYLQMN | | | | | | |
| | | ATGAACAGCCTGAAAGATGAGGACACGGCTGTGTATTACTG | | | | | | | SLKDEDTAVYYCAK | | | | | | |
| | | TGCGAAAGTTGCCGGGACGTATTGTAGTGGTACTACCTGCT | | | | | | | VAGTYCSGTTCSSG | | | | | | |
| | | CCTCGGGGTACTTTGACTACTGGGGCCAGGGAACCGCGGTC | | | | | | | YFDYWGQGTAVTVS | | | | | | |
| | | ACCGTATCATCA | | | | | | | S | | | | | | |
| ADI-81714 | 1286 | GAGGTGCAGCTGTTGGAGTCTGGGGGAGCCGTGGTACAGCC | | | | | | 7078 | EVQLLESGGAVVQP | | 7429 | 18 | | 17 | |
| | | TGGGGGGTCACTGAGACTCTCCTGTGCAGCCTCTGGATTCG | | | | | | | GGSLRLSCAASGFA | | | | | | |
| | | CCTTTCATGATTATTCCATGCACTGGGTCCGTCAGGCTCCA | | | | | | | FHDYSMHWVRQAPG | | | | | | |
| | | GGGAAGGGTCTGGAGTGGGTCTCTCTTATTGGTCCGGATGG | | | | | | | KGLEWVSLIGPDGG | | | | | | |
| | | TGGTGGCGCATTCTATTCAGACTATGTGAAGGGCCGATTCA | | | | | | | GAFYSDYVKGRFTV | | | | | | |
| | | CCGTCTCCAGAGACAACAGCAAAAACTCCCTCTTTCTACAA | | | | | | | SRDNSKNSLFLQLN | | | | | | |
| | | CTGAACAGTATGAGAAGTGAGGACACCGCCTTCTATTACTG | | | | | | | SMRSEDTAFYYCAK | | | | | | |
| | | TGCAAAAGATACTTCCGGGGCCAAATGTAGGGTACCAATCT | | | | | | | DTSGAKCRVPIFEN | | | | | | |
| | | TTGAGAACTGGGGCCAGGGAACCCTGGTCACCGTCTCCTCA | | | | | | | WGQGTLVTVSS | | | | | | |
| ADI-81683 | 1287 | CAGGTGCAGCTGGTGCAGTCTGGGACTGAGGTGAAGAAGCC | | | | | | 7079 | QVQLVQSGTEVKKP | | 7430 | 16 | | 13 | |
| | | TGGGGCCTCAGTGAAGGTTTCCTGCAAGGCATCGGGATACA | | | | | | | GASVKVSCKASGYT | | | | | | |
| | | CCTTCAGCGACCACTATATGCACTGGGTGCGACAGGCCCCT | | | | | | | FSDHYMHWVRQAPG | | | | | | |
| | | GGACAAGGGCTTGAGTGGATGGGAGTAACCAACCCTAGTGC | | | | | | | QGLEWMGVTNPSAG | | | | | | |
| | | TGGGAGCACAACCTACGCACAGAAGTTCCAGGGCAGAGTCA | | | | | | | STTYAQKFQGRVTM | | | | | | |
| | | CCATGACCACTGACACGTCCACGAGCACGGTCTACCTGGGA | | | | | | | TTDTSTSTVYLGLS | | | | | | |
| | | CTGAGCAACCTGAGACCTGAAGACACGGCCGTTTATTTCTG | | | | | | | NLRPEDTAVYFCAR | | | | | | |
| | | TGCAAGAGATATTTTTTTCTTACCAGCCGGTGATGCTTTTG | | | | | | | DIFFLPAGDAFDLW | | | | | | |
| | | ATCTCTGGGGCCAAGGGACCACGGTCACCGTCTCCTCA | | | | | | | GQGTTVTVSS | | | | | | |
| ADI-81820 | 6826 | GAGGTCCAGCTGGTGCAGTCTGAAACTGAGGTGAAGAAGCC | | | | | | 7080 | EVQLVQSETEVKKP | | 7431 | 16 | | 12 | |
| | | TGGGGCCTCAGTGAAGGTCTCCTGCAAGGCTTCTGGGTATA | | | | | | | GASVKVSCKASGYN | | | | | | |
| | | ACTTTGTCAATTATGGTATAAGTTGGGTGCGACAGGCCCCT | | | | | | | FVNYGISWVRQAPG | | | | | | |
| | | GGACAAGGGCTTGAGTGGATGGGATGGATCAACGCTGAGTA | | | | | | | QGLEWMGWINAEYG | | | | | | |
| | | TGGCAACAGAAAGTATGCACAGATGGTCCAGGGCAGAGTCA | | | | | | | NRKYAQMVQGRVTM | | | | | | |
| | | CCATGACCACAGACACATCCACGAGCACAGCCTACATGGAA | | | | | | | TTDTSTSTAYMELR | | | | | | |
| | | CTGAGGAGTATCAGATTTGACGACACGGCCGTGTATTATTG | | | | | | | SIRFDDTAVYYCVR | | | | | | |
| | | TGTGAGAGTGAGCAGCAGCGCAACCGGGTACTTCGATCTAG | | | | | | | VSSSATGYFDLGGR | | | | | | |
| | | GGGGCCGTGGCACCCTGGTCACCGTCTCCTCA | | | | | | | GTLVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81569 | 32 | IGHV4- | QVQLQ | 56 | GSISSV | 5704 | WIRQHP | 5845 | HIYHNG | 6090 | RVTMSL | 6399 | ARLGPSF | 6588 | WDQGTT |
| | | 31 | QSGPG | | YYYWT | | GKGLEW | | TTYSNP | | DTSKNQ | | YDRSGSS | | VTVSS |
| | | | LVKPS | | | | MG | | SLKS | | FSLKVS | | RLNYFDS | | |
| | | | QTLSL | | | | | | | | SVTAAD | | | | |
| | | | TCTVS | | | | | | | | TAVYYC | | | | |
| | | | G | | | | | | | | | | | | |
| ADI-81873 | 50 | IGHV3- | EVQLL | 5470 | FTFSSY | 207 | WVRQTP | 5846 | GIGTTG | 6091 | RFTISR | 6251 | ARGDQWE | 6711 | WGQGTT |
| | | 13 | ESGGG | | DMH | | SNGLEW | | DTYYLD | | ENAKSS | | LLNRYYY | | VTVSS |
| | | | LIQPG | | | | VS | | SVKG | | LYLQMN | | AMDV | | |
| | | | GSLRL | | | | | | | | SLRAGD | | | | |
| | | | SCVAS | | | | | | | | TAVYYC | | | | |
| | | | G | | | | | | | | | | | | |
| ADI-81534 | 3 | IGHV3- | QVQLQ | 154 | FTLSSV | 5705 | WVRQVS | 5847 | GIGSAG | 6092 | RFTISR | 6400 | ARGDHIL | 6712 | WGKGTT |
| | | 13 | ESGGG | | DMH | | GKGLEW | | DTYYAD | | ENARNS | | TGLNYYY | | VTVSS |
| | | | LVQPG | | | | VS | | SVKG | | LYLQMS | | YMDV | | |
| | | | GSLRL | | | | | | | | SLRAGD | | | | |
| | | | SCAAS | | | | | | | | TAVYFC | | | | |
| | | | G | | | | | | | | | | | | |
| ADI-81671 | 42 | IGHV4- | QAQLL | 5471 | GSISSD | 315 | WIRQPP | 445 | TTYYTG | 6093 | RVTISV | 6401 | ARVEVSG | 1148 | WGQGAL |
| | | 39 | ESGPG | | HFYWG | | GKGLEW | | STNYNP | | DTSKNQ | | GFSGYDF | | VTVSS |
| | | | LVKPS | | | | IG | | SLKS | | FSLRLT | | LTPYYFD | | |
| | | | ETLSL | | | | | | | | SVTATD | | Y | | |
| | | | TCTVS | | | | | | | | TAVYYC | | | | |
| | | | G | | | | | | | | | | | | |
| ADI-81749 | 25 | IGHV1- | QVQLV | 5472 | YTFTSQ | 5706 | WVRQAP | 433 | IINP- | 6094 | RVTMTS | 6402 | ARDLVFI | 6713 | WGQGTT |
| | | 46 | ESGAE | | YVH | | GQGLEW | | TGSTGY | | DTSTST | | PAIDALE | | VTVSS |
| | | | VKKPG | | | | MG | | AQKFQG | | IYMELS | | I | | |
| | | | ASVRV | | | | | | | | GLRSED | | | | |
| | | | SCKAS | | | | | | | | TAVYYC | | | | |
| | | | G | | | | | | | | | | | | |
| ADI-81668 | 39 | IGHV4- | EVQLL | 5380 | DSISGY | 5707 | WIRQPP | 445 | YIYNSE | 6095 | RVTMSG | 6403 | ARGMSGG | 6714 | WGQGTT |
| | | 59 | ESGPG | | YWS | | GKGLEW | | TTDYNP | | DTSKNQ | | DAFDI | | VTVSS |
| | | | LVKPS | | | | IG | | SLKS | | VSLRLT | | | | |
| | | | ETLSL | | | | | | | | SVTAAD | | | | |
| | | | TCSVS | | | | | | | | TAVYYC | | | | |
| | | | G | | | | | | | | | | | | |
| ADI-81771 | 47 | IGHV1- | EVQLV | 5402 | DTFTRH | 5708 | WVRQAP | 433 | RIIPLP | 6096 | RVTITA | 6404 | ARHNSPV | 6715 | WGQGTL |
| | | 69 | ESGAE | | SIS | | GQGLEW | | DIPNYA | | DKSTNT | | WGHYDNS | | VTVSS |
| | | | VKKPG | | | | MG | | QKFQG | | AYMELS | | GPFDY | | |
| | | | SSVKV | | | | | | | | TLRSDD | | | | |
| | | | SCKAS | | | | | | | | TAVYYC | | | | |
| | | | G | | | | | | | | | | | | |
| ADI-81865 | 43 | IGHV1- | EVQLV | 5385 | HTFANY | 5709 | WVRQAP | 433 | IPNPSG | 6097 | RVTMTG | 6405 | ARGGIVP | 1130 | WGQGTL |
| | | 46 | ESGAE | | YIH | | GQGLEW | | GSTAYA | | DTSTST | | DAKQYFD | | VTVSS |
| | | | VKKPG | | | | MG | | HNFQD | | VYMELS | | Y | | |
| | | | ASVKV | | | | | | | | SLRSED | | | | |
| | | | SCKAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81569 | 6827 | CAGGTACAGCTGCAGCAGTCGGGCCCAGGACTGGTGAAGCC | | | | | | 7081 | QVQLQQSGPGLVKP | | 7432 | 15 | | 13 | |
| | | TTCACAGACCCTGTCCCTCACCTGCACTGTCTCTGGTGGCT | | | | | | | SQTLSLTCTVSGGS | | | | | | |
| | | CCATCAGCAGTGTTTATTACTACTGGACCTGGATCCGCCAG | | | | | | | ISSVYYYWTWIRQH | | | | | | |
| | | CACCCAGGGAAGGGCCTAGAGTGGATGGGGCACATCTATCA | | | | | | | PGKGLEWMGHIYHN | | | | | | |
| | | CAATGGGACCACCTACTCCAATCCGTCCCTCAAGAGCCGAG | | | | | | | GTTYSNPSLKSRVT | | | | | | |
| | | TTACCATGTCACTGGACACGTCTAAGAACCAGTTTTCCCTG | | | | | | | MSLDTSKNQFSLKV | | | | | | |
| | | AAGGTGAGCTCTGTGACTGCCGCGGACACGGCCGTGTATTA | | | | | | | SSVTAADTAVYYCA | | | | | | |
| | | CTGTGCGAGACTTGGACCGTCTTTCTACGATAGAAGTGGTT | | | | | | | RLGPSFYDRSGSSR | | | | | | |
| | | CCTCACGTCTTAACTACTTTGACTCCTGGGACCAGGGGACC | | | | | | | LNYFDSWDQGTTVT | | | | | | |
| | | ACGGTCACCGTCTCCTCA | | | | | | | VSS | | | | | | |
| ADI-81873 | 1287 | TGGGGGGTCCCTGAGACTCTCCTGTGTAGCCTCTGGATTTA | | | | | | 7082 | EVQLLESGGGLIQP | | 7433 | 15 | | 11 | |
| | | CCTTCAGTAGCTACGACATGCACTGGGTCCGCCAAACTCCG | | | | | | | GGSLRLSCVASGFT | | | | | | |
| | | AGTAACGGTCTGGAGTGGGTCTCAGGTATTGGTACTACTGG | | | | | | | FSSYDMHWVRQTPS | | | | | | |
| | | TGACACATACTATTTGGACTCCGTGAAGGGCCGATTCACCA | | | | | | | NGLEWVSGIGTTGD | | | | | | |
| | | TCTCCAGAGAAAATGCCAAGAGCTCCTTGTATCTTCAAATG | | | | | | | TYYLDSVKGRFTIS | | | | | | |
| | | AACAGCCTGAGAGCCGGGGACACGGCTGTTTATTACTGTGC | | | | | | | RENAKSSLYLQMNS | | | | | | |
| | | AAGAGGAGATCAGTGGGAGCTACTCAATCGCTACTACTACG | | | | | | | LRAGDTAVYYCARG | | | | | | |
| | | CTATGGACGTCTGGGGCCAAGGGACCACGGTCACCGTCTCC | | | | | | | DQWELLNRYYYAMD | | | | | | |
| | | TCA | | | | | | | VWGQGTTVTVSS | | | | | | |
| ADI-81534 | 1291 | TGGGGGGTCCCTGAGACTCTCCTGTGCAGCCTCTGGATTCA | | | | | | 7083 | QVQLQESGGGLVQP | | 7434 | 14 | | 11 | |
| | | CGTTGAGTAGTGTCGACATGCACTGGGTCCGGCAAGTCTCT | | | | | | | GGSLRLSCAASGFT | | | | | | |
| | | GGAAAGGGTCTGGAATGGGTCTCAGGTATTGGTTCTGCTGG | | | | | | | LSSVDMHWVRQVSG | | | | | | |
| | | TGACACATACTATGCAGACTCCGTGAAGGGCCGATTCACCA | | | | | | | KGLEWVSGIGSAGD | | | | | | |
| | | TCTCCAGAGAAAATGCCAGGAACTCCTTGTATCTTCAAATG | | | | | | | TYYADSVKGRFTIS | | | | | | |
| | | AGCAGCCTGAGAGCCGGGGACACGGCTGTCTACTTCTGTGC | | | | | | | RENARNSLYLQMSS | | | | | | |
| | | AAGAGGTGACCATATTTTGACTGGTCTCAATTACTACTACT | | | | | | | LRAGDTAVYFCARG | | | | | | |
| | | ACATGGACGTCTGGGGCAAAGGGACCACGGTCACCGTCTCC | | | | | | | DHILTGLNYYYYMD | | | | | | |
| | | TCA | | | | | | | VWGKGTTVTVSS | | | | | | |
| ADI-81671 | 1285 | CAGGCGCAGCTGTTGGAGTCGGGCCCAGGACTGGTGAAGCC | | | | | | 7084 | QAQLLESGPGLVKP | | 7435 | 13 | | 10 | |
| | | TTCGGAGACCCTGTCCCTCACATGCACTGTCTCTGGCGGCT | | | | | | | SETLSLTCTVSGGS | | | | | | |
| | | CCATCAGCAGTGATCATTTCTACTGGGGCTGGATCCGCCAG | | | | | | | ISSDHFYWGWIRQP | | | | | | |
| | | CCCCCAGGGAAGGGGCTGGAGTGGATTGGGACCACCTATTA | | | | | | | PGKGLEWIGTTYYT | | | | | | |
| | | TACTGGGAGCACCAACTACAACCCGTCCCTCAAGAGTCGGG | | | | | | | GSTNYNPSLKSRVT | | | | | | |
| | | TCACCATATCCGTTGACACGTCCAAGAACCAGTTCTCCCTG | | | | | | | ISVDTSKNQFSLRL | | | | | | |
| | | AGGCTGACCTCTGTGACCGCCACAGACACGGCTGTGTATTA | | | | | | | TSVTATDTAVYYCA | | | | | | |
| | | CTGTGCGCGAGTCGAGGTTTCGGGTGGATTTAGTGGCTACG | | | | | | | RVEVSGGFSGYDFL | | | | | | |
| | | ATTTCCTCACACCGTATTACTTTGACTACTGGGGCCAGGGA | | | | | | | TPYYFDYWGQGALV | | | | | | |
| | | GCCCTGGTCACCGTCTCCTCA | | | | | | | TVSS | | | | | | |
| ADI-81749 | 1287 | CAGGTGCAGCTGGTGGAGTCTGGGGCTGAGGTGAAGAAGCC | | | | | | 7085 | QVQLVESGAEVKKP | | 7436 | 13 | | 9 | |
| | | TGGGGCCTCAGTGAGGGTTTCCTGCAAGGCATCTGGATACA | | | | | | | GASVRVSCKASGYT | | | | | | |
| | | CCTTCACCAGTCAATATGTGCACTGGGTGCGACAGGCCCCT | | | | | | | FTSQYVHWVRQAPG | | | | | | |
| | | GGACAAGGGCTTGAGTGGATGGGAATAATCAACCCTACTGG | | | | | | | QGLEWMGIINPTGS | | | | | | |
| | | AAGTACAGGCTACGCACAGAAGTTCCAGGGCAGAGTCACCA | | | | | | | TGYAQKFQGRVTMT | | | | | | |
| | | TGACCAGTGACACGTCCACGAGCACAATCTACATGGAGCTG | | | | | | | SDTSTSTIYMELSG | | | | | | |
| | | AGTGGCCTGAGATCTGAGGACACGGCCGTCTATTATTGTGC | | | | | | | LRSEDTAVYYCARD | | | | | | |
| | | GAGAGATCTTGTTTTTATACCAGCTATTGATGCTTTAGAAA | | | | | | | LVFIPAIDALEIWG | | | | | | |
| | | TCTGGGGCCAAGGGACCACGGTCACCGTCTCCTCA | | | | | | | QGTTVTVSS | | | | | | |
| ADI-81668 | 1287 | GAGGTGCAGCTGTTGGAGTCGGGCCCAGGACTGGTGAAGCC | | | | | | 7086 | EVQLLESGPGLVKP | | 7437 | 12 | | 12 | |
| | | TTCGGAGACCCTGTCCCTCACCTGCAGTGTCTCTGGTGACT | | | | | | | SETLSLTCSVSGDS | | | | | | |
| | | CCATTAGTGGTTACTACTGGAGCTGGATCCGGCAGCCCCCA | | | | | | | ISGYYWSWIRQPPG | | | | | | |
| | | GGGAAGGGACTGGAGTGGATTGGGTATATCTATAACAGTGA | | | | | | | KGLEWIGYIYNSET | | | | | | |
| | | GACCACCGACTACAACCCCTCCCTCAAGAGTCGAGTCACCA | | | | | | | TDYNPSLKSRVTMS | | | | | | |
| | | TGTCTGGAGACACGTCCAAGAACCAGGTCTCCCTGAGGCTG | | | | | | | GDTSKNQVSLRLTS | | | | | | |
| | | ACCTCTGTGACCGCTGCGGACACGGCCGTATATTACTGTGC | | | | | | | VTAADTAVYYCARG | | | | | | |
| | | GAGAGGAATGTCGGGGGGCGATGCTTTCGATATCTGGGGCC | | | | | | | MSGGDAFDIWGQGT | | | | | | |
| | | AAGGGACCACGGTCACCGTCTCCTCA | | | | | | | TVTVSS | | | | | | |
| ADI-81771 | 1286 | AGGGTCCTCGGTGAAAGTCTCCTGCAAGGCTTCTGGAGACA | | | | | | 7087 | EVQLVESGAEVKKP | | 7438 | 12 | | 12 | |
| | | CCTTCACCAGACATAGTATCAGCTGGGTGCGACAGGCCCCT | | | | | | | GSSVKVSCKASGDT | | | | | | |
| | | GGACAAGGCCTTGAGTGGATGGGAAGGATCATCCCTCTCCC | | | | | | | FTRHSISWVRQAPG | | | | | | |
| | | TGATATACCAAACTACGCTCAGAAGTTCCAGGGCAGAGTCA | | | | | | | QGLEWMGRIIPLPD | | | | | | |
| | | CGATTACCGCGGACAAATCCACGAACACAGCCTACATGGAG | | | | | | | IPNYAQKFQGRVTI | | | | | | |
| | | CTGAGCACCCTGAGATCTGACGACACGGCCGTCTATTACTG | | | | | | | TADKSTNTAYMELS | | | | | | |
| | | TGCGAGACACAATTCTCCGGTCTGGGGTCACTATGATAATA | | | | | | | TLRSDDTAVYYCAR | | | | | | |
| | | GTGGCCCCTTTGACTACTGGGGCCAGGGAACCCTGGTCACC | | | | | | | HNSPVWGHYDNSGP | | | | | | |
| | | GTCTCCTCA | | | | | | | FDYWGQGTLVTVSS | | | | | | |
| ADI-81865 | 1286 | GAGGTGCAGCTGGTGGAGTCTGGGGCTGAGGTGAAGAAACC | | | | | | 7088 | EVQLVESGAEVKKP | | 7439 | 12 | | 10 | |
| | | TGGGGCCTCAGTGAAGGTTTCCTGCAAGGCATCTGGACACA | | | | | | | GASVKVSCKASGHT | | | | | | |
| | | CCTTCGCCAACTACTATATACACTGGGTGCGACAGGCCCCT | | | | | | | FANYYIHWVRQAPG | | | | | | |
| | | GGACAAGGGCTTGAGTGGATGGGAATACCCAACCCTAGTGG | | | | | | | QGLEWMGIPNPSGG | | | | | | |
| | | TGGTAGCACAGCCTACGCACACAACTTCCAGGACAGAGTCA | | | | | | | STAYAHNFQDRVTM | | | | | | |
| | | CCATGACCGGGGACACGTCCACGAGCACAGTCTACATGGAG | | | | | | | TGDTSTSTVYMELS | | | | | | |
| | | CTCAGCAGCCTGAGATCTGAGGACACGGCCGTGTATTACTG | | | | | | | SLRSEDTAVYYCAR | | | | | | |
| | | TGCGAGAGGGGGTATAGTCCCAGATGCTAAGCAATACTTTG | | | | | | | GGIVPDAKQYFDYW | | | | | | |
| | | ACTACTGGGGCCAGGGAACCCTGGTCACCGTCTCCTCA | | | | | | | GQGTLVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81670 | 41 | IGHV3- | EVQLL | 5473 | FTLSSF | 5710 | WVRQTP | 448 | YITSSG | 6098 | RFTISR | 6406 | ARHLSPT | 6716 | WGQGTT |
| | | 48 | ESGGG | | SLN | | GKGLEW | | TTIYYA | | DNAKNL | | SYDFLSG | | VTVSS |
| | | | LAQPG | | | | VS | | DSVKG | | LFLQMN | | PDAFDI | | |
| | | | GSLRL | | | | | | | | SLRAED | | | | |
| | | | SCAAS | | | | | | | | TAVYYC | | | | |
| | | | G | | | | | | | | | | | | |
| ADI-81702 | 72 | IGHV1- | EVQLL | 5474 | YTFSHY | 5711 | WVRQAP | 433 | IINPSG | 6099 | RVTMTS | 6407 | ARDSVGV | 6717 | WGQGTT |
| | | 46 | ESGAE | | YIH | | GQGLEW | | VGTTYA | | DPSTST | | PAADALD | | VTVSS |
| | | | VKKPG | | | | MG | | QKFQG | | VYMELR | | I | | |
| | | | ASVKV | | | | | | | | SLTSED | | | | |
| | | | SCKAS | | | | | | | | TAVYYC | | | | |
| | | | G | | | | | | | | | | | | |
| ADI-81754 | 30 | IGHV3- | EVQLL | 5355 | FTVSVN | 5712 | WVRQAP | 5848 | VIYSGG | 689 | RFTVSR | 6408 | ARGDLGT | 6718 | WGQGTL |
| | | 66 | ESGGG | | YMT | | GRGPES | | STFYAD | | DHPKNT | | PRIDF | | VTVSS |
| | | | LVQPG | | | | VS | | SVKG | | LYLQMN | | | | |
| | | | GSLRL | | | | | | | | SLRVED | | | | |
| | | | SCAAS | | | | | | | | TAVYYC | | | | |
| | | | G | | | | | | | | | | | | |
| ADI-81855 | 33 | IGHV4- | EVQLL | 5365 | GSISGP | 5677 | WVRQPP | 444 | EIFHTG | 6053 | RATISI | 6367 | ARIMGAL | 6674 | WGQGTL |
| | | 4 | ESGPG | | NWWS | | GKGLEW | | NTKYNP | | DKSKNQ | | DY | | VTVSS |
| | | | LVKPS | | | | IG | | SLKS | | FSLKLS | | | | |
| | | | GTLSL | | | | | | | | SVTAAD | | | | |
| | | | TCAVS | | | | | | | | TAIYYC | | | | |
| | | | G | | | | | | | | | | | | |
| ADI-81573 | 35 | IGHV5- | QVQLV | 5475 | YSFISN | 5582 | WVRQKP | 5849 | IIWPGD | 5956 | QVTISV | 971 | ARRGSAW | 6719 | WGHGTL |
| | | 51 | QSGAE | | WIG | | GKGLEW | | SDTTYS | | DKSIST | | EIDY | | VTVSS |
| | | | VKRPG | | | | MG | | PSFQG | | AYLQWS | | | | |
| | | | ESLKI | | | | | | | | SLKASD | | | | |
| | | | SCQGS | | | | | | | | TAMYYC | | | | |
| | | | G | | | | | | | | | | | | |
| ADI-81614 | 71 | IGHV3- | QVQLV | 32 | FTFSRY | 5713 | WVRQAP | 443 | GISSDG | 6100 | RFTISR | 6409 | VKDIGPM | 6720 | WGQGTL |
| | | 64D | QSGGG | | SMD | | GKGLEY | | VTTYYA | | DNSKST | | TPVTTPE | | VTVSS |
| | | | LVQPG | | | | VS | | DSMKG | | LYLQMS | | S | | |
| | | | GSLRL | | | | | | | | SLRPED | | | | |
| | | | SCSAS | | | | | | | | TAVYYC | | | | |
| | | | G | | | | | | | | | | | | |
| ADI-81635 | 8 | IGHV1- | RCPLM | 5476 | YTFTSH | 5714 | WVRQAT | 491 | WTNPNS | 6101 | RVTMTR | 6410 | ARMSTTL | 6721 | WGQGTL |
| | | 8 | ESGAA | | DIN | | GQGLEW | | GATGYA | | NTSINT | | TNWFDP | | VTVSS |
| | | | VRKPG | | | | MG | | QKFQG | | AFMELS | | | | |
| | | | ASVKV | | | | | | | | SLRSDD | | | | |
| | | | SCKAS | | | | | | | | TAVYYC | | | | |
| | | | G | | | | | | | | | | | | |
| ADI-81652 | 25 | IGHV3- | QVQLV | 11 | FTFSDY | 5697 | WVRQAP | 431 | LIWYDG | 6102 | RFTISR | 6411 | ARAGLPT | 6722 | WGQGAL |
| | | 33 | ESGGG | | GMH | | GKGLEW | | YNKRYA | | DNSKNT | | DARALDG | | VTVSS |
| | | | VVQPG | | | | VA | | DSVKG | | LDLQMN | | SGNYYTF | | |
| | | | RSLRL | | | | | | | | SLRGED | | YFDS | | |
| | | | SCAAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81670 | 1287 | GAGGTGCAGCTGTTGGAGTCTGGGGGAGGCTTGGCACAGCC | | | | | | 7089 | EVQLLESGGGLAQP | | 7440 | 11 | | 10 | |
| | | TGGGGGGTCCCTGCGACTCTCCTGTGCAGCCTCTGGATTCA | | | | | | | GGSLRLSCAASGFT | | | | | | |
| | | CCCTCAGCAGTTTTAGTCTGAACTGGGTCCGCCAGACTCCA | | | | | | | LSSFSLNWVRQTPG | | | | | | |
| | | GGGAAGGGGCTGGAGTGGGTTTCATACATTACCAGTAGTGG | | | | | | | KGLEWVSYITSSGT | | | | | | |
| | | TACTACCATATACTACGCAGACTCTGTGAAGGGCCGATTCA | | | | | | | TIYYADSVKGRFTI | | | | | | |
| | | CCATCTCCAGAGACAATGCCAAGAACTTACTGTTTCTGCAA | | | | | | | SRDNAKNLLFLQMN | | | | | | |
| | | ATGAACAGCCTGAGAGCCGAGGACACGGCTGTCTATTACTG | | | | | | | SLRAEDTAVYYCAR | | | | | | |
| | | TGCGAGACATCTCTCACCAACGTCTTACGATTTTTTGAGTG | | | | | | | HLSPTSYDFLSGPD | | | | | | |
| | | GCCCAGATGCTTTTGATATATGGGGCCAAGGGACCACGGTC | | | | | | | AFDIWGQGTTVTVS | | | | | | |
| | | ACCGTCTCCTCA | | | | | | | S | | | | | | |
| ADI-81702 | 1287 | GAGGTGCAGCTGTTGGAGTCTGGGGCTGAGGTGAAGAAGCC | | | | | | 7090 | EVQLLESGAEVKKP | | 7441 | 11 | | 10 | |
| | | TGGGGCCTCAGTGAAGGTTTCCTGCAAGGCGTCTGGATACA | | | | | | | GASVKVSCKASGYT | | | | | | |
| | | CCTTCTCCCACTACTATATACACTGGGTGCGACAGGCCCCT | | | | | | | FSHYYIHWVRQAPG | | | | | | |
| | | GGACAGGGCCTTGAGTGGATGGGAATAATCAACCCTAGTGG | | | | | | | QGLEWMGIINPSGV | | | | | | |
| | | TGTTGGCACAACCTATGCACAGAAGTTCCAGGGCAGAGTCA | | | | | | | GTTYAQKFQGRVTM | | | | | | |
| | | CCATGACCAGTGACCCGTCCACGAGCACAGTCTACATGGAG | | | | | | | TSDPSTSTVYMELR | | | | | | |
| | | TTGAGAAGCCTGACATCTGAGGACACGGCCGTCTATTACTG | | | | | | | SLTSEDTAVYYCAR | | | | | | |
| | | TGCGAGAGATTCGGTAGGGGTTCCGGCTGCTGATGCCCTTG | | | | | | | DSVGVPAADALDIW | | | | | | |
| | | ATATCTGGGGCCAAGGGACCACGGTCACCGTCTCCTCA | | | | | | | GQGTTVTVSS | | | | | | |
| ADI-81754 | 1286 | GAGGTGCAGCTGTTGGAGTCTGGGGGAGGCTTGGTCCAGCC | | | | | | 7091 | EVQLLESGGGLVQP | | 7442 | 11 | | 10 | |
| | | TGGGGGGTCCCTGAGACTCTCCTGTGCAGCCTCTGGATTCA | | | | | | | GGSLRLSCAASGFT | | | | | | |
| | | CCGTCAGTGTCAACTACATGACCTGGGTCCGCCAGGCTCCA | | | | | | | VSVNYMTWVRQAPG | | | | | | |
| | | GGGAGGGGGCCGGAGTCGGTCTCAGTCATTTATAGCGGAGG | | | | | | | RGPESVSVIYSGGS | | | | | | |
| | | TAGCACATTCTACGCAGACTCCGTGAAGGGCAGATTCACCG | | | | | | | TFYADSVKGRFTVS | | | | | | |
| | | TCTCCAGAGACCATCCCAAGAACACCCTGTATCTTCAAATG | | | | | | | RDHPKNTLYLQMNS | | | | | | |
| | | AACAGCCTGAGAGTCGAGGACACGGCTGTGTATTATTGTGC | | | | | | | LRVEDTAVYYCARG | | | | | | |
| | | GAGAGGAGATCTCGGCACTCCAAGGATTGACTTCTGGGGCC | | | | | | | DLGTPRIDFWGQGT | | | | | | |
| | | AGGGAACCCTGGTCACCGTCTCCTCA | | | | | | | LVTVSS | | | | | | |
| ADI-81855 | 1286 | GAGGTGCAGCTGTTGGAGTCGGGCCCAGGACTGGTGAAGCC | | | | | | 7092 | EVQLLESGPGLVKP | | 7443 | 11 | | 9 | |
| | | TTCGGGGACCCTGTCCCTCACCTGCGCTGTCTCTGGTGGCT | | | | | | | SGTLSLTCAVSGGS | | | | | | |
| | | CCATCAGCGGTCCTAATTGGTGGAGTTGGGTCCGCCAGCCC | | | | | | | ISGPNWWSWVRQPP | | | | | | |
| | | CCAGGGAAGGGGCTGGAGTGGATTGGGGAAATCTTTCATAC | | | | | | | GKGLEWIGEIFHTG | | | | | | |
| | | TGGGAACACCAAGTACAACCCGTCCCTCAAGAGTCGAGCCA | | | | | | | NTKYNPSLKSRATI | | | | | | |
| | | CCATATCAATAGACAAGTCCAAGAACCAGTTCTCCCTGAAG | | | | | | | SIDKSKNQFSLKLS | | | | | | |
| | | CTGAGTTCTGTGACCGCCGCGGACACGGCCATTTATTACTG | | | | | | | SVTAADTAIYYCAR | | | | | | |
| | | TGCGAGAATAATGGGAGCCCTTGACTACTGGGGGCAGGGAA | | | | | | | IMGALDYWGQGTLV | | | | | | |
| | | CCCTGGTCACCGTCTCCTCA | | | | | | | TVSS | | | | | | |
| ADI-81573 | 6804 | CAGGTCCAGCTGGTGCAGTCTGGAGCAGAGGTGAAAAGGCC | | | | | | 7093 | QVQLVQSGAEVKRP | | 7444 | 10 | | 8 | |
| | | GGGGGAGTCTCTGAAGATCTCCTGTCAGGGTTCTGGATACA | | | | | | | GESLKISCQGSGYS | | | | | | |
| | | GCTTTATCAGCAACTGGATCGGCTGGGTGCGCCAGAAGCCC | | | | | | | FISNWIGWVRQKPG | | | | | | |
| | | GGCAAAGGCCTGGAGTGGATGGGAATCATCTGGCCTGGTGA | | | | | | | KGLEWMGIIWPGDS | | | | | | |
| | | CTCTGATACTACATACAGCCCGTCCTTCCAAGGCCAGGTCA | | | | | | | DTTYSPSFQGQVTI | | | | | | |
| | | CCATCTCAGTGGACAAGTCCATCAGCACCGCCTACCTGCAG | | | | | | | SVDKSISTAYLQWS | | | | | | |
| | | TGGAGCAGCCTGAAGGCCTCGGACACCGCCATGTATTACTG | | | | | | | SLKASDTAMYYCAR | | | | | | |
| | | TGCGAGACGAGGCAGTGCCTGGGAAATTGACTACTGGGGCC | | | | | | | RGSAWEIDYWGHGT | | | | | | |
| | | ACGGAACCCTGGTCACCGTCTCCTCA | | | | | | | LVTVSS | | | | | | |
| ADI-81614 | 1286 | CAGGTGCAGCTGGTGCAGTCTGGGGGAGGCTTGGTCCAGCC | | | | | | 7094 | QVQLVQSGGGLVQP | | 7445 | 10 | | 10 | |
| | | TGGGGGGTCCCTGAGACTCTCCTGTTCAGCCTCTGGATTCA | | | | | | | GGSLRLSCSASGFT | | | | | | |
| | | CCTTCAGTCGCTATTCTATGGACTGGGTCCGCCAGGCTCCA | | | | | | | FSRYSMDWVRQAPG | | | | | | |
| | | GGAAAGGGACTGGAATATGTTTCAGGTATAAGTAGTGATGG | | | | | | | KGLEYVSGISSDGV | | | | | | |
| | | TGTTACCACATACTACGCAGACTCAATGAAGGGCAGATTCA | | | | | | | TTYYADSMKGRFTI | | | | | | |
| | | CCATCTCCAGAGACAACTCCAAGAGCACTCTGTATCTTCAA | | | | | | | SRDNSKSTLYLQMS | | | | | | |
| | | ATGAGCAGTCTGAGACCTGAGGACACGGCTGTGTATTACTG | | | | | | | SLRPEDTAVYYCVK | | | | | | |
| | | TGTGAAAGATATCGGCCCGATGACTCCGGTGACTACGCCAG | | | | | | | DIGPMTPVTTPESW | | | | | | |
| | | AGTCCTGGGGCCAGGGAACCCTGGTCACCGTCTCCTCA | | | | | | | GQGTLVTVSS | | | | | | |
| ADI-81635 | 1286 | AGGTGCCCGCTGATGGAGTCTGGGGCTGCGGTGAGGAAGCC | | | | | | 7095 | RCPLMESGAAVRKP | | 7446 | 10 | | 8 | |
| | | TGGGGCCTCAGTGAAGGTCTCCTGCAAGGCCTCCGGATACA | | | | | | | GASVKVSCKASGYT | | | | | | |
| | | CCTTCACCAGTCATGATATCAACTGGGTGCGACAGGCCACT | | | | | | | FTSHDINWVRQATG | | | | | | |
| | | GGACAAGGGCTGGAGTGGATGGGATGGACAAACCCTAATAG | | | | | | | QGLEWMGWTNPNSG | | | | | | |
| | | TGGTGCCACAGGGTATGCACAGAAGTTCCAGGGCAGAGTCA | | | | | | | ATGYAQKFQGRVTM | | | | | | |
| | | CCATGACCAGGAATACCTCCATAAACACAGCCTTCATGGAG | | | | | | | TRNTSINTAFMELS | | | | | | |
| | | CTGAGCAGCCTGAGATCTGACGACACGGCCGTTTATTACTG | | | | | | | SLRSDDTAVYYCAR | | | | | | |
| | | TGCGAGAATGTCTACGACTTTAACGAACTGGTTCGACCCAT | | | | | | | MSTTLTNWFDPWGQ | | | | | | |
| | | GGGGCCAGGGAACCCTGGTCACCGTCTCCTCA | | | | | | | GTLVTVSS | | | | | | |
| ADI-81652 | 1285 | CAGGTGCAGCTGGTGGAGTCTGGGGGAGGCGTGGTCCAGCC | | | | | | 7096 | QVQLVESGGGVVQP | | 7447 | 10 | | 6 | |
| | | TGGGAGGTCCCTGAGACTCTCCTGTGCAGCGTCTGGATTCA | | | | | | | GRSLRLSCAASGFT | | | | | | |
| | | CCTTCAGTGACTATGGCATGCACTGGGTCCGCCAGGCTCCA | | | | | | | FSDYGMHWVRQAPG | | | | | | |
| | | GGCAAGGGGCTGGAGTGGGTAGCACTTATCTGGTATGATGG | | | | | | | KGLEWVALIWYDGY | | | | | | |
| | | ATATAATAAAAGATATGCAGACTCCGTGAAGGGCCGATTCA | | | | | | | NKRYADSVKGRFTI | | | | | | |
| | | CCATCTCCAGAGACAATTCCAAGAACACGCTGGATCTGCAA | | | | | | | SRDNSKNTLDLQMN | | | | | | |
| | | ATGAACAGCCTGAGAGGCGAGGACACGGCTGTCTATTACTG | | | | | | | SLRGEDTAVYYCAR | | | | | | |
| | | TGCGAGAGCGGGCCTCCCTACTGACGCCAGGGCTCTAGATG | | | | | | | AGLPTDARALDGSG | | | | | | |
| | | GTTCGGGGAATTATTACACTTTCTACTTTGACTCCTGGGGC | | | | | | | NYYTFYFDSWGQGA | | | | | | |
| | | CAGGGAGCCCTGGTCACCGTCTCCTCA | | | | | | | LVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81906 | 80 | IGHV3- | EVQLL | 5477 | FSVSVN | 5715 | WVRQAP | 437 | VIHRGG | 6103 | RFTISR | 6412 | ATSRVYY | 6723 | WGQGTT |
| | | 53 | ETGGG | | YMS | | GKGLEW | | STYYPD | | DNPQNT | | DSGGYPQ | | VTVSS |
| | | | LIQPG | | | | VS | | SVRG | | VYLQMN | | YHYYAMD | | |
| | | | GSLRL | | | | | | | | SLRAED | | V | | |
| | | | SCAAS | | | | | | | | TAVYYC | | | | |
| | | | G | | | | | | | | | | | | |
| ADI-81578 | 39 | IGHV1- | QVQLV | 50 | GTFSSY | 407 | WVRQAP | 5840 | KIIPML | 6104 | RATITA | 6413 | ARERGYS | 6724 | WGRGTL |
| | | 69 | QSGAE | | SIT | | GHGLEW | | GIADYA | | DKSTST | | DSGSVTY | | VTVSS |
| | | | VKKPG | | | | MG | | QKFQG | | AYMELS | | FDL | | |
| | | | SSVKV | | | | | | | | SLRSED | | | | |
| | | | SCKAS | | | | | | | | TAVYFC | | | | |
| | | | G | | | | | | | | | | | | |
| ADI-81856 | 34 | IGHV3- | EVQLL | 5478 | FTFSSY | 5716 | WVRQTP | 448 | SISGTS | 6105 | RFTISR | 6414 | ARDSHLL | 6725 | WGQGTT |
| | | 21 | ESGGG | | NMN | | GKGLEW | | SFISYA | | DNAKNS | | RFLTQPM | | VTVSS |
| | | | LVKPG | | | | VS | | DSVKG | | LYLQMN | | DV | | |
| | | | GSLRL | | | | | | | | SLRVED | | | | |
| | | | SCVAS | | | | | | | | TAVYYC | | | | |
| | | | G | | | | | | | | | | | | |
| ADI-81896 | 71 | IGHV3- | EVQLV | 5321 | FTFHDY | 5717 | WVRQAP | 437 | GISWNS | 6106 | RFAISR | 6415 | VRDGRGL | 6726 | WGQGTL |
| | | 9 | ESGGG | | AMH | | GKGLEW | | GNIEYA | | DNAKNF | | EPLGWFD | | VTVSS |
| | | | LVQPG | | | | VS | | DSVRG | | LYLQMN | | P | | |
| | | | RSLRL | | | | | | | | SMRVED | | | | |
| | | | SCAAS | | | | | | | | TALYYC | | | | |
| | | | G | | | | | | | | | | | | |
| ADI-81567 | 22 | IGHV1- | QVQLV | 50 | GTFSSS | 5718 | WVRQPP | 5850 | RIIPIL | 6107 | RVTITA | 6416 | ARDVGYS | 6727 | WGQGTL |
| | | 69 | QSGAE | | TIS | | GQGLEW | | DLADYA | | DKSTST | | GSGSSYY | | VPVSS |
| | | | VKKPG | | | | MG | | QKFQG | | AYMELS | | FDH | | |
| | | | SSVKV | | | | | | | | SLRSED | | | | |
| | | | SCKAS | | | | | | | | TALYYC | | | | |
| | | | G | | | | | | | | | | | | |
| ADI-81721 | 91 | IGHV5- | EVQLV | 5479 | YSFISN | 5582 | WVRQMP | 476 | IIWPGD | 6108 | QVTISA | 6417 | ARSSGTG | 6728 | WGQGTL |
| | | 51 | QSGAE | | WIG | | GKGLEW | | SDTRYS | | DKSIST | | YNDFDY | | VTVSS |
| | | | VKKPG | | | | MG | | PSFQG | | AYLQWN | | | | |
| | | | ESLKI | | | | | | | | SLKASD | | | | |
| | | | SCKTS | | | | | | | | TAMYYC | | | | |
| | | | G | | | | | | | | | | | | |
| ADI-81789 | 65 | IGHV3- | EVQLV | 5480 | FTFSRY | 331 | WVRQTT | 451 | AIGTAG | 6109 | RFTISR | 6418 | ARGDLAA | 6729 | WGRGTL |
| | | 13 | ESGGG | | DMH | | GKSLEW | | DAYYLD | | ENAKNS | | LDWHFDL | | VTVSS |
| | | | LAQPG | | | | VS | | SVKG | | LYLQMN | | | | |
| | | | GSLRL | | | | | | | | SLRAGD | | | | |
| | | | SCAAS | | | | | | | | TAVYFC | | | | |
| | | | G | | | | | | | | | | | | |
| ADI-81559 | 25 | IGHV3- | QVQLV | 5481 | FTFSSY | 5693 | WVRQAP | 431 | VIWSDG | 6110 | RFTISR | 6419 | ARDDGLP | 6730 | WGQGTL |
| | | 33 | ESGGG | | AIH | | GKGLEW | | SHKHYA | | DNSKNT | | TTYFDS | | VTVSS |
| | | | MVQPG | | | | VA | | DSVKG | | LYLQMD | | | | |
| | | | RSLRL | | | | | | | | SLRAED | | | | |
| | | | SCAAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81906 | 1287 | GAGGTGCAGCTGTTGGAGACTGGAGGAGGCTTGATCCAGCC | | | | | | 7097 | EVQLLETGGGLIQP | | 7448 | 10 | | 9 | |
| | | TGGGGGGTCCCTAAGACTCTCCTGTGCAGCCTCTGGATTCA | | | | | | | GGSLRLSCAASGFS | | | | | | |
| | | GCGTCAGTGTCAACTACATGAGCTGGGTCCGCCAGGCTCCA | | | | | | | VSVNYMSWVRQAPG | | | | | | |
| | | GGGAAGGGGCTGGAGTGGGTCTCAGTTATTCATAGGGGTGG | | | | | | | KGLEWVSVIHRGGS | | | | | | |
| | | TAGCACATACTACCCAGACTCCGTGAGGGGCCGATTCACCA | | | | | | | TYYPDSVRGRFTIS | | | | | | |
| | | TCTCCAGAGACAATCCCCAGAACACGGTGTATCTTCAAATG | | | | | | | RDNPQNTVYLQMNS | | | | | | |
| | | AACAGCCTGAGAGCCGAGGACACGGCCGTCTATTACTGTGC | | | | | | | LRAEDTAVYYCATS | | | | | | |
| | | GACGTCACGCGTTTACTATGACAGTGGTGGTTACCCGCAAT | | | | | | | RVYYDSGGYPQYHY | | | | | | |
| | | ACCATTACTACGCTATGGACGTCTGGGGCCAAGGGACCACG | | | | | | | YAMDVWGQGTTVTV | | | | | | |
| | | GTCACCGTCTCCTCA | | | | | | | SS | | | | | | |
| ADI-81578 | 1288 | TGGGTCCTCGGTGAAGGTCTCCTGCAAGGCTTCTGGAGGCA | | | | | | 7098 | QVQLVQSGAEVKKP | | 7449 | 9 | | 8 | |
| | | CCTTCAGCAGTTACAGTATCACCTGGGTGCGACAGGCCCCT | | | | | | | GSSVKVSCKASGGT | | | | | | |
| | | GGACATGGGCTTGAGTGGATGGGAAAGATCATCCCTATGCT | | | | | | | FSSYSITWVRQAPG | | | | | | |
| | | TGGTATAGCAGACTACGCACAGAAGTTCCAGGGCAGAGCTA | | | | | | | HGLEWMGKIIPMLG | | | | | | |
| | | CGATTACCGCGGACAAATCCACGAGCACAGCCTACATGGAG | | | | | | | IADYAQKFQGRATI | | | | | | |
| | | CTGAGCAGCCTGAGATCTGAGGACACGGCCGTGTATTTCTG | | | | | | | TADKSTSTAYMELS | | | | | | |
| | | TGCGAGAGAGCGGGGTTACTCTGATTCGGGGAGTGTTACGT | | | | | | | SLRSEDTAVYFCAR | | | | | | |
| | | ACTTCGATCTCTGGGGCCGTGGCACCCTGGTCACCGTCTCC | | | | | | | ERGYSDSGSVTYFD | | | | | | |
| | | TCA | | | | | | | LWGRGTLVTVSS | | | | | | |
| ADI-81856 | 1287 | GAGGTGCAGCTGTTGGAGTCTGGGGGAGGCCTGGTCAAGCC | | | | | | 7099 | EVQLLESGGGLVKP | | 7450 | 9 | | 8 | |
| | | TGGGGGGTCCCTGAGACTCTCCTGTGTAGCCTCTGGATTCA | | | | | | | GGSLRLSCVASGFT | | | | | | |
| | | CCTTCAGTAGTTATAACATGAACTGGGTCCGCCAGACTCCA | | | | | | | FSSYNMNWVRQTPG | | | | | | |
| | | GGGAAGGGGCTGGAGTGGGTCTCATCTATTAGTGGTACTAG | | | | | | | KGLEWVSSISGTSS | | | | | | |
| | | TAGTTTCATAAGCTACGCAGACTCAGTGAAGGGTCGATTCA | | | | | | | FISYADSVKGRFTI | | | | | | |
| | | CCATCTCCAGAGACAACGCCAAGAACTCACTCTATCTGCAA | | | | | | | SRDNAKNSLYLQMN | | | | | | |
| | | ATGAACAGCCTGAGAGTCGAGGACACGGCTGTGTATTACTG | | | | | | | SLRVEDTAVYYCAR | | | | | | |
| | | TGCGAGAGATTCCCACTTATTACGATTTTTGACCCAGCCTA | | | | | | | DSHLLRFLTQPMDV | | | | | | |
| | | TGGACGTCTGGGGCCAAGGGACCACGGTCACCGTCTCCTCA | | | | | | | WGQGTTVTVSS | | | | | | |
| ADI-81896 | 1286 | GAGGTGCAGCTGGTGGAGTCTGGGGGAGGCTTGGTACAGCC | | | | | | 7100 | EVQLVESGGGLVQP | | 7451 | 9 | | 8 | |
| | | TGGCAGGTCCCTGCGACTCTCCTGTGCAGCCTCTGGATTCA | | | | | | | GRSLRLSCAASGFT | | | | | | |
| | | CCTTTCATGATTATGCCATGCACTGGGTCCGCCAAGCTCCA | | | | | | | FHDYAMHWVRQAPG | | | | | | |
| | | GGGAAGGGCCTGGAGTGGGTTTCAGGTATTAGTTGGAATAG | | | | | | | KGLEWVSGISWNSG | | | | | | |
| | | TGGCAACATAGAATATGCGGACTCTGTGAGGGGCCGATTCG | | | | | | | NIEYADSVRGRFAI | | | | | | |
| | | CCATCTCCAGAGACAACGCCAAGAACTTCCTCTATCTGCAA | | | | | | | SRDNAKNFLYLQMN | | | | | | |
| | | ATGAACAGTATGAGAGTTGAGGACACGGCCTTGTATTATTG | | | | | | | SMRVEDTALYYCVR | | | | | | |
| | | TGTAAGAGATGGAAGAGGACTGGAACCATTAGGCTGGTTCG | | | | | | | DGRGLEPLGWFDPW | | | | | | |
| | | ACCCCTGGGGCCAGGGAACCCTGGTCACCGTCTCTTCA | | | | | | | GQGTLVTVSS | | | | | | |
| ADI-81567 | 6828 | TGGGTCCTCGGTGAAGGTCTCCTGCAAGGCTTCTGGAGGCA | | | | | | 7101 | QVQLVQSGAEVKKP | | 7452 | 8 | | 6 | |
| | | CCTTCAGCAGTTCTACAATCAGCTGGGTGCGACAGCCCCCT | | | | | | | GSSVKVSCKASGGT | | | | | | |
| | | GGACAAGGGCTTGAGTGGATGGGAAGGATCATCCCTATCCT | | | | | | | FSSSTISWVRQPPG | | | | | | |
| | | TGATTTGGCAGACTACGCACAGAAGTTTCAGGGCAGAGTCA | | | | | | | QGLEWMGRIIPILD | | | | | | |
| | | CCATTACCGCGGACAAATCCACGAGCACAGCCTACATGGAG | | | | | | | LADYAQKFQGRVTI | | | | | | |
| | | CTGAGCAGCCTGAGATCTGAGGACACGGCCCTCTATTACTG | | | | | | | TADKSTSTAYMELS | | | | | | |
| | | TGCGAGAGATGTGGGCTACTCTGGTTCGGGGAGTTCTTACT | | | | | | | SLRSEDTALYYCAR | | | | | | |
| | | ACTTTGACCACTGGGGCCAGGGAACCCTGGTCCCCGTCTCC | | | | | | | DVGYSGSGSSYYFD | | | | | | |
| | | TCA | | | | | | | HWGQGTLVPVSS | | | | | | |
| ADI-81721 | 1286 | GAGGTCCAGCTGGTGCAGTCTGGAGCAGAGGTGAAAAAGCC | | | | | | 7102 | EVQLVQSGAEVKKP | | 7453 | 8 | | 5 | |
| | | CGGGGAGTCTCTGAAGATCTCCTGTAAGACCTCTGGATACA | | | | | | | GESLKISCKTSGYS | | | | | | |
| | | GCTTTATCAGCAACTGGATCGGCTGGGTGCGCCAGATGCCC | | | | | | | FISNWIGWVRQMPG | | | | | | |
| | | GGGAAAGGCCTGGAGTGGATGGGGATCATCTGGCCTGGTGA | | | | | | | KGLEWMGIIWPGDS | | | | | | |
| | | CTCTGATACCAGATACAGCCCGTCCTTCCAAGGCCAGGTCA | | | | | | | DTRYSPSFQGQVTI | | | | | | |
| | | CCATCTCAGCCGACAAGTCCATTAGCACCGCCTACCTGCAA | | | | | | | SADKSISTAYLQWN | | | | | | |
| | | TGGAACAGCCTGAAGGCCTCGGACACCGCCATGTATTACTG | | | | | | | SLKASDTAMYYCAR | | | | | | |
| | | TGCGAGGAGTAGTGGAACTGGTTACAACGACTTTGACTACT | | | | | | | SSGTGYNDFDYWGQ | | | | | | |
| | | GGGGCCAGGGAACCCTGGTCACCGTCTCCTCA | | | | | | | GTLVTVSS | | | | | | |
| ADI-81789 | 1288 | GAGGTGCAGCTGGTGGAGTCTGGGGGAGGCCTGGCACAGCC | | | | | | 7103 | EVQLVESGGGLAQP | | 7454 | 8 | | 8 | |
| | | TGGGGGGTCCCTGAGACTCTCCTGTGCAGCCTCTGGATTCA | | | | | | | GGSLRLSCAASGFT | | | | | | |
| | | CCTTCAGTAGATACGACATGCACTGGGTCCGCCAGACTACA | | | | | | | FSRYDMHWVRQTTG | | | | | | |
| | | GGAAAAAGTCTGGAGTGGGTCTCAGCTATTGGTACTGCTGG | | | | | | | KSLEWVSAIGTAGD | | | | | | |
| | | TGACGCATACTATCTAGACTCCGTGAAGGGCCGATTCACCA | | | | | | | AYYLDSVKGRFTIS | | | | | | |
| | | TCTCCAGAGAAAATGCCAAGAACTCCTTGTATCTTCAAATG | | | | | | | RENAKNSLYLQMNS | | | | | | |
| | | AACAGCCTGAGAGCCGGGGACACGGCTGTGTATTTCTGTGC | | | | | | | LRAGDTAVYFCARG | | | | | | |
| | | AAGAGGGGATTTAGCAGCTCTCGACTGGCACTTCGATCTCT | | | | | | | DLAALDWHFDLWGR | | | | | | |
| | | GGGGCCGTGGCACCCTGGTCACCGTCTCCTCA | | | | | | | GTLVTVSS | | | | | | |
| ADI-81559 | 1286 | CAGGTGCAGCTGGTGGAGTCTGGGGGAGGCATGGTCCAGCC | | | | | | 7104 | QVQLVESGGGMVQP | | 7455 | 7 | | 7 | |
| | | TGGGAGGTCCCTGAGACTCTCCTGTGCAGCGTCTGGATTCA | | | | | | | GRSLRLSCAASGFT | | | | | | |
| | | CCTTCAGTAGCTACGCCATACACTGGGTCCGCCAGGCTCCA | | | | | | | FSSYAIHWVRQAPG | | | | | | |
| | | GGCAAGGGGCTGGAGTGGGTGGCAGTTATATGGTCTGATGG | | | | | | | KGLEWVAVIWSDGS | | | | | | |
| | | AAGTCATAAACACTATGCAGACTCCGTGAAGGGCCGATTCA | | | | | | | HKHYADSVKGRFTI | | | | | | |
| | | CCATCTCCAGAGACAATTCCAAGAACACCCTGTATCTGCAA | | | | | | | SRDNSKNTLYLQMD | | | | | | |
| | | ATGGACAGCCTGAGAGCCGAGGACACGGCTGTGTATTACTG | | | | | | | SLRAEDTAVYYCAR | | | | | | |
| | | TGCGAGAGATGACGGATTACCTACTACTTACTTTGACTCCT | | | | | | | DDGLPTTYFDSWGQ | | | | | | |
| | | GGGGCCAGGGAACCCTGGTCACCGTCTCCTCA | | | | | | | GTLVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81642 | 15 | IGHV4- | EVQLV | 5482 | YSISSG | 338 | WIRQPL | 487 | SIYHSG | 673 | RVTISL | 923 | ARDGQGY | 1173 | WGQGAL |
| | | 38-2 | ESGPG | | YSWG | | GKGLEW | | STYYKA | | DTSKNQ | | VPPRDNF | | VTVSS |
| | | | LVKAS | | | | IG | | SLKS | | FSLKLS | | DH | | |
| | | | ETLSL | | | | | | | | SVTAAD | | | | |
| | | | ACTVS | | | | | | | | TAVYYC | | | | |
| | | | G | | | | | | | | | | | | |
| ADI-81799 | 75 | IGHV4- | EVQLL | 5365 | GSISTS | 5719 | WVRQPP | 444 | EIYHTG | 6111 | RVTISV | 6420 | ARASEKG | 6731 | WGRGTL |
| | | 4 | ESGPG | | DWWS | | GKGLEW | | STNYNP | | DTSKNQ | | LLFGAYT | | VTVSS |
| | | | LVKPS | | | | IG | | SLKS | | FSLTLS | | YTYVDWY | | |
| | | | GTLSL | | | | | | | | SVTAAD | | FDI | | |
| | | | TCAVS | | | | | | | | TAMYYC | | | | |
| | | | G | | | | | | | | | | | | |
| ADI-81913 | 87 | IGHV3- | EVQLL | 5373 | FNFSNY | 5720 | WVRQAP | 431 | VLTYDG | 6112 | RFTISR | 821 | AKHPCSG | 6732 | WGQGTL |
| | | 30 | ESGGG | | GMH | | GKGLEW | | SNKYYA | | DNAKNT | | GSCYHEF | | VTVSS |
| | | | VVQPG | | | | VA | | GSVKG | | LYLQMN | | YFDY | | |
| | | | RSLRL | | | | | | | | SLRAED | | | | |
| | | | SCAAS | | | | | | | | TAVYYC | | | | |
| | | | G | | | | | | | | | | | | |
| ADI-81546 | 13 | IGHV3- | QVQLV | 10 | FTFSRY | 5721 | WVRQAP | 431 | NILYDG | 6113 | RFTISR | 777 | AKASGEY | 6733 | WGQGTL |
| | | 30 | QSGGG | | GMY | | GKGLEW | | SNKYYE | | DNSKNT | | CSGGSCY | | VTVSS |
| | | | VVQPG | | | | VA | | DSVKG | | LYLQMN | | NHYFDY | | |
| | | | RSLRL | | | | | | | | SLRAED | | | | |
| | | | SCAAS | | | | | | | | TAVYYC | | | | |
| | | | G | | | | | | | | | | | | |
| ADI-81563 | 11 | IGHV1- | QVQLV | 50 | GTFSSD | 5722 | WVRQAP | 433 | RVFPIL | 6114 | TVTITA | 6421 | AREGGTT | 6734 | WGHGTL |
| | | 69 | QSGAE | | SIS | | GQGLEW | | GIANYA | | DKSTST | | TEYFRH | | VTVSS |
| | | | VKKPG | | | | MG | | QKFQG | | AYMEMS | | | | |
| | | | SSVKV | | | | | | | | SLRSED | | | | |
| | | | SCKAS | | | | | | | | TAVYYC | | | | |
| | | | G | | | | | | | | | | | | |
| ADI-81596 | 56 | IGHV3- | EVQLV | 5483 | FIVSSN | 260 | WVRQAP | 437 | VIYPGG | 649 | RFTISR | 899 | ARDYGDY | 1149 | WGQGTL |
| | | 53 | ESGGG | | YMS | | GKGLEW | | STFYAD | | DTSKNT | | YFDY | | VTVSS |
| | | | LIQPG | | | | VS | | SVKG | | LYLQMN | | | | |
| | | | GSLRL | | | | | | | | SLRAED | | | | |
| | | | SCAAS | | | | | | | | TAVYYC | | | | |
| | | | E | | | | | | | | | | | | |
| ADI-81900 | 75 | IGHV3- | EVQLV | 5338 | FTFSSY | 5723 | WVRQTP | 5778 | LISSDG | 5991 | RFTISR | 789 | VKGKELR | 6735 | WGQGTL |
| | | 30 | ESGGG | | DIH | | GKGLEW | | SNKYYA | | DNSKNT | | AAAGRTY | | VTVSS |
| | | | VVQPG | | | | VA | | DSVKG | | LYLQMN | | RPLDY | | |
| | | | RSLRL | | | | | | | | SLRTED | | | | |
| | | | SCAAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81642 | 1285 | GAGGTGCAGCTGGTGGAGTCGGGCCCAGGACTGGTGAAGGC | | | | | | 7105 | EVQLVESGPGLVKA | | 7456 | 7 | | 7 | |
| | | TTCGGAGACCCTGTCCCTCGCCTGCACTGTCTCTGGTTACT | | | | | | | SETLSLACTVSGYS | | | | | | |
| | | CCATCAGCAGTGGTTATTCCTGGGGCTGGATCCGGCAGCCC | | | | | | | ISSGYSWGWIRQPL | | | | | | |
| | | CTAGGGAAGGGGCTGGAGTGGATTGGGAGTATCTATCATAG | | | | | | | GKGLEWIGSIYHSG | | | | | | |
| | | TGGGAGCACCTATTACAAAGCGTCCCTCAAGAGTCGAGTCA | | | | | | | STYYKASLKSRVTI | | | | | | |
| | | CCATATCACTAGACACGTCCAAGAACCAGTTCTCCCTGAAG | | | | | | | SLDTSKNQFSLKLS | | | | | | |
| | | CTGAGCTCTGTGACGGCCGCAGACACGGCCGTATATTACTG | | | | | | | SVTAADTAVYYCAR | | | | | | |
| | | TGCGAGAGATGGTCAAGGGTACGTACCACCGAGAGACAACT | | | | | | | DGQGYVPPRDNFDH | | | | | | |
| | | TTGACCACTGGGGCCAGGGAGCCCTGGTCACCGTCTCCTCA | | | | | | | WGQGALVTVSS | | | | | | |
| ADI-81799 | 1288 | GAGGTGCAGCTGTTGGAGTCGGGCCCAGGACTGGTGAAGCC | | | | | | 7106 | EVQLLESGPGLVKP | | 7457 | 7 | | 6 | |
| | | TTCGGGGACCCTGTCCCTCACCTGCGCTGTCTCTGGTGGCT | | | | | | | SGTLSLTCAVSGGS | | | | | | |
| | | CCATCAGCACTAGTGACTGGTGGAGTTGGGTCCGCCAGCCC | | | | | | | ISTSDWWSWVRQPP | | | | | | |
| | | CCAGGGAAGGGGCTGGAGTGGATTGGGGAAATCTATCATAC | | | | | | | GKGLEWIGEIYHTG | | | | | | |
| | | TGGGAGCACCAACTACAACCCGTCCCTCAAGAGTCGAGTCA | | | | | | | STNYNPSLKSRVTI | | | | | | |
| | | CCATCTCTGTAGACACGTCCAAGAACCAGTTCTCCCTGACA | | | | | | | SVDTSKNQFSLTLS | | | | | | |
| | | CTGAGCTCTGTGACCGCCGCGGACACGGCCATGTATTACTG | | | | | | | SVTAADTAMYYCAR | | | | | | |
| | | TGCGAGAGCTAGTGAGAAAGGTCTTCTCTTCGGGGCATACA | | | | | | | ASEKGLLFGAYTYT | | | | | | |
| | | CCTATACTTACGTGGACTGGTACTTCGATATCTGGGGCCGT | | | | | | | YVDWYFDIWGRGTL | | | | | | |
| | | GGCACCCTGGTCACCGTCTCCTCA | | | | | | | VTVSS | | | | | | |
| ADI-81913 | 1286 | TGGGAGGTCCCTGAGACTCTCCTGTGCAGCCTCTGGATTCA | | | | | | 7107 | EVQLLESGGGVVQP | | 7458 | 7 | | 6 | |
| | | ACTTCAGTAACTATGGCATGCACTGGGTCCGCCAGGCTCCA | | | | | | | GRSLRLSCAASGFN | | | | | | |
| | | GGCAAGGGGCTGGAGTGGGTGGCAGTTCTCACATATGATGG | | | | | | | FSNYGMHWVRQAPG | | | | | | |
| | | AAGTAATAAATACTATGCAGGCTCCGTGAAGGGCCGATTCA | | | | | | | KGLEWVAVLTYDGS | | | | | | |
| | | CCATCTCCAGAGACAATGCCAAGAACACGCTGTATCTGCAA | | | | | | | NKYYAGSVKGRFTI | | | | | | |
| | | ATGAACAGCCTGAGAGCTGAGGACACGGCTGTGTATTACTG | | | | | | | SRDNAKNTLYLQMN | | | | | | |
| | | TGCGAAACATCCATGTAGTGGTGGTAGCTGCTACCACGAAT | | | | | | | SLRAEDTAVYYCAK | | | | | | |
| | | TTTACTTTGACTACTGGGGCCAGGGAACCCTGGTCACCGTC | | | | | | | HPCSGGSCYHEFYF | | | | | | |
| | | TCCTCA | | | | | | | DYWGQGTLVTVSS | | | | | | |
| ADI-81546 | 1286 | CAGGTGCAGCTGGTGCAGTCTGGGGGAGGCGTGGTCCAGCC | | | | | | 7108 | QVQLVQSGGGVVQP | | 7459 | 6 | | 5 | |
| | | TGGGAGGTCCCTGAGACTCTCCTGTGCAGCCTCTGGATTCA | | | | | | | GRSLRLSCAASGFT | | | | | | |
| | | CCTTCAGTAGGTATGGCATGTACTGGGTCCGCCAGGCTCCA | | | | | | | FSRYGMYWVRQAPG | | | | | | |
| | | GGCAAGGGGCTGGAGTGGGTGGCAAATATATTATATGATGG | | | | | | | KGLEWVANILYDGS | | | | | | |
| | | AAGTAATAAATACTATGAAGACTCCGTGAAGGGCCGATTCA | | | | | | | NKYYEDSVKGRFTI | | | | | | |
| | | CCATCTCCAGAGACAATTCCAAGAACACGCTGTATCTGCAA | | | | | | | SRDNSKNTLYLQMN | | | | | | |
| | | ATGAACAGCCTGAGAGCTGAGGACACGGCTGTGTATTACTG | | | | | | | SLRAEDTAVYYCAK | | | | | | |
| | | TGCGAAAGCCTCAGGGGAATATTGTAGTGGTGGTAGCTGCT | | | | | | | ASGEYCSGGSCYNH | | | | | | |
| | | ACAACCACTACTTTGACTACTGGGGCCAGGGAACCCTGGTC | | | | | | | YFDYWGQGTLVTVS | | | | | | |
| | | ACCGTCTCCTCA | | | | | | | S | | | | | | |
| ADI-81563 | 6804 | CAGGTCCAGCTGGTGCAGTCTGGGGCTGAGGTGAAGAAGCC | | | | | | 7109 | QVQLVQSGAEVKKP | | 7460 | 6 | | 6 | |
| | | TGGGTCCTCGGTGAAGGTCTCCTGCAAGGCTTCTGGAGGCA | | | | | | | GSSVKVSCKASGGT | | | | | | |
| | | CCTTCAGCAGCGATAGTATCAGCTGGGTGCGACAGGCCCCT | | | | | | | FSSDSISWVRQAPG | | | | | | |
| | | GGACAAGGGCTTGAGTGGATGGGAAGGGTCTTCCCTATCCT | | | | | | | QGLEWMGRVFPILG | | | | | | |
| | | TGGTATAGCAAACTACGCACAGAAGTTCCAGGGCACAGTCA | | | | | | | IANYAQKFQGTVTI | | | | | | |
| | | CGATTACCGCGGACAAATCCACGAGCACAGCCTACATGGAA | | | | | | | TADKSTSTAYMEMS | | | | | | |
| | | ATGAGCAGCCTGAGATCTGAGGACACGGCCGTGTATTACTG | | | | | | | SLRSEDTAVYYCAR | | | | | | |
| | | TGCGAGAGAAGGTGGTACCACCACTGAATACTTCCGTCACT | | | | | | | EGGTTTEYFRHWGH | | | | | | |
| | | GGGGCCACGGCACCCTGGTCACCGTCTCCTCA | | | | | | | GTLVTVSS | | | | | | |
| ADI-81596 | 1286 | GAGGTGCAGCTGGTGGAGTCTGGAGGAGGCTTGATCCAGCC | | | | | | 7110 | EVQLVESGGGLIQP | | 7461 | 6 | | 5 | |
| | | TGGGGGGTCCCTGAGACTCTCCTGTGCAGCCTCTGAGTTCA | | | | | | | GGSLRLSCAASEFI | | | | | | |
| | | TCGTCAGTAGCAACTACATGAGCTGGGTCCGCCAGGCTCCA | | | | | | | VSSNYMSWVRQAPG | | | | | | |
| | | GGGAAGGGGCTGGAGTGGGTCTCAGTTATTTATCCCGGCGG | | | | | | | KGLEWVSVIYPGGS | | | | | | |
| | | TAGCACATTCTACGCAGACTCCGTGAAGGGCCGATTCACCA | | | | | | | TFYADSVKGRFTIS | | | | | | |
| | | TCTCCAGAGACACTTCCAAGAACACGCTGTATCTTCAAATG | | | | | | | RDTSKNTLYLQMNS | | | | | | |
| | | AACAGCCTGAGAGCCGAGGACACGGCCGTGTATTACTGTGC | | | | | | | LRAEDTAVYYCARD | | | | | | |
| | | GAGAGACTACGGTGACTACTACTTTGACTACTGGGGCCAGG | | | | | | | YGDYYFDYWGQGTL | | | | | | |
| | | GAACCCTGGTCACCGTCTCCTCA | | | | | | | VTVSS | | | | | | |
| ADI-81900 | 1286 | TGGGAGGTCCCTGAGACTCTCCTGTGCAGCCTCTGGATTCA | | | | | | 7111 | EVQLVESGGGVVQP | | 7462 | 6 | | 6 | |
| | | CCTTTAGTAGTTATGACATACACTGGGTCCGCCAGACTCCA | | | | | | | GRSLRLSCAASGFT | | | | | | |
| | | GGCAAGGGGCTGGAGTGGGTGGCACTTATCTCATCTGATGG | | | | | | | FSSYDIHWVRQTPG | | | | | | |
| | | AAGTAATAAATATTATGCAGACTCCGTGAAGGGCCGATTCA | | | | | | | KGLEWVALISSDGS | | | | | | |
| | | CCATCTCCAGAGACAATTCCAAGAACACGTTGTATCTGCAA | | | | | | | NKYYADSVKGRFTI | | | | | | |
| | | ATGAACAGCCTGAGAACTGAGGACACGGCTGTGTATTACTG | | | | | | | SRDNSKNTLYLQMN | | | | | | |
| | | TGTGAAAGGAAAGGAGTTGAGAGCAGCAGCTGGTAGGACCT | | | | | | | SLRTEDTAVYYCVK | | | | | | |
| | | ACCGTCCCCTTGACTACTGGGGCCAGGGAACCCTGGTCACC | | | | | | | GKELRAAAGRTYRP | | | | | | |
| | | GTCTCCTCA | | | | | | | LDYWGQGTLVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81581 | 42 | IGHV3- | QVQLV | 5484 | FTFGDY | 5724 | WVRQVP | 436 | GINWNG | 6115 | RFTISR | 6422 | ARGGAIF | 6736 | WGQGTT |
| | | 20 | QSGGG | | GMN | | GKGLEW | | GRTGYA | | DNAKNS | | RYFDLLS | | VTVSS |
| | | | VVRPG | | | | VS | | DPVKG | | LYLQMN | | SEGMDV | | |
| | | | GSLRL | | | | | | | | SLRAED | | | | |
| | | | SCAAS | | | | | | | | TALYYC | | | | |
| | | | G | | | | | | | | | | | | |
| ADI-81615 | 72 | IGHV1- | QVQLQ | 86 | GTFSSY | 356 | WVRQAP | 433 | RIIPLL | 765 | RVTITA | 832 | ARENCFG | 1270 | WGQGTT |
| | | 69 | QSGAE | | SIS | | GQGLEW | | GIPNYA | | DKSTST | | TSCSTPW | | VTVSS |
| | | | VKKPG | | | | MG | | QKMQG | | AYMELS | | GAFDI | | |
| | | | SSVKV | | | | | | | | SLRSED | | | | |
| | | | SCKAS | | | | | | | | TAVYYC | | | | |
| | | | G | | | | | | | | | | | | |
| ADI-81904 | 78 | IGHV4- | QVQLL | 5485 | YSISSG | 5725 | WVRQPP | 444 | SIYHSG | 6116 | RVTISV | 6423 | ARVGAYC | 6737 | WGQGTL |
| | | 38-2 | ESGPG | | YYWG | | GKGLEW | | RTYYNP | | DTSKNQ | | GGGHCYP | | VTVSS |
| | | | LVKPS | | | | IG | | SLQS | | FSLKLT | | EDKQSPD | | |
| | | | ETLSL | | | | | | | | SVTAAD | | Y | | |
| | | | TCGVS | | | | | | | | TAVYYC | | | | |
| | | | G | | | | | | | | | | | | |
| ADI-81818 | 92 | IGHV4- | EVQLL | 5335 | GSISSY | 5726 | WIRQPP | 445 | YTSYSG | 6117 | RVTISV | 6423 | AADYYDS | 6738 | WGQGTT |
| | | 59 | ESGPG | | YWT | | GKGLEW | | STNYNP | | DTSKNQ | | TGYHYGM | | VTVSS |
| | | | LVKPS | | | | IG | | SLKS | | FSLKLT | | DV | | |
| | | | ETLSL | | | | | | | | SVTAAD | | | | |
| | | | TCTVS | | | | | | | | TAVYYC | | | | |
| | | | G | | | | | | | | | | | | |
| ADI-81540 | 8 | IGHV5- | QVQLV | 174 | YSFTHY | 5727 | WVRQMP | 476 | IIYPGD | 635 | QVTISA | 6424 | ARQGTTV | 1134 | WGQGTL |
| | | 51 | QSGAE | | WIG | | GKGLEW | | SDTRYS | | DKSIST | | GHFDY | | VTVSS |
| | | | VKKPG | | | | MG | | PSFQG | | AYLQWS | | | | |
| | | | ESLKI | | | | | | | | GLKASD | | | | |
| | | | SCKGS | | | | | | | | TAMYYC | | | | |
| | | | G | | | | | | | | | | | | |
| ADI-81880 | 57 | IGHV3- | EVQLV | 5321 | FTFDDY | 214 | WVRQAP | 437 | GISWNS | 6118 | RFTISR | 6393 | AKDLSIN | 6739 | WGQGTT |
| | | 9 | ESGGG | | AMH | | GKGLEW | | ASIGYA | | DNAKNS | | WGSPLSG | | VTVSS |
| | | | LVQPG | | | | VS | | DSVKG | | LYLQMN | | MDV | | |
| | | | RSLRL | | | | | | | | SLRAED | | | | |
| | | | SCAAS | | | | | | | | TAFYYC | | | | |
| | | | G | | | | | | | | | | | | |
| ADI-81568 | 31 | IGHV3- | QVQLV | 10 | FTFSSY | 262 | WVRQAP | 431 | VIWYDG | 679 | RFTISR | 777 | ARARGWG | 6740 | WGQGTL |
| | | 33 | QSGGG | | GMH | | GKGLEW | | SNKYYA | | DNSKNT | | VGATIDY | | VTVSS |
| | | | VVQPG | | | | VA | | DSVKG | | LYLQMN | | | | |
| | | | RSLRL | | | | | | | | SLRAED | | | | |
| | | | SCAAS | | | | | | | | TAVYYC | | | | |
| | | | G | | | | | | | | | | | | |
| ADI-81658 | 31 | IGHV3- | RGAAG | 5486 | FTFSSF | 5728 | GVRQAP | 5851 | IITTSG | 6119 | RFTISK | 6425 | DKSEAYG | 6741 | WAQGTL |
| | | 23 | GVWGR | | PMS | | GKGLEC | | G- | | DTCKNT | | TGWYGKN | | VTVSS |
| | | | LGTAR | | | | VS | | TTYPDS | | LYGQMN | | DY | | |
| | | | GSLRL | | | | | | VKG | | SLRAED | | | | |
| | | | SCAAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81581 | 1287 | CAGGTGCAGCTGGTGCAGTCTGGGGGAGGTGTGGTACGGCC | | | | | | 7112 | QVQLVQSGGGVVRP | | 7463 | 5 | | 5 | |
| | | TGGGGGGTCCCTGAGACTCTCCTGTGCAGCCTCTGGATTCA | | | | | | | GGSLRLSCAASGFT | | | | | | |
| | | CCTTTGGTGATTATGGCATGAACTGGGTCCGCCAAGTTCCA | | | | | | | FGDYGMNWVRQVPG | | | | | | |
| | | GGGAAGGGGCTGGAGTGGGTCTCTGGTATTAATTGGAATGG | | | | | | | KGLEWVSGINWNGG | | | | | | |
| | | TGGTAGGACAGGGTATGCAGACCCTGTGAAGGGCCGATTCA | | | | | | | RTGYADPVKGRFTI | | | | | | |
| | | CCATCTCCAGAGACAACGCCAAGAACTCCCTATATCTGCAA | | | | | | | SRDNAKNSLYLQMN | | | | | | |
| | | ATGAACAGTCTGAGAGCCGAGGACACGGCCTTGTACTACTG | | | | | | | SLRAEDTALYYCAR | | | | | | |
| | | TGCGAGAGGGGGGGCCATATTCCGATATTTTGACTTGTTAT | | | | | | | GGAIFRYFDLLSSE | | | | | | |
| | | CCTCCGAGGGGATGGACGTCTGGGGCCAAGGGACCACGGTC | | | | | | | GMDVWGQGTTVTVS | | | | | | |
| | | ACCGTCTCCTCA | | | | | | | S | | | | | | |
| ADI-81615 | 1287 | TGGGTCCTCGGTGAAGGTCTCCTGCAAGGCTTCTGGAGGCA | | | | | | 7113 | QVQLQQSGAEVKKP | | 7464 | 5 | | 4 | |
| | | CCTTCAGCAGCTATTCTATCAGCTGGGTGCGACAGGCCCCT | | | | | | | GSSVKVSCKASGGT | | | | | | |
| | | GGACAAGGACTTGAGTGGATGGGAAGGATCATCCCTCTCCT | | | | | | | FSSYSISWVRQAPG | | | | | | |
| | | TGGTATACCAAACTACGCACAGAAGATGCAGGGCAGAGTCA | | | | | | | QGLEWMGRIIPLLG | | | | | | |
| | | CGATTACCGCGGACAAATCAACGAGCACAGCCTACATGGAG | | | | | | | IPNYAQKMQGRVTI | | | | | | |
| | | CTGAGCAGCCTGAGATCTGAGGACACGGCCGTGTATTACTG | | | | | | | TADKSTSTAYMELS | | | | | | |
| | | TGCGAGAGAAAATTGTTTTGGTACCTCCTGCTCTACCCCGT | | | | | | | SLRSEDTAVYYCAR | | | | | | |
| | | GGGGTGCTTTTGATATCTGGGGCCAAGGGACCACGGTCACC | | | | | | | ENCFGTSCSTPWGA | | | | | | |
| | | GTCTCCTCA | | | | | | | FDIWGQGTTVTVSS | | | | | | |
| ADI-81904 | 1286 | CAGGTGCAGCTGCTGGAGTCCGGCCCAGGACTGGTGAAGCC | | | | | | 7114 | QVQLLESGPGLVKP | | 7465 | 5 | | 5 | |
| | | TTCGGAGACCCTGTCCCTCACCTGCGGTGTCTCTGGTTACT | | | | | | | SETLSLTCGVSGYS | | | | | | |
| | | CCATCAGCAGTGGTTACTACTGGGGCTGGGTCCGGCAGCCC | | | | | | | ISSGYYWGWVRQPP | | | | | | |
| | | CCAGGGAAGGGGCTGGAGTGGATTGGGAGTATCTATCATAG | | | | | | | GKGLEWIGSIYHSG | | | | | | |
| | | TGGGAGGACCTACTACAACCCGTCCCTCCAGAGTCGAGTCA | | | | | | | RTYYNPSLQSRVTI | | | | | | |
| | | CCATATCAGTTGACACGTCCAAGAACCAGTTCTCCCTGAAA | | | | | | | SVDTSKNQFSLKLT | | | | | | |
| | | CTGACCTCTGTGACCGCCGCAGACACGGCCGTGTATTACTG | | | | | | | SVTAADTAVYYCAR | | | | | | |
| | | TGCGAGAGTGGGGGCGTATTGTGGTGGTGGTCACTGCTACC | | | | | | | VGAYCGGGHCYPED | | | | | | |
| | | CCGAAGACAAACAATCACCGGACTACTGGGGCCAGGGAACC | | | | | | | KQSPDYWGQGTLVT | | | | | | |
| | | CTGGTCACCGTCTCCTCA | | | | | | | VSS | | | | | | |
| ADI-81818 | 1287 | GAGGTGCAGCTGTTGGAGTCGGGCCCAGGACTGGTGAAGCC | | | | | | 7115 | EVQLLESGPGLVKP | | 7466 | 4 | | 4 | |
| | | TTCGGAGACCCTGTCCCTCACCTGCACTGTCTCTGGTGGCT | | | | | | | SETLSLTCTVSGGS | | | | | | |
| | | CCATCAGTAGTTACTACTGGACCTGGATCCGGCAGCCCCCA | | | | | | | ISSYYWTWIRQPPG | | | | | | |
| | | GGGAAGGGACTGGAGTGGATTGGATATACCTCTTACAGTGG | | | | | | | KGLEWIGYTSYSGS | | | | | | |
| | | GAGCACCAACTACAACCCCTCCCTCAAGAGTCGAGTCACCA | | | | | | | TNYNPSLKSRVTIS | | | | | | |
| | | TATCAGTAGACACGTCCAAGAACCAGTTCTCCCTGAAGCTG | | | | | | | VDTSKNQFSLKLTS | | | | | | |
| | | ACCTCTGTGACCGCTGCGGACACGGCCGTCTATTACTGTGC | | | | | | | VTAADTAVYYCAAD | | | | | | |
| | | GGCTGATTACTATGATAGTACTGGTTACCACTACGGTATGG | | | | | | | YYDSTGYHYGMDVW | | | | | | |
| | | ACGTCTGGGGCCAAGGGACCACGGTCACCGTCTCCTCA | | | | | | | GQGTTVTVSS | | | | | | |
| ADI-81540 | 1286 | CAGGTCCAGCTGGTGCAGTCTGGAGCAGAGGTGAAAAAGCC | | | | | | 7116 | QVQLVQSGAEVKKP | | 7467 | 3 | | 2 | |
| | | CGGGGAGTCTCTGAAGATCTCCTGTAAGGGTTCTGGATACA | | | | | | | GESLKISCKGSGYS | | | | | | |
| | | GCTTTACCCACTACTGGATCGGCTGGGTGCGCCAGATGCCC | | | | | | | FTHYWIGWVRQMPG | | | | | | |
| | | GGGAAAGGCCTGGAGTGGATGGGGATCATCTATCCTGGTGA | | | | | | | KGLEWMGIIYPGDS | | | | | | |
| | | CTCTGATACCAGATACAGCCCGTCCTTCCAAGGCCAGGTCA | | | | | | | DTRYSPSFQGQVTI | | | | | | |
| | | CCATCTCAGCCGACAAGTCCATCAGCACCGCCTACCTGCAG | | | | | | | SADKSISTAYLQWS | | | | | | |
| | | TGGAGCGGCCTGAAGGCCTCGGACACCGCCATGTATTACTG | | | | | | | GLKASDTAMYYCAR | | | | | | |
| | | TGCGAGACAGGGCACGACAGTAGGACACTTTGACTACTGGG | | | | | | | QGTTVGHFDYWGQG | | | | | | |
| | | GCCAGGGAACCCTGGTCACCGTCTCCTCA | | | | | | | TLVTVSS | | | | | | |
| ADI-81880 | 1287 | TGGCAGGTCCCTGAGACTCTCCTGTGCAGCCTCTGGATTCA | | | | | | 7117 | EVQLVESGGGLVQP | | 7468 | 2 | | 2 | |
| | | CCTTTGATGATTATGCCATGCACTGGGTCCGGCAAGCTCCA | | | | | | | GRSLRLSCAASGFT | | | | | | |
| | | GGGAAGGGCCTGGAGTGGGTCTCAGGTATTAGTTGGAACAG | | | | | | | FDDYAMHWVRQAPG | | | | | | |
| | | TGCTAGCATAGGCTATGCGGACTCTGTGAAGGGCCGATTCA | | | | | | | KGLEWVSGISWNSA | | | | | | |
| | | CCATCTCCAGAGACAACGCCAAGAACTCCCTGTATCTGCAA | | | | | | | SIGYADSVKGRFTI | | | | | | |
| | | ATGAACAGTCTGAGAGCTGAGGACACGGCCTTTTATTACTG | | | | | | | SRDNAKNSLYLQMN | | | | | | |
| | | TGCAAAAGATTTATCTATAAACTGGGGATCCCCACTCTCCG | | | | | | | SLRAEDTAFYYCAK | | | | | | |
| | | GTATGGACGTCTGGGGCCAAGGGACCACGGTCACCGTCTCC | | | | | | | DLSINWGSPLSGMD | | | | | | |
| | | TCA | | | | | | | VWGQGTTVTVSS | | | | | | |
| ADI-81568 | 1286 | CAGGTGCAGCTGGTGCAGTCTGGGGGAGGCGTGGTCCAGCC | | | | | | 7118 | QVQLVQSGGGVVQP | | 7469 | 0 | | 0 | |
| | | TGGGAGGTCCCTGAGACTCTCCTGTGCAGCGTCTGGATTCA | | | | | | | GRSLRLSCAASGFT | | | | | | |
| | | CCTTCAGTAGCTATGGCATGCACTGGGTCCGCCAGGCTCCA | | | | | | | FSSYGMHWVRQAPG | | | | | | |
| | | GGCAAGGGGCTGGAGTGGGTGGCAGTTATATGGTATGATGG | | | | | | | KGLEWVAVIWYDGS | | | | | | |
| | | AAGTAATAAATACTATGCAGACTCCGTGAAGGGCCGATTCA | | | | | | | NKYYADSVKGRFTI | | | | | | |
| | | CCATCTCCAGAGACAATTCCAAGAACACGCTGTATCTGCAA | | | | | | | SRDNSKNTLYLQMN | | | | | | |
| | | ATGAACAGCCTGAGAGCCGAGGACACGGCTGTGTATTACTG | | | | | | | SLRAEDTAVYYCAR | | | | | | |
| | | TGCGAGAGCTCGAGGCTGGGGAGTGGGAGCTACTATTGACT | | | | | | | ARGWGVGATIDYWG | | | | | | |
| | | ACTGGGGCCAGGGAACCCTGGTCACCGTCTCCTCA | | | | | | | QGTLVTVSS | | | | | | |
| ADI-81658 | 6829 | CGAGGTGCAGCTGGTGGAGTCTGGGGGAGACTTGGTACAGC | | | | | | 7119 | RGAAGGVWGRLGTA | | 7470 | 33 | | 19 | |
| | | CAGGGGGTCCCTGAGACTCTCCTGCGCAGCCTCTGGATTTA | | | | | | | RGSLRLSCAASGFT | | | | | | |
| | | CCTTTAGCAGTTTTCCCATGAGCGGGGTCCGCCAGGCTCCA | | | | | | | FSSFPMSGVRQAPG | | | | | | |
| | | GGGAAGGGGCTAGAGTGTGTTTCAATTATCACTACTAGTGG | | | | | | | KGLECVSIITTSGG | | | | | | |
| | | TGGTACCACATATCCCGACTCCGTTAAGGGCCGGTTCACCA | | | | | | | TTYPDSVKGRFTIS | | | | | | |
| | | TCTCCAAAGACACTTGTAAGAACACCCTGTATGGGCAAATG | | | | | | | KDTCKNTLYGQMNS | | | | | | |
| | | AACAGCCTGAGAGCCGAGGACACGGCCGTATATTACTGTGA | | | | | | | LRAEDTAVYYCDKS | | | | | | |
| | | CAAGTCCGAGGCGTATGGCACTGGCTGGTACGGAAAAAATG | | | | | | | EAYGTGWYGKNDYW | | | | | | |
| | | ACTACTGGGCCCAGGGAACCCTGGTCACAGTTTCATCA | | | | | | | AQGTLVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81785 | 61 | IGHV3- | EVQLV | 5487 | FTFANY | 5729 | WVRQAP | 431 | AVDSDG | 6120 | RFTISR | 6426 | ARTRHYD | 6742 | WGTGTL |
| | | NL1 | ESGGG | | YMT | | GKGLEW | | SRTYYA | | DNAKST | | GLDH | | VTVSS |
| | | | LVQTG | | | | VA | | DSVRG | | LYLQMN | | | | |
| | | | GSLRL | | | | | | | | NLNSED | | | | |
| | | | SCASS | | | | | | | | TAVYYC | | | | |
| | | | G | | | | | | | | | | | | |
| ADI-81631 | 4 | IGHV3- | LGAAG | 5488 | FTVSSN | 281 | WVRQAP | 5852 | FIYSGG | 6121 | RFPISR | 6427 | VREIYGC | 6743 | WGQGTL |
| | | 53 | AVWGR | | YMS | | GKGVEW | | STFYRD | | DKSKNT | | TSCQHGI | | VTVSS |
| | | | LGPAG | | | | VS | | SVKG | | LYLQMN | | PRDH | | |
| | | | GSLRI | | | | | | | | SLRPED | | | | |
| | | | SCAAS | | | | | | | | TAVYYC | | | | |
| | | | G | | | | | | | | | | | | |
| ADI-81841 | 19 | IGHV4- | EVQLL | 5489 | GSVGSG | 5730 | WIRHHP | 5853 | YIHYSG | 6122 | RVTISL | 6428 | ARAPCSG | 6744 | WGQGTL |
| | | 31 | ESGPG | | DFYWN | | GKGLEW | | STFYNP | | DTAKNQ | | GGCYYGS | | VTVSS |
| | | | LVKPS | | | | IG | | SLKN | | LSLKLG | | LRGAFDY | | |
| | | | QTLSL | | | | | | | | SVTAAD | | | | |
| | | | TCSVS | | | | | | | | TAIYYC | | | | |
| | | | G | | | | | | | | | | | | |
| ADI-81669 | 40 | IGHV3- | EVQLL | 5490 | FPFNTH | 5731 | WVRLAP | 5854 | VISYDG | 6123 | RFTIAR | 6429 | GRARSTF | 6745 | GGQGTT |
| | | 30 | ESGGG | | GLN | | GKGLQW | | NYKYYA | | DNSKNT | | CTSSSCY | | VTVSS |
| | | | VVQPG | | | | VA | | DSVKG | | MYLQMN | | SAAFDF | | |
| | | | RSLRL | | | | | | | | SLRPDD | | | | |
| | | | SCATS | | | | | | | | TAVYYC | | | | |
| | | | G | | | | | | | | | | | | |
| ADI-81787 | 63 | IGHV3- | EVQLV | 5350 | FTFSSY | 199 | WVRQTP | 5855 | VILDDG | 6124 | RFTISR | 6430 | ARDSSTA | 6746 | WGQGTL |
| | | 30 | QSGGG | | AMH | | DKGLEW | | RGKFYA | | DNLKNT | | DSLVSWF | | VTVSS |
| | | | VVQPG | | | | VA | | DSVKG | | LYLQMN | | DP | | |
| | | | RSLRL | | | | | | | | TVGVED | | | | |
| | | | SCAAS | | | | | | | | TAVYYC | | | | |
| | | | G | | | | | | | | | | | | |
| ADI-81788 | 64 | IGHV4- | LGAVG | 5491 | HSIISG | 5732 | WIRQLP | 5856 | YMYYSG | 6125 | RLTISI | 6431 | VRESIVV | 6747 | WGQGTT |
| | | 31 | ESGPG | | SYYSS | | GKGLEW | | STYYNP | | DTFKNQ | | APAAGTY | | VTVSS |
| | | | LVKPS | | | | IG | | SLKS | | YSLKLS | | PSYYGID | | |
| | | | QTLSL | | | | | | | | SVTAAD | | V | | |
| | | | TCTVS | | | | | | | | TAVYYC | | | | |
| | | | G | | | | | | | | | | | | |
| ADI-81566 | 30 | IGHV1- | QVQLV | 50 | DTFSNY | 5733 | WVRQAP | 433 | RIIPIF | 6126 | RVTISA | 6432 | ARGRTDD | 6748 | WGQGTL |
| | | 69 | QSGAE | | AIT | | GQGLEW | | GITNIA | | DKSTGT | | YLDS | | VTVSS |
| | | | VKKPG | | | | MG | | QKFQG | | TYMELS | | | | |
| | | | SSVKV | | | | | | | | SLRSED | | | | |
| | | | SCKAS | | | | | | | | TAVYFC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81785 | 6830 | GAGGTGCAGCTGGTGGAATCTGGAGGAGGATTGGTGCAAAC | | | | | | 7120 | EVQLVESGGGLVQT | | 7471 | 30 | | 19 | |
| | | TGGGGGCTCTCTGAGACTCTCCTGTGCATCCTCGGGATTCA | | | | | | | GGSLRLSCASSGFT | | | | | | |
| | | CCTTCGCGAATTACTATATGACCTGGGTCCGACAGGCTCCA | | | | | | | FANYYMTWVRQAPG | | | | | | |
| | | GGCAAGGGACTGGAGTGGGTGGCCGCTGTTGATAGCGACGG | | | | | | | KGLEWVAAVDSDGS | | | | | | |
| | | CAGTAGGACATACTATGCAGACTCCGTAAGGGGCCGATTCA | | | | | | | RTYYADSVRGRFTI | | | | | | |
| | | CCATCTCCAGAGACAACGCCAAGAGCACCTTATATCTGCAA | | | | | | | SRDNAKSTLYLQMN | | | | | | |
| | | ATGAATAATCTGAATTCTGAAGACACGGCCGTGTATTACTG | | | | | | | NLNSEDTAVYYCAR | | | | | | |
| | | TGCCCGAACTAGGCACTACGACGGCCTGGATCACTGGGGCA | | | | | | | TRHYDGLDHWGTGT | | | | | | |
| | | CAGGGACCCTGGTCACCGTCTCCTCA | | | | | | | LVTVSS | | | | | | |
| ADI-81631 | 1286 | TGGGGGGTCCCTGAGAATTTCTTGTGCAGCTTCTGGGTTCA | | | | | | 7121 | LGAAGAVWGRLGPA | | 7472 | 18 | | 13 | |
| | | CCGTCAGTAGCAACTACATGAGCTGGGTCCGCCAGGCTCCA | | | | | | | GGSLRISCAASGFT | | | | | | |
| | | GGAAAGGGGGTGGAGTGGGTTTCATTTATTTATAGCGGTGG | | | | | | | VSSNYMSWVRQAPG | | | | | | |
| | | TAGCACATTCTACCGAGATTCCGTAAAGGGCCGATTCCCCA | | | | | | | KGVEWVSFIYSGGS | | | | | | |
| | | TCTCCAGAGACAAATCCAAGAACACACTGTATCTTCAAATG | | | | | | | TFYRDSVKGRFPIS | | | | | | |
| | | AACAGCCTGAGACCTGAGGACACGGCTGTGTATTACTGTGT | | | | | | | RDKSKNTLYLQMNS | | | | | | |
| | | GAGAGAAATATATGGTTGTACCAGCTGCCAACACGGGATCC | | | | | | | LRPEDTAVYYCVRE | | | | | | |
| | | CACGGGACCACTGGGGCCAGGGAACCCTGGTCACCGTTTCC | | | | | | | IYGCTSCQHGIPRD | | | | | | |
| | | TCA | | | | | | | HWGQGTLVTVSS | | | | | | |
| ADI-81841 | 1286 | GAGGTGCAGCTGTTGGAGTCGGGCCCAGGACTGGTGAAGCC | | | | | | 7122 | EVQLLESGPGLVKP | | 7473 | 17 | | 15 | |
| | | TTCACAGACCCTGTCCCTCACCTGCTCTGTCTCTGGTGGCT | | | | | | | SQTLSLTCSVSGGS | | | | | | |
| | | CCGTCGGCAGTGGTGATTTCTACTGGAACTGGATCCGCCAC | | | | | | | VGSGDFYWNWIRHH | | | | | | |
| | | CACCCGGGGAAGGGCCTGGAGTGGATTGGGTACATCCATTA | | | | | | | PGKGLEWIGYIHYS | | | | | | |
| | | CAGTGGGAGCACCTTCTACAACCCGTCCCTCAAGAATCGAG | | | | | | | GSTFYNPSLKNRVT | | | | | | |
| | | TTACCATATCACTGGACACGGCTAAGAACCAGCTCTCCCTG | | | | | | | ISLDTAKNQLSLKL | | | | | | |
| | | AAGCTGGGCTCTGTGACTGCCGCGGACACGGCCATATATTA | | | | | | | GSVTAADTAIYYCA | | | | | | |
| | | CTGTGCGCGAGCCCCCTGTAGTGGTGGTGGCTGCTATTACG | | | | | | | RAPCSGGGCYYGSL | | | | | | |
| | | GCTCTTTGCGCGGGGCTTTTGACTACTGGGGCCAGGGAACC | | | | | | | RGAFDYWGQGTLVT | | | | | | |
| | | CTGGTCACCGTCTCCTCA | | | | | | | VSS | | | | | | |
| ADI-81669 | 6831 | GAGGTGCAGCTGTTGGAGTCTGGGGGAGGCGTGGTCCAGCC | | | | | | 7123 | EVQLLESGGGVVQP | | 7474 | 15 | | 15 | |
| | | TGGGAGGTCCCTGAGACTCTCCTGTGCAACCTCTGGATTCC | | | | | | | GRSLRLSCATSGFP | | | | | | |
| | | CCTTCAATACCCATGGCTTGAACTGGGTCCGCCTGGCTCCA | | | | | | | FNTHGLNWVRLAPG | | | | | | |
| | | GGCAAGGGGCTGCAGTGGGTGGCAGTTATATCCTATGATGG | | | | | | | KGLQWVAVISYDGN | | | | | | |
| | | AAATTATAAATACTATGCGGACTCCGTGAAGGGCCGATTCA | | | | | | | YKYYADSVKGRFTI | | | | | | |
| | | CCATCGCCAGAGACAATTCCAAGAACACGATGTATCTACAA | | | | | | | ARDNSKNTMYLQMN | | | | | | |
| | | ATGAACAGCCTGAGACCTGACGACACGGCTGTGTATTACTG | | | | | | | SLRPDDTAVYYCGR | | | | | | |
| | | TGGGAGAGCACGATCGACTTTTTGTACTAGTTCCAGCTGCT | | | | | | | ARSTFCTSSSCYSA | | | | | | |
| | | ACTCTGCTGCTTTTGATTTCGGGGGCCAAGGGACCACGGTC | | | | | | | AFDFGGQGTTVTVS | | | | | | |
| | | ACCGTCTCCTCA | | | | | | | S | | | | | | |
| ADI-81787 | 1286 | GAGGTGCAGCTGGTGCAGTCTGGGGGAGGCGTGGTCCAGCC | | | | | | 7124 | EVQLVQSGGGVVQP | | 7475 | 15 | | 12 | |
| | | TGGGAGGTCCCTGAGACTCTCCTGTGCAGCCTCTGGATTTA | | | | | | | GRSLRLSCAASGFT | | | | | | |
| | | CGTTCAGTAGTTATGCTATGCACTGGGTCCGCCAGACTCCA | | | | | | | FSSYAMHWVRQTPD | | | | | | |
| | | GACAAGGGGCTGGAGTGGGTGGCGGTTATACTAGATGATGG | | | | | | | KGLEWVAVILDDGR | | | | | | |
| | | AAGAGGTAAATTCTACGCAGACTCCGTGAAGGGCCGATTCA | | | | | | | GKFYADSVKGRFTI | | | | | | |
| | | CCATCTCCAGAGACAATCTCAAGAACACACTATATCTGCAA | | | | | | | SRDNLKNTLYLQMN | | | | | | |
| | | ATGAACACCGTGGGAGTTGAGGACACGGCTGTGTATTACTG | | | | | | | TVGVEDTAVYYCAR | | | | | | |
| | | TGCGAGAGACTCCAGCACCGCGGACTCTCTAGTTAGCTGGT | | | | | | | DSSTADSLVSWFDP | | | | | | |
| | | TCGACCCCTGGGGCCAGGGAACCCTGGTCACCGTCTCCTCA | | | | | | | WGQGTLVTVSS | | | | | | |
| ADI-81788 | 1287 | TTAGGTGCAGTTGGGGAGTCGGGCCCAGGGCTGGTGAAGCC | | | | | | 7125 | LGAVGESGPGLVKP | | 7476 | 15 | | 11 | |
| | | TTCACAGACCCTGTCCCTCACTTGCACTGTGTCTGGTCATT | | | | | | | SQTLSLTCTVSGHS | | | | | | |
| | | CCATTATCAGTGGTTCTTACTACAGTAGTTGGATCCGCCAG | | | | | | | IISGSYYSSWIRQL | | | | | | |
| | | CTCCCAGGAAAGGGCCTGGAGTGGATCGGGTACATGTATTA | | | | | | | PGKGLEWIGYMYYS | | | | | | |
| | | CAGTGGTAGCACGTATTACAACCCGTCCCTCAAGAGTCGAC | | | | | | | GSTYYNPSLKSRLT | | | | | | |
| | | TTACCATATCAATAGACACGTTTAAGAACCAGTACTCCCTG | | | | | | | ISIDTFKNQYSLKL | | | | | | |
| | | AAGCTGAGCTCTGTTACTGCCGCGGACACGGCGGTTTATTA | | | | | | | SSVTAADTAVYYCV | | | | | | |
| | | CTGTGTGAGAGAGTCTATTGTAGTAGCACCAGCAGCCGGGA | | | | | | | RESIVVAPAAGTYP | | | | | | |
| | | CGTACCCCTCCTACTACGGTATTGACGTCTGGGGCCAAGGG | | | | | | | SYYGIDVWGQGTTV | | | | | | |
| | | ACCACGGTCACCGTCTCCTCA | | | | | | | TVSS | | | | | | |
| ADI-81566 | 1286 | CAGGTCCAGCTGGTGCAGTCTGGGGCTGAGGTGAAGAAGCC | | | | | | 7126 | QVQLVQSGAEVKKP | | 7477 | 14 | | 10 | |
| | | TGGGTCCTCGGTGAAGGTCTCCTGCAAGGCTTCTGGAGACA | | | | | | | GSSVKVSCKASGDT | | | | | | |
| | | CCTTCAGTAATTATGCTATTACCTGGGTGCGACAGGCCCCT | | | | | | | FSNYAITWVRQAPG | | | | | | |
| | | GGACAAGGACTTGAGTGGATGGGAAGGATCATCCCTATTTT | | | | | | | QGLEWMGRIIPIFG | | | | | | |
| | | TGGTATAACAAATATCGCACAGAAGTTCCAGGGCAGAGTCA | | | | | | | ITNIAQKFQGRVTI | | | | | | |
| | | CCATTAGCGCGGACAAATCCACGGGAACAACCTACATGGAA | | | | | | | SADKSTGTTYMELS | | | | | | |
| | | CTGAGCAGCCTCAGATCTGAGGACACGGCCGTGTATTTTTG | | | | | | | SLRSEDTAVYFCAR | | | | | | |
| | | TGCGAGAGGTAGAACCGACGACTACCTTGACTCCTGGGGCC | | | | | | | GRTDDYLDSWGQGT | | | | | | |
| | | AGGGAACCCTGGTCACCGTCTCCTCA | | | | | | | LVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81662 | 34 | IGHV3- | EVQLV | 5332 | IIVSRN | 5734 | WVRQTP | 448 | VLYSGG | 607 | RFTISR | 6433 | ARDFHEG | 6749 | WGQGTT |
| | | 66 | ESGGG | | YMN | | GKGLEW | | TTFYAD | | DTSKNT | | AFDI | | VTVSS |
| | | | LVQPG | | | | VS | | SVKG | | LYLQMN | | | | |
| | | | GSLRL | | | | | | | | SLRLED | | | | |
| | | | SCAAS | | | | | | | | TAVYYC | | | | |
| | | | G | | | | | | | | | | | | |
| ADI-81694 | 64 | IGHV4- | PVQLL | 5492 | GSIGTS | 5735 | WIRQPP | 5857 | NIFKTG | 6127 | RVTISV | 6434 | ARAYTHS | 6750 | WGKGTT |
| | | 59 | ESGPG | | YWS | | GNGLEW | | RTNYNP | | DTSKKQ | | DFWSGYS | | VTVSS |
| | | | LVKPS | | | | IG | | ISLKS | | FSLRLS | | DYYDYYM | | |
| | | | ETLSL | | | | | | | | SVTAAD | | DV | | |
| | | | TCTVS | | | | | | | | TAVYFC | | | | |
| | | | G | | | | | | | | | | | | |
| ADI-81674 | 45 | IGHV4- | EVQLL | 5335 | DSISGY | 5707 | WLRQPP | 5858 | YIYDNG | 6128 | RVTISL | 6435 | ARGLTTG | 6751 | WGQGTT |
| | | 59 | ESGPG | | YWS | | GKGLEW | | NTNYNP | | DTSKNQ | | DAFDL | | VTVSS |
| | | | LVKPS | | | | VG | | SLKS | | FSLILS | | | | |
| | | | ETLSL | | | | | | | | AVTAAD | | | | |
| | | | TCTVS | | | | | | | | TAVFYC | | | | |
| | | | G | | | | | | | | | | | | |
| ADI-81675 | 45 | IGHV4- | EVQLL | 5335 | DSISGY | 5707 | WLRQPP | 5858 | YIYDNG | 6128 | RVTISL | 6435 | ARGLTTG | 6751 | WGQGTM |
| | | 59 | ESGPG | | YWS | | GKGLEW | | NTNYNP | | DTSKNQ | | DAFDL | | VTVSS |
| | | | LVKPS | | | | VG | | SLKS | | FSLILS | | | | |
| | | | ETLSL | | | | | | | | AVTAAD | | | | |
| | | | TCTVS | | | | | | | | TAVFYC | | | | |
| | | | G | | | | | | | | | | | | |
| ADI-81878 | 55 | IGHV3- | EVQLL | 5355 | FTFTNC | 5736 | WVRQAP | 437 | TIGDSG | 6129 | RFTISR | 6436 | AKNLDTP | 6752 | WGQGTT |
| | | 23 | ESGGG | | AMG | | GKGLEW | | RSTYYA | | DNSKNT | | NYDADDY | | VTVSS |
| | | | LVQPG | | | | VS | | DSVKG | | LHLRMN | | YYYYGMD | | |
| | | | GSLRL | | | | | | | | SLRADD | | V | | |
| | | | SCAAS | | | | | | | | TAAYYC | | | | |
| | | | G | | | | | | | | | | | | |
| ADI-81619 | 76 | IGHV5- | QVQLV | 5493 | YSFTSH | 5737 | WVRQMP | 476 | AVFPGD | 6130 | QVTISA | 6437 | ARLGYSY | 6753 | WGQGTL |
| | | 51 | QSGAE | | WIG | | GKGLEW | | SDTRYS | | DKSIST | | DTGDY | | VTVSS |
| | | | VKEPG | | | | MG | | PSFQG | | AYLHWS | | | | |
| | | | ESLRI | | | | | | | | GLKTSD | | | | |
| | | | SCKGS | | | | | | | | TAMYYC | | | | |
| | | | G | | | | | | | | | | | | |
| ADI-81744 | 20 | IGHV1- | EVQLL | 5494 | YTFTNH | 5738 | WVRQAP | 433 | IINPDG | 6131 | RVTMTS | 6438 | ARDFAFI | 6754 | WGQGTL |
| | | 46 | ESGAE | | YMH | | GQGLEW | | GSRTYA | | DTSTST | | PAAGPFE | | VTVSS |
| | | | VKKPG | | | | MG | | QKFQG | | VYMELS | | Y | | |
| | | | ASVKV | | | | | | | | SVRYED | | | | |
| | | | SCKTS | | | | | | | | TAVYYC | | | | |
| | | | G | | | | | | | | | | | | |
| ADI-81633 | 6 | IGHV3- | EVQLV | 5332 | FTFSAY | 5739 | WVRQAP | 5859 | NIKQDG | 606 | RFTISR | 6439 | ARVFWLR | 6755 | WGQGTL |
| | | 7 | ESGGG | | WMT | | GKGLEW | | SEKYYV | | DNAKNA | | GGVDF | | VTVSS |
| | | | LVQPG | | | | VP | | DSVKG | | LYLQIN | | | | |
| | | | GSLRL | | | | | | | | SLRAED | | | | |
| | | | SCAAS | | | | | | | | TAVYTC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81662 | 1287 | GAGGTGCAGCTGGTGGAGTCTGGGGGAGGCTTGGTCCAGCC | | | | | | 7127 | EVQLVESGGGLVQP | | 7478 | 13 | | 10 | |
| | | GGGGGGGTCCCTGAGACTCTCCTGTGCAGCCTCTGGAATCA | | | | | | | GGSLRLSCAASGII | | | | | | |
| | | TCGTCAGTAGGAACTACATGAACTGGGTCCGCCAGACTCCG | | | | | | | VSRNYMNWVRQTPG | | | | | | |
| | | GGGAAGGGGCTGGAGTGGGTCTCAGTCCTTTATAGCGGTGG | | | | | | | KGLEWVSVLYSGGT | | | | | | |
| | | GACAACATTCTACGCAGACTCCGTGAAGGGCCGATTCACCA | | | | | | | TFYADSVKGRFTIS | | | | | | |
| | | TCTCCAGAGACACTTCCAAGAACACTCTGTATCTTCAAATG | | | | | | | RDTSKNTLYLQMNS | | | | | | |
| | | AACAGCCTGAGACTTGAGGACACGGCTGTCTATTACTGTGC | | | | | | | LRLEDTAVYYCARD | | | | | | |
| | | GAGAGATTTCCACGAAGGTGCTTTTGATATCTGGGGCCAAG | | | | | | | FHEGAFDIWGQGTT | | | | | | |
| | | GGACCACGGTCACCGTCTCCTCA | | | | | | | VTVSS | | | | | | |
| ADI-81694 | 1291 | CCCGTGCAGCTGTTGGAGTCGGGCCCAGGACTGGTGAAGCC | | | | | | 7128 | PVQLLESGPGLVKP | | 7479 | 13 | | 12 | |
| | | TTCGGAGACCCTGTCCCTCACCTGCACTGTCTCTGGTGGCT | | | | | | | SETLSLTCTVSGGS | | | | | | |
| | | CCATCGGTACTTCCTACTGGAGCTGGATCCGGCAGCCCCCA | | | | | | | IGTSYWSWIRQPPG | | | | | | |
| | | GGGAACGGACTGGAGTGGATTGGGAATATCTTTAAAACTGG | | | | | | | NGLEWIGNIFKTGR | | | | | | |
| | | AAGAACCAACTACAACCCCTCCCTCAAGAGTCGCGTCACCA | | | | | | | TNYNPSLKSRVTIS | | | | | | |
| | | TATCAGTAGACACGTCCAAGAAACAATTCTCCCTGAGGCTG | | | | | | | VDTSKKQFSLRLSS | | | | | | |
| | | AGCTCTGTGACCGCTGCGGACACGGCCGTGTATTTCTGTGC | | | | | | | VTAADTAVYFCARA | | | | | | |
| | | GAGGGCTTATACGCATTCCGATTTTTGGAGTGGATATTCTG | | | | | | | YTHSDFWSGYSDYY | | | | | | |
| | | ACTACTACGACTACTACATGGACGTCTGGGGCAAAGGGACC | | | | | | | DYYMDVWGKGTTVT | | | | | | |
| | | ACGGTCACCGTCTCCTCA | | | | | | | VSS | | | | | | |
| ADI-81674 | 1287 | GAGGTGCAGCTGTTGGAGTCGGGCCCAGGACTGGTGAAGCC | | | | | | 7129 | EVQLLESGPGLVKP | | 7480 | 12 | | 11 | |
| | | TTCGGAGACCCTGTCCCTCACCTGCACTGTCTCTGGTGACT | | | | | | | SETLSLTCTVSGDS | | | | | | |
| | | CCATCAGTGGTTACTACTGGAGCTGGCTCCGGCAGCCCCCA | | | | | | | ISGYYWSWLRQPPG | | | | | | |
| | | GGGAAGGGACTGGAGTGGGTTGGCTATATCTATGACAATGG | | | | | | | KGLEWVGYIYDNGN | | | | | | |
| | | GAACACCAACTACAACCCCTCCCTCAAGAGTCGAGTCACCA | | | | | | | TNYNPSLKSRVTIS | | | | | | |
| | | TATCACTAGACACGTCCAAGAACCAGTTCTCCCTGATACTG | | | | | | | LDTSKNQFSLILSA | | | | | | |
| | | AGCGCTGTGACCGCTGCGGACACGGCCGTCTTTTACTGTGC | | | | | | | VTAADTAVFYCARG | | | | | | |
| | | GCGAGGACTAACTACGGGTGATGCTTTTGATCTCTGGGGCC | | | | | | | LTTGDAFDLWGQGT | | | | | | |
| | | AAGGGACCACGGTCACCGTCTCCTCA | | | | | | | TVTVSS | | | | | | |
| ADI-81675 | 1299 | GAGGTGCAGCTGTTGGAGTCGGGCCCAGGACTGGTGAAGCC | | | | | | 7130 | EVQLLESGPGLVKP | | 7481 | 12 | | 11 | |
| | | TTCGGAGACCCTGTCCCTCACCTGCACTGTCTCTGGTGACT | | | | | | | SETLSLTCTVSGDS | | | | | | |
| | | CCATCAGTGGTTACTACTGGAGCTGGCTCCGGCAGCCCCCA | | | | | | | ISGYYWSWLRQPPG | | | | | | |
| | | GGGAAGGGACTGGAGTGGGTTGGCTATATCTATGACAATGG | | | | | | | KGLEWVGYIYDNGN | | | | | | |
| | | GAACACCAACTACAACCCCTCCCTCAAGAGTCGAGTCACCA | | | | | | | TNYNPSLKSRVTIS | | | | | | |
| | | TATCACTAGACACGTCCAAGAACCAGTTCTCCCTGATACTG | | | | | | | LDTSKNQFSLILSA | | | | | | |
| | | AGCGCTGTGACCGCTGCGGACACGGCCGTCTTTTACTGTGC | | | | | | | VTAADTAVFYCARG | | | | | | |
| | | GCGAGGACTAACTACGGGTGATGCTTTTGATCTCTGGGGCC | | | | | | | LTTGDAFDLWGQGT | | | | | | |
| | | AAGGGACAATGGTCACCGTCTCTTCA | | | | | | | MVTVSS | | | | | | |
| ADI-81878 | 1287 | GAGGTGCAGCTGTTGGAGTCTGGGGGAGGCTTGGTACAGCC | | | | | | 7131 | EVQLLESGGGLVQP | | 7482 | 12 | | 12 | |
| | | TGGGGGGTCCCTGAGACTCTCCTGTGCAGCCTCTGGATTCA | | | | | | | GGSLRLSCAASGFT | | | | | | |
| | | CCTTTACCAACTGTGCCATGGGCTGGGTCCGCCAGGCTCCA | | | | | | | FTNCAMGWVRQAPG | | | | | | |
| | | GGGAAGGGGCTGGAGTGGGTCTCAACTATTGGTGATAGTGG | | | | | | | KGLEWVSTIGDSGR | | | | | | |
| | | TCGTAGCACATACTACGCAGACTCCGTGAAGGGCCGGTTCA | | | | | | | STYYADSVKGRFTI | | | | | | |
| | | CCATCTCCAGAGACAATTCCAAGAACACGCTGCATCTGCGA | | | | | | | SRDNSKNTLHLRMN | | | | | | |
| | | ATGAACAGCCTGAGAGCCGACGACACGGCCGCATATTACTG | | | | | | | SLRADDTAAYYCAK | | | | | | |
| | | TGCGAAAAACCTCGACACCCCGAACTACGACGCCGACGATT | | | | | | | NLDTPNYDADDYYY | | | | | | |
| | | ACTACTACTACTACGGTATGGACGTCTGGGGCCAAGGGACC | | | | | | | YYGMDVWGQGTTVT | | | | | | |
| | | ACGGTCACCGTCTCCTCA | | | | | | | VSS | | | | | | |
| ADI-81619 | 1286 | CAGGTCCAGCTGGTGCAGTCTGGAGCAGAGGTGAAAGAGCC | | | | | | 7132 | QVQLVQSGAEVKEP | | 7483 | 10 | | 9 | |
| | | GGGGGAGTCTCTGAGGATCTCCTGTAAGGGTTCTGGATACA | | | | | | | GESLRISCKGSGYS | | | | | | |
| | | GCTTTACCAGCCACTGGATCGGCTGGGTGCGCCAGATGCCC | | | | | | | FTSHWIGWVRQMPG | | | | | | |
| | | GGGAAAGGCCTGGAGTGGATGGGGGCCGTCTTTCCTGGTGA | | | | | | | KGLEWMGAVFPGDS | | | | | | |
| | | CTCTGATACCAGATACAGCCCGTCCTTCCAAGGCCAGGTCA | | | | | | | DTRYSPSFQGQVTI | | | | | | |
| | | CCATCTCAGCCGACAAGTCCATCAGCACCGCCTACCTACAC | | | | | | | SADKSISTAYLHWS | | | | | | |
| | | TGGAGCGGCCTGAAGACCTCGGACACCGCCATGTATTACTG | | | | | | | GLKTSDTAMYYCAR | | | | | | |
| | | TGCGAGACTGGGGTACAGCTATGACACGGGTGACTACTGGG | | | | | | | LGYSYDTGDYWGQG | | | | | | |
| | | GCCAGGGAACCCTGGTCACCGTCTCCTCA | | | | | | | TLVTVSS | | | | | | |
| ADI-81744 | 1286 | GAGGTGCAGCTGTTGGAGTCTGGGGCTGAGGTGAAGAAGCC | | | | | | 7133 | EVQLLESGAEVKKP | | 7484 | 10 | | 9 | |
| | | TGGGGCCTCAGTGAAGGTTTCCTGCAAGACATCTGGATACA | | | | | | | GASVKVSCKTSGYT | | | | | | |
| | | CCTTCACCAACCACTATATGCACTGGGTGCGCCAGGCCCCT | | | | | | | FTNHYMHWVRQAPG | | | | | | |
| | | GGACAAGGGCTTGAGTGGATGGGAATAATCAACCCTGATGG | | | | | | | QGLEWMGIINPDGG | | | | | | |
| | | TGGTAGCAGAACCTACGCACAGAAGTTCCAGGGCAGAGTCA | | | | | | | SRTYAQKFQGRVTM | | | | | | |
| | | CCATGACCAGCGACACGTCCACGAGCACAGTGTACATGGAG | | | | | | | TSDTSTSTVYMELS | | | | | | |
| | | TTGAGCAGCGTGAGATATGAGGACACGGCCGTGTATTACTG | | | | | | | SVRYEDTAVYYCAR | | | | | | |
| | | TGCGAGAGATTTTGCCTTTATACCAGCAGCTGGTCCGTTTG | | | | | | | DFAFIPAAGPFEYW | | | | | | |
| | | AATATTGGGGCCAGGGAACCCTGGTCACCGTCTCCTCA | | | | | | | GQGTLVTVSS | | | | | | |
| ADI-81633 | 1286 | GAGGTGCAGCTGGTGGAGTCTGGGGGAGGCTTGGTCCAGCC | | | | | | 7134 | EVQLVESGGGLVQP | | 7485 | 9 | | 6 | |
| | | TGGAGGGTCCCTGAGACTCTCTTGTGCAGCCTCTGGATTCA | | | | | | | GGSLRLSCAASGFT | | | | | | |
| | | CCTTTAGTGCCTATTGGATGACCTGGGTCCGCCAGGCTCCA | | | | | | | FSAYWMTWVRQAPG | | | | | | |
| | | GGGAAGGGGCTGGAGTGGGTGCCCAACATAAAGCAAGATGG | | | | | | | KGLEWVPNIKQDGS | | | | | | |
| | | AAGTGAGAAATACTATGTGGACTCTGTGAAGGGCCGATTCA | | | | | | | EKYYVDSVKGRFTI | | | | | | |
| | | CCATCTCCAGAGACAACGCCAAGAACGCACTGTATCTGCAA | | | | | | | SRDNAKNALYLQIN | | | | | | |
| | | ATCAACAGCCTGAGAGCCGAGGACACGGCCGTGTATACTTG | | | | | | | SLRAEDTAVYTCAR | | | | | | |
| | | TGCGCGAGTTTTCTGGCTGCGCGGCGGGGTTGACTTCTGGG | | | | | | | VFWLRGGVDFWGQG | | | | | | |
| | | GCCAGGGAACCCTGGTCACCGTCTCCTCA | | | | | | | TLVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81700 | 70 | IGHV3- | EVQLV | 5495 | IIVSRN | 5740 | WVRQAP | 437 | VIFPGG | 6132 | RFTISR | 876 | AREDNNN | 1123 | WGQGTT |
| | | 53 | ETGGG | | YMS | | GKGLEW | | STYYAD | | DDSKNT | | AFDI | | VTVSS |
| | | | LIQPG | | | | VS | | SVKG | | LYLQMN | | | | |
| | | | ESLRL | | | | | | | | SLRAED | | | | |
| | | | SCAAS | | | | | | | | TAVYYC | | | | |
| | | | E | | | | | | | | | | | | |
| ADI-81797 | 73 | IGHV3- | QVQLL | 74 | FTFSNY | 5741 | WVRQSP | 5860 | AISGSG | 6133 | RFTISR | 6440 | AKQSVNR | 6756 | WGQGTL |
| | | 23 | ESGGG | | AMS | | GKGLEW | | DDTYYA | | DNSKNT | | NPGWDFL | | VTVSS |
| | | | LVQPG | | | | VS | | DSVKG | | LYLQMS | | VSTTEDY | | |
| | | | GSLRL | | | | | | | | LLRAED | | FDS | | |
| | | | SCAAS | | | | | | | | TAVYFC | | | | |
| | | | G | | | | | | | | | | | | |
| ADI-81854 | 32 | IGHV1- | EVQLV | 5385 | YTFSSF | 5742 | WVRQAP | 433 | WISTYE | 6134 | RVTMTT | 993 | ARVFSYY | 6757 | WGQGTL |
| | | 18 | ESGAE | | GVS | | GQGLEW | | GNTNYA | | DTSTST | | DSSGWGT | | VTVSS |
| | | | VKKPG | | | | MG | | QNFQG | | AYMELR | | AYYFDS | | |
| | | | ASVKV | | | | | | | | SLRSDD | | | | |
| | | | SCKAS | | | | | | | | TAVYYC | | | | |
| | | | G | | | | | | | | | | | | |
| ADI-81866 | 44 | IGHV1- | EVQLL | 5496 | GSFSNY | 5743 | WVRQAP | 433 | GIIPMF | 6135 | RVRIIA | 6441 | ARDEATI | 6758 | WGQGTT |
| | | 69 | QSGAE | | GIS | | GQGLEW | | GAAKYA | | DESTST | | TVVQGVA | | VTVSS |
| | | | VKKPG | | | | MG | | QKFQG | | AYMELS | | TRHGYDI | | |
| | | | SSVKV | | | | | | | | SLRSED | | | | |
| | | | SCKAS | | | | | | | | TAVYYC | | | | |
| | | | G | | | | | | | | | | | | |
| ADI-81903 | 77 | IGHV3- | EVQLL | 5324 | FTFDDY | 214 | WVRQAP | 437 | GITWNS | 6136 | RFTISR | 6442 | AKDTGSK | 6759 | WGQGTT |
| | | 9 | ESGGG | | AMH | | GKGLEW | | GTIGYA | | DNSKNS | | DYYYYGM | | VTVSS |
| | | | LVQPG | | | | VS | | DSAKG | | LYLHIS | | DV | | |
| | | | RSLRL | | | | | | | | SLRTED | | | | |
| | | | SCAAS | | | | | | | | TALYYC | | | | |
| | | | G | | | | | | | | | | | | |
| ADI-81703 | 73 | IGHV1- | QVQLV | 5497 | YRFNIY | 5744 | WVRQAP | 433 | WSSAYN | 6137 | RVTMTT | 6443 | ARALGEG | 6760 | WGQGTL |
| | | 18 | ESGAE | | GIT | | GQGLEW | | GNTNYA | | DTSTST | | SGSVFGY | | VTVSS |
| | | | VKKPG | | | | MG | | QKLQG | | AYMELR | | | | |
| | | | ASVKV | | | | | | | | SLTSDD | | | | |
| | | | SCKAS | | | | | | | | TAVYYC | | | | |
| | | | G | | | | | | | | | | | | |
| ADI-81739 | 16 | IGHV3- | EVQLL | 5324 | FTFDDY | 214 | WVRQAP | 437 | GISWNG | 6138 | RFIISR | 6444 | AKSSSVG | 6761 | WGQGAL |
| | | 9 | ESGGG | | AMH | | GKGLEW | | GIINYA | | DNAKNS | | DEGEFSS | | VTVSS |
| | | | LVQPG | | | | VS | | DSVKG | | LYLQMI | | FDY | | |
| | | | RSLRL | | | | | | | | RLRAED | | | | |
| | | | SCAAS | | | | | | | | TALYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81700 | 1287 | GAGGTGCAGCTGGTGGAGACTGGAGGAGGCTTGATCCAGCC | | | | | | 7135 | EVQLVETGGGLIQP | | 7486 | 9 | | 8 | |
| | | GGGGGAGTCCCTGAGACTCTCCTGTGCAGCCTCTGAGATCA | | | | | | | GESLRLSCAASEII | | | | | | |
| | | TCGTCAGTAGGAACTACATGAGCTGGGTCCGCCAGGCTCCA | | | | | | | VSRNYMSWVRQAPG | | | | | | |
| | | GGGAAGGGGCTGGAGTGGGTCTCAGTTATTTTTCCCGGTGG | | | | | | | KGLEWVSVIFPGGS | | | | | | |
| | | GAGCACTTACTACGCAGACTCCGTGAAGGGCCGATTCACCA | | | | | | | TYYADSVKGRFTIS | | | | | | |
| | | TCTCCCGAGACGATTCCAAGAACACGCTGTATCTTCAAATG | | | | | | | RDDSKNTLYLQMNS | | | | | | |
| | | AACAGCCTGAGAGCCGAGGACACGGCCGTCTATTACTGTGC | | | | | | | LRAEDTAVYYCARE | | | | | | |
| | | GAGAGAAGACAATAATAATGCTTTTGATATTTGGGGGCAAG | | | | | | | DNNNAFDIWGQGTT | | | | | | |
| | | GGACCACGGTCACCGTCTCCTCA | | | | | | | VTVSS | | | | | | |
| ADI-81797 | 1286 | CAGGTGCAGCTGTTGGAGTCTGGGGGAGGCTTGGTACAGCC | | | | | | 7136 | QVQLLESGGGLVQP | | 7487 | 9 | | 7 | |
| | | TGGGGGGTCCCTGAGACTCTCCTGTGCAGCCTCTGGATTCA | | | | | | | GGSLRLSCAASGFT | | | | | | |
| | | CCTTTAGCAACTATGCCATGAGTTGGGTCCGCCAGTCTCCA | | | | | | | FSNYAMSWVRQSPG | | | | | | |
| | | GGGAAGGGGCTGGAGTGGGTCTCAGCTATTAGTGGTAGTGG | | | | | | | KGLEWVSAISGSGD | | | | | | |
| | | TGATGACACATACTACGCAGACTCCGTGAAGGGCCGGTTCA | | | | | | | DTYYADSVKGRFTI | | | | | | |
| | | CCATCTCCAGAGACAATTCCAAGAACACGCTGTATCTGCAA | | | | | | | SRDNSKNTLYLQMS | | | | | | |
| | | ATGAGCCTCCTGAGAGCCGAGGACACGGCCGTATATTTCTG | | | | | | | LLRAEDTAVYFCAK | | | | | | |
| | | TGCGAAACAGTCGGTTAACCGAAACCCAGGATGGGACTTCC | | | | | | | QSVNRNPGWDFLVS | | | | | | |
| | | TAGTGTCTACCACTGAGGACTACTTTGACTCTTGGGGCCAG | | | | | | | TTEDYFDSWGQGTL | | | | | | |
| | | GGAACCCTGGTCACCGTCTCCTCA | | | | | | | VTVSS | | | | | | |
| ADI-81854 | 1286 | GAGGTGCAGCTGGTGGAGTCTGGAGCTGAGGTGAAGAAGCC | | | | | | 7137 | EVQLVESGAEVKKP | | 7488 | 8 | | 7 | |
| | | TGGGGCCTCAGTGAAGGTCTCCTGCAAGGCTTCTGGTTACA | | | | | | | GASVKVSCKASGYT | | | | | | |
| | | CCTTTTCCAGTTTTGGTGTCAGCTGGGTGCGACAGGCCCCT | | | | | | | FSSFGVSWVRQAPG | | | | | | |
| | | GGACAAGGGCTTGAGTGGATGGGGTGGATCAGTACTTACGA | | | | | | | QGLEWMGWISTYEG | | | | | | |
| | | AGGTAACACAAACTATGCACAGAACTTCCAGGGCAGAGTCA | | | | | | | NTNYAQNFQGRVTM | | | | | | |
| | | CCATGACCACGGACACATCCACGAGCACAGCCTACATGGAG | | | | | | | TTDTSTSTAYMELR | | | | | | |
| | | CTGAGGAGCCTGAGATCTGACGACACGGCCGTCTATTACTG | | | | | | | SLRSDDTAVYYCAR | | | | | | |
| | | TGCGAGAGTCTTCAGTTACTATGATAGTAGTGGTTGGGGGA | | | | | | | VFSYYDSSGWGTAY | | | | | | |
| | | CTGCTTACTACTTTGACTCCTGGGGCCAGGGAACCCTGGTC | | | | | | | YFDSWGQGTLVTVS | | | | | | |
| | | ACCGTCTCCTCA | | | | | | | S | | | | | | |
| ADI-81866 | 1287 | GAGGTGCAGCTGTTGCAGTCTGGGGCTGAGGTGAAGAAGCC | | | | | | 7138 | EVQLLQSGAEVKKP | | 7489 | 8 | | 8 | |
| | | TGGGTCCTCGGTGAAGGTCTCCTGCAAGGCTTCTGGAGGCA | | | | | | | GSSVKVSCKASGGS | | | | | | |
| | | GCTTCAGTAACTATGGTATCAGCTGGGTGCGACAGGCCCCT | | | | | | | FSNYGISWVRQAPG | | | | | | |
| | | GGACAAGGGCTTGAGTGGATGGGAGGGATCATCCCTATGTT | | | | | | | QGLEWMGGIIPMFG | | | | | | |
| | | TGGTGCAGCAAAATACGCACAGAAGTTCCAGGGCAGAGTCA | | | | | | | AAKYAQKFQGRVRI | | | | | | |
| | | GGATTATCGCGGACGAATCCACGAGCACAGCCTATATGGAG | | | | | | | IADESTSTAYMELS | | | | | | |
| | | CTGAGCAGCCTGAGATCTGAGGACACGGCCGTATATTACTG | | | | | | | SLRSEDTAVYYCAR | | | | | | |
| | | TGCGAGAGACGAAGCTACGATAACAGTGGTTCAGGGGGTCG | | | | | | | DEATITVVQGVATR | | | | | | |
| | | CCACCCGTCATGGTTATGATATCTGGGGCCAAGGGACCACG | | | | | | | HGYDIWGQGTTVTV | | | | | | |
| | | GTCACCGTCTCCTCA | | | | | | | SS | | | | | | |
| ADI-81903 | 1287 | GAGGTGCAGCTGTTGGAGTCTGGGGGAGGCTTGGTACAGCC | | | | | | 7139 | EVQLLESGGGLVQP | | 7490 | 8 | | 8 | |
| | | TGGCAGGTCCCTGAGACTCTCCTGTGCAGCCTCTGGATTCA | | | | | | | GRSLRLSCAASGFT | | | | | | |
| | | CCTTCGATGATTATGCCATGCACTGGGTCCGGCAAGCTCCA | | | | | | | FDDYAMHWVRQAPG | | | | | | |
| | | GGGAAGGGCCTGGAGTGGGTCTCAGGTATTACTTGGAATAG | | | | | | | KGLEWVSGITWNSG | | | | | | |
| | | TGGTACCATAGGCTATGCGGACTCTGCGAAGGGCCGATTCA | | | | | | | TIGYADSAKGRFTI | | | | | | |
| | | CCATCTCCAGAGACAACTCCAAGAACTCCCTGTATCTGCAC | | | | | | | SRDNSKNSLYLHIS | | | | | | |
| | | ATCAGCAGTCTGAGAACTGAGGACACGGCCTTGTATTACTG | | | | | | | SLRTEDTALYYCAK | | | | | | |
| | | TGCAAAAGATACAGGATCCAAGGACTACTATTACTACGGTA | | | | | | | DTGSKDYYYYGMDV | | | | | | |
| | | TGGACGTCTGGGGCCAAGGGACCACGGTCACCGTCTCCTCA | | | | | | | WGQGTTVTVSS | | | | | | |
| ADI-81703 | 1286 | CAGGTGCAGCTGGTGGAGTCTGGAGCTGAGGTGAAGAAGCC | | | | | | 7140 | QVQLVESGAEVKKP | | 7491 | 7 | | 6 | |
| | | GGGGGCCTCAGTGAAGGTCTCTTGCAAGGCTTCTGGTTACA | | | | | | | GASVKVSCKASGYR | | | | | | |
| | | GGTTCAACATCTATGGTATCACCTGGGTGCGACAGGCCCCT | | | | | | | FNIYGITWVRQAPG | | | | | | |
| | | GGACAAGGGCTTGAGTGGATGGGATGGAGCAGCGCTTACAA | | | | | | | QGLEWMGWSSAYNG | | | | | | |
| | | TGGTAACACAAACTATGCACAGAAGCTCCAGGGCAGAGTCA | | | | | | | NTNYAQKLQGRVTM | | | | | | |
| | | CCATGACCACAGACACATCCACGAGTACAGCCTACATGGAG | | | | | | | TTDTSTSTAYMELR | | | | | | |
| | | TTGAGGAGCCTGACATCTGACGACACGGCCGTGTATTACTG | | | | | | | SLTSDDTAVYYCAR | | | | | | |
| | | TGCGAGAGCATTGGGGGAAGGTTCGGGGTCAGTCTTTGGGT | | | | | | | ALGEGSGSVFGYWG | | | | | | |
| | | ACTGGGGCCAGGGAACCCTGGTCACCGTCTCCTCA | | | | | | | QGTLVTVSS | | | | | | |
| ADI-81739 | 1285 | TGGCAGGTCCCTGAGACTCTCCTGTGCAGCCTCTGGATTCA | | | | | | 7141 | EVQLLESGGGLVQP | | 7492 | 7 | | 6 | |
| | | CCTTTGATGATTATGCCATGCACTGGGTCCGGCAAGCTCCA | | | | | | | GRSLRLSCAASGFT | | | | | | |
| | | GGGAAGGGCCTGGAGTGGGTCTCAGGTATTAGTTGGAATGG | | | | | | | FDDYAMHWVRQAPG | | | | | | |
| | | TGGTATCATTAACTATGCGGACTCTGTGAAGGGCCGATTCA | | | | | | | KGLEWVSGISWNGG | | | | | | |
| | | TCATCTCCAGAGACAACGCCAAGAACTCCCTGTATCTGCAA | | | | | | | IINYADSVKGRFII | | | | | | |
| | | ATGATCCGTCTGAGAGCTGAGGACACGGCCTTGTATTACTG | | | | | | | SRDNAKNSLYLQMI | | | | | | |
| | | TGCAAAATCCAGTTCCGTCGGGGACGAGGGGGAGTTCTCGT | | | | | | | RLRAEDTALYYCAK | | | | | | |
| | | CTTTTGACTACTGGGGCCAGGGAGCCCTGGTCACCGTCTCC | | | | | | | SSSVGDEGEFSSFD | | | | | | |
| | | TCA | | | | | | | YWGQGALVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81837 | 16 | IGHV3- | QVQLV | 24 | FTFSSY | 5603 | WVRQAP | 431 | GVSGSG | 6139 | RFTISR | 777 | GKDSNLI | 6762 | WGQGTL |
| | | 23 | ESGGG | | AMS | | GKGLEW | | DGRFYA | | DNSKNT | | TQIVVDE | | VTVSS |
| | | | LVQPG | | | | VA | | DSVKG | | LYLQMN | | Y | | |
| | | | GSLRL | | | | | | | | SLRAED | | | | |
| | | | SCAAS | | | | | | | | TAVYYC | | | | |
| | | | G | | | | | | | | | | | | |
| ADI-81907 | 81 | IGHV3- | EVQLV | 5498 | FTFSDY | 5745 | WVRQAP | 5861 | VIWYDG | 6140 | RFTISR | 777 | AANGYTP | 6763 | WGHGTL |
| | | 33 | ESGGG | | NMH | | GKGPEW | | SDKYYA | | DNSKNT | | GGDFFDP | | VTVSS |
| | | | VVQLG | | | | VA | | DSVKG | | LYLQMN | | | | |
| | | | RSLRL | | | | | | | | SLRAED | | | | |
| | | | SCAAS | | | | | | | | TAVYYC | | | | |
| | | | G | | | | | | | | | | | | |
| ADI-81654 | 27 | IGHV2- | QVQLK | 5499 | FSLSTY | 5746 | WIRQPP | 441 | LIYYDD | 6141 | RLTITK | 6445 | ARHTIAT | 6764 | WGQGTL |
| | | 5 | QSGPT | | GVGVG | | GKALEW | | DKRYSP | | DTSQNQ | | IFDFDS | | VTVSS |
| | | | LVKPT | | | | LA | | SLKS | | VVLTMT | | | | |
| | | | QTLTL | | | | | | | | NMDPVD | | | | |
| | | | TCTFS | | | | | | | | TATYYC | | | | |
| | | | G | | | | | | | | | | | | |
| ADI-81677 | 47 | IGHV3- | EVQLV | 5500 | FTFSTY | 5747 | WVRQAP | 437 | SISSSN | 6142 | RFTISR | 6446 | AKDLTYY | 6765 | WGQGTT |
| | | 21 | ESGGG | | TMN | | GKGLEW | | NFIYYA | | DNAKNS | | YDSSDYY | | VTVSS |
| | | | LVKPG | | | | VS | | DSVKG | | VYLQMN | | GPNAFDI | | |
| | | | GSLRL | | | | | | | | SLRAED | | | | |
| | | | SCAAS | | | | | | | | TAVYYC | | | | |
| | | | G | | | | | | | | | | | | |
| ADI-81790 | 66 | IGHV3- | EVQLL | 5501 | FTFRSY | 5748 | WVRQAP | 431 | AISYDG | 6143 | RFTISR | 6447 | ARDPSGT | 6766 | WGQGTT |
| | | 30 | ESGGG | | AMH | | GKGLEW | | SNKYFA | | DHSKNT | | MVRGGIV | | VTVSS |
| | | | VVQPG | | | | VA | | DSVKG | | LYLQMN | | YYHYGMD | | |
| | | | TSLRL | | | | | | | | SLRAAD | | V | | |
| | | | SCAAS | | | | | | | | TAVYYC | | | | |
| | | | G | | | | | | | | | | | | |
| ADI-81755 | 31 | IGHV4- | EVQLQ | 5502 | GSFSGY | 206 | WIRQSP | 434 | EINHSG | 527 | RVTISV | 6448 | ARGKAIY | 6767 | WGQGTL |
| | | 34 | QWGAG | | YWS | | GKGLEW | | STNYNP | | DTSKSQ | | DFWRGYN | | VTVSS |
| | | | LLKPS | | | | IG | | SLKS | | FSLKLN | | GRALNFD | | |
| | | | ETLSR | | | | | | | | SVTDAD | | N | | |
| | | | TCAVY | | | | | | | | TAVYYC | | | | |
| | | | G | | | | | | | | | | | | |
| ADI-81893 | 69 | IGHV1- | QVQLL | 5448 | GSFSSD | 5749 | WVRQAP | 433 | GINPIF | 6144 | RVTITA | 1006 | AKDQVTS | 6768 | WGQGTT |
| | | 69 | ESGAE | | AIN | | GQGLEW | | GTANYA | | DESTST | | PLYGMDV | | VTVSS |
| | | | VKKPG | | | | MG | | QKFRG | | AYMELS | | | | |
| | | | SSVKV | | | | | | | | SLRSED | | | | |
| | | | SCKAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81837 | 1286 | CAGGTGCAGCTGGTGGAGTCTGGGGGAGGCTTGGTACAGCC | | | | | | 7142 | QVQLVESGGGLVQP | | 7493 | 7 | | 7 | |
| | | GGGGGGGTCCCTGAGACTCTCCTGTGCAGCCTCGGGATTCA | | | | | | | GGSLRLSCAASGFT | | | | | | |
| | | CCTTTAGCAGCTATGCCATGAGCTGGGTCCGCCAGGCTCCA | | | | | | | FSSYAMSWVRQAPG | | | | | | |
| | | GGGAAGGGGCTGGAGTGGGTCGCAGGTGTTAGTGGTAGTGG | | | | | | | KGLEWVAGVSGSGD | | | | | | |
| | | TGATGGCAGATTCTACGCAGACTCCGTGAAGGGCCGGTTCA | | | | | | | GRFYADSVKGRFTI | | | | | | |
| | | CCATCTCCAGAGACAATTCCAAGAACACGCTCTATCTGCAA | | | | | | | SRDNSKNTLYLQMN | | | | | | |
| | | ATGAACAGCCTGAGAGCCGAGGACACGGCCGTATATTACTG | | | | | | | SLRAEDTAVYYCGK | | | | | | |
| | | TGGGAAAGATAGTAACCTTATTACTCAAATAGTAGTTGATG | | | | | | | DSNLITQIVVDEYW | | | | | | |
| | | AGTACTGGGGCCAGGGAACCCTGGTCACCGTCTCCTCA | | | | | | | GQGTLVTVSS | | | | | | |
| ADI-81907 | 6804 | GAGGTCCAGCTGGTGGAGTCTGGGGGCGGCGTGGTCCAGCT | | | | | | 7143 | EVQLVESGGGVVQL | | 7494 | 7 | | 5 | |
| | | TGGGAGGTCCCTGAGACTCTCCTGCGCAGCGTCAGGATTCA | | | | | | | GRSLRLSCAASGFT | | | | | | |
| | | CCTTCAGTGACTATAACATGCACTGGGTCCGCCAGGCTCCA | | | | | | | FSDYNMHWVRQAPG | | | | | | |
| | | GGCAAGGGGCCGGAGTGGGTGGCAGTTATATGGTATGATGG | | | | | | | KGPEWVAVIWYDGS | | | | | | |
| | | AAGCGATAAATATTATGCAGACTCCGTGAAGGGCCGATTCA | | | | | | | DKYYADSVKGRFTI | | | | | | |
| | | CCATCTCCAGAGACAATTCCAAGAACACTCTGTATCTGCAA | | | | | | | SRDNSKNTLYLQMN | | | | | | |
| | | ATGAATAGCCTGAGAGCCGAGGACACGGCTGTGTATTACTG | | | | | | | SLRAEDTAVYYCAA | | | | | | |
| | | TGCGGCAAATGGCTACACTCCAGGTGGGGACTTCTTCGATC | | | | | | | NGYTPGGDFFDPWG | | | | | | |
| | | CTTGGGGCCACGGAACCCTGGTCACCGTCTCCTCA | | | | | | | HGTLVTVSS | | | | | | |
| ADI-81654 | 1286 | CAGGTCCAGCTGAAGCAGTCTGGTCCTACGCTGGTGAAACC | | | | | | 7144 | QVQLKQSGPTLVKP | | 7495 | 6 | | 3 | |
| | | CACACAGACCCTCACACTGACCTGCACCTTCTCTGGGTTCT | | | | | | | TQTLTLTCTFSGFS | | | | | | |
| | | CACTCAGCACTTATGGAGTGGGTGTGGGCTGGATCCGTCAG | | | | | | | LSTYGVGVGWIRQP | | | | | | |
| | | CCCCCAGGAAAGGCCCTGGAGTGGCTTGCACTCATTTATTA | | | | | | | PGKALEWLALIYYD | | | | | | |
| | | CGATGATGATAAGCGCTACAGCCCATCTCTGAAGAGCAGGC | | | | | | | DDKRYSPSLKSRLT | | | | | | |
| | | TCACCATCACCAAGGACACCTCCCAGAACCAGGTGGTCCTT | | | | | | | ITKDTSQNQVVLTM | | | | | | |
| | | ACAATGACCAACATGGACCCTGTGGACACAGCCACATATTA | | | | | | | TNMDPVDTATYYCA | | | | | | |
| | | CTGTGCCCGACACACTATAGCGACGATTTTTGACTTCGACT | | | | | | | RHTIATIFDFDSWG | | | | | | |
| | | CCTGGGGCCAGGGAACCCTGGTCACCGTCTCCTCA | | | | | | | QGTLVTVSS | | | | | | |
| ADI-81677 | 1287 | GAGGTGCAGCTGGTGGAGTCTGGGGGAGGCCTGGTCAAGCC | | | | | | 7145 | EVQLVESGGGLVKP | | 7496 | 6 | | 6 | |
| | | TGGGGGGTCCCTGAGACTCTCCTGTGCAGCCTCTGGATTCA | | | | | | | GGSLRLSCAASGFT | | | | | | |
| | | CCTTCAGTACCTATACCATGAACTGGGTCCGCCAGGCTCCA | | | | | | | FSTYTMNWVRQAPG | | | | | | |
| | | GGGAAGGGGCTGGAGTGGGTCTCATCCATTAGTAGTAGTAA | | | | | | | KGLEWVSSISSSNN | | | | | | |
| | | TAATTTCATATACTACGCAGACTCAGTCAAGGGCCGATTCA | | | | | | | FIYYADSVKGRFTI | | | | | | |
| | | CCATCTCCAGAGACAACGCCAAGAACTCAGTGTATCTGCAA | | | | | | | SRDNAKNSVYLQMN | | | | | | |
| | | ATGAACAGCCTGAGAGCCGAGGACACGGCTGTGTATTACTG | | | | | | | SLRAEDTAVYYCAK | | | | | | |
| | | TGCGAAAGATCTAACCTACTACTATGATAGTAGTGATTATT | | | | | | | DLTYYYDSSDYYGP | | | | | | |
| | | ACGGGCCTAATGCTTTTGATATCTGGGGCCAAGGGACCACG | | | | | | | NAFDIWGQGTTVTV | | | | | | |
| | | GTCACCGTCTCCTCA | | | | | | | SS | | | | | | |
| ADI-81790 | 1287 | GAGGTGCAGCTGTTGGAGTCTGGGGGAGGCGTGGTCCAGCC | | | | | | 7146 | EVQLLESGGGVVQP | | 7497 | 6 | | 6 | |
| | | TGGGACGTCCCTGAGACTCTCCTGTGCAGCCTCTGGATTCA | | | | | | | GTSLRLSCAASGFT | | | | | | |
| | | CCTTCAGGAGCTATGCTATGCACTGGGTCCGCCAGGCTCCA | | | | | | | FRSYAMHWVRQAPG | | | | | | |
| | | GGCAAGGGGCTAGAGTGGGTGGCAGCTATATCATATGATGG | | | | | | | KGLEWVAAISYDGS | | | | | | |
| | | AAGTAATAAATACTTCGCAGACTCCGTGAAGGGCCGATTCA | | | | | | | NKYFADSVKGRFTI | | | | | | |
| | | CCATCTCCAGAGACCATTCCAAGAACACGCTGTATCTGCAA | | | | | | | SRDHSKNTLYLQMN | | | | | | |
| | | ATGAACAGCCTGAGAGCTGCGGACACGGCTGTGTATTACTG | | | | | | | SLRAADTAVYYCAR | | | | | | |
| | | TGCGAGAGATCCTAGTGGTACTATGGTTCGGGGAGGTATTG | | | | | | | DPSGTMVRGGIVYY | | | | | | |
| | | TATACTATCACTACGGTATGGACGTCTGGGGCCAAGGGACC | | | | | | | HYGMDVWGQGTTVT | | | | | | |
| | | ACGGTCACCGTCTCCTCA | | | | | | | VSS | | | | | | |
| ADI-81755 | 1286 | GAGGTGCAGCTACAGCAGTGGGGCGCAGGACTGTTGAAGCC | | | | | | 7147 | EVQLQQWGAGLLKP | | 7498 | 5 | | 5 | |
| | | TTCGGAGACCCTGTCCCGCACCTGCGCTGTCTATGGTGGGT | | | | | | | SETLSRTCAVYGGS | | | | | | |
| | | CCTTCAGTGGTTACTACTGGAGCTGGATCCGCCAGTCCCCA | | | | | | | FSGYYWSWIRQSPG | | | | | | |
| | | GGGAAGGGGCTGGAGTGGATTGGGGAAATCAATCATAGTGG | | | | | | | KGLEWIGEINHSGS | | | | | | |
| | | AAGCACCAACTACAACCCGTCCCTCAAGAGTCGAGTCACCA | | | | | | | TNYNPSLKSRVTIS | | | | | | |
| | | TATCAGTAGACACGTCCAAGAGCCAGTTCTCCCTGAAGCTG | | | | | | | VDTSKSQFSLKLNS | | | | | | |
| | | AACTCTGTGACCGACGCGGACACGGCTGTATATTACTGTGC | | | | | | | VTDADTAVYYCARG | | | | | | |
| | | GAGAGGCAAAGCCATCTACGATTTTTGGCGTGGTTATAATG | | | | | | | KAIYDFWRGYNGRA | | | | | | |
| | | GCCGCGCCCTTAACTTTGACAACTGGGGCCAGGGAACCCTG | | | | | | | LNFDNWGQGTLVTV | | | | | | |
| | | GTCACCGTCTCCTCA | | | | | | | SS | | | | | | |
| ADI-81893 | 1287 | CAGGTGCAGCTGTTGGAGTCTGGGGCTGAGGTGAAGAAGCC | | | | | | 7148 | QVQLLESGAEVKKP | | 7499 | 5 | | 5 | |
| | | TGGGTCCTCGGTGAAGGTCTCCTGCAAGGCTTCTGGAGGCA | | | | | | | GSSVKVSCKASGGS | | | | | | |
| | | GCTTCAGCAGTGATGCTATCAACTGGGTGCGACAGGCCCCT | | | | | | | FSSDAINWVRQAPG | | | | | | |
| | | GGACAAGGGCTTGAGTGGATGGGAGGGATCAACCCTATTTT | | | | | | | QGLEWMGGINPIFG | | | | | | |
| | | TGGTACAGCAAACTACGCACAGAAGTTCCGGGGCAGAGTCA | | | | | | | TANYAQKFRGRVTI | | | | | | |
| | | CGATTACCGCGGACGAATCCACGAGCACAGCCTACATGGAG | | | | | | | TADESTSTAYMELS | | | | | | |
| | | CTGAGCAGCCTGAGATCTGAGGACACGGCCGTGTACTATTG | | | | | | | SLRSEDTAVYYCAK | | | | | | |
| | | TGCAAAAGATCAGGTTACTTCGCCCCTCTACGGGATGGACG | | | | | | | DQVTSPLYGMDVWG | | | | | | |
| | | TCTGGGGCCAAGGGACCACGGTCACCGTCTCCTCA | | | | | | | QGTTVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81649 | 22 | IGHV4- | EVQLL | 5335 | DSISSY | 320 | WIRQPP | 5862 | YIYYSG | 6145 | RVTISV | 6449 | ARGGGYG | 6769 | WGQGTL |
| | | 59 | ESGPG | | YWS | | GKGLEW | | STDYNP | | DTSKNQ | | DYFDY | | VTVSS |
| | | | LVKPS | | | | VG | | SLKS | | FYLKLS | | | | |
| | | | ETLSL | | | | | | | | SVTAAD | | | | |
| | | | TCTVS | | | | | | | | TAVYYC | | | | |
| | | | G | | | | | | | | | | | | |
| ADI-81716 | 86 | IGHV1- | QVQLV | 50 | GTFSSY | 5750 | WVRQAP | 5863 | RIIPLL | 6146 | RVTITA | 6450 | ARSAPLA | 6770 | WGQGTL |
| | | 69 | QSGAE | | AIN | | RQGLEW | | GIANYA | | DKSTST | | GTPPANW | | VTVSS |
| | | | VKKPG | | | | MG | | QKFQG | | AYMEVS | | FDP | | |
| | | | SSVKV | | | | | | | | SLRSED | | | | |
| | | | SCKAS | | | | | | | | TAVYYC | | | | |
| | | | G | | | | | | | | | | | | |
| ADI-81910 | 84 | IGHV3- | EVQLV | 81 | LTVSSN | 380 | WVRQTP | 448 | VIYSGG | 689 | RFTISR | 900 | ARMPYGM | 6771 | WGQGTT |
| | | 53 | ETGGG | | YMS | | GKGLEW | | STFYAD | | DKSKNT | | DV | | VTVSS |
| | | | LIQPG | | | | VS | | SVKG | | LYLQMN | | | | |
| | | | GSLRL | | | | | | | | SLRAED | | | | |
| | | | SCAAS | | | | | | | | TAVYYC | | | | |
| | | | G | | | | | | | | | | | | |
| ADI-81862 | 40 | IGHV3- | EVQLL | 5324 | FTFDDY | 5751 | WVRQAP | 437 | GISWNS | 6147 | RFTISR | 6422 | AKEGCSS | 6772 | WGQGTL |
| | | 9 | ESGGG | | ALH | | GKGLEW | | VSIDYA | | DNAKNS | | TSCYDY | | VTVSS |
| | | | LVQPG | | | | VS | | DSVKG | | LYLQMN | | | | |
| | | | RSLRL | | | | | | | | SLRAED | | | | |
| | | | SCAAS | | | | | | | | TALYYC | | | | |
| | | | G | | | | | | | | | | | | |
| ADI-81898 | 73 | IGHV5- | QVQLV | 174 | YDFSNL | 5752 | WVRLMP | 5864 | IISPPD | 6148 | QVTISV | 6451 | ARHLGVE | 6773 | WGQGTL |
| | | 51 | QSGAE | | WIG | | GKGPEW | | SDTRYN | | DNSINT | | LGTIRGY | | VTVSS |
| | | | VKKPG | | | | MG | | PSFEG | | AYLQWN | | FDS | | |
| | | | ESLKI | | | | | | | | SLKASD | | | | |
| | | | SCKGS | | | | | | | | TAIYYC | | | | |
| | | | G | | | | | | | | | | | | |
| ADI-81585 | 46 | IGHV4- | QVQLQ | 5503 | ESFRDY | 5753 | WIRQPP | 5865 | EVTHTG | 6149 | RVTISV | 6452 | ARGDFYS | 6774 | WGQGTT |
| | | 34 | QWGAG | | FWN | | GQGLEW | | NINYNP | | DTSKNQ | | TRLAFDI | | VTVSS |
| | | | LLKPS | | | | MG | | SLKS | | ISLNLR | | | | |
| | | | ETLSL | | | | | | | | SVSAAD | | | | |
| | | | TCAVY | | | | | | | | TAVYYC | | | | |
| | | | D | | | | | | | | | | | | |
| ADI-81613 | 70 | IGHV4- | QVQLQ | 5504 | DSISGY | 5707 | WIRQPP | 445 | YIYNSE | 6150 | RVTISV | 6453 | ARGAQYG | 6775 | WGQGTL |
| | | 4 | QSGPG | | YWS | | GKGLEW | | TTEYNP | | DTSKNQ | | DFFDY | | VTVSS |
| | | | LGLVK | | | | IG | | SLKS | | ISLKLS | | | | |
| | | | PSETL | | | | | | | | SVTAAD | | | | |
| | | | PLTCT | | | | | | | | TAVYYC | | | | |
| | | | VSG | | | | | | | | | | | | |
| ADI-81690 | 60 | IGHV3- | EVQLV | 5505 | FTFSSY | 5754 | WVRQAP | 472 | LISYDG | 6151 | RFTISR | 6454 | ARAKQWV | 6776 | WGQGTT |
| | | 30 | GVWGR | | EIH | | GKGLEW | | SNKYYA | | DNSKNT | | QRGYYYP | | VTVSS |
| | | | RGPAG | | | | VT | | DSVEG | | LYLQMN | | MDV | | |
| | | | RSLRL | | | | | | | | SLRPED | | | | |
| | | | SCAAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81649 | 1286 | GAGGTGCAGCTGTTGGAGTCGGGCCCAGGACTGGTGAAGCC | | | | | | 7149 | EVQLLESGPGLVKP | | 7500 | 4 | | 4 | |
| | | TTCGGAGACCCTGTCCCTCACCTGCACTGTCTCTGGTGACT | | | | | | | SETLSLTCTVSGDS | | | | | | |
| | | CCATCAGTAGTTACTACTGGAGCTGGATCCGGCAGCCCCCA | | | | | | | ISSYYWSWIRQPPG | | | | | | |
| | | GGGAAGGGACTGGAATGGGTTGGGTATATCTATTACAGTGG | | | | | | | KGLEWVGYIYYSGS | | | | | | |
| | | GAGCACCGACTACAACCCCTCCCTCAAGAGTCGAGTCACCA | | | | | | | TDYNPSLKSRVTIS | | | | | | |
| | | TATCAGTAGACACGTCCAAGAACCAATTCTACCTGAAGCTG | | | | | | | VDTSKNQFYLKLSS | | | | | | |
| | | AGCTCTGTGACCGCTGCGGACACGGCCGTGTATTATTGTGC | | | | | | | VTAADTAVYYCARG | | | | | | |
| | | GAGAGGGGGTGGCTACGGTGACTACTTTGACTACTGGGGCC | | | | | | | GGYGDYFDYWGQGT | | | | | | |
| | | AGGGAACCCTGGTCACCGTCTCTTCA | | | | | | | LVTVSS | | | | | | |
| ADI-81716 | 1286 | TGGGTCCTCGGTGAAGGTCTCCTGCAAGGCTTCTGGAGGCA | | | | | | 7150 | QVQLVQSGAEVKKP | | 7501 | 4 | | 4 | |
| | | CCTTCAGCAGTTATGCTATCAACTGGGTGCGACAGGCCCCT | | | | | | | GSSVKVSCKASGGT | | | | | | |
| | | AGACAAGGGCTTGAGTGGATGGGTAGGATCATCCCTCTCCT | | | | | | | FSSYAINWVRQAPR | | | | | | |
| | | TGGTATAGCAAACTACGCACAGAAGTTCCAGGGCAGAGTCA | | | | | | | QGLEWMGRIIPLLG | | | | | | |
| | | CGATTACCGCGGACAAATCCACGAGCACAGCCTACATGGAG | | | | | | | IANYAQKFQGRVTI | | | | | | |
| | | GTGAGCAGCCTAAGATCTGAGGACACGGCCGTGTATTACTG | | | | | | | TADKSTSTAYMEVS | | | | | | |
| | | TGCGCGTTCTGCCCCCCTTGCTGGAACTCCGCCGGCCAACT | | | | | | | SLRSEDTAVYYCAR | | | | | | |
| | | GGTTCGACCCCTGGGGCCAGGGAACCCTGGTCACCGTCTCC | | | | | | | SAPLAGTPPANWFD | | | | | | |
| | | TCA | | | | | | | PWGQGTLVTVSS | | | | | | |
| ADI-81910 | 1287 | GAGGTGCAGCTGGTGGAGACTGGAGGAGGCTTGATCCAGCC | | | | | | 7151 | EVQLVETGGGLIQP | | 7502 | 4 | | 4 | |
| | | TGGGGGGTCCCTGAGACTCTCCTGTGCAGCCTCTGGGCTCA | | | | | | | GGSLRLSCAASGLT | | | | | | |
| | | CCGTCAGTAGCAACTACATGAGCTGGGTCCGCCAGACTCCA | | | | | | | VSSNYMSWVRQTPG | | | | | | |
| | | GGGAAGGGGCTGGAGTGGGTCTCAGTTATTTATAGCGGTGG | | | | | | | KGLEWVSVIYSGGS | | | | | | |
| | | TAGCACATTCTACGCAGACTCCGTTAAGGGCCGATTCACCA | | | | | | | TFYADSVKGRFTIS | | | | | | |
| | | TCTCCAGAGACAAATCCAAGAACACGCTGTATCTTCAAATG | | | | | | | RDKSKNTLYLQMNS | | | | | | |
| | | AACAGCTTGAGAGCCGAGGACACGGCCGTGTATTATTGTGC | | | | | | | LRAEDTAVYYCARM | | | | | | |
| | | GAGAATGCCTTACGGTATGGACGTCTGGGGCCAAGGGACCA | | | | | | | PYGMDVWGQGTTVT | | | | | | |
| | | CGGTCACCGTCTCCTCA | | | | | | | VSS | | | | | | |
| ADI-81862 | 1286 | GAGGTGCAGCTGTTGGAGTCTGGGGGAGGCTTGGTACAGCC | | | | | | 7152 | EVQLLESGGGLVQP | | 7503 | 3 | | 3 | |
| | | TGGCAGGTCCCTGAGACTCTCCTGTGCAGCCTCTGGATTCA | | | | | | | GRSLRLSCAASGFT | | | | | | |
| | | CCTTTGATGATTATGCCTTGCACTGGGTCCGGCAAGCTCCA | | | | | | | FDDYALHWVRQAPG | | | | | | |
| | | GGGAAGGGCCTGGAGTGGGTCTCAGGTATTAGTTGGAATAG | | | | | | | KGLEWVSGISWNSV | | | | | | |
| | | TGTTAGCATAGACTATGCGGACTCTGTGAAGGGCCGATTCA | | | | | | | SIDYADSVKGRFTI | | | | | | |
| | | CCATCTCCAGAGACAACGCCAAGAACTCCCTGTATCTGCAA | | | | | | | SRDNAKNSLYLQMN | | | | | | |
| | | ATGAACAGTCTGAGAGCTGAGGACACGGCCTTATATTACTG | | | | | | | SLRAEDTALYYCAK | | | | | | |
| | | TGCAAAAGAGGGATGTAGTAGTACCAGCTGCTACGACTACT | | | | | | | EGCSSTSCYDYWGQ | | | | | | |
| | | GGGGCCAGGGAACCCTGGTCACCGTCTCCTCA | | | | | | | GTLVTVSS | | | | | | |
| ADI-81898 | 1286 | CGGGGAGTCTCTGAAGATCTCCTGTAAGGGTTCTGGATACG | | | | | | 7153 | QVQLVQSGAEVKKP | | 7504 | 20 | | 15 | |
| | | ACTTTAGCAACCTCTGGATCGGCTGGGTGCGCCTGATGCCC | | | | | | | GESLKISCKGSGYD | | | | | | |
| | | GGGAAAGGCCCGGAGTGGATGGGGATCATCTCTCCTCCCGA | | | | | | | FSNLWIGWVRLMPG | | | | | | |
| | | CTCTGATACCAGATACAACCCGTCCTTCGAAGGCCAGGTCA | | | | | | | KGPEWMGIISPPDS | | | | | | |
| | | CCATCTCAGTCGACAACTCCATCAATACCGCCTACCTGCAG | | | | | | | DTRYNPSFEGQVTI | | | | | | |
| | | TGGAACAGCCTGAAGGCCTCGGACACCGCCATCTATTACTG | | | | | | | SVDNSINTAYLQWN | | | | | | |
| | | TGCGAGACACTTAGGAGTAGAATTGGGTACAATACGCGGTT | | | | | | | SLKASDTAIYYCAR | | | | | | |
| | | ATTTTGACTCCTGGGGCCAGGGAACCCTGGTCACCGTCTCC | | | | | | | HLGVELGTIRGYFD | | | | | | |
| | | TCA | | | | | | | SWGQGTLVTVSS | | | | | | |
| ADI-81585 | 1287 | CAGGTGCAGCTACAGCAGTGGGGCGCAGGGCTGTTGAAGCC | | | | | | 7154 | QVQLQQWGAGLLKP | | 7505 | 18 | | 17 | |
| | | TTCGGAGACCCTGTCCCTCACTTGTGCTGTCTATGATGAGT | | | | | | | SETLSLTCAVYDES | | | | | | |
| | | CCTTCAGAGATTACTTCTGGAATTGGATCCGGCAACCCCCA | | | | | | | FRDYFWNWIRQPPG | | | | | | |
| | | GGTCAGGGGCTGGAGTGGATGGGGGAAGTCACTCATACTGG | | | | | | | QGLEWMGEVTHTGN | | | | | | |
| | | AAACATCAACTACAACCCGTCCCTTAAGAGTCGAGTCACCA | | | | | | | INYNPSLKSRVTIS | | | | | | |
| | | TATCCGTGGACACGTCCAAGAACCAAATCTCCCTGAACCTG | | | | | | | VDTSKNQISLNLRS | | | | | | |
| | | AGGTCTGTGAGCGCCGCGGACACGGCCGTTTATTACTGTGC | | | | | | | VSAADTAVYYCARG | | | | | | |
| | | GAGAGGCGATTTTTATAGCACCAGGCTCGCTTTTGATATCT | | | | | | | DFYSTRLAFDIWGQ | | | | | | |
| | | GGGGCCAAGGGACCACGGTCACCGTCTCCTCA | | | | | | | GTTVTVSS | | | | | | |
| ADI-81613 | 1286 | CAGGTGCAGCTGCAGCAGTCGGGCCCAGGACTAGGACTGGT | | | | | | 7155 | QVQLQQSGPGLGLV | | 7506 | 17 | | 10 | |
| | | GAAGCCCTCGGAGACCCTGCCCCTCACCTGCACTGTCTCTG | | | | | | | KPSETLPLTCTVSG | | | | | | |
| | | GTGACTCCATCAGTGGTTACTACTGGAGCTGGATCCGGCAG | | | | | | | DSISGYYWSWIRQP | | | | | | |
| | | CCCCCAGGGAAGGGACTGGAGTGGATTGGCTATATCTATAA | | | | | | | PGKGLEWIGYIYNS | | | | | | |
| | | TAGTGAGACTACTGAGTACAACCCCTCCCTCAAGAGTCGAG | | | | | | | ETTEYNPSLKSRVT | | | | | | |
| | | TCACCATCTCAGTAGACACGTCCAAGAACCAGATCTCCCTG | | | | | | | ISVDTSKNQISLKL | | | | | | |
| | | AAGCTGAGCTCTGTGACCGCCGCAGACACGGCCGTGTATTA | | | | | | | SSVTAADTAVYYCA | | | | | | |
| | | CTGTGCGAGAGGGGCTCAGTACGGCGACTTCTTTGACTACT | | | | | | | RGAQYGDFFDYWGQ | | | | | | |
| | | GGGGCCAGGGAACCCTGGTCACCGTCTCCTCA | | | | | | | GTLVTVSS | | | | | | |
| ADI-81690 | 1287 | TGGGAGGTCCCTGAGACTCTCCTGTGCAGCCTCTGGATTCA | | | | | | 7156 | EVQLVGVWGRRGPA | | 7507 | 17 | | 11 | |
| | | CCTTCAGTAGCTATGAAATCCACTGGGTCCGCCAGGCTCCA | | | | | | | GRSLRLSCAASGFT | | | | | | |
| | | GGCAAGGGGCTGGAGTGGGTGACACTTATATCATATGACGG | | | | | | | FSSYEIHWVRQAPG | | | | | | |
| | | AAGCAATAAATACTACGCAGACTCCGTGGAGGGCCGATTCA | | | | | | | KGLEWVTLISYDGS | | | | | | |
| | | CCATCTCCAGAGACAATTCCAAGAACACGCTGTATCTGCAA | | | | | | | NKYYADSVEGRFTI | | | | | | |
| | | ATGAACAGCCTGAGACCTGAGGACACGGCTGTGTATTACTG | | | | | | | SRDNSKNTLYLQMN | | | | | | |
| | | TGCGAGAGCAAAGCAGTGGGTGCAACGGGGGTACTACTACC | | | | | | | SLRPEDTAVYYCAR | | | | | | |
| | | CTATGGACGTCTGGGGCCAAGGAACCACGGTCACCGTCTCC | | | | | | | AKQWVQRGYYYPMD | | | | | | |
| | | TCA | | | | | | | VWGQGTTVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81784 | 60 | IGHV3- | EVQLL | 5373 | FTFSHY | 5755 | WVRQAP | 431 | LISYDG | 6152 | RFTISR | 6455 | AKRVDTY | 6777 | WGQGTL |
| | | 30 | ESGGG | | GMH | | GKGLEW | | TKTSYA | | DNSKNT | | CYADCLG | | VTVSS |
| | | | VVQPG | | | | VA | | DSVKG | | LLLEMN | | FPTTLDY | | |
| | | | RSLRL | | | | | | | | SLRGED | | | | |
| | | | SCAAS | | | | | | | | TAVYYC | | | | |
| | | | G | | | | | | | | | | | | |
| ADI-81863 | 41 | IGHV1- | EVQLV | 5385 | YTFTTY | 5756 | WVRQAP | 433 | IINPDA | 6153 | RVTMTS | 6456 | ARDIFLV | 6778 | WGQGTT |
| | | 46 | ESGAE | | YVH | | GQGLEW | | GSTTYG | | DTSTST | | PAISAME | | VTVSS |
| | | | VKKPG | | | | MG | | QQFRG | | VYMELS | | V | | |
| | | | ASVKV | | | | | | | | SLRSED | | | | |
| | | | SCKAS | | | | | | | | TAVYYC | | | | |
| | | | G | | | | | | | | | | | | |
| ADI-81645 | 18 | IGHV4- | EVQLL | 5506 | GSFSNY | 5757 | WIRQPP | 5866 | YIFYSG | 6154 | RLTISV | 6457 | ARDLGYS | 6779 | WGQGTT |
| | | 59 | ESGPG | | YWS | | GKRLEW | | STNYNP | | DTSKNQ | | SGWPDAF | | VTVSS |
| | | | LVKLS | | | | IG | | SLRS | | FSLNLR | | DI | | |
| | | | ETLSL | | | | | | | | SVTAAD | | | | |
| | | | TCKVS | | | | | | | | TAVYYC | | | | |
| | | | G | | | | | | | | | | | | |
| ADI-81806 | 77 | IGHV3- | EVQLV | 5338 | FTFRRS | 5758 | WVRQAP | 431 | DVSSDG | 6155 | RFTISR | 6458 | ARDVDTA | 6780 | WGQGTL |
| | | 30 | ESGGG | | VMH | | GKGLEW | | SIKTYA | | DNSKYT | | MVTILDY | | VTVSS |
| | | | VVQPG | | | | VA | | DSVKG | | LYLQMN | | | | |
| | | | RSLRL | | | | | | | | SLRAED | | | | |
| | | | SCAAS | | | | | | | | TAVYYC | | | | |
| | | | G | | | | | | | | | | | | |
| ADI-81801 | 77 | IGHV3- | EVQLL | 5373 | FTFGSY | 5759 | WVRQAP | 431 | DVSSDG | 6155 | RFTISR | 6458 | ARDVDTA | 6780 | WGQGTL |
| | | 30 | ESGGG | | VMH | | GKGLEW | | SIKTYA | | DNSKYT | | MVTILDY | | VTVSS |
| | | | VVQPG | | | | VA | | DSVKG | | LYLQMN | | | | |
| | | | RSLRL | | | | | | | | SLRAED | | | | |
| | | | SCAAS | | | | | | | | TAVYYC | | | | |
| | | | G | | | | | | | | | | | | |
| ADI-81769 | 45 | IGHV3- | QVQLL | 74 | VTVSAN | 5760 | WVRQAP | 437 | IIYSGG | 6156 | RFTISR | 777 | ARDLEQF | 6781 | WGRGTL |
| | | 66 | ESGGG | | YMR | | GKGLEW | | STFYAD | | DNSKNT | | GIDL | | VTVSS |
| | | | LVQPG | | | | VS | | SVKD | | LYLQMN | | | | |
| | | | GSLRL | | | | | | | | SLRAED | | | | |
| | | | SCAAS | | | | | | | | TAVYYC | | | | |
| | | | G | | | | | | | | | | | | |
| ADI-81625 | 81 | IGHV3- | QVQLV | 10 | FTFRSY | 198 | WVRQAP | 431 | VILYDG | 6157 | RFTISR | 6459 | AKQMGSH | 6782 | WGQGTL |
| | | 30 | QSGGG | | GMH | | GKGLEW | | SDKYYA | | DNSKNT | | CSAGNCY | | VTVSS |
| | | | VVQPG | | | | VA | | DSVKG | | LYLQMN | | SATFDY | | |
| | | | RSLRL | | | | | | | | SLKRED | | | | |
| | | | SCAAS | | | | | | | | TAVYYC | | | | |
| | | | G | | | | | | | | | | | | |
| ADI-81741 | 18 | IGHV3- | EVQLV | 5379 | ITVSSN | 300 | WVRQAP | 437 | VVYAGG | 6158 | RFTISR | 6460 | AREAYAF | 6783 | WGQGTT |
| | | 53 | ESGGG | | YMS | | GKGLEW | | STFYAD | | HNSKNT | | DI | | VTVSS |
| | | | LVQPG | | | | VS | | SVKG | | LYLQMN | | | | |
| | | | GSLRL | | | | | | | | SLRVED | | | | |
| | | | SCTAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81784 | 1286 | GAGGTGCAGCTGTTGGAGTCTGGGGGAGGCGTGGTCCAGCC | | | | | | 7157 | EVQLLESGGGVVQP | | 7508 | 14 | | 9 | |
| | | TGGGAGGTCCCTGAGACTCTCCTGCGCAGCCTCTGGATTCA | | | | | | | GRSLRLSCAASGFT | | | | | | |
| | | CCTTCAGTCACTATGGCATGCACTGGGTCCGCCAGGCTCCA | | | | | | | FSHYGMHWVRQAPG | | | | | | |
| | | GGCAAGGGGCTGGAGTGGGTGGCACTGATTTCATATGATGG | | | | | | | KGLEWVALISYDGT | | | | | | |
| | | AACCAAAACATCCTATGCAGACTCCGTGAAGGGCCGATTCA | | | | | | | KTSYADSVKGRFTI | | | | | | |
| | | CCATCTCCAGAGACAATTCGAAGAACACACTGCTCCTGGAA | | | | | | | SRDNSKNTLLLEMN | | | | | | |
| | | ATGAACAGCCTGAGAGGTGAAGACACGGCTGTGTATTACTG | | | | | | | SLRGEDTAVYYCAK | | | | | | |
| | | TGCGAAAAGGGTCGATACATATTGTTATGCTGATTGCTTGG | | | | | | | RVDTYCYADCLGFP | | | | | | |
| | | GATTTCCCACCACCCTTGACTATTGGGGCCAGGGAACCCTG | | | | | | | TTLDYWGQGTLVTV | | | | | | |
| | | GTCACCGTCTCCTCA | | | | | | | SS | | | | | | |
| ADI-81863 | 1287 | GAGGTGCAGCTGGTGGAGTCTGGGGCTGAGGTGAAGAAGCC | | | | | | 7158 | EVQLVESGAEVKKP | | 7509 | 13 | | 9 | |
| | | TGGGGCCTCAGTGAAAGTTTCCTGCAAGGCATCTGGATATA | | | | | | | GASVKVSCKASGYT | | | | | | |
| | | CGTTCACAACCTACTATGTTCACTGGGTGCGACAGGCCCCT | | | | | | | FTTYYVHWVRQAPG | | | | | | |
| | | GGACAAGGGCTTGAGTGGATGGGAATAATCAACCCTGATGC | | | | | | | QGLEWMGIINPDAG | | | | | | |
| | | TGGAAGCACCACGTACGGACAGCAATTCCGGGGCAGAGTCA | | | | | | | STTYGQQFRGRVTM | | | | | | |
| | | CCATGACCAGCGACACGTCCACGAGCACAGTCTACATGGAG | | | | | | | TSDTSTSTVYMELS | | | | | | |
| | | CTGAGTAGCCTGAGATCTGAGGACACGGCCGTATATTACTG | | | | | | | SLRSEDTAVYYCAR | | | | | | |
| | | TGCGAGAGATATTTTTTTAGTACCAGCCATAAGCGCTATGG | | | | | | | DIFLVPAISAMEVW | | | | | | |
| | | AAGTCTGGGGCCAGGGGACCACGGTCACCGTCTCCTCA | | | | | | | GQGTTVTVSS | | | | | | |
| ADI-81645 | 1287 | GAGGTGCAGCTGTTGGAGTCGGGCCCAGGACTGGTGAAGCT | | | | | | 7159 | EVQLLESGPGLVKL | | 7510 | 12 | | 10 | |
| | | TTCGGAGACCCTGTCCCTCACCTGCAAAGTCTCTGGTGGCT | | | | | | | SETLSLTCKVSGGS | | | | | | |
| | | CCTTCAGTAATTACTACTGGAGCTGGATTCGGCAGCCCCCC | | | | | | | FSNYYWSWIRQPPG | | | | | | |
| | | GGGAAGAGACTGGAATGGATTGGATATATCTTCTACAGTGG | | | | | | | KRLEWIGYIFYSGS | | | | | | |
| | | GAGCACCAACTACAACCCCTCCCTCAGGAGTCGACTCACCA | | | | | | | TNYNPSLRSRLTIS | | | | | | |
| | | TATCAGTAGACACGTCCAAGAACCAATTCTCCCTGAACCTG | | | | | | | VDTSKNQFSLNLRS | | | | | | |
| | | AGATCTGTGACCGCTGCGGACACGGCCGTGTATTACTGTGC | | | | | | | VTAADTAVYYCARD | | | | | | |
| | | GAGAGATCTCGGATATAGCAGTGGCTGGCCTGATGCTTTTG | | | | | | | LGYSSGWPDAFDIW | | | | | | |
| | | ATATCTGGGGCCAAGGGACCACGGTCACCGTCTCCTCA | | | | | | | GQGTTVTVSS | | | | | | |
| ADI-81806 | 1286 | GAGGTGCAGCTGGTGGAGTCCGGGGGAGGCGTGGTCCAGCC | | | | | | 7160 | EVQLVESGGGVVQP | | 7511 | 11 | | 10 | |
| | | TGGGAGGTCCCTGAGACTCTCCTGTGCAGCCTCTGGATTCA | | | | | | | GRSLRLSCAASGFT | | | | | | |
| | | CCTTCCGTAGGTCTGTTATGCACTGGGTCCGCCAGGCTCCA | | | | | | | FRRSVMHWVRQAPG | | | | | | |
| | | GGCAAGGGGCTGGAGTGGGTGGCAGATGTATCATCTGATGG | | | | | | | KGLEWVADVSSDGS | | | | | | |
| | | AAGCATTAAAACCTACGCAGACTCCGTGAAGGGCCGATTCA | | | | | | | IKTYADSVKGRFTI | | | | | | |
| | | CCATCTCCAGAGACAATTCCAAGTACACGCTGTATCTGCAA | | | | | | | SRDNSKYTLYLQMN | | | | | | |
| | | ATGAACAGCCTGAGAGCTGAGGACACGGCTGTGTATTACTG | | | | | | | SLRAEDTAVYYCAR | | | | | | |
| | | TGCGAGAGATGTGGATACAGCTATGGTTACTATTCTTGACT | | | | | | | DVDTAMVTILDYWG | | | | | | |
| | | ACTGGGGCCAGGGAACCCTGGTCACCGTCTCCTCA | | | | | | | QGTLVTVSS | | | | | | |
| ADI-81801 | 1286 | GAGGTGCAGCTGTTGGAGTCCGGGGGAGGCGTGGTCCAGCC | | | | | | 7161 | EVQLLESGGGVVQP | | 7512 | 9 | | 8 | |
| | | TGGGAGGTCCCTGAGACTCTCCTGTGCAGCCTCTGGATTTA | | | | | | | GRSLRLSCAASGFT | | | | | | |
| | | CCTTCGGTAGCTATGTTATGCACTGGGTCCGCCAGGCTCCA | | | | | | | FGSYVMHWVRQAPG | | | | | | |
| | | GGCAAGGGGCTGGAGTGGGTGGCAGATGTATCATCTGATGG | | | | | | | KGLEWVADVSSDGS | | | | | | |
| | | AAGCATTAAAACCTACGCAGACTCCGTGAAGGGCCGATTCA | | | | | | | IKTYADSVKGRFTI | | | | | | |
| | | CCATCTCCAGAGACAATTCCAAGTACACGCTGTATCTGCAA | | | | | | | SRDNSKYTLYLQMN | | | | | | |
| | | ATGAACAGCCTGAGAGCTGAGGACACGGCTGTGTATTACTG | | | | | | | SLRAEDTAVYYCAR | | | | | | |
| | | TGCGAGAGATGTGGATACAGCTATGGTTACTATTCTTGACT | | | | | | | DVDTAMVTILDYWG | | | | | | |
| | | ACTGGGGCCAGGGAACCCTGGTCACCGTCTCCTCA | | | | | | | QGTLVTVSS | | | | | | |
| ADI-81769 | 1288 | CAGGTGCAGCTGTTGGAGTCTGGGGGAGGGTTGGTCCAGCC | | | | | | 7162 | QVQLLESGGGLVQP | | 7513 | 8 | | 6 | |
| | | TGGGGGGTCCCTGAGACTCTCCTGTGCAGCCTCTGGAGTCA | | | | | | | GGSLRLSCAASGVT | | | | | | |
| | | CCGTCAGTGCCAACTACATGAGGTGGGTCCGCCAGGCTCCA | | | | | | | VSANYMRWVRQAPG | | | | | | |
| | | GGGAAGGGGCTGGAGTGGGTCTCAATTATTTATAGCGGTGG | | | | | | | KGLEWVSIIYSGGS | | | | | | |
| | | AAGCACATTTTACGCAGACTCCGTGAAGGACAGATTCACCA | | | | | | | TFYADSVKDRFTIS | | | | | | |
| | | TCTCCAGAGACAACTCCAAGAACACACTGTATCTTCAAATG | | | | | | | RDNSKNTLYLQMNS | | | | | | |
| | | AACAGCCTGCGAGCCGAGGACACGGCTGTCTATTACTGTGC | | | | | | | LRAEDTAVYYCARD | | | | | | |
| | | GAGAGACTTAGAACAGTTCGGGATCGATCTCTGGGGCCGTG | | | | | | | LEQFGIDLWGRGTL | | | | | | |
| | | GCACCCTGGTCACCGTCTCCTCA | | | | | | | VTVSS | | | | | | |
| ADI-81625 | 1286 | CAGGTGCAGCTGGTGCAGTCTGGGGGAGGCGTGGTCCAGCC | | | | | | 7163 | QVQLVQSGGGVVQP | | 7514 | 7 | | 5 | |
| | | TGGGAGGTCCCTGAGACTCTCCTGTGCAGCCTCTGGATTCA | | | | | | | GRSLRLSCAASGFT | | | | | | |
| | | CCTTCAGAAGTTATGGCATGCACTGGGTCCGCCAGGCTCCA | | | | | | | FRSYGMHWVRQAPG | | | | | | |
| | | GGCAAGGGGCTGGAGTGGGTGGCAGTTATATTATATGATGG | | | | | | | KGLEWVAVILYDGS | | | | | | |
| | | AAGTGACAAATACTATGCAGACTCCGTGAAGGGCCGATTCA | | | | | | | DKYYADSVKGRFTI | | | | | | |
| | | CCATCTCCAGAGACAATTCCAAGAACACGCTGTATCTGCAA | | | | | | | SRDNSKNTLYLQMN | | | | | | |
| | | ATGAACAGTCTGAAACGTGAGGACACGGCTGTGTACTACTG | | | | | | | SLKREDTAVYYCAK | | | | | | |
| | | TGCGAAACAGATGGGGTCACATTGTAGTGCTGGGAACTGCT | | | | | | | QMGSHCSAGNCYSA | | | | | | |
| | | ACTCAGCTACTTTTGACTACTGGGGCCAGGGAACCCTGGTC | | | | | | | TFDYWGQGTLVTVS | | | | | | |
| | | ACCGTCTCCTCA | | | | | | | S | | | | | | |
| ADI-81741 | 1287 | GAGGTGCAGCTGGTGGAGTCTGGAGGAGGCTTGGTCCAGCC | | | | | | 7164 | EVQLVESGGGLVQP | | 7515 | 7 | | 6 | |
| | | TGGGGGGTCCCTGAGACTCTCCTGTACAGCCTCTGGGATCA | | | | | | | GGSLRLSCTASGIT | | | | | | |
| | | CCGTCAGTAGCAACTACATGAGTTGGGTCCGCCAGGCTCCA | | | | | | | VSSNYMSWVRQAPG | | | | | | |
| | | GGGAAGGGGCTGGAGTGGGTCTCAGTTGTTTATGCCGGAGG | | | | | | | KGLEWVSVVYAGGS | | | | | | |
| | | TAGCACATTCTATGCAGACTCCGTGAAGGGCCGATTCACCA | | | | | | | TFYADSVKGRFTIS | | | | | | |
| | | TCTCCAGACACAATTCCAAGAACACACTGTATCTTCAAATG | | | | | | | RHNSKNTLYLQMNS | | | | | | |
| | | AACAGTCTCAGAGTTGAGGACACGGCCGTGTATTACTGTGC | | | | | | | LRVEDTAVYYCARE | | | | | | |
| | | GAGAGAGGCTTATGCCTTTGATATCTGGGGCCAAGGGACCA | | | | | | | AYAFDIWGQGTTVT | | | | | | |
| | | CGGTCACCGTCTCCTCA | | | | | | | VSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81761 | 37 | IGHV1- | EVQLV | 5507 | YTFTNY | 5761 | WVRQAP | 433 | IINPSA | 6159 | RVTMTT | 6461 | ARDIVQI | 6784 | WGQGTL |
| | | 46 | QSGAE | | YIH | | GQGLEW | | GSTSYA | | DTSTST | | TAAMGLE | | VTVSS |
| | | | VRKPG | | | | MG | | QKFQG | | VYMELS | | Y | | |
| | | | ASVKV | | | | | | | | SLRSED | | | | |
| | | | SCKAS | | | | | | | | TAIYYC | | | | |
| | | | G | | | | | | | | | | | | |
| ADI-81867 | 45 | IGHV3- | EVQLV | 5508 | FTFSSY | 199 | WVRQAP | 431 | VISYDG | 6160 | RFTISR | 6462 | ARGVRWN | 6785 | WGQGTL |
| | | 30 | ESGGG | | AMH | | GKGLEW | | HNKYYA | | DNSKNT | | GDYAVDY | | VTVSS |
| | | | VVQPG | | | | VA | | DSVKG | | LYLQMD | | | | |
| | | | RSLRV | | | | | | | | SLRADE | | | | |
| | | | SCAAS | | | | | | | | TAVYYC | | | | |
| | | | G | | | | | | | | | | | | |
| ADI-81655 | 28 | IGHV4- | EVQLL | 5509 | GSISSG | 5762 | WIRQHP | 450 | YIFYSG | 6161 | RVTISV | 6463 | ARGRQLI | 6786 | WGQGTL |
| | | 31 | ESGPG | | DYYWS | | GKGLEW | | STYYNP | | DTSKNQ | | NWFDP | | VTVSS |
| | | | LVRPS | | | | IG | | SLKS | | FSLKLN | | | | |
| | | | QTLSL | | | | | | | | SVTAAD | | | | |
| | | | TCTVS | | | | | | | | TAVYHC | | | | |
| | | | G | | | | | | | | | | | | |
| ADI-81791 | 67 | IGHV1- | QVQLV | 5510 | YTFTSY | 5763 | WVRQAP | 433 | IINPSG | 6162 | RVTMTR | 6464 | ARGGRGF | 6787 | WGQGTT |
| | | 46 | ESGAE | | YIH | | GQGLEW | | GSTTYA | | DTSTST | | LEGVLSD | | VTVSS |
| | | | VKKPG | | | | MG | | QKFQG | | VYMELS | | ENYYHYY | | |
| | | | ASVTV | | | | | | | | SLRSED | | GMDA | | |
| | | | SCKAP | | | | | | | | TAVYFC | | | | |
| | | | A | | | | | | | | | | | | |
| ADI-81842 | 20 | IGHV4- | EVQLL | 5511 | GSISSD | 5764 | WIRQPP | 445 | YIYHSE | 6163 | RVTISV | 6465 | AGEVVRG | 6788 | WGQGTP |
| | | 30-2 | ESGSG | | DYSWN | | GKGLEW | | STYYNP | | DRSKNQ | | NGRNAFH | | VTVSS |
| | | | LVKPS | | | | IG | | SLES | | FSLNLS | | I | | |
| | | | QTLSL | | | | | | | | SVTAAD | | | | |
| | | | TCAVS | | | | | | | | TAVYYC | | | | |
| | | | G | | | | | | | | | | | | |
| ADI-81846 | 24 | IGHV3- | EVQLL | 5373 | FTFSGY | 5765 | WVRQAP | 431 | VISYDG | 6164 | RFTISR | 6466 | ARDSHPP | 6789 | WGQGTL |
| | | 30 | ESGGG | | AMH | | GKGLEW | | INKYYA | | DNSMTT | | VHYFWSG | | VTVSS |
| | | | VVQPG | | | | VA | | DSVKG | | LFLQMN | | YYCDY | | |
| | | | RSLRL | | | | | | | | SLRAED | | | | |
| | | | SCAAS | | | | | | | | TAVYYC | | | | |
| | | | G | | | | | | | | | | | | |
| ADI-81894 | 16 | IGHV3- | QVQLV | 24 | FTFSSY | 5603 | WVRQAP | 437 | GVSGSG | 6165 | RFTISR | 777 | GKDSNLI | 6790 | WGQGTL |
| | | 23 | ESGGG | | AMS | | GKGLEW | | DGTFYA | | DNSKNT | | TMIVVDE | | VTVSS |
| | | | LVQPG | | | | VS | | DSVKG | | LYLQMN | | Y | | |
| | | | GSLRL | | | | | | | | SLRAED | | | | |
| | | | SCAAS | | | | | | | | TAVYYC | | | | |
| | | | G | | | | | | | | | | | | |
| ADI-81912 | 86 | IGHV3- | EVQLV | 5338 | FTFNNF | 5766 | WVRQAP | 431 | VISYDG | 6166 | RFTISR | 777 | ASAMVAH | 6791 | WGQGTT |
| | | 30 | ESGGG | | AFH | | GKGLEW | | SNKYYA | | DNSKNT | | YYYYYGM | | VTVSS |
| | | | VVQPG | | | | VA | | DSVKG | | LYLQMN | | DV | | |
| | | | RSLRL | | | | | | | | SLRAED | | | | |
| | | | SCAAS | | | | | | | | TAVYYC | | | | |
| | | | G | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81761 | 1286 | GAGGTGCAGCTGGTGCAGTCTGGGGCTGAGGTGAGGAAGCC | | | | | | 7165 | EVQLVQSGAEVRKP | | 7516 | 7 | | 6 | |
| | | TGGGGCCTCAGTGAAGGTTTCCTGCAAGGCATCTGGATACA | | | | | | | GASVKVSCKASGYT | | | | | | |
| | | CCTTCACCAACTACTATATACACTGGGTGCGACAGGCCCCT | | | | | | | FTNYYIHWVRQAPG | | | | | | |
| | | GGACAAGGGCTTGAGTGGATGGGAATAATCAACCCTAGTGC | | | | | | | QGLEWMGIINPSAG | | | | | | |
| | | TGGTAGCACAAGCTACGCACAGAAGTTCCAGGGCAGAGTCA | | | | | | | STSYAQKFQGRVTM | | | | | | |
| | | CCATGACCACGGACACGTCCACGAGCACAGTCTACATGGAG | | | | | | | TTDTSTSTVYMELS | | | | | | |
| | | CTGAGCAGCCTGAGATCTGAGGACACGGCCATATATTACTG | | | | | | | SLRSEDTAIYYCAR | | | | | | |
| | | TGCGAGAGATATTGTACAAATAACAGCTGCTATGGGGCTTG | | | | | | | DIVQITAAMGLEYW | | | | | | |
| | | AGTACTGGGGCCAGGGAACCCTGGTCACCGTCTCCTCA | | | | | | | GQGTLVTVSS | | | | | | |
| ADI-81867 | 1286 | GAGGTGCAGCTGGTGGAGTCTGGGGGAGGCGTGGTCCAGCC | | | | | | 7166 | EVQLVESGGGVVQP | | 7517 | 7 | | 5 | |
| | | TGGGAGGTCCCTGAGAGTCTCCTGTGCAGCCTCTGGATTCA | | | | | | | GRSLRVSCAASGFT | | | | | | |
| | | CCTTCAGTTCCTATGCTATGCACTGGGTCCGCCAGGCTCCA | | | | | | | FSSYAMHWVRQAPG | | | | | | |
| | | GGCAAGGGGCTGGAGTGGGTGGCAGTTATATCATATGATGG | | | | | | | KGLEWVAVISYDGH | | | | | | |
| | | ACACAATAAATACTACGCAGATTCCGTGAAGGGCCGATTCA | | | | | | | NKYYADSVKGRFTI | | | | | | |
| | | CCATCTCCAGAGACAATTCCAAGAACACGCTGTATCTGCAA | | | | | | | SRDNSKNTLYLQMD | | | | | | |
| | | ATGGACAGCCTGAGAGCTGACGAGACGGCTGTGTATTACTG | | | | | | | SLRADETAVYYCAR | | | | | | |
| | | TGCGAGAGGGGTAAGGTGGAACGGTGACTACGCCGTTGACT | | | | | | | GVRWNGDYAVDYWG | | | | | | |
| | | ACTGGGGCCAGGGAACCCTGGTCACCGTCTCCTCA | | | | | | | QGTLVTVSS | | | | | | |
| ADI-81655 | 1286 | GAGGTGCAGCTGTTGGAGTCGGGCCCAGGACTGGTGAGGCC | | | | | | 7167 | EVQLLESGPGLVRP | | 7518 | 6 | | 6 | |
| | | TTCACAGACCCTGTCCCTCACCTGCACTGTCTCTGGTGGCT | | | | | | | SQTLSLTCTVSGGS | | | | | | |
| | | CCATCAGCAGTGGCGATTACTACTGGAGCTGGATCCGCCAG | | | | | | | ISSGDYYWSWIRQH | | | | | | |
| | | CACCCAGGGAAGGGCCTGGAGTGGATTGGGTACATCTTTTA | | | | | | | PGKGLEWIGYIFYS | | | | | | |
| | | CAGTGGGAGCACCTATTACAACCCGTCCCTCAAGAGTCGAG | | | | | | | GSTYYNPSLKSRVT | | | | | | |
| | | TTACCATATCAGTAGACACGTCTAAGAACCAATTCTCCCTG | | | | | | | ISVDTSKNQFSLKL | | | | | | |
| | | AAGCTGAACTCTGTGACTGCCGCGGACACGGCCGTGTATCA | | | | | | | NSVTAADTAVYHCA | | | | | | |
| | | CTGTGCGAGAGGTCGACAGCTAATAAACTGGTTCGACCCCT | | | | | | | RGRQLINWFDPWGQ | | | | | | |
| | | GGGGCCAAGGAACCCTGGTCACCGTCTCCTCA | | | | | | | GTLVTVSS | | | | | | |
| ADI-81791 | 1287 | CAGGTGCAGCTGGTGGAGTCTGGGGCTGAGGTGAAGAAGCC | | | | | | 7168 | QVQLVESGAEVKKP | | 7519 | 6 | | 6 | |
| | | TGGGGCCTCAGTGACGGTTTCCTGCAAGGCACCTGCATACA | | | | | | | GASVTVSCKAPAYT | | | | | | |
| | | CCTTCACCAGCTACTATATACACTGGGTGCGACAGGCCCCT | | | | | | | FTSYYIHWVRQAPG | | | | | | |
| | | GGACAAGGGCTTGAGTGGATGGGAATAATCAACCCTAGTGG | | | | | | | QGLEWMGIINPSGG | | | | | | |
| | | TGGTAGCACAACCTACGCCCAGAAGTTCCAGGGCAGAGTCA | | | | | | | STTYAQKFQGRVTM | | | | | | |
| | | CCATGACCAGGGACACGTCCACGAGCACAGTCTACATGGAG | | | | | | | TRDTSTSTVYMELS | | | | | | |
| | | CTGAGCAGCCTGAGATCTGAGGACACGGCCGTGTATTTCTG | | | | | | | SLRSEDTAVYFCAR | | | | | | |
| | | TGCGAGAGGAGGGCGGGGATTTTTGGAGGGGGTATTATCGG | | | | | | | GGRGFLEGVLSDEN | | | | | | |
| | | ATGAGAATTACTACCACTACTACGGTATGGACGCCTGGGGC | | | | | | | YYHYYGMDAWGQGT | | | | | | |
| | | CAAGGGACCACGGTCACCGTCTCCTCA | | | | | | | TVTVSS | | | | | | |
| ADI-81842 | 6805 | GAGGTGCAGCTGTTGGAGTCCGGCTCAGGACTGGTGAAGCC | | | | | | 7169 | EVQLLESGSGLVKP | | 7520 | 6 | | 6 | |
| | | TTCACAGACCCTGTCCCTCACCTGCGCTGTCTCTGGTGGCT | | | | | | | SQTLSLTCAVSGGS | | | | | | |
| | | CCATCAGCAGTGATGATTATTCCTGGAACTGGATCCGGCAG | | | | | | | ISSDDYSWNWIRQP | | | | | | |
| | | CCACCAGGGAAGGGCCTGGAGTGGATTGGGTACATCTATCA | | | | | | | PGKGLEWIGYIYHS | | | | | | |
| | | TAGTGAGAGTACCTACTACAACCCGTCCCTCGAGAGTCGAG | | | | | | | ESTYYNPSLESRVT | | | | | | |
| | | TCACCATATCAGTCGACAGGTCCAAGAACCAGTTCTCCCTG | | | | | | | ISVDRSKNQFSLNL | | | | | | |
| | | AACCTCAGCTCTGTGACCGCCGCGGACACGGCCGTGTATTA | | | | | | | SSVTAADTAVYYCA | | | | | | |
| | | CTGTGCCGGAGAAGTCGTTAGGGGTAACGGACGGAATGCTT | | | | | | | GEVVRGNGRNAFHI | | | | | | |
| | | TTCATATCTGGGGCCAAGGGACACCGGTCACCGTCTCCTCA | | | | | | | WGQGTPVTVSS | | | | | | |
| ADI-81846 | 1286 | TGGGAGGTCCCTGAGACTCTCCTGTGCAGCCTCCGGATTCA | | | | | | 7170 | EVQLLESGGGVVQP | | 7521 | 5 | | 5 | |
| | | CCTTCAGTGGCTATGCTATGCACTGGGTCCGCCAGGCTCCA | | | | | | | GRSLRLSCAASGFT | | | | | | |
| | | GGCAAGGGCCTAGAGTGGGTGGCAGTTATATCATATGATGG | | | | | | | FSGYAMHWVRQAPG | | | | | | |
| | | AATTAATAAATACTACGCAGACTCCGTGAAGGGCCGATTCA | | | | | | | KGLEWVAVISYDGI | | | | | | |
| | | CCATCTCCAGAGACAATTCCATGACCACGCTGTTTCTGCAA | | | | | | | NKYYADSVKGRFTI | | | | | | |
| | | ATGAACAGCCTCAGAGCTGAGGACACGGCTGTGTATTACTG | | | | | | | SRDNSMTTLFLQMN | | | | | | |
| | | TGCGAGAGATTCCCACCCTCCTGTTCATTACTTTTGGAGTG | | | | | | | SLRAEDTAVYYCAR | | | | | | |
| | | GTTATTATTGCGACTACTGGGGCCAGGGAACCCTGGTCACC | | | | | | | DSHPPVHYFWSGYY | | | | | | |
| | | GTCTCCTCA | | | | | | | CDYWGQGTLVTVSS | | | | | | |
| ADI-81894 | 1286 | CAGGTGCAGCTGGTGGAGTCTGGGGGAGGCTTGGTACAGCC | | | | | | 7171 | QVQLVESGGGLVQP | | 7522 | 5 | | 5 | |
| | | TGGGGGGTCCCTAAGACTCTCCTGTGCAGCCTCTGGATTCA | | | | | | | GGSLRLSCAASGFT | | | | | | |
| | | CCTTTAGCAGCTATGCCATGAGCTGGGTCCGCCAGGCTCCA | | | | | | | FSSYAMSWVRQAPG | | | | | | |
| | | GGGAAGGGGCTGGAGTGGGTCTCAGGTGTTAGTGGTAGTGG | | | | | | | KGLEWVSGVSGSGD | | | | | | |
| | | TGATGGCACATTCTACGCAGACTCCGTGAAGGGCCGGTTCA | | | | | | | GTFYADSVKGRFTI | | | | | | |
| | | CCATCTCCAGAGACAATTCCAAGAACACGCTGTATCTGCAA | | | | | | | SRDNSKNTLYLQMN | | | | | | |
| | | ATGAACAGCCTGAGAGCCGAGGACACGGCCGTATATTACTG | | | | | | | SLRAEDTAVYYCGK | | | | | | |
| | | TGGGAAAGATAGTAACCTTATTACTATGATAGTAGTTGATG | | | | | | | DSNLITMIVVDEYW | | | | | | |
| | | AGTACTGGGGCCAGGGAACCCTGGTCACCGTCTCCTCA | | | | | | | GQGTLVTVSS | | | | | | |
| ADI-81912 | 1287 | GAGGTGCAGCTGGTGGAGTCTGGGGGAGGCGTGGTCCAGCC | | | | | | 7172 | EVQLVESGGGVVQP | | 7523 | 5 | | 4 | |
| | | TGGGAGGTCCCTGAGACTCTCCTGTGCAGCCTCTGGATTCA | | | | | | | GRSLRLSCAASGFT | | | | | | |
| | | CCTTCAATAACTTTGCTTTTCACTGGGTCCGCCAGGCTCCA | | | | | | | FNNFAFHWVRQAPG | | | | | | |
| | | GGCAAGGGGCTAGAGTGGGTGGCAGTTATATCATATGATGG | | | | | | | KGLEWVAVISYDGS | | | | | | |
| | | AAGTAATAAATACTACGCAGACTCCGTGAAGGGCCGATTCA | | | | | | | NKYYADSVKGRFTI | | | | | | |
| | | CCATCTCCAGAGACAATTCCAAGAACACGCTGTATCTGCAA | | | | | | | SRDNSKNTLYLQMN | | | | | | |
| | | ATGAACAGCCTGAGAGCTGAGGACACGGCTGTGTATTACTG | | | | | | | SLRAEDTAVYYCAS | | | | | | |
| | | TGCGAGCGCTATGGTGGCTCATTACTACTACTACTACGGTA | | | | | | | AMVAHYYYYYGMDV | | | | | | |
| | | TGGACGTCTGGGGCCAAGGGACCACGGTCACCGTCTCCTCA | | | | | | | WGQGTTVTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81848 | 26 | IGHV4- | EVQLL | 5346 | GSISSD | 5767 | WIRQHP | 450 | YIYYSG | 611 | RVTISV | 1009 | ARNSRFL | 6792 | WGQGTL |
| | | 31 | ESGPG | | IYYWS | | GKGLEW | | STYYNP | | DTSKNQ | | GTNYFDY | | VTVSS |
| | | | LVKPS | | | | IG | | SLKS | | FSLKLS | | | | |
| | | | QTLSL | | | | | | | | SVTAAD | | | | |
| | | | TCTVS | | | | | | | | TAVYYC | | | | |
| | | | G | | | | | | | | | | | | |
| ADI-81915 | 89 | IGHV4- | EVQLL | 5346 | DSISSG | 418 | WIRQHP | 450 | YIYYSG | 611 | RVTMSL | 6467 | ARDWGRG | 6793 | WGQGTT |
| | | 31 | ESGPG | | GYYWS | | GKGLEW | | STYYNP | | DTSKNQ | | SSSSPEH | | VTVSS |
| | | | LVKPS | | | | IG | | SLKS | | FSLKLS | | DAFDI | | |
| | | | QTLSL | | | | | | | | SVTAAD | | | | |
| | | | TCTVS | | | | | | | | TAVYYC | | | | |
| | | | G | | | | | | | | | | | | |
| ADI-81605 | 63 | IGHV3- | QVQLV | 45 | FTFSSY | 262 | WVRQAP | 431 | LIWYDG | 6167 | RFTISR | 777 | ARAVPGT | 6794 | WGQGTL |
| | | 33 | QSGGG | | GMH | | GKGLEW | | SNKYYA | | DNSKNT | | NYFDY | | VTVSS |
| | | | VVQPG | | | | VA | | DSVKG | | LYLQMN | | | | |
| | | | RSLRL | | | | | | | | SLRAED | | | | |
| | | | SCVAS | | | | | | | | TAVYYC | | | | |
| | | | G | | | | | | | | | | | | |
| ADI-81870 | 29 | IGHV4- | EVQLL | 5346 | GSISSG | 5768 | WIRQHP | 450 | YIYYSG | 611 | RVTISV | 1009 | ARTYYYD | 6795 | WGQGTL |
| | | 31 | ESGPG | | GYYWS | | GKGLEW | | STYYNP | | DTSKNQ | | SSGNYYQ | | VTVSS |
| | | | LVKPS | | | | IG | | SLKS | | FSLKLS | | YYFDY | | |
| | | | QTLSL | | | | | | | | SVTAAD | | | | |
| | | | TCTVS | | | | | | | | TAVYYC | | | | |
| | | | G | | | | | | | | | | | | |
| ADI-81902 | 76 | IGHV1- | QVQLV | 5512 | YTFTNY | 5761 | WVRQAP | 433 | LINPSG | 6168 | RVTLTR | 6468 | ARGGRGF | 6796 | WGQGTT |
| | | 46 | QSGAA | | YIH | | GQGLEW | | GSSNYA | | GTATNT | | LEKILSD | | VTVSS |
| | | | VKKPG | | | | MG | | QKFQG | | VYMELS | | EGYYDYF | | |
| | | | ASVKV | | | | | | | | SLRSED | | GMDV | | |
| | | | SCQAS | | | | | | | | MAVYYC | | | | |
| | | | G | | | | | | | | | | | | |
| ADI-81617 | 74 | IGHV3- | RSSWW | 5513 | FTFSSY | 5769 | WVRQAP | 5867 | ALSASG | 6169 | RFTISR | 6469 | AKQAGSP | 6797 | WGQGTL |
| | | 23 | QSGGG | | ALS | | GKGLEC | | DAPYYA | | DNSRNT | | PDYFDY | | VTVSS |
| | | | LVQPG | | | | VS | | DSVKG | | LYLQMN | | | | |
| | | | GPLRL | | | | | | | | SLRAED | | | | |
| | | | SCAAS | | | | | | | | TAVYYC | | | | |
| | | | G | | | | | | | | | | | | |
| ADI-81570 | 33 | IGHV3- | QVQLV | 5514 | FIVSSN | 260 | WVRQAP | 437 | VIYPGG | 649 | RFTISR | 6470 | ARDYGDF | 6798 | WGQGTL |
| | | 53 | ESGGG | | YMS | | GKGLEW | | STFYAD | | DSSKNT | | YFDF | | VTVSS |
| | | | LIQPG | | | | VS | | SVKG | | LFLQMN | | | | |
| | | | GSLRL | | | | | | | | SLRAED | | | | |
| | | | SCAAS | | | | | | | | TAVYYC | | | | |
| | | | E | | | | | | | | | | | | |
| | SEQ | | | | | | | SEQ | | | SEQ | VH | | VH Amino | |
| | ID | | | | | | | ID | | | ID | Nucleotide | | Acid | |
| Clone ID | NO: | VH DNA | | | | | | NO: | VH Protein | | NO: | Substitution | | Substitution | |
| ADI-81848 | 1286 | GAGGTGCAGCTGTTGGAGTCGGGCCCAGGACTGGTGAAGCC | | | | | | 7173 | EVQLLESGPGLVKP | | 7524 | 4 | | 3 | |
| | | TTCACAGACCCTGTCCCTCACCTGCACTGTCTCTGGTGGCT | | | | | | | SQTLSLTCTVSGGS | | | | | | |
| | | CCATCAGCAGTGATATTTACTACTGGAGCTGGATCCGCCAG | | | | | | | ISSDIYYWSWIRQH | | | | | | |
| | | CACCCAGGGAAGGGCCTGGAGTGGATTGGGTACATCTATTA | | | | | | | PGKGLEWIGYIYYS | | | | | | |
| | | CAGTGGGAGCACCTACTACAACCCGTCCCTCAAGAGTCGAG | | | | | | | GSTYYNPSLKSRVT | | | | | | |
| | | TTACCATATCAGTAGACACGTCTAAGAACCAGTTCTCCCTG | | | | | | | ISVDTSKNQFSLKL | | | | | | |
| | | AAGCTGAGCTCTGTGACTGCCGCGGACACGGCCGTGTATTA | | | | | | | SSVTAADTAVYYCA | | | | | | |
| | | CTGTGCGAGAAATTCGAGATTCTTAGGGACAAACTACTTTG | | | | | | | RNSRFLGTNYFDYW | | | | | | |
| | | ACTACTGGGGCCAGGGAACCCTGGTCACCGTCTCCTCA | | | | | | | GQGTLVTVSS | | | | | | |
| ADI-81915 | 1287 | GAGGTGCAGCTGTTGGAGTCGGGCCCAGGACTGGTGAAGCC | | | | | | 7174 | EVQLLESGPGLVKP | | 7525 | 4 | | 4 | |
| | | TTCACAGACCCTGTCCCTCACCTGCACTGTCTCTGGTGACT | | | | | | | SQTLSLTCTVSGDS | | | | | | |
| | | CCATCAGTAGTGGTGGTTACTACTGGAGCTGGATCCGCCAG | | | | | | | ISSGGYYWSWIRQH | | | | | | |
| | | CACCCAGGGAAGGGCCTGGAGTGGATTGGGTACATCTATTA | | | | | | | PGKGLEWIGYIYYS | | | | | | |
| | | CAGTGGGAGCACCTACTACAACCCGTCCCTCAAGAGTCGAG | | | | | | | GSTYYNPSLKSRVT | | | | | | |
| | | TTACCATGTCATTAGACACGTCTAAGAACCAGTTCTCCCTG | | | | | | | MSLDTSKNQFSLKL | | | | | | |
| | | AAGCTGAGCTCTGTGACTGCCGCGGACACGGCCGTGTATTA | | | | | | | SSVTAADTAVYYCA | | | | | | |
| | | CTGTGCGAGAGATTGGGGTCGCGGGTCTAGCAGCTCGCCGG | | | | | | | RDWGRGSSSSPEHD | | | | | | |
| | | AACATGATGCTTTTGATATCTGGGGCCAAGGGACCACGGTC | | | | | | | AFDIWGQGTTVTVS | | | | | | |
| | | ACCGTCTCCTCA | | | | | | | S | | | | | | |
| ADI-81605 | 1286 | CAGGTGCAGCTGGTGCAGTCTGGGGGAGGCGTGGTCCAGCC | | | | | | 7175 | QVQLVQSGGGVVQP | | 7526 | 2 | | 2 | |
| | | TGGGAGGTCCCTGAGACTCTCCTGTGTAGCGTCTGGATTCA | | | | | | | GRSLRLSCVASGFT | | | | | | |
| | | CCTTCAGTAGCTATGGCATGCACTGGGTCCGCCAGGCTCCA | | | | | | | FSSYGMHWVRQAPG | | | | | | |
| | | GGCAAGGGGCTGGAGTGGGTGGCACTTATATGGTATGATGG | | | | | | | KGLEWVALIWYDGS | | | | | | |
| | | AAGTAATAAATATTATGCAGACTCCGTGAAGGGCCGATTCA | | | | | | | NKYYADSVKGRFTI | | | | | | |
| | | CCATCTCCAGAGACAATTCCAAGAACACGCTGTATCTGCAA | | | | | | | SRDNSKNTLYLQMN | | | | | | |
| | | ATGAACAGCCTGAGAGCCGAGGACACGGCTGTGTATTACTG | | | | | | | SLRAEDTAVYYCAR | | | | | | |
| | | TGCGAGAGCGGTGCCTGGTACTAACTACTTTGACTACTGGG | | | | | | | AVPGTNYFDYWGQG | | | | | | |
| | | GCCAGGGAACCCTGGTCACCGTCTCCTCA | | | | | | | TLVTVSS | | | | | | |
| ADI-81870 | 1286 | GAGGTGCAGCTGTTGGAGTCGGGCCCAGGACTGGTGAAGCC | | | | | | 7176 | EVQLLESGPGLVKP | | 7527 | 1 | | 1 | |
| | | TTCACAGACCCTGTCCCTCACCTGCACTGTCTCTGGTGGCT | | | | | | | SQTLSLTCTVSGGS | | | | | | |
| | | CCATCAGCAGTGGTGGTTACTACTGGAGCTGGATCCGCCAG | | | | | | | ISSGGYYWSWIRQH | | | | | | |
| | | CACCCAGGGAAGGGCCTGGAGTGGATTGGGTACATCTATTA | | | | | | | PGKGLEWIGYIYYS | | | | | | |
| | | CAGTGGGAGCACCTACTACAACCCGTCCCTCAAGAGTCGAG | | | | | | | GSTYYNPSLKSRVT | | | | | | |
| | | TTACCATATCAGTAGACACGTCTAAGAACCAGTTCTCCCTG | | | | | | | ISVDTSKNQFSLKL | | | | | | |
| | | AAGCTGAGCTCTGTGACTGCCGCGGACACGGCCGTTTATTA | | | | | | | SSVTAADTAVYYCA | | | | | | |
| | | TTGTGCGAGGACGTATTACTATGATAGCAGTGGTAATTACT | | | | | | | RTYYYDSSGNYYQY | | | | | | |
| | | ACCAGTACTACTTTGACTACTGGGGCCAGGGAACCCTGGTC | | | | | | | YFDYWGQGTLVTVS | | | | | | |
| | | ACCGTCTCCTCA | | | | | | | S | | | | | | |
| ADI-81902 | 1287 | CAGGTCCAGCTGGTGCAGTCTGGGGCTGCGGTGAAGAAGCC | | | | | | 7177 | QVQLVQSGAAVKKP | | 7528 | 13 | | 12 | |
| | | TGGGGCCTCAGTGAAAGTTTCCTGTCAGGCATCTGGATACA | | | | | | | GASVKVSCQASGYT | | | | | | |
| | | CCTTCACTAATTACTATATTCACTGGGTGCGACAGGCCCCT | | | | | | | FTNYYIHWVRQAPG | | | | | | |
| | | GGACAAGGGCTTGAGTGGATGGGATTAATCAATCCTAGTGG | | | | | | | QGLEWMGLINPSGG | | | | | | |
| | | TGGGAGCTCAAACTACGCACAGAAGTTCCAGGGCAGAGTCA | | | | | | | SSNYAQKFQGRVTL | | | | | | |
| | | CCTTGACCAGGGGCACGGCCACGAACACAGTTTACATGGAG | | | | | | | TRGTATNTVYMELS | | | | | | |
| | | CTGAGCAGCCTGAGATCCGAGGACATGGCCGTGTATTATTG | | | | | | | SLRSEDMAVYYCAR | | | | | | |
| | | TGCGAGAGGAGGGCGGGGATTTTTGGAGAAAATTTTATCGG | | | | | | | GGRGFLEKILSDEG | | | | | | |
| | | ATGAGGGTTACTACGACTATTTCGGTATGGACGTCTGGGGC | | | | | | | YYDYFGMDVWGQGT | | | | | | |
| | | CAAGGGACCACGGTCACCGTCTCCTCA | | | | | | | TVTVSS | | | | | | |
| ADI-81617 | 1286 | AGGTCCAGCTGGTGGCAGTCTGGGGGAGGCTTGGTACAGCC | | | | | | 7178 | RSSWWQSGGGLVQP | | 7529 | 12 | | 9 | |
| | | TGGGGGGCCCCTGCGACTCTCCTGTGCAGCCTCTGGATTCA | | | | | | | GGPLRLSCAASGFT | | | | | | |
| | | CCTTTAGCAGCTATGCCTTGAGCTGGGTCCGCCAGGCTCCA | | | | | | | FSSYALSWVRQAPG | | | | | | |
| | | GGGAAGGGGCTGGAGTGCGTCTCAGCTCTTAGTGCTAGTGG | | | | | | | KGLECVSALSASGD | | | | | | |
| | | TGATGCCCCTTACTACGCAGACTCCGTGAAGGGCCGGTTCA | | | | | | | APYYADSVKGRFTI | | | | | | |
| | | CCATCTCCAGAGACAATTCCCGGAACACGCTGTATCTGCAA | | | | | | | SRDNSRNTLYLQMN | | | | | | |
| | | ATGAACAGCCTGAGAGCCGAGGACACGGCCGTATATTACTG | | | | | | | SLRAEDTAVYYCAK | | | | | | |
| | | TGCGAAACAAGCTGGTTCACCCCCCGACTACTTTGACTACT | | | | | | | QAGSPPDYFDYWGQ | | | | | | |
| | | GGGGCCAGGGAACCCTGGTCACCGTCTCCTCA | | | | | | | GTLVTVSS | | | | | | |
| ADI-81570 | 1286 | CAGGTGCAGCTGGTGGAGTCTGGAGGAGGCTTGATCCAGCC | | | | | | 7179 | QVQLVESGGGLIQP | | 7530 | 8 | | 6 | |
| | | TGGGGGGTCCCTGAGACTCTCCTGTGCAGCCTCTGAATTCA | | | | | | | GGSLRLSCAASEFI | | | | | | |
| | | TCGTCAGTAGCAACTACATGAGTTGGGTCCGCCAGGCTCCA | | | | | | | VSSNYMSWVRQAPG | | | | | | |
| | | GGGAAGGGGCTGGAGTGGGTCTCAGTTATTTATCCCGGTGG | | | | | | | KGLEWVSVIYPGGS | | | | | | |
| | | TAGCACATTCTACGCAGACTCCGTGAAGGGCCGATTCACCA | | | | | | | TFYADSVKGRFTIS | | | | | | |
| | | TCTCCAGAGACAGTTCCAAGAACACACTGTTTCTTCAAATG | | | | | | | RDSSKNTLFLQMNS | | | | | | |
| | | AACAGCCTGAGAGCCGAGGACACGGCCGTGTACTACTGTGC | | | | | | | LRAEDTAVYYCARD | | | | | | |
| | | GAGGGATTACGGTGACTTCTACTTTGACTTCTGGGGCCAGG | | | | | | | YGDFYFDFWGQGTL | | | | | | |
| | | GAACCCTGGTCACCGTCTCCTCA | | | | | | | VTVSS | | | | | | |
| | VH | VH | | SEQ | | SEQ | | SEQ | | SEQ | | SEQ | | SEQ | |
| | CDR3 | Germ- | VH | ID | VH | ID | VH | ID | VH | ID | VH | ID | VH | ID | VH |
| Clone ID | Lineage | line | FR1 | NO: | CDR1 | NO: | FR2 | NO: | CDR2 | NO: | FR3 | NO: | CDR3 | NO: | FR4 |
| ADI-81664 | 36 | IGHV3- | EVQLV | 5362 | FTFSSF | 5770 | WVRQAP | 437 | AISNSG | 6170 | RFTISR | 6471 | AKQAYSS | 6799 | WGQGTL |
| | | 23 | ESGGG | | AMS | | GKGLEW | | GSTYYA | | DNSKNT | | GWYAAGL | | VTVSS |
| | | | LIQPG | | | | VS | | DSVKG | | LYLQMN | | VFDY | | |
| | | | GSLRL | | | | | | | | SLRAED | | | | |
| | | | SCAAS | | | | | | | | TALYYC | | | | |
| | | | G | | | | | | | | | | | | |
| ADI-81666 | 18 | IGHV4- | EVQLL | 5335 | GSISNY | 5771 | WIRQPP | 445 | YIFYGG | 6171 | RLTISV | 6472 | ARDLGYS | 6779 | WGQGTT |
| | | 59 | ESGPG | | YWS | | GKGLEW | | STNYNP | | DTSKNQ | | SGWPDAF | | VTVSS |
| | | | LVKPS | | | | IG | | SLRS | | FSLRLS | | DI | | |
| | | | ETLSL | | | | | | | | SVTAAD | | | | |
| | | | TCTVS | | | | | | | | TAVYYC | | | | |
| | | | G | | | | | | | | | | | | |
| ADI-81809 | 83 | IGHV3- | EVQLL | 5373 | FTFSSY | 199 | WVRQAP | 431 | VISSDG | 6172 | RFTISR | 6473 | ARDAENL | 6800 | WGQGTL |
| | | 30 | ESGGG | | AMH | | GKGLEW | | SMKFYA | | DNSKNT | | VTEFDY | | VTVSS |
| | | | VVQPG | | | | VA | | DSVKG | | LFLQMN | | | | |
| | | | RSLRL | | | | | | | | SLRPED | | | | |
| | | | SCAAS | | | | | | | | TAVYYC | | | | |
| | | | G | | | | | | | | | | | | |
| ADI-81539 | 7 | IGHV1- | QVQLV | 35 | YTFTTY | 5772 | WVRLAP | 5868 | IINPSG | 6173 | RVTMTR | 881 | VREGCSS | 6801 | WGQGTL |
| | | 46 | QSGAE | | YMH | | GQGLEW | | GGTSYA | | DTSTST | | TSCFDY | | VTVSS |
| | | | VKKPG | | | | MG | | QKFQG | | VYMELS | | | | |
| | | | ASVKV | | | | | | | | SLRSED | | | | |
| | | | SCKAS | | | | | | | | TAVYYC | | | | |
| | | | G | | | | | | | | | | | | |
| ADI-81648 | 21 | IGHV3- | EVQLV | 5338 | FTFSSY | 262 | WVRQAP | 431 | VIWYDG | 679 | RFTISR | 777 | ARDKIIW | 6802 | WGKGTT |
| | | 33 | ESGGG | | GMH | | GKGLEW | | SNKYYA | | DNSKNT | | ESGYHKN | | VTVSS |
| | | | VVQPG | | | | VA | | DSVKG | | LYLQMN | | DV | | |
| | | | RSLRL | | | | | | | | SLRAED | | | | |
| | | | SCAAS | | | | | | | | TAVYYC | | | | |
| | | | G | | | | | | | | | | | | |

| | SEQ | | SEQ | | SEQ | VH | VH Amino |
|---|---|---|---|---|---|---|---|
| | ID | | ID | | ID | Nucleotide | Acid |
| Clone ID | NO: | VH DNA | NO: | VH Protein | NO: | Substitution | Substitution |
| ADI-81664 | 1286 | TGGGGGGTCCCTGAGACTCTCCTGTGCAGCCTCTGGATTCA | 7180 | EVQLVESGGGLIQP | 7531 | 6 | 4 |
| | | CCTTTAGCAGCTTTGCCATGAGCTGGGTCCGCCAGGCTCCA | | GGSLRLSCAASGFT | | | |
| | | GGGAAGGGGCTGGAGTGGGTCTCAGCTATTAGTAATAGTGG | | FSSFAMSWVRQAPG | | | |
| | | TGGTAGCACATACTACGCAGACTCCGTGAAGGGCCGCTTCA | | KGLEWVSAISNSGG | | | |
| | | CCATCTCCAGAGACAATTCCAAGAACACGCTGTATCTGCAA | | STYYADSVKGRFTI | | | |
| | | ATGAACAGCCTGAGAGCCGAGGACACGGCCCTTTATTACTG | | SRDNSKNTLYLQMN | | | |
| | | TGCGAAACAAGCGTATAGCAGTGGCTGGTACGCCGCCGGGT | | SLRAEDTALYYCAK | | | |
| | | TAGTATTTGACTACTGGGGCCAGGGAACCCTGGTCACCGTC | | QAYSSGWYAAGLVF | | | |
| | | TCCTCA | | DYWGQGTLVTVSS | | | |
| ADI-81666 | 1287 | GAGGTGCAGCTGTTGGAGTCGGGCCCAGGACTGGTGAAGCC | 7181 | EVQLLESGPGLVKP | 7532 | 6 | 6 |
| | | TTCGGAGACCCTGTCCCTCACCTGCACTGTCTCTGGCGGCT | | SETLSLTCTVSGGS | | | |
| | | CCATCAGTAATTACTACTGGAGCTGGATCCGGCAGCCCCCA | | ISNYYWSWIRQPPG | | | |
| | | GGGAAGGGACTGGAGTGGATTGGGTATATCTTTTACGGTGG | | KGLEWIGYIFYGGS | | | |
| | | CAGCACCAACTACAACCCCTCTCTCAGGAGTCGACTCACCA | | TNYNPSLRSRLTIS | | | |
| | | TATCAGTAGACACGTCCAAGAACCAGTTCTCCCTGAGGCTG | | VDTSKNQFSLRLSS | | | |
| | | AGCTCTGTGACCGCGGCGGACACGGCCGTTTATTACTGTGC | | VTAADTAVYYCARD | | | |
| | | GAGAGATCTCGGGTATAGCAGTGGCTGGCCTGATGCTTTTG | | LGYSSGWPDAFDIW | | | |
| | | ATATCTGGGGCCAAGGGACCACGGTCACCGTCTCCTCA | | GQGTTVTVSS | | | |
| ADI-81809 | 1286 | GAGGTGCAGCTGTTGGAGTCTGGGGGAGGCGTGGTCCAGCC | 7182 | EVQLLESGGGVVQP | 7533 | 6 | 5 |
| | | TGGGAGGTCCCTGAGACTCTCCTGTGCAGCCTCTGGATTCA | | GRSLRLSCAASGFT | | | |
| | | CCTTCAGTAGCTATGCTATGCACTGGGTCCGCCAGGCTCCA | | FSSYAMHWVRQAPG | | | |
| | | GGCAAGGGGCTGGAGTGGGTGGCAGTTATATCATCTGATGG | | KGLEWVAVISSDGS | | | |
| | | AAGCATGAAATTCTACGCAGACTCCGTGAAGGGCCGATTCA | | MKFYADSVKGRFTI | | | |
| | | CCATCTCCAGAGACAATTCCAAGAACACGCTGTTTCTGCAA | | SRDNSKNTLFLQMN | | | |
| | | ATGAACAGCCTCAGACCTGAGGACACGGCTGTATATTACTG | | SLRPEDTAVYYCAR | | | |
| | | TGCGAGAGATGCCGAAAACCTGGTTACCGAGTTTGACTACT | | DAENLVTEFDYWGQ | | | |
| | | GGGGCCAGGGAACCCTGGTCACCGTCTCCTCA | | GTLVTVSS | | | |
| ADI-81539 | 1286 | CAGGTGCAGCTGGTGCAGTCTGGGGCTGAGGTGAAGAAGCC | 7183 | QVQLVQSGAEVKKP | 7534 | 4 | 3 |
| | | TGGGGCCTCAGTGAAGGTTTCCTGCAAGGCATCTGGATACA | | GASVKVSCKASGYT | | | |
| | | CCTTCACCACTTACTATATGCACTGGGTGCGACTGGCCCCT | | FTTYYMHWVRLAPG | | | |
| | | GGACAAGGGCTTGAGTGGATGGGAATAATCAACCCTAGTGG | | QGLEWMGIINPSGG | | | |
| | | TGGTGGCACAAGCTACGCACAGAAGTTCCAGGGCAGAGTCA | | GTSYAQKFQGRVTM | | | |
| | | CCATGACCAGGGACACGTCCACGAGCACAGTCTACATGGAG | | TRDTSTSTVYMELS | | | |
| | | CTGAGCAGCCTGAGATCTGAGGACACGGCCGTGTATTATTG | | SLRSEDTAVYYCVR | | | |
| | | TGTGAGAGAAGGTTGTAGTAGTACCAGCTGCTTTGACTACT | | EGCSSTSCFDYWGQ | | | |
| | | GGGGCCAGGGAACCCTGGTCACCGTCTCCTCA | | GTLVTVSS | | | |
| ADI-81648 | 1291 | GAGGTGCAGCTGGTGGAGTCTGGGGGAGGCGTGGTCCAGCC | 7184 | EVQLVESGGGVVQP | 7535 | 0 | 0 |
| | | TGGGAGGTCCCTGAGACTCTCCTGTGCAGCGTCTGGATTCA | | GRSLRLSCAASGFT | | | |
| | | CCTTCAGTAGCTATGGCATGCACTGGGTCCGCCAGGCTCCA | | FSSYGMHWVRQAPG | | | |
| | | GGCAAGGGGCTGGAGTGGGTGGCAGTTATATGGTATGATGG | | KGLEWVAVIWYDGS | | | |
| | | AAGTAATAAATACTATGCAGACTCCGTGAAGGGCCGATTCA | | NKYYADSVKGRFTI | | | |
| | | CCATCTCCAGAGACAATTCCAAGAACACGCTGTATCTGCAA | | SRDNSKNTLYLQMN | | | |
| | | ATGAACAGCCTGAGAGCCGAGGACACGGCTGTGTATTACTG | | SLRAEDTAVYYCAR | | | |
| | | TGCGAGAGACAAAATCATTTGGGAGAGTGGTTATCACAAAA | | DKIIWESGYHKNYY | | | |
| | | ATTACTACTACTACTACTACATGGACGTCTGGGGCAAAGGG | | YYYYMDVWGKGTTV | | | |
| | | ACCACGGTCACCGTCTCCTCA | | TVSS | | | |

TABLE 6

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81604 | IGKV1D-39 | DTLMKQ | 3528 | AASKSS | 3709 | WYQQKP | 3906 | GASSRA | 4024 | GVPDRF | 4153 | QQSDSS | 4326 | FGRGTK |
| | | SPSSLL | | GSYLT | | GQAPRV | | S | | SGSGSG | | PFT | | VEIK |
| | | ASVGDA | | | | LIY | | | | TDFTLT | | | | |
| | | VISSS | | | | | | | | ISRLQP | | | | |
| | | | | | | | | | | EDFATY | | | | |
| | | | | | | | | | | FC | | | | |
| ADI-81710 | IGLV3-21 | SYELTQ | 3529 | GGNNIG | 3710 | WYQQKP | 3907 | DDSDRP | 2399 | GIPERF | 2545 | QVWDSN | 4327 | FGGGTK |
| | | PPSVSV | | SKGVH | | GQAPVL | | S | | SGSNSG | | SDLVV | | LTVL |
| | | APGQTA | | | | VVYDDS | | | | NTATLT | | | | |
| | | SITC | | | | DRPSGV | | | | ISRVEA | | | | |
| | | | | | | Y | | | | GDEADY | | | | |
| | | | | | | | | | | YC | | | | |
| ADI-81579 | IGKV1D-39 | DIQMTQ | 1878 | RSSQYV | 3711 | WYQLKP | 3908 | SASSLQ | 4025 | GVPSRF | 4154 | QQTFGH | 4328 | FGQGTK |
| | | SPSSLS | | SNFLN | | GKAPML | | D | | SGGGSG | | PWT | | VEIK |
| | | ASVGDR | | | | LIY | | | | TDFTLT | | | | |
| | | VTITC | | | | | | | | ITNVEP | | | | |
| | | | | | | | | | | ADFATY | | | | |
| | | | | | | | | | | SC | | | | |
| ADI-81656 | IGKV1D-39 | DITVTQ | 3530 | RANESI | 3712 | WYQLKP | 3909 | AASSLP | 4026 | GVPSRF | 2567 | QQSYSF | 4329 | FGQGTK |
| | | SPSSLS | | STFLS | | GKAPKL | | STLQS | | SGSGSG | | PWT | | VEIK |
| | | TSVGDR | | | | LIY | | | | TDFTLT | | | | |
| | | VTITC | | | | | | | | ISSLQP | | | | |
| | | | | | | | | | | EDFATY | | | | |
| | | | | | | | | | | FC | | | | |
| ADI-81640 | IGLV7-46 | PSEVTQ | 3531 | GSSTGP | 3713 | WFQQRP | 3910 | DTNYRQ | 4027 | WTPARF | 4155 | LLDYTY | 4330 | FGGGTK |
| | | EPSLTV | | VTTGHW | | GQAPRT | | S | | SGSLVG | | FRI | | LTVL |
| | | SPGGTV | | PF | | LFC | | | | GKAALT | | | | |
| | | TLTC | | | | | | | | LSGAQP | | | | |
| | | | | | | | | | | EDEAVY | | | | |
| | | | | | | | | | | YC | | | | |
| ADI-81586 | IGKV1-5 | DIQMTQ | 3532 | RASQSI | 3714 | WYQQKP | 2371 | DVSSLK | 4028 | GVPSRF | 4156 | QQYNSY | 4331 | FGQGTK |
| | | TPSTLS | | DTWLA | | GKAPEL | | RGER | | SGSGSG | | PWT | | VEIK |
| | | ASVGDR | | | | LIY | | | | TEFTLT | | | | |
| | | VTITC | | | | | | | | ISRLQP | | | | |
| | | | | | | | | | | DDLATY | | | | |
| | | | | | | | | | | YC | | | | |
| ADI-81663 | IGKV1-13 | ETTIKQ | 3533 | RASQSI | 3715 | WYQQKP | 3911 | DVSTLQ | 4029 | GVPSRF | 4157 | QHYHSY | 4332 | FGGGTK |
| | | SPSSLS | | SRFLK | | GKAHKL | | S | | SGSGCG | | PLT | | VEIK |
| | | ALVGDR | | | | MIH | | | | TDFTLT | | | | |
| | | VTITC | | | | | | | | ISSLQP | | | | |
| | | | | | | | | | | EDFATY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81604 | 2941 | GACACACTGATGAAGCAGTCTCCATCCTCCTTGTTGGCATCAG | 4606 | DTLMKQSPSSLLASV | 4960 | 33 | 20 |
| | | TAGGAGACGCCGTCATCTCCAGCAGCGCCGCAAGTAAGAGCAG | | GDAVISSSAASKSSG | | | |
| | | CGGCAGCTATTTAACTTGGTATCAGCAAAAACCAGGCCAAGCC | | SYLTWYQQKPGQAPR | | | |
| | | CCCAGGGTCCTGATCTATGGTGCATCCAGCAGGGCCAGTGGGG | | VLIYGASSRASGVPD | | | |
| | | TCCCAGACAGGTTCAGTGGCAGTGGATCTGGGACAGATTTCAC | | RFSGSGSGTDFTLTI | | | |
| | | TCTCACCATCAGCAGACTGCAACCTGAAGATTTTGCAACGTAT | | SRLQPEDFATYFCQQ | | | |
| | | TTCTGTCAACAGAGTGACAGTTCCCCGTTCACTTTCGGCCGGG | | SDSSPFTFGRGTKVE | | | |
| | | GGACCAAGGTGGAGATCAAA | | IK | | | |
| ADI-81710 | 2939 | TCGTATGAGCTGACACAGCCACCCTCGGTGTCAGTGGCCCCAG | 4607 | SYELTQPPSVSVAPG | 4961 | 32 | 12 |
| | | GACAGACGGCCAGCATTACCTGTGGGGGAAACAACATTGGAAG | | QTASITCGGNNIGSK | | | |
| | | TAAAGGTGTTCACTGGTACCAGCAGAAGCCAGGCCAGGCCCCT | | GVHWYQQKPGQAPVL | | | |
| | | GTGTTGGTCGTCTATGATGATAGCGACCGGCCCTCAGGGGTCT | | VVYDDSDRPSGVYDD | | | |
| | | ATGATGATAGCGACCGGCCCTCAGGGATCCCTGAGCGATTCTC | | SDRPSGIPERFSGSN | | | |
| | | TGGCTCCAACTCTGGGAACACGGCCACCCTGACCATCAGCAGG | | SGNTATLTISRVEAG | | | |
| | | GTCGAAGCCGGGGATGAGGCCGACTATTACTGTCAGGTGTGGG | | DEADYYCQVWDSNSD | | | |
| | | ATAGTAATAGTGATCTTGTGGTATTCGGCGGGGGGACCAAGCT | | LWVFGGGTKLTVL | | | |
| | | CACCGTCCTA | | | | | |
| ADI-81579 | 2937 | GACATCCAGATGACCCAGTCTCCGTCCTCTCTGTCCGCATCTG | 4608 | DIQMTQSPSSLSASV | 4962 | 23 | 16 |
| | | TAGGAGACAGAGTCACCATCACTTGTCGATCAAGTCAGTATGT | | GDRVTITCRSSQYVS | | | |
| | | CAGTAATTTTTTAAATTGGTATCAACTAAAACCAGGGAAAGCC | | NFLNWYQLKPGKAPM | | | |
| | | CCGATGCTCCTGATCTACTCTGCATCCAGTTTACAAGATGGGG | | LLIYSASSLQDGVPS | | | |
| | | TCCCATCCAGGTTCAGTGGCGGTGGATCTGGGACAGATTTCAC | | RFSGGGSGTDFTLTI | | | |
| | | TCTCACCATCACCAATGTTGAACCTGCAGATTTTGCGACCTAC | | TNVEPADFATYSCQQ | | | |
| | | TCCTGTCAGCAGACTTTTGGTCACCCGTGGACCTTCGGCCAAG | | TFGHPWTFGQGTKVE | | | |
| | | GGACCAAGGTGGAAATCAAA | | IK | | | |
| ADI-81656 | 2937 | GACATCACAGTGACCCAGTCTCCATCGTCCCTGTCTACATCTG | 4609 | DITVTQSPSSLSTSV | 4963 | 20 | 12 |
| | | TGGGAGACAGAGTCACCATCACTTGCCGGGCAAATGAGAGCAT | | GDRVTITCRANESIS | | | |
| | | TAGTACCTTTTTAAGTTGGTATCAGCTGAAACCAGGGAAAGCC | | TFLSWYQLKPGKAPK | | | |
| | | CCTAAGCTCCTGATCTATGCTGCATCCAGTCTACCCTCCACTT | | LLIYAASSLPSTLQS | | | |
| | | TACAAAGTGGGGTCCCATCAAGGTTCAGTGGCAGTGGATCTGG | | GVPSRFSGSGSGTDF | | | |
| | | GACAGATTTCACTCTCACCATCAGCAGTCTGCAACCTGAAGAT | | TLTISSLOPEDFATY | | | |
| | | TTTGCAACCTACTTCTGTCAACAAAGTTACAGTTTCCCGTGGA | | FCQQSYSFPWTFGQG | | | |
| | | CGTTCGGCCAAGGGACCAAGGTGGAAATCAAA | | TKVEIK | | | |
| ADI-81640 | 2939 | CCGTCTGAGGTGACTCAGGAGCCCTCACTGACTGTGTCCCCAG | 4610 | PSEVTQEPSLTVSPG | 4964 | 19 | 13 |
| | | GAGGGACAGTCACTCTCACCTGTGGCTCCAGCACTGGACCTGT | | GTVTLTCGSSTGPVT | | | |
| | | CACCACTGGTCATTGGCCCTTTTGGTTCCAGCAGAGGCCTGGC | | TGHWPFWFQQRPGQA | | | |
| | | CAAGCCCCCAGGACATTATTTTGTGATACAAATTACAGGCAGT | | PRTLFCDTNYRQSWT | | | |
| | | CTTGGACCCCTGCCAGGTTTTCAGGCTCCCTCGTCGGGGGCAA | | PARFSGSLVGGKAAL | | | |
| | | GGCTGCCCTGACCCTTTCGGGTGCGCAGCCTGAAGATGAGGCT | | TLSGAQPEDEAVYYC | | | |
| | | GTCTATTATTGCTTACTCGACTATACTTATTTTCGGATATTCG | | LLDYTYFRIFGGGTK | | | |
| | | GCGGAGGGACCAAGCTGACCGTCCTA | | LTVL | | | |
| ADI-81586 | 2937 | GACATCCAGATGACCCAGACTCCCTCCACCCTGTCTGCATCTG | 4611 | DIQMTQTPSTLSASV | 4965 | 18 | 10 |
| | | TGGGAGACAGAGTCACAATCACTTGCCGGGCCAGTCAGAGTAT | | GDRVTITCRASQSID | | | |
| | | CGATACCTGGTTGGCCTGGTATCAGCAAAAACCAGGGAAAGCC | | TWLAWYQQKPGKAPE | | | |
| | | CCTGAACTCCTGATTTATGATGTCTCCAGTTTGAAAAGAGGGG | | LLIYDVSSLKRGERG | | | |
| | | AAAGAGGGGTCCCATCAAGGTTCAGCGGCAGTGGATCTGGGAC | | VPSRFSGSGSGTEFT | | | |
| | | AGAATTCACTCTCACCATCAGCAGACTGCAGCCTGATGATCTT | | LTISRLQPDDLATYY | | | |
| | | GCAACTTATTATTGCCAACAGTATAATAGTTACCCTTGGACGT | | CQQYNSYPWTFGQGT | | | |
| | | TCGGCCAAGGGACCAAGGTGGAAATCAAA | | KVEIK | | | |
| ADI-81663 | 2936 | GAAACGACAATCAAGCAGTCTCCATCCTCCTTGTCTGCATTAG | 4612 | ETTIKQSPSSLSALV | 4966 | 18 | 12 |
| | | TAGGAGACAGAGTCACCATCACTTGCCGGGCAAGTCAGAGTAT | | GDRVTITCRASQSIS | | | |
| | | TAGTAGGTTTTTAAAGTGGTATCAGCAGAAACCAGGGAAAGCC | | RFLKWYQQKPGKAHK | | | |
| | | CATAAACTCATGATCCATGATGTTTCCACTTTGCAAAGTGGGG | | LMIHDVSTLQSGVPS | | | |
| | | TCCCATCAAGGTTCAGCGGCAGTGGATGTGGGACAGATTTCAC | | RFSGSGCGTDFTLTI | | | |
| | | TCTCACCATCAGCAGCCTGCAGCCTGAAGATTTTGCAACTTAT | | SSLQPEDFATYYCQH | | | |
| | | TATTGCCAACACTATCATAGTTACCCTCTCACTTTCGGCGGAG | | YHSYPLTFGGGTKVE | | | |
| | | GGACCAAGGTGGAGATCAAA | | IK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81829 | IGKV3-20 | EMTLTQ | 3534 | RASQ- | 3716 | WYQQKP | 3912 | GASTRA | 4030 | GIPDRF | 4158 | HQYSDS | 4333 | FGGGTK |
| | | SPATLS | | NINIYL | | GQAPRL | | S | | SGSGSG | | PPLT | | VEIK |
| | | LSPGER | | A | | LIF | | | | TDFTLT | | | | |
| | | ATLSC | | | | | | | | VRRLEP | | | | |
| | | | | | | | | | | EDFAVY | | | | |
| | | | | | | | | | | FC | | | | |
| ADI-81786 | IGKV1-33 | EMAMTQ | 3535 | QASQDI | 3717 | WYQQKP | 3913 | TAANLE | 4031 | GVSSRF | 4159 | QQYNDF | 4334 | FGQGTK |
| | | SPSSMS | | TDYLN | | GSAPKL | | A | | SGSGSG | | PYT | | VEIK |
| | | ASVGDR | | | | LIY | | | | THFTFT | | | | |
| | | VTITC | | | | | | | | ISGLQP | | | | |
| | | | | | | | | | | EDVATY | | | | |
| | | | | | | | | | | YC | | | | |
| ADI-81651 | IGLV2-14 | QSALTQ | 3536 | TGASGD | 3718 | WYQQEP | 3914 | GVSNRP | 4032 | GVSDRF | 4160 | SSYTRS | 4335 | FGGGTK |
| | | PASVSG | | IDTYNY | | GKAPKL | | S | | SGSKSA | | NTVK | | ILTVL |
| | | SAGQSI | | VS | | MIY | | | | NTASLT | | | | |
| | | TISC | | | | | | | | ISGLQP | | | | |
| | | | | | | | | | | EDEADY | | | | |
| | | | | | | | | | | YC | | | | |
| ADI-81725 | IGLV1-40 | QSVLTQ | 3537 | TGSTSN | 2149 | WYQQLP | 2317 | LNNNRP | 2454 | GVPDRF | 2601 | QSYDSS | 2800 | FGGGTK |
| | | PPSVSG | | IGGGYD | | GEAPKL | | S | | SGSKSG | | LSVWV | | LTVL |
| | | APGRGV | | VH | | LIY | | | | TSASLA | | | | |
| | | TISC | | | | | | | | ITGLQP | | | | |
| | | | | | | | | | | VDEADY | | | | |
| | | | | | | | | | | YC | | | | |
| ADI-81706 | IGKV1D-39 | EIQMTQ | 1881 | RASQDI | 3719 | WYQQKP | 3915 | AASTLQ | 4033 | GVPSRF | 4161 | QQTYST | 4336 | FGQGTK |
| | | SPSSLS | | STYVN | | GGAPKL | | T | | TGSGSG | | HMST | | VEIK |
| | | ASVGDR | | | | LIY | | | | THFTLT | | | | |
| | | VTITC | | | | | | | | ITSLQP | | | | |
| | | | | | | | | | | EDFATY | | | | |
| | | | | | | | | | | SC | | | | |
| ADI-81881 | IGLV1-40 | QSVLTQ | 1948 | TGSSSN | 2250 | WYQVLP | 3916 | GYTKRP | 4034 | GVPARF | 4162 | QSYDNS | 4337 | FGGGTK |
| | | PPSVSG | | IGTGYD | | GSAPKL | | S | | SGSRSG | | LSASV | | LTVL |
| | | APGQRV | | VH | | LIY | | | | SSASLV | | | | |
| | | TISC | | | | | | | | ITGLQA | | | | |
| | | | | | | | | | | EDEADY | | | | |
| | | | | | | | | | | YC | | | | |
| ADI-81607 | IGKV1-12 | DIQMTQ | 1878 | RASQDV | 3720 | WYQQKP | 2257 | SASFLY | 4035 | GVPSRF | 4163 | QQHYTT | 4338 | FGQGTK |
| | | SPSSLS | | NTAVA | | GKAPKL | | S | | SGSRSG | | PPT | | VEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81766 | IGKV3-20 | EIMLTQ | 3538 | GSGFLA | 3721 | WYQQKP | 2258 | AGSGRA | 4036 | GIPDRF | 4164 | QQYGSS | 2929 | FGQGTK |
| | | SPGTLS | | RARQSV | | GQAPRL | | P | | SGSGFG | | PYT | | VDIK |
| | | LSPGER | | | | LIY | | | | TDFTLT | | | | |
| | | ATLSR | | | | | | | | LSRLEP | | | | |
| | | | | | | | | | | EDFAVY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81829 | 2936 | GAAATGACATTGACGCAGTCTCCAGCCACCCTGTCTTTGTCTC | 4613 | EMTLTQSPATLSLSP | 4967 | 17 | 12 |
| | | CAGGGGAAAGAGCCACCCTCTCCTGCAGGGCCAGTCAGAATAT | | GERATLSCRASQNIN | | | |
| | | TAACATCTACTTAGCCTGGTACCAGCAGAAACCTGGCCAGGCT | | IYLAWYQQKPGQAPR | | | |
| | | CCCAGGCTCCTCATCTTTGGTGCTTCTACGAGGGCCAGTGGCA | | LLIFGASTRASGIPD | | | |
| | | TCCCAGACAGGTTCAGTGGCAGTGGGTCTGGGACAGACTTCAC | | RFSGSGSGTDFTLTV | | | |
| | | TCTCACCGTCAGGAGACTGGAGCCTGAAGATTTTGCAGTCTAT | | RRLEPEDFAVYFCHQ | | | |
| | | TTCTGCCATCAATACAGTGACTCACCTCCGCTCACTTTCGGCG | | YSDSPPLTFGGGTKV | | | |
| | | GAGGGACCAAGGTGGAGATCAAA | | EIK | | | |
| ADI-81786 | 2937 | GAAATGGCGATGACGCAGTCTCCATCCTCCATGTCTGCATCTG | 4614 | EMAMTQSPSSMSASV | 4968 | 15 | 11 |
| | | TGGGAGACAGAGTCACCATCACCTGCCAGGCGAGTCAGGACAT | | GDRVTITCQASQDIT | | | |
| | | TACCGACTATTTAAATTGGTATCAGCAGAAACCAGGGAGTGCC | | DYLNWYQQKPGSAPK | | | |
| | | CCTAAACTCCTGATCTACACTGCAGCCAATTTGGAAGCCGGGG | | LLIYTAANLEAGVSS | | | |
| | | TCTCGTCCAGGTTCAGTGGAAGTGGATCTGGGACACATTTTAC | | RFSGSGSGTHFTFTI | | | |
| | | TTTCACCATCAGCGGCCTCCAGCCTGAAGATGTTGCAACATAT | | SGLQPEDVATYYCQQ | | | |
| | | TACTGTCAACAGTATAATGATTTCCCATACACTTTTGGCCAGG | | YNDFPYTFGQGTKVE | | | |
| | | GGACCAAGGTGGAAATCAAA | | IK | | | |
| ADI-81651 | 2939 | CAGTCTGCCCTGACTCAGCCTGCCTCCGTGTCTGGGTCTGCTG | 4615 | QSALTQPASVSGSAG | 4969 | 14 | 11 |
| | | GACAGTCGATCACCATCTCCTGCACTGGAGCCAGCGGTGACAT | | QSITISCTGASGDID | | | |
| | | TGATACTTATAACTATGTCTCCTGGTACCAACAGGAACCAGGC | | TYNYVSWYQQEPGKA | | | |
| | | AAAGCCCCCAAACTCATGATTTATGGTGTCAGTAATCGGCCCT | | PKLMIYGVSNRPSGV | | | |
| | | CAGGGGTTTCTGATCGCTTCTCTGGCTCCAAGTCTGCCAACAC | | SDRFSGSKSANTASL | | | |
| | | GGCCTCCCTGACCATCTCTGGGCTCCAGCCTGAGGACGAGGCT | | TISGLQPEDEADYYC | | | |
| | | GATTATTACTGCAGCTCATATACAAGGAGTAACACTGTGAAAT | | SSYTRSNTVKFGGGT | | | |
| | | TCGGCGGCGGGACCAAGCTCACCGTCCTA | | KLTVL | | | |
| ADI-81725 | 2939 | CAGTCTGTCCTGACGCAGCCGCCCTCAGTGTCTGGGGCCCCAG | 4616 | QSVLTQPPSVSGAPG | 4970 | 14 | 9 |
| | | GGCGGGGGGTCACCATCTCCTGCACTGGGAGCACCTCCAACAT | | RGVTISCTGSTSNIG | | | |
| | | CGGGGGAGGTTATGATGTGCACTGGTACCAGCAGCTTCCAGGA | | GGYDVHWYQQLPGEA | | | |
| | | GAGGCCCCCAAACTCCTCATCTATCTTAACAATAATCGGCCCT | | PKLLIYLNNNRPSGV | | | |
| | | CAGGGGTCCCTGACCGATTCTCTGGCTCCAAGTCTGGCACCTC | | PDRFSGSKSGTSASL | | | |
| | | AGCCTCCCTGGCCATCACTGGGCTCCAGCCTGTCGATGAGGCT | | AITGLQPVDEADYYC | | | |
| | | GATTATTACTGCCAGTCGTATGACAGCAGCCTGAGTGTTTGGG | | QSYDSSLSVWVFGGG | | | |
| | | TGTTCGGCGGAGGGACCAAGCTCACCGTCCTA | | TKLTVL | | | |
| ADI-81706 | 2937 | GAAATCCAGATGACCCAGTCTCCATCCTCCCTGTCTGCATCTG | 4617 | EIQMTQSPSSLSASV | 4971 | 14 | 10 |
| | | TAGGAGACAGAGTCACCATCACTTGCCGGGCAAGTCAAGACAT | | GDRVTITCRASQDIS | | | |
| | | TAGTACTTATGTAAATTGGTATCAGCAGAAACCTGGGGGGGCC | | TYVNWYQQKPGGAPK | | | |
| | | CCTAAACTCCTGATCTATGCTGCCTCCACTTTGCAAACTGGGG | | LLIYAASTLQTGVPS | | | |
| | | TCCCATCAAGGTTTACTGGCAGTGGATCTGGGACACATTTCAC | | RFTGSGSGTHFTLTI | | | |
| | | TCTCACCATCACCAGTCTGCAACCTGAAGATTTTGCAACTTAC | | TSLQPEDFATYSCQQ | | | |
| | | TCCTGTCAACAGACTTACAGTACCCACATGTCCACTTTTGGCC | | TYSTHMSTFGQGTKV | | | |
| | | AGGGGACCAAGGTGGAAATCAAA | | EIK | | | |
| ADI-81881 | 2939 | CAGTCTGTGTTGACGCAGCCGCCCTCAGTGTCTGGGGCCCCAG | 4618 | QSVLTQPPSVSGAPG | 4972 | 14 | 10 |
| | | GGCAGAGGGTCACCATCTCCTGCACTGGGAGCAGCTCCAACAT | | QRVTISCTGSSSNIG | | | |
| | | CGGGACAGGTTATGATGTACACTGGTACCAGGTTCTTCCAGGA | | TGYDVHWYQVLPGSA | | | |
| | | TCCGCCCCCAAACTCCTCATCTATGGTTACACAAAACGGCCCT | | PKLLIYGYTKRPSGV | | | |
| | | CAGGGGTCCCTGCCCGATTCTCTGGCTCCAGGTCTGGCAGCTC | | PARFSGSRSGSSASL | | | |
| | | AGCCTCCCTGGTCATCACTGGGCTCCAGGCTGAGGATGAGGCT | | VITGLQAEDEADYYC | | | |
| | | GATTATTACTGCCAGTCCTATGACAACAGCCTGAGTGCTTCGG | | QSYDNSLSASVFGGG | | | |
| | | TGTTCGGCGGAGGGACCAAGCTCACCGTCCTA | | TKLTVL | | | |
| ADI-81607 | 2937 | GACATCCAAATGACCCAGTCTCCATCTTCCCTGTCTGCATCTG | 4619 | DIQMTQSPSSLSASV | 4973 | 14 | 11 |
| | | TAGGAGACAGAGTCACCATCACTTGTCGGGCGAGTCAGGATGT | | GDRVTITCRASQDVN | | | |
| | | TAACACCGCGGTAGCCTGGTATCAGCAGAAACCAGGGAAAGCC | | TAVAWYQQKPGKAPK | | | |
| | | CCTAAGCTCCTGATCTATTCTGCATCCTTTTTGTACAGTGGGG | | LLIYSASFLYSGVPS | | | |
| | | TCCCATCAAGGTTCAGCGGCAGTAGATCTGGGACAGATTTCAC | | RFSGSRSGTDFTLTI | | | |
| | | TCTCACCATCAGCAGCCTGCAGCCTGAAGATTTTGCAACTTAC | | SSLQPEDFATYYCQQ | | | |
| | | TATTGTCAGCAACACTACACTACGCCACCCACGTTCGGCCAAG | | HYTTPPTFGQGTKVE | | | |
| | | GGACCAAGGTGGAAATCAAA | | IK | | | |
| ADI-81766 | 2944 | GAAATTATGCTGACCCAGTCTCCAGGCACCCTGTCTTTGTCTC | 4620 | EIMLTQSPGTLSLSP | 4974 | 13 | 11 |
| | | CAGGGGAAAGAGCCACCCTCTCCCGCAGGGCCCGTCAGAGTGT | | GERATLSRRARQSVG | | | |
| | | TGGCAGCGGCTTTTTAGCCTGGTACCAGCAGAAACCCGGCCAG | | SGFLAWYQQKPGQAP | | | |
| | | GCTCCCAGGCTCCTCATTTATGCTGGGTCCGGCAGGGCCCCTG | | RLLIYAGSGRAPGIP | | | |
| | | GCATCCCAGACAGGTTCAGTGGCAGTGGCTTTGGGACAGACTT | | DRFSGSGFGTDFTLT | | | |
| | | CACTCTCACCCTCAGCAGACTGGAGCCTGAAGATTTTGCAGTG | | LSRLEPEDFAVYYCQ | | | |
| | | TATTACTGTCAGCAGTATGGTAGCTCACCGTACACTTTTGGCC | | QYGSSPYTFGQGTKV | | | |
| | | AGGGGACCAAAGTGGATATCAAA | | DIK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81687 | IGKV1D-39 | DIRLTQ | 3539 | RASQNI | 3722 | WYQKKP | 3917 | AASTLQ | 2392 | VVPPRF | 4165 | QQSYNT | 4339 | FGQGTR |
| | | SPSSLS | | ATFLN | | GKALKL | | S | | SGSGSG | | LSIT | | LEIK |
| | | ASVGDR | | | | LIY | | | | TDFTLT | | | | |
| | | VTITC | | | | | | | | ISSLQP | | | | |
| | | | | | | | | | | DDFATY | | | | |
| | | | | | | | | | | YC | | | | |
| ADI-81908 | IGKV1D-39 | DIRLTQ | 3539 | RASQTV | 3723 | WYQQRP | 2309 | AASTLE | 4037 | GVPSRF | 4166 | QQSYST | 4340 | FGGGTK |
| | | SPSSLS | | NSYLN | | GKAPKL | | T | | TGSGSG | | LELT | | VDIK |
| | | ASVGDR | | | | LIY | | | | TDFTLT | | | | |
| | | VTITC | | | | | | | | ISGLQP | | | | |
| | | | | | | | | | | EDFATY | | | | |
| | | | | | | | | | | YC | | | | |
| ADI-81827 | IGLV3-21 | SYELTQ | 3529 | GGNNIA | 3724 | WYQQKP | 2287 | DDRERP | 4038 | GIPERF | 4167 | QVWDRS | 4341 | FGTGTK |
| | | PPSVSV | | DQIVH | | GQAPVL | | S | | SGSHSG | | GHHPVF | | VTVL |
| | | APGQTA | | | | VVF | | | | NTATLT | | V | | |
| | | SITC | | | | | | | | ISRVEA | | | | |
| | | | | | | | | | | GDEGDY | | | | |
| | | | | | | | | | | YC | | | | |
| ADI-81875 | IGKV3-11 | EIPMTQ | 3540 | RASQSV | 3725 | WYQQRP | 3918 | DASHRA | 4039 | DIPARF | 4168 | HQRRNW | 4342 | FGQGTK |
| | | SPATLS | | GTYLG | | GQAPRL | | T | | SGSGSG | | PPT | | VEIK |
| | | LAPGDR | | | | LIF | | | | TNFTLT | | | | |
| | | ATLSC | | | | | | | | ISSLEP | | | | |
| | | | | | | | | | | EDFVVY | | | | |
| | | | | | | | | | | YC | | | | |
| ADI-81885 | IGLV2-14 | SLFLTQ | 3541 | TGASSD | 3726 | WYQQHP | 3919 | EVFNRP | 4040 | GVSSRF | 4169 | CSYTSR | 4343 | FGTGTK |
| | | PASVSG | | VGGYNF | | GKTPKL | | S | | SGSKSG | | STYV | | VTVL |
| | | SPGQSI | | VS | | ILY | | | | STASLT | | | | |
| | | TISC | | | | | | | | ISGLQD | | | | |
| | | | | | | | | | | EDEADY | | | | |
| | | | | | | | | | | YC | | | | |
| ADI-81638 | IGKV1-5 | EIPMTQ | 3542 | RASQSI | 3727 | WYQQKP | 2371 | KASSIE | 4041 | GVPSRF | 4170 | QQYSAY | 4344 | FGQGTK |
| | | SPSTLS | | GPWLA | | GKAPEL | | T | | SGSWSG | | PYT | | VEIK |
| | | ASVGDR | | | | LIY | | | | TEFTLT | | | | |
| | | VTITC | | | | | | | | ISSLQA | | | | |
| | | | | | | | | | | SDFATY | | | | |
| | | | | | | | | | | YC | | | | |
| ADI-81554 | IGKV1D-39 | EIQMTQ | 3543 | RASQSI | 3728 | WYQQKP | 2316 | AASYLQ | 4042 | GVPSRF | 4171 | QQSYTT | 4345 | FGGGTK |
| | | SPPSLS | | SRYLH | | GKAPKV | | S | | SGSGSG | | PLT | | VEIK |
| | | ASVGDS | | | | LIY | | | | TDFSLT | | | | |
| | | VTITC | | | | | | | | ISGLQP | | | | |
| | | | | | | | | | | EDLATY | | | | |
| | | | | | | | | | | YC | | | | |
| ADI-81756 | IGKV1D-39 | EMTVTQ | 3544 | RASQYI | 3729 | WYQQKP | 2257 | AASTLQ | 4033 | GVPSRF | 4172 | QQSYNT | 4346 | FGGGTK |
| | | SPSSLS | | NNYLN | | GKAPKL | | T | | SGSGSG | | PPLT | | VEIK |
| | | ASVGDR | | | | LIY | | | | TDFSLT | | | | |
| | | VTISC | | | | | | | | ISSLHP | | | | |
| | | | | | | | | | | EDFATY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81687 | 2940 | GACATCCGGTTGACCCAGTCTCCATCCTCCCTGTCTGCATCTG | 4621 | DIRLTQSPSSLSASV | 4975 | 12 | 10 |
| | | TGGGAGACAGAGTCACCATCACTTGCCGGGCAAGTCAGAACAT | | GDRVTITCRASQNIA | | | |
| | | TGCCACCTTTTTAAATTGGTATCAAAAGAAACCAGGGAAAGCC | | TFLNWYQKKPGKALK | | | |
| | | CTTAAACTCTTGATTTATGCTGCATCCACCTTGCAAAGTGTGG | | LLIYAASTLQSVVPP | | | |
| | | TCCCGCCAAGGTTCAGTGGCAGTGGATCTGGGACAGATTTCAC | | RFSGSGSGTDFTLTI | | | |
| | | TCTCACCATCAGCAGTCTGCAACCTGACGATTTTGCAACTTAC | | SSLQPDDFATYYCQQ | | | |
| | | TACTGTCAACAGAGTTACAATACCCTCTCTATCACCTTCGGCC | | SYNTLSITFGQGTRL | | | |
| | | AAGGGACACGACTGGAGATTAAA | | EIK | | | |
| ADI-81908 | 2938 | GACATCCGGTTGACCCAGTCTCCATCCTCCCTGTCTGCATCTG | 4622 | DIRLTQSPSSLSASV | 4976 | 11 | 9 |
| | | TAGGAGACAGAGTCACCATCACTTGCCGGGCAAGTCAGACTGT | | GDRVTITCRASQTVN | | | |
| | | TAACTCCTATTTAAATTGGTATCAGCAGAGACCAGGTAAAGCC | | SYLNWYQQRPGKAPK | | | |
| | | CCTAAGCTCCTGATCTATGCTGCTTCCACTTTAGAAACTGGGG | | LLIYAASTLETGVPS | | | |
| | | TCCCATCAAGGTTCACCGGCAGTGGATCTGGGACAGATTTCAC | | RFTGSGSGTDFTLTI | | | |
| | | TCTCACCATCAGCGGTCTGCAACCTGAGGATTTTGCAACTTAC | | SGLQPEDFATYYCQQ | | | |
| | | TACTGTCAACAGAGTTACAGTACCCTTGAGCTCACTTTCGGCG | | SYSTLELTFGGGTKV | | | |
| | | GAGGGACCAAGGTGGATATCAAA | | DIK | | | |
| ADI-81827 | 2945 | TCGTATGAGCTGACTCAGCCACCCTCGGTGTCAGTGGCCCCCG | 4623 | SYELTQPPSVSVAPG | 4977 | 11 | 10 |
| | | GACAGACGGCCAGCATTACCTGTGGGGGAAACAATATTGCAGA | | QTASITCGGNNIADQ | | | |
| | | TCAAATTGTGCACTGGTACCAGCAGAAGCCAGGCCAGGCCCCT | | IVHWYQQKPGQAPVL | | | |
| | | GTTTTGGTCGTCTTTGATGATCGCGAGCGGCCCTCAGGGATCC | | VVFDDRERPSGIPER | | | |
| | | CTGAGCGATTCTCTGGCTCCCACTCTGGGAACACGGCCACCCT | | FSGSHSGNTATLTIS | | | |
| | | GACCATCAGCAGGGTCGAAGCCGGGGACGAGGGCGACTATTAT | | RVEAGDEGDYYCQVW | | | |
| | | TGTCAGGTGTGGGATCGGAGTGGTCATCATCCAGTTTTTGTCT | | DRSGHHPVFVFGTGT | | | |
| | | TCGGCACTGGCACCAAGGTCACCGTCCTA | | KVTVL | | | |
| ADI-81875 | 2937 | GAAATCCCAATGACCCAGTCTCCAGCCACCCTGTCTTTGGCTC | 4624 | EIPMTQSPATLSLAP | 4978 | 11 | 11 |
| | | CAGGGGACAGGGCCACCCTCTCCTGCAGGGCCAGTCAGAGTGT | | GDRATLSCRASQSVG | | | |
| | | TGGCACCTACTTAGGCTGGTACCAACAAAGACCTGGCCAGGCT | | TYLGWYQQRPGQAPR | | | |
| | | CCCAGGCTCCTCATCTTTGATGCATCCCACAGGGCCACTGACA | | LLIFDASHRATDIPA | | | |
| | | TCCCAGCCAGGTTCAGTGGCAGTGGGTCTGGGACAAACTTCAC | | RFSGSGSGTNFTLTI | | | |
| | | TCTCACCATCAGCAGCCTAGAGCCTGAAGACTTTGTAGTTTAT | | SSLEPEDFVVYYCHQ | | | |
| | | TACTGTCACCAGCGTCGCAACTGGCCTCCGACTTTCGGCCAGG | | RRNWPPTFGQGTKVE | | | |
| | | GGACCAAGGTGGAGATCAAA | | IK | | | |
| ADI-81885 | 2945 | AGTCTGTTCCTGACTCAGCCTGCCTCCGTGTCTGGGTCTCCTG | 4625 | SLFLTQPASVSGSPG | 4979 | 11 | 9 |
| | | GACAGTCGATCACCATCTCCTGCACTGGAGCCAGCAGTGACGT | | QSITISCTGASSDVG | | | |
| | | TGGTGGTTATAACTTTGTTTCCTGGTACCAACAACACCCAGGG | | GYNFVSWYQQHPGKT | | | |
| | | AAAACCCCCAAACTCATACTTTATGAGGTCTTCAATCGCCCCT | | PKLILYEVFNRPSGV | | | |
| | | CAGGGGTGTCCAGTCGCTTCTCTGGCTCCAAGTCTGGCAGCAC | | SSRFSGSKSGSTASL | | | |
| | | GGCCTCCCTGACGATCTCTGGGCTCCAGGATGAGGACGAGGCT | | TISGLQDEDEADYYC | | | |
| | | GATTACTACTGCTGTTCATATACAAGCAGGAGCACTTATGTCT | | CSYTSRSTYVFGTGT | | | |
| | | TCGGAACTGGGACCAAGGTCACCGTCCTA | | KVTVL | | | |
| ADI-81638 | 2937 | GAAATCCCGATGACCCAGTCTCCTTCCACCCTGTCTGCATCTG | 4626 | EIPMTQSPSTLSASV | 4980 | 11 | 8 |
| | | TAGGAGACAGAGTCACCATCACTTGCCGGGCCAGTCAGAGTAT | | GDRVTITCRASQSIG | | | |
| | | TGGTCCCTGGTTGGCCTGGTATCAGCAGAAACCAGGGAAAGCC | | PWLAWYQQKPGKAPE | | | |
| | | CCTGAGCTCCTGATCTATAAGGCGTCTAGTATAGAAACTGGGG | | LLIYKASSIETGVPS | | | |
| | | TCCCATCAAGGTTCAGCGGCAGTTGGTCTGGGACAGAATTCAC | | RFSGSWSGTEFTLTI | | | |
| | | TCTCACCATCAGCAGCCTGCAGGCTTCTGATTTTGCAACTTAT | | SSLQASDFATYYCQQ | | | |
| | | TACTGCCAACAGTATAGCGCTTATCCGTACACTTTTGGCCAGG | | YSAYPYTFGQGTKVE | | | |
| | | GGACCAAGGTGGAGATCAAA | | IK | | | |
| ADI-81554 | 2936 | GAAATCCAGATGACCCAGTCTCCACCCTCCCTGTCTGCATCTG | 4627 | EIQMTQSPPSLSASV | 4981 | 11 | 9 |
| | | TAGGAGACAGCGTCACCATCACTTGCCGGGCAAGTCAGAGCAT | | GDSVTITCRASQSIS | | | |
| | | TAGTAGGTATTTACATTGGTATCAGCAGAAACCCGGGAAAGCC | | RYLHWYQQKPGKAPK | | | |
| | | CCTAAGGTTCTGATCTATGCTGCATCCTATTTGCAAAGTGGGG | | VLIYAASYLQSGVPS | | | |
| | | TCCCTTCAAGGTTCAGTGGCAGTGGATCTGGGACAGATTTCAG | | RFSGSGSGTDFSLTI | | | |
| | | TCTCACCATCAGCGGTCTGCAGCCTGAAGATCTTGCAACTTAC | | SGLQPEDLATYYCQQ | | | |
| | | TACTGTCAACAGAGTTACACTACCCCGCTCACTTTCGGCGGAG | | SYTTPLTFGGGTKVE | | | |
| | | GGACCAAGGTGGAAATCAAA | | IK | | | |
| ADI-81756 | 2936 | GAAATGACGGTGACGCAGTCTCCATCCTCCCTGTCTGCATCTG | 4628 | EMTVTQSPSSLSASV | 4982 | 11 | 8 |
| | | TAGGAGACAGAGTCACCATCAGTTGCCGGGCAAGCCAGTACAT | | GDRVTISCRASQYIN | | | |
| | | TAATAATTATTTAAATTGGTATCAGCAGAAACCAGGGAAAGCC | | NYLNWYQQKPGKAPK | | | |
| | | CCTAAGCTCCTGATCTATGCTGCATCCACTTTGCAAACTGGGG | | LLIYAASTLQTGVPS | | | |
| | | TCCCATCAAGGTTCAGTGGCAGTGGATCTGGGACAGATTTCAG | | RFSGSGSGTDFSLTI | | | |
| | | TCTCACCATCAGCAGTCTACACCCTGAAGATTTTGCAACTTAC | | SSLHPEDFATYYCQQ | | | |
| | | TACTGTCAGCAGAGTTACAATACCCCTCCGCTCACTTTCGGCG | | SYNTPPLTFGGGTKV | | | |
| | | GAGGGACCAAGGTGGAAATCAAA | | EIK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81773 | IGKV1D-39 | DIRMTQ | 1937 | RASQSI | 3730 | WYQQKT | 3920 | AASRLQ | 2406 | GVPSRF | 4173 | QQTYIM | 4347 | FGQGTK |
| | | SPSSLS | | GGYLN | | GRAPRL | | S | | SGSGSG | | PPWT | | VEIK |
| | | ASVGDR | | | | LIF | | | | TEFTLT | | | | |
| | | VTITC | | | | | | | | ISGLQP | | | | |
| | | | | | | | | | | DDFATY | | | | |
| | | | | | | | | | | YC | | | | |
| ADI-81575 | IGKV1D-39 | DIQMTQ | 1907 | RASQNI | 3731 | WYQQKP | 3921 | AAASLH | 4043 | GVPSRF | 4174 | QQSYST | 2715 | FGLGTR |
| | | SPSSLS | | STYLN | | GTAPNL | | S | | SGSGSG | | PIT | | LEIK |
| | | ASIGDR | | | | LIY | | | | TDFTLT | | | | |
| | | VTITC | | | | | | | | ISSLER | | | | |
| | | | | | | | | | | EDFATY | | | | |
| | | | | | | | | | | YC | | | | |
| ADI-81727 | IGKV2D-29 | DIVMTQ | 3545 | ESSKSL | 3732 | WYLQRP | 3922 | EVSKRF | 4044 | GVPDRF | 4175 | MQSIQL | 4348 | FGQGTK |
| | | SPLSLS | | LHSGDG | | GQPPQL | | A | | TGSGSG | | PPFT | | VEIK |
| | | VTPGQS | | KTYLY | | LIY | | | | TDFTLK | | | | |
| | | ASISC | | | | | | | | ISRVEA | | | | |
| | | | | | | | | | | EDVGIY | | | | |
| | | | | | | | | | | YC | | | | |
| ADI-81704 | IGKV3-20 | EITLTQ | 3546 | RASESV | 3733 | WHQQRP | 2342 | GASKRP | 4045 | GIPDRF | 2616 | HQFASA | 2848 | FGQGTR |
| | | SPGTLS | | SSEHLA | | GQAPRL | | T | | SGSGSG | | PIT | | LEIK |
| | | LSPGER | | | | LIY | | | | TDFTLT | | | | |
| | | ATLSC | | | | | | | | ISRLEP | | | | |
| | | | | | | | | | | EDFAVY | | | | |
| | | | | | | | | | | YC | | | | |
| ADI-81768 | IGLV2-8 | QPVMTQ | 3547 | TGTSSD | 3734 | WYQQHP | 3923 | EVNKRP | 2394 | GVPDRL | 4176 | SSNAGV | 4349 | FGGGTK |
| | | PPSASG | | VGDSNY | | GNPPKL | | S | | SGSKSG | | TALI | | VTVL |
| | | SPGQSV | | VS | | IIY | | | | NTASLT | | | | |
| | | TISC | | | | | | | | VSGLLP | | | | |
| | | | | | | | | | | EDEAHY | | | | |
| | | | | | | | | | | YC | | | | |
| ADI-81775 | IGKV1D-39 | EIVMTQ | 1891 | RASQTI | 3735 | WYQHKP | 3924 | GASTLL | 4046 | GVPSRF | 4177 | QQTYST | 4350 | FAQGTK |
| | | SPSSLS | | NSYLN | | GKAPKL | | S | | SGSESG | | PPWT | | VDIK |
| | | ASVGDR | | | | LIY | | | | TDYTLT | | | | |
| | | VTITC | | | | | | | | INGLQP | | | | |
| | | | | | | | | | | EDFATY | | | | |
| | | | | | | | | | | YC | | | | |
| ADI-81598 | IGKV1D-39 | DIQMTQ | 1878 | RTSLNI | 3736 | WYQQKP | 2257 | AASTLQ | 2392 | GVPSRF | 4178 | QQSYVT | 4351 | FGQGTK |
| | | SPSSLS | | KNYLN | | GKAPKL | | S | | RGSGSG | | PPWT | | VEIK |
| | | ASVGDR | | | | LIY | | | | TDFTLT | | | | |
| | | VTITC | | | | | | | | ISGLQP | | | | |
| | | | | | | | | | | EDFATY | | | | |
| | | | | | | | | | | FC | | | | |
| ADI-81914 | IGLV3-21 | QSVLTQ | 3548 | GGDNIG | 3737 | WYQRKP | 3925 | DDSDRP | 2399 | GIPERF | 4179 | QVWDTS | 4352 | FGAGTK |
| | | PPSVSL | | SYRVH | | GQAPIL | | S | | SGSTSG | | RSTHHR | | LTVL |
| | | APGQTA | | | | VVY | | | | NTATLT | | GV | | |
| | | RITC | | | | | | | | ISGVEA | | | | |
| | | | | | | | | | | GDEADY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81773 | 2937 | GACATCCGAATGACCCAGTCTCCTTCCTCCCTGTCTGCATCTG | 4629 | DIRMTQSPSSLSASV | 4983 | 11 | 10 |
| | | TAGGAGACAGAGTCACCATCACTTGCCGGGCAAGTCAGAGCAT | | GDRVTITCRASQSIG | | | |
| | | TGGCGGATATCTAAATTGGTATCAGCAGAAAACAGGGAGAGCC | | GYLNWYQQKTGRAPR | | | |
| | | CCTAGGCTACTGATCTTTGCTGCATCCAGACTGCAAAGTGGGG | | LLIFAASRLQSGVPS | | | |
| | | TCCCATCAAGGTTCAGTGGCAGTGGATCTGGGACAGAATTCAC | | RFSGSGSGTEFTLTI | | | |
| | | TCTCACCATCAGCGGTCTGCAACCTGACGATTTTGCAACTTAC | | SGLQPDDFATYYCQQ | | | |
| | | TACTGTCAACAGACTTACATTATGCCTCCGTGGACGTTCGGCC | | TYIMPPWTFGQGTKV | | | |
| | | AAGGGACCAAGGTGGAGATCAAA | | EIK | | | |
| ADI-81575 | 4592 | GACATCCAGATGACCCAGTCTCCATCCTCCCTGTCTGCATCTA | 4630 | DIQMTQSPSSLSASI | 4984 | 11 | 9 |
| | | TCGGAGACAGAGTCACCATCACTTGCCGGGCAAGTCAGAATAT | | GDRVTITCRASQNIS | | | |
| | | TAGTACCTATTTAAATTGGTATCAGCAAAAACCAGGGACAGCC | | TYLNWYQQKPGTAPN | | | |
| | | CCAAACCTCCTGATTTATGCTGCAGCCAGTTTGCACAGTGGTG | | LLIYAAASLHSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGGAACGTGAAGATTTTGCAACTTAC | | SSLEREDFATYYCQQ | | | |
| | | TACTGTCAGCAGAGTTACAGTACCCCGATCACCTTCGGCCTAG | | SYSTPITFGLGTRLE | | | |
| | | GGACACGACTGGAGATTAAA | | IK | | | |
| ADI-81727 | 2937 | GACATCGTGATGACCCAGTCTCCACTCTCTCTGTCCGTCACCC | 4631 | DIVMTQSPLSLSVTP | 4985 | 11 | 9 |
| | | CTGGTCAGTCGGCCTCCATCTCCTGCGAGTCCAGTAAGAGCCT | | GQSASISCESSKSLL | | | |
| | | CCTGCATAGTGGTGATGGAAAGACCTATTTGTATTGGTATTTG | | HSGDGKTYLYWYLQR | | | |
| | | CAGAGGCCAGGCCAGCCACCACAACTCCTGATCTATGAGGTTT | | PGQPPQLLIYEVSKR | | | |
| | | CCAAACGGTTCGCTGGAGTGCCAGATAGGTTCACTGGCAGCGG | | FAGVPDRFTGSGSGT | | | |
| | | GTCAGGGACGGATTTCACACTGAAAATCAGCCGGGTGGAGGCT | | DFTLKISRVEAEDVG | | | |
| | | GAGGATGTTGGGATTTATTACTGCATGCAGAGTATACAGCTTC | | IYYCMQSIQLPPFTF | | | |
| | | CTCCGTTCACTTTTGGCCAGGGGACCAAGGTGGAAATCAAA | | GQGTKVEIK | | | |
| ADI-81704 | 2940 | GAAATCACATTGACGCAGTCTCCAGGCACACTGTCTTTGTCTC | 4632 | EITLTQSPGTLSLSP | 4986 | 10 | 7 |
| | | CAGGGGAAAGAGCCACCCTCTCCTGCAGGGCCAGTGAGAGCGT | | GERATLSCRASESVS | | | |
| | | TAGCAGCGAGCACCTAGCCTGGCACCAGCAGAGACCTGGCCAG | | SEHLAWHQQRPGQAP | | | |
| | | GCTCCCAGGCTCCTCATCTATGGTGCATCCAAAAGGCCCACGG | | RLLIYGASKRPTGIP | | | |
| | | GCATCCCAGACAGGTTCAGTGGCAGTGGGTCTGGGACAGACTT | | DRFSGSGSGTDFTLT | | | |
| | | CACTCTCACCATCAGCAGACTGGAGCCTGAAGATTTTGCAGTG | | ISRLEPEDFAVYYCH | | | |
| | | TATTACTGTCACCAGTTTGCTAGCGCACCGATCACCTTCGGCC | | QFASAPITFGQGTRL | | | |
| | | AAGGGACACGACTGGAGATTAAA | | EIK | | | |
| ADI-81768 | 2947 | CAGCCTGTGATGACTCAGCCTCCCTCCGCGTCCGGGTCTCCTG | 4633 | QPVMTQPPSASGSPG | 4987 | 10 | 10 |
| | | GACAATCAGTCACCATCTCCTGCACTGGAACCAGCAGTGACGT | | QSVTISCTGTSSDVG | | | |
| | | TGGTGATTCTAACTATGTCTCCTGGTACCAACAACACCCAGGC | | DSNYVSWYQQHPGNP | | | |
| | | AACCCCCCCAAACTCATAATTTATGAGGTCAATAAGCGGCCCT | | PKLIIYEVNKRPSGV | | | |
| | | CAGGGGTCCCTGATCGTTTGTCTGGCTCCAAGTCTGGCAACAC | | PDRLSGSKSGNTASL | | | |
| | | GGCCTCCCTGACCGTCTCTGGGCTCCTGCCTGAGGATGAGGCT | | TVSGLLPEDEAHYYC | | | |
| | | CATTATTACTGCAGCTCAAATGCAGGCGTCACCGCCCTGATCT | | SSNAGVTALIFGGGT | | | |
| | | TCGGCGGAGGGACCAAGGTCACCGTCCTA | | KVTVL | | | |
| ADI-81775 | 2952 | GAAATTGTGATGACCCAGTCTCCATCCTCCCTGTCTGCATCTG | 4634 | EIVMTQSPSSLSASV | 4988 | 10 | 10 |
| | | TAGGAGACAGAGTCACCATCACTTGCCGGGCAAGTCAGACCAT | | GDRVTITCRASQTIN | | | |
| | | TAACAGCTATTTAAATTGGTATCAGCATAAACCAGGGAAAGCC | | SYLNWYQHKPGKAPK | | | |
| | | CCAAAACTCCTGATCTATGGTGCATCCACTTTACTAAGTGGGG | | LLIYGASTLLSGVPS | | | |
| | | TCCCATCAAGGTTCAGTGGGAGTGAATCTGGGACAGATTACAC | | RFSGSESGTDYTLTI | | | |
| | | TCTCACCATCAACGGTCTGCAACCTGAAGATTTTGCAACTTAC | | NGLQPEDFATYYCQQ | | | |
| | | TACTGTCAACAGACTTACAGTACACCTCCGTGGACGTTCGCCC | | TYSTPPWTFAQGTKV | | | |
| | | AAGGGACCAAGGTGGATATCAAA | | DIK | | | |
| ADI-81598 | 2937 | GACATCCAAATGACCCAGTCTCCATCCTCCCTGTCTGCATCTG | 4635 | DIQMTQSPSSLSASV | 4989 | 10 | 9 |
| | | TTGGGGACAGAGTCACCATCACTTGCCGGACAAGTCTGAACAT | | GDRVTITCRTSLNIK | | | |
| | | TAAGAACTATTTAAACTGGTATCAGCAGAAGCCAGGGAAAGCC | | NYLNWYQQKPGKAPK | | | |
| | | CCTAAGCTCCTGATCTATGCTGCATCCACTTTGCAAAGTGGGG | | LLIYAASTLQSGVPS | | | |
| | | TCCCATCAAGGTTCAGAGGTAGTGGTTCTGGGACAGATTTCAC | | RFRGSGSGTDFTLTI | | | |
| | | TCTCACCATCAGCGGTCTGCAACCAGAGGACTTTGCAACTTAC | | SGLQPEDFATYFCQQ | | | |
| | | TTCTGTCAACAGAGTTACGTTACCCCCCCGTGGACTTTTGGCC | | SYVTPPWTFGQGTKV | | | |
| | | AGGGGACCAAAGTGGAAATCAAA | | EIK | | | |
| ADI-81914 | 2958 | CAGTCTGTGCTGACGCAGCCGCCCTCGGTGTCACTGGCCCCAG | 4636 | QSVLTQPPSVSLAPG | 4990 | 10 | 8 |
| | | GACAGACGGCCAGGATTACCTGTGGGGGAGACAATATTGGAAG | | QTARITCGGDNIGSY | | | |
| | | TTATCGTGTGCACTGGTACCAGCGGAAGCCAGGCCAGGCCCCT | | RVHWYQRKPGQAPIL | | | |
| | | ATACTGGTCGTCTATGATGATAGCGACCGGCCCTCAGGGATCC | | VVYDDSDRPSGIPER | | | |
| | | CTGAGCGATTCTCTGGCTCCACCTCTGGGAACACGGCCACCCT | | FSGSTSGNTATLTIS | | | |
| | | GACCATCAGCGGGGTCGAGGCCGGGGATGAGGCCGACTATTAC | | GVEAGDEADYYCQVW | | | |
| | | TGTCAGGTGTGGGATACTAGTAGAAGTACTCATCATCGGGGAG | | DTSRSTHHRGVFGAG | | | |
| | | TCTTCGGAGCTGGGACCAAGCTCACCGTCCTA | | TKLTVL | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81637 | IGKV1D-39 | DIVLTQ | 3549 | RASQSI | 3738 | WYQQKP | 2257 | AASTLQ | 2392 | GVPSRF | 4180 | QQSYRS | 4353 | FGQGTK |
| | | SPSSLS | | NNYLN | | GKAPKL | | S | | AGSGSG | | RT | | VEIK |
| | | ASIGDR | | | | LIY | | | | TDFTLT | | | | |
| | | VTFTC | | | | | | | | ITSLQP | | | | |
| | | | | | | | | | | EDYATY | | | | |
| | | | | | | | | | | YC | | | | |
| ADI-81641 | IGKV3-20 | EIVLTQ | 3550 | RASQSV | 3739 | WFQQKP | 3926 | DASTRA | 2424 | GIPDRF | 4181 | QQFARS | 4354 | FGQGTK |
| | | SPGTLS | | TSS-LA | | GQAPRL | | T | | SGSGSG | | PRT | | VEIK |
| | | LSPGER | | | | LVY | | | | TDFTLT | | | | |
| | | ATLAC | | | | | | | | ISRLER | | | | |
| | | | | | | | | | | EDFAVY | | | | |
| | | | | | | | | | | YC | | | | |
| ADI-81729 | IGKV1-5 | DIPVTQ | 3551 | RASQSI | 3740 | WYQQKP | 3927 | KAFTLE | 4047 | GVPSKF | 4182 | QQYNRY | 4355 | FGPGTK |
| | | SPSTLS | | NNRLA | | GRAPKL | | S | | SGSGSG | | TWT | | VDIK |
| | | ASVGDR | | | | LIS | | | | TEFTLT | | | | |
| | | VTITC | | | | | | | | ISSLQP | | | | |
| | | | | | | | | | | DDFATY | | | | |
| | | | | | | | | | | YC | | | | |
| ADI-81643 | IGKV1D-39 | EIGMTQ | 3552 | RASQSI | 3741 | WYQQKP | 2263 | AASSLD | 4048 | GVPSRF | 4183 | QQSHRA | 4356 | FGQGTK |
| | | SPSSLS | | SSSYLN | | GKAPNL | | A | | SASGSG | | PAT | | VEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81701 | IGLV3-27 | SSVLIQ | 3553 | SGDLLA | 3742 | WFQQKP | 3928 | KDNERP | 4049 | GIPERF | 4184 | YSAAGN | 4357 | FGGGTK |
| | | PPSVSV | | NTYAR | | GQAPVL | | S | | SGSSSG | | NRE | | LTVL |
| | | SPGQTA | | | | FIY | | | | TTVTLT | | | | |
| | | SITC | | | | | | | | ISGAQV | | | | |
| | | | | | | | | | | DDEAVY | | | | |
| | | | | | | | | | | YC | | | | |
| ADI-81720 | IGLV1-40 | QSVLTQ | 3554 | TGSSSN | 3743 | WYQQLP | 3929 | DN- | 4050 | GVPDRF | 2599 | QSYDSS | 4358 | FGTGTK |
| | | PPSVSG | | IGAGHD | | GTAPRL | | NRPS | | SGSKSG | | LSIYYV | | LTVL |
| | | APGQTV | | VH | | LMF | | | | TSASLA | | | | |
| | | TISC | | | | | | | | ITGLQA | | | | |
| | | | | | | | | | | EDEADY | | | | |
| | | | | | | | | | | YC | | | | |
| ADI-81632 | IGKV1-NL1 | DIRLTQ | 3555 | RASQGI | 2062 | WYQQKP | 2296 | GASTLE | 4051 | GVPSRF | 4185 | QQYYST | 2739 | FGQGTK |
| | | SPSSLC | | SNSLA | | GKAPKF | | S | | SGSGCG | | PPRT | | VEIK |
| | | ASVGDR | | | | LLY | | | | TDFTLT | | | | |
| | | VSITC | | | | | | | | ISSLQP | | | | |
| | | | | | | | | | | EDFAIY | | | | |
| | | | | | | | | | | YC | | | | |
| ADI-81859 | IGKV1-33 | DIPMTQ | 1947 | QASQDI | 3744 | WYQQKP | 2257 | DASNLE | 2416 | GVPSRF | 4186 | QQYKSF | 4359 | FGGGTK |
| | | SPSSLS | | GTYLS | | GKAPKL | | T | | SGSGSG | | PPT | | VEIK |
| | | ASVGDR | | | | LIY | | | | TGSTLT | | | | |
| | | VTITC | | | | | | | | ISGLQP | | | | |
| | | | | | | | | | | EDFATY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81637 | 2937 | GATATTGTGCTGACCCAGTCTCCATCCTCCCTGTCTGCATCTA | 4637 | DIVLTQSPSSLSASI | 4991 | 10 | 8 |
| | | TTGGAGACAGAGTCACCTTCACTTGCCGGGCAAGTCAGAGCAT | | GDRVTFTCRASQSIN | | | |
| | | TAACAACTATTTAAATTGGTATCAGCAGAAACCAGGGAAAGCC | | NYLNWYQQKPGKAPK | | | |
| | | CCTAAGCTCCTGATTTATGCTGCATCCACTTTGCAGAGTGGGG | | LLIYAASTLQSGVPS | | | |
| | | TCCCATCAAGGTTCGCTGGCAGTGGATCCGGAACAGATTTCAC | | RFAGSGSGTDFTLTI | | | |
| | | TCTCACCATCACCAGTCTGCAACCTGAAGATTATGCAACTTAC | | TSLQPEDYATYYCQQ | | | |
| | | TACTGCCAACAGAGTTACAGGAGCCGGACGTTCGGCCAAGGGA | | SYRSRTFGQGTKVEI | | | |
| | | CCAAGGTGGAGATCAAA | | K | | | |
| ADI-81641 | 2937 | GAAATTGTGTTGACCCAGTCTCCAGGCACCCTGTCTTTGTCTC | 4638 | EIVLTQSPGTLSLSP | 4992 | 10 | 8 |
| | | CAGGGGAAAGAGCCACCCTCGCCTGCAGGGCCAGTCAGAGTGT | | GERATLACRASQSVT | | | |
| | | TACCAGCTCCTTAGCCTGGTTCCAGCAGAAACCTGGCCAGGCT | | SSLAWFQQKPGQAPR | | | |
| | | CCCAGGCTCCTCGTCTATGATGCATCCACCAGGGCCACTGGCA | | LLVYDASTRATGIPD | | | |
| | | TCCCAGACAGGTTCAGTGGCAGTGGGTCTGGGACTGACTTCAC | | RFSGSGSGTDFTLTI | | | |
| | | TCTCACCATCAGCAGACTGGAGCGTGAAGATTTTGCAGTGTAT | | SRLEREDFAVYYCQQ | | | |
| | | TACTGTCAGCAGTTTGCTAGGTCACCTCGCACTTTTGGCCAGG | | FARSPRTFGQGTKVE | | | |
| | | GGACCAAGGTGGAAATCAAA | | IK | | | |
| ADI-81729 | 2950 | GACATCCCGGTGACCCAGTCTCCTTCCACCCTGTCTGCATCTG | 4639 | DIPVTQSPSTLSASV | 4993 | 10 | 8 |
| | | TTGGAGACAGAGTCACCATCACTTGCCGGGCCAGTCAGAGTAT | | GDRVTITCRASQSIN | | | |
| | | TAATAATAGGTTGGCCTGGTATCAGCAGAAACCAGGGAGAGCC | | NRLAWYQQKPGRAPK | | | |
| | | CCTAAGCTCCTGATTTCTAAGGCGTTTACCTTAGAAAGTGGGG | | LLISKAFTLESGVPS | | | |
| | | TCCCATCAAAGTTCAGCGGCAGTGGATCTGGGACAGAATTCAC | | KFSGSGSGTEFTLTI | | | |
| | | CCTTACCATCAGCAGCCTGCAGCCTGATGATTTTGCAACTTAT | | SSLQPDDFATYYCQQ | | | |
| | | TATTGTCAACAGTATAATAGGTATACGTGGACGTTCGGCCCAG | | YNRYTWTFGPGTKVD | | | |
| | | GGACCAAAGTGGATATCAAA | | IK | | | |
| ADI-81643 | 2937 | GAAATCGGGATGACCCAGTCTCCATCCTCCCTGTCTGCATCTG | 4640 | EIGMTQSPSSLSASV | 4994 | 10 | 5 |
| | | TAGGAGACAGAGTCACCATCACTTGCCGGGCAAGTCAGAGCAT | | GDRVTITCRASQSIS | | | |
| | | TAGCAGCAGCTATTTAAATTGGTATCAGCAGAAACCAGGGAAA | | SSYLNWYQQKPGKAP | | | |
| | | GCCCCTAACCTCCTGATCTATGCTGCATCCAGTTTGGATGCAG | | NLLIYAASSLDAGVP | | | |
| | | GGGTCCCATCAAGGTTCAGTGCCAGTGGATCTGGGACAGATTT | | SRFSASGSGTDFTLT | | | |
| | | CACTCTCACCATCAGTAGTCTGCAACCTGAAGATTTTGCAACT | | ISSLQPEDFATYYCQ | | | |
| | | TACTACTGTCAACAGAGTCACAGGGCCCCCGCCACTTTTGGCC | | QSHRAPATFGQGTKV | | | |
| | | AGGGGACCAAGGTGGAGATCAAA | | EIK | | | |
| ADI-81701 | 2939 | TCGTCAGTGCTGATTCAGCCACCCTCAGTGTCAGTGTCTCCGG | 4641 | SSVLIQPPSVSVSPG | 4995 | 10 | 9 |
| | | GACAGACAGCCAGTATCACGTGCTCGGGAGACCTATTGGCAAA | | QTASITCSGDLLANT | | | |
| | | TACATATGCTCGGTGGTTCCAGCAGAAGCCAGGCCAGGCCCCT | | YARWFQQKPGQAPVL | | | |
| | | GTTTTGTTCATTTATAAAGACAATGAGCGGCCCTCAGGGATCC | | FIYKDNERPSGIPER | | | |
| | | CTGAGCGATTCTCCGGCTCCAGCTCAGGGACCACAGTCACCTT | | FSGSSSGTTVTLTIS | | | |
| | | GACCATCAGCGGGGCCCAGGTTGACGATGAGGCTGTCTATTAC | | GAQVDDEAVYYCYSA | | | |
| | | TGTTACTCTGCGGCCGGCAACAATCGAGAGTTCGGCGGAGGGA | | AGNNREFGGGTKLTV | | | |
| | | CCAAGCTCACCGTCCTA | | L | | | |
| ADI-81720 | 2949 | CAGTCTGTGTTGACGCAGCCGCCCTCAGTGTCTGGGGCCCCAG | 4642 | QSVLTQPPSVSGAPG | 4996 | 10 | 7 |
| | | GGCAGACGGTCACCATCTCCTGCACTGGGAGCAGCTCCAACAT | | QTVTISCTGSSSNIG | | | |
| | | CGGGGCAGGTCATGATGTACACTGGTATCAGCAGCTTCCAGGA | | AGHDVHWYQQLPGTA | | | |
| | | ACAGCCCCCAGACTCCTCATGTTTGACAACAATCGGCCCTCAG | | PRLLMFDNNRPSGVP | | | |
| | | GGGTCCCTGACCGATTCTCTGGCTCCAAGTCTGGCACCTCAGC | | DRFSGSKSGTSASLA | | | |
| | | CTCCCTGGCCATCACTGGGCTCCAGGCTGAGGATGAGGCTGAT | | ITGLQAEDEADYYCQ | | | |
| | | TATTACTGCCAGTCCTATGACAGCAGCCTGAGTATTTATTATG | | SYDSSLSIYYVFGTG | | | |
| | | TCTTCGGAACTGGGACCAAGCTCACCGTCCTA | | TKLTVL | | | |
| ADI-81632 | 2937 | GACATCCGGTTGACCCAGTCTCCATCCTCCCTGTGCGCATCTG | 4643 | DIRLTQSPSSLCASV | 4997 | 10 | 8 |
| | | TAGGAGACAGAGTCAGCATCACTTGCCGGGCGAGTCAGGGCAT | | GDRVSITCRASQGIS | | | |
| | | TAGCAATTCTTTAGCCTGGTATCAGCAAAAACCAGGGAAAGCC | | NSLAWYQQKPGKAPK | | | |
| | | CCTAAATTCCTGCTATATGGTGCATCCACTTTGGAAAGTGGGG | | FLLYGASTLESGVPS | | | |
| | | TCCCATCCAGGTTCAGTGGCAGTGGATGTGGGACGGATTTCAC | | RFSGSGCGTDFTLTI | | | |
| | | TCTCACCATCAGCAGCCTGCAGCCTGAAGATTTTGCAATTTAT | | SSLQPEDFAIYYCQQ | | | |
| | | TACTGTCAACAGTATTATAGTACTCCTCCGAGGACGTTCGGCC | | YYSTPPRTFGQGTKV | | | |
| | | AAGGGACCAAGGTGGAAATCAAA | | EIK | | | |
| ADI-81859 | 2936 | GACATCCCGATGACCCAGTCTCCATCCTCCCTGTCTGCATCTG | 4644 | DIPMTQSPSSLSASV | 4998 | 10 | 8 |
| | | TAGGAGACAGAGTCACCATCACTTGCCAGGCGAGTCAGGACAT | | GDRVTITCQASQDIG | | | |
| | | TGGCACCTATTTAAGTTGGTATCAACAGAAACCCGGAAAAGCC | | TYLSWYQQKPGKAPK | | | |
| | | CCTAAACTCCTGATCTACGATGCATCCAATTTAGAAACGGGGG | | LLIYDASNLETGVPS | | | |
| | | TCCCATCAAGGTTCAGTGGAAGTGGATCTGGGACAGGCTCGAC | | RFSGSGSGTGSTLTI | | | |
| | | TTTAACCATCAGCGGCCTGCAGCCTGAGGATTTTGCAACATAT | | SGLQPEDFATYYCQQ | | | |
| | | TACTGTCAACAATATAAAAGTTTCCCTCCGACTTTCGGCGGGG | | YKSFPPTFGGGTKVE | | | |
| | | GGACCAAGGTGGAAATCAAA | | IK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81891 | IGKV1-NL1 | EIELTQ | 3556 | RASHGI | 3745 | WYQQKP | 3930 | AASSLE | 4052 | GVPSRF | 4187 | QQYYSP | 4360 | FGQGTK |
| | | SPSSLS | | TDSLA | | GKAPRL | | T | | SGSGFG | | PNA | | VEIK |
| | | ASVGDR | | | | LLY | | | | TDFTLT | | | | |
| | | VTITQ | | | | | | | | ITSLQP | | | | |
| | | | | | | | | | | EDFATY | | | | |
| | | | | | | | | | | YC | | | | |
| ADI-81814 | IGKV1-NL1 | DIQVTQ | 2019 | RASHDI | 3746 | WYQQTL | 3931 | AASTLE | 2398 | GVPSRF | 2626 | QQYYGS | 4361 | FGQGTK |
| | | SPSSLS | | SKSLA | | GKAPKL | | S | | RGSGSG | | PRT | | VDIK |
| | | ASVGDR | | | | LLY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81602 | IGLV1-47 | SYELIQ | 3557 | SGSSSN | 3747 | WYQQLP | 3932 | RENLRP | 4053 | GVPDRI | 4188 | AAWDDS | 4362 | FGGGTK |
| | | PPSASG | | IGSNFV | | GTAPRL | | S | | SGSKSG | | LSGVV | | VTVL |
| | | TPGQRV | | Y | | LIY | | | | TSVSLA | | | | |
| | | TISC | | | | | | | | ISGLRA | | | | |
| | | | | | | | | | | EDEADY | | | | |
| | | | | | | | | | | YC | | | | |
| ADI-81572 | IGLV1-40 | QSVLTQ | 1948 | TGTTSN | 3748 | WYQQVP | 3933 | RNTNRP | 4054 | GVPDRF | 2622 | QSYDSS | 4363 | FGGGTK |
| | | PPSVSG | | IGAGYD | | GSAPKL | | S | | SGSKSD | | LTGVV | | LTVL |
| | | APGQRV | | AH | | IIY | | | | TSASLA | | | | |
| | | TISC | | | | | | | | ITGLQA | | | | |
| | | | | | | | | | | EDEADY | | | | |
| | | | | | | | | | | YC | | | | |
| ADI-81871 | IGKV1D-39 | DIPMTQ | 3558 | RAAQSI | 3749 | WYQQKP | 2257 | AASTLH | 4055 | GVPSRF | 2536 | QQSYVT | 4364 | FGPGTR |
| | | SPSSLS | | GTYLN | | GKAPKL | | S | | SGSGSG | | PSIT | | LEIK |
| | | ASVGDR | | | | LIY | | | | TDFTLT | | | | |
| | | VSFTC | | | | | | | | INSLQP | | | | |
| | | | | | | | | | | EDFATY | | | | |
| | | | | | | | | | | YC | | | | |
| ADI-81560 | IGKV3-20 | DIQLTQ | 3559 | RASQSV | 3750 | WYQQKP | 2258 | GPSSRA | 4056 | GIPARF | 4189 | QQYGDS | 4365 | FGQGTK |
| | | SPGSLS | | RSSYLA | | GQAPRL | | I | | SGSGSG | | PWT | | VEIK |
| | | LSPGER | | | | LIY | | | | TDFSLT | | | | |
| | | ATLFC | | | | | | | | ISRLEA | | | | |
| | | | | | | | | | | EDVAVY | | | | |
| | | | | | | | | | | YC | | | | |
| ADI-81802 | IGKV1D-39 | EIVVTQ | 3560 | RASQSI | 3751 | WYQQKP | 2371 | AASGLH | 4057 | GVPSRF | 4190 | QQSHSI | 4366 | FGQGTR |
| | | SPSSLS | | DNYLN | | GKAPEL | | I | | SGSGSG | | PIT | | LEIK |
| | | ASVGDR | | | | LIY | | | | TDFTLT | | | | |
| | | VTIIC | | | | | | | | ISGLQP | | | | |
| | | | | | | | | | | EDFATY | | | | |
| | | | | | | | | | | YC | | | | |
| ADI-81558 | IGLV3-27 | QSVVTQ | 3561 | SGDLVE | 3752 | WFQQKP | 3934 | RDRERP | 4058 | GIPERF | 4191 | YSAADN | 4367 | FGGGTK |
| | | PPSVSV | | KKYVR | | GQAPVL | | S | | SGSNSG | | NLGV | | VTVL |
| | | SPGQTA | | | | VIY | | | | TTVTLT | | | | |
| | | RITC | | | | | | | | ISGAQV | | | | |
| | | | | | | | | | | EDEADY | | | | |
| | | | | | | | | | | FC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81891 | 2937 | GAAATCGAGTTGACCCAGTCTCCATCCTCCCTGTCTGCATCTG | 4645 | EIELTQSPSSLSASV | 4999 | 10 | 9 |
| | | TAGGAGACAGAGTCACCATCACTTGCCGGGCGAGTCACGGCAT | | GDRVTITCRASHGIT | | | |
| | | TACTGATTCTTTAGCCTGGTATCAGCAGAAACCAGGGAAAGCC | | DSLAWYQQKPGKAPR | | | |
| | | CCTAGGCTCCTGCTCTATGCTGCATCCAGCTTGGAAACTGGGG | | LLLYAASSLETGVPS | | | |
| | | TCCCATCCCGGTTCAGTGGCAGTGGATTTGGGACGGATTTCAC | | RFSGSGFGTDFTLTI | | | |
| | | TCTCACCATCACCAGCCTGCAGCCTGAAGATTTTGCAACTTAT | | TSLQPEDFATYYCQQ | | | |
| | | TACTGTCAACAGTATTATTCTCCCCCCAACGCTTTTGGCCAGG | | YYSPPNAFGQGTKVE | | | |
| | | GGACCAAGGTGGAGATCAAA | | IK | | | |
| ADI-81814 | 2944 | GACATCCAGGTGACCCAGTCTCCATCCTCCCTGTCTGCATCTG | 4646 | DIQVTQSPSSLSASV | 5000 | 9 | 8 |
| | | TAGGAGACAGAGTCACCATCACTTGCCGGGCGAGTCACGACAT | | GDRVTITCRASHDIS | | | |
| | | TAGCAAATCTTTAGCCTGGTATCAGCAGACATTAGGGAAAGCC | | KSLAWYQQTLGKAPK | | | |
| | | CCTAAGCTCCTGCTCTATGCTGCCTCCACATTGGAAAGTGGGG | | LLLYAASTLESGVPS | | | |
| | | TCCCATCCAGGTTCCGTGGCAGTGGATCTGGGACGGATTTCAC | | RFRGSGSGTDFTLTI | | | |
| | | TCTCACCATCAGCAGCCTCCAGCCTGAAGATTTTGCAACTTAT | | SSLQPEDFATYYCQQ | | | |
| | | TACTGTCAACAGTATTATGGTTCCCCTCGAACGTTCGGCCAAG | | YYGSPRTFGQGTKVD | | | |
| | | GGACCAAAGTGGATATCAAA | | IK | | | |
| ADI-81602 | 2947 | TCCTATGAGCTGATACAGCCACCCTCAGCGTCTGGGACCCCCG | 4647 | SYELIQPPSASGTPG | 5001 | 9 | 7 |
| | | GGCAGAGGGTCACCATCTCTTGTTCTGGAAGCAGCTCCAACAT | | QRVTISCSGSSSNIG | | | |
| | | CGGGAGTAATTTTGTATATTGGTACCAGCAGCTCCCAGGAACG | | SNFVYWYQQLPGTAP | | | |
| | | GCCCCCAGACTCCTCATTTATAGGGAGAATCTTCGGCCCTCAG | | RLLIYRENLRPSGVP | | | |
| | | GGGTCCCTGACCGAATCTCTGGCTCCAAGTCTGGCACCTCAGT | | DRISGSKSGTSVSLA | | | |
| | | CTCCCTGGCCATCAGTGGGCTCCGGGCCGAGGATGAGGCTGAC | | ISGLRAEDEADYYCA | | | |
| | | TATTACTGTGCAGCATGGGATGACAGCCTGAGTGGAGTGGTCT | | AWDDSLSGVVFGGGT | | | |
| | | TCGGCGGAGGGACCAAGGTCACCGTCCTA | | KVTVL | | | |
| ADI-81572 | 2939 | CAGTCTGTGCTGACGCAGCCGCCCTCAGTGTCTGGGGCCCCAG | 4648 | QSVLTQPPSVSGAPG | 5002 | 9 | 9 |
| | | GGCAGAGGGTCACCATCTCCTGCACTGGGACCACCTCCAACAT | | QRVTISCTGTTSNIG | | | |
| | | CGGGGCAGGTTATGATGCACACTGGTACCAGCAGGTTCCAGGG | | AGYDAHWYQQVPGSA | | | |
| | | TCAGCCCCCAAACTCATCATCTATCGTAACACCAATCGGCCCT | | PKLIIYRNTNRPSGV | | | |
| | | CAGGAGTCCCTGACCGATTCTCTGGCTCCAAGTCTGACACCTC | | PDRFSGSKSDTSASL | | | |
| | | GGCCTCCCTGGCCATCACTGGGCTCCAGGCTGAGGATGAGGCT | | AITGLQAEDEADYYC | | | |
| | | GATTATTACTGCCAGTCCTATGACAGCAGCCTGACTGGTGTGG | | QSYDSSLTGVVFGGG | | | |
| | | TCTTCGGCGGAGGGACCAAGCTCACCGTCCTA | | TKLTVL | | | |
| ADI-81871 | 4593 | GACATCCCGATGACCCAGTCTCCATCCTCCCTGTCTGCATCTG | 4649 | DIPMTQSPSSLSASV | 5003 | 9 | 8 |
| | | TAGGAGACAGAGTCAGCTTCACTTGCCGGGCAGCTCAGAGCAT | | GDRVSFTCRAAQSIG | | | |
| | | CGGCACCTATTTAAATTGGTATCAACAGAAACCAGGGAAAGCC | | TYLNWYQQKPGKAPK | | | |
| | | CCTAAGCTCCTGATCTATGCTGCATCCACTTTGCACAGTGGCG | | LLIYAASTLHSGVPS | | | |
| | | TCCCATCAAGGTTCAGTGGCAGTGGATCTGGGACAGATTTCAC | | RFSGSGSGTDFTLTI | | | |
| | | TCTCACCATCAACAGTCTGCAACCTGAAGATTTTGCAACTTAC | | NSLQPEDFATYYCQQ | | | |
| | | TACTGTCAACAGAGTTACGTTACCCCCTCGATCACCTTCGGCC | | SYVTPSITFGPGTRL | | | |
| | | CAGGGACACGACTGGAGATTAAA | | EIK | | | |
| ADI-81560 | 2937 | GACATCCAGTTGACCCAGTCTCCAGGCAGTCTGTCTTTGTCTC | 4650 | DIQLTQSPGSLSLSP | 5004 | 9 | 8 |
| | | CAGGGGAAAGAGCCACCCTCTTCTGCAGGGCCAGCCAGAGTGT | | GERATLFCRASQSVR | | | |
| | | TCGCAGTAGTTATTTAGCCTGGTACCAGCAGAAACCTGGCCAG | | SSYLAWYQQKPGQAP | | | |
| | | GCTCCCAGGCTTCTCATCTATGGTCCATCCAGTAGGGCCACCG | | RLLIYGPSSRATGIP | | | |
| | | GCATCCCAGCCAGGTTCAGTGGCAGTGGATCTGGGACAGACTT | | ARFSGSGSGTDFSLT | | | |
| | | CAGTCTCACCATCAGCAGACTGGAGGCTGAAGATGTTGCAGTG | | ISRLEAEDVAVYYCQ | | | |
| | | TATTATTGTCAGCAGTATGGTGACTCACCGTGGACGTTCGGCC | | QYGDSPWTFGQGTKV | | | |
| | | AAGGGACCAAGGTGGAAATCAAA | | EIK | | | |
| ADI-81802 | 2940 | GAAATTGTAGTGACCCAGTCTCCATCCTCCCTGTCTGCCTCTG | 4651 | EIVVTQSPSSLSASV | 5005 | 9 | 8 |
| | | TAGGAGACAGAGTCACCATCATTTGCCGGGCAAGTCAGAGCAT | | GDRVTIICRASQSID | | | |
| | | TGACAACTATTTAAATTGGTATCAGCAGAAACCAGGGAAAGCC | | NYLNWYQQKPGKAPE | | | |
| | | CCTGAGCTCCTGATCTATGCTGCATCCGGTTTACACATTGGGG | | LLIYAASGLHIGVPS | | | |
| | | TCCCATCAAGGTTCAGTGGCAGTGGATCTGGGACAGATTTCAC | | RFSGSGSGTDFTLTI | | | |
| | | TCTCACCATCAGCGGTCTGCAGCCTGAAGATTTTGCAACTTAT | | SGLQPEDFATYYCQQ | | | |
| | | TACTGTCAACAGAGTCACAGTATCCCGATCACCTTCGGCCAAG | | SHSIPITFGQGTRLE | | | |
| | | GGACACGACTGGAGATTAAA | | IK | | | |
| ADI-81558 | 2947 | CAGTCTGTGGTGACGCAGCCGCCCTCAGTGTCAGTGTCTCCGG | 4652 | QSVVTQPPSVSVSPG | 5006 | 9 | 9 |
| | | GACAGACAGCCAGGATCACCTGCTCAGGAGATTTAGTGGAAAA | | QTARITCSGDLVEKK | | | |
| | | AAAATATGTTCGGTGGTTCCAGCAGAAGCCAGGCCAGGCCCCT | | YVRWFQQKPGQAPVL | | | |
| | | GTACTGGTGATTTATAGAGACCGTGAGCGGCCCTCAGGGATCC | | VIYRDRERPSGIPER | | | |
| | | CTGAGCGATTCTCCGGCTCCAACTCAGGGACCACGGTCACCTT | | FSGSNSGTTVTLTIS | | | |
| | | GACCATCAGCGGGGCCCAGGTTGAGGATGAGGCTGACTATTTC | | GAQVEDEADYFCYSA | | | |
| | | TGTTACTCTGCGGCTGACAACAATTTGGGGGTGTTCGGCGGAG | | ADNNLGVFGGGTKVT | | | |
| | | GGACCAAGGTCACCGTCCTA | | VL | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81695 | IGLV3-21 | YYELTQ | 3562 | GGKNIG | 3753 | WYQQKP | 2283 | DDSDRP | 2399 | GIPERL | 4192 | QVWDSS | 4368 | FGGGTK |
| | | PPSVSV | | GQSVQ | | GQAPVL | | S | | SGSKSG | | SDHPG | | VTVL |
| | | APGQTA | | | | VVY | | | | TTATLT | | | | |
| | | RITQ | | | | | | | | ITRVEA | | | | |
| | | | | | | | | | | GDEADY | | | | |
| | | | | | | | | | | SC | | | | |
| ADI-81719 | IGKV3-11 | EIALTQ | 1930 | RASQNV | 3754 | WYQHKP | 2328 | DASRRA | 4059 | GIAARF | 4193 | QQRSNW | 4369 | FGGGTK |
| | | SPATLS | | SNYLG | | GQAPRL | | T | | SGSGSG | | PLT | | VEIK |
| | | LSPGER | | | | LIY | | | | TDFTLT | | | | |
| | | ATLSC | | | | | | | | ISSLEP | | | | |
| | | | | | | | | | | EDFVVY | | | | |
| | | | | | | | | | | YC | | | | |
| ADI-81758 | IGKV1-9 | EMTLTQ | 3563 | RASQGI | 2140 | WYQQRP | 2278 | GASTLQ | 2447 | GVPLRF | 4194 | QHLDSY | 2792 | FGQGTK |
| | | SPSFLS | | SSFLA | | GKAPKL | | S | | SGSGSV | | PRYT | | VEIK |
| | | ASVGDR | | | | LIF | | | | TEFTLT | | | | |
| | | VAITC | | | | | | | | ISGLQP | | | | |
| | | | | | | | | | | EDSATY | | | | |
| | | | | | | | | | | YC | | | | |
| ADI-81780 | IGKV1-NL1 | DIGMTQ | 3564 | RASQGI | 2067 | WYQQRP | 3935 | AASTLE | 2398 | GVPPRF | 4195 | QQYYSP | 2779 | FGQGTK |
| | | SPSSLS | | TNSLA | | GKAPTL | | S | | SGGGSG | | PPRT | | VEIK |
| | | ASVGDR | | | | LLY | | | | TVFTLT | | | | |
| | | VTITC | | | | | | | | ISSLQP | | | | |
| | | | | | | | | | | EDFATY | | | | |
| | | | | | | | | | | YC | | | | |
| ADI-81574 | IGKV1D-39 | DIQMTQ | 1899 | RASQMI | 3755 | WYQQKP | 2257 | GASSLQ | 2403 | GVPSRF | 4196 | QQSYST | 2741 | FGQGTK |
| | | SPSSLP | | RTFLN | | GKAPKL | | S | | SGTGSG | | PPWT | | VEIK |
| | | ASVGDR | | | | LIY | | | | TDFTLT | | | | |
| | | VTITC | | | | | | | | ISSLQP | | | | |
| | | | | | | | | | | EDFAIY | | | | |
| | | | | | | | | | | YC | | | | |
| ADI-81698 | IGLV2-14 | QSVLTQ | 1918 | TGTSSD | 3756 | WYQQYP | 3936 | EVTYRP | 4060 | GISGRF | 4197 | SSYRSS | 4370 | FGGGTK |
| | | PASVSG | | IGAYNY | | GKAPKL | | S | | SGSKSG | | ITSPS | | LTVL |
| | | SPGQSI | | VS | | IIY | | | | NTASLT | | | | |
| | | TISC | | | | | | | | ISGLQA | | | | |
| | | | | | | | | | | EDEADY | | | | |
| | | | | | | | | | | YC | | | | |
| ADI-81831 | IGKV3-20 | ETTLTQ | 3565 | RASQSI | 3757 | WYQQKP | 2258 | GASTRA | 2383 | GIPDRF | 4198 | QHYGSS | 4371 | FGQGTK |
| | | SPGTLS | | SDSRYL | | GQAPRL | | T | | SGSGSG | | LWT | | VEIK |
| | | VFPGER | | A | | LIY | | | | TDFTLT | | | | |
| | | ATLSC | | | | | | | | VSRLEP | | | | |
| | | | | | | | | | | EDFAVY | | | | |
| | | | | | | | | | | YC | | | | |
| ADI-81712 | IGLV1-44 | QSVLTQ | 3566 | SGHFSN | 3758 | WYQQVP | 3937 | NNNQRP | 4061 | GVPDRF | 2615 | AVWDDS | 4372 | FGGGTK |
| | | PPSASG | | IGDNTV | | GTAPKL | | S | | SGSKSG | | LDGPV | | VTVL |
| | | TPGQRV | | N | | LIH | | | | TSASLA | | | | |
| | | TISC | | | | | | | | ISGLQS | | | | |
| | | | | | | | | | | EDEADY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81695 | 2947 | TATTATGAGCTGACTCAGCCACCTTCGGTGTCAGTGGCCCCAG | 4653 | YYELTQPPSVSVAPG | 5007 | 9 | 9 |
| | | GACAGACGGCCAGGATTACCTGTGGGGGAAAAAACATTGGAGG | | QTARITCGGKNIGGQ | | | |
| | | TCAAAGTGTGCAGTGGTACCAGCAGAAGCCAGGCCAGGCCCCT | | SVQWYQQKPGQAPVL | | | |
| | | GTGCTGGTCGTCTATGATGATAGCGACCGGCCCTCAGGGATCC | | VVYDDSDRPSGIPER | | | |
| | | CTGAGCGATTATCTGGTTCCAAGTCTGGGACCACGGCCACCCT | | LSGSKSGTTATLTIT | | | |
| | | GACCATCACCAGGGTCGAAGCCGGGGATGAGGCCGACTATTCC | | RVEAGDEADYSCQVW | | | |
| | | TGTCAGGTGTGGGATAGTAGTAGTGATCATCCAGGCTTCGGCG | | DSSSDHPGFGGGTKV | | | |
| | | GAGGGACCAAGGTGACCGTCCTA | | TVL | | | |
| ADI-81719 | 2936 | GAAATCGCATTGACGCAGTCTCCAGCCACCCTGTCTTTGTCTC | 4654 | EIALTQSPATLSLSP | 5008 | 9 | 7 |
| | | CGGGGGAAAGAGCCACCCTCTCCTGCAGGGCCAGTCAGAATGT | | GERATLSCRASQNVS | | | |
| | | TAGCAACTACTTAGGTTGGTACCAACATAAACCTGGCCAGGCT | | NYLGWYQHKPGQAPR | | | |
| | | CCCAGGCTCCTCATTTATGATGCATCCAGGAGGGCCACTGGCA | | LLIYDASRRATGIAA | | | |
| | | TCGCAGCCAGGTTCAGTGGCAGTGGGTCTGGGACAGACTTCAC | | RFSGSGSGTDFTLTI | | | |
| | | TCTCACCATCAGCAGCCTAGAGCCTGAAGATTTTGTAGTTTAT | | SSLEPEDFVVYYCQQ | | | |
| | | TACTGTCAGCAGCGTAGCAACTGGCCGCTCACTTTCGGCGGAG | | RSNWPLTFGGGTKVE | | | |
| | | GGACCAAGGTGGAAATCAAA | | IK | | | |
| ADI-81758 | 2937 | GAAATGACACTCACGCAGTCTCCATCCTTCCTGTCTGCATCTG | 4655 | EMTLTQSPSFLSASV | 5009 | 9 | 9 |
| | | TAGGAGACAGAGTCGCCATCACTTGCCGGGCCAGTCAGGGCAT | | GDRVAITCRASQGIS | | | |
| | | TAGCAGTTTTTTAGCCTGGTATCAGCAAAGACCAGGGAAAGCC | | SFLAWYQQRPGKAPK | | | |
| | | CCTAAGCTCCTGATCTTTGGTGCATCCACTTTGCAAAGTGGGG | | LLIFGASTLQSGVPL | | | |
| | | TCCCATTAAGGTTCAGCGGCAGTGGATCTGTGACAGAATTCAC | | RFSGSGSVTEFTLTI | | | |
| | | TCTCACAATCAGCGGCCTGCAGCCTGAAGATTCTGCAACTTAT | | SGLQPEDSATYYCQH | | | |
| | | TACTGTCAACACCTTGATAGTTACCCTCGGTACACTTTTGGCC | | LDSYPRYTFGQGTKV | | | |
| | | AGGGGACCAAGGTGGAGATCAAA | | EIK | | | |
| ADI-81780 | 2937 | GACATCGGGATGACCCAGTCTCCGTCCTCCCTGTCTGCATCTG | 4656 | DIGMTQSPSSLSASV | 5010 | 9 | 8 |
| | | TAGGAGACAGAGTCACCATCACTTGCCGGGCGAGTCAGGGCAT | | GDRVTITCRASQGIT | | | |
| | | TACCAATTCTTTAGCCTGGTATCAGCAAAGACCAGGGAAAGCC | | NSLAWYQQRPGKAPT | | | |
| | | CCAACCCTCCTTCTCTATGCTGCATCCACACTGGAAAGTGGGG | | LLLYAASTLESGVPP | | | |
| | | TCCCACCCAGGTTCAGTGGCGGTGGATCTGGGACGGTTTTCAC | | RFSGGGSGTVFTLTI | | | |
| | | TCTCACCATCAGCAGCCTGCAGCCTGAAGATTTTGCAACTTAT | | SSLQPEDFATYYCQQ | | | |
| | | TACTGTCAGCAGTACTATAGTCCCCCTCCCCGGACGTTCGGCC | | YYSPPPRTFGQGTKV | | | |
| | | AAGGGACCAAGGTGGAAATCAAA | | EIK | | | |
| ADI-81574 | 2937 | GACATCCAGATGACCCAGTCTCCATCCTCCCTGCCTGCATCTG | 4657 | DIQMTQSPSSLPASV | 5011 | 9 | 8 |
| | | TGGGAGACAGAGTCACCATCACTTGCCGGGCAAGTCAGATGAT | | GDRVTITCRASQMIR | | | |
| | | TAGGACCTTTTTAAATTGGTATCAGCAGAAGCCAGGGAAAGCC | | TFLNWYQQKPGKAPK | | | |
| | | CCTAAGCTCCTGATCTATGGTGCATCCAGTTTGCAAAGTGGGG | | LLIYGASSLQSGVPS | | | |
| | | TCCCATCAAGGTTCAGTGGCACTGGATCTGGGACAGATTTCAC | | RFSGTGSGTDFTLTI | | | |
| | | TCTCACCATCAGCAGTCTGCAACCTGAAGATTTTGCAATTTAC | | SSLQPEDFAIYYCQQ | | | |
| | | TACTGTCAACAGAGTTACAGTACCCCTCCGTGGACGTTCGGCC | | SYSTPPWTFGQGTKV | | | |
| | | AAGGGACCAAGGTGGAAATCAAA | | EIK | | | |
| ADI-81698 | 2939 | CAGTCTGTTCTGACTCAGCCTGCCTCCGTGTCTGGGTCTCCTG | 4658 | QSVLTQPASVSGSPG | 5012 | 9 | 8 |
| | | GACAGTCGATCACCATCTCCTGCACTGGGACCAGCAGTGACAT | | QSITISCTGTSSDIG | | | |
| | | TGGTGCTTATAACTATGTCTCCTGGTACCAACAGTACCCAGGC | | AYNYVSWYQQYPGKA | | | |
| | | AAAGCCCCCAAACTCATAATTTATGAGGTCACTTATCGGCCCT | | PKLIIYEVTYRPSGI | | | |
| | | CAGGGATTTCTGGTCGCTTCTCTGGCTCCAAGTCTGGTAACAC | | SGRFSGSKSGNTASL | | | |
| | | GGCCTCCCTGACCATCTCTGGGCTCCAGGCTGAGGACGAGGCT | | TISGLQAEDEADYYC | | | |
| | | GATTATTACTGCAGCTCATATAGAAGTAGCACTTCTCCCTCAT | | SSYRSSTSPSFGGGT | | | |
| | | TCGGCGGTGGGACCAAGCTCACCGTCCTA | | KLTVL | | | |
| ADI-81831 | 2937 | GAAACGACACTCACGCAGTCTCCAGGCACGCTGTCTGTGTTTC | 4659 | ETTLTQSPGTLSVFP | 5013 | 9 | 7 |
| | | CAGGGGAAAGAGCCACCCTCTCCTGCAGGGCCAGTCAGAGTAT | | GERATLSCRASQSIS | | | |
| | | TAGCGACAGCAGATATTTAGCCTGGTACCAGCAGAAACCTGGC | | DSRYLAWYQQKPGQA | | | |
| | | CAGGCTCCCAGACTCCTCATCTATGGTGCATCCACCAGGGCCA | | PRLLIYGASTRATGI | | | |
| | | CTGGCATCCCAGACAGGTTCAGTGGCAGTGGGTCTGGGACAGA | | PDRFSGSGSGTDFTL | | | |
| | | CTTCACTCTCACCGTCAGCAGACTGGAGCCTGAAGATTTTGCA | | TVSRLEPEDFAVYYC | | | |
| | | GTTTATTACTGTCAGCACTATGGTAGCTCACTGTGGACGTTCG | | QHYGSSLWTFGQGTK | | | |
| | | GCCAAGGGACCAAGGTGGAAATCAAA | | VEIK | | | |
| ADI-81712 | 2947 | CAGTCTGTGCTGACGCAGCCGCCCTCAGCGTCTGGGACCCCCG | 4660 | QSVLTQPPSASGTPG | 5014 | 9 | 6 |
| | | GGCAGAGGGTCACCATCTCCTGTTCTGGGCACTTCTCCAACAT | | QRVTISCSGHFSNIG | | | |
| | | CGGAGATAATACTGTTAACTGGTACCAGCAGGTCCCAGGAACG | | DNTVNWYQQVPGTAP | | | |
| | | GCCCCCAAACTCCTCATACATAATAATAATCAGCGGCCCTCAG | | KLLIHNNNQRPSGVP | | | |
| | | GGGTCCCTGACCGCTTCTCTGGCTCCAAGTCTGGCACCTCAGC | | DRFSGSKSGTSASLA | | | |
| | | CTCCCTGGCCATCAGTGGGCTCCAGTCTGAGGATGAGGCTGAT | | ISGLQSEDEADYYCA | | | |
| | | TATTACTGTGCAGTATGGGATGACAGCCTGGATGGTCCGGTGT | | VWDDSLDGPVFGGGT | | | |
| | | TCGGCGGAGGGACCAAGGTGACCGTCCTC | | KVTVL | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81746 | IGKV1D-39 | EITLTQ | 3567 | RASQNI | 3759 | WYQQKP | 2371 | AASTLQ | 2417 | GVPSRF | 4199 | QQSHST | 4373 | FGPGTK |
| | | SPSSLS | | VGYLN | | GKAPEL | | R | | SGSGSE | | PFT | | VEIK |
| | | ASVGDR | | | | LIY | | | | TDFTLT | | | | |
| | | VTITC | | | | | | | | ISSLQP | | | | |
| | | | | | | | | | | EDIATY | | | | |
| | | | | | | | | | | YC | | | | |
| ADI-81752 | IGKV1-12 | DIEMTQ | 3568 | RASQDI | 3760 | WYQQKS | 3938 | DASSLE | 2434 | GVPSRF | 4200 | LQANSF | 4374 | FGPGTK |
| | | SPSSVS | | RSRLG | | GKAPKL | | S | | SGSGYG | | SLA | | VEIK |
| | | ASVGDR | | | | LIF | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81836 | IGKV1D-39 | EIQLTQ | 3569 | RAGHSI | 3761 | WYQQKP | 3939 | VSSSLQ | 4062 | GVPSRF | 4201 | QQSFSP | 4375 | FGQGTK |
| | | SPSSLS | | RNYLN | | GTAPKL | | S | | SGSGSG | | PYT | | VDIK |
| | | ASLGDR | | | | LIY | | | | TDFTLT | | | | |
| | | VTITC | | | | | | | | ISSLQP | | | | |
| | | | | | | | | | | EDSATY | | | | |
| | | | | | | | | | | YC | | | | |
| ADI-81864 | IGKV1-17 | DIQVTQ | 3570 | RASQGI | 2187 | WFQQKP | 3940 | GASSLQ | 4063 | GVALRF | 4202 | LQHNSS | 4376 | FGQGTR |
| | | SPSSMS | | SNYLA | | GKVPKR | | A | | SGSGSG | | PIT | | VEIK |
| | | ASVGDR | | | | LIY | | | | TEFTFT | | | | |
| | | VTITC | | | | | | | | ITSLQP | | | | |
| | | | | | | | | | | EDFATY | | | | |
| | | | | | | | | | | YC | | | | |
| ADI-81722 | IGKV1-33 | EIVMTQ | 3571 | QASQDI | 3762 | WYQHKP | 3924 | AASNLE | 4064 | GVPSRF | 2528 | QQFDIL | 4377 | FGQGTK |
| | | SPSSLS | | RSFLN | | GKAPKL | | I | | SGSGSG | | PWT | | VDIK |
| | | ASVGDR | | | | LIY | | | | TDFTFT | | | | |
| | | VAITC | | | | | | | | ISSLQP | | | | |
| | | | | | | | | | | EDFATY | | | | |
| | | | | | | | | | | YC | | | | |
| ADI-81595 | IGLV1-40 | SYELTQ | 3572 | TGSDSN | 3763 | WYRQLP | 3941 | GDNNRP | 2524 | GVPDRF | 4203 | | 2908 | FGGGTK |
| | | PPSVSG | | IGAGYD | | GTAPKL | | S | | SGSKSG | | QSYDSS | | LTVL |
| | | APGLRV | | VH | | LIY | | | | ISASLA | | LSGSV | | |
| | | TISC | | | | | | | | ITGLQA | | | | |
| | | | | | | | | | | EDEADY | | | | |
| | | | | | | | | | | YC | | | | |
| ADI-81587 | IGKV1-5 | EMTLTQ | 3573 | RASQNI | 3764 | WYQQKP | 3942 | DASSLE | 2388 | GVPSRF | 2532 | QQYKTY | 4378 | FGQGTK |
| | | SPPSLA | | NNWLA | | GKAPKL | | G | | SGSGSG | | ST | | VDIK |
| | | ASVGDR | | | | LIF | | | | TEFTLT | | | | |
| | | VTITC | | | | | | | | ISSLQP | | | | |
| | | | | | | | | | | DDFATY | | | | |
| | | | | | | | | | | YC | | | | |
| ADI-81597 | IGLV3-21 | YYVLTQ | 3574 | GGSDIG | 3765 | WYQQRP | 3943 | DDTDRP | 2419 | GIPERF | 4204 | QVWDVG | 4379 | FGGGTK |
| | | PPSVSV | | SKGVH | | GQAPVL | | S | | LGSNSG | | AV | | VTVL |
| | | APGQTA | | | | VVN | | | | NTATLT | | | | |
| | | RITC | | | | | | | | ISRVEA | | | | |
| | | | | | | | | | | GDEADY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81746 | 2942 | GAAATCACACTGACCCAGTCTCCATCCTCCCTGTCAGCGTCTG | 4661 | EITLTQSPSSLSASV | 5015 | 9 | 8 |
| | | TAGGAGACAGAGTCACCATCACTTGCCGGGCAAGTCAGAACAT | | GDRVTITCRASQNIV | | | |
| | | TGTCGGCTATTTAAATTGGTATCAGCAGAAACCAGGGAAAGCC | | GYLNWYQQKPGKAPE | | | |
| | | CCTGAGCTCCTGATCTATGCTGCGTCCACTTTGCAAAGAGGGG | | LLIYAASTLQRGVPS | | | |
| | | TCCCATCAAGGTTCAGTGGCAGTGGATCTGAGACAGATTTCAC | | RFSGSGSETDFTLTI | | | |
| | | TCTCACCATCAGCAGTCTGCAACCTGAAGATATTGCAACCTAC | | SSLQPEDIATYYCQQ | | | |
| | | TACTGTCAACAGAGTCACAGTACCCCCTTCACTTTCGGCCCTG | | SHSTPFTFGPGTKVE | | | |
| | | GGACCAAGGTGGAAATCAAA | | IK | | | |
| ADI-81752 | 2942 | GACATCGAGATGACCCAGTCTCCATCTTCCGTGTCTGCATCTG | 4662 | DIEMTQSPSSVSASV | 5016 | 9 | 9 |
| | | TAGGAGACAGAGTCACCATCACTTGTCGGGCGAGTCAGGATAT | | GDRVTITCRASQDIR | | | |
| | | TCGCAGCCGGTTAGGCTGGTATCAGCAGAAATCAGGGAAAGCC | | SRLGWYQQKSGKAPK | | | |
| | | CCTAAACTCCTGATCTTTGATGCATCCAGTTTGGAAAGTGGGG | | LLIFDASSLESGVPS | | | |
| | | TCCCATCAAGGTTCAGCGGCAGTGGCTATGGGACAGACTTCAC | | RFSGSGYGTDFTLTI | | | |
| | | TCTTACCATCAGCAGCCTGCAGCCTGAAGATTTTGCAACTTAC | | SSLQPEDFATYYCLQ | | | |
| | | TATTGTCTACAGGCTAATAGTTTCTCTCTCGCTTTCGGCCCTG | | ANSFSLAFGPGTKVE | | | |
| | | GACCAAGGTGGAGATCAAA | | IK | | | |
| ADI-81836 | 2944 | GAAATCCAGTTGACCCAGTCTCCATCCTCCCTGTCAGCATCTT | 4663 | EIQLTQSPSSLSASL | 5017 | 9 | 9 |
| | | TAGGAGACAGAGTCACCATCACTTGCCGGGCAGGTCACAGCAT | | GDRVTITCRAGHSIR | | | |
| | | TAGGAACTATTTAAATTGGTATCAGCAGAAACCAGGGACAGCC | | NYLNWYQQKPGTAPK | | | |
| | | CCTAAGCTCCTGATCTATGTTTCATCCAGTTTGCAAAGTGGGG | | LLIYVSSSLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAACCTGAGGATTCTGCAACTTAC | | SSLQPEDSATYYCQQ | | | |
| | | TACTGTCAACAGAGTTTCAGTCCCCCTTACACTTTTGGCCAGG | | SFSPPYTFGQGTKVD | | | |
| | | GGACCAAGGTGGATATCAAA | | IK | | | |
| ADI-81864 | 4594 | GACATCCAGGTGACCCAGTCTCCATCCTCCATGTCTGCATCTG | 4664 | DIQVTQSPSSMSASV | 5018 | 9 | 7 |
| | | TAGGAGACAGAGTCACCATCACTTGTCGGGCGAGTCAGGGCAT | | GDRVTITCRASQGIS | | | |
| | | TAGCAATTATTTAGCCTGGTTTCAGCAGAAACCAGGGAAAGTC | | NYLAWFQQKPGKVPK | | | |
| | | CCTAAGCGCCTGATCTATGGTGCATCCAGTTTGCAAGCAGGGG | | RLIYGASSLQAGVAL | | | |
| | | TCGCATTAAGGTTCAGCGGCAGTGGATCTGGGACAGAATTCAC | | RFSGSGSGTEFTFTI | | | |
| | | TTTCACCATCACCAGCCTGCAGCCTGAAGATTTTGCAACATAT | | TSLQPEDFATYYCLQ | | | |
| | | TACTGTCTACAACATAATAGTTCTCCTATCACCTTCGGCCAAG | | HNSSPITFGQGTRVE | | | |
| | | GGACCCGAGTGGAGATTAAA | | IK | | | |
| ADI-81722 | 2944 | GAAATCGTGATGACGCAGTCTCCATCCTCCCTGTCTGCATCTG | 4665 | EIVMTQSPSSLSASV | 5019 | 8 | 8 |
| | | TAGGAGACAGAGTCGCCATCACTTGCCAGGCGAGTCAGGACAT | | GDRVAITCQASQDIR | | | |
| | | TAGGAGCTTTTTAAATTGGTATCAGCATAAACCAGGGAAAGCC | | SFLNWYQHKPGKAPK | | | |
| | | CCTAAGCTCCTGATCTACGCTGCATCCAATTTGGAAATAGGGG | | LLIYAASNLEIGVPS | | | |
| | | TCCCATCAAGGTTCAGTGGAAGTGGATCTGGGACAGATTTTAC | | RFSGSGSGTDFTFTI | | | |
| | | TTTCACCATCAGCAGCCTGCAGCCTGAAGATTTTGCAACATAT | | SSLQPEDFATYYCQQ | | | |
| | | TACTGTCAACAGTTTGATATTCTCCCCTGGACTTTTGGCCAGG | | FDILPWTFGQGTKVD | | | |
| | | GGACCAAGGTGGATATCAAA | | IK | | | |
| ADI-81595 | 2939 | TCGTATGAGCTGACTCAGCCACCCTCAGTGTCTGGGGCCCCAG | 4666 | SYELTQPPSVSGAPG | 5020 | 8 | 6 |
| | | GGCTGAGGGTCACCATCTCCTGCACTGGGAGCGACTCCAACAT | | LRVTISCTGSDSNIG | | | |
| | | CGGGGCAGGATATGATGTACACTGGTACCGGCAGCTTCCAGGG | | AGYDVHWYRQLPGTA | | | |
| | | ACAGCCCCCAAACTCCTCATCTATGGTGACAATAATCGGCCCT | | PKLLIYGDNNRPSGV | | | |
| | | CAGGGGTCCCTGACCGATTCTCTGGCTCCAAGTCTGGCATCTC | | PDRFSGSKSGISASL | | | |
| | | AGCCTCCCTGGCCATCACTGGTCTCCAGGCTGAGGATGAGGCT | | AITGLQAEDEADYYC | | | |
| | | GATTATTACTGCCAGTCCTATGACAGCAGCCTGAGTGGCAGCG | | QSYDSSLSGSVFGGG | | | |
| | | TTTTCGGCGGGGGGACCAAGCTCACCGTCCTA | | TKLTVL | | | |
| ADI-81587 | 2944 | GAAATGACACTCACGCAGTCTCCCCCCTCCCTGGCTGCATCTG | 4667 | EMTLTQSPPSLAASV | 5021 | 8 | 8 |
| | | TAGGAGACAGAGTCACCATCACTTGCCGGGCCAGTCAGAATAT | | GDRVTITCRASQNIN | | | |
| | | TAATAACTGGTTGGCCTGGTATCAGCAGAAACCAGGAAAAGCC | | NWLAWYQQKPGKAPK | | | |
| | | CCTAAGCTCCTGATCTTTGATGCCTCCAGTTTGGAAGGTGGGG | | LLIFDASSLEGGVPS | | | |
| | | TCCCATCAAGGTTCAGCGGCAGTGGATCTGGGACAGAATTCAC | | RFSGSGSGTEFTLTI | | | |
| | | TCTCACCATCAGCAGCCTGCAGCCTGATGATTTTGCAACTTAC | | SSLQPDDFATYYCQQ | | | |
| | | TACTGCCAGCAGTATAAAACTTACTCGACGTTCGGCCAAGGGA | | YKTYSTFGQGTKVDI | | | |
| | | CCAAGGTGGATATCAAA | | K | | | |
| ADI-81597 | 2947 | TACTATGTGCTGACTCAGCCACCCTCGGTGTCAGTGGCCCCAG | 4668 | YYVLTQPPSVSVAPG | 5022 | 8 | 7 |
| | | GACAGACGGCCAGAATTACCTGTGGGGGAAGCGACATTGGAAG | | QTARITCGGSDIGSK | | | |
| | | TAAGGGTGTGCACTGGTACCAACAGAGGCCAGGCCAGGCCCCT | | GVHWYQQRPGQAPVL | | | |
| | | GTGCTGGTCGTCAATGATGATACCGACCGGCCCTCAGGGATTC | | VVNDDTDRPSGIPER | | | |
| | | CTGAGCGATTCCTTGGCTCCAACTCTGGGAACACGGCCACCCT | | FLGSNSGNTATLTIS | | | |
| | | GACCATCAGCAGGGTCGAGGCCGGAGATGAGGCCGACTATTAC | | RVEAGDEADYYCQVW | | | |
| | | TGTCAGGTGTGGGATGTTGGTGCGGTGTTCGGCGGAGGGACCA | | DVGAVFGGGTKVTVL | | | |
| | | AGGTCACCGTCCTA | | | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81693 | IGKV1-9 | EMTLTQ | 3575 | RASQGI | 3766 | WYQQKP | 2257 | GASTLQ | 2447 | GVPLRF | 4205 | QHLDSY | 2792 | FGQGTK |
| | | SPSYLS | | NNFLA | | GKAPKL | | S | | SGSGSE | | PRYT | | VEIK |
| | | ASAGDR | | | | LIY | | | | TEFTLT | | | | |
| | | VTITC | | | | | | | | ISSLQP | | | | |
| | | | | | | | | | | EDFATY | | | | |
| | | | | | | | | | | YC | | | | |
| ADI-81783 | GKV1-NL1 | DIRMTQ | 3576 | RASQGI | 2062 | WYQQRP | 3944 | GASTLE | 4051 | GVASRF | 4206 | QQYYGP | 4380 | FGQGTK |
| | | SPSSLS | | SNSLA | | GRAPKL | | S | | SGSGSG | | PHT | | VEIK |
| | | TSVGDR | | | | LLY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81823 | IGKV3-20 | EIVMTQ | 1935 | RASQSV | 3767 | WYQQRP | 2306 | GTSNRA | 4065 | GIPDRF | 4207 | QQSVNF | 4381 | FGQGTK |
| | | SPGTLS | | SSGHLA | | GQAPRL | | T | | SGSGSG | | PRT | | VEIK |
| | | LSPGER | | | | LIY | | | | TDFILT | | | | |
| | | ATLSC | | | | | | | | ISRLES | | | | |
| | | | | | | | | | | EDIAVY | | | | |
| | | | | | | | | | | YC | | | | |
| ADI-81888 | IGKV1-NL1 | EIVVTQ | 3577 | RASQGI | 2067 | WYQQKP | 2259 | SASTLE | 4066 | GVPSRF | 4208 | QQYYIL | 4382 | FGQGTK |
| | | SPSSLS | | TNSLA | | GKAPKL | | G | | SGSGSG | | RT | | VEIK |
| | | ASVGDR | | | | LLY | | | | TDFILT | | | | |
| | | VTITC | | | | | | | | IDSLQP | | | | |
| | | | | | | | | | | EDFATY | | | | |
| | | | | | | | | | | YC | | | | |
| ADI-81593 | IGLV1-40 | QSVLTQ | 3578 | TGSNSN | 3768 | WYQKLP | 3945 | GNNNRP | 2492 | GVPDRF | 4209 | QSYDSS | 4383 | FGGGTK |
| | | PPSVSG | | LGAGYD | | GTAPKL | | S | | SGSKSG | | LSSSL | | LTVL |
| | | APVQRV | | VH | | LIY | | | | TSASLA | | | | |
| | | TISC | | | | | | | | IAGLQA | | | | |
| | | | | | | | | | | EDEADY | | | | |
| | | | | | | | | | | FC | | | | |
| ADI-81884 | IGKV1-33 | DITMTQ | 1882 | QASHDI | 3769 | WYQQKP | 2257 | DVLNLK | 4067 | GVPSRF | 4210 | QQYNDF | 4334 | FGQGTK |
| | | SPSSLS | | SDYLN | | GKAPKL | | T | | SGSGSG | | PYT | | VEIK |
| | | ASVGDR | | | | LIY | | | | THFTFT | | | | |
| | | VTITC | | | | | | | | ISSLQP | | | | |
| | | | | | | | | | | EDIATY | | | | |
| | | | | | | | | | | YC | | | | |
| ADI-81545 | IGKV1D-39 | DIQLTQ | 1876 | RASQSI | 3770 | WYQQRP | 2309 | AASTLQ | 2392 | GVPSRF | 4211 | QQSYSS | 4384 | FGPGTK |
| | | SPSSLS | | MRYLN | | GKAPKL | | S | | SGSGSG | | PPWT | | VEIK |
| | | ASVGDR | | | | LIY | | | | TDFRLT | | | | |
| | | VTITC | | | | | | | | ISSLQP | | | | |
| | | | | | | | | | | EDYATY | | | | |
| | | | | | | | | | | YC | | | | |
| ADI-81555 | IGKV1D-39 | EIQLTQ | 3579 | RASQNI | 3771 | WYQQKP | 2257 | AASSLQ | 2391 | GVPSRF | 4212 | QQSYSI | 4385 | FGPGTK |
| | | SPSSLS | | NSYLN | | GKAPKL | | S | | SGSGSK | | PPIT | | VDIK |
| | | ASIGDR | | | | LIY | | | | TNFTLT | | | | |
| | | VIITC | | | | | | | | ITSLQP | | | | |
| | | | | | | | | | | EDFATY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81693 | 2937 | GAAATGACACTGACGCAGTCTCCATCCTACCTGTCTGCATCTG | 4669 | EMTLTQSPSYLSASA | 5023 | 8 | 8 |
| | | CAGGAGACAGAGTCACCATCACTTGCCGGGCCAGTCAGGGCAT | | GDRVTITCRASQGIN | | | |
| | | TAACAATTTTTTAGCCTGGTATCAACAAAAACCAGGGAAAGCC | | NFLAWYQQKPGKAPK | | | |
| | | CCTAAGCTCCTGATCTATGGTGCATCCACTTTGCAAAGTGGGG | | LLIYGASTLQSGVPL | | | |
| | | TCCCATTAAGATTCAGCGGCAGTGGATCTGAGACAGAATTCAC | | RFSGSGSETEFTLTI | | | |
| | | TCTCACAATCAGCAGCCTGCAGCCGGAAGATTTTGCAACTTAT | | SSLQPEDFATYYCQH | | | |
| | | TACTGTCAACACCTTGATAGTTACCCTCGGTACACTTTTGGCC | | LDSYPRYTFGQGTKV | | | |
| | | AGGGGACCAAGGTGGAAATCAAA | | EIK | | | |
| ADI-81783 | 2937 | GACATCCGGATGACCCAGTCTCCATCCTCCCTGTCTACATCTG | 4670 | DIRMTQSPSSLSTSV | 5024 | 8 | 7 |
| | | TAGGAGACCGAGTCACCATCACTTGCCGGGCGAGTCAGGGCAT | | GDRVTITCRASQGIS | | | |
| | | TAGCAATTCTTTAGCCTGGTATCAGCAGAGACCAGGGAGAGCC | | NSLAWYQQRPGRAPK | | | |
| | | CCTAAACTCCTGCTCTATGGTGCATCCACATTGGAAAGTGGGG | | LLLYGASTLESGVAS | | | |
| | | TCGCGTCCAGGTTCAGTGGCAGTGGATCTGGGACGGATTTCAC | | RFSGSGSGTDFTLTI | | | |
| | | TCTCACCATCAGCAGCCTGCAGCCTGAAGATTTTGCAACTTAT | | SSLQPEDFATYYCQQ | | | |
| | | TACTGTCAACAGTACTATGGGCCCCCTCACACTTTTGGCCAGG | | YYGPPHTFGQGTKVE | | | |
| | | GGACCAAGGTGGAGATCAAA | | IK | | | |
| ADI-81823 | 2937 | GAAATTGTGATGACGCAGTCTCCAGGCACCCTGTCTTTGTCTC | 4671 | EIVMTQSPGTLSLSP | 5025 | 8 | 8 |
| | | CAGGGGAAAGAGCCACACTCTCCTGCAGGGCCAGTCAGAGTGT | | GERATLSCRASQSVS | | | |
| | | TAGCAGCGGCCACCTAGCCTGGTACCAGCAGAGACCTGGCCAG | | SGHLAWYQQRPGQAP | | | |
| | | GCTCCCAGGCTCCTCATCTATGGTACATCCAACAGGGCCACTG | | RLLIYGTSNRATGIP | | | |
| | | GCATCCCTGACAGGTTCAGTGGCAGTGGGTCTGGGACAGACTT | | DRFSGSGSGTDFILT | | | |
| | | CATTCTCACCATCAGCAGACTGGAGTCTGAAGATATTGCAGTG | | ISRLESEDIAVYYCQ | | | |
| | | TACTATTGTCAGCAATCCGTTAACTTCCCGAGAACGTTCGGCC | | QSVNFPRTFGQGTKV | | | |
| | | AAGGGACCAAGGTGGAAATCAAA | | EIK | | | |
| ADI-81888 | 2937 | GAAATCGTGGTGACGCAGTCTCCATCCTCCCTGTCTGCATCTG | 4672 | EIVVTQSPSSLSASV | 5026 | 8 | 7 |
| | | TAGGAGACAGAGTCACCATCACTTGCCGGGCGAGTCAGGGCAT | | GDRVTITCRASQGIT | | | |
| | | TACCAATTCCTTAGCCTGGTATCAGCAGAAACCAGGGAAAGCC | | NSLAWYQQKPGKAPK | | | |
| | | CCTAAGCTCCTACTCTATTCTGCATCCACATTGGAAGGTGGGG | | LLLYSASTLEGGVPS | | | |
| | | TCCCATCCAGGTTCAGTGGCAGTGGATCTGGGACGGATTTCAT | | RFSGSGSGTDFILTI | | | |
| | | TCTCACCATCGACAGCCTGCAGCCTGAAGATTTTGCAACTTAT | | DSLQPEDFATYYCQQ | | | |
| | | TACTGTCAACAGTATTATATTCTCCGGACGTTCGGCCAGGGGA | | YYILRTFGQGTKVEI | | | |
| | | CCAAGGTGGAGATCAAA | | K | | | |
| ADI-81593 | 2939 | CAGTCTGTGTTGACGCAGCCGCCCTCAGTGTCTGGGGCCCCAG | 4673 | QSVLTQPPSVSGAPV | 5027 | 8 | 7 |
| | | TGCAGAGAGTCACCATCTCCTGCACTGGGAGCAACTCCAACCT | | QRVTISCTGSNSNLG | | | |
| | | CGGGGCAGGTTATGATGTACACTGGTATCAGAAACTTCCAGGG | | AGYDVHWYQKLPGTA | | | |
| | | ACAGCCCCCAAACTCCTCATCTATGGAAACAACAATCGGCCCT | | PKLLIYGNNNRPSGV | | | |
| | | CAGGGGTCCCTGACCGATTCTCTGGCTCCAAGTCTGGCACCTC | | PDRFSGSKSGTSASL | | | |
| | | AGCCTCCCTGGCCATCGCTGGTCTCCAGGCTGAGGATGAGGCT | | AIAGLQAEDEADYFC | | | |
| | | GATTATTTCTGCCAGTCCTATGACAGCAGCCTGAGTTCTTCGC | | QSYDSSLSSSLFGGG | | | |
| | | TGTTCGGCGGCGGGACCAAGCTCACCGTCCTA | | TKLTVL | | | |
| ADI-81884 | 2937 | GACATCACGATGACCCAGTCTCCATCCTCCCTGTCTGCATCTG | 4674 | DITMTQSPSSLSASV | 5028 | 8 | 6 |
| | | TCGGGGACAGAGTCACCATCACTTGCCAGGCGAGTCACGACAT | | GDRVTITCQASHDIS | | | |
| | | TAGCGACTATTTAAATTGGTATCAGCAAAAACCAGGGAAGGCC | | DYLNWYQQKPGKAPK | | | |
| | | CCTAAGCTCCTGATCTACGATGTGTTAAATTTGAAAACAGGGG | | LLIYDVLNLKTGVPS | | | |
| | | TCCCCTCAAGGTTCAGTGGAAGTGGATCTGGGACACATTTTAC | | RFSGSGSGTHFTFTI | | | |
| | | TTTCACCATCAGCAGCCTGCAGCCTGAAGATATTGCAACATAT | | SSLQPEDIATYYCQQ | | | |
| | | TACTGTCAACAATATAATGATTTCCCGTACACTTTTGGCCAGG | | YNDFPYTFGQGTKVE | | | |
| | | GGACCAAGGTGGAGATCAAA | | IK | | | |
| ADI-81545 | 2942 | GACATCCAGTTGACCCAGTCTCCATCCTCCCTGTCTGCATCTG | 4675 | DIQLTQSPSSLSASV | 5029 | 8 | 6 |
| | | TAGGAGACAGAGTCACCATCACTTGCCGGGCAAGTCAGTCCAT | | GDRVTITCRASQSIM | | | |
| | | TATGAGATACTTAAATTGGTATCAGCAGAGACCAGGGAAGGCC | | RYLNWYQQRPGKAPK | | | |
| | | CCTAAGCTCCTGATCTATGCTGCATCCACTTTGCAAAGTGGGG | | LLIYAASTLQSGVPS | | | |
| | | TCCCATCAAGGTTCAGTGGCAGTGGATCTGGGACAGATTTCAG | | RFSGSGSGTDFRLTI | | | |
| | | GCTCACCATCAGCAGTCTGCAACCTGAAGACTATGCAACTTAT | | SSLQPEDYATYYCQQ | | | |
| | | TACTGTCAACAGAGTTACAGTAGTCCTCCGTGGACGTTCGGCC | | SYSSPPWTFGPGTKV | | | |
| | | CAGGGACCAAAGTGGAAATCAAA | | EIK | | | |
| ADI-81555 | 2950 | GAAATCCAATTGACCCAGTCTCCATCCTCCCTGTCTGCATCTA | 4676 | EIQLTQSPSSLSASI | 5030 | 8 | 7 |
| | | TAGGAGACAGAGTCATCATCACTTGCCGGGCAAGTCAGAACAT | | GDRVIITCRASQNIN | | | |
| | | TAACAGCTATTTAAATTGGTATCAGCAGAAACCAGGGAAAGCC | | SYLNWYQQKPGKAPK | | | |
| | | CCTAAGCTCCTGATCTATGCTGCATCCAGTTTGCAAAGTGGGG | | LLIYAASSLQSGVPS | | | |
| | | TCCCATCAAGGTTCAGTGGCAGTGGATCTAAGACAAATTTCAC | | RFSGSGSKTNFTLTI | | | |
| | | TCTCACCATCACCAGTCTGCAACCTGAAGATTTTGCAACTTAC | | TSLQPEDFATYYCQQ | | | |
| | | TACTGTCAACAGAGTTACAGTATCCCCCCAATCACTTTCGGCC | | SYSIPPITFGPGTKV | | | |
| | | CTGGGACCAAGGTGGATATCAAA | | DIK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81838 | IGLV2-14 | QSVLTQ | 3580 | TATSSD | 3772 | WYQLHP | 3946 | EVSNRP | 2427 | GVFNRF | 4213 | SSYTHD | 4386 | FGGGTK |
| | | PASVYG | | VGSYNY | | GKAPKL | | S | | FGSKSG | | STV | | LTVL |
| | | SPGQSI | | VS | | LIY | | | | NTASLT | | | | |
| | | TISC | | | | | | | | ISGLQA | | | | |
| | | | | | | | | | | EDEADY | | | | |
| | | | | | | | | | | YC | | | | |
| ADI-81623 | IGKV1-16 | EIQLTQ | 1917 | RASQAI | 3773 | WFQQQP | 3947 | AASNLQ | 4068 | GVSSRF | 4214 | QQYKTY | 4387 | FGGGTK |
| | | SPSSLS | | GNYVA | | GKAPKS | | G | | SGSGSG | | PLT | | VEIK |
| | | ASVGDR | | | | LID | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81551 | IGKV3-20 | EIVLTQ | 3581 | RASQSV | 3774 | WYQLKP | 3948 | TVSSRA | 4069 | GIPDRF | 2616 | QQYGTS | 4388 | FGQGTK |
| | | SPGTLS | | RSDYLA | | GQAPRL | | T | | SGSGSG | | PWT | | VEIK |
| | | LSPGER | | | | LIY | | | | TDFTLT | | | | |
| | | ATLSC | | | | | | | | ISRLEP | | | | |
| | | | | | | | | | | EDFAVY | | | | |
| | | | | | | | | | | YC | | | | |
| ADI-81594 | IGKV1-16 | DIQMTQ | 1878 | RASQGI | 3775 | WFQQKP | 3949 | GASNLQ | 4070 | GVSSKF | 4215 | QQYKSF | 4389 | FGGGTK |
| | | SPSSLS | | RTYLA | | GEAPKS | | S | | SGSGSG | | PLT | | VEIK |
| | | ASVGDR | | | | LIE | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81681 | IGLV2-8 | QSVLTQ | 3582 | TGTNSD | 3776 | WYQHHP | 3950 | EVNKRP | 2394 | GVPDRF | 4216 | TSYADT | 4390 | FGGGTK |
| | | PPSASG | | VGGHNS | | GKAPKL | | S | | SGSKSG | | GDCVL | | VTVL |
| | | SPGQSV | | VS | | VIF | | | | NTASLT | | | | |
| | | TISC | | | | | | | | VSGLQA | | | | |
| | | | | | | | | | | DDEADY | | | | |
| | | | | | | | | | | YC | | | | |
| ADI-81833 | IGKV3D-15 | EMTLTQ | 3583 | RASQSV | 3777 | WYQQKP | 2258 | DASTRA | 2424 | GIPARF | 4217 | LQYNNW | 4391 | FGGGTK |
| | | SPATLS | | SRKLA | | GQAPRL | | T | | TGSGSG | | PPLT | | VDIK |
| | | LSPGER | | | | ILIY | | | | TEFTLT | | | | |
| | | ADLSC | | | | | | | | ISSLQS | | | | |
| | | | | | | | | | | EDFAIY | | | | |
| | | | | | | | | | | YC | | | | |
| ADI-81672 | IGLV1-44 | QSKLTQ | 3584 | CGSSSN | 3778 | WYQQLP | 3951 | SNNQRP | 2463 | GVPDRF | 2615 | ATWDDS | 4392 | FGGGTK |
| | | PPSAFG | | LGSNCV | | RTAPKL | | S | | SGSKSG | | VTGPV | | LPVL |
| | | TPGQRV | | N | | LVY | | | | TSASLA | | | | |
| | | TISC | | | | | | | | ISGLQS | | | | |
| | | | | | | | | | | EDEADY | | | | |
| | | | | | | | | | | YC | | | | |
| ADI-81872 | IGKV1-NL1 | DIPMTQ | 1947 | RASQDI | 2113 | WYQQKP | 2296 | AASVLE | 4071 | GVPSRF | 4218 | QHYYSN | 4393 | FGQGTK |
| | | SPSSLS | | SNSLA | | GKAPKF | | S | | SGRGSG | | PRT | | VEIK |
| | | ASVGDR | | | | LLY | | | | TNFTLA | | | | |
| | | VTITC | | | | | | | | ISSLQP | | | | |
| | | | | | | | | | | EDFATY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81838 | 2939 | CAGTCTGTCCTGACTCAGCCTGCCTCCGTGTATGGGTCTCCTG | 4677 | QSVLTQPASVYGSPG | 5031 | 8 | 7 |
| | | GACAGTCGATCACCATCTCCTGCACTGCAACCAGCAGTGACGT | | QSITISCTATSSDVG | | | |
| | | TGGTAGTTATAATTATGTCTCCTGGTACCAACTGCACCCAGGC | | SYNYVSWYQLHPGKA | | | |
| | | AAAGCCCCCAAACTCCTCATTTATGAGGTCAGTAATCGGCCCT | | PKLLIYEVSNRPSGV | | | |
| | | CAGGAGTTTTTAATCGCTTCTTTGGCTCCAAGTCTGGCAACAC | | FNRFFGSKSGNTASL | | | |
| | | GGCCTCCCTGACCATCTCTGGACTTCAGGCTGAGGACGAGGCT | | TISGLQAEDEADYYC | | | |
| | | GATTATTACTGCAGTTCATATACACACGACAGCACGGTCTTCG | | SSYTHDSTVFGGGTK | | | |
| | | GCGGAGGGACCAAGCTCACCGTCCTA | | ILTVL | | | |
| ADI-81623 | 2936 | GAAATCCAGTTGACCCAGTCTCCATCCTCACTGTCTGCATCTG | 4678 | EIQLTQSPSSLSASV | 5032 | 8 | 8 |
| | | TAGGAGACAGGGTCACCATCACTTGTCGGGCGAGTCAGGCCAT | | GDRVTITCRASQAIG | | | |
| | | TGGCAATTATGTAGCCTGGTTTCAGCAGCAACCAGGGAAAGCC | | NYVAWFQQQPGKAPK | | | |
| | | CCTAAGTCCCTTATAGATGCTGCATCAAATTTGCAAGGTGGGG | | SLIDAASNLQGGVSS | | | |
| | | TCTCATCAAGATTCAGCGGCAGTGGATCTGGCACAGATTTCAC | | RFSGSGSGTDFTLTI | | | |
| | | TCTCACCATCAGCAGCCTGCAGCCCGAAGATTTTGCAACTTAT | | SSLQPEDFATYYCQQ | | | |
| | | TACTGCCAACAGTACAAAACTTACCCCCTCACTTTCGGCGGGG | | YKTYPLTFGGGTKVE | | | |
| | | GGACCAAGGTGGAAATCAAA | | IK | | | |
| ADI-81551 | 2937 | GAAATTGTGTTGACACAGTCTCCAGGCACCCTGTCTTTGTCTC | 4679 | EIVLTQSPGTLSLSP | 5033 | 8 | 5 |
| | | CAGGGGAAAGAGCCACCCTGTCCTGCAGGGCCAGTCAGAGTGT | | GERATLSCRASQSVR | | | |
| | | TCGCAGCGACTACTTAGCCTGGTACCAGCTGAAACCTGGCCAG | | SDYLAWYQLKPGQAP | | | |
| | | GCTCCCAGGCTCCTCATTTATACTGTTTCCAGCAGGGCCACTG | | RLLIYTVSSRATGIP | | | |
| | | GCATCCCTGACAGGTTCAGTGGCAGTGGGTCTGGGACAGACTT | | DRFSGSGSGTDFTLT | | | |
| | | CACTCTCACCATCAGCAGACTGGAGCCTGAAGATTTTGCAGTG | | ISRLEPEDFAVYYCQ | | | |
| | | TATTACTGTCAGCAGTATGGTACCTCACCCTGGACGTTCGGCC | | QYGTSPWTFGQGTKV | | | |
| | | AAGGGACCAAGGTGGAAATCAAA | | EIK | | | |
| ADI-81594 | 2936 | GACATCCAGATGACCCAGTCTCCATCCTCACTGTCTGCATCTG | 4680 | DIQMTQSPSSLSASV | 5034 | 8 | 7 |
| | | TAGGAGACAGAGTCACCATCACTTGTCGGGCGAGTCAGGGCAT | | GDRVTITCRASQGIR | | | |
| | | TAGGACTTATTTAGCCTGGTTTCAGCAGAAACCAGGCGAAGCC | | TYLAWFQQKPGEAPK | | | |
| | | CCTAAGTCCCTGATCGAGGGTGCATCAAATTTGCAAAGTGGGG | | SLIEGASNLQSGVSS | | | |
| | | TCTCATCAAAATTCAGCGGCAGTGGATCTGGCACAGATTTCAC | | KFSGSGSGTDFTLTI | | | |
| | | TCTCACCATCAGCAGCCTGCAGCCTGAAGATTTTGCAACTTAC | | SSLQPEDFATYYCQQ | | | |
| | | TACTGCCAACAGTATAAAAGTTTCCCCCTCACTTTCGGCGGAG | | YKSFPLTFGGGTKVE | | | |
| | | GGACCAAAGTGGAAATCAAA | | IK | | | |
| ADI-81681 | 2947 | CAGTCTGTGCTGACTCAGCCTCCCTCCGCGTCCGGGTCTCCTG | 4681 | QSVLTQPPSASGSPG | 5035 | 8 | 8 |
| | | GACAGTCAGTCACCATCTCCTGCACTGGAACCAACAGTGACGT | | QSVTISCTGTNSDVG | | | |
| | | TGGTGGTCATAACTCTGTCTCCTGGTACCAACACCACCCAGGC | | GHNSVSWYQHHPGKA | | | |
| | | AAAGCCCCCAAACTCGTGATTTTTGAGGTCAATAAGCGGCCCT | | PKLVIFEVNKRPSGV | | | |
| | | CAGGGGTCCCTGATCGCTTCTCTGGCTCCAAGTCTGGCAACAC | | PDRFSGSKSGNTASL | | | |
| | | GGCCTCCCTGACCGTCTCTGGGCTCCAGGCTGACGATGAGGCT | | TVSGLQADDEADYYC | | | |
| | | GATTATTACTGCACCTCATATGCAGACACCGGCGATTGTGTGC | | TSYADTGDCVLFGGG | | | |
| | | TTTTCGGCGGAGGGACCAAGGTCACCGTCCTA | | TKVTVL | | | |
| ADI-81833 | 2938 | GAAATGACACTCACGCAGTCTCCAGCCACCCTGTCTTTGTCTC | 4682 | EMTLTQSPATLSLSP | 5036 | 8 | 7 |
| | | CAGGTGAAAGAGCCGACCTCTCTTGCAGGGCCAGTCAGAGCGT | | GERADLSCRASQSVS | | | |
| | | TAGCCGCAAGTTAGCCTGGTATCAACAGAAACCTGGCCAGGCT | | RKLAWYQQKPGQAPR | | | |
| | | CCCAGGCTCCTCATTTATGATGCATCCACCAGGGCCACTGGCA | | LLIYDASTRATGIPA | | | |
| | | TCCCAGCCAGGTTCACTGGCAGTGGGTCTGGGACAGAGTTCAC | | RFTGSGSGTEFTLTI | | | |
| | | TCTCACCATCAGCAGCCTGCAGTCTGAAGATTTTGCAATTTAT | | SSLQSEDFAIYYCLQ | | | |
| | | TACTGTCTGCAGTATAATAATTGGCCTCCCCTCACTTTCGGCG | | YNNWPPLTFGGGTKV | | | |
| | | GAGGGACCAAGGTGGATATCAAA | | DIK | | | |
| ADI-81672 | 4595 | CAGTCAAAACTGACTCAGCCACCCTCAGCGTTTGGGACCCCCG | 4683 | QSKLTQPPSAFGTPG | 5037 | 8 | 6 |
| | | GGCAGAGGGTCACCATCTCTTGTTGTGGAAGCAGCTCCAACCT | | QRVTISCCGSSSNLG | | | |
| | | TGGAAGTAATTGTGTAAACTGGTACCAGCAACTCCCAAGAACG | | SNCVNWYQQLPRTAP | | | |
| | | GCCCCCAAACTCCTCGTCTATAGTAATAATCAGCGGCCCTCAG | | KLLVYSNNQRPSGVP | | | |
| | | GGGTCCCTGACCGATTCTCTGGCTCCAAGTCCGGCACCTCAGC | | DRFSGSKSGTSASLA | | | |
| | | CTCCCTGGCCATCAGTGGGCTCCAGTCTGAGGATGAGGCTGAT | | ISGLQSEDEADYYCA | | | |
| | | TATTACTGCGCAACATGGGATGACAGCGTGACTGGTCCGGTGT | | TWDDSVTGPVFGGGT | | | |
| | | TCGGCGGAGGGACCAAGCTCCCCGTCTTA | | KLPVL | | | |
| ADI-81872 | 2937 | GACATCCCAATGACCCAGTCTCCATCCTCCCTGTCTGCATCTG | 4684 | DIPMTQSPSSLSASV | 5038 | 8 | 7 |
| | | TTGGAGACAGAGTCACCATCACTTGCCGGGCGAGTCAGGACAT | | GDRVTITCRASQDIS | | | |
| | | TAGCAATTCTTTAGCCTGGTATCAGCAGAAACCAGGGAAAGCC | | NSLAWYQQKPGKAPK | | | |
| | | CCTAAGTTCCTCCTCTATGCTGCATCCGTATTGGAAAGTGGGG | | FLLYAASVLESGVPS | | | |
| | | TCCCATCCAGGTTCAGTGGCCGTGGATCTGGGACCAATTTCAC | | RFSGRGSGTNFTLAI | | | |
| | | TCTCGCCATCAGCAGCCTGCAGCCTGAAGATTTTGCAACTTAT | | SSLQPEDFATYYCQH | | | |
| | | TACTGTCAACATTATTATAGTAATCCTCGGACTTTTGGCCAGG | | YYSNPRTFGQGTKVE | | | |
| | | GGACCAAGGTGGAGATCAAA | | IK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81679 | IGKV1D-39 | DIQMTQ | 3585 | RASQSI | 3738 | WYQHKP | 3952 | AASSLQ | 2391 | GVPSRF | 2534 | QQSYTL | 4394 | FGGGTK |
| | | SPSSLS | | NNYLN | | GRAPKL | | S | | SGSGSG | | PGAT | | VEIK |
| | | AFVGDR | | | | LIY | | | | TDFTLT | | | | |
| | | ISITC | | | | | | | | ISSLQP | | | | |
| | | | | | | | | | | EDFATY | | | | |
| | | | | | | | | | | YC | | | | |
| ADI-81553 | IGKV3D-15 | EIVMTQ | 2034 | RASQSI | 2129 | WYQQKF | 3953 | GISTGA | 4072 | GTPARF | 4219 | QQYNHW | 4395 | FGQGTK |
| | | SPATLS | | SSNLA | | GQAPRL | | T | | RGSGSG | | PGT | | VEIK |
| | | VSPGER | | | | LIY | | | | TEFTLT | | | | |
| | | ATLSC | | | | | | | | ISSLQS | | | | |
| | | | | | | | | | | EDFAVY | | | | |
| | | | | | | | | | | YC | | | | |
| ADI-81636 | IGLV1-40 | QSVLTQ | 3586 | TGSSSN | 2201 | WYQQLP | 2314 | SNNNRP | 4073 | GVPDRI | 4220 | QSYDSN | 4396 | LGEGTK |
| | | PPSVSG | | IGAGYD | | GTAPKL | | S | | SGSRSD | | VFGKV | | LTVL |
| | | APGHRV | | VH | | LIY | | | | TSASLA | | | | |
| | | TISC | | | | | | | | ITGLQA | | | | |
| | | | | | | | | | | EDEADY | | | | |
| | | | | | | | | | | FC | | | | |
| ADI-81536 | IGLV2-14 | QPVLTQ | 1933 | TGTSSD | 3779 | WYQQHP | 3954 | EFSNRP | 4074 | GVSHRF | 4221 | SSYTTD | 4397 | FGGGTK |
| | | PASVSG | | LGNFNY | | GKGPKL | | S | | SGSKSG | | SVV | | VTVL |
| | | SPGQSI | | VS | | MIY | | | | NTASLT | | | | |
| | | TISC | | | | | | | | ISGLQP | | | | |
| | | | | | | | | | | EDEADY | | | | |
| | | | | | | | | | | YC | | | | |
| ADI-81684 | IGKV1D-39 | DIAMTQ | 3587 | RASQSI | 3780 | WYQQKP | 3942 | GASTLQ | 2447 | GVPSRF | 4222 | QQSYSS | 4398 | FGQGTK |
| | | SPSSLS | | FSYLN | | GKAPKL | | S | | SGSGSG | | MWT | | VEIK |
| | | ASVGDR | | | | LIF | | | | TDFTLT | | | | |
| | | VTISC | | | | | | | | ISSLQP | | | | |
| | | | | | | | | | | EDLATY | | | | |
| | | | | | | | | | | SC | | | | |
| ADI-81794 | IGLV1-44 | QSELTQ | 3588 | SGSSSN | 3781 | WYQQLP | 3955 | NNNQRP | 4061 | GVPDRF | 4223 | AAWDDS | 4399 | FGGGTK |
| | | PPSTSG | | IGGNVV | | GTTPKV | | S | | SGSKSG | | LNGPLV | | LTVL |
| | | TPGQRV | | N | | LIY | | | | TTASLA | | | | |
| | | TISC | | | | | | | | ISGLOS | | | | |
| | | | | | | | | | | EDEADY | | | | |
| | | | | | | | | | | YC | | | | |
| ADI-81869 | IGKV1D-39 | EMTITQ | 3589 | RASQSI | 3782 | WYQHKP | 3924 | DSSSLQ | 4075 | GVPSRF | 2534 | QQSYST | 2741 | FGQGTK |
| | | SPSSLS | | RGHLN | | GKAPKL | | S | | SGSGSG | | PPWT | | VEIK |
| | | ASVGDR | | | | LIY | | | | TDFTLT | | | | |
| | | ITITC | | | | | | | | ISSLQP | | | | |
| | | | | | | | | | | EDFATY | | | | |
| | | | | | | | | | | YC | | | | |
| ADI-81659 | IGLV1-40 | QSVLTQ | 1948 | TGSNSN | 2143 | WYRQLP | 3956 | GNTNRP | 2484 | GVPDRF | 4224 | QSYDSS | 4400 | FGGGTK |
| | | PPSVSG | | IGAGYD | | GTAPQL | | S | | SASKSG | | LSGVI | | VTVL |
| | | APGQRV | | VH | | IFY | | | | TSASLA | | | | |
| | | TISC | | | | | | | | ITGLQA | | | | |
| | | | | | | | | | | DDEADY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81679 | 2936 | GACATCCAGATGACCCAGTCTCCATCCTCCCTGTCTGCATTTG | 4685 | DIQMTQSPSSLSAFV | 5039 | 8 | 7 |
| | | TAGGAGACAGAATCAGCATCACTTGCCGGGCAAGTCAGAGCAT | | GDRISITCRASQSIN | | | |
| | | TAACAATTACCTAAATTGGTATCAGCACAAACCGGGGAGAGCC | | NYLNWYQHKPGRAPK | | | |
| | | CCTAAGCTCCTGATCTATGCTGCATCCAGTTTGCAAAGTGGGG | | LLIYAASSLQSGVPS | | | |
| | | TCCCATCAAGGTTCAGTGGTAGTGGATCTGGGACAGATTTCAC | | RFSGSGSGTDFTLTI | | | |
| | | TCTCACCATCAGCAGTCTGCAACCTGAAGATTTTGCAACTTAT | | SSLQPEDFATYYCQQ | | | |
| | | TACTGTCAACAGAGTTACACTCTCCCTGGAGCCACTTTCGGCG | | SYTLPGATFGGGTKV | | | |
| | | GAGGGACCAAGGTGGAGATCAAA | | EIK | | | |
| ADI-81553 | 2937 | GAAATTGTGATGACCCAGTCTCCAGCCACCCTGTCTGTGTCTC | 4686 | EIVMTQSPATLSVSP | 5040 | 8 | 6 |
| | | CAGGGGAAAGAGCCACCCTCTCCTGCAGGGCCAGTCAGAGTAT | | GERATLSCRASQSIS | | | |
| | | TAGCAGCAACTTAGCCTGGTACCAGCAGAAATTTGGCCAGGCT | | SNLAWYQQKFGQAPR | | | |
| | | CCCAGGCTCCTCATCTATGGTATATCCACCGGGGCCACTGGTA | | LLIYGISTGATGTPA | | | |
| | | CCCCAGCCAGGTTCAGAGGCAGTGGGTCTGGGACAGAGTTCAC | | RFRGSGSGTEFTLTI | | | |
| | | TCTCACCATCAGCAGCCTGCAGTCTGAAGATTTTGCAGTTTAT | | SSLQSEDFAVYYCQQ | | | |
| | | TACTGTCAGCAGTATAATCACTGGCCGGGCACGTTCGGCCAAG | | YNHWPGTFGQGTKVE | | | |
| | | GGACCAAGGTGGAAATCAAA | | IK | | | |
| ADI-81636 | 4596 | CAGTCTGTCTTGACGCAGCCGCCCTCAGTGTCTGGGGCCCCAG | 4687 | QSVLTQPPSVSGAPG | 5041 | 8 | 7 |
| | | GACATAGGGTCACCATCTCCTGCACTGGGAGCAGCTCCAACAT | | HRVTISCTGSSSNIG | | | |
| | | CGGGGCAGGTTATGATGTGCACTGGTACCAGCAGCTTCCAGGA | | AGYDVHWYQQLPGTA | | | |
| | | ACAGCCCCCAAACTCCTCATCTATAGTAATAATAATCGGCCCT | | PKLLIYSNNNRPSGV | | | |
| | | CAGGGGTCCCTGACCGAATCTCTGGCTCCAGGTCTGACACCTC | | PDRISGSRSDTSASL | | | |
| | | AGCCTCCCTGGCCATCACTGGACTCCAGGCTGAGGATGAGGCT | | AITGLQAEDEADYFC | | | |
| | | GATTATTTCTGCCAGTCCTATGACAGTAACGTGTTCGGTAAGG | | QSYDSNVFGKVLGEG | | | |
| | | TATTGGGCGAAGGGACCAAGCTGACCGTCCTA | | TKLTVL | | | |
| ADI-81536 | 2947 | CAGCCTGTGCTGACTCAGCCTGCCTCCGTGTCTGGGTCTCCTG | 4688 | QPVLTQPASVSGSPG | 5042 | 8 | 7 |
| | | GACAGTCGATCACCATCTCCTGCACTGGAACCAGCAGTGACCT | | QSITISCTGTSSDLG | | | |
| | | TGGTAATTTTAACTATGTCTCCTGGTACCAACAACACCCAGGC | | NFNYVSWYQQHPGKG | | | |
| | | AAGGGCCCCAAACTCATGATTTATGAGTTCAGTAATCGGCCCT | | PKLMIYEFSNRPSGV | | | |
| | | CAGGGGTTTCTCATCGCTTCTCTGGCTCCAAGTCTGGCAACAC | | SHRFSGSKSGNTASL | | | |
| | | GGCCTCCCTGACCATCTCTGGGCTCCAGCCTGAGGACGAGGCT | | TISGLQPEDEADYYC | | | |
| | | GATTATTACTGCAGCTCATATACAACCGACAGCGTCGTTTTCG | | SSYTTDSVVFGGGTK | | | |
| | | GCGGAGGGACCAAGGTCACCGTCCTA | | VTVL | | | |
| ADI-81684 | 2937 | GACATCGCGATGACCCAGTCTCCATCCTCCCTGTCTGCATCTG | 4689 | DIAMTQSPSSLSASV | 5043 | 8 | 7 |
| | | TAGGAGACAGAGTCACCATCAGTTGTCGGGCAAGTCAGAGTAT | | GDRVTISCRASQSIF | | | |
| | | TTTCAGCTATTTAAATTGGTATCAACAGAAACCAGGGAAAGCC | | SYLNWYQQKPGKAPK | | | |
| | | CCTAAGCTCCTGATCTTTGGTGCATCCACTTTGCAAAGTGGGG | | LLIFGASTLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAACCTGAAGATCTTGCAACTTAC | | SSLQPEDLATYSCQQ | | | |
| | | TCCTGTCAACAGAGTTACAGTTCAATGTGGACGTTCGGCCAAG | | SYSSMWTFGQGTKVE | | | |
| | | GGACCAAGGTGGAAATCAAA | | IK | | | |
| ADI-81794 | 2939 | CAGTCTGAGCTGACTCAGCCACCCTCAACGTCTGGGACCCCCG | 4690 | QSELTQPPSTSGTPG | 5044 | 8 | 7 |
| | | GGCAGAGGGTCACCATCTCTTGTTCTGGAAGCAGCTCCAACAT | | QRVTISCSGSSSNIG | | | |
| | | CGGAGGTAATGTTGTGAACTGGTACCAGCAGCTGCCAGGAACG | | GNVVNWYQQLPGTTP | | | |
| | | ACCCCCAAAGTCCTCATCTATAATAATAATCAGCGGCCCTCAG | | KVLIYNNNQRPSGVP | | | |
| | | GGGTCCCTGACCGATTCTCTGGCTCCAAGTCTGGCACCACAGC | | DRFSGSKSGTTASLA | | | |
| | | CTCCCTGGCCATCAGTGGGCTCCAGTCTGAGGATGAGGCTGAT | | ISGLQSEDEADYYCA | | | |
| | | TATTACTGTGCAGCATGGGATGACAGCCTGAATGGCCCCTTGG | | AWDDSLNGPLVFGGG | | | |
| | | TATTCGGCGGAGGGACCAAGCTCACCGTCCTA | | TKLTVL | | | |
| ADI-81869 | 2937 | GAAATGACGATCACGCAGTCTCCATCCTCCCTGTCTGCATCTG | 4691 | EMTITQSPSSLSASV | 5045 | 8 | 7 |
| | | TAGGAGACAGAATCACCATCACTTGCCGGGCAAGTCAGAGCAT | | GDRITITCRASQSIR | | | |
| | | TAGAGGACATTTAAACTGGTATCAGCACAAACCAGGGAAAGCC | | GHLNWYQHKPGKAPK | | | |
| | | CCTAAGCTCCTGATCTATGATTCATCCAGTTTGCAAAGTGGGG | | LLIYDSSSLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAGCCTGAAGATTTTGCAACTTAC | | SSLQPEDFATYYCQQ | | | |
| | | TACTGTCAACAGAGTTACAGTACCCCTCCGTGGACGTTCGGCC | | SYSTPPWTFGQGTKV | | | |
| | | AAGGGACCAAGGTGGAGATCAAA | | EIK | | | |
| ADI-81659 | 2947 | CAGTCTGTCTTGACGCAGCCGCCCTCAGTGTCTGGGGCCCCAG | 4692 | QSVLTQPPSVSGAPG | 5046 | 8 | 8 |
| | | GGCAGAGGGTCACCATCTCCTGCACTGGGAGCAACTCCAACAT | | QRVTISCTGSNSNIG | | | |
| | | CGGGGCAGGTTATGATGTACACTGGTACCGGCAACTTCCTGGA | | AGYDVHWYRQLPGTA | | | |
| | | ACAGCCCCCCAACTCATCTTCTACGGGAACACCAATCGGCCCT | | PQLIFYGNTNRPSGV | | | |
| | | CAGGGGTCCCTGACCGATTCTCTGCCTCCAAGTCTGGCACTTC | | PDRFSASKSGTSASL | | | |
| | | AGCCTCCCTGGCCATCACTGGGCTCCAGGCTGACGATGAGGCT | | AITGLQADDEADYYC | | | |
| | | GATTATTACTGCCAGTCCTATGACAGCAGCCTGAGTGGTGTGA | | QSYDSSLSGVIFGGG | | | |
| | | TATTCGGCGGAGGGACCAAGGTCACCGTCCTA | | TKVTVL | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81861 | IGKV1-5 | EIVMTQ | 3590 | RARQSI | 3783 | WYQKKP | 3957 | KVSTLE | 4076 | GVPSRF | 2532 | QQSSSY | 4401 | FGQGTK |
| | | SPSTLS | | SNWLA | | GKAPTL | | S | | SGSGSG | | YT | | VDIK |
| | | ASVGDR | | | | LIY | | | | TEFTLT | | | | |
| | | VTITC | | | | | | | | ISSLQP | | | | |
| | | | | | | | | | | DDFATY | | | | |
| | | | | | | | | | | YC | | | | |
| ADI-81707 | IGKV1D-39 | DIQMTQ | 1878 | RASQTI | 3784 | WYQQRP | 2309 | GASTLQ | 2447 | GVPSRF | 4225 | QQSYSS | 4384 | FGPGTK |
| | | SPSSLS | | SRYLN | | GKAPKL | | S | | SGTGSG | | PPWT | | VDIK |
| | | ASVGDR | | | | LIY | | | | TDFTLT | | | | |
| | | VTITC | | | | | | | | IGSLQP | | | | |
| | | | | | | | | | | EDFATY | | | | |
| | | | | | | | | | | YC | | | | |
| ADI-81584 | IGKV1-12 | DIVLTQ | 3591 | RASQDI | 3785 | WYQQRP | 2278 | AASSLE | 4077 | GVPSRF | 4226 | QQAHSF | 4402 | FGQGTK |
| | | SPSSVS | | SSYLA | | GKAPKL | | S | | SGSGSR | | PWT | | VDIK |
| | | ASVGDR | | | | LIF | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81717 | IGKV1D-39 | EIPLTQ | 3592 | RASQSI | 3751 | WYQQKP | 2257 | GASSLQ | 2403 | GVPSRF | 4227 | QQSYGP | 4403 | FGQGTR |
| | | SPSSLS | | DNYLN | | GKAPKL | | S | | SGSGSG | | PPTT | | LEIK |
| | | ASVGDR | | | | LIY | | | | TDFTLT | | | | |
| | | VTLTC | | | | | | | | ISSLQP | | | | |
| | | | | | | | | | | EDFATY | | | | |
| | | | | | | | | | | LC | | | | |
| ADI-81726 | IGKV1D-39 | DIGMTQ | 3564 | RASQTI | 3735 | WYQQKP | 3939 | AASSLQ | 2391 | GVPSRF | 4228 | QQTHSP | 4404 | FGQGTK |
| | | SPSSLS | | NSYLN | | GTAPKL | | S | | SGSGSE | | PRA | | VEIK |
| | | ASVGDR | | | | LIY | | | | THFTLT | | | | |
| | | VTITC | | | | | | | | ITNLQP | | | | |
| | | | | | | | | | | EDFATY | | | | |
| | | | | | | | | | | YC | | | | |
| ADI-81897 | IGKV3-11 | EIVMTQ | 3593 | RASQSF | 3786 | WYQQKP | 2258 | DASNRA | 2448 | GIPARF | 4229 | QHRSN | 4405 | FGQGTR |
| | | SPATLS | | NNYLA | | GQAPRL | | T | | SGSGSG | | | | LELK |
| | | LSPGDR | | | | LIY | | | | TDFTLT | | | | |
| | | ATLSC | | | | | | | | ISSVEP | | | | |
| | | | | | | | | | | EDFAIY | | | | |
| | | | | | | | | | | LC | | | | |
| ADI-81718 | IGKV1D-39 | EIPMTQ | 1912 | RASQSI | 3787 | WYQQKA | 3958 | GASSLQ | 2403 | GVPARF | 4230 | QQSYDY | 4406 | FGQGTK |
| | | SPSSLS | | SGYLN | | GKAPKL | | S | | SGSGSG | | PRT | | VDIK |
| | | ASVGDR | | | | LIY | | | | TDFTLT | | | | |
| | | VTITC | | | | | | | | INTLQP | | | | |
| | | | | | | | | | | DDFATY | | | | |
| | | | | | | | | | | YC | | | | |
| ADI-81770 | IGKV1D-39 | EIVMTQ | 1891 | RASQSI | 3788 | WYQQKP | 2257 | STSNLQ | 4078 | GVPSRF | 2534 | QESYTT | 4407 | FGQGTK |
| | | SPSSLS | | RDFLN | | GKAPKL | | S | | SGSGSG | | PPA | | VEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81861 | 2944 | GAAATCGTGATGACGCAGTCTCCTTCCACCCTGTCTGCATCTG | 4693 | EIVMTQSPSTLSASV | 5047 | 7 | 6 |
| | | TAGGAGACAGAGTCACCATCACTTGCCGGGCCCGTCAGAGTAT | | GDRVTITCRARQSIS | | | |
| | | TAGTAACTGGTTGGCCTGGTATCAGAAGAAACCAGGGAAAGCC | | NWLAWYQKKPGKAPT | | | |
| | | CCTACGCTCCTAATCTATAAGGTATCTACTTTAGAAAGTGGGG | | LLIYKVSTLESGVPS | | | |
| | | TCCCATCAAGGTTCAGCGGCAGTGGATCTGGGACAGAATTCAC | | RFSGSGSGTEFTLTI | | | |
| | | TCTCACCATCAGCAGCCTGCAGCCTGATGATTTTGCAACTTAT | | SSLQPDDFATYYCQQ | | | |
| | | TACTGCCAACAGTCTAGTAGTTATTACACTTTTGGCCAGGGGA | | SSSYYTFGQGTKVDI | | | |
| | | CCAAAGTGGATATCAAA | | K | | | |
| ADI-81707 | 2950 | GACATCCAGATGACCCAGTCTCCATCCTCCCTGTCTGCATCTG | 4694 | DIQMTQSPSSLSASV | 5048 | 7 | 7 |
| | | TAGGAGACAGAGTCACCATCACTTGCCGGGCAAGTCAGACCAT | | GDRVTITCRASQTIS | | | |
| | | TAGTCGCTATTTAAATTGGTATCAGCAGAGACCAGGGAAAGCC | | RYLNWYQQRPGKAPK | | | |
| | | CCTAAGCTCCTGATCTATGGTGCATCCACTTTGCAAAGTGGGG | | LLIYGASTLQSGVPS | | | |
| | | TCCCATCAAGGTTCAGTGGCACTGGATCTGGGACAGATTTCAC | | RFSGTGSGTDFTLTI | | | |
| | | TCTCACCATTGGCAGTCTGCAACCTGAAGATTTTGCAACTTAC | | GSLQPEDFATYYCQQ | | | |
| | | TACTGTCAACAGAGTTACAGCAGTCCACCTTGGACATTCGGCC | | SYSSPPWTFGPGTKV | | | |
| | | CAGGGACCAAGGTGGATATCAAA | | DIK | | | |
| ADI-81584 | 2944 | GACATCGTGCTGACCCAGTCTCCATCTTCCGTGTCCGCCTCTG | 4695 | DIVLTQSPSSVSASV | 5049 | 7 | 6 |
| | | TAGGAGACAGAGTCACCATCACTTGTCGGGCGAGTCAGGATAT | | GDRVTITCRASQDIS | | | |
| | | TAGCAGTTACTTAGCCTGGTATCAGCAGAGACCAGGGAAAGCC | | SYLAWYQQRPGKAPK | | | |
| | | CCTAAGCTCCTGATCTTTGCTGCATCCAGTTTGGAAAGTGGGG | | LLIFAASSLESGVPS | | | |
| | | TCCCATCAAGGTTCAGCGGCAGTGGATCTAGGACAGATTTCAC | | RFSGSGSRTDFTLTI | | | |
| | | TCTCACCATCAGCAGCCTGCAGCCTGAAGACTTTGCGACTTAT | | SSLQPEDFATYYCQQ | | | |
| | | TATTGTCAACAGGCTCACAGTTTCCCGTGGACGTTCGGCCAAG | | AHSFPWTFGQGTKVD | | | |
| | | GGACCAAAGTGGATATCAAA | | IK | | | |
| ADI-81717 | 2940 | GAAATCCCATTGACCCAGTCTCCATCCTCCCTGTCTGCATCTG | 4696 | EIPLTQSPSSLSASV | 5050 | 7 | 5 |
| | | TAGGAGACAGAGTCACCCTCACTTGCCGGGCAAGTCAGAGCAT | | GDRVTLTCRASQSID | | | |
| | | TGACAACTATTTAAATTGGTATCAACAGAAACCAGGGAAAGCC | | NYLNWYQQKPGKAPK | | | |
| | | CCTAAACTCCTGATATATGGTGCATCCAGTTTGCAAAGTGGGG | | LLIYGASSLQSGVPS | | | |
| | | TCCCATCAAGGTTCAGTGGCAGTGGATCTGGGACAGACTTCAC | | RFSGSGSGTDFTLTI | | | |
| | | TCTCACCATCAGCAGTCTGCAACCTGAAGATTTTGCAACCTAC | | SSLQPEDFATYLCQQ | | | |
| | | CTCTGTCAACAGAGTTACGGTCCCCCTCCGACCACCTTCGGCC | | SYGPPPTTFGQGTRL | | | |
| | | AGGGGACACGACTGGAGATTAAA | | EIK | | | |
| ADI-81726 | 2937 | GACATCGGGATGACCCAGTCTCCATCCTCCCTGTCTGCATCTG | 4697 | DIGMTQSPSSLSASV | 5051 | 7 | 7 |
| | | TTGGAGACAGAGTCACCATCACTTGTCGGGCAAGTCAGACCAT | | GDRVTITCRASQTIN | | | |
| | | TAACAGCTATTTAAATTGGTATCAGCAAAAACCAGGGACAGCC | | SYLNWYQQKPGTAPK | | | |
| | | CCTAAACTCCTGATCTATGCTGCATCCAGTTTGCAAAGTGGGG | | LLIYAASSLQSGVPS | | | |
| | | TCCCATCAAGGTTCAGTGGCAGTGGATCTGAGACACATTTCAC | | RFSGSGSETHFTLTI | | | |
| | | TCTCACCATCACCAATCTGCAACCTGAAGATTTTGCAACTTAC | | TNLQPEDFATYYCQQ | | | |
| | | TACTGTCAACAGACTCACAGTCCCCCTCGGGCGTTCGGCCAAG | | THSPPRAFGQGTKVE | | | |
| | | GGACCAAGGTGGAGATCAAA | | IK | | | |
| ADI-81897 | 4597 | GAAATTGTGATGACACAGTCTCCAGCCACCCTGTCTTTGTCTC | 4698 | EIVMTQSPATLSLSP | 5052 | 7 | 6 |
| | | CAGGGGACAGAGCCACCCTCTCCTGCAGGGCCAGTCAGAGTTT | | GDRATLSCRASQSFN | | | |
| | | TAATAACTACTTAGCCTGGTACCAACAGAAACCTGGCCAGGCT | | NYLAWYQQKPGQAPR | | | |
| | | CCCAGGCTCCTCATCTATGATGCATCCAACAGGGCCACTGGCA | | LLIYDASNRATGIPA | | | |
| | | TCCCAGCCAGGTTCAGTGGCAGTGGGTCTGGGACAGACTTCAC | | RFSGSGSGTDFTLTI | | | |
| | | TCTCACCATCAGCAGCGTAGAGCCTGAAGATTTTGCAATTTAT | | SSVEPEDFAIYYCQH | | | |
| | | TACTGTCAACATCGTAGCAACTTCGGCCAAGGGACACGACTGG | | RSNFGQGTRLELK | | | |
| | | AGCTTAAA | | | | | |
| ADI-81718 | 2944 | GAAATCCCGATGACCCAGTCTCCATCCTCCCTGTCTGCATCTG | 4699 | EIPMTQSPSSLSASV | 5053 | 7 | 7 |
| | | TAGGAGACAGAGTCACCATCACTTGCCGGGCAAGTCAGAGCAT | | GDRVTITCRASQSIS | | | |
| | | CAGCGGCTATTTAAATTGGTATCAACAAAAAGCAGGCAAAGCC | | GYLNWYQQKAGKAPK | | | |
| | | CCTAAACTCCTGATCTATGGTGCATCAAGTTTGCAAAGTGGGG | | LLIYGASSLQSGVPA | | | |
| | | TCCCAGCAAGGTTCAGTGGCAGTGGATCTGGGACAGATTTCAC | | RFSGSGSGTDFTLTI | | | |
| | | TCTCACCATCAACACTCTGCAACCTGACGACTTTGCAACTTAT | | NTLQPDDFATYYCQQ | | | |
| | | TACTGTCAACAGAGTTACGATTATCCGCGGACGTTCGGCCAAG | | SYDYPRTFGQGTKVD | | | |
| | | GGACCAAAGTGGATATCAAA | | IK | | | |
| ADI-81770 | 2937 | GAAATCGTGATGACCCAGTCTCCATCGTCCCTGTCTGCATCTG | 4700 | EIVMTQSPSSLSASV | 5054 | 7 | 6 |
| | | TGGGAGACAGGGTCACCATCACTTGCCGGGCAAGTCAGAGCAT | | GDRVTITCRASQSIR | | | |
| | | TCGCGACTTTTTAAATTGGTATCAGCAGAAACCAGGGAAAGCC | | DFLNWYQQKPGKAPK | | | |
| | | CCTAAACTCCTAATCTATTCTACATCCAATTTGCAAAGTGGGG | | LLIYSTSNLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAACCTGAAGATTTCGCAACTTAC | | SSLQPEDFATYYCQE | | | |
| | | TACTGTCAAGAGAGCTACACAACCCCTCCGGCTTTTGGGCAGG | | SYTTPPAFGQGTKVE | | | |
| | | GGACCAAAGTGGAAATCAAA | | IK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81772 | IGLV1-44 | QSELTQ | 3594 | SGTNSN | 3789 | WYQQLP | 2314 | NNYQRP | 4079 | GVPDRF | 4231 | ATWDDS | 4408 | FGTGTK |
| | | PPSASG | | IGGNTV | | GTAPKL | | S | | SGSKSG | | LDGHNV | | LTVL |
| | | TPGQRV | | N | | LIY | | | | TSASLA | | | | |
| | | SISC | | | | | | | | ISGLQS | | | | |
| | | | | | | | | | | EDEAEY | | | | |
| | | | | | | | | | | YC | | | | |
| ADI-81834 | IGLV1-40 | QFVVTQ | 3595 | TGSNSN | 2143 | WYQQLP | 2314 | DNNNRP | 2476 | GVPDRF | 4232 | QSYDSS | 2840 | FGGGTQ |
| | | PPSVFG | | IGAGYD | | GTAPKL | | S | | SGSKFD | | LSDSV | | LTVL |
| | | APGQRV | | VH | | LIY | | | | TSASLA | | | | |
| | | TISC | | | | | | | | ITGLQA | | | | |
| | | | | | | | | | | EDEADY | | | | |
| | | | | | | | | | | YC | | | | |
| ADI-81757 | IGKV1-5 | EITLTQ | 3596 | RASQSI | 2052 | WYQQKP | 3959 | KASSLQ | 4080 | GVPARF | 4233 | QQYDTY | 4409 | FGQGTK |
| | | SPSTLS | | SRWLA | | GKSPNL | | S | | SGSASG | | PWT | | VEIK |
| | | ASVGDR | | | | LIY | | | | TEFTLT | | | | |
| | | VTITC | | | | | | | | ISRLQP | | | | |
| | | | | | | | | | | DDFATY | | | | |
| | | | | | | | | | | YC | | | | |
| ADI-81835 | IGKV1-9 | EIPMTQ | 3597 | RASQGI | 3790 | WYQQKP | 2333 | GVSTLQ | 4081 | GVPLRF | 2595 | QYLDTY | 4410 | FGQGTK |
| | | SPSFLS | | PSFLA | | GKAPKL | | S | | SGSGSG | | PRYT | | VDIK |
| | | ASVGDR | | | | LIS | | | | TEFTLT | | | | |
| | | VTITC | | | | | | | | ISSLQP | | | | |
| | | | | | | | | | | EDFATY | | | | |
| | | | | | | | | | | YC | | | | |
| ADI-81845 | IGKV1D-39 | EIELTQ | 3598 | RASQSI | 3791 | WYQQKP | 2275 | ATSRLQ | 4082 | GVPSRF | 2534 | QQSYSM | 4411 | FGQGTK |
| | | SPSPLS | | GKYLN | | GRAPKL | | S | | SGSGSG | | PPWT | | VEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81860 | IGKV1D-39 | DITLTQ | 1900 | RASQSI | 2249 | WYQWKP | 3960 | AASSLQ | 2391 | GVPSRF | 2592 | QQSYIN | 4412 | FGQGTR |
| | | SPSSLS | | GTYLN | | GKAPKF | | S | | SGSGSG | | PQVT | | LEIK |
| | | ASVGDR | | | | LIY | | | | TEFTLT | | | | |
| | | VTITC | | | | | | | | ISSLQP | | | | |
| | | | | | | | | | | EDFATY | | | | |
| | | | | | | | | | | YC | | | | |
| ADI-81590 | IGLV1-44 | SYELTQ | 3599 | SGGSSN | 3792 | WYHQLP | 3961 | NNNQRP | 4061 | GVPDRF | 2615 | AAWDDS | 4413 | FGTGTK |
| | | PLSASG | | IGSYPV | | GTAPKL | | S | | SGSKSG | | LNGYV | | LTVL |
| | | TPGQRV | | S | | LIY | | | | TSASLA | | | | |
| | | TISC | | | | | | | | ISGLQS | | | | |
| | | | | | | | | | | EDEADY | | | | |
| | | | | | | | | | | YC | | | | |
| ADI-81708 | IGKV1D-39 | ETTLTQ | 3600 | RASQSI | 3793 | WYQQKP | 3962 | AASSLQ | 2391 | GVPSRF | 2534 | QQSYSS | 4384 | FGQGTK |
| | | SPSSVS | | HTYLN | | GKARKL | | S | | SGSGSG | | PPWT | | VDIK |
| | | ASVGDT | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81772 | 2949 | CAGTCTGAGCTGACTCAGCCACCCTCAGCGTCTGGGACCCCCG | 4701 | QSELTQPPSASGTPG | 5055 | 7 | 7 |
| | | GGCAGAGGGTCTCCATCTCTTGTTCTGGAACCAACTCCAACAT | | QRVSISCSGTNSNIG | | | |
| | | CGGAGGTAATACTGTAAACTGGTATCAGCAGCTCCCAGGAACG | | GNTVNWYQQLPGTAP | | | |
| | | GCCCCCAAACTGCTCATCTATAATAATTATCAGCGGCCCTCAG | | KLLIYNNYQRPSGVP | | | |
| | | GGGTCCCTGACCGATTCTCTGGCTCCAAGTCTGGCACCTCAGC | | DRFSGSKSGTSASLA | | | |
| | | CTCCCTGGCCATCAGTGGACTCCAGTCTGAGGATGAGGCTGAA | | ISGLQSEDEAEYYCA | | | |
| | | TATTACTGTGCAACATGGGATGACAGCCTGGATGGTCATAATG | | TWDDSLDGHNVFGTG | | | |
| | | TCTTCGGAACTGGGACCAAGCTCACCGTCCTA | | TKLTVL | | | |
| ADI-81834 | 2948 | CAGTTTGTGGTGACGCAGCCGCCCTCAGTGTTTGGGGCCCCAG | 4702 | QFVVTQPPSVFGAPG | 5056 | 7 | 6 |
| | | GGCAGAGGGTCACCATCTCCTGCACTGGGAGCAACTCCAACAT | | QRVTISCTGSNSNIG | | | |
| | | CGGGGCAGGTTATGATGTTCACTGGTATCAGCAGCTTCCAGGA | | AGYDVHWYQQLPGTA | | | |
| | | ACAGCCCCCAAACTCCTCATCTATGACAACAACAATCGGCCCT | | PKLLIYDNNNRPSGV | | | |
| | | CAGGGGTCCCTGACCGATTCTCTGGCTCCAAGTTTGACACCTC | | PDRFSGSKFDTSASL | | | |
| | | AGCCTCCCTGGCCATCACTGGGCTCCAGGCTGAGGATGAGGCT | | AITGLQAEDEADYYC | | | |
| | | GATTATTACTGCCAGTCCTATGACAGCAGCCTGAGTGATTCGG | | QSYDSSLSDSVFGGG | | | |
| | | TGTTCGGCGGAGGGACCCAGCTCACCGTCCTA | | TQLTVL | | | |
| ADI-81757 | 2937 | GAAATCACACTGACCCAGTCTCCTTCCACCCTGTCTGCATCTG | 4703 | EITLTQSPSTLSASV | 5057 | 7 | 7 |
| | | TAGGAGACAGAGTCACCATCACTTGCCGGGCCAGTCAGAGTAT | | GDRVTITCRASQSIS | | | |
| | | TAGTAGGTGGTTGGCCTGGTATCAGCAGAAACCAGGGAAATCC | | RWLAWYQQKPGKSPN | | | |
| | | CCTAACCTCCTGATCTATAAGGCGTCTAGTTTACAAAGTGGGG | | LLIYKASSLQSGVPA | | | |
| | | TCCCAGCAAGGTTCAGCGGCAGTGCATCTGGGACAGAATTCAC | | RFSGSASGTEFTLTI | | | |
| | | TCTCACCATCAGCCGCCTGCAGCCTGACGATTTTGCAACTTAT | | SRLQPDDFATYYCQQ | | | |
| | | TACTGCCAACAGTATGATACTTATCCGTGGACGTTCGGCCAGG | | YDTYPWTFGQGTKVE | | | |
| | | GGACCAAGGTGGAGATCAAA | | IK | | | |
| ADI-81835 | 2944 | GAAATCCCAATGACCCAGTCTCCATCCTTCCTGTCTGCATCTG | 4704 | EIPMTQSPSFLSASV | 5058 | 7 | 6 |
| | | TAGGAGACAGAGTCACCATCACTTGCCGGGCCAGTCAGGGCAT | | GDRVTITCRASQGIP | | | |
| | | TCCCAGTTTTTTAGCCTGGTATCAGCAAAAACCAGGGAAAGCC | | SFLAWYQQKPGKAPK | | | |
| | | CCTAAGCTCCTGATCTCTGGTGTATCCACTTTGCAAAGTGGGG | | LLISGVSTLQSGVPL | | | |
| | | TCCCATTAAGGTTCAGCGGCAGTGGATCTGGGACAGAATTCAC | | RFSGSGSGTEFTLTI | | | |
| | | TCTCACAATCAGCAGCCTGCAGCCTGAAGATTTTGCAACTTAT | | SSLQPEDFATYYCQY | | | |
| | | TACTGTCAATACCTTGATACTTACCCTCGGTACACTTTTGGCC | | LDTYPRYTFGQGTKV | | | |
| | | AGGGGACCAAGGTGGATATCAAA | | DIK | | | |
| ADI-81845 | 2937 | GAAATCGAGTTGACCCAGTCTCCATCCCCCCTGTCTGCATCTG | 4705 | EIELTQSPSPLSASV | 5059 | 7 | 6 |
| | | TAGGAGACAGAGTCACCATCACTTGCCGGGCAAGTCAGAGCAT | | GDRVTITCRASQSIG | | | |
| | | AGGCAAGTATTTAAATTGGTATCAGCAGAAACCAGGGAGAGCC | | KYLNWYQQKPGRAPK | | | |
| | | CCTAAGCTCCTGATTTACGCTACATCCCGTTTGCAAAGTGGGG | | LLIYATSRLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAACCTGAAGATTTTGCAACTTAT | | SSLQPEDFATYYCQQ | | | |
| | | TACTGTCAGCAGAGTTACAGTATGCCTCCGTGGACGTTCGGCC | | SYSMPPWTFGQGTKV | | | |
| | | AAGGGACCAAGGTGGAGATCAAA | | EIK | | | |
| ADI-81860 | 2940 | GACATCACACTGACCCAGTCTCCATCCTCCCTGTCTGCATCTG | 4706 | DITLTQSPSSLSASV | 5060 | 7 | 5 |
| | | TAGGAGACCGAGTCACCATCACTTGCCGGGCAAGTCAGAGCAT | | GDRVTITCRASQSIG | | | |
| | | TGGCACCTATTTAAATTGGTATCAGTGGAAACCAGGGAAAGCC | | TYLNWYQWKPGKAPK | | | |
| | | CCTAAGTTTTTGATCTATGCTGCATCCAGTTTGCAAAGTGGGG | | FLIYAASSLQSGVPS | | | |
| | | TCCCATCAAGGTTCAGTGGCAGTGGATCTGGGACAGAGTTCAC | | RFSGSGSGTEFTLTI | | | |
| | | TCTCACCATCAGCAGTCTGCAACCTGAAGATTTTGCAACTTAC | | SSLQPEDFATYYCQQ | | | |
| | | TACTGTCAACAGAGTTACATTAACCCTCAGGTCACCTTCGGCC | | SYINPQVTFGQGTRL | | | |
| | | AAGGGACACGACTGGAGATTAAA | | EIK | | | |
| ADI-81590 | 2949 | TCGTATGAGCTGACTCAGCCACTCTCAGCGTCTGGGACCCCCG | 4707 | SYELTQPLSASGTPG | 5061 | 7 | 7 |
| | | GGCAGAGGGTCACCATCTCTTGTTCTGGAGGCAGCTCCAACAT | | QRVTISCSGGSSNIG | | | |
| | | CGGAAGTTATCCTGTGAGCTGGTACCACCAGCTCCCAGGAACG | | SYPVSWYHQLPGTAP | | | |
| | | GCCCCCAAACTCCTCATCTATAATAATAATCAGCGGCCCTCAG | | KLLIYNNNQRPSGVP | | | |
| | | GGGTCCCTGACCGATTCTCTGGCTCCAAGTCTGGCACCTCAGC | | DRFSGSKSGTSASLA | | | |
| | | CTCCCTGGCCATCAGTGGGCTCCAGTCTGAGGATGAGGCTGAT | | ISGLQSEDEADYYCA | | | |
| | | TATTACTGTGCAGCATGGGATGACAGCCTGAATGGTTATGTCT | | AWDDSLNGYVFGTGT | | | |
| | | TCGGAACTGGGACCAAGCTCACCGTCCTA | | KLTVL | | | |
| ADI-81708 | 2944 | GAAACAACATTGACCCAGTCTCCATCTTCCGTGTCTGCATCTG | 4708 | ETTLTQSPSSVSASV | 5062 | 7 | 5 |
| | | TAGGAGACACAGTCACCATCACTTGCCGGGCAAGTCAGAGCAT | | GDTVTITCRASQSIH | | | |
| | | TCACACGTATTTAAATTGGTATCAACAGAAACCAGGGAAAGCC | | TYLNWYQQKPGKARK | | | |
| | | CGTAAACTCCTGATATATGCTGCATCCAGTTTGCAAAGTGGGG | | LLIYAASSLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAACCTGAAGATTTTGCAACTTAT | | SSLQPEDFATYYCQQ | | | |
| | | TACTGTCAACAGAGTTACAGTAGTCCTCCGTGGACGTTCGGCC | | SYSSPPWTFGQGTKV | | | |
| | | AAGGGACCAAAGTGGATATCAAA | | DIK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81737 | IGKV3D-15 | EMTITQ | 3601 | RASQSV | 2227 | WYQQKP | 2258 | GASTRA | 2383 | DIPARF | 4234 | QQYNNW | 4414 | FGQGTK |
| | | SPATLF | | SSNLA | | GQAPRL | | T | | SGSGFG | | PRA | | VEIK |
| | | VSPGDR | | | | LIY | | | | AEFTLT | | | | |
| | | ATLSC | | | | | | | | ISSLQF | | | | |
| | | | | | | | | | | EDFAVY | | | | |
| | | | | | | | | | | YC | | | | |
| ADI-81905 | IGKV1-33 | EMTMTQ | 3602 | QAGQDI | 3794 | WYQQEP | 3963 | DASNLE | 2416 | GVPSRF | 4235 | QQYEDL | 4415 | FGGGTK |
| | | SPSSLS | | SNHLS | | GKAPKL | | T | | TGSGSG | | PPT | | VDIK |
| | | ASVGDR | | | | LIY | | | | TDFTFT | | | | |
| | | VTITQ | | | | | | | | ISSLQP | | | | |
| | | | | | | | | | | EDVTTY | | | | |
| | | | | | | | | | | YC | | | | |
| ADI-81618 | IGLV3-25 | SHELTQ | 3603 | SGDALP | 3795 | WYQQKP | 3964 | KDIERP | 4083 | GIPERF | 4236 | QSSDNT | 4416 | FGGGTK |
| | | PPSVSV | | SQYAY | | GQAPLL | | S | | SGSSSG | | GTYVV | | LTVL |
| | | SPGQTA | | | | MIY | | | | TTVTLT | | | | |
| | | RITC | | | | | | | | ISGVQA | | | | |
| | | | | | | | | | | EDEADY | | | | |
| | | | | | | | | | | FC | | | | |
| ADI-81901 | IGKV1D-39 | DIGMTQ | 3604 | RASQTI | 3796 | WYQQRP | 3965 | VASSLQ | 2418 | GVPSRF | 4163 | QQSYST | 4417 | FGQGTK |
| | | SPSSLS | | SNYLN | | GKGPKL | | S | | SGSRSG | | PPT | | VEIK |
| | | AAVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81532 | IGKV1D-39 | EIVLTQ | 1888 | RASQSI | 3797 | WYQHKP | 3966 | AASTLQ | 2392 | GVPSRF | 2536 | QQSYSM | 4411 | FGQGTK |
| | | SPSSLS | | TRYLN | | GNAPKL | | S | | SGSGSG | | PPWT | | VEIK |
| | | ASVGDR | | | | LIY | | | | TDFTLT | | | | |
| | | VTITC | | | | | | | | INSLQP | | | | |
| | | | | | | | | | | EDFATY | | | | |
| | | | | | | | | | | YC | | | | |
| ADI-81537 | IGKV1D-39 | DIQMTQ | 1878 | RASQSI | 3797 | WYQHKP | 3966 | AASTLQ | 2392 | GVPSRF | 2536 | QQSYSM | 4411 | FGQGTK |
| | | SPSSLS | | TRYLN | | GNAPKL | | S | | SGSGSG | | PPWT | | VEIK |
| | | ASVGDR | | | | LIY | | | | TDFTLT | | | | |
| | | VTITC | | | | | | | | INSLQP | | | | |
| | | | | | | | | | | EDFATY | | | | |
| | | | | | | | | | | YC | | | | |
| ADI-81565 | IGLV1-40 | QSVLTQ | 3605 | SGSNSN | 3798 | WYQQLP | 2314 | RNNNRP | 4084 | GVPDRF | 4237 | QSYDSS | 2853 | FGEGTK |
| | | PPSVSG | | IGAGYD | | GTAPKL | | S | | SGSKSA | | LSGVV | | LTVL |
| | | APGQRV | | VH | | LIY | | | | TSASLA | | | | |
| | | TIPC | | | | | | | | ITGLQA | | | | |
| | | | | | | | | | | EDEADY | | | | |
| | | | | | | | | | | YC | | | | |
| ADI-81811 | IGLV3-10 | QYVLTQ | 3606 | SGDALP | 3799 | WYQQKS | 3967 | EDRERP | 4085 | GIPERF | 4238 | YSTDSS | 4418 | FGGGTK |
| | | PPSVSV | | RKYAH | | GQAPVL | | S | | SGSRSG | | GTQGV | | LTVL |
| | | SPGQTA | | | | VIH | | | | TVATLT | | | | |
| | | RITC | | | | | | | | ISGAQV | | | | |
| | | | | | | | | | | EDEADY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81737 | 2937 | GAAATGACAATCACGCAGTCTCCAGCCACCCTGTTTGTGTCTC | 4709 | EMTITQSPATLFVSP | 5063 | 7 | 6 |
| | | CAGGGGATAGAGCCACCCTCTCCTGCAGGGCCAGTCAGAGTGT | | GDRATLSCRASQSVS | | | |
| | | TAGCAGCAACTTAGCCTGGTACCAGCAGAAACCTGGCCAGGCT | | SNLAWYQQKPGQAPR | | | |
| | | CCCAGGCTCCTCATCTATGGTGCATCCACCAGGGCCACTGATA | | LLIYGASTRATDIPA | | | |
| | | TCCCAGCCAGGTTCAGTGGCAGTGGGTTTGGGGCAGAGTTCAC | | RFSGSGFGAEFTLTI | | | |
| | | TCTCACCATCAGCAGCCTGCAGTTTGAAGATTTTGCAGTTTAT | | SSLQFEDFAVYYCQQ | | | |
| | | TACTGTCAGCAGTATAATAACTGGCCTCGGGCGTTCGGCCAAG | | YNNWPRAFGQGTKVE | | | |
| | | GGACCAAGGTGGAAATCAAA | | IK | | | |
| ADI-81905 | 2938 | GAAATGACAATGACGCAGTCTCCATCCTCCCTGTCTGCATCTG | 4710 | EMTMTQSPSSLSASV | 5064 | 7 | 7 |
| | | TAGGAGACAGAGTCACCATCACTTGCCAGGCTGGTCAGGACAT | | GDRVTITCQAGQDIS | | | |
| | | TTCCAACCATTTAAGTTGGTATCAGCAGGAACCAGGGAAAGCC | | NHLSWYQQEPGKAPK | | | |
| | | CCTAAGCTCCTGATCTACGATGCATCCAATTTGGAAACAGGGG | | LLIYDASNLETGVPS | | | |
| | | TCCCATCAAGGTTCACTGGAAGTGGATCTGGGACAGATTTTAC | | RFTGSGSGTDFTFTI | | | |
| | | TTTCACCATCAGCAGCCTGCAGCCTGAAGATGTTACAACATAT | | SSLQPEDVTTYYCQQ | | | |
| | | TACTGTCAACAGTATGAGGATCTCCCTCCGACTTTCGGCGGAG | | YEDLPPTFGGGTKVD | | | |
| | | GGACCAAAGTGGATATCAAA | | IK | | | |
| ADI-81618 | 2939 | TCGCATGAGCTGACACAGCCACCCTCGGTGTCAGTGTCCCCAG | 4711 | SHELTQPPSVSVSPG | 5065 | 7 | 5 |
| | | GACAGACGGCCAGGATCACCTGCTCTGGAGATGCCTTGCCAAG | | QTARITCSGDALPSQ | | | |
| | | CCAATATGCTTATTGGTACCAACAGAAGCCAGGCCAGGCCCCT | | YAYWYQQKPGQAPLL | | | |
| | | CTACTAATGATCTATAAAGACATTGAGAGGCCCTCAGGGATCC | | MIYKDIERPSGIPER | | | |
| | | CTGAGCGATTCTCTGGCTCCAGCTCAGGGACAACAGTCACGTT | | FSGSSSGTTVTLTIS | | | |
| | | GACCATCAGTGGAGTCCAGGCAGAAGACGAGGCTGACTATTTC | | GVQAEDEADYFCQSS | | | |
| | | TGTCAATCATCAGACAATACTGGTACTTATGTGGTGTTCGGCG | | DNTGTYVVFGGGTKL | | | |
| | | GAGGGACCAAGCTGACCGTCCTA | | TVL | | | |
| ADI-81901 | 2937 | GACATCGGGATGACCCAGTCTCCATCCTCCCTGTCTGCAGCTG | 4712 | DIGMTQSPSSLSAAV | 5066 | 7 | 7 |
| | | TAGGAGACAGAGTCACCATCACTTGCCGGGCAAGTCAGACCAT | | GDRVTITCRASQTIS | | | |
| | | TAGCAACTATTTAAATTGGTATCAGCAGAGACCAGGGAAAGGC | | NYLNWYQQRPGKGPK | | | |
| | | CCTAAGCTCCTGATCTATGTTGCATCCAGTTTGCAAAGTGGGG | | LLIYVASSLQSGVPS | | | |
| | | TCCCATCAAGGTTCAGTGGCAGTAGATCTGGGACAGATTTCAC | | RFSGSRSGTDFTLTI | | | |
| | | TCTCACCATCAGCAGTCTGCAACCTGAAGATTTTGCAACTTAC | | SSLQPEDFATYYCQQ | | | |
| | | TACTGTCAACAGAGTTATAGTACCCCTCCCACTTTTGGCCAGG | | SYSTPPTFGQGTKVE | | | |
| | | GGACCAAGGTGGAGATCAAA | | IK | | | |
| ADI-81532 | 2937 | GAAATTGTATTGACACAGTCTCCATCCTCCCTGTCTGCATCTG | 4713 | EIVLTQSPSSLSASV | 5067 | 7 | 6 |
| | | TAGGAGACAGAGTCACCATCACTTGCCGGGCAAGTCAGAGCAT | | GDRVTITCRASQSIT | | | |
| | | TACCCGGTATTTAAATTGGTATCAACACAAACCAGGGAACGCC | | RYLNWYQHKPGNAPK | | | |
| | | CCTAAGCTCCTGATCTATGCTGCATCCACTTTGCAAAGTGGGG | | LLIYAASTLQSGVPS | | | |
| | | TCCCATCAAGGTTCAGTGGCAGTGGATCTGGTACAGATTTCAC | | RFSGSGSGTDFTLTI | | | |
| | | TCTCACCATCAACAGTCTGCAACCTGAAGATTTTGCAACTTAC | | NSLQPEDFATYYCQQ | | | |
| | | TACTGTCAACAGAGTTACAGTATGCCTCCGTGGACGTTCGGCC | | SYSMPPWTFGQGTKV | | | |
| | | AAGGGACCAAGGTGGAAATCAAA | | EIK | | | |
| ADI-81537 | 2937 | GACATCCAGATGACCCAGTCTCCATCCTCCCTGTCTGCATCTG | 4714 | DIQMTQSPSSLSASV | 5068 | 7 | 6 |
| | | TAGGAGACAGAGTCACCATCACTTGCCGGGCAAGTCAGAGCAT | | GDRVTITCRASQSIT | | | |
| | | TACCCGGTATTTAAATTGGTATCAACACAAACCAGGGAACGCC | | RYLNWYQHKPGNAPK | | | |
| | | CCTAAGCTCCTGATCTATGCTGCATCCACTTTGCAAAGTGGGG | | LLIYAASTLQSGVPS | | | |
| | | TCCCATCAAGGTTCAGTGGCAGTGGATCTGGTACAGATTTCAC | | RFSGSGSGTDFTLTI | | | |
| | | TCTCACCATCAACAGTCTGCAACCTGAAGATTTTGCAACTTAC | | NSLQPEDFATYYCQQ | | | |
| | | TACTGTCAACAGAGTTACAGTATGCCTCCGTGGACGTTCGGCC | | SYSMPPWTFGQGTKV | | | |
| | | AAGGGACCAAGGTGGAAATCAAA | | EIK | | | |
| ADI-81565 | 4598 | CAGTCTGTCTTGACGCAGCCGCCCTCAGTGTCTGGGGCCCCAG | 4715 | QSVLTQPPSVSGAPG | 5069 | 7 | 6 |
| | | GGCAGAGGGTCACCATCCCCTGCAGTGGGAGCAACTCCAACAT | | QRVTIPCSGSNSNIG | | | |
| | | CGGGGCAGGTTATGATGTACACTGGTACCAGCAACTTCCAGGA | | AGYDVHWYQQLPGTA | | | |
| | | ACAGCCCCCAAACTCCTCATTTATAGAAACAACAATCGGCCCT | | PKLLIYRNNNRPSGV | | | |
| | | CAGGGGTCCCTGACCGCTTCTCTGGCTCCAAGTCTGCCACCTC | | PDRFSGSKSATSASL | | | |
| | | AGCCTCCCTGGCCATCACTGGGCTCCAGGCTGAGGATGAGGCT | | AITGLQAEDEADYYC | | | |
| | | GATTATTACTGCCAGTCGTATGACAGCAGCCTGAGTGGTGTGG | | QSYDSSLSGVVFGEG | | | |
| | | TTTTCGGCGAAGGGACCAAGCTGACCGTCCTA | | TKLTVL | | | |
| ADI-81811 | 2939 | CAGTATGTGCTGACGCAGCCACCCTCGGTGTCAGTGTCCCCAG | 4716 | QYVLTQPPSVSVSPG | 5070 | 7 | 7 |
| | | GACAAACGGCCAGGATCACCTGCTCTGGAGATGCACTGCCAAG | | QTARITCSGDALPRK | | | |
| | | AAAATATGCTCATTGGTACCAGCAGAAGTCAGGCCAGGCCCCT | | YAHWYQQKSGQAPVL | | | |
| | | GTGTTGGTCATCCATGAAGACAGGGAACGACCCTCCGGAATCC | | VIHEDRERPSGIPER | | | |
| | | CTGAGAGATTCTCTGGCTCCAGATCAGGGACAGTGGCCACCTT | | FSGSRSGTVATLTIS | | | |
| | | GACTATCAGTGGGGCCCAGGTGGAGGATGAGGCTGACTACTAC | | GAQVEDEADYYCYST | | | |
| | | TGTTACTCAACAGACAGCAGTGGTACTCAAGGGGTCTTCGGCG | | DSSGTQGVFGGGTKL | | | |
| | | GAGGGACCAAGCTCACCGTCCTA | | TVL | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81883 | IGKV1D-39 | DIRMTQ | 360 | RASQTI | 3796 | WYQQRP | 3965 | VASSLQ | 2418 | GVPSRF | 4163 | QQSYST | 4417 | FGQGTK |
| | | SPSSLS | | SNYLN | | GKGPKL | | S | | SGSRSG | | PPT | | VDIK |
| | | AAVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81686 | IGKV1D-39 | EMTLTQ | 3608 | RASQII | 3800 | WYQQKP | 2263 | AASNLQ | 2408 | GVPSRF | 2534 | QQSYTT | 2833 | FGQGTK |
| | | SPSSLS | | TAYLN | | GKAPNL | | S | | SGSGSG | | PYT | | VEIK |
| | | AYVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81825 | IGKV1D-39 | EITLTQ | 3567 | RASQSI | 2066 | WYQQKP | 3968 | AVSSLK | 4086 | GVPSRF | 4239 | QQSYTT | 4419 | FGGGTK |
| | | SPSSLS | | STYLN | | GKAPQL | | S | | SGSGSG | | PALT | | VDIK |
| | | ASVGDR | | | | LIY | | | | TDFTLT | | | | |
| | | VTITC | | | | | | | | ISSLQP | | | | |
| | | | | | | | | | | EDSAAY | | | | |
| | | | | | | | | | | YC | | | | |
| ADI-81748 | IGKV1-12 | EIPVTQ | 3609 | RASQHF | 3801 | WYQLKP | 3909 | AASTLQ | 2392 | GVPSRF | 4240 | QQANSF | 4420 | FGQGTK |
| | | SPSSVS | | SNWLA | | GKAPKL | | S | | SGSGSG | | PYT | | VEIK |
| | | ASVGDR | | | | LIY | | | | TDFTLT | | | | |
| | | VTITC | | | | | | | | ISSLLP | | | | |
| | | | | | | | | | | EDFATY | | | | |
| | | | | | | | | | | YC | | | | |
| ADI-81840 | IGKV1-5 | DIRMTQ | 3610 | RASQSI | 3802 | WSQQKP | 3969 | GASTLG | 4087 | GVPSRF | 4241 | QQYKSY | 4421 | FGQGTK |
| | | SPSTLS | | SGWLA | | GKAPKL | | S | | SGSGFG | | SYT | | VEIK |
| | | ASVGDR | | | | LIY | | | | TEFTLT | | | | |
| | | VTITC | | | | | | | | ISSLQP | | | | |
| | | | | | | | | | | DDFATY | | | | |
| | | | | | | | | | | YC | | | | |
| ADI-81692 | IGLV2-14 | QFVLNQ | 3611 | TGTSSD | 3803 | WYQHHP | 3970 | EVTNRP | 2465 | GDSDRF | 4242 | SSYTSS | 4422 | FGTGTK |
| | | PASVSG | | VGDYNY | | GTAPKL | | S | | SGSKSG | | NTLV | | LTVL |
| | | SPGQSI | | VS | | MIY | | | | NTASLT | | | | |
| | | TISC | | | | | | | | ISGLQA | | | | |
| | | | | | | | | | | EDEADY | | | | |
| | | | | | | | | | | YC | | | | |
| ADI-81730 | IGKV3-20 | EIVMTQ | 3612 | RASQSI | 3804 | WYQQKP | 2258 | ATSTRA | 4088 | GIPDRF | 2616 | QQYGSS | 4423 | FGQGTK |
| | | SPGSLS | | SRTYLA | | GQAPRL | | T | | SGSGSG | | PMYT | | VDIK |
| | | LSPGER | | | | LIY | | | | TDFTLT | | | | |
| | | ATLSC | | | | | | | | ISRLEP | | | | |
| | | | | | | | | | | EDFAVY | | | | |
| | | | | | | | | | | YC | | | | |
| ADI-81889 | IGKV1-5 | EMPMTQ | 3613 | RASQSI | 3805 | WYQQKP | 3971 | KASSLE | 2386 | GVPSRF | 4243 | QQYNSY | 4424 | FGGGTK |
| | | SPSTLS | | GSRLA | | GKAPSL | | S | | SGSGSG | | SLT | | VEIK |
| | | ASVGDR | | | | LIY | | | | TEFTLT | | | | |
| | | VTITC | | | | | | | | ISSLQP | | | | |
| | | | | | | | | | | DDLATY | | | | |
| | | | | | | | | | | FC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81883 | 2944 | GACATCCGGATGACCCAGTCTCCATCCTCCCTGTCTGCAGCTG | 4717 | DIRMTQSPSSLSAAV | 5071 | 7 | 7 |
| | | TAGGAGACAGAGTCACCATCACTTGCCGGGCAAGTCAGACCAT | | GDRVTITCRASQTIS | | | |
| | | TAGCAACTATTTAAATTGGTATCAGCAGAGACCAGGGAAAGGC | | NYLNWYQQRPGKGPK | | | |
| | | CCTAAGCTCCTGATCTATGTTGCATCCAGTTTGCAAAGTGGGG | | LLIYVASSLQSGVPS | | | |
| | | TCCCATCAAGGTTCAGTGGCAGTAGATCTGGGACAGATTTCAC | | RFSGSRSGTDFTLTI | | | |
| | | TCTCACCATCAGCAGTCTGCAACCTGAAGATTTTGCAACTTAC | | SSLQPEDFATYYCQQ | | | |
| | | TACTGTCAACAGAGTTATAGTACCCCTCCCACTTTTGGCCAGG | | SYSTPPTFGQGTKVD | | | |
| | | GGACCAAAGTGGATATCAAA | | IK | | | |
| ADI-81686 | 2937 | GAAATGACACTGACGCAGTCTCCATCCTCCCTGTCTGCATATG | 4718 | EMTLTQSPSSLSAYV | 5072 | 7 | 6 |
| | | TAGGAGACAGAGTCACCATCACTTGCCGGGCAAGTCAGATCAT | | GDRVTITCRASQIIT | | | |
| | | TACCGCCTATTTAAATTGGTATCAGCAGAAACCAGGTAAAGCC | | AYLNWYQQKPGKAPN | | | |
| | | CCTAACCTCCTGATCTATGCTGCCTCCAATTTGCAAAGTGGGG | | LLIYAASNLQSGVPS | | | |
| | | TCCCATCAAGGTTCAGTGGCAGTGGATCTGGGACAGATTTCAC | | RFSGSGSGTDFTLTI | | | |
| | | TCTCACCATTAGCAGTCTGCAACCTGAAGATTTTGCAACTTAC | | SSLQPEDFATYYCQQ | | | |
| | | TACTGTCAACAGAGTTACACTACCCCGTACACCTTTGGCCAGG | | SYTTPYTFGQGTKVE | | | |
| | | GGACCAAGGTGGAGATCAAA | | IK | | | |
| ADI-81825 | 2938 | GAAATCACACTCACCCAGTCTCCATCCTCCCTGTCTGCATCTG | 4719 | EITLTQSPSSLSASV | 5073 | 7 | 6 |
| | | TTGGAGACAGAGTCACCATCACTTGCCGGGCAAGTCAGAGCAT | | GDRVTITCRASQSIS | | | |
| | | TAGCACCTATTTAAATTGGTATCAGCAGAAACCAGGGAAAGCC | | TYLNWYQQKPGKAPQ | | | |
| | | CCTCAGCTCCTGATCTATGCTGTTTCCAGTTTGAAAAGTGGGG | | LLIYAVSSLKSGVPS | | | |
| | | TCCCATCAAGGTTCAGTGGCAGTGGGTCTGGGACAGACTTCAC | | RFSGSGSGTDFTLTI | | | |
| | | TCTCACCATCAGCAGTCTGCAACCTGAGGATTCTGCAGCTTAC | | SSLQPEDSAAYYCQQ | | | |
| | | TATTGTCAACAGAGTTACACTACCCCCGCGCTCACTTTCGGCG | | SYTTPALTFGGGTKV | | | |
| | | GAGGGACCAAGGTGGATATCAAA | | DIK | | | |
| ADI-81748 | 2937 | GAAATCCCGGTGACCCAGTCTCCATCTTCTGTGTCTGCATCTG | 4720 | EIPVTQSPSSVSASV | 5074 | 7 | 6 |
| | | TAGGAGACAGAGTCACCATCACTTGTCGGGCGAGTCAACATTT | | GDRVTITCRASQHFS | | | |
| | | TAGCAACTGGTTAGCCTGGTATCAGCTGAAACCAGGGAAAGCC | | NWLAWYQLKPGKAPK | | | |
| | | CCTAAACTCCTGATCTATGCTGCATCCACTTTGCAGAGTGGGG | | LLIYAASTLQSGVPS | | | |
| | | TCCCATCAAGGTTCAGCGGCAGTGGGTCTGGGACAGATTTCAC | | RFSGSGSGTDFTLTI | | | |
| | | TCTCACTATCAGCAGCCTGCTGCCTGAAGATTTTGCAACTTAC | | SSLLPEDFATYYCQQ | | | |
| | | TATTGTCAACAGGCTAACAGTTTCCCGTACACTTTTGGCCAGG | | ANSFPYTFGQGTKVE | | | |
| | | GGACCAAGGTGGAAATCAAA | | IK | | | |
| ADI-81840 | 2937 | GACATCCGGATGACCCAGTCTCCTTCCACCCTGTCTGCATCTG | 4721 | DIRMTQSPSTLSASV | 5075 | 7 | 6 |
| | | TAGGAGACAGAGTCACCATCACTTGCCGGGCCAGTCAGAGTAT | | GDRVTITCRASQSIS | | | |
| | | TAGTGGCTGGTTGGCCTGGTCTCAGCAGAAACCAGGGAAAGCC | | GWLAWSQQKPGKAPK | | | |
| | | CCTAAGCTCCTGATCTATGGGGCATCTACTTTAGGAAGTGGGG | | LLIYGASTLGSGVPS | | | |
| | | TCCCATCAAGGTTCAGCGGCAGTGGATTTGGGACAGAATTCAC | | RFSGSGFGTEFTLTI | | | |
| | | TCTCACCATCAGCAGCCTGCAGCCTGATGATTTTGCAACTTAT | | SSLQPDDFATYYCQQ | | | |
| | | TACTGCCAACAATATAAAAGTTATTCCTATACTTTTGGCCAGG | | YKSYSYTFGQGTKVE | | | |
| | | GGACCAAGGTGGAAATCAAA | | IK | | | |
| ADI-81692 | 2949 | CAGTTTGTTCTGAATCAGCCTGCCTCCGTGTCTGGGTCTCCTG | 4722 | QFVLNQPASVSGSPG | 5076 | 7 | 6 |
| | | GACAGTCGATCACCATCTCCTGCACTGGAACCAGCAGTGACGT | | QSITISCTGTSSDVG | | | |
| | | TGGTGATTATAACTATGTCTCCTGGTACCAACACCACCCAGGC | | DYNYVSWYQHHPGTA | | | |
| | | ACCGCCCCCAAACTCATGATTTATGAGGTCACTAATCGGCCCT | | PKLMIYEVTNRPSGD | | | |
| | | CAGGGGATTCTGATCGCTTCTCTGGCTCCAAGTCTGGCAACAC | | SDRFSGSKSGNTASL | | | |
| | | GGCCTCCCTGACCATCTCTGGCCTCCAGGCTGAGGACGAGGCT | | TISGLQAEDEADYYC | | | |
| | | GATTATTACTGCAGCTCATATACAAGCAGCAACACTCTCGTCT | | SSYTSSNTLVFGTGT | | | |
| | | TCGGAACTGGGACCAAGCTCACCGTCCTA | | KLTVL | | | |
| ADI-81730 | 2944 | GAAATTGTGATGACGCAGTCTCCAGGCTCCCTGTCTTTGTCTC | 4723 | EIVMTQSPGSLSLSP | 5077 | 7 | 7 |
| | | CAGGGGAAAGAGCCACCCTCTCCTGCAGGGCCAGTCAGAGTAT | | GERATLSCRASQSIS | | | |
| | | TAGCAGAACCTACTTAGCCTGGTACCAGCAGAAACCTGGCCAG | | RTYLAWYQQKPGQAP | | | |
| | | GCTCCCAGGCTCCTCATCTATGCTACATCCACCAGGGCCACTG | | RLLIYATSTRATGIP | | | |
| | | GCATCCCAGACAGGTTCAGTGGCAGTGGGTCTGGGACAGACTT | | DRFSGSGSGTDFTLT | | | |
| | | CACTCTCACCATCAGCAGACTGGAGCCTGAAGATTTTGCAGTG | | ISRLEPEDFAVYYCQ | | | |
| | | TATTACTGTCAGCAGTATGGTAGTTCACCTATGTATACTTTTG | | QYGSSPMYTFGQGTK | | | |
| | | GCCAGGGGACCAAGGTGGATATCAAA | | VDIK | | | |
| ADI-81889 | 2936 | GAAATGCCAATGACGCAGTCTCCTTCCACCCTGTCTGCATCTG | 4724 | EMPMTQSPSTLSASV | 5078 | 6 | 5 |
| | | TGGGAGACAGAGTCACCATCACTTGCCGGGCCAGTCAGAGTAT | | GDRVTITCRASQSIG | | | |
| | | TGGTAGCAGGTTGGCCTGGTATCAGCAGAAACCAGGGAAAGCC | | SRLAWYQQKPGKAPS | | | |
| | | CCTAGCCTCTTGATCTATAAGGCATCTAGTTTAGAAAGTGGGG | | LLIYKASSLESGVPS | | | |
| | | TCCCATCAAGGTTCAGCGGCAGTGGATCTGGGACAGAATTCAC | | RFSGSGSGTEFTLTI | | | |
| | | TCTCACCATCAGCAGCCTGCAGCCTGATGATTTGGCAACTTAT | | SSLQPDDLATYFCQQ | | | |
| | | TTCTGCCAACAGTATAATAGTTACTCCCTCACTTTCGGCGGAG | | YNSYSLTFGGGTKVE | | | |
| | | GGACCAAGGTGGAAATCAAA | | IK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81608 | IGKV1-33 | DIQLTQ | 1876 | QASQDI | 2212 | WYQQKP | 2257 | DASNYD | 4089 | GVPSRF | 4244 | QQYDSL | 4425 | FGGGTK |
| | | SPSSLS | | SNYLN | | GKAPKL | | T | | SGTGSG | | PLT | | VEIK |
| | | ASVGDR | | | | LIY | | | | TDFTFT | | | | |
| | | VTITC | | | | | | | | ISSLQP | | | | |
| | | | | | | | | | | EDIAIY | | | | |
| | | | | | | | | | | FC | | | | |
| ADI-81616 | IGKV4-1 | DIQLTQ | 3614 | KSSQSV | 3806 | WYQQRP | 2381 | WASTRE | 2422 | GVPDRF | 2619 | QQYYSW | 4426 | FGQGTK |
| | | SPDSLA | | FSSSNN | | GQPPKL | | S | | SGSGSG | | YS | | VEIK |
| | | VPLGER | | RNNLA | | LIY | | | | TDFTLT | | | | |
| | | ATINC | | | | | | | | ISSLQA | | | | |
| | | | | | | | | | | EDVAVY | | | | |
| | | | | | | | | | | YC | | | | |
| ADI-81734 | IGKV1D-39 | DITMTQ | 1882 | RASQNI | 3807 | WYQQKP | 3972 | AASTLQ | 2392 | GVPSRF | 2534 | QQSYES | 4427 | FGQGTK |
| | | SPSSLS | | NGYLN | | GKGPKV | | S | | SGSGSG | | PPWT | | VDIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81767 | IGKV3-20 | EMTLTQ | 1963 | RASQSV | 3808 | WYQQKP | 2258 | AASGRA | 4090 | GIPDRF | 2616 | QQYGSS | 2929 | FGQGTK |
| | | SPGTLS | | GSGFLA | | GQAPRL | | P | | SGSGSG | | PYT | | VDIK |
| | | LSPGER | | | | LIY | | | | TDFTLT | | | | |
| | | ATLSC | | | | | | | | ISRLEP | | | | |
| | | | | | | | | | | EDFAVY | | | | |
| | | | | | | | | | | YC | | | | |
| ADI-81688 | IGLV1-47 | NYVLTQ | 3615 | SGNRSN | 3809 | WYQQFP | 2324 | KNNQRP | 4091 | GVPDRF | 2669 | ASWHNG | 4428 | FGGGTK |
| | | PPSASG | | IGDNYV | | GTAPKL | | S | | SGSKSG | | LSGSNW | | LTVL |
| | | TPGQRV | | Y | | LIY | | | | TSASLA | | A | | |
| | | TISC | | | | | | | | ISGLRS | | | | |
| | | | | | | | | | | EDEADY | | | | |
| | | | | | | | | | | YC | | | | |
| ADI-81697 | IGKV1D-39 | DITLTQ | 1900 | RASQNI | 3771 | WYQHRP | 3973 | AASSLH | 2426 | GVPSRF | 2534 | QQSYSI | 4429 | FGQGTK |
| | | SPSSLS | | NSYLN | | GIAPKL | | S | | SGSGSG | | SPYT | | VEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81549 | IGLV3-21 | QHVLTQ | 3616 | GGNNIG | 2215 | WYQQRP | 3974 | YDSDRP | 2498 | GIPERF | 4245 | QVWDSR | 4430 | FGTGTK |
| | | PPSLSV | | TKSVH | | GQAPVM | | S | | SGSNSG | | GVYV | | VTVL |
| | | APGRTA | | | | VIY | | | | NTATLT | | | | |
| | | RITC | | | | | | | | ISRAEA | | | | |
| | | | | | | | | | | GDEADY | | | | |
| | | | | | | | | | | YC | | | | |
| ADI-81562 | IGKV1-9 | DIQLTQ | 3617 | RASQGF | 3810 | WYQQKP | 3975 | AASPLQ | 4092 | GVPSRF | 4246 | QQLNSY | 4431 | FGGGTK |
| | | SPSFLS | | GSYVA | | GKAPKL | | S | | SGSGSG | | PIT | | VEIK |
| | | ASVGDR | | | | LTY | | | | TEFTLT | | | | |
| | | VTITC | | | | | | | | ISSLQP | | | | |
| | | | | | | | | | | EDFATY | | | | |
| | | | | | | | | | | FC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81608 | 2936 | GACATCCAGTTGACCCAGTCTCCATCCTCCCTGTCTGCATCTG | 4725 | DIQLTQSPSSLSASV | 5079 | 6 | 5 |
| | | TGGGAGACAGAGTCACCATCACTTGCCAGGCGAGTCAGGACAT | | GDRVTITCQASQDIS | | | |
| | | AAGCAACTATTTAAATTGGTATCAGCAAAAACCAGGGAAAGCC | | NYLNWYQQKPGKAPK | | | |
| | | CCAAAGCTCCTGATCTACGATGCATCCAATTACGACACAGGGG | | LLIYDASNYDTGVPS | | | |
| | | TCCCATCAAGGTTCAGTGGGACTGGATCTGGGACAGATTTTAC | | RFSGTGSGTDFTFTI | | | |
| | | TTTCACCATCAGCAGCCTGCAGCCTGAAGATATTGCAATATAT | | SSLQPEDIAIYFCQQ | | | |
| | | TTCTGTCAACAGTATGATAGTCTCCCCCTCACTTTCGGCGGAG | | YDSLPLTFGGGTKVE | | | |
| | | GGACCAAGGTGGAGATCAAA | | IK | | | |
| ADI-81616 | 2937 | GACATCCAGTTGACCCAGTCTCCAGACTCCCTGGCTGTGCCTC | 4726 | DIQLTQSPDSLAVPL | 5080 | 6 | 6 |
| | | TGGGCGAGAGGGCCACCATCAACTGCAAGTCCAGCCAGAGTGT | | GERATINCKSSQSVF | | | |
| | | TTTCTCCAGCTCCAACAATAGGAACAACTTAGCTTGGTACCAG | | SSSNNRNNLAWYQQR | | | |
| | | CAGAGACCAGGACAGCCTCCTAAGTTGCTCATTTACTGGGCAT | | PGQPPKLLIYWASTR | | | |
| | | CTACCCGGGAATCCGGGGTCCCTGACCGCTTCAGTGGCAGCGG | | ESGVPDRFSGSGSGT | | | |
| | | GTCTGGGACAGATTTCACTCTCACCATCAGCAGCCTGCAGGCT | | DFTLTISSLQAEDVA | | | |
| | | GAAGATGTGGCAGTTTATTACTGTCAGCAATATTATAGTTGGT | | VYYCQQYYSWYSFGQ | | | |
| | | ACAGTTTTGGCCAGGGGACCAAGGTGGAGATCAAA | | GTKVEIK | | | |
| ADI-81734 | 2944 | GACATCACAATGACCCAGTCTCCATCCTCCCTGTCTGCATCTG | 4727 | DITMTQSPSSLSASV | 5081 | 6 | 6 |
| | | TAGGAGACAGAGTCACCATCACTTGCCGGGCAAGTCAGAACAT | | GDRVTITCRASQNIN | | | |
| | | TAACGGCTATTTAAATTGGTATCAGCAAAAACCAGGGAAAGGC | | GYLNWYQQKPGKGPK | | | |
| | | CCTAAGGTCCTGATCTACGCTGCATCCACTTTGCAAAGTGGAG | | VLIYAASTLQSGVPS | | | |
| | | TCCCATCAAGGTTCAGTGGCAGTGGTTCTGGGACAGATTTCAC | | RFSGSGSGTDFTLTI | | | |
| | | TCTCACCATCAGCAGTCTGCAACCTGAAGATTTTGCAACTTAC | | SSLQPEDFATYYCQQ | | | |
| | | TACTGTCAACAGAGTTATGAATCCCCCCCGTGGACGTTCGGCC | | SYESPPWTFGQGTKV | | | |
| | | AAGGGACCAAGGTGGATATCAAA | | DIK | | | |
| ADI-81767 | 2944 | GAAATGACACTGACCCAGTCTCCAGGCACCCTGTCTTTGTCTC | 4728 | EMTLTQSPGTLSLSP | 5082 | 6 | 6 |
| | | CAGGGGAAAGAGCCACCCTCTCCTGCAGGGCCAGTCAGAGTGT | | GERATLSCRASQSVG | | | |
| | | TGGCAGCGGCTTCTTAGCCTGGTACCAGCAGAAACCTGGCCAG | | SGFLAWYQQKPGQAP | | | |
| | | GCTCCCAGGCTCCTCATCTATGCTGCGTCCGGCAGGGCCCCTG | | RLLIYAASGRAPGIP | | | |
| | | GCATCCCAGACAGGTTCAGTGGCAGTGGCTCTGGGACAGACTT | | DRFSGSGSGTDFTLT | | | |
| | | CACTCTCACCATCAGCAGACTGGAGCCTGAAGATTTTGCAGTG | | ISRLEPEDFAVYYCQ | | | |
| | | TATTACTGTCAGCAGTATGGTAGCTCACCGTACACTTTTGGCC | | QYGSSPYTFGQGTKV | | | |
| | | AGGGGACCAAAGTGGATATCAAA | | DIK | | | |
| ADI-81688 | 2939 | AATTATGTGCTGACTCAGCCACCCTCAGCGTCTGGGACCCCCG | 4729 | NYVLTQPPSASGTPG | 5083 | 6 | 5 |
| | | GGCAGAGGGTCACCATCTCTTGCTCTGGAAACAGATCCAACAT | | QRVTISCSGNRSNIG | | | |
| | | AGGAGATAATTATGTATATTGGTACCAGCAGTTCCCAGGAACG | | DNYVYWYQQFPGTAP | | | |
| | | GCCCCCAAACTCCTCATCTATAAGAATAATCAGCGGCCCTCAG | | KLLIYKNNQRPSGVP | | | |
| | | GGGTCCCTGACCGATTCTCTGGCTCCAAGTCTGGCACCTCAGC | | DRFSGSKSGTSASLA | | | |
| | | CTCCCTGGCCATCAGTGGGCTCCGGTCCGAGGATGAGGCTGAT | | ISGLRSEDEADYYCA | | | |
| | | TATTACTGTGCATCATGGCATAACGGCCTGAGTGGCTCTAATT | | SWHNGLSGSNWAFGG | | | |
| | | GGGCGTTCGGCGGAGGGACCAAGCTCACCGTCCTA | | GTKLTVL | | | |
| ADI-81697 | 2937 | GACATCACACTCACGCAGTCTCCATCCTCCCTGTCTGCATCTG | 4730 | DITLTQSPSSLSASV | 5084 | 6 | 6 |
| | | TAGGAGACAGAGTCACCATCACTTGCCGGGCAAGTCAGAACAT | | GDRVTITCRASQNIN | | | |
| | | TAACAGCTATTTAAATTGGTATCAGCACAGACCAGGGATAGCC | | SYLNWYQHRPGIAPK | | | |
| | | CCTAAGCTCCTGATCTATGCTGCATCCAGTTTGCATAGTGGGG | | LLIYAASSLHSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAACCTGAAGATTTTGCAACTTAC | | SSLQPEDFATYYCQQ | | | |
| | | TACTGTCAACAGAGTTACAGTATCTCTCCGTACACTTTTGGCC | | SYSISPYTFGQGTKV | | | |
| | | AGGGGACCAAGGTGGAAATCAAA | | EIK | | | |
| ADI-81549 | 2945 | CAGCATGTGCTGACTCAGCCACCCTCTTTGTCAGTGGCCCCAG | 4731 | QHVLTQPPSLSVAPG | 5085 | 6 | 6 |
| | | GAAGGACGGCCAGGATTACCTGTGGGGGGAACAACATTGGAAC | | RTARITCGGNNIGTK | | | |
| | | TAAGAGTGTGCACTGGTACCAACAGAGGCCAGGCCAGGCCCCT | | SVHWYQQRPGQAPVM | | | |
| | | GTGATGGTCATCTATTATGATAGTGACCGGCCCTCAGGGATCC | | VIYYDSDRPSGIPER | | | |
| | | CTGAGCGATTCTCTGGCTCCAACTCTGGGAACACGGCCACCCT | | FSGSNSGNTATLTIS | | | |
| | | GACCATCAGCAGGGCCGAAGCCGGGGATGAGGCCGACTATTAC | | RAEAGDEADYYCQVW | | | |
| | | TGTCAGGTGTGGGATAGTCGTGGTGTTTATGTCTTCGGAACTG | | DSRGVYVFGTGTKVT | | | |
| | | GGACCAAGGTCACCGTCCTA | | VL | | | |
| ADI-81562 | 2936 | GACATCCAGTTGACCCAGTCTCCATCCTTTCTGTCTGCATCTG | 4732 | DIQLTQSPSFLSASV | 5086 | 6 | 6 |
| | | TAGGAGACAGAGTCACCATCACTTGCCGGGCCAGTCAGGGCTT | | GDRVTITCRASQGFG | | | |
| | | TGGCAGTTATGTAGCCTGGTATCAGCAAAAACCAGGGAAAGCC | | SYVAWYQQKPGKAPK | | | |
| | | CCTAAGCTCCTGACCTATGCTGCATCCCCTTTGCAAAGTGGGG | | LLTYAASPLQSGVPS | | | |
| | | TCCCATCAAGATTCAGCGGCAGTGGATCTGGGACAGAATTCAC | | RFSGSGSGTEFTLTI | | | |
| | | TCTCACAATCAGCAGCCTGCAGCCTGAAGATTTTGCAACTTAT | | SSLQPEDFATYFCQQ | | | |
| | | TTCTGTCAACAACTTAATAGTTACCCTATCACTTTCGGCGGAG | | LNSYPITFGGGTKVE | | | |
| | | GGACCAAGGTGGAGATCAAA | | IK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81813 | IGKV1-6 | DIRVTQ | 3618 | RASQGI | 3811 | WYQQHP | 3976 | AASSLE | 4052 | GVPSRF | 2539 | LQDYSN | 4432 | FGQGTK |
| | | SPSSLS | | RNDLG | | GKAPKL | | T | | SGSGSG | | FWT | | VEIK |
| | | ASVGDR | | | | LIS | | | | TDFTLT | | | | |
| | | VTITC | | | | | | | | ITSLQP | | | | |
| | | | | | | | | | | EDFATY | | | | |
| | | | | | | | | | | YC | | | | |
| ADI-81890 | IGKV1-5 | EMTMTQ | 3619 | RASQSI | 3812 | WYQQKP | 2257 | MTSRLE | 4093 | GVPSRF | 2532 | QHYNSY | 4433 | FGQGTK |
| | | SPSTLS | | SNWLA | | GKAPKL | | G | | SGSGSG | | SEMYT | | VEIK |
| | | ASVGDR | | | | LIY | | | | TEFTLT | | | | |
| | | VTITC | | | | | | | | ISSLQP | | | | |
| | | | | | | | | | | DDFATY | | | | |
| | | | | | | | | | | YC | | | | |
| ADI-81612 | IGLV1-40 | QPVVTQ | 3620 | TGTNSN | 2171 | WYQLLP | 3977 | RNTNRP | 4054 | GVPDRF | 2599 | QSYDSS | 2908 | FGGGTK |
| | | PPSVSG | | IGAGYD | | GTPPKL | | S | | SGSKSG | | LSGSV | | LTVL |
| | | APGQRV | | VH | | LIY | | | | TSASLA | | | | |
| | | TISC | | | | | | | | ITGLQA | | | | |
| | | | | | | | | | | EDEADY | | | | |
| | | | | | | | | | | YC | | | | |
| ADI-81723 | IGKV4-1 | ETTLTQ | 3621 | KSSQSV | 3813 | WYQHKP | 3978 | WASTRQ | 4094 | GVPDRF | 2581 | QQYYSS | 4434 | FGQGTK |
| | | SPDSLA | | LSSSNN | | GQPPKL | | S | | SGSGSG | | PYT | | VDIK |
| | | VSLGER | | RNYLS | | LIY | | | | TDFTLT | | | | |
| | | ATINC | | | | | | | | ISSLQP | | | | |
| | | | | | | | | | | EDVAVY | | | | |
| | | | | | | | | | | YC | | | | |
| ADI-81543 | IGLV1-40 | QSVLTQ | 1948 | TGSNSN | 2143 | WYQQLP | 3979 | DNANRP | 4095 | GVPDRF | 2622 | QSYDSS | 2864 | FGTGTK |
| | | PPSVSG | | IGAGYD | | GSAPKL | | S | | SGSKSD | | LIGSV | | LTVL |
| | | APGQRV | | VH | | LIY | | | | TSASLA | | | | |
| | | TISC | | | | | | | | ITGLQA | | | | |
| | | | | | | | | | | EDEADY | | | | |
| | | | | | | | | | | YC | | | | |
| ADI-81583 | IGKV1D-39 | DIVMTQ | 1902 | RASQSV | 3814 | WYQHKP | 3924 | GASSLQ | 2403 | GVPSRF | 2539 | QQSYST | 4435 | FGGGTK |
| | | SPSSLS | | RTYLN | | GKAPKL | | S | | SGSGSG | | PPLT | | VEIK |
| | | ASVGDR | | | | LIY | | | | TDFTLT | | | | |
| | | VTITC | | | | | | | | ITSLQP | | | | |
| | | | | | | | | | | EDFATY | | | | |
| | | | | | | | | | | YC | | | | |
| ADI-81620 | IGLV3-21 | QHVLTQ | 3622 | GGNNVG | 3815 | WYQQKP | 2283 | GDRDRP | 4096 | GIPERF | 2636 | QVWDSS | 4436 | FGGGTK |
| | | PPSVSV | | STSVH | | GQAPVL | | S | | SGSKSG | | TDHWV | | VTVL |
| | | APGQTA | | | | VVY | | | | NTATLT | | | | |
| | | RVTC | | | | | | | | ISRVEA | | | | |
| | | | | | | | | | | GDEADY | | | | |
| | | | | | | | | | | YC | | | | |
| ADI-81743 | IGKV1D-39 | EIVMTQ | 3623 | RASQSI | 3816 | WYQQRP | 2309 | AASTLQ | 2392 | GVPSRF | 2539 | QQSFST | 4437 | FGQGTK |
| | | SPSSLS | | SSHLS | | GKAPKL | | S | | SGSGSG | | PPT | | VDIK |
| | | ASVRDR | | | | LIY | | | | TDFTLT | | | | |
| | | VTITC | | | | | | | | ITSLQP | | | | |
| | | | | | | | | | | EDFATY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81813 | 2937 | GACATCCGGGTGACCCAGTCTCCATCCTCCCTGTCTGCATCTG | 4733 | DIRVTQSPSSLSASV | 5087 | 6 | 5 |
| | | TAGGAGACAGAGTCACCATCACTTGCCGGGCAAGTCAGGGCAT | | GDRVTITCRASQGIR | | | |
| | | TAGAAATGATTTAGGGTGGTATCAACAACACCCTGGGAAAGCC | | NDLGWYQQHPGKAPK | | | |
| | | CCTAAGCTCCTGATTTCTGCTGCCTCCAGTTTAGAAACTGGGG | | LLISAASSLETGVPS | | | |
| | | TCCCATCAAGGTTCAGCGGCAGTGGATCTGGCACAGATTTCAC | | RFSGSGSGTDFTLTI | | | |
| | | TCTCACCATCACCAGCCTGCAGCCTGAAGATTTTGCAACTTAT | | TSLQPEDFATYYCLQ | | | |
| | | TACTGTCTACAAGATTACAGTAACTTCTGGACGTTCGGCCAAG | | DYSNFWTFGQGTKVE | | | |
| | | GGACCAAGGTGGAGATCAAA | | IK | | | |
| ADI-81890 | 2937 | GAAATGACGATGACGCAGTCTCCTTCCACCCTGTCTGCATCTG | 4734 | EMTMTQSPSTLSASV | 5088 | 6 | 5 |
| | | TAGGAGACAGAGTCACCATCACTTGCCGGGCCAGTCAGAGTAT | | GDRVTITCRASQSIS | | | |
| | | TAGTAACTGGTTGGCCTGGTATCAGCAGAAACCAGGAAAAGCC | | NWLAWYQQKPGKAPK | | | |
| | | CCTAAACTCCTGATCTATATGACATCTCGTTTAGAAGGTGGGG | | LLIYMTSRLEGGVPS | | | |
| | | TCCCATCAAGGTTCAGCGGCAGTGGATCTGGGACAGAATTCAC | | RFSGSGSGTEFTLTI | | | |
| | | TCTCACCATCAGCAGCCTGCAGCCTGATGATTTTGCAACGTAT | | SSLQPDDFATYYCQH | | | |
| | | TACTGTCAGCACTATAATAGTTATTCTGAGATGTACACTTTTG | | YNSYSEMYTFGQGTK | | | |
| | | GCCAGGGGACCAAGGTGGAAATCAAA | | VEIK | | | |
| ADI-81612 | 2939 | CAGCCTGTGGTGACGCAGCCGCCCTCAGTGTCTGGGGCCCCAG | 4735 | QPVVTQPPSVSGAPG | 5089 | 6 | 6 |
| | | GGCAGAGGGTCACCATCTCCTGCACTGGGACCAACTCCAACAT | | QRVTISCTGTNSNIG | | | |
| | | CGGGGGGGGTTATGATGTACACTGGTACCAGCTGCTTCCAGGA | | AGYDVHWYQLLPGTP | | | |
| | | ACACCCCCCAAACTCCTCATCTATCGTAACACCAATCGGCCCT | | PKLLIYRNTNRPSGV | | | |
| | | CAGGGGTCCCTGACCGATTCTCTGGCTCCAAGTCTGGCACCTC | | PDRFSGSKSGTSASL | | | |
| | | AGCCTCCCTGGCCATCACTGGGCTCCAGGCTGAGGATGAGGCT | | AITGLQAEDEADYYC | | | |
| | | GATTATTACTGCCAGTCCTATGACAGCAGCCTGAGTGGTTCGG | | QSYDSSLSGSVFGGG | | | |
| | | TGTTCGGCGGAGGGACCAAGCTGACCGTCCTA | | TKLTVL | | | |
| ADI-81723 | 2944 | GAAACGACACTCACGCAGTCTCCAGACTCCCTGGCTGTGTCTC | 4736 | ETTLTQSPDSLAVSL | 5090 | 6 | 6 |
| | | TGGGCGAGAGGGCCACCATCAACTGCAAGTCCAGCCAGAGTGT | | GERATINCKSSQSVL | | | |
| | | TTTATCCAGCTCCAACAATAGGAACTACTTATCTTGGTACCAG | | SSSNNRNYLSWYQHK | | | |
| | | CACAAACCAGGACAGCCTCCTAAGCTGCTCATTTACTGGGCAT | | PGQPPKLLIYWASTR | | | |
| | | CTACCCGGCAATCCGGGGTCCCTGACCGATTCAGTGGCAGCGG | | QSGVPDRFSGSGSGT | | | |
| | | GTCTGGGACAGATTTCACTCTCACCATCAGCAGCCTGCAACCT | | DFTLTISSLQPEDVA | | | |
| | | GAAGATGTGGCAGTTTATTACTGTCAGCAATATTATAGTTCTC | | VYYCQQYYSSPYTFG | | | |
| | | CCTACACTTTTGGCCAGGGGACCAAAGTGGATATCAAA | | QGTKVDIK | | | |
| ADI-81543 | 2949 | CAGTCTGTGTTGACTCAGCCGCCCTCAGTGTCTGGGGCCCCAG | 4737 | QSVLTQPPSVSGAPG | 5091 | 6 | 5 |
| | | GGCAGAGGGTCACCATCTCCTGCACTGGCAGCAACTCCAACAT | | QRVTISCTGSNSNIG | | | |
| | | CGGGGCAGGTTATGATGTACACTGGTATCAGCAGCTTCCCGGA | | AGYDVHWYQQLPGSA | | | |
| | | TCAGCCCCCAAACTCCTCATCTATGATAACGCCAATCGGCCCT | | PKLLIYDNANRPSGV | | | |
| | | CAGGGGTCCCTGACCGATTCTCTGGCTCCAAGTCTGACACCTC | | PDRFSGSKSDTSASL | | | |
| | | AGCCTCCCTGGCCATCACTGGCCTCCAGGCTGAGGATGAGGCT | | AITGLQAEDEADYYC | | | |
| | | GATTATTACTGCCAGTCCTATGACAGCAGTCTTATTGGTTCTG | | QSYDSSLIGSVFGTG | | | |
| | | TCTTCGGAACTGGGACCAAGCTCACCGTCCTA | | TKLTVL | | | |
| ADI-81583 | 2936 | GACATTGTGATGACCCAGTCTCCATCCTCCCTGTCTGCATCTG | 4738 | DIVMTQSPSSLSASV | 5092 | 6 | 6 |
| | | TGGGAGACAGAGTCACCATCACTTGCCGGGCAAGTCAGAGCGT | | GDRVTITCRASQSVR | | | |
| | | TCGCACCTATTTAAATTGGTATCAGCATAAACCAGGGAAAGCC | | TYLNWYQHKPGKAPK | | | |
| | | CCTAAACTCCTGATCTATGGTGCATCCAGCTTGCAAAGTGGGG | | LLIYGASSLQSGVPS | | | |
| | | TCCCATCAAGGTTCAGTGGCAGTGGATCTGGGACAGATTTCAC | | RFSGSGSGTDFTLTI | | | |
| | | TCTCACCATCACCAGTCTGCAACCTGAAGATTTTGCGACTTAC | | TSLQPEDFATYYCQQ | | | |
| | | TACTGTCAACAGAGTTACAGTACCCCCCCGCTCACTTTCGGCG | | SYSTPPLTFGGGTKV | | | |
| | | GAGGGACCAAGGTGGAAATCAAA | | EIK | | | |
| ADI-81620 | 2947 | CAGCATGTGCTGACTCAGCCACCCTCGGTGTCAGTGGCCCCAG | 4739 | QHVLTQPPSVSVAPG | 5093 | 6 | 6 |
| | | GACAGACGGCCAGGGTTACCTGTGGGGGAAACAACGTTGGGAG | | QTARVTCGGNNVGST | | | |
| | | TACAAGTGTGCACTGGTACCAGCAGAAGCCAGGCCAGGCCCCT | | SVHWYQQKPGQAPVL | | | |
| | | GTGTTGGTCGTCTATGGTGATAGAGACCGGCCCTCAGGGATCC | | VVYGDRDRPSGIPER | | | |
| | | CTGAGCGATTCTCTGGCTCCAAGTCTGGGAACACGGCCACCCT | | FSGSKSGNTATLTIS | | | |
| | | GACCATCAGCAGGGTCGAAGCCGGGGATGAGGCCGACTATTAT | | RVEAGDEADYYCQVW | | | |
| | | TGTCAGGTGTGGGATAGCAGTACTGATCATTGGGTGTTCGGCG | | DSSTDHWVFGGGTKV | | | |
| | | GAGGGACCAAGGTCACCGTCCTA | | TVL | | | |
| ADI-81743 | 2944 | GAAATCGTAATGACCCAGTCTCCATCCTCCCTGTCTGCATCTG | 4740 | EIVMTQSPSSLSASV | 5094 | 6 | 6 |
| | | TAAGAGACAGAGTCACCATCACTTGCCGGGCAAGTCAGAGCAT | | RDRVTITCRASQSIS | | | |
| | | TAGCAGCCATTTAAGTTGGTATCAGCAGAGACCAGGGAAAGCC | | SHLSWYQQRPGKAPK | | | |
| | | CCTAAGCTCCTGATCTATGCTGCATCCACTTTGCAAAGTGGGG | | LLIYAASTLQSGVPS | | | |
| | | TCCCATCAAGGTTCAGTGGCAGTGGATCTGGGACAGATTTCAC | | RFSGSGSGTDFTLTI | | | |
| | | TCTCACCATCACCAGTCTGCAACCTGAAGATTTTGCAACTTAC | | TSLQPEDFATYYCQQ | | | |
| | | TACTGTCAACAGAGTTTCAGTACCCCTCCCACTTTTGGCCAGG | | SFSTPPTFGQGTKVD | | | |
| | | GGACCAAGGTGGATATCAAA | | IK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81795 | IGKV1D-39 | EIVLTQ | 1888 | RASQSI | 2050 | WFQQKP | 2300 | AASTLQ | 2417 | GVPSRF | 4247 | QQSYSS | 2731 | FGGGTK |
| | | SPSSLS | | SSYLN | | GKAPKL | | R | | SGSESG | | PLT | | VEIK |
| | | ASVGDR | | | | LIY | | | | TDFTLT | | | | |
| | | VTITC | | | | | | | | ISSLQP | | | | |
| | | | | | | | | | | EDFANY | | | | |
| | | | | | | | | | | FC | | | | |
| ADI-81812 | IGKV3D-15 | EMTMTQ | 3624 | RASQSV | 3817 | WYQQKP | 2258 | DASTRA | 2424 | GIPARF | 4248 | QQYNDW | 4438 | FGQGTK |
| | | SPGTLS | | DSNLA | | GQAPRL | | T | | SGSGSG | | PPGYT | | VEIK |
| | | VSPGER | | | | LIY | | | | TEFILT | | | | |
| | | ATLSC | | | | | | | | ISSLQS | | | | |
| | | | | | | | | | | EDFAVY | | | | |
| | | | | | | | | | | FC | | | | |
| ADI-81877 | IGLV3-21 | QSVLTQ | 2010 | GGNNIG | 2068 | WYQQKP | 3980 | TNSDRP | 4097 | GIPERF | 4249 | QVWDSA | 4439 | FGGGTK |
| | | PPSVSV | | SRSVH | | GHAPVL | | S | | SGSNSG | | SDQVV | | LTVL |
| | | APGQTA | | | | VVY | | | | NTATLT | | | | |
| | | RITC | | | | | | | | ISRVDA | | | | |
| | | | | | | | | | | GDEADY | | | | |
| | | | | | | | | | | YC | | | | |
| ADI-81577 | IGKV1D-39 | EIVMTQ | 1891 | RASQSI | 2142 | WYQQNP | 3981 | AASRLQ | 2406 | GVPSRF | 4250 | QQSYNI | 4440 | FGGGTK |
| | | SPSSLS | | TTYLN | | GKAPNL | | S | | SGSGSG | | PQLT | | VDIK |
| | | ASVGDR | | | | LIY | | | | TNFTLT | | | | |
| | | VTITC | | | | | | | | ISSLQP | | | | |
| | | | | | | | | | | EDFATY | | | | |
| | | | | | | | | | | YC | | | | |
| ADI-81742 | IGKV1D-39 | EMTMTQ | 3625 | RASQSI | 2077 | WYQQKP | 3942 | DATSLQ | 4098 | GVPSRF | 4251 | QQSYTT | 4441 | FGQGTK |
| | | SPSSLS | | SNYLN | | GKAPKL | | S | | SGSGSG | | WVT | | VEIK |
| | | ASVGDR | | | | LIF | | | | TDFTLT | | | | |
| | | VTIAC | | | | | | | | ISSLQP | | | | |
| | | | | | | | | | | EDFATY | | | | |
| | | | | | | | | | | SC | | | | |
| ADI-81781 | IGKV3D-15 | EMTLTQ | 3626 | RASQSI | 2129 | WYQQKP | 3912 | GASARA | 4099 | GVPARF | 4252 | QHYYDW | 4442 | FGQGTK |
| | | SPGTLS | | SSNLA | | GQAPRL | | T | | SGSGSG | | PPYT | | VEIK |
| | | VSPGER | | | | LIF | | | | TEFTLT | | | | |
| | | ATLSC | | | | | | | | ISSLQS | | | | |
| | | | | | | | | | | EDFAVY | | | | |
| | | | | | | | | | | YC | | | | |
| ADI-81611 | IGKV1-27 | DIQMTQ | 1977 | RASQDI | 3818 | WYQQKP | 3982 | AASTLQ | 2392 | GVPSRF | 4253 | QKYYNV | 4443 | FGQGTK |
| | | SPSSLS | | SNFLA | | GKVPRL | | S | | SGSRSG | | PWT | | VDIK |
| | | ASVGDS | | | | LIY | | | | TDFTLT | | | | |
| | | VTITC | | | | | | | | ITSLQP | | | | |
| | | | | | | | | | | EDVATY | | | | |
| | | | | | | | | | | YC | | | | |
| ADI-81622 | IGKV1D-39 | DIQMTQ | 1878 | RASQSI | 3819 | WYQQKP | 2275 | AASSFQ | 4100 | GVPLRF | 4254 | QQSYSI | 4444 | FGGGTK |
| | | SPSSLS | | SDYLN | | GRAPKL | | S | | TGSGSG | | PLT | | VEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81795 | 2936 | GAAATTGTGTTGACCCAGTCTCCATCCTCCCTGTCTGCATCTG | 4741 | EIVLTQSPSSLSASV | 5095 | 6 | 6 |
| | | TAGGAGACAGAGTCACCATCACTTGCCGGGCAAGTCAGAGCAT | | GDRVTITCRASQSIS | | | |
| | | TAGCAGCTATTTAAATTGGTTTCAGCAGAAACCAGGGAAAGCC | | SYLNWFQQKPGKAPK | | | |
| | | CCTAAGCTCCTGATCTATGCTGCATCCACTTTGCAACGTGGGG | | LLIYAASTLQRGVPS | | | |
| | | TCCCATCAAGGTTCAGTGGCAGTGAATCTGGGACAGATTTCAC | | RFSGSESGTDFTLTI | | | |
| | | TCTCACCATCAGCAGTCTGCAACCGGAAGATTTTGCAAATTAC | | SSLQPEDFANYFCQQ | | | |
| | | TTCTGTCAACAGAGTTACAGTAGCCCGCTCACTTTCGGCGGAG | | SYSSPLTFGGGTKVE | | | |
| | | GGACCAAGGTGGAAATCAAA | | IK | | | |
| ADI-81812 | 2937 | GAAATGACAATGACGCAGTCTCCAGGCACCCTGTCTGTGTCTC | 4742 | EMTMTQSPGTLSVSP | 5096 | 6 | 5 |
| | | CAGGGGAAAGAGCCACCCTCTCCTGCAGGGCCAGTCAGAGTGT | | GERATLSCRASQSVD | | | |
| | | TGACAGCAACTTAGCCTGGTACCAGCAGAAACCTGGCCAGGCT | | SNLAWYQQKPGQAPR | | | |
| | | CCCAGGCTCCTCATCTATGATGCATCCACCAGGGCCACTGGTA | | LLIYDASTRATGIPA | | | |
| | | TCCCAGCCAGGTTCAGTGGCAGTGGGTCTGGGACAGAGTTCAT | | RFSGSGSGTEFILTI | | | |
| | | TCTCACCATCAGCAGCCTGCAGTCTGAAGATTTTGCAGTTTAT | | SSLQSEDFAVYFCQQ | | | |
| | | TTCTGTCAGCAGTATAATGACTGGCCTCCGGGGTACACTTTTG | | YNDWPPGYTFGQGTK | | | |
| | | GCCAGGGGACCAAGGTGGAAATCAAA | | VEIK | | | |
| ADI-81877 | 2939 | CAGTCTGTGCTGACTCAGCCCCCCTCGGTGTCAGTGGCCCCAG | 4743 | QSVLTQPPSVSVAPG | 5097 | 6 | 5 |
| | | GACAGACGGCCAGGATTACCTGTGGGGGAAACAACATTGGAAG | | QTARITCGGNNIGSR | | | |
| | | TAGAAGTGTGCACTGGTACCAGCAGAAGCCAGGCCACGCCCCT | | SVHWYQQKPGHAPVL | | | |
| | | GTGCTGGTCGTCTATACTAATAGCGACCGGCCCTCAGGGATCC | | VVYTNSDRPSGIPER | | | |
| | | CTGAGCGATTCTCTGGCTCCAACTCTGGGAACACGGCCACCCT | | FSGSNSGNTATLTIS | | | |
| | | GACCATCAGCAGGGTCGACGCCGGGGATGAGGCCGACTATTAC | | RVDAGDEADYYCQVW | | | |
| | | TGTCAGGTGTGGGATAGTGCTAGTGATCAGGTGGTATTCGGCG | | DSASDQVVFGGGTKL | | | |
| | | GAGGGACCAAGCTCACCGTCCTA | | TVL | | | |
| ADI-81577 | 2938 | GAAATTGTGATGACCCAGTCTCCATCTTCCCTGTCTGCATCTG | 4744 | EIVMTQSPSSLSASV | 5098 | 6 | 6 |
| | | TTGGAGACAGAGTCACCATCACTTGTCGGGCAAGTCAGAGCAT | | GDRVTITCRASQSIT | | | |
| | | TACCACCTATTTAAATTGGTATCAACAGAATCCAGGGAAAGCC | | TYLNWYQQNPGKAPN | | | |
| | | CCTAACCTCCTGATCTATGCTGCATCCAGGTTGCAGAGTGGGG | | LLIYAASRLQSGVPS | | | |
| | | TCCCATCAAGGTTCAGTGGCAGTGGATCTGGGACAAATTTCAC | | RFSGSGSGTNFTLTI | | | |
| | | TCTCACGATCAGCAGCCTGCAACCCGAAGATTTTGCAACTTAC | | SSLQPEDFATYYCQQ | | | |
| | | TACTGTCAACAGAGTTACAATATTCCTCAACTCACTTTCGGCG | | SYNIPQLTFGGGTKV | | | |
| | | GAGGGACCAAGGTGGATATCAAA | | DIK | | | |
| ADI-81742 | 2937 | GAAATGACAATGACGCAGTCTCCATCCTCCCTGTCTGCATCTG | 4745 | EMTMTQSPSSLSASV | 5099 | 6 | 6 |
| | | TTGGCGACAGAGTCACCATCGCTTGCCGGGCAAGTCAGAGCAT | | GDRVTIACRASQSIS | | | |
| | | CAGCAACTATTTAAATTGGTATCAGCAGAAACCAGGGAAAGCC | | NYLNWYQQKPGKAPK | | | |
| | | CCTAAACTCCTCATCTTTGATGCTACCAGTTTGCAAAGTGGGG | | LLIFDATSLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAACCTGAAGATTTTGCAACTTAC | | SSLQPEDFATYSCQQ | | | |
| | | TCCTGTCAACAGAGTTACACTACCTGGGTCACTTTTGGCCAGG | | SYTTWVTFGQGTKVE | | | |
| | | GGACCAAGGTGGAAATCAAA | | IK | | | |
| ADI-81781 | 2937 | GAAATGACACTGACGCAGTCTCCAGGTACCCTGTCTGTGTCTC | 4746 | EMTLTQSPGTLSVSP | 5100 | 6 | 5 |
| | | CAGGGGAAAGAGCCACCCTCTCTTGCAGGGCCAGTCAGAGTAT | | GERATLSCRASQSIS | | | |
| | | TAGCAGCAACTTAGCCTGGTACCAGCAGAAACCTGGCCAGGCT | | SNLAWYQQKPGQAPR | | | |
| | | CCCAGGCTCCTCATCTTTGGTGCATCCGCCAGGGCCACTGGTG | | LLIFGASARATGVPA | | | |
| | | TCCCAGCCAGGTTCAGTGGCAGTGGGTCTGGGACAGAGTTCAC | | RFSGSGSGTEFTLTI | | | |
| | | TCTCACCATCAGCAGCCTGCAGTCTGAAGATTTTGCAGTTTAT | | SSLQSEDFAVYYCQH | | | |
| | | TACTGTCAGCACTATTATGACTGGCCTCCGTACACTTTTGGCC | | YYDWPPYTFGQGTKV | | | |
| | | AGGGGACCAAGGTGGAAATCAAA | | EIK | | | |
| ADI-81611 | 2944 | GACATCCAGATGACCCAGTCTCCATCCTCCCTGTCTGCATCTG | 4747 | DIQMTQSPSSLSASV | 5101 | 6 | 6 |
| | | TGGGAGACAGCGTCACCATCACTTGCCGGGCGAGTCAGGACAT | | GDSVTITCRASQDIS | | | |
| | | CAGCAATTTTTTAGCCTGGTATCAGCAGAAACCAGGGAAAGTT | | NFLAWYQQKPGKVPR | | | |
| | | CCTAGGCTCCTGATCTATGCTGCATCCACTTTGCAATCAGGGG | | LLIYAASTLQSGVPS | | | |
| | | TCCCATCTCGGTTCAGTGGCAGTAGATCTGGGACAGATTTCAC | | RFSGSRSGTDFTLTI | | | |
| | | TCTCACCATCACCAGCCTGCAGCCTGAAGATGTTGCAACTTAT | | TSLQPEDVATYYCQK | | | |
| | | TACTGTCAAAAGTATTACAATGTCCCTTGGACGTTCGGCCAAG | | YYNVPWTFGQGTKVD | | | |
| | | GGACCAAAGTGGATATCAAA | | IK | | | |
| ADI-81622 | 2936 | GACATCCAGATGACCCAGTCTCCATCCTCCCTGTCTGCATCTG | 4748 | DIQMTQSPSSLSASV | 5102 | 6 | 5 |
| | | TAGGAGACAGAGTCACCATCACTTGCCGGGCAAGTCAGAGCAT | | GDRVTITCRASQSIS | | | |
| | | TAGCGACTATTTAAATTGGTATCAACAAAAACCAGGGAGAGCC | | DYLNWYQQKPGRAPK | | | |
| | | CCTAAGCTCCTCATCTATGCTGCATCCAGTTTTCAAAGTGGGG | | LLIYAASSFQSGVPL | | | |
| | | TCCCATTAAGGTTCACTGGCAGTGGATCTGGGACAGATTTCAC | | RFTGSGSGTDFTLTI | | | |
| | | TCTCACCATCAGCAGTCTGCAACCTGAAGATTTTGCAACTTAC | | SSLQPEDFATYYCQQ | | | |
| | | TACTGTCAACAGAGTTACAGTATCCCGCTCACTTTCGGCGGAG | | SYSIPLTFGGGTKVE | | | |
| | | GGACCAAGGTGGAAATCAAA | | IK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81644 | IGLV3-21 | SYELTQ | 3627 | GGNNIG | 3820 | WYQQKP | 2304 | YHSDRP | 4101 | GFPERF | 4255 | QVWDSG | 4445 | FGGGTK |
| | | PPSVSV | | SKSVY | | GQAPVL | | S | | SGFNSG | | NNHVV | | VTVL |
| | | APGKTA | | | | VIY | | | | STATLT | | | | |
| | | RITC | | | | | | | | ISGVEA | | | | |
| | | | | | | | | | | GDEADY | | | | |
| | | | | | | | | | | YC | | | | |
| ADI-81858 | IGKV1-33 | EIVLTQ | 1888 | QASQDI | 3821 | WYQQKP | 3942 | DVSNLE | 4102 | GVPSRF | 2528 | QHYDNL | 4446 | FGPGTK |
| | | SPSSLS | | DNFLN | | GKAPKL | | T | | SGSGSG | | KFT | | VEIK |
| | | ASVGDR | | | | LIF | | | | TDFTFT | | | | |
| | | VTITC | | | | | | | | ISSLQP | | | | |
| | | | | | | | | | | EDFATY | | | | |
| | | | | | | | | | | YC | | | | |
| ADI-81603 | IGLV1-40 | QSVLTQ | 1948 | TGTNSN | 2171 | WYQRLP | 3983 | VNNNRP | 2471 | GVPDRF | 2599 | QSYDSS | 4383 | FGGGTQ |
| | | PPSVSG | | IGAGYD | | GTAPKL | | S | | SGSKSG | | LSSSL | | LTVL |
| | | APGQRV | | VH | | LIY | | | | TSASLA | | | | |
| | | TISC | | | | | | | | ITGLQA | | | | |
| | | | | | | | | | | EDEADY | | | | |
| | | | | | | | | | | YC | | | | |
| ADI-81535 | IGLV1-40 | QSVLTQ | 1948 | TGSDSN | 3763 | WYQQLP | 2314 | INTNRP | 4103 | GVPDRF | 4256 | QSYDSS | 2840 | FGGGTK |
| | | PPSVSG | | IGAGYD | | GTAPKL | | S | | SGSKSG | | LSDSV | | LTVL |
| | | APGQRV | | VH | | LIY | | | | TSASLA | | | | |
| | | TISC | | | | | | | | ITGLLA | | | | |
| | | | | | | | | | | EDEADY | | | | |
| | | | | | | | | | | YC | | | | |
| ADI-81713 | IGKV3-20 | ETTLTQ | 3628 | RASQSV | 3822 | WYQQKP | 2258 | GSSSRA | 4104 | GIPDRF | 4257 | QQYGTS | 4388 | FGQGTK |
| | | SPGTLS | | RSGYLA | | GQAPRL | | T | | SASGSG | | PWT | | VDIK |
| | | LSPGER | | | | LIY | | | | TDFTLT | | | | |
| | | ATLSC | | | | | | | | ISRLEP | | | | |
| | | | | | | | | | | EDFAVY | | | | |
| | | | | | | | | | | FC | | | | |
| ADI-81816 | IGKV1-5 | EIVMTQ | 3590 | RASQTI | 3823 | WYQQKP | 2257 | MTSRLE | 4105 | GVPSRF | 4241 | QHYNSY | 4447 | FGQGTK |
| | | SPSTLS | | SSWLA | | GKAPKL | | S | | SGSGFG | | FEMYT | | VEIK |
| | | ASVGDR | | | | LIY | | | | TEFTLT | | | | |
| | | VTITC | | | | | | | | ISSLQP | | | | |
| | | | | | | | | | | DDFATY | | | | |
| | | | | | | | | | | YC | | | | |
| ADI-81895 | IGKV3-20 | ETTLTQ | 3629 | RASLSV | 3824 | WYRQKP | 3984 | GASSRA | 2464 | GIPDRF | 2616 | QQYGTS | 4448 | FGQGTR |
| | | SPGTLY | | SAGYLA | | GQAPRL | | T | | SGSGSG | | PIT | | LEIK |
| | | LSPGER | | | | LIY | | | | TDFTLT | | | | |
| | | ATLSC | | | | | | | | ISRLEP | | | | |
| | | | | | | | | | | EDFAVY | | | | |
| | | | | | | | | | | YC | | | | |
| ADI-81793 | IGKV2-24 | EMTLTQ | 3630 | RSSESL | 3825 | WLQQRP | 3985 | KISKRF | 4106 | GVPDRF | 4258 | TQATEF | 4449 | FGQGTK |
| | | SPLSSH | | VHSDGN | | GQPPRL | | S | | IGSGAG | | PQT | | VEIK |
| | | VTLGQP | | TYLN | | LIY | | | | TDFTLK | | | | |
| | | ASISC | | | | | | | | ISRVEA | | | | |
| | | | | | | | | | | EDVGVY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81644 | 2947 | TCGTATGAGCTGACGCAGCCACCCTCAGTGTCAGTGGCCCCAG | 4749 | SYELTQPPSVSVAPG | 5103 | 6 | 6 |
| | | GAAAGACGGCCAGGATTACCTGTGGGGGAAACAACATTGGAAG | | KTARITCGGNNIGSK | | | |
| | | TAAAAGTGTGTACTGGTACCAACAGAAGCCAGGCCAGGCCCCT | | SVYWYQQKPGQAPVL | | | |
| | | GTGCTGGTCATCTACTATCATAGCGACCGGCCCTCAGGGTTCC | | VIYYHSDRPSGFPER | | | |
| | | CTGAGCGATTCTCTGGCTTCAACTCTGGGAGCACGGCCACCCT | | FSGFNSGSTATLTIS | | | |
| | | GACCATCAGCGGGGTCGAAGCCGGGGATGAGGCCGACTATTAC | | GVEAGDEADYYCQVW | | | |
| | | TGTCAGGTCTGGGATAGTGGTAACAATCACGTGGTTTTCGGCG | | DSGNNHVVFGGGTKV | | | |
| | | GAGGCACCAAGGTCACCGTCCTA | | TVL | | | |
| ADI-81858 | 2942 | GAAATCGTGTTGACCCAGTCTCCATCCTCCCTGTCTGCATCTG | 4750 | EIVLTQSPSSLSASV | 5104 | 6 | 5 |
| | | TAGGAGACAGAGTCACCATCACTTGCCAGGCGAGTCAGGACAT | | GDRVTITCQASQDID | | | |
| | | TGACAACTTTTTAAATTGGTATCAGCAAAAACCAGGGAAAGCC | | NFLNWYQQKPGKAPK | | | |
| | | CCTAAACTCCTGATCTTCGATGTATCGAATTTGGAAACAGGGG | | LLIFDVSNLETGVPS | | | |
| | | TCCCATCAAGGTTCAGTGGCAGTGGATCTGGGACAGATTTTAC | | RFSGSGSGTDFTFTI | | | |
| | | TTTCACCATCAGCAGCCTGCAGCCTGAAGATTTTGCAACATAT | | SSLQPEDFATYYCQH | | | |
| | | TACTGTCAACACTATGATAATCTCAAATTCACTTTCGGCCCTG | | YDNLKFTFGPGTKVE | | | |
| | | GGACCAAGGTGGAGATCAAA | | IK | | | |
| ADI-81603 | 2948 | CAGTCTGTCTTGACGCAGCCGCCCTCAGTGTCTGGGGCCCCAG | 4751 | QSVLTQPPSVSGAPG | 5105 | 6 | 5 |
| | | GGCAGAGGGTCACCATCTCCTGCACTGGGACCAACTCCAACAT | | QRVTISCTGTNSNIG | | | |
| | | CGGGGCAGGTTATGATGTACACTGGTACCAGCGACTTCCAGGA | | AGYDVHWYQRLPGTA | | | |
| | | ACAGCCCCCAAACTCCTCATCTATGTTAACAACAATCGGCCCT | | PKLLIYVNNNRPSGV | | | |
| | | CAGGGGTCCCTGACCGATTCTCTGGCTCCAAGTCTGGCACCTC | | PDRFSGSKSGTSASL | | | |
| | | AGCCTCCCTGGCCATCACTGGGCTCCAGGCTGAGGATGAGGCT | | AITGLQAEDEADYYC | | | |
| | | GATTATTACTGCCAGTCCTATGACAGCAGCCTGAGTTCTTCGC | | QSYDSSLSSSLFGGG | | | |
| | | TATTCGGCGGAGGCACCCAGCTGACCGTCCTC | | TQLTVL | | | |
| ADI-81535 | 2939 | CAGTCTGTGCTGACGCAGCCGCCCTCAGTGTCTGGGGCCCCCG | 4752 | QSVLTQPPSVSGAPG | 5106 | 6 | 4 |
| | | GGCAGAGGGTCACCATCTCCTGCACTGGGAGCGACTCCAACAT | | QRVTISCTGSDSNIG | | | |
| | | CGGGGCAGGTTATGATGTACACTGGTACCAGCAGCTTCCAGGA | | AGYDVHWYQQLPGTA | | | |
| | | ACAGCCCCCAAACTCCTCATCTATATTAATACCAATCGGCCCT | | PKLLIYINTNRPSGV | | | |
| | | CAGGGGTCCCTGACCGATTCTCTGGCTCCAAGTCTGGCACCTC | | PDRFSGSKSGTSASL | | | |
| | | AGCCTCCCTGGCCATCACTGGGCTCCTGGCTGAGGATGAGGCT | | AITGLLAEDEADYYC | | | |
| | | GATTATTACTGCCAGTCCTATGACAGCAGCCTGAGTGACTCGG | | QSYDSSLSDSVFGGG | | | |
| | | TATTCGGCGGAGGGACCAAGCTCACCGTCCTA | | TKLTVL | | | |
| ADI-81713 | 2944 | GAAACGACACTCACGCAGTCTCCAGGCACCCTGTCTTTGTCTC | 4753 | ETTLTQSPGTLSLSP | 5107 | 6 | 5 |
| | | CAGGGGAAAGAGCCACCCTCTCCTGCAGGGCCAGTCAGAGTGT | | GERATLSCRASQSVR | | | |
| | | AAGAAGTGGTTACTTAGCCTGGTACCAACAGAAACCTGGCCAG | | SGYLAWYQQKPGQAP | | | |
| | | GCTCCCAGGCTCCTCATCTATGGTTCATCCAGCAGGGCCACTG | | RLLIYGSSSRATGIP | | | |
| | | GCATCCCAGACAGGTTCAGTGCCAGTGGGTCTGGGACAGACTT | | DRFSASGSGTDFTLT | | | |
| | | CACTCTCACCATCAGCAGACTGGAGCCTGAAGATTTTGCAGTG | | ISRLEPEDFAVYFCQ | | | |
| | | TATTTCTGTCAGCAGTATGGTACCTCACCGTGGACGTTCGGCC | | QYGTSPWTFGQGTKV | | | |
| | | AAGGGACCAAAGTGGATATCAAA | | DIK | | | |
| ADI-81816 | 2937 | GAAATTGTGATGACGCAGTCTCCTTCCACCCTGTCTGCATCTG | 4754 | EIVMTQSPSTLSASV | 5108 | 6 | 5 |
| | | TAGGAGACAGAGTCACCATCACTTGCCGGGCCAGTCAGACTAT | | GDRVTITCRASQTIS | | | |
| | | TAGTAGCTGGTTGGCCTGGTATCAGCAGAAACCAGGGAAAGCC | | SWLAWYQQKPGKAPK | | | |
| | | CCTAAGCTCCTGATCTATATGACATCTCGTTTAGAAAGTGGGG | | LLIYMTSRLESGVPS | | | |
| | | TCCCATCAAGGTTCAGCGGCAGTGGATTTGGGACAGAATTCAC | | RFSGSGFGTEFTLTI | | | |
| | | TCTCACCATCAGCAGCCTGCAGCCTGATGATTTTGCAACGTAT | | SSLQPDDFATYYCQH | | | |
| | | TACTGCCAACACTATAATAGTTATTTTGAGATGTACACTTTTG | | YNSYFEMYTFGQGTK | | | |
| | | GCCAGGGGACCAAGGTGGAAATCAAA | | VEIK | | | |
| ADI-81895 | 2940 | GAAACGACACTCACGCAGTCTCCAGGCACCCTGTATTTGTCTC | 4755 | ETTLTQSPGTLYLSP | 5109 | 6 | 5 |
| | | CAGGGGAAAGAGCCACCCTCTCCTGCAGGGCCAGTCTGAGTGT | | GERATLSCRASLSVS | | | |
| | | TAGCGCCGGCTACTTAGCCTGGTACCGGCAGAAACCTGGCCAG | | AGYLAWYRQKPGQAP | | | |
| | | GCTCCCAGGCTCCTCATCTATGGTGCATCCAGCAGGGCCACTG | | RLLIYGASSRATGIP | | | |
| | | GCATCCCAGACAGGTTCAGTGGCAGTGGGTCTGGGACAGACTT | | DRFSGSGSGTDFTLT | | | |
| | | CACTCTCACCATCAGCAGACTGGAGCCTGAAGATTTTGCAGTG | | ISRLEPEDFAVYYCQ | | | |
| | | TATTATTGTCAGCAGTATGGAACCTCACCGATCACCTTCGGCC | | QYGTSPITFGQGTRL | | | |
| | | AAGGGACACGACTGGAGATTAAA | | EIK | | | |
| ADI-81793 | 2937 | GAAATGACACTCACGCAGTCTCCACTCTCCTCACATGTCACCC | 4756 | EMTLTQSPLSSHVTL | 5110 | 5 | 5 |
| | | TTGGACAGCCGGCCTCCATCTCCTGCAGGTCTAGTGAAAGCCT | | GQPASISCRSSESLV | | | |
| | | CGTACACAGTGATGGAAACACCTACTTGAATTGGCTTCAGCAG | | HSDGNTYLNWLQQRP | | | |
| | | AGGCCAGGCCAGCCTCCAAGACTCCTAATCTATAAGATTTCTA | | GQPPRLLIYKISKRF | | | |
| | | AGCGGTTCTCTGGGGTCCCAGACAGATTCATTGGCAGTGGGGC | | SGVPDRFIGSGAGTD | | | |
| | | AGGGACCGATTTCACACTGAAAATCAGCAGGGTGGAAGCTGAG | | FTLKISRVEAEDVGV | | | |
| | | GATGTCGGGGTTTATTACTGCACGCAAGCTACAGAGTTTCCTC | | YYCTQATEFPQTFGQ | | | |
| | | AAACTTTTGGCCAGGGGACCAAGGTGGAGATCAAA | | GTKVEIK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81724 | IGKV1D-39 | EIPMTQ | 1912 | RASQSI | 2050 | WYQQKG | 3986 | AASSLE | 4077 | GVPARF | 4259 | QQSYTT | 2752 | FGQGTK |
| | | SPSSLS | | SSYLN | | GKAPKL | | S | | SGSGSG | | PRT | | VEIK |
| | | ASVGDR | | | | LIY | | | | TDFTLT | | | | |
| | | VTITC | | | | | | | | IRSLQP | | | | |
| | | | | | | | | | | EDFATY | | | | |
| | | | | | | | | | | YC | | | | |
| ADI-81599 | IGKV1D-39 | DIQMTQ | 3631 | RASQSI | 2153 | WYQQKP | 2257 | AASSLQ | 2391 | GVPSRF | 4260 | QQSYST | 2804 | FGQGTR |
| | | SPSSLS | | STFLN | | GKAPKL | | S | | SGSGSG | | PS | | LEIK |
| | | ASVGDR | | | | LIY | | | | TDFTLT | | | | |
| | | VTVTC | | | | | | | | ISGLQP | | | | |
| | | | | | | | | | | EDFAIY | | | | |
| | | | | | | | | | | YC | | | | |
| ADI-81705 | IGKV1-5 | DIGMTQ | 3632 | RASQSI | 2146 | WYQQKA | 3987 | KASTLE | 2518 | GVPSRF | 2532 | QQYNNY | 4450 | FGQGTR |
| | | SPSTLA | | STWLA | | GKAPNL | | S | | SGSGSG | | PIT | | LEIK |
| | | ASVGDR | | | | LIY | | | | TEFTLT | | | | |
| | | VTITC | | | | | | | | ISSLQP | | | | |
| | | | | | | | | | | DDFATY | | | | |
| | | | | | | | | | | YC | | | | |
| ADI-81760 | IGKV3-20 | ETTLTQ | 3628 | RASQSV | 3826 | WYQQKP | 2258 | GASNRA | 4107 | GIPDRF | 2624 | QQYRSP | 4451 | FGGGTK |
| | | SPGTLS | | STTYLA | | GQAPRL | | T | | SGGGSG | | PRLT | | VEIK |
| | | LSPGER | | | | LIY | | | | TDFTLT | | | | |
| | | ATLSC | | | | | | | | ISRLEP | | | | |
| | | | | | | | | | | EDFAVY | | | | |
| | | | | | | | | | | YC | | | | |
| ADI-81843 | IGKV3-11 | ETTLTQ | 3633 | RASQSV | 3827 | WYQQKP | 3988 | DASNRA | 4108 | GIPARF | 2607 | QQRHNR | 4452 | FGGGTK |
| | | SPATLS | | SDYLA | | GQAPRL | | A | | SGSGSG | | LT | | VEIK |
| | | LSPGER | | | | FIS | | | | TDFTLT | | | | |
| | | ATLSC | | | | | | | | ISSLEP | | | | |
| | | | | | | | | | | EDFAVY | | | | |
| | | | | | | | | | | YC | | | | |
| ADI-81887 | IGKV1D-39 | EIPMTQ | 1912 | RASQSI | 3828 | WYQQKP | 2257 | NASSLQ | 4109 | GVPSRF | 2534 | QQSYNT | 2702 | FGQGTK |
| | | SPSSLS | | SRFFN | | GKAPKL | | S | | SGSGSG | | PPWT | | VDIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81580 | IGLV1-40 | QSVLTQ | 3634 | TGSNSN | 2143 | WYQQLP | 2314 | DNINRP | 4110 | GVPDRF | 2622 | QSYDSS | 2840 | FGAGTK |
| | | PPSVSA | | IGAGYD | | GTAPKL | | S | | SGSKSD | | LSDSV | | LTVL |
| | | APGQRV | | VH | | LIY | | | | TSASLA | | | | |
| | | TISC | | | | | | | | ITGLQA | | | | |
| | | | | | | | | | | EDEADY | | | | |
| | | | | | | | | | | YC | | | | |
| ADI-81591 | IGKV1D-39 | DIQMTQ | 1878 | RASQTV | 3829 | WYQQKP | 2257 | AASSLQ | 2391 | GVPSRF | 4240 | QQSYST | 2729 | FGQGTK |
| | | SPSSLS | | SKYLN | | GKAPKL | | S | | SGSGSG | | PRT | | VEIK |
| | | ASVGDR | | | | LIY | | | | TDFTLT | | | | |
| | | VTITQ | | | | | | | | ISSLLP | | | | |
| | | | | | | | | | | EDFATY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81724 | 2937 | GAAATCCCGATGACCCAGTCTCCATCCTCCCTGTCTGCATCTG | 4757 | EIPMTQSPSSLSASV | 5111 | 5 | 4 |
| | | TAGGAGACAGAGTCACCATCACTTGCCGGGCAAGTCAGAGTAT | | GDRVTITCRASQSIS | | | |
| | | TAGTAGTTATTTAAATTGGTATCAACAAAAAGGAGGGAAGGCC | | SYLNWYQQKGGKAPK | | | |
| | | CCTAAACTCCTGATTTATGCTGCATCCAGTTTGGAAAGTGGGG | | LLIYAASSLESGVPA | | | |
| | | TCCCAGCAAGGTTCAGTGGCAGTGGATCTGGCACAGATTTCAC | | RFSGSGSGTDFTLTI | | | |
| | | TCTCACCATCAGAAGTCTGCAACCTGAAGATTTTGCAACTTAC | | RSLQPEDFATYYCQQ | | | |
| | | TACTGTCAACAGAGTTACACTACGCCTCGGACGTTCGGCCAAG | | SYTTPRTFGQGTKVE | | | |
| | | GGACCAAGGTGGAGATCAAA | | IK | | | |
| ADI-81599 | 2940 | GACATCCAAATGACACAGTCTCCATCCTCCCTGTCTGCATCTG | 4758 | DIQMTQSPSSLSASV | 5112 | 5 | 5 |
| | | TAGGAGACAGAGTCACCGTCACTTGCCGGGCAAGTCAGAGTAT | | GDRVTVTCRASQSIS | | | |
| | | AAGCACCTTTTTAAATTGGTATCAACAAAAACCAGGGAAAGCC | | TFLNWYQQKPGKAPK | | | |
| | | CCTAAGCTCCTGATCTATGCTGCATCCAGTTTGCAAAGTGGGG | | LLIYAASSLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCGGTCTGCAACCTGAAGATTTTGCAATTTAT | | SGLQPEDFAIYYCQQ | | | |
| | | TACTGTCAGCAGAGTTACAGTACCCCTTCCTTCGGCCAAGGGA | | SYSTPSFGQGTRLEI | | | |
| | | CACGACTGGAGATTAAA | | K | | | |
| ADI-81705 | 2940 | GATATCGGGATGACGCAGTCTCCTTCCACCCTGGCTGCATCTG | 4759 | DIGMTQSPSTLAASV | 5113 | 5 | 5 |
| | | TAGGAGACAGAGTCACCATTACTTGCCGGGCCAGTCAGAGTAT | | GDRVTITCRASQSIS | | | |
| | | TAGTACCTGGTTGGCCTGGTATCAGCAGAAAGCAGGGAAAGCC | | TWLAWYQQKAGKAPN | | | |
| | | CCGAACCTCCTGATCTATAAGGCGTCTACTTTAGAAAGTGGGG | | LLIYKASTLESGVPS | | | |
| | | TCCCATCAAGGTTCAGCGGCAGTGGATCTGGGACAGAATTCAC | | RFSGSGSGTEFTLTI | | | |
| | | TCTCACCATCAGCAGCCTGCAGCCTGATGATTTTGCGACTTAT | | SSLQPDDFATYYCQQ | | | |
| | | TACTGCCAACAGTATAATAATTACCCTATCACCTTCGGCCAGG | | YNNYPITFGQGTRLE | | | |
| | | GGACACGACTGGAGATTAAA | | IK | | | |
| ADI-81760 | 2936 | GAAACGACACTGACGCAGTCTCCAGGCACCCTGTCTTTGTCTC | 4760 | ETTLTQSPGTLSLSP | 5114 | 5 | 4 |
| | | CAGGGGAAAGAGCCACCCTCTCCTGCAGGGCCAGTCAGAGTGT | | GERATLSCRASQSVS | | | |
| | | TAGCACCACCTACTTAGCCTGGTACCAACAGAAACCTGGCCAG | | TTYLAWYQQKPGQAP | | | |
| | | GCTCCCAGGCTCCTCATCTATGGTGCATCCAATAGGGCCACTG | | RLLIYGASNRATGIP | | | |
| | | GCATCCCAGACAGGTTCAGTGGCGGTGGGTCTGGGACAGACTT | | DRFSGGGSGTDFTLT | | | |
| | | CACTCTCACCATCAGCAGACTGGAGCCTGAAGATTTTGCAGTG | | ISRLEPEDFAVYYCQ | | | |
| | | TATTACTGTCAGCAGTATCGTAGCCCACCTCGGCTCACTTTCG | | QYRSPPRLTFGGGTK | | | |
| | | GCGGAGGGACCAAGGTGGAAATCAAA | | VEIK | | | |
| ADI-81843 | 2936 | GAAACGACACTCACGCAGTCTCCAGCCACCCTGTCTTTGTCTC | 4761 | ETTLTQSPATLSLSP | 5115 | 5 | 4 |
| | | CAGGGGAAAGAGCCACCCTCTCCTGCAGGGCCAGTCAGAGTGT | | GERATLSCRASQSVS | | | |
| | | TAGCGACTACTTAGCCTGGTACCAACAGAAACCTGGCCAGGCT | | DYLAWYQQKPGQAPR | | | |
| | | CCCAGGCTCTTCATTTCTGATGCATCCAACAGGGCCGCTGGCA | | LFISDASNRAAGIPA | | | |
| | | TCCCAGCCAGGTTCAGTGGCAGTGGGTCTGGGACAGACTTCAC | | RFSGSGSGTDFTLTI | | | |
| | | TCTCACCATCAGCAGTCTAGAGCCTGAAGATTTCGCAGTTTAT | | SSLEPEDFAVYYCQQ | | | |
| | | TACTGTCAGCAACGTCACAACCGCCTCACTTTCGGCGGAGGGA | | RHNRLTFGGGTKVEI | | | |
| | | CCAAGGTGGAGATCAAA | | K | | | |
| ADI-81887 | 2944 | GAAATCCCGATGACCCAGTCTCCATCCTCCCTGTCTGCATCTG | 4762 | EIPMTQSPSSLSASV | 5116 | 5 | 4 |
| | | TAGGAGACAGAGTCACCATCACTTGCCGGGCAAGTCAGAGCAT | | GDRVTITCRASQSIS | | | |
| | | TAGCAGGTTTTTTAATTGGTATCAGCAGAAACCAGGGAAAGCC | | RFFNWYQQKPGKAPK | | | |
| | | CCTAAACTCCTGATCTATAATGCATCCAGTTTGCAAAGTGGGG | | LLIYNASSLQSGVPS | | | |
| | | TCCCATCAAGGTTCAGCGGCAGTGGATCTGGGACAGATTTCAC | | RFSGSGSGTDFTLTI | | | |
| | | GCTCACCATCAGCAGTCTGCAACCTGAAGATTTTGCAACTTAC | | SSLQPEDFATYYCQQ | | | |
| | | TACTGTCAACAGAGTTACAATACCCCTCCGTGGACGTTCGGCC | | SYNTPPWTFGQGTKV | | | |
| | | AAGGGACCAAGGTGGATATCAAA | | DIK | | | |
| ADI-81580 | 2958 | CAGTCTGTCCTGACGCAGCCGCCCTCAGTGTCTGCGGCCCCAG | 4763 | QSVLTQPPSVSAAPG | 5117 | 5 | 5 |
| | | GGCAGAGGGTCACCATCTCCTGCACTGGGAGCAACTCCAACAT | | QRVTISCTGSNSNIG | | | |
| | | CGGGGCAGGTTATGATGTACACTGGTACCAGCAACTTCCAGGA | | AGYDVHWYQQLPGTA | | | |
| | | ACAGCCCCCAAACTCCTCATCTATGATAACATCAATCGGCCCT | | PKLLIYDNINRPSGV | | | |
| | | CAGGGGTCCCTGACCGATTCTCTGGCTCCAAGTCTGACACCTC | | PDRFSGSKSDTSASL | | | |
| | | AGCCTCCCTGGCCATCACTGGGCTCCAGGCTGAGGATGAGGCT | | AITGLQAEDEADYYC | | | |
| | | GATTATTACTGCCAGTCCTATGACAGCAGCCTGAGTGATTCTG | | QSYDSSLSDSVFGAG | | | |
| | | TCTTCGGAGCTGGGACCAAGCTCACCGTCCTA | | TKLTVL | | | |
| ADI-81591 | 2937 | GACATCCAGATGACCCAGTCTCCATCCTCCCTGTCTGCATCTG | 4764 | DIQMTQSPSSLSASV | 5118 | 5 | 4 |
| | | TCGGAGACAGAGTCACCATCACTTGCCGGGCAAGCCAGACCGT | | GDRVTITCRASQTVS | | | |
| | | TAGCAAGTATTTAAATTGGTATCAGCAGAAACCAGGGAAAGCC | | KYLNWYQQKPGKAPK | | | |
| | | CCTAAGCTCCTAATCTATGCTGCATCCAGTTTGCAAAGTGGGG | | LLIYAASSLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCTACCTGAAGATTTTGCAACTTAC | | SSLLPEDFATYYCQQ | | | |
| | | TACTGTCAACAGAGTTACAGTACCCCTCGGACTTTTGGCCAGG | | SYSTPRTFGQGTKVE | | | |
| | | GGACCAAGGTGGAAATCAAA | | IK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81731 | IGLV2-8 | QSVLTQ | 3582 | TGSSSD | 3830 | WYQQHP | 3989 | EVTKRP | 2449 | GVPDRF | 4261 | SSYAGS | 4453 | FGGGTK |
| | | PPSASG | | VGGYNY | | GKAPKL | | S | | SGSKSG | | NIVV | | LTVL |
| | | SPGQSV | | VS | | IIF | | | | NTASLT | | | | |
| | | TISC | | | | | | | | VSGLQA | | | | |
| | | | | | | | | | | EDEANY | | | | |
| | | | | | | | | | | YC | | | | |
| ADI-81750 | IGKV1-33 | EIPMTQ | 1912 | QASQHI | 3831 | WYQQKP | 2333 | DASNLE | 2416 | GVPSRF | 4262 | QHSRT | 4454 | FGPGTK |
| | | SPSSLS | | NNYLN | | GKAPKL | | T | | SGSRSG | | | | VEIK |
| | | ASVGDR | | | | LIS | | | | TDFSFT | | | | |
| | | VTITC | | | | | | | | ISSLQP | | | | |
| | | | | | | | | | | EDIATY | | | | |
| | | | | | | | | | | YC | | | | |
| ADI-81830 | IGLV1-44 | QSVLTQ | 3566 | SGSNSN | 3832 | WYQKLP | 3990 | TNNQRP | 4111 | GVPDRF | 2615 | AAWDDS | 4455 | FGGGTK |
| | | PPSASG | | IGSNTV | | GTAPKL | | S | | SGSKSG | | LSGHVV | | VTVL |
| | | TPGQRV | | S | | LIS | | | | TSASLA | | | | |
| | | TISC | | | | | | | | ISGLQS | | | | |
| | | | | | | | | | | EDEADY | | | | |
| | | | | | | | | | | YC | | | | |
| ADI-81774 | IGKV1-33 | DIRMTQ | 1937 | QASQDI | 3833 | WYQQKP | 2257 | DASSLE | 2434 | GVPSRF | 4263 | QQYDNL | 4456 | FGQGTK |
| | | SPSSLS | | TTYLN | | GKAPKL | | S | | SGSGSG | | PYT | | VDIK |
| | | ASVGDR | | | | LIY | | | | TDFTFT | | | | |
| | | VTITC | | | | | | | | ISRLQP | | | | |
| | | | | | | | | | | EDIATY | | | | |
| | | | | | | | | | | YC | | | | |
| ADI-81804 | IGLV1-44 | SYELTQ | 1962 | SGSSSN | 2217 | WYQQLP | 3991 | NKNRRP | 4112 | GVPDRF | 4264 | AAWDDS | 2813 | FGGGTK |
| | | PPSASG | | IGSNTV | | GTAPKL | | S | | SASKSG | | LNGVV | | LTVL |
| | | TPGQRV | | N | | FIY | | | | TSASLA | | | | |
| | | TISC | | | | | | | | ISGLQS | | | | |
| | | | | | | | | | | EDEADY | | | | |
| | | | | | | | | | | YC | | | | |
| ADI-81544 | IGLV1-40 | QSVLTQ | 1948 | TGSNSN | 2143 | WYQQLP | 3992 | VNSNRP | 4113 | GVPDRF | 4265 | QSYDSS | 2911 | FGGGTK |
| | | PPSVSG | | IGAGYD | | GTAPKL | | S | | SGSRSG | | LSGSL | | LTVL |
| | | APGQRV | | VH | | LIS | | | | TSAFLA | | | | |
| | | TISC | | | | | | | | ITGLQA | | | | |
| | | | | | | | | | | EDEADY | | | | |
| | | | | | | | | | | YC | | | | |
| ADI-81691 | IGKV1D-39 | EIPMTQ | 1912 | RASQSI | 2050 | WYQQKP | 2257 | AASKLH | 4114 | GVPSRF | 2626 | QQSYSS | 4384 | FGPGTK |
| | | SPSSLS | | SSYLN | | GKAPKL | | G | | RGSGSG | | PPWT | | VDIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81571 | IGLV1-40 | QSVLTQ | 1948 | TGSNSN | 2143 | WYQQLP | 2331 | VNNNRP | 2471 | GVPDRF | 2622 | QSYDSS | 2826 | FGGGTK |
| | | PPSVSG | | IGAGYD | | GRAPKL | | S | | SGSKSD | | LSASV | | VTVL |
| | | APGQRV | | IVH | | ILIY | | | | TSASLA | | | | |
| | | TISC | | | | | | | | ITGLQA | | | | |
| | | | | | | | | | | EDEADY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81731 | 2939 | CAGTCTGTGCTGACTCAGCCTCCCTCCGCGTCCGGGTCTCCTG | 4765 | QSVLTQPPSASGSPG | 5119 | 5 | 5 |
| | | GACAGTCAGTCACCATCTCCTGCACTGGAAGCAGCAGTGACGT | | QSVTISCTGSSSDVG | | | |
| | | TGGTGGTTACAACTATGTCTCCTGGTACCAACAACACCCAGGC | | GYNYVSWYQQHPGKA | | | |
| | | AAAGCCCCCAAACTCATCATTTTTGAGGTCACTAAGCGGCCCT | | PKLIIFEVTKRPSGV | | | |
| | | CAGGGGTCCCTGATCGTTTCTCTGGCTCCAAGTCTGGCAACAC | | PDRFSGSKSGNTASL | | | |
| | | GGCCTCCCTGACCGTCTCTGGGCTCCAGGCTGAGGATGAGGCT | | TVSGLQAEDEANYYC | | | |
| | | AATTATTACTGCAGCTCATATGCAGGCAGCAACATTGTGGTAT | | SSYAGSNIVVFGGGT | | | |
| | | TCGGCGGAGGGACCAAGCTCACCGTCCTA | | KLTVL | | | |
| ADI-81750 | 2942 | GAAATCCCAATGACCCAGTCTCCATCCTCCCTGTCTGCATCTG | 4766 | EIPMTQSPSSLSASV | 5120 | 5 | 5 |
| | | TAGGAGACAGAGTCACCATCACTTGCCAGGCGAGTCAGCACAT | | GDRVTITCQASQHIN | | | |
| | | AAACAACTATTTAAATTGGTATCAGCAGAAACCAGGGAAAGCC | | NYLNWYQQKPGKAPK | | | |
| | | CCTAAGCTCCTGATCTCCGATGCATCCAATTTGGAAACAGGGG | | LLISDASNLETGVPS | | | |
| | | TCCCATCAAGATTCAGTGGAAGTAGATCTGGGACAGATTTTAG | | RFSGSRSGTDFSFTI | | | |
| | | TTTCACCATCAGCAGCCTGCAGCCTGAAGATATTGCAACATAT | | SSLQPEDIATYYCQH | | | |
| | | TACTGTCAACACTCAAGGACTTTCGGCCCTGGGACCAAGGTGG | | SRTFGPGTKVEIK | | | |
| | | AAATCAAA | | | | | |
| ADI-81830 | 2947 | CAGTCTGTGCTGACTCAGCCACCCTCAGCGTCTGGGACCCCCG | 4767 | QSVLTQPPSASGTPG | 5121 | 5 | 5 |
| | | GGCAGAGGGTCACCATCTCTTGCTCTGGAAGCAACTCCAACAT | | QRVTISCSGSNSNIG | | | |
| | | CGGAAGTAATACTGTAAGCTGGTACCAGAAGCTCCCAGGAACG | | SNTVSWYQKLPGTAP | | | |
| | | GCCCCCAAACTCCTCATCTCTACTAATAATCAGCGGCCCTCAG | | KLLISTNNQRPSGVP | | | |
| | | GGGTCCCTGACCGATTCTCTGGCTCCAAGTCTGGCACCTCAGC | | DRFSGSKSGTSASLA | | | |
| | | CTCCCTGGCCATCAGTGGGCTCCAGTCTGAGGATGAGGCTGAT | | ISGLQSEDEADYYCA | | | |
| | | TATTACTGTGCAGCATGGGATGACAGCCTGAGTGGTCATGTGG | | AWDDSLSGHVVFGGG | | | |
| | | TATTCGGCGGAGGGACCAAGGTCACCGTCCTA | | TKVTVL | | | |
| ADI-81774 | 2944 | GACATCCGGATGACCCAGTCTCCATCCTCCCTGTCTGCATCTG | 4768 | DIRMTQSPSSLSASV | 5122 | 5 | 5 |
| | | TAGGAGACAGAGTCACCATCACTTGCCAGGCGAGTCAGGACAT | | GDRVTITCQASQDIT | | | |
| | | TACCACCTATTTAAATTGGTATCAGCAGAAACCAGGGAAAGCC | | TYLNWYQQKPGKAPK | | | |
| | | CCTAAGCTCCTGATCTACGATGCATCCAGTTTGGAATCAGGGG | | LLIYDASSLESGVPS | | | |
| | | TCCCATCAAGGTTCAGTGGAAGTGGATCTGGGACAGATTTTAC | | RFSGSGSGTDFTFTI | | | |
| | | TTTCACCATCAGCAGACTGCAGCCTGAAGATATTGCAACATAT | | SRLQPEDIATYYCQQ | | | |
| | | TACTGTCAACAATATGATAATCTCCCGTACACTTTTGGCCAGG | | YDNLPYTFGQGTKVD | | | |
| | | GGACCAAGGTGGATATCAAA | | IK | | | |
| ADI-81804 | 2939 | TCGTATGAGCTGACTCAGCCACCCTCAGCGTCTGGGACCCCCG | 4769 | SYELTQPPSASGTPG | 5123 | 5 | 5 |
| | | GGCAGAGGGTCACCATCTCTTGTTCTGGAAGCAGCTCCAACAT | | QRVTISCSGSSSNIG | | | |
| | | CGGAAGTAATACTGTGAACTGGTACCAGCAGCTCCCAGGAACG | | SNTVNWYQQLPGTAP | | | |
| | | GCCCCCAAACTCTTCATTTATAATAAGAATCGGCGGCCCTCAG | | KLFIYNKNRRPSGVP | | | |
| | | GGGTCCCTGACCGATTCTCTGCCTCCAAGTCTGGCACCTCTGC | | DRFSASKSGTSASLA | | | |
| | | CTCCCTGGCCATCAGTGGGCTCCAGTCTGAGGATGAGGCTGAT | | ISGLQSEDEADYYCA | | | |
| | | TATTACTGTGCAGCCTGGGATGACAGCCTGAATGGTGTGGTGT | | AWDDSLNGVVFGGGT | | | |
| | | TCGGCGGAGGGACCAAGCTCACCGTCCTA | | KLTVL | | | |
| ADI-81544 | 2939 | CAGTCTGTGTTGACGCAGCCGCCCTCAGTGTCTGGGGCCCCTG | 4770 | QSVLTQPPSVSGAPG | 5124 | 5 | 5 |
| | | GGCAGAGGGTCACCATCTCCTGCACTGGGAGCAACTCCAACAT | | QRVTISCTGSNSNIG | | | |
| | | CGGGGCAGGCTATGATGTACACTGGTACCAGCAACTTCCAGGG | | AGYDVHWYQQLPGTA | | | |
| | | ACAGCCCCCAAACTCCTCATCTCTGTTAACAGCAATCGGCCCT | | PKLLISVNSNRPSGV | | | |
| | | CAGGGGTCCCTGACCGATTCTCTGGCTCCAGGTCTGGCACCTC | | PDRFSGSRSGTSAFL | | | |
| | | AGCCTTCCTGGCCATCACTGGGCTCCAGGCTGAGGATGAGGCT | | AITGLQAEDEADYYC | | | |
| | | GATTATTACTGCCAGTCCTATGACAGCAGTCTCAGTGGTTCGC | | QSYDSSLSGSLFGGG | | | |
| | | TATTCGGCGGAGGCACCAAGCTGACCGTCCTA | | TKLTVL | | | |
| ADI-81691 | 2950 | GAAATCCCGATGACCCAGTCTCCATCCTCCCTGTCTGCATCAG | 4771 | EIPMTQSPSSLSASV | 5125 | 5 | 4 |
| | | TAGGAGACAGAGTCACCATCACTTGCCGGGCAAGTCAGAGCAT | | GDRVTITCRASQSIS | | | |
| | | TAGCAGCTATTTAAATTGGTATCAGCAGAAACCAGGGAAAGCC | | SYLNWYQQKPGKAPK | | | |
| | | CCTAAGCTCCTGATCTATGCTGCATCCAAATTGCACGGTGGGG | | LLIYAASKLHGGVPS | | | |
| | | TCCCATCAAGGTTCAGAGGCAGTGGATCTGGGACAGATTTCAC | | RFRGSGSGTDFTLTI | | | |
| | | TCTCACCATCAGCAGTCTGCAACCTGAAGATTTTGCAACTTAC | | SSLQPEDFATYYCQQ | | | |
| | | TACTGTCAACAGAGTTACAGCTCCCCTCCGTGGACGTTCGGCC | | SYSSPPWTFGPGTKV | | | |
| | | CAGGGACCAAGGTGGATATCAAA | | DIK | | | |
| ADI-81571 | 2947 | CAGTCTGTGTTGACGCAGCCGCCCTCAGTGTCTGGGGCCCCAG | 4772 | QSVLTQPPSVSGAPG | 5126 | 5 | 5 |
| | | GGCAGAGGGTCACCATCTCCTGCACTGGGAGCAACTCCAACAT | | QRVTISCTGSNSNIG | | | |
| | | CGGGGCAGGTTATGATGTGCACTGGTACCAGCAGCTTCCAGGA | | AGYDVHWYQQLPGRA | | | |
| | | AGAGCCCCCAAACTCCTCATCTATGTTAACAACAATCGGCCCT | | PKLLIYVNNNRPSGV | | | |
| | | CAGGGGTCCCTGACCGATTCTCTGGCTCCAAGTCTGACACCTC | | PDRFSGSKSDTSASL | | | |
| | | CGCCTCCCTGGCCATCACTGGGCTCCAGGCTGAGGATGAGGCT | | AITGLQAEDEADYYC | | | |
| | | GACTATTACTGCCAGTCCTATGACAGCAGCCTGAGTGCCTCGG | | QSYDSSLSASVFGGG | | | |
| | | TTTTCGGCGGAGGGACCAAGGTCACCGTCCTA | | TKVTVL | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81850 | IGKV1D-39 | EMTLTQ | 1914 | RASQSI | 3834 | WYQQKP | 2257 | AASTLH | 4115 | GVPSRF | 2692 | QQSYSN | 4457 | FGQGTR |
| | | SPSSLS | | SSYLH | | GKAPKL | | T | | SGGGSG | | PQVT | | LEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81628 | IGKV1D-39 | DIVMTQ | 1902 | RASQSV | 2234 | WYQQKP | 2263 | AASSLQ | 2391 | GVPSRF | 4266 | QQSYTT | 2833 | FGQGTK |
| | | SPSSLS | | SNYLN | | GKAPNL | | S | | SGSGSG | | PYT | | VEIK |
| | | ASVGDR | | | | LIY | | | | TDFTLT | | | | |
| | | VTITC | | | | | | | | ITSLQP | | | | |
| | | | | | | | | | | EDFATY | | | | |
| | | | | | | | | | | HC | | | | |
| ADI-81678 | IGKV1-5 | DIVMTQ | 3635 | RASQSI | 3812 | WYQQKP | 3993 | RASSLE | 4116 | GVPSRF | 4267 | QQSNSP | 4458 | FGQGTK |
| | | SPSTLS | | SNWLA | | GKVPKL | | S | | SGSRSG | | WT | | VEIK |
| | | ASVGDR | | | | LIS | | | | TEFTLT | | | | |
| | | VTITQ | | | | | | | | ISSLQP | | | | |
| | | | | | | | | | | DDFATY | | | | |
| | | | | | | | | | | YC | | | | |
| ADI-81738 | IGKV1D-39 | EIRLTQ | 3636 | RASQSI | 3816 | WYQQRP | 2309 | AASSLQ | 4117 | GVPSRF | 2534 | QQSFST | 4437 | FGQGTK |
| | | SPSSLS | | SSHLS | | GKAPKL | | T | | SGSGSG | | PPT | | VDIK |
| | | ASVRDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81667 | IGKV1-5 | DIRMTQ | 3610 | RASQSI | 2146 | WYQQKP | 3994 | KASTLK | 4118 | GVPSRF | 2532 | QQYNTY | 4459 | FGQGTK |
| | | SPSTLS | | STWLA | | GKAPNV | | S | | SGSGSG | | SEA | | VEIK |
| | | ASVGDR | | | | LIY | | | | TEFTLT | | | | |
| | | VTITC | | | | | | | | ISSLQP | | | | |
| | | | | | | | | | | DDFATY | | | | |
| | | | | | | | | | | YC | | | | |
| ADI-81832 | IGKV1-33 | EIVMTQ | 1891 | QASQDI | 3835 | WYQQKP | 2316 | DASNLE | 2397 | GVPSRF | 4268 | QQYDNL | 2700 | FGGGTK |
| | | SPSSLS | | RNYLN | | GKAPKV | | A | | SGSGSG | | PLT | | VEIK |
| | | ASVGDR | | | | LIY | | | | TDFSFT | | | | |
| | | VTITC | | | | | | | | ISSLQP | | | | |
| | | | | | | | | | | EDIATY | | | | |
| | | | | | | | | | | YC | | | | |
| ADI-81711 | IGKV1-33 | EITMTQ | 3637 | QASQDI | 3835 | WYQQKP | 2316 | AASDLE | 4119 | GVPSRF | 4269 | QQYDNL | 4460 | FGQGTK |
| | | SPSSLS | | RNYLN | | GKAPKV | | T | | SGSGSG | | YT | | VEIK |
| | | ASVGDR | | | | LIY | | | | TDFTLT | | | | |
| | | VTITQ | | | | | | | | ISSLQP | | | | |
| | | | | | | | | | | EDIATY | | | | |
| | | | | | | | | | | YC | | | | |
| ADI-81762 | IGKV1D-39 | DIPMTQ | 1947 | RASQSI | 2088 | WYQQKP | 3995 | AASTLQ | 2392 | GVPSRF | 4270 | QQTYIP | 4461 | FGQGTK |
| | | SPSSLS | | NSYLN | | GKAPKF | | S | | SGSGSE | | PRT | | VEIK |
| | | ASVGDR | | | | LIY | | | | THFTLT | | | | |
| | | VTITQ | | | | | | | | ISSLQP | | | | |
| | | | | | | | | | | EDFATY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81850 | 2940 | GAAATGACACTGACCCAGTCTCCATCCTCCCTGTCTGCATCTG | 4773 | EMTLTQSPSSLSASV | 5127 | 5 | 5 |
| | | TAGGAGACAGAGTCACAATCACTTGCCGGGCAAGTCAGAGCAT | | GDRVTITCRASQSIS | | | |
| | | TAGCAGCTATTTACATTGGTATCAGCAGAAACCAGGGAAAGCC | | SYLHWYQQKPGKAPK | | | |
| | | CCTAAGCTCCTGATCTATGCTGCATCCACTTTGCACACTGGGG | | LLIYAASTLHTGVPS | | | |
| | | TCCCATCAAGGTTCAGTGGCGGTGGATCTGGGACAGATTTCAC | | RFSGGGSGTDFTLTI | | | |
| | | TCTCACCATCAGCAGTCTGCAACCTGAAGATTTTGCAACTTAC | | SSLQPEDFATYYCQQ | | | |
| | | TACTGTCAACAGAGTTACAGTAACCCTCAGGTCACCTTCGGCC | | SYSNPQVTFGQGTRL | | | |
| | | AAGGGACACGACTGGAGATTAAA | | EIK | | | |
| ADI-81628 | 2937 | GACATTGTGATGACCCAGTCTCCATCCTCCCTGTCTGCGTCTG | 4774 | DIVMTQSPSSLSASV | 5128 | 5 | 5 |
| | | TAGGAGACAGAGTCACCATCACTTGCCGGGCAAGTCAGAGCGT | | GDRVTITCRASQSVS | | | |
| | | TAGCAACTATTTAAATTGGTATCAGCAGAAACCAGGGAAAGCC | | NYLNWYQQKPGKAPN | | | |
| | | CCTAACCTCCTGATCTATGCTGCATCCAGTTTGCAAAGTGGGG | | LLIYAASSLQSGVPS | | | |
| | | TCCCATCAAGGTTCAGTGGCAGTGGATCTGGGACAGACTTCAC | | RFSGSGSGTDFTLTI | | | |
| | | TCTCACCATCACCAGTCTGCAACCTGAAGATTTTGCAACTTAC | | TSLQPEDFATYHCQQ | | | |
| | | CACTGTCAGCAGAGTTACACTACCCCGTACACTTTTGGCCAGG | | SYTTPYTFGQGTKVE | | | |
| | | GGACCAAGGTGGAAATCAAA | | IK | | | |
| ADI-81678 | 2937 | GATATTGTGATGACCCAGTCTCCTTCCACCCTGTCTGCATCTG | 4775 | DIVMTQSPSTLSASV | 5129 | 5 | 5 |
| | | TAGGAGACAGAGTCACCATCACTTGCCGGGCCAGTCAGAGTAT | | GDRVTITCRASQSIS | | | |
| | | TAGTAACTGGTTGGCCTGGTATCAGCAGAAACCAGGGAAAGTC | | NWLAWYQQKPGKVPK | | | |
| | | CCTAAACTCCTGATCTCTAGGGCGTCTAGTTTAGAAAGTGGGG | | LLISRASSLESGVPS | | | |
| | | TCCCATCAAGGTTCAGCGGCAGTAGATCTGGGACAGAATTCAC | | RFSGSRSGTEFTLTI | | | |
| | | TCTCACCATCAGCAGCCTGCAGCCTGATGATTTTGCAACTTAT | | SSLQPDDFATYYCQQ | | | |
| | | TACTGCCAACAGAGTAATAGTCCGTGGACGTTCGGCCAAGGGA | | SNSPWTFGQGTKVEI | | | |
| | | CCAAGGTGGAGATCAAA | | K | | | |
| ADI-81738 | 2944 | GAAATCCGGTTGACCCAGTCTCCATCCTCCCTGTCTGCATCTG | 4776 | EIRLTQSPSSLSASV | 5130 | 5 | 5 |
| | | TAAGAGACAGAGTCACCATCACTTGCCGGGCAAGTCAGAGCAT | | RDRVTITCRASQSIS | | | |
| | | TAGCAGCCATTTAAGTTGGTATCAGCAGAGACCAGGGAAAGCC | | SHLSWYQQRPGKAPK | | | |
| | | CCTAAGCTCCTGATCTATGCTGCATCCAGTTTGCAAACTGGGG | | LLIYAASSLQTGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAACCTGAAGATTTTGCAACTTAC | | SSLQPEDFATYYCQQ | | | |
| | | TACTGTCAACAGAGTTTCAGTACCCCTCCCACTTTTGGCCAGG | | SFSTPPTFGQGTKVD | | | |
| | | GGACCAAGGTGGATATCAAA | | IK | | | |
| ADI-81667 | 2937 | GACATCCGGATGACCCAGTCTCCTTCCACCCTGTCTGCATCTG | 4777 | DIRMTQSPSTLSASV | 5131 | 5 | 5 |
| | | TAGGAGACAGAGTCACCATCACTTGCCGGGCCAGTCAGAGTAT | | GDRVTITCRASQSIS | | | |
| | | TAGTACCTGGTTGGCCTGGTATCAGCAGAAACCAGGGAAAGCC | | TWLAWYQQKPGKAPN | | | |
| | | CCTAATGTCCTGATCTATAAGGCGTCTACTTTAAAAAGTGGGG | | VLIYKASTLKSGVPS | | | |
| | | TCCCATCAAGGTTCAGCGGCAGTGGATCTGGGACAGAATTCAC | | RFSGSGSGTEFTLTI | | | |
| | | TCTCACCATCAGCAGCCTGCAGCCTGATGATTTTGCAACTTAT | | SSLQPDDFATYYCQQ | | | |
| | | TACTGCCAACAGTATAATACTTATTCTGAAGCGTTCGGCCAAG | | YNTYSEAFGQGTKVE | | | |
| | | GGACCAAGGTGGAGATCAAA | | IK | | | |
| ADI-81832 | 2936 | GAGATTGTGATGACCCAGTCTCCATCCTCCCTGTCTGCATCTG | 4778 | EIVMTQSPSSLSASV | 5132 | 5 | 4 |
| | | TAGGAGACAGAGTCACCATCACTTGCCAGGCGAGTCAGGACAT | | GDRVTITCQASQDIR | | | |
| | | TAGAAACTATTTAAATTGGTATCAGCAGAAACCAGGGAAAGCC | | NYLNWYQQKPGKAPK | | | |
| | | CCTAAGGTGCTGATCTACGATGCATCCAATTTGGAAGCAGGGG | | VLIYDASNLEAGVPS | | | |
| | | TCCCATCAAGATTCAGTGGAAGTGGATCTGGGACAGATTTTTC | | RFSGSGSGTDFSFTI | | | |
| | | TTTCACCATCAGCAGCCTGCAGCCTGAAGATATTGCAACATAT | | SSLQPEDIATYYCQQ | | | |
| | | TACTGTCAACAGTATGATAATCTTCCGCTCACTTTCGGCGGAG | | YDNLPLTFGGGTKVE | | | |
| | | GGACCAAGGTGGAGATCAAA | | IK | | | |
| ADI-81711 | 2937 | GAAATCACAATGACCCAGTCTCCATCCTCCCTGTCTGCATCTG | 4779 | EITMTQSPSSLSASV | 5133 | 5 | 5 |
| | | TAGGAGACAGAGTCACCATCACTTGCCAGGCGAGTCAGGACAT | | GDRVTITCQASQDIR | | | |
| | | TAGGAACTATTTAAATTGGTATCAGCAGAAACCAGGGAAAGCC | | NYLNWYQQKPGKAPK | | | |
| | | CCTAAGGTCCTGATCTACGCTGCATCCGATTTGGAAACAGGGG | | VLIYAASDLETGVPS | | | |
| | | TCCCGTCAAGGTTCAGTGGAAGTGGATCTGGGACAGATTTTAC | | RFSGSGSGTDFTLTI | | | |
| | | TCTCACCATCAGCAGCCTGCAGCCTGAAGATATTGCAACATAT | | SSLQPEDIATYYCQQ | | | |
| | | TACTGTCAACAGTATGATAATCTTTACACTTTTGGCCAGGGGA | | YDNLYTFGQGTKVEI | | | |
| | | CCAAGGTGGAAATCAAA | | K | | | |
| ADI-81762 | 2937 | GACATCCCGATGACCCAGTCTCCATCCTCCCTGTCTGCATCTG | 4780 | DIPMTQSPSSLSASV | 5134 | 5 | 5 |
| | | TAGGAGACAGAGTCACCATCACTTGCCGGGCAAGTCAGAGCAT | | GDRVTITCRASQSIN | | | |
| | | TAACAGCTATTTAAATTGGTATCAGCAGAAACCAGGGAAAGCC | | SYLNWYQQKPGKAPK | | | |
| | | CCTAAGTTCCTGATCTATGCTGCCTCCACTCTTCAAAGTGGGG | | FLIYAASTLQSGVPS | | | |
| | | TCCCATCACGCTTCAGTGGCAGTGGATCTGAGACACATTTCAC | | RFSGSGSETHFTLTI | | | |
| | | TCTCACCATCAGCAGTCTGCAACCTGAAGATTTTGCAACTTAC | | SSLQPEDFATYYCQQ | | | |
| | | TACTGTCAACAGACTTACATTCCCCCTCGAACGTTCGGCCAAG | | TYIPPRTFGQGTKVE | | | |
| | | GGACCAAGGTGGAAATCAAA | | IK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81626 | IGKV4-1 | DIPMTQ | 1978 | KTSQSV | 3836 | WYQQKP | 2290 | WASTRE | 2422 | GVPDRF | 2619 | QQCSVT | 4462 | FGQGTK |
| | | SPDSLA | | LDRSDN | | GQPPKL | | S | | SGSGSG | | PPMCS | | VEIK |
| | | VSLGER | | RNYLA | | LIY | | | | TDFTLT | | | | |
| | | ATINC | | | | | | | | ISSLQA | | | | |
| | | | | | | | | | | EDVAVY | | | | |
| | | | | | | | | | | YC | | | | |
| ADI-81803 | IGKV1D-39 | DIQMTQ | 1878 | RASQNI | 3837 | WYQQKP | 2316 | AASSLQ | 2391 | GVPSRF | 2551 | QQSYSS | 4463 | FGQGTK |
| | | SPSSLS | | NTYLN | | GKAPKV | | S | | TGSGSG | | PRT | | VEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81882 | IGKV1-33 | DITLTQ | 3638 | QASQDI | 3838 | WYQQKP | 3942 | DVSNLE | 4102 | GVPSRF | 2537 | QHYDNL | 4446 | FGPGTK |
| | | SPSSLS | | NNFLN | | GKAPKL | | T | | SGSGSG | | KFT | | VEIK |
| | | ASVGDR | | | | LIF | | | | TDFTFT | | | | |
| | | VTFTC | | | | | | | | ISSLQP | | | | |
| | | | | | | | | | | EDIATY | | | | |
| | | | | | | | | | | YC | | | | |
| ADI-81911 | IGKV1D-39 | EIVMTQ | 1891 | RASQSI | 3839 | WYQQKP | 2257 | GASSLQ | 2403 | GVPSRI | 4271 | QQSYST | 4464 | FGGGTK |
| | | SPSSLS | | YNYLN | | GKAPKL | | S | | SGSGSG | | PGT | | VDIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81557 | IGKV3-20 | EIVLTQ | 3581 | RASQSV | 2173 | WYQQKP | 3996 | DASSRA | 4120 | GIPDRF | 4272 | QQYGSS | 4465 | FGQGTK |
| | | SPGTLS | | SSNYLA | | GQAPRL | | A | | SGSGSG | | PGS | | VEIK |
| | | LSPGER | | | | LIS | | | | TDFTLT | | | | |
| | | ATLSC | | | | | | | | IGRLEP | | | | |
| | | | | | | | | | | EDFAVY | | | | |
| | | | | | | | | | | YC | | | | |
| ADI-81886 | IGKV4-1 | DMTLTQ | 3639 | KSSQSV | 3840 | WYQQKP | 3997 | WASTRE | 2422 | GVPDRF | 4273 | QQSYSP | 4466 | FGQGTK |
| | | SPDSLA | | LSSSNN | | GRPPKL | | S | | SGGGSG | | PYT | | VEIK |
| | | VSLGER | | KNYLT | | LIY | | | | TDFTLS | | | | |
| | | ATINC | | | | | | | | ISSLQA | | | | |
| | | | | | | | | | | EDVAVY | | | | |
| | | | | | | | | | | YC | | | | |
| ADI-81819 | IGKV1-33 | EIVMTQ | 1891 | QASQDI | 3841 | WYQQKP | 2257 | DASNLE | 2416 | GVPSRF | 4274 | QQYYNL | 4467 | FGGGTK |
| | | SPSSLS | | FNYLT | | GKAPKL | | T | | SGSGSG | | PPT | | VEIK |
| | | ASVGDR | | | | LIY | | | | TDFTLT | | | | |
| | | VTITC | | | | | | | | ISSLQP | | | | |
| | | | | | | | | | | EDIATY | | | | |
| | | | | | | | | | | SC | | | | |
| ADI-81621 | IGLV3-21 | QHVLTQ | 3640 | GVNNFG | 3842 | WYQQKP | 2283 | DDSDRP | 2399 | GIPERF | 2660 | QVWDST | 4468 | FGGGTK |
| | | PPSVSV | | SKSVP | | GQAPVL | | S | | SGSNSG | | SDHWV | | LTVL |
| | | APGQTA | | | | VVY | | | | NTATLT | | | | |
| | | SITC | | | | | | | | ISRVEV | | | | |
| | | | | | | | | | | GDEADY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81626 | 2937 | GACATCCCGATGACCCAGTCTCCAGACTCCCTGGCTGTGTCTC | 4781 | DIPMTQSPDSLAVSL | 5135 | 5 | 5 |
| | | TGGGCGAGAGGGCCACCATCAACTGCAAGACCAGCCAGAGTGT | | GERATINCKTSQSVL | | | |
| | | TTTAGACAGGTCCGACAATAGGAACTACTTAGCTTGGTACCAG | | DRSDNRNYLAWYQQK | | | |
| | | CAGAAACCAGGACAGCCTCCTAAGCTGCTCATTTACTGGGCAT | | PGQPPKLLIYWASTR | | | |
| | | CTACGCGGGAATCCGGGGTCCCTGACCGATTCAGTGGCAGCGG | | ESGVPDRFSGSGSGT | | | |
| | | GTCTGGGACAGATTTCACTCTCACCATCAGCAGCCTGCAGGCT | | DFTLTISSLQAEDVA | | | |
| | | GAAGATGTGGCAGTTTATTACTGTCAACAATGTTCTGTTACTC | | VYYCQQCSVTPPMCS | | | |
| | | CTCCGATGTGCAGTTTTGGCCAGGGGACCAAGGTGGAAATCAA | | FGQGTKVEIK | | | |
| | | A | | | | | |
| ADI-81803 | 2937 | GACATCCAGATGACCCAGTCTCCATCCTCCCTGTCTGCATCTG | 4782 | DIQMTQSPSSLSASV | 5136 | 5 | 5 |
| | | TAGGAGACAGAGTCACCATCACTTGCCGGGCAAGTCAGAACAT | | GDRVTITCRASQNIN | | | |
| | | TAACACCTATTTAAATTGGTATCAGCAGAAACCAGGGAAAGCC | | TYLNWYQQKPGKAPK | | | |
| | | CCTAAGGTCCTGATCTATGCTGCATCCAGTTTGCAAAGTGGGG | | VLIYAASSLQSGVPS | | | |
| | | TCCCGTCAAGGTTCACTGGCAGTGGATCTGGGACAGATTTCAC | | RFTGSGSGTDFTLTI | | | |
| | | TCTCACCATCAGCAGTCTGCAACCTGAAGATTTTGCAACTTAC | | SSLQPEDFATYYCQQ | | | |
| | | TACTGTCAACAGAGTTACAGTTCCCCTCGGACGTTCGGCCAAG | | SYSSPRTFGQGTKVE | | | |
| | | GGACCAAGGTGGAAATCAAA | | IK | | | |
| ADI-81882 | 2942 | GACATCACACTGACGCAGTCTCCATCCTCCCTGTCTGCATCTG | 4783 | DITLTQSPSSLSASV | 5137 | 5 | 5 |
| | | TAGGAGACAGAGTCACCTTCACTTGCCAGGCGAGTCAGGACAT | | GDRVTFTCQASQDIN | | | |
| | | TAACAACTTTTTAAATTGGTATCAACAAAAACCAGGGAAAGCC | | NFLNWYQQKPGKAPK | | | |
| | | CCTAAACTCCTGATCTTCGATGTATCGAATTTGGAAACAGGGG | | LLIFDVSNLETGVPS | | | |
| | | TCCCATCAAGATTCAGTGGCAGTGGATCTGGGACAGATTTTAC | | RFSGSGSGTDFTFTI | | | |
| | | TTTCACCATCAGCAGCCTGCAGCCTGAAGATATTGCAACATAT | | SSLQPEDIATYYCQH | | | |
| | | TACTGTCAACACTATGATAATCTCAAATTCACTTTCGGCCCTG | | YDNLKFTFGPGTKVE | | | |
| | | GGACCAAGGTGGAAATCAAA | | IK | | | |
| ADI-81911 | 2938 | GAAATTGTGATGACCCAGTCTCCATCCTCCCTGTCTGCATCTG | 4784 | EIVMTQSPSSLSASV | 5138 | 5 | 4 |
| | | TAGGAGACAGAGTCACCATCACTTGCCGGGCAAGTCAGAGCAT | | GDRVTITCRASQSIY | | | |
| | | TTACAACTACTTAAATTGGTATCAGCAGAAACCAGGGAAAGCC | | NYLNWYQQKPGKAPK | | | |
| | | CCTAAGCTCCTGATCTATGGTGCATCCAGTTTGCAAAGTGGGG | | LLIYGASSLQSGVPS | | | |
| | | TCCCATCAAGGATCAGTGGCAGTGGATCTGGGACAGATTTCAC | | RISGSGSGTDFTLTI | | | |
| | | TCTCACCATCAGCAGTCTGCAACCTGAAGATTTTGCAACTTAC | | SSLQPEDFATYYCQQ | | | |
| | | TACTGTCAACAGAGTTACAGTACCCCGGGCACTTTCGGCGGAG | | SYSTPGTFGGGTKVD | | | |
| | | GGACCAAGGTGGATATCAAA | | IK | | | |
| ADI-81557 | 2937 | GAAATTGTGTTGACCCAGTCTCCAGGCACCCTGTCTTTGTCTC | 4785 | EIVLTQSPGTLSLSP | 5139 | 5 | 5 |
| | | CAGGGGAAAGAGCCACCCTCTCCTGCAGGGCCAGTCAGAGTGT | | GERATLSCRASQSVS | | | |
| | | TAGCAGCAACTACTTAGCCTGGTACCAGCAGAAACCTGGCCAG | | SNYLAWYQQKPGQAP | | | |
| | | GCTCCCAGGCTCCTCATCTCTGATGCATCCAGCAGGGCCGCTG | | RLLISDASSRAAGIP | | | |
| | | GCATCCCAGACAGGTTCAGTGGCAGTGGGTCTGGGACAGACTT | | DRFSGSGSGTDFTLT | | | |
| | | CACTCTCACCATCGGCAGACTGGAGCCTGAAGATTTTGCAGTG | | IGRLEPEDFAVYYCQ | | | |
| | | TATTACTGTCAGCAGTATGGTAGCTCACCTGGCAGTTTTGGCC | | QYGSSPGSFGQGTKV | | | |
| | | AGGGGACCAAGGTGGAAATCAAA | | EIK | | | |
| ADI-81886 | 2937 | GACATGACACTCACGCAGTCTCCAGACTCCCTGGCTGTGTCTC | 4786 | DMTLTQSPDSLAVSL | 5140 | 5 | 5 |
| | | TGGGCGAGAGGGCCACCATCAACTGCAAGTCCAGCCAGAGTGT | | GERATINCKSSQSVL | | | |
| | | TTTATCCAGCTCCAACAATAAGAACTACTTAACTTGGTACCAG | | SSSNNKNYLTWYQQK | | | |
| | | CAAAAACCGGGACGGCCTCCTAAGCTGCTCATTTACTGGGCTT | | PGRPPKLLIYWASTR | | | |
| | | CCACCCGGGAATCCGGGGTCCCTGACCGATTCAGTGGCGGCGG | | ESGVPDRFSGGGSGT | | | |
| | | GTCTGGGACAGATTTCACTCTCTCCATCAGCAGTCTGCAGGCT | | DFTLSISSLQAEDVA | | | |
| | | GAGGATGTGGCAGTTTATTACTGTCAGCAATCTTATAGTCCTC | | VYYCQQSYSPPYTFG | | | |
| | | CGTACACTTTTGGCCAGGGGACCAAGGTGGAAATCAAA | | QGTKVEIK | | | |
| ADI-81819 | 2936 | GAAATTGTGATGACCCAGTCTCCATCCTCCCTGTCTGCATCTG | 4787 | EIVMTQSPSSLSASV | 5141 | 5 | 4 |
| | | TAGGAGACAGAGTCACCATCACTTGCCAGGCGAGTCAGGACAT | | GDRVTITCQASQDIF | | | |
| | | TTTCAACTATTTAACTTGGTATCAACAGAAGCCAGGGAAAGCC | | NYLTWYQQKPGKAPK | | | |
| | | CCTAAGCTCCTGATCTACGATGCATCCAATTTGGAAACAGGGG | | LLIYDASNLETGVPS | | | |
| | | TCCCATCAAGGTTCAGTGGAAGTGGATCTGGGACAGATTTTAC | | RFSGSGSGTDFTLTI | | | |
| | | TTTAACCATCAGCAGCCTGCAGCCTGAAGATATTGCAACATAT | | SSLQPEDIATYSCQQ | | | |
| | | TCCTGTCAACAGTATTATAATCTCCCTCCGACTTTCGGCGGAG | | YYNLPPTFGGGTKVE | | | |
| | | GGACCAAGGTGGAGATCAAA | | IK | | | |
| ADI-81621 | 2939 | CAGCATGTGCTGACTCAGCCACCCTCGGTGTCAGTGGCCCCAG | 4788 | EIVMTQSPSSLSASV | 5141 | 5 | 5 |
| | | GACAGACGGCCAGCATCACCTGTGGGGTAAACAACTTTGGAAG | | GDRVTITCQASQDIF | | | |
| | | TAAAAGTGTACCCTGGTACCAGCAGAAGCCAGGCCAGGCCCCT | | NYLTWYQQKPGKAPK | | | |
| | | GTCCTGGTCGTCTATGATGATAGCGACCGGCCCTCAGGGATCC | | LLIYDASNLETGVPS | | | |
| | | CTGAGCGATTCTCTGGCTCCAACTCTGGGAACACGGCCACCCT | | RFSGSGSGTDFTLTI | | | |
| | | GACCATCAGCAGGGTCGAAGTCGGGGATGAGGCCGACTATTAC | | SSLQPEDIATYSCQQ | | | |
| | | TGTCAGGTGTGGGATAGTACTAGTGATCATTGGGTGTTCGGCG | | YYNLPPTFGGGTKVE | | | |
| | | GAGGGACCAAGCTCACCGTCCTA | | IK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81821 | IGLV1-40 | QSVLTQ | 1948 | TGSNSN | 2143 | WYQQLP | 3998 | INSNRP | 4121 | GVPDRF | 2599 | QSYDSS | 2908 | FGGGTK |
| | | PPSVSG | | IGAGYD | | GTAPTL | | S | | SGSKSG | | LSGSV | | VTVL |
| | | APGQRV | | VH | | LIS | | | | TSASLA | | | | |
| | | TISC | | | | | | | | ITGLQA | | | | |
| | | | | | | | | | | EDEADY | | | | |
| | | | | | | | | | | YC | | | | |
| ADI-81857 | IGKV1-12 | DIPLTQ | 3641 | RASQDI | 3843 | WYQQKP | 2294 | AATTLQ | 4122 | GVPSRF | 2534 | QQANTF | 4469 | FGQGTK |
| | | SPSSVS | | SGWLA | | GKAPRL | | S | | SGSGSG | | PRT | | VDIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81909 | IGKV1D-39 | DIPVTQ | 3642 | RASQSI | 2090 | WYQHKP | 3924 | AVSSLQ | 4123 | GVPSRF | 4275 | QQNYNS | 4470 | FGGGTK |
| | | SPSSLS | | SSFLN | | GKAPKL | | S | | SGSEYG | | PLT | | VEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81564 | IGLV1-40 | QSVLTQ | 1948 | TGSNSN | 2143 | WYQQLP | 2314 | DNTNRP | 2450 | GVPDRF | 4276 | QSYDSS | 2872 | FGGGTQ |
| | | PPSVSG | | IGAGYD | | GTAPKL | | S | | SGSKSG | | LIGSL | | LTVL |
| | | APGQRV | | VH | | LIY | | | | TSASLT | | | | |
| | | TISC | | | | | | | | ITGLQA | | | | |
| | | | | | | | | | | EDEADY | | | | |
| | | | | | | | | | | YC | | | | |
| ADI-81606 | IGKV1D-39 | DIQMTQ | 1878 | RASQSI | 2134 | WYQQKP | 2257 | AAFILQ | 4124 | GVPSRF | 2534 | QQSYSA | 2775 | FGRGTK |
| | | SPSSLS | | SRYLN | | GKAPKL | | S | | SGSGSG | | PPWT | | VDIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81782 | IGKV1-NL1 | DIRVTQ | 3618 | RASQAI | 2051 | WYQQKP | 2259 | AASTLE | 2398 | GVPPRF | 4277 | QQYYTF | 4471 | FGQGTK |
| | | SPSSLS | | SNSLA | | GKAPKL | | S | | SGSGSG | | RT | | VDIK |
| | | ASVGDR | | | | LLY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81627 | IGKV3-11 | EIVMTQ | 1996 | RASQSV | 3844 | WYQQKP | 2258 | DTSNRA | 4125 | GIPARF | 2607 | QLRNNW | 4472 | FGGGTK |
| | | SPATLS | | TNYLA | | GQAPRL | | T | | SGSGSG | | PPGALT | | VEIK |
| | | LSPGER | | | | LIY | | | | TDFTLT | | | | |
| | | ATLSC | | | | | | | | ISSLEP | | | | |
| | | | | | | | | | | EDFAVY | | | | |
| | | | | | | | | | | YC | | | | |
| ADI-81609 | IGKV3D-15 | DITMTQ | 3643 | RASQSV | 3845 | WYQQKP | 2258 | GATTRA | 4126 | GIPARF | 4278 | QQYNNW | 4473 | FGGGTK |
| | | TPATLS | | SGNLA | | GQAPRL | | S | | SGSESG | | LT | | VEIK |
| | | VSPGER | | | | LIY | | | | TEFTLT | | | | |
| | | ATLSC | | | | | | | | ISSLQS | | | | |
| | | | | | | | | | | EDFAVY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81821 | 2947 | CAGTCTGTCTTGACGCAGCCGCCCTCAGTGTCTGGGGCCCCAG | 4789 | QSVLTQPPSVSGAPG | 5142 | 5 | 4 |
| | | GGCAGAGGGTCACCATCTCCTGCACTGGGAGCAACTCCAACAT | | QRVTISCTGSNSNIG | | | |
| | | CGGGGCAGGTTATGATGTACACTGGTACCAGCAGCTTCCAGGA | | AGYDVHWYQQLPGTA | | | |
| | | ACAGCCCCCACACTCCTCATCTCTATTAACAGCAATCGGCCCT | | PTLLISINSNRPSGV | | | |
| | | CAGGGGTCCCTGACCGATTCTCTGGCTCCAAGTCTGGCACCTC | | PDRFSGSKSGTSASL | | | |
| | | AGCCTCCCTGGCCATCACTGGGCTCCAGGCTGAGGATGAGGCT | | AITGLQAEDEADYYC | | | |
| | | GATTATTACTGCCAGTCCTATGACAGCAGCCTGAGTGGTTCGG | | QSYDSSLSGSVFGGG | | | |
| | | TATTCGGCGGAGGGACCAAGGTCACCGTCCTA | | TKVTVL | | | |
| ADI-81857 | 2944 | GACATCCCACTCACCCAGTCTCCATCTTCCGTGTCTGCATCTG | 4790 | DIPLTQSPSSVSASV | 5143 | 5 | 5 |
| | | TGGGAGACAGAGTCACCATCACTTGTCGGGCGAGTCAGGATAT | | GDRVTITCRASQDIS | | | |
| | | TAGCGGCTGGTTAGCCTGGTATCAGCAGAAACCAGGGAAAGCC | | GWLAWYQQKPGKAPR | | | |
| | | CCTAGGCTCCTGATCTATGCTGCAACCACTTTGCAAAGTGGGG | | LLIYAATTLQSGVPS | | | |
| | | TCCCATCAAGGTTCAGCGGCAGTGGCTCTGGGACAGATTTCAC | | RFSGSGSGTDFTLTI | | | |
| | | TCTCACCATCAGCAGCCTGCAGCCTGAAGATTTTGCAACTTAC | | SSLQPEDFATYYCQQ | | | |
| | | TATTGTCAACAGGCTAACACTTTCCCCCGGACGTTCGGCCAAG | | ANTFPRTFGQGTKVD | | | |
| | | GGACCAAGGTGGATATCAAA | | IK | | | |
| ADI-81909 | 2936 | GACATCCCGGTGACCCAGTCTCCATCCTCCCTGTCTGCATCTG | 4791 | DIPVTQSPSSLSASV | 5144 | 5 | 5 |
| | | TAGGAGACAGAGTCACCATCACTTGCCGGGCAAGTCAGAGCAT | | GDRVTITCRASQSIS | | | |
| | | TAGCAGCTTTTTAAATTGGTATCAACACAAACCAGGGAAAGCC | | SFLNWYQHKPGKAPK | | | |
| | | CCTAAGCTCCTGATCTATGCTGTATCCAGTTTGCAAAGTGGGG | | LLIYAVSSLQSGVPS | | | |
| | | TCCCATCAAGGTTCAGTGGCAGTGAATATGGGACAGATTTCAC | | RFSGSEYGTDFTLTI | | | |
| | | TCTCACCATCAGCAGTTTGCAACCTGAAGATTTTGCAACTTAC | | SSLQPEDFATYYCQQ | | | |
| | | TACTGTCAACAAAATTACAATTCCCCCCTCACTTTCGGCGGAG | | NYNSPLTFGGGTKVE | | | |
| | | GGACCAAGGTGGAGATCAAA | | IK | | | |
| ADI-81564 | 2948 | CAGTCTGTGTTGACGCAGCCGCCCTCAGTGTCTGGGGCCCCAG | 4792 | QSVLTQPPSVSGAPG | 5145 | 4 | 4 |
| | | GGCAGAGGGTCACCATCTCCTGCACTGGGAGCAACTCCAACAT | | QRVTISCTGSNSNIG | | | |
| | | CGGGGCAGGTTATGATGTACACTGGTACCAGCAACTTCCAGGA | | AGYDVHWYQQLPGTA | | | |
| | | ACAGCCCCCAAACTCCTCATCTATGATAACACCAATCGGCCCT | | PKLLIYDNTNRPSGV | | | |
| | | CAGGGGTCCCTGACCGATTCTCTGGCTCCAAGTCTGGCACCTC | | PDRFSGSKSGTSASL | | | |
| | | AGCCTCCCTGACCATCACTGGGCTCCAGGCTGAGGATGAGGCT | | TITGLQAEDEADYYC | | | |
| | | GATTACTACTGCCAGTCCTATGACAGCAGCCTGATTGGTTCGC | | QSYDSSLIGSLFGGG | | | |
| | | TGTTCGGCGGAGGCACCCAGCTGACCGTCCTC | | TQLTVL | | | |
| ADI-81606 | 4599 | GACATCCAGATGACCCAGTCTCCATCCTCCCTGTCTGCGTCTG | 4793 | DIQMTQSPSSLSASV | 5146 | 4 | 3 |
| | | TTGGCGACAGAGTCACCATCACTTGCCGGGCAAGTCAGAGCAT | | GDRVTITCRASQSIS | | | |
| | | TAGCAGATATTTAAATTGGTATCAGCAGAAACCAGGGAAAGCC | | RYLNWYQQKPGKAPK | | | |
| | | CCTAAGCTCCTGATCTATGCTGCATTTATTTTGCAAAGTGGGG | | LLIYAAFILQSGVPS | | | |
| | | TCCCATCAAGGTTCAGTGGCAGTGGATCTGGAACAGATTTCAC | | RFSGSGSGTDFTLTI | | | |
| | | TCTCACCATCAGCAGTCTGCAACCTGAAGATTTTGCAACTTAT | | SSLQPEDFATYYCQQ | | | |
| | | TACTGTCAACAGAGTTACAGTGCCCCTCCGTGGACGTTCGGCC | | SYSAPPWTFGRGTKV | | | |
| | | GAGGGACCAAGGTGGATATCAAA | | DIK | | | |
| ADI-81782 | 2944 | GACATCCGGGTGACCCAGTCTCCATCCTCCCTGTCTGCATCTG | 4794 | DIRVTQSPSSLSASV | 5147 | 4 | 4 |
| | | TAGGAGACAGAGTCACCATCACTTGCCGGGCGAGTCAGGCCAT | | GDRVTITCRASQAIS | | | |
| | | TAGCAATTCTTTAGCCTGGTATCAGCAGAAACCAGGTAAAGCC | | NSLAWYQQKPGKAPK | | | |
| | | CCGAAACTCCTTCTCTATGCTGCATCCACATTGGAAAGTGGGG | | LLLYAASTLESGVPP | | | |
| | | TCCCACCCAGGTTCAGTGGCAGTGGGTCTGGGACGGATTTCAC | | RFSGSGSGTDFTLTI | | | |
| | | TCTCACCATCAGCAGCCTGCAGCCTGAAGATTTTGCAACTTAT | | SSLQPEDFATYYCQQ | | | |
| | | TACTGTCAACAGTACTATACTTTCCGGACGTTCGGCCAAGGGA | | YYTFRTFGQGTKVDI | | | |
| | | CCAAAGTGGATATCAAA | | K | | | |
| ADI-81627 | 2936 | GAAATTGTGATGACCCAGTCTCCAGCCACCCTGTCTTTGTCTC | 4795 | EIVMTQSPATLSLSP | 5148 | 4 | 4 |
| | | CAGGGGAAAGAGCCACCCTCTCCTGCAGGGCCAGTCAGAGTGT | | GERATLSCRASQSVT | | | |
| | | TACCAACTACTTAGCCTGGTACCAACAGAAACCTGGCCAGGCT | | NYLAWYQQKPGQAPR | | | |
| | | CCCAGGCTCCTCATCTATGATACATCCAACAGGGCCATTGGCA | | LLIYDTSNRAIGIPA | | | |
| | | TCCCAGCCAGGTTCAGTGGCAGTGGGTCTGGGACAGACTTCAC | | RFSGSGSGTDFTLTI | | | |
| | | TCTCACCATCAGCAGCCTAGAGCCTGAAGATTTTGCAGTTTAT | | SSLEPEDFAVYYCQL | | | |
| | | TACTGTCAACTACGTAACAACTGGCCTCCGGGAGCGCTCACTT | | RNNWPPGALTFGGGT | | | |
| | | TCGGCGGAGGGACCAAGGTGGAAATCAAA | | KVEIK | | | |
| ADI-81609 | 2936 | GACATCACAATGACGCAGACTCCAGCCACCCTGTCTGTGTCTC | 4796 | DITMTQTPATLSVSP | 5149 | 4 | 4 |
| | | CAGGGGAAAGAGCCACCCTCTCCTGCAGGGCCAGTCAGAGTGT | | GERATLSCRASQSVS | | | |
| | | TAGCGGCAACTTAGCCTGGTACCAGCAGAAACCTGGCCAGGCT | | GNLAWYQQKPGQAPR | | | |
| | | CCCAGGCTCCTCATCTATGGTGCAACCACCAGGGCCTCTGGTA | | LLIYGATTRASGIPA | | | |
| | | TCCCAGCCAGGTTCAGTGGCAGTGAGTCTGGGACAGAGTTCAC | | RFSGSESGTEFTLTI | | | |
| | | TCTCACCATCAGCAGCCTACAGTCTGAAGATTTTGCAGTTTAT | | SSLQSEDFAVYYCQQ | | | |
| | | TACTGTCAACAATATAATAACTGGCTCACTTTCGGCGGAGGGA | | YNNWLTFGGGTKVEI | | | |
| | | CCAAGGTGGAAATCAAA | | K | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81777 | IGKV3-20 | ETALTQ | 3644 | RASESV | 3846 | WYQQKP | 3912 | GAASRA | 4127 | GIPDRF | 2616 | QQYHNS | 4474 | FGQGTK |
| | | SPGTLS | | SSGYLA | | GQAPRL | | T | | SGSGSG | | PRT | | VEIK |
| | | LSPGER | | | | LIF | | | | TDFTLT | | | | |
| | | ATLSC | | | | | | | | ISRLEP | | | | |
| | | | | | | | | | | EDFAVY | | | | |
| | | | | | | | | | | YC | | | | |
| ADI-81728 | IGKV1D-39 | EIGMTQ | 3552 | RASQSI | 2050 | WYQQKP | 2257 | TASSLQ | 2506 | GVPSRF | 4279 | QQSYST | 2905 | FGQGTK |
| | | SPSSLS | | SSYLN | | GKAPKL | | S | | SGSGCG | | PLT | | VEIK |
| | | ASVGDR | | | | LIY | | | | TDFTLT | | | | |
| | | VTITC | | | | | | | | ISSVQR | | | | |
| | | | | | | | | | | EDFATY | | | | |
| | | | | | | | | | | YC | | | | |
| ADI-81732 | IGKV1D-39 | EMTLTQ | 1914 | RASQTI | 3847 | WYQQKP | 2257 | AASTLQ | 2392 | GVPSRF | 2534 | QQSYVL | 4475 | FGQGTK |
| | | SPSSLS | | NRYLN | | GKAPKL | | S | | SGSGSG | | PMYT | | VEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81588 | IGKV1D-39 | DIVLTQ | 3645 | RASQSI | 2050 | WYQQKP | 2257 | GASSLH | 2407 | GVPSRF | 2534 | QQSYST | 2729 | FGQGTK |
| | | SPSSLP | | SSYLN | | GKAPKL | | T | | SGSGSG | | PRT | | VDIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81685 | IGKV1D-39 | EIVLTQ | 1888 | RASQSI | 2249 | WYQQKP | 2257 | TASSLQ | 2506 | GVPSRF | 2534 | QQSYNT | 2702 | FGQGTK |
| | | SPSSLS | | GTYLN | | GKAPKL | | S | | SGSGSG | | PPWT | | VEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81589 | IGKV1D-39 | DIQMTQ | 1878 | RASQTI | 3848 | WYQQKP | 2257 | AGSTLQ | 4128 | GVPSRF | 2534 | QQSDTT | 4476 | FGGGTK |
| | | SPSSLS | | STYLN | | GKAPKL | | S | | SGSGSG | | PQIT | | VEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81592 | IGKV1-5 | DITLTQ | 3646 | RASQSI | 3849 | WYQQKP | 3999 | DASSLE | 2434 | GVPSRF | 2532 | QQYHSF | 4477 | FGGGTK |
| | | SPSTLS | | NTWLA | | GNAPKL | | S | | SGSGSG | | SPLT | | VEIK |
| | | ASVGAR | | | | LIY | | | | TEFTLT | | | | |
| | | VTITC | | | | | | | | ISSLQP | | | | |
| | | | | | | | | | | DDFATY | | | | |
| | | | | | | | | | | YC | | | | |
| ADI-81751 | IGKV2D-28 | EIVMTQ | 1923 | RYTQSL | 3850 | WYLQKP | 2260 | LGSHRA | 4129 | GVPDRF | 2533 | MQALQT | 4478 | FGQGTK |
| | | SPLSLP | | LLSNGY | | GQSPQL | | S | | SGSGSG | | PQT | | VDIK |
| | | VTPGEP | | NYLD | | LIY | | | | TDFTLK | | | | |
| | | ASISC | | | | | | | | ISRVEA | | | | |
| | | | | | | | | | | EDVGVY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81777 | 2937 | GAAACGGCACTGACGCAGTCTCCAGGCACCCTGTCTTTGTCTC | 4797 | ETALTQSPGTLSLSP | 5150 | 4 | 4 |
| | | CGGGGGAAAGAGCCACCCTCTCCTGCAGGGCCAGTGAGAGTGT | | GERATLSCRASESVS | | | |
| | | TAGCAGCGGCTACTTAGCCTGGTACCAGCAGAAACCTGGCCAG | | SGYLAWYQQKPGQAP | | | |
| | | GCTCCCAGGCTCCTCATCTTTGGTGCCGCCAGCAGGGCCACTG | | RLLIFGAASRATGIP | | | |
| | | GCATCCCAGACAGGTTCAGTGGCAGTGGGTCTGGGACAGACTT | | DRFSGSGSGTDFTLT | | | |
| | | CACTCTCACCATCAGCAGACTGGAGCCTGAAGATTTTGCAGTA | | ISRLEPEDFAVYYCQ | | | |
| | | TATTACTGTCAGCAGTATCATAACTCACCTCGGACGTTCGGCC | | QYHNSPRTFGQGTKV | | | |
| | | AAGGGACCAAGGTGGAAATCAAA | | EIK | | | |
| ADI-81728 | 2937 | GAAATCGGAATGACCCAGTCTCCATCCTCCCTGTCTGCATCTG | 4798 | EIGMTQSPSSLSASV | 5151 | 4 | 4 |
| | | TAGGAGACAGAGTCACCATCACTTGCCGGGCAAGTCAGAGCAT | | GDRVTITCRASQSIS | | | |
| | | TAGCAGCTATTTAAATTGGTATCAGCAGAAACCAGGGAAAGCC | | SYLNWYQQKPGKAPK | | | |
| | | CCTAAACTTTTAATCTATACTGCATCCAGTTTGCAAAGTGGGG | | LLIYTASSLQSGVPS | | | |
| | | TCCCATCAAGGTTCAGTGGCAGTGGATGTGGGACAGATTTCAC | | RFSGSGCGTDFTLTI | | | |
| | | TCTCACCATCAGCAGTGTGCAACGTGAAGATTTTGCTACTTAC | | SSVQREDFATYYCQQ | | | |
| | | TACTGTCAACAGAGTTACAGTACCCCTCTAACGTTCGGCCAAG | | SYSTPLTFGQGTKVE | | | |
| | | GGACCAAGGTGGAAATCAAA | | IK | | | |
| ADI-81732 | 2937 | GAAATGACACTCACGCAGTCTCCATCCTCCCTGTCTGCATCTG | 4799 | EMTLTQSPSSLSASV | 5152 | 4 | 4 |
| | | TTGGAGACAGAGTCACCATCACTTGCCGGGCAAGTCAGACCAT | | GDRVTITCRASQTIN | | | |
| | | TAACAGATATTTAAATTGGTATCAGCAGAAACCAGGGAAAGCC | | RYLNWYQQKPGKAPK | | | |
| | | CCTAAGCTCCTGATCTATGCTGCATCCACTTTGCAAAGTGGGG | | LLIYAASTLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTCCAACCTGAAGATTTTGCAACTTAC | | SSLQPEDFATYYCQQ | | | |
| | | TACTGTCAACAGAGTTATGTTCTCCCCATGTACACTTTTGGCC | | SYVLPMYTFGQGTKV | | | |
| | | AGGGGACCAAGGTGGAGATCAAA | | EIK | | | |
| ADI-81588 | 2944 | GACATCGTGTTGACCCAGTCTCCATCCTCCCTGCCTGCATCTG | 4800 | DIVLTQSPSSLPASV | 5153 | 4 | 4 |
| | | TAGGAGACAGAGTCACCATCACTTGCCGGGCAAGTCAGAGCAT | | GDRVTITCRASQSIS | | | |
| | | TAGCAGTTATTTAAATTGGTATCAGCAAAAACCAGGGAAAGCC | | SYLNWYQQKPGKAPK | | | |
| | | CCTAAGCTCCTGATTTATGGTGCATCCAGTTTGCATACTGGGG | | LLIYGASSLHTGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAACCTGAAGATTTTGCAACTTAC | | SSLQPEDFATYYCQQ | | | |
| | | TACTGTCAACAGAGTTACAGTACCCCTCGGACTTTTGGCCAGG | | SYSTPRTFGQGTKVD | | | |
| | | GGACCAAGGTGGATATCAAA | | IK | | | |
| ADI-81685 | 2937 | GAAATTGTGTTGACGCAGTCTCCATCCTCCCTGTCTGCATCTG | 4801 | EIVLTQSPSSLSASV | 5154 | 4 | 3 |
| | | TAGGAGACAGAGTCACCATCACTTGCCGGGCAAGTCAGAGCAT | | GDRVTITCRASQSIG | | | |
| | | TGGGACCTATTTAAATTGGTATCAGCAGAAACCGGGGAAAGCC | | TYLNWYQQKPGKAPK | | | |
| | | CCTAAGCTCCTGATCTATACTGCATCCAGTTTGCAAAGTGGGG | | LLIYTASSLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAACCTGAAGATTTTGCAACTTAC | | SSLQPEDFATYYCQQ | | | |
| | | TACTGTCAACAGAGTTACAATACCCCTCCGTGGACGTTCGGCC | | SYNTPPWTFGQGTKV | | | |
| | | AAGGGACCAAGGTGGAGATCAAA | | EIK | | | |
| ADI-81589 | 2936 | GACATCCAGATGACCCAGTCTCCATCCTCCCTGTCTGCATCTG | 4802 | DIQMTQSPSSLSASV | 5155 | 4 | 4 |
| | | TAGGAGACAGAGTCACCATTACTTGCCGGGCAAGTCAGACCAT | | GDRVTITCRASQTIS | | | |
| | | TAGCACCTATTTAAATTGGTATCAGCAGAAACCAGGGAAAGCC | | TYLNWYQQKPGKAPK | | | |
| | | CCTAAGCTCCTGATCTATGCTGGATCCACTTTGCAAAGTGGGG | | LLIYAGSTLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAACCTGAGGATTTTGCAACTTAC | | SSLQPEDFATYYCQQ | | | |
| | | TACTGTCAACAGAGTGACACTACCCCTCAGATCACTTTCGGCG | | SDTTPQITFGGGTKV | | | |
| | | GAGGGACCAAGGTGGAAATCAAA | | EIK | | | |
| ADI-81592 | 2936 | GACATCACATTGACGCAGTCTCCTTCCACCCTGTCTGCATCTG | 4803 | DITLTQSPSTLSASV | 5156 | 4 | 4 |
| | | TAGGAGCCAGAGTCACCATCACTTGCCGGGCCAGTCAGAGTAT | | GARVTITCRASQSIN | | | |
| | | TAATACCTGGTTGGCCTGGTATCAGCAGAAACCAGGGAACGCC | | TWLAWYQQKPGNAPK | | | |
| | | CCTAAACTCCTGATCTATGATGCCTCCTCTTTGGAAAGTGGGG | | LLIYDASSLESGVPS | | | |
| | | TCCCTTCAAGATTCAGCGGCAGTGGATCTGGGACAGAATTCAC | | RFSGSGSGTEFTLTI | | | |
| | | TCTCACCATCAGCAGCCTGCAGCCTGATGATTTTGCAACTTAT | | SSLQPDDFATYYCQQ | | | |
| | | TACTGCCAACAGTATCATAGTTTTTCTCCGCTCACTTTCGGCG | | YHSFSPLTFGGGTKV | | | |
| | | GAGGGACCAAGGTGGAAATCAAA | | EIK | | | |
| ADI-81751 | 2944 | GAAATTGTGATGACGCAGTCTCCACTCTCCCTGCCCGTCACCC | 4804 | EIVMTQSPLSLPVTP | 5157 | 4 | 4 |
| | | CTGGAGAGCCGGCCTCCATCTCCTGCAGGTATACTCAGAGCCT | | GEPASISCRYTQSLL | | | |
| | | CCTGCTTAGTAATGGATACAACTACTTGGATTGGTACCTGCAG | | LSNGYNYLDWYLQKP | | | |
| | | AAGCCAGGGCAGTCTCCACAACTCCTGATCTATTTGGGTTCTC | | GQSPQLLIYLGSHRA | | | |
| | | ATCGGGCCTCCGGGGTCCCTGACAGGTTCAGTGGCAGTGGATC | | SGVPDRFSGSGSGTD | | | |
| | | AGGCACAGATTTTACACTGAAAATCAGCAGAGTGGAGGCTGAG | | FTLKISRVEAEDVGV | | | |
| | | GATGTTGGGGTTTATTACTGCATGCAAGCTCTACAAACTCCTC | | YYCMQALQTPQTFGQ | | | |
| | | AGACGTTCGGCCAAGGGACCAAAGTGGATATCAAA | | GTKVDIK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81533 | IGKV1D-39 | DIQMTQ | 1897 | RASQSI | 2066 | WYQQKP | 2263 | AASSLQ | 2391 | GVPSRF | 4280 | QQSYNI | 4440 | FGGGTK |
| | | SPSSLS | | STYLN | | GKAPNL | | S | | SGSGSG | | PQLT | | VEIK |
| | | ASVGDR | | | | LIY | | | | TDFTLT | | | | |
| | | ITITC | | | | | | | | ISSLQP | | | | |
| | | | | | | | | | | EDFAIY | | | | |
| | | | | | | | | | | YC | | | | |
| ADI-81548 | IGLV1-40 | QPVLTQ | 3647 | TGSNSN | 2143 | WYQQLP | 4000 | VNSNRP | 4113 | GVPDRF | 2622 | QSYDSS | 2853 | FGGGTK |
| | | PPSVSG | | IGAGYD | | GTAPKL | | S | | SGSKSD | | LSGVV | | VTVL |
| | | APGQRV | | VH | | LIF | | | | TSASLA | | | | |
| | | TISC | | | | | | | | ITGLQA | | | | |
| | | | | | | | | | | EDEADY | | | | |
| | | | | | | | | | | YC | | | | |
| ADI-81582 | IGLV1-40 | QSVVTQ | 1972 | TGSNSN | 2143 | WYQQLP | 2314 | DNNNRP | 2476 | GVPDRF | 2622 | QSYDSS | 2864 | FGTGTK |
| | | PPSVSG | | IGAGYD | | GTAPKL | | S | | SGSKSD | | LIGSV | | LTVL |
| | | APGQRV | | VH | | LIY | | | | TSASLA | | | | |
| | | TISC | | | | | | | | ITGLQA | | | | |
| | | | | | | | | | | EDEADY | | | | |
| | | | | | | | | | | YC | | | | |
| ADI-81541 | IGLV1-40 | QSVVTQ | 1972 | TGSNSN | 2143 | WYQQLP | 3992 | VNNNRP | 2471 | GVPDRF | 2599 | QSYDSS | 2908 | FGGGTK |
| | | PPSVSG | | IGAGYD | | GTAPKL | | S | | SGSKSG | | LSGSV | | LTVL |
| | | APGQRV | | VH | | LIS | | | | TSASLA | | | | |
| | | TISC | | | | | | | | ITGLQA | | | | |
| | | | | | | | | | | EDEADY | | | | |
| | | | | | | | | | | YC | | | | |
| ADI-81629 | IGLV1-40 | QSVLTQ | 1948 | TGSNSN | 2143 | WYQQLP | 2314 | INTNRP | 4103 | GVPDRF | 2599 | QSYDSS | 2908 | FGGGTK |
| | | PPSVSG | | IGAGYD | | GTAPKL | | S | | SGSKSG | | LSGSV | | LTVL |
| | | APGQRV | | VH | | LIY | | | | TSASLA | | | | |
| | | TISC | | | | | | | | ITGLQA | | | | |
| | | | | | | | | | | EDEADY | | | | |
| | | | | | | | | | | YC | | | | |
| ADI-81646 | IGKV1-9 | DMTMTQ | 3648 | RASQGI | 3851 | WYQQKP | 2325 | AASSLQ | 4130 | GVPSRF | 2592 | QQLSGY | 4479 | FGGGTK |
| | | SPSFLS | | SSYVA | | GKAPKL | | G | | SGSGSG | | FT | | VEIK |
| | | ASVGDR | | | | LMY | | | | TEFTLT | | | | |
| | | VTITC | | | | | | | | ISSLQP | | | | |
| | | | | | | | | | | EDFATY | | | | |
| | | | | | | | | | | YC | | | | |
| ADI-81745 | IGLV3-21 | SYVLTQ | 1883 | GEDGIG | 3852 | WYQQKP | 2283 | DDSDRP | 2399 | GIPERF | 2545 | QVWGFS | 4480 | FGGGTK |
| | | PPSVSV | | SKSVH | | GQAPVL | | S | | SGSNSG | | GDHWV | | LTVL |
| | | APGQTA | | | | VVY | | | | NTATLT | | | | |
| | | RITC | | | | | | | | ISRVEA | | | | |
| | | | | | | | | | | GDEADY | | | | |
| | | | | | | | | | | YC | | | | |
| ADI-81630 | IGLV1-40 | SSELSQ | 3649 | TGSNSN | 2143 | WYQQLP | 2314 | GNTNRP | 2484 | GVPDRF | 2622 | QSYDSG | 2903 | FGGGTK |
| | | QPSVSG | | IGAGYD | | GTAPKL | | S | | SGSKSD | | LTGSL | | LTVL |
| | | APGQRV | | VH | | LIY | | | | TSASLA | | | | |
| | | IISC | | | | | | | | ITGLQA | | | | |
| | | | | | | | | | | EDEADY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81533 | 2936 | GACATCCAGATGACCCAGTCTCCATCTTCCCTGTCTGCATCTG | 4805 | DIQMTQSPSSLSASV | 5158 | 4 | 4 |
| | | TAGGAGACAGAATCACCATCACTTGTCGGGCAAGTCAGAGCAT | | GDRITITCRASQSIS | | | |
| | | TAGCACCTATTTAAATTGGTATCAGCAGAAACCAGGGAAAGCC | | TYLNWYQQKPGKAPN | | | |
| | | CCTAACCTCCTGATCTATGCTGCATCCAGTTTGCAAAGTGGGG | | LLIYAASSLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGCCTGCAACCCGAAGATTTTGCAATTTAC | | SSLQPEDFAIYYCQQ | | | |
| | | TACTGTCAACAGAGTTACAATATTCCTCAACTCACTTTCGGCG | | SYNIPQLTFGGGTKV | | | |
| | | GAGGGACCAAGGTGGAAATCAAA | | EIK | | | |
| ADI-81548 | 2947 | CAGCCTGTGCTGACTCAGCCGCCCTCAGTGTCTGGGGCCCCAG | 4806 | QPVLTQPPSVSGAPG | 5159 | 4 | 4 |
| | | GGCAGAGGGTCACCATCTCCTGCACTGGGAGCAACTCCAACAT | | QRVTISCTGSNSNIG | | | |
| | | CGGGGCAGGTTATGATGTACACTGGTACCAGCAACTTCCAGGA | | AGYDVHWYQQLPGTA | | | |
| | | ACAGCCCCCAAACTCCTCATCTTTGTTAACAGCAATCGGCCCT | | PKLLIFVNSNRPSGV | | | |
| | | CAGGGGTCCCTGACCGATTCTCTGGCTCCAAGTCTGACACCTC | | PDRFSGSKSDTSASL | | | |
| | | AGCCTCCCTGGCCATCACTGGGCTCCAGGCTGAGGATGAGGCT | | AITGLQAEDEADYYC | | | |
| | | GATTATTACTGCCAGTCTTATGACAGCAGCCTGAGTGGTGTGG | | QSYDSSLSGVVFGGG | | | |
| | | TCTTCGGCGGAGGGACCAAGGTCACCGTCCTA | | TKVTVL | | | |
| ADI-81582 | 2949 | CAGTCTGTGGTGACGCAGCCGCCCTCAGTGTCTGGAGCCCCAG | 4807 | QSVVTQPPSVSGAPG | 5160 | 4 | 4 |
| | | GGCAGAGGGTCACCATCTCCTGCACTGGGAGCAACTCCAACAT | | QRVTISCTGSNSNIG | | | |
| | | CGGGGCAGGTTATGATGTACACTGGTACCAGCAGCTTCCAGGA | | AGYDVHWYQQLPGTA | | | |
| | | ACAGCCCCCAAACTCCTCATCTATGATAACAACAATCGCCCCT | | PKLLIYDNNNRPSGV | | | |
| | | CAGGGGTCCCTGACCGATTCTCTGGCTCCAAGTCTGACACCTC | | PDRFSGSKSDTSASL | | | |
| | | AGCCTCCCTGGCCATCACTGGGCTCCAGGCTGAGGATGAGGCT | | AITGLQAEDEADYYC | | | |
| | | GATTATTACTGCCAGTCCTATGATAGCAGCCTGATTGGTTCTG | | QSYDSSLIGSVFGTG | | | |
| | | TCTTCGGAACTGGGACCAAGCTCACCGTCCTA | | TKLTVL | | | |
| ADI-81541 | 2939 | CAGTCTGTGGTGACGCAGCCGCCCTCAGTGTCTGGGGCCCCAG | 4808 | QSVVTQPPSVSGAPG | 5161 | 4 | 4 |
| | | GGCAGAGAGTCACCATCTCCTGCACTGGGAGCAACTCCAACAT | | QRVTISCTGSNSNIG | | | |
| | | CGGGGCAGGCTATGATGTACACTGGTACCAGCAGCTTCCAGGA | | AGYDVHWYQQLPGTA | | | |
| | | ACAGCCCCCAAACTCCTCATCTCTGTTAACAACAATCGGCCCT | | PKLLISVNNNRPSGV | | | |
| | | CAGGGGTCCCTGACCGATTCTCTGGCTCCAAGTCTGGCACCTC | | PDRFSGSKSGTSASL | | | |
| | | AGCCTCCCTGGCCATCACTGGGCTCCAGGCTGAGGATGAGGCT | | AITGLQAEDEADYYC | | | |
| | | GATTATTACTGCCAGTCCTATGACAGCAGTCTGAGTGGTTCGG | | QSYDSSLSGSVFGGG | | | |
| | | TATTCGGCGGAGGGACCAAGCTCACCGTCCTA | | TKLTVL | | | |
| ADI-81629 | 2939 | CAGTCTGTTCTGACTCAGCCGCCCTCAGTGTCTGGGGCCCCAG | 4809 | QSVLTQPPSVSGAPG | 5162 | 4 | 3 |
| | | GGCAGAGGGTCACCATCTCCTGCACTGGGAGCAACTCCAACAT | | QRVTISCTGSNSNIG | | | |
| | | CGGGGCAGGTTATGATGTACACTGGTACCAGCAGCTTCCAGGA | | AGYDVHWYQQLPGTA | | | |
| | | ACAGCCCCCAAACTCCTCATCTATATTAACACCAATCGGCCCT | | PKLLIYINTNRPSGV | | | |
| | | CAGGGGTCCCTGACCGATTCTCTGGCTCCAAGTCTGGCACCTC | | PDRFSGSKSGTSASL | | | |
| | | AGCCTCCCTGGCCATCACTGGGCTCCAGGCTGAGGATGAGGCT | | AITGLQAEDEADYYC | | | |
| | | GATTATTACTGCCAGTCGTATGACAGCAGTCTGAGTGGTTCGG | | QSYDSSLSGSVFGGG | | | |
| | | TGTTCGGCGGAGGGACCAAGCTCACCGTCCTA | | TKLTVL | | | |
| ADI-81646 | 2936 | GACATGACGATGACGCAGTCTCCATCCTTCCTGTCTGCATCTG | 4810 | DMTMTQSPSFLSASV | 5163 | 4 | 4 |
| | | TAGGAGACAGAGTCACCATCACTTGCCGGGCCAGTCAGGGCAT | | GDRVTITCRASQGIS | | | |
| | | TAGCAGTTATGTAGCCTGGTATCAGCAAAAACCAGGGAAAGCC | | SYVAWYQQKPGKAPK | | | |
| | | CCTAAGCTCCTGATGTATGCTGCATCCAGTTTGCAGGGTGGGG | | LLMYAASSLQGGVPS | | | |
| | | TCCCATCAAGGTTCAGCGGCAGTGGATCTGGGACAGAATTCAC | | RFSGSGSGTEFTLTI | | | |
| | | TCTCACAATCAGCAGCCTGCAGCCTGAAGATTTTGCAACTTAT | | SSLQPEDFATYYCQQ | | | |
| | | TACTGTCAACAGCTTAGTGGTTACTTCACTTTCGGCGGAGGGA | | LSGYFTFGGGTKVEI | | | |
| | | CCAAGGTGGAAATCAAA | | K | | | |
| ADI-81745 | 2939 | TCGTATGTGCTGACTCAGCCACCCTCGGTGTCGGTGGCCCCAG | 4811 | SYVLTQPPSVSVAPG | 5164 | 4 | 3 |
| | | GACAGACGGCCAGGATTACCTGTGGGGAAGACGGCATTGGAAG | | QTARITCGEDGIGSK | | | |
| | | TAAAAGTGTACACTGGTACCAGCAGAAGCCAGGCCAGGCCCCT | | SVHWYQQKPGQAPVL | | | |
| | | GTGCTGGTCGTCTATGATGATAGCGACCGGCCCTCAGGGATCC | | VVYDDSDRPSGIPER | | | |
| | | CTGAGCGATTCTCTGGCTCCAACTCTGGGAACACGGCCACCCT | | FSGSNSGNTATLTIS | | | |
| | | GACCATCAGCAGGGTCGAAGCCGGGGATGAGGCCGACTATTAC | | RVEAGDEADYYCQVW | | | |
| | | TGTCAGGTGTGGGGTTTTAGTGGTGATCACTGGGTGTTCGGCG | | GFSGDHWVFGGGTKL | | | |
| | | GAGGGACCAAGCTCACCGTCCTA | | TVL | | | |
| ADI-81630 | 2939 | TCGTCTGAGCTGAGTCAGCAGCCCTCAGTGTCTGGGGCCCCAG | 4812 | SSELSQQPSVSGAPG | 5165 | 4 | 4 |
| | | GGCAGAGGGTCATCATCTCCTGCACTGGGAGCAACTCCAACAT | | QRVIISCTGSNSNIG | | | |
| | | CGGGGCAGGTTATGATGTACACTGGTACCAGCAGCTTCCAGGA | | AGYDVHWYQQLPGTA | | | |
| | | ACAGCCCCCAAACTCCTCATCTATGGTAACACCAATCGGCCCT | | PKLLIYGNTNRPSGV | | | |
| | | CAGGGGTCCCTGACCGGTTCTCTGGCTCCAAGTCTGACACCTC | | PDRFSGSKSDTSASL | | | |
| | | AGCCTCCCTGGCCATCACTGGGCTCCAGGCTGAGGATGAGGCT | | AITGLQAEDEADYYC | | | |
| | | GATTATTACTGCCAGTCCTATGACAGCGGCCTGACTGGTTCGC | | QSYDSGLTGSLFGGG | | | |
| | | TTTTCGGCGGAGGGACCAAGCTCACCGTCCTA | | TKLTVL | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81660 | IGLV1-40 | QSGLTQ | 3650 | TGSNSN | 2143 | WYQQLP | 3992 | VNSNRP | 4113 | GVPDRF | 4281 | QSYDSS | 2840 | FGGGTK |
| | | PPSVSG | | IGAGYD | | GTAPKL | | S | | SGSKSG | | LSDSV | | VTVL |
| | | APGQRV | | VH | | LIS | | | | TSASLA | | | | |
| | | TISC | | | | | | | | ITGLQA | | | | |
| | | | | | | | | | | EDEAAY | | | | |
| | | | | | | | | | | YC | | | | |
| ADI-81733 | IGLV3-25 | SHVLTQ | 3651 | SGDAFP | 2092 | WYQQKP | 2304 | KDNERP | 4049 | GIPERF | 4282 | QTADSS | 4481 | FGGGTK |
| | | PPSVSV | | NQYAY | | GQAPVL | | S | | SGSSSG | | RVV | | LTVL |
| | | SPRQTA | | | | VIY | | | | TTVTLT | | | | |
| | | RITC | | | | | | | | ISGVQA | | | | |
| | | | | | | | | | | EDEADY | | | | |
| | | | | | | | | | | YC | | | | |
| ADI-81736 | IGLV6-57 | QPMLTQ | 3652 | TRNSGS | 3853 | WYQQRP | 2366 | EDKQRP | 4131 | GVPDRF | 2574 | QSSHNS | 4482 | FGGGTK |
| | | PHSVSE | | IASNYV | | GSAPTT | | S | | SGSIDS | | KGV | | LTVL |
| | | SPGKTV | | Q | | VIF | | | | SSNSAS | | | | |
| | | TISC | | | | | | | | LTISGL | | | | |
| | | | | | | | | | | KTEDEA | | | | |
| | | | | | | | | | | DYYC | | | | |
| ADI-81759 | IGKV1-NL1 | EIEMTQ | 3653 | RASQGI | 2062 | WYQQKP | 2259 | AASTLE | 2398 | GVPSRF | 4283 | QQYYSN | 4483 | FGQGTK |
| | | SPSSLF | | SNSLA | | GKAPKL | | S | | SGSGSG | | LGRT | | VDIK |
| | | ASVGDR | | | | LLY | | | | TDFTLT | | | | |
| | | VTITC | | | | | | | | ISSLQP | | | | |
| | | | | | | | | | | EDFSTY | | | | |
| | | | | | | | | | | YC | | | | |
| ADI-81542 | IGKV1D-39 | EIQMTQ | 3654 | RASQSI | 2134 | WYQQKP | 2371 | AASSLQ | 2391 | GVPSRF | 4284 | QQSYSH | 4484 | FGQGTK |
| | | SPSSLS | | SRYLN | | GKAPEL | | S | | SGSGSG | | PPEGS | | VEIK |
| | | ASIGDR | | | | LIY | | | | TDFTLT | | | | |
| | | VTITC | | | | | | | | ISSLHP | | | | |
| | | | | | | | | | | EDFATY | | | | |
| | | | | | | | | | | YC | | | | |
| ADI-81639 | IGKV1-27 | DMRMTQ | 3655 | RASQGI | 2187 | WYQQKP | 4001 | GASTLQ | 2447 | GVSYRF | 4285 | QKYNTA | 4485 | FGQGTK |
| | | SPSSLS | | SNYLA | | GKVPKL | | S | | SGSGSG | | PCT | | VEIK |
| | | ASVGDR | | | | LIH | | | | TDFTLT | | | | |
| | | VTITC | | | | | | | | ISSLQP | | | | |
| | | | | | | | | | | EDVATY | | | | |
| | | | | | | | | | | YC | | | | |
| ADI-81815 | IGKV1-NL1 | EIRLTQ | 3656 | RASQDI | 2113 | WYQQKP | 2259 | AASTLE | 2398 | GVPSRF | 4286 | QQYYST | 4486 | FGQGTK |
| | | SPSSLP | | SNSLA | | GKAPKL | | S | | SGSASG | | PPFT | | VEIK |
| | | ASVGDR | | | | LLY | | | | TDYTLT | | | | |
| | | VTITC | | | | | | | | ISSLQP | | | | |
| | | | | | | | | | | EDFATY | | | | |
| | | | | | | | | | | YC | | | | |
| ADI-81852 | IGLV1-44 | QHVLTQ | 3657 | SGSSSN | 2162 | WYQQLP | 4002 | INNRRP | 4132 | GVPDRF | 2615 | ASWDDS | 4487 | FGGGTK |
| | | PPSASG | | IGSNSV | | GTAPKP | | S | | SGSKSG | | LNGLV | | LTVL |
| | | TPGQRV | | N | | LIY | | | | TSASLA | | | | |
| | | TISC | | | | | | | | ISGLQS | | | | |
| | | | | | | | | | | EDEADY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81660 | 2947 | CAGTCAGGGCTGACTCAGCCGCCCTCAGTGTCTGGAGCCCCAG | 4813 | QSGLTQPPSVSGAPG | 5166 | 4 | 4 |
| | | GGCAGAGGGTCACCATCTCCTGCACTGGGAGCAACTCCAACAT | | QRVTISCTGSNSNIG | | | |
| | | CGGGGCAGGTTATGATGTACACTGGTACCAACAACTTCCAGGA | | AGYDVHWYQQLPGTA | | | |
| | | ACAGCCCCCAAACTCCTCATCTCTGTTAACAGCAATCGGCCCT | | PKLLISVNSNRPSGV | | | |
| | | CAGGGGTCCCTGACCGATTCTCTGGCTCCAAGTCTGGCACCTC | | PDRFSGSKSGTSASL | | | |
| | | AGCCTCCCTGGCCATCACTGGGCTCCAGGCTGAGGATGAGGCT | | AITGLQAEDEAAYYC | | | |
| | | GCTTATTACTGCCAGTCCTATGACAGCAGCCTGAGTGATTCGG | | QSYDSSLSDSVFGGG | | | |
| | | TGTTCGGCGGAGGGACCAAGGTCACCGTCCTA | | TKVTVL | | | |
| ADI-81733 | 2939 | TCGCATGTGCTGACTCAGCCACCCTCGGTGTCGGTGTCTCCAA | 4814 | SHVLTQPPSVSVSPR | 5167 | 4 | 4 |
| | | GACAGACGGCCAGGATCACCTGCTCTGGAGATGCATTTCCAAA | | QTARITCSGDAFPNQ | | | |
| | | CCAATATGCTTATTGGTACCAACAGAAGCCAGGCCAGGCCCCT | | YAYWYQQKPGQAPVL | | | |
| | | GTGTTAGTGATATATAAAGACAATGAGAGGCCCTCAGGGATCC | | VIYKDNERPSGIPER | | | |
| | | CTGAGCGATTCTCTGGCTCCAGTTCAGGGACAACAGTCACGTT | | FSGSSSGTTVTLTIS | | | |
| | | GACCATCAGTGGAGTCCAGGCAGAAGACGAGGCTGACTATTAC | | GVQAEDEADYYCQTA | | | |
| | | TGTCAAACAGCAGACAGCAGTCGTGTGGTATTCGGCGGAGGGA | | DSSRVVFGGGTKLTV | | | |
| | | CCAAGCTCACCGTCCTA | | L | | | |
| ADI-81736 | 2939 | CAGCCTATGCTGACTCAGCCCCACTCTGTGTCGGAGTCTCCGG | 4815 | QPMLTQPHSVSESPG | 5168 | 4 | 4 |
| | | GGAAGACGGTTACCATCTCCTGCACCCGCAACAGTGGCAGCAT | | KTVTISCTRNSGSIA | | | |
| | | TGCCAGCAACTATGTGCAGTGGTACCAGCAGCGCCCGGGCAGT | | SNYVQWYQQRPGSAP | | | |
| | | GCCCCCACCACTGTGATCTTTGAAGATAAACAAAGACCCTCTG | | TTVIFEDKQRPSGVP | | | |
| | | GTGTCCCTGATCGGTTCTCTGGCTCCATCGACAGCTCCTCCAA | | DRFSGSIDSSSNSAS | | | |
| | | CTCTGCCTCCCTCACCATCTCTGGACTGAAGACTGAGGACGAA | | LTISGLKTEDEADYY | | | |
| | | GCTGACTACTACTGTCAGTCTTCTCATAACAGTAAGGGGGTGT | | CQSSHNSKGVFGGGT | | | |
| | | TCGGCGGAGGGACCAAGCTCACCGTCCTA | | KLTVL | | | |
| ADI-81759 | 2944 | GAAATTGAGATGACGCAGTCTCCATCCTCCCTGTTTGCATCTG | 4816 | EIEMTQSPSSLFASV | 5169 | 4 | 4 |
| | | TAGGAGACAGAGTCACCATCACTTGCCGGGCGAGTCAGGGTAT | | GDRVTITCRASQGIS | | | |
| | | TAGCAATTCTTTAGCCTGGTATCAGCAGAAACCAGGGAAAGCC | | NSLAWYQQKPGKAPK | | | |
| | | CCTAAGCTCCTGCTCTATGCTGCATCCACATTGGAAAGTGGGG | | LLLYAASTLESGVPS | | | |
| | | TCCCTTCCAGGTTCAGTGGCAGTGGATCTGGGACGGATTTCAC | | RFSGSGSGTDFTLTI | | | |
| | | TCTCACCATCAGCAGCCTGCAGCCTGAAGATTTTTCAACTTAT | | SSLQPEDFSTYYCQQ | | | |
| | | TACTGTCAACAGTATTATAGTAACCTCGGGAGGACGTTCGGCC | | YYSNLGRTFGQGTKV | | | |
| | | AAGGGACCAAGGTGGATATCAAA | | DIK | | | |
| ADI-81542 | 2937 | GAAATCCAGATGACCCAGTCTCCATCCTCCCTGTCTGCATCTA | 4817 | EIQMTQSPSSLSASI | 5170 | 4 | 4 |
| | | TAGGAGACAGAGTCACCATCACTTGCCGGGCAAGTCAGAGCAT | | GDRVTITCRASQSIS | | | |
| | | TAGCAGATATTTAAATTGGTATCAACAAAAACCTGGGAAAGCC | | RYLNWYQQKPGKAPE | | | |
| | | CCTGAGCTCCTGATCTATGCTGCATCCAGTTTGCAAAGTGGGG | | LLIYAASSLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCACCCTGAAGATTTTGCAACTTAC | | SSLHPEDFATYYCQQ | | | |
| | | TACTGTCAACAGAGTTACAGTCACCCTCCGGAGGGCAGTTTTG | | SYSHPPEGSFGQGTK | | | |
| | | GCCAGGGGACCAAGGTGGAAATCAAA | | VEIK | | | |
| ADI-81639 | 2937 | GACATGCGGATGACCCAGTCTCCATCTTCCCTGTCTGCATCTG | 4818 | DMRMTQSPSSLSASV | 5171 | 4 | 4 |
| | | TAGGAGACAGAGTCACCATCACTTGCCGGGCGAGTCAGGGCAT | | GDRVTITCRASQGIS | | | |
| | | AAGCAATTATTTAGCCTGGTATCAGCAGAAACCAGGGAAAGTT | | NYLAWYQQKPGKVPK | | | |
| | | CCTAAGCTCCTGATCCATGGTGCATCCACTTTGCAATCGGGGG | | LLIHGASTLQSGVSY | | | |
| | | TCTCATATAGGTTCAGTGGCAGTGGATCTGGGACAGATTTCAC | | RFSGSGSGTDFTLTI | | | |
| | | TCTCACCATCAGCAGCCTGCAGCCTGAAGATGTTGCAACTTAT | | SSLQPEDVATYYCQK | | | |
| | | TACTGTCAAAAGTACAACACTGCCCCGTGCACGTTCGGCCAAG | | YNTAPCTFGQGTKVE | | | |
| | | GGACCAAGGTGGAGATCAAA | | IK | | | |
| ADI-81815 | 2937 | GAAATCCGGTTGACCCAGTCTCCATCCTCCCTGCCTGCATCTG | 4819 | EIRLTQSPSSLPASV | 5172 | 4 | 4 |
| | | TAGGAGACAGAGTCACCATCACTTGCCGGGCGAGTCAGGACAT | | GDRVTITCRASQDIS | | | |
| | | TAGCAATTCTTTAGCCTGGTATCAGCAGAAACCAGGGAAAGCC | | NSLAWYQQKPGKAPK | | | |
| | | CCTAAGCTCCTGCTCTATGCTGCATCCACATTGGAAAGTGGGG | | LLLYAASTLESGVPS | | | |
| | | TCCCATCCAGGTTCAGTGGCAGTGCATCTGGGACGGATTACAC | | RFSGSASGTDYTLTI | | | |
| | | TCTCACCATCAGCAGCCTGCAGCCTGAAGATTTTGCAACTTAT | | SSLQPEDFATYYCQQ | | | |
| | | TACTGTCAACAGTATTATAGTACCCCTCCGTTCACTTTTGGCC | | YYSTPPFTFGQGTKV | | | |
| | | AGGGGACCAAGGTGGAGATCAAA | | EIK | | | |
| ADI-81852 | 2939 | CAGCATGTGCTGACTCAGCCACCCTCAGCGTCTGGGACCCCCG | 4820 | QHVLTQPPSASGTPG | 5173 | 4 | 4 |
| | | GGCAGAGGGTCACCATCTCTTGTTCTGGAAGCAGCTCCAACAT | | QRVTISCSGSSSNIG | | | |
| | | CGGAAGTAATTCTGTAAACTGGTACCAGCAGCTCCCAGGAACG | | SNSVNWYQQLPGTAP | | | |
| | | GCCCCCAAACCCCTCATCTATATTAATAATCGGCGGCCCTCAG | | KPLIYINNRRPSGVP | | | |
| | | GGGTCCCTGACCGATTCTCTGGCTCCAAGTCTGGCACCTCAGC | | DRFSGSKSGTSASLA | | | |
| | | CTCCCTGGCCATCAGTGGGCTCCAGTCTGAGGATGAGGCTGAT | | ISGLQSEDEADYYCA | | | |
| | | TATTACTGTGCATCATGGGATGACAGCCTGAATGGTTTGGTTT | | SWDDSLNGLVFGGGT | | | |
| | | TCGGCGGAGGGACCAAGCTCACCGTCCTA | | KLTVL | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81853 | IGKV1-5 | DIQMTQ | 3658 | RASQSI | 2052 | WYQQKP | 2263 | KASTLE | 2518 | GVPSRF | 2532 | QQYNSF | 4488 | FGQGTK |
| | | SPSTLS | | SRWLA | | GKAPNL | | S | | SGSGSG | | SWT | | VEIK |
| | | ASVGDR | | | | LIY | | | | TEFTLT | | | | |
| | | VAITC | | | | | | | | ISSLQP | | | | |
| | | | | | | | | | | DDFATY | | | | |
| | | | | | | | | | | YC | | | | |
| ADI-81647 | IGLV1-40 | QSVLTQ | 1948 | TGSGSN | 3854 | WYQQLP | 4000 | RNSNRP | 4133 | GVPDRF | 4287 | QSYDSS | 4489 | FGTGTK |
| | | PPSVSG | | IGAGYD | | GTAPKL | | S | | SGSMSG | | LSGSI | | LTVL |
| | | APGQRV | | VH | | LIF | | | | TSASLA | | | | |
| | | TISC | | | | | | | | ITGLQA | | | | |
| | | | | | | | | | | EDEADY | | | | |
| | | | | | | | | | | YC | | | | |
| ADI-81689 | IGKV3D-15 | ETTLTQ | 2022 | RASQTI | 3855 | WYQQKP | 2258 | GASTRA | 2383 | GIPARF | 4288 | QHYDNW | 4490 | FGQGTK |
| | | SPATLS | | SNNLA | | GQAPRL | | T | | SGSGSG | | PPEYT | | VEIK |
| | | VSPGER | | | | LIY | | | | TEFTLT | | | | |
| | | ATLSC | | | | | | | | ISSLQS | | | | |
| | | | | | | | | | | EDSAVY | | | | |
| | | | | | | | | | | YC | | | | |
| ADI-81550 | IGLV1-40 | QSVVTQ | 1972 | TGSNSN | 2143 | WYQQLP | 2314 | RNTNRP | 4054 | GVPDRF | 2599 | QSYDSS | 4491 | FGGGTK |
| | | PPSVSG | | IGAGYD | | GTAPKL | | S | | SGSKSG | | LTGSL | | LTVL |
| | | APGQRV | | VH | | LIY | | | | TSASLA | | | | |
| | | TISC | | | | | | | | ITGLQA | | | | |
| | | | | | | | | | | EDEADY | | | | |
| | | | | | | | | | | YC | | | | |
| ADI-81552 | IGLV1-40 | QSVVTQ | 1972 | TGSNSN | 2143 | WYQQLP | 2314 | RNTNRP | 4054 | GVPDRF | 2599 | QSYDSS | 4491 | FGGGTQ |
| | | PPSVSG | | IGAGYD | | GTAPKL | | S | | SGSKSG | | LTGSL | | LTVL |
| | | APGQRV | | VH | | LIY | | | | TSASLA | | | | |
| | | TISC | | | | | | | | ITGLQA | | | | |
| | | | | | | | | | | EDEADY | | | | |
| | | | | | | | | | | Y | | | | |
| ADI-81601 | IGLV1-40 | QSVLTQ | 1948 | TGSNSN | 2196 | WYQQLP | 2314 | GNTNRP | 2484 | GVPDRF | 2622 | QSYDSS | 2862 | FGGGTK |
| | | PPSVSG | | IGAGYD | | GTAPKL | | S | | SGSKSD | | LSGVL | | LTVL |
| | | APGQRV | | IH | | LIY | | | | TSASLA | | | | |
| | | TISC | | | | | | | | ITGLQA | | | | |
| | | | | | | | | | | EDEADY | | | | |
| | | | | | | | | | | YC | | | | |
| ADI-81653 | IGKV3D-15 | EMTLTQ | 3659 | RASQSV | 2100 | WYQQKP | 4003 | GASTRA | 2383 | GIPARF | 4289 | QQYNKW | 4492 | FGQGTR |
| | | SPATLS | | SSSLA | | GQAPRL | | T | | SGSGSG | | PPIT | | LEIK |
| | | VSPGER | | | | LIH | | | | TEFTLT | | | | |
| | | ATLFC | | | | | | | | ISSLQS | | | | |
| | | | | | | | | | | EDVAVY | | | | |
| | | | | | | | | | | YC | | | | |
| ADI-81661 | IGLV1-40 | QSVVTQ | 1972 | AGSGSN | 3856 | WYQQLP | 2314 | GNNNRP | 2492 | GVPDRF | 4237 | QSYDSS | 4493 | FGGGTK |
| | | PPSVSG | | IGAGYD | | GTAPKL | | S | | SGSKSA | | LSDVI | | LTVL |
| | | APGQRV | | VH | | LIY | | | | TSASLA | | | | |
| | | TISC | | | | | | | | ITGLQA | | | | |
| | | | | | | | | | | EDEADY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81853 | 2937 | GACATCCAGATGACGCAGTCTCCTTCCACCCTGTCTGCATCTG | 4821 | DIQMTQSPSTLSASV | 5174 | 4 | 4 |
| | | TAGGAGACAGAGTCGCCATCACTTGCCGGGCCAGTCAGAGTAT | | GDRVAITCRASQSIS | | | |
| | | TAGTAGGTGGTTGGCCTGGTATCAGCAGAAACCAGGGAAAGCC | | RWLAWYQQKPGKAPN | | | |
| | | CCTAACCTCCTGATTTATAAGGCGTCTACTTTAGAAAGTGGGG | | LLIYKASTLESGVPS | | | |
| | | TCCCATCAAGGTTCAGCGGCAGTGGATCTGGGACAGAATTCAC | | RFSGSGSGTEFTLTI | | | |
| | | TCTCACCATCAGCAGCCTGCAGCCTGATGATTTTGCAACTTAT | | SSLQPDDFATYYCQQ | | | |
| | | TACTGCCAACAGTATAATAGTTTTTCGTGGACGTTCGGCCAAG | | YNSFSWTFGQGTKVE | | | |
| | | GGACCAAGGTGGAAATCAAA | | IK | | | |
| ADI-81647 | 2949 | CAGTCTGTGCTGACGCAGCCGCCCTCAGTGTCTGGGGCCCCAG | 4822 | QSVLTQPPSVSGAPG | 5175 | 4 | 4 |
| | | GGCAGAGGGTCACCATCTCCTGCACTGGGAGCGGCTCCAACAT | | QRVTISCTGSGSNIG | | | |
| | | CGGGGCAGGTTATGATGTACACTGGTATCAGCAGCTTCCAGGA | | AGYDVHWYQQLPGTA | | | |
| | | ACAGCCCCCAAACTCCTCATCTTTCGTAACTCCAATCGGCCCT | | PKLLIFRNSNRPSGV | | | |
| | | CAGGGGTCCCTGACCGATTCTCTGGCTCCATGTCTGGCACCTC | | PDRFSGSMSGTSASL | | | |
| | | AGCCTCCCTGGCCATCACTGGGCTCCAGGCTGAGGATGAGGCT | | AITGLQAEDEADYYC | | | |
| | | GATTATTACTGCCAGTCGTATGACAGCAGCCTGAGTGGATCGA | | QSYDSSLSGSIFGTG | | | |
| | | TCTTCGGAACTGGGACCAAGCTCACCGTCCTA | | TKLTVL | | | |
| ADI-81689 | 2937 | GAAACGACACTCACGCAGTCTCCAGCCACCCTGTCTGTGTCTC | 4823 | ETTLTQSPATLSVSP | 5176 | 4 | 4 |
| | | CAGGGGAAAGAGCCACTCTCTCCTGCAGGGCCAGTCAGACTAT | | GERATLSCRASQTIS | | | |
| | | TTCCAACAACTTAGCCTGGTACCAGCAGAAACCTGGCCAGGCT | | NNLAWYQQKPGQAPR | | | |
| | | CCCAGGCTCCTCATCTATGGTGCATCTACCAGGGCCACTGGTA | | LLIYGASTRATGIPA | | | |
| | | TCCCAGCCAGGTTCAGTGGCAGTGGGTCTGGGACAGAGTTCAC | | RFSGSGSGTEFTLTI | | | |
| | | TCTCACCATCAGCAGCCTGCAGTCTGAAGATTCTGCAGTTTAT | | SSLQSEDSAVYYCQH | | | |
| | | TACTGTCAACACTATGATAACTGGCCTCCGGAATACACTTTTG | | YDNWPPEYTFGQGTK | | | |
| | | GCCAGGGGACCAAGGTGGAGATCAAA | | VEIK | | | |
| ADI-81550 | 2939 | CAGTCTGTGGTGACGCAGCCGCCCTCAGTGTCTGGGGCCCCAG | 4824 | QSVVTQPPSVSGAPG | 5177 | 4 | 3 |
| | | GGCAGAGGGTCACCATCTCCTGCACTGGGAGCAACTCCAACAT | | QRVTISCTGSNSNIG | | | |
| | | CGGGGCAGGTTATGATGTACACTGGTACCAGCAACTTCCAGGA | | AGYDVHWYQQLPGTA | | | |
| | | ACAGCCCCCAAACTCCTCATCTATAGAAACACCAATCGGCCCT | | PKLLIYRNTNRPSGV | | | |
| | | CAGGGGTCCCTGACCGATTCTCTGGGTCCAAGTCTGGCACCTC | | PDRFSGSKSGTSASL | | | |
| | | AGCCTCCCTGGCCATTACTGGGCTCCAGGCTGAGGATGAGGCT | | AITGLQAEDEADYYC | | | |
| | | GATTATTACTGCCAGTCCTATGACAGCAGCCTGACTGGTTCGC | | QSYDSSLTGSLFGGG | | | |
| | | TATTCGGCGGAGGGACCAAGCTCACCGTCCTA | | TKLTVL | | | |
| ADI-81552 | 2948 | CAGTCAGTGGTGACGCAGCCGCCCTCAGTGTCTGGGGCCCCAG | 4825 | QSVVTQPPSVSGAPG | 5178 | 4 | 3 |
| | | GGCAGAGGGTCACCATCTCCTGCACTGGGAGCAACTCCAACAT | | QRVTISCTGSNSNIG | | | |
| | | CGGGGCAGGTTATGATGTACACTGGTACCAGCAGCTTCCAGGA | | AGYDVHWYQQLPGTA | | | |
| | | ACAGCCCCCAAACTCCTCATCTATAGAAACACCAATCGGCCCT | | PKLLIYRNTNRPSGV | | | |
| | | CAGGGGTCCCTGACCGATTCTCTGGGTCCAAGTCTGGCACCTC | | PDRFSGSKSGTSASL | | | |
| | | AGCCTCCCTGGCCATCACTGGGCTCCAGGCTGAGGATGAGGCT | | AITGLQAEDEADYYC | | | |
| | | GATTATTACTGCCAGTCCTATGACAGCAGCCTGACTGGTTCGC | | QSYDSSLTGSLFGGG | | | |
| | | TGTTCGGCGGAGGCACCCAGCTGACCGTCCTC | | TQLTVL | | | |
| ADI-81601 | 2939 | CAGTCTGTCCTGACTCAGCCGCCCTCAGTATCTGGGGCCCCAG | 4826 | QSVLTQPPSVSGAPG | 5179 | 4 | 4 |
| | | GGCAGAGGGTCACCATCTCCTGCACTGGGAGCAACTCCAACAT | | QRVTISCTGSNSNIG | | | |
| | | CGGAGCAGGTTATGATATACACTGGTACCAGCAGCTTCCAGGA | | AGYDIHWYQQLPGTA | | | |
| | | ACAGCCCCCAAACTCCTCATCTATGGTAACACCAATCGGCCCT | | PKLLIYGNTNRPSGV | | | |
| | | CAGGGGTCCCTGACCGATTCTCTGGCTCCAAGTCTGACACCTC | | PDRFSGSKSDTSASL | | | |
| | | AGCCTCCCTGGCCATCACTGGGCTCCAGGCTGAGGATGAGGCT | | AITGLQAEDEADYYC | | | |
| | | GATTATTACTGCCAGTCTTATGACAGCAGCCTGAGTGGTGTGC | | QSYDSSLSGVLFGGG | | | |
| | | TATTCGGCGGAGGGACCAAGCTCACCGTCCTA | | TKLTVL | | | |
| ADI-81653 | 2940 | GAAATGACACTCACGCAGTCTCCAGCCACCCTGTCTGTGTCTC | 4827 | EMTLTQSPATLSVSP | 5180 | 4 | 4 |
| | | CAGGGGAAAGAGCCACCCTCTTCTGCAGGGCCAGTCAGAGTGT | | GERATLFCRASQSVS | | | |
| | | TAGCAGCAGCTTAGCCTGGTACCAGCAGAAACCTGGCCAGGCT | | SSLAWYQQKPGQAPR | | | |
| | | CCCAGGCTCCTCATCCATGGTGCATCCACCAGGGCCACTGGTA | | LLIHGASTRATGIPA | | | |
| | | TCCCAGCCAGGTTCAGTGGCAGTGGGTCTGGGACAGAGTTCAC | | RFSGSGSGTEFTLTI | | | |
| | | TCTCACCATCAGCAGCCTGCAGTCTGAAGATGTTGCAGTTTAT | | SSLQSEDVAVYYCQQ | | | |
| | | TACTGTCAGCAGTATAATAAGTGGCCTCCGATCACCTTCGGCC | | YNKWPPITFGQGTRL | | | |
| | | AAGGGACACGACTGGAGATTAAA | | EIK | | | |
| ADI-81661 | 2939 | CAGTCTGTCGTGACGCAGCCGCCCTCAGTGTCTGGGGCCCCAG | 4828 | QSVVTQPPSVSGAPG | 5181 | 4 | 4 |
| | | GGCAGAGGGTCACCATCTCCTGCGCTGGGAGCGGCTCCAACAT | | QRVTISCAGSGSNIG | | | |
| | | CGGGGCAGGTTACGATGTGCACTGGTACCAGCAACTTCCAGGA | | AGYDVHWYQQLPGTA | | | |
| | | ACAGCCCCCAAACTCCTCATCTATGGTAACAACAATCGGCCCT | | PKLLIYGNNNRPSGV | | | |
| | | CAGGGGTCCCTGACCGATTCTCTGGCTCCAAGTCTGCCACCTC | | PDRFSGSKSATSASL | | | |
| | | AGCCTCCCTGGCCATCACTGGACTCCAGGCTGAGGATGAGGCT | | AITGLQAEDEADYYC | | | |
| | | GATTATTACTGCCAGTCCTATGACAGCAGCCTGAGTGATGTGA | | QSYDSSLSDVIFGGG | | | |
| | | TATTCGGCGGAGGGACCAAGCTCACCGTCCTA | | TKLTVL | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81763 | IGKV1D-39 | EMTLTQ | 3660 | RASQSI | 2120 | WYQQKP | 2257 | ATSSLQ | 4134 | GVPSRF | 2534 | QQSSSN | 4494 | FGPGTK |
| | | SPPSLS | | GSYLN | | GKAPKL | | S | | SGSGSG | | FPT | | VDIK |
| | | ASVGDR | | | | LIY | | | | TDFTLT | | | | |
| | | VSITC | | | | | | | | ISSLQP | | | | |
| | | | | | | | | | | EDFATY | | | | |
| | | | | | | | | | | YC | | | | |
| ADI-81807 | IGLV1-40 | QSVLTQ | 1948 | TGSSSN | 3857 | WYQQLP | 2314 | TNTNRP | 4135 | GVPDRF | 2599 | QSYDTS | 4495 | FGGGTK |
| | | PPSVSG | | IGAGSD | | GTAPKL | | S | | SGSKSG | | LSGVV | | LTVL |
| | | APGQRV | | VH | | LIY | | | | TSASLA | | | | |
| | | TISC | | | | | | | | ITGLQA | | | | |
| | | | | | | | | | | EDEADY | | | | |
| | | | | | | | | | | YC | | | | |
| ADI-81849 | IGLV3-25 | QPVLTQ | 3661 | SGEALP | 3858 | WYQQKP | 2304 | KDTERP | 2459 | GIPERF | 4282 | QSADTS | 4496 | FGGGTK |
| | | PPSVSV | | MQYTY | | GQAPVL | | S | | SGSSSG | | GNYRGV | | LTVL |
| | | SPGQTA | | | | VIY | | | | TTVTLT | | | | |
| | | RITC | | | | | | | | ISGVQA | | | | |
| | | | | | | | | | | EDEADY | | | | |
| | | | | | | | | | | YC | | | | |
| ADI-81851 | IGKV3-20 | ETTLTQ | 3628 | RASQSV | 3859 | WYQQKP | 4004 | GASSRA | 4024 | GIPDRF | 2644 | QQYAFS | 4497 | FGGGTK |
| | | SPGTLS | | SSGYLA | | GQAPSL | | S | | SGSGSG | | PLT | | VEIK |
| | | LSPGER | | | | LIY | | | | TDFTLT | | | | |
| | | ATLSC | | | | | | | | ITRLEP | | | | |
| | | | | | | | | | | EDFAVY | | | | |
| | | | | | | | | | | YC | | | | |
| ADI-81747 | IGLV2-8 | QFVLTQ | 3662 | TGTSSD | 3860 | WYQQHP | 4005 | EVNKRP | 2394 | GVPDRF | 2538 | SSYAGS | 4498 | FGGGTK |
| | | PPSASG | | IGGYNY | | GKAPKL | | S | | SGSKSG | | NTVV | | LTVL |
| | | SPGQSV | | VS | | MAY | | | | NTASLT | | | | |
| | | TISC | | | | | | | | VSGLQA | | | | |
| | | | | | | | | | | EDEADY | | | | |
| | | | | | | | | | | YC | | | | |
| ADI-81753 | IGKV1-5 | EIGVTQ | 3663 | RASQSI | 2146 | WYQQKP | 2263 | KASLLE | 4136 | GVPSRF | 2532 | QLYNSD | 4499 | FGQGTK |
| | | SPSTLS | | STWLA | | GKAPNL | | S | | SGSGSG | | SWT | | VEIK |
| | | ASVGDR | | | | LIY | | | | TEFTLT | | | | |
| | | VTITC | | | | | | | | ISSLQP | | | | |
| | | | | | | | | | | DDFATY | | | | |
| | | | | | | | | | | YC | | | | |
| ADI-81874 | IGKV1-33 | DIVLTQ | 1957 | QATQDI | 3861 | WYQQKP | 2294 | DASNLE | 2416 | GVPSRF | 2537 | QQYDIF | 4500 | FGPGTK |
| | | SPSSLS | | NNSLN | | GKAPRL | | T | | SGSGSG | | IGFT | | VDIK |
| | | ASVGDR | | | | LIY | | | | TDFTFT | | | | |
| | | VTITC | | | | | | | | ISSLQP | | | | |
| | | | | | | | | | | EDIATY | | | | |
| | | | | | | | | | | YC | | | | |
| ADI-81879 | IGKV3D-15 | EMTLTQ | 1916 | RASQSI | 3862 | GQAPRL | 4006 | GASTRA | 2383 | GIPARF | 2569 | QHYNNW | 4501 | FGQGTK |
| | | SPATLS | | STTLA | | WYQQKP | | T | | SGSGSG | | TPT | | VEIK |
| | | VSPGER | | | | LMY | | | | TEFTLT | | | | |
| | | ATLSC | | | | | | | | ISSLQS | | | | |
| | | | | | | | | | | EDFAVY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81763 | 2950 | GAAATGACACTCACGCAGTCTCCACCCTCCCTGTCTGCATCTG | 4829 | EMTLTQSPPSLSASV | 5182 | 4 | 4 |
| | | TAGGAGACAGAGTCAGCATCACTTGCCGGGCAAGTCAGAGCAT | | GDRVSITCRASQSIG | | | |
| | | TGGCAGCTATTTAAATTGGTATCAGCAGAAACCAGGGAAAGCC | | SYLNWYQQKPGKAPK | | | |
| | | CCTAAGCTCCTGATCTATGCTACATCCAGTTTGCAAAGTGGGG | | LLIYATSSLQSGVPS | | | |
| | | TCCCCTCAAGGTTCAGTGGCAGTGGATCTGGGACGGATTTCAC | | RFSGSGSGTDFTLTI | | | |
| | | TCTCACCATCAGCAGTCTGCAACCTGAAGATTTTGCAACTTAC | | SSLQPEDFATYYCQQ | | | |
| | | TACTGTCAACAGAGTTCCAGTAATTTTCCCACTTTCGGCCCTG | | SSSNFPTFGPGTKVD | | | |
| | | GGACCAAGGTGGATATCAAA | | IK | | | |
| ADI-81807 | 2939 | CAGTCTGTGTTGACGCAGCCGCCCTCAGTGTCTGGGGCCCCAG | 4830 | QSVLTQPPSVSGAPG | 5183 | 4 | 3 |
| | | GGCAGAGGGTCACCATCTCCTGCACTGGGAGCAGCTCCAACAT | | QRVTISCTGSSSNIG | | | |
| | | CGGGGCAGGTTCTGATGTACACTGGTACCAGCAGCTTCCAGGA | | AGSDVHWYQQLPGTA | | | |
| | | ACAGCCCCCAAACTCCTCATCTATACTAACACCAATCGGCCCT | | PKLLIYTNTNRPSGV | | | |
| | | CAGGGGTCCCTGACCGATTCTCTGGCTCCAAGTCCGGCACCTC | | PDRFSGSKSGTSASL | | | |
| | | AGCCTCCCTGGCCATCACGGGGCTCCAGGCTGAGGATGAGGCT | | AITGLQAEDEADYYC | | | |
| | | GATTATTACTGCCAGTCCTATGACACCAGCTTGAGTGGGGTGG | | QSYDTSLSGVVFGGG | | | |
| | | TTTTCGGCGGAGGGACCAAGCTCACCGTCCTA | | TKLTVL | | | |
| ADI-81849 | 2939 | CAGCCTGTGCTGACTCAGCCCCCCTCGGTGTCAGTGTCCCCAG | 4831 | QPVLTQPPSVSVSPG | 5184 | 4 | 4 |
| | | GACAGACGGCCAGGATCACCTGCTCTGGAGAAGCATTGCCAAT | | QTARITCSGEALPMQ | | | |
| | | GCAATATACTTATTGGTACCAGCAGAAGCCAGGCCAGGCCCCT | | YTYWYQQKPGQAPVL | | | |
| | | GTATTGGTGATATATAAAGACACTGAGAGGCCCTCAGGGATCC | | VIYKDTERPSGIPER | | | |
| | | CTGAGCGATTCTCTGGCTCCAGCTCAGGGACAACAGTCACGTT | | FSGSSSGTTVTLTIS | | | |
| | | GACCATCAGTGGAGTCCAGGCAGAAGACGAGGCTGACTATTAC | | GVQAEDEADYYCQSA | | | |
| | | TGTCAATCAGCGGACACCAGTGGTAATTATCGGGGGGTTTTCG | | DTSGNYRGVFGGGTK | | | |
| | | GCGGAGGGACCAAGCTCACCGTCCTA | | LTVL | | | |
| ADI-81851 | 2936 | GAAACGACACTCACGCAGTCTCCAGGCACCCTGTCTTTGTCTC | 4832 | ETTLTQSPGTLSLSP | 5185 | 4 | 4 |
| | | CAGGGGAAAGAGCCACCCTCTCCTGCAGGGCCAGTCAGAGTGT | | GERATLSCRASQSVS | | | |
| | | TAGCAGCGGCTACTTAGCCTGGTACCAGCAGAAACCTGGCCAG | | SGYLAWYQQKPGQAP | | | |
| | | GCTCCCAGTCTCCTCATCTATGGTGCCTCCAGCAGGGCCTCTG | | SLLIYGASSRASGIP | | | |
| | | GCATCCCAGACAGGTTCAGTGGCAGTGGGTCTGGGACAGACTT | | DRFSGSGSGTDFTLT | | | |
| | | CACTCTCACCATCACCAGACTGGAGCCTGAAGATTTTGCAGTA | | ITRLEPEDFAVYYCQ | | | |
| | | TATTACTGTCAGCAGTATGCTTTCTCACCTCTCACTTTCGGCG | | QYAFSPLTFGGGTKV | | | |
| | | GAGGGACCAAGGTGGAAATCAAA | | EIK | | | |
| ADI-81747 | 2939 | CAGTTTGTGCTGACTCAGCCCCCCTCCGCGTCCGGGTCTCCTG | 4833 | QFVLTQPPSASGSPG | 5186 | 4 | 3 |
| | | GACAGTCAGTCACCATCTCCTGCACTGGAACCAGCAGTGACAT | | QSVTISCTGTSSDIG | | | |
| | | TGGTGGTTATAACTATGTCTCCTGGTACCAACAACACCCAGGC | | GYNYVSWYQQHPGKA | | | |
| | | AAAGCCCCCAAACTCATGGCTTATGAGGTCAATAAGCGGCCCT | | PKLMAYEVNKRPSGV | | | |
| | | CAGGGGTCCCTGATCGCTTCTCTGGCTCCAAGTCTGGCAACAC | | PDRFSGSKSGNTASL | | | |
| | | GGCCTCCCTGACCGTCTCTGGGCTCCAGGCTGAGGATGAGGCT | | TVSGLQAEDEADYYC | | | |
| | | GATTATTATTGCAGCTCATATGCCGGCAGCAACACTGTGGTTT | | SSYAGSNTVVFGGGT | | | |
| | | TCGGCGGAGGGACCAAGCTCACCGTCCTA | | KLTVL | | | |
| ADI-81753 | 2937 | GAAATCGGGGTGACCCAGTCTCCTTCCACCCTGTCTGCATCTG | 4834 | EIGVTQSPSTLSASV | 5187 | 4 | 3 |
| | | TAGGAGACAGAGTCACCATCACTTGCCGGGCCAGTCAGAGTAT | | GDRVTITCRASQSIS | | | |
| | | TAGTACCTGGTTGGCCTGGTATCAGCAGAAACCAGGGAAAGCC | | TWLAWYQQKPGKAPN | | | |
| | | CCTAACCTCCTGATCTATAAGGCGTCTCTTTTGGAAAGTGGGG | | LLIYKASLLESGVPS | | | |
| | | TCCCATCAAGGTTCAGCGGCAGTGGATCTGGGACAGAATTCAC | | RFSGSGSGTEFTLTI | | | |
| | | TCTCACCATCAGCAGCCTGCAGCCTGATGATTTTGCAACTTAT | | SSLQPDDFATYYCQL | | | |
| | | TACTGCCAACTGTATAATAGTGATTCGTGGACGTTCGGCCAAG | | YNSDSWTFGQGTKVE | | | |
| | | GGACCAAGGTGGAAATCAAA | | IK | | | |
| ADI-81874 | 2950 | GACATCGTGTTGACCCAGTCTCCATCCTCCCTGTCTGCATCTG | 4835 | DIVLTQSPSSLSASV | 5188 | 4 | 4 |
| | | TAGGAGACAGAGTCACCATCACTTGCCAGGCGACTCAGGACAT | | GDRVTITCQATQDIN | | | |
| | | TAACAACTCTTTAAATTGGTATCAGCAGAAACCAGGGAAAGCC | | NSLNWYQQKPGKAPR | | | |
| | | CCTAGGCTCCTGATCTACGATGCATCCAATTTGGAAACAGGGG | | LLIYDASNLETGVPS | | | |
| | | TCCCATCAAGGTTCAGTGGAAGTGGATCTGGGACAGATTTTAC | | RFSGSGSGTDFTFTI | | | |
| | | TTTCACCATCAGCAGCCTGCAGCCTGAGGATATTGCAACATAT | | SSLQPEDIATYYCQQ | | | |
| | | TACTGTCAACAGTATGACATTTTCGGGTTCACTTTCGGCCCTG | | YDIFGFTFGPGTKVD | | | |
| | | GGACCAAAGTGGATATCAAA | | IK | | | |
| ADI-81879 | 2937 | GAAATGACACTCACGCAGTCTCCAGCCACCCTGTCTGTGTCTC | 4836 | EMTLTQSPATLSVSP | 5189 | 4 | 4 |
| | | CAGGGGAAAGAGCCACCCTCTCCTGCAGGGCCAGTCAGAGTAT | | GERATLSCRASQSIS | | | |
| | | TAGCACCACCTTAGCCTGGTACCAGCAGAAACCTGGCCAGGCT | | TTLAWYQQKPGQAPR | | | |
| | | CCCAGACTCCTCATGTATGGTGCATCCACCAGGGCCACAGGTA | | LLMYGASTRATGIPA | | | |
| | | TTCCAGCCAGGTTCAGTGGCAGTGGGTCTGGGACAGAGTTCAC | | RFSGSGSGTEFTLTI | | | |
| | | TCTCACCATCAGCAGCCTGCAGTCTGAAGATTTTGCAGTTTAT | | SSLQSEDFAVYYCQH | | | |
| | | TACTGTCAGCACTATAATAACTGGACTCCGACGTTCGGCCAAG | | YNNWTPTFGQGTKVE | | | |
| | | GGACCAAGGTGGAAATCAAA | | IK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81547 | IGKV1D-39 | DIQMTQ | 1878 | RASQSI | 2117 | WYQQKP | 2257 | DASSLQ | 2389 | GVPSRF | 4290 | QQSYNT | 2702 | FGQGTK |
| | | SPSSLS | | RSYLN | | GKAPKL | | S | | FGSGSG | | PPWT | | VEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81556 | IGLV1-40 | QSVLTQ | 1948 | TGSNSN | 2143 | WYQQFP | 4007 | RNSNRP | 4133 | GVPDRF | 2599 | QSYDSG | 4502 | FGGGTK |
| | | PPSVSG | | IGAGYD | | GTAPKL | | S | | SGSKSG | | LSGSV | | LTVL |
| | | APGQRV | | VH | | LIF | | | | TSASLA | | | | |
| | | TISC | | | | | | | | ITGLQA | | | | |
| | | | | | | | | | | EDEADY | | | | |
| | | | | | | | | | | YC | | | | |
| ADI-81610 | IGLV1-40 | QSVLTQ | 1948 | TGSNSN | 2143 | WYQQLP | 2314 | RNTNRP | 4054 | GVPDRF | 4291 | QSYDSS | 2908 | FGGGTK |
| | | PPSVSG | | IGAGYD | | GTAPKL | | S | | SGSKSG | | LSGSV | | VTVL |
| | | APGQRV | | VH | | LIY | | | | ASASLA | | | | |
| | | TISC | | | | | | | | ITGLQA | | | | |
| | | | | | | | | | | EDEADY | | | | |
| | | | | | | | | | | YC | | | | |
| ADI-81764 | IGKV1D-39 | DIAMTQ | 3664 | RASQSI | 3863 | WYQQKP | 2257 | AASTLQ | 2392 | GVPSRF | 2534 | QQSYTT | 4503 | FGQGTK |
| | | SPSSLS | | GIYLN | | GKAPKL | | S | | SGSGSG | | PTWT | | VDIK |
| | | ASVGDR | | | | LIY | | | | TDFTLT | | | | |
| | | VTFTC | | | | | | | | ISSLQP | | | | |
| | | | | | | | | | | EDFATY | | | | |
| | | | | | | | | | | YC | | | | |
| ADI-81868 | IGLV1-40 | QSVLTQ | 2027 | TGSDSN | 3763 | WYQQLA | 4008 | GNSNRP | 2477 | GVPDRF | 2599 | QSFDSS | 4504 | FGTGTK |
| | | PPSVSG | | IGAGYD | | GTAPKL | | S | | SGSKSG | | LSGPNY | | LTVL |
| | | APGQRV | | VH | | LIY | | | | TSASLA | | V | | |
| | | IISC | | | | | | | | ITGLQA | | | | |
| | | | | | | | | | | EDEADY | | | | |
| | | | | | | | | | | YC | | | | |
| ADI-81876 | IGLV6-57 | NFMLTQ | 1992 | TRSSGG | 3864 | WYQQRP | 4009 | EDNQRP | 2431 | GVPDRF | 4292 | QSYDTS | 4505 | FGGGTK |
| | | PHSVSE | | IVNNYV | | GSSPTT | | S | | SGSIDS | | NHGV | | LTVL |
| | | SPGKTV | | Q | | VIY | | | | SSNSAS | | | | |
| | | TISC | | | | | | | | LTISGL | | | | |
| | | | | | | | | | | KTEDEA | | | | |
| | | | | | | | | | | VYYC | | | | |
| ADI-81800 | IGKV3D-15 | EMTMTQ | 2003 | RASQSI | 3862 | WYQQKP | 4006 | GASTRA | 2383 | GIPARF | 2569 | QQYNNW | 4506 | FGQGTK |
| | | SPATLS | | STTLA | | GQAPRL | | T | | SGSGSG | | TPT | | VEIK |
| | | VSPGER | | | | LMY | | | | TEFTLT | | | | |
| | | ATLSC | | | | | | | | ISSLQS | | | | |
| | | | | | | | | | | EDFAVY | | | | |
| | | | | | | | | | | YC | | | | |
| ADI-81892 | IGKV1D-39 | EMTLTQ | 1914 | RPSQSI | 3865 | WYQQKP | 2275 | AASSLQ | 2391 | GVPSRF | 4293 | QQNYNS | 4470 | FGGGTK |
| | | SPSSLS | | SSFLN | | GRAPKL | | S | | SGSESG | | PLT | | VEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81547 | 2937 | GACATCCAGATGACCCAGTCTCCATCCTCCCTGTCTGCATCTG | 4837 | DIQMTQSPSSLSASV | 5190 | 4 | 3 |
| | | TAGGAGACAGAGTCACCATCACTTGCCGGGCAAGTCAGAGCAT | | GDRVTITCRASQSIR | | | |
| | | TAGGAGCTATTTAAATTGGTATCAGCAGAAACCAGGGAAAGCC | | SYLNWYQQKPGKAPK | | | |
| | | CCTAAGCTCCTGATCTATGATGCATCCAGTTTGCAAAGTGGGG | | LLIYDASSLQSGVPS | | | |
| | | TCCCATCAAGGTTCTTTGGCAGTGGATCTGGGACAGATTTCAC | | RFFGSGSGTDFTLTI | | | |
| | | TCTCACCATCAGCAGTCTGCAACCTGAAGATTTTGCAACTTAC | | SSLQPEDFATYYCQQ | | | |
| | | TACTGTCAACAGAGTTACAATACCCCCCCGTGGACGTTCGGCC | | SYNTPPWTFGQGTKV | | | |
| | | AAGGGACCAAGGTGGAGATCAAA | | EIK | | | |
| ADI-81556 | 2939 | CAGTCTGTGTTGACGCAGCCGCCCTCAGTGTCTGGGGCCCCAG | 4838 | QSVLTQPPSVSGAPG | 5191 | 4 | 4 |
| | | GGCAGAGGGTCACCATCTCCTGCACTGGGAGCAACTCCAACAT | | QRVTISCTGSNSNIG | | | |
| | | CGGGGCAGGTTATGATGTACACTGGTACCAGCAGTTTCCAGGA | | AGYDVHWYQQFPGTA | | | |
| | | ACAGCCCCCAAACTCCTCATCTTTCGTAACAGCAATCGACCCT | | PKLLIFRNSNRPSGV | | | |
| | | CAGGGGTCCCTGACCGATTCTCTGGCTCCAAGTCTGGCACCTC | | PDRFSGSKSGTSASL | | | |
| | | AGCCTCCCTGGCCATCACTGGGCTCCAGGCTGAGGATGAGGCT | | AITGLQAEDEADYYC | | | |
| | | GATTATTACTGCCAGTCCTATGACAGCGGCCTGAGTGGTTCGG | | QSYDSGLSGSVFGGG | | | |
| | | TATTCGGCGGAGGGACCAAGCTGACCGTCCTA | | TKLTVL | | | |
| ADI-81610 | 2947 | CAGTCTGTGCTGACTCAGCCGCCCTCAGTGTCTGGGGCCCCAG | 4839 | QSVLTQPPSVSGAPG | 5192 | 4 | 4 |
| | | GGCAGAGGGTCACCATCTCCTGCACTGGGAGCAACTCCAACAT | | QRVTISCTGSNSNIG | | | |
| | | CGGGGCAGGTTATGATGTGCACTGGTACCAGCAGCTTCCAGGA | | AGYDVHWYQQLPGTA | | | |
| | | ACAGCCCCCAAACTCCTCATCTATCGTAACACCAATCGGCCCT | | PKLLIYRNTNRPSGV | | | |
| | | CAGGGGTCCCTGACCGATTCTCTGGCTCCAAGTCTGGCGCCTC | | PDRFSGSKSGASASL | | | |
| | | AGCCTCCCTGGCCATCACTGGGCTCCAGGCTGAGGATGAGGCT | | AITGLQAEDEADYYC | | | |
| | | GATTATTACTGCCAGTCCTATGACAGCAGCCTGAGTGGTTCGG | | QSYDSSLSGSVFGGG | | | |
| | | TGTTCGGCGGAGGGACCAAGGTCACCGTCCTA | | TKVTVL | | | |
| ADI-81764 | 2944 | GACATCGCGATGACCCAGTCTCCATCCTCCCTGTCTGCATCTG | 4840 | DIAMTQSPSSLSASV | 5193 | 4 | 4 |
| | | TAGGAGACAGAGTCACCTTCACTTGCCGGGCAAGTCAGAGCAT | | GDRVTFTCRASQSIG | | | |
| | | TGGCATCTATTTAAATTGGTATCAGCAGAAACCAGGGAAAGCC | | IYLNWYQQKPGKAPK | | | |
| | | CCTAAACTCCTGATCTATGCTGCATCCACTTTGCAAAGTGGGG | | LLIYAASTLQSGVPS | | | |
| | | TCCCGTCAAGGTTCAGTGGCAGTGGATCTGGGACAGATTTCAC | | RFSGSGSGTDFTLTI | | | |
| | | TCTCACCATCAGCAGTCTGCAACCTGAAGATTTTGCAACTTAC | | SSLQPEDFATYYCQQ | | | |
| | | TACTGTCAACAGAGTTACACAACCCCCACGTGGACGTTCGGCC | | SYTTPTWTFGQGTKV | | | |
| | | AAGGGACCAAAGTGGATATCAAA | | DIK | | | |
| ADI-81868 | 2949 | CAGTCTGTCCTGACGCAGCCGCCCTCAGTGTCTGGGGCCCCAG | 4841 | QSVLTQPPSVSGAPG | 5194 | 4 | 3 |
| | | GGCAGAGGGTCATCATCTCCTGCACTGGGAGCGACTCCAACAT | | QRVIISCTGSDSNIG | | | |
| | | CGGGGCAGGTTATGATGTACACTGGTACCAGCAGCTTGCAGGA | | AGYDVHWYQQLAGTA | | | |
| | | ACAGCCCCCAAACTCCTCATCTATGGTAACAGTAATCGGCCCT | | PKLLIYGNSNRPSGV | | | |
| | | CAGGGGTCCCTGACCGATTCTCTGGCTCCAAGTCTGGCACCTC | | PDRFSGSKSGTSASL | | | |
| | | AGCCTCCCTGGCCATCACTGGGCTCCAGGCTGAGGATGAGGCT | | AITGLQAEDEADYYC | | | |
| | | GATTATTACTGCCAGTCCTTTGACAGCAGCCTGAGTGGTCCCA | | QSFDSSLSGPNYVFG | | | |
| | | ATTATGTCTTCGGAACTGGGACCAAGCTCACCGTCCTA | | TGTKLTVL | | | |
| ADI-81876 | 2939 | AATTTTATGCTGACTCAGCCCCACTCTGTGTCGGAGTCTCCGG | 4842 | NFMLTQPHSVSESPG | 5195 | 4 | 4 |
| | | GGAAGACGGTAACCATCTCCTGCACCCGCAGCAGTGGCGGCAT | | KTVTISCTRSSGGIV | | | |
| | | TGTCAACAACTATGTGCAGTGGTACCAGCAGCGCCCGGGCAGT | | NNYVQWYQQRPGSSP | | | |
| | | TCCCCCACCACTGTGATCTATGAGGATAACCAAAGACCCTCTG | | TTVIYEDNQRPSGVP | | | |
| | | GGGTCCCTGATCGGTTCTCTGGCTCCATCGACAGCTCCTCCAA | | DRFSGSIDSSSNSAS | | | |
| | | CTCTGCCTCCCTCACCATCTCTGGACTGAAGACTGAGGACGAG | | LTISGLKTEDEAVYY | | | |
| | | GCTGTCTACTACTGTCAGTCTTATGATACTAGCAATCATGGGG | | CQSYDTSNHGVFGGG | | | |
| | | TGTTCGGCGGCGGGACCAAGCTCACCGTCCTA | | TKLTVL | | | |
| ADI-81800 | 2937 | GAAATGACAATGACGCAGTCTCCAGCCACCCTGTCTGTGTCTC | 4843 | EMTMTQSPATLSVSP | 5196 | 4 | 4 |
| | | CAGGGGAAAGAGCCACCCTCTCCTGCAGGGCCAGTCAGAGTAT | | GERATLSCRASQSIS | | | |
| | | TAGCACCACCTTAGCCTGGTACCAGCAGAAACCTGGCCAGGCT | | TTLAWYQQKPGQAPR | | | |
| | | CCCAGACTCCTCATGTATGGTGCATCCACCAGGGCCACAGGTA | | LLMYGASTRATGIPA | | | |
| | | TCCCAGCCAGGTTCAGTGGCAGTGGGTCTGGGACAGAGTTCAC | | RFSGSGSGTEFTLTI | | | |
| | | TCTCACCATCAGCAGCCTGCAGTCTGAAGATTTTGCAGTTTAT | | SSLQSEDFAVYYCQQ | | | |
| | | TACTGTCAGCAGTATAATAATTGGACTCCGACGTTCGGCCAAG | | YNNWTPTFGQGTKVE | | | |
| | | GGACCAAGGTGGAGATCAAA | | IK | | | |
| ADI-81892 | 2936 | GAAATGACACTCACGCAGTCTCCATCCTCCCTGTCTGCATCTG | 4844 | EMTLTQSPSSLSASV | 5197 | 4 | 4 |
| | | TAGGAGACAGAGTCACCATCACTTGCCGGCCAAGTCAGAGCAT | | GDRVTITCRPSQSIS | | | |
| | | TAGCAGTTTTTTAAATTGGTATCAGCAGAAACCAGGGAGAGCC | | SFLNWYQQKPGRAPK | | | |
| | | CCTAAGCTCCTGATCTATGCTGCATCCAGTTTGCAAAGTGGGG | | LLIYAASSLQSGVPS | | | |
| | | TCCCATCAAGGTTCAGTGGCAGTGAATCTGGGACAGATTTCAC | | RFSGSESGTDFTLTI | | | |
| | | TCTCACCATCAGCAGTCTCCAACCTGAAGATTTTGCAACTTAC | | SSLQPEDFATYYCQQ | | | |
| | | TACTGTCAGCAGAATTACAATTCCCCCCTCACTTTCGGCGGTG | | NYNSPLTFGGGTKVE | | | |
| | | GGACCAAAGTGGAGATCAAA | | IK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81657 | IGLV8-61 | QSVVTQ | 3665 | GLSSGS | 3866 | WYRQTP | 4010 | NTKTRS | 4137 | GVPDRF | 4294 | MLYMGS | 4507 | FGGGTK |
| | | EPSFSV | | VSTSYY | | GQAPRT | | S | | SGSILG | | GIWV | | LTVL |
| | | SPGGTV | | PS | | LIY | | | | NKAALT | | | | |
| | | TLTC | | | | | | | | ITGAQA | | | | |
| | | | | | | | | | | DDESDY | | | | |
| | | | | | | | | | | HC | | | | |
| ADI-81776 | IGLV3-21 | SYELTQ | 1919 | GGNNIG | 2208 | WYQLKA | 4011 | DDSDRP | 2399 | GIPERF | 4295 | HVWDSS | 4508 | FGGGTK |
| | | PPSVSV | | SKSVH | | GQAPVL | | S | | SGSKSG | | SDHWV | | LTVV |
| | | APGQTA | | | | VVY | | | | NTATLI | | | | |
| | | RITC | | | | | | | | ISRVEA | | | | |
| | | | | | | | | | | GDEADY | | | | |
| | | | | | | | | | | YC | | | | |
| ADI-81810 | IGLV3-21 | SYELTQ | 3666 | GGNNIG | 2208 | WYQQKP | 2283 | DDRDRP | 4138 | GIPERF | 2674 | QVWDSS | 2892 | FGGGTK |
| | | PPSVSV | | SKSVH | | GQAPVL | | S | | SGSSSG | | SDLVI | | VTVL |
| | | APGQTA | | | | VVY | | | | NTATLT | | | | |
| | | RIPC | | | | | | | | LSRVEA | | | | |
| | | | | | | | | | | GDEADY | | | | |
| | | | | | | | | | | YC | | | | |
| ADI-81634 | IGLV3-25 | YYELTQ | 3667 | SGDTLP | 3867 | WYQQKP | 2304 | KDSERP | 2400 | GIPERF | 2546 | QSADSS | 4509 | FGGGTK |
| | | PPSVSV | | TQYAY | | GQAPVL | | S | | SGSTSG | | DIR | | LTVL |
| | | SPGQTA | | | | VIY | | | | TTVTLT | | | | |
| | | RITC | | | | | | | | ISGVQA | | | | |
| | | | | | | | | | | EDEADY | | | | |
| | | | | | | | | | | YC | | | | |
| ADI-81817 | IGKV1D-39 | DIPVTQ | 3642 | RASQSI | 2090 | WYQQKP | 2257 | AVSSLQ | 4123 | GVPSRF | 4293 | QQNYNS | 4470 | FGGGTK |
| | | SPSSLS | | SSFLN | | GKAPKL | | S | | SGSESG | | PLT | | VDIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81805 | IGLV6-57 | NFMLTQ | 1992 | TRSSGS | 2109 | WYQQRP | 2378 | EDNQRP | 2431 | GVPDRF | 2694 | QSYDSS | 2922 | FGGGTK |
| | | PHSVSE | | IASNYV | | GSSPST | | S | | SGSIDS | | LWV | | LTVL |
| | | SPGKTV | | Q | | VIY | | | | SSNSAS | | | | |
| | | TISC | | | | | | | | LTISGL | | | | |
| | | | | | | | | | | QTDDEA | | | | |
| | | | | | | | | | | DYYC | | | | |
| ADI-81576 | IGKV3-20 | ETTLTQ | 3668 | RASQSV | 3868 | WYQQKP | 2258 | GASSRA | 2464 | GIPDRF | 4296 | QQYRSS | 4510 | FGQGTK |
| | | SPGTLS | | SSSHLA | | GQAPRL | | T | | SGSGSG | | PWT | | VDIK |
| | | LSPGER | | | | LIY | | | | TDFTLT | | | | |
| | | ATFSC | | | | | | | | ISRLEP | | | | |
| | | | | | | | | | | EDVAVY | | | | |
| | | | | | | | | | | YC | | | | |
| ADI-81665 | IGKV3-20 | ETTLTQ | 3669 | RASQSV | 2230 | WYQQKP | 3996 | GASSRA | 4139 | GIPDRF | 2616 | QQYVTS | 4511 | FGQGTK |
| | | SPGTLS | | SSSYLA | | GQAPRL | | P | | SGSGSG | | PT | | VEIK |
| | | LSPGER | | | | LIS | | | | TDFTLT | | | | |
| | | VTLSC | | | | | | | | ISRLEP | | | | |
| | | | | | | | | | | EDFAVY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81657 | 2939 | CAGTCTGTGGTGACTCAGGAGCCATCGTTCTCAGTGTCCCCTG | 4845 | QSVVTQEPSFSVSPG | 5198 | 4 | 4 |
| | | GAGGGACAGTCACACTCACTTGTGGCTTGAGCTCTGGCTCAGT | | GTVTLTCGLSSGSVS | | | |
| | | CTCTACTAGTTACTACCCCAGCTGGTACCGGCAGACCCCAGGC | | TSYYPSWYRQTPGQA | | | |
| | | CAGGCTCCACGCACGCTCATCTACAACACAAAGACTCGCTCTT | | PRTLIYNTKTRSSGV | | | |
| | | CTGGGGTCCCTGATCGCTTCTCTGGCTCCATCCTTGGGAACAA | | PDRFSGSILGNKAAL | | | |
| | | AGCTGCCCTCACCATCACGGGGGCCCAGGCAGATGATGAATCT | | TITGAQADDESDYHC | | | |
| | | GATTATCACTGTATGCTATATATGGGTAGTGGCATTTGGGTGT | | MLYMGSGIWVFGGGT | | | |
| | | TCGGCGGAGGGACCAAGCTCACCGTCCTA | | KLTVL | | | |
| ADI-81776 | 4600 | TCGTATGAGCTGACTCAGCCACCCTCGGTGTCAGTGGCCCCAG | 4846 | SYELTQPPSVSVAPG | 5199 | 4 | 4 |
| | | GACAGACGGCCAGGATTACCTGTGGGGGAAACAACATTGGAAG | | QTARITCGGNNIGSK | | | |
| | | TAAAAGTGTGCACTGGTACCAGCTGAAGGCAGGCCAGGCCCCT | | SVHWYQLKAGQAPVL | | | |
| | | GTGCTGGTCGTCTATGATGATAGCGACCGGCCCTCAGGGATCC | | VVYDDSDRPSGIPER | | | |
| | | CGGAGCGATTCTCTGGCTCCAAGTCTGGGAACACGGCCACCCT | | FSGSKSGNTATLIIS | | | |
| | | GATCATCAGCAGGGTCGAAGCCGGGGATGAGGCCGACTATTAC | | RVEAGDEADYYCHVW | | | |
| | | TGTCACGTGTGGGATAGTAGTAGTGATCATTGGGTATTCGGCG | | DSSSDHWVFGGGTKL | | | |
| | | GAGGGACCAAGCTCACCGTCGTA | | TWV | | | |
| ADI-81810 | 2947 | TCGTATGAGCTGACTCAGCCACCCTCGGTGTCAGTGGCCCCAG | 4847 | SYELTQPPSVSVAPG | 5200 | 4 | 4 |
| | | GACAGACGGCCAGGATTCCCTGTGGGGGAAACAACATTGGAAG | | QTARIPCGGNNIGSK | | | |
| | | TAAAAGTGTGCACTGGTACCAGCAGAAGCCAGGCCAGGCCCCT | | SVHWYQQKPGQAPVL | | | |
| | | GTGCTGGTCGTCTATGATGATCGCGACCGGCCCTCAGGGATCC | | VVYDDRDRPSGIPER | | | |
| | | CTGAGCGATTCTCTGGCTCCAGCTCTGGGAACACGGCCACCCT | | FSGSSSGNTATLTLS | | | |
| | | GACCCTCAGCAGGGTCGAAGCCGGGGATGAGGCCGACTATTAC | | RVEAGDEADYYCQVW | | | |
| | | TGTCAGGTGTGGGATAGTAGTAGTGATCTTGTGATATTCGGCG | | DSSSDLVIFGGGTKV | | | |
| | | GAGGGACCAAGGTCACCGTCCTA | | TVL | | | |
| ADI-81634 | 2939 | TATTATGAGCTGACTCAGCCACCCTCGGTGTCGGTGTCCCCAG | 4848 | YYELTQPPSVSVSPG | 5201 | 4 | 3 |
| | | GACAGACGGCCAGGATCACCTGCTCTGGAGATACATTGCCAAC | | QTARITCSGDTLPTQ | | | |
| | | ACAATATGCTTATTGGTACCAGCAGAAGCCAGGCCAGGCCCCT | | YAYWYQQKPGQAPVL | | | |
| | | GTGTTGGTGATATATAAAGACAGTGAGAGGCCCTCAGGGATCC | | VIYKDSERPSGIPER | | | |
| | | CTGAGCGATTCTCTGGCTCCACCTCAGGGACAACAGTCACGTT | | FSGSTSGTTVTLTIS | | | |
| | | GACCATCAGTGGAGTCCAGGCAGAAGACGAGGCTGACTATTAC | | GVQAEDEADYYCQSA | | | |
| | | TGTCAATCAGCAGACAGCAGTGATATCCGATTCGGCGGAGGGA | | DSSDIRFGGGTKLTV | | | |
| | | CCAAGCTCACCGTCCTA | | L | | | |
| ADI-81817 | 2938 | GACATCCCGGTGACGCAGTCTCCGTCCTCCCTGTCTGCATCTG | 4849 | DIPVTQSPSSLSASV | 5202 | 4 | 3 |
| | | TAGGAGACAGAGTCACCATCACTTGCCGGGCAAGTCAGAGCAT | | GDRVTITCRASQSIS | | | |
| | | TAGCAGCTTTTTAAACTGGTATCAGCAGAAACCAGGGAAAGCC | | SFLNWYQQKPGKAPK | | | |
| | | CCTAAGCTCCTGATCTATGCTGTGTCCAGTTTGCAAAGTGGGG | | LLIYAVSSLQSGVPS | | | |
| | | TCCCATCAAGGTTCAGTGGCAGTGAATCTGGGACAGATTTCAC | | RFSGSESGTDFTLTI | | | |
| | | TCTCACCATCAGCAGTCTGCAACCTGAAGATTTTGCAACTTAC | | SSLQPEDFATYYCQQ | | | |
| | | TACTGTCAACAGAATTACAATTCCCCCCTCACTTTCGGCGGGG | | NYNSPLTFGGGTKVD | | | |
| | | GGACCAAAGTGGATATCAAA | | IK | | | |
| ADI-81805 | 2939 | AATTTTATGCTGACTCAGCCCCACTCTGTGTCGGAGTCTCCGG | 4850 | NFMLTQPHSVSESPG | 5203 | 3 | 3 |
| | | GGAAGACAGTAACCATCTCCTGCACCCGCAGCAGTGGCAGCAT | | KTVTISCTRSSGSIA | | | |
| | | TGCCAGCAACTATGTGCAGTGGTACCAGCAGCGCCCGGGCAGT | | SNYVQWYQQRPGSSP | | | |
| | | TCACCTTCCACTGTTATCTATGAGGATAACCAAAGACCCTCTG | | STVIYEDNQRPSGVP | | | |
| | | GGGTCCCTGATCGGTTCTCTGGCTCCATCGACAGCTCCTCCAA | | DRFSGSIDSSSNSAS | | | |
| | | CTCTGCCTCCCTCACCATCTCTGGACTGCAGACTGACGACGAG | | LTISGLQTDDEADYY | | | |
| | | GCTGACTACTACTGTCAGTCTTATGATAGCAGCCTTTGGGTGT | | CQSYDSSLWVFGGGT | | | |
| | | TCGGCGGAGGGACCAAGCTCACCGTCCTA | | KLTVL | | | |
| ADI-81576 | 2944 | GAAACGACACTCACGCAGTCTCCAGGCACCCTGTCTTTGTCTC | 4851 | ETTLTQSPGTLSLSP | 5204 | 3 | 3 |
| | | CAGGGGAAAGAGCCACCTTCTCCTGCAGGGCCAGTCAGAGTGT | | GERATFSCRASQSVS | | | |
| | | TAGCAGCAGCCACTTAGCCTGGTACCAGCAGAAACCTGGCCAG | | SSHLAWYQQKPGQAP | | | |
| | | GCTCCCAGGCTCCTCATCTATGGTGCATCCAGCAGGGCCACTG | | RLLIYGASSRATGIP | | | |
| | | GCATCCCAGACAGGTTCAGTGGGAGTGGGTCAGGGACAGACTT | | DRFSGSGSGTDFTLT | | | |
| | | CACTCTCACCATCAGCAGACTGGAGCCTGAAGATGTTGCAGTG | | ISRLEPEDVAVYYCQ | | | |
| | | TATTACTGTCAGCAGTATCGTAGCTCACCGTGGACGTTCGGCC | | QYRSSPWTFGQGTKV | | | |
| | | AAGGGACCAAAGTGGATATCAAA | | DIK | | | |
| ADI-81665 | 2937 | GAAACGACACTCACCCAGTCTCCAGGCACCCTATCTTTGTCTC | 4852 | ETTLTQSPGTLSLSP | 5205 | 3 | 3 |
| | | CAGGTGAAAGAGTCACCCTCTCCTGCAGGGCCAGTCAGAGTGT | | GERVTLSCRASQSVS | | | |
| | | TAGCAGCAGCTACTTAGCCTGGTACCAGCAGAAACCTGGCCAG | | SSYLAWYQQKPGQAP | | | |
| | | GCTCCCAGGCTCCTCATCTCTGGTGCATCCAGCAGGGCCCCTG | | RLLISGASSRAPGIP | | | |
| | | GCATTCCAGACAGGTTCAGTGGCAGTGGGTCTGGGACAGACTT | | DRFSGSGSGTDFTLT | | | |
| | | CACTCTCACCATCAGCAGACTGGAGCCTGAAGATTTTGCAGTG | | ISRLEPEDFAVYYCQ | | | |
| | | TATTACTGTCAGCAGTATGTTACCTCACCCACTTTTGGCCAGG | | QYVTSPTFGQGTKVE | | | |
| | | GGACCAAGGTGGAGATCAAA | | IK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81714 | IGKV1D-39 | EIPMTQ | 1912 | RASQSI | 2066 | WYQQKP | 2257 | AASSLH | 2426 | GVPSRF | 2552 | QQSYST | 4435 | FGGGTK |
| | | SPSSLS | | STYLN | | GKAPKL | | S | | SGSGSG | | PPLT | | VEIK |
| | | ASVGDR | | | | LIY | | | | TDFTLT | | | | |
| | | VTITC | | | | | | | | ISSLQP | | | | |
| | | | | | | | | | | EDLATY | | | | |
| | | | | | | | | | | YC | | | | |
| ADI-81683 | IGKV3-11 | EMALTQ | 3670 | RASQSV | 2119 | WYQQKP | 2258 | GASNRA | 4107 | GIPARF | 2607 | QQRTNW | 4512 | FGQGTK |
| | | SPATLS | | STYLA | | GQAPRL | | T | | SGSGSG | | PRMYT | | VEIK |
| | | LSPGEG | | | | LIY | | | | TDFTLT | | | | |
| | | ATLSC | | | | | | | | ISSLEP | | | | |
| | | | | | | | | | | EDFAVY | | | | |
| | | | | | | | | | | YC | | | | |
| ADI-81820 | IGLV3-21 | SYELTQ | 1919 | GGNEIG | 3869 | WYQQKP | 2358 | DDSDRP | 2399 | GIPERF | 2545 | QVWDSI | 4513 | FGGGTK |
| | | PPSVSV | | SKSVH | | GQAPVL | | S | | SGSNSG | | SHHFNW | | LTVL |
| | | APGQTA | | | | VVN | | | | NTATLT | | V | | |
| | | RITC | | | | | | | | ISRVEA | | | | |
| | | | | | | | | | | GDEADY | | | | |
| | | | | | | | | | | YC | | | | |
| ADI-81569 | IGKV1-33 | EIQLTQ | 1917 | QASQDI | 3870 | WYQQKP | 2257 | DASNFE | 4140 | GVPSRF | 4297 | QQFDSL | 4514 | FGGGTK |
| | | SPSSLS | | TNYLN | | GKAPKL | | T | | SGSGSG | | PLT | | VEIK |
| | | ASVGDR | | | | LIY | | | | TDFTFT | | | | |
| | | VTITC | | | | | | | | ISSLQP | | | | |
| | | | | | | | | | | EDIAIY | | | | |
| | | | | | | | | | | YC | | | | |
| ADI-81873 | IGKV1D-39 | EIRMTQ | 3671 | RASQSI | 2050 | WYQQKP | 2257 | AASTLQ | 2392 | GVPSRF | 4298 | QQSYIM | 4515 | FGQGTK |
| | | SPSSLS | | SSYLN | | GKAPKL | | S | | SGSGFG | | PPWT | | VEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | HC | | | | |
| ADI-81534 | IGKV1D-39 | DIQMTQ | 1878 | RASQRI | 2135 | WYQQKV | 4012 | AASSLQ | 2391 | GVPSRF | 2534 | QQSYVM | 4516 | FGQGTK |
| | | SPSSLS | | SSYLN | | GKAPKL | | S | | SGSGSG | | PPWT | | VEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81671 | IGLV1-44 | QSVLTQ | 3566 | SGSSSN | 2162 | WYQQLP | 3951 | SNNQRP | 2463 | GVPDRF | 2615 | ATWDDS | 2815 | FGGGTK |
| | | PPSASG | | IGSNSV | | RTAPKL | | S | | SGSKSG | | LTGPV | | VTVL |
| | | TPGQRV | | N | | LVY | | | | TSASLA | | | | |
| | | TISC | | | | | | | | ISGLQS | | | | |
| | | | | | | | | | | EDEADY | | | | |
| | | | | | | | | | | YC | | | | |
| ADI-81749 | IGKV3-11 | ETTLTQ | 3633 | RASQSV | 3871 | WYQQKP | 2258 | DASNRA | 2448 | GIPGRF | 4299 | QLRSNW | 4517 | FGGGTK |
| | | SPATLS | | SNYLA | | GQAPRL | | T | | SGSGSG | | PPRLS | | VEIK |
| | | LSPGER | | | | LIY | | | | TDFTLT | | | | |
| | | ATLSC | | | | | | | | ISGLEP | | | | |
| | | | | | | | | | | EDFAVY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81714 | 2936 | GAAATCCCAATGACCCAGTCTCCATCCTCCCTGTCTGCATCTG | 4853 | EIPMTQSPSSLSASV | 5206 | 3 | 3 |
| | | TAGGAGACAGAGTCACCATCACTTGCCGGGCAAGTCAGAGCAT | | GDRVTITCRASQSIS | | | |
| | | TAGCACCTATTTAAATTGGTATCAGCAGAAACCAGGGAAAGCC | | TYLNWYQQKPGKAPK | | | |
| | | CCTAAACTCCTGATCTATGCTGCTTCCAGTTTGCATAGTGGGG | | LLIYAASSLHSGVPS | | | |
| | | TCCCATCAAGGTTCAGTGGCAGTGGATCAGGGACAGATTTCAC | | RFSGSGSGTDFTLTI | | | |
| | | TCTCACCATCAGCAGTCTGCAACCTGAAGATCTTGCAACTTAC | | SSLQPEDLATYYCQQ | | | |
| | | TACTGTCAACAGAGTTACAGTACCCCTCCGCTCACTTTCGGCG | | SYSTPPLTFGGGTKV | | | |
| | | GAGGGACCAAGGTGGAGATCAAA | | EIK | | | |
| ADI-81683 | 2937 | GAAATGGCACTCACGCAGTCTCCAGCCACCCTGTCTTTGTCTC | 4854 | EMALTQSPATLSLSP | 5207 | 3 | 3 |
| | | CAGGGGAAGGAGCCACCCTCTCCTGCAGGGCCAGTCAGAGTGT | | GEGATLSCRASQSVS | | | |
| | | TAGCACCTACTTAGCCTGGTACCAACAGAAACCTGGCCAGGCT | | TYLAWYQQKPGQAPR | | | |
| | | CCCAGGCTCCTCATCTATGGTGCATCCAACAGGGCCACTGGCA | | LLIYGASNRATGIPA | | | |
| | | TCCCAGCCAGGTTCAGTGGCAGTGGGTCTGGGACAGACTTCAC | | RFSGSGSGTDFTLTI | | | |
| | | TCTCACCATCAGCAGCCTAGAGCCTGAAGATTTTGCAGTTTAT | | SSLEPEDFAVYYCQQ | | | |
| | | TACTGTCAGCAGCGTACCAACTGGCCTCGGATGTACACTTTTG | | RTNWPRMYTFGQGTK | | | |
| | | GCCAGGGGACCAAGGTGGAGATCAAA | | VEIK | | | |
| ADI-81820 | 2939 | TCGTATGAGCTGACGCAGCCACCCTCGGTGTCAGTGGCCCCAG | 4855 | SYELTQPPSVSVAPG | 5208 | 3 | 2 |
| | | GACAGACGGCCAGGATTACCTGTGGGGGAAATGAGATTGGAAG | | QTARITCGGNEIGSK | | | |
| | | TAAAAGTGTGCACTGGTACCAGCAGAAGCCAGGCCAGGCCCCT | | SVHWYQQKPGQAPVL | | | |
| | | GTGCTGGTCGTCAATGATGATAGCGACCGGCCCTCAGGGATCC | | VVNDDSDRPSGIPER | | | |
| | | CTGAGCGATTCTCTGGCTCCAACTCTGGAAACACGGCCACCCT | | FSGSNSGNTATLTIS | | | |
| | | GACCATCAGCAGGGTCGAAGCCGGGGATGAGGCCGACTATTAC | | RVEAGDEADYYCQVW | | | |
| | | TGTCAGGTGTGGGATAGTATTAGTCATCATTTTAATTGGGTGT | | DSISHHFNWVFGGGT | | | |
| | | TCGGCGGAGGGACCAAGCTCACCGTCCTA | | KLTVL | | | |
| ADI-81569 | 2936 | GAAATCCAGTTGACCCAGTCTCCATCCTCCCTGTCTGCATCTG | 4856 | EIQLTQSPSSLSASV | 5209 | 3 | 3 |
| | | TAGGAGACAGAGTCACCATCACTTGCCAGGCGAGTCAGGACAT | | GDRVTITCQASQDIT | | | |
| | | TACCAACTATTTAAATTGGTATCAGCAGAAACCAGGGAAAGCC | | NYLNWYQQKPGKAPK | | | |
| | | CCTAAGCTCCTGATCTACGATGCATCCAATTTCGAAACAGGGG | | LLIYDASNFETGVPS | | | |
| | | TCCCATCAAGGTTCAGTGGAAGTGGATCTGGGACAGATTTTAC | | RFSGSGSGTDFTFTI | | | |
| | | TTTCACCATCAGCAGCCTGCAGCCTGAAGATATTGCAATATAT | | SSLQPEDIAIYYCQQ | | | |
| | | TACTGTCAACAGTTTGATAGTCTCCCCCTCACTTTCGGCGGAG | | FDSLPLTFGGGTKVE | | | |
| | | GGACCAAGGTGGAAATCAAA | | IK | | | |
| ADI-81873 | 2937 | GAAATCCGGATGACCCAGTCTCCATCCTCCCTGTCTGCATCTG | 4857 | EIRMTQSPSSLSASV | 5210 | 3 | 3 |
| | | TAGGAGACAGAGTCACCATCACTTGCCGGGCAAGTCAGAGCAT | | GDRVTITCRASQSIS | | | |
| | | TAGCAGCTATTTAAATTGGTATCAGCAGAAACCAGGGAAAGCC | | SYLNWYQQKPGKAPK | | | |
| | | CCTAAGCTCCTGATTTATGCTGCATCCACTTTGCAAAGTGGGG | | LLIYAASTLQSGVPS | | | |
| | | TCCCATCAAGATTCAGTGGCAGTGGATTTGGGACAGATTTCAC | | RFSGSGFGTDFTLTI | | | |
| | | TCTCACCATCAGCAGTCTGCAACCTGAAGATTTTGCAACTTAC | | SSLQPEDFATYHCQQ | | | |
| | | CACTGTCAACAGAGTTACATTATGCCACCCTGGACGTTCGGCC | | SYIMPPWTFGQGTKV | | | |
| | | AAGGGACCAAGGTGGAAATCAAA | | EIK | | | |
| ADI-81534 | 2937 | GACATCCAGATGACCCAGTCTCCATCCTCCCTGTCTGCATCTG | 4858 | DIQMTQSPSSLSASV | 5211 | 3 | 2 |
| | | TAGGAGACAGAGTCACCATCACTTGTCGGGCAAGTCAGAGGAT | | GDRVTITCRASQRIS | | | |
| | | TAGCAGTTATTTAAATTGGTATCAGCAAAAAGTAGGGAAGGCC | | SYLNWYQQKVGKAPK | | | |
| | | CCTAAGCTCCTGATCTATGCTGCATCCAGTTTGCAAAGTGGGG | | LLIYAASSLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAACCTGAAGATTTTGCCACTTAC | | SSLQPEDFATYYCQQ | | | |
| | | TACTGTCAACAGAGTTACGTTATGCCCCCGTGGACGTTCGGCC | | SYVMPPWTFGQGTKV | | | |
| | | AAGGGACCAAGGTGGAAATCAAA | | EIK | | | |
| ADI-81671 | 2947 | CAGTCTGTGCTGACTCAGCCCCCCTCAGCGTCTGGGACCCCCG | 4859 | QSVLTQPPSASGTPG | 5212 | 3 | 3 |
| | | GGCAGAGGGTCACCATCTCTTGTTCTGGAAGCAGCTCCAACAT | | QRVTISCSGSSSNIG | | | |
| | | CGGAAGTAATTCTGTAAACTGGTACCAGCAACTCCCAAGAACG | | SNSVNWYQQLPRTAP | | | |
| | | GCCCCCAAACTCCTCGTCTATAGTAATAATCAGCGGCCCTCAG | | KLLVYSNNQRPSGVP | | | |
| | | GGGTCCCTGACCGATTCTCTGGCTCCAAGTCTGGCACCTCAGC | | DRFSGSKSGTSASLA | | | |
| | | CTCCCTGGCCATCAGTGGGCTCCAGTCTGAGGATGAGGCTGAT | | ISGLQSEDEADYYCA | | | |
| | | TATTACTGCGCAACATGGGATGACAGCCTGACTGGTCCGGTGT | | TWDDSLTGPVFGGGT | | | |
| | | TCGGCGGAGGGACCAAGGTCACCGTCCTA | | KVTVL | | | |
| ADI-81749 | 2936 | GAAACGACACTCACGCAGTCTCCAGCCACCCTGTCTTTGTCTC | 4860 | ETTLTQSPATLSLSP | 5213 | 3 | 3 |
| | | CAGGGGAAAGAGCCACCCTCTCCTGCAGGGCCAGTCAGAGTGT | | GERATLSCRASQSVS | | | |
| | | TAGCAACTACTTAGCCTGGTACCAACAGAAACCTGGCCAGGCT | | NYLAWYQQKPGQAPR | | | |
| | | CCCAGGCTCCTCATCTATGATGCTTCCAACAGGGCCACTGGCA | | LLIYDASNRATGIPG | | | |
| | | TCCCAGGCAGGTTCAGTGGCAGTGGGTCTGGGACAGACTTCAC | | RFSGSGSGTDFTLTI | | | |
| | | TCTCACCATCAGCGGCCTAGAGCCTGAAGACTTTGCAGTTTAT | | SGLEPEDFAVYYCQL | | | |
| | | TACTGTCAGCTGCGTAGCAACTGGCCTCCGCGCCTCTCTTTCG | | RSNWPPRLSFGGGTK | | | |
| | | GCGGAGGGACCAAGGTGGAAATCAAA | | VEIK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81668 | IGKV4-1 | DIVMTQ | 2046 | KSSQSV | 3872 | WYQQKP | 4013 | WASTRE | 2422 | GVPDRF | 2619 | QQYYST | 4518 | FGQGTK |
| | | SPDSLA | | LYSSNS | | GQPPKL | | S | | SGSGSG | | PLT | | VDIK |
| | | VSLGER | | QNYLA | | LLY | | | | TDFTLT | | | | |
| | | ATINC | | | | | | | | ISSLQA | | | | |
| | | | | | | | | | | EDVAVY | | | | |
| | | | | | | | | | | YC | | | | |
| ADI-81771 | IGKV3-20 | EMTLTQ | 1963 | RASQSV | 3873 | WYQQKA | 4014 | GASSRA | 2464 | GIPDRF | 4300 | QQYGYA | 4519 | FGQGTK |
| | | SPGTLS | | SSTYLA | | GQAPRL | | T | | SGSGAG | | PWT | | VEIK |
| | | LSPGER | | | | LIY | | | | TDFTLT | | | | |
| | | ATLSC | | | | | | | | ISRLEP | | | | |
| | | | | | | | | | | EDFAVY | | | | |
| | | | | | | | | | | YC | | | | |
| ADI-81865 | IGKV1-5 | EIVLTQ | 3672 | RASQSI | 2190 | WYQQKP | 2316 | KATNLE | 4141 | GVPSRF | 2532 | QQYNSY | 2798 | FGQGTK |
| | | SPSTLS | | SSWLA | | GKAPKV | | S | | SGSGSG | | YT | | VEIK |
| | | ASVGDR | | | | LIY | | | | TEFTLT | | | | |
| | | VTITC | | | | | | | | ISSLQP | | | | |
| | | | | | | | | | | DDFATY | | | | |
| | | | | | | | | | | YC | | | | |
| ADI-81670 | IGKV1-5 | EIVLTQ | 3672 | RASQSI | 2190 | WYQQKP | 2263 | KASSLE | 4142 | GVPSRF | 4301 | QQYNSF | 4520 | FGQGTK |
| | | SPSTLS | | SSWLA | | GKAPNL | | N | | SGSGSG | | PYT | | VEIK |
| | | ASVGDR | | | | LIY | | | | TEFTLT | | | | |
| | | VTITC | | | | | | | | ITSLQP | | | | |
| | | | | | | | | | | DDFATY | | | | |
| | | | | | | | | | | YC | | | | |
| ADI-81702 | IGKV1-5 | EMTMTQ | 3673 | RASQSI | 3812 | WYQQKP | 2275 | KASSLE | 2386 | GVPSRF | 2532 | QQYSSY | 4521 | FGQGTK |
| | | SPSTVS | | SNWLA | | GRAPKL | | S | | SGSGSG | | PRT | | VEIK |
| | | ASVGDR | | | | LIY | | | | TEFTLT | | | | |
| | | VTITC | | | | | | | | ISSLQP | | | | |
| | | | | | | | | | | DDFATY | | | | |
| | | | | | | | | | | YC | | | | |
| ADI-81754 | IGKV1-33 | EIQMTQ | 1881 | QASQDI | 3874 | WYQQKP | 2257 | DASNLE | 2416 | GVPSRF | 4269 | QQYDNL | 4522 | FGQGTK |
| | | SPSSLS | | SNSLH | | GKAPKL | | T | | SGSGSG | | PSYT | | VEIK |
| | | ASVGDR | | | | LIY | | | | TDFTLT | | | | |
| | | VTITC | | | | | | | | ISSLQP | | | | |
| | | | | | | | | | | EDIATY | | | | |
| | | | | | | | | | | YC | | | | |
| ADI-81855 | IGKV1-NL1 | EIAMTQ | 3674 | RASQGI | 2062 | WYQQKP | 2259 | AASTLE | 2398 | GVPSRF | 4283 | QQYYSN | 4483 | FGQGTK |
| | | SPSSLS | | SNSLA | | GKAPKL | | S | | SGSGSG | | LGRT | | VDIK |
| | | ASVGDR | | | | LLY | | | | TDFTLT | | | | |
| | | VTITC | | | | | | | | ISSLQP | | | | |
| | | | | | | | | | | EDFSTY | | | | |
| | | | | | | | | | | YC | | | | |
| ADI-81573 | IGLV1-44 | SYELTQ | 1962 | SGSSSN | 3875 | WYQQLP | 2314 | NNNQRP | 4061 | GVPDRF | 4302 | AAWDDS | 4413 | FGTGTK |
| | | PPSASG | | IGSNTL | | GTAPKL | | S | | SGSKSG | | LNGYV | | LTVL |
| | | TPGQRV | | N | | LIY | | | | TSASLA | | | | |
| | | TISC | | | | | | | | IRGLQS | | | | |
| | | | | | | | | | | EDEADY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81668 | 2944 | GACATCGTGATGACCCAGTCTCCAGACTCCCTGGCTGTGTCTC | 4861 | DIVMTQSPDSLAVSL | 5214 | 3 | 3 |
| | | TGGGCGAGAGGGCCACCATCAACTGCAAGTCCAGCCAGAGTGT | | GERATINCKSSQSVL | | | |
| | | TTTATACAGCTCCAACAGTCAGAATTACTTAGCTTGGTACCAG | | YSSNSQNYLAWYQQK | | | |
| | | CAGAAACCAGGACAGCCTCCTAAACTGCTCCTTTACTGGGCAT | | PGQPPKLLLYWASTR | | | |
| | | CTACCCGGGAATCCGGGGTCCCTGACCGATTCAGTGGCAGCGG | | ESGVPDRFSGSGSGT | | | |
| | | GTCTGGGACAGATTTCACTCTCACCATCAGCAGCCTGCAGGCT | | DFTLTISSLQAEDVA | | | |
| | | GAAGATGTGGCGGTTTATTACTGTCAGCAATATTATAGTACTC | | VYYCQQYYSTPLTFG | | | |
| | | CTCTGACGTTCGGCCAAGGGACCAAAGTGGATATCAAA | | QGTKVDIK | | | |
| ADI-81771 | 2937 | GAAATGACACTGACGCAGTCTCCAGGCACCCTGTCTTTGTCTC | 4862 | EMTLTQSPGTLSLSP | 5215 | 3 | 3 |
| | | CAGGGGAAAGAGCCACCCTCTCCTGCAGGGCCAGTCAGAGTGT | | GERATLSCRASQSVS | | | |
| | | TAGTAGCACCTACTTAGCCTGGTACCAGCAGAAAGCTGGCCAG | | STYLAWYQQKAGQAP | | | |
| | | GCTCCCAGGCTCCTCATCTATGGTGCATCCAGCAGGGCCACTG | | RLLIYGASSRATGIP | | | |
| | | GGATCCCAGACAGGTTCAGTGGCAGTGGGGCTGGGACAGACTT | | DRFSGSGAGTDFTLT | | | |
| | | CACTCTCACGATCAGTAGACTGGAGCCTGAAGATTTTGCAGTG | | ISRLEPEDFAVYYCQ | | | |
| | | TATTACTGTCAGCAATATGGTTACGCACCGTGGACGTTCGGCC | | QYGYAPWTFGQGTKV | | | |
| | | AAGGGACCAAGGTGGAAATCAAA | | EIK | | | |
| ADI-81865 | 2937 | GAAATCGTGTTGACCCAGTCTCCTTCCACCCTGTCTGCATCTG | 4863 | EIVLTQSPSTLSASV | 5216 | 3 | 3 |
| | | TAGGAGACAGAGTCACCATCACTTGCCGGGCCAGTCAGAGTAT | | GDRVTITCRASQSIS | | | |
| | | TAGTAGCTGGTTGGCCTGGTATCAGCAGAAACCAGGGAAAGCC | | SWLAWYQQKPGKAPK | | | |
| | | CCTAAGGTCCTGATCTATAAGGCAACTAATTTAGAAAGTGGGG | | VLIYKATNLESGVPS | | | |
| | | TCCCATCAAGGTTCAGCGGCAGTGGATCTGGGACAGAATTCAC | | RFSGSGSGTEFTLTI | | | |
| | | TCTCACCATCAGCAGCCTGCAGCCTGATGATTTTGCAACCTAT | | SSLQPDDFATYYCQQ | | | |
| | | TACTGCCAACAGTATAATAGTTATTACACTTTTGGCCAGGGGA | | YNSYYTFGQGTKVEI | | | |
| | | CCAAAGTGGAAATCAAA | | K | | | |
| ADI-81670 | 2937 | GAAATCGTGTTGACCCAGTCTCCCTCCACCCTGTCTGCATCTG | 4864 | EIVLTQSPSTLSASV | 5217 | 3 | 3 |
| | | TAGGAGACAGAGTCACCATCACTTGCCGGGCCAGTCAGAGTAT | | GDRVTITCRASQSIS | | | |
| | | TAGTAGTTGGTTGGCCTGGTATCAGCAGAAACCTGGGAAGGCC | | SWLAWYQQKPGKAPN | | | |
| | | CCTAACCTCCTGATCTATAAGGCGTCTAGTTTAGAAAATGGGG | | LLIYKASSLENGVPS | | | |
| | | TCCCATCAAGGTTCAGCGGCAGTGGATCTGGGACAGAATTCAC | | RFSGSGSGTEFTLTI | | | |
| | | TCTCACCATCACCAGCCTGCAGCCTGATGATTTTGCAACTTAT | | TSLQPDDFATYYCQQ | | | |
| | | TACTGCCAACAGTATAATAGTTTTCCGTACACTTTTGGCCAGG | | YNSFPYTFGQGTKVE | | | |
| | | GGACCAAGGTGGAAATCAAA | | IK | | | |
| ADI-81702 | 2937 | GAAATGACAATGACGCAGTCTCCTTCCACCGTGTCTGCATCTG | 4865 | EMTMTQSPSTVSASV | 5218 | 3 | 3 |
| | | TAGGAGACAGAGTCACCATCACTTGCCGGGCCAGTCAGAGTAT | | GDRVTITCRASQSIS | | | |
| | | TAGTAACTGGTTGGCCTGGTATCAACAAAAACCAGGGAGAGCC | | NWLAWYQQKPGRAPK | | | |
| | | CCTAAACTCCTGATCTATAAGGCGTCTAGTTTAGAAAGTGGGG | | LLIYKASSLESGVPS | | | |
| | | TCCCATCAAGGTTCAGTGGCAGTGGATCTGGGACAGAATTCAC | | RFSGSGSGTEFTLTI | | | |
| | | TCTCACCATCAGCAGCCTGCAGCCTGATGATTTTGCAACTTAT | | SSLQPDDFATYYCQQ | | | |
| | | TACTGCCAACAGTATAGCAGTTATCCAAGGACGTTCGGCCAAG | | YSSYPRTFGQGTKVE | | | |
| | | GGACCAAGGTGGAGATCAAA | | IK | | | |
| ADI-81754 | 2937 | GAAATCCAAATGACCCAGTCTCCTTCCTCCCTGTCTGCATCTG | 4866 | EIQMTQSPSSLSASV | 5219 | 3 | 3 |
| | | TAGGAGACAGAGTCACCATCACTTGCCAGGCGAGTCAGGACAT | | GDRVTITCQASQDIS | | | |
| | | TAGCAACTCTTTACATTGGTATCAGCAGAAACCAGGGAAAGCC | | NSLHWYQQKPGKAPK | | | |
| | | CCTAAGCTCCTGATCTACGATGCATCCAATTTGGAAACAGGTG | | LLIYDASNLETGVPS | | | |
| | | TCCCATCAAGGTTCAGTGGAAGTGGATCTGGGACAGATTTTAC | | RFSGSGSGTDFTLTI | | | |
| | | TCTCACCATCAGCAGCCTGCAGCCTGAAGATATTGCAACATAT | | SSLQPEDIATYYCQQ | | | |
| | | TACTGTCAACAGTATGATAATCTCCCTTCGTATACTTTTGGCC | | YDNLPSYTFGQGTKV | | | |
| | | AGGGGACCAAGGTGGAGATCAAA | | EIK | | | |
| ADI-81855 | 2944 | GAAATCGCAATGACCCAGTCTCCATCCTCCCTGTCTGCATCTG | 4867 | EIAMTQSPSSLSASV | 5220 | 3 | 3 |
| | | TAGGAGACAGAGTCACCATCACTTGCCGGGCGAGTCAGGGTAT | | GDRVTITCRASQGIS | | | |
| | | TAGCAATTCTTTAGCCTGGTATCAGCAGAAACCAGGGAAAGCC | | NSLAWYQQKPGKAPK | | | |
| | | CCTAAGCTCCTGCTCTATGCTGCATCCACATTGGAAAGTGGGG | | LLLYAASTLESGVPS | | | |
| | | TCCCTTCCAGGTTCAGTGGCAGTGGATCTGGGACGGATTTCAC | | RFSGSGSGTDFTLTI | | | |
| | | TCTCACCATCAGCAGCCTGCAGCCTGAAGATTTTTCAACTTAT | | SSLQPEDFSTYYCQQ | | | |
| | | TACTGTCAACAGTATTATAGTAACCTCGGGAGGACGTTCGGCC | | YYSNLGRTFGQGTKV | | | |
| | | AAGGGACCAAGGTGGATATCAAA | | DIK | | | |
| ADI-81573 | 2949 | TCGTATGAGCTGACTCAGCCACCCTCAGCGTCTGGGACCCCCG | 4868 | SYELTQPPSASGTPG | 5221 | 3 | 3 |
| | | GGCAGAGGGTCACCATCTCTTGTTCTGGAAGCAGCTCCAACAT | | QRVTISCSGSSSNIG | | | |
| | | CGGAAGTAATACTTTAAACTGGTACCAGCAGCTCCCAGGAACG | | SNTLNWYQQLPGTAP | | | |
| | | GCCCCCAAACTCCTCATCTATAATAATAATCAGCGGCCCTCAG | | KLLIYNNNQRPSGVP | | | |
| | | GGGTCCCTGACCGATTCTCTGGCTCCAAGTCTGGCACCTCAGC | | DRFSGSKSGTSASLA | | | |
| | | CTCCCTGGCCATCAGGGGGCTCCAGTCTGAGGATGAGGCTGAT | | IRGLQSEDEADYYCA | | | |
| | | TATTACTGTGCAGCATGGGATGACAGCCTGAATGGTTATGTCT | | AWDDSLNGYVFGTGT | | | |
| | | TCGGAACTGGGACCAAGCTCACCGTCCTA | | KLTVL | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81614 | IGKV1D-39 | DIQMTQ | 1878 | RASQRI | 2135 | WYQQKP | 2257 | AASTLH | 4055 | GVPSRF | 2534 | QQSYST | 2715 | FGPGTR |
| | | SPSSLS | | SSYLN | | GKAPKL | | S | | SGSGSG | | PIT | | LEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81635 | IGLV3-21 | SYELTQ | 1919 | GGNDIG | 3876 | WYQQKP | 2283 | DDSDRP | 2399 | GIPERF | 4303 | QLWDST | 4523 | FGGGTK |
| | | PPSVSV | | SKNVH | | GQAPVL | | S | | SGSNSG | | GDHPEV | | LTVL |
| | | APGQTA | | | | VVY | | | | NTATLT | | IL | | |
| | | RITC | | | | | | | | ISRVEG | | | | |
| | | | | | | | | | | GDEADY | | | | |
| | | | | | | | | | | YC | | | | |
| ADI-81652 | IGKV2D-28 | EIVMTQ | 1923 | RSSQSL | 3877 | WYLQKP | 2260 | LASNRA | 4143 | GVPDRF | 4304 | MQALQT | 2765 | FGQGTR |
| | | SPLSLP | | LHSNGH | | GQSPQL | | S | | SGSGSG | | PIT | | LEIK |
| | | VTPGEP | | NYLD | | LIY | | | | TDFTLR | | | | |
| | | ASISC | | | | | | | | ISRVEA | | | | |
| | | | | | | | | | | EDVGVY | | | | |
| | | | | | | | | | | YC | | | | |
| ADI-81906 | IGKV3D-15 | EMTLTQ | 3675 | RASQSV | 3878 | WYQQKP | 2258 | GASTRA | 2383 | GIPARF | 4305 | QQYNIQ | 4524 | FGQGTK |
| | | SPATLS | | SNNLA | | GQAPRL | | T | | SGSGSG | | WT | | VEIK |
| | | VSPGGR | | | | LIY | | | | TEFTLT | | | | |
| | | ATLSC | | | | | | | | ISSLQA | | | | |
| | | | | | | | | | | EDFAVY | | | | |
| | | | | | | | | | | YC | | | | |
| ADI-81578 | IGLV1-40 | QSVLTQ | 1948 | TGSNSN | 2143 | WYQQLP | 2314 | GNTNRP | 2484 | GVPDRF | 2666 | QSYDSS | 4525 | FGGGTK |
| | | PPSVSG | | IGAGYD | | GTAPKL | | S | | SGSRSG | | LSTVF | | LTVL |
| | | APGQRV | | VH | | LIY | | | | TSASLA | | | | |
| | | TISC | | | | | | | | ITGLQA | | | | |
| | | | | | | | | | | EDEADY | | | | |
| | | | | | | | | | | YC | | | | |
| ADI-81856 | IGKV3D-15 | EMTLTQ | 3676 | RASQSV | 2227 | WYQQKP | 4015 | GASTRA | 2383 | GIPARF | 2569 | QQYHSW | 4526 | FGPGTK |
| | | SPGTLS | | SSNLA | | GQAPRL | | T | | SGSGSG | | PLFT | | VEIK |
| | | VSPGKR | | | | FIY | | | | TEFTLT | | | | |
| | | ATLSC | | | | | | | | ISSLQS | | | | |
| | | | | | | | | | | EDFAVY | | | | |
| | | | | | | | | | | YC | | | | |
| ADI-81896 | IGKV3D-15 | EIVMTQ | 3677 | RASQSV | 3879 | WYQQKP | 2258 | GASTRA | 2383 | GIPARF | 2529 | HQYTHW | 4527 | FGPGTK |
| | | SPATLS | | RSNLA | | GQAPRL | | T | | SGSGSG | | PLT | | VEIK |
| | | VSPGER | | | | LIY | | | | TEFTLT | | | | |
| | | VTLSC | | | | | | | | ISSLQS | | | | |
| | | | | | | | | | | EDFAVY | | | | |
| | | | | | | | | | | FC | | | | |
| ADI-81567 | IGLV1-40 | QSVMTQ | 3678 | TGSNSN | 2143 | WYQQLP | 3992 | VNSNRP | 4113 | GVPDRF | 2599 | QSYDSA | 4528 | FGGGTK |
| | | PPSVSG | | IGAGYD | | GTAPKL | | S | | SGSKSG | | LSGSV | | LTVL |
| | | APGQRV | | VH | | LIS | | | | TSASLA | | | | |
| | | TISC | | | | | | | | ITGLQA | | | | |
| | | | | | | | | | | EDEADY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81614 | 4593 | GACATCCAGATGACCCAGTCTCCATCCTCCCTGTCTGCATCTG | 4869 | DIQMTQSPSSLSASV | 5222 | 3 | 3 |
| | | TAGGAGACAGAGTCACCATCACTTGCCGGGCAAGTCAGCGCAT | | GDRVTITCRASQRIS | | | |
| | | TAGCAGCTATTTAAATTGGTATCAGCAGAAACCAGGGAAAGCC | | SYLNWYQQKPGKAPK | | | |
| | | CCTAAGCTCCTGATCTATGCTGCATCCACTTTGCACAGTGGGG | | LLIYAASTLHSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAACCTGAAGATTTTGCAACTTAC | | SSLQPEDFATYYCQQ | | | |
| | | TACTGTCAACAGAGTTACAGTACCCCGATCACCTTCGGCCCAG | | SYSTPITFGPGTRLE | | | |
| | | GGACACGACTGGAGATTAAA | | IK | | | |
| ADI-81635 | 2939 | TCGTATGAGCTGACGCAGCCACCCTCGGTGTCAGTGGCCCCAG | 4870 | SYELTQPPSVSVAPG | 5223 | 3 | 3 |
| | | GGCAGACGGCCAGGATTACCTGTGGGGGAAACGACATTGGAAG | | QTARITCGGNDIGSK | | | |
| | | TAAAAATGTGCACTGGTACCAACAGAAGCCAGGCCAGGCCCCT | | NVHWYQQKPGQAPVL | | | |
| | | GTGTTGGTCGTCTATGATGATAGCGACCGGCCCTCAGGGATCC | | VVYDDSDRPSGIPER | | | |
| | | CTGAGCGATTCTCTGGCTCCAACTCTGGGAACACGGCCACCCT | | FSGSNSGNTATLTIS | | | |
| | | GACCATCAGCAGGGTCGAAGGCGGGGATGAGGCCGACTATTAC | | RVEGGDEADYYCQLW | | | |
| | | TGTCAGCTGTGGGATAGTACTGGTGATCATCCGGAGGTGTTAT | | DSTGDHPEVLFGGGT | | | |
| | | TCGGCGGAGGGACCAAGCTCACCGTCCTA | | KLTVL | | | |
| ADI-81652 | 2940 | GAAATTGTGATGACTCAGTCTCCACTCTCCCTGCCCGTCACCC | 4871 | EIVMTQSPLSLPVTP | 5224 | 3 | 3 |
| | | CTGGAGAGCCGGCCTCCATCTCCTGCAGGTCTAGTCAGAGCCT | | GEPASISCRSSQSLL | | | |
| | | CCTGCATAGTAATGGACACAACTATTTGGATTGGTACCTGCAG | | HSNGHNYLDWYLQKP | | | |
| | | AAGCCAGGGCAGTCTCCACAACTCCTGATCTATTTGGCTTCTA | | GQSPQLLIYLASNRA | | | |
| | | ATCGGGCCTCCGGGGTCCCTGACAGGTTCAGTGGCAGTGGATC | | SGVPDRFSGSGSGTD | | | |
| | | AGGCACAGATTTTACACTGAGAATCAGCAGAGTGGAGGCTGAG | | FTLRISRVEAEDVGV | | | |
| | | GATGTTGGGGTTTATTACTGCATGCAAGCTCTACAAACTCCGA | | YYCMQALQTPITFGQ | | | |
| | | TCACCTTCGGCCAAGGGACACGACTGGAGATTAAA | | GTRLEIK | | | |
| ADI-81906 | 2937 | GAAATGACACTCACGCAGTCTCCAGCCACCCTGTCTGTGTCTC | 4872 | EMTLTQSPATLSVSP | 5225 | 3 | 3 |
| | | CAGGGGGAAGAGCCACCCTCTCCTGCAGGGCCAGTCAGAGTGT | | GGRATLSCRASQSVS | | | |
| | | TAGCAACAACTTAGCCTGGTACCAGCAGAAACCTGGCCAGGCT | | NNLAWYQQKPGQAPR | | | |
| | | CCCAGGCTCCTCATCTATGGTGCATCCACCAGGGCCACTGGTA | | LLIYGASTRATGIPA | | | |
| | | TCCCAGCCAGGTTCAGTGGCAGCGGGTCTGGGACAGAGTTCAC | | RFSGSGSGTEFTLTI | | | |
| | | TCTCACCATCAGCAGCCTGCAGGCTGAAGACTTTGCAGTTTAT | | SSLQAEDFAVYYCQQ | | | |
| | | TACTGTCAGCAGTATAATATCCAGTGGACGTTCGGCCAAGGGA | | YNIQWTFGQGTKVEI | | | |
| | | CCAAGGTGGAAATCAAA | | K | | | |
| ADI-81578 | 2939 | CAGTCTGTGCTGACGCAGCCGCCCTCAGTGTCTGGGGCCCCAG | 4873 | QSVLTQPPSVSGAPG | 5226 | 3 | 3 |
| | | GGCAGAGGGTCACCATCTCCTGCACTGGGAGCAACTCCAACAT | | QRVTISCTGSNSNIG | | | |
| | | CGGGGCAGGTTATGATGTACACTGGTACCAGCAGCTTCCAGGA | | AGYDVHWYQQLPGTA | | | |
| | | ACAGCCCCCAAACTCCTCATCTATGGTAACACCAATCGTCCCT | | PKLLIYGNTNRPSGV | | | |
| | | CAGGGGTCCCTGACCGATTCTCTGGCTCCAGGTCTGGCACCTC | | PDRFSGSRSGTSASL | | | |
| | | AGCCTCCCTGGCCATCACTGGGCTCCAGGCTGAGGATGAGGCT | | AITGLQAEDEADYYC | | | |
| | | GATTATTACTGCCAGTCTTATGACAGCAGCCTGAGTACTGTGT | | QSYDSSLSTVFFGGG | | | |
| | | TCTTCGGCGGAGGGACCAAGCTCACCGTCCTA | | TKLTVL | | | |
| ADI-81856 | 2942 | GAAATGACGCTGACGCAGTCTCCAGGCACCCTGTCTGTGTCTC | 4874 | EMTLTQSPGTLSVSP | 5227 | 3 | 3 |
| | | CAGGAAAACGAGCCACCCTCTCCTGCAGGGCCAGTCAGAGTGT | | GKRATLSCRASQSVS | | | |
| | | TAGCAGCAACTTAGCCTGGTACCAGCAGAAACCTGGCCAGGCT | | SNLAWYQQKPGQAPR | | | |
| | | CCCAGGCTCTTCATTTATGGTGCATCCACCAGGGCCACTGGTA | | LFIYGASTRATGIPA | | | |
| | | TCCCAGCCAGGTTCAGTGGCAGTGGGTCTGGGACAGAGTTCAC | | RFSGSGSGTEFTLTI | | | |
| | | TCTCACCATCAGCAGCCTGCAGTCTGAAGATTTTGCAGTTTAT | | SSLQSEDFAVYYCQQ | | | |
| | | TACTGTCAGCAGTATCATAGCTGGCCTTTATTCACTTTCGGCC | | YHSWPLFTFGPGTKV | | | |
| | | CTGGGACCAAGGTGGAAATCAAA | | EIK | | | |
| ADI-81896 | 2942 | GAAATCGTAATGACGCAGTCTCCAGCCACCCTGTCTGTGTCTC | 4875 | EIVMTQSPATLSVSP | 5228 | 3 | 3 |
| | | CAGGGGAAAGAGTCACCCTCTCCTGCAGGGCCAGTCAGAGTGT | | GERVTLSCRASQSVR | | | |
| | | TAGAAGCAACTTAGCCTGGTACCAACAAAAACCTGGCCAGGCT | | SNLAWYQQKPGQAPR | | | |
| | | CCCAGGCTCCTCATTTACGGTGCATCCACCAGGGCCACTGGTA | | LLIYGASTRATGIPA | | | |
| | | TCCCAGCCAGGTTCAGTGGCAGTGGGTCTGGGACAGAGTTCAC | | RFSGSGSGTEFTLTI | | | |
| | | TCTCACCATCAGCAGCCTGCAGTCTGAAGATTTTGCAGTTTAT | | SSLQSEDFAVYFCHQ | | | |
| | | TTCTGTCACCAGTATACTCACTGGCCTCTCACTTTCGGCCCTG | | YTHWPLTFGPGTKVE | | | |
| | | GGACCAAAGTGGAGATCAAA | | | | | |
| ADI-81567 | 2939 | CAGTCTGTGATGACTCAGCCGCCCTCAGTGTCTGGGGCCCCAG | 4876 | QSVMTQPPSVSGAPG | 5229 | 3 | 3 |
| | | GGCAGAGGGTCACCATCTCCTGCACTGGGAGCAACTCCAACAT | | QRVTISCTGSNSNIG | | | |
| | | CGGGGCAGGCTATGATGTACACTGGTACCAGCAGCTTCCAGGA | | AGYDVHWYQQLPGTA | | | |
| | | ACAGCCCCCAAACTCCTCATCTCTGTTAACAGCAATCGGCCCT | | PKLLISVNSNRPSGV | | | |
| | | CAGGGGTCCCTGACCGATTTTCTGGCTCCAAGTCTGGCACTTC | | PDRFSGSKSGTSASL | | | |
| | | AGCCTCCCTGGCCATCACTGGGCTCCAGGCTGAGGATGAGGCT | | AITGLQAEDEADYYC | | | |
| | | GATTATTACTGCCAGTCCTATGACAGCGCCCTGAGTGGTTCGG | | QSYDSALSGSVFGGG | | | |
| | | TATTCGGCGGAGGGACCAAGCTGACCGTCCTA | | TKLKLTVL | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81721 | IGKV1D-39 | EITMTQ | 3637 | RASQSI | 2066 | WYQQKP | 2257 | AASSLQ | 2391 | MVPSRF | 4306 | QHSYST | 4529 | FGQGTK |
| | | SPSSLS | | STYLN | | GKAPKL | | S | | SGSGSG | | PPWT | | VEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81789 | IGKV1D-39 | DIRMTQ | 1937 | RASQTI | 3880 | WYQQKP | 2257 | GASSLQ | 2403 | GVPSRF | 4307 | QQSYTM | 4530 | FGPGTK |
| | | SPSSLS | | SSYLN | | GKAPKL | | S | | SGSGSG | | PPIT | | VDIK |
| | | ASVGDR | | | | LIY | | | | TDFTLT | | | | |
| | | VTITC | | | | | | | | ISSLQA | | | | |
| | | | | | | | | | | EDFATY | | | | |
| | | | | | | | | | | YC | | | | |
| ADI-81559 | IGKV1D-39 | DIQMTQ | 1878 | RASQSI | 2117 | WYQQKP | 2263 | DASSLQ | 2389 | GVPSRF | 2534 | QQSYNT | 2702 | FGRGTK |
| | | SPSSLS | | RSYLN | | GKAPNL | | S | | SGSGSG | | PPWT | | VEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81642 | IGLV3-21 | QPVLTQ | 1982 | GGDNIG | 2180 | WYQQKP | 2283 | DDSDRP | 2399 | GIPERF | 2643 | QVWDST | 2836 | FGTGTK |
| | | PPSVSV | | SKGVH | | GQAPVL | | S | | SGSNSG | | SDHPAY | | VTVL |
| | | APGQTA | | | | VVY | | | | NTATLT | | V | | |
| | | RITC | | | | | | | | ISRVEA | | | | |
| | | | | | | | | | | GDEADY | | | | |
| | | | | | | | | | | FC | | | | |
| ADI-81799 | IGKV3-11 | EMTLTQ | 3534 | RASQSL | 3881 | WYQQKP | 2258 | DASNRA | 2448 | GIPARF | 4308 | QQRSSW | 4531 | FGQGTK |
| | | SPATLS | | SGYLA | | GQAPRL | | T | | SGSGSG | | PRLYT | | VEIK |
| | | LSPGER | | | | LIY | | | | TDFTLT | | | | |
| | | ATLSC | | | | | | | | ISSLEP | | | | |
| | | | | | | | | | | EDFALY | | | | |
| | | | | | | | | | | YC | | | | |
| ADI-81913 | IGKV1-NL1 | DIQMTQ | 1878 | RASQGI | 3882 | WYQQKP | 2303 | VASRLE | 2411 | GVPSRF | 2531 | QQYYNT | 2732 | FGQGTK |
| | | SPSSLS | | SYSLA | | GKAPKL | | S | | SGSGSG | | PVT | | VEIK |
| | | ASVGDR | | | | LVY | | | | TDYTLT | | | | |
| | | VTITC | | | | | | | | ISSLQP | | | | |
| | | | | | | | | | | EDFATY | | | | |
| | | | | | | | | | | YC | | | | |
| ADI-81546 | IGKV1-5 | EIQLTQ | 3679 | RASQSI | 2190 | WYQQKP | 2257 | EASSLD | 4144 | GVPSRF | 2532 | QQYNSY | 4532 | FGGGTK |
| | | SPSTLS | | SSWLA | | GKAPKL | | T | | SGSGSG | | LT | | VDIK |
| | | ASVGDR | | | | LIY | | | | TEFTLT | | | | |
| | | VTITC | | | | | | | | ISSLQP | | | | |
| | | | | | | | | | | DDFATY | | | | |
| | | | | | | | | | | YC | | | | |
| ADI-81563 | IGLV1-40 | QPGLTQ | 3680 | TGSNSN | 2143 | WYQQLP | 2314 | DNINRP | 4110 | GVPDRF | 2599 | QSYDSS | 2864 | FGTATK |
| | | PPSVSG | | IGAGYD | | GTAPKL | | S | | SGSKSG | | LIGSV | | VTVL |
| | | APGQRV | | VH | | LIY | | | | TSASLA | | | | |
| | | TISC | | | | | | | | ITGLQA | | | | |
| | | | | | | | | | | EDEADY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81721 | 2937 | GAAATCACGATGACCCAGTCTCCATCCTCCCTGTCTGCATCTG | 4877 | EITMTQSPSSLSASV | 5230 | 3 | 2 |
| | | TAGGAGACAGAGTCACCATCACTTGCCGGGCAAGTCAGAGCAT | | GDRVTITCRASQSIS | | | |
| | | TAGCACCTATTTAAATTGGTATCAGCAGAAACCAGGGAAAGCC | | TYLNWYQQKPGKAPK | | | |
| | | CCTAAGCTCCTGATCTATGCTGCATCCAGTTTGCAAAGTATGG | | LLIYAASSLQSMVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAACCTGAAGATTTTGCTACTTAC | | SSLQPEDFATYYCQH | | | |
| | | TACTGTCAACACAGTTACAGTACGCCTCCGTGGACCTTCGGCC | | SYSTPPWTFGQGTKV | | | |
| | | AAGGGACCAAGGTGGAAATCAAA | | EIK | | | |
| ADI-81789 | 2950 | GACATCCGGATGACCCAGTCTCCATCTTCCCTGTCTGCATCTG | 4878 | DIRMTQSPSSLSASV | 5231 | 3 | 3 |
| | | TAGGAGACAGAGTCACCATCACTTGCCGGGCAAGTCAGACCAT | | GDRVTITCRASQTIS | | | |
| | | TAGCAGCTATTTAAATTGGTATCAGCAGAAACCAGGGAAAGCC | | SYLNWYQQKPGKAPK | | | |
| | | CCTAAGCTCCTGATCTATGGTGCATCCAGTTTGCAAAGTGGGG | | LLIYGASSLQSGVPS | | | |
| | | TCCCATCAAGGTTCAGTGGCAGTGGCTCTGGGACAGATTTCAC | | RFSGSGSGTDFTLTI | | | |
| | | TCTCACCATCAGCAGTCTGCAAGCTGAAGATTTTGCAACTTAC | | SSLQAEDFATYYCQQ | | | |
| | | TACTGTCAACAGAGTTACACTATGCCTCCGATCACCTTCGGCC | | SYTMPPITFGPGTKV | | | |
| | | CTGGGACCAAAGTGGATATCAAA | | DIK | | | |
| ADI-81559 | 2941 | GACATCCAGATGACCCAGTCTCCATCCTCCCTGTCTGCATCTG | 4879 | DIQMTQSPSSLSASV | 5232 | 3 | 3 |
| | | TAGGAGACAGAGTCACCATCACTTGCCGGGCAAGTCAGAGCAT | | GDRVTITCRASQSIR | | | |
| | | TAGGAGCTATTTAAATTGGTATCAGCAGAAACCAGGGAAAGCC | | SYLNWYQQKPGKAPN | | | |
| | | CCTAACCTCCTGATCTATGATGCATCCAGTTTGCAAAGTGGAG | | LLIYDASSLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAACCTGAAGATTTTGCAACTTAC | | SSLQPEDFATYYCQQ | | | |
| | | TACTGTCAACAGAGTTACAATACCCCCCCGTGGACGTTCGGCC | | SYNTPPWTFGRGTKV | | | |
| | | GAGGGACCAAGGTGGAAATCAAA | | EIK | | | |
| ADI-81642 | 2945 | CAGCCTGTGCTGACGCAGCCACCCTCGGTGTCAGTGGCCCCGG | 4880 | QPVLTQPPSVSVAPG | 5233 | 3 | 3 |
| | | GACAGACGGCCAGGATTACCTGTGGGGGAGACAACATTGGGAG | | QTARITCGGDNIGSK | | | |
| | | CAAAGGTGTTCACTGGTACCAGCAGAAGCCAGGCCAGGCCCCT | | GVHWYQQKPGQAPVL | | | |
| | | GTGTTGGTCGTCTATGATGATAGCGACCGGCCCTCAGGGATCC | | VVYDDSDRPSGIPER | | | |
| | | CTGAGCGATTCTCTGGCTCCAACTCGGGGAACACGGCCACCCT | | FSGSNSGNTATLTIS | | | |
| | | GACCATCAGCAGGGTCGAAGCCGGGGATGAGGCCGACTACTTC | | RVEAGDEADYFCQVW | | | |
| | | TGTCAGGTGTGGGATAGTACGAGTGATCATCCGGCTTATGTCT | | DSTSDHPAYVFGTGT | | | |
| | | TCGGAACTGGGACCAAGGTCACCGTCCTA | | KVTVL | | | |
| ADI-81799 | 2937 | GAAATGACACTCACCCAGTCTCCAGCCACCCTGTCTTTGTCTC | 4881 | EMTLTQSPATLSLSP | 5234 | 3 | 3 |
| | | CAGGGGAAAGAGCCACCCTCTCCTGCAGGGCCAGTCAGAGTCT | | GERATLSCRASQSLS | | | |
| | | TAGCGGCTACTTAGCCTGGTACCAACAGAAACCTGGCCAGGCT | | GYLAWYQQKPGQAPR | | | |
| | | CCCAGGCTCCTCATCTATGATGCATCCAACAGGGCCACTGGCA | | LLIYDASNRATGIPA | | | |
| | | TCCCAGCCAGGTTCAGTGGCAGTGGGTCTGGGACAGACTTCAC | | RFSGSGSGTDFTLTI | | | |
| | | TCTCACCATCAGCAGCCTAGAGCCTGAAGATTTTGCACTTTAT | | SSLEPEDFALYYCQQ | | | |
| | | TACTGTCAACAGCGTAGCAGCTGGCCTCGGTTGTACACTTTTG | | RSSWPRLYTFGQGTK | | | |
| | | GCCAGGGGACCAAGGTGGAGATCAAA | | VEIK | | | |
| ADI-81913 | 2937 | GACATCCAGATGACCCAGTCTCCATCCTCCCTGTCTGCATCTG | 4882 | DIQMTQSPSSLSASV | 5235 | 3 | 3 |
| | | TAGGAGACAGAGTCACCATCACTTGCCGGGCGAGTCAGGGCAT | | GDRVTITCRASQGIS | | | |
| | | TAGCTATTCTTTAGCCTGGTATCAGCAGAAACCAGGGAAAGCC | | YSLAWYQQKPGKAPK | | | |
| | | CCTAAGCTCCTGGTCTATGTTGCATCCAGATTGGAAAGTGGCG | | LLVYVASRLESGVPS | | | |
| | | TCCCATCCAGGTTCAGTGGCAGTGGATCTGGGACAGATTACAC | | RFSGSGSGTDYTLTI | | | |
| | | TCTCACCATCAGCAGCCTGCAGCCTGAAGATTTTGCAACTTAT | | SSLQPEDFATYYCQQ | | | |
| | | TACTGTCAACAGTATTATAATACCCCCGTGACGTTCGGCCAAG | | YYNTPVTFGQGTKVE | | | |
| | | GGACCAAGGTGGAAATCAAA | | IK | | | |
| ADI-81546 | 2938 | GAAATCCAATTGACCCAGTCTCCTTCCACCCTGTCTGCATCTG | 4883 | EIQLTQSPSTLSASV | 5236 | 3 | 3 |
| | | TAGGAGACAGAGTCACCATCACTTGCCGGGCCAGTCAGAGTAT | | GDRVTITCRASQSIS | | | |
| | | TAGTAGCTGGTTGGCCTGGTATCAGCAGAAACCAGGGAAAGCC | | SWLAWYQQKPGKAPK | | | |
| | | CCTAAGCTCCTGATCTATGAGGCGTCTAGTTTAGATACTGGGG | | LLIYEASSLDTGVPS | | | |
| | | TCCCATCAAGGTTCAGCGGCAGTGGATCTGGGACAGAATTCAC | | RFSGSGSGTEFTLTI | | | |
| | | TCTCACCATCAGCAGCCTGCAGCCTGATGATTTTGCAACTTAT | | SSLQPDDFATYYCQQ | | | |
| | | TACTGCCAACAGTATAATAGTTATCTCACTTTCGGCGGAGGGA | | YNSYLTFGGGTKVDI | | | |
| | | CCAAGGTGGATATCAAA | | K | | | |
| ADI-81563 | 4601 | CAGCCAGGGCTGACTCAGCCACCCTCAGTGTCTGGGGCCCCAG | 4884 | QPGLTQPPSVSGAPG | 5237 | 3 | 3 |
| | | GGCAGAGGGTCACCATCTCCTGCACTGGGAGCAACTCCAACAT | | QRVTISCTGSNSNIG | | | |
| | | CGGGGCAGGTTATGATGTACACTGGTACCAGCAGCTTCCAGGA | | AGYDVHWYQQLPGTA | | | |
| | | ACAGCCCCCAAACTCCTCATCTATGATAACATCAATCGGCCCT | | PKLLIYDNINRPSGV | | | |
| | | CAGGGGTCCCTGACCGATTCTCTGGCTCCAAGTCTGGCACCTC | | PDRFSGSKSGTSASL | | | |
| | | AGCCTCCCTGGCCATCACTGGGCTCCAGGCTGAGGATGAGGCT | | AITGLQAEDEADYYC | | | |
| | | GATTATTACTGCCAGTCCTATGACAGCAGCCTCATTGGTTCTG | | QSYDSSLIGSVFGTA | | | |
| | | TCTTCGGAACTGCGACCAAGGTCACCGTCCTA | | TKVTVL | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81596 | IGKV3-20 | EIPMTQ | 3681 | RASQSV- | 2163 | WYQQKP | 2258 | GASSRA | 2464 | GIPDRF | 2616 | QQYGSS | 2780 | FGQGTK |
| | | SPGTLS | | SSYLA | | GQAPRL | | T | | SGSGSG | | PRT | | VDIK |
| | | LSPGER | | | | LIY | | | | TDFTLT | | | | |
| | | ATLSC | | | | | | | | ISRLEP | | | | |
| | | | | | | | | | | EDFAVY | | | | |
| | | | | | | | | | | YC | | | | |
| ADI-81900 | IGKV1-16 | EIVMTQ | 1891 | RASQGI | 3766 | WFQQKP | 2346 | AASSLQ | 4145 | GVPSKF | 4309 | QQYHSY | 4533 | FGQGTK |
| | | SPSSLS | | NNFLA | | GKAPKS | | N | | SGSGSG | | PQT | | VDIK |
| | | ASVGDR | | | | LIY | | | | TDFTLT | | | | |
| | | VTITQ | | | | | | | | ISSLQP | | | | |
| | | | | | | | | | | EDFATY | | | | |
| | | | | | | | | | | YC | | | | |
| ADI-81581 | IGLV5-37 | QPVLTQ | 3682 | TLPSDI | 3883 | WYQQKP | 4016 | YYSDSD | 4146 | GVPSRF | 4310 | MIWPSN | 4534 | FGGGTK |
| | | PPSSSA | | NVGSYN | | GRPPRY | | KGQGS | | SGSKDA | | LLGV | | LTVL |
| | | SPGESA | | VY | | LLY | | | | SANTGI | | | | |
| | | RLTC | | | | | | | | LLISGL | | | | |
| | | | | | | | | | | QSEDEA | | | | |
| | | | | | | | | | | DYYC | | | | |
| ADI-81615 | IGLV1-40 | QSVLTQ | 1948 | TGSNSN | 2143 | WYQQLP | 2314 | GNNNRP | 2492 | GVPDRF | 2695 | QSYDSS | 2840 | FGGGTK |
| | | PPSVSG | | IGAGYD | | GTAPKL | | S | | SGSKSV | | LSDSV | | LTVL |
| | | APGQRV | | VH | | LIY | | | | TSASLA | | | | |
| | | TISC | | | | | | | | ITGLQA | | | | |
| | | | | | | | | | | EDEADY | | | | |
| | | | | | | | | | | YC | | | | |
| ADI-81904 | IGKV1-9 | DIRMTQ | 3683 | RASQDI | 3785 | WYQQKR | 4017 | AASTLQ | 2392 | GVPSRF | 2592 | QQLNSY | 4535 | FGQGTR |
| | | SPSFLS | | SSYLA | | GKAPKV | | S | | SGSGSG | | PLT | | LEIK |
| | | ASVGDR | | | | LIY | | | | TEFTLT | | | | |
| | | VTITC | | | | | | | | ISSLQP | | | | |
| | | | | | | | | | | EDFATY | | | | |
| | | | | | | | | | | YC | | | | |
| ADI-81818 | IGKV1-12 | DIQMTQ | 1885 | RASQGI | 3884 | WYQQKP | 2257 | AATTLQ | 4122 | GVPSRF | 2534 | QQANSF | 4536 | FGQGTK |
| | | SPSSVS | | SGWLA | | GKAPKL | | S | | SGSGSG | | PRT | | VEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81540 | IGLV3-21 | SYVLTQ | 3684 | GGNNIG | 2151 | WYQQKP | 2304 | YDSDRP | 2498 | GIPERF | 2545 | QVWDSN | 2881 | FGGGTK |
| | | PPSVSV | | SKHVH | | GQAPVL | | S | | SGSNSG | | SDHRV | | VTVL |
| | | APGKTA | | | | VIY | | | | NTATLT | | | | |
| | | KITC | | | | | | | | ISRVEA | | | | |
| | | | | | | | | | | GDEADY | | | | |
| | | | | | | | | | | YC | | | | |
| ADI-81880 | IGKV3-20 | EMTMTQ | 1990 | RASQSV | 3885 | WYQHKP | 2328 | GASSRA | 2464 | GIPDRF | 2616 | QQYDSS | 4537 | FGGGTK |
| | | SPGTLS | | SSSFLA | | GQAPRL | | T | | SGSGSG | | PLT | | VEIK |
| | | LSPGER | | | | LIY | | | | TDFTLT | | | | |
| | | ATLSC | | | | | | | | ISRLEP | | | | |
| | | | | | | | | | | EDFAVY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81596 | 2944 | GAAATCCCAATGACACAGTCTCCAGGCACCCTGTCTTTGTCTC | 4885 | EIPMTQSPGTLSLSP | 5238 | 3 | 1 |
| | | CAGGGGAAAGAGCCACCCTCTCCTGCAGGGCCAGTCAGAGTGT | | GERATLSCRASQSVS | | | |
| | | TAGCAGCTACTTAGCCTGGTACCAACAGAAACCTGGCCAGGCT | | SYLAWYQQKPGQAPR | | | |
| | | CCCAGGCTCCTCATCTATGGTGCATCCAGCAGGGCCACTGGCA | | LLIYGASSRATGIPD | | | |
| | | TCCCAGACAGGTTCAGTGGCAGTGGGTCTGGGACAGACTTCAC | | RFSGSGSGTDFTLTI | | | |
| | | TCTCACCATCAGCAGACTGGAGCCTGAAGATTTTGCAGTGTAT | | SRLEPEDFAVYYCQQ | | | |
| | | TACTGTCAGCAGTATGGTAGCTCACCGAGGACGTTCGGCCAAG | | YGSSPRTFGQGTKVD | | | |
| | | GGACCAAAGTGGATATCAAA | | IK | | | |
| ADI-81900 | 2944 | GAAATCGTGATGACCCAGTCTCCATCCTCACTGTCTGCATCTG | 4886 | EIVMTQSPSSLSASV | 5239 | 3 | 3 |
| | | TAGGAGACAGAGTCACCATCACTTGTCGGGCGAGTCAGGGCAT | | GDRVTITCRASQGIN | | | |
| | | TAACAATTTTTTAGCCTGGTTTCAGCAGAAACCAGGGAAAGCC | | NFLAWFQQKPGKAPK | | | |
| | | CCTAAGTCCCTGATCTATGCTGCATCCAGTTTGCAAAATGGGG | | SLIYAASSLQNGVPS | | | |
| | | TCCCATCAAAGTTCAGCGGCAGTGGATCTGGGACAGATTTCAC | | KFSGSGSGTDFTLTI | | | |
| | | TCTCACCATCAGCAGCCTGCAGCCTGAAGATTTTGCAACTTAT | | SSLQPEDFATYYCQQ | | | |
| | | TACTGCCAACAGTATCATAGTTACCCTCAGACGTTCGGCCAAG | | YHSYPQTFGQGTKVD | | | |
| | | GGACCAAAGTGGATATCAAA | | IK | | | |
| ADI-81581 | 2939 | CAGCCTGTGCTGACTCAGCCACCTTCCTCCTCCGCATCTCCTG | 4887 | QPVLTQPPSSSASPG | 5240 | 3 | 2 |
| | | GAGAATCCGCCAGACTCACCTGCACCTTGCCCAGTGACATCAA | | ESARLTCTLPSDINV | | | |
| | | TGTTGGTAGTTACAACGTGTACTGGTACCAGCAGAAGCCAGGG | | GSYNVYWYQQKPGRP | | | |
| | | CGCCCTCCCAGGTATCTCCTGTACTACTACTCAGACTCAGATA | | PRYLLYYYSDSDKGQ | | | |
| | | AGGGCCAGGGCTCTGGAGTCCCCAGCCGCTTCTCTGGATCCAA | | GSGVPSRFSGSKDAS | | | |
| | | AGATGCTTCAGCCAATACAGGGATTTTACTCATCTCCGGGCTC | | ANTGILLISGLQSED | | | |
| | | CAGTCTGAGGATGAGGCTGACTATTACTGTATGATTTGGCCAA | | EADYYCMIWPSNLLG | | | |
| | | GCAATCTTCTAGGGGTGTTCGGCGGAGGGACCAAGCTCACCGT | | VFGGGTKLTVL | | | |
| | | CCTA | | | | | |
| ADI-81615 | 2939 | CAGTCTGTGTTGACGCAGCCGCCCTCAGTGTCTGGGGCCCCAG | 4888 | QSVLTQPPSVSGAPG | 5241 | 3 | 3 |
| | | GGCAGAGGGTCACCATCTCCTGCACTGGGAGCAACTCCAACAT | | QRVTISCTGSNSNIG | | | |
| | | CGGGGCAGGTTATGATGTACACTGGTACCAGCAACTTCCAGGA | | AGYDVHWYQQLPGTA | | | |
| | | ACAGCCCCCAAACTCCTCATCTATGGTAACAACAATCGGCCCT | | PKLLIYGNNNRPSGV | | | |
| | | CAGGGGTCCCTGACCGATTCTCTGGCTCCAAGTCTGTCACCTC | | PDRFSGSKSVTSASL | | | |
| | | AGCCTCCCTGGCCATCACTGGGCTCCAGGCTGAGGATGAGGCT | | AITGLQAEDEADYYC | | | |
| | | GATTATTACTGCCAGTCCTATGACAGCAGCCTGAGTGATTCGG | | QSYDSSLSDSVFGGG | | | |
| | | TGTTCGGCGGAGGGACCAAGCTGACCGTCCTA | | TKLTVL | | | |
| ADI-81904 | 2940 | GACATCCGGATGACCCAGTCTCCATCCTTCCTGTCTGCATCTG | 4889 | DIRMTQSPSFLSASV | 5242 | 3 | 3 |
| | | TAGGAGACAGAGTCACCATCACTTGCCGGGCCAGTCAGGACAT | | GDRVTITCRASQDIS | | | |
| | | TAGCAGTTATTTAGCCTGGTATCAGCAAAAACGAGGGAAAGCC | | SYLAWYQQKRGKAPK | | | |
| | | CCTAAGGTCCTGATCTATGCTGCATCCACTTTGCAAAGTGGGG | | VLIYAASTLQSGVPS | | | |
| | | TCCCATCAAGGTTCAGCGGCAGTGGATCTGGGACAGAATTCAC | | RFSGSGSGTEFTLTI | | | |
| | | TCTCACAATCAGCAGCCTGCAGCCTGAAGATTTTGCAACTTAT | | SSLQPEDFATYYCQQ | | | |
| | | TACTGTCAACAACTTAATAGTTACCCTCTCACCTTCGGCCAAG | | LNSYPLTFGQGTRLE | | | |
| | | GGACACGACTGGAGATTAAA | | IK | | | |
| ADI-81818 | 2937 | GACATCCAGATGACCCAGTCTCCATCTTCCGTGTCTGCATCTG | 4890 | DIQMTQSPSSVSASV | 5243 | 3 | 3 |
| | | TCGGAGACAGAGTCACCATCACTTGTCGGGCGAGTCAGGGTAT | | GDRVTITCRASQGIS | | | |
| | | TAGCGGCTGGTTAGCCTGGTATCAGCAGAAACCAGGGAAAGCC | | GWLAWYQQKPGKAPK | | | |
| | | CCTAAGCTCCTCATCTATGCTGCAACCACTTTGCAAAGTGGGG | | LLIYAATTLQSGVPS | | | |
| | | TCCCATCAAGGTTCAGCGGCAGTGGATCTGGGACAGATTTCAC | | RFSGSGSGTDFTLTI | | | |
| | | TCTCACCATCAGCAGCCTGCAGCCTGAAGATTTTGCAACTTAC | | SSLQPEDFATYYCQQ | | | |
| | | TATTGCCAACAGGCTAACAGTTTCCCCCGGACGTTCGGCCAAG | | ANSFPRTFGQGTKVE | | | |
| | | GGACCAAGGTGGAGATCAAA | | IK | | | |
| ADI-81540 | 2947 | TCGTATGTGCTGACTCAGCCACCCTCAGTGTCAGTGGCCCCAG | 4891 | SYVLTQPPSVSVAPG | 5244 | 3 | 2 |
| | | GAAAGACGGCCAAGATTACCTGTGGGGGAAACAACATTGGAAG | | KTAKITCGGNNIGSK | | | |
| | | TAAACATGTGCACTGGTACCAGCAGAAGCCAGGCCAGGCCCCT | | HVHWYQQKPGQAPVL | | | |
| | | GTGCTGGTCATCTATTATGATAGCGACCGGCCCTCAGGGATCC | | VIYYDSDRPSGIPER | | | |
| | | CTGAGCGATTCTCTGGCTCCAACTCTGGGAACACGGCCACCCT | | FSGSNSGNTATLTIS | | | |
| | | GACCATCAGCAGGGTCGAAGCCGGGGATGAGGCCGACTATTAC | | RVEAGDEADYYCQVW | | | |
| | | TGTCAGGTGTGGGATAGTAATAGTGATCATCGTGTGTTCGGCG | | DSNSDHRVFGGGTKV | | | |
| | | GAGGGACCAAGGTCACCGTCCTA | | TVL | | | |
| ADI-81880 | 2936 | GAAATGACAATGACGCAGTCTCCAGGCACCCTGTCTTTGTCTC | 4892 | EMTMTQSPGTLSLSP | 5245 | 3 | 2 |
| | | CAGGGGAAAGAGCCACCCTCTCCTGCAGGGCCAGTCAGAGTGT | | GERATLSCRASQSVS | | | |
| | | TAGCAGCAGCTTTTTAGCCTGGTACCAGCACAAACCTGGCCAG | | SSFLAWYQHKPGQAP | | | |
| | | GCTCCCAGGCTCCTCATCTATGGTGCATCCAGCAGGGCCACTG | | RLLIYGASSRATGIP | | | |
| | | GCATCCCAGACAGGTTCAGTGGCAGTGGGTCTGGGACAGACTT | | DRFSGSGSGTDFTLT | | | |
| | | CACTCTCACCATCAGCAGACTGGAGCCTGAAGATTTTGCAGTG | | ISRLEPEDFAVYYCQ | | | |
| | | TATTACTGTCAGCAGTATGATAGCTCACCTCTCACTTTCGGCG | | QYDSSPLTFGGGTKV | | | |
| | | GAGGGACCAAAGTGGAGATCAAA | | EIK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81568 | IGKV1-9 | EIQMTQ | 3685 | RASQGI | 2154 | WYQQKP | 2257 | AASTLQ | 2392 | GVPARF | 4311 | QQLNSH | 4538 | FGQGTK |
| | | SPSVLS | | SSHLA | | GKAPKL | | S | | SGSGSG | | PCS | | VEIK |
| | | ASVGDR | | | | LIY | | | | TEFTLT | | | | |
| | | VTITC | | | | | | | | ISSLQP | | | | |
| | | | | | | | | | | EDFATY | | | | |
| | | | | | | | | | | YC | | | | |
| ADI-81658 | IGKV1D-39 | DIQMTQ | 1878 | RASQSI | 2066 | WYQQKP | 2257 | AASSLQ | 2391 | GVPSRF | 4312 | QQSSTT | 4539 | FGHGTR |
| | | SPSSLS | | STYLN | | GKAPKL | | S | | SGSGSG | | PIT | | LEIK |
| | | ASVGDR | | | | LIY | | | | TDFTLT | | | | |
| | | VTITC | | | | | | | | ISSLQP | | | | |
| | | | | | | | | | | EDFTTY | | | | |
| | | | | | | | | | | YC | | | | |
| ADI-81785 | IGKV1-NL1 | ETTLTQ | 1905 | RASQGI | 2062 | WYQQKP | 2259 | AASTLE | 2398 | GVPSRF | 2534 | QQYYTT | 2758 | FGQGTK |
| | | SPSSLS | | SNSLA | | GKAPKL | | S | | SGSGSG | | RT | | VEIK |
| | | ASVGDR | | | | LLY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81631 | IGKV1-33 | DIVMTQ | 1902 | QASQDI | 2148 | WYQQKP | 2257 | DASNLE | 2416 | GVPSRF | 4313 | HQYDNL | 2831 | FGQGTK |
| | | SPSSLS | | NNYLN | | GKAPKL | | T | | SGSGSG | | PRT | | VEIK |
| | | ASVGDR | | | | LIY | | | | TDFTFT | | | | |
| | | VTITC | | | | | | | | ISSLQP | | | | |
| | | | | | | | | | | EDSATY | | | | |
| | | | | | | | | | | YC | | | | |
| ADI-81841 | IGKV1-16 | EIVMTQ | 1891 | RASQGI | 3886 | WFQQKP | 2346 | GASSLQ | 2403 | GVPSKF | 4309 | QQYNDY | 4540 | FGQGTR |
| | | SPSSLS | | RNYLA | | GKAPKS | | S | | SGSGSG | | IPIT | | LEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81669 | IGKV2D-28 | EIAMTQ | 3686 | RSSQSL | 3887 | WYLLKP | 4018 | LGSNRA | 2387 | GVPDRF | 4314 | MQALQT | 2915 | FGGGTK |
| | | SPLSLP | | LHSNGY | | GQSPQL | | S | | SGSGSG | | PLT | | VEIK |
| | | VTPGEP | | NYLD | | LIY | | | | TDFSLK | | | | |
| | | ASISC | | | | | | | | ISRVEA | | | | |
| | | | | | | | | | | EDVGVY | | | | |
| | | | | | | | | | | YC | | | | |
| ADI-81787 | IGKV1D-39 | EIPMTQ | 1912 | RASQSI | 3888 | WYQQKP | 2257 | AASSLQ | 2391 | GVPSRF | 2534 | LQSYNT | 4541 | FGQGTK |
| | | SPSSLS | | NTYLN | | GKAPKL | | S | | SGSGSG | | PPWT | | VEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81788 | IGKV3D-15 | EMTLTQ | 1916 | RASQSV | 3889 | WYQQKP | 2258 | GASTRA | 2383 | GIPARF | 4315 | QQYNNL | 4542 | FGQGTK |
| | | SPATLS | | SSKLA | | GQAPRL | | T | | SGSGSG | | PPWT | | VEIK |
| | | VSPGER | | | | LIY | | | | TEFTLT | | | | |
| | | ATLSC | | | | | | | | ISSLQF | | | | |
| | | | | | | | | | | EDFAVY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81568 | 2937 | GAAATCCAAATGACCCAGTCTCCATCCGTCCTGTCTGCATCTG | 4893 | EIQMTQSPSVLSASV | 5246 | 3 | 3 |
| | | TAGGAGACAGAGTCACCATCACTTGCCGGGCCAGTCAGGGCAT | | GDRVTITCRASQGIS | | | |
| | | TAGCAGTCATTTAGCCTGGTATCAGCAAAAACCAGGGAAAGCC | | SHLAWYQQKPGKAPK | | | |
| | | CCTAAACTCCTGATCTATGCTGCATCCACTTTGCAAAGTGGGG | | LLIYAASTLQSGVPA | | | |
| | | TCCCAGCAAGGTTCAGCGGCAGTGGATCTGGGACAGAATTCAC | | RFSGSGSGTEFTLTI | | | |
| | | TCTCACGATCAGCAGCCTGCAGCCTGAAGATTTTGCAACTTAT | | SSLQPEDFATYYCQQ | | | |
| | | TACTGTCAACAGCTTAATAGTCACCCGTGCAGTTTTGGCCAGG | | LNSHPCSFGQGTKVE | | | |
| | | GGACCAAGGTGGAAATCAAA | | IK | | | |
| ADI-81658 | 4602 | GACATCCAGATGACCCAGTCTCCATCCTCCCTGTCTGCATCTG | 4894 | DIQMTQSPSSLSASV | 5247 | 2 | 2 |
| | | TAGGAGACAGAGTCACCATCACTTGCCGGGCAAGTCAGAGCAT | | GDRVTITCRASQSIS | | | |
| | | TAGCACCTATTTAAATTGGTATCAGCAGAAACCAGGGAAAGCC | | TYLNWYQQKPGKAPK | | | |
| | | CCTAAACTCCTGATCTATGCTGCATCCAGTTTGCAAAGTGGGG | | LLIYAASSLQSGVPS | | | |
| | | TCCCATCTAGGTTCAGTGGCAGTGGATCTGGGACAGATTTCAC | | RFSGSGSGTDFTLTI | | | |
| | | TCTCACCATCAGCAGTCTGCAACCTGAAGATTTTACAACTTAC | | SSLQPEDFTTYYCQQ | | | |
| | | TACTGTCAACAGAGTTCCACAACGCCGATCACCTTCGGCCACG | | SSTTPITFGHGTRLE | | | |
| | | GGACACGACTGGAGATTAAA | | IK | | | |
| ADI-81785 | 2937 | GAAACGACACTCACGCAGTCTCCATCCTCCCTGTCTGCATCTG | 4895 | ETTLTQSPSSLSASV | 5248 | 2 | 2 |
| | | TAGGAGACAGAGTCACCATCACTTGCCGGGCGAGTCAGGGCAT | | GDRVTITCRASQGIS | | | |
| | | TAGCAATTCTTTAGCCTGGTATCAGCAGAAACCAGGGAAAGCC | | NSLAWYQQKPGKAPK | | | |
| | | CCTAAGCTCCTGCTCTATGCTGCATCCACATTGGAAAGTGGGG | | LLLYAASTLESGVPS | | | |
| | | TCCCATCCAGGTTCAGTGGCAGTGGATCTGGGACGGATTTCAC | | RFSGSGSGTDFTLTI | | | |
| | | TCTCACCATCAGCAGCCTGCAGCCTGAAGATTTTGCAACTTAT | | SSLQPEDFATYYCQQ | | | |
| | | TACTGTCAACAGTATTATACTACTCGGACGTTCGGCCAAGGGA | | YYTTRTFGQGTKVEI | | | |
| | | CCAAGGTGGAAATCAAA | | K | | | |
| ADI-81631 | 2937 | GATATTGTGATGACCCAGTCTCCATCCTCCCTGTCTGCATCTG | 4896 | DIVMTQSPSSLSASV | 5249 | 2 | 2 |
| | | TAGGAGACAGAGTCACCATCACTTGCCAGGCGAGTCAGGACAT | | GDRVTITCQASQDIN | | | |
| | | TAACAACTATTTAAATTGGTATCAACAGAAACCAGGGAAAGCC | | NYLNWYQQKPGKAPK | | | |
| | | CCTAAGCTCCTGATCTACGATGCATCCAATTTGGAGACAGGGG | | LLIYDASNLETGVPS | | | |
| | | TCCCATCAAGGTTCAGTGGAAGTGGATCTGGGACAGACTTTAC | | RFSGSGSGTDFTFTI | | | |
| | | TTTCACCATCAGCAGCCTGCAGCCTGAAGATAGTGCAACATAT | | SSLQPEDSATYYCHQ | | | |
| | | TACTGTCACCAGTATGATAATCTCCCTAGGACGTTCGGCCAAG | | YDNLPRTFGQGTKVE | | | |
| | | GGACCAAGGTGGAAATCAAA | | IK | | | |
| ADI-81841 | 2940 | GAAATCGTGATGACCCAGTCTCCATCCTCACTGTCTGCATCTG | 4897 | EIVMTQSPSSLSASV | 5250 | 2 | 2 |
| | | TTGGAGACAGAGTCACCATCACTTGTCGGGCGAGTCAGGGCAT | | GDRVTITCRASQGIR | | | |
| | | TAGGAATTATTTAGCCTGGTTTCAGCAGAAACCAGGGAAAGCC | | NYLAWFQQKPGKAPK | | | |
| | | CCTAAGTCCCTGATCTATGGTGCATCCAGTTTGCAAAGCGGGG | | SLIYGASSLQSGVPS | | | |
| | | TCCCATCAAAGTTCAGCGGCAGTGGATCTGGGACAGATTTCAC | | KFSGSGSGTDFTLTI | | | |
| | | TCTCACCATCAGCAGCCTGCAGCCTGAAGACTTTGCAACTTAT | | SSLQPEDFATYYCQQ | | | |
| | | TACTGCCAACAGTATAATGATTACCCGATCACCTTCGGCCAAG | | YNDYPITFGQGTRLE | | | |
| | | GGACACGACTGGAGATTAAA | | IK | | | |
| ADI-81669 | 2936 | GAAATTGCGATGACCCAGTCTCCACTCTCCCTGCCCGTCACCC | 4898 | EIAMTQSPLSLPVTP | 5251 | 2 | 2 |
| | | CTGGAGAGCCGGCCTCCATCTCCTGCAGGTCTAGTCAGAGCCT | | GEPASISCRSSQSLL | | | |
| | | CCTGCATAGTAATGGATACAACTATTTGGATTGGTACCTGCTG | | HSNGYNYLDWYLLKP | | | |
| | | AAGCCAGGGCAGTCTCCACAACTCCTGATCTATTTGGGTTCTA | | GQSPQLLIYLGSNRA | | | |
| | | ATCGGGCCTCCGGGGTCCCTGACAGGTTCAGTGGCAGTGGATC | | SGVPDRFSGSGSGTD | | | |
| | | AGGCACAGATTTTTCACTGAAAATCAGCAGAGTGGAGGCTGAG | | FSLKISRVEAEDVGV | | | |
| | | GATGTTGGGGTTTATTACTGCATGCAAGCTCTACAAACTCCCC | | YYCMQALQTPLTFGG | | | |
| | | TCACTTTCGGCGGAGGGACCAAGGTGGAGATCAAA | | GTKVEIK | | | |
| ADI-81787 | 2937 | GAAATCCCAATGACGCAGTCTCCATCCTCCCTGTCTGCATCTG | 4899 | EIPMTQSPSSLSASV | 5252 | 2 | 2 |
| | | TTGGAGACAGAGTCACCATCACTTGCCGGGCAAGTCAGAGCAT | | GDRVTITCRASQSIN | | | |
| | | TAACACCTATTTAAATTGGTATCAGCAGAAACCAGGGAAAGCC | | TYLNWYQQKPGKAPK | | | |
| | | CCTAAACTCCTGATCTATGCTGCATCCAGTTTGCAAAGTGGGG | | LLIYAASSLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAACCTGAAGATTTTGCAACTTAC | | SSLQPEDFATYYCLQ | | | |
| | | TACTGTCTACAGAGTTACAATACCCCTCCGTGGACGTTCGGCC | | SYNTPPWTFGQGTKV | | | |
| | | AAGGGACCAAGGTGGAAATCAAA | | EIK | | | |
| ADI-81788 | 2937 | GAAATGACACTCACGCAGTCTCCAGCCACCCTGTCTGTGTCTC | 4900 | EMTLTQSPATLSVSP | 5253 | 2 | 2 |
| | | CGGGGGAAAGAGCCACCCTCTCCTGCAGGGCCAGTCAGAGTGT | | GERATLSCRASQSVS | | | |
| | | TAGCAGCAAATTAGCCTGGTACCAGCAGAAACCTGGCCAGGCT | | SKLAWYQQKPGQAPR | | | |
| | | CCCAGGCTCCTCATCTATGGTGCATCCACCAGGGCCACTGGTA | | LLIYGASTRATGIPA | | | |
| | | TCCCAGCCAGGTTCAGTGGCAGTGGGTCTGGGACAGAGTTCAC | | RFSGSGSGTEFTLTI | | | |
| | | TCTCACCATCAGCAGCCTGCAGTTTGAAGATTTTGCGGTTTAT | | SSLQFEDFAVYYCQQ | | | |
| | | TACTGTCAGCAGTATAATAACTTGCCTCCGTGGACATTCGGCC | | YNNLPPWTFGQGTKV | | | |
| | | AAGGGACCAAGGTGGAAATCAAA | | EIK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81566 | IGKV1-5 | DIQMTQ | 1879 | RASQSI | 3890 | WYQQKP | 2257 | DASTLE | 4147 | GVPSRF | 2532 | QQYKNY | 4543 | FGQGTK |
| | | SPSTLS | | NSWLA | | GKAPKL | | S | | SGSGSG | | WT | | VEIK |
| | | ASVGDR | | | | LIY | | | | TEFTLT | | | | |
| | | VTITC | | | | | | | | ISSLQP | | | | |
| | | | | | | | | | | DDFATY | | | | |
| | | | | | | | | | | YC | | | | |
| ADI-81662 | IGKV1-9 | DIRLTQ | 3687 | RASQGI | 2136 | WYQQKP | 2275 | AASTLQ | 2392 | GVPSRF | 2592 | QQLNSD | 4544 | FGPGTK |
| | | SPSFLS | | SSYLA | | GRAPKL | | S | | SGSGSG | | LFT | | VEIK |
| | | ASVGDS | | | | LIY | | | | TEFTLT | | | | |
| | | VTITC | | | | | | | | ISSLQP | | | | |
| | | | | | | | | | | EDFATY | | | | |
| | | | | | | | | | | YC | | | | |
| ADI-81694 | IGLV1-40 | QSVLTQ | 1948 | TGSSSN | 2201 | WYQQLP | 2314 | SNNNRP | 4073 | GVPDRF | 2599 | QSYDSS | 4545 | FGGGTK |
| | | PPSVSG | | IGAGYD | | GTAPKL | | S | | SGSKSG | | LSGYVV | | LTVL |
| | | APGQRV | | VH | | LIY | | | | TSASLA | | | | |
| | | TISC | | | | | | | | ITGLQA | | | | |
| | | | | | | | | | | EDEADY | | | | |
| | | | | | | | | | | YC | | | | |
| ADI-81674 | IGKV4-1 | DIPVTQ | 3688 | KSSQSV | 2254 | WYQQKP | 2293 | WASTRE | 2422 | GVPDRF | 4316 | QQYYNT | 4546 | FGGGTK |
| | | SPDSLA | | LYSSNN | | GQPPRL | | S | | SGSGSG | | PLT | | VEIK |
| | | VSLGER | | KNYLA | | LIY | | | | TDFTLT | | | | |
| | | ATINC | | | | | | | | IRSLQA | | | | |
| | | | | | | | | | | EDVAVY | | | | |
| | | | | | | | | | | YC | | | | |
| ADI-81675 | IGKV4-1 | DIQVTQ | 3689 | KSSQSV | 2254 | WYQQKP | 2293 | WASTRE | 2422 | GVPDRF | 4316 | QQYYNT | 4546 | FGGGTK |
| | | SPDSLA | | LYSSNN | | GQPPRL | | S | | SGSGSG | | PLT | | VDIK |
| | | VSLGER | | KNYLA | | LIY | | | | TDFTLT | | | | |
| | | ATINC | | | | | | | | IRSLQA | | | | |
| | | | | | | | | | | EDVAVY | | | | |
| | | | | | | | | | | YC | | | | |
| ADI-81878 | IGLV3-1 | SYVLTQ | 3690 | SGDKLG | 2222 | WYQQKP | 4019 | QDTKRP | 4148 | GIPERF | 4317 | QAWDSS | 4547 | FGAGTK |
| | | PPSVSV | | DKYAC | | GQSPVL | | S | | SGSNSG | | NHYV | | VTVL |
| | | SPGQTA | | | | VIY | | | | NTATLT | | | | |
| | | SITC | | | | | | | | ISGTQP | | | | |
| | | | | | | | | | | MDEADY | | | | |
| | | | | | | | | | | YC | | | | |
| ADI-81619 | IGLV1-44 | QYVLTQ | 3691 | SGSSSN | 2217 | WYQQLP | 2314 | NNNQRP | 4061 | GVPDRF | 2615 | AAWDDS | 4548 | FGGGTK |
| | | PPSASG | | IGSNTV | | GTAPKL | | S | | SGSKSG | | LNGVL | | LTVL |
| | | TPVQRV | | N | | LIY | | | | TSASLA | | | | |
| | | TISC | | | | | | | | ISGLQS | | | | |
| | | | | | | | | | | EDEADY | | | | |
| | | | | | | | | | | YC | | | | |
| ADI-81744 | IGKV1-9 | DIRMTQ | 3683 | RASQGI | 2187 | WYQHKP | 3924 | AASTLQ | 2392 | GVPSRF | 2592 | QQLNSY | 4549 | FGQGTK |
| | | SPSFLS | | SNYLA | | GKAPKL | | S | | SGSGSG | | PRT | | VDIK |
| | | ASVGDR | | | | LIY | | | | TEFTLT | | | | |
| | | VTITC | | | | | | | | ISSLQP | | | | |
| | | | | | | | | | | EDFATY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81566 | 2937 | GACATCCAGATGACCCAGTCTCCTTCCACCCTGTCTGCATCTG | 4901 | DIQMTQSPSTLSASV | 5254 | 2 | 2 |
| | | TTGGAGACAGAGTCACCATCACTTGCCGGGCCAGTCAGAGTAT | | GDRVTITCRASQSIN | | | |
| | | TAATAGCTGGTTGGCCTGGTATCAGCAGAAACCAGGGAAAGCC | | SWLAWYQQKPGKAPK | | | |
| | | CCTAAACTCCTGATCTATGATGCCTCCACTTTGGAAAGTGGGG | | LLIYDASTLESGVPS | | | |
| | | TCCCATCAAGGTTCAGCGGCAGTGGATCTGGGACAGAATTCAC | | RFSGSGSGTEFTLTI | | | |
| | | TCTCACCATCAGCAGCCTGCAGCCTGATGATTTCGCAACTTAT | | SSLQPDDFATYYCQQ | | | |
| | | TACTGCCAACAGTATAAAAATTATTGGACGTTCGGCCAAGGGA | | YKNYWTFGQGTKVEI | | | |
| | | CCAAGGTGGAAATCAAA | | K | | | |
| ADI-81662 | 2942 | GACATCCGGTTGACCCAGTCTCCGTCCTTCCTGTCTGCATCTG | 4902 | DIRLTQSPSFLSASV | 5255 | 2 | 2 |
| | | TAGGAGACAGCGTCACCATCACTTGCCGGGCCAGTCAGGGCAT | | GDSVTITCRASQGIS | | | |
| | | TAGCAGTTATTTAGCCTGGTATCAGCAAAAACCAGGGAGAGCC | | SYLAWYQQKPGRAPK | | | |
| | | CCTAAGCTCCTGATCTATGCTGCATCCACTTTGCAAAGTGGAG | | LLIYAASTLQSGVPS | | | |
| | | TCCCATCAAGGTTCAGCGGCAGTGGATCTGGGACAGAATTCAC | | RFSGSGSGTEFTLTI | | | |
| | | TCTCACAATCAGCAGCCTGCAGCCTGAAGATTTTGCTACTTAT | | SSLQPEDFATYYCQQ | | | |
| | | TACTGTCAACAACTTAATAGTGACCTCTTCACTTTCGGCCCTG | | LNSDLFTFGPGTKVE | | | |
| | | GGACCAAGGTGGAGATCAAA | | IK | | | |
| ADI-81694 | 2939 | CAGTCTGTGCTGACGCAGCCGCCCTCAGTGTCTGGGGCCCCAG | 4903 | QSVLTQPPSVSGAPG | 5256 | 2 | 2 |
| | | GGCAGAGGGTCACCATCTCCTGCACTGGGAGCAGCTCCAACAT | | QRVTISCTGSSSNIG | | | |
| | | CGGGGCAGGTTATGATGTACATTGGTACCAGCAACTTCCAGGA | | AGYDVHWYQQLPGTA | | | |
| | | ACAGCCCCCAAACTCCTCATCTATAGTAACAACAATCGGCCCT | | PKLLIYSNNNRPSGV | | | |
| | | CAGGGGTCCCTGACCGATTCTCTGGCTCCAAGTCTGGCACCTC | | PDRFSGSKSGTSASL | | | |
| | | AGCCTCCCTGGCCATCACTGGACTCCAGGCTGAGGATGAGGCT | | AITGLQAEDEADYYC | | | |
| | | GATTATTACTGCCAGTCCTATGACAGCAGCCTGAGTGGCTATG | | QSYDSSLSGYVVFGG | | | |
| | | TGGTATTCGGCGGAGGGACCAAGCTCACCGTCCTA | | GTKLTVL | | | |
| ADI-81674 | 2936 | GACATCCCGGTGACCCAGTCTCCAGACTCCCTGGCTGTGTCTC | 4904 | DIPVTQSPDSLAVSL | 5257 | 2 | 2 |
| | | TGGGCGAGAGGGCCACCATCAACTGCAAGTCCAGCCAGAGTGT | | GERATINCKSSQSVL | | | |
| | | TTTATACAGCTCCAACAATAAGAACTACTTAGCTTGGTACCAG | | YSSNNKNYLAWYQQK | | | |
| | | CAGAAACCAGGACAGCCTCCTAGGCTGCTCATTTACTGGGCAT | | PGQPPRLLIYWASTR | | | |
| | | CTACCCGGGAATCCGGGGTCCCTGACCGATTCAGTGGCAGCGG | | ESGVPDRFSGSGSGT | | | |
| | | GTCTGGGACAGATTTCACTCTCACCATCCGCAGCCTGCAGGCT | | DFTLTIRSLQAEDVA | | | |
| | | GAAGATGTGGCAGTTTATTACTGTCAGCAATATTATAATACCC | | VYYCQQYYNTPLTFG | | | |
| | | CGCTCACTTTCGGCGGAGGGACCAAGGTGGAAATCAAA | | GGTKVEIK | | | |
| ADI-81675 | 2938 | GACATCCAGGTGACCCAGTCTCCAGACTCCCTGGCTGTGTCTC | 4905 | DIQVTQSPDSLAVSL | 5258 | 2 | 2 |
| | | TGGGCGAGAGGGCCACCATCAACTGCAAGTCCAGCCAGAGTGT | | GERATINCKSSQSVL | | | |
| | | TTTATACAGCTCCAACAATAAGAACTACTTAGCTTGGTACCAG | | YSSNNKNYLAWYQQK | | | |
| | | CAGAAACCAGGACAGCCTCCTAGGCTGCTCATTTACTGGGCAT | | PGQPPRLLIYWASTR | | | |
| | | CTACCCGGGAATCCGGGGTCCCTGACCGATTCAGTGGCAGCGG | | ESGVPDRFSGSGSGT | | | |
| | | GTCTGGGACAGATTTCACTCTCACCATCCGCAGCCTGCAGGCT | | DFTLTIRSLQAEDVA | | | |
| | | GAAGATGTGGCAGTTTATTACTGTCAGCAATATTATAATACCC | | VYYCQQYYNTPLTFG | | | |
| | | CGCTCACTTTCGGCGGAGGGACCAAAGTGGATATCAAA | | GGTKVDIK | | | |
| ADI-81878 | 4603 | TCGTATGTGCTGACACAGCCACCCTCAGTGTCCGTGTCCCCAG | 4906 | SYVLTQPPSVSVSPG | 5259 | 2 | 2 |
| | | GACAGACAGCCAGCATCACCTGCTCTGGAGATAAATTGGGGGA | | QTASITCSGDKLGDK | | | |
| | | TAAATATGCTTGCTGGTATCAGCAGAAGCCAGGCCAGTCCCCT | | YACWYQQKPGQSPVL | | | |
| | | GTACTGGTCATCTATCAAGATACCAAGCGGCCCTCAGGGATCC | | VIYQDTKRPSGIPER | | | |
| | | CTGAGCGATTCTCTGGCTCCAACTCTGGGAACACAGCCACTCT | | FSGSNSGNTATLTIS | | | |
| | | GACCATCAGCGGGACCCAGCCTATGGATGAGGCTGACTATTAC | | GTQPMDEADYYCQAW | | | |
| | | TGTCAGGCGTGGGACAGCAGTAATCATTATGTCTTCGGAGCTG | | DSSNHYVFGAGTKVT | | | |
| | | GGACCAAGGTCACCGTCCTA | | VL | | | |
| ADI-81619 | 2939 | CAGTATGTGCTGACTCAGCCACCCTCAGCGTCTGGGACCCCCG | 4907 | QYVLTQPPSASGTPV | 5260 | 2 | 2 |
| | | TGCAGAGGGTCACCATCTCTTGTTCTGGAAGCAGCTCCAACAT | | QRVTISCSGSSSNIG | | | |
| | | CGGAAGTAATACTGTTAACTGGTACCAGCAGCTCCCAGGAACG | | SNTVNWYQQLPGTAP | | | |
| | | GCCCCCAAACTCCTCATCTATAATAATAATCAGCGGCCCTCAG | | KLLIYNNNQRPSGVP | | | |
| | | GGGTCCCTGACCGATTCTCTGGCTCCAAGTCTGGCACCTCAGC | | DRFSGSKSGTSASLA | | | |
| | | CTCCCTGGCCATCAGTGGGCTCCAGTCTGAGGATGAGGCTGAT | | ISGLQSEDEADYYCA | | | |
| | | TATTACTGTGCAGCATGGGATGACAGCCTGAATGGTGTGCTAT | | AWDDSLNGVLFGGGT | | | |
| | | TCGGCGGAGGGACCAAGCTCACCGTCCTA | | KLTVL | | | |
| ADI-81744 | 2944 | GACATCCGGATGACCCAGTCTCCATCCTTCCTGTCTGCATCTG | 4908 | DIRMTQSPSFLSASV | 5261 | 2 | 2 |
| | | TAGGAGACAGAGTCACCATCACTTGCCGGGCCAGTCAGGGCAT | | GDRVTITCRASQGIS | | | |
| | | TAGCAATTATTTAGCCTGGTATCAGCACAAACCAGGGAAAGCC | | NYLAWYQHKPGKAPK | | | |
| | | CCTAAGCTCCTGATCTATGCTGCATCCACTTTGCAAAGTGGGG | | LLIYAASTLQSGVPS | | | |
| | | TCCCATCAAGGTTCAGCGGCAGTGGATCTGGGACAGAATTCAC | | RFSGSGSGTEFTLTI | | | |
| | | TCTCACAATCAGCAGCCTGCAGCCTGAAGATTTTGCAACTTAT | | SSLQPEDFATYYCQQ | | | |
| | | TACTGTCAACAGCTTAATAGTTACCCTCGGACGTTCGGCCAAG | | LNSYPRTFGQGTKVD | | | |
| | | GGACCAAGGTGGATATCAAA | | IK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81633 | IGLV6-57 | KFMLTQ | 3692 | TGSSGY | 3891 | WYQQRP | 2298 | EDNQRP | 2431 | GVPDRF | 2574 | QSYDNT | 4550 | FGGGTK |
| | | PHSVSE | | IASNYV | | GSAPTT | | S | | SGSIDS | | NWV | | LTVL |
| | | SPGKTV | | IQ | | VIY | | | | SSNSAS | | | | |
| | | TISC | | | | | | | | LTISGL | | | | |
| | | | | | | | | | | KTEDEA | | | | |
| | | | | | | | | | | DYYC | | | | |
| ADI-81700 | IGKV1-9 | DIVMTQ | 3693 | RASQGI | 2136 | WYQQKP | 2257 | AASTLQ | 2392 | GVPLRF | 2595 | QQLNSY | 4551 | FGQGTK |
| | | SPSLLS | | SSYLA | | GKAPKL | | S | | SGSGSG | | PRYT | | VDIK |
| | | ASVGDR | | | | LIY | | | | TEFTLT | | | | |
| | | VTITC | | | | | | | | ISSLQP | | | | |
| | | | | | | | | | | EDFATY | | | | |
| | | | | | | | | | | YC | | | | |
| ADI-81797 | IGKV1-5 | EIVMTQ | 3694 | RASQSI | 2190 | WYQQKP | 2257 | KASSLE | 2386 | GVPSRF | 2532 | QQYNSY | 4552 | FGPGTK |
| | | SPSTLS | | SSWLA | | GKAPKL | | S | | SGSGSG | | FT | | VEIK |
| | | ASAGDR | | | | LIY | | | | TEFTLT | | | | |
| | | VTITC | | | | | | | | ISSLQP | | | | |
| | | | | | | | | | | DDFATY | | | | |
| | | | | | | | | | | YC | | | | |
| ADI-81854 | IGKV3D-15 | ETTLTQ | 2022 | RASQSV | 2227 | WYQQKP | 3906 | GASTRA | 2383 | GIPARF | 4288 | QQYDNW | 4553 | FGQGTK |
| | | SPATLS | | SSNLA | | GQAPRV | | T | | SGSGSG | | PPMWT | | VEIK |
| | | VSPGER | | | | LIY | | | | TEFTLT | | | | |
| | | ATLSC | | | | | | | | ISSLQS | | | | |
| | | | | | | | | | | EDSAVY | | | | |
| | | | | | | | | | | YC | | | | |
| ADI-81866 | IGKV3D-15 | EIVMTQ | 2034 | RASQSI | 2129 | WYQQKP | 3996 | GASTRA | 2383 | GIPARF | 2569 | QQHNSW | 4554 | FGQGTR |
| | | SPATLS | | SSNLA | | GQAPRL | | T | | SGSGSG | | PAT | | LEIK |
| | | VSPGER | | | | LIS | | | | TEFTLT | | | | |
| | | ATLSC | | | | | | | | ISSLQS | | | | |
| | | | | | | | | | | EDFAVY | | | | |
| | | | | | | | | | | YC | | | | |
| ADI-81903 | IGKV1D-39 | EMTLTQ | 1914 | RASQSI | 2050 | WYQQKL | 4020 | AASSLQ | 2391 | GVPSRF | 2534 | QQSYST | 4555 | FGQGTK |
| | | SPSSLS | | SSYLN | | GKAPKL | | S | | SGSGSG | | PYT | | VDIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81703 | IGKV1-5 | EIRMTQ | 3695 | RASQSI | 2190 | WYQQKP | 2263 | KASILE | 4149 | GVPSRF | 2532 | QQYDSY | 4556 | FGQGTK |
| | | SPSTLS | | SSWLA | | GKAPNL | | S | | SGSGSG | | SGT | | VEIK |
| | | ASVGDR | | | | LIY | | | | TEFTLT | | | | |
| | | VTITC | | | | | | | | ISSLQP | | | | |
| | | | | | | | | | | DDFATY | | | | |
| | | | | | | | | | | YC | | | | |
| ADI-81739 | IGLV1-40 | QSVLTQ | 1948 | TGSSSN | 3892 | WYQQVP | 4021 | GNSNRP | 2477 | GVPDRF | 2599 | QSYDSS | 4557 | FGGGTK |
| | | PPSVSG | | IGAGFD | | GTAPKL | | S | | SGSKSG | | LSGFVI | | LTVL |
| | | APGQRV | | VH | | LIY | | | | TSASLA | | | | |
| | | TISC | | | | | | | | ITGLQA | | | | |
| | | | | | | | | | | EDEADY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81633 | 2939 | AAGTTTATGCTGACTCAGCCCCACTCTGTGTCGGAGTCTCCGG | 4909 | KFMLTQPHSVSESPG | 5262 | 2 | 1 |
| | | GGAAGACGGTAACCATCTCCTGCACCGGCAGCAGTGGCTACAT | | KTVTISCTGSSGYIA | | | |
| | | TGCCAGCAACTATGTGCAGTGGTACCAGCAGCGCCCGGGCAGT | | SNYVQWYQQRPGSAP | | | |
| | | GCCCCCACCACTGTGATCTATGAGGATAACCAAAGACCCTCTG | | TTVIYEDNQRPSGVP | | | |
| | | GGGTCCCTGATCGGTTCTCTGGCTCCATCGACAGCTCCTCCAA | | DRFSGSIDSSSNSAS | | | |
| | | TTCTGCCTCCCTCACCATCTCTGGACTGAAGACTGAGGACGAG | | LTISGLKTEDEADYY | | | |
| | | GCTGACTACTACTGTCAGTCTTATGATAACACCAATTGGGTGT | | CQSYDNTNWVFGGGT | | | |
| | | TCGGCGGAGGGACCAAGCTCACCGTCCTA | | KLTVL | | | |
| ADI-81700 | 2944 | GATATTGTGATGACGCAGTCTCCATCCCTCCTGTCTGCATCTG | 4910 | DIVMTQSPSLLSASV | 5263 | 2 | 2 |
| | | TAGGAGACAGAGTCACCATCACTTGCCGGGCCAGTCAGGGCAT | | GDRVTITCRASQGIS | | | |
| | | TAGCAGTTATTTAGCCTGGTATCAGCAAAAACCAGGGAAAGCC | | SYLAWYQQKPGKAPK | | | |
| | | CCTAAGCTCCTGATCTATGCTGCATCCACTTTGCAAAGTGGGG | | LLIYAASTLQSGVPL | | | |
| | | TCCCATTAAGGTTCAGCGGCAGTGGATCTGGGACAGAATTCAC | | RFSGSGSGTEFTLTI | | | |
| | | TCTCACAATCAGCAGCCTGCAGCCTGAAGATTTTGCAACTTAT | | SSLQPEDFATYYCQQ | | | |
| | | TACTGTCAACAGCTTAATAGTTACCCTCGGTACACTTTTGGCC | | LNSYPRYTFGQGTKV | | | |
| | | AGGGGACCAAGGTGGATATCAAA | | DIK | | | |
| ADI-81797 | 2942 | GAAATCGTGATGACCCAGTCTCCTTCCACCCTGTCTGCATCTG | 4911 | EIVMTQSPSTLSASA | 5264 | 2 | 1 |
| | | CTGGAGACAGAGTCACCATCACTTGCCGGGCCAGTCAGAGTAT | | GDRVTITCRASQSIS | | | |
| | | TAGTAGCTGGTTGGCCTGGTATCAGCAGAAACCAGGAAAAGCC | | SWLAWYQQKPGKAPK | | | |
| | | CCTAAACTCCTGATCTATAAGGCGTCTAGTTTAGAAAGTGGGG | | LLIYKASSLESGVPS | | | |
| | | TCCCATCAAGGTTCAGCGGCAGTGGATCTGGGACAGAATTCAC | | RFSGSGSGTEFTLTI | | | |
| | | TCTCACCATCAGCAGCCTGCAGCCTGATGATTTTGCAACTTAT | | SSLQPDDFATYYCQQ | | | |
| | | TACTGCCAACAGTATAATAGTTATTTCACTTTCGGCCCTGGGA | | YNSYFTFGPGTKVEI | | | |
| | | CCAAGGTGGAAATCAAA | | K | | | |
| ADI-81854 | 2937 | GAAACGACACTCACGCAGTCTCCAGCCACCCTGTCTGTGTCTC | 4912 | ETTLTQSPATLSVSP | 5265 | 2 | 2 |
| | | CAGGGGAAAGAGCCACCCTCTCCTGCAGGGCCAGTCAGAGTGT | | GERATLSCRASQSVS | | | |
| | | TAGCAGCAACTTAGCCTGGTACCAGCAGAAGCCTGGCCAGGCT | | SNLAWYQQKPGQAPR | | | |
| | | CCCAGGGTCCTCATCTATGGTGCATCCACCAGGGCCACTGGTA | | VLIYGASTRATGIPA | | | |
| | | TCCCAGCCAGGTTCAGTGGCAGTGGGTCTGGGACAGAGTTCAC | | RFSGSGSGTEFTLTI | | | |
| | | TCTCACCATCAGCAGCCTGCAGTCTGAAGATTCTGCAGTTTAT | | SSLQSEDSAVYYCQQ | | | |
| | | TACTGTCAGCAGTATGATAACTGGCCTCCCATGTGGACGTTCG | | YDNWPPMWTFGQGTK | | | |
| | | GCCAAGGGACCAAGGTGGAAATCAAA | | VEIK | | | |
| ADI-81866 | 2940 | GAAATTGTGATGACGCAGTCTCCAGCCACCCTGTCTGTGTCTC | 4913 | EIVMTQSPATLSVSP | 5266 | 2 | 2 |
| | | CGGGGGAAAGAGCCACCCTCTCCTGCAGGGCCAGTCAGAGTAT | | GERATLSCRASQSIS | | | |
| | | TAGCAGCAACCTAGCCTGGTACCAGCAGAAACCTGGCCAGGCT | | SNLAWYQQKPGQAPR | | | |
| | | CCCAGGCTCCTCATCTCTGGTGCCTCCACCAGGGCCACTGGTA | | LLISGASTRATGIPA | | | |
| | | TCCCAGCCAGGTTCAGTGGCAGTGGGTCTGGGACAGAGTTCAC | | RFSGSGSGTEFTLTI | | | |
| | | TCTCACCATCAGCAGCCTGCAGTCTGAAGATTTTGCAGTTTAT | | SSLQSEDFAVYYCQQ | | | |
| | | TACTGTCAGCAGCATAATTCCTGGCCGGCCACCTTCGGCCAAG | | HNSWPATFGQGTRLE | | | |
| | | GGACACGACTGGAGATTAAA | | IK | | | |
| ADI-81903 | 2944 | GAAATGACGCTGACGCAGTCTCCATCCTCCCTGTCTGCATCTG | 4914 | EMTLTQSPSSLSASV | 5267 | 2 | 1 |
| | | TAGGAGACAGAGTCACCATCACTTGCCGGGCAAGTCAGAGCAT | | GDRVTITCRASQSIS | | | |
| | | TAGCAGTTATTTAAATTGGTATCAGCAAAAACTGGGGAAAGCC | | SYLNWYQQKLGKAPK | | | |
| | | CCTAAGCTCCTGATCTACGCTGCATCCAGTTTGCAAAGTGGGG | | LLIYAASSLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAACCTGAAGATTTTGCAACTTAC | | SSLQPEDFATYYCQQ | | | |
| | | TACTGTCAACAGAGTTACAGTACCCCGTACACTTTTGGCCAGG | | SYSTPYTFGQGTKVD | | | |
| | | GGACCAAAGTGGATATCAAA | | IK | | | |
| ADI-81703 | 2937 | GAAATCCGAATGACCCAGTCTCCTTCTACCCTGTCTGCATCTG | 4915 | EIRMTQSPSTLSASV | 5268 | 2 | 2 |
| | | TAGGAGACAGAGTCACCATCACTTGCCGGGCCAGTCAGAGTAT | | GDRVTITCRASQSIS | | | |
| | | TAGTAGCTGGTTGGCCTGGTATCAGCAGAAACCGGGGAAAGCC | | SWLAWYQQKPGKAPN | | | |
| | | CCTAATCTCCTGATCTATAAGGCGTCTATTTTAGAAAGTGGGG | | LLIYKASILESGVPS | | | |
| | | TCCCATCAAGGTTCAGCGGCAGTGGATCTGGGACAGAATTCAC | | RFSGSGSGTEFTLTI | | | |
| | | TCTCACCATCAGCAGCCTGCAGCCTGATGATTTTGCAACTTAT | | SSLQPDDFATYYCQQ | | | |
| | | TACTGCCAACAGTATGACAGTTATTCAGGGACGTTCGGCCAAG | | YDSYSGTFGQGTKVE | | | |
| | | GGACCAAGGTGGAAATCAAA | | IK | | | |
| ADI-81739 | 2939 | CAGTCTGTGTTGACGCAGCCGCCCTCAGTGTCTGGGGCCCCAG | 4916 | QSVLTQPPSVSGAPG | 5269 | 2 | 2 |
| | | GGCAGAGGGTCACCATCTCCTGCACTGGGAGCAGCTCCAACAT | | QRVTISCTGSSSNIG | | | |
| | | CGGGGCAGGTTTTGATGTACATTGGTACCAGCAGGTTCCAGGA | | AGFDVHWYQQVPGTA | | | |
| | | ACAGCCCCCAAACTCCTCATCTATGGTAACAGCAATCGGCCCT | | PKLLIYGNSNRPSGV | | | |
| | | CAGGCGTCCCTGACCGATTCTCTGGCTCCAAGTCCGGCACCTC | | PDRFSGSKSGTSASL | | | |
| | | AGCCTCCCTGGCCATCACTGGGCTCCAGGCTGAGGATGAGGCT | | AITGLQAEDEADYYC | | | |
| | | GATTATTACTGCCAGTCCTATGACAGCAGCCTGAGTGGCTTTG | | QSYDSSLSGFVIFGG | | | |
| | | TCATATTCGGCGGAGGCACCAAGCTCACCGTCCTA | | GTKLTVL | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81837 | IGKV1D-39 | EITMTQ | 3637 | RASQSI | 2066 | WYQQKP | 2257 | AASSLQ | 2391 | GVPSRF | 2606 | QQSYSN | 4558 | FGQGTK |
| | | SPSSLS | | STYLN | | GKAPKL | | S | | SGSGSG | | PMYT | | VEIK |
| | | ASVGDR | | | | LIY | | | | TDFTLT | | | | |
| | | VTITC | | | | | | | | ISSLQP | | | | |
| | | | | | | | | | | EDFASY | | | | |
| | | | | | | | | | | YC | | | | |
| ADI-81907 | IGKV3D-15 | EMTLTQ | 3626 | RASQSV | 3889 | WYQQKP | 2258 | GASTRA | 2383 | GIPARF | 2569 | QQYNNW | 4559 | FGGGTK |
| | | SPGTLS | | SSKLA | | GQAPRL | | T | | SGSGSG | | PPLT | | VEIK |
| | | VSPGER | | | | LIY | | | | TEFTLT | | | | |
| | | ATLSC | | | | | | | | ISSLQS | | | | |
| | | | | | | | | | | EDFAVY | | | | |
| | | | | | | | | | | YC | | | | |
| ADI-81654 | IGLV2-14 | QSVLTQ | 1918 | TGTSSD | 3893 | WYQQHP | 2265 | EVSNRP | 2427 | GVSNRF | 4318 | SSYASS | 4560 | FGGVTK |
| | | PASVSG | | VGGHNY | | GKAPKL | | S | | SGSKSG | | TSPVV | | LTVL |
| | | SPGQSI | | VS | | MIY | | | | NTASLT | | | | |
| | | TISC | | | | | | | | ISGLQA | | | | |
| | | | | | | | | | | EDEADY | | | | |
| | | | | | | | | | | SC | | | | |
| ADI-81677 | IGKV1-5 | DIRMTQ | 3610 | RASQSI | 2146 | WYQQKP | 2257 | KASSLE | 2386 | GDPSRF | 4319 | QQYSTY | 4561 | FGQGTK |
| | | SPSTLS | | STWLA | | GKAPKL | | S | | SGSGSG | | PYT | | VEIK |
| | | ASVGDR | | | | LIY | | | | TEFTLT | | | | |
| | | VTITC | | | | | | | | ISSLQP | | | | |
| | | | | | | | | | | DDFATY | | | | |
| | | | | | | | | | | YC | | | | |
| ADI-81790 | IGLV6-57 | NFMLTQ | 1992 | TRSSGS | 3894 | WYQQRP | 4009 | DDNQRP | 4150 | GVPDRF | 2574 | QSHDSS | 4562 | FGGGTK |
| | | PHSVSE | | IASSYV | | GSSPTT | | S | | SGSIDS | | TVI | | LTVL |
| | | SPGKTV | | Q | | VIY | | | | SSNSAS | | | | |
| | | TISC | | | | | | | | LTISGL | | | | |
| | | | | | | | | | | KTEDEA | | | | |
| | | | | | | | | | | DYYC | | | | |
| ADI-81755 | IGKV1-33 | EMTLTQ | 1914 | QASQAI | 3895 | WYQQKP | 2257 | DASNLE | 2416 | GVPSRF | 2542 | QQYDNL | 4563 | FGPGTK |
| | | SPSSLS | | SNYLN | | GKAPKL | | T | | SGSGSG | | PFT | | VDIK |
| | | ASVGDR | | | | ILIY | | | | TDFTFT | | | | |
| | | VTITC | | | | | | | | ISSLQP | | | | |
| | | | | | | | | | | EDIATY | | | | |
| | | | | | | | | | | FC | | | | |
| ADI-81893 | IGKV1D-39 | EIGVTQ | 3696 | RASQSI | 2050 | WYQQKP | 2257 | AASSLQ | 2391 | GVPSRF | 4320 | QQSYRT | 2891 | FGPGTK |
| | | SPSSLS | | SSYLN | | GKAPKL | | S | | SGSGFG | | PPLT | | VEIK |
| | | ASVGDR | | | | LIY | | | | TDFTFT | | | | |
| | | VTITC | | | | | | | | ISSLQP | | | | |
| | | | | | | | | | | EDFATY | | | | |
| | | | | | | | | | | YC | | | | |
| ADI-81649 | IGKV4-1 | DIVLTQ | 3697 | KSSQSV | 3896 | WYQQKP | 2290 | WASTRE | 2422 | GVPDRF | 2619 | QQYYST | 4518 | FGPGTK |
| | | SPDSLA | | LYSSNN | | GQPPKL | | S | | SGSGSG | | PLT | | VEIK |
| | | VSLGER | | ENYLG | | LIY | | | | TDFTLT | | | | |
| | | ATINC | | | | | | | | ISSLQA | | | | |
| | | | | | | | | | | EDVAVY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81837 | 2937 | GAAATCACAATGACCCAGTCTCCATCCTCCCTGTCTGCATCTG | 4917 | EITMTQSPSSLSASV | 5270 | 2 | 2 |
| | | TAGGAGACAGAGTCACCATCACTTGCCGGGCAAGTCAGAGCAT | | GDRVTITCRASQSIS | | | |
| | | TAGCACCTATTTAAACTGGTATCAGCAGAAACCAGGGAAAGCC | | TYLNWYQQKPGKAPK | | | |
| | | CCTAAGCTCCTGATCTATGCTGCATCCAGTTTGCAAAGTGGGG | | LLIYAASSLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAACCTGAAGATTTTGCAAGTTAC | | SSLQPEDFASYYCQQ | | | |
| | | TATTGTCAACAGAGTTACAGTAACCCCATGTACACTTTTGGCC | | SYSNPMYTFGQGTKV | | | |
| | | AGGGGACCAAGGTGGAAATCAAA | | EIK | | | |
| ADI-81907 | 2936 | GAAATGACACTCACGCAGTCTCCAGGCACCCTGTCTGTGTCTC | 4918 | EMTLTQSPGTLSVSP | 5271 | 2 | 2 |
| | | CAGGGGAAAGAGCCACCCTCTCCTGCAGGGCCAGTCAGAGTGT | | GERATLSCRASQSVS | | | |
| | | TAGCAGCAAGTTAGCCTGGTACCAGCAGAAACCTGGCCAGGCT | | SKLAWYQQKPGQAPR | | | |
| | | CCCAGGCTCCTCATCTATGGTGCATCCACCAGGGCCACTGGTA | | LLIYGASTRATGIPA | | | |
| | | TCCCAGCCAGGTTCAGTGGCAGTGGGTCTGGGACAGAGTTCAC | | RFSGSGSGTEFTLTI | | | |
| | | TCTCACCATCAGCAGCCTGCAGTCTGAAGATTTTGCAGTTTAT | | SSLQSEDFAVYYCQQ | | | |
| | | TACTGTCAGCAGTATAATAACTGGCCTCCGCTCACTTTCGGCG | | YNNWPPLTFGGGTKV | | | |
| | | GAGGGACCAAGGTGGAGATCAAA | | EIK | | | |
| ADI-81654 | 4604 | CAGTCTGTGCTGACTCAGCCTGCCTCCGTGTCTGGGTCTCCTG | 4919 | QSVLTQPASVSGSPG | 5272 | 2 | 2 |
| | | GACAGTCGATCACCATCTCCTGCACTGGAACCAGCAGTGACGT | | QSITISCTGTSSDVG | | | |
| | | TGGTGGTCATAACTATGTCTCCTGGTACCAACAGCACCCAGGC | | GHNYVSWYQQHPGKA | | | |
| | | AAAGCCCCCAAACTCATGATTTATGAGGTCAGTAATCGGCCCT | | PKLMIYEVSNRPSGV | | | |
| | | CAGGGGTTTCTAATCGCTTCTCTGGCTCCAAGTCTGGCAACAC | | SNRFSGSKSGNTASL | | | |
| | | GGCCTCCCTGACCATCTCTGGGCTCCAGGCTGAGGACGAGGCT | | TISGLQAEDEADYSC | | | |
| | | GATTATTCCTGCAGTTCATATGCAAGCAGTACCTCTCCTGTGG | | SSYASSTSPVVFGGV | | | |
| | | TTTTCGGCGGAGTGACCAAGCTCACCGTCCTA | | TKLTVL | | | |
| ADI-81677 | 2937 | GACATCCGGATGACCCAGTCTCCTTCCACCCTGTCTGCATCTG | 4920 | DIRMTQSPSTLSASV | 5273 | 2 | 2 |
| | | TAGGAGACAGAGTCACCATCACTTGCCGGGCCAGTCAGAGTAT | | GDRVTITCRASQSIS | | | |
| | | TAGTACCTGGTTGGCCTGGTATCAGCAGAAACCAGGGAAAGCC | | TWLAWYQQKPGKAPK | | | |
| | | CCTAAGCTCCTGATCTATAAGGCGTCTAGTTTAGAAAGTGGGG | | LLIYKASSLESGDPS | | | |
| | | ACCCATCAAGGTTCAGCGGCAGTGGATCTGGGACAGAATTCAC | | RFSGSGSGTEFTLTI | | | |
| | | TCTCACCATCAGCAGCCTGCAACCTGATGATTTTGCAACTTAT | | SSLQPDDFATYYCQQ | | | |
| | | TACTGCCAACAGTATAGTACTTACCCGTACACTTTTGGCCAGG | | YSTYPYTFGQGTKVE | | | |
| | | GGACCAAGGTGGAGATCAAA | | IK | | | |
| ADI-81790 | 2939 | AATTTTATGCTGACTCAGCCCCACTCTGTGTCGGAGTCTCCGG | 4921 | NFMLTQPHSVSESPG | 5274 | 2 | 2 |
| | | GGAAGACGGTAACCATCTCCTGCACCCGCAGCAGTGGCAGCAT | | KTVTISCTRSSGSIA | | | |
| | | TGCCAGCAGCTATGTGCAGTGGTACCAGCAGCGCCCGGGCAGT | | SSYVQWYQQRPGSSP | | | |
| | | TCCCCCACCACTGTGATCTATGACGATAACCAAAGACCCTCTG | | TTVIYDDNQRPSGVP | | | |
| | | GGGTCCCTGATCGGTTCTCTGGCTCCATCGACAGCTCCTCCAA | | DRFSGSIDSSSNSAS | | | |
| | | CTCTGCCTCCCTCACCATCTCTGGACTGAAGACTGAGGACGAG | | LTISGLKTEDEADYY | | | |
| | | GCTGACTACTACTGTCAGTCTCATGATTCTAGCACTGTGATAT | | CQSHDSSTVIFGGGT | | | |
| | | TCGGCGGAGGGACCAAGCTCACCGTCCTA | | KLTVL | | | |
| ADI-81755 | 2950 | GAAATGACACTCACGCAGTCTCCATCCTCCCTGTCTGCATCTG | 4922 | EMTLTQSPSSLSASV | 5275 | 2 | 2 |
| | | TAGGAGACAGGGTCACCATCACTTGCCAGGCGAGTCAGGCCAT | | GDRVTITCQASQAIS | | | |
| | | TAGCAACTATTTAAATTGGTATCAGCAGAAACCAGGGAAAGCC | | NYLNWYQQKPGKAPK | | | |
| | | CCTAAACTCCTGATCTACGATGCATCCAATTTGGAAACAGGGG | | LLIYDASNLETGVPS | | | |
| | | TCCCATCGAGGTTCAGTGGAAGTGGATCTGGGACAGATTTTAC | | RFSGSGSGTDFTFTI | | | |
| | | TTTCACCATCAGCAGCCTGCAGCCTGAAGATATTGCAACATAT | | SSLQPEDIATYFCQQ | | | |
| | | TTCTGTCAACAGTATGATAATCTCCCTTTCACTTTCGGCCCTG | | YDNLPFTFGPGTKVD | | | |
| | | GGACCAAGGTGGATATCAAA | | IK | | | |
| ADI-81893 | 2942 | GAAATTGGGGTGACGCAGTCTCCATCCTCCCTGTCTGCATCTG | 4923 | EIGVTQSPSSLSASV | 5276 | 2 | 2 |
| | | TAGGAGACAGAGTCACCATCACCTGCCGGGCAAGTCAGAGCAT | | GDRVTITCRASQSIS | | | |
| | | TAGCAGCTATTTAAATTGGTATCAGCAGAAGCCAGGGAAAGCC | | SYLNWYQQKPGKAPK | | | |
| | | CCTAAACTCCTGATTTATGCTGCATCCAGTTTGCAAAGTGGGG | | LLIYAASSLQSGVPS | | | |
| | | TCCCATCAAGGTTCAGTGGCAGTGGATTTGGGACAGATTTCAC | | RFSGSGFGTDFTFTI | | | |
| | | TTTCACCATCAGCAGTTTACAACCTGAGGATTTTGCAACTTAC | | SSLQPEDFATYYCQQ | | | |
| | | TATTGTCAACAGAGTTACAGAACCCCCCCCCTCACTTTCGGCC | | SYRTPPLTFGPGTKV | | | |
| | | CTGGGACCAAGGTGGAAATCAAA | | EIK | | | |
| ADI-81649 | 2942 | GATATTGTGCTGACGCAGTCTCCAGACTCCCTGGCTGTGTCTC | 4924 | DIVLTQSPDSLAVSL | 5277 | 2 | 2 |
| | | TGGGCGAGAGGGCCACCATCAACTGCAAGTCCAGCCAGAGTGT | | GERATINCKSSQSVL | | | |
| | | TTTATATAGTTCCAACAATGAGAACTACTTAGGTTGGTACCAG | | YSSNNENYLGWYQQK | | | |
| | | CAGAAACCAGGACAGCCTCCTAAGTTGCTCATTTACTGGGCAT | | PGQPPKLLIYWASTR | | | |
| | | CTACCCGGGAATCCGGGGTCCCTGACCGATTCAGTGGCAGCGG | | ESGVPDRFSGSGSGT | | | |
| | | GTCTGGGACAGATTTCACTCTCACCATCAGCAGCCTGCAGGCT | | DFTLTISSLQAEDVA | | | |
| | | GAAGATGTGGCAGTTTATTACTGTCAGCAATATTATAGTACTC | | VYYCQQYYSTPLTFG | | | |
| | | CCCTCACTTTCGGCCCTGGGACCAAGGTGGAGATCAAA | | PGTKVEIK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81716 | IGKV3-20 | EMALTQ | 3698 | RASQSV | 3885 | WYQQKP | 2258 | AASSRA | 2486 | GIPDRF | 2616 | HQYGYS | 4564 | FGQGTK |
| | | SPGTLS | | SSSFLA | | GQAPRL | | T | | SGSGSG | | PWT | | VDIK |
| | | LSPGER | | | | LIY | | | | TDFTLT | | | | |
| | | ATLSC | | | | | | | | ISRLEP | | | | |
| | | | | | | | | | | EDFAVY | | | | |
| | | | | | | | | | | YC | | | | |
| ADI-81910 | IGKV1-33 | DIGMTQ | 3564 | QASQDI | 3897 | WYQQKP | 4022 | DASNLE | 2416 | GVPSRF | 2537 | QQFDNL | 4565 | FGQGTR |
| | | SPSSLS | | SNSLN | | GKAPKR | | T | | SGSGSG | | PIT | | LEIK |
| | | ASVGDR | | | | LIY | | | | TDFTFT | | | | |
| | | VTITC | | | | | | | | ISSLQP | | | | |
| | | | | | | | | | | EDIATY | | | | |
| | | | | | | | | | | YC | | | | |
| ADI-81862 | IGLV3-21 | QSVLTQ | 2010 | GGNNIG | 2208 | WYQQKP | 2358 | DDSDRP | 2399 | GIPERF | 4321 | QVWDSN | 4566 | FGTGTK |
| | | PPSVSV | | SKSVH | | GQAPVL | | S | | SGSNSG | | SDHHYV | | LTVL |
| | | APGQTA | | | | VVN | | | | NTATLT | | | | |
| | | RITQ | | | | | | | | ISRVAA | | | | |
| | | | | | | | | | | GDEADY | | | | |
| | | | | | | | | | | YC | | | | |
| ADI-81898 | IGKV4-1 | DIRVTQ | 3699 | KSSQSV | 3898 | WYQQKP | 2290 | WASTRE | 2422 | GVPDRF | 2619 | QQYYST | 4567 | FGQGTK |
| | | SPDSLA | | LYSSNN | | GQPPKL | | S | | SGSGSG | | PWT | | VEIK |
| | | VSLGER | | KNFLA | | LIY | | | | TDFTLT | | | | |
| | | ATINC | | | | | | | | ISSLQA | | | | |
| | | | | | | | | | | EDVAVY | | | | |
| | | | | | | | | | | YC | | | | |
| ADI-81585 | IGKV3-11 | EIVLTQ | 1945 | RASQSV | 2119 | WYQQKP | 2258 | DASNRA | 2448 | GIPARF | 2607 | QQRDNW | 4568 | FGGGTK |
| | | SPATLS | | STYLA | | GQAPRL | | T | | SGSGSG | | VS | | VEIK |
| | | LSPGER | | | | LIY | | | | TDFTLT | | | | |
| | | ATLSC | | | | | | | | ISSLEP | | | | |
| | | | | | | | | | | EDFAVY | | | | |
| | | | | | | | | | | YC | | | | |
| ADI-81613 | IGKV4-1 | DIQMTQ | 1927 | KSSQSV | 3899 | WYQQKP | 2290 | WASTRE | 2422 | GVPDRF | 2619 | QQYYST | 4518 | FGGGTK |
| | | SPDSLA | | LYSSNN | | GQPPKL | | S | | SGSGSG | | PLT | | VEIK |
| | | VSLGER | | NNYLA | | LIY | | | | TDFTLT | | | | |
| | | ATING | | | | | | | | ISSLQA | | | | |
| | | | | | | | | | | EDVAVY | | | | |
| | | | | | | | | | | YC | | | | |
| ADI-81690 | IGKV3D-15 | EIVMTQ | 2034 | RASQSV | 2100 | WYQQKP | 2258 | GASTRA | 2383 | GIPARF | 2569 | QQYNKW | 4569 | FGQGTK |
| | | SPATLS | | SSSLA | | GQAPRL | | T | | SGSGSG | | PRYT | | VDIK |
| | | VSPGER | | | | LIY | | | | TEFTLT | | | | |
| | | ATLSC | | | | | | | | ISSLQS | | | | |
| | | | | | | | | | | EDFAVY | | | | |
| | | | | | | | | | | YC | | | | |
| ADI-81784 | IGKV1-33 | DIRMTQ | 1937 | QASQDI | 2212 | WYQQKP | 2257 | DASNLE | 2416 | GVPSRF | 4268 | QQYHSL | 4570 | FGPGTK |
| | | SPSSLS | | SNYLN | | GKAPKL | | T | | SGSGSG | | PIT | | VDIK |
| | | ASVGDR | | | | LIY | | | | TDFSFT | | | | |
| | | VTITQ | | | | | | | | ISSLQP | | | | |
| | | | | | | | | | | EDIATY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81716 | 2944 | GAAATGGCACTGACGCAGTCTCCAGGCACCCTGTCTTTGTCTC | 4925 | EMALTQSPGTLSLSP | 5278 | 2 | 2 |
| | | CAGGGGAAAGAGCCACCCTCTCCTGCAGGGCCAGTCAGAGTGT | | GERATLSCRASQSVS | | | |
| | | TAGCAGCAGCTTCTTAGCATGGTACCAGCAGAAACCTGGCCAG | | SSFLAWYQQKPGQAP | | | |
| | | GCTCCCAGGCTCCTCATCTATGCTGCATCCAGCAGGGCCACTG | | RLLIYAASSRATGIP | | | |
| | | GCATCCCAGACAGGTTCAGTGGCAGTGGGTCTGGGACAGACTT | | DRFSGSGSGTDFTLT | | | |
| | | CACTCTCACCATCAGCAGACTGGAGCCTGAAGATTTTGCAGTG | | ISRLEPEDFAVYYCH | | | |
| | | TATTACTGTCACCAGTATGGTTACTCACCGTGGACGTTCGGCC | | QYGYSPWTFGQGTKV | | | |
| | | AAGGGACCAAAGTGGATATCAAA | | DIK | | | |
| ADI-81910 | 2940 | GACATCGGGATGACCCAGTCTCCATCCTCCCTGTCTGCATCTG | 4926 | DIGMTQSPSSLSASV | 5279 | 2 | 2 |
| | | TAGGAGACAGAGTCACCATCACTTGCCAGGCGAGTCAGGACAT | | GDRVTITCQASQDIS | | | |
| | | TAGCAACTCTTTAAATTGGTATCAGCAGAAACCAGGGAAAGCC | | NSLNWYQQKPGKAPK | | | |
| | | CCTAAGCGCCTGATCTACGATGCATCCAATTTGGAAACAGGGG | | RLIYDASNLETGVPS | | | |
| | | TCCCATCAAGGTTCAGTGGAAGTGGATCTGGGACAGATTTTAC | | RFSGSGSGTDFTFTI | | | |
| | | TTTCACCATCAGCAGCCTGCAGCCTGAAGATATTGCAACATAT | | SSLQPEDIATYYCQQ | | | |
| | | TACTGTCAACAGTTTGATAATCTCCCGATCACCTTCGGCCAAG | | FDNLPITFGQGTRLE | | | |
| | | GGACACGACTGGAGATTAAA | | IK | | | |
| ADI-81862 | 2949 | CAGTCTGTGCTGACTCAGCCACCCTCGGTGTCAGTGGCCCCAG | 4927 | QSVLTQPPSVSVAPG | 5280 | 2 | 2 |
| | | GACAGACGGCCAGGATTACCTGTGGGGGAAACAACATTGGAAG | | QTARITCGGNNIGSK | | | |
| | | TAAAAGTGTGCACTGGTACCAGCAGAAGCCAGGCCAGGCCCCT | | SVHWYQQKPGQAPVL | | | |
| | | GTGCTGGTCGTCAATGATGATAGCGACCGGCCCTCAGGGATCC | | VVNDDSDRPSGIPER | | | |
| | | CTGAGCGATTCTCTGGCTCCAACTCTGGGAACACGGCCACCCT | | FSGSNSGNTATLTIS | | | |
| | | GACCATCAGCAGGGTCGCAGCCGGGGATGAGGCCGACTATTAC | | RVAAGDEADYYCQVW | | | |
| | | TGTCAGGTGTGGGATAGTAATAGTGATCATCATTATGTCTTCG | | DSNSDHHYVFGTGTK | | | |
| | | GAACTGGGACCAAGCTCACCGTCCTA | | LTVL | | | |
| ADI-81898 | 2937 | GACATCCGAGTGACGCAGTCTCCAGACTCCCTGGCTGTGTCTC | 4928 | DIRVTQSPDSLAVSL | 5281 | 1 | 1 |
| | | TGGGCGAGAGGGCCACCATCAACTGCAAGTCCAGCCAGAGTGT | | GERATINCKSSQSVL | | | |
| | | TTTATACAGCTCCAACAATAAGAACTTCTTAGCTTGGTACCAG | | YSSNNKNFLAWYQQK | | | |
| | | CAGAAACCAGGACAGCCTCCTAAGCTGCTCATTTATTGGGCAT | | PGQPPKLLIYWASTR | | | |
| | | CTACCCGGGAATCCGGGGTCCCTGACCGATTCAGTGGCAGCGG | | ESGVPDRFSGSGSGT | | | |
| | | GTCTGGGACAGATTTCACTCTCACCATCAGCAGCCTGCAGGCT | | DFTLTISSLQAEDVA | | | |
| | | GAAGATGTGGCAGTTTATTACTGTCAGCAATATTACAGTACTC | | VYYCQQYYSTPWTFG | | | |
| | | CGTGGACGTTCGGCCAAGGGACCAAGGTGGAAATCAAA | | QGTKVEIK | | | |
| ADI-81585 | 2936 | GAAATTGTGTTGACGCAGTCTCCAGCCACCCTGTCTTTGTCTC | 4929 | EIVLTQSPATLSLSP | 5282 | 1 | 1 |
| | | CAGGGGAAAGAGCCACCCTGTCCTGCAGGGCCAGTCAGAGTGT | | GERATLSCRASQSVS | | | |
| | | TAGCACCTACTTAGCCTGGTACCAACAGAAACCTGGCCAGGCT | | TYLAWYQQKPGQAPR | | | |
| | | CCCAGGCTCCTCATCTATGATGCATCCAACAGGGCCACTGGCA | | LLIYDASNRATGIPA | | | |
| | | TCCCGGCCAGGTTCAGTGGCAGTGGGTCTGGGACAGACTTCAC | | RFSGSGSGTDFTLTI | | | |
| | | TCTCACCATCAGCAGCCTAGAGCCTGAAGATTTTGCAGTTTAT | | SSLEPEDFAVYYCQQ | | | |
| | | TACTGTCAGCAGCGTGACAACTGGGTCTCTTTCGGCGGAGGGA | | RDNWVSFGGGTKVEI | | | |
| | | CCAAGGTGGAAATCAAA | | K | | | |
| ADI-81613 | 2936 | GACATCCAGATGACCCAGTCTCCAGACTCCCTGGCTGTGTCTC | 4930 | DIQMTQSPDSLAVSL | 5283 | 1 | 1 |
| | | TGGGCGAGAGGGCCACCATCAACTGCAAGTCCAGCCAGAGTGT | | GERATINCKSSQSVL | | | |
| | | TTTATACAGCTCCAACAATAACAACTACTTAGCTTGGTACCAG | | YSSNNNNYLAWYQQK | | | |
| | | CAGAAACCAGGACAGCCTCCTAAGCTGCTCATTTACTGGGCAT | | PGQPPKLLIYWASTR | | | |
| | | CTACCCGGGAATCCGGGGTCCCTGACCGATTCAGTGGCAGCGG | | ESGVPDRFSGSGSGT | | | |
| | | GTCTGGGACCGATTTCACTCTCACCATCAGCAGCCTGCAGGCT | | DFTLTISSLQAEDVA | | | |
| | | GAGGATGTTGCAGTTTATTACTGTCAGCAATATTATAGTACTC | | VYYCQQYYSTPLTFG | | | |
| | | CGCTCACTTTCGGCGGAGGGACCAAGGTGGAAATCAAA | | GGTKVEIK | | | |
| ADI-81690 | 2944 | GAAATTGTGATGACTCAGTCTCCAGCCACCCTGTCTGTGTCTC | 4931 | EIVMTQSPATLSVSP | 5284 | 1 | 1 |
| | | CAGGGGAAAGAGCCACCCTCTCCTGCAGGGCCAGTCAGAGTGT | | GERATLSCRASQSVS | | | |
| | | TAGCAGCAGCTTAGCCTGGTACCAGCAGAAACCTGGCCAGGCT | | SSLAWYQQKPGQAPR | | | |
| | | CCCAGGCTCCTCATCTATGGTGCATCCACCAGGGCCACTGGTA | | LLIYGASTRATGIPA | | | |
| | | TCCCAGCCAGGTTCAGTGGCAGTGGGTCTGGGACAGAGTTCAC | | RFSGSGSGTEFTLTI | | | |
| | | TCTCACCATCAGTAGCCTGCAGTCTGAAGATTTTGCAGTTTAT | | SSLQSEDFAVYYCQQ | | | |
| | | TACTGTCAGCAGTATAATAAGTGGCCTCGGTACACTTTTGGCC | | YNKWPRYTFGQGTKV | | | |
| | | AGGGGACCAAGGTGGATATCAAA | | DIK | | | |
| ADI-81784 | 2950 | GACATCCGGATGACCCAGTCTCCATCTTCCCTGTCTGCATCTG | 4932 | DIRMTQSPSSLSASV | 5285 | 1 | 1 |
| | | TAGGAGACAGAGTCACCATCACCTGCCAGGCGAGTCAGGACAT | | GDRVTITCQASQDIS | | | |
| | | AAGCAACTATTTAAATTGGTATCAGCAAAAACCAGGGAAAGCC | | NYLNWYQQKPGKAPK | | | |
| | | CCTAAGCTCCTGATCTACGATGCATCCAATTTGGAAACAGGGG | | LLIYDASNLETGVPS | | | |
| | | TCCCATCACGGTTCAGTGGAAGTGGATCTGGGACAGATTTTAG | | RFSGSGSGTDFSFTI | | | |
| | | TTTCACCATCAGCAGCCTGCAGCCTGAAGATATTGCAACATAT | | SSLQPEDIATYYCQQ | | | |
| | | TACTGTCAACAGTATCATAGTCTCCCGATCACTTTCGGCCCTG | | YHSLPITFGPGTKVD | | | |
| | | GGACCAAGGTGGATATCAAA | | IK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81863 | IGKV3-11 | EMTLTQ | 3534 | RASQSV | 3900 | WYQQKP | 2258 | DASNRA | 2448 | GIPARF | 2607 | QQRSNW | 4571 | FGGGTK |
| | | SPATLS | | RSYLA | | GQAPRL | | T | | SGSGSG | | PRTLT | | VEIK |
| | | LSPGER | | | | LIY | | | | TDFTLT | | | | |
| | | ATLSC | | | | | | | | ISSLEP | | | | |
| | | | | | | | | | | EDFAVY | | | | |
| | | | | | | | | | | YC | | | | |
| ADI-81645 | IGKV3-11 | EIVLTQ | 3581 | RASQSV | 2168 | WYQQKP | 2258 | DASNRA | 2448 | GIPARF | 2607 | QQRGNW | 4572 | FGGGTK |
| | | SPGTLS | | SSYLA | | GQAPRL | | T | | SGSGSG | | PGT | | VEIK |
| | | LSPGER | | | | LIY | | | | TDFTLT | | | | |
| | | ATLSC | | | | | | | | ISSLEP | | | | |
| | | | | | | | | | | EDFAVY | | | | |
| | | | | | | | | | | YC | | | | |
| ADI-81806 | IGKV1D-39 | DIPMTQ | 1947 | RASQRI | 2135 | WYQQKP | 2257 | AASSLQ | 2391 | GVPSRF | 2534 | QQSYST | 2807 | FGGGTK |
| | | SPSSLS | | SSYLN | | GKAPKL | | S | | SGSGSG | | PPVT | | VEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81801 | IGKV1D-39 | EIPMTQ | 1912 | RASQRI | 2135 | WYQQKP | 2257 | AASSLQ | 2391 | GVPSRF | 2534 | QQSYST | 2807 | FGGGTK |
| | | SPSSLS | | SSYLN | | GKAPKL | | S | | SGSGSG | | PPVT | | VDIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81769 | IGKV1-12 | DIEMTQ | 3568 | RASQGI | 3901 | WYQQKP | 2257 | AASSLQ | 2391 | GVPSRF | 2534 | QQTISF | 4573 | FGQGTR |
| | | SPSSVS | | SNWLA | | GKAPKL | | S | | SGSGSG | | PAIT | | LEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81625 | IGKV1-33 | DIQMTQ | 1896 | QASQDI | 2212 | WYQQKP | 2257 | DASNLE | 2416 | GVPSRF | 2537 | QQHDNV | 4574 | FGQGTR |
| | | SPSSLS | | SNYLN | | GKAPKL | | T | | SGSGSG | | PLT | | LEIK |
| | | ASVGDR | | | | LIY | | | | TDFTFT | | | | |
| | | VTIAC | | | | | | | | ISSLQP | | | | |
| | | | | | | | | | | EDIATY | | | | |
| | | | | | | | | | | YC | | | | |
| ADI-81741 | IGKV1-9 | DIPMTQ | 3700 | RASQGI | 2136 | WYQQKP | 2257 | TASTLQ | 4151 | GVPSRF | 2592 | QQLNNH | 4575 | FGQGTR |
| | | SPSFLS | | SSYLA | | GKAPKL | | S | | SGSGSG | | PPA | | LEIK |
| | | ASVGDR | | | | LIY | | | | TEFTLT | | | | |
| | | VTITC | | | | | | | | ISSLQP | | | | |
| | | | | | | | | | | EDFATY | | | | |
| | | | | | | | | | | YC | | | | |
| ADI-81761 | IGKV3-11 | EIPLTQ | 3701 | RASQSV | 3871 | WYQQKP | 2258 | DASNRA | 2448 | GIPARF | 2607 | QQRSKW | 4576 | FGQGTK |
| | | SPATLS | | SNYLA | | GQAPRL | | T | | SGSGSG | | RYT | | VEIK |
| | | LSPGER | | | | LIY | | | | TDFTLT | | | | |
| | | ATLSC | | | | | | | | ISSLEP | | | | |
| | | | | | | | | | | EDFAVY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81863 | 2936 | GAAATGACACTGACGCAGTCTCCAGCCACCCTGTCTTTGTCTC | 4933 | EMTLTQSPATLSLSP | 5286 | 1 | 1 |
| | | CAGGGGAAAGAGCCACTCTCTCCTGCAGGGCCAGTCAGAGCGT | | GERATLSCRASQSVR | | | |
| | | TCGCAGCTACTTAGCCTGGTACCAACAGAAACCTGGCCAGGCT | | SYLAWYQQKPGQAPR | | | |
| | | CCCAGGCTCCTCATCTATGATGCCTCCAACAGGGCCACTGGCA | | LLIYDASNRATGIPA | | | |
| | | TCCCAGCCAGGTTCAGTGGCAGTGGGTCTGGGACAGACTTCAC | | RFSGSGSGTDFTLTI | | | |
| | | TCTCACCATCAGCAGCCTAGAGCCTGAAGATTTTGCAGTTTAT | | SSLEPEDFAVYYCQQ | | | |
| | | TACTGTCAGCAGCGTAGCAACTGGCCCCGCACACTCACTTTCG | | RSNWPRTLTFGGGTK | | | |
| | | GCGGAGGGACCAAGGTGGAAATCAAA | | VEIK | | | |
| ADI-81645 | 2936 | GAAATTGTGCTGACGCAGTCTCCAGGCACCCTGTCTTTGTCTC | 4934 | EIVLTQSPGTLSLSP | 5287 | 1 | 1 |
| | | CAGGGGAAAGAGCCACCCTCTCCTGCAGGGCCAGTCAGAGTGT | | GERATLSCRASQSVS | | | |
| | | TAGCAGCTACTTAGCCTGGTACCAACAAAAACCTGGCCAGGCT | | SYLAWYQQKPGQAPR | | | |
| | | CCCAGGCTCCTCATCTATGATGCGTCCAACAGGGCCACTGGCA | | LLIYDASNRATGIPA | | | |
| | | TCCCAGCCAGGTTCAGTGGCAGTGGGTCTGGGACAGACTTCAC | | RFSGSGSGTDFTLTI | | | |
| | | TCTCACCATCAGCAGCCTGGAGCCTGAAGATTTTGCAGTTTAT | | SSLEPEDFAVYYCQQ | | | |
| | | TACTGTCAGCAGCGTGGCAACTGGCCGGGGACTTTCGGCGGAG | | RGNWPGTFGGGTKVE | | | |
| | | GGACCAAGGTGGAAATCAAA | | IK | | | |
| ADI-81806 | 2936 | GACATCCCGATGACCCAGTCTCCATCCTCCCTGTCTGCATCTG | 4935 | DIPMTQSPSSLSASV | 5288 | 1 | 1 |
| | | TAGGAGACAGAGTCACCATCACTTGCCGGGCAAGTCAGAGAAT | | GDRVTITCRASQRIS | | | |
| | | AAGCAGCTATTTAAATTGGTATCAGCAGAAACCAGGGAAAGCC | | SYLNWYQQKPGKAPK | | | |
| | | CCTAAGCTCCTGATCTATGCTGCATCCAGTTTGCAAAGTGGGG | | LLIYAASSLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAACCTGAAGATTTTGCAACTTAC | | SSLQPEDFATYYCQQ | | | |
| | | TACTGTCAACAGAGTTACAGTACCCCTCCGGTCACTTTCGGCG | | SYSTPPVTFGGGTKV | | | |
| | | GAGGGACCAAGGTGGAAATCAAA | | EIK | | | |
| ADI-81801 | 2938 | GAAATCCCGATGACCCAGTCTCCATCCTCCCTGTCTGCATCTG | 4936 | EIPMTQSPSSLSASV | 5289 | 1 | 1 |
| | | TAGGAGACAGAGTCACCATCACTTGCCGGGCAAGTCAGAGAAT | | GDRVTITCRASQRIS | | | |
| | | AAGCAGCTATTTAAATTGGTATCAGCAGAAACCAGGGAAAGCC | | SYLNWYQQKPGKAPK | | | |
| | | CCTAAGCTCCTGATCTATGCTGCATCCAGTTTGCAAAGTGGGG | | LLIYAASSLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAACCTGAAGATTTTGCAACTTAC | | SSLQPEDFATYYCQQ | | | |
| | | TACTGTCAACAGAGTTACAGTACCCCTCCGGTCACTTTCGGCG | | SYSTPPVTFGGGTKV | | | |
| | | GAGGGACCAAGGTGGATATCAAA | | DIK | | | |
| ADI-81769 | 2940 | GACATCGAGATGACCCAGTCTCCATCTTCCGTGTCTGCATCTG | 4937 | DIEMTQSPSSVSASV | 5290 | 1 | 1 |
| | | TGGGAGACAGAGTCACCATCACTTGTCGGGCGAGTCAGGGTAT | | GDRVTITCRASQGIS | | | |
| | | TAGCAACTGGTTAGCCTGGTATCAGCAGAAACCAGGGAAAGCC | | NWLAWYQQKPGKAPK | | | |
| | | CCTAAACTCCTGATCTATGCTGCATCCAGTTTGCAAAGTGGGG | | LLIYAASSLQSGVPS | | | |
| | | TCCCATCAAGGTTCAGCGGCAGTGGATCTGGGACAGATTTCAC | | RFSGSGSGTDFTLTI | | | |
| | | TCTCACCATCAGCAGCCTGCAGCCTGAAGATTTTGCAACTTAC | | SSLQPEDFATYYCQQ | | | |
| | | TATTGTCAACAGACTATCAGTTTTCCGGCGATCACCTTCGGCC | | TISFPAITFGQGTRL | | | |
| | | AAGGGACACGACTGGAGATTAAA | | EIK | | | |
| ADI-81625 | 2940 | GACATCCAGATGACCCAGTCTCCATCCTCCCTGTCTGCATCTG | 4938 | DIQMTQSPSSLSASV | 5291 | 1 | 1 |
| | | TAGGAGACAGAGTCACCATCGCTTGCCAGGCGAGTCAGGACAT | | GDRVTIACQASQDIS | | | |
| | | TAGCAACTATTTAAATTGGTATCAGCAAAAACCAGGGAAAGCC | | NYLNWYQQKPGKAPK | | | |
| | | CCTAAGCTCCTGATCTACGATGCATCCAATTTGGAAACAGGGG | | LLIYDASNLETGVPS | | | |
| | | TCCCATCAAGGTTCAGTGGAAGTGGATCTGGGACAGATTTTAC | | RFSGSGSGTDFTFTI | | | |
| | | TTTCACCATCAGCAGCCTGCAGCCTGAAGATATTGCAACATAT | | SSLQPEDIATYYCQQ | | | |
| | | TACTGTCAACAGCATGATAATGTCCCTCTCACCTTCGGCCAAG | | HDNVPLTFGQGTRLE | | | |
| | | GGACACGACTGGAGATTAAA | | IK | | | |
| ADI-81741 | 2940 | GACATCCCAATGACCCAGTCTCCATCCTTCCTGTCTGCATCTG | 4939 | DIPMTQSPSFLSASV | 5292 | 1 | 1 |
| | | TAGGAGACAGAGTCACCATCACTTGCCGGGCCAGTCAGGGCAT | | GDRVTITCRASQGIS | | | |
| | | TAGCAGTTATTTAGCCTGGTATCAGCAAAAACCAGGGAAAGCC | | SYLAWYQQKPGKAPK | | | |
| | | CCTAAGCTCCTGATCTATACTGCATCCACTTTGCAAAGTGGGG | | LLIYTASTLQSGVPS | | | |
| | | TCCCATCAAGGTTCAGCGGCAGTGGATCTGGGACAGAATTCAC | | RFSGSGSGTEFTLTI | | | |
| | | TCTCACAATCAGCAGCCTGCAGCCTGAAGATTTTGCAACTTAT | | SSLQPEDFATYYCQQ | | | |
| | | TACTGTCAACAGCTTAATAATCACCCTCCGGCCTTCGGCCAAG | | LNNHPPAFGQGTRLE | | | |
| | | GGACACGACTGGAGATTAAA | | IK | | | |
| ADI-81761 | 2937 | GAAATCCCACTGACCCAGTCTCCAGCCACCCTGTCTTTGTCTC | 4940 | EIPLTQSPATLSLSP | 5293 | 1 | 1 |
| | | CAGGGGAAAGAGCCACCCTCTCCTGCAGGGCCAGTCAGAGTGT | | GERATLSCRASQSVS | | | |
| | | TAGCAACTACTTAGCCTGGTACCAACAGAAACCTGGCCAGGCT | | NYLAWYQQKPGQAPR | | | |
| | | CCCAGGCTCCTCATCTATGATGCATCCAACAGGGCCACTGGCA | | LLIYDASNRATGIPA | | | |
| | | TCCCAGCCAGGTTCAGTGGCAGTGGGTCTGGGACAGACTTCAC | | RFSGSGSGTDFTLTI | | | |
| | | TCTCACCATCAGCAGCCTAGAGCCTGAAGATTTTGCAGTTTAT | | SSLEPEDFAVYYCQQ | | | |
| | | TACTGTCAGCAGCGTAGCAAGTGGCGGTACACTTTTGGCCAGG | | RSKWRYTFGQGTKVE | | | |
| | | GGACCAAGGTGGAAATCAAA | | IK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81867 | IGKV2D-28 | DIRMTQ | 3702 | RSSQSL | 3877 | WYLQKP | 2260 | LGSNRA | 2387 | GVPDRF | 2533 | MQALQS | 4577 | FGQGTR |
| | | SPLSLP | | LHSNGH | | GQSPQL | | S | | SGSGSG | | PPA | | LEIK |
| | | VTPGEP | | NYLD | | LIY | | | | TDFTLK | | | | |
| | | ASISC | | | | | | | | ISRVEA | | | | |
| | | | | | | | | | | EDVGVY | | | | |
| | | | | | | | | | | YC | | | | |
| ADI-81655 | IGLV6-57 | NLMLTQ | 1922 | TGSSGS | 3902 | WYQQRP | 2298 | EDDQRP | 2502 | GVPDRF | 2574 | QSYDSS | 4578 | FGGGTK |
| | | PHSVSE | | IASNYV | | GSAPTT | | S | | SGSIDS | | RVV | | LTVL |
| | | SPGKTV | | Q | | VIY | | | | SSNSAS | | | | |
| | | TISC | | | | | | | | LTISGL | | | | |
| | | | | | | | | | | KTEDEA | | | | |
| | | | | | | | | | | DYYC | | | | |
| ADI-81791 | IGKV3D-15 | EIVLTQ | 2033 | RASQSV | 2227 | WYQQKP | 2258 | GASTRA | 2383 | GIPARF | 4322 | QQYNNW | 4559 | FGGGTK |
| | | SPATLS | | SSNLA | | GQAPRL | | T | | SGSGSG | | PPLT | | VDIK |
| | | VSPGER | | | | LIY | | | | TEFTLT | | | | |
| | | ATLSC | | | | | | | | ISSLQS | | | | |
| | | | | | | | | | | EDFALY | | | | |
| | | | | | | | | | | YC | | | | |
| ADI-81842 | IGKV3-11 | EIVLTQ | 1945 | RASQSV | 2168 | WYQQKP | 2258 | DASNRA | 2448 | GIPARF | 4323 | QQRSIW | 4579 | FGPGTK |
| | | SPATLS | | SSYLA | | GQAPRL | | T | | SGSGSG | | PPPFT | | VEIK |
| | | LSPGER | | | | LIY | | | | TDFNLT | | | | |
| | | ATLSC | | | | | | | | ISSLEP | | | | |
| | | | | | | | | | | EDFAVY | | | | |
| | | | | | | | | | | YC | | | | |
| ADI-81846 | IGKV3-11 | EIVLTQ | 1945 | RASQSV | 2168 | WYQQKP | 3906 | DASNRA | 2448 | GIPARF | 2607 | QQRSNW | 4580 | FGQGTK |
| | | SPATLS | | SSYLA | | GQAPRV | | T | | SGSGSG | | RYT | | VEIK |
| | | LSPGER | | | | LIY | | | | TDFTLT | | | | |
| | | ATLSC | | | | | | | | ISSLEP | | | | |
| | | | | | | | | | | EDFAVY | | | | |
| | | | | | | | | | | YC | | | | |
| ADI-81894 | IGKV1D-39 | EIPMTQ | 1912 | RASQSI | 2050 | WYQQKP | 2257 | AASGLQ | 4152 | GVPSRF | 2534 | QQSYTN | 4581 | FGQGTK |
| | | SPSSLS | | SSYLN | | GKAPKL | | S | | SGSGSG | | PMYT | | VEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81912 | IGKV2D-28 | EMTLTQ | 3703 | RSSQSL | 3887 | WYLQKP | 2260 | LGSNRA | 2387 | GVPDRF | 4324 | MQALQT | 4582 | FGQGTK |
| | | SPLSLP | | LHSNGY | | GQSPQL | | S | | SGSGSG | | PPYG | | VDIK |
| | | VTPGEP | | NYLD | | LIY | | | | TDFTLK | | | | |
| | | ASISC | | | | | | | | ISRVEA | | | | |
| | | | | | | | | | | GDVGVY | | | | |
| | | | | | | | | | | YC | | | | |
| ADI-81848 | IGLV2-23 | QSVLTQ | 1918 | TGTSSD | 3903 | WYQQHP | 2265 | EVTKRP | 2449 | GVSNRF | 4325 | CSYAGI | 4583 | FGTGTK |
| | | PASVSG | | VGSYNL | | GKAPKL | | S | | SGSKSG | | YV | | LTVL |
| | | SPGQSI | | VS | | MIY | | | | NTASLT | | | | |
| | | TISC | | | | | | | | ISGLQA | | | | |
| | | | | | | | | | | EDEADY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81867 | 2940 | GACATCCGGATGACCCAGTCTCCACTCTCCCTGCCCGTCACCC | 4941 | DIRMTQSPLSLPVTP | 5294 | 1 | 1 |
| | | CTGGAGAGCCGGCCTCCATCTCCTGCAGGTCTAGTCAGAGCCT | | GEPASISCRSSQSLL | | | |
| | | CCTGCATAGTAATGGACACAACTATTTGGATTGGTACCTGCAG | | HSNGHNYLDWYLQKP | | | |
| | | AAGCCAGGGCAGTCTCCACAGCTCCTGATCTATTTGGGTTCTA | | GQSPQLLIYLGSNRA | | | |
| | | ATCGGGCCTCCGGGGTCCCTGACAGGTTCAGTGGCAGTGGATC | | SGVPDRFSGSGSGTD | | | |
| | | AGGCACAGATTTTACACTGAAAATCAGCAGAGTGGAGGCTGAG | | FTLKISRVEAEDVGV | | | |
| | | GATGTTGGGGTTTATTACTGCATGCAAGCTCTACAAAGTCCTC | | YYCMQALQSPPAFGQ | | | |
| | | CGGCCTTCGGCCAAGGGACACGACTGGAGATTAAA | | GTRLEIK | | | |
| ADI-81655 | 2939 | AATCTTATGCTGACTCAGCCCCACTCTGTGTCGGAGTCTCCGG | 4942 | NLMLTQPHSVSESPG | 5295 | 1 | 1 |
| | | GGAAGACGGTAACCATCTCCTGCACCGGCAGCAGTGGCAGCAT | | KTVTISCTGSSGSIA | | | |
| | | TGCCAGCAACTATGTGCAGTGGTACCAGCAGCGCCCGGGCAGT | | SNYVQWYQQRPGSAP | | | |
| | | GCCCCCACCACTGTGATCTATGAGGATGACCAAAGACCCTCTG | | TTVIYEDDQRPSGVP | | | |
| | | GGGTCCCTGATCGGTTCTCTGGCTCCATCGACAGCTCCTCCAA | | DRFSGSIDSSSNSAS | | | |
| | | CTCTGCCTCCCTCACCATCTCTGGACTGAAGACTGAGGACGAG | | LTISGLKTEDEADYY | | | |
| | | GCTGACTACTACTGTCAGTCTTATGATAGCAGCCGGGTGGTAT | | CQSYDSSRVVFGGGT | | | |
| | | TCGGCGGAGGGACCAAGCTCACCGTCCTA | | KLTVL | | | |
| ADI-81791 | 2938 | GAAATTGTATTGACGCAGTCTCCAGCCACCCTGTCTGTGTCTC | 4943 | EIVLTQSPATLSVSP | 5296 | 1 | 1 |
| | | CAGGGGAAAGAGCCACCCTCTCCTGCAGGGCCAGTCAGAGTGT | | GERATLSCRASQSVS | | | |
| | | TAGCAGCAACTTAGCCTGGTACCAGCAGAAACCTGGCCAGGCT | | SNLAWYQQKPGQAPR | | | |
| | | CCCAGGCTCCTCATCTATGGTGCATCCACCAGGGCCACTGGTA | | LLIYGASTRATGIPA | | | |
| | | TCCCAGCCAGGTTCAGTGGCAGTGGGTCTGGGACAGAGTTCAC | | RFSGSGSGTEFTLTI | | | |
| | | TCTCACCATCAGCAGCCTGCAGTCTGAAGATTTTGCACTTTAT | | SSLQSEDFALYYCQQ | | | |
| | | TACTGTCAGCAGTATAATAACTGGCCTCCGCTCACTTTCGGCG | | YNNWPPLTFGGGTKV | | | |
| | | GAGGGACCAAAGTGGATATCAAA | | DIK | | | |
| ADI-81842 | 2942 | GAAATTGTGCTGACACAGTCTCCAGCCACCCTGTCTTTGTCTC | 4944 | EIVLTQSPATLSLSP | 5297 | 1 | 1 |
| | | CAGGGGAAAGAGCCACCCTCTCCTGCAGGGCCAGTCAGAGTGT | | GERATLSCRASQSVS | | | |
| | | TAGCAGCTACTTAGCCTGGTACCAACAGAAACCTGGCCAGGCT | | SYLAWYQQKPGQAPR | | | |
| | | CCCAGGCTCCTCATCTATGATGCATCCAACAGGGCCACTGGCA | | LLIYDASNRATGIPA | | | |
| | | TCCCAGCCAGGTTCAGTGGCAGTGGGTCTGGGACAGACTTCAA | | RFSGSGSGTDFNLTI | | | |
| | | TCTCACCATCAGCAGCCTAGAGCCTGAGGATTTTGCAGTTTAT | | SSLEPEDFAVYYCQQ | | | |
| | | TACTGTCAGCAGCGTAGCATCTGGCCTCCGCCATTCACTTTCG | | RSIWPPPFTFGPGTK | | | |
| | | GCCCTGGGACCAAGGTGGAAATCAAA | | VEIK | | | |
| ADI-81846 | 2937 | GAAATTGTGTTGACGCAGTCTCCAGCCACCCTGTCTTTGTCTC | 4945 | EIVLTQSPATLSLSP | 5298 | 1 | 1 |
| | | CAGGGGAAAGAGCCACCCTCTCCTGCAGGGCCAGTCAGAGTGT | | GERATLSCRASQSVS | | | |
| | | TAGCAGCTACTTAGCCTGGTACCAACAGAAACCTGGCCAGGCT | | SYLAWYQQKPGQAPR | | | |
| | | CCCAGGGTCCTCATCTATGATGCATCCAACAGGGCCACTGGCA | | VLIYDASNRATGIPA | | | |
| | | TCCCAGCCAGGTTCAGTGGCAGTGGGTCTGGGACAGACTTCAC | | RFSGSGSGTDFTLTI | | | |
| | | TCTCACCATCAGCAGCCTAGAGCCTGAAGATTTTGCAGTTTAT | | SSLEPEDFAVYYCQQ | | | |
| | | TACTGTCAGCAGCGTAGCAACTGGCGGTACACTTTTGGCCAGG | | RSNWRYTFGQGTKVE | | | |
| | | GGACCAAGGTGGAAATCAAA | | IK | | | |
| ADI-81894 | 2937 | GAAATCCCGATGACCCAGTCTCCATCCTCCCTGTCTGCATCTG | 4946 | EIPMTQSPSSLSASV | 5299 | 1 | 1 |
| | | TAGGAGACAGAGTCACCATCACTTGCCGGGCAAGTCAGAGCAT | | GDRVTITCRASQSIS | | | |
| | | TAGCAGCTATTTAAATTGGTATCAGCAGAAACCAGGGAAAGCC | | SYLNWYQQKPGKAPK | | | |
| | | CCTAAGCTCCTGATCTATGCTGCATCCGGTTTGCAAAGTGGGG | | LLIYAASGLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAACCTGAAGATTTTGCAACTTAC | | SSLQPEDFATYYCQQ | | | |
| | | TACTGTCAACAGAGTTACACTAACCCCATGTACACTTTTGGCC | | SYTNPMYTFGQGTKV | | | |
| | | AGGGGACCAAGGTGGAAATCAAA | | EIK | | | |
| ADI-81912 | 2944 | GAAATGACACTGACGCAGTCTCCACTCTCCCTGCCCGTCACCC | 4947 | EMTLTQSPLSLPVTP | 5300 | 1 | 1 |
| | | CTGGAGAGCCGGCCTCCATCTCCTGCAGGTCTAGTCAGAGCCT | | GEPASISCRSSQSLL | | | |
| | | CCTGCATAGTAATGGATACAACTATTTGGATTGGTACCTGCAG | | HSNGYNYLDWYLQKP | | | |
| | | AAGCCAGGGCAGTCTCCACAGCTCCTGATCTATTTGGGTTCTA | | GQSPQLLIYLGSNRA | | | |
| | | ATCGGGCCTCCGGGGTCCCTGACAGGTTCAGTGGCAGTGGATC | | SGVPDRFSGSGSGTD | | | |
| | | AGGCACAGATTTTACACTGAAAATCAGCAGAGTGGAGGCTGGG | | FTLKISRVEAGDVGV | | | |
| | | GATGTTGGGGTTTATTACTGCATGCAAGCTCTACAAACTCCTC | | YYCMQALQTPPYGFG | | | |
| | | CGTACGGTTTTGGCCAGGGGACCAAGGTGGATATCAAA | | QGTKVDIK | | | |
| ADI-81848 | 2949 | CAGTCTGTCCTGACTCAGCCTGCCTCCGTGTCTGGGTCTCCTG | 4948 | QSVLTQPASVSGSPG | 5301 | 1 | 1 |
| | | GACAGTCGATCACCATCTCCTGCACTGGAACCAGCAGTGATGT | | QSITISCTGTSSDVG | | | |
| | | TGGGAGTTATAACCTTGTCTCCTGGTACCAACAGCACCCAGGC | | SYNLVSWYQQHPGKA | | | |
| | | AAAGCCCCCAAACTCATGATTTATGAGGTCACTAAGCGGCCCT | | PKLMIYEVTKRPSGV | | | |
| | | CAGGGGTTTCTAATCGCTTCTCTGGCTCCAAGTCTGGCAACAC | | SNRFSGSKSGNTASL | | | |
| | | GGCCTCCCTGACAATCTCTGGGCTCCAGGCTGAGGACGAGGCT | | TISGLQAEDEADYYC | | | |
| | | GATTATTACTGCTGCTCATATGCAGGTATTTATGTCTTCGGAA | | CSYAGIYVFGTGTKL | | | |
| | | CTGGGACCAAGCTCACCGTCCTA | | TVL | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81915 | IGKV4-1 | EIVMTQ | 3704 | KSSQSV | 3904 | WYQQKP | 2290 | WASTRE | 2422 | GVPDRF | 2619 | QQYYST | 4584 | FGGGTK |
| | | SPDSLA | | LYSSNN | | GQPPKL | | S | | SGSGSG | | WALT | | VEIK |
| | | VSLGER | | KNNLA | | LIY | | | | TDFTLT | | | | |
| | | ATINC | | | | | | | | ISSLQA | | | | |
| | | | | | | | | | | EDVAVY | | | | |
| | | | | | | | | | | YC | | | | |
| ADI-81605 | IGKV4-1 | DIQMTQ | 1927 | KSSQSV | 2254 | WYQQKP | 4023 | WASTRE | 2422 | GVPDRF | 2619 | QQYYST | 4518 | FGGGTK |
| | | SPDSLA | | LYSSNN | | GQPPQL | | S | | SGSGSG | | PLT | | VEIK |
| | | VSLGER | | KNYLA | | LIY | | | | TDFTLT | | | | |
| | | ATINC | | | | | | | | ISSLQA | | | | |
| | | | | | | | | | | EDVAVY | | | | |
| | | | | | | | | | | YC | | | | |
| ADI-81870 | IGKV3-20 | ETTLTQ | 3628 | RASLSV | 3905 | WYQQKP | 2258 | GASSRA | 2464 | GIPDRF | 2616 | QQYGSS | 4585 | FGGGTK |
| | | SPGTLS | | SSSYLA | | GQAPRL | | T | | SGSGSG | | PLT | | VEIK |
| | | LSPGER | | | | LIY | | | | TDFTLT | | | | |
| | | ATLSC | | | | | | | | ISRLEP | | | | |
| | | | | | | | | | | EDFAVY | | | | |
| | | | | | | | | | | YC | | | | |
| ADI-81902 | IGKV3D-15 | EIVLTQ | 2033 | RASQSV | 2227 | WYQQKP | 2258 | GASTRA | 2383 | GIPARF | 2569 | QQYHNW | 4586 | FGGGTN |
| | | SPATLS | | SSNLA | | GQAPRL | | T | | SGSGSG | | PPLT | | VDIK |
| | | VSPGER | | | | LIY | | | | TEFTLT | | | | |
| | | ATLSC | | | | | | | | ISSLQS | | | | |
| | | | | | | | | | | EDFAVY | | | | |
| | | | | | | | | | | YC | | | | |
| ADI-81617 | IGLV3-21 | QHVLTQ | 3705 | GGNNIG | 2208 | WYQQKP | 2304 | YDSDRP | 2498 | GIPERF | 2545 | QVWDSS | 4587 | FGGGTK |
| | | PPSVSV | | SKSVH | | GQAPVL | | S | | SGSNSG | | WV | | VTVL |
| | | APGKTA | | | | VIY | | | | NTATLT | | | | |
| | | RITC | | | | | | | | ISRVEA | | | | |
| | | | | | | | | | | GDEADY | | | | |
| | | | | | | | | | | YC | | | | |
| ADI-81570 | IGKV3D-15 | EITMTQ | 3706 | RASQSV | 2227 | WYQQKP | 2258 | GASTRA | 2383 | GIPARF | 2569 | QQYNNW | 2897 | FGQGTK |
| | | SPATLS | | SSNLA | | GQAPRL | | T | | SGSGSG | | PRT | | VEIK |
| | | VSPGER | | | | LIY | | | | TEFTLT | | | | |
| | | ATLSC | | | | | | | | ISSLQS | | | | |
| | | | | | | | | | | EDFAVY | | | | |
| | | | | | | | | | | YC | | | | |
| ADI-81664 | IGKV3D-15 | EMTLTQ | 1916 | RASQSV | 2227 | WYQQKP | 2258 | GASTRA | 2383 | GIPARF | 2569 | QQYNNW | 4588 | FGQGTK |
| | | SPATLS | | SSNLA | | GQAPRL | | T | | SGSGSG | | PWT | | VDIK |
| | | VSPGER | | | | LIY | | | | TEFTLT | | | | |
| | | ATLSC | | | | | | | | ISSLQS | | | | |
| | | | | | | | | | | EDFAVY | | | | |
| | | | | | | | | | | YC | | | | |
| ADI-81666 | IGKV3-11 | EMTLTQ | 3534 | RASQSV | 2168 | WYQQKP | 2258 | DASNRA | 2448 | GIPARF | 2607 | QQRSNW | 4589 | FGGGTK |
| | | SPATLS | | SSYLA | | GQAPRL | | T | | SGSGSG | | PGT | | VEIK |
| | | LSPGER | | | | LIY | | | | TDFTLT | | | | |
| | | ATLSC | | | | | | | | ISSLEP | | | | |
| | | | | | | | | | | EDFAVY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81915 | 2936 | GAAATTGTGATGACGCAGTCTCCAGACTCCCTGGCTGTGTCTC | 4949 | EIVMTQSPDSLAVSL | 5302 | 1 | 1 |
| | | TGGGCGAGAGGGCCACCATCAACTGCAAGTCCAGCCAGAGTGT | | GERATINCKSSQSVL | | | |
| | | TTTATACAGCTCCAACAATAAGAACAACTTAGCTTGGTACCAG | | YSSNNKNNLAWYQQK | | | |
| | | CAGAAACCAGGACAGCCTCCTAAGCTGCTCATTTACTGGGCAT | | PGQPPKLLIYWASTR | | | |
| | | CTACCCGGGAATCCGGGGTCCCTGACCGATTCAGTGGCAGCGG | | ESGVPDRFSGSGSGT | | | |
| | | GTCTGGGACAGATTTCACTCTCACCATCAGCAGCCTGCAGGCT | | DFTLTISSLQAEDVA | | | |
| | | GAAGATGTGGCAGTTTATTACTGTCAGCAATATTATAGTACTT | | VYYCQQYYSTWALTF | | | |
| | | GGGCGCTCACTTTCGGCGGAGGGACCAAGGTGGAAATCAAA | | GGGTKVEIK | | | |
| ADI-81605 | 2936 | GACATCCAGATGACCCAGTCTCCAGACTCCCTGGCTGTGTCTC | 4950 | DIQMTQSPDSLAVSL | 5303 | 1 | 1 |
| | | TGGGCGAGAGGGCCACCATCAACTGCAAGTCCAGCCAGAGTGT | | GERATINCKSSQSVL | | | |
| | | TTTATACAGCTCCAACAATAAGAACTACTTAGCTTGGTACCAG | | YSSNNKNYLAWYQQK | | | |
| | | CAGAAACCAGGACAGCCTCCTCAGCTACTCATTTACTGGGCAT | | PGQPPQLLIYWASTR | | | |
| | | CTACCCGGGAATCCGGGGTCCCTGACCGATTCAGTGGCAGCGG | | ESGVPDRFSGSGSGT | | | |
| | | GTCTGGGACAGATTTCACTCTCACCATCAGCAGCCTGCAGGCT | | DFTLTISSLQAEDVA | | | |
| | | GAAGATGTGGCAGTTTATTACTGTCAGCAATATTATAGTACTC | | VYYCQQYYSTPLTFG | | | |
| | | CTCTCACTTTCGGCGGAGGGACCAAGGTGGAAATCAAA | | GGTKVEIK | | | |
| ADI-81870 | 2936 | GAAACGACACTCACGCAGTCTCCAGGCACCCTGTCTTTGTCTC | 4951 | ETTLTQSPGTLSLSP | 5304 | 1 | 1 |
| | | CAGGGGAAAGAGCCACCCTCTCCTGCAGGGCCAGTCTGAGTGT | | GERATLSCRASLSVS | | | |
| | | TAGCAGCAGCTACTTAGCCTGGTACCAGCAGAAACCTGGCCAG | | SSYLAWYQQKPGQAP | | | |
| | | GCTCCCAGGCTCCTCATCTATGGTGCATCCAGCAGGGCCACTG | | RLLIYGASSRATGIP | | | |
| | | GCATCCCAGACAGGTTCAGTGGCAGTGGGTCTGGGACAGACTT | | DRFSGSGSGTDFTLT | | | |
| | | CACTCTCACCATCAGCAGACTGGAGCCTGAAGATTTTGCAGTG | | ISRLEPEDFAVYYCQ | | | |
| | | TATTACTGTCAGCAGTATGGTAGCTCACCGCTCACTTTCGGCG | | QYGSSPLTFGGGTKV | | | |
| | | GAGGGACCAAGGTGGAGATCAAA | | EIK | | | |
| ADI-81902 | 4605 | GAAATTGTATTGACACAGTCTCCAGCCACCCTGTCTGTGTCTC | 4952 | EIVLTQSPATLSVSP | 5305 | 0 | 0 |
| | | CAGGGGAAAGAGCCACCCTCTCCTGCAGGGCCAGTCAGAGTGT | | GERATLSCRASQSVS | | | |
| | | TAGCAGCAATTTAGCCTGGTACCAGCAGAAACCTGGCCAGGCT | | SNLAWYQQKPGQAPR | | | |
| | | CCCAGGCTCCTCATCTATGGTGCATCCACCAGGGCCACTGGTA | | LLIYGASTRATGIPA | | | |
| | | TCCCAGCCAGGTTCAGTGGCAGTGGGTCTGGGACAGAGTTCAC | | RFSGSGSGTEFTLTI | | | |
| | | TCTCACCATCAGCAGCCTGCAGTCTGAAGATTTTGCAGTTTAT | | SSLQSEDFAVYYCQQ | | | |
| | | TACTGTCAGCAGTATCATAATTGGCCTCCGCTCACTTTCGGCG | | YHNWPPLTFGGGTNV | | | |
| | | GAGGGACCAACGTGGATATCAAA | | DIK | | | |
| ADI-81617 | 2947 | CAGCATGTGCTGACTCAGCCACCCTCAGTGTCAGTGGCCCCAG | 4953 | QHVLTQPPSVSVAPG | 5306 | 0 | 0 |
| | | GAAAGACGGCCAGGATTACCTGTGGGGGAAACAACATTGGAAG | | KTARITCGGNNIGSK | | | |
| | | TAAAAGTGTGCACTGGTACCAGCAGAAGCCAGGCCAGGCCCCT | | SVHWYQQKPGQAPVL | | | |
| | | GTGCTGGTCATCTATTATGATAGCGACCGGCCCTCAGGGATCC | | VIYYDSDRPSGIPER | | | |
| | | CTGAGCGATTCTCTGGCTCCAACTCTGGGAACACGGCCACCCT | | FSGSNSGNTATLTIS | | | |
| | | GACCATCAGCAGGGTCGAAGCCGGGGATGAGGCCGACTATTAC | | RVEAGDEADYYCQVW | | | |
| | | TGTCAGGTGTGGGATAGTAGTTGGGTGTTCGGCGGAGGGACCA | | DSSWVFGGGTKVTVL | | | |
| | | AGGTCACCGTCCTA | | | | | |
| ADI-81570 | 2937 | GAAATCACAATGACGCAGTCTCCAGCCACCCTGTCTGTGTCTC | 4954 | EITMTQSPATLSVSP | 5307 | 0 | 0 |
| | | CAGGGGAAAGAGCCACCCTCTCCTGCAGGGCCAGTCAGAGTGT | | GERATLSCRASQSVS | | | |
| | | CAGCAGCAACTTAGCCTGGTACCAGCAGAAACCTGGCCAGGCT | | SNLAWYQQKPGQAPR | | | |
| | | CCCAGGCTCCTCATCTATGGTGCATCCACCAGGGCCACTGGTA | | LLIYGASTRATGIPA | | | |
| | | TCCCAGCCAGGTTCAGTGGCAGTGGGTCTGGGACAGAGTTCAC | | RFSGSGSGTEFTLTI | | | |
| | | TCTCACCATCAGCAGCCTGCAGTCTGAAGATTTTGCAGTTTAT | | SSLQSEDFAVYYCQQ | | | |
| | | TACTGTCAGCAGTATAATAACTGGCCTCGAACGTTCGGCCAAG | | YNNWPRTFGQGTKVE | | | |
| | | GGACCAAGGTGGAGATCAAA | | IK | | | |
| ADI-81664 | 2944 | GAAATGACACTCACGCAGTCTCCAGCCACCCTGTCTGTGTCTC | 4955 | EMTLTQSPATLSVSP | 5308 | 0 | 0 |
| | | CAGGGGAAAGAGCCACCCTCTCCTGCAGGGCCAGTCAGAGTGT | | GERATLSCRASQSVS | | | |
| | | TAGCAGCAACTTAGCCTGGTACCAGCAGAAACCTGGCCAGGCT | | SNLAWYQQKPGQAPR | | | |
| | | CCCAGGCTCCTCATCTATGGTGCATCCACCAGGGCCACTGGTA | | LLIYGASTRATGIPA | | | |
| | | TCCCAGCCAGGTTCAGTGGCAGTGGGTCTGGGACAGAGTTCAC | | RFSGSGSGTEFTLTI | | | |
| | | TCTCACCATCAGCAGCCTGCAGTCTGAAGATTTTGCAGTTTAT | | SSLQSEDFAVYYCQQ | | | |
| | | TACTGTCAGCAGTATAATAACTGGCCTTGGACGTTCGGCCAAG | | YNNWPWTFGQGTKVD | | | |
| | | GGACCAAGGTGGATATCAAA | | IK | | | |
| ADI-81666 | 2936 | GAAATGACACTGACGCAGTCTCCAGCCACCCTGTCTTTGTCTC | 4956 | EMTLTQSPATLSLSP | 5309 | 0 | 0 |
| | | CAGGGGAAAGAGCCACCCTCTCCTGCAGGGCCAGTCAGAGTGT | | GERATLSCRASQSVS | | | |
| | | TAGCAGCTACTTAGCCTGGTACCAACAGAAACCTGGCCAGGCT | | SYLAWYQQKPGQAPR | | | |
| | | CCCAGGCTCCTCATCTATGATGCATCCAACAGGGCCACTGGCA | | LLIYDASNRATGIPA | | | |
| | | TCCCAGCCAGGTTCAGTGGCAGTGGGTCTGGGACAGACTTCAC | | RFSGSGSGTDFTLTI | | | |
| | | TCTCACCATCAGCAGCCTAGAGCCTGAAGATTTTGCAGTTTAT | | SSLEPEDFAVYYCQQ | | | |
| | | TACTGTCAGCAGCGTAGCAACTGGCCGGGGACTTTCGGGGAG | | RSNWPGTFGGGTKVE | | | |
| | | GGACCAAGGTGGAAATCAAA | | IK | | | |

| | | | SEQ | | SEQ | | SEQ | | SEQ | | SEQ | | SEQ | |
|---|---|---|---|---|---|---|---|---|---|---|---|---|---|---|
| | VL | VL | ID | VL | ID | VL | ID | VL | ID | VL | ID | VL | ID | VL |
| Clone ID | Germline | FR1 | NO: | CDR1 | NO: | FR2 | NO: | CDR2 | NO: | FR3 | NO: | CDR3 | NO: | FR4 |
| ADI-81809 | IGKV1D-39 | EIVMTQ | 1891 | RASQSI | 2050 | WYQQKP | 2257 | AASSLQ | 2391 | GVPSRF | 2534 | QQSYSN | 4590 | FGQGTR |
| | | SPSSLS | | SSYLN | | GKAPKL | | S | | SGSGSG | | PPVT | | LEIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |
| ADI-81539 | IGLV3-21 | QYVLTQ | 3707 | GGNNIG | 2208 | WYQQKP | 2283 | DDSDRP | 2399 | GIPERF | 2545 | QLWDGS | 4591 | FGGGTK |
| | | PPSVSV | | SKSVH | | GQAPVL | | S | | SGSNSG | | SDHPEV | | LTVL |
| | | APGQTA | | | | VVY | | | | NTATLT | | V | | |
| | | RITC | | | | | | | | ISRVEA | | | | |
| | | | | | | | | | | GDEADY | | | | |
| | | | | | | | | | | YC | | | | |
| ADI-81648 | IGKV1-12 | EITLTQ | 3708 | RASQGI | 2244 | WYQQKP | 2257 | AASSLQ | 2391 | GVPSRF | 2534 | QQANSF | 2710 | FGGGTK |
| | | SPSSVS | | SSWLA | | GKAPKL | | S | | SGSGSG | | PLT | | VDIK |
| | | ASVGDR | | | | LIY | | | | TDFTLT | | | | |
| | | VTITC | | | | | | | | ISSLQP | | | | |
| | | | | | | | | | | EDFATY | | | | |
| | | | | | | | | | | YC | | | | |

| | | | | | | VL | VL Amino |
|---|---|---|---|---|---|---|---|
| | SEQ | | SEQ | | SEQ | Nucleotide | Acid |
| | ID | | ID | | ID | Sub- | Sub- |
| Clone ID | NO: | VL DNA | NO: | VL Protein | NO: | stitutions | stitutions |
| ADI-81809 | 2940 | GAAATTGTGATGACGCAGTCTCCATCCTCCCTGTCTGCATCTG | 4957 | EIVMTQSPSSLSASV | 5310 | 0 | 0 |
| | | TAGGAGACAGAGTCACCATCACTTGCCGGGCAAGTCAGAGCAT | | GDRVTITCRASQSIS | | | |
| | | TAGCAGCTATTTAAATTGGTATCAGCAGAAACCAGGGAAAGCC | | SYLNWYQQKPGKAPK | | | |
| | | CCTAAGCTCCTGATCTATGCTGCATCCAGTTTGCAAAGTGGGG | | LLIYAASSLQSGVPS | | | |
| | | TCCCATCAAGGTTCAGTGGCAGTGGATCTGGGACAGATTTCAC | | RFSGSGSGTDFTLTI | | | |
| | | TCTCACCATCAGCAGTCTGCAACCTGAAGATTTCGCAACTTAC | | SSLQPEDFATYYCQQ | | | |
| | | TACTGTCAACAGAGTTACAGTAACCCTCCAGTAACCTTCGGCC | | SYSNPPVTFGQGTRL | | | |
| | | AAGGGACACGACTGGAGATTAAA | | EIK | | | |
| ADI-81539 | 2939 | CAGTATGTGCTGACTCAGCCACCCTCGGTGTCAGTGGCCCCAG | 4958 | QYVLTQPPSVSVAPG | 5311 | 0 | 0 |
| | | GACAGACGGCCAGGATTACCTGTGGGGGAAACAACATTGGAAG | | QTARITCGGNNIGSK | | | |
| | | TAAAAGTGTGCACTGGTACCAGCAGAAGCCAGGCCAGGCCCCT | | SVHWYQQKPGQAPVL | | | |
| | | GTGCTGGTCGTCTATGATGATAGCGACCGGCCCTCAGGGATCC | | VVYDDSDRPSGIPER | | | |
| | | CTGAGCGATTCTCTGGCTCCAACTCTGGGAACACGGCCACCCT | | FSGSNSGNTATLTIS | | | |
| | | GACCATCAGCAGGGTCGAAGCCGGGGATGAGGCCGACTATTAC | | RVEAGDEADYYCQLW | | | |
| | | TGTCAGCTGTGGGATGGTAGTAGTGATCATCCGGAGGTGGTAT | | DGSSDHPEVVFGGGT | | | |
| | | TCGGCGGAGGGACCAAGCTCACCGTCCTA | | KLTVL | | | |
| ADI-81648 | 2938 | GAAATCACACTGACCCAGTCTCCATCTTCCGTGTCTGCATCTG | 4959 | EITLTQSPSSVSASV | 5312 | 0 | 0 |
| | | TAGGAGACAGAGTCACCATCACTTGTCGGGCGAGTCAGGGTAT | | GDRVTITCRASQGIS | | | |
| | | TAGCAGCTGGTTAGCCTGGTATCAGCAGAAACCAGGGAAAGCC | | SWLAWYQQKPGKAPK | | | |
| | | CCTAAGCTCCTGATCTATGCTGCATCCAGTTTGCAAAGTGGGG | | LLIYAASSLQSGVPS | | | |
| | | TCCCATCAAGGTTCAGCGGCAGTGGATCTGGGACAGATTTCAC | | RFSGSGSGTDFTLTI | | | |
| | | TCTCACCATCAGCAGCCTGCAGCCTGAAGATTTTGCAACTTAC | | SSLQPEDFATYYCQQ | | | |
| | | TATTGTCAACAGGCTAACAGTTTCCCTCTCACTTTCGGCGGAG | | ANSFPLTFGGGTKVD | | | |
| | | GGACCAAAGTGGATATCAAA | | IK | | | |

1. An isolated antibody, or antigen-binding fragment thereof, that binds to the spike protein of a coronavirus (CoV-S), wherein said antibody, or antigen-binding fragment thereof, comprises a heavy chain variable region (VH) and a light chain variable region (VL),
wherein the VH comprises an amino acid sequence having at least 90%, 91%, 92%, 93%, 94%, 95%, 96%, 97%, 98%, or 99% sequence identity to, comprises, or consists of an amino acid sequence selected from the group consisting of any one of the VH sequences in Tables 3 and 5, and
wherein the VL comprises an amino acid sequence having at least 90%, 91%, 92%, 93%, 94%, 95%, 96%, 97%, 98%, or 99% sequence identity to, comprises, or consists of an amino acid sequence selected from the group consisting of any one of the VL sequences in Tables 4 and 6.

2. The isolated antibody, or antigen-binding fragment thereof, of claim 1, wherein the VH comprises a VH CDR1 amino acid sequence selected from the group consisting of any VH CDR1 sequence in Tables 3 and 5, a VH CDR2 amino acid sequence selected from the group consisting of any VH CDR2 sequence in Tables 3 and 5, and a VH CDR3 amino acid sequence selected from the group consisting of any VH CDR3 sequence in Tables 3 and 5, and
wherein the VL comprises a VL CDR1 amino acid sequence selected from the group consisting of any VL CDR1 sequence in Tables 4 and 6, a VL CDR2 amino acid sequence selected from the group consisting of any VL CDR2 sequence in Tables 4 and 6, and a VL CDR3 amino acid sequence selected from the group consisting of any VL CDR3 sequence in Tables 4 and 6.

3. An isolated antibody, or antigen-binding fragment thereof, that binds to the spike protein of a coronavirus (CoV-S), wherein said antibody, or antigen-binding fragment thereof, comprises a heavy chain variable region (VH) and a light chain variable region (VL),
wherein the VH comprises a VH CDR1 amino acid sequence selected from the group consisting of any VH CDR1 sequence in Tables 3 and 5, a VH CDR2 amino acid sequence selected from the group consisting of any VH CDR2 sequence in Tables 3 and 5, and a VH CDR3 amino acid sequence selected from the group consisting of any VH CDR3 sequence in Tables 3 and 5, and
wherein the VL comprises a VL CDR1 amino acid sequence selected from the group consisting of any VL CDR1 sequence in Tables 4 and 6, a VL CDR2 amino acid sequence selected from the group consisting of any VL CDR2 sequence in Tables 4 and 6, and a VL CDR3 amino acid sequence selected from the group consisting of any VL CDR3 sequence in Tables 4 and 6.

4. The isolated antibody, or antigen-binding fragment thereof, of claim 3,
wherein the VH comprises an amino acid sequence having at least 90%, 91%, 92%, 93%, 94%, 95%, 96%, 97%, 98%, or 99% sequence identity to, comprises, or consists of an amino acid sequence selected from the group consisting of any one of the VH sequences in Tables 3 and 5, and
wherein the VL comprises an amino acid sequence having at least 90%, 91%, 92%, 93%, 94%, 95%, 96%, 97%, 98%, or 99% sequence identity to, comprises, or consists of an amino acid sequence selected from the group consisting of any one of the VL sequences in Tables 4 and 6.

5. The isolated antibody, or antigen-binding fragment thereof, of claim 1,
(a) wherein the CoV-S is the spike protein of SARS-COV (“SARS-COV-S”) and/or the spike protein of SARS-COV-2 (“SARS-COV-2-S”);
(b) wherein SARS-COV-S comprises a sequence having at least 95% identity to the amino acid sequence of SEQ ID NO:1, and wherein SARS-COV-2-S comprises a sequence having at least 95% identity to the amino acid sequence of SEQ ID NO:5; and/or
(c) wherein the SARS-COV-2-S is a B.1.1.7 variant, a B.1.351 variant, a B.1.1.28 variant, a B.1.429 variant, a P.1 variant, a B.1.617 variant, a B.1.617.2 variant, a C.37 variant, a 1.621 variant, a AY.1 variant, a 1.623 variant, a C.36 variant, a A.27 variant, a AV.1 variant, a B.1.1.482 variant, a B.1.1.523 variant, a B.1.427 variant, a AY.4 variant, a AY.11 variant, a D614G variant, a B.1.1.529/BA.1 variant, a BA1.1 variant, a BA.2 variant, a BA.2.75 variant, a BA.4 variant, a BA.5 variant, a BA.4.6 variant, a BQ.1 variant, a BQ.1.1 variant, a XBB variant, a XBB.1 variant, a XBB.1.5 variant, a BJ.1 variant, a BM.1.1.1 variant, a BA.2.3.20 variant, a BF.7 variant, a XBC variant, a BN.1 variant, or a CH.1.1 variant.

6. The isolated antibody, or antigen-binding fragment thereof, of claim 1, wherein the VH comprises the VH amino acid sequence of ADI-75865, VYD225, ADI-80707, ADI-75696, ADI-75864, ADI-75620, ADI-75738, ADI-75700, ADI-75859, ADI-75684, ADI-75754, ADI-75648, ADI-75632, ADI-75741, ADI-75725, ADI-75717, ADI-75706, ADI-75699, ADI-75747, or ADI-75773; and
wherein the VL comprises the VL amino acid sequence of ADI-75865, VYD225, ADI-80707, ADI-75696, ADI-75864, ADI-75620, ADI-75738, ADI-75700, ADI-75859, ADI-75684, ADI-75754, ADI-75648, ADI-75632, ADI-75741, ADI-75725, ADI-75717, ADI-75706, ADI-75699, ADI-75747, or ADI-75773.

7. The isolated antibody, or antigen-binding fragment thereof, of claim 1, comprising the VH CDR1, VH CDR2, VH CDR3, VL CDR1, VL CDR2, and VL CDR3 amino acid sequences of ADI-75865, VYD225, ADI-80707, ADI-75696, ADI-75864, ADI-75620, ADI-75738, ADI-75700, ADI-75859, ADI-75684, ADI-75754, ADI-75648, ADI-75632, ADI-75741, ADI-75725, ADI-75717, ADI-75706, ADI-75699, ADI-75747, or ADI-75773.

8. (canceled)

9. (canceled)

10. The isolated antibody, or antigen-binding fragment thereof, of claim 1,
(a) wherein the isolated antibody, or antigen-binding fragment thereof, cross-reacts with SARS-COV-S and SARS-COV-2-S;
(b) wherein the antibody, or antigen-binding fragment thereof, binds to the receptor binding domain (RBD) or the N-terminal domain (NTD) of SARS-COV-S and/or of SARS-COV-2-S;
(c) wherein the antibody, or antigen-binding fragment thereof, binds to the receptor binding domain (RBD) of CoV-S from the B.1.1.529/BA.1 variant, the BA.1.1 variant, the BA.2.75 variant, the BQ.1.1 variant, and/or the XBB variant;
(d) wherein the antibody, or antigen-binding fragment thereof, binds to the S1 subunit and/or the S2 subunit of SARS-COV-S and/or of SARS-COV-2-S;
(e) wherein the antibody, or antigen-binding fragment thereof, binds to the ACE2-binding motif of SARS-COV-S and/or of SARS-COV-2-S;
(f) wherein the antibody, or antigen-binding fragment thereof, competes for binding with ACE2;
(g) wherein the antibody, or antigen-binding fragment thereof, binds to the S protein of SARS-COV and/or of SARS-COV-2; and does not bind to any of the S proteins of HCoV-229E, HCoV-HKU1, HCoV-NL63, and HCoV-OC43;
(h) wherein the antibody, or antigen-binding fragment thereof, binds to the S protein of SARS-COV and/or of SARS-COV-2; and binds to the S protein of at least one of HCoV-229E, HCoV-HKU1, HCoV-NL63, and HCoV-OC43;
(i) wherein the antibody, or antigen-binding fragment thereof, binds to CoV-S with a K_{D} value of:
(i) about 100 nM or lower;
(ii) about 10 nM or lower;
(iii) about 1 nM or lower;
(iv) about 100 pM or lower;
(v) about 10 pM or lower;
(vi) about 1 pM or lower; or
(vii) about 0.1 pM or lower;
(j) wherein the antibody, or antigen-binding fragment thereof, binds to the receptor binding domain (RBD) of CoV-S from the B.1.1.529/BA.1 variant, the BA.1.1 variant, the BA.2.75 variant, the BQ.1.1 variant, the XBB variant, the B.1351 variant, or the B.1.617.2 variant with a K_{D} value of about 100 nM or lower, or about 10 nM or lower, or about 1 nM or lower;
(k) wherein the antibody, or antigen-binding fragment thereof, neutralizes SARS-COV and/or SARS-COV-2;
(l) wherein the antibody, or antigen-binding fragment thereof, neutralizes SARS-COV and/or SARS-COV-2 at: (i) an IC50 of about 100 nM or lower, of about 50 nM or lower, of about 20 nM or lower, of about 10 nM or lower, of about 5 nM or lower, of about 2 nM or lower, of about 1 nM or lower, of about 500 pM or lower, of about 200 pM or lower, of about 100 pM or lower, of about 50 pM or lower, of about 20 pM or lower, of about 10 pM or lower, of about 5 pM or lower, of about 2 pM or lower, or of about 1 pM or lower; and/or (ii) an IC50 of about 1 μg/mL or lower, of about 500 ng/mL or lower, of about 200 ng/mL or lower, of about 100 ng/ml or lower, of about 50 ng/ml or lower, of about 40 ng/ml or lower, of about 30 ng/ml or lower, of about 20 ng/ml or lower, of about 10 mg/mL or lower, of about 5 ng/mL or lower, of about 2 ng/ml or lower, or of about 1 ng/ml or lower, in vitro;
(m) wherein the antibody, or antigen-binding fragment thereof, neutralizes the B.1.1.529/BA.1 variant of SARS-COV-2 with an IC50 of about 100 ng/ml or lower, of about 50 ng/ml or lower, of about 40 ng/ml or lower, of about 30 ng/ml or lower, of about 20 ng/ml or lower, of about 10 mg/mL or lower, of about 5 ng/ml or lower, of about 2 ng/ml or lower, or of about 1 ng/ml or lower, in vitro;
(n) wherein the antibody, or antigen-binding fragment thereof, neutralizes the BA.2.75 variant, the BF.7 variant, the BQ.1.1 variant, the XBB.1 variant, and/or the XBB.1.5 variant of SARS-COV-2 with an IC50 of about 200 ng/ml or lower, of about 100 ng/ml or lower, of about 50 ng/ml or lower, of about 40 ng/ml or lower, of about 30 ng/ml or lower, of about 20 ng/ml or lower, of about 10 mg/mL or lower, of about 5 ng/ml or lower, of about 2 ng/mL or lower, or of about 1 ng/mL or lower, in vitro;
(o) wherein the antibody, or antigen-binding fragment thereof, is a human, humanized, primatized, chimeric, bispecific, or multispecific antibody, or antigen-binding fragment thereof;
(p) wherein the antibody, or antigen-binding fragment thereof, comprises a Fab, Fab2, or scFv; and/or
(q) wherein the antibody, or antigen-binding fragment thereof, comprises a constant region, an Fc region, or at least one domain thereof;
wherein the constant region or Fc region comprises a mutation which impairs at least one effector function, FcR binding, complement binding, glycosylation, complement-dependent cytotoxicity (“CDC”), or antibody-dependent cellular cytotoxicity (“ADCC”); or
wherein the constant or Fc region is a human constant or Fc region selected from a human IgG1, IgG2, IgG3 or IgG4 constant or Fc region.

11-32. (canceled)

33. An isolated antibody, or antigen-binding fragment thereof, which competes for binding with, or binds the same epitope as the isolated antibody, or antigen-binding fragment thereof, of claim 1.

34. (canceled)

35. An affinity matured variant of any one of the isolated antibody, or antigen-binding fragment thereof, of claim 1.

36. A chimeric antigen receptor (“CAR”) comprising at least one antibody, or antigen-binding fragment thereof, according to claim 1.

37. An antibody drug conjugate (“ADC”) comprising: (a) at least one antibody, or antigen-binding fragment thereof, according to claim 1; and (b) a drug.

38. (canceled)

39. (canceled)

40. A pharmaceutical composition comprising at least one antibody, or antigen-binding fragment thereof, of claim 1; and a pharmaceutically acceptable carrier or excipient.

41. A method of treating and/or preventing infection by SARS-CoV, SARS-COV-2, and/or another coronavirus optionally selected from the group consisting of MERS-COV, HCoV-HKU1, HCoV-OC43, HCOV-229E, and HCoV-NL63, or treating a condition, symptom, disease, or disorder associated with the infection in a subject in need thereof, comprising administering to the subject a therapeutically effective amount of the antibody, or antigen-binding fragment thereof, of claim 1.

42. (canceled)

43. (canceled)

44. A method of inducing an immune response against and/or inhibiting or blocking infection of susceptible cells by SARS-COV, SARS-COV-2, and/or another coronavirus optionally selected from the group consisting of MERS-COV, HCoV-HKU1, HCoV-OC43, HCoV-229E, and HCoV-NL63 in a subject in need thereof, comprising administering at least one antibody, or antigen-binding fragment thereof, according to claim 1.

45. (canceled)

46. (canceled)

47. A method of preventing the need for a subject infected with SARS-COV, SARS-COV-2, and/or another coronavirus optionally selected from the group consisting of MERS-COV, HCoV-HKU1, HCoV-OC43, HCOV-229E, and HCoV-NL63 to be placed on a ventilator, or reducing the time that a subject infected with SARS-COV or SARS-CoV-2 or another coronavirus optionally selected from the group consisting of MERS-COV, HCoV-HKU1, HCoV-OC43, HCOV-229E, and HCoV-NL63 is on a ventilator, comprising administering to the subject a prophylactically or therapeutically effective amount of an antibody, or antigen-binding fragment thereof, according to claim 1.

48. A method of preventing the onset of pneumonia in a subject infected SARS-COV, SARS-COV-2, and/or another coronavirus optionally selected from the group consisting of MERS-COV, HCoV-HKU1, HCoV-OC43, HCOV-229E, and HCoV-NL63, or treating pneumonia and/or the symptoms of pneumonia in a subject for a subject infected SARS-COV or SARS-COV-2 or another coronavirus optionally selected from the group consisting of MERS-COV, HCOV-HKU1, HCoV-OC43, HCOV-229E, and HCoV-NL63, comprising administering to the subject a prophylactically or therapeutically effective amount of an antibody, or antigen-binding fragment thereof, according to claim 1.

49-56. (canceled)

57. A method of producing the antibody, or antigen-binding portion thereof, of claim 1, the method comprising expressing the antibody, or antigen-binding portion thereof, in a recombinant cell, and isolating the antibody, or antigen-binding portion thereof, from the cell.

58-60. (canceled)

61. A composition comprising two or more isolated antibodies, or antigen-binding fragments thereof, selected from the group consisting of VYD225, VYD223 and VYD224.

62. (canceled)

63. An isolated nucleic acid molecule or an isolated mRNA molecule encoding the antibody, or antigen-binding fragment thereof, of claim 1.

64. (canceled)

65. (canceled)

66. A kit comprising the antibody, or antigen-binding fragment thereof, of claim 1, and instructions for use.

67. A vial comprising the antibody, or antigen-binding fragment thereof, of claim 1.

68. (canceled)

69. (canceled)
